SEQUENCE LISTING (2 sequences)

SEQ ID NO: 1 - protein, 1210 aa, Artificial [sequence omitted]

SEQ ID NO: 2 - protein, 1255 aa, Artificial [sequence omitted]

(19) United States
(12) Patent Application Publication
(10) Pub. No.: US 2023/0364070 A1
(43) Pub. Date: Nov. 16, 2023

(54) PYRROLO[3,2-C]PYRIDIN-4-ONE DERIVATIVES USEFUL IN THE TREATMENT OF CANCER

(71) Applicant: Scorpion Therapeutics, Inc., Boston, MA (US)

(72) Inventors: Benjamin C. Milgram, Weston, MA (US); Ryan D. White, Somerville, MA (US); David St. Jean, Jr., Natick, MA (US); Angel Guzman-Perez, Belmont, MA (US)

(21) Appl. No.: 18/027,316

(22) PCT Filed: Sep. 22, 2021
(86) PCT No.: PCT/US2021/051504

§ 371 Date: Mar. 20, 2023

Related U.S. Application Data
(60) Provisional application No. 63/082,324, filed on Sep. 23, 2020.
(60) Provisional application No. 63/092,970, filed on Oct. 16, 2020.

(51) Int. Cl. A61K 31/444; C07D 471/04; C07D 519/00; C07D 471/20; A61K 31/506; A61K 31/5377; A61K 39/395; A61K 31/517; A61K 31/4706

(52) U.S. Cl. CPC A61K 31/444; C07D 471/04; C07D 519/00; C07D 471/20; A61K 31/506; A61K 31/5377; A61K 39/3955; A61K 31/517; A61K 31/4706

(57) ABSTRACT

This disclosure provides chemical entities of Formula (I) (e.g., a compound or a pharmaceutically acceptable salt, and/or hydrate, and/or cocrystal, and/or drug combination of the compound) that inhibit epidermal growth factor receptor (EGFR, ERBB 1) and/or Human epidermal growth factor receptor 2 (HER2, ERBB2). These chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This disclosure also provides compositions containing the same as well as methods of using and making the same.

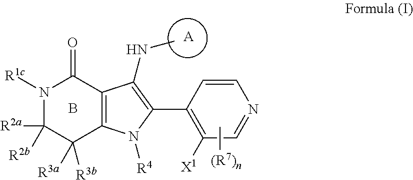

### CROSS REFERENCE TO RELATED APPLICATIONS

This application claims the benefit of U.S. Provisional Application Ser. No. 63/082,324, filed on Sep. 23, 2020; and U.S. Provisional Application Ser. No. 63/092,970, filed on Oct. 16, 2020; each of which is incorporated herein by reference in its entirety.

### TECHNICAL FIELD

This disclosure provides chemical entities (e.g., a compound or a pharmaceutically acceptable salt, and/or hydrate, and/or cocrystal, and/or drug combination of the compound) that inhibit epidermal growth factor receptor (EGFR, ERBB1) and/or Human epidermal growth factor receptor 2 (HER2, ERBB2). These chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This disclosure also provides compositions containing the same as well as methods of using and making the same.

### BACKGROUND

Epidermal growth factor receptor (EGFR, ERBB1) and Human epidermal growth factor receptor 2 (HER2, ERBB2) are members of a family of proteins which regulate cellular processes implicated in tumor growth, including proliferation and differentiation. Several investigators have demonstrated the role of EGFR and HER2 in development and cancer (Reviewed in Salomon, et al., Crit. Rev. Oncol. Hematol. (1995) 19:183-232, Klapper, et al., Adv. Cancer Res. (2000) 77, 25-79 and Hynes and Stern, Biochim. Biophys. Acta (1994) 1198:165-184). EGFR overexpression is present in at least 70% of human cancers, such as non-small cell lung carcinoma (NSCLC), breast cancer, glioma, and prostate cancer. HER2 overexpression occurs in approximately 30% of all breast cancer. It has also been implicated in other human cancers including colon, ovary, bladder, stomach, esophagus, lung, uterus and prostate. HER2 overexpression has also been correlated with poor prognosis in human cancer, including metastasis, and early relapse.

EGFR and HER2 are, therefore, widely recognized as targets for the design and development of therapies that can specifically bind and inhibit tyrosine kinase activity and its signal transduction pathway in cancer cells, and thus can serve as diagnostic or therapeutic agents. For example, EGFR tyrosine kinase inhibitors (TKIs) are effective clinical therapies for EGFR mutant advanced non-small cell lung cancer (NSCLC) patients. However, the vast majority of patients develop disease progression following successful treatment with an EGFR TKI. Common mechanisms of resistance include acquired, secondary mutation T790M, C797S, and EGFR exon 20 insertion mutations. For example, NSCLC tumors can have EGFR exon 20 insertion mutations that are intrinsically resistant to current EGFR TKIs.

Overexpression of another protein, BUB1 (Budding uninhibited by benzimidazole, BUB1) kinase, is often associated with proliferating cells, including cancer cells, and tissues (Bolanos-Garcia V M and Blundell T L, Trends Biochem. Sci. 36, 141, 2010). This protein is an essential part of the complex network of proteins that form the mitotic checkpoint. The major function of an unsatisfied mitotic checkpoint is to keep the anaphase-promoting complex/cyclosome (APC/C) in an inactive state. As soon as the checkpoint gets satisfied the APC/C ubiquitin-ligase targets cyclin B and securin for proteolytic degradation leading to separation of the paired chromosomes and exit from mitosis.

Incomplete mitotic checkpoint function has been linked with aneuploidy and tumourigenesis (see Weaver B A and Cleveland D W, Cancer Res. 67, 10103, 2007; King R W, Biochim Biophys Acta 1786, 4, 2008). In contrast, complete inhibition of the mitotic checkpoint has been recognized to result in severe chromosome missegregation and induction of apoptosis in tumour cells (see Kops G J et al., Nature Rev. Cancer 5, 773, 2005; Schmidt M and Medema R H, Cell Cycle 5, 159, 2006; Schmidt M and Bastians H, Drug Res. Updates 10, 162, 2007). Thus, mitotic checkpoint inhibition through inhibition of BUB1 kinase represents an approach for the treatment of proliferative disorders, including solid tumors such as carcinomas, sarcomas, leukemias and lymphoid malignancies or other disorders, associated with uncontrolled cellular proliferation.

### SUMMARY

This disclosure provides chemical entities (e.g., a compound or a pharmaceutically acceptable salt, and/or hydrate, and/or cocrystal, and/or drug combination of the compound) that inhibit epidermal growth factor receptor (EGFR, ERBB1) and/or Human epidermal growth factor receptor 2 (HER2, ERBB2). These chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This disclosure also provides compositions containing the same as well as methods of using and making the same.

In one aspect, the disclosure provides compounds of Formula (I):

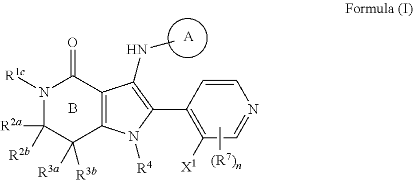

or a pharmaceutically acceptable salt thereof, wherein R^{1c}, R^{2a}, R^{2b}, R^{3a}, R^{3b}, Ring A, R⁴, X¹, R⁷, and n can be as defined anywhere herein.

In one aspect, the disclosure provides compounds of Formula (I):

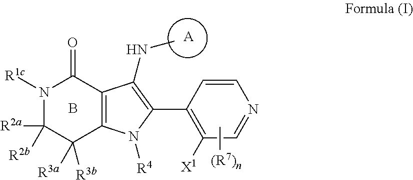

or a pharmaceutically acceptable salt thereof, wherein:
X¹ is selected from the group consisting of: (a) —O-L¹-R⁵; and (b);

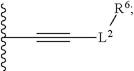

L¹ and L² are independently selected from the group consisting of: a bond and C₁₋₁₀ alkylene optionally substituted with from 1-6 R^{a};
R⁵ is selected from the group consisting of:
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
   C₆₋₁₀ aryl optionally substituted with from 1-4 R^{c};
   C₃₋₁₀ cycloalkyl or C₃₋₁₀ cycloalkenyl, each optionally substituted with from 1-4 substituents each independently selected from the group consisting of: oxo and R^{c};

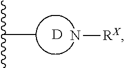

wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a};
—R^{g2}—R^{W} or —R^{g2}—R^{Y};
-L⁵-R^{g}; and
-L⁵-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
H; halo; —OH; —NR^{e}R^{f}; —R^{g}; -L⁶-R^{g}; —R^{g2}—R^{W} or —R^{g2}—R^{Y}; -L⁶-R^{g2}—R^{W} or -L⁶-R^{g2}—R^{Y}; and —C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a};
R^{Y} is selected from the group consisting of: R^{g} and -(L^{g})_{g}-R^{g};
each of R^{1c}, R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; NR^{e}R^{f}; R^{g}; and -(L^{g})_{g}-R^{g}; provided that R^{1c} is other than halo, —CN, or —C(O)OH; or
or two of variables R^{1c}, R^{2a}, R^{2b}, R^{3a}, and R^{3b}, together with the Ring B ring atoms to which each is attached, form a fused saturated or unsaturated ring of 3-12 ring atoms;
   wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
   wherein the fused saturated or unsaturated ring of 3-12 ring atoms is optionally substituted with from 1-4 substituents independently selected from the group consisting of oxo and R^{c};
Ring A is R^{g};
R⁴ is selected from the group consisting of: H and R^{d};
each R⁷ is an independently selected R^{c}; n is 0, 1, 2, or 3;
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; — S(O)₁₋₂(C₁₋₄ alkyl); —OH; and C₁₋₄ alkoxy;
each occurrence of R^{g} is independently selected from the group consisting of:
   C₃₋₁₀ cycloalkyl or C₃₋₁₀ cycloalkenyl, each of which is optionally substituted with from 1-4 substituents independently selected from the group consisting of oxo and R^{c};
   heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
   C₆₋₁₀ aryl optionally substituted with from 1-4 R^{c};
each occurrence of L⁹ is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and
each occurrence of R′ and R″ is independently selected from the group consisting of: H; —OH; and C₁₋₄ alkyl.

In some embodiments, it is provided that when R^{2a}, R^{2b}, R^{3a}, and R^{3b} are each H; R^{1c} is H or methyl; Ring A is phenyl optionally substituted with from 1-2 F; X¹ is —O-L¹-R⁵; and -L¹ is CH₂, then:
R⁵ is other than unsubstituted phenyl or unsubstituted cyclopropyl; and
further provided that the compound is other than: 3-((3-fluoro-2-methoxyphenyl)amino)-2-(3-((1-phenylpropan-2-yl)oxy)pyridin-4-yl)-1,5,6,7-tetrahydro-4H-pyrrolo[3,2-c]pyridin-4-one.

In one aspect, the disclosure features compounds of Formula (I):

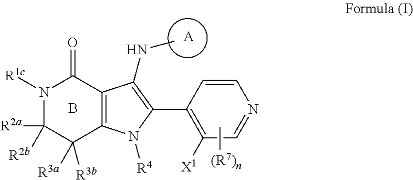

or a pharmaceutically acceptable salt thereof, wherein:
X¹ is selected from the group consisting of: (a) —O-L¹-R⁵; and (b)

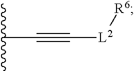

L¹ and L² are independently selected from the group consisting of: a bond and C₁₋₁₀ alkylene optionally substituted with from 1-6 R^{a};
R⁵ is selected from the group consisting of:
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
   C₆₋₁₀ aryl optionally substituted with from 1-4 R^{c},
   C₃₋₁₀ cycloalkyl or C₃₋₁₀ cycloalkenyl, each optionally substituted with from 1-4 substituents each independently selected from the group consisting of: oxo and R^{c};

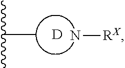

wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a};
—R^{W}
—R^{g2}—R^{W} or —R^{g2}—R^{Y};
-L⁵-R^{g}; and
-L⁵-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
H;
halo;
—OH;
—NR^{e}R^{f};
—R^{g};
—R^{W}
-L⁶-R^{g};
—R^{g2}—R^{W} or —R^{g2}—R^{Y};
-L⁶-R^{g2}—R^{W} or -L⁶-R^{g2}—R^{Y}; and
—C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a};
R^{Y} is selected from the group consisting of: —R and -(L^{g})_{g}-R^{g};
each of R^{1c}, R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g}; provided that R^{1c} is other than halo, —CN, or —C(O)OH; or
two of variables R^{1c}, R^{2a}, R^{2b}, R^{3a}, and R^{3b}, together with the Ring B ring atoms to which each is attached, form a fused saturated or unsaturated ring of 3-12 ring atoms;
   wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
   wherein the fused saturated or unsaturated ring of 3-12 ring atoms is optionally substituted with from 1-4 substituents independently selected from the group consisting of oxo, R^{c}, and R^{W};
Ring A is R^{g};
R⁴ is selected from the group consisting of: H and R^{d},
each R⁷ is an independently selected R^{c}; n is 0, 1, 2, or 3;
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₃₋₅ cycloalkyl; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₃₋₅ cycloalkyl optionally substituted with from 1-3 C₁₋₃ alkyl group; heterocyclyl including from 3-6 ring atoms, wherein from 1-3 ring atoms are heteroatoms, each independently selected from the group consisting of N, N(H), N(R^{d}), O, and S(O)₀₋₂ optionally substituted with from 1-4 substituents independently selected from the group consisting of oxo and R^{c}; and C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; a C₁₋₄ alkoxy;
each occurrence of R^{g} is independently selected from the group consisting of:
   C₃₋₁₀ cycloalkyl or C₃₋₁₀ cycloalkenyl, each of which is optionally substituted with from 1-4 substituents independently selected from the group consisting of oxo and R^{c};
   heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
   C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and
each occurrence of R′ and R″ is independently selected from the group consisting of: H; —OH; and C₁₋₄ alkyl; In some embodiments, when R^{2a}, R^{2b}, R^{3a}, and R^{3b} are each H; R^{1c} is H or methyl; Ring A is phenyl optionally substituted with from 1-2 F; X¹ is —O-L¹-R⁵; and -L¹ is CH₂, then:
R⁵ is other than unsubstituted phenyl or unsubstituted cyclopropyl; and

further provided that the compound is other than: 3-((3-fluoro-2-methoxyphenyl)amino)-2-(3-((1-phenylpropan-2-yl)oxy)pyridin-4-yl)-1,5,6,7-tetrahydro-4H-pyrrolo[3,2-c]pyridin-4-one.

Also provided herein is a pharmaceutical composition comprising a compound of Formula (I), or a pharmaceutically acceptable salt thereof, and a pharmaceutically acceptable carrier.

Provided herein is a method for treating cancer in a subject in need thereof, the method comprising administering to the subject a therapeutically effective amount of a compound of Formula (I), or a pharmaceutically acceptable salt thereof, or a pharmaceutical composition as provided herein.

Also provided herein is a method for treating cancer in a subject in need thereof, the method comprising (a) determining that the cancer is associated with a dysregulation of an EGFR gene, an EGFR kinase, or expression or activity or level of any of the same; and (b) administering to the subject a therapeutically effective amount of a compound of Formula (I), or a pharmaceutically acceptable salt thereof, or a pharmaceutical composition as provided herein.

Provided herein is a method of treating an EGFR-associated disease or disorder in a subject, the method comprising administering to a subject identified or diagnosed as having an EGFR-associated disease or disorder a therapeutically effective amount of a compound of Formula (I), or a pharmaceutically acceptable salt thereof, or a pharmaceutical composition as provided herein.

This disclosure also provides a method of treating an EGFR-associated disease or disorder in a subject, the method comprising: determining that the cancer in the subject is an EGFR-associated disease or disorder; and administering to the subject a therapeutically effective amount of a compound of Formula (I), or a pharmaceutically acceptable salt thereof, or a pharmaceutical composition as provided herein. Further provided herein is a method of treating an EGFR-associated cancer in a subject, the method comprising administering to a subject identified or diagnosed as having an EGFR-associated cancer a therapeutically effective amount of a compound of Formula (I), or a pharmaceutically acceptable salt thereof, or a pharmaceutical composition as provided herein.

This disclosure also provides a method of treating an EGFR-associated cancer in a subject, the method comprising: determining that the cancer in the subject is an EGFR-associated cancer; and administering to the subject a therapeutically effective amount of a compound of Formula (I), or a pharmaceutically acceptable salt thereof, or a pharmaceutical composition as provided herein.

Provided herein is a method of treating a subject, the method comprising administering a therapeutically effective amount of a compound of Formula (I), or a pharmaceutically acceptable salt thereof, or a pharmaceutical composition as provided herein, to a subject having a clinical record that indicates that the subject has a dysregulation of an EGFR gene, an EGFR kinase, or expression or activity or level of any of the same.

Also provided herein is a method of treating a subject having a cancer, wherein the method comprises:
(a) administering one or more doses of a first EGFR inhibitor to the subject for a period of time;
(b) after (a), determining whether a cancer cell in a sample obtained from the subject has at least one EGFR inhibitor resistance mutation that confers increased resistance to a cancer cell or tumor to treatment with the first EGFR inhibitor of step (a); and
(c) administering a therapeutically effective amount of a compound of Formula (I), or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first EGFR inhibitor of step (a); or
(d) administering additional doses of the first EGFR inhibitor of step (a) to the subject if the subject has not been determined to have a cancer cell that has at least one EGFR inhibitor resistance mutation that confers increased resistance to a cancer cell or tumor to treatment with the first EGFR inhibitor of step (a).

Further provided herein is a method of treating a subject having a cancer, wherein the method comprises:
(a) determining whether a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a therapeutically effective amount of a compound of Formula (I), or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first EGFR inhibitor that was previously administered to the subject; or
(c) administering additional doses of the first EGFR inhibitor to the subject if the subject has not been determined to have a cancer cell that has at least one EGFR inhibitor resistance mutation that confers increased resistance to a cancer cell or tumor to treatment with the first EGFR inhibitor previously administered to the subject.

Also provided herein is a method of treating a subject having a cancer, wherein the method comprises:
(a) determining that a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a therapeutically effective amount of a compound of Formula (I), or a pharmaceutically acceptable salt thereof, as a monotherapy or in conjunction with another anticancer agent to the subject.

Further provided herein is a method of treating a subject having a cancer, wherein the method comprises:
(a) determining that a cancer cell in a sample obtained from a subject having a cancer and previously administered one or more doses of a first EGFR inhibitor does not have one or more EGFR inhibitor resistance mutations that confer increased resistance to a cancer cell or tumor to treatment with the first EGFR inhibitor that was previously administered to the subject; and
(b) administering additional doses of the first EGFR inhibitor to the subject.

This disclosure also provides a method for inhibiting EGFR in a mammalian cell, the method comprising contacting the mammalian cell with an effective amount of a compound of Formula (I), or a pharmaceutically acceptable salt thereof.

Also provided herein is a method for treating cancer in a subject in need thereof, the method comprising (a) determining that the cancer is associated with a dysregulation of a HER2 gene, a HER2 kinase, or expression or activity or level of any of the same; and (b) administering to the subject a therapeutically effective amount of a compound of Formula (I), or a pharmaceutically acceptable salt thereof, or a pharmaceutical composition as provided herein.

Further provided herein is a method of treating a HER2-associated cancer in a subject, the method comprising administering to a subject identified or diagnosed as having a HER2-associated cancer a therapeutically effective amount of a compound of Formula (I), or a pharmaceutically acceptable salt thereof, or a pharmaceutical composition as provided herein.

This disclosure also provides a method of treating a HER2-associated cancer in a subject, the method comprising: determining that the cancer in the subject is a HER2-associated cancer; and administering to the subject a therapeutically effective amount of a compound of Formula (I), or a pharmaceutically acceptable salt thereof, or a pharmaceutical composition as provided herein.

Provided herein is a method of treating a subject having a cancer, the method comprising administering a therapeutically effective amount of a compound of Formula (I), or a pharmaceutically acceptable salt thereof, or a pharmaceutical composition as provided herein, to a subject having a clinical record that indicates that the subject has a dysregulation of a HER2 gene, a HER2 kinase, or expression or activity or level of any of the same.

Also provided herein is a method of treating a subject having a cancer, wherein the method comprises:
(a) administering one or more doses of a first HER2 inhibitor to the subject for a period of time;
(b) after (a), determining whether a cancer cell in a sample obtained from the subject has at least one HER2 inhibitor resistance mutation that confers increased resistance to a cancer cell or tumor to treatment with the first HER2 inhibitor of step (a); and
(c) administering a therapeutically effective amount of a compound of Formula (I), or a pharmaceutically acceptable salt thereof, as a monotherapy or in conjunction with another anticancer agent to the subject if the subject has been determined to have a cancer cell that has at least one HER2 inhibitor resistance mutation that confers increased resistance to a cancer cell or tumor to treatment with the first HER2 inhibitor of step (a); or
(d) administering additional doses of the first HER2 inhibitor of step (a) to the subject if the subject has not been determined to have a cancer cell that has at least one HER2 inhibitor resistance mutation that confers increased resistance to a cancer cell or tumor to treatment with the first HER2 inhibitor of step (a).

Further provided herein is a method of treating a subject having a cancer, wherein the method comprises:
(a) determining whether a cancer cell in a sample obtained from a subject having a cancer and previously administered one or more doses of a first HER2 inhibitor has one or more HER2 inhibitor resistance mutations that confer increased resistance to a cancer cell or tumor to treatment with the first HER2 inhibitor that was previously administered to the subject; and
(b) administering a therapeutically effective amount of a compound of Formula (I), or a pharmaceutically acceptable salt thereof, as a monotherapy or in conjunction with another anticancer agent to the subject if the subject has been determined to have a cancer cell that has at least one HER2 inhibitor resistance mutation that confers increased resistance to a cancer cell or tumor to treatment with the first HER2 inhibitor that was previously administered to the subject; or
(c) administering additional doses of the first HER2 inhibitor to the subject if the subject has not been determined to have a cancer cell that has at least one HER2 inhibitor resistance mutation that confers increased resistance to a cancer cell or tumor to treatment with the first HER2 inhibitor previously administered to the subject.

Also provided herein is a method of treating a subject having a cancer, wherein the method comprises:
(a) determining that a cancer cell in a sample obtained from a subject having a cancer and previously administered one or more doses of a first HER2 inhibitor has one or more HER2 inhibitor resistance mutations that confer increased resistance to a cancer cell or tumor to treatment with the first HER2 inhibitor that was previously administered to the subject; and
(b) administering a therapeutically effective amount of a compound of Formula (I), or a pharmaceutically acceptable salt thereof, as a monotherapy or in conjunction with another anticancer agent to the subject.

Further provided herein is a method of treating a subject having a cancer, wherein the method comprises:
(a) determining that a cancer cell in a sample obtained from a subject having a cancer and previously administered one or more doses of a first HER2 inhibitor does not have one or more HER2 inhibitor resistance mutations that confer increased resistance to a cancer cell or tumor to treatment with the first HER2 inhibitor that was previously administered to the subject; and
(b) administering additional doses of the first HER2 inhibitor to the subject.

This disclosure also provides a method for inhibiting HER2 in a mammalian cell, the method comprising contacting the mammalian cell with an effective amount of a compound of Formula (I), or a pharmaceutically acceptable salt thereof.

Also provided herein is a method for treating cancer in a subject in need thereof, the method comprising (a) determining that the cancer is associated with a dysregulation of an EGFR gene, an EGFR kinase, or expression or activity or level of any of the same and that the cancer is associated with a dysregulation of a HER2 gene, a HER2 kinase, or expression or activity or level of any of the same; and (b) administering to the subject a therapeutically effective amount of a compound of Formula (I), or a pharmaceutically acceptable salt thereof, or a pharmaceutical composition as provided herein.

Further provided herein is a method of treating an EGFR-associated and HER2-associated cancer in a subject, the method comprising administering to a subject identified or diagnosed as having an EGFR-associated and a HER2-associated cancer a therapeutically effective amount of a compound of Formula (I), or a pharmaceutically acceptable salt thereof, or a pharmaceutical composition as provided herein.

This disclosure also provides a method of treating a an EGFR-associated and HER2-associated cancer in a subject, the method comprising: determining that the cancer in the subject is an EGFR-associated and a HER2-associated cancer; and administering to the subject a therapeutically effective amount of a compound of Formula (I), or a pharmaceutically acceptable salt thereof, or a pharmaceutical composition as provided herein.

Provided herein is a method of treating a subject, the method comprising administering a therapeutically effective amount of a compound of Formula (I), or a pharmaceutically acceptable salt thereof, or a pharmaceutical composition as provided herein, to a subject having a clinical record that indicates that the subject has a dysregulation of an EGFR gene, an EGFR kinase, or expression or activity or level of any of the same and a dysregulation of a HER2 gene, a HER2 kinase, or expression or activity or level of any of the same.

This disclosure also provides a method for inhibiting EGFR and HER2 in a mammalian cell, the method comprising contacting the mammalian cell with an effective amount of a compound of Formula (I), or a pharmaceutically acceptable salt thereof.

In addition to the above, provided herein is a method for inhibiting a BUB (budding uninhibited by benzimidazole, BUB1-3) kinase. In some embodiments, the methods provided herein include methods for inhibiting BUB11. For example, a method for inhibiting BUB1 in a mammalian cell, the method comprising contacting the mammalian cell with an effective amount of a compound of Formula (I), or a pharmaceutically acceptable salt thereof.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as

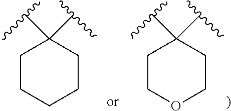 [SMILES: *C1(*)CCCCC1 ; *C1(*)CCOCC1]

or on different ring atoms (e.g., ring carbon and/or nitrogen atoms (e.g., vicinal ring carbon and/or nitrogen atoms))

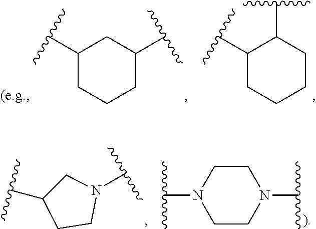

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents. Alkenyl groups can be trans or cis.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “cycloalkenyl” as used herein means partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. As partially unsaturated cyclic hydrocarbon groups, cycloalkenyl groups may have any degree of unsaturation provided that one or more double bonds is present in the ring, none of the rings in the ring system are aromatic, and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

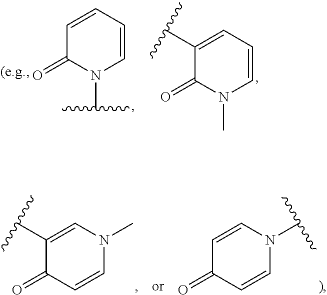

pyrimidone

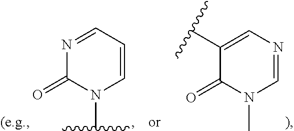

pyridazinone

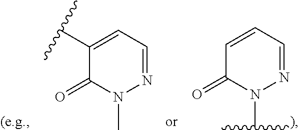

pyrazinone (e.g.,

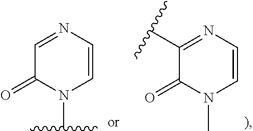

and imidazolone

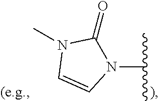 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like. The term “saturated” as used in this context means only single bonds present between constituent ring atoms and other available valences occupied by hydrogen and/or other substituents as defined herein.

The term “heterocycloalkenyl” as used herein means partially unsaturated cyclic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkenyl groups include, without limitation, tetrahydropyridyl, dihydropyrazinyl, dihydropyridyl, dihydropyrrolyl, dihydrofuranyl, dihydrothiophenyl. As partially unsaturated cyclic groups, heterocycloalkenyl groups may have any degree of unsaturation provided that one or more double bonds is present in the ring, none of the rings in the ring system are aromatic, and the heterocycloalkenyl group is not fully saturated overall. Heterocycloalkenyl may include multiple fused and/or bridged and/or spirocyclic rings. As used herein, examples of aromatic rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself, e.g., one or more double or tipple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

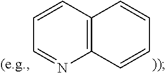

(ii) a single ring atom (spiro-fused ring systems)

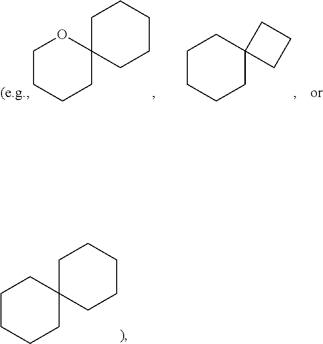

or (iii) a contiguous array of ring atoms (bridged ring systems having all bridge lengths >0)

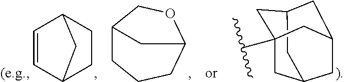

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

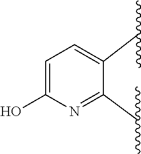 [SMILES: *c1ccc(O)nc1*]

encompasses the tautomeric form containing the moiety:

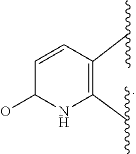 [SMILES: *C1=C(*)NC(O)C=C1]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

The details of one or more embodiments of the invention are set forth in the accompanying drawings and the description below. Other features and advantages of the invention will be apparent from the description and drawings, and from the claims.

### DETAILED DESCRIPTION

This disclosure provides chemical entities (e.g., a compound or a pharmaceutically acceptable salt, and/or hydrate, and/or cocrystal, and/or drug combination of the compound) that inhibit epidermal growth factor receptor (EGFR, ERBB1) and/or Human epidermal growth factor receptor 2 (HER2, ERBB2). These chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). In some embodiments, the chemical entities provided herein can inhibit an EGFR kinase and/or a HER2 kinase that has an exon 20 mutation (e.g., any of the exon 20 mutations described herein). Exon 20 mutations can confer intrinsic resistance to EGFR and/or HER2 inhibitors, and there are currently only limited targeted therapies that have been approved for subjects with these mutations. This disclosure also provides compositions containing the chemical entities provided herein as well as methods of using and making the same.

Formulae (I) Compounds

In one aspect, the disclosure features compounds of Formula (I):

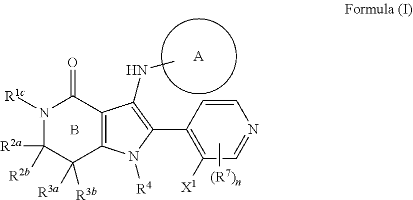

or a pharmaceutically acceptable salt thereof, wherein:
X¹ is selected from the group consisting of: (a) —O-L¹-R⁵; and (b)

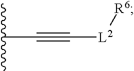

L¹ and L² are independently selected from the group consisting of: a bond and C₁₋₁₀ alkylene optionally substituted with from 1-6 R^{a};
R⁵ is selected from the group consisting of:
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
   C₆₋₁₀ aryl optionally substituted with from 1-4 R^{c};
   C₃₋₁₀ cycloalkyl or C₃₋₁₀ cycloalkenyl, each optionally substituted with from 1-4 substituents each independently selected from the group consisting of: oxo and R^{c};

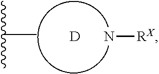

wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a};
—R^{g2}—R^{W} or —R^{g2}—R^{Y};
-L⁵-R^{g}; and
-L⁵-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
H;
halo;
—OH;
—NR^{e}R^{f};
—R^{g};
-L⁶-R^{g};
—R^{g2}—R^{W} or —R^{g2}—R^{Y};
-L⁶-R^{g2}—R^{W} or -L⁶-R^{g2}—R^{Y}; and
C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a};
R^{Y} is selected from the group consisting of: —R and -(L^{g})_{g}-R^{g};
each of R^{c}, R^{2a}, R^{2b}, R^{3a}, and R^{3A} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g}; provided that R^{1c} is other than halo, —CN, or —C(O)OH; or
or two of variables R^{1c}, R^{2a}, R^{2b}, R^{3a}, and R^{3b}, together with the Ring B ring atoms to which each is attached, form a fused saturated or unsaturated ring of 3-12 ring atoms;
   wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
   wherein the fused saturated or unsaturated ring of 3-12 ring atoms is optionally substituted with from 1-4 substituents independently selected from the group consisting of oxo, R^{c}, and R^{W};
Ring A is R^{g};
R⁴ is selected from the group consisting of: H and R^{d},
each R⁷ is an independently selected R^{c}; n is 0, 1, 2, or 3;
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
   C₃₋₁₀ cycloalkyl or C₃₋₁₀ cycloalkenyl, each of which is optionally substituted with from 1-4 substituents independently selected from the group consisting of oxo and R^{c};
   heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
   C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and each occurrence of R′ and R″ is independently selected from the group consisting of: H; —OH; and C₁₋₄ alkyl.

In one aspect, the disclosure features compounds of Formula (I):

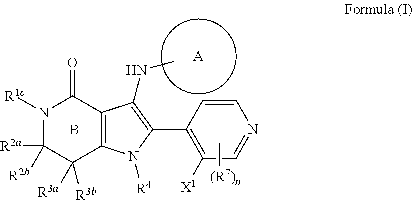

or a pharmaceutically acceptable salt thereof, wherein:
X¹ is selected from the group consisting of: (a) —O-L¹-R⁵; and (b);

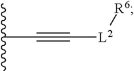

L¹ and L² are independently selected from the group consisting of: a bond and C₁₋₁₀ alkylene optionally substituted with from 1-6 R^{a};
R⁵ is selected from the group consisting of:
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
   C₆₋₁₀ aryl optionally substituted with from 1-4 R^{c};
   C₃₋₁₀ cycloalkyl or C₃₋₁₀ cycloalkenyl, each optionally substituted with from 1-4 substituents independently selected from the group consisting of: oxo and R^{c};

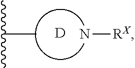

wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a};
—R^{g2}—R^{W} or —R^{g2}—R^{Y};
-L⁵-R^{g}; and
-L-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
H;
halo;
—OH;
—NR^{e}R^{f};
—R^{g};
-L⁶-R^{g};
—R^{g2}—R^{W} or —R^{g2}—R^{Y};
-L⁶-R^{g2}—R^{W} or -L⁶-R^{g2}—R^{Y}; and
—C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a};
R^{Y} is selected from the group consisting of: R^{g} and -(L^{g})_{g}-R^{g};
each of R^{1c}, R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; NR^{e}R^{f}; —R; and -(L^{g})_{g}-R^{g}; provided that R^{1c} is other than halo, —CN, or —C(O)OH; or
or two of variables R^{1c}, R^{2a}, R^{2b}, R^{3a}, and R^{3b}, together with the Ring B ring atoms to which each is attached, form a fused saturated or unsaturated ring of 3-12 ring atoms;
   wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
   wherein the fused saturated or unsaturated ring of 3-12 ring atoms is optionally substituted with from 1-4 substituents independently selected from the group consisting of oxo and R^{c};
Ring A is R^{g};
R⁴ is selected from the group consisting of: H and R^{d}
each R⁷ is an independently selected R^{c}; n is 0, 1, 2, or 3;
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
   C₃₋₁₀ cycloalkyl or C₃₋₁₀ cycloalkenyl, each of which is optionally substituted with from 1-4 substituents independently selected from the group consisting of oxo and R^{c};
   heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
   C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and each occurrence of R′ and R″ is independently selected from the group consisting of: H; —OH; and C₁₋₄ alkyl;

In some embodiments, it is provided that when R^{2a}, R^{2b}, R^{3a}, and R^{3b} are each H; R^{1c} is H or methyl; Ring A is phenyl optionally substituted with from 1-2 F; X¹ is —O-L¹-R⁵; and -L¹ is CH₂, then:
R⁵ is other than unsubstituted phenyl or unsubstituted cyclopropyl; and
further provided that the compound is other than: 3-((3-fluoro-2-methoxyphenyl)amino)-2-(3-((1-phenylpropan-2-yl)oxy)pyridin-4-yl)-1,5,6,7-tetrahydro-4H-pyrrolo[3,2-c]pyridin-4-one.

In one aspect, the disclosure features compounds of Formula (I):

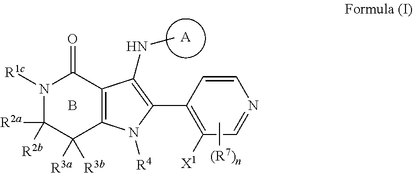

or a pharmaceutically acceptable salt thereof, wherein:

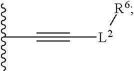

X¹ is selected from the group consisting of: (a) —O-L¹-R⁵; and (b);
L¹ and L² are independently selected from the group consisting of: a bond and C₁₋₁₀ alkylene optionally substituted with from 1-6 R^{a};
R⁵ is selected from the group consisting of:
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
   C₆₋₁₀ aryl optionally substituted with from 1-4 R^{c},
   C₃₋₁₀ cycloalkyl or C₃₋₁₀ cycloalkenyl, each optionally substituted with from 1-4 substituents each independently selected from the group consisting of: oxo and R^{c};

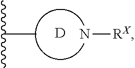

wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a};
—R^{W}
—R^{g2}—R^{W} or —R^{g2}—R^{Y};
-L⁵-R^{g}; and
-L⁵-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
   H;
   halo;
   —OH;
   —NR^{e}R^{f};
   —R^{g};
   —R^{W}
   -L⁶-R^{g};
   —R^{g2}—R^{W} or —R^{g2}—R^{Y};
   -L⁶-R^{g2}—R^{W} or -L⁶-R^{g2}—R^{Y}; and
—C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a};
R^{Y} is selected from the group consisting of: —R and -(L^{g})_{g}-R^{g};
each of R^{1c}, R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g}; provided that R^{1c} is other than halo, —CN, or —C(O)OH; or
two of variables R^{1c}, R^{2a}, R^{2b}, R^{3a}, and R^{3b}, together with the Ring B ring atoms to which each is attached, form a fused saturated or unsaturated ring of 3-12 ring atoms;
   wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
   wherein the fused saturated or unsaturated ring of 3-12 ring atoms is optionally substituted with from 1-4 substituents independently selected from the group consisting of oxo, R^{c}, and R^{W};
Ring A is R^{g};
R⁴ is selected from the group consisting of: H and R^{d},
each R⁷ is an independently selected R^{c}; n is 0, 1, 2, or 3;
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₃₋₈ cycloalkyl; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅; each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₃₋₅ cycloalkyl optionally substituted with from 1-3 C₁₋₃ alkyl group; heterocyclyl including from 3-6 ring atoms, wherein from 1-3 ring atoms are heteroatoms, each independently selected from the group consisting of N, N(H), N(R^{d}), O, and S(O)₀₋₂ optionally substituted with from 1-4 substituents independently selected from the group consisting of oxo and R^{c}; and C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; a C₁₋₄ alkoxy;
each occurrence of R^{g} is independently selected from the group consisting of:
   C₃₋₁₀ cycloalkyl or C₃₋₁₀ cycloalkenyl, each of which is optionally substituted with from 1-4 substituents independently selected from the group consisting of oxo and R^{c};
   heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
   C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and
each occurrence of R′ and R″ is independently selected from the group consisting of: H; —OH; and C₁₋₄ alkyl;

In some embodiments, when R^{2a}, R^{2b}, R^{3a}, and R^{3A} are each H; R^{1c} is H or methyl; Ring A is phenyl optionally substituted with from 1-2 F; X¹ is —O-L¹-R⁵; and -L¹ is CH₂, then:
R⁵ is other than unsubstituted phenyl or unsubstituted cyclopropyl; and

further provided that the compound is other than: 3-((3-fluoro-2-methoxyphenyl)amino)-2-(3-((1-phenylpropan-2-yl)oxy)pyridin-4-yl)-1,5,6,7-tetrahydro-4H-pyrrolo[3,2-c]pyridin-4-one.

In some embodiments, when R⁵ or R⁶ is heteroaryl, the heteroaryl is other than aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

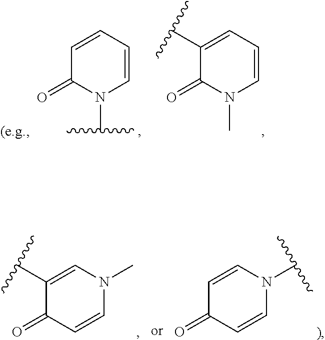

pyrimidone

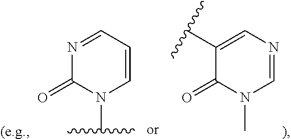

pyridazinone

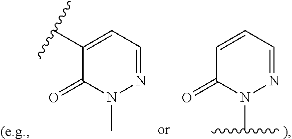

pyrazinone

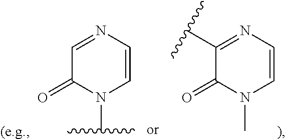

and imidazolone

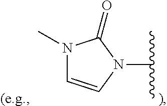

wherein each ring nitrogen adjacent to a carbonyl is tertiary (i.e., the oxo group (i.e., “═O”) herein is a constituent part of the heteroaryl ring).

In some embodiments, when R⁵ or R⁶ is heteroaryl, said heteroaryl is not substituted with —OH.

Variable X¹

Embodiments when X¹ is —O-L¹-R⁵

In some embodiments, X¹ is —O-L¹-R⁵.

In certain of these embodiments, R⁵ is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

In certain embodiments, R⁵ is a monocyclic heteroaryl including from 5-6 ring atoms, wherein from 1-4 ring atoms are heteroatoms, each independently selected from the group consisting of N, N(H), N(R^{d}), O, and S, and wherein the heteroaryl is optionally substituted with from 1-4 R^{cA}, wherein each R^{cA} is an independently selected R^{c}.

In certain of foregoing embodiments, R⁵ is monocyclic heteroaryl including 5 ring atoms, wherein from 1-4 ring atoms are heteroatoms, each independently selected from the group consisting of N, N(H), N(R^{d}), O, and S, and wherein the heteroaryl is optionally substituted with from 1-4 R^{cA}, wherein each R^{cA} is an independently selected R^{c}.

In certain embodiments, R⁵ is selected from the group consisting of furanyl, thiophenyl, oxadiazolyl, thiadiazolyl, triazolyl, tetrazolyl, imidazolyl, pyrazolyl, oxazolyl, and thiazolyl, each of which is optionally substituted with from 1-2 R^{cA}, and a ring nitrogen is optionally substituted with R^{d}, wherein each R^{cA} is an independently selected R^{c}.

As non-limiting examples of the foregoing embodiments, R⁵ can be selected from the group consisting of:

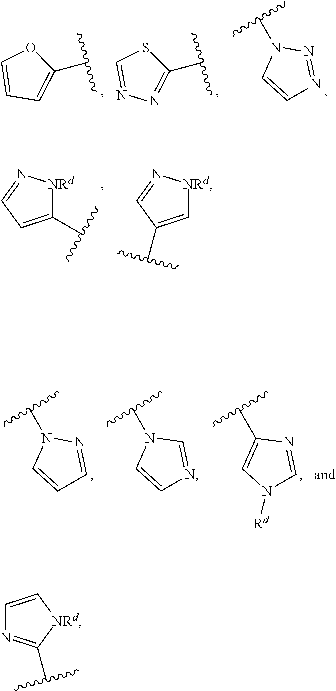

each optionally substituted with from 1-2 R^{cA}, wherein each R^{cA} is an independently selected R^{c}.

In certain embodiments, R⁵ is selected from the group consisting of furanyl, thiophenyl, oxadiazolyl, thiadiazolyl, tetrazolyl, imidazolyl, pyrazolyl, oxazolyl, and thiazolyl, each of which is optionally substituted with from 1-2 R^{cA}, and a ring nitrogen is optionally substituted with R^{d}, wherein each R^{cA} is an independently selected R^{c}.

As non-limiting examples of the foregoing embodiments, R⁵ can be selected from the group consisting of:

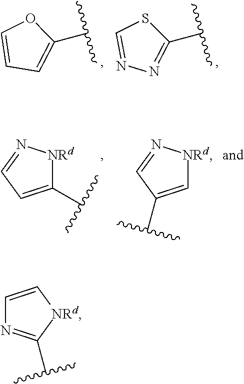

each optionally substituted with from 1-2 R^{cA}, wherein each R^{cA} is an independently selected R^{c}. For example, R⁵ can be

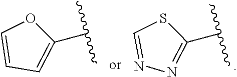 [SMILES: *c1ccco1 ; "or" ; *c1nncs1 ; "."]

As additional non-limiting examples, R⁵ can be

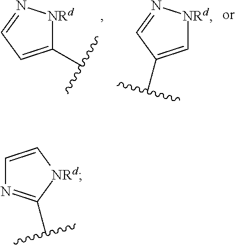

and optionally R^{d} can be C₁₋₃ alkyl.

In certain embodiments, R⁵ is monocyclic heteroaryl including 6 ring atoms, wherein from 1-4 ring atoms are ring nitrogen atoms, and wherein the heteroaryl is optionally substituted with from 1-4 R^{cA}, wherein each R^{cA} is an independently selected R^{c}.

In certain embodiments, R⁵ is selected from the group consisting of pyridyl, pyridonyl, pyrimidyl, pyrazinyl, and pyridazinyl, each optionally substituted with from 1-3 R^{cA}, wherein each R^{cA} is an independently selected R^{c}.

As non-limiting examples of the foregoing embodiments, R⁵ can be selected from the group consisting of:

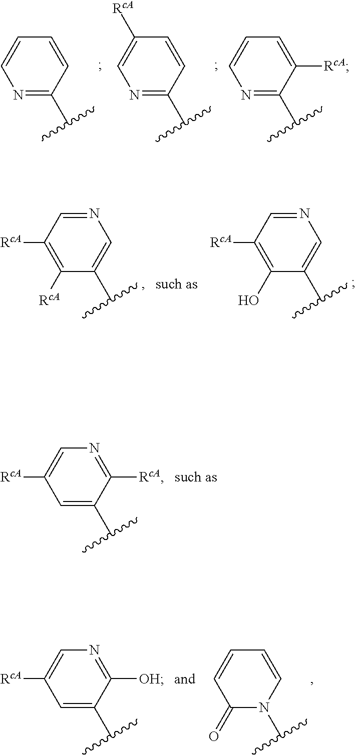

each of which is further optionally substituted with R^{cA}, wherein each R^{cA} is an independently selected R^{c}.

In certain of these embodiments, R⁵ is selected from the group consisting of pyridyl, pyrimidyl, pyrazinyl, and pyridazinyl, each optionally substituted with from 1-3 R^{cA}, wherein each R^{cA} is an independently selected R^{c}.

As non-limiting examples of the foregoing embodiments, R⁵ can be selected from the group consisting of:

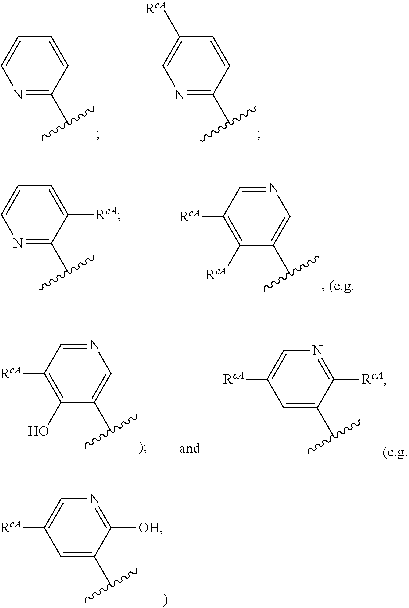

each of which is further optionally substituted with R^{cA}, wherein each R^{cA} is an independently selected R^{c}.

As further non-limiting examples, R⁵ can be selected from the group consisting of:

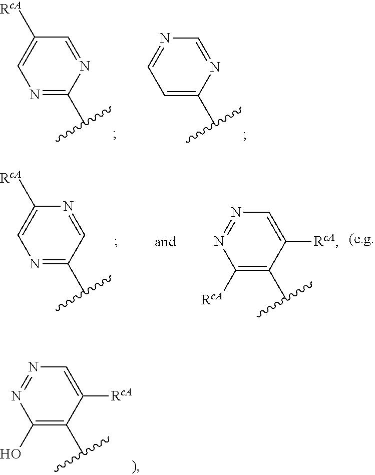

each of which is further optionally substituted with R^{cA}, wherein each R^{cA} is an independently selected R^{c}.

In certain embodiments, R⁵ is bicyclic heteroaryl including from 8-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

In certain of these embodiments, R⁵ is bicyclic heteroaryl including 8 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

As non-limiting examples of the foregoing embodiments, R⁵ can be selected from the group consisting of:

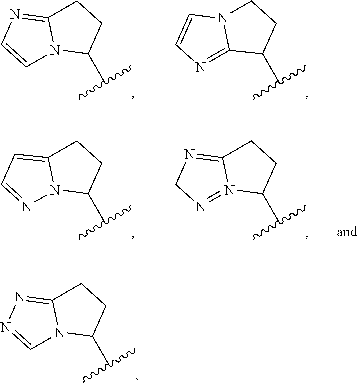

each of which is further optionally substituted with from 1-2 R^{cA}, wherein each R^{cA} is an independently selected R^{c}.

As further non-limiting examples, R⁵ can be selected from the group consisting of

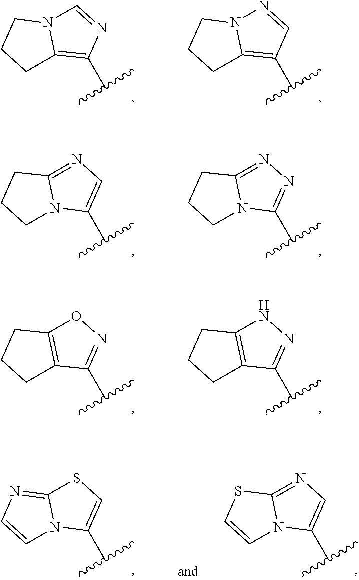

each of which is further optionally substituted with from 1-2 R^{cA}, wherein each R^{cA} is an independently selected R^{c}.

In certain embodiments, R⁵ is bicyclic heteroaryl including 9 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

In certain of these embodiments, R⁵ is imidazolopyridinyl, pyrazolopyridinyl, or benzotriazolyl, each of which is optionally substituted with from 1-2 R^{cA}, wherein each R^{cA} is an independently selected R^{c}.

As non-limiting examples of the foregoing embodiments, R⁵ can be

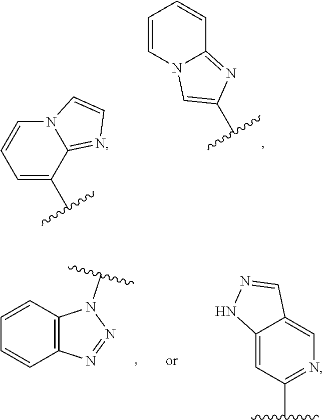 [SMILES: *c1cccn2ccnc12 ; *c1cn2ccccc2n1 ; *n1nnc2ccccc21 ; *c1cc2[nH]ncc2cn1]

each of which is optionally substituted with from 1-2 R^{cA}, wherein each R^{cA} is an independently selected R^{c}.

In certain embodiments, R⁵ is bicyclic 10-membered heteroaryl, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

In certain embodiments, each R^{cA} is independently selected from the group consisting of: halo; cyano; —OH; C₁₋₆ alkyl which is optionally substituted with from 1-6 independently selected R^{a}; C₁₋₄ alkoxy optionally substituted with C₁₋₄ alkoxy or C₁₋₄ haloalkoxy; C₁₋₄ haloalkoxy; and —C(═O)NR′R″.

In certain embodiments, one occurrence of R^{cA} is an independently selected halo, such as —F or —Cl.

In certain embodiments, one occurrence of R^{cA} is cyano.

In certain embodiments, one occurrence of R^{cA} is C₁₋₆ alkyl which is optionally substituted with from 1-6 independently selected R^{a}.

In certain embodiments, one occurrence of R^{cA} is C₁₋₆ alkyl, such as C₁₋₃ alkyl.

In certain embodiments, one occurrence of R^{cA} is C₁₋₆ alkyl substituted with —OH or —NR^{e}R^{f}. For example, one occurrence of R^{cA} can be C₁₋₃ alkyl substituted with —OH or NH₂.

In certain embodiments, one occurrence of R^{cA} is C₁₋₄ alkoxy optionally substituted with C₁₋₄ alkoxy or C₁₋₄ haloalkoxy. For example, one occurrence of R^{cA} can be C₁₋₄ alkoxy (e.g., methoxy or ethoxy).

In certain embodiments, one occurrence of R^{cA} is —C(═O)NR′R″ (e.g., C(═O)NH₂).

In certain embodiments, R⁵ is

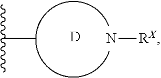

wherein Ring D is heterocyclylene or heterocycloalkenylene (e.g., heterocyclylene) including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene (e.g., heterocyclylene) is optionally substituted with from 1-4 substituents each independently selected from the group consisting of: oxo and —R^{c}.

In certain of these embodiments, R⁵ is

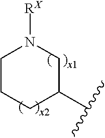

which is optionally substituted with from 1-2 R^{c}, wherein x1 and x2 are each independently 0, 1, or 2.

In certain of the foregoing embodiments, x1=0, and x2=0.

In certain embodiments, x1=0, and x2=1.

In certain embodiments, x1=0, and x2=2.

As non-limiting examples when R⁵ is

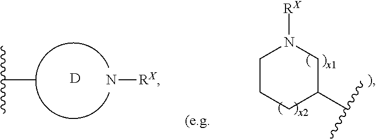

R⁵ can be selected from the group consisting of:

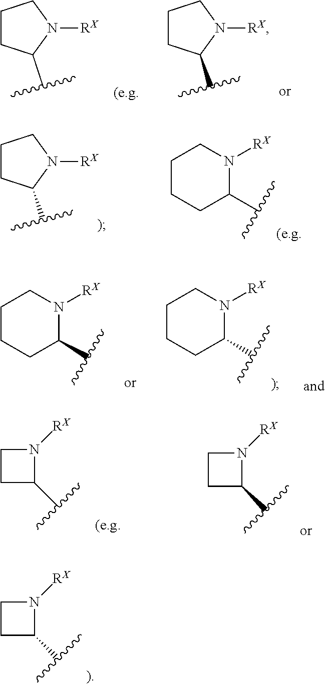

In certain embodiments, R^{X} is C(═O)(C₁₋₄ alkyl) or S(O)₂(C₁₋₄ alkyl).

In certain of these embodiments, R^{X} is C(═O)(C₁₋₄ alkyl) (e.g., C(═O)Me or C(═O)Et).

In certain embodiments, R^{X} is S(O)₂(C₁₋₄ alkyl) (e.g., S(O)₂Me).

In certain embodiments, R⁵ is —R^{g2}—R^{W}.

In certain of these embodiments, R⁵ is —R^{g2}—R^{W}; and the —R^{g2} present in —R^{g2}—R^{W} is heterocyclylene or heterocycloalkenylene including from 3-10 ring atoms, wherein from 1-3 ring atoms are heteroatoms, each independently selected from the group consisting of N, N(H), N(R^{d}), O, and S(O)₀₋₂, and wherein the heterocyclylene or heterocycloalkenylene is optionally substituted with from 1-4 substituents independently selected from the group consisting of oxo and R^{c}.

In certain of the foregoing embodiments, —R⁵ is

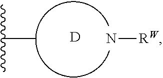

wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}.

In certain of these embodiments, —R⁵ is

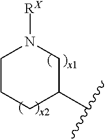

optionally substituted with from 1-2 R^{c}, wherein x1 and x2 are each independently 0, 1, or 2.

In certain of the foregoing embodiments, x1=0, and x2=0.

In certain embodiments, x1=0, and x2=1.

In certain embodiments, x1=0, and x2=2.

In certain embodiments, x1=0, and x2=1; or x1=0, and x2=2.

As non-limiting examples when R⁵ is

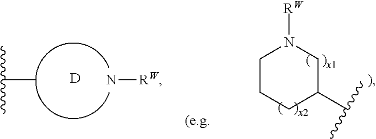

R⁵ can be selected from the group consisting of:

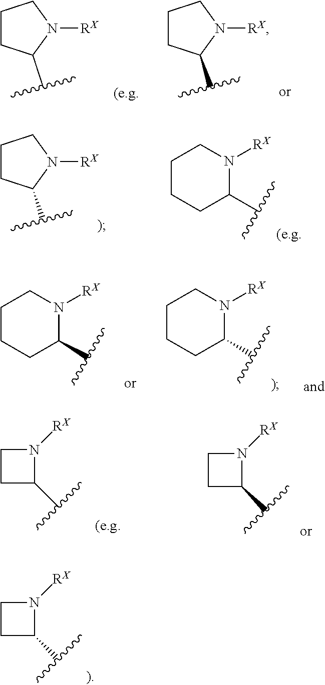

As non-limiting examples when R⁵ is

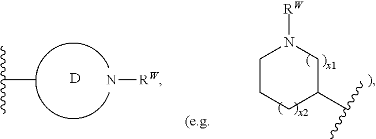

R⁵ can be selected from the group consisting of:

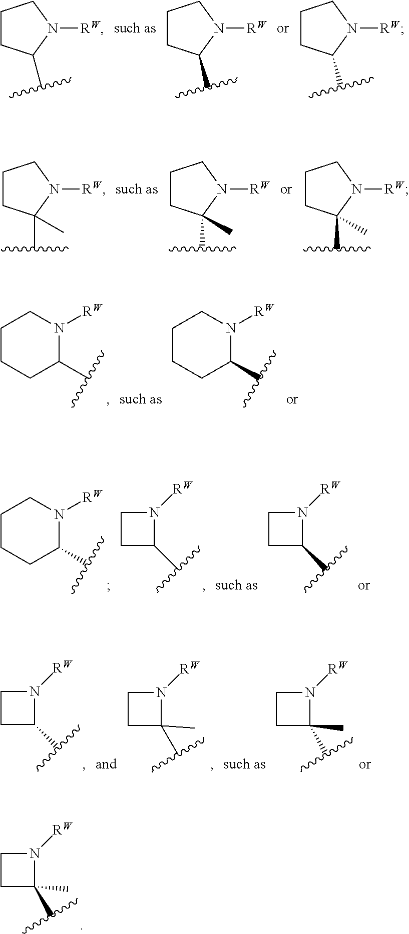

In some embodiments, R⁵ is R^{W}.

In certain embodiments, R^{W} is -L^{W}-W; and L^{W} is C(═O).

In certain embodiments, R^{W} is -L^{W}-W; and L^{W} is C(═O) NHC(═O)*, or NHS(O)₁₋₂* wherein the asterisk represents point of attachment to W.

In certain of these embodiments, W is C₂₋₆ alkenyl optionally substituted with from 1-3 R^{a} and further optionally substituted with R^{g}, wherein W is attached to L^{W} via an sp² hybridized carbon atom.

In certain of these embodiments, W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom.

In certain of the foregoing embodiments, W is C₂₋₄ alkenyl optionally substituted with from 1-3 R^{a} and further optionally substituted with R^{g}, wherein W is attached to L^{W} via an sp² hybridized carbon atom. As a non-limiting example of the foregoing embodiments, W can be CH═CH₂.

In certain of these foregoing embodiments, W is C₂₋₄ alkenyl or C₂₋₄ alkynyl optionally substituted with from 1-3 R^{a} and further optionally substituted with R^{g}, wherein W is attached to L^{W} via an sp² or sp hybridized carbon atom. As non-limiting examples of the foregoing embodiments, W can be CH═CH₂, CH═CHCH₂NMe₂, or

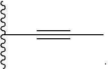 [SMILES: *C#CC]

In certain embodiments, -L^{W}-W is —C(═O)CH═CH₂.

As non-limiting examples, -L^{W}-W can be —C(═O)CH═CH₂, —C(═O)CH═CHCH₂NMe₂, or

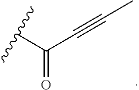 [SMILES: *C(=O)C#CC]

In certain embodiments, R⁵ is —R^{g2}—R^{Y}.

In certain of these embodiments, R⁵ is —R^{g2}—R^{Y}, wherein the —R^{g2} present in —R^{g2}—R^{Y} is heterocyclylene or heterocycloalkenylene including from 3-10 ring atoms, wherein from 1-3 ring atoms are heteroatoms, each independently selected from the group consisting of N, N(H), N(R^{d}), O, and S(O)₀₋₂, and wherein the heterocyclylene or heterocycloalkenylene is optionally substituted with from 1-3 substituents independently selected from the group consisting of oxo and R^{c}.

In certain embodiments (when R⁵ is —R^{g2}—R^{Y}), —R⁵ is

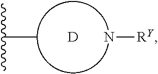

wherein Ring D is heterocyclylene including from 3-10 ring atoms, wherein from 0-2 ring atoms (in addition to the ring nitrogen atom bonded to R^{Y}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}.

In certain of the foregoing embodiments, —R⁵ is

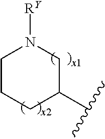

optionally substituted with from 1-2 R^{c}, wherein x1 and x2 are each independently 0, 1, or 2.

In certain of these embodiments, x1=0, and x2=0.

In certain embodiments, x1=0, and x2=1.

In certain embodiments, x1=0, and x2=2.

As non-limiting examples when R⁵ is

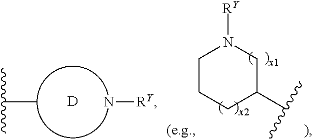

R⁵ can be selected from the group consisting of:

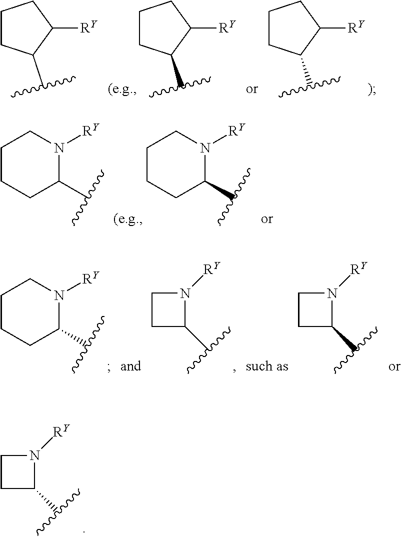

In certain embodiments, R⁵ is —R^{g2}—R^{Y}; and the —R^{g2} present in —R^{g2}—R is monocyclic heteroarylene including from 5-6 ring atoms, wherein from 1-4 ring atoms are heteroatoms, each independently selected from the group consisting of N, N(H), N(R^{d}), O, and S, and wherein the heteroarylene is optionally substituted with from 1-3 R^{c}.

In certain of these embodiments, R⁵ is —R^{g2}—R^{Y}; and the —R^{g2} present in —R^{g2}—R^{Y} is monocyclic heteroarylene including 5 ring atoms, wherein from 1-4 ring atoms are heteroatoms, each independently selected from the group consisting of N, N(H), N(R^{d}), O, and S, and wherein the heteroarylene is optionally substituted with from 1-2 R^{c}.

As non-limiting examples of the foregoing embodiments, R⁵ can be selected from the group consisting of:

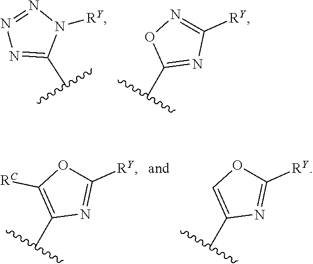

In certain embodiments (when R⁵ is —R^{g2}—R^{Y}), —R^{Y} is —R^{g}.

In certain of these embodiments, —R^{Y} is selected from the group consisting of:
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{e}.

In certain of the foregoing embodiments, —R^{Y} is C₆₋₁₀ aryl optionally substituted with from 1-4 R^{c}

As a non-limiting example of the foregoing embodiments, —R^{Y} can be phenyl optionally substituted with from 1-3 R^{c}.

In certain embodiments, —R^{Y} is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

In certain of these embodiments, —R is monocyclic heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

As non-limiting examples of the foregoing embodiments, —R^{Y} can be selected from the group consisting of pyridyl and pyrazolyl, each of which is optionally substituted with from 1-2 R^{c}.

In certain embodiments, R⁵ is C₃₋₁₀ cycloalkyl or C₃₋₁₀ cycloalkenyl, each optionally substituted with from 1-4 substituents each independently selected from the group consisting of: oxo and R^{c}.

In certain of these embodiments, R⁵ is C₃₋₁₀ cycloalkyl substituted with from 1-4 substituents each independently selected from the group consisting of: oxo and R^{c}.

In certain embodiments, R⁵ is C₃₋₆ cycloalkyl substituted with C₁₋₄ alkoxy or C₁₋₄ haloalkoxy; and R⁵ is further optionally substituted from 1-2 substituents each independently selected from the group consisting of: oxo and R^{c}.

In certain embodiments, R⁵ is cyclopropyl that is substituted with C₁₋₄ alkoxy or C₁₋₄ haloalkoxy. For example, R⁵ can be

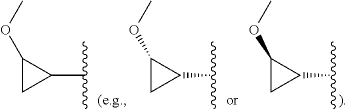

In certain embodiments, R⁵ is —S(O)₀₋₂(C₁₋₆ alkyl) which is optionally substituted with from 1-6 R^{a}.

In certain of these embodiments, R⁵ is —S(O)₂(C₁₋₆ alkyl) which is optionally substituted with from 1-6 R^{a}.

As a non-limiting example of the foregoing embodiments, R⁵ can be —S(O)₂(C₁₋₆ alkyl) (e.g., —S(O)₂(C₁₋₃ alkyl)).

In certain embodiments, R⁵ is selected from the group consisting of: -L⁵-R^{g}, -L⁵-R^{g2}—R^{Y}, and -L⁵-R^{g2}—R^{W}.

In certain of these embodiments, R⁵ is-L⁵-R^{g}. In certain of the foregoing embodiments, R⁵ is —O—R^{g}.

In certain embodiments, R⁵ is —O—R^{g}; and the R present in —O—R^{g} is C₃₋₁₀ cycloalkyl or C₃₋₁₀ cycloalkenyl, each optionally substituted with from 1-4 substituents each independently selected from the group consisting of oxo and R^{c}.

In certain embodiments, R⁵ is —O—(C₃₋₆ cycloalkyl), wherein the C₃₋₆ cycloalkyl is optionally substituted with from 1-3 R^{c}. For example, R⁵ can be

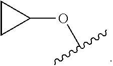 [SMILES: *OC1CC1]

In some embodiments, L¹ is C₁₋₁₀ alkylene optionally substituted with from 1-6 R^{a}.

In certain of these embodiments, L¹ is C₁₋₆ alkylene optionally substituted with from 1-6 R^{a}. In certain of the foregoing embodiments, L¹ is C₁₋₃ alkylene optionally substituted with from 1-6 R^{a}. In certain embodiments, L¹ is C₁₋₃ alkylene. For example, L¹ can be —CH₂. As another non-limiting example, L can be —CH₂CH₂—.

In certain of these embodiments, L¹ is C₁₋₄ alkylene optionally substituted with from 1-6 R^{a}. In certain of these foregoing embodiments, L¹ is C₁₋₄ alkylene. As non-limiting examples of the foregoing embodiments, L can be —CH₂— or —CH₂CH₂—. As another non-limiting examples of the foregoing embodiments, L¹ can be

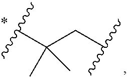 [SMILES: *CC(*)(C)C]

wherein the asterisk represents point of attachment to R^{W}.

In some embodiments, L¹ is a bond.

Embodiments when X¹ is

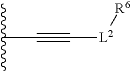

In some embodiments, X¹ is

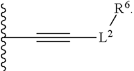

In certain of these embodiments, R⁶ is R^{g}.

In certain embodiments, R⁶ is heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

In certain embodiments, R⁶ is heterocyclyl including from 3-10 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

In certain of these embodiments, R⁶ is heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-2 substituents independently selected from the group consisting of oxo and R^{c}.

In certain embodiments, R⁶ is selected from the group consisting of pyrrolidinyl, piperidinyl, oxetanyl, tetrahydrofuranyl, and tetrahydropyranyl, each of which is optionally substituted with 1-2 substituents independently selected from the group consisting of oxo and R^{c}, wherein the ring nitrogen of the pyrrolidinyl or piperidinyl is optionally substituted with R^{d}, such as wherein R⁶ is

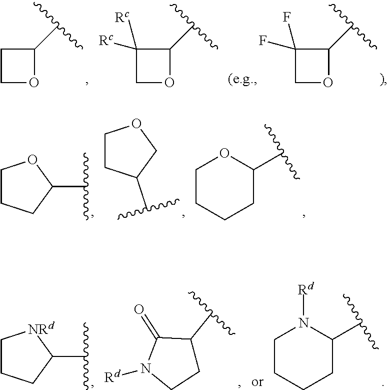

In certain of the foregoing embodiments, R⁶ is selected from the group consisting of pyrrolidinyl, piperidinyl, tetrahydrofuranyl, and tetrahydropyranyl, each of which is optionally substituted with 1-2 substituents independently selected from the group consisting of oxo and R^{c}, wherein the ring nitrogen of the pyrrolidinyl or piperidinyl is optionally substituted with R^{d}. As non-limiting examples of the foregoing embodiments, R⁶ can be

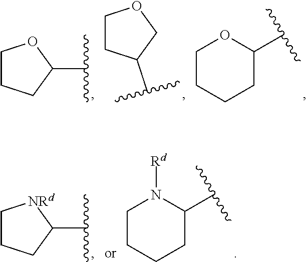

As another non-limiting example, R⁶ can be

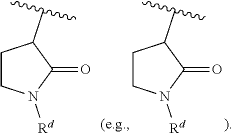

In certain embodiments (when R⁶ is R^{g}), R⁶ is C₃₋₈ cycloalkyl or C₃₋₈ cycloalkenyl, each optionally substituted with from 1-4 substituents independently selected from the group consisting of oxo and R^{c}. In certain of these embodiments, R⁶ is C₃₋₈ cycloalkyl optionally substituted with from 1-2 R^{c}

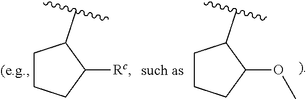

In certain embodiments (when R⁶ is R^{g}), R⁶ is heteroaryl including from 5-10 (e.g., 5-6) ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}. In certain of these embodiments, R⁶ is heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-2 R^{c}. For example, R⁶ can be

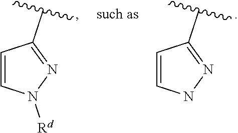

As another non-limiting example R⁶ can be

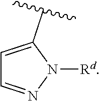

In certain embodiments, R⁶ is heteroaryl including 6 ring atoms, wherein from 1-4 ring atoms are ring nitrogen atoms, and wherein the heteroaryl is optionally substituted with from 1-4 R^{c}. For example, R⁶ can be

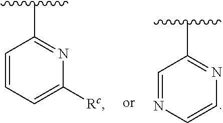

In certain embodiments, R⁶ is —R^{g2}—R^{W} or —R^{g2}—R^{Y}.

In certain of these embodiments, R⁶ is —R^{g2}—R^{W}.

In certain of the foregoing embodiments, —R⁶ is

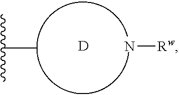

wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}.

In certain of the foregoing embodiments, —R⁶ is

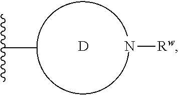

wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}; optionally wherein —R⁶ is a monocyclic heterocyclylene ring including from 3-10 ring atoms as defined above with a nitrogen atom bonded to R^{W}

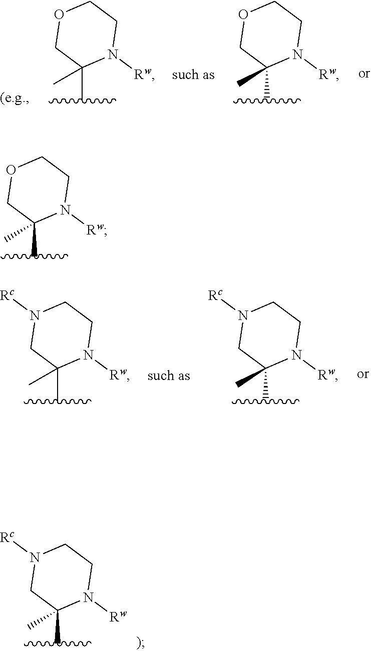

optionally wherein —R⁶ is a bicyclic heterocyclylene ring including from 3-10 ring atoms as defined above with a nitrogen atom bonded to R^{W}

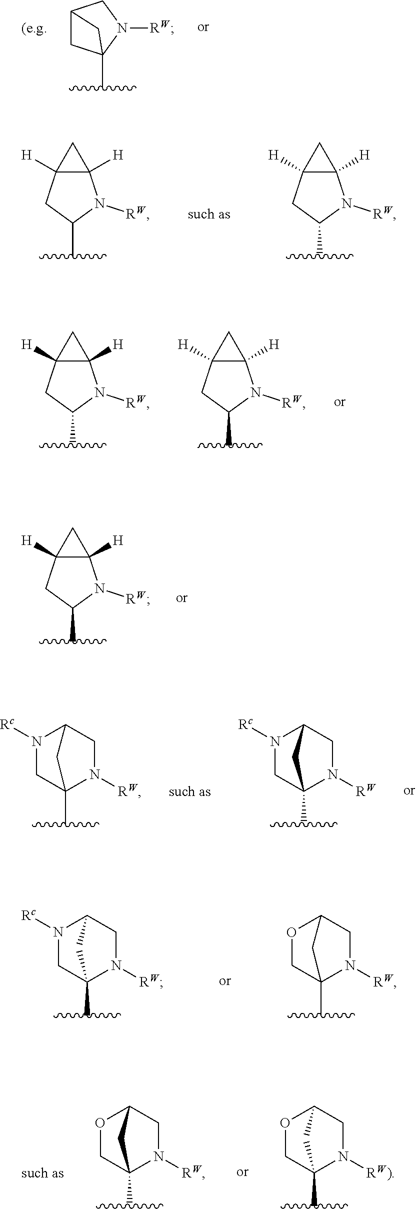

In certain of these embodiments, —R⁶ is

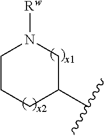

optionally substituted with from 1-2 R^{c}, wherein x1 and x2 are each independently 0, 1, or 2.

In certain embodiments, x1=0, and x2=0. In certain embodiments, x1=0, and x2=1. In certain embodiments, x1=0, and x2=2.

In certain embodiments, x1=0, and x2=0; or x1=0, and x2=1; or x1=0, and x2=2.

As non-limiting examples when R⁶ is

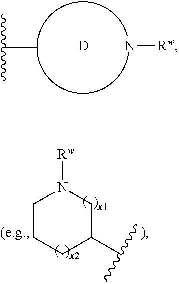

R⁶ can be selected from the group consisting of:

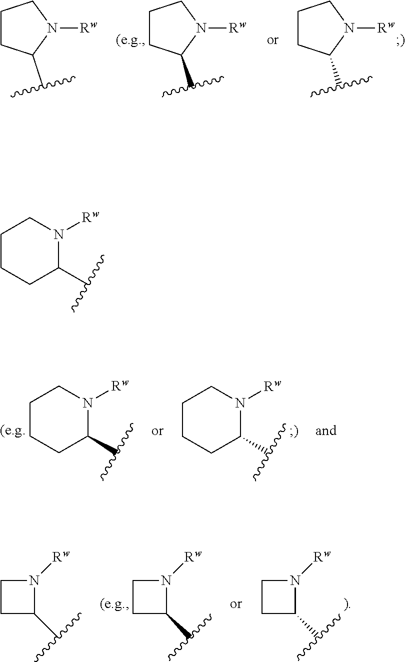

As non-limiting examples when R⁶ is

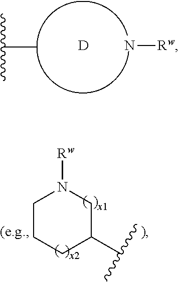

R⁶ can be selected from the group consisting of:

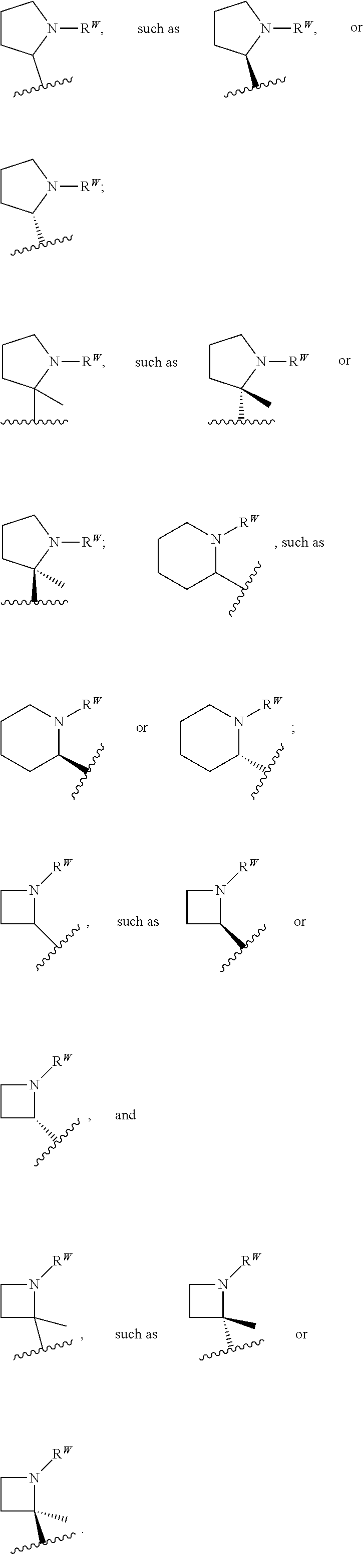

In certain embodiments, R⁶ is C₃-C₆ cycloalkyl (e.g. cyclobutyl) substituted with R^{W}; or oxetanyl substituted with R^{W}; or tetrahydrofuryl substituted with R^{W}.

In certain embodiments, R⁶ is —R^{W}

In certain embodiments (when R⁶ is —R^{g2}—R^{W}), —R^{W} is -L^{W}-W; and L^{W} is C(═O).

In certain embodiments, (when R⁶ is —R^{g2}—R^{W}, or when R⁶ is R^{W}), —R^{W} is -L^{W}. W; and L^{W} is C(═O) NHC(═O)*, NR^{d}C(═O)* (e.g., NMeC(═O)*), or NHS(O)₁₋₂* wherein the asterisk represents point of attachment to W.

In certain of these embodiments, W is C₂₋₆ alkenyl optionally substituted with from 1-3 R^{a} and further optionally substituted with R^{g}, wherein W is attached to L^{W} via an sp² hybridized carbon atom.

In certain of these embodiments, W is C₂₋₆ alkenyl or C₂₋₆ optionally substituted with from 1-3 R^{a} and further optionally substituted with R^{g}, wherein W is attached to L^{W} via an sp² or sp hybridized carbon atom.

In certain of the foregoing embodiments, W is C₂₋₄ alkenyl optionally substituted with from 1-3 R^{a} and further optionally substituted with R^{g}, wherein W is attached to L^{W} via an sp² hybridized carbon atom. For example, W can be CH═CH₂.

In certain of the foregoing embodiments, W is C₂₋₄ alkenyl (e.g., CH═CH₂) or C₂₋₄ alkynyl alkynyl

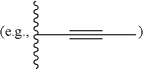 [SMILES: *C#CC]

optionally substituted with from 1-3 R^{a} and further optionally substituted with R^{g}, wherein W is attached to L^{W} via an sp² or sp hybridized carbon atom.

In certain embodiments, -L^{W}-W is —C(═O)CH═CH₂.

In certain embodiments, -L^{W}-W is —C(═O)CH═CH₂; —C(═O)NHCH═CH₂; C(═O)CH═CHCH₂NRR (e.g., C(═O)CH═CHCH₂N(HMe), C(═O)CH═CHCH₂NMe₂,

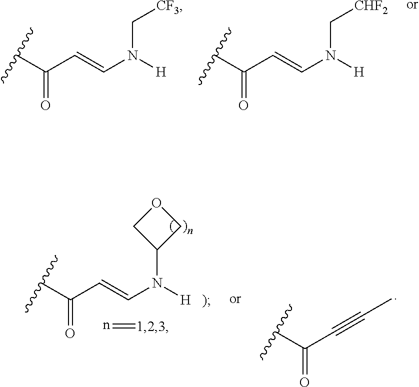

In certain embodiments, R⁶ is —C₁₋₆ alkoxy or —S(O)₀₋₂(C₁₋₆ alkyl), each optionally substituted with from 1-6 R^{a}.

In certain of these embodiments, R⁶ is —C₁₋₆ alkoxy. For example, R⁶ can be —C₁₋₃ alkoxy (e.g., methoxy).

In certain embodiments, L² is a bond.

In certain embodiments, L² is C₁₋₁₀ alkylene optionally substituted with from 1-6 R^{a}.

In certain embodiments, L² is C₁₋₁₀ alkylene optionally substituted with from 1-6 R^{a} wherein R^{a} is —NR^{e}R^{f} (e.g., NMe₂), halo (e.g., fluoro), alkoxyl (e.g., methoxy).

In certain of these embodiments, L² is C₁₋₆ alkylene optionally substituted with from 1-6 R^{a}. In certain of the foregoing embodiments, L² is branched C₃₋₆ alkylene. As a non-limiting example of the foregoing embodiments, L² can be

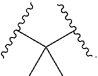 [SMILES: *C(*)(C)C]

In certain embodiments, L² is C₁₋₆ alkylene optionally substituted with from 1-6 R^{a}, wherein R^{a} is —NR^{e}R^{f} (e.g., NMe₂), halo (e.g., fluoro), alkoxyl (e.g., methoxy). In certain of these foregoing embodiments, L² is branched C₃₋₆ alkylene optionally substituted with from 1-6 R^{a}, wherein R^{a} is —NR^{e}R^{f} (e.g., NMe₂), halo (e.g., fluoro), alkoxyl (e.g., methoxy).

As non-limiting examples of the foregoing embodiments, L² can be

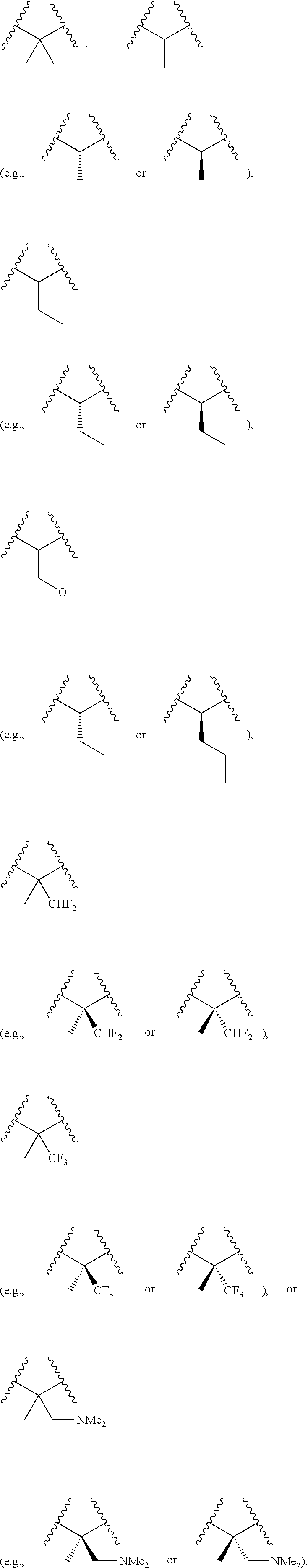

Variables R^{1c}, R^{2a}, R^{2b}, R^{3a}, and R^{3b}

In some embodiments, R^{1c} is H.

In some embodiments, R^{2a} and R^{2b} are H.

In some embodiments, from 1-2 (e.g., 1) of R^{2a} and R^{2b} is a substituent other than H.

In certain of these embodiments, one of R^{2a} and R^{2b} is C₁₋₃ alkyl optionally substituted with from 1-3 R^{a} (e.g., C₁₋₃ alkyl); and the other of R^{2a} and R^{2b} is H.

In some embodiments, R^{3a} and R^{3b} are H.

In some embodiments, from 1-2 (e.g., 1) of R^{3a} and R^{3b} is a substituent other than H.

In certain of these embodiments, one of R^{3a} and R^{3b} is C₁₋₃ alkyl optionally substituted with from 1-3 R^{a} (e.g., C₁₋₃ alkyl optionally substituted with from 1-3 —F); and the other of R^{2a} and R^{2b} is H.

In some embodiments, R^{3a} and R^{3b}, together with the Ring B ring atom to which each is attached, form a fused saturated or unsaturated ring of 3-12 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W}.

In certain embodiments, R^{3a} and R^{3b}, together with the Ring B ring atom to which each is attached, form a fused saturated ring of 4-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-8 ring atoms is optionally substituted with from 1-4 substituents independently selected from the group consisting of oxo, R^{c}, and R^{W}.

In certain of these embodiments, R^{3a} and R^{3b}, together with the Ring B ring atom to which each is attached, form:

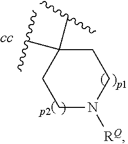

which is optionally substituted with from 1-2 substituents independently selected from the group consisting of oxo and R^{c}, wherein:
p1 and p2 are independently 0, 1, or 2;
R^{Q} is H, R^{d}, C(═O)—W, or S(O)₂W; and
cc represents the point of attachment to C(R^{2a}R^{2b})

In certain of these embodiments, R^{Q} is H. In certain embodiments, R^{Q} is R^{d}. In certain embodiments, R^{Q} is C₁₋₆ alkyl optionally substituted with from 1-3 independently selected R^{a}. In certain embodiments, R^{Q} is C(═O)—W or S(O)₂W. In certain of these embodiments, W is C₂₋₄ alkenyl. For example, R^{Q} can be C(═O)—CH₂═CH₂.

In certain of these embodiments, R^{3a} and R^{3b}, together with the Ring B ring atom to which each is attached, form

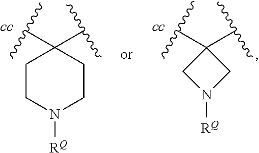

wherein R^{Q} is H, R^{d}, C(═O)—W, or S(O)₂W; and cc represents the point of attachment to C(R^{2a}R^{2b}). In certain of these embodiments, R^{Q} is H. In certain embodiments, R^{Q} is R^{d}. In certain embodiments, R^{Q} is C₁₋₆ alkyl optionally substituted with from 1-3 independently selected R^{a}. In certain embodiments, R^{Q} is C(═O)—W or S(O)₂W. In certain of these embodiments, W is C₂₋₄ alkenyl. For example, R^{Q} can be C(═O)—CH₂═CH₂.

In certain embodiments, R^{3a} and R^{3b}, together with the Ring B ring atom to which each is attached, form a fused ring selected from the group consisting of:

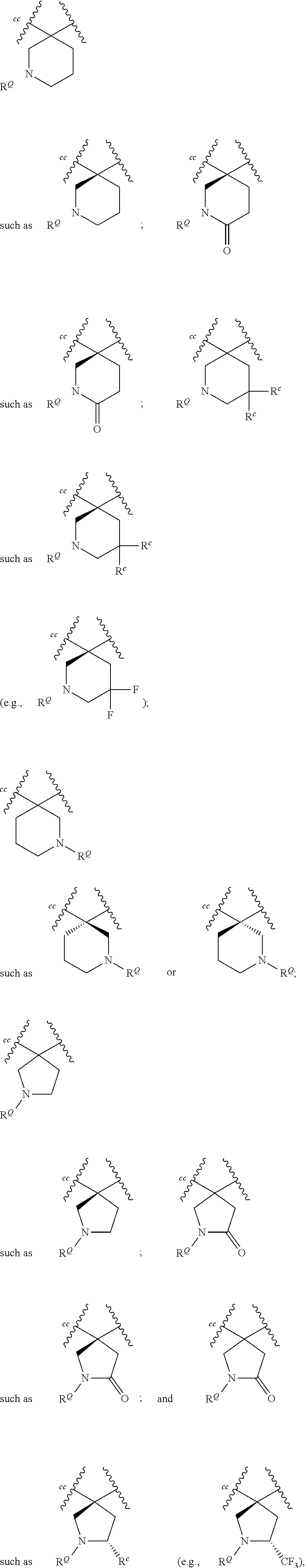

wherein R^{Q} is H, R^{d}, C(═O)—W, or S(O)₂W; and cc represents the point of attachment to C(R^{2a}R^{2b}). In certain of these embodiments, R^{Q} is H. In certain embodiments, R^{Q} is R^{d}. In certain embodiments, R^{Q} is C₁₋₆ alkyl optionally substituted with from 1-3 independently selected R^{a}. In certain embodiments, R^{Q} is C(═O)—W or S(O)₂W. In certain of these embodiments, W is C₂₋₄ alkenyl. For example, R^{Q} can be C(═O)—CH₂═CH₂.

### Variable Ring A

In some embodiments, Ring A is

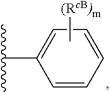

wherein each R^{cB} is an independently selected R^{c}; and m is 0, 1, 2, 3, or 4.

In certain of these embodiments, m is 1, 2, or 3. For example, m can be 1 or 2 (e.g., 2).

In certain embodiments, Ring A is

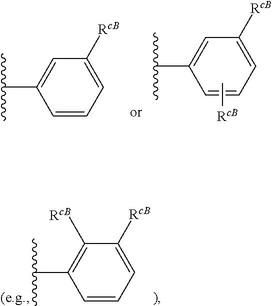

wherein each R^{cB} is an independently selected R^{c}.

In certain embodiments, each R^{cB} is independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

In certain embodiments, Ring A is

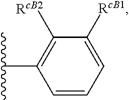

wherein R^{cB1} is R^{c}; and R^{cB2} is H or R^{c}.

In certain of these embodiments, R^{cB}1 is halo (e.g., —F or —Cl (e.g., —F)).

In certain embodiments, R^{cB2} is C₁₋₄ alkoxy or C₁₋₄ haloalkoxy (e.g., C₁₋₄ alkoxy (e.g., methoxy)).

As non-limiting examples of the foregoing embodiments, Ring A can be

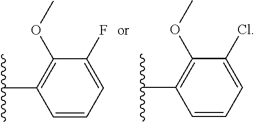

In certain embodiments, Ring A is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

In certain of these embodiments, Ring A is bicyclic heteroaryl including from 9-ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

As non-limiting examples of the foregoing embodiments, Ring A can be quinolinyl, indazolyl, pyrazolopyridyl, or isothiazolopyridyl, each of which is optionally substituted with from 1-2 R^{c}, wherein a ring nitrogen is optionally substituted with R^{d}. For example, Ring A can be:

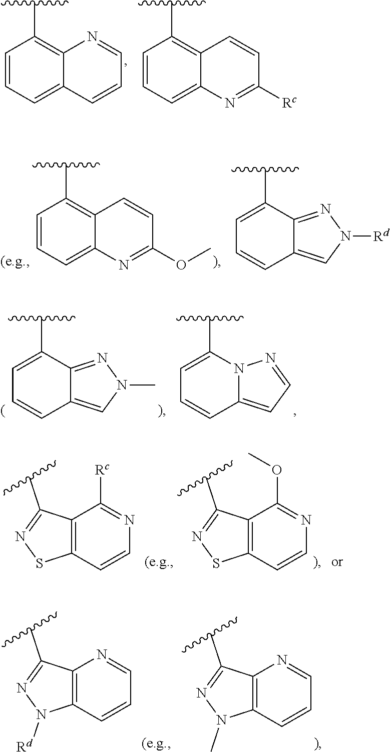

each of which is further optionally substituted with R^{c}. As another non-limiting example, Ring A can be

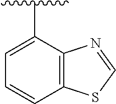 [SMILES: *c1cccc2scnc12]

which is further optionally substituted with R^{c}.

Variables n, R⁷, and R⁴

In some embodiments, n is 0. In some embodiments, n is 1 or 2. In certain of these embodiments, n is 1. In certain embodiments, one occurrence of R⁷ is NR^{e}R^{f} (e.g., NH₂, NH(C₁₋₃ alkyl), or N(C₁₋₃ alkyl)₂). In certain of these embodiments, one occurrence of R⁷ is NH₂ or NH(C₁₋₃ alkyl). For example, one occurrence of R⁷ can be NH₂.

In certain embodiments, the

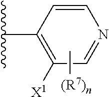

moiety is

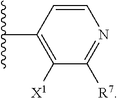

In certain of these embodiments, one occurrence of R⁷ is NR^{e}R^{f} (e.g., NH₂, NH(C₁₋₃ alkyl), or N(C₁₋₃ alkyl)₂). In certain of the foregoing embodiments, one occurrence of R⁷ is NH₂ or NH(C₁₋₃ alkyl). For example, one occurrence of R⁷ can be NH₂.

In some embodiments, R⁴ is H.

### Non-Limiting Combinations

In certain embodiments, the compound of Formula (I) is a compound of Formula (I-a)

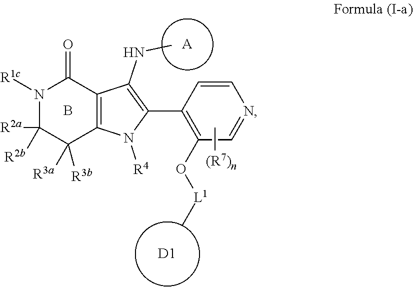

or a pharmaceutically acceptable salt thereof,

wherein Ring D1 is selected from the group consisting of:
monocyclic heteroaryl including from 5-6 ring atoms, wherein from 1-4 ring atoms are heteroatoms, each independently selected from the group consisting of N, N(H), N(R^{d}), O, and S, and wherein the heteroaryl is optionally substituted with from 1-4 R^{cA}; and
—R^{g2}—R^{Y}, wherein the —R^{g2} present in —R^{g2}—R is monocyclic heteroarylene including from 5-6 ring atoms, wherein from 1-4 ring atoms are heteroatoms, each independently selected from the group consisting of N, N(H), N(R^{d}), O, and S, and wherein the heteroarylene is optionally substituted with from 1-3 R^{cA},

wherein each R^{cA} is an independently selected R^{c}; and

L¹ is a bond or C₁₋₃ alkylene optionally substituted with from 1-6 R^{a}.

In certain embodiments of Formula (I-a), Ring D1 is monocyclic heteroaryl including 5 ring atoms, wherein from 1-4 ring atoms are heteroatoms, each independently selected from the group consisting of N, N(H), N(R^{d}), O, and S, and wherein the heteroaryl is optionally substituted with from 1-4 R^{cA}.

As non-limiting examples of the foregoing embodiments, Ring D1 can be selected from the group consisting of:

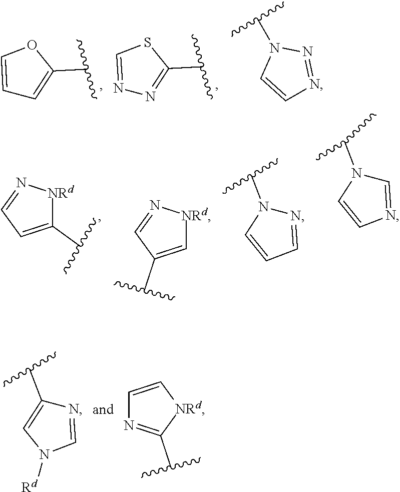

each optionally substituted with from 1-2 R^{cA}.

As non-limiting examples of the foregoing embodiments, Ring D1 can be selected from the group consisting of:

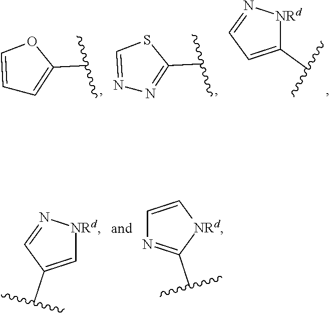

each optionally substituted with from 1-2 R^{cA}.

In certain embodiments of Formula (I-a), Ring D1 monocyclic heteroaryl including 6 ring atoms, wherein from 1-4 ring atoms are ring nitrogen atoms, and wherein the heteroaryl is optionally substituted with from 1-4 R^{cA}.

As non-limiting examples of the foregoing embodiments, Ring D1 can be selected from the group consisting of:

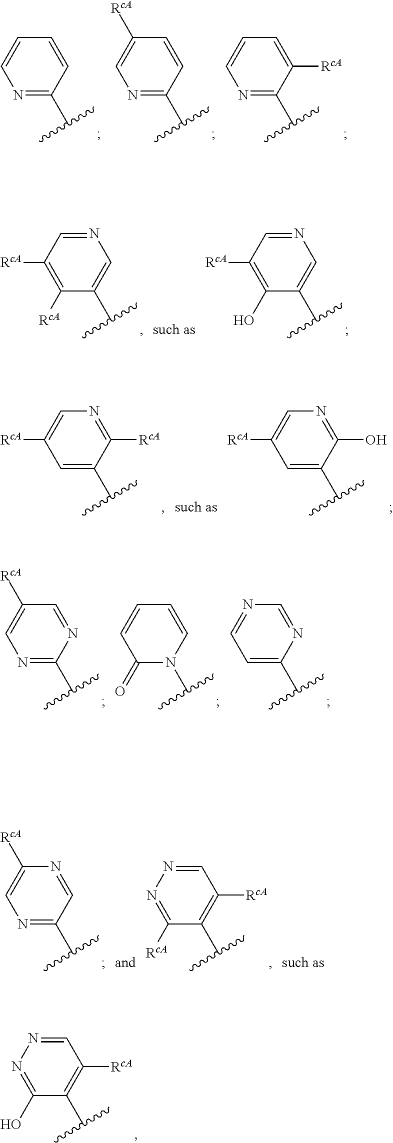

each of which is further optionally substituted with R^{cA}.

As non-limiting examples of the foregoing embodiments, Ring D1 can be selected from the group consisting of:

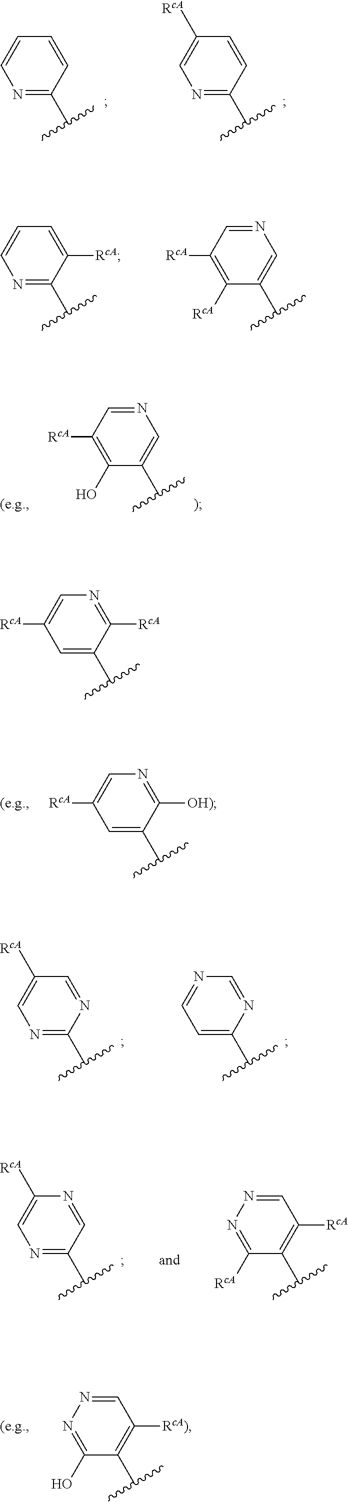

each of which is further optionally substituted with R^{cA}.

In certain embodiments of Formula (I-a), Ring D1 is —R^{g2}—R^{Y}; and the —R^{g2} present in —R^{g2}—R is monocyclic heteroarylene including from 5-6 ring atoms, wherein from 1-4 ring atoms are heteroatoms, each independently selected from the group consisting of N, N(H), N(R^{d}), O, and S, and wherein the heteroarylene is optionally substituted with from 1-3 R^{cA}.

In certain of these embodiments, Ring D1 is —R^{g2}—R^{Y}; and the —R^{g2} present in —R^{g2}—R is monocyclic heteroarylene including 5 ring atoms, wherein from 1-4 ring atoms are heteroatoms, each independently selected from the group consisting of N, N(H), N(R^{d}), O, and S, and wherein the heteroarylene is optionally substituted with from 1-2 R^{cA}. For example, Ring D1 can be

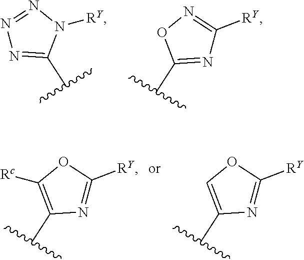

In certain embodiments of Formula (I-a) (when Ring D1 is —R^{g2}—R^{Y}), R^{Y} is selected from the group consisting of:
phenyl optionally substituted with from 1-3 R^{e}; and
monocyclic heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

In certain embodiments of Formula (I-a), n is 0.

In certain embodiments of Formula (I-a), n is 1 or 2. For example, n can be 1.

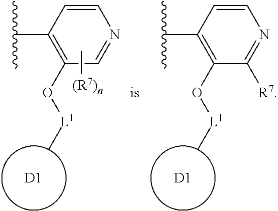

In certain embodiments of Formula (I-a), is.

In certain embodiments of Formula (I-a), R⁷ is NR^{e}R^{f}, such as NH₂, NH(C₁₋₃ alkyl), or N(C₁₋₃ alkyl)₂, such as wherein R⁷ is NH₂.

In certain embodiments, the compound of Formula (I) is a compound of Formula (I-b)

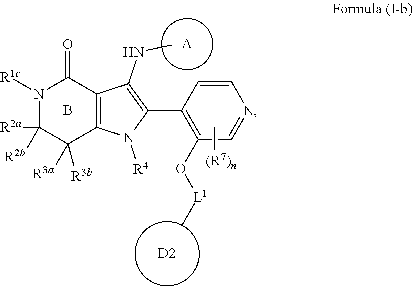

or a pharmaceutically acceptable salt thereof,

wherein Ring D2 is bicyclic heteroaryl including from 8-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}; and

L¹ is a bond or C₁₋₃ alkylene optionally substituted with from 1-6 R^{a}.

In certain embodiments of Formula (I-b), Ring D2 is heteroaryl including 8 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

As non-limiting examples of the foregoing embodiments, Ring D2 can be selected from the group consisting of:

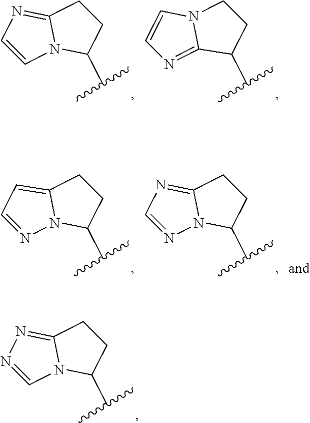

each of which is further optionally substituted with from 1-2 R^{cA}, wherein each R^{cA} is an independently selected R^{c}.

As further non-limiting examples, Ring D2 can be selected from the group

consisting of

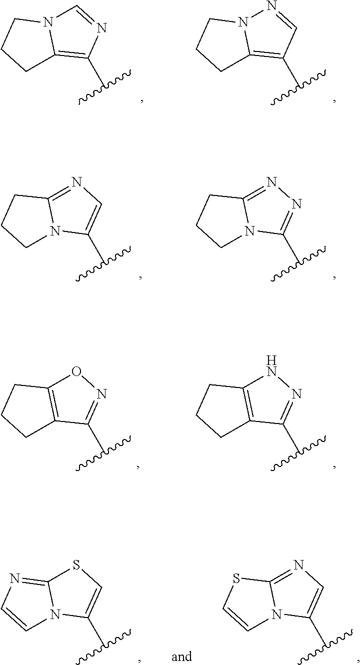

each of which is further optionally substituted with from 1-2 R^{cA}, wherein each R^{cA} is an independently selected R^{c}.

In certain embodiments of Formula (I-b), Ring D2 is bicyclic heteroaryl including 9 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

In certain of these embodiments, Ring D2 is imidazolopyridinyl, pyrazolopyridinyl, or benzotriazolyl, each of which is optionally substituted with from 1-2 R^{cA}, wherein each R^{cA} is an independently selected R^{c}.

As non-limiting examples of the foregoing embodiments, Ring D2 can be

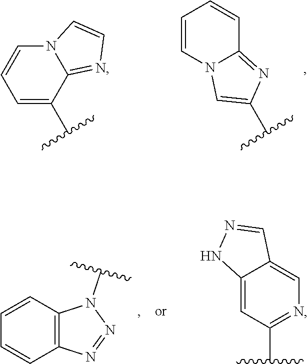 [SMILES: *c1cccn2ccnc12 ; *c1cn2ccccc2n1 ; *n1nnc2ccccc21 ; *c1cc2[nH]ncc2cn1]

each of which is optionally substituted with from 1-2 R^{cA}, wherein each R^{cA} is an independently selected R^{c}.

In certain embodiments of Formula (I-b), n is 0.

In certain embodiments of Formula (I-b), n is 1 or 2. For example, n can be 1.

In certain embodiments of Formula (I-b),

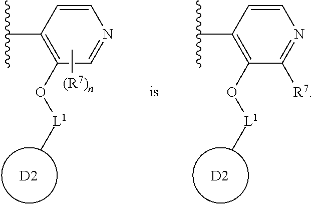

In certain embodiments of Formula (I-b), R⁷ is NR^{e}R^{f}, such as NH₂, NH(C₁₋₃ alkyl), or N(C₁₋₃ alkyl)₂, such as wherein R⁷ is NH₂.

In certain embodiments of Formula (I-a) or (I-b), each R^{cA} is independently selected from the group consisting of: halo; cyano; —OH; C₁₋₆ alkyl which is optionally substituted with from 1-6 independently selected R^{a}; C₁₋₄ alkoxy optionally substituted with C₁₋₄ alkoxy or C₁₋₄ haloalkoxy; C₁₋₄ haloalkoxy; and —C(═O)NR′R″.

In certain embodiments, one occurrence of R^{cA} is an independently selected halo, such as —F or —Cl. In certain embodiments, one occurrence of R^{cA} is cyano. In certain embodiments, one occurrence of R^{cA} is C₁₋₆ alkyl which is optionally substituted with from 1-6 independently selected R^{a}. In certain embodiments, one occurrence of R^{cA} is C₁₋₆ alkyl, such as C₁₋₃ alkyl. In certain embodiments, one occurrence of R^{cA} is C₁₋₆ alkyl substituted with —OH or —NR^{e}R^{f}. For example, one occurrence of R^{cA} can be C₁₋₃ alkyl substituted with —OH or NH₂. In certain embodiments, one occurrence of R^{cA} is C₁₋₄ alkoxy optionally substituted with C₁₋₄ alkoxy or C₁₋₄ haloalkoxy. For example, one occurrence of R^{cA} can be C₁₋₄ alkoxy (e.g., methoxy or ethoxy).

In certain embodiments, the compound of Formula (I) is a compound of Formula (I-c)

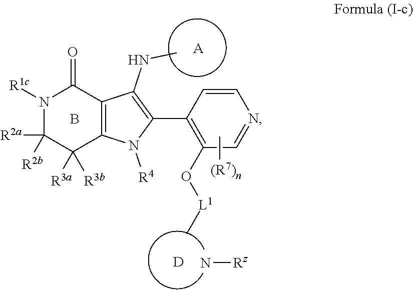

or a pharmaceutically acceptable salt thereof,

wherein Ring D is heterocyclylene including from 3-10 ring atoms, wherein from 0-2 ring atoms (in addition to the ring nitrogen atom bonded to R^{Z}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c};
R^{Z} is R^{X} or R^{Y}; and
L¹ is a bond or C₁₋₃ alkylene optionally substituted with from 1-6 R^{a}.

In certain embodiments of Formula (I-c), R^{Z} is R^{X}.

In certain of these embodiments, R^{Z} is C(═O)(C₁₋₄ alkyl).

In certain embodiments, R^{Z} is S(O)₂(C₁₋₄ alkyl).

In certain embodiments of Formula (I-b), R^{Z} is R^{Y}.

In certain of these embodiments, R^{Z} is R^{g}

In certain of the foregoing embodiments, R^{Z} is selected from the group consisting of:
phenyl optionally substituted with from 1-3 R^{c}; and
monocyclic heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

In certain embodiments of Formula (I-c), n is 0.

In certain embodiments of Formula (I-c), n is 1 or 2, such as wherein n is 1.

In certain embodiments of Formula (I-c),

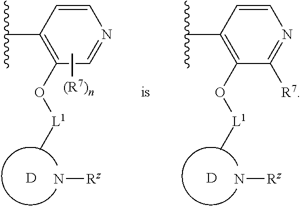

In certain embodiments of Formula (I-c), R⁷ is NR^{e}R^{f}, such as NH₂, NH(C₁₋₃ alkyl), or N(C₁₋₃ alkyl)₂, such as wherein R⁷ is NH₂.

In certain embodiments, the compound of Formula (I) is a compound of Formula (I-d)

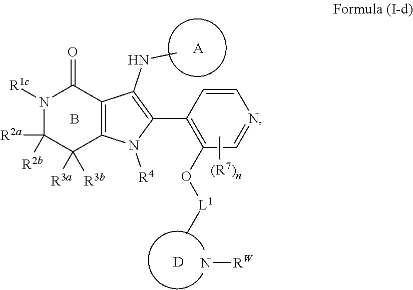

or a pharmaceutically acceptable salt thereof,

wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}; and
L¹ is a bond or C₁₋₃ alkylene optionally substituted with from 1-6 R^{a}.

In certain embodiments of Formula (I-d), R^{W} is -L^{W}-W; and L^{W} is C(═O).

In certain of these embodiments, W is C₂₋₆ alkenyl optionally substituted with from 1-3 R^{a} and further optionally substituted with R^{g}, wherein W is attached to L^{W} via an sp² hybridized carbon atom. For example, W can be CH═CH₂.

In certain of these embodiments, W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom. As non-limiting examples, W can be CH═CH₂, CH═CHCH₂NMe₂, or

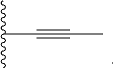 [SMILES: *C#CC]

In certain embodiments of Formula (I-c) or (I-d), Ring D is

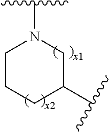

which is optionally substituted with from 1-2 R^{c}, wherein x1 and x2 are each independently 0, 1, or 2. In certain of these embodiments, x1 is 0.

As non-limiting examples of the foregoing embodiments, Ring D can be selected from the group consisting of:

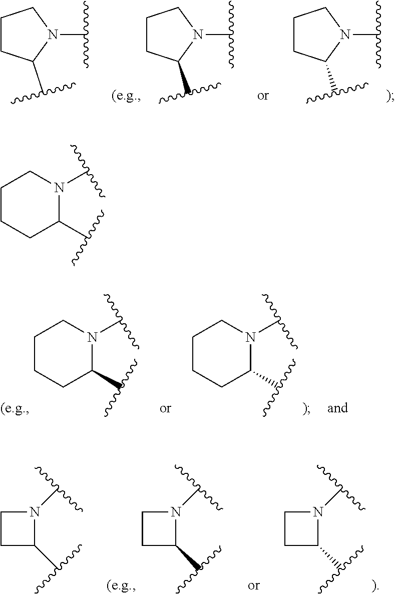

As further non-limiting examples, of the foregoing embodiments, Ring D can be selected from the group consisting of:

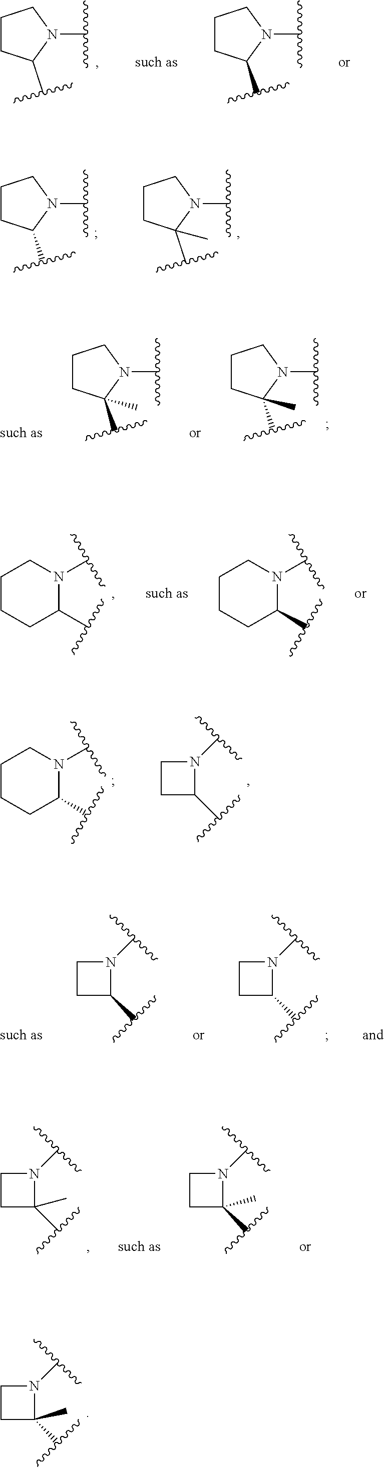

In certain embodiments of Formula (I-d), n is 0.

In certain embodiments of Formula (I-d), n is 1 or 2. For example, n can be 1.

In certain embodiments of Formula (I-d),

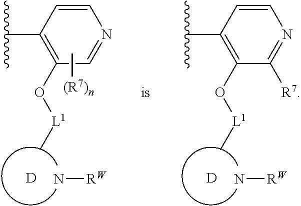

In certain embodiments of Formula (I-d),

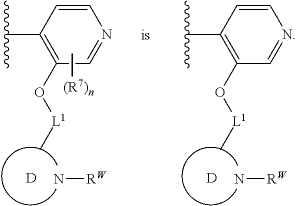

In certain embodiments of Formula (I-d), R⁷ is NR^{e}R^{f}, such as NH₂, NH(C₁₋₃ alkyl), or N(C₁₋₃ alkyl)₂, such as wherein R⁷ is NH₂.

In certain embodiments, the compound of Formula (I) is a compound of Formula (I-e):

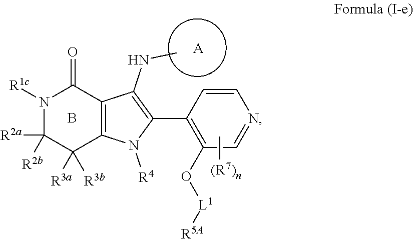

or a pharmaceutically acceptable salt thereof,

wherein R^{5A} is -L⁵-R^{g} or —S(O)₀₋₂(C₁₋₆ alkyl) which is optionally substituted with from 1-6 R^{a}; and
L¹ is C₁₋₁₀ alkylene optionally substituted with from 1-6 R^{a}.

In certain embodiments of Formula (I-e), R^{5A} is -L⁵-R^{g}. In certain of these embodiments, R^{5A} is —O—R^{g}.

In certain of the foregoing embodiments, R^{5A} is —O—R^{g}; and the R^{g} present in —O—R^{g} is C₃₋₁₀ cycloalkyl or C₃₋₁₀ cycloalkenyl, each optionally substituted with from 1-4 substituents each independently selected from the group consisting of: oxo and R^{c}.

In certain embodiments, R^{5A} is —O—(C₃₋₆ cycloalkyl), wherein the C₃₋₆ cycloalkyl is optionally substituted with from 1-3 R^{c}. For example, R⁵ can be

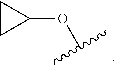 [SMILES: *OC1CC1]

In certain embodiments of Formula (I-e), R^{5A} is —S(O)₀₋₂(C₁₋₆ alkyl) which is optionally substituted with from 1-6 R^{a}. In certain of these embodiments, R^{5A} is —S(O)₂(C₁₋₆ alkyl) which is optionally substituted with from 1-6 R^{a}. As non-limiting examples of the foregoing embodiments, R^{5A} can be —S(O)₂(C₁₋₃ alkyl) (e.g., —S(O)₂Me).

In certain embodiments of Formula (I-e), n is 0.

In certain embodiments of Formula (I-e), n is 1 or 2, such as wherein n is 1.

In certain embodiments of Formula (I-e),

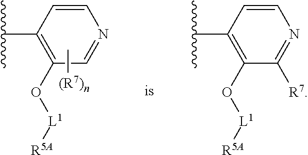

In certain embodiments of Formula (I-e), R⁷ is NR^{e}R^{f}, such as NH₂, NH(C₁₋₃ alkyl), or N(C₁₋₃ alkyl)₂, such as wherein R⁷ is NH₂.

In certain embodiments of Formula (I-a), (I-b), (I-c), (I-d), or (I-e), L¹ is C₁₋₃ alkylene optionally substituted with from 1-6 R^{a}.

In certain of these embodiments, L¹ is C₁₋₃ alkylene. For example, L¹ can be —CH₂—. As another non-limiting example, L¹ can be —CH₂CH₂—.

In certain embodiments of Formula (I-a), (I-b), (I-c), (I-d), or (I-e), L¹ is a bond.

In certain embodiments, the compound of Formula (I) is a compound of Formula (I-f):

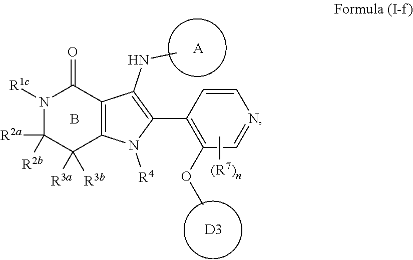

or a pharmaceutically acceptable salt thereof,

wherein Ring D3 is C₃₋₁₀ cycloalkyl substituted with from 1-4 substituents each independently selected from the group consisting of: oxo and R^{c}.

In certain embodiments of Formula (I-f), Ring D3 is C₃₋₆ cycloalkyl substituted with C₁₋₄ alkoxy or C₁₋₄ haloalkoxy; and R⁵ is further optionally substituted from 1-2 substituents each independently selected from the group consisting of: oxo and R^{c}.

In certain of these embodiments, R⁵ is cyclopropyl that is substituted with C₁₋₄ alkoxy or C₁₋₄ haloalkoxy. For example, R⁵ can be:

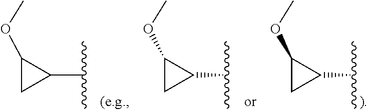

In certain embodiments of Formula (I-f), n is 0.

In certain embodiments of Formula (I-f), n is 1 or 2, such as wherein n is 1.

In certain embodiments of Formula (I-f),

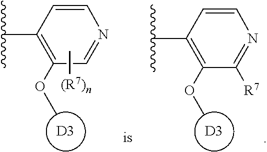

In certain embodiments of Formula (I-f), R⁷ is NR^{e}R^{f}, such as NH₂, NH(C₁₋₃ alkyl), or N(C₁₋₃ alkyl)₂, such as wherein R⁷ is NH₂.

In certain of these embodiments, the compound of Formula (I) is a compound of Formula (I-g):

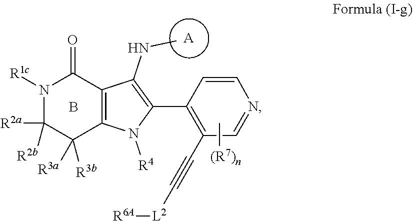

or a pharmaceutically acceptable salt thereof,

wherein L² is C₁₋₆ alkylene optionally substituted with from 1-6 R^{a}; and
R^{6A} is selected from the group consisting of —C₁₋₆ alkoxy optionally substituted with from 1-6 R^{a}; NR^{e}R^{f}; H; halo; and —OH.

In certain embodiments of Formula (I-g), R^{6A} is —C₁₋₆ alkoxy optionally substituted with from 1-6 R^{a}.

In certain of these embodiments, R^{6A} is —C₁₋₃ alkoxy (e.g., methoxy).

In certain embodiments, R^{6A} is NR^{e}R^{f}.

In certain embodiments, R^{6A} is H, halo, or —OH.

In certain embodiments of Formula (I-g), L² is branched C₃₋₆ alkylene. As a non-limiting example of the foregoing embodiments, L² can be

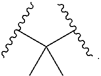 [SMILES: *C(*)(C)C]

In certain embodiments of Formula (I-g), L² is C₁₋₃ alkylene, such as —CH₂—.

In certain embodiments of Formula (I-g), n is 0.

In certain embodiments of Formula (I-g), n is 1 or 2, such as wherein n is 1.

In certain embodiments of Formula (I-g),

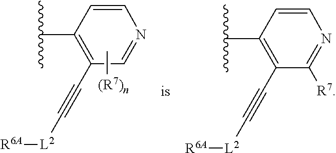

In certain embodiments of Formula (I-g), R⁷ is NR^{e}R^{f}, such as NH₂, NH(C₁₋₃ alkyl), or N(C₁₋₃ alkyl)₂, such as wherein R⁷ is NH₂.

In certain embodiments, the compound of Formula (I) is a compound of Formula (I-h):

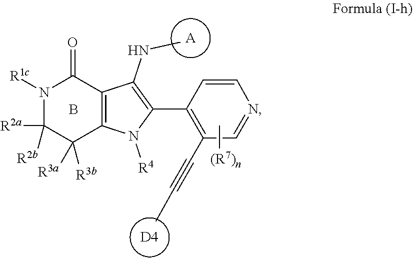

or a pharmaceutically acceptable salt thereof,

wherein Ring D4 is R^{g}.

In certain embodiments of Formula (I-h), Ring D4 is selected from the group consisting of:
C₃₋₁₀ cycloalkyl or C₃₋₁₀ cycloalkenyl, each of which is optionally substituted with from 1-4 substituents independently selected from the group consisting of oxo and R^{c}; and
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

In certain of these embodiments, Ring D4 is heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

In certain of the foregoing embodiments, Ring D4 is selected from the group consisting of pyrrolidinyl, piperidinyl, oxentanyl, tetrahydrofuranyl, and tetrahydropyranyl, each of which is optionally substituted with 1-2 substituents independently selected from the group consisting of oxo and R^{c}, wherein the ring nitrogen of the pyrrolidinyl or piperidinyl is optionally substituted with R^{d}. As non-limiting examples of the foregoing embodiments, Ring D4 can be:

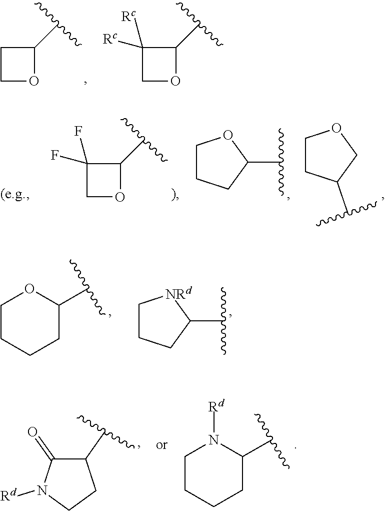

In certain of the foregoing embodiments, Ring D4 is selected from the group consisting of pyrrolidinyl, piperidinyl, tetrahydrofuranyl, and tetrahydropyranyl, each of which is optionally substituted with 1-2 substituents independently selected from the group consisting of oxo and R^{c}, wherein the ring nitrogen of the pyrrolidinyl or piperidinyl is optionally substituted with R^{d}. As non-limiting examples of the foregoing embodiments, Ring D4 can be:

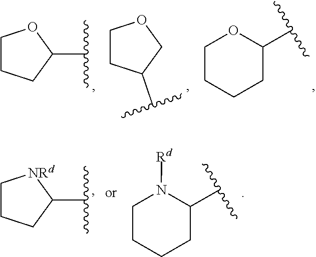

In certain embodiments of Formula (I-h), Ring D4 is heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}. For example, R⁶ can be

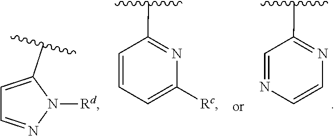

In certain embodiments of Formula (I-h), n is 0.

In certain embodiments of Formula (I-h), n is 1 or 2, such as wherein n is 1.

In certain embodiments of Formula (I-h),

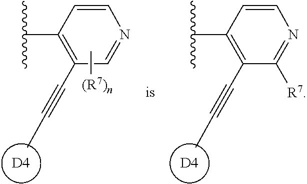

In certain embodiments of Formula (I-h), R⁷ is NR^{e}R^{f}, such as NH₂, NH(C₁₋₃ alkyl), or N(C₁₋₃ alkyl)₂, such as wherein R⁷ is NH₂.

In certain embodiments, the compound of Formula (I) is a compound of Formula (I-i)

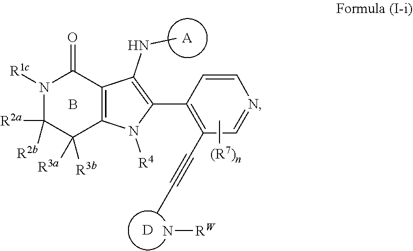

or a pharmaceutically acceptable salt thereof,

wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}.

In certain embodiments of Formula (I-i), R^{W} is -L^{W}-W; and L^{W} is C(═O).

In certain embodiments of Formula (I-i), W is C₂₋₆ alkenyl optionally substituted with from 1-3 R^{a} and further optionally substituted with R^{g}, wherein W is attached to L^{W} via an sp² hybridized carbon atom. As a non-limiting example of the foregoing embodiments, W can be CH═CH₂.

In certain embodiments of Formula (I-i), W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom. As non-limiting examples of the foregoing embodiments, W can be CH═CH₂, CH═CHCH₂NMe₂, or

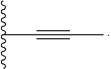 [SMILES: *C#CC]

In certain embodiments of Formula (I-i), -L^{W}-W is —C(═O)CH═CH₂; —C(═O)NHCH═CH₂; C(═O)CH═CHCH₂NRR (e.g., C(═O)CH═CHCH₂N(HMe), C(═O)CH═CHCH₂NMe₂,

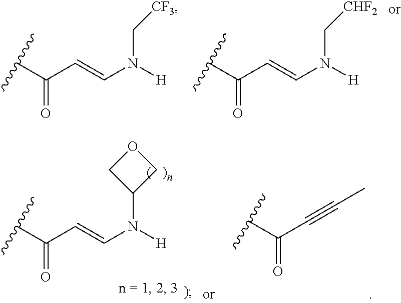

In certain embodiments of Formula (I-i), Ring D is

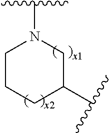

which is optionally substituted with from 1-2 R^{c}, wherein x1 and x2 are each independently 0, 1, or 2.

In certain of these embodiments, x1 is 0.

As non-limiting examples of the foregoing embodiments, Ring D can be selected from the group consisting of:

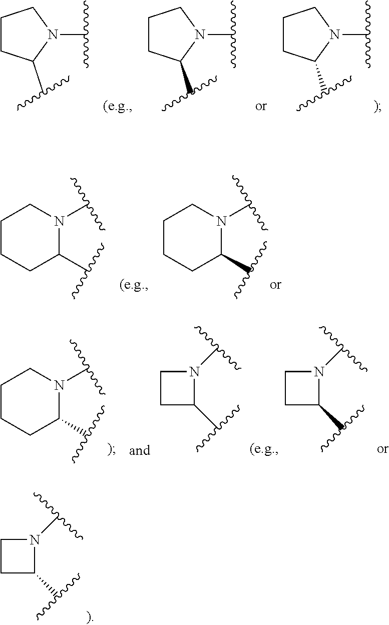

As further non-limiting examples of the foregoing embodiments, Ring D can be selected from the group consisting of:

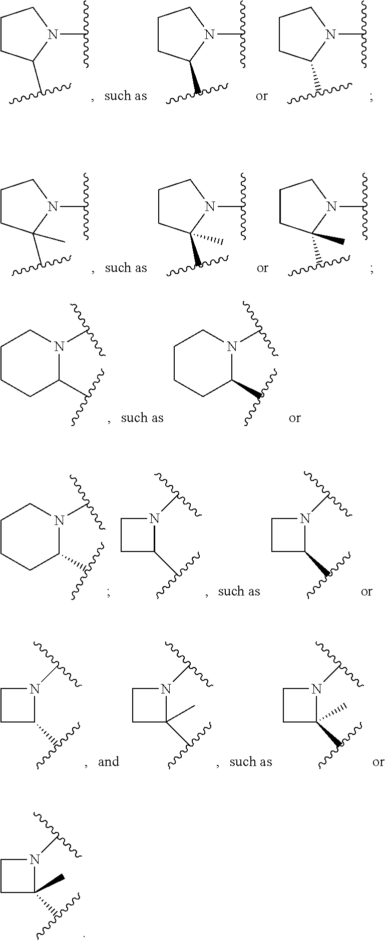

In certain embodiments of Formula (I-i),

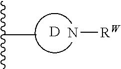

is heterocyclylene bonded to R^{W} including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}; optionally wherein

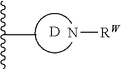

is a monocyclic heterocyclylene ring including from 3-10 ring atoms as defined above with a nitrogen atom bonded to R^{W}

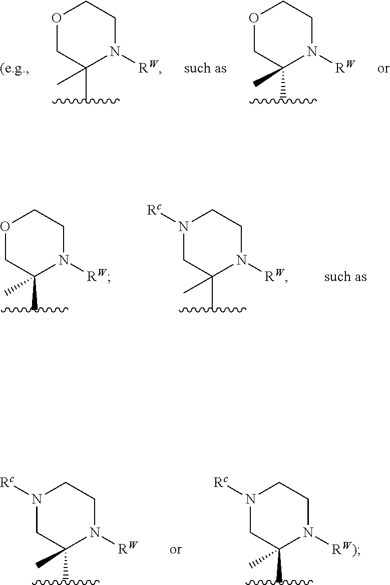

optionally wherein

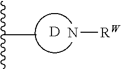

is a bicyclic heterocyclylene ring including from 3-10 ring atoms as defined above with a nitrogen atom bonded to R^{W} (e.g.

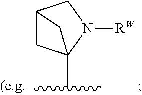

; or

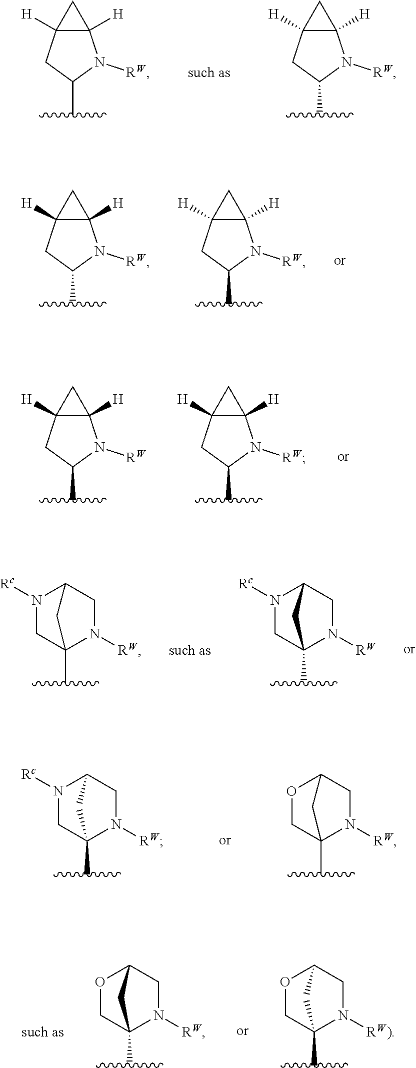

In certain embodiments of Formula (I-i), n is 0.

In certain embodiments of Formula (I-i), n is 1 or 2, such as wherein n is 1.

In certain embodiments of Formula (I-i),

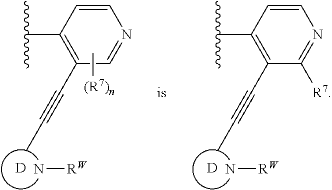

In certain embodiments of Formula (I-i),

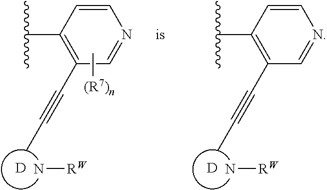

In certain embodiments of Formula (I-i), R⁷ is NR^{e}R^{f}, such as NH₂, NH(C₁₋₃ alkyl), or N(C₁₋₃ alkyl)₂, such as wherein R⁷ is NH₂.

In certain embodiments, the compound of Formula (I) is a compound of Formula (I-j)

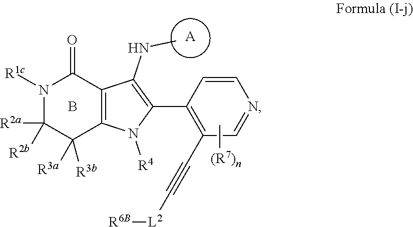

or a pharmaceutically acceptable salt thereof,

wherein L² is C₁₋₆ alkylene optionally substituted with from 1-6 R^{a}; and R⁶Bis —R^{W}.

In certain embodiments of Formula (I-j), R^{W} is -L^{W}-W; and L^{W} is C(═O), NHC(═O)*, or NHS(O)₁₋₂* wherein the asterisk represents point of attachment to W.

In certain embodiments of Formula (I-j), W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom. As a non-limiting example of the foregoing embodiments, W can be CH═CH₂, CH═CHCH₂NMe₂, or

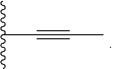 [SMILES: *C#CC]

In certain embodiments of Formula (I-j), -L^{W}-W is —C(═O)CH═CH₂, —C(═O)CH═CHCH₂NMe₂, or

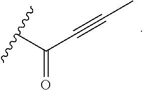 [SMILES: *C(=O)C#CC]

In certain embodiments of Formula (I-j), L² is C₁₋₃ alkylene optionally substituted with from 1-6 R^{a}, wherein R^{a} can be —NR^{e}R^{f} (e.g., Nme₂), halo (e.g., fluoro), or alkoxyl (e.g., methoxy).

As non-limiting examples of the foregoing embodiments of Formula (I-j), L² can be

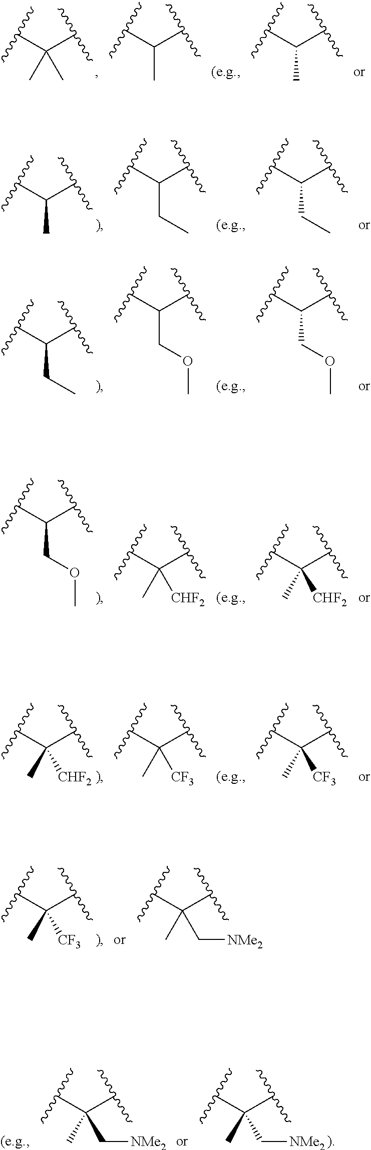

In certain embodiments of Formula (I-j), n is 0.

In certain embodiments of Formula (I-j), n is 1 or 2, such as wherein n is 1.

In certain embodiments of Formula (I-j),

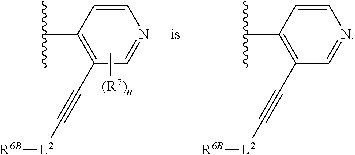

In certain embodiments of Formula (I-j),

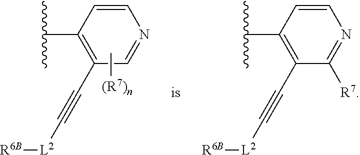

In certain embodiments of Formula (I-j), R⁷ is NR^{e}R^{f}, such as NH₂, NH(C₁₋₃ alkyl), or N(C₁₋₃ alkyl)₂, such as wherein R⁷ is NH₂.

In certain embodiments, the compound of Formula (I) is a compound of Formula (I-k):

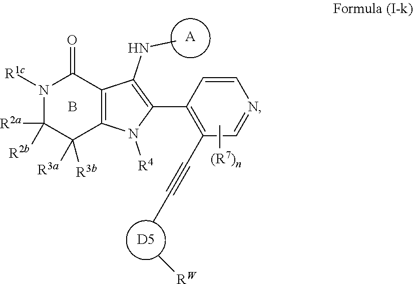

or a pharmaceutically acceptable salt thereof,

wherein Ring D5 is R^{g2}.

In certain embodiments of Formula (I-k), Ring D5 is selected from the group consisting of:
C₃₋₁₀ cycloalkylene or C₃₋₁₀ cycloalkenylene, each of which is optionally substituted with from 1-4 substituents independently selected from the group consisting of oxo and R^{c}; and
heterocyclylene or heterocycloalkenylene including from 3-10 ring atoms, wherein from 1-3 ring atoms are heteroatoms, each independently selected from the group consisting of N, N(H), N(R^{d}), O, and S(O)₀₋₂, and wherein the heterocyclylene or heterocycloalkenylene is optionally substituted with from 1-4 substituents independently selected from the group consisting of oxo and R^{c}.

In certain embodiments of Formula (I-k), Ring D5 is heterocyclylene including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

In certain embodiments of Formula (I-k), Ring D5 is C₃-C₆ cycloalkylene (e.g. cyclobutylene), oxetanylene, or tetrahydrofurylene.

In certain embodiments of Formula (I-k), R^{W} is -L^{W}-W; and L^{W} is C(═O) or NHC(═O)*, NR^{d}C(═O)*, NHS(O)₁₋₂*, wherein the asterisk represents point of attachment to W.

In certain embodiments of Formula (I-k), W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom. As non-limiting examples, W can be CH═CH₂, CH═CHCH₂NMe₂, or

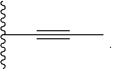 [SMILES: *C#CC]

In certain embodiments of Formula (I-k), -L^{W}-W is —C(═O)CH═CH₂, —C(═O)CH═CHCH₂NMe₂, or

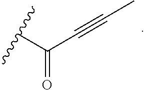 [SMILES: *C(=O)C#CC]

In certain embodiments of Formula (I-k), n is 0.

In certain embodiments of Formula (I-k),

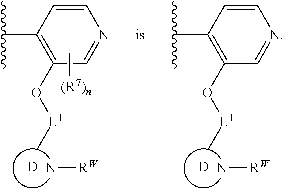

In certain embodiments of Formula (I-a), (I-b), (I-c), (I-d), (I-e), (I-f), (I-g), (I-h), (I-i), (I-j), or (I-k), R^{3a} and R^{3b}, together with the Ring B ring atom to which each is attached, form a fused saturated ring of 4-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-8 ring atoms is optionally substituted with from 1-4 substituents independently selected from the group consisting of oxo, R^{c}, and R^{W}.

In certain embodiments of Formula (I-a), (I-b), (I-c), (I-d), (I-e), (I-f), (I-g), (I-h), (I-i), (I-j), or (I-k) R^{3a} and R^{3b}, together with the Ring B ring atom to which each is attached, form:

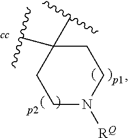

which is optionally substituted with from 1-2 substituents independently selected from the group consisting of oxo and R^{c}, wherein:
p1 and p2 are independently 0, 1, or 2;
R^{Q} is H, R^{d}, C(═O)—W, or S(O)₂W; and
cc represents the point of attachment to C(R^{2a}R^{2b}).

In certain of these embodiments, R^{Q} is H. In certain embodiments, R^{Q} is R^{d}. In certain embodiments, R^{Q} is C₁₋₆ alkyl optionally substituted with from 1-3 independently selected R^{a}. In certain embodiments, R^{Q} is C(═O)—W or S(O)₂W. In certain of these embodiments, W is C₂₋₄ alkenyl. For example, R^{Q} can be C(═O)—CH₂═CH₂.

In certain embodiments of Formula (I-a), (I-b), (I-c), (I-d), (I-e), (I-f), (I-g), (I-h), (I-i), (I-j), or (I-k), R^{3a} and R^{3b}, together with the Ring B ring atom to which each is attached, form

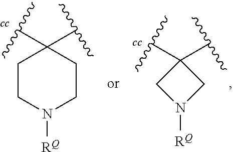

wherein R^{Q} is H, R^{d}, C(═O)—W, or S(O)₂W; and cc represents the point of attachment to C(R^{2a}R^{2b}). In certain of these embodiments, R^{Q} is H. In certain embodiments, R^{Q} is R^{d}. In certain embodiments, R^{Q} is C₁₋₆ alkyl optionally substituted with from 1-3 independently selected R^{a}. In certain embodiments, R^{Q} is C(═O)—W or S(O)₂W. In certain of these embodiments, W is C₂₋₄ alkenyl. For example, R^{Q} can be C(═O)—CH₂═CH₂.

In certain embodiments of Formula (I-a), (I-b), (I-c), (I-d), (I-e), (I-f), (I-g), (I-h), (I-i), (I-j), or (I-k), R^{3a} and R^{3b}, together with the Ring B ring atom to which each is attached, form a fused ring selected from the group consisting of:

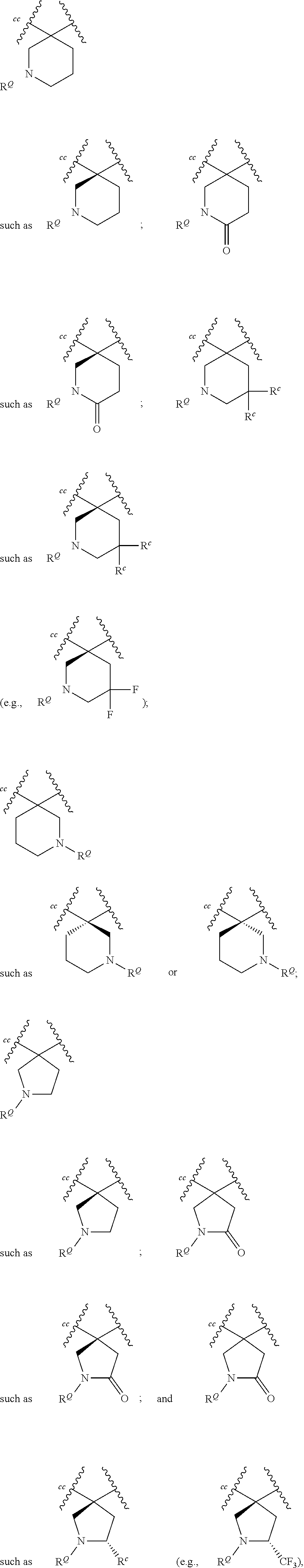

wherein R^{Q} is H, R^{d}, C(═O)—W, or S(O)₂W; and cc represents the point of attachment to C(R^{2a}R^{2b}). In certain of these embodiments, R^{Q} is H. In certain embodiments, R^{Q} is R^{d}. In certain embodiments, R^{Q} is C₁₋₆ alkyl optionally substituted with from 1-3 independently selected R^{a}. In certain embodiments, R^{Q} is C(═O)—W or S(O)₂W. In certain of these embodiments, W is C₂₋₄ alkenyl. For example, R^{Q} can be C(═O)—CH₂═CH₂.

In certain embodiments of Formula (I-a), (I-b), (I-c), (I-d), (I-e), (I-f), (I-g), (I-h), (I-i), (I-j), or (I-k), R^{1c} is H.

In certain embodiments of Formula (I-a), (I-b), (I-c), (I-d), (I-e), (I-f), (I-g), (I-h), (I-i), (I-j), or (I-k), R^{2a} and R^{2b} are H.

In certain embodiments of Formula (I-a), (I-b), (I-c), (I-d), (I-e), (I-f), (I-g), (I-h), (I-i), (I-j), or (I-k), R^{3a} and R^{3A} are H.

In certain embodiments of Formula (I-a), (I-b), (I-c), (I-d), (I-e), (I-f), (I-g), (I-h), (I-i), (I-j), or (I-k), Ring A is

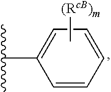

wherein each R^{cB} is an independently selected R^{c}; and m is 1, 2, or 3. In certain of these embodiments, m is 1 or 2, such as 2.

In certain embodiments of Formula (I-a), (I-b), (I-c), (I-d), (I-e), (I-f), (I-g), (I-h), (I-i), (I-j), or (I-k), Ring A is

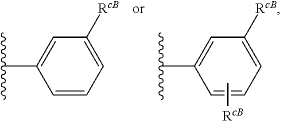

wherein each R^{cB} is independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

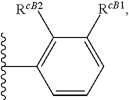

wherein each R^{cB1} is R^{c}; and R^{cB2} is H or R^{c}. In certain of these embodiments, R^{cB1} is halo, such as —F or —Cl, such as —F.

In certain embodiments, R^{cB2} is C₁₋₄ alkoxy or C₁₋₄ haloalkoxy, such as C₁₋₄ alkoxy, such as methoxy.

In certain embodiments of Formula (I-a), (I-b), (I-c), (I-d), (I-e), (I-f), (I-g), (I-h), (I-i), (I-j), or (I-k), Ring A is

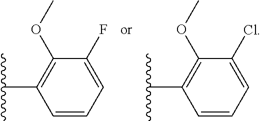

In certain embodiments of Formula (I-a), (I-b), (I-c), (I-d), (I-e), (I-f), (I-g), (I-h), (I-i), (I-j), or (I-k), Ring A is bicyclic heteroaryl including from 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

As non-limiting examples of the foregoing embodiments, Ring A can be quinolinyl, indazolyl, pyrazolopyridyl, or isothiazolopyridyl, each of which is optionally substituted with from 1-2 R^{c}, wherein a ring nitrogen is optionally substituted with R^{d}. For example, Ring A can be:

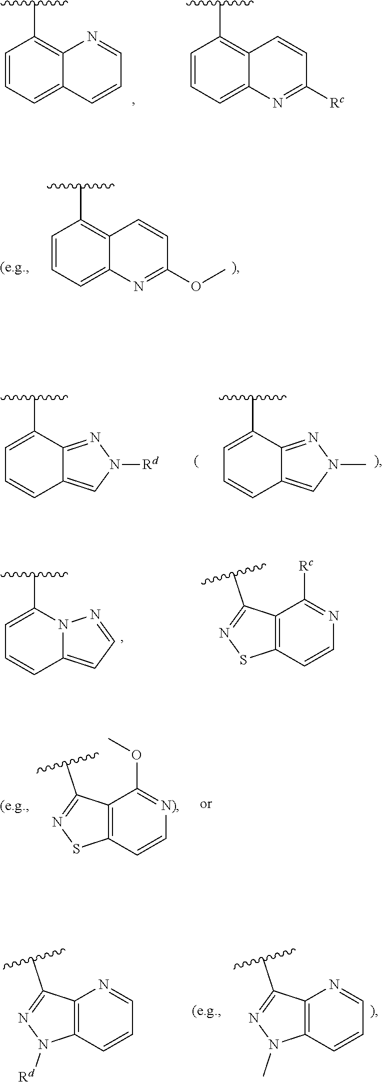

each of which is further optionally substituted with R^{c}.

In certain embodiments of Formula (I-a), (I-b), (I-c), (I-d), (I-e), (I-f), (I-g), (I-h), (I-i), (I-j), or (I-k), n is 0.

In certain embodiments of Formula (I-a), (I-b), (I-c), (I-d), (I-e), (I-f), (I-g), (I-h), (I-i), (I-j), or (I-k), R⁴ is H.

### Compound Provisions

In some embodiments, the compound is other than a compound selected from the group consisting of the structures represented below:

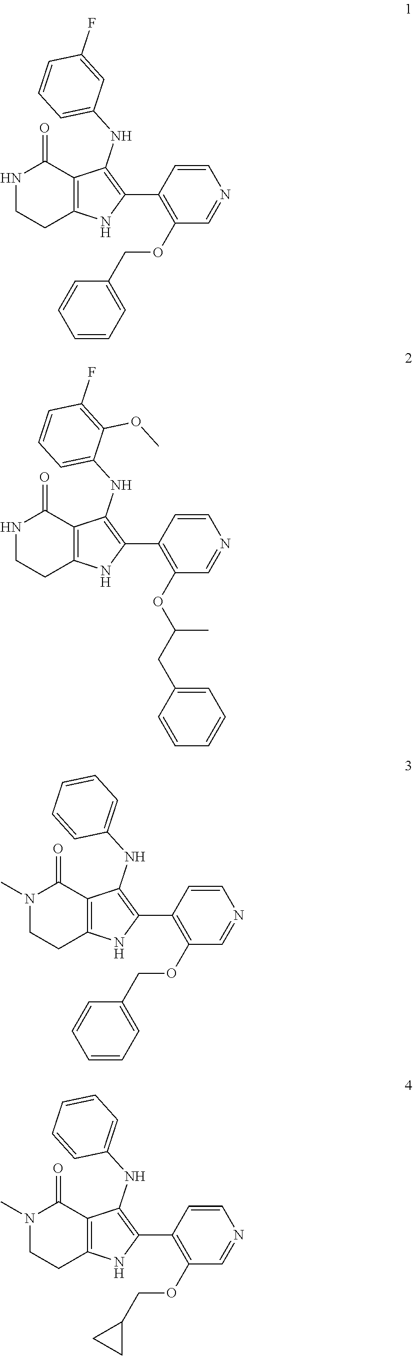

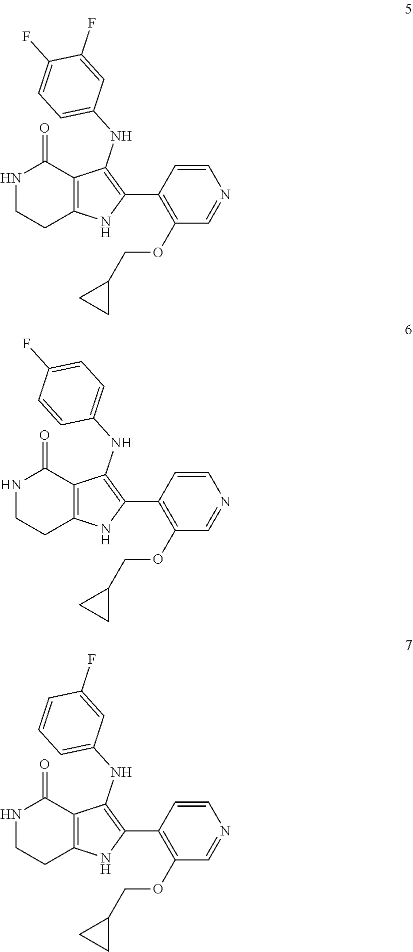

In some embodiments, the compound is other than one or more compounds disclosed in WO 2019/081486, WO 2016/120196, or U.S. Pat. No. 10,428,063, each of which is incorporated herein by reference in its entirety.

In some embodiments, it is provided that when R^{2a}, R^{2b}, R^{3a}, and R^{3A} are each H; R″ is H or methyl; Ring A is phenyl optionally substituted with from 1-2 F; X¹ is —O-L¹-R⁵; and -L¹ is CH₂, then:

R⁵ is other than unsubstituted phenyl or unsubstituted cyclopropyl; and

further provided that the compound is other than: 3-((3-fluoro-2-methoxyphenyl)amino)-2-(3-((1-phenylpropan-2-yl)oxy)pyridin-4-yl)-1,5,6,7-tetrahydro-4H-pyrrolo[3,2-c]pyridin-4-one.

In some embodiments, R⁵ is other than unsubstituted phenyl. In some embodiments, R⁵ is other than unsubstituted cyclopropyl.

In some embodiments, Ring A is other than phenyl optionally substituted with from 1-2 F.

### Non-Limiting Exemplary Compounds

In certain embodiments, the compound is selected from the group consisting of the compounds in Table C₁, or a pharmaceutically acceptable salt thereof.

Table C1

For certain compounds, the symbol * at a chiral center denotes that this chiral center has been resolved (i.e., is a single epimer) and the absolute stereochemistry at that center has not been determined.

| Compound # | Structure |
|---|---|
| 101 | 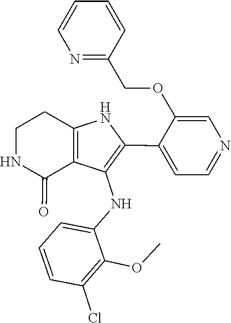 [SMILES: COc1c(Cl)cccc1Nc1c(-c2ccncc2OCc2ccccn2)[nH]c2c1C(=O)NCC2] |
| 102 | 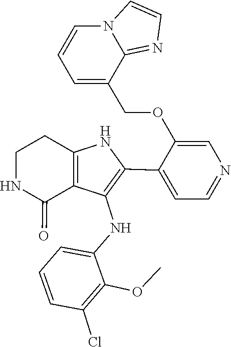 [SMILES: COc1c(Cl)cccc1Nc1c(-c2ccncc2OCc2cccn3ccnc23)[nH]c2c1C(=O)NCC2] |
| 103 | 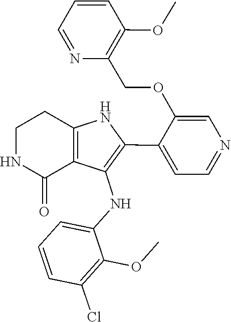 [SMILES: COc1cccnc1COc1cnccc1-c1[nH]c2c(c1Nc1cccc(Cl)c1OC)C(=O)NCC2] |
| 104 | 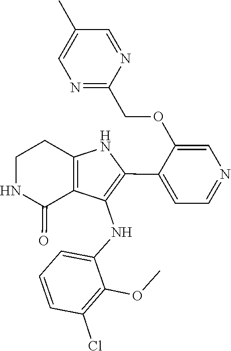 [SMILES: COc1c(Cl)cccc1Nc1c(-c2ccncc2OCc2ncc(C)cn2)[nH]c2c1C(=O)NCC2] |
| 105 | 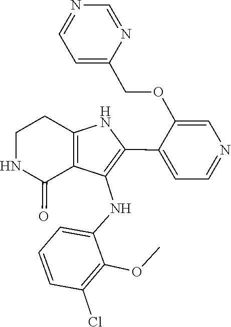 [SMILES: COc1c(Cl)cccc1Nc1c(-c2ccncc2OCc2ccncn2)[nH]c2c1C(=O)NCC2] |
| 106a | 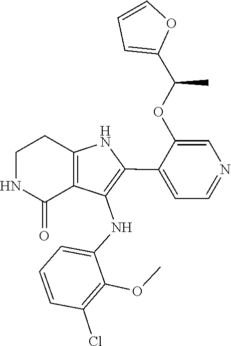 [SMILES: COc1c(Cl)cccc1Nc1c(-c2ccncc2O[C@H](C)c2ccco2)[nH]c2c1C(=O)NCC2] |
| 106b | 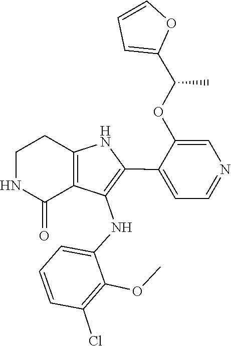 [SMILES: COc1c(Cl)cccc1Nc1c(-c2ccncc2O[C@@H](C)c2ccco2)[nH]c2c1C(=O)NCC2] |
| 107 | 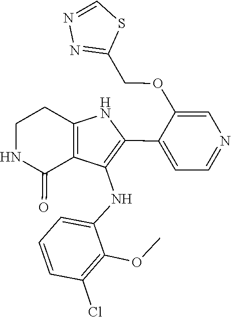 [SMILES: COc1c(Cl)cccc1Nc1c(-c2ccncc2OCc2nncs2)[nH]c2c1C(=O)NCC2] |
| 108 | 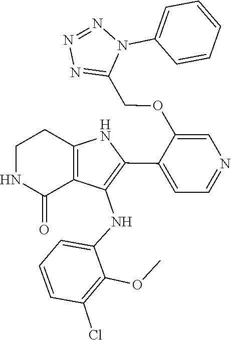 [SMILES: COc1c(Cl)cccc1Nc1c(-c2ccncc2OCc2nnnn2-c2ccccc2)[nH]c2c1C(=O)NCC2] |
| 109 | 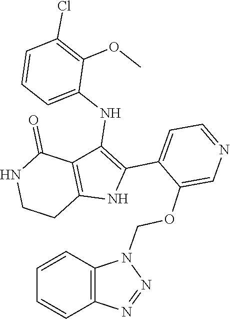 [SMILES: COc1c(Cl)cccc1Nc1c(-c2ccncc2OCn2nnc3ccccc32)[nH]c2c1C(=O)NCC2] |
| 110 | 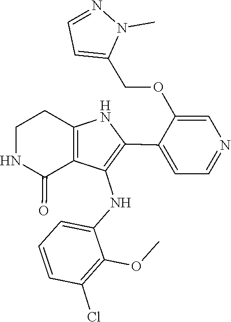 [SMILES: COc1c(Cl)cccc1Nc1c(-c2ccncc2OCc2ccnn2C)[nH]c2c1C(=O)NCC2] |
| 111 | 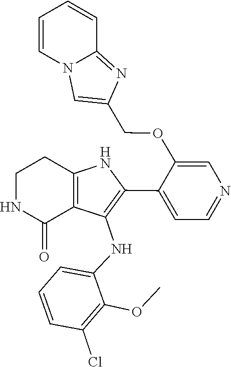 [SMILES: COc1c(Cl)cccc1Nc1c(-c2ccncc2OCc2cn3ccccc3n2)[nH]c2c1C(=O)NCC2] |
| 112 | 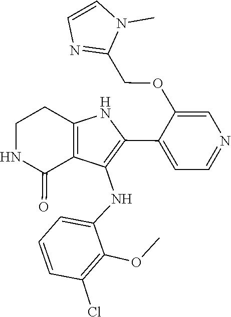 [SMILES: COc1c(Cl)cccc1Nc1c(-c2ccncc2OCc2nccn2C)[nH]c2c1C(=O)NCC2] |
| 113 | 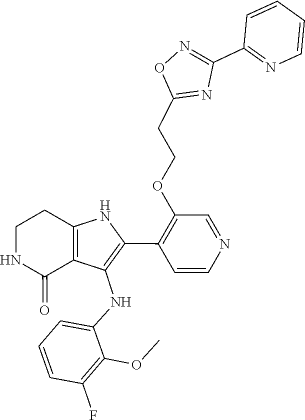 [SMILES: COc1c(F)cccc1Nc1c(-c2ccncc2OCCc2nc(-c3ccccn3)no2)[nH]c2c1C(=O)NCC2] |
| 114 | 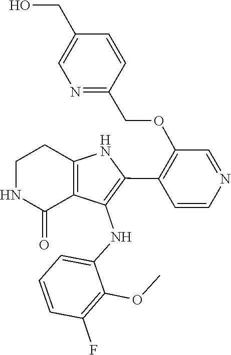 [SMILES: COc1c(F)cccc1Nc1c(-c2ccncc2OCc2ccc(CO)cn2)[nH]c2c1C(=O)NCC2] |
| 115 | 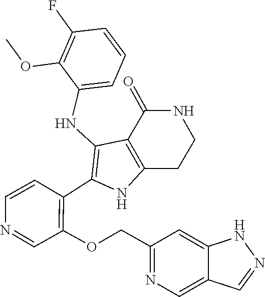 [SMILES: COc1c(F)cccc1Nc1c(-c2ccncc2OCc2cc3[nH]ncc3cn2)[nH]c2c1C(=O)NCC2] |
| 116 | 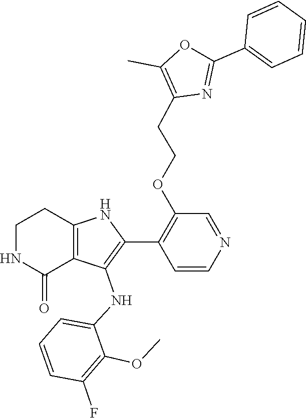 [SMILES: COc1c(F)cccc1Nc1c(-c2ccncc2OCCc2nc(-c3ccccc3)oc2C)[nH]c2c1C(=O)NCC2] |
| 117 | 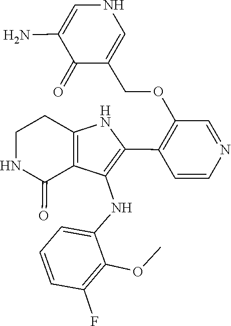 [SMILES: COc1c(F)cccc1Nc1c(-c2ccncc2OCc2c[nH]cc(N)c2=O)[nH]c2c1C(=O)NCC2] |
| 118 | 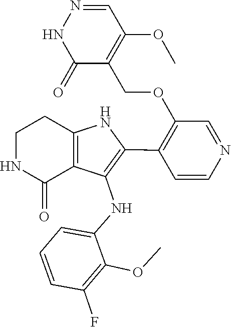 [SMILES: COc1cn[nH]c(=O)c1COc1cnccc1-c1[nH]c2c(c1Nc1cccc(F)c1OC)C(=O)NCC2] |
| 119 | 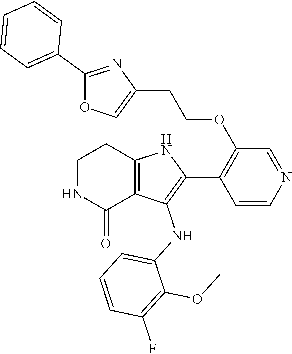 [SMILES: COc1c(F)cccc1Nc1c(-c2ccncc2OCCc2coc(-c3ccccc3)n2)[nH]c2c1C(=O)NCC2] |
| 120 | 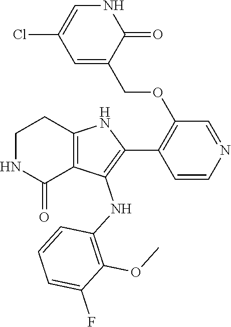 [SMILES: COc1c(F)cccc1Nc1c(-c2ccncc2OCc2cc(Cl)c[nH]c2=O)[nH]c2c1C(=O)NCC2] |
| 121 | 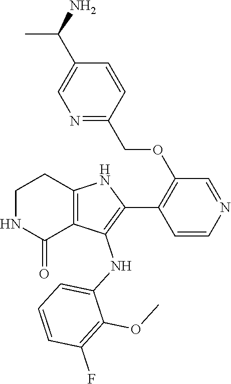 [SMILES: COc1c(F)cccc1Nc1c(-c2ccncc2OCc2ccc([C@@H](C)N)cn2)[nH]c2c1C(=O)NCC2] |
| 122 | 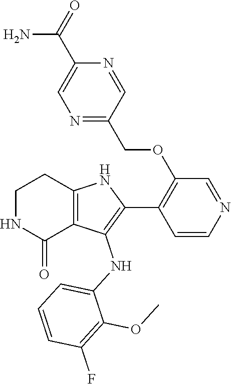 [SMILES: COc1c(F)cccc1Nc1c(-c2ccncc2OCc2cnc(C(N)=O)cn2)[nH]c2c1C(=O)NCC2] |
| 123 | 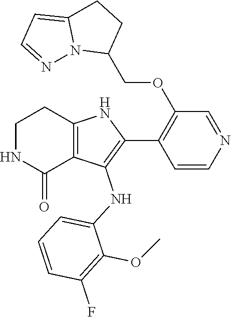 [SMILES: COc1c(F)cccc1Nc1c(-c2ccncc2OCC2CCc3ccnn32)[nH]c2c1C(=O)NCC2] |
| 124 | 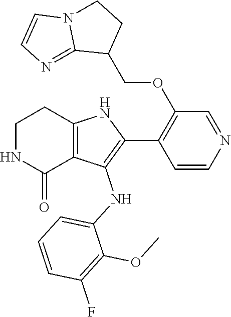 [SMILES: COc1c(F)cccc1Nc1c(-c2ccncc2OCC2CCn3ccnc32)[nH]c2c1C(=O)NCC2] |
| 125 | 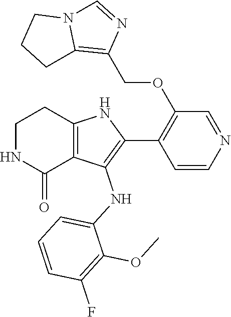 [SMILES: COc1c(F)cccc1Nc1c(-c2ccncc2OCc2ncn3c2CCC3)[nH]c2c1C(=O)NCC2] |
| 126 | 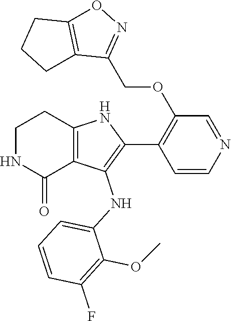 [SMILES: COc1c(F)cccc1Nc1c(-c2ccncc2OCc2noc3c2CCC3)[nH]c2c1C(=O)NCC2] |
| 127 | 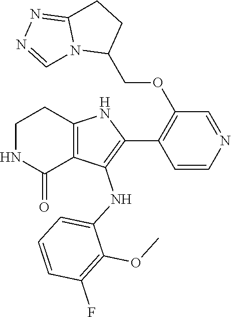 [SMILES: COc1c(F)cccc1Nc1c(-c2ccncc2OCC2CCc3nncn32)[nH]c2c1C(=O)NCC2] |
| 128 | 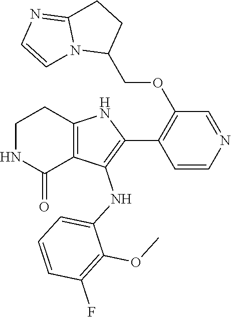 [SMILES: COc1c(F)cccc1Nc1c(-c2ccncc2OCC2CCc3nccn32)[nH]c2c1C(=O)NCC2] |
| 129 | 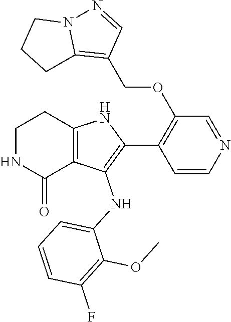 [SMILES: COc1c(F)cccc1Nc1c(-c2ccncc2OCc2cnn3c2CCC3)[nH]c2c1C(=O)NCC2] |
| 130 | 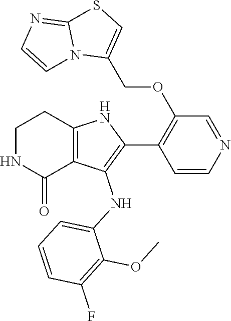 [SMILES: COc1c(F)cccc1Nc1c(-c2ccncc2OCc2csc3nccn23)[nH]c2c1C(=O)NCC2] |
| 131 | 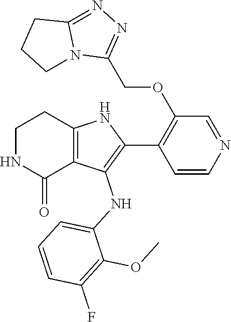 [SMILES: COc1c(F)cccc1Nc1c(-c2ccncc2OCc2nnc3n2CCC3)[nH]c2c1C(=O)NCC2] |
| 132 | 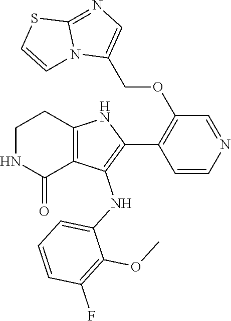 [SMILES: COc1c(F)cccc1Nc1c(-c2ccncc2OCc2cnc3sccn23)[nH]c2c1C(=O)NCC2] |
| 133 | 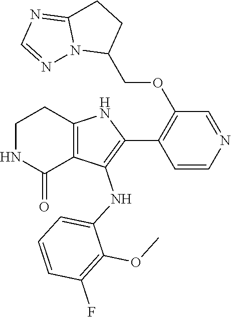 [SMILES: COc1c(F)cccc1Nc1c(-c2ccncc2OCC2CCc3ncnn32)[nH]c2c1C(=O)NCC2] |
| 133a | 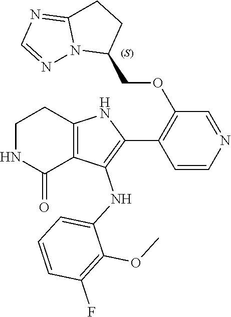 |
| 134 | 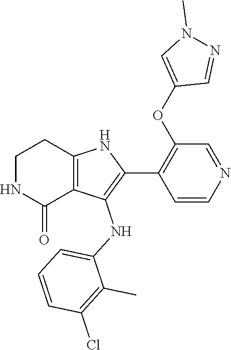 [SMILES: Cc1c(Cl)cccc1Nc1c(-c2ccncc2Oc2cnn(C)c2)[nH]c2c1C(=O)NCC2] |
| 135 | 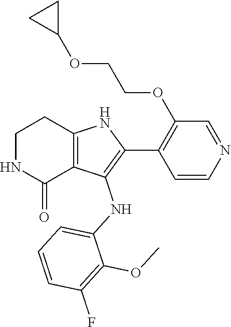 [SMILES: COc1c(F)cccc1Nc1c(-c2ccncc2OCCOC2CC2)[nH]c2c1C(=O)NCC2] |
| 136a | 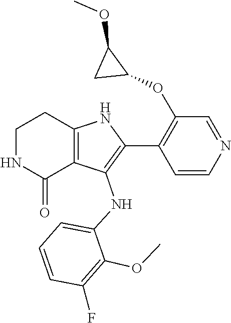 [SMILES: COc1c(F)cccc1Nc1c(-c2ccncc2O[C@@H]2C[C@H]2OC)[nH]c2c1C(=O)NCC2] |
| 136b | 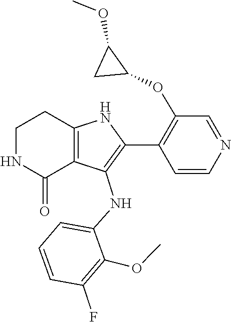 [SMILES: COc1c(F)cccc1Nc1c(-c2ccncc2O[C@@H]2C[C@@H]2OC)[nH]c2c1C(=O)NCC2] |
| 137 | 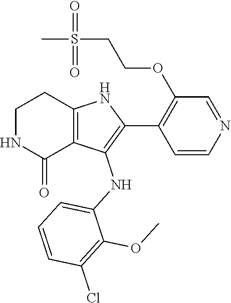 [SMILES: COc1c(Cl)cccc1Nc1c(-c2ccncc2OCCS(C)(=O)=O)[nH]c2c1C(=O)NCC2] |
| 138a | 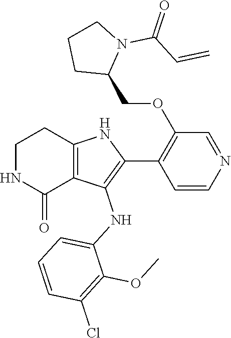 [SMILES: C=CC(=O)N1CCC[C@@H]1COc1cnccc1-c1[nH]c2c(c1Nc1cccc(Cl)c1OC)C(=O)NCC2] |
| 138b | 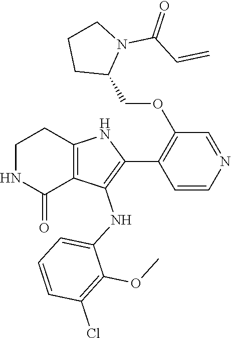 [SMILES: C=CC(=O)N1CCC[C@H]1COc1cnccc1-c1[nH]c2c(c1Nc1cccc(Cl)c1OC)C(=O)NCC2] |
| 139 | 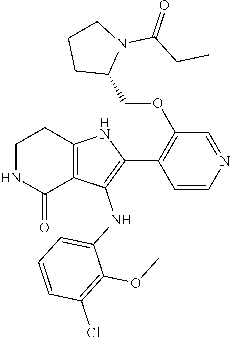 [SMILES: CCC(=O)N1CCC[C@H]1COc1cnccc1-c1[nH]c2c(c1Nc1cccc(Cl)c1OC)C(=O)NCC2] |
| 140 | 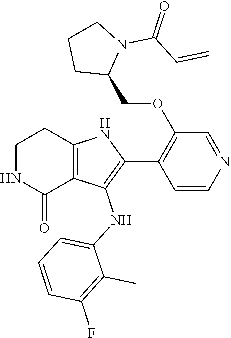 [SMILES: C=CC(=O)N1CCC[C@@H]1COc1cnccc1-c1[nH]c2c(c1Nc1cccc(F)c1C)C(=O)NCC2] |
| 141a | 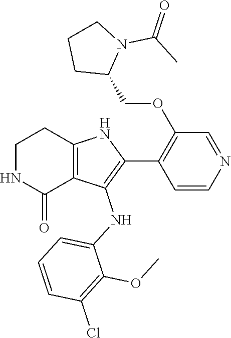 [SMILES: COc1c(Cl)cccc1Nc1c(-c2ccncc2OC[C@@H]2CCCN2C(C)=O)[nH]c2c1C(=O)NCC2] |
| 141b | 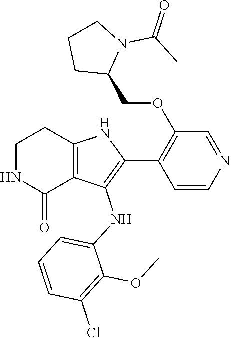 [SMILES: COc1c(Cl)cccc1Nc1c(-c2ccncc2OC[C@H]2CCCN2C(C)=O)[nH]c2c1C(=O)NCC2] |
| 142a | 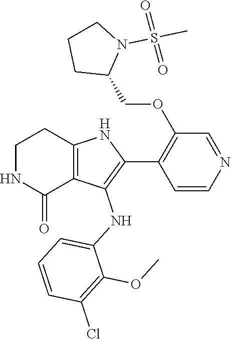 [SMILES: COc1c(Cl)cccc1Nc1c(-c2ccncc2OC[C@@H]2CCCN2S(C)(=O)=O)[nH]c2c1C(=O)NCC2] |
| 142b | 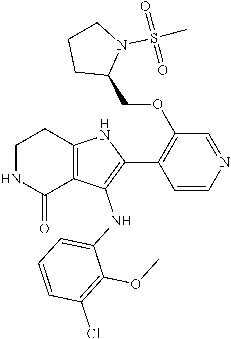 [SMILES: COc1c(Cl)cccc1Nc1c(-c2ccncc2OC[C@H]2CCCN2S(C)(=O)=O)[nH]c2c1C(=O)NCC2] |
| 143 | 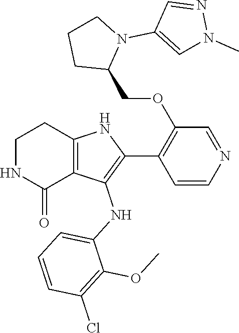 [SMILES: COc1c(Cl)cccc1Nc1c(-c2ccncc2OC[C@H]2CCCN2c2cnn(C)c2)[nH]c2c1C(=O)NCC2] |
| 144a | 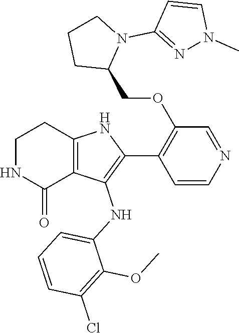 [SMILES: COc1c(Cl)cccc1Nc1c(-c2ccncc2OC[C@H]2CCCN2c2ccn(C)n2)[nH]c2c1C(=O)NCC2] |
| 144b | 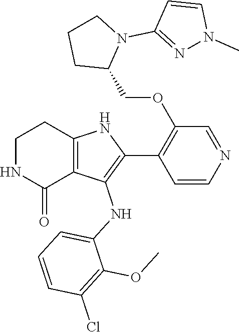 [SMILES: COc1c(Cl)cccc1Nc1c(-c2ccncc2OC[C@@H]2CCCN2c2ccn(C)n2)[nH]c2c1C(=O)NCC2] |
| 145a | 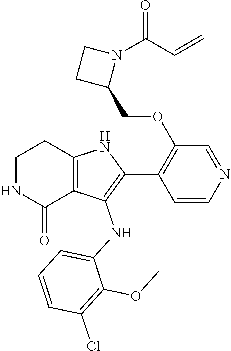 [SMILES: C=CC(=O)N1CC[C@@H]1COc1cnccc1-c1[nH]c2c(c1Nc1cccc(Cl)c1OC)C(=O)NCC2] |
| 145b | 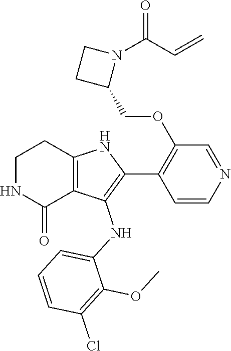 [SMILES: C=CC(=O)N1CC[C@H]1COc1cnccc1-c1[nH]c2c(c1Nc1cccc(Cl)c1OC)C(=O)NCC2] |
| 146 | 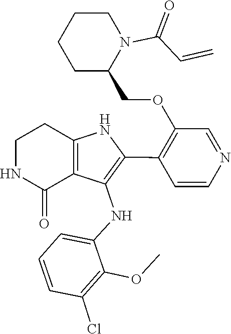 [SMILES: C=CC(=O)N1CCCC[C@@H]1COc1cnccc1-c1[nH]c2c(c1Nc1cccc(Cl)c1OC)C(=O)NCC2] |
| 147a | 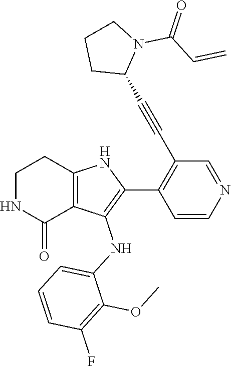 [SMILES: C=CC(=O)N1CCC[C@H]1C#Cc1cnccc1-c1[nH]c2c(c1Nc1cccc(F)c1OC)C(=O)NCC2] |
| 147b | 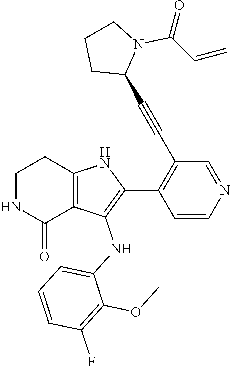 [SMILES: C=CC(=O)N1CCC[C@@H]1C#Cc1cnccc1-c1[nH]c2c(c1Nc1cccc(F)c1OC)C(=O)NCC2] |
| 148a | 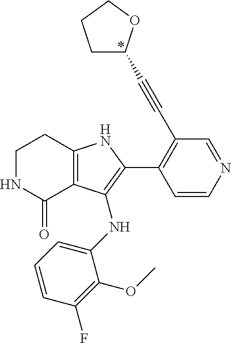 [SMILES: COc1c(F)cccc1Nc1c(-c2ccncc2C#C[C@@H]2CCCO2)[nH]c2c1C(=O)NCC2] |
| 148b | 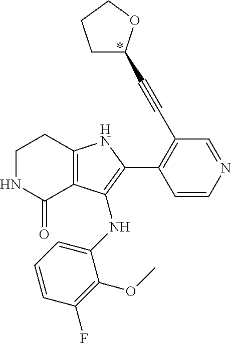 [SMILES: COc1c(F)cccc1Nc1c(-c2ccncc2C#C[C@H]2CCCO2)[nH]c2c1C(=O)NCC2] |
| 149 | 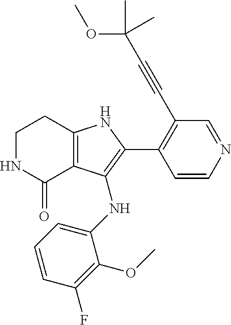 [SMILES: COc1c(F)cccc1Nc1c(-c2ccncc2C#CC(C)(C)OC)[nH]c2c1C(=O)NCC2] |
| 150 | 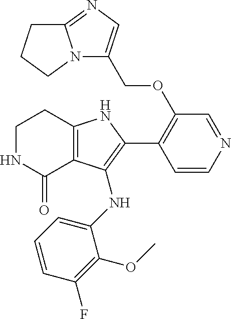 [SMILES: COc1c(F)cccc1Nc1c(-c2ccncc2OCc2cnc3n2CCC3)[nH]c2c1C(=O)NCC2] |
| 151 | 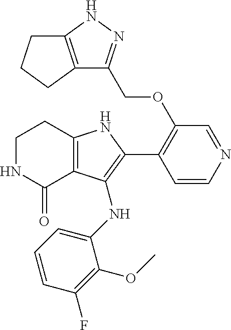 [SMILES: COc1c(F)cccc1Nc1c(-c2ccncc2OCc2n[nH]c3c2CCC3)[nH]c2c1C(=O)NCC2] |
| 152 | 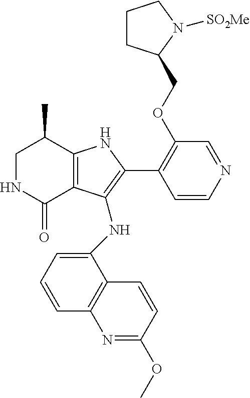 [SMILES: COc1ccc2c(Nc3c(-c4ccncc4OC[C@H]4CCCN4S(C)(=O)=O)[nH]c4c3C(=O)NC[C@H]4C)cccc2n1] |
| 153 | 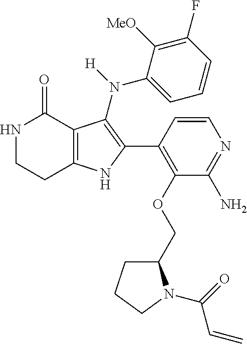 [SMILES: C=CC(=O)N1CCC[C@H]1COc1c(-c2[nH]c3c(c2Nc2cccc(F)c2OC)C(=O)NCC3)ccnc1N] |
| 154 | 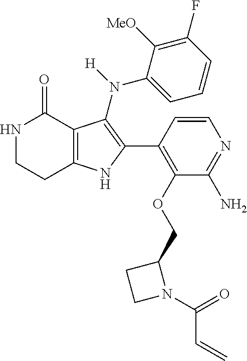 [SMILES: C=CC(=O)N1CC[C@H]1COc1c(-c2[nH]c3c(c2Nc2cccc(F)c2OC)C(=O)NCC3)ccnc1N] |
| 155 | 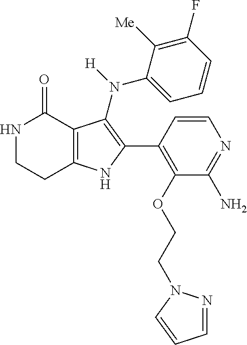 [SMILES: Cc1c(F)cccc1Nc1c(-c2ccnc(N)c2OCCn2cccn2)[nH]c2c1C(=O)NCC2] |
| 156 | 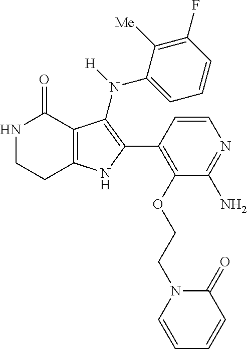 [SMILES: Cc1c(F)cccc1Nc1c(-c2ccnc(N)c2OCCn2ccccc2=O)[nH]c2c1C(=O)NCC2] |
| 157 | 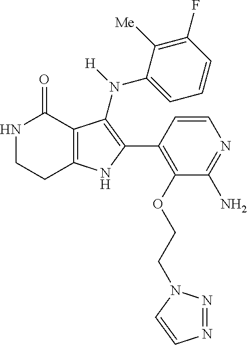 [SMILES: Cc1c(F)cccc1Nc1c(-c2ccnc(N)c2OCCn2ccnn2)[nH]c2c1C(=O)NCC2] |
| 158 | 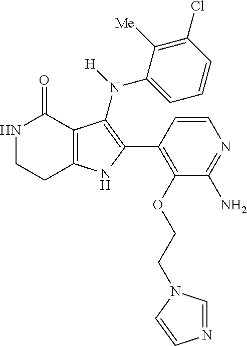 [SMILES: Cc1c(Cl)cccc1Nc1c(-c2ccnc(N)c2OCCn2ccnc2)[nH]c2c1C(=O)NCC2] |
| 159 | 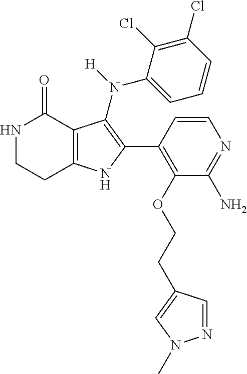 [SMILES: Cn1cc(CCOc2c(-c3[nH]c4c(c3Nc3cccc(Cl)c3Cl)C(=O)NCC4)ccnc2N)cn1] |
| 160 | 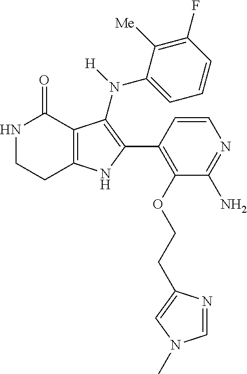 [SMILES: Cc1c(F)cccc1Nc1c(-c2ccnc(N)c2OCCc2cn(C)cn2)[nH]c2c1C(=O)NCC2] |
| 161 | 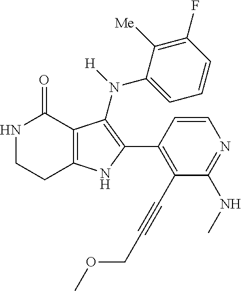 [SMILES: CNc1nccc(-c2[nH]c3c(c2Nc2cccc(F)c2C)C(=O)NCC3)c1C#CCOC] |
| 162 | 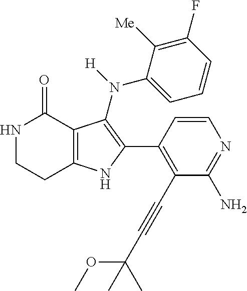 [SMILES: COC(C)(C)C#Cc1c(-c2[nH]c3c(c2Nc2cccc(F)c2C)C(=O)NCC3)ccnc1N] |
| 163 | 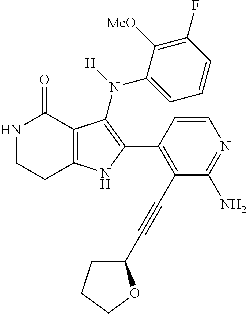 [SMILES: COc1c(F)cccc1Nc1c(-c2ccnc(N)c2C#C[C@@H]2CCCO2)[nH]c2c1C(=O)NCC2] |
| 164 | 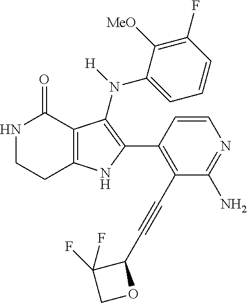 [SMILES: COc1c(F)cccc1Nc1c(-c2ccnc(N)c2C#C[C@H]2OCC2(F)F)[nH]c2c1C(=O)NCC2] |
| 165 | 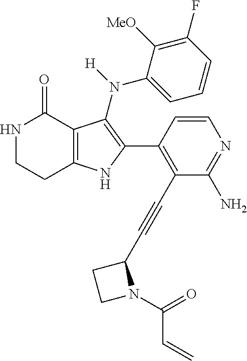 [SMILES: C=CC(=O)N1CC[C@H]1C#Cc1c(-c2[nH]c3c(c2Nc2cccc(F)c2OC)C(=O)NCC3)ccnc1N] |
| 166 | 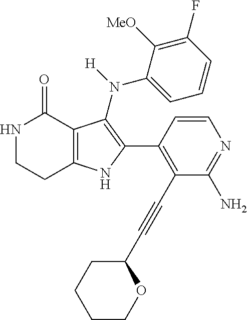 [SMILES: COc1c(F)cccc1Nc1c(-c2ccnc(N)c2C#C[C@@H]2CCCCO2)[nH]c2c1C(=O)NCC2] |
| 167 | 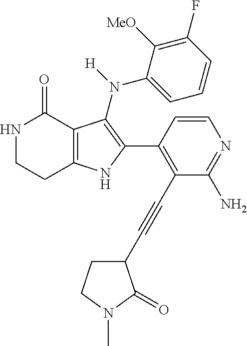 [SMILES: COc1c(F)cccc1Nc1c(-c2ccnc(N)c2C#CC2CCN(C)C2=O)[nH]c2c1C(=O)NCC2] |
| 168 | 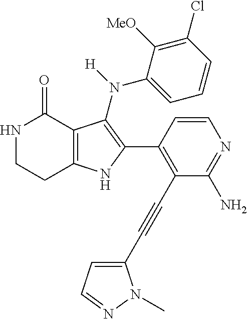 [SMILES: COc1c(Cl)cccc1Nc1c(-c2ccnc(N)c2C#Cc2ccnn2C)[nH]c2c1C(=O)NCC2] |
| 169 | 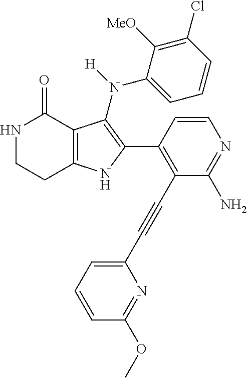 [SMILES: COc1cccc(C#Cc2c(-c3[nH]c4c(c3Nc3cccc(Cl)c3OC)C(=O)NCC4)ccnc2N)n1] |
| 170 | 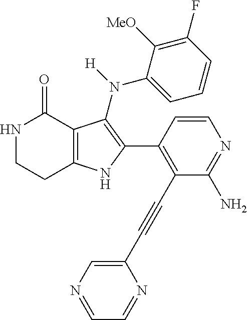 [SMILES: COc1c(F)cccc1Nc1c(-c2ccnc(N)c2C#Cc2cnccn2)[nH]c2c1C(=O)NCC2] |
| 171 | 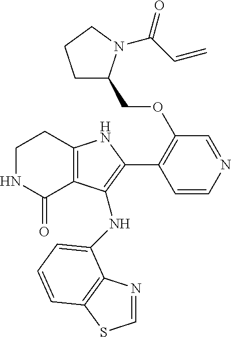 [SMILES: C=CC(=O)N1CCC[C@@H]1COc1cnccc1-c1[nH]c2c(c1Nc1cccc3scnc13)C(=O)NCC2] |
| 172 | 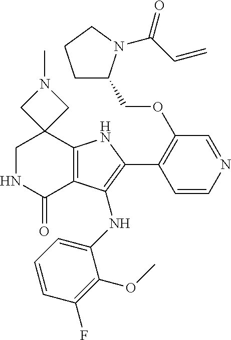 [SMILES: C=CC(=O)N1CCC[C@H]1COc1cnccc1-c1[nH]c2c(c1Nc1cccc(F)c1OC)C(=O)NCC21CN(C)C1] |
| 173 | 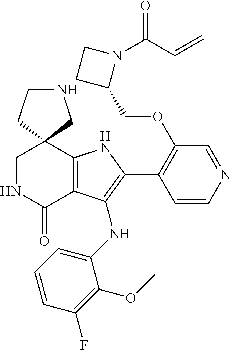 [SMILES: C=CC(=O)N1CC[C@H]1COc1cnccc1-c1[nH]c2c(c1Nc1cccc(F)c1OC)C(=O)NC[C@]21CCNC1] |
| 174 | 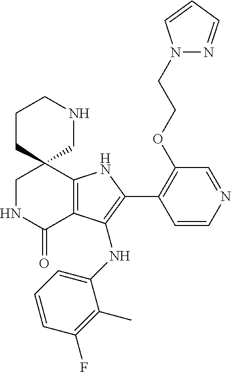 [SMILES: Cc1c(F)cccc1Nc1c(-c2ccncc2OCCn2cccn2)[nH]c2c1C(=O)NC[C@@]21CCCNC1] |
| 175 | 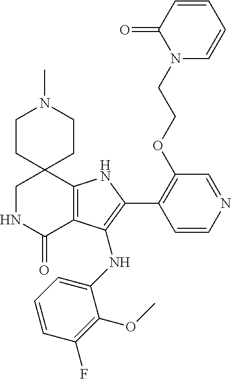 [SMILES: COc1c(F)cccc1Nc1c(-c2ccncc2OCCn2ccccc2=O)[nH]c2c1C(=O)NCC21CCN(C)CC1] |
| 176 | 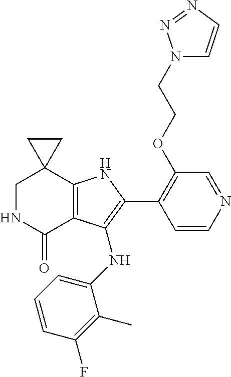 [SMILES: Cc1c(F)cccc1Nc1c(-c2ccncc2OCCn2ccnn2)[nH]c2c1C(=O)NCC21CC1] |
| 177 | 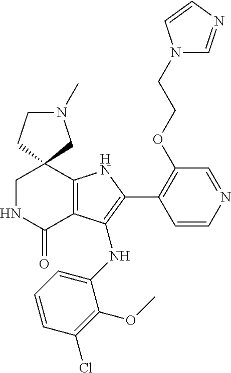 [SMILES: COc1c(Cl)cccc1Nc1c(-c2ccncc2OCCn2ccnc2)[nH]c2c1C(=O)NC[C@]21CCN(C)C1] |
| 178 | 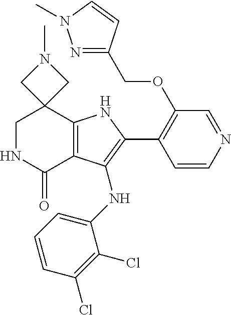 [SMILES: CN1CC2(CNC(=O)c3c2[nH]c(-c2ccncc2OCc2ccn(C)n2)c3Nc2cccc(Cl)c2Cl)C1] |
| 179 | 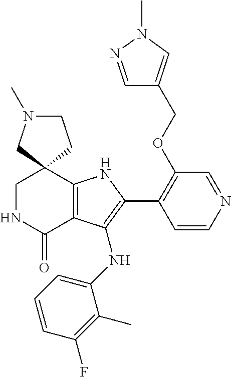 [SMILES: Cc1c(F)cccc1Nc1c(-c2ccncc2OCc2cnn(C)c2)[nH]c2c1C(=O)NC[C@]21CCN(C)C1] |
| 180 | 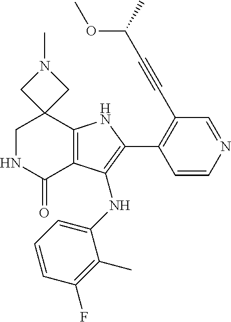 [SMILES: CO[C@H](C)C#Cc1cnccc1-c1[nH]c2c(c1Nc1cccc(F)c1C)C(=O)NCC21CN(C)C1] |
| 181 | 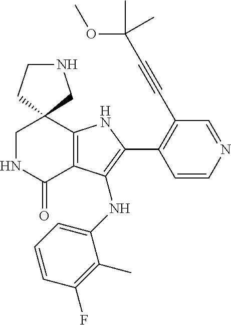 [SMILES: COC(C)(C)C#Cc1cnccc1-c1[nH]c2c(c1Nc1cccc(F)c1C)C(=O)NC[C@]21CCNC1] |
| 182 | 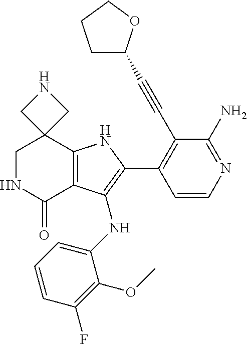 [SMILES: COc1c(F)cccc1Nc1c(-c2ccnc(N)c2C#C[C@@H]2CCCO2)[nH]c2c1C(=O)NCC21CNC1] |
| 183 | 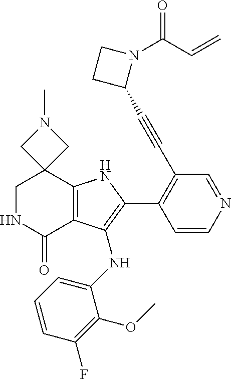 [SMILES: C=CC(=O)N1CC[C@H]1C#Cc1cnccc1-c1[nH]c2c(c1Nc1cccc(F)c1OC)C(=O)NCC21CN(C)C1] |
| 184 | 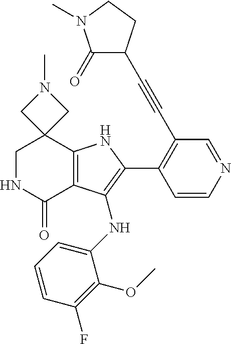 [SMILES: COc1c(F)cccc1Nc1c(-c2ccncc2C#CC2CCN(C)C2=O)[nH]c2c1C(=O)NCC21CN(C)C1] |
| 185 | 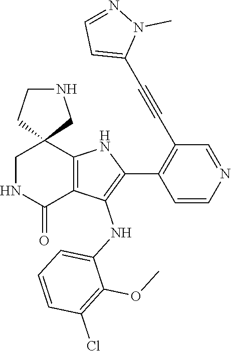 [SMILES: COc1c(Cl)cccc1Nc1c(-c2ccncc2C#Cc2ccnn2C)[nH]c2c1C(=O)NC[C@]21CCNC1] |
| 186 | 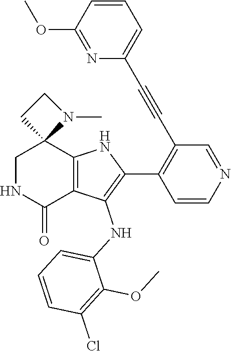 [SMILES: COc1cccc(C#Cc2cnccc2-c2[nH]c3c(c2Nc2cccc(Cl)c2OC)C(=O)NC[C@]32CCN2C)n1] |
| 187 | 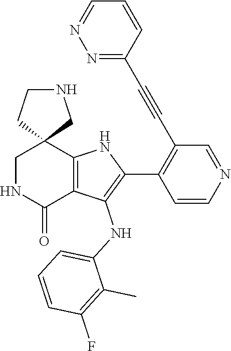 [SMILES: Cc1c(F)cccc1Nc1c(-c2ccncc2C#Cc2cccnn2)[nH]c2c1C(=O)NC[C@]21CCNC1] |
| 188 | 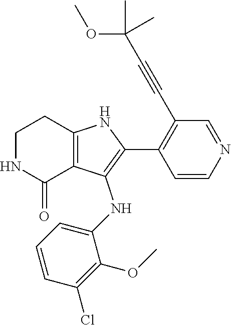 [SMILES: COc1c(Cl)cccc1Nc1c(-c2ccncc2C#CC(C)(C)OC)[nH]c2c1C(=O)NCC2] |
| 189 | 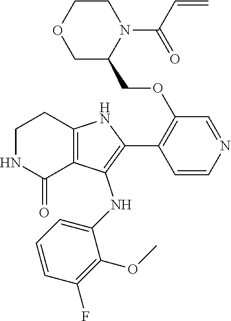 [SMILES: C=CC(=O)N1CCOC[C@@H]1COc1cnccc1-c1[nH]c2c(c1Nc1cccc(F)c1OC)C(=O)NCC2] |
| 190 | 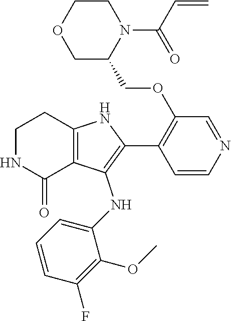 [SMILES: C=CC(=O)N1CCOC[C@H]1COc1cnccc1-c1[nH]c2c(c1Nc1cccc(F)c1OC)C(=O)NCC2] |
| 191 | 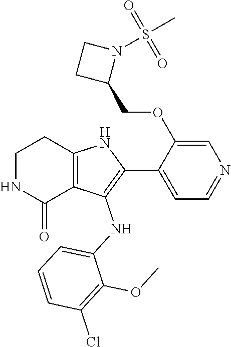 [SMILES: COc1c(Cl)cccc1Nc1c(-c2ccncc2OC[C@H]2CCN2S(C)(=O)=O)[nH]c2c1C(=O)NCC2] |
| 192 | 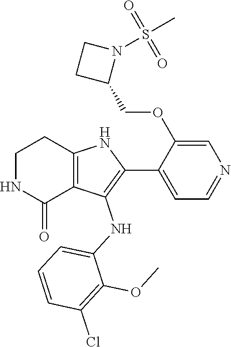 [SMILES: COc1c(Cl)cccc1Nc1c(-c2ccncc2OC[C@@H]2CCN2S(C)(=O)=O)[nH]c2c1C(=O)NCC2] |
| 193 | 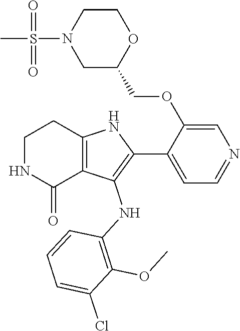 [SMILES: COc1c(Cl)cccc1Nc1c(-c2ccncc2OC[C@@H]2CN(S(C)(=O)=O)CCO2)[nH]c2c1C(=O)NCC2] |
| 194 | 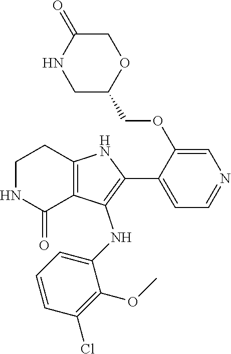 [SMILES: COc1c(Cl)cccc1Nc1c(-c2ccncc2OC[C@@H]2CNC(=O)CO2)[nH]c2c1C(=O)NCC2] |
| 195 | 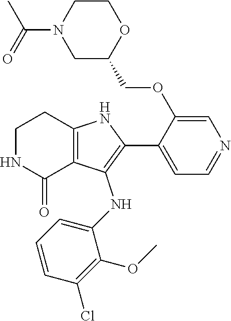 [SMILES: COc1c(Cl)cccc1Nc1c(-c2ccncc2OC[C@@H]2CN(C(C)=O)CCO2)[nH]c2c1C(=O)NCC2] |
| 196 | 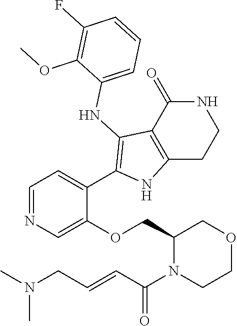 [SMILES: COc1c(F)cccc1Nc1c(-c2ccncc2OC[C@H]2COCCN2C(=O)/C=C/CN(C)C)[nH]c2c1C(=O)NCC2] |
| 197 | 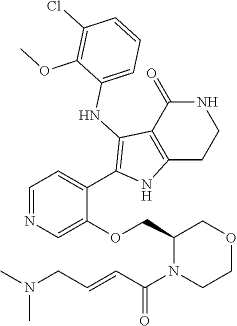 [SMILES: COc1c(Cl)cccc1Nc1c(-c2ccncc2OC[C@H]2COCCN2C(=O)/C=C/CN(C)C)[nH]c2c1C(=O)NCC2] |
| 198 | 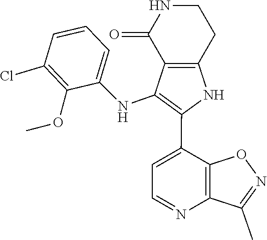 [SMILES: COc1c(Cl)cccc1Nc1c(-c2ccnc3c(C)noc23)[nH]c2c1C(=O)NCC2] |
| 199 | 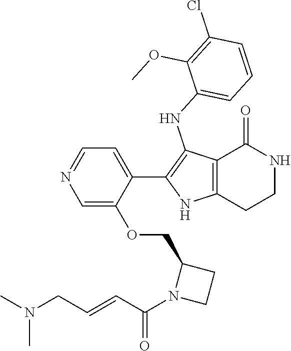 [SMILES: COc1c(Cl)cccc1Nc1c(-c2ccncc2OC[C@H]2CCN2C(=O)/C=C/CN(C)C)[nH]c2c1C(=O)NCC2] |
| 200 | 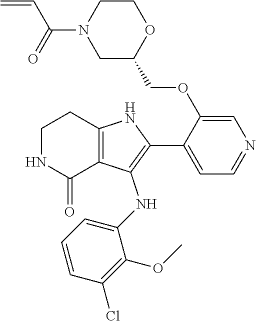 [SMILES: C=CC(=O)N1CCO[C@H](COc2cnccc2-c2[nH]c3c(c2Nc2cccc(Cl)c2OC)C(=O)NCC3)C1] |
| 201 | 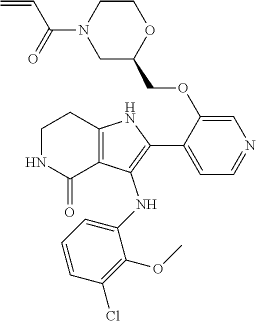 [SMILES: C=CC(=O)N1CCO[C@@H](COc2cnccc2-c2[nH]c3c(c2Nc2cccc(Cl)c2OC)C(=O)NCC3)C1] |
| 202 | 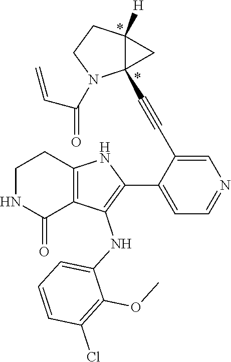 [SMILES: C=CC(=O)N1CC[C@@H]2C[C@@]21C#Cc1cnccc1-c1[nH]c2c(c1Nc1cccc(Cl)c1OC)C(=O)NCC2] |
| 203 | 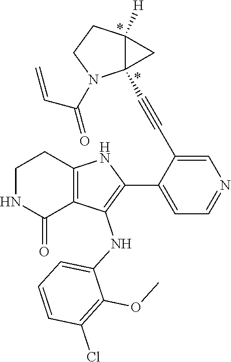 [SMILES: C=CC(=O)N1CC[C@H]2C[C@]21C#Cc1cnccc1-c1[nH]c2c(c1Nc1cccc(Cl)c1OC)C(=O)NCC2] |
| 204 | 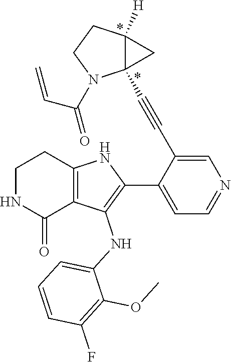 [SMILES: C=CC(=O)N1CC[C@H]2C[C@]21C#Cc1cnccc1-c1[nH]c2c(c1Nc1cccc(F)c1OC)C(=O)NCC2] |
| 205 | 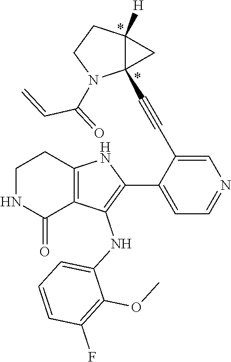 [SMILES: C=CC(=O)N1CC[C@@H]2C[C@@]21C#Cc1cnccc1-c1[nH]c2c(c1Nc1cccc(F)c1OC)C(=O)NCC2] |
| 206 | 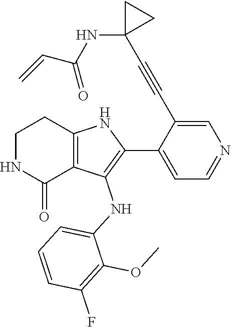 [SMILES: C=CC(=O)NC1(C#Cc2cnccc2-c2[nH]c3c(c2Nc2cccc(F)c2OC)C(=O)NCC3)CC1] |
| 207 | 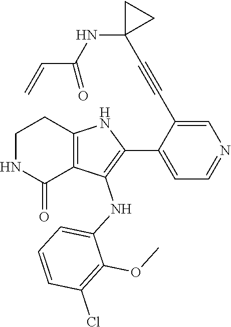 [SMILES: C=CC(=O)NC1(C#Cc2cnccc2-c2[nH]c3c(c2Nc2cccc(Cl)c2OC)C(=O)NCC3)CC1] |
| 208 | 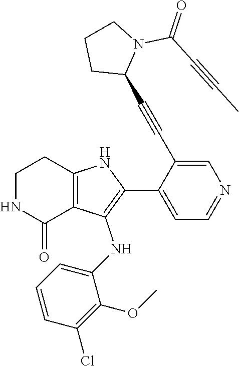 [SMILES: CC#CC(=O)N1CCC[C@@H]1C#Cc1cnccc1-c1[nH]c2c(c1Nc1cccc(Cl)c1OC)C(=O)NCC2] |
| 209 | 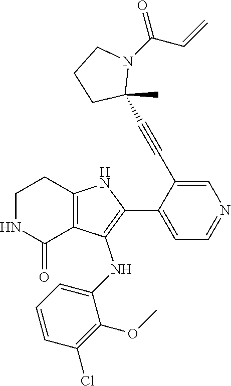 [SMILES: C=CC(=O)N1CCC[C@@]1(C)C#Cc1cnccc1-c1[nH]c2c(c1Nc1cccc(Cl)c1OC)C(=O)NCC2] |
| 210 | 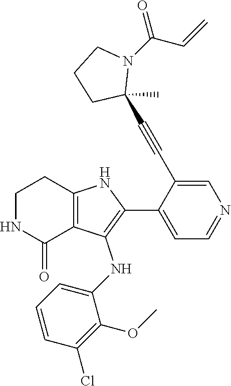 [SMILES: C=CC(=O)N1CCC[C@]1(C)C#Cc1cnccc1-c1[nH]c2c(c1Nc1cccc(Cl)c1OC)C(=O)NCC2] |
| 211 | 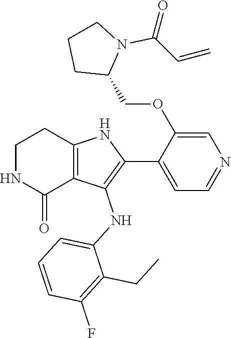 [SMILES: C=CC(=O)N1CCC[C@H]1COc1cnccc1-c1[nH]c2c(c1Nc1cccc(F)c1CC)C(=O)NCC2] |
| 212 | 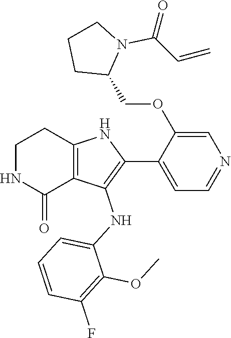 [SMILES: C=CC(=O)N1CCC[C@H]1COc1cnccc1-c1[nH]c2c(c1Nc1cccc(F)c1OC)C(=O)NCC2] |
| 213 | 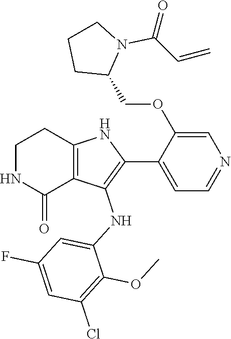 [SMILES: C=CC(=O)N1CCC[C@H]1COc1cnccc1-c1[nH]c2c(c1Nc1cc(F)cc(Cl)c1OC)C(=O)NCC2] |
| 214 | 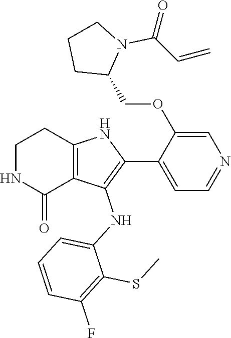 [SMILES: C=CC(=O)N1CCC[C@H]1COc1cnccc1-c1[nH]c2c(c1Nc1cccc(F)c1SC)C(=O)NCC2] |
| 215 | 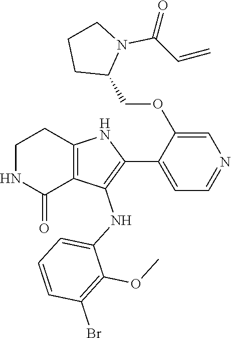 [SMILES: C=CC(=O)N1CCC[C@H]1COc1cnccc1-c1[nH]c2c(c1Nc1cccc(Br)c1OC)C(=O)NCC2] |
| 216 | 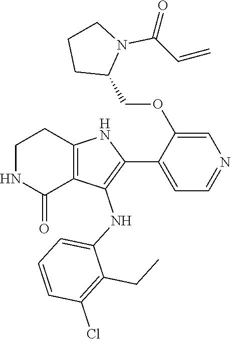 [SMILES: C=CC(=O)N1CCC[C@H]1COc1cnccc1-c1[nH]c2c(c1Nc1cccc(Cl)c1CC)C(=O)NCC2] |
| 217 | 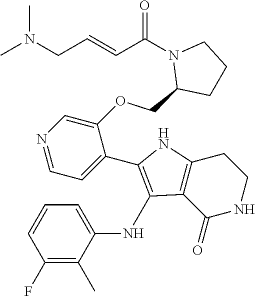 [SMILES: Cc1c(F)cccc1Nc1c(-c2ccncc2OC[C@@H]2CCCN2C(=O)/C=C/CN(C)C)[nH]c2c1C(=O)NCC2] |
| 218 | 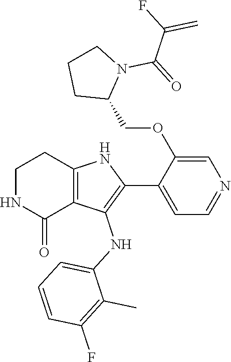 [SMILES: C=C(F)C(=O)N1CCC[C@H]1COc1cnccc1-c1[nH]c2c(c1Nc1cccc(F)c1C)C(=O)NCC2] |
| 219 | 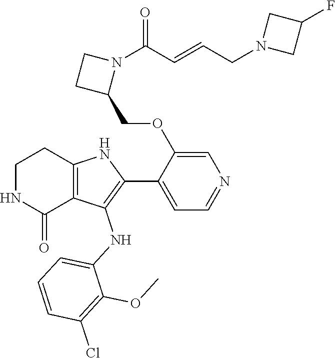 [SMILES: COc1c(Cl)cccc1Nc1c(-c2ccncc2OC[C@H]2CCN2C(=O)/C=C/CN2CC(F)C2)[nH]c2c1C(=O)NCC2] |
| 220 | 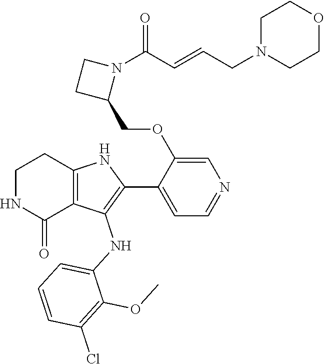 [SMILES: COc1c(Cl)cccc1Nc1c(-c2ccncc2OC[C@H]2CCN2C(=O)/C=C/CN2CCOCC2)[nH]c2c1C(=O)NCC2] |
| 221 | 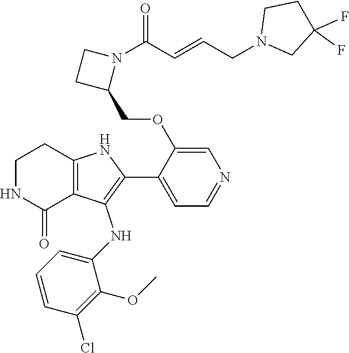 [SMILES: COc1c(Cl)cccc1Nc1c(-c2ccncc2OC[C@H]2CCN2C(=O)/C=C/CN2CCC(F)(F)C2)[nH]c2c1C(=O)NCC2] |
| 222 | 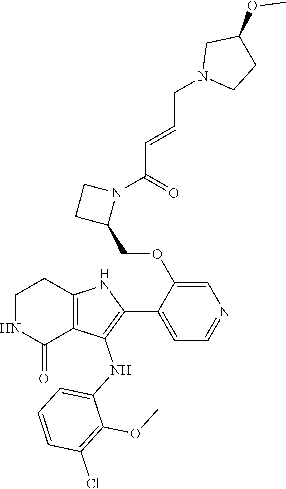 [SMILES: COc1c(Cl)cccc1Nc1c(-c2ccncc2OC[C@H]2CCN2C(=O)/C=C/CN2CC[C@H](OC)C2)[nH]c2c1C(=O)NCC2] |
| 223 | 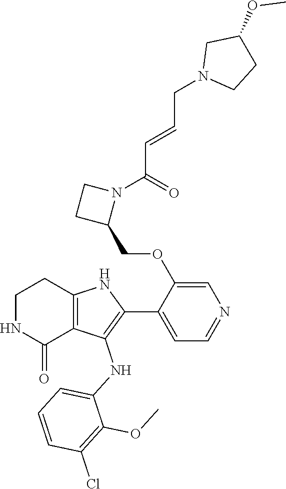 [SMILES: COc1c(Cl)cccc1Nc1c(-c2ccncc2OC[C@H]2CCN2C(=O)/C=C/CN2CC[C@@H](OC)C2)[nH]c2c1C(=O)NCC2] |
| 224 | 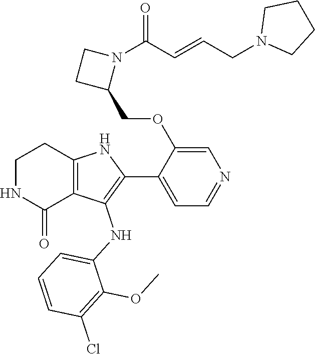 [SMILES: COc1c(Cl)cccc1Nc1c(-c2ccncc2OC[C@H]2CCN2C(=O)/C=C/CN2CCCC2)[nH]c2c1C(=O)NCC2] |
| 225 | 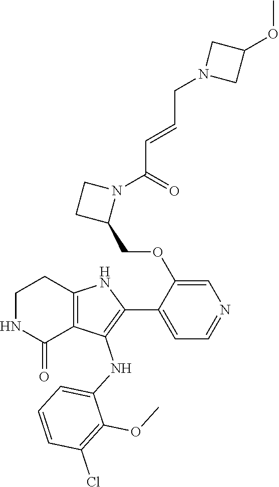 [SMILES: COc1c(Cl)cccc1Nc1c(-c2ccncc2OC[C@H]2CCN2C(=O)/C=C/CN2CC(OC)C2)[nH]c2c1C(=O)NCC2] |
| 226 | 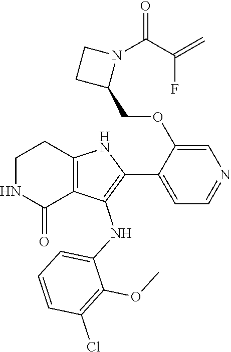 [SMILES: C=C(F)C(=O)N1CC[C@@H]1COc1cnccc1-c1[nH]c2c(c1Nc1cccc(Cl)c1OC)C(=O)NCC2] |
| 227 | 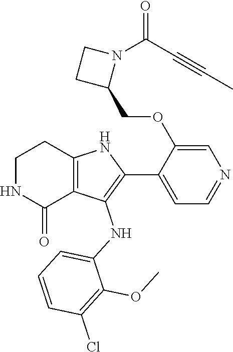 [SMILES: CC#CC(=O)N1CC[C@@H]1COc1cnccc1-c1[nH]c2c(c1Nc1cccc(Cl)c1OC)C(=O)NCC2] |
| 228 | 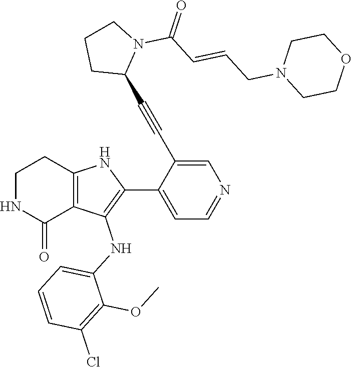 [SMILES: COc1c(Cl)cccc1Nc1c(-c2ccncc2C#C[C@H]2CCCN2C(=O)/C=C/CN2CCOCC2)[nH]c2c1C(=O)NCC2] |
| 229 | 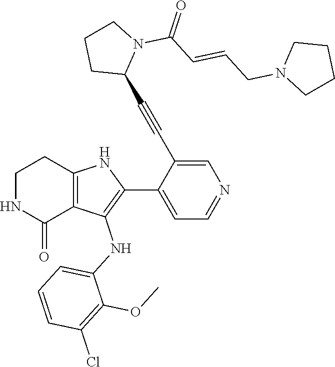 [SMILES: COc1c(Cl)cccc1Nc1c(-c2ccncc2C#C[C@H]2CCCN2C(=O)/C=C/CN2CCCC2)[nH]c2c1C(=O)NCC2] |
| 230 | 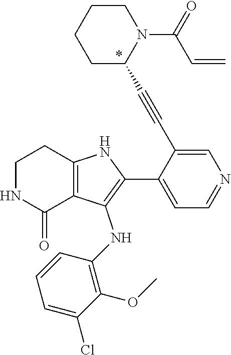 [SMILES: C=CC(=O)N1CCCC[C@H]1C#Cc1cnccc1-c1[nH]c2c(c1Nc1cccc(Cl)c1OC)C(=O)NCC2] |
| 231 | 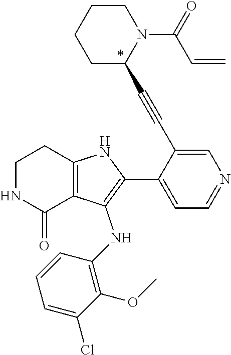 [SMILES: C=CC(=O)N1CCCC[C@@H]1C#Cc1cnccc1-c1[nH]c2c(c1Nc1cccc(Cl)c1OC)C(=O)NCC2] |
| 232 | 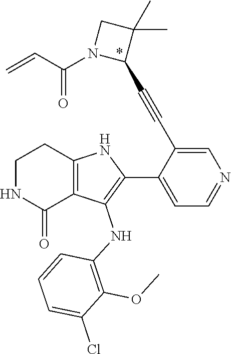 [SMILES: C=CC(=O)N1CC(C)(C)[C@H]1C#Cc1cnccc1-c1[nH]c2c(c1Nc1cccc(Cl)c1OC)C(=O)NCC2] |
| 233 | 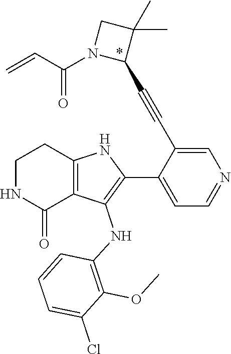 [SMILES: C=CC(=O)N1CC(C)(C)[C@H]1C#Cc1cnccc1-c1[nH]c2c(c1Nc1cccc(Cl)c1OC)C(=O)NCC2] |
| 234 | 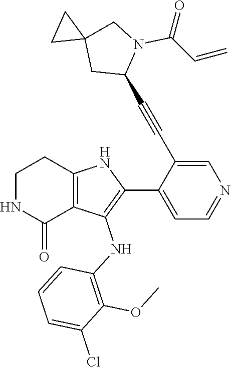 [SMILES: C=CC(=O)N1CC2(CC2)C[C@@H]1C#Cc1cnccc1-c1[nH]c2c(c1Nc1cccc(Cl)c1OC)C(=O)NCC2] |
| 235 | 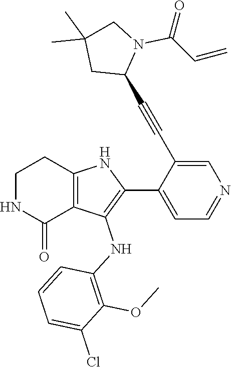 [SMILES: C=CC(=O)N1CC(C)(C)C[C@@H]1C#Cc1cnccc1-c1[nH]c2c(c1Nc1cccc(Cl)c1OC)C(=O)NCC2] |
| 236 | 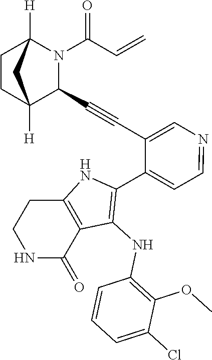 [SMILES: C=CC(=O)N1[C@H]2CC[C@H](C2)[C@@H]1C#Cc1cnccc1-c1[nH]c2c(c1Nc1cccc(Cl)c1OC)C(=O)NCC2] |
| 237 | 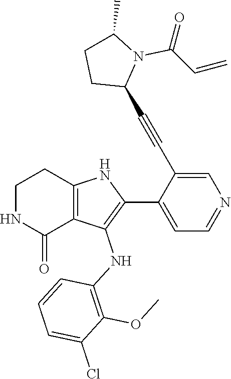 [SMILES: C=CC(=O)N1[C@@H](C)CC[C@@H]1C#Cc1cnccc1-c1[nH]c2c(c1Nc1cccc(Cl)c1OC)C(=O)NCC2] |
| 238 | 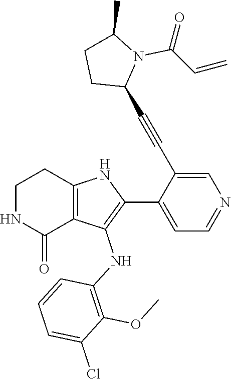 [SMILES: C=CC(=O)N1[C@H](C)CC[C@@H]1C#Cc1cnccc1-c1[nH]c2c(c1Nc1cccc(Cl)c1OC)C(=O)NCC2] |
| 239 | 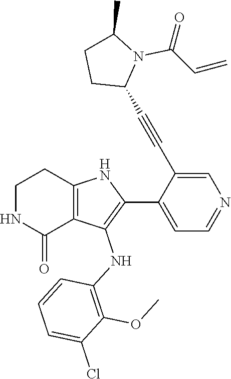 [SMILES: C=CC(=O)N1[C@H](C)CC[C@H]1C#Cc1cnccc1-c1[nH]c2c(c1Nc1cccc(Cl)c1OC)C(=O)NCC2] |
| 240 | 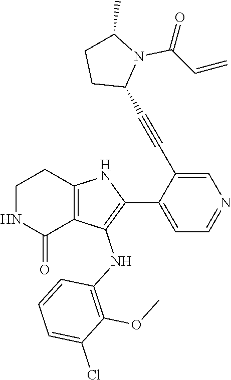 [SMILES: C=CC(=O)N1[C@H](C#Cc2cnccc2-c2[nH]c3c(c2Nc2cccc(Cl)c2OC)C(=O)NCC3)CC[C@@H]1C] |
| 241 | 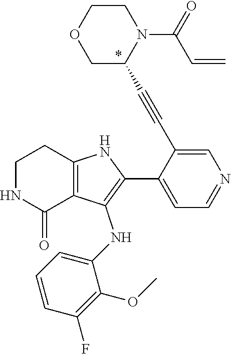 [SMILES: C=CC(=O)N1CCOC[C@H]1C#Cc1cnccc1-c1[nH]c2c(c1Nc1cccc(F)c1OC)C(=O)NCC2] |
| 242 | 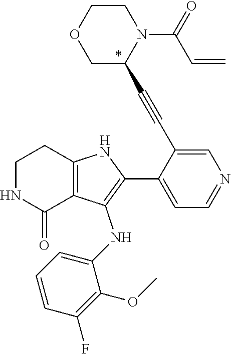 [SMILES: C=CC(=O)N1CCOC[C@@H]1C#Cc1cnccc1-c1[nH]c2c(c1Nc1cccc(F)c1OC)C(=O)NCC2] |
| 243 | 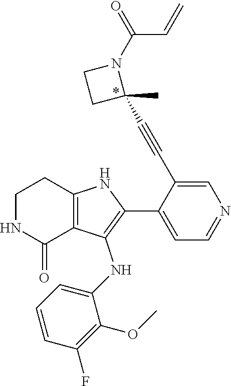 [SMILES: C=CC(=O)N1CC[C@@]1(C)C#Cc1cnccc1-c1[nH]c2c(c1Nc1cccc(F)c1OC)C(=O)NCC2] |
| 244 | 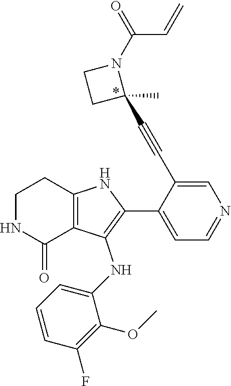 [SMILES: C=CC(=O)N1CC[C@]1(C)C#Cc1cnccc1-c1[nH]c2c(c1Nc1cccc(F)c1OC)C(=O)NCC2] |
| 245 | 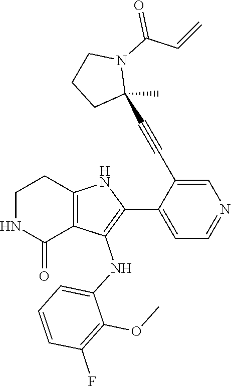 [SMILES: C=CC(=O)N1CCC[C@]1(C)C#Cc1cnccc1-c1[nH]c2c(c1Nc1cccc(F)c1OC)C(=O)NCC2] |
| 246 | 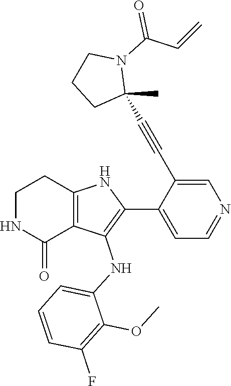 [SMILES: C=CC(=O)N1CCC[C@@]1(C)C#Cc1cnccc1-c1[nH]c2c(c1Nc1cccc(F)c1OC)C(=O)NCC2] |
| 247 | 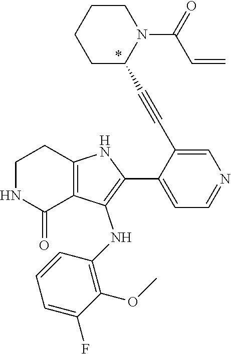 [SMILES: C=CC(=O)N1CCCC[C@H]1C#Cc1cnccc1-c1[nH]c2c(c1Nc1cccc(F)c1OC)C(=O)NCC2] |
| 248 | 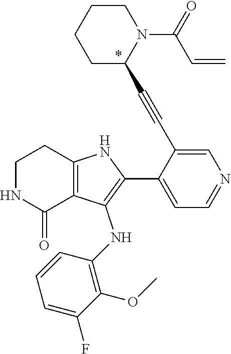 [SMILES: C=CC(=O)N1CCCC[C@@H]1C#Cc1cnccc1-c1[nH]c2c(c1Nc1cccc(F)c1OC)C(=O)NCC2] |
| 249 | 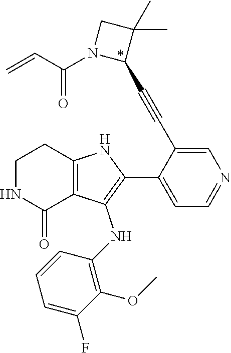 [SMILES: C=CC(=O)N1CC(C)(C)[C@H]1C#Cc1cnccc1-c1[nH]c2c(c1Nc1cccc(F)c1OC)C(=O)NCC2] |
| 250 | 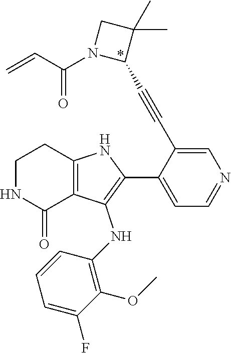 [SMILES: C=CC(=O)N1CC(C)(C)[C@@H]1C#Cc1cnccc1-c1[nH]c2c(c1Nc1cccc(F)c1OC)C(=O)NCC2] |
| 251 | 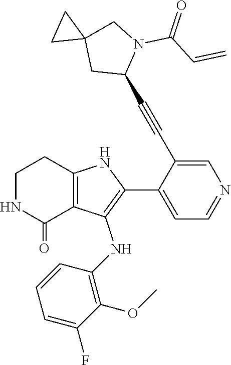 [SMILES: C=CC(=O)N1CC2(CC2)C[C@@H]1C#Cc1cnccc1-c1[nH]c2c(c1Nc1cccc(F)c1OC)C(=O)NCC2] |
| 252 | 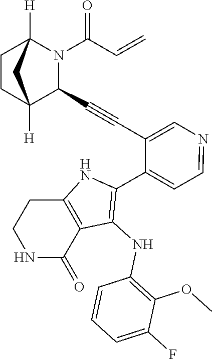 [SMILES: C=CC(=O)N1[C@H]2CC[C@H](C2)[C@@H]1C#Cc1cnccc1-c1[nH]c2c(c1Nc1cccc(F)c1OC)C(=O)NCC2] |
| 253 | 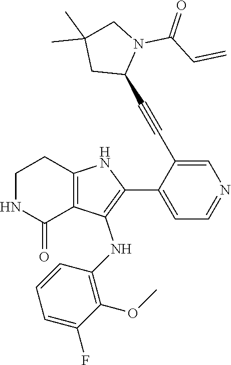 [SMILES: C=CC(=O)N1CC(C)(C)C[C@@H]1C#Cc1cnccc1-c1[nH]c2c(c1Nc1cccc(F)c1OC)C(=O)NCC2] |
| 254 | 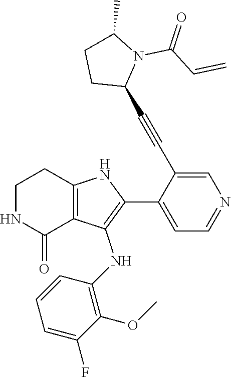 [SMILES: C=CC(=O)N1[C@@H](C)CC[C@@H]1C#Cc1cnccc1-c1[nH]c2c(c1Nc1cccc(F)c1OC)C(=O)NCC2] |
| 255 | 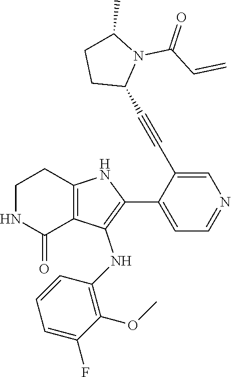 [SMILES: C=CC(=O)N1[C@H](C#Cc2cnccc2-c2[nH]c3c(c2Nc2cccc(F)c2OC)C(=O)NCC3)CC[C@@H]1C] |
| 256 | 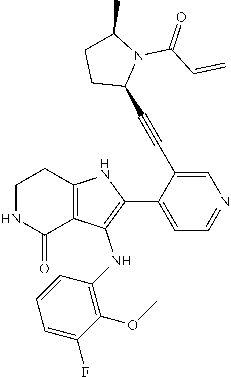 [SMILES: C=CC(=O)N1[C@H](C)CC[C@@H]1C#Cc1cnccc1-c1[nH]c2c(c1Nc1cccc(F)c1OC)C(=O)NCC2] |
| 257 | 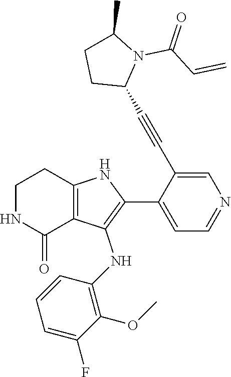 [SMILES: C=CC(=O)N1[C@H](C)CC[C@H]1C#Cc1cnccc1-c1[nH]c2c(c1Nc1cccc(F)c1OC)C(=O)NCC2] |
| 258 | 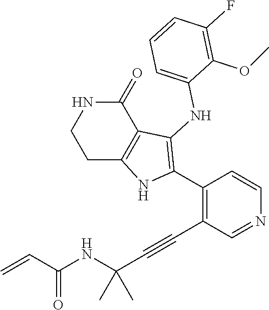 [SMILES: C=CC(=O)NC(C)(C)C#Cc1cnccc1-c1[nH]c2c(c1Nc1cccc(F)c1OC)C(=O)NCC2] |
| 259 | 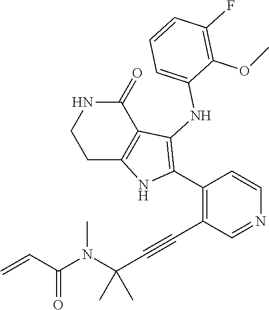 [SMILES: C=CC(=O)N(C)C(C)(C)C#Cc1cnccc1-c1[nH]c2c(c1Nc1cccc(F)c1OC)C(=O)NCC2] |
| 260 | 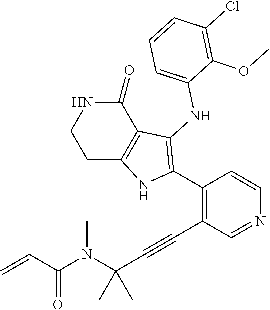 [SMILES: C=CC(=O)N(C)C(C)(C)C#Cc1cnccc1-c1[nH]c2c(c1Nc1cccc(Cl)c1OC)C(=O)NCC2] |
| 261 | 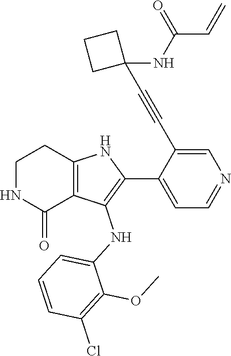 [SMILES: C=CC(=O)NC1(C#Cc2cnccc2-c2[nH]c3c(c2Nc2cccc(Cl)c2OC)C(=O)NCC3)CCC1] |
| 262 | 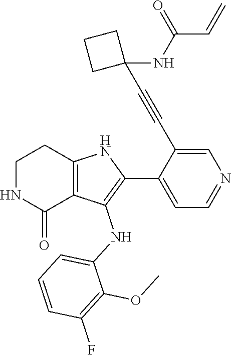 [SMILES: C=CC(=O)NC1(C#Cc2cnccc2-c2[nH]c3c(c2Nc2cccc(F)c2OC)C(=O)NCC3)CCC1] |
| 263 | 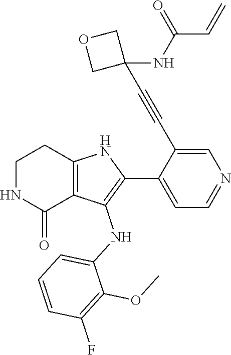 [SMILES: C=CC(=O)NC1(C#Cc2cnccc2-c2[nH]c3c(c2Nc2cccc(F)c2OC)C(=O)NCC3)COC1] |
| 264 | 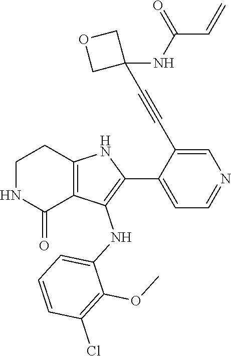 [SMILES: C=CC(=O)NC1(C#Cc2cnccc2-c2[nH]c3c(c2Nc2cccc(Cl)c2OC)C(=O)NCC3)COC1] |
| 265 | 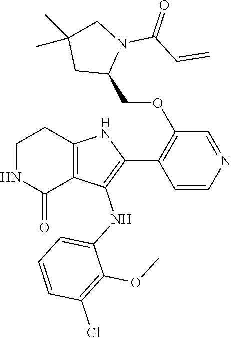 [SMILES: C=CC(=O)N1CC(C)(C)C[C@@H]1COc1cnccc1-c1[nH]c2c(c1Nc1cccc(Cl)c1OC)C(=O)NCC2] |
| 266 | 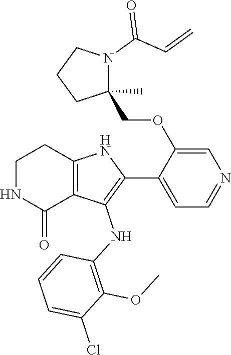 [SMILES: C=CC(=O)N1CCC[C@]1(C)COc1cnccc1-c1[nH]c2c(c1Nc1cccc(Cl)c1OC)C(=O)NCC2] |
| 267 | 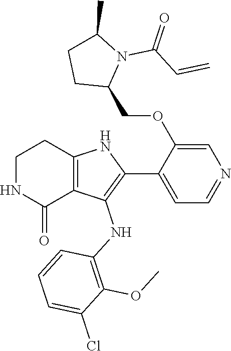 [SMILES: C=CC(=O)N1[C@@H](COc2cnccc2-c2[nH]c3c(c2Nc2cccc(Cl)c2OC)C(=O)NCC3)CC[C@H]1C] |
| 268 | 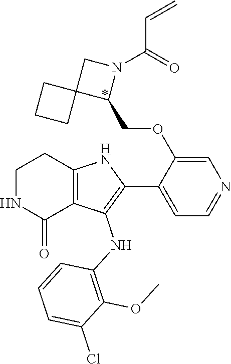 [SMILES: C=CC(=O)N1CC2(CCC2)[C@@H]1COc1cnccc1-c1[nH]c2c(c1Nc1cccc(Cl)c1OC)C(=O)NCC2] |
| 269 | 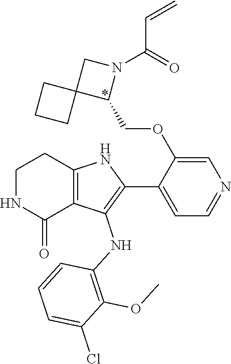 [SMILES: C=CC(=O)N1CC2(CCC2)[C@H]1COc1cnccc1-c1[nH]c2c(c1Nc1cccc(Cl)c1OC)C(=O)NCC2] |
| 270 | 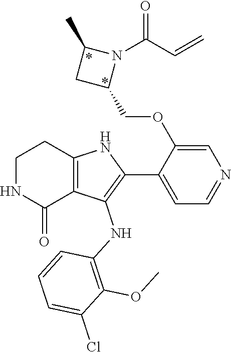 [SMILES: C=CC(=O)N1[C@H](COc2cnccc2-c2[nH]c3c(c2Nc2cccc(Cl)c2OC)C(=O)NCC3)C[C@H]1C] |
| 271 | 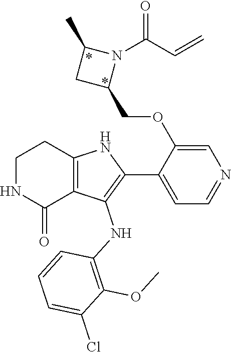 [SMILES: C=CC(=O)N1[C@@H](COc2cnccc2-c2[nH]c3c(c2Nc2cccc(Cl)c2OC)C(=O)NCC3)C[C@H]1C] |
| 272 | 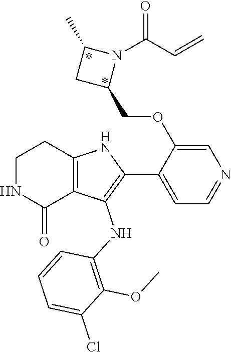 [SMILES: C=CC(=O)N1[C@@H](COc2cnccc2-c2[nH]c3c(c2Nc2cccc(Cl)c2OC)C(=O)NCC3)C[C@@H]1C] |
| 273 | 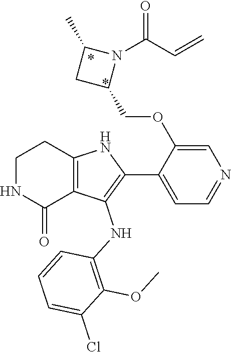 [SMILES: C=CC(=O)N1[C@H](COc2cnccc2-c2[nH]c3c(c2Nc2cccc(Cl)c2OC)C(=O)NCC3)C[C@@H]1C] |
| 274 | 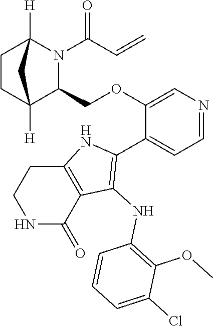 [SMILES: C=CC(=O)N1[C@H]2CC[C@H](C2)[C@@H]1COc1cnccc1-c1[nH]c2c(c1Nc1cccc(Cl)c1OC)C(=O)NCC2] |
| 275 | 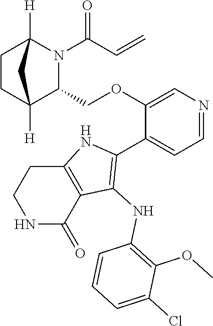 [SMILES: C=CC(=O)N1[C@H]2CC[C@H](C2)[C@H]1COc1cnccc1-c1[nH]c2c(c1Nc1cccc(Cl)c1OC)C(=O)NCC2] |
| 276 | 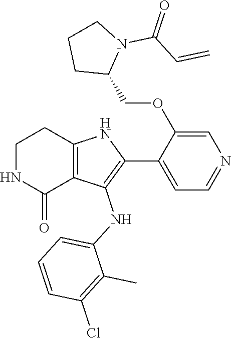 [SMILES: C=CC(=O)N1CCC[C@H]1COc1cnccc1-c1[nH]c2c(c1Nc1cccc(Cl)c1C)C(=O)NCC2] |
| 277 | 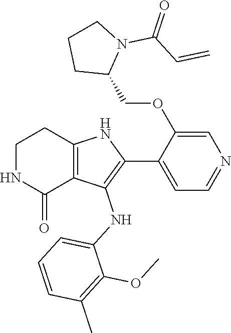 [SMILES: C=CC(=O)N1CCC[C@H]1COc1cnccc1-c1[nH]c2c(c1Nc1cccc(C)c1OC)C(=O)NCC2] |
| 278 | 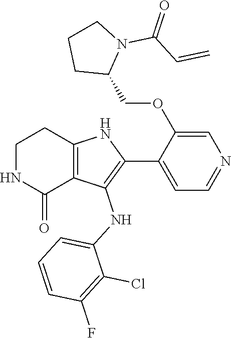 [SMILES: C=CC(=O)N1CCC[C@H]1COc1cnccc1-c1[nH]c2c(c1Nc1cccc(F)c1Cl)C(=O)NCC2] |
| 279 | 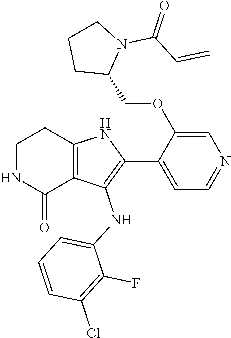 [SMILES: C=CC(=O)N1CCC[C@H]1COc1cnccc1-c1[nH]c2c(c1Nc1cccc(Cl)c1F)C(=O)NCC2] |
| 280 | 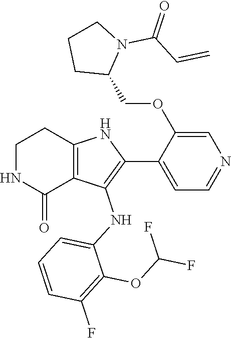 [SMILES: C=CC(=O)N1CCC[C@H]1COc1cnccc1-c1[nH]c2c(c1Nc1cccc(F)c1OC(F)F)C(=O)NCC2] |
| 281 | 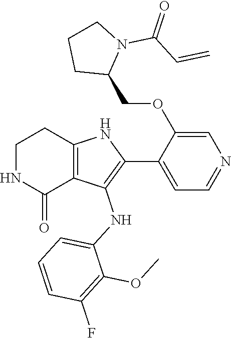 [SMILES: C=CC(=O)N1CCC[C@@H]1COc1cnccc1-c1[nH]c2c(c1Nc1cccc(F)c1OC)C(=O)NCC2] |
| 282 | 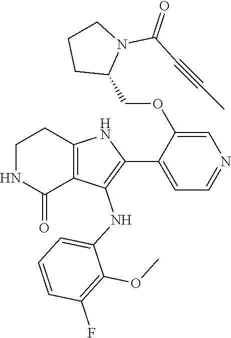 [SMILES: CC#CC(=O)N1CCC[C@H]1COc1cnccc1-c1[nH]c2c(c1Nc1cccc(F)c1OC)C(=O)NCC2] |
| 283 | 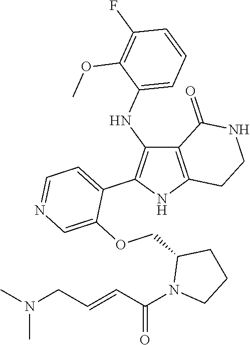 [SMILES: COc1c(F)cccc1Nc1c(-c2ccncc2OC[C@@H]2CCCN2C(=O)/C=C/CN(C)C)[nH]c2c1C(=O)NCC2] |
| 284 | 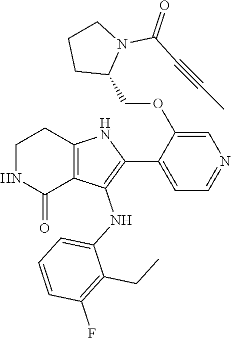 [SMILES: CC#CC(=O)N1CCC[C@H]1COc1cnccc1-c1[nH]c2c(c1Nc1cccc(F)c1CC)C(=O)NCC2] |
| 285 | 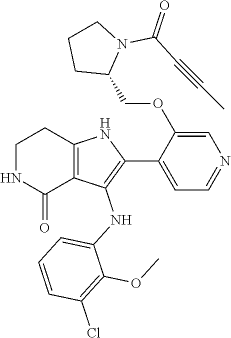 [SMILES: CC#CC(=O)N1CCC[C@H]1COc1cnccc1-c1[nH]c2c(c1Nc1cccc(Cl)c1OC)C(=O)NCC2] |
| 286 | 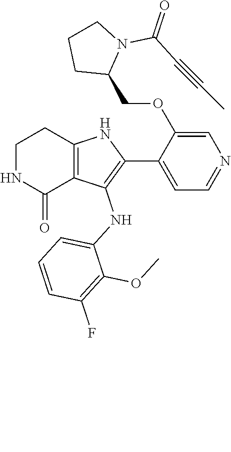 [SMILES: CC#CC(=O)N1CCC[C@@H]1COc1cnccc1-c1[nH]c2c(c1Nc1cccc(F)c1OC)C(=O)NCC2] |
| 287 | 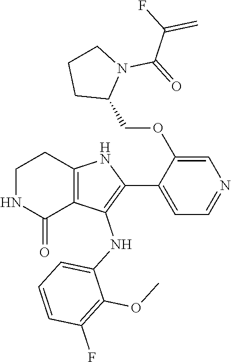 [SMILES: C=C(F)C(=O)N1CCC[C@H]1COc1cnccc1-c1[nH]c2c(c1Nc1cccc(F)c1OC)C(=O)NCC2] |
| 288 | 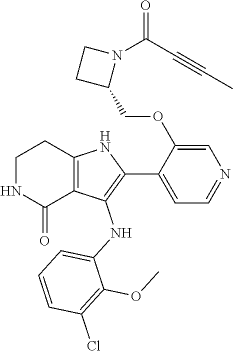 [SMILES: CC#CC(=O)N1CC[C@H]1COc1cnccc1-c1[nH]c2c(c1Nc1cccc(Cl)c1OC)C(=O)NCC2] |
| 289 | 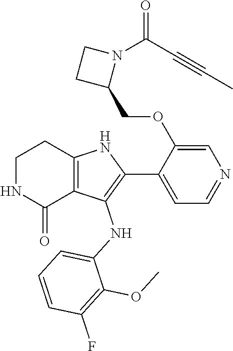 [SMILES: CC#CC(=O)N1CC[C@@H]1COc1cnccc1-c1[nH]c2c(c1Nc1cccc(F)c1OC)C(=O)NCC2] |
| 290 | 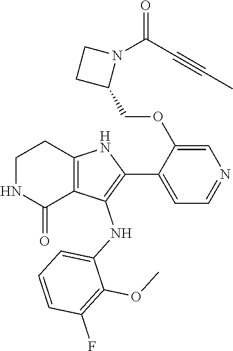 [SMILES: CC#CC(=O)N1CC[C@H]1COc1cnccc1-c1[nH]c2c(c1Nc1cccc(F)c1OC)C(=O)NCC2] |
| 291 | 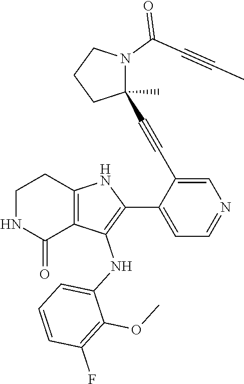 [SMILES: CC#CC(=O)N1CCC[C@]1(C)C#Cc1cnccc1-c1[nH]c2c(c1Nc1cccc(F)c1OC)C(=O)NCC2] |
| 292 | 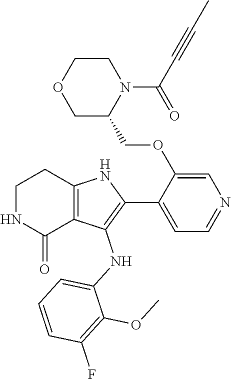 [SMILES: CC#CC(=O)N1CCOC[C@H]1COc1cnccc1-c1[nH]c2c(c1Nc1cccc(F)c1OC)C(=O)NCC2] |
| 293 | 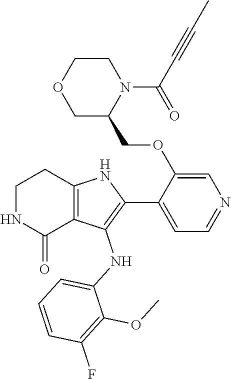 [SMILES: CC#CC(=O)N1CCOC[C@@H]1COc1cnccc1-c1[nH]c2c(c1Nc1cccc(F)c1OC)C(=O)NCC2] |
| 294 | 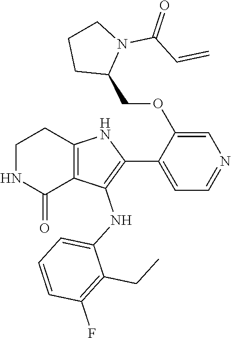 [SMILES: C=CC(=O)N1CCC[C@@H]1COc1cnccc1-c1[nH]c2c(c1Nc1cccc(F)c1CC)C(=O)NCC2] |
| 295 | 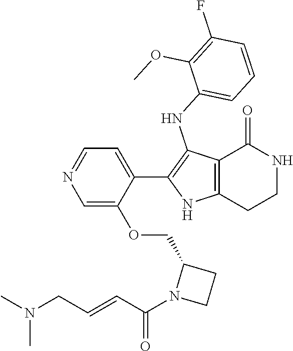 [SMILES: COc1c(F)cccc1Nc1c(-c2ccncc2OC[C@@H]2CCN2C(=O)/C=C/CN(C)C)[nH]c2c1C(=O)NCC2] |
| 296 | 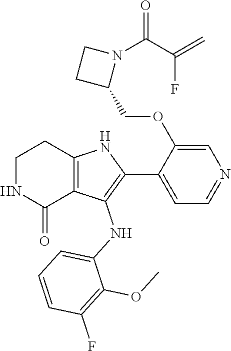 [SMILES: C=C(F)C(=O)N1CC[C@H]1COc1cnccc1-c1[nH]c2c(c1Nc1cccc(F)c1OC)C(=O)NCC2] |
| 297 | 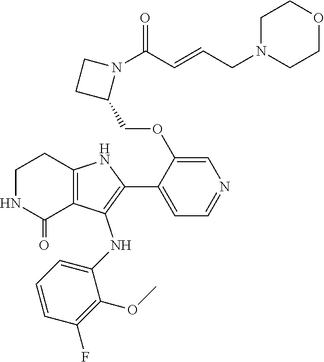 [SMILES: COc1c(F)cccc1Nc1c(-c2ccncc2OC[C@@H]2CCN2C(=O)/C=C/CN2CCOCC2)[nH]c2c1C(=O)NCC2] |
| 298 | 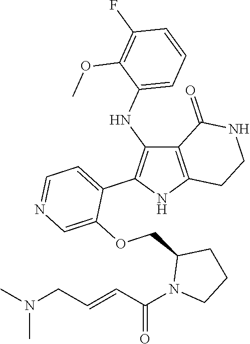 [SMILES: COc1c(F)cccc1Nc1c(-c2ccncc2OC[C@H]2CCCN2C(=O)/C=C/CN(C)C)[nH]c2c1C(=O)NCC2] |
| 299 | 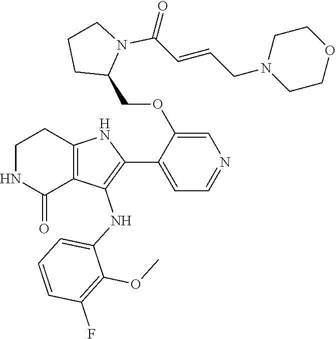 [SMILES: COc1c(F)cccc1Nc1c(-c2ccncc2OC[C@H]2CCCN2C(=O)/C=C/CN2CCOCC2)[nH]c2c1C(=O)NCC2] |
| 300 | 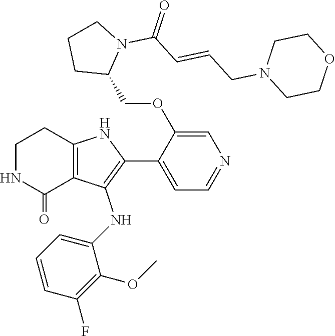 [SMILES: COc1c(F)cccc1Nc1c(-c2ccncc2OC[C@@H]2CCCN2C(=O)/C=C/CN2CCOCC2)[nH]c2c1C(=O)NCC2] |
| 301 | 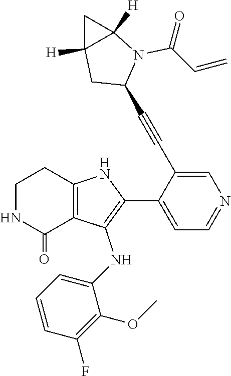 [SMILES: C=CC(=O)N1[C@@H](C#Cc2cnccc2-c2[nH]c3c(c2Nc2cccc(F)c2OC)C(=O)NCC3)C[C@@H]2C[C@@H]21] |
| 302 | 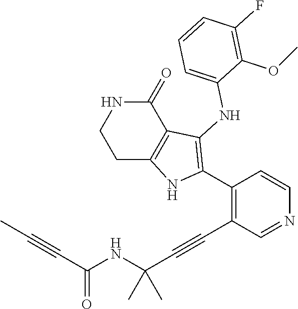 [SMILES: CC#CC(=O)NC(C)(C)C#Cc1cnccc1-c1[nH]c2c(c1Nc1cccc(F)c1OC)C(=O)NCC2] |
| 303 | 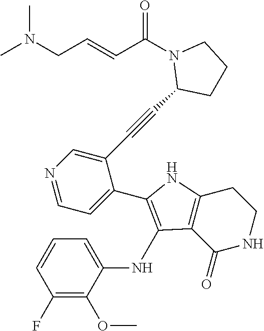 [SMILES: COc1c(F)cccc1Nc1c(-c2ccncc2C#C[C@H]2CCCN2C(=O)/C=C/CN(C)C)[nH]c2c1C(=O)NCC2] |
| 304 | 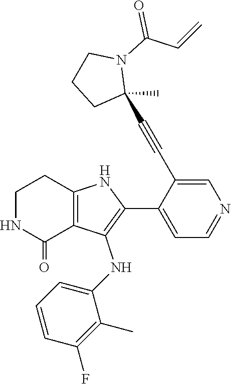 [SMILES: C=CC(=O)N1CCC[C@]1(C)C#Cc1cnccc1-c1[nH]c2c(c1Nc1cccc(F)c1C)C(=O)NCC2] |
| 305 | 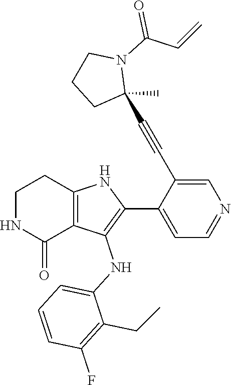 [SMILES: C=CC(=O)N1CCC[C@]1(C)C#Cc1cnccc1-c1[nH]c2c(c1Nc1cccc(F)c1CC)C(=O)NCC2] |
| 306 | 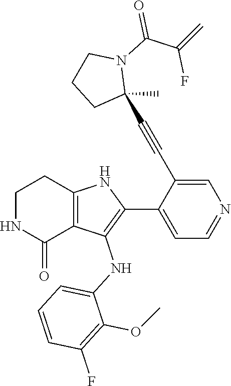 [SMILES: C=C(F)C(=O)N1CCC[C@]1(C)C#Cc1cnccc1-c1[nH]c2c(c1Nc1cccc(F)c1OC)C(=O)NCC2] |
| 307 | 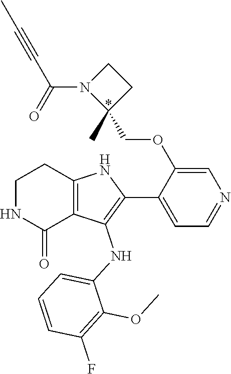 [SMILES: CC#CC(=O)N1CC[C@]1(C)COc1cnccc1-c1[nH]c2c(c1Nc1cccc(F)c1OC)C(=O)NCC2] |
| 308 | 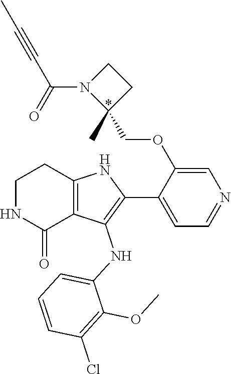 [SMILES: CC#CC(=O)N1CC[C@]1(C)COc1cnccc1-c1[nH]c2c(c1Nc1cccc(Cl)c1OC)C(=O)NCC2] |
| 309 | 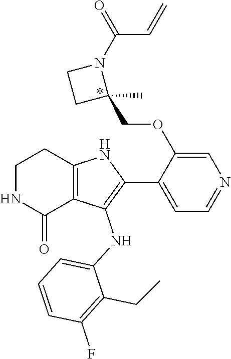 [SMILES: C=CC(=O)N1CC[C@]1(C)COc1cnccc1-c1[nH]c2c(c1Nc1cccc(F)c1CC)C(=O)NCC2] |
| 310 | 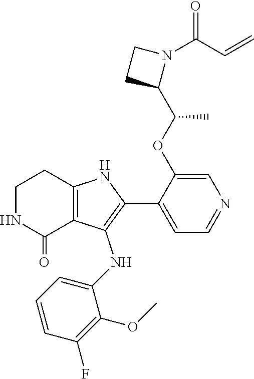 [SMILES: C=CC(=O)N1CC[C@@H]1[C@H](C)Oc1cnccc1-c1[nH]c2c(c1Nc1cccc(F)c1OC)C(=O)NCC2] |
| 311 | 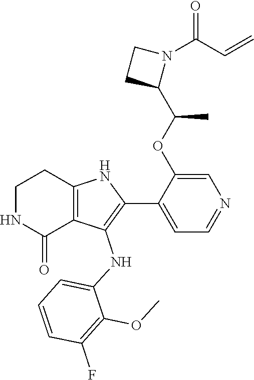 [SMILES: C=CC(=O)N1CC[C@@H]1[C@@H](C)Oc1cnccc1-c1[nH]c2c(c1Nc1cccc(F)c1OC)C(=O)NCC2] |
| 312 | 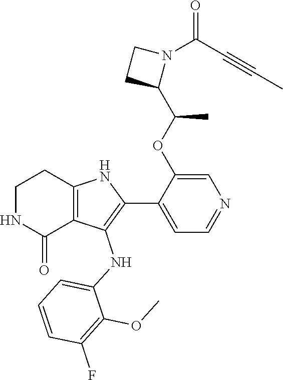 [SMILES: CC#CC(=O)N1CC[C@@H]1[C@@H](C)Oc1cnccc1-c1[nH]c2c(c1Nc1cccc(F)c1OC)C(=O)NCC2] |
| 313 | 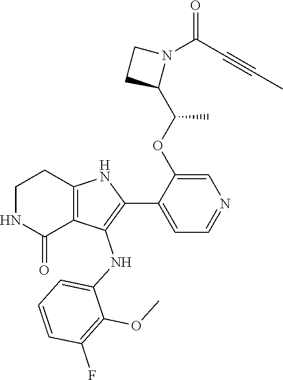 [SMILES: CC#CC(=O)N1CC[C@@H]1[C@H](C)Oc1cnccc1-c1[nH]c2c(c1Nc1cccc(F)c1OC)C(=O)NCC2] |
| 314 | 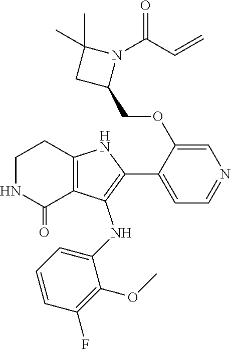 [SMILES: C=CC(=O)N1[C@@H](COc2cnccc2-c2[nH]c3c(c2Nc2cccc(F)c2OC)C(=O)NCC3)CC1(C)C] |
| 315 | 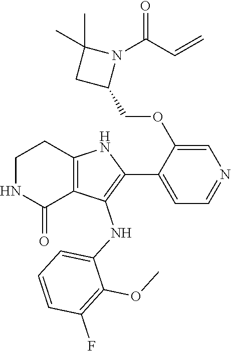 [SMILES: C=CC(=O)N1[C@H](COc2cnccc2-c2[nH]c3c(c2Nc2cccc(F)c2OC)C(=O)NCC3)CC1(C)C] |
| 316 | 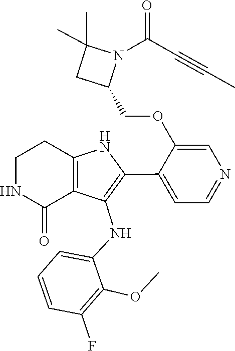 [SMILES: CC#CC(=O)N1[C@H](COc2cnccc2-c2[nH]c3c(c2Nc2cccc(F)c2OC)C(=O)NCC3)CC1(C)C] |
| 317 | 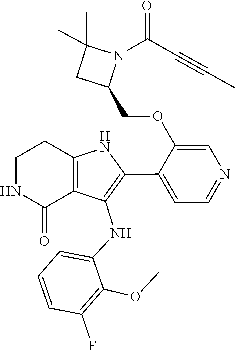 [SMILES: CC#CC(=O)N1[C@@H](COc2cnccc2-c2[nH]c3c(c2Nc2cccc(F)c2OC)C(=O)NCC3)CC1(C)C] |
| 318 | 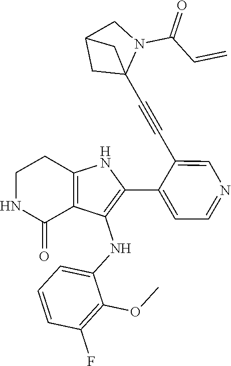 [SMILES: C=CC(=O)N1CC2CC1(C#Cc1cnccc1-c1[nH]c3c(c1Nc1cccc(F)c1OC)C(=O)NCC3)C2] |
| 319 | 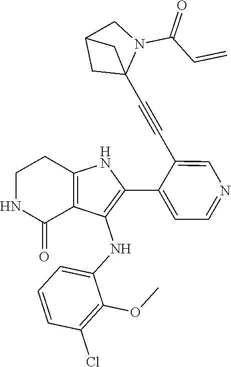 [SMILES: C=CC(=O)N1CC2CC1(C#Cc1cnccc1-c1[nH]c3c(c1Nc1cccc(Cl)c1OC)C(=O)NCC3)C2] |
| 320 | 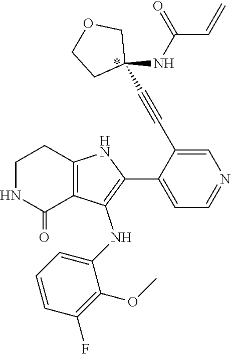 [SMILES: C=CC(=O)N[C@@]1(C#Cc2cnccc2-c2[nH]c3c(c2Nc2cccc(F)c2OC)C(=O)NCC3)CCOC1] |
| 321 | 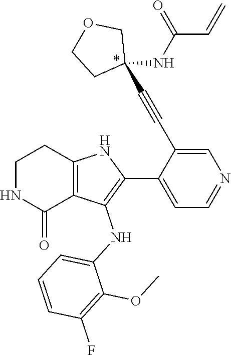 [SMILES: C=CC(=O)N[C@]1(C#Cc2cnccc2-c2[nH]c3c(c2Nc2cccc(F)c2OC)C(=O)NCC3)CCOC1] |
| 322 | 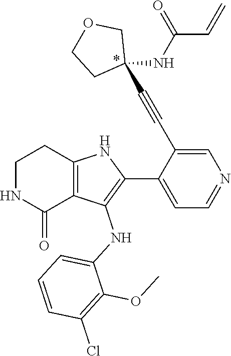 [SMILES: C=CC(=O)N[C@]1(C#Cc2cnccc2-c2[nH]c3c(c2Nc2cccc(Cl)c2OC)C(=O)NCC3)CCOC1] |
| 323 | 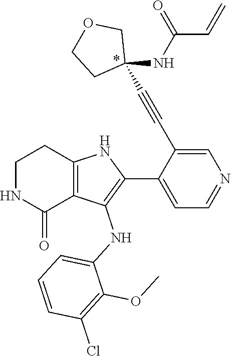 [SMILES: C=CC(=O)N[C@@]1(C#Cc2cnccc2-c2[nH]c3c(c2Nc2cccc(Cl)c2OC)C(=O)NCC3)CCOC1] |
| 324 | 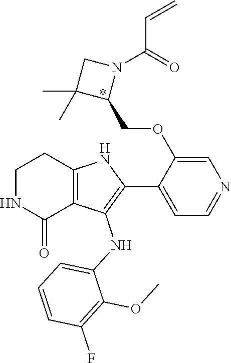 [SMILES: C=CC(=O)N1CC(C)(C)[C@@H]1COc1cnccc1-c1[nH]c2c(c1Nc1cccc(F)c1OC)C(=O)NCC2] |
| 325 | 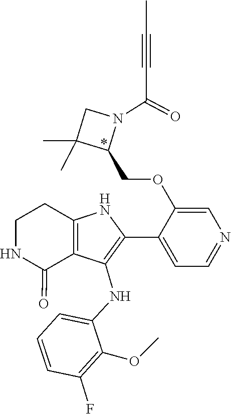 [SMILES: CC#CC(=O)N1CC(C)(C)[C@@H]1COc1cnccc1-c1[nH]c2c(c1Nc1cccc(F)c1OC)C(=O)NCC2] |
| 326 | 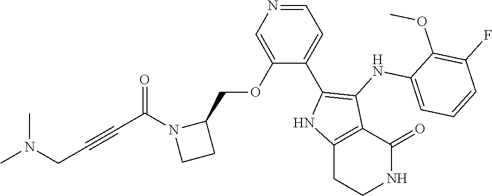 [SMILES: COc1c(F)cccc1Nc1c(-c2ccncc2OC[C@H]2CCN2C(=O)C#CCN(C)C)[nH]c2c1C(=O)NCC2] |
| 327 | 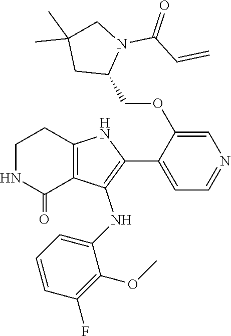 [SMILES: C=CC(=O)N1CC(C)(C)C[C@H]1COc1cnccc1-c1[nH]c2c(c1Nc1cccc(F)c1OC)C(=O)NCC2] |
| 328 | 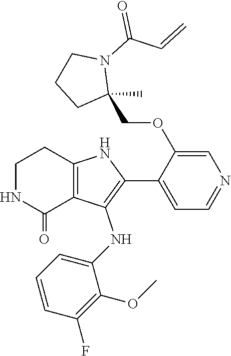 [SMILES: C=CC(=O)N1CCC[C@]1(C)COc1cnccc1-c1[nH]c2c(c1Nc1cccc(F)c1OC)C(=O)NCC2] |
| 329 | 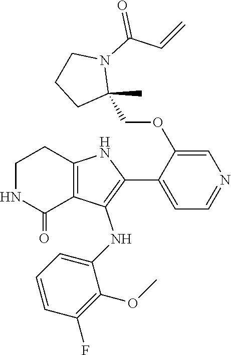 [SMILES: C=CC(=O)N1CCC[C@@]1(C)COc1cnccc1-c1[nH]c2c(c1Nc1cccc(F)c1OC)C(=O)NCC2] |
| 330 | 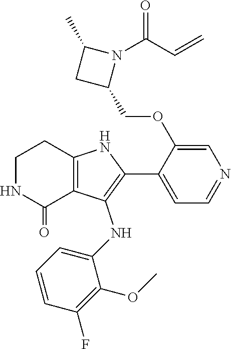 [SMILES: C=CC(=O)N1[C@H](COc2cnccc2-c2[nH]c3c(c2Nc2cccc(F)c2OC)C(=O)NCC3)C[C@@H]1C] |
| 331 | 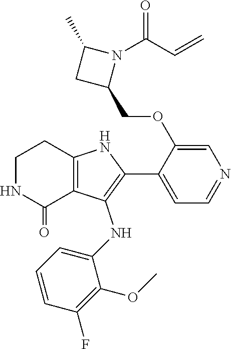 [SMILES: C=CC(=O)N1[C@@H](COc2cnccc2-c2[nH]c3c(c2Nc2cccc(F)c2OC)C(=O)NCC3)C[C@@H]1C] |
| 332 | 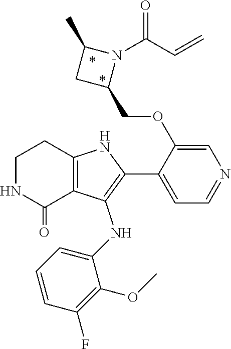 [SMILES: C=CC(=O)N1[C@@H](COc2cnccc2-c2[nH]c3c(c2Nc2cccc(F)c2OC)C(=O)NCC3)C[C@H]1C] |
| 333 | 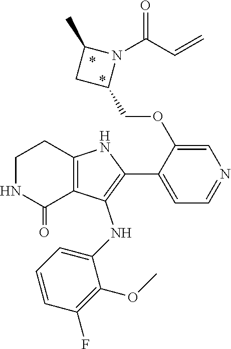 [SMILES: C=CC(=O)N1[C@H](COc2cnccc2-c2[nH]c3c(c2Nc2cccc(F)c2OC)C(=O)NCC3)C[C@H]1C] |
| 334 | 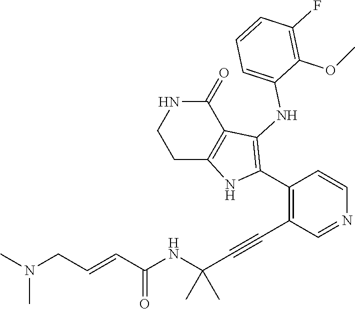 |
| 335 | 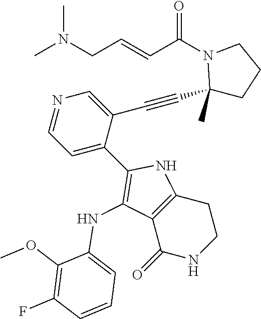 [SMILES: COc1c(F)cccc1Nc1c(-c2ccncc2C#C[C@@]2(C)CCCN2C(=O)/C=C/CN(C)C)[nH]c2c1C(=O)NCC2] |
| 336 | 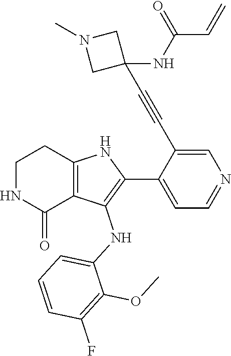 [SMILES: C=CC(=O)NC1(C#Cc2cnccc2-c2[nH]c3c(c2Nc2cccc(F)c2OC)C(=O)NCC3)CN(C)C1] |
| 337 | 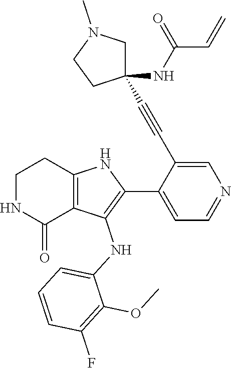 [SMILES: C=CC(=O)N[C@@]1(C#Cc2cnccc2-c2[nH]c3c(c2Nc2cccc(F)c2OC)C(=O)NCC3)CCN(C)C1] |
| 338 | 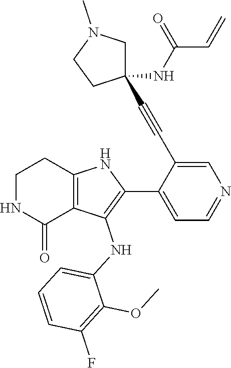 [SMILES: C=CC(=O)N[C@]1(C#Cc2cnccc2-c2[nH]c3c(c2Nc2cccc(F)c2OC)C(=O)NCC3)CCN(C)C1] |
| 339 | 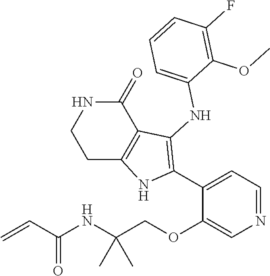 [SMILES: C=CC(=O)NC(C)(C)COc1cnccc1-c1[nH]c2c(c1Nc1cccc(F)c1OC)C(=O)NCC2] |
| 340 | 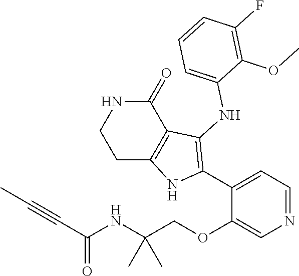 [SMILES: CC#CC(=O)NC(C)(C)COc1cnccc1-c1[nH]c2c(c1Nc1cccc(F)c1OC)C(=O)NCC2] |
| 341 | 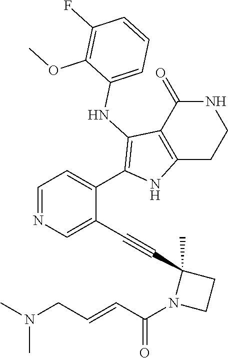 [SMILES: COc1c(F)cccc1Nc1c(-c2ccncc2C#C[C@@]2(C)CCN2C(=O)/C=C/CN(C)C)[nH]c2c1C(=O)NCC2] |
| 342 | 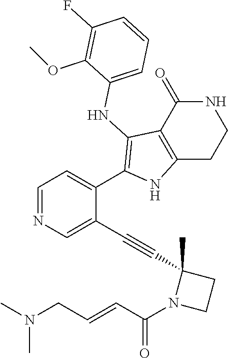 [SMILES: COc1c(F)cccc1Nc1c(-c2ccncc2C#C[C@]2(C)CCN2C(=O)/C=C/CN(C)C)[nH]c2c1C(=O)NCC2] |
| 343 | 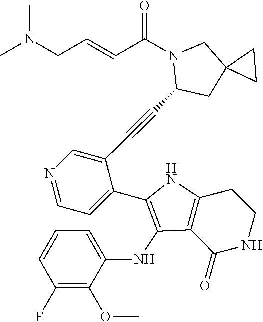 [SMILES: COc1c(F)cccc1Nc1c(-c2ccncc2C#C[C@H]2CC3(CC3)CN2C(=O)/C=C/CN(C)C)[nH]c2c1C(=O)NCC2] |
| 344 | 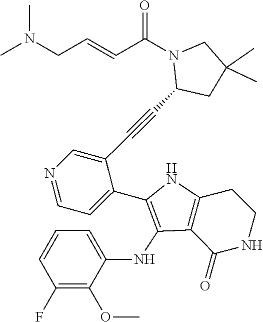 |
| 345 | 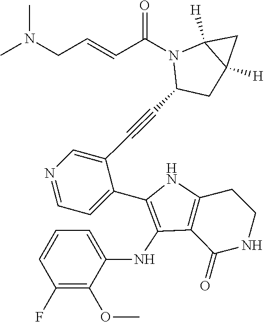 [SMILES: COc1c(F)cccc1Nc1c(-c2ccncc2C#C[C@H]2C[C@@H]3C[C@@H]3N2C(=O)/C=C/CN(C)C)[nH]c2c1C(=O)NCC2] |
| 346 | 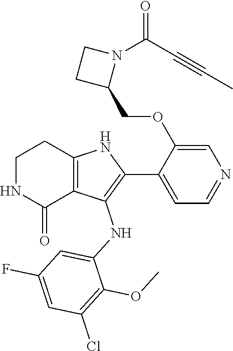 [SMILES: CC#CC(=O)N1CC[C@@H]1COc1cnccc1-c1[nH]c2c(c1Nc1cc(F)cc(Cl)c1OC)C(=O)NCC2] |
| 347 | 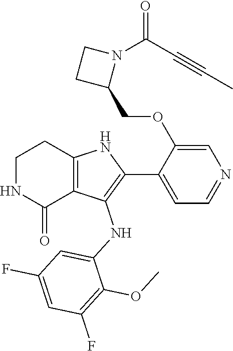 [SMILES: CC#CC(=O)N1CC[C@@H]1COc1cnccc1-c1[nH]c2c(c1Nc1cc(F)cc(F)c1OC)C(=O)NCC2] |
| 348 | 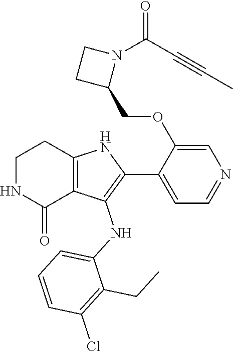 [SMILES: CC#CC(=O)N1CC[C@@H]1COc1cnccc1-c1[nH]c2c(c1Nc1cccc(Cl)c1CC)C(=O)NCC2] |
| 349 | 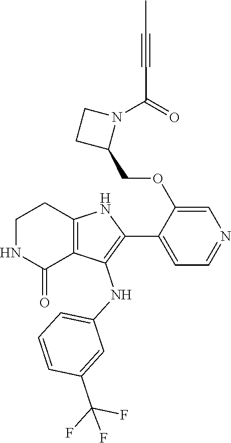 [SMILES: CC#CC(=O)N1CC[C@@H]1COc1cnccc1-c1[nH]c2c(c1Nc1cccc(C(F)(F)F)c1)C(=O)NCC2] |
| 350 | 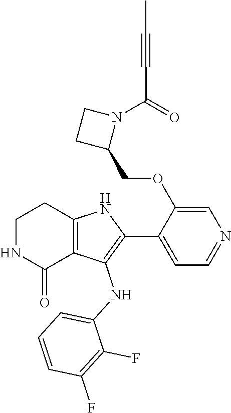 [SMILES: CC#CC(=O)N1CC[C@@H]1COc1cnccc1-c1[nH]c2c(c1Nc1cccc(F)c1F)C(=O)NCC2] |
| 351 | 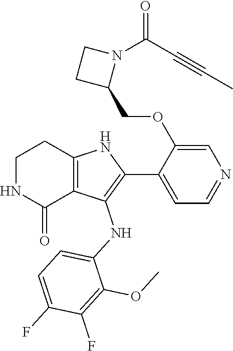 [SMILES: CC#CC(=O)N1CC[C@@H]1COc1cnccc1-c1[nH]c2c(c1Nc1ccc(F)c(F)c1OC)C(=O)NCC2] |
| 352 | 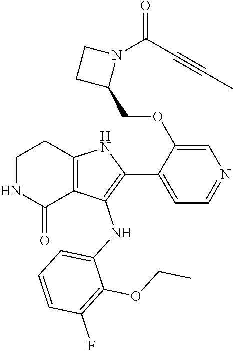 [SMILES: CC#CC(=O)N1CC[C@@H]1COc1cnccc1-c1[nH]c2c(c1Nc1cccc(F)c1OCC)C(=O)NCC2] |
| 353 | 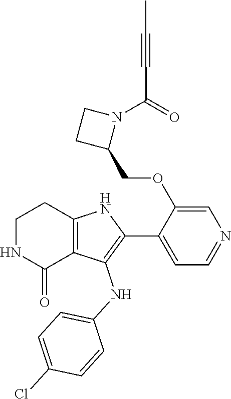 [SMILES: CC#CC(=O)N1CC[C@@H]1COc1cnccc1-c1[nH]c2c(c1Nc1ccc(Cl)cc1)C(=O)NCC2] |
| 354 | 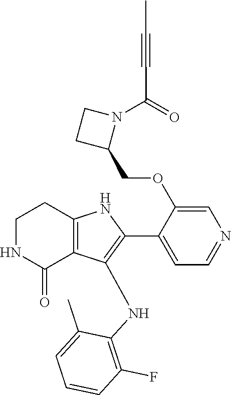 [SMILES: CC#CC(=O)N1CC[C@@H]1COc1cnccc1-c1[nH]c2c(c1Nc1c(C)cccc1F)C(=O)NCC2] |
| 355 | 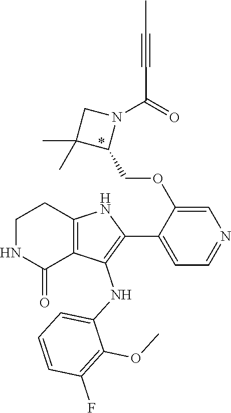 [SMILES: CC#CC(=O)N1CC(C)(C)[C@H]1COc1cnccc1-c1[nH]c2c(c1Nc1cccc(F)c1OC)C(=O)NCC2] |
| 356 | 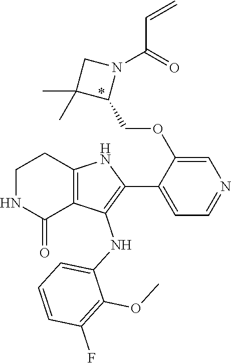 [SMILES: C=CC(=O)N1CC(C)(C)[C@H]1COc1cnccc1-c1[nH]c2c(c1Nc1cccc(F)c1OC)C(=O)NCC2] |
| 357 | 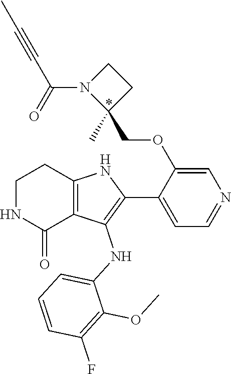 [SMILES: CC#CC(=O)N1CC[C@@]1(C)COc1cnccc1-c1[nH]c2c(c1Nc1cccc(F)c1OC)C(=O)NCC2] |
| 358 | 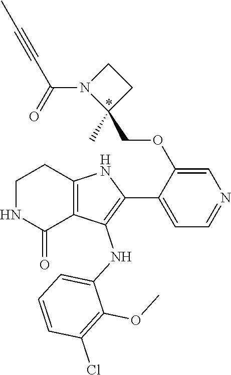 [SMILES: CC#CC(=O)N1CC[C@@]1(C)COc1cnccc1-c1[nH]c2c(c1Nc1cccc(Cl)c1OC)C(=O)NCC2] |
| 359 | 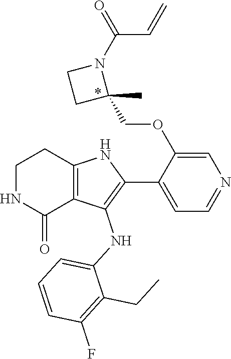 [SMILES: C=CC(=O)N1CC[C@@]1(C)COc1cnccc1-c1[nH]c2c(c1Nc1cccc(F)c1CC)C(=O)NCC2] |
| 360 | 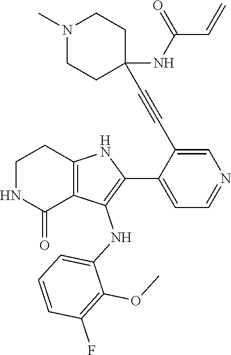 [SMILES: C=CC(=O)NC1(C#Cc2cnccc2-c2[nH]c3c(c2Nc2cccc(F)c2OC)C(=O)NCC3)CCN(C)CC1] |
| 361 | 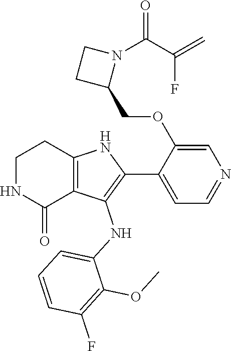 [SMILES: C=C(F)C(=O)N1CC[C@@H]1COc1cnccc1-c1[nH]c2c(c1Nc1cccc(F)c1OC)C(=O)NCC2] |
| 362 | 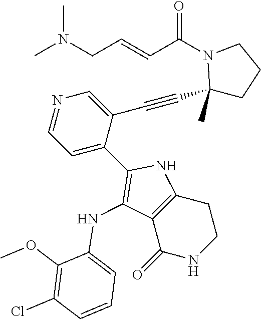 [SMILES: COc1c(Cl)cccc1Nc1c(-c2ccncc2C#C[C@@]2(C)CCCN2C(=O)/C=C/CN(C)C)[nH]c2c1C(=O)NCC2] |
| 363 | 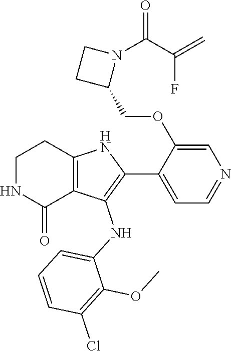 [SMILES: C=C(F)C(=O)N1CC[C@H]1COc1cnccc1-c1[nH]c2c(c1Nc1cccc(Cl)c1OC)C(=O)NCC2] |
| 364 | 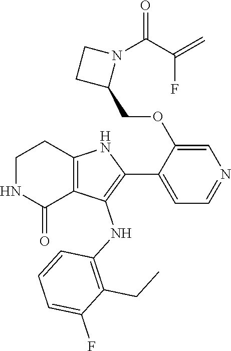 [SMILES: C=C(F)C(=O)N1CC[C@@H]1COc1cnccc1-c1[nH]c2c(c1Nc1cccc(F)c1CC)C(=O)NCC2] |
| 365 | 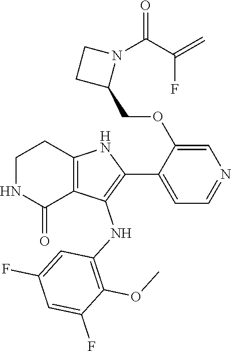 [SMILES: C=C(F)C(=O)N1CC[C@@H]1COc1cnccc1-c1[nH]c2c(c1Nc1cc(F)cc(F)c1OC)C(=O)NCC2] |
| 366 | 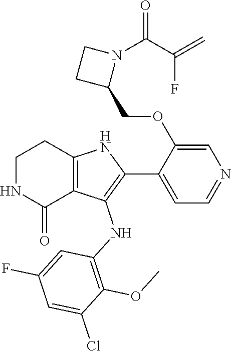 [SMILES: C=C(F)C(=O)N1CC[C@@H]1COc1cnccc1-c1[nH]c2c(c1Nc1cc(F)cc(Cl)c1OC)C(=O)NCC2] |
| 367 | 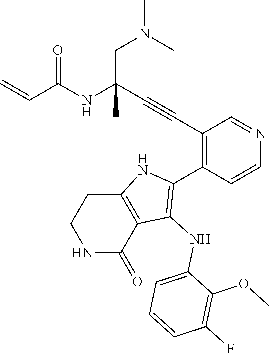 [SMILES: C=CC(=O)N[C@@](C)(C#Cc1cnccc1-c1[nH]c2c(c1Nc1cccc(F)c1OC)C(=O)NCC2)CN(C)C] |
| 368 | 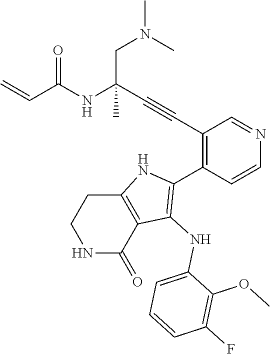 [SMILES: C=CC(=O)N[C@](C)(C#Cc1cnccc1-c1[nH]c2c(c1Nc1cccc(F)c1OC)C(=O)NCC2)CN(C)C] |
| 369 | 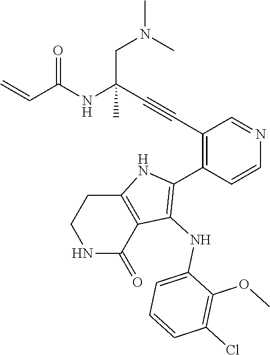 [SMILES: C=CC(=O)N[C@](C)(C#Cc1cnccc1-c1[nH]c2c(c1Nc1cccc(Cl)c1OC)C(=O)NCC2)CN(C)C] |
| 370 | 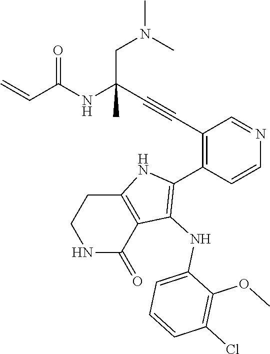 [SMILES: C=CC(=O)N[C@@](C)(C#Cc1cnccc1-c1[nH]c2c(c1Nc1cccc(Cl)c1OC)C(=O)NCC2)CN(C)C] |
| 371 | 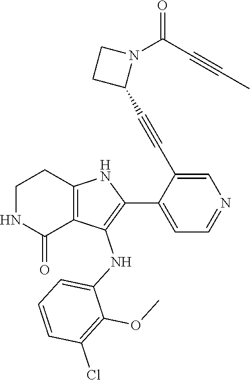 [SMILES: CC#CC(=O)N1CC[C@H]1C#Cc1cnccc1-c1[nH]c2c(c1Nc1cccc(Cl)c1OC)C(=O)NCC2] |
| 372 | 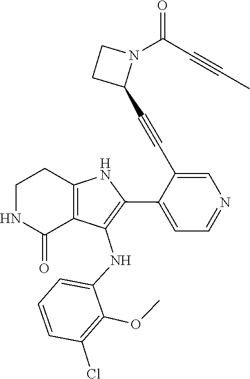 [SMILES: CC#CC(=O)N1CC[C@@H]1C#Cc1cnccc1-c1[nH]c2c(c1Nc1cccc(Cl)c1OC)C(=O)NCC2] |
| 373 | 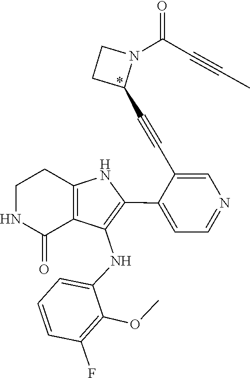 [SMILES: CC#CC(=O)N1CC[C@@H]1C#Cc1cnccc1-c1[nH]c2c(c1Nc1cccc(F)c1OC)C(=O)NCC2] |
| 374 | 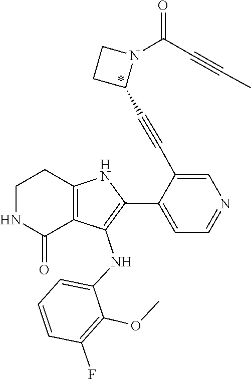 [SMILES: CC#CC(=O)N1CC[C@H]1C#Cc1cnccc1-c1[nH]c2c(c1Nc1cccc(F)c1OC)C(=O)NCC2] |
| 375 | 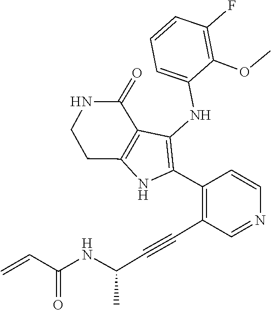 [SMILES: C=CC(=O)N[C@@H](C)C#Cc1cnccc1-c1[nH]c2c(c1Nc1cccc(F)c1OC)C(=O)NCC2] |
| 376 | 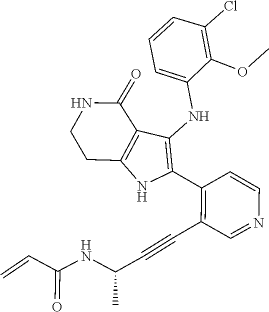 [SMILES: C=CC(=O)N[C@@H](C)C#Cc1cnccc1-c1[nH]c2c(c1Nc1cccc(Cl)c1OC)C(=O)NCC2] |
| 377 | 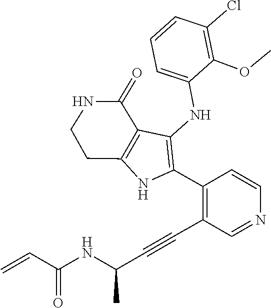 [SMILES: C=CC(=O)N[C@H](C)C#Cc1cnccc1-c1[nH]c2c(c1Nc1cccc(Cl)c1OC)C(=O)NCC2] |
| 378 | 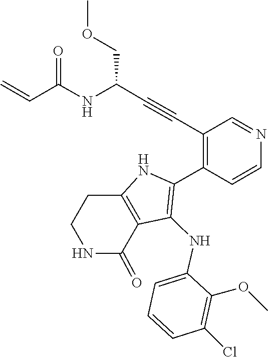 [SMILES: C=CC(=O)N[C@@H](C#Cc1cnccc1-c1[nH]c2c(c1Nc1cccc(Cl)c1OC)C(=O)NCC2)COC] |
| 379 | 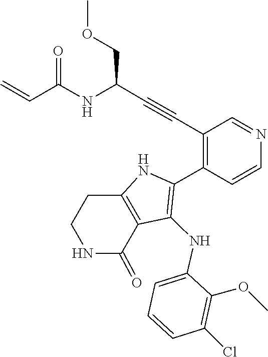 [SMILES: C=CC(=O)N[C@H](C#Cc1cnccc1-c1[nH]c2c(c1Nc1cccc(Cl)c1OC)C(=O)NCC2)COC] |
| 380 | 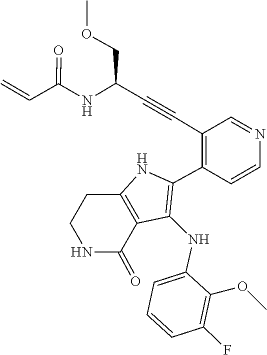 [SMILES: C=CC(=O)N[C@H](C#Cc1cnccc1-c1[nH]c2c(c1Nc1cccc(F)c1OC)C(=O)NCC2)COC] |
| 381 | 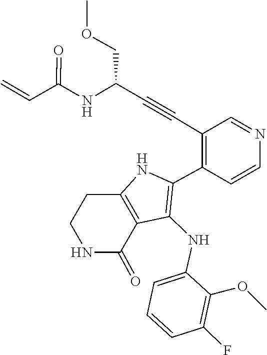 [SMILES: C=CC(=O)N[C@@H](C#Cc1cnccc1-c1[nH]c2c(c1Nc1cccc(F)c1OC)C(=O)NCC2)COC] |
| 382 | 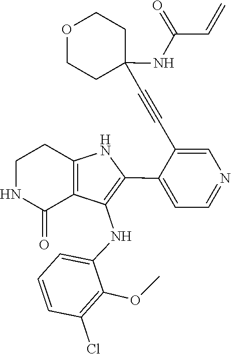 [SMILES: C=CC(=O)NC1(C#Cc2cnccc2-c2[nH]c3c(c2Nc2cccc(Cl)c2OC)C(=O)NCC3)CCOCC1] |
| 383 | 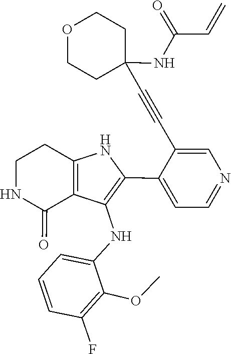 [SMILES: C=CC(=O)NC1(C#Cc2cnccc2-c2[nH]c3c(c2Nc2cccc(F)c2OC)C(=O)NCC3)CCOCC1] |
| 384 | 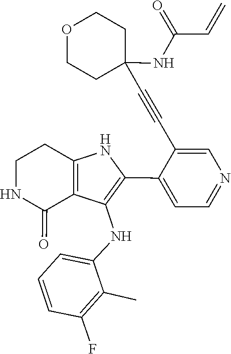 [SMILES: C=CC(=O)NC1(C#Cc2cnccc2-c2[nH]c3c(c2Nc2cccc(F)c2C)C(=O)NCC3)CCOCC1] |
| 385 | 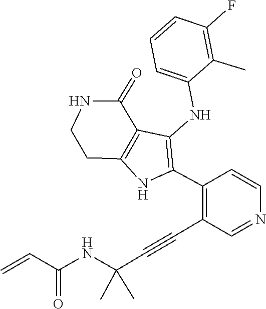 [SMILES: C=CC(=O)NC(C)(C)C#Cc1cnccc1-c1[nH]c2c(c1Nc1cccc(F)c1C)C(=O)NCC2] |
| 386 | 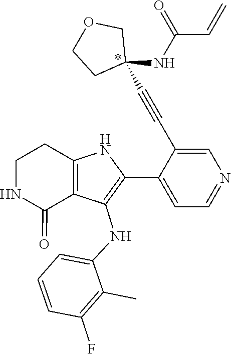 [SMILES: C=CC(=O)N[C@@]1(C#Cc2cnccc2-c2[nH]c3c(c2Nc2cccc(F)c2C)C(=O)NCC3)CCOC1] |
| 387 | 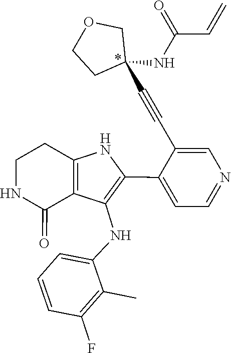 [SMILES: C=CC(=O)N[C@]1(C#Cc2cnccc2-c2[nH]c3c(c2Nc2cccc(F)c2C)C(=O)NCC3)CCOC1] |
| 388 | 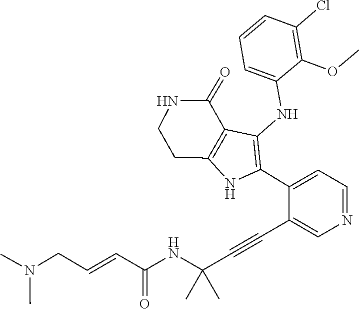 |
| 389 | 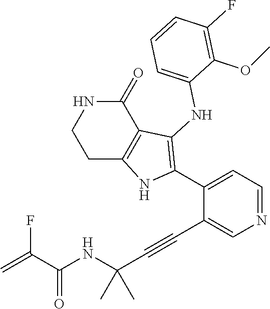 [SMILES: C=C(F)C(=O)NC(C)(C)C#Cc1cnccc1-c1[nH]c2c(c1Nc1cccc(F)c1OC)C(=O)NCC2] |
| 390 | 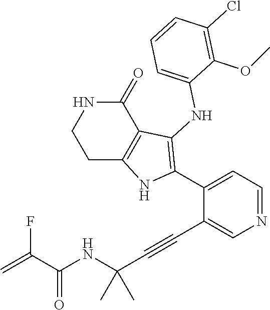 [SMILES: C=C(F)C(=O)NC(C)(C)C#Cc1cnccc1-c1[nH]c2c(c1Nc1cccc(Cl)c1OC)C(=O)NCC2] |
| 391 | 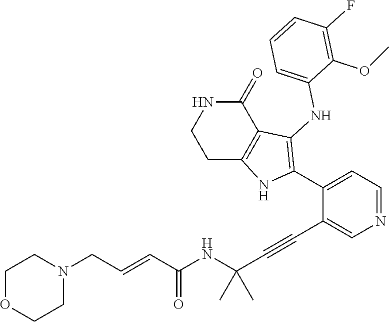 [SMILES: COc1c(F)cccc1Nc1c(-c2ccncc2C#CC(C)(C)NC(=O)/C=C/CN2CCOCC2)[nH]c2c1C(=O)NCC2] |
| 392 | 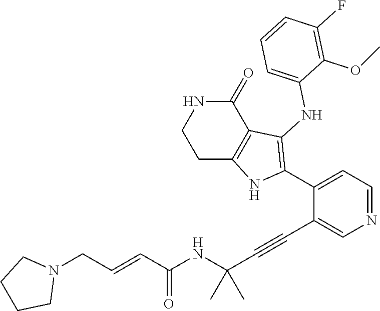 [SMILES: COc1c(F)cccc1Nc1c(-c2ccncc2C#CC(C)(C)NC(=O)/C=C/CN2CCCC2)[nH]c2c1C(=O)NCC2] |
| 393 | 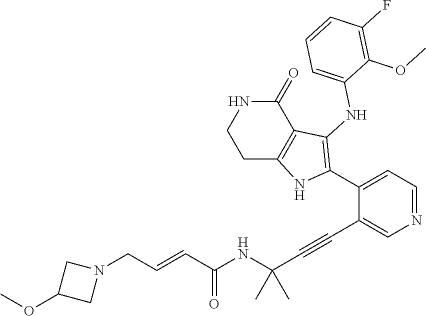 |
| 394 | 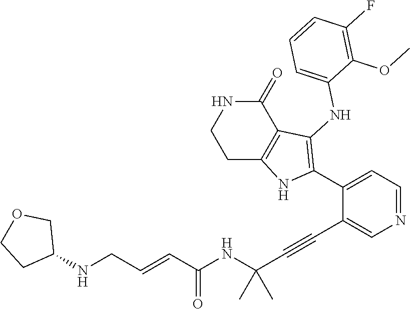 [SMILES: COc1c(F)cccc1Nc1c(-c2ccncc2C#CC(C)(C)NC(=O)/C=C/CN[C@@H]2CCOC2)[nH]c2c1C(=O)NCC2] |
| 395 | 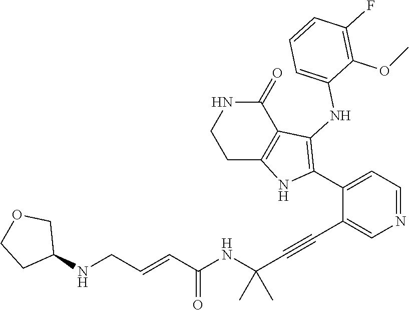 [SMILES: COc1c(F)cccc1Nc1c(-c2ccncc2C#CC(C)(C)NC(=O)/C=C/CN[C@H]2CCOC2)[nH]c2c1C(=O)NCC2] |
| 396 | 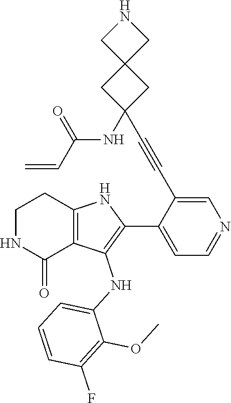 [SMILES: C=CC(=O)NC1(C#Cc2cnccc2-c2[nH]c3c(c2Nc2cccc(F)c2OC)C(=O)NCC3)CC2(CNC2)C1] |
| 397 | 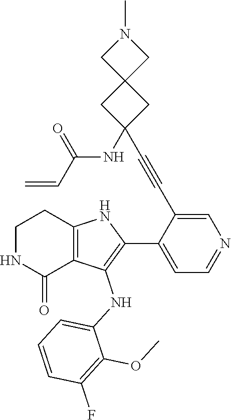 [SMILES: C=CC(=O)NC1(C#Cc2cnccc2-c2[nH]c3c(c2Nc2cccc(F)c2OC)C(=O)NCC3)CC2(CN(C)C2)C1] |
| 398 | 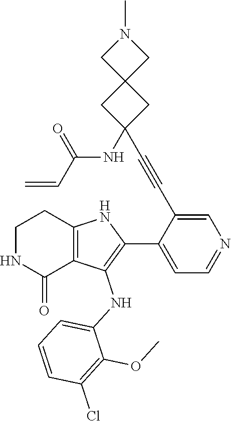 [SMILES: C=CC(=O)NC1(C#Cc2cnccc2-c2[nH]c3c(c2Nc2cccc(Cl)c2OC)C(=O)NCC3)CC2(CN(C)C2)C1] |
| 399 | 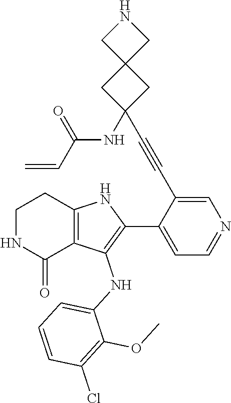 [SMILES: C=CC(=O)NC1(C#Cc2cnccc2-c2[nH]c3c(c2Nc2cccc(Cl)c2OC)C(=O)NCC3)CC2(CNC2)C1] |
| 400 | 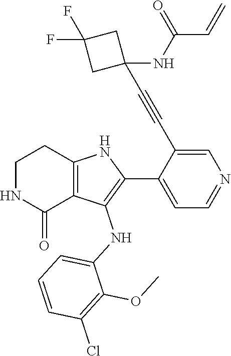 [SMILES: C=CC(=O)NC1(C#Cc2cnccc2-c2[nH]c3c(c2Nc2cccc(Cl)c2OC)C(=O)NCC3)CC(F)(F)C1] |
| 401 | 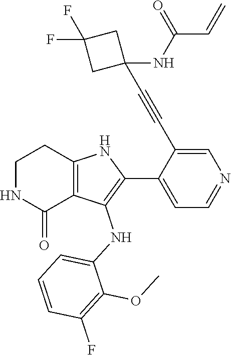 [SMILES: C=CC(=O)NC1(C#Cc2cnccc2-c2[nH]c3c(c2Nc2cccc(F)c2OC)C(=O)NCC3)CC(F)(F)C1] |
| 402 | 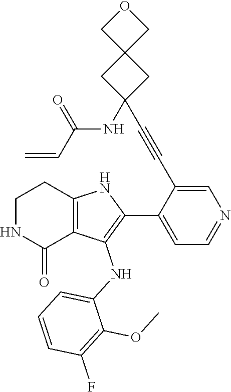 [SMILES: C=CC(=O)NC1(C#Cc2cnccc2-c2[nH]c3c(c2Nc2cccc(F)c2OC)C(=O)NCC3)CC2(COC2)C1] |
| 403 | 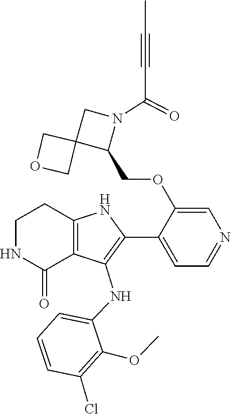 [SMILES: CC#CC(=O)N1CC2(COC2)[C@@H]1COc1cnccc1-c1[nH]c2c(c1Nc1cccc(Cl)c1OC)C(=O)NCC2] |
| 404 | 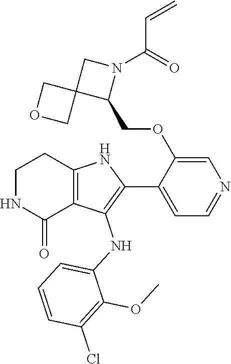 [SMILES: C=CC(=O)N1CC2(COC2)[C@@H]1COc1cnccc1-c1[nH]c2c(c1Nc1cccc(Cl)c1OC)C(=O)NCC2] |
| 405 | 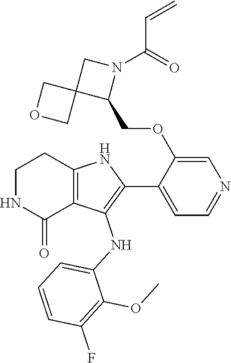 [SMILES: C=CC(=O)N1CC2(COC2)[C@@H]1COc1cnccc1-c1[nH]c2c(c1Nc1cccc(F)c1OC)C(=O)NCC2] |
| 406 | 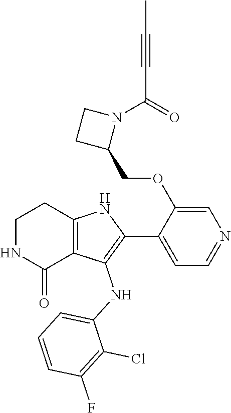 [SMILES: CC#CC(=O)N1CC[C@@H]1COc1cnccc1-c1[nH]c2c(c1Nc1cccc(F)c1Cl)C(=O)NCC2] |
| 407 | 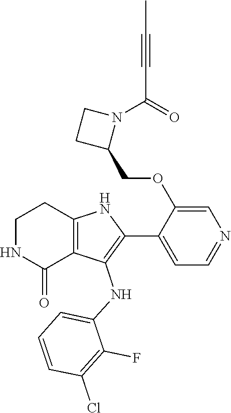 [SMILES: CC#CC(=O)N1CC[C@@H]1COc1cnccc1-c1[nH]c2c(c1Nc1cccc(Cl)c1F)C(=O)NCC2] |
| 408 | 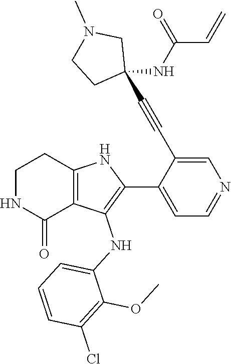 [SMILES: C=CC(=O)N[C@]1(C#Cc2cnccc2-c2[nH]c3c(c2Nc2cccc(Cl)c2OC)C(=O)NCC3)CCN(C)C1] |
| 409 | 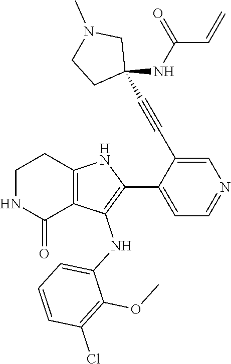 [SMILES: C=CC(=O)N[C@@]1(C#Cc2cnccc2-c2[nH]c3c(c2Nc2cccc(Cl)c2OC)C(=O)NCC3)CCN(C)C1] |
| 410 | 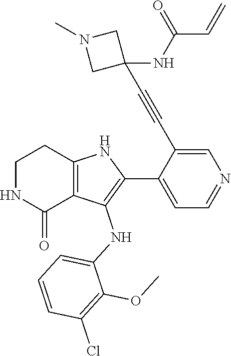 [SMILES: C=CC(=O)NC1(C#Cc2cnccc2-c2[nH]c3c(c2Nc2cccc(Cl)c2OC)C(=O)NCC3)CN(C)C1] |
| 411 | 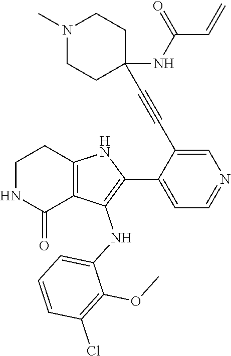 [SMILES: C=CC(=O)NC1(C#Cc2cnccc2-c2[nH]c3c(c2Nc2cccc(Cl)c2OC)C(=O)NCC3)CCN(C)CC1] |
| 412 | 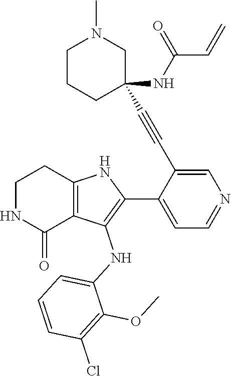 [SMILES: C=CC(=O)N[C@@]1(C#Cc2cnccc2-c2[nH]c3c(c2Nc2cccc(Cl)c2OC)C(=O)NCC3)CCCN(C)C1] |
| 413 | 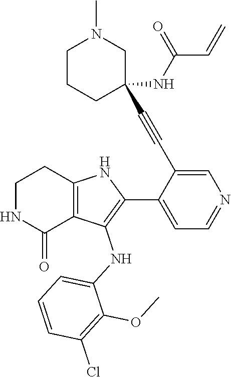 [SMILES: C=CC(=O)N[C@]1(C#Cc2cnccc2-c2[nH]c3c(c2Nc2cccc(Cl)c2OC)C(=O)NCC3)CCCN(C)C1] |
| 414 | 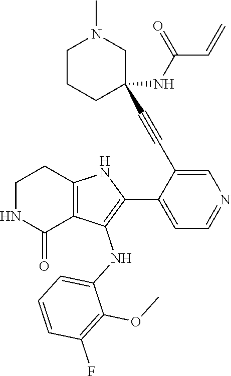 [SMILES: C=CC(=O)N[C@]1(C#Cc2cnccc2-c2[nH]c3c(c2Nc2cccc(F)c2OC)C(=O)NCC3)CCCN(C)C1] |
| 415 | 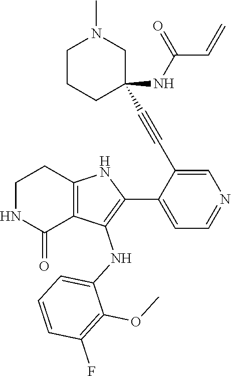 [SMILES: C=CC(=O)N[C@@]1(C#Cc2cnccc2-c2[nH]c3c(c2Nc2cccc(F)c2OC)C(=O)NCC3)CCCN(C)C1] |
| 416 | 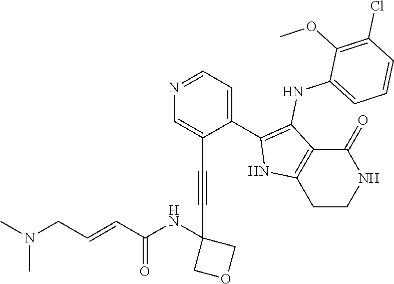 [SMILES: COc1c(Cl)cccc1Nc1c(-c2ccncc2C#CC2(NC(=O)/C=C/CN(C)C)COC2)[nH]c2c1C(=O)NCC2] |
| 417 | 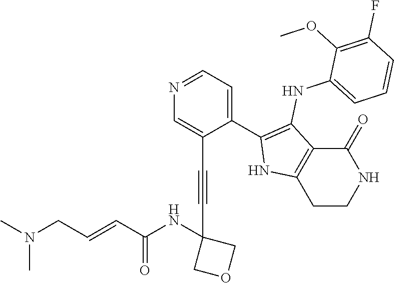 [SMILES: COc1c(F)cccc1Nc1c(-c2ccncc2C#CC2(NC(=O)/C=C/CN(C)C)COC2)[nH]c2c1C(=O)NCC2] |
| 418 | 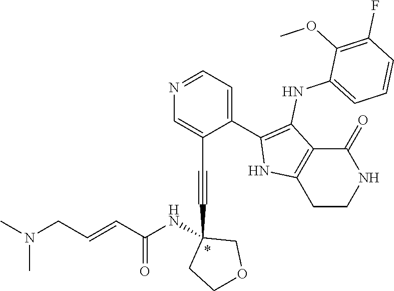 [SMILES: COc1c(F)cccc1Nc1c(-c2ccncc2C#C[C@]2(NC(=O)/C=C/CN(C)C)CCOC2)[nH]c2c1C(=O)NCC2] |
| 419 | 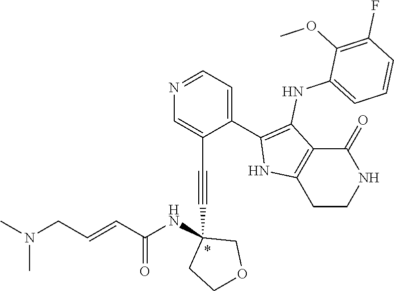 [SMILES: COc1c(F)cccc1Nc1c(-c2ccncc2C#C[C@@]2(NC(=O)/C=C/CN(C)C)CCOC2)[nH]c2c1C(=O)NCC2] |
| 420 | 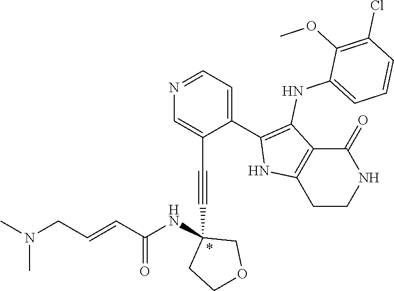 [SMILES: COc1c(Cl)cccc1Nc1c(-c2ccncc2C#C[C@@]2(NC(=O)/C=C/CN(C)C)CCOC2)[nH]c2c1C(=O)NCC2] |
| 421 | 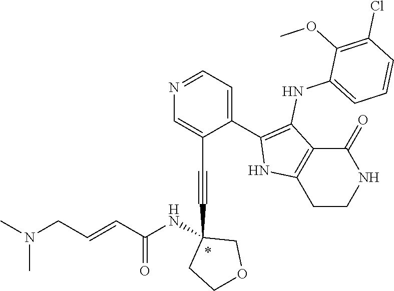 [SMILES: COc1c(Cl)cccc1Nc1c(-c2ccncc2C#C[C@]2(NC(=O)/C=C/CN(C)C)CCOC2)[nH]c2c1C(=O)NCC2] |
| 422 | 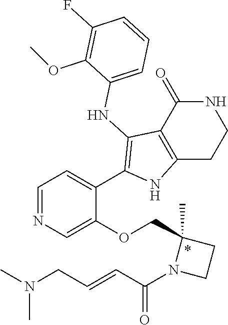 [SMILES: COc1c(F)cccc1Nc1c(-c2ccncc2OC[C@@]2(C)CCN2C(=O)/C=C/CN(C)C)[nH]c2c1C(=O)NCC2] |
| 423 | 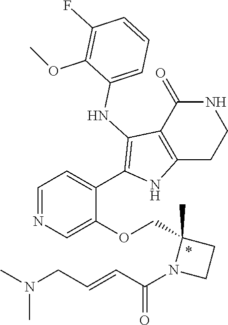 [SMILES: COc1c(F)cccc1Nc1c(-c2ccncc2OC[C@]2(C)CCN2C(=O)/C=C/CN(C)C)[nH]c2c1C(=O)NCC2] |
| 424 | 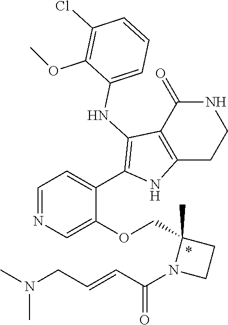 [SMILES: COc1c(Cl)cccc1Nc1c(-c2ccncc2OC[C@]2(C)CCN2C(=O)/C=C/CN(C)C)[nH]c2c1C(=O)NCC2] |
| 425 | 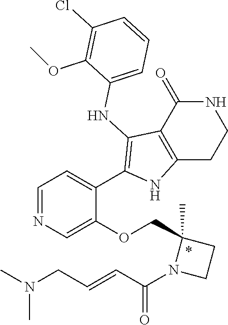 [SMILES: COc1c(Cl)cccc1Nc1c(-c2ccncc2OC[C@@]2(C)CCN2C(=O)/C=C/CN(C)C)[nH]c2c1C(=O)NCC2] |
| 426 | 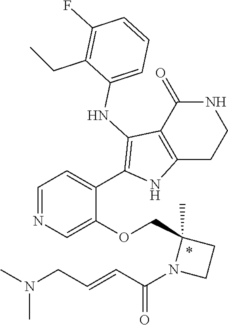 [SMILES: CCc1c(F)cccc1Nc1c(-c2ccncc2OC[C@@]2(C)CCN2C(=O)/C=C/CN(C)C)[nH]c2c1C(=O)NCC2] |
| 427 | 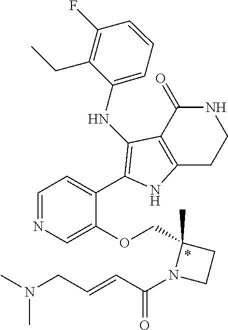 [SMILES: CCc1c(F)cccc1Nc1c(-c2ccncc2OC[C@]2(C)CCN2C(=O)/C=C/CN(C)C)[nH]c2c1C(=O)NCC2] |
| 428 | 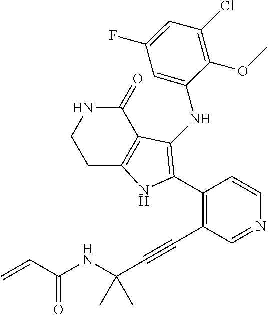 [SMILES: C=CC(=O)NC(C)(C)C#Cc1cnccc1-c1[nH]c2c(c1Nc1cc(F)cc(Cl)c1OC)C(=O)NCC2] |
| 429 | 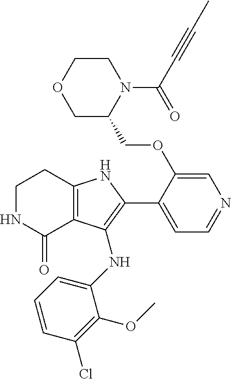 [SMILES: CC#CC(=O)N1CCOC[C@H]1COc1cnccc1-c1[nH]c2c(c1Nc1cccc(Cl)c1OC)C(=O)NCC2] |
| 430 | 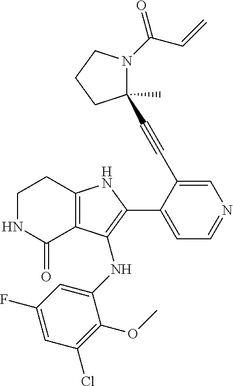 [SMILES: C=CC(=O)N1CCC[C@]1(C)C#Cc1cnccc1-c1[nH]c2c(c1Nc1cc(F)cc(Cl)c1OC)C(=O)NCC2] |
| 431 | 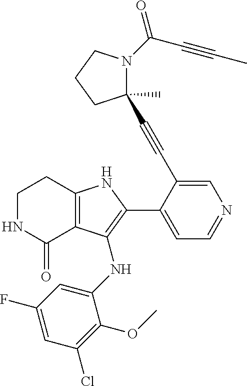 [SMILES: CC#CC(=O)N1CCC[C@]1(C)C#Cc1cnccc1-c1[nH]c2c(c1Nc1cc(F)cc(Cl)c1OC)C(=O)NCC2] |
| 432 | 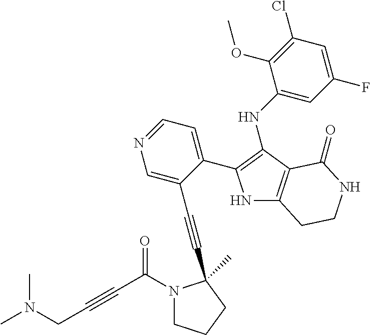 |
| 433 | 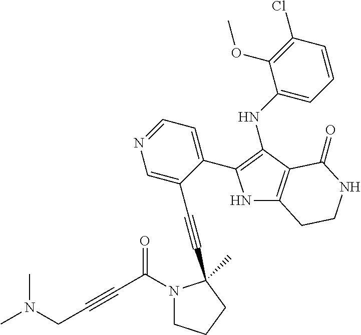 [SMILES: COc1c(Cl)cccc1Nc1c(-c2ccncc2C#C[C@@]2(C)CCCN2C(=O)C#CCN(C)C)[nH]c2c1C(=O)NCC2] |
| 434 | 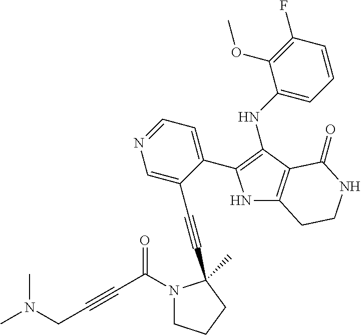 [SMILES: COc1c(F)cccc1Nc1c(-c2ccncc2C#C[C@@]2(C)CCCN2C(=O)C#CCN(C)C)[nH]c2c1C(=O)NCC2] |
| 435 | 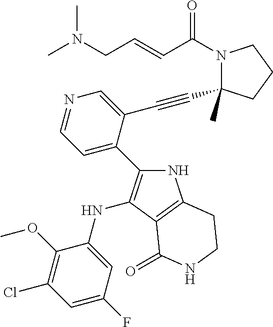 [SMILES: COc1c(Cl)cc(F)cc1Nc1c(-c2ccncc2C#C[C@@]2(C)CCCN2C(=O)/C=C/CN(C)C)[nH]c2c1C(=O)NCC2] |
| 436 | 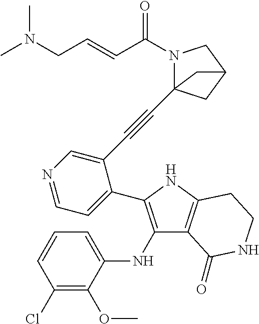 [SMILES: COc1c(Cl)cccc1Nc1c(-c2ccncc2C#CC23CC(CN2C(=O)/C=C/CN(C)C)C3)[nH]c2c1C(=O)NCC2] |
| 437 | 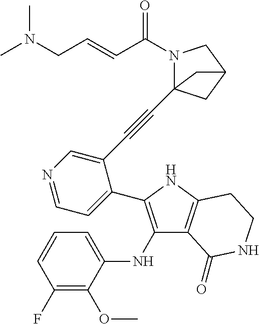 [SMILES: COc1c(F)cccc1Nc1c(-c2ccncc2C#CC23CC(CN2C(=O)/C=C/CN(C)C)C3)[nH]c2c1C(=O)NCC2] |
| 438 | 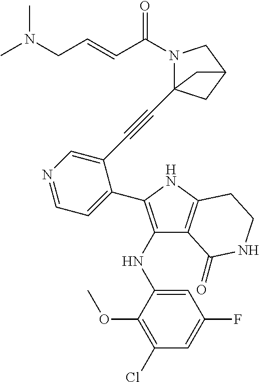 [SMILES: COc1c(Cl)cc(F)cc1Nc1c(-c2ccncc2C#CC23CC(CN2C(=O)/C=C/CN(C)C)C3)[nH]c2c1C(=O)NCC2] |
| 439 | 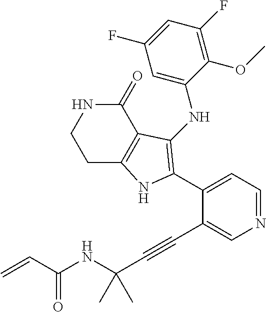 [SMILES: C=CC(=O)NC(C)(C)C#Cc1cnccc1-c1[nH]c2c(c1Nc1cc(F)cc(F)c1OC)C(=O)NCC2] |
| 440 | 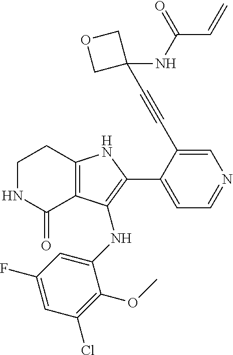 [SMILES: C=CC(=O)NC1(C#Cc2cnccc2-c2[nH]c3c(c2Nc2cc(F)cc(Cl)c2OC)C(=O)NCC3)COC1] |
| 441 | 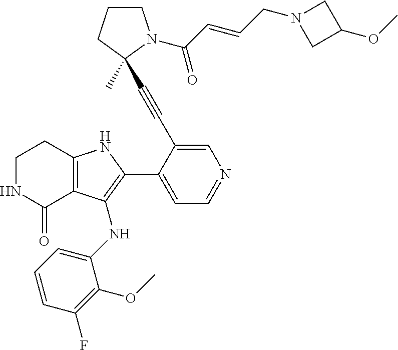 [SMILES: COc1c(F)cccc1Nc1c(-c2ccncc2C#C[C@@]2(C)CCCN2C(=O)/C=C/CN2CC(OC)C2)[nH]c2c1C(=O)NCC2] |
| 442 | 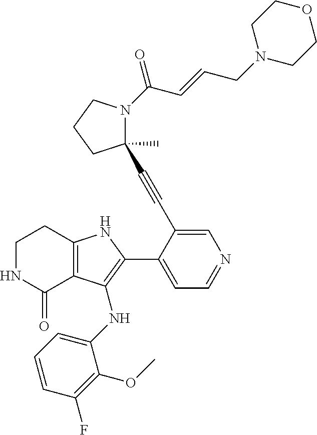 [SMILES: COc1c(F)cccc1Nc1c(-c2ccncc2C#C[C@@]2(C)CCCN2C(=O)/C=C/CN2CCOCC2)[nH]c2c1C(=O)NCC2] |
| 443 | 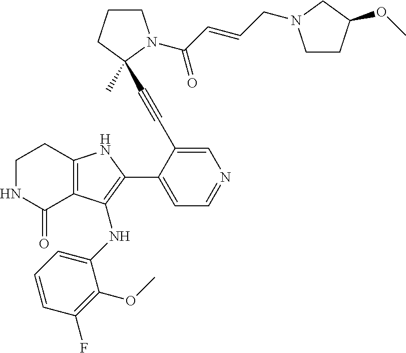 [SMILES: COc1c(F)cccc1Nc1c(-c2ccncc2C#C[C@@]2(C)CCCN2C(=O)/C=C/CN2CC[C@H](OC)C2)[nH]c2c1C(=O)NCC2] |
| 444 | 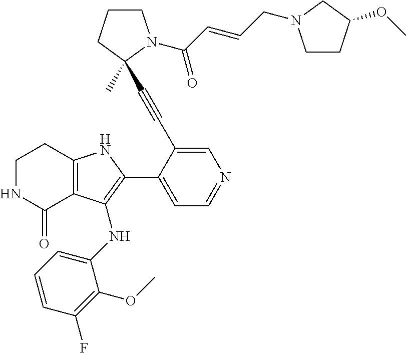 [SMILES: COc1c(F)cccc1Nc1c(-c2ccncc2C#C[C@@]2(C)CCCN2C(=O)/C=C/CN2CC[C@@H](OC)C2)[nH]c2c1C(=O)NCC2] |
| 445 | 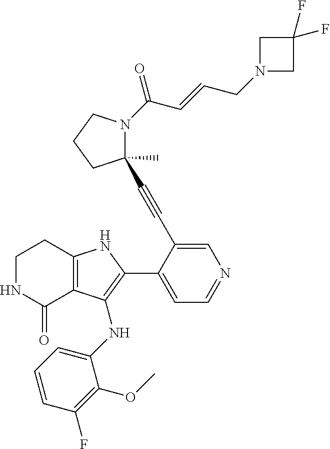 [SMILES: COc1c(F)cccc1Nc1c(-c2ccncc2C#C[C@@]2(C)CCCN2C(=O)/C=C/CN2CC(F)(F)C2)[nH]c2c1C(=O)NCC2] |
| 446 | 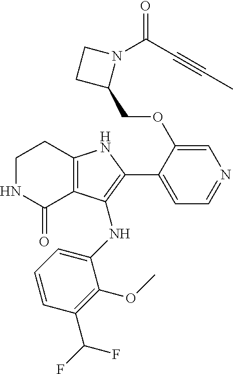 [SMILES: CC#CC(=O)N1CC[C@@H]1COc1cnccc1-c1[nH]c2c(c1Nc1cccc(C(F)F)c1OC)C(=O)NCC2] |
| 447 | 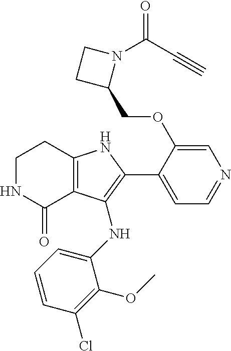 [SMILES: C#CC(=O)N1CC[C@@H]1COc1cnccc1-c1[nH]c2c(c1Nc1cccc(Cl)c1OC)C(=O)NCC2] |
| 448 | 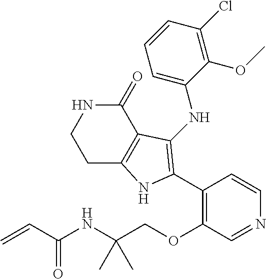 [SMILES: C=CC(=O)NC(C)(C)COc1cnccc1-c1[nH]c2c(c1Nc1cccc(Cl)c1OC)C(=O)NCC2] |
| 449 | 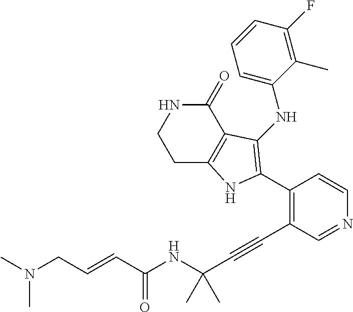 [SMILES: Cc1c(F)cccc1Nc1c(-c2ccncc2C#CC(C)(C)NC(=O)/C=C/CN(C)C)[nH]c2c1C(=O)NCC2] |
| 450 | 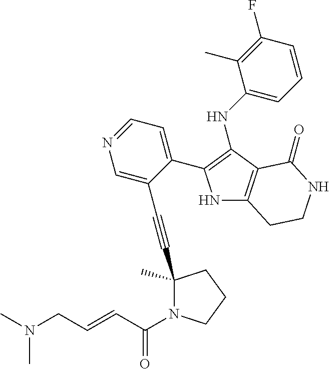 [SMILES: Cc1c(F)cccc1Nc1c(-c2ccncc2C#C[C@@]2(C)CCCN2C(=O)/C=C/CN(C)C)[nH]c2c1C(=O)NCC2] |
| 451 | 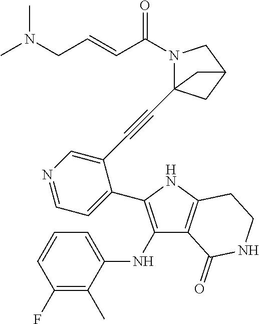 [SMILES: Cc1c(F)cccc1Nc1c(-c2ccncc2C#CC23CC(CN2C(=O)/C=C/CN(C)C)C3)[nH]c2c1C(=O)NCC2] |
| 452 | 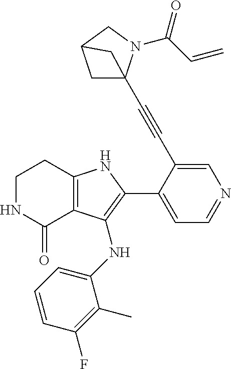 [SMILES: C=CC(=O)N1CC2CC1(C#Cc1cnccc1-c1[nH]c3c(c1Nc1cccc(F)c1C)C(=O)NCC3)C2] |
| 453 | 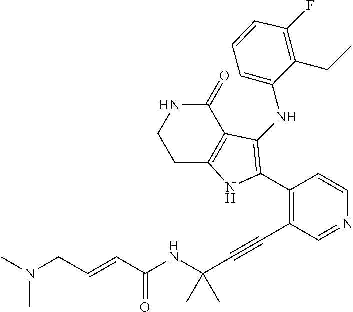 [SMILES: CCc1c(F)cccc1Nc1c(-c2ccncc2C#CC(C)(C)NC(=O)/C=C/CN(C)C)[nH]c2c1C(=O)NCC2] |
| 454 | 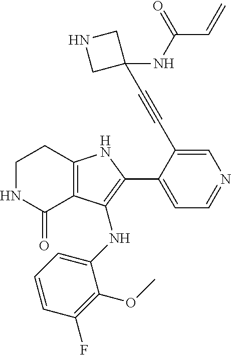 [SMILES: C=CC(=O)NC1(C#Cc2cnccc2-c2[nH]c3c(c2Nc2cccc(F)c2OC)C(=O)NCC3)CNC1] |
| 455 | 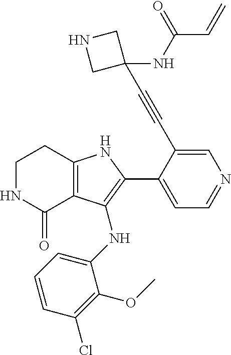 [SMILES: C=CC(=O)NC1(C#Cc2cnccc2-c2[nH]c3c(c2Nc2cccc(Cl)c2OC)C(=O)NCC3)CNC1] |
| 456 | 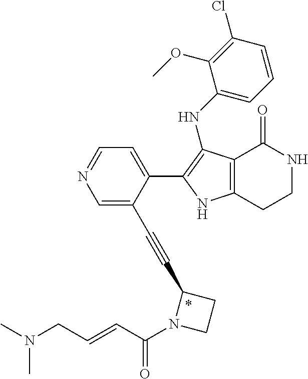 [SMILES: COc1c(Cl)cccc1Nc1c(-c2ccncc2C#C[C@H]2CCN2C(=O)/C=C/CN(C)C)[nH]c2c1C(=O)NCC2] |
| 457 | 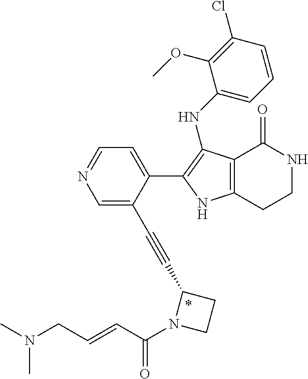 [SMILES: COc1c(Cl)cccc1Nc1c(-c2ccncc2C#C[C@@H]2CCN2C(=O)/C=C/CN(C)C)[nH]c2c1C(=O)NCC2] |
| 458 | 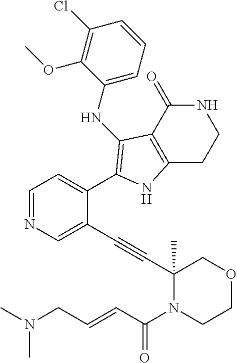 [SMILES: COc1c(Cl)cccc1Nc1c(-c2ccncc2C#C[C@@]2(C)COCCN2C(=O)/C=C/CN(C)C)[nH]c2c1C(=O)NCC2] |
| 459 | 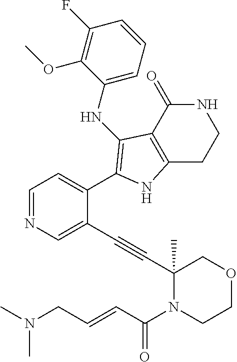 [SMILES: COc1c(F)cccc1Nc1c(-c2ccncc2C#C[C@@]2(C)COCCN2C(=O)/C=C/CN(C)C)[nH]c2c1C(=O)NCC2] |
| 460 | 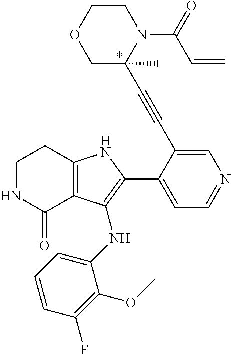 [SMILES: C=CC(=O)N1CCOC[C@]1(C)C#Cc1cnccc1-c1[nH]c2c(c1Nc1cccc(F)c1OC)C(=O)NCC2] |
| 461 | 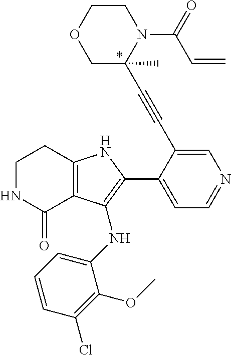 [SMILES: C=CC(=O)N1CCOC[C@]1(C)C#Cc1cnccc1-c1[nH]c2c(c1Nc1cccc(Cl)c1OC)C(=O)NCC2] |
| 462 | 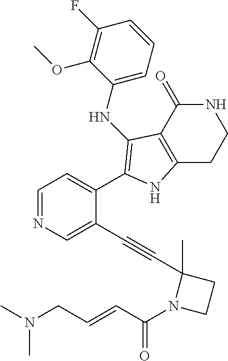 [SMILES: COc1c(F)cccc1Nc1c(-c2ccncc2C#CC2(C)CCN2C(=O)/C=C/CN(C)C)[nH]c2c1C(=O)NCC2] |
| 463 | 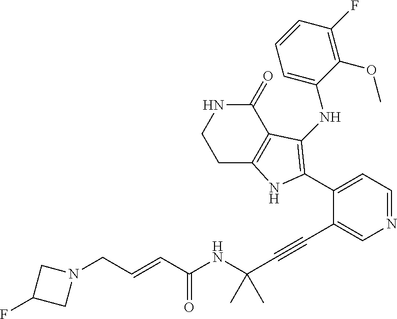 [SMILES: COc1c(F)cccc1Nc1c(-c2ccncc2C#CC(C)(C)NC(=O)/C=C/CN2CC(F)C2)[nH]c2c1C(=O)NCC2] |
| 464 | 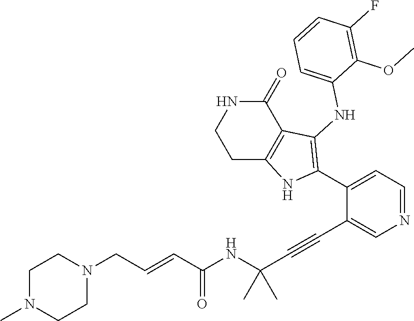 |
| 465 | 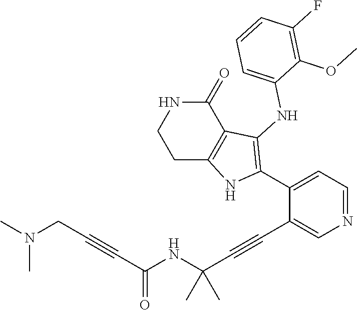 |
| 466 | 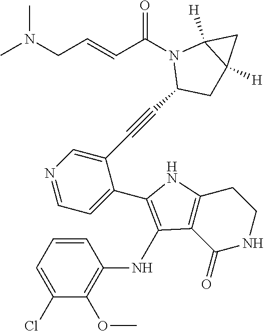 [SMILES: COc1c(Cl)cccc1Nc1c(-c2ccncc2C#C[C@H]2C[C@@H]3C[C@@H]3N2C(=O)/C=C/CN(C)C)[nH]c2c1C(=O)NCC2] |
| 467 | 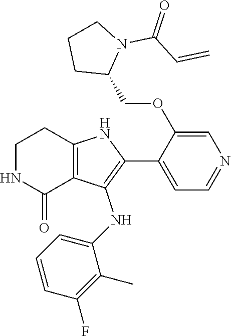 [SMILES: C=CC(=O)N1CCC[C@H]1COc1cnccc1-c1[nH]c2c(c1Nc1cccc(F)c1C)C(=O)NCC2] |
| 468 | 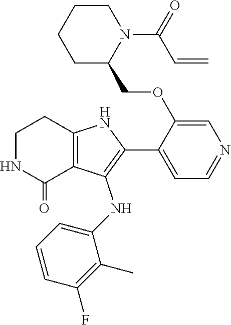 [SMILES: C=CC(=O)N1CCCC[C@@H]1COc1cnccc1-c1[nH]c2c(c1Nc1cccc(F)c1C)C(=O)NCC2] |
| 469 | 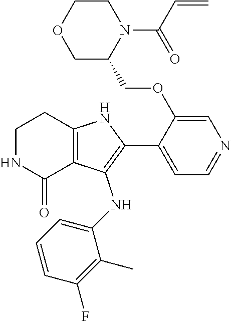 [SMILES: C=CC(=O)N1CCOC[C@H]1COc1cnccc1-c1[nH]c2c(c1Nc1cccc(F)c1C)C(=O)NCC2] |
| 470 | 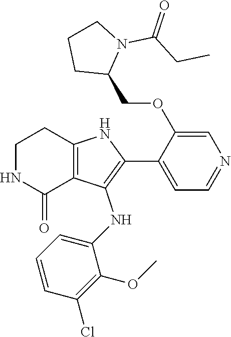 [SMILES: CCC(=O)N1CCC[C@@H]1COc1cnccc1-c1[nH]c2c(c1Nc1cccc(Cl)c1OC)C(=O)NCC2] |
| 471 | 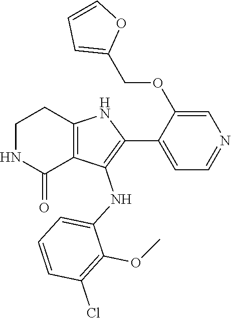 [SMILES: COc1c(Cl)cccc1Nc1c(-c2ccncc2OCc2ccco2)[nH]c2c1C(=O)NCC2] |
| 472 | 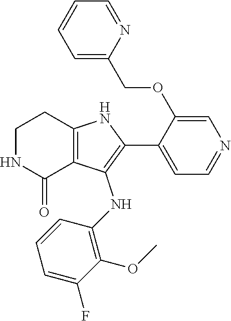 [SMILES: COc1c(F)cccc1Nc1c(-c2ccncc2OCc2ccccn2)[nH]c2c1C(=O)NCC2] |
| 473 | 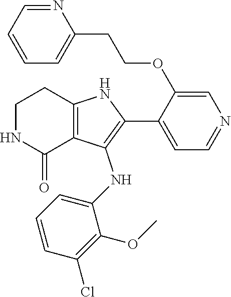 [SMILES: COc1c(Cl)cccc1Nc1c(-c2ccncc2OCCc2ccccn2)[nH]c2c1C(=O)NCC2] |
| 474 | 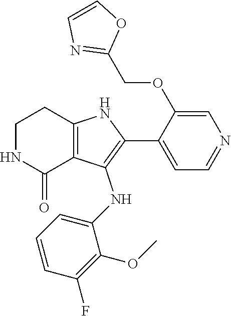 [SMILES: COc1c(F)cccc1Nc1c(-c2ccncc2OCc2ncco2)[nH]c2c1C(=O)NCC2] |
| 475 | 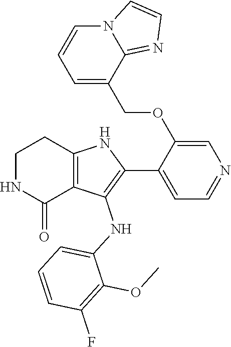 [SMILES: COc1c(F)cccc1Nc1c(-c2ccncc2OCc2cccn3ccnc23)[nH]c2c1C(=O)NCC2] |
| 476 | 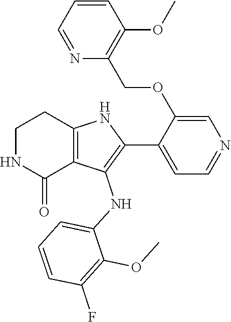 [SMILES: COc1cccnc1COc1cnccc1-c1[nH]c2c(c1Nc1cccc(F)c1OC)C(=O)NCC2] |
| 477 | 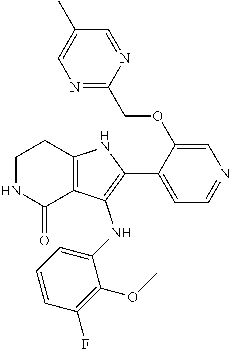 [SMILES: COc1c(F)cccc1Nc1c(-c2ccncc2OCc2ncc(C)cn2)[nH]c2c1C(=O)NCC2] |
| 478 | 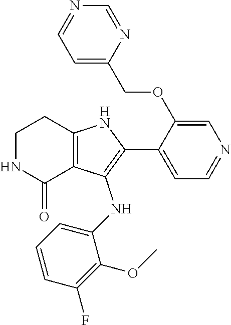 [SMILES: COc1c(F)cccc1Nc1c(-c2ccncc2OCc2ccncn2)[nH]c2c1C(=O)NCC2] |
| 479 | 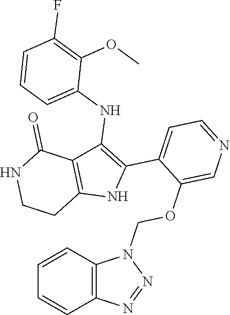 [SMILES: COc1c(F)cccc1Nc1c(-c2ccncc2OCn2nnc3ccccc32)[nH]c2c1C(=O)NCC2] |
| 480 | 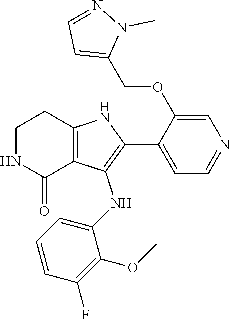 [SMILES: COc1c(F)cccc1Nc1c(-c2ccncc2OCc2ccnn2C)[nH]c2c1C(=O)NCC2] |
| 481 | 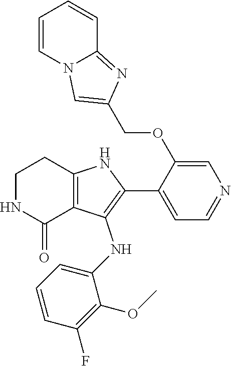 [SMILES: COc1c(F)cccc1Nc1c(-c2ccncc2OCc2cn3ccccc3n2)[nH]c2c1C(=O)NCC2] |
| 482 | 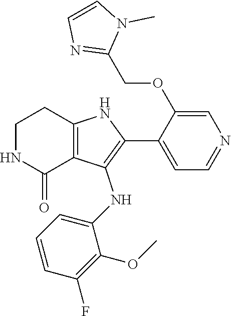 [SMILES: COc1c(F)cccc1Nc1c(-c2ccncc2OCc2nccn2C)[nH]c2c1C(=O)NCC2] |
| 483 | 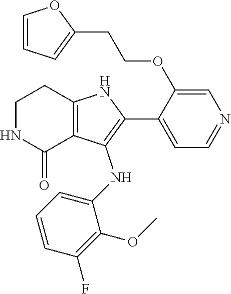 [SMILES: COc1c(F)cccc1Nc1c(-c2ccncc2OCCc2ccco2)[nH]c2c1C(=O)NCC2] |
| 484 | 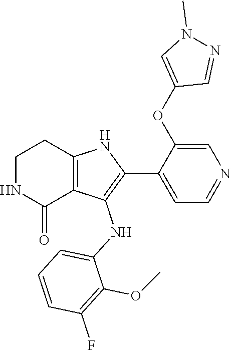 [SMILES: COc1c(F)cccc1Nc1c(-c2ccncc2Oc2cnn(C)c2)[nH]c2c1C(=O)NCC2] |
| 485 | 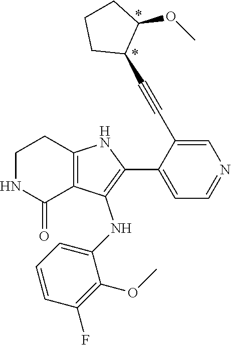 [SMILES: COc1c(F)cccc1Nc1c(-c2ccncc2C#C[C@H]2CCC[C@H]2OC)[nH]c2c1C(=O)NCC2] |
| 486 | 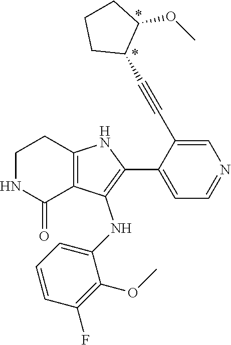 [SMILES: COc1c(F)cccc1Nc1c(-c2ccncc2C#C[C@@H]2CCC[C@@H]2OC)[nH]c2c1C(=O)NCC2] |
| 487 | 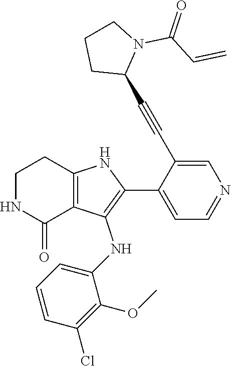 [SMILES: C=CC(=O)N1CCC[C@@H]1C#Cc1cnccc1-c1[nH]c2c(c1Nc1cccc(Cl)c1OC)C(=O)NCC2] |
| 488 | 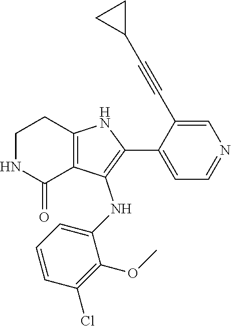 [SMILES: COc1c(Cl)cccc1Nc1c(-c2ccncc2C#CC2CC2)[nH]c2c1C(=O)NCC2] |
| 489 | 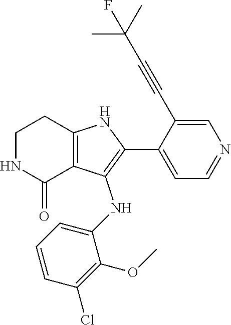 [SMILES: COc1c(Cl)cccc1Nc1c(-c2ccncc2C#CC(C)(C)F)[nH]c2c1C(=O)NCC2] |
| 490 | 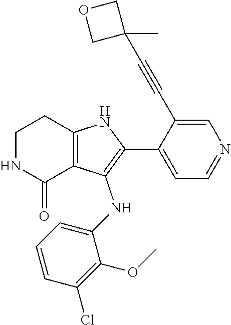 [SMILES: COc1c(Cl)cccc1Nc1c(-c2ccncc2C#CC2(C)COC2)[nH]c2c1C(=O)NCC2] |
| 491 | 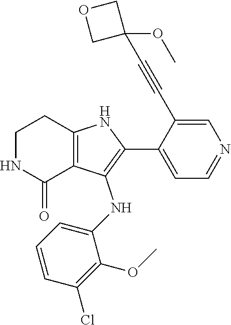 [SMILES: COc1c(Cl)cccc1Nc1c(-c2ccncc2C#CC2(OC)COC2)[nH]c2c1C(=O)NCC2] |
| 492 | 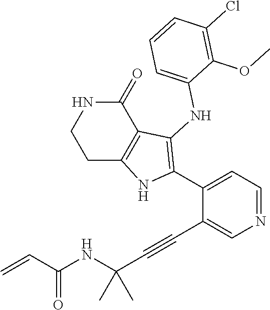 [SMILES: C=CC(=O)NC(C)(C)C#Cc1cnccc1-c1[nH]c2c(c1Nc1cccc(Cl)c1OC)C(=O)NCC2] |
| 493 | 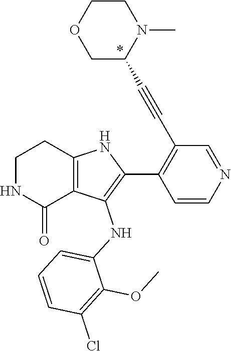 [SMILES: COc1c(Cl)cccc1Nc1c(-c2ccncc2C#C[C@@H]2COCCN2C)[nH]c2c1C(=O)NCC2] |
| 494 | 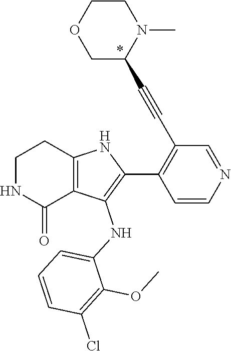 [SMILES: COc1c(Cl)cccc1Nc1c(-c2ccncc2C#C[C@H]2COCCN2C)[nH]c2c1C(=O)NCC2] |
| 495 | 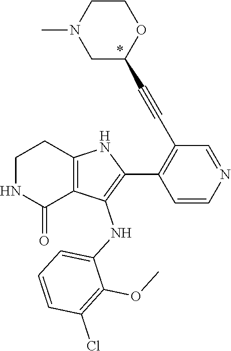 [SMILES: COc1c(Cl)cccc1Nc1c(-c2ccncc2C#C[C@H]2CN(C)CCO2)[nH]c2c1C(=O)NCC2] |
| 496 | 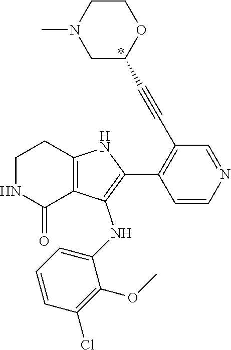 [SMILES: COc1c(Cl)cccc1Nc1c(-c2ccncc2C#C[C@@H]2CN(C)CCO2)[nH]c2c1C(=O)NCC2] |
| 497 | 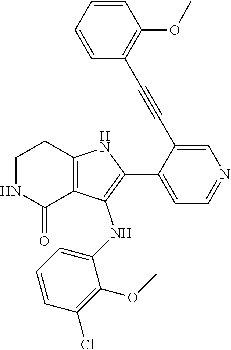 [SMILES: COc1ccccc1C#Cc1cnccc1-c1[nH]c2c(c1Nc1cccc(Cl)c1OC)C(=O)NCC2] |
| 498 | 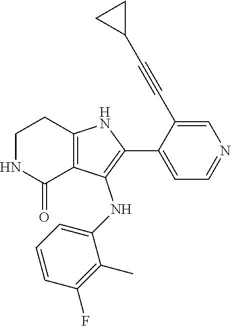 [SMILES: Cc1c(F)cccc1Nc1c(-c2ccncc2C#CC2CC2)[nH]c2c1C(=O)NCC2] |
| 499 | 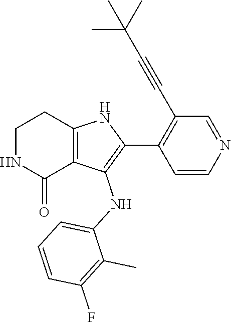 [SMILES: Cc1c(F)cccc1Nc1c(-c2ccncc2C#CC(C)(C)C)[nH]c2c1C(=O)NCC2] |
| 500 | 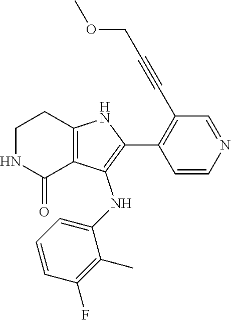 [SMILES: COCC#Cc1cnccc1-c1[nH]c2c(c1Nc1cccc(F)c1C)C(=O)NCC2] |
| 501 | 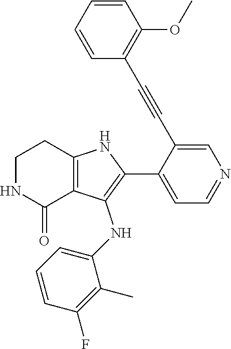 [SMILES: COc1ccccc1C#Cc1cnccc1-c1[nH]c2c(c1Nc1cccc(F)c1C)C(=O)NCC2] |
| 502 | 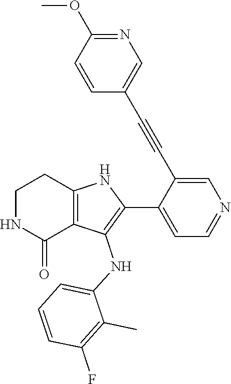 [SMILES: COc1ccc(C#Cc2cnccc2-c2[nH]c3c(c2Nc2cccc(F)c2C)C(=O)NCC3)cn1] |
| 503 | 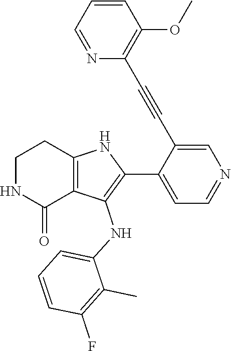 [SMILES: COc1cccnc1C#Cc1cnccc1-c1[nH]c2c(c1Nc1cccc(F)c1C)C(=O)NCC2] |
| 504 | 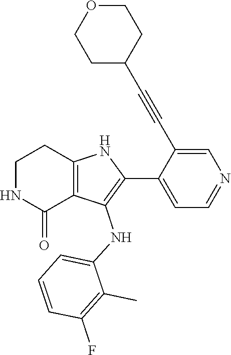 [SMILES: Cc1c(F)cccc1Nc1c(-c2ccncc2C#CC2CCOCC2)[nH]c2c1C(=O)NCC2] |
| 505 | 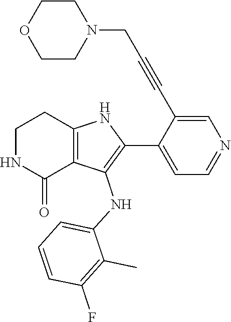 [SMILES: Cc1c(F)cccc1Nc1c(-c2ccncc2C#CCN2CCOCC2)[nH]c2c1C(=O)NCC2] |
| 506 | 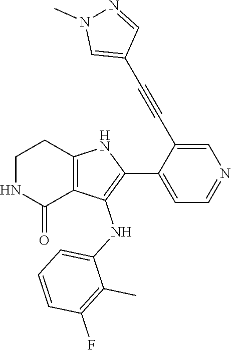 [SMILES: Cc1c(F)cccc1Nc1c(-c2ccncc2C#Cc2cnn(C)c2)[nH]c2c1C(=O)NCC2] |
| 507 | 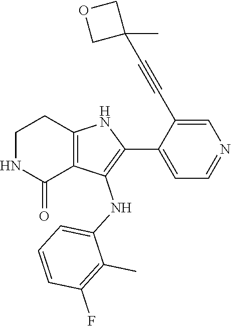 [SMILES: Cc1c(F)cccc1Nc1c(-c2ccncc2C#CC2(C)COC2)[nH]c2c1C(=O)NCC2] |
| 508 | 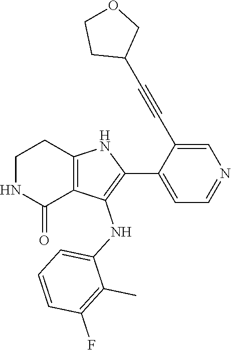 [SMILES: Cc1c(F)cccc1Nc1c(-c2ccncc2C#CC2CCOC2)[nH]c2c1C(=O)NCC2] |
| 509 | 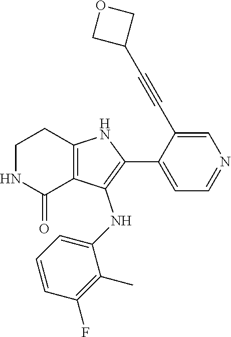 [SMILES: Cc1c(F)cccc1Nc1c(-c2ccncc2C#CC2COC2)[nH]c2c1C(=O)NCC2] |
| 510 | 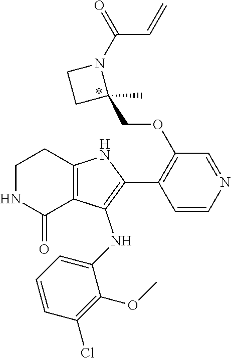 [SMILES: C=CC(=O)N1CC[C@]1(C)COc1cnccc1-c1[nH]c2c(c1Nc1cccc(Cl)c1OC)C(=O)NCC2] |
| 511 | 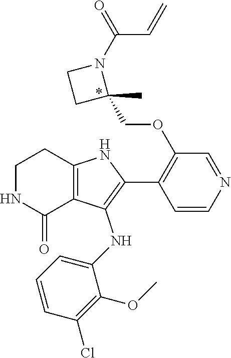 [SMILES: C=CC(=O)N1CC[C@@]1(C)COc1cnccc1-c1[nH]c2c(c1Nc1cccc(Cl)c1OC)C(=O)NCC2] |
| 512 | 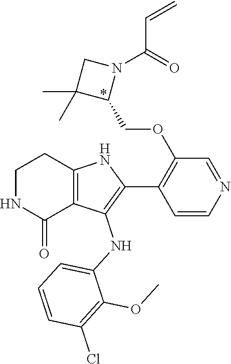 [SMILES: C=CC(=O)N1CC(C)(C)[C@H]1COc1cnccc1-c1[nH]c2c(c1Nc1cccc(Cl)c1OC)C(=O)NCC2] |
| 513 | 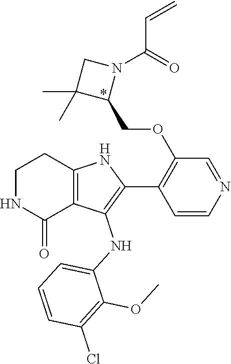 [SMILES: C=CC(=O)N1CC(C)(C)[C@@H]1COc1cnccc1-c1[nH]c2c(c1Nc1cccc(Cl)c1OC)C(=O)NCC2] |
| 514 | 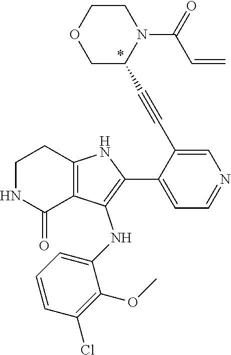 [SMILES: C=CC(=O)N1CCOC[C@H]1C#Cc1cnccc1-c1[nH]c2c(c1Nc1cccc(Cl)c1OC)C(=O)NCC2] |
| 515 | 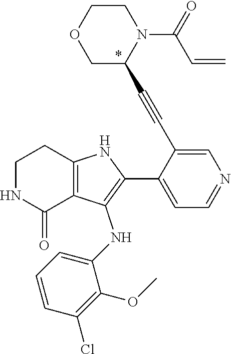 [SMILES: C=CC(=O)N1CCOC[C@@H]1C#Cc1cnccc1-c1[nH]c2c(c1Nc1cccc(Cl)c1OC)C(=O)NCC2] |
| 516 | 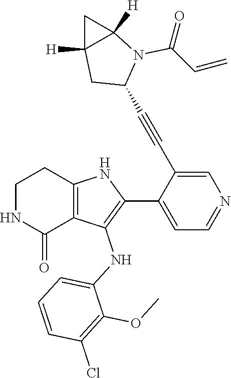 [SMILES: C=CC(=O)N1[C@H](C#Cc2cnccc2-c2[nH]c3c(c2Nc2cccc(Cl)c2OC)C(=O)NCC3)C[C@@H]2C[C@@H]21] |
| 517 | 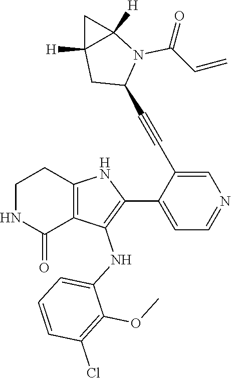 [SMILES: C=CC(=O)N1[C@@H](C#Cc2cnccc2-c2[nH]c3c(c2Nc2cccc(Cl)c2OC)C(=O)NCC3)C[C@@H]2C[C@@H]21] |
| 518 | 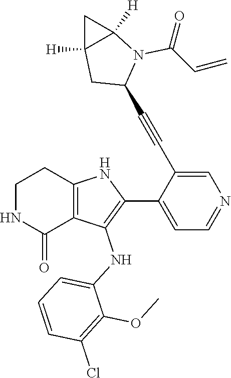 [SMILES: C=CC(=O)N1[C@@H]2C[C@@H]2C[C@@H]1C#Cc1cnccc1-c1[nH]c2c(c1Nc1cccc(Cl)c1OC)C(=O)NCC2] |
| 519 | 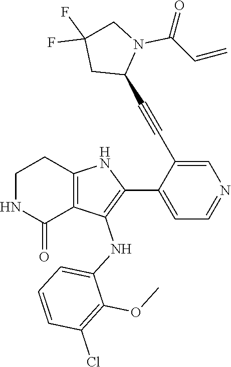 [SMILES: C=CC(=O)N1CC(F)(F)C[C@@H]1C#Cc1cnccc1-c1[nH]c2c(c1Nc1cccc(Cl)c1OC)C(=O)NCC2] |
| 520 | 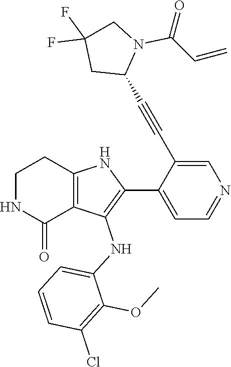 [SMILES: C=CC(=O)N1CC(F)(F)C[C@H]1C#Cc1cnccc1-c1[nH]c2c(c1Nc1cccc(Cl)c1OC)C(=O)NCC2] |
| 521 | 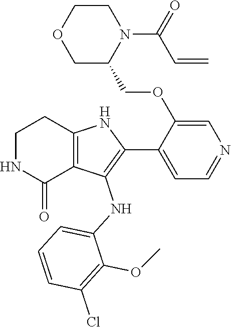 [SMILES: C=CC(=O)N1CCOC[C@H]1COc1cnccc1-c1[nH]c2c(c1Nc1cccc(Cl)c1OC)C(=O)NCC2] |
| 522 | 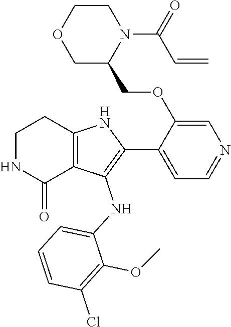 [SMILES: C=CC(=O)N1CCOC[C@@H]1COc1cnccc1-c1[nH]c2c(c1Nc1cccc(Cl)c1OC)C(=O)NCC2] |
| 523 | 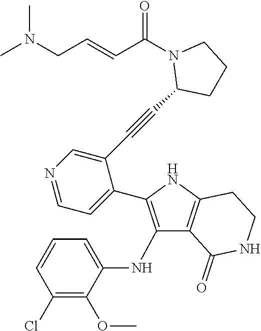 [SMILES: COc1c(Cl)cccc1Nc1c(-c2ccncc2C#C[C@H]2CCCN2C(=O)/C=C/CN(C)C)[nH]c2c1C(=O)NCC2] |
| 524 | 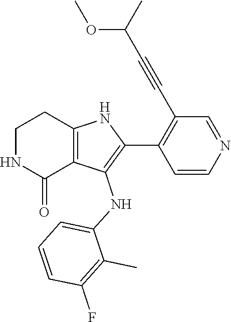 [SMILES: COC(C)C#Cc1cnccc1-c1[nH]c2c(c1Nc1cccc(F)c1C)C(=O)NCC2] |
| 525 | 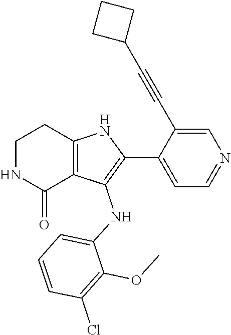 [SMILES: COc1c(Cl)cccc1Nc1c(-c2ccncc2C#CC2CCC2)[nH]c2c1C(=O)NCC2] |
| 526 | 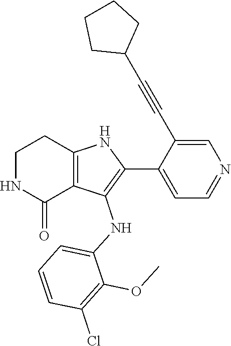 [SMILES: COc1c(Cl)cccc1Nc1c(-c2ccncc2C#CC2CCCC2)[nH]c2c1C(=O)NCC2] |
| 527 | 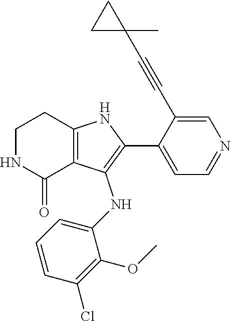 [SMILES: COc1c(Cl)cccc1Nc1c(-c2ccncc2C#CC2(C)CC2)[nH]c2c1C(=O)NCC2] |
| 528 | 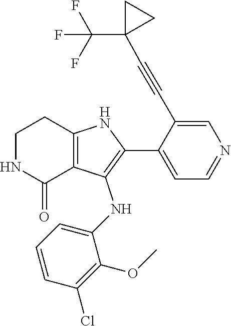 [SMILES: COc1c(Cl)cccc1Nc1c(-c2ccncc2C#CC2(C(F)(F)F)CC2)[nH]c2c1C(=O)NCC2] |
| 529 | 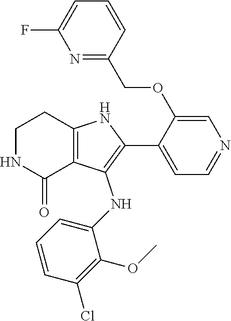 [SMILES: COc1c(Cl)cccc1Nc1c(-c2ccncc2OCc2cccc(F)n2)[nH]c2c1C(=O)NCC2] |
| 530 | 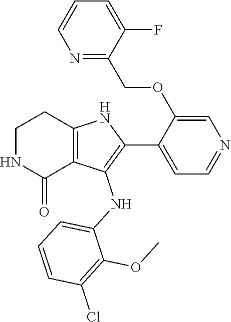 [SMILES: COc1c(Cl)cccc1Nc1c(-c2ccncc2OCc2ncccc2F)[nH]c2c1C(=O)NCC2] |
| 531 | 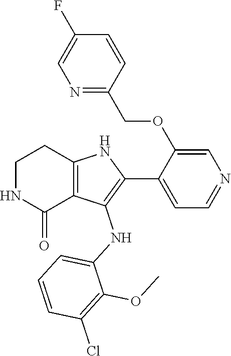 [SMILES: COc1c(Cl)cccc1Nc1c(-c2ccncc2OCc2ccc(F)cn2)[nH]c2c1C(=O)NCC2] |
| 532 | 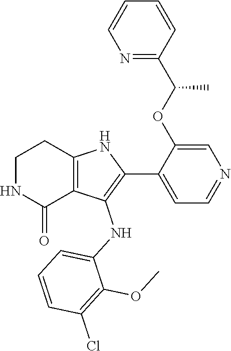 [SMILES: COc1c(Cl)cccc1Nc1c(-c2ccncc2O[C@@H](C)c2ccccn2)[nH]c2c1C(=O)NCC2] |
| 533 | 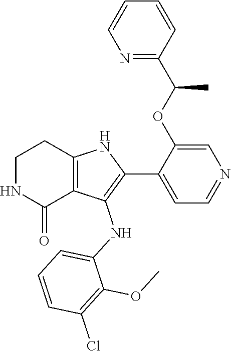 [SMILES: COc1c(Cl)cccc1Nc1c(-c2ccncc2O[C@H](C)c2ccccn2)[nH]c2c1C(=O)NCC2] |
| 534 | 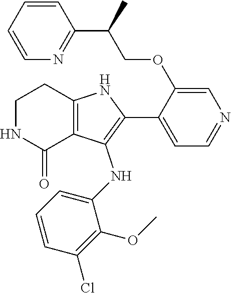 [SMILES: COc1c(Cl)cccc1Nc1c(-c2ccncc2OC[C@H](C)c2ccccn2)[nH]c2c1C(=O)NCC2] |
| 535 | 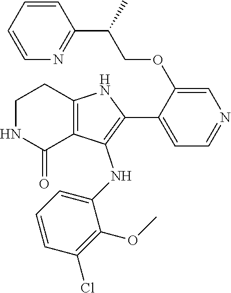 [SMILES: COc1c(Cl)cccc1Nc1c(-c2ccncc2OC[C@@H](C)c2ccccn2)[nH]c2c1C(=O)NCC2] |
| 536 | 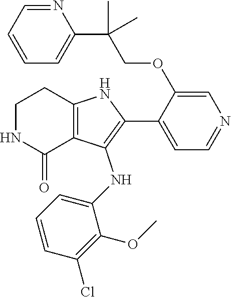 [SMILES: COc1c(Cl)cccc1Nc1c(-c2ccncc2OCC(C)(C)c2ccccn2)[nH]c2c1C(=O)NCC2] |
| 537 | 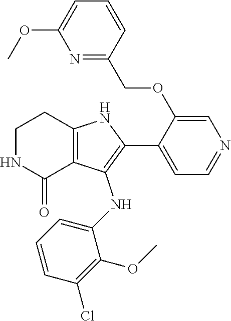 [SMILES: COc1cccc(COc2cnccc2-c2[nH]c3c(c2Nc2cccc(Cl)c2OC)C(=O)NCC3)n1] |
| 538 | 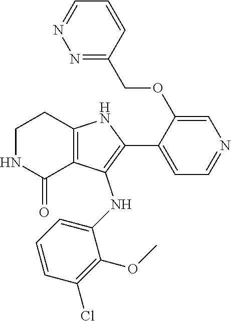 [SMILES: COc1c(Cl)cccc1Nc1c(-c2ccncc2OCc2cccnn2)[nH]c2c1C(=O)NCC2] |
| 539 | 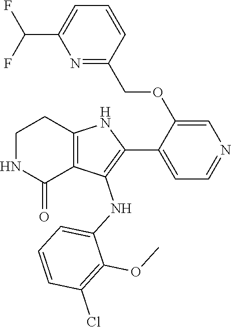 [SMILES: COc1c(Cl)cccc1Nc1c(-c2ccncc2OCc2cccc(C(F)F)n2)[nH]c2c1C(=O)NCC2] |
| 540 | 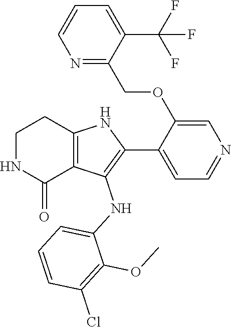 [SMILES: COc1c(Cl)cccc1Nc1c(-c2ccncc2OCc2ncccc2C(F)(F)F)[nH]c2c1C(=O)NCC2] |
| 541 | 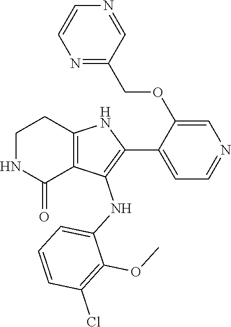 [SMILES: COc1c(Cl)cccc1Nc1c(-c2ccncc2OCc2cnccn2)[nH]c2c1C(=O)NCC2] |
| 542 | 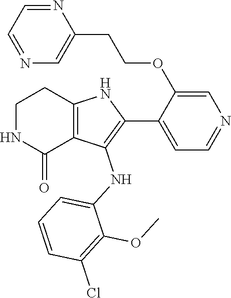 [SMILES: COc1c(Cl)cccc1Nc1c(-c2ccncc2OCCc2cnccn2)[nH]c2c1C(=O)NCC2] |
| 543 | 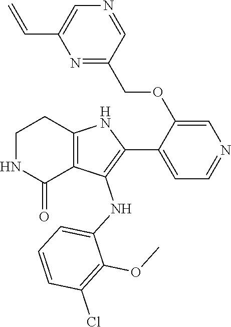 [SMILES: C=Cc1cncc(COc2cnccc2-c2[nH]c3c(c2Nc2cccc(Cl)c2OC)C(=O)NCC3)n1] |
| 544 | 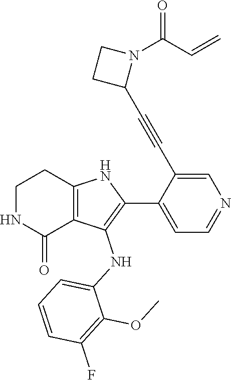 [SMILES: C=CC(=O)N1CCC1C#Cc1cnccc1-c1[nH]c2c(c1Nc1cccc(F)c1OC)C(=O)NCC2] |
| 545 | 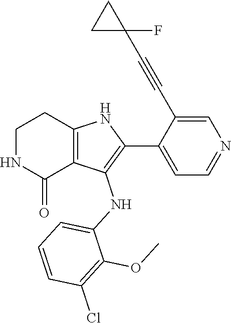 [SMILES: COc1c(Cl)cccc1Nc1c(-c2ccncc2C#CC2(F)CC2)[nH]c2c1C(=O)NCC2] |
| 546 | 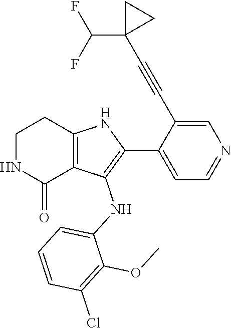 [SMILES: COc1c(Cl)cccc1Nc1c(-c2ccncc2C#CC2(C(F)F)CC2)[nH]c2c1C(=O)NCC2] |
| 547 | 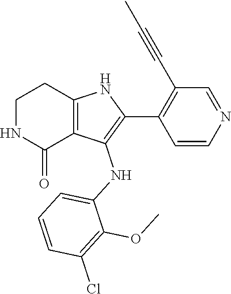 [SMILES: CC#Cc1cnccc1-c1[nH]c2c(c1Nc1cccc(Cl)c1OC)C(=O)NCC2] |
| 548 | 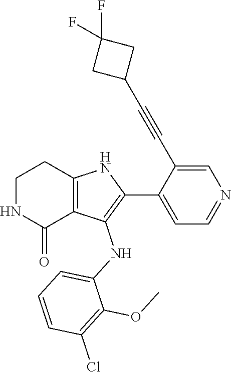 [SMILES: COc1c(Cl)cccc1Nc1c(-c2ccncc2C#CC2CC(F)(F)C2)[nH]c2c1C(=O)NCC2] |
| 549 | 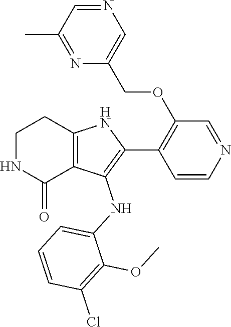 [SMILES: COc1c(Cl)cccc1Nc1c(-c2ccncc2OCc2cncc(C)n2)[nH]c2c1C(=O)NCC2] |
| 550 | 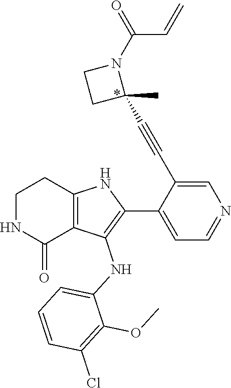 [SMILES: C=CC(=O)N1CC[C@@]1(C)C#Cc1cnccc1-c1[nH]c2c(c1Nc1cccc(Cl)c1OC)C(=O)NCC2] |
| 551 | 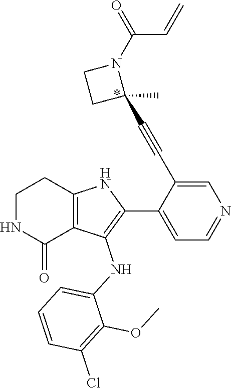 [SMILES: C=CC(=O)N1CC[C@]1(C)C#Cc1cnccc1-c1[nH]c2c(c1Nc1cccc(Cl)c1OC)C(=O)NCC2] |
| 552 | 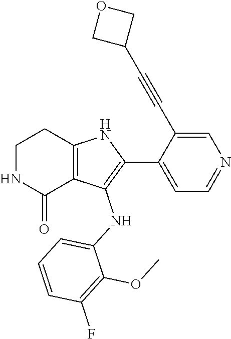 [SMILES: COc1c(F)cccc1Nc1c(-c2ccncc2C#CC2COC2)[nH]c2c1C(=O)NCC2] |
| 553 | 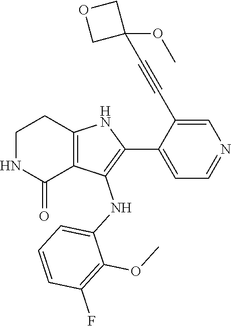 [SMILES: COc1c(F)cccc1Nc1c(-c2ccncc2C#CC2(OC)COC2)[nH]c2c1C(=O)NCC2] |
| 554 | 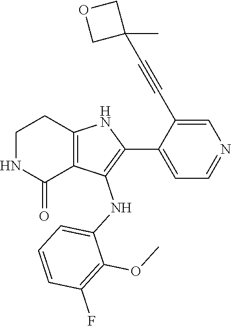 [SMILES: COc1c(F)cccc1Nc1c(-c2ccncc2C#CC2(C)COC2)[nH]c2c1C(=O)NCC2] |
| 555 | 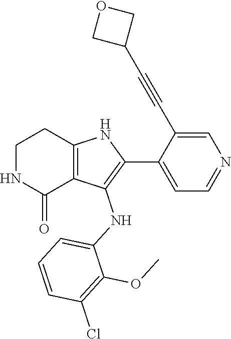 [SMILES: COc1c(Cl)cccc1Nc1c(-c2ccncc2C#CC2COC2)[nH]c2c1C(=O)NCC2] |
| 556 | 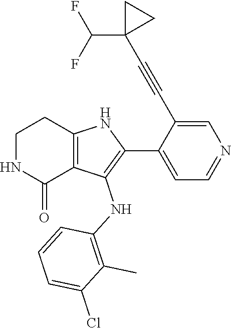 [SMILES: Cc1c(Cl)cccc1Nc1c(-c2ccncc2C#CC2(C(F)F)CC2)[nH]c2c1C(=O)NCC2] |
| 557 | 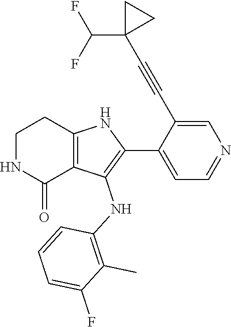 [SMILES: Cc1c(F)cccc1Nc1c(-c2ccncc2C#CC2(C(F)F)CC2)[nH]c2c1C(=O)NCC2] |
| 558 | 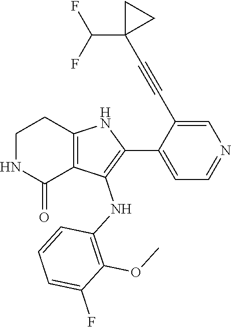 [SMILES: COc1c(F)cccc1Nc1c(-c2ccncc2C#CC2(C(F)F)CC2)[nH]c2c1C(=O)NCC2] |
| 559 | 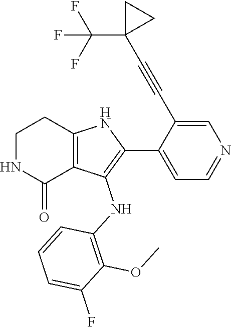 [SMILES: COc1c(F)cccc1Nc1c(-c2ccncc2C#CC2(C(F)(F)F)CC2)[nH]c2c1C(=O)NCC2] |
| 560 | 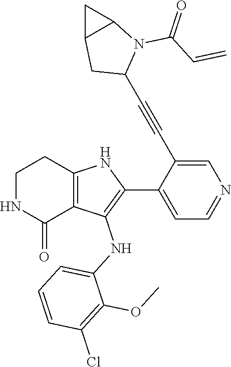 [SMILES: C=CC(=O)N1C(C#Cc2cnccc2-c2[nH]c3c(c2Nc2cccc(Cl)c2OC)C(=O)NCC3)CC2CC21] |
| 561 | 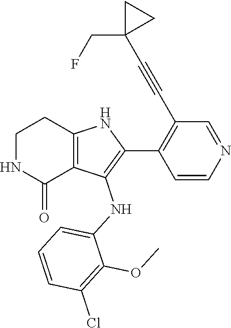 [SMILES: COc1c(Cl)cccc1Nc1c(-c2ccncc2C#CC2(CF)CC2)[nH]c2c1C(=O)NCC2] |
| 562 | 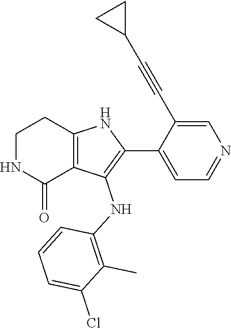 [SMILES: Cc1c(Cl)cccc1Nc1c(-c2ccncc2C#CC2CC2)[nH]c2c1C(=O)NCC2] |
| 563 | 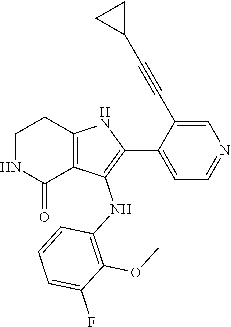 [SMILES: COc1c(F)cccc1Nc1c(-c2ccncc2C#CC2CC2)[nH]c2c1C(=O)NCC2] |
| 564 | 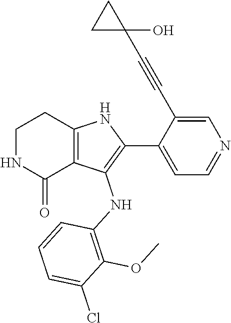 [SMILES: COc1c(Cl)cccc1Nc1c(-c2ccncc2C#CC2(O)CC2)[nH]c2c1C(=O)NCC2] |
| 565 | 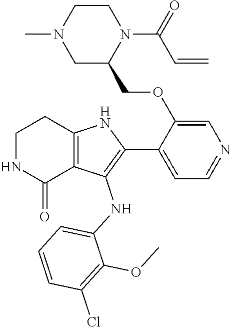 [SMILES: C=CC(=O)N1CCN(C)C[C@@H]1COc1cnccc1-c1[nH]c2c(c1Nc1cccc(Cl)c1OC)C(=O)NCC2] |
| 566 | 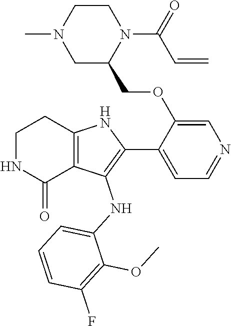 [SMILES: C=CC(=O)N1CCN(C)C[C@@H]1COc1cnccc1-c1[nH]c2c(c1Nc1cccc(F)c1OC)C(=O)NCC2] |
| 567 | 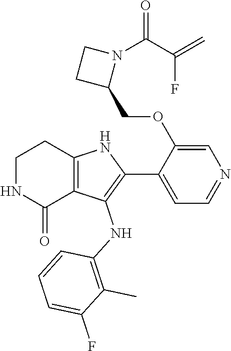 [SMILES: C=C(F)C(=O)N1CC[C@@H]1COc1cnccc1-c1[nH]c2c(c1Nc1cccc(F)c1C)C(=O)NCC2] |
| 568 | 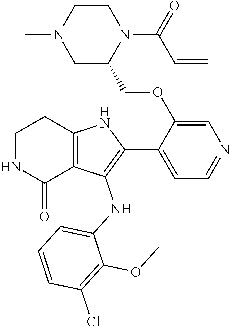 [SMILES: C=CC(=O)N1CCN(C)C[C@H]1COc1cnccc1-c1[nH]c2c(c1Nc1cccc(Cl)c1OC)C(=O)NCC2] |
| 569 | 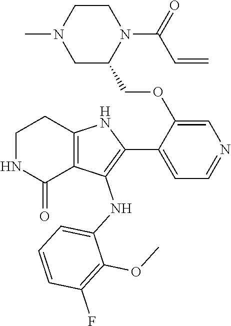 [SMILES: C=CC(=O)N1CCN(C)C[C@H]1COc1cnccc1-c1[nH]c2c(c1Nc1cccc(F)c1OC)C(=O)NCC2] |
| 570 | 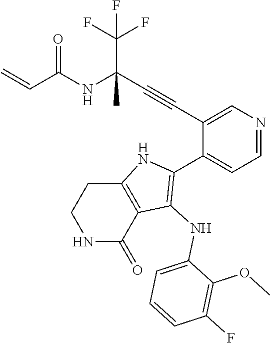 [SMILES: C=CC(=O)N[C@@](C)(C#Cc1cnccc1-c1[nH]c2c(c1Nc1cccc(F)c1OC)C(=O)NCC2)C(F)(F)F] |
| 571 | 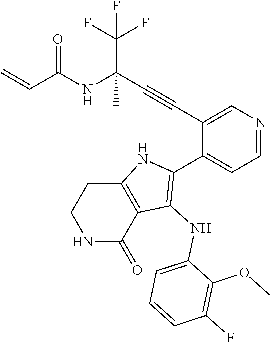 [SMILES: C=CC(=O)N[C@](C)(C#Cc1cnccc1-c1[nH]c2c(c1Nc1cccc(F)c1OC)C(=O)NCC2)C(F)(F)F] |
| 572 | 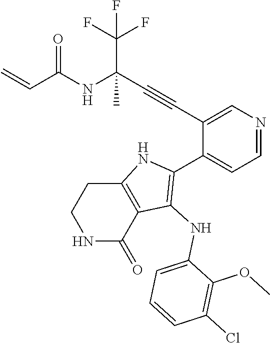 [SMILES: C=CC(=O)N[C@](C)(C#Cc1cnccc1-c1[nH]c2c(c1Nc1cccc(Cl)c1OC)C(=O)NCC2)C(F)(F)F] |
| 573 | 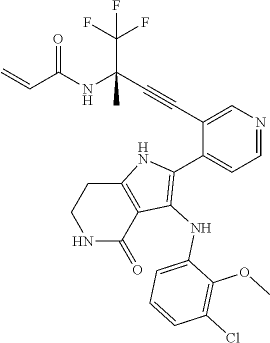 [SMILES: C=CC(=O)N[C@@](C)(C#Cc1cnccc1-c1[nH]c2c(c1Nc1cccc(Cl)c1OC)C(=O)NCC2)C(F)(F)F] |
| 574 | 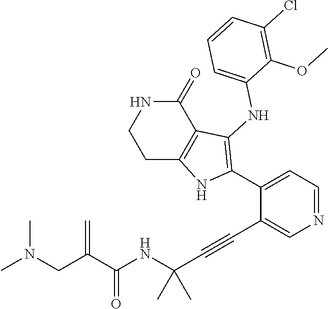 |
| 575 | 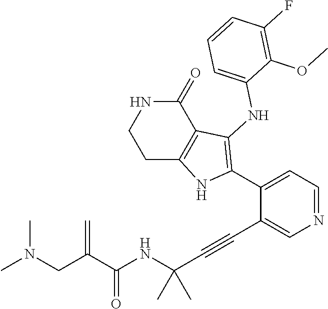 |
| 576 | 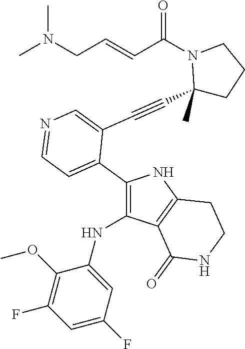 [SMILES: COc1c(F)cc(F)cc1Nc1c(-c2ccncc2C#C[C@@]2(C)CCCN2C(=O)/C=C/CN(C)C)[nH]c2c1C(=O)NCC2] |
| 577 | 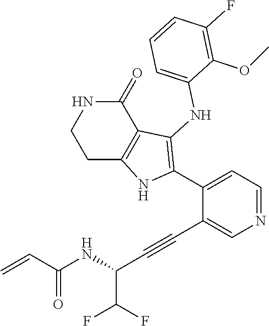 [SMILES: C=CC(=O)N[C@H](C#Cc1cnccc1-c1[nH]c2c(c1Nc1cccc(F)c1OC)C(=O)NCC2)C(F)F] |
| 578 | 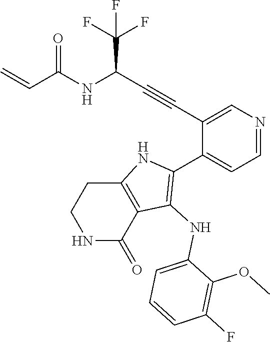 [SMILES: C=CC(=O)N[C@H](C#Cc1cnccc1-c1[nH]c2c(c1Nc1cccc(F)c1OC)C(=O)NCC2)C(F)(F)F] |
| 579 | 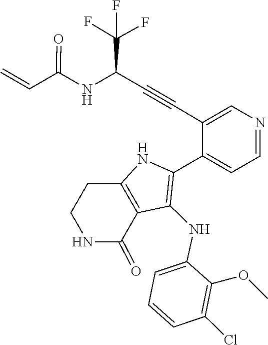 [SMILES: C=CC(=O)N[C@H](C#Cc1cnccc1-c1[nH]c2c(c1Nc1cccc(Cl)c1OC)C(=O)NCC2)C(F)(F)F] |
| 580 | 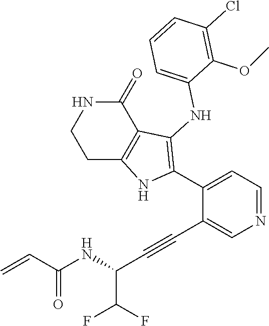 [SMILES: C=CC(=O)N[C@H](C#Cc1cnccc1-c1[nH]c2c(c1Nc1cccc(Cl)c1OC)C(=O)NCC2)C(F)F] |
| 581 | 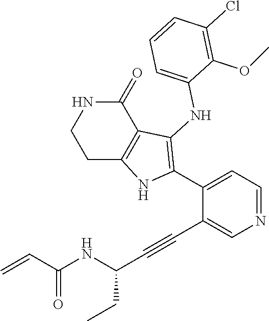 [SMILES: C=CC(=O)N[C@H](C#Cc1cnccc1-c1[nH]c2c(c1Nc1cccc(Cl)c1OC)C(=O)NCC2)CC] |
| 582 | 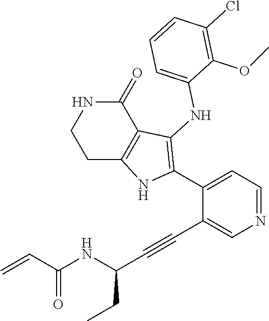 [SMILES: C=CC(=O)N[C@@H](C#Cc1cnccc1-c1[nH]c2c(c1Nc1cccc(Cl)c1OC)C(=O)NCC2)CC] |
| 583 | 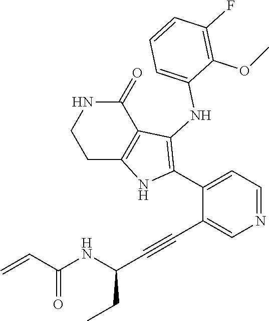 [SMILES: C=CC(=O)N[C@@H](C#Cc1cnccc1-c1[nH]c2c(c1Nc1cccc(F)c1OC)C(=O)NCC2)CC] |
| 584 | 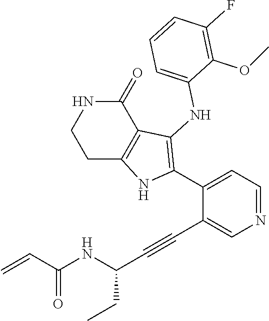 [SMILES: C=CC(=O)N[C@H](C#Cc1cnccc1-c1[nH]c2c(c1Nc1cccc(F)c1OC)C(=O)NCC2)CC] |
| 585 | 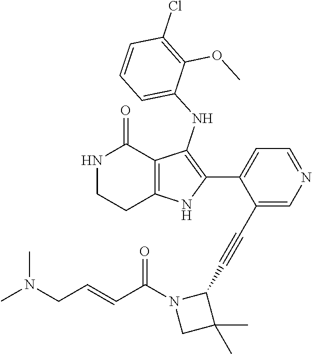 [SMILES: COc1c(Cl)cccc1Nc1c(-c2ccncc2C#C[C@H]2N(C(=O)/C=C/CN(C)C)CC2(C)C)[nH]c2c1C(=O)NCC2] |
| 586 | 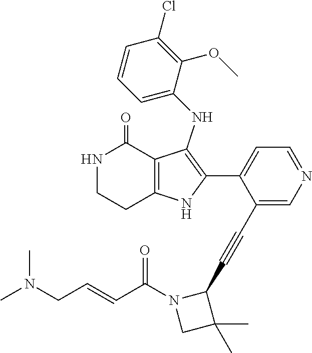 [SMILES: COc1c(Cl)cccc1Nc1c(-c2ccncc2C#C[C@@H]2N(C(=O)/C=C/CN(C)C)CC2(C)C)[nH]c2c1C(=O)NCC2] |
| 587 | 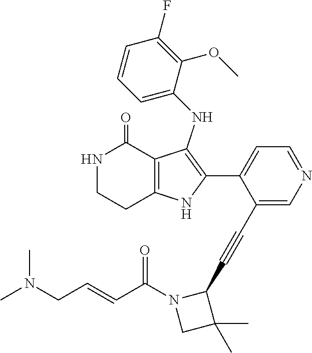 [SMILES: COc1c(F)cccc1Nc1c(-c2ccncc2C#C[C@@H]2N(C(=O)/C=C/CN(C)C)CC2(C)C)[nH]c2c1C(=O)NCC2] |
| 588 | 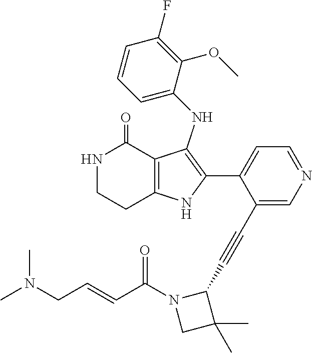 [SMILES: COc1c(F)cccc1Nc1c(-c2ccncc2C#C[C@H]2N(C(=O)/C=C/CN(C)C)CC2(C)C)[nH]c2c1C(=O)NCC2] |
| 589 | 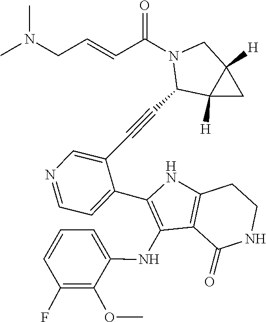 [SMILES: COc1c(F)cccc1Nc1c(-c2ccncc2C#C[C@H]2[C@H]3C[C@H]3CN2C(=O)/C=C/CN(C)C)[nH]c2c1C(=O)NCC2] |
| 590 | 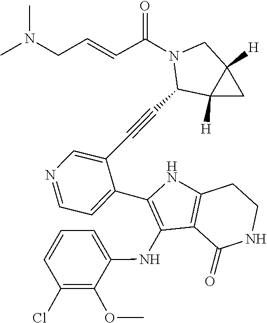 [SMILES: COc1c(Cl)cccc1Nc1c(-c2ccncc2C#C[C@H]2[C@H]3C[C@H]3CN2C(=O)/C=C/CN(C)C)[nH]c2c1C(=O)NCC2] |
| 591 | 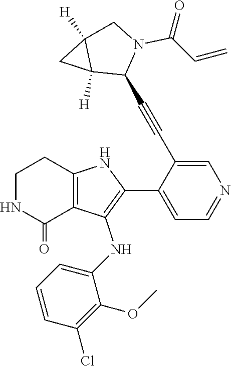 [SMILES: C=CC(=O)N1C[C@@H]2C[C@@H]2[C@@H]1C#Cc1cnccc1-c1[nH]c2c(c1Nc1cccc(Cl)c1OC)C(=O)NCC2] |
| 592 | 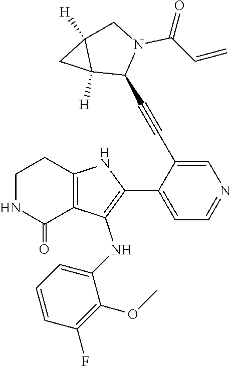 [SMILES: C=CC(=O)N1C[C@@H]2C[C@@H]2[C@@H]1C#Cc1cnccc1-c1[nH]c2c(c1Nc1cccc(F)c1OC)C(=O)NCC2] |
| 593 | 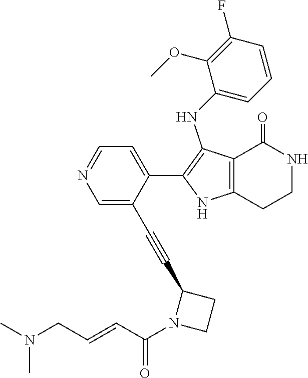 [SMILES: COc1c(F)cccc1Nc1c(-c2ccncc2C#C[C@H]2CCN2C(=O)/C=C/CN(C)C)[nH]c2c1C(=O)NCC2] |
| 594 | 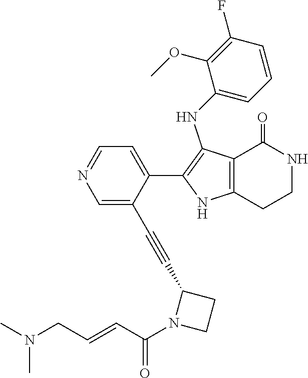 [SMILES: COc1c(F)cccc1Nc1c(-c2ccncc2C#C[C@@H]2CCN2C(=O)/C=C/CN(C)C)[nH]c2c1C(=O)NCC2] |
| 595 | 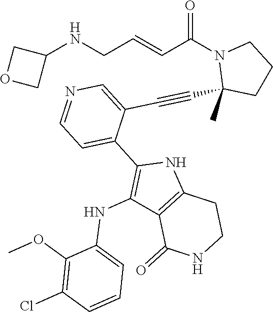 [SMILES: COc1c(Cl)cccc1Nc1c(-c2ccncc2C#C[C@@]2(C)CCCN2C(=O)/C=C/CNC2COC2)[nH]c2c1C(=O)NCC2] |
| 596 | 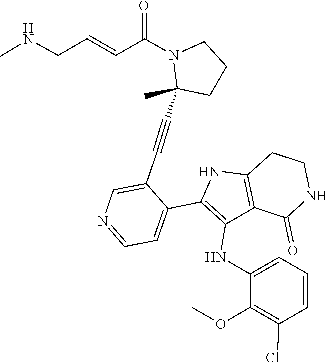 [SMILES: CNC/C=C/C(=O)N1CCC[C@]1(C)C#Cc1cnccc1-c1[nH]c2c(c1Nc1cccc(Cl)c1OC)C(=O)NCC2] |
| 597 | 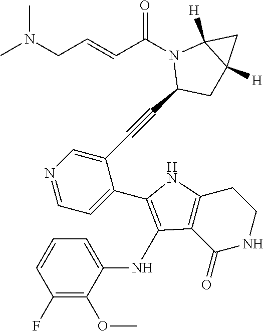 [SMILES: COc1c(F)cccc1Nc1c(-c2ccncc2C#C[C@@H]2C[C@H]3C[C@H]3N2C(=O)/C=C/CN(C)C)[nH]c2c1C(=O)NCC2] |
| 598 | 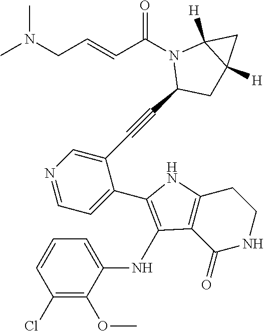 [SMILES: COc1c(Cl)cccc1Nc1c(-c2ccncc2C#C[C@@H]2C[C@H]3C[C@H]3N2C(=O)/C=C/CN(C)C)[nH]c2c1C(=O)NCC2] |
| 599 | 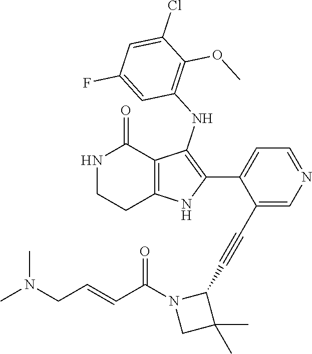 [SMILES: COc1c(Cl)cc(F)cc1Nc1c(-c2ccncc2C#C[C@H]2N(C(=O)/C=C/CN(C)C)CC2(C)C)[nH]c2c1C(=O)NCC2] |
| 600 | 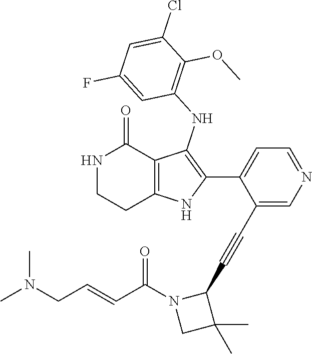 [SMILES: COc1c(Cl)cc(F)cc1Nc1c(-c2ccncc2C#C[C@@H]2N(C(=O)/C=C/CN(C)C)CC2(C)C)[nH]c2c1C(=O)NCC2] |
| 601 | 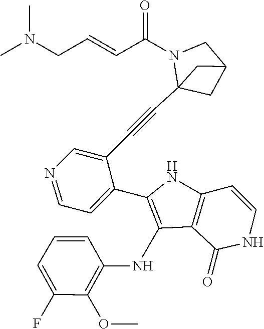 [SMILES: COc1c(F)cccc1Nc1c(-c2ccncc2C#CC23CC(CN2C(=O)/C=C/CN(C)C)C3)[nH]c2cc[nH]c(=O)c12] |
| 602 | 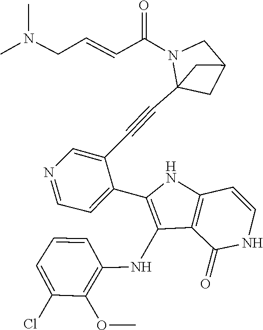 [SMILES: COc1c(Cl)cccc1Nc1c(-c2ccncc2C#CC23CC(CN2C(=O)/C=C/CN(C)C)C3)[nH]c2cc[nH]c(=O)c12] |
| 603 | 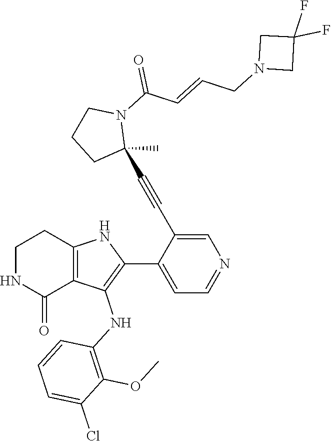 [SMILES: COc1c(Cl)cccc1Nc1c(-c2ccncc2C#C[C@@]2(C)CCCN2C(=O)/C=C/CN2CC(F)(F)C2)[nH]c2c1C(=O)NCC2] |
| 604 | 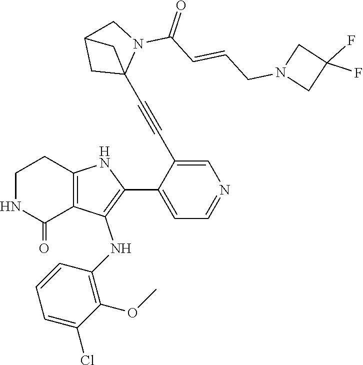 [SMILES: COc1c(Cl)cccc1Nc1c(-c2ccncc2C#CC23CC(CN2C(=O)/C=C/CN2CC(F)(F)C2)C3)[nH]c2c1C(=O)NCC2] |
| 605 | 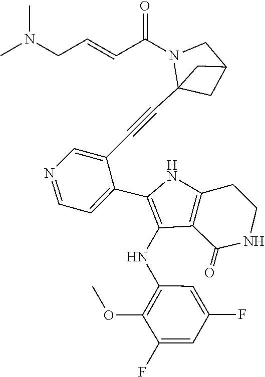 [SMILES: COc1c(F)cc(F)cc1Nc1c(-c2ccncc2C#CC23CC(CN2C(=O)/C=C/CN(C)C)C3)[nH]c2c1C(=O)NCC2] |
| 606 | 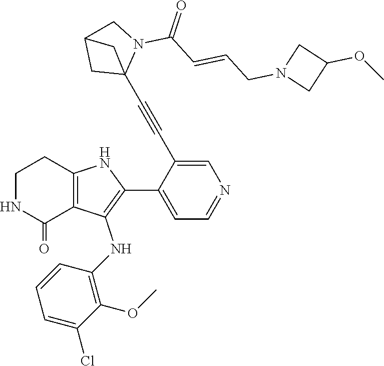 [SMILES: COc1c(Cl)cccc1Nc1c(-c2ccncc2C#CC23CC(CN2C(=O)/C=C/CN2CC(OC)C2)C3)[nH]c2c1C(=O)NCC2] |
| 607 | 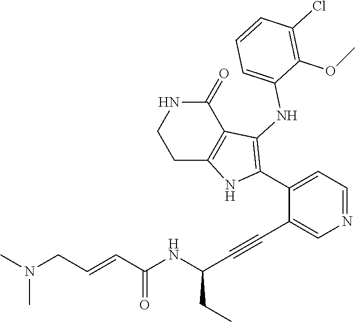 |
| 608 | 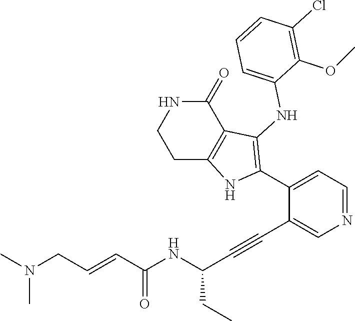 |
| 609 | 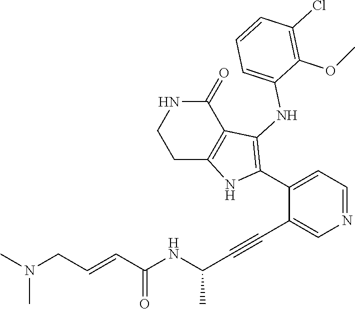 |
| 610 | 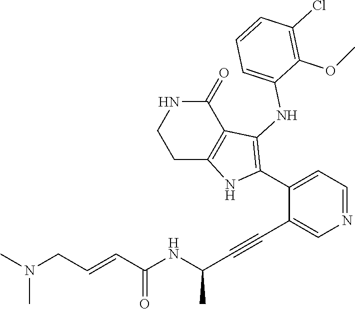 |
| 611 | 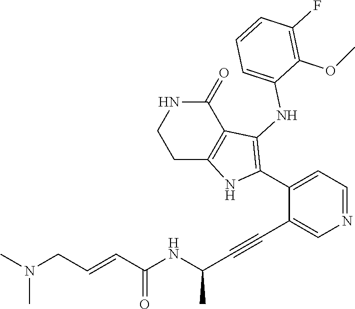 |
| 612 | 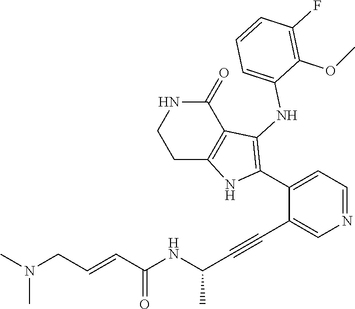 |
| 613 | 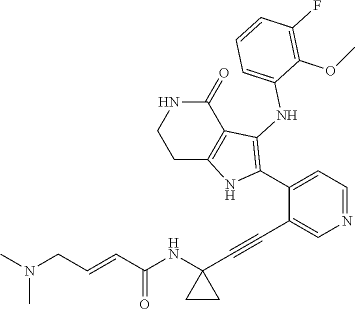 |
| 614 | 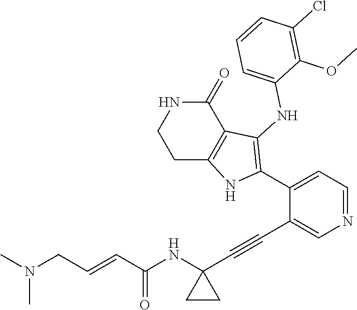 |
| 615 | 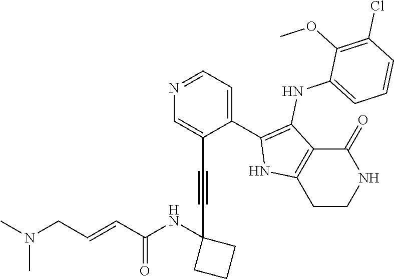 [SMILES: COc1c(Cl)cccc1Nc1c(-c2ccncc2C#CC2(NC(=O)/C=C/CN(C)C)CCC2)[nH]c2c1C(=O)NCC2] |
| 616 | 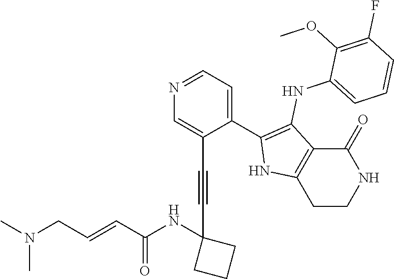 [SMILES: COc1c(F)cccc1Nc1c(-c2ccncc2C#CC2(NC(=O)/C=C/CN(C)C)CCC2)[nH]c2c1C(=O)NCC2] |
| 617 | 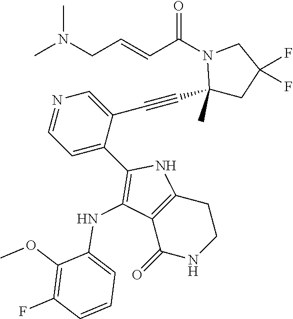 [SMILES: COc1c(F)cccc1Nc1c(-c2ccncc2C#C[C@@]2(C)CC(F)(F)CN2C(=O)/C=C/CN(C)C)[nH]c2c1C(=O)NCC2] |
| 618 | 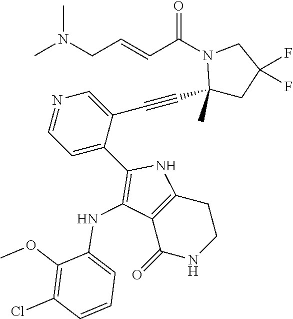 [SMILES: COc1c(Cl)cccc1Nc1c(-c2ccncc2C#C[C@@]2(C)CC(F)(F)CN2C(=O)/C=C/CN(C)C)[nH]c2c1C(=O)NCC2] |
| 619 | 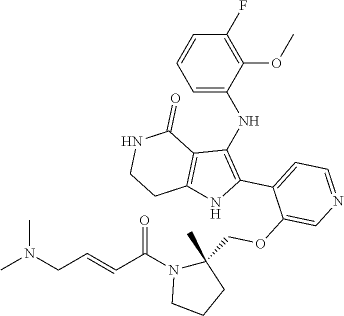 [SMILES: COc1c(F)cccc1Nc1c(-c2ccncc2OC[C@]2(C)CCCN2C(=O)/C=C/CN(C)C)[nH]c2c1C(=O)NCC2] |
| 620 | 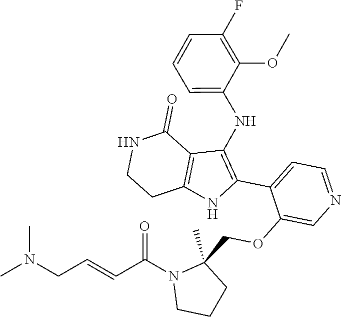 [SMILES: COc1c(F)cccc1Nc1c(-c2ccncc2OC[C@@]2(C)CCCN2C(=O)/C=C/CN(C)C)[nH]c2c1C(=O)NCC2] |
| 621 | 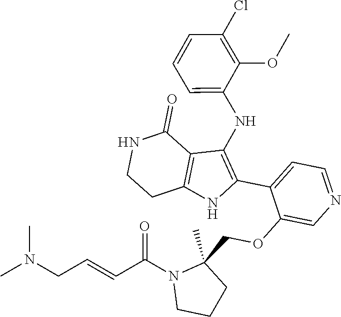 [SMILES: COc1c(Cl)cccc1Nc1c(-c2ccncc2OC[C@@]2(C)CCCN2C(=O)/C=C/CN(C)C)[nH]c2c1C(=O)NCC2] |
| 622 | 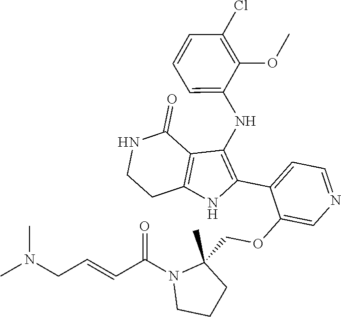 [SMILES: COc1c(Cl)cccc1Nc1c(-c2ccncc2OC[C@]2(C)CCCN2C(=O)/C=C/CN(C)C)[nH]c2c1C(=O)NCC2] |
| 623 | 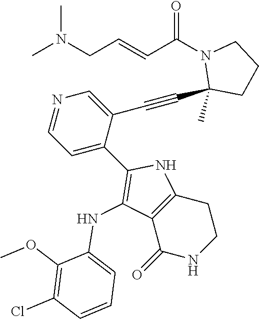 [SMILES: COc1c(Cl)cccc1Nc1c(-c2ccncc2C#C[C@]2(C)CCCN2C(=O)/C=C/CN(C)C)[nH]c2c1C(=O)NCC2] |
| 624 | 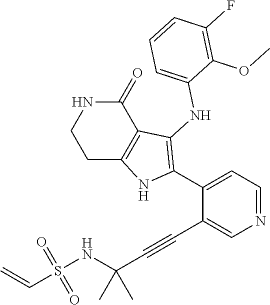 [SMILES: C=CS(=O)(=O)NC(C)(C)C#Cc1cnccc1-c1[nH]c2c(c1Nc1cccc(F)c1OC)C(=O)NCC2] |
| 625 | 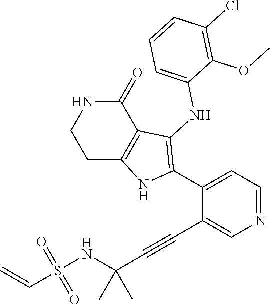 [SMILES: C=CS(=O)(=O)NC(C)(C)C#Cc1cnccc1-c1[nH]c2c(c1Nc1cccc(Cl)c1OC)C(=O)NCC2] |
| 626 | 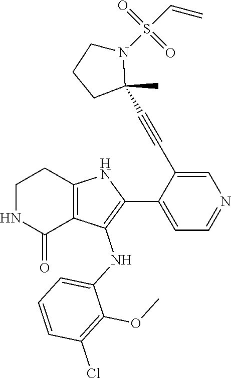 [SMILES: C=CS(=O)(=O)N1CCC[C@@]1(C)C#Cc1cnccc1-c1[nH]c2c(c1Nc1cccc(Cl)c1OC)C(=O)NCC2] |
| 627 | 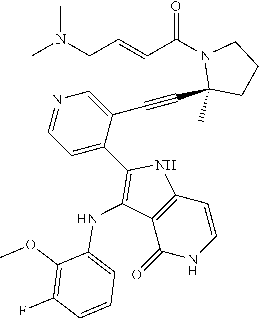 [SMILES: COc1c(F)cccc1Nc1c(-c2ccncc2C#C[C@]2(C)CCCN2C(=O)/C=C/CN(C)C)[nH]c2cc[nH]c(=O)c12] |
| 628 | 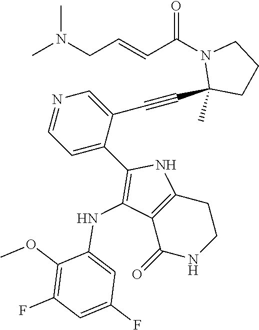 [SMILES: COc1c(F)cc(F)cc1Nc1c(-c2ccncc2C#C[C@]2(C)CCCN2C(=O)/C=C/CN(C)C)[nH]c2c1C(=O)NCC2] |
| 629 | 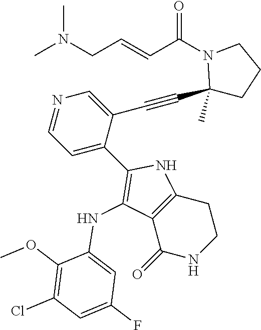 [SMILES: COc1c(Cl)cc(F)cc1Nc1c(-c2ccncc2C#C[C@]2(C)CCCN2C(=O)/C=C/CN(C)C)[nH]c2c1C(=O)NCC2] |
| 630 | 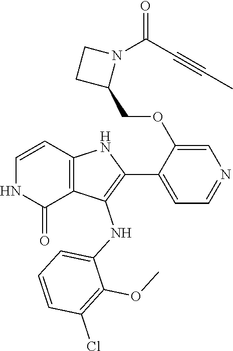 [SMILES: CC#CC(=O)N1CC[C@@H]1COc1cnccc1-c1[nH]c2cc[nH]c(=O)c2c1Nc1cccc(Cl)c1OC] |
| 631 | 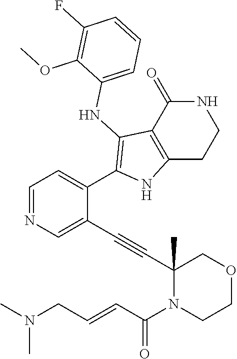 [SMILES: COc1c(F)cccc1Nc1c(-c2ccncc2C#C[C@]2(C)COCCN2C(=O)/C=C/CN(C)C)[nH]c2c1C(=O)NCC2] |
| 632 | 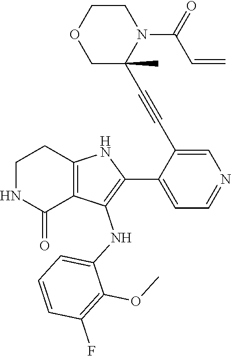 [SMILES: C=CC(=O)N1CCOC[C@@]1(C)C#Cc1cnccc1-c1[nH]c2c(c1Nc1cccc(F)c1OC)C(=O)NCC2] |
| 633 | 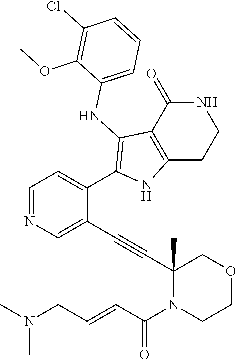 [SMILES: COc1c(Cl)cccc1Nc1c(-c2ccncc2C#C[C@]2(C)COCCN2C(=O)/C=C/CN(C)C)[nH]c2c1C(=O)NCC2] |
| 634 | 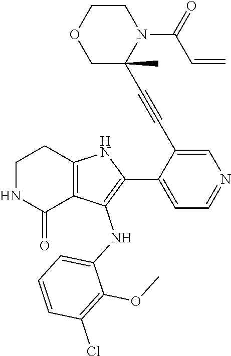 [SMILES: C=CC(=O)N1CCOC[C@@]1(C)C#Cc1cnccc1-c1[nH]c2c(c1Nc1cccc(Cl)c1OC)C(=O)NCC2] |
| 635 | 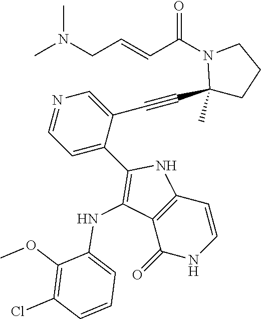 [SMILES: COc1c(Cl)cccc1Nc1c(-c2ccncc2C#C[C@]2(C)CCCN2C(=O)/C=C/CN(C)C)[nH]c2cc[nH]c(=O)c12] |
| 636 | 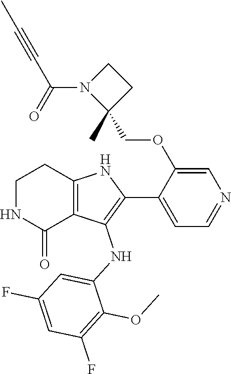 [SMILES: CC#CC(=O)N1CC[C@]1(C)COc1cnccc1-c1[nH]c2c(c1Nc1cc(F)cc(F)c1OC)C(=O)NCC2] |
| 637 | 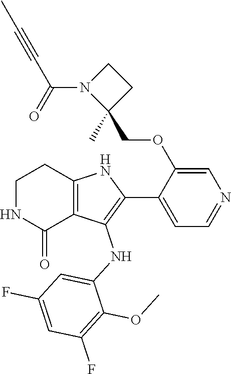 [SMILES: CC#CC(=O)N1CC[C@@]1(C)COc1cnccc1-c1[nH]c2c(c1Nc1cc(F)cc(F)c1OC)C(=O)NCC2] |
| 638 | 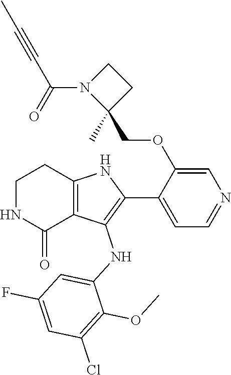 [SMILES: CC#CC(=O)N1CC[C@@]1(C)COc1cnccc1-c1[nH]c2c(c1Nc1cc(F)cc(Cl)c1OC)C(=O)NCC2] |
| 639 | 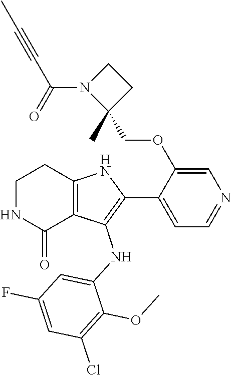 [SMILES: CC#CC(=O)N1CC[C@]1(C)COc1cnccc1-c1[nH]c2c(c1Nc1cc(F)cc(Cl)c1OC)C(=O)NCC2] |
| 640 | 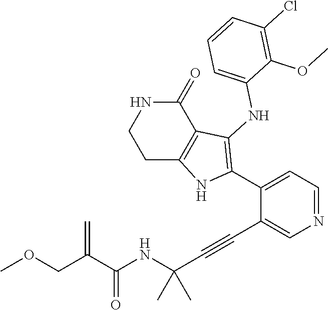 |
| 641 | 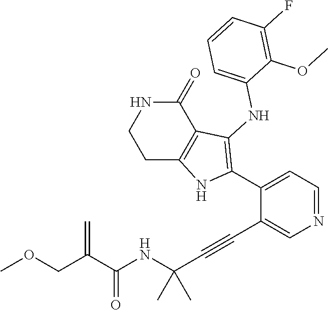 |
| 642 | 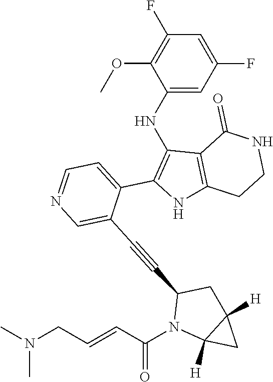 [SMILES: COc1c(F)cc(F)cc1Nc1c(-c2ccncc2C#C[C@H]2C[C@@H]3C[C@@H]3N2C(=O)/C=C/CN(C)C)[nH]c2c1C(=O)NCC2] |
| 643 | 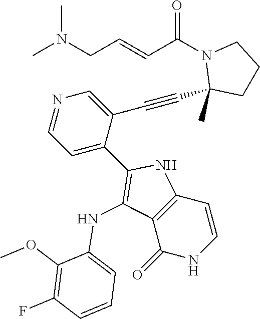 [SMILES: COc1c(F)cccc1Nc1c(-c2ccncc2C#C[C@@]2(C)CCCN2C(=O)/C=C/CN(C)C)[nH]c2cc[nH]c(=O)c12] |
| 644 | 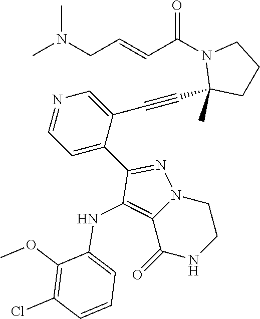 [SMILES: COc1c(Cl)cccc1Nc1c(-c2ccncc2C#C[C@@]2(C)CCCN2C(=O)/C=C/CN(C)C)nn2c1C(=O)NCC2] |
| 645 | 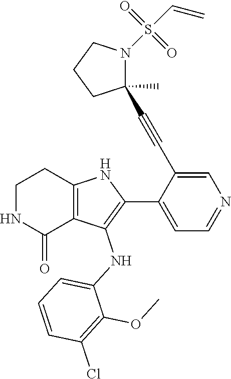 [SMILES: C=CS(=O)(=O)N1CCC[C@]1(C)C#Cc1cnccc1-c1[nH]c2c(c1Nc1cccc(Cl)c1OC)C(=O)NCC2] |
| 646 | 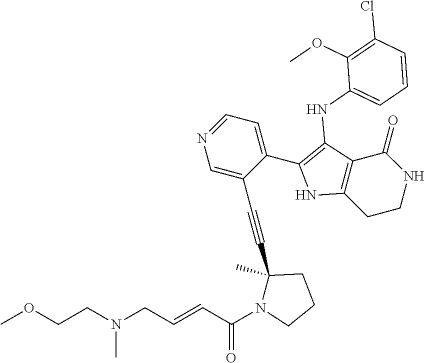 [SMILES: COCCN(C)C/C=C/C(=O)N1CCC[C@]1(C)C#Cc1cnccc1-c1[nH]c2c(c1Nc1cccc(Cl)c1OC)C(=O)NCC2] |
| 647 | 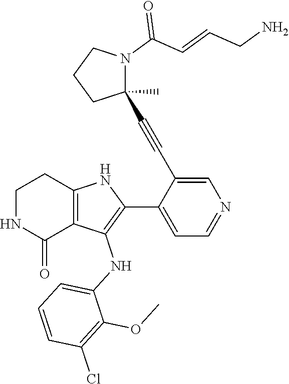 [SMILES: COc1c(Cl)cccc1Nc1c(-c2ccncc2C#C[C@@]2(C)CCCN2C(=O)/C=C/CN)[nH]c2c1C(=O)NCC2] |
| 648 | 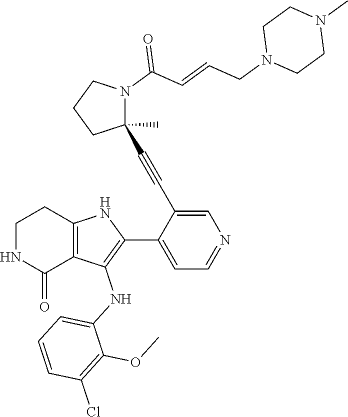 [SMILES: COc1c(Cl)cccc1Nc1c(-c2ccncc2C#C[C@@]2(C)CCCN2C(=O)/C=C/CN2CCN(C)CC2)[nH]c2c1C(=O)NCC2] |
| 649 | 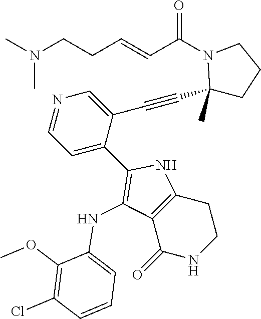 [SMILES: COc1c(Cl)cccc1Nc1c(-c2ccncc2C#C[C@@]2(C)CCCN2C(=O)/C=C/CCN(C)C)[nH]c2c1C(=O)NCC2] |
| 650 | 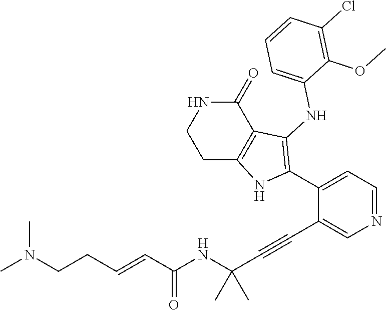 |
| 651 | 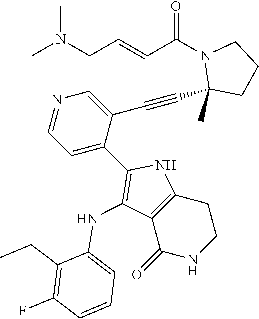 [SMILES: CCc1c(F)cccc1Nc1c(-c2ccncc2C#C[C@@]2(C)CCCN2C(=O)/C=C/CN(C)C)[nH]c2c1C(=O)NCC2] |

Pharmaceutical Compositions and Administration

General

In some embodiments, a chemical entity (e.g., a compound that inhibits EGFR and/or HER2, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

Indications

Provided herein are methods for inhibiting epidermal growth factor receptor tyrosine kinase (EGFR) and/or human epidermal growth factor receptor 2 (HER2). For example, provided herein are inhibitors of EGFR useful for treating or preventing diseases or disorders associated with dysregulation of an EGFR gene, an EGFR kinase, or the expression or activity or level of any of the same (i.e., an EGFR-associated disease or disorder), such as a central nervous system diseases, a pulmonary disorder, cardiovascular disease, ischemia, liver disease, a gastrointestinal disorder, a viral or bacterial infection, an inflammatory and/or autoimmune disease, or cancer (e.g., EGFR-associated cancer). In some embodiments, provided herein are inhibitors of HER2 useful for treating or preventing diseases or disorders associated with dysregulation of a HER2 gene, a HER2 kinase, or expression or activity or level of any of the same, such as cancer (e.g., HER2-associated cancer). In some embodiments, provided herein are inhibitors of EGFR and HER2.

An “EGFR inhibitor” as used herein includes any compound exhibiting EGFR inactivation activity (e.g., inhibiting or decreasing). In some embodiments, an EGFR inhibitor can be selective for an EGFR kinase having one or more mutations. For example, an EGFR inhibitor can bind to the adenosine triphosphate (ATP)-binding site in the tyrosine kinase domain. In some embodiments, an EGFR inhibitor is an allosteric inhibitor.

The compounds provided herein can inhibit EGFR. In some embodiments, the compounds can bind to the EGFR adenosine triphosphate (ATP)-binding site in the tyrosine kinase domain.

The ability of test compounds to act as inhibitors of EGFR may be demonstrated by assays known in the art. The activity of the compounds and compositions provided herein as EGFR inhibitors can be assayed in vitro, in vivo, or in a cell line. In vitro assays include assays that determine inhibition of the kinase and/or ATPase activity. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radioligands. In some cases, an EGFR inhibitor can be evaluated by its effect on the initial velocity of EGFR tyrosine kinase catalyzed peptide phosphorylation (e.g., Yun et al. Cancer Cell. 2007; 11(3):217-227). In some embodiments, the binding constant of an EGFR inhibitor can be determined using fluorescence kinetics (e.g., Yun et al. Cancer Cell. 2007; 11(3):217-227). Examples of surface plasmon resonance (SPR) binding assays include those disclosed in Li, Shiqing, et al. Cancer cell 7.4 (2005): 301-311. Additional EGFR inhibitor assays can be found, for example, in WO 2019/246541 and WO 2019/165358 both of which are incorporated by reference in their entireties).

Assays can include, for example, proliferation inhibition assays such as those that measure cell growth inhibition, such as an MTS assay or by Cell Titer Glo Luminescent Cell viability assay (Promega®). To perform such an assay, cells are seeded and grown in cell culture plates before being exposed to a test compound for varying durations. Assessment of the viability of the cells following this exposure is then performed. Data are normalized with respect to untreated cells and can be displayed graphically. Growth curves can be fitted using a nonlinear regression model with sigmoidal dose response. As another example, a Western Blot analysis can be used. In such assays cells are seeded and grown in culture plates and then treated with a test compound the following day for varying durations. Cells are washed with PBS and lysed. SDS-PAGE gels are used to separate the lysates which are transferred to nitrocellulose membranes, and probed with appropriate antibodies (e.g., phospho-EGFR (Tyrl 068)(3777), total EGFR (2232), p-Akt (Ser473) (4060), total Akt (9272), p-ERK(Thr202/Tyr204)(4370), total ERK (9102), and HSP90 (SC-7947)).

Additional assays can include, for example, assays based on ALPHALISA TECHNOLOGY® (e.g., see the ALPHALISA® EGF/EGFR binding kit from Promega). Such assays use a luminescent oxygen-channeling chemistry to detect molecules of interest in, for example, buffer, cell culture media, serum, and plasma. For example, a biotinylated EGF is bound to streptavidin-coated Alpha donor beads, and EGFR-Fc is captured by anti-human IgG Fc-specific AlphaLISA acceptor beads. When EGF is bound to EGFR, donor beads and acceptor beads come into close proximity, and the excitation of the donor beads provokes the release of singlet oxygen molecules that triggers a cascade of energy transfers in the acceptor beads. This results in a sharp peak of light emission at 615 nm. Such assays can be used, for example, in competitive binding experiments.

Further examples of assays can include assays based on Sox technology (e.g., see the PHOSPHOSENS® Sox-based Homogeneous, Kinetic or Endpoint/Red Fluorescence-based Assays from ASSAYQUANT®). Such assays utilize chelation-enhanced fluorescence (CHEF) using a sulfonamido-oxine (Sox) chromophore in peptide or protein substrates to create real-time sensors of phosphorylation. See, e.g., U.S. Pat. Nos. 8,586,570 and 6,906,194.

Potency of an EGFR inhibitor as provided herein can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining an EGFR-dependent phosphorylation level, in vitro or in vivo (e.g., in tumor cells, A431 cells, Ba/F3 cells, or 3T3 cells expressing a wild type EGFR, a mutant EGFR, or a fragment of any thereof).

Potency of an EGFR inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining an EGFR-dependent phosphorylation level, in vitro or in vivo (e.g., in tumor cells, A431 cells, Ba/F3 cells, or 3T3 cells expressing a wild type EGFR, a mutant EGFR, or a fragment of any thereof).

The selectivity between wild type EGFR and EGFR containing one or more mutations as described herein can also be measured using cellular proliferation assays where cell proliferation is dependent on kinase activity. For example, murine Ba/F3 cells transfected with a suitable version of wild type EGFR (such as VIII; containing a wild type EGFR kinase domain), or Ba/F3 cells transfected with L858R/T790M, Del/T790M/L718Q, L858R/T790M/L718Q, L858R/T790M/C₇₉₇S, Del/T790M/C797S, L858R/T790M/I941R, exon 19 deletion/T790M, or an exon 20 insertion such as V769_D770insX, D770_N771insX, N771_P772insX, P772_H773insX, or H773_V774insX (e.g., A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, or P772_H773insPNP) can be used. Proliferation assays are performed at a range of inhibitor concentrations (e.g., 10 μM, 3 μM, 1.1 μM, 330 nM, 110 nM, 33 nM, 11 nM, 3 nM, 1 nM) and an EC₅₀ is calculated.

An alternative method to measure effects on EGFR activity is to assay EGFR phosphorylation. Wildtype or mutant (L858R/T790M, Del/T790M, Del/T790M/L718Q, L858R/T790M/C797S, Del/T790M/C797S, L858R/T790M/I941R, or L858R/T790M/L718Q) EGFR can be transfected into cells which do not normally express endogenous EGFR and the ability of the inhibitor (e.g., using concentrations as above) to inhibit EGFR phosphorylation can be assayed. Cells are exposed to increasing concentrations of inhibitor and stimulated with EGF. The effects on EGFR phosphorylation are assayed by Western Blotting using phospho-specific EGFR antibodies.

In some embodiments, the compounds provided herein can exhibit potent and selective inhibition of EGFR. For example, the compounds provided herein can bind to the EGFR adenosine triphosphate (ATP)-binding site in the tyrosine kinase domain. In some embodiments, the compounds provided herein can exhibit nanomolar potency against an EGFR kinase including an activating mutation or an EGFR inhibitor resistance mutation, including, for example, the resistance mutations in Table 2a and Table 2b (e.g., L747S, D761Y, T790M, and T854A), with minimal activity against related kinases (e.g., wild type EGFR). Inhibition of wild type EGFR can cause undesireable side effects (e.g., diarrhea and skin rashes) that can impact quality of life and compliance. In some cases, the inhibition of wild type EGFR can lead to dose limiting toxicities. See, e.g., Morphy. J. Med. Chem. 2010, 53, 4, 1413-1437 and Peters. J. Med. Chem. 2013, 56, 22, 8955-8971.

In some embodiments, the compounds of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target an EGFR kinase. For example, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target an EGFR kinase over another kinase or non-kinase target.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit greater inhibition of EGFR containing one or more mutations as described herein (e.g., one or more mutations as described in Table 1a and Table 1b) relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof can exhibit at least 2-fold, 3-fold, 5-fold, 10-fold, 25-fold, 50-fold or 100-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit up to 1000-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit up to 10000-fold greater inhibition of EGFR having a combination of mutations described herein relative to inhibition of wild type EGFR.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 2-fold to about 10-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 10-fold to about 100-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 100-fold to about 1000-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 1000-fold to about 10000-fold greater inhibition of EGFR containing one or more mutations as described herein relative to inhibition of wild type EGFR.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit greater inhibition of EGFR containing one or more mutations as described herein (e.g., one or more mutations as described in Table 1a and Table 1b) relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit at least 2-fold, 3-fold, 5-fold, 10-fold, 25-fold, 50-fold or 100-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit up to 1000-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit up to 10000-fold greater inhibition of EGFR having a combination of mutations described herein relative to inhibition of wild type EGFR.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit from about 2-fold to about 10-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit from about 10-fold to about 100-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit from about 100-fold to about 1000-fold greater inhibition of EGFR containing one or more mutations as described herein relative to inhibition of wild type EGFR. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit from about 1000-fold to about 10000-fold greater inhibition of EGFR containing one or more mutations as described herein relative to inhibition of wild type EGFR.

Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for treating diseases and disorders which can be treated with an EGFR inhibitor, such as EGFR-associated diseases and disorders, e.g., central nervous system diseases (e.g., neurodegenerative diseases), pulmonary disorders, cardiovascular disease, ischemia, liver disease, gastrointestinal disorders, viral or bacterial infections, inflammatory and/or autoimmune diseases (e.g., psoriasis and atopic dermatitis), and proliferative disorders such as cancers, including hematological cancers and solid tumors (e.g., advanced solid tumors).

A “HER2 inhibitor” as used herein includes any compound exhibiting HER2 inactivation activity (e.g., inhibiting or decreasing). In some embodiments, a HER2 inhibitor can be selective for a HER2 kinase having one or more mutations. In some embodiments, a HER2 inhibitor can bind to the HER2 adenosine triphosphate (ATP)-binding site in the tyrosine kinase domain.

The compounds provided herein can inhibit HER2. For example, the compounds can bind to the HER2 adenosine triphosphate (ATP)-binding site in the tyrosine kinase domain. In some embodiments, the compounds provided herein can inhibit wild type HER2. In some embodiments, the compounds provided herein can inhibit HER2 having one or more mutations as described herein.

The ability of test compounds to act as inhibitors of HER2 may be demonstrated by assays known in the art. The activity of the compounds or compositions provided herein as HER2 inhibitors can be assayed in vitro, in vivo, or in a cell line. In vitro assays include assays that determine inhibition of the kinase and/or ATPase activity. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radioligands. In some cases, a HER2 inhibitor can be evaluated by its effect on the initial velocity of HER2 tyrosine kinase catalyzed peptide phosphorylation (e.g., Yun et al. Cancer Cell. 2007; 11(3):217-227). For example, an assay that indirectly measures ADP formed from the HER2 kinase reaction can be used (see, e.g., ATP/NADH coupled assay systems and luminescent kinase assays such as ADP-GLO™ Kinase Assay from Promega). See, e.g., Hanker et al. Cancer Discov. 2017 June; 7(6):575-585; Robichaux et al. Nat Med. 2018 May; 24(5): 638-646; and Yun et al. Proc Natl Acad Sci USA. 2008 Feb. 12; 105(6):2070-5. In some embodiments, an assay that detects substrate phosphorylation using a labeled anti-phospho-tyrosine antibody can be used (see, e.g., Rabindran et al. Cancer Res. 2004 Jun. 1; 64(11):3958-65). In some embodiments, the binding constant of a HER2 inhibitor can be determined using fluorescence kinetics (e.g., Yun et al. Cancer Cell. 2007; 11(3):217-227). Examples of SPR binding assays include those disclosed in Li, Shiqing, et al. Cancer cell 7.4 (2005): 301-311. In some embodiments, covalent binding of a HER2 inhibitor to HER2 can be detected using mass spectrometry, see, e.g., Irie et al. Mol Cancer Ther. 2019 April; 18(4):733-742. Additional HER2 inhibitor assays can be found, for example, in U.S. Pat. No. 9,920,060, WO 2019/241715, and U.S. Publication No. 2017/0166598, each of which are incorporated by reference in their entireties.

Potency of a HER2 inhibitor as provided herein can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining an HER2-dependent phosphorylation level, in vitro or in vivo (e.g., in tumor cells or Ba/F3 cells expressing a wild type HER2, a mutant HER2, or a fragment of any thereof).

Potency of an HER2 inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining an HER2-dependent phosphorylation level, in vitro or in vivo (e.g., in tumor cells or Ba/F3 cells expressing a wild type HER2, a mutant HER2, or a fragment of any thereof).

Assays can include, for example, proliferation inhibition assays such as those that measure cell growth inhibition, such as an MTS assay or by Cell Titer Glo Luminescent Cell viability assay (Promega®). To perform such an assay, cells are seeded and grown in cell culture plates before being exposed to a test compound for varying durations. Assessment of the viability of the cells following this exposure is then performed. Data are normalized with respect to untreated cells and can be displayed graphically. Growth curves can be fitted using a nonlinear regression model with sigmoidal dose response. As another example, a Western Blot analysis can be used. In such assays cells are seeded and grown in culture plates and then treated with a test compound the following day for varying durations. Cells are washed with PBS and lysed. SDS-PAGE gels are used to separate the lysates which are transferred to nitrocellulose membranes, and probed with appropriate antibodies (e.g., phospho-HER2(Tyr1248)(2247), phospho-EGFR-Tyr1173 phospho-HER2-Tyr877, phospho-HER2-Tyr1221, total HER2, phospho-AKT-Thr308, phospho-AKT-Ser374, total AKT, phospho-p44/42 MAPK-Thr202/Tyr204, and p44/42 MAPK).

The selectivity between wild type HER2 and HER2 containing one or more mutations as described herein can also be measured using cellular proliferation assays where cell proliferation is dependent on kinase activity. For example, murine Ba/F3 cells transfected with a suitable version of wild type HER2, or Ba/F3 cells transfected with HER2 having one or more mutations such as S310F, S310Y, R678Q, R678W, R678P, I767M, V773M, V777L, V842I, M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, or P780_Y781insGSP can be used. Proliferation assays are performed at a range of inhibitor concentrations (e.g., 10 μM, 3 μM, 1.1 μM, 330 nM, 110 nM, 33 nM, 11 nM, 3 nM, 1 nM) and an EC₅₀ is calculated.

An alternative method to measure effects on HER2 activity is to assay HER2 phosphorylation. Wildtype or mutant (S310F, S310Y, R678Q, R678W, R678P, I767M, V773M, V777L, V842I, M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, or P780_Y781insGSP) HER2 can be transfected into cells which do not normally express endogenous HER2 and the ability of the inhibitor (e.g., using concentrations as above) to inhibit HER2 phosphorylation can be assayed. Cells are exposed to increasing concentrations of inhibitor and stimulated with EGF. The effects on HER2 phosphorylation are assayed by Western Blotting using phospho-specific HER2 antibodies.

In some embodiments, the compounds provided herein can exhibit potent and selective inhibition of HER2. For example, the compounds provided herein can bind to the HER2 adenosine triphosphate (ATP)-binding site in the tyrosine kinase domain. In some embodiments, the compounds provided herein can exhibit nanomolar potency against a HER2 kinase including an activating mutation or a HER2 inhibitor resistance mutation, including, for example, exon 20 insertions and/or the resistance mutations in Table 5 (e.g., L755S, L755P, T798I, and T798M), with minimal activity against related kinases (e.g., wild type EGFR).

In some embodiments, the compounds of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target a HER2 kinase. For example, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target a HER2 kinase over another kinase (e.g., wild type EGFR) or non-kinase target. It can be desirable to selectively target a HER2 kinase over a wild type EGFR kinase due to undesireable side effects (e.g., diarrhea and skin rashes) that can impact quality of life and compliance. See, e.g., Morphy. J. Med. Chem. 2010, 53, 4, 1413-1437 and Peters. J. Med. Chem. 2013, 56, 22, 8955-8971.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit greater inhibition of wild type HER2 or HER2 containing one or more mutations as described herein (e.g., one or more mutations as described in Table 3) relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof can exhibit at least 2-fold, 3-fold, 5-fold, 10-fold, 25-fold, 50-fold or 100-fold greater inhibition of wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit up to 1000-fold greater inhibition of wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit up to 10000-fold greater inhibition of wild type HER2 or HER2 having a combination of mutations described herein relative to inhibition of another kinase (e.g., wild type EGFR) or non-kinase target.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 2-fold to about 10-fold greater inhibition of wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 10-fold to about 100-fold greater inhibition of wild type HER2 or containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 100-fold to about 1000-fold greater inhibition of wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 1000-fold to about 10000-fold greater inhibition of wild type HER2 or HER2 containing one or more mutations as described herein relative to inhibition of another kinase (e.g., wild type EGFR) or non-kinase target.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit greater inhibition of wild type HER2 or HER2 containing one or more mutations as described herein (e.g., one or more mutations as described in Table 3) relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second HER2 inhibitor can exhibit at least 2-fold, 3-fold, 5-fold, 10-fold, 25-fold, 50-fold or 100-fold greater inhibition of wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second HER2 inhibitor can exhibit up to 1000-fold greater inhibition of wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second HER2 inhibitor can exhibit up to 10000-fold greater inhibition of wild type HER2 or HER2 having a combination of mutations described herein relative to inhibition of another kinase (e.g., wild type EGFR) or non-kinase target.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second HER2 inhibitor can exhibit from about 2-fold to about 10-fold greater inhibition of wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second HER2 inhibitor can exhibit from about 10-fold to about 100-fold greater inhibition of wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second HER2 inhibitor can exhibit from about 100-fold to about 1000-fold greater inhibition of wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second HER2 inhibitor can exhibit from about 1000-fold to about 10000-fold greater inhibition of wild type HER2 or HER2 containing one or more mutations as described herein relative to inhibition of another kinase (e.g., wild type EGFR) or non-kinase target.

Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for treating diseases and disorders which can be treated with a HER2 inhibitor, such as HER2-associated diseases and disorders, e.g., proliferative disorders such as cancers (e.g., a HER2-associated cancer), including hematological cancers and solid tumors (e.g., advanced solid tumors).

In some embodiments, the compounds provided herein can also inhibit EGFR and HER2 as described herein.

In some embodiments, the compounds provided herein can exhibit potent and selective inhibition of EGFR and HER2. In some embodiments, the compounds provided herein can exhibit nanomolar potency against an EGFR kinase having one or more mutations, including, for example, one or more of the mutations in Tables 1a, 1b, 2a and 2b, and a HER2 kinase having one or more mutations, including, for example, the mutations in Table 3, with minimal activity against related kinases (e.g., wild type EGFR).

In some embodiments, the compounds of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target an EGFR and a HER2 kinase. For example, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target an EGFR kinase and a HER2 kinase over another kinase or non-kinase target.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit greater inhibition of EGFR containing one or more mutations as described herein and wild type HER2 or HER2 containing one or more mutations as described herein (e.g., one or more mutations as described in Tables 3-5) relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof can exhibit at least 2-fold, 3-fold, 5-fold, 10-fold, 25-fold, 50-fold or 100-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit up to 1000-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit up to 10000-fold greater inhibition of EGFR containing one or more mutations as described herein and wild type HER2 or HER2 having one or more mutations described herein relative to inhibition of another kinase (e.g., wild type EGFR) or non-kinase target.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 2-fold to about 10-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 10-fold to about 100-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 100-fold to about 1000-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 1000-fold to about 10000-fold greater inhibition of EGFR containing one or more mutations as described herein and wild type HER2 or HER2 containing one or more mutations as described herein relative to inhibition of another kinase (e.g., wild type EGFR) or non-kinase target.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or second HER2 inhibitor can exhibit greater inhibition of EGFR containing one or more mutations as described herein and wild type HER2 or HER2 containing one or more mutations as described herein (e.g., one or more mutations as described in Table 3) relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or second HER2 inhibitor can exhibit at least 2-fold, 3-fold, 5-fold, 10-fold, 25-fold, 50-fold or 100-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or second HER2 inhibitor can exhibit up to 1000-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or HER2 inhibitor can exhibit up to 10000-fold greater inhibition of EGFR containing one or more mutations as described herein and wild type HER2 or HER2 having a combination of mutations described herein relative to inhibition of another kinase (e.g., wild type EGFR) or non-kinase target.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or second HER2 inhibitor can exhibit from about 2-fold to about 10-fold greater inhibition of EGFR containing one or more mutations as described herein and HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or second HER2 inhibitor can exhibit from about 10-fold to about 100-fold greater inhibition of EGFR containing one or more mutations as described herein and HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or second HER2 inhibitor can exhibit from about 100-fold to about 1000-fold greater inhibition of EGFR containing one or more mutations as described herein and second HER2 containing one or more mutations as described herein relative to inhibition of another kinase (e.g., wild type EGFR) or non-kinase target. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or second HER2 inhibitor can exhibit from about 1000-fold to about 10000-fold greater inhibition of EGFR containing one or more mutations as described herein and HER2 containing one or more mutations as described herein relative to inhibition of another kinase (e.g., wild type EGFR) or non-kinase target.

Also provided herein are methods for inhibiting a BUB (budding uninhibited by benzimidazole, BUB1-3) kinase. For example, provided herein are inhibitors of BUB1 kinase useful for treating or preventing diseases or disorders associated with enhanced uncontrolled proliferative cellular processes such as, for example, cancer, inflammation, arthritis, viral diseases, cardiovascular diseases, or fungal diseases. See, for example, WO 2013/050438, WO 2013/092512, WO 2013/167698, WO 2014/147203, WO 2014/147204, WO 2014/202590, WO 2014/202588, WO 2014/202584, WO 2014/202583, WO 2015/063003, WO2015/193339, WO 2016/202755, and WO 2017/021348. In some embodiments, the disease or disorder is cancer.

A “BUB1 inhibitor” as used herein includes any compound exhibiting BUB1 inactivation activity (e.g., inhibiting or decreasing). In some embodiments, a BUB1 inhibitor can be selective for BUB1 over other kinases (e.g., wildtype EGFR).

The compounds provided herein can inhibit a Bub kinase. In some embodiments, the compounds provided herein can inhibit BUB1 kinase.

The ability of test compounds to act as inhibitors of BUB1 may be demonstrated by assays known in the art. The activity of the compounds and compositions provided herein as BUB1 inhibitors can be assayed in vitro, in vivo, or in a cell line. In vitro assays include assays that determine inhibition of the kinase. For example, BUB1 inhibition of a compound provided herein can be determined using a time-resolved fluorescence energy transfer (TR-FRET) assay which measures phosphorylation of a synthetic peptide (e.g., Biotin-AHX-VLLPKKSFAEPG (C-terminus in amide form) by the (recombinant) catalytic domain of human BUB1 (amino acids 704-1085), expressed in Hi5 insect cells with an N-terminal His6-tag and purified by affinity- (Ni-NTA) and size exclusion chromatography. See, for example, WO 2017/021348. In addition, BUB1 activity can be determined at a high ATP concentration using a BUB1 TR-FRET high ATP kinase assay using similar methods as those described above. See, e.g. WO 2019/081486.

In some embodiments, the compounds provided herein exhibit central nervous system (CNS) penetrance. For example, such compounds can be capable of crossing the blood brain barrier (BBB) and inhibiting an EGFR and/or HER2 kinase in the brain and/or other CNS structures. In some embodiments, the compounds provided herein are capable of crossing the blood brain barrier in a therapeutically effective amount. For example, treatment of a patient with cancer (e.g., an EGFR-associated cancer or a HER2-associated cancer such as an EGFR- or HER2-associated brain or CNS cancer or an EGFR-associated or a HER2-associated cancer that has metastasized to the brain or CNS) can include administration (e.g., oral administration) of the compound to the patient.

The ability of the compounds described herein, to cross the BBB can be demonstrated by assays known in the art. Such assays include BBB models such as the transwell system, the hollow fiber (dynamic in vitro BBB) model, other microfluidic BBB systems, the BBB spheroid platform, and other cell aggregate-based BBB models. See, e.g., Cho et al. Nat Commun. 2017; 8: 15623; Bagchi et al. Drug Des Devel Ther. 2019; 13: 3591-3605; Gastfriend et al. Curr Opin Biomed Eng. 2018 March; 5: 6-12; and Wang et al. Biotechnol Bioeng. 2017 January; 114(1): 184-194. In some embodiments, the compounds described herein, are fluorescently labeled, and the fluorescent label can be detected using microscopy (e.g., confocal microscopy). In some such embodiments, the ability of the compound to penetrate the surface barrier of the model can be represented by the fluorescence intensity at a given depth below the surface. In some assays, such as a calcein-AM-based assay, the fluorescent label is non-fluorescent until it permeates live cells and is hydrolyzed by intracellular esterases to produce a fluorescent compound that is retained in the cell and can be quantified with a spectrophotometer. Non-limiting examples of fluorescent labels that can be used in the assays described herein include Cy5, rhodamine, infrared TRDye® CW-800 (LICOR #929-71012), far-red IRDye® 650 (LICOR #929-70020), sodium fluorescein (Na—F), lucifer yellow (LY), 5′carboxyfluorescein, and calcein-acetoxymethylester (calcein-AM). In some embodiments, the BBB model (e.g., the tissue or cell aggregate) can be sectioned, and a compound described herein can be detected in one or more sections using mass spectrometry (e.g., MALDI-MSI analyses). In some embodiments, the ability of a compound described herein to cross the BBB through a transcellular transport system, such as receptor-mediated transport (RMT), carrier-mediated transport (CMT), or active efflux transport (AET), can be demonstrated by assays known in the art. See, e.g., Wang et al. Drug Deliv. 2019; 26(1): 551-565. In some embodiments, assays to determine if compounds can be effluxed by the P-glycoprotein (Pgp) include monolayer efflux assays in which movement of compounds through Pgp is quantified by measuring movement of digoxin, a model Pgp substrate (see, e.g., Doan et al. 2002. J Pharmacol Exp Ther. 303(3):1029-1037). Alternative in vivo assays to identify compounds that pass through the blood-brain barriers include phage-based systems (see, e.g., Peng et al. 2019. ChemRxiv. Preprint doi.org/10.26434/chemrxiv.8242871.v1). In some embodiments, binding of the compounds described herein to brain tissue is quantified. For example, a brain tissue binding assay can be performed using equilibrium dialysis, and the fraction of a compound described herein unbound to brain tissue can be detected using LC-MS/MS (Cyprotex: Brain Tissue Binding Assay www.cyprotex.com/admepk/protein_binding/brain-tissue-binding/).

Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for treating diseases and disorders which can be treated with an EGFR inhibitor, a HER2 inhibitor, a dual EGFR and HER2 inhibitor, and/or a BUB1 inhibitor, such as those described herein, e.g., cancer. Accordingly, provided herein is a method for treating a disease or disorder as provided herein in a subject in need thereof, the method comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt or solvate thereof. In some embodiments, the disease or disorder is cancer.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

In some embodiments, the subject has been identified or diagnosed as having a cancer with a dysregulation of an EGFR gene, an EGFR protein, or expression or activity, or level of any of the same (an EGFR-associated cancer) (e.g., as determined using a regulatory agency-approved, e.g., FDA-approved, assay or kit). In some embodiments, the subject has a tumor that is positive for a dysregulation of an EGFR gene, an EGFR protein, or expression or activity, or level of any of the same (e.g., as determined using a regulatory agency-approved assay or kit). For example, the subject has a tumor that is positive for a mutation as described in Table 1a and Table 1b. The subject can be a subject with a tumor(s) that is positive for a dysregulation of an EGFR gene, an EGFR protein, or expression or activity, or level of any of the same (e.g., identified as positive using a regulatory agency-approved, e.g., FDA-approved, assay or kit). The subject can be a subject whose tumors have a dysregulation of an EGFR gene, an EGFR protein, or expression or activity, or a level of the same (e.g., where the tumor is identified as such using a regulatory agency-approved, e.g., FDA-approved, kit or assay). In some embodiments, the subject is suspected of having an EGFR-associated cancer. In some embodiments, the subject has a clinical record indicating that the subject has a tumor that has a dysregulation of an EGFR gene, an EGFR protein, or expression or activity, or level of any of the same (and optionally the clinical record indicates that the subject should be treated with any of the compositions provided herein).

In some embodiments, the subject has been identified or diagnosed as having a cancer with a dysregulation of a HER2 gene, a HER2 protein, or expression or activity, or level of any of the same (a HER2-associated cancer) (e.g., as determined using a regulatory agency-approved, e.g., FDA-approved, assay or kit). In some embodiments, the subject has a tumor that is positive for a dysregulation of a HER2 gene, a HER2 protein, or expression or activity, or level of any of the same (e.g., as determined using a regulatory agency-approved assay or kit). For example, the subject has a tumor that is positive for a mutation as described in Table 3. The subject can be a subject with a tumor(s) that is positive for a dysregulation of a HER2 gene, a HER2 protein, or expression or activity, or level of any of the same (e.g., identified as positive using a regulatory agency-approved, e.g., FDA-approved, assay or kit). The subject can be a subject whose tumors have a dysregulation of a HER2 gene, a HER2 protein, or expression or activity, or a level of the same (e.g., where the tumor is identified as such using a regulatory agency-approved, e.g., FDA-approved, kit or assay). In some embodiments, the subject is suspected of having a HER2-associated cancer. In some embodiments, the subject has a clinical record indicating that the subject has a tumor that has a dysregulation of a HER2 gene, a HER2 protein, or expression or activity, or level of any of the same (and optionally the clinical record indicates that the subject should be treated with any of the compositions provided herein).

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First LR. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In certain embodiments, compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for preventing diseases and disorders as defined herein (for example, autoimmune diseases, inflammatory diseases, pulmonary disorders, cardiovascular disease, ischemia, liver disease, gastrointestinal disorders, viral or bacterial infections, central nervous system diseases (e.g., neurodegenerative diseases), and cancer). The term “preventing” as used herein means to delay the onset, recurrence or spread, in whole or in part, of the disease or condition as described herein, or a symptom thereof.

The term “EGFR-associated disease or disorder” as used herein refers to diseases or disorders associated with or having a dysregulation of an EGFR gene, an EGFR kinase (also called herein an EGFR kinase protein), or the expression or activity or level of any (e.g., one or more) of the same (e.g., any of the types of dysregulation of an EGFR gene, an EGFR kinase, an EGFR kinase domain, or the expression or activity or level of any of the same described herein). Non-limiting examples of an EGFR-associated disease or disorder include, for example, cancer, a central nervous system disease, a pulmonary disorder, cardiovascular disease, ischemia, liver disease, a gastrointestinal disorder, a viral or bacterial infection, and an inflammatory and/or autoimmune disease (e.g., psoriasis, eczema, atopic dermatitis, and atherosclerosis).

In some embodiments of any of the methods or uses described herein, the inflammatory and/or autoimmune disease is selected from arthritis, systemic lupus erythematosus, atherosclerosis, and skin related disorders such as psoriasis, eczema, and atopic dermatitis. See, e.g., Wang et al. Am J Transl Res. 2019; 11(2): 520-528; Starosyla et al. World J Pharmacol. Dec. 9, 2014; 3(4): 162-173; Choi et al. Biomed Res Int. 2018 May 15; 2018:9439182; and Wang et al. Sci Rep. 2017; 7: 45917.

In some embodiments of any of the methods or uses described herein, the central nervous system disease is a neurodegenerative disease. In some embodiments, the central nervous system disease is selected from Alzheimer's disease, Parkinson's disease, Huntington's disease, amyotrophic lateral sclerosis, spinal cord injury, peripheral neuropathy, brain ischemia, and a psychiatric disorder such as schizophrenia. See, e.g., Iwakura and Nawa. Front Cell Neurosci. 2013 Feb. 13; 7:4; and Chen et al. Sci Rep. 2019 Feb. 21; 9(1):2516.

The term “EGFR-associated cancer” as used herein refers to cancers associated with or having a dysregulation of an EGFR gene, an EGFR kinase (also called herein an EGFR kinase protein), or expression or activity, or level of any of the same. Non-limiting examples of an EGFR-associated cancer are described herein.

The phrase “dysregulation of an EGFR gene, an EGFR kinase, or the expression or activity or level of any of the same” refers to a genetic mutation (e.g., a mutation in an EGFR gene that results in the expression of an EGFR protein that includes a deletion of at least one amino acid as compared to a wild type EGFR protein, a mutation in an EGFR gene that results in the expression of an EGFR protein with one or more point mutations as compared to a wild type EGFR protein, a mutation in an EGFR gene that results in the expression of an EGFR protein with at least one inserted amino acid as compared to a wild type EGFR protein, a gene duplication that results in an increased level of EGFR protein in a cell, or a mutation in a regulatory sequence (e.g., a promoter and/or enhancer) that results in an increased level of EGFR protein in a cell), an alternative spliced version of an EGFR mRNA that results in an EGFR protein having a deletion of at least one amino acid in the EGFR protein as compared to the wild type EGFR protein), or increased expression (e.g., increased levels) of a wild type EGFR kinase in a mammalian cell due to aberrant cell signaling and/or dysregulated autocrine/paracrine signaling (e.g., as compared to a control non-cancerous cell). As another example, a dysregulation of an EGFR gene, an EGFR protein, or expression or activity, or level of any of the same, can be a mutation in an EGFR gene that encodes an EGFR protein that is constitutively active or has increased activity as compared to a protein encoded by an EGFR gene that does not include the mutation. Non-limiting examples of EGFR kinase protein point mutations/insertions/deletions are described in Table 1a and Table 1b. Additional examples of EGFR kinase protein mutations (e.g., point mutations) are EGFR inhibitor resistance mutations (e.g., EGFR inhibitor mutations). Non-limiting examples of EGFR inhibitor resistance mutations are described in Table 2a and Table 2b. For example, the one or more EGFR inhibitor resistance mutations can include a substitution at amino acid position 718, 747, 761, 790, 797, or 854 (e.g., L718Q, L747S, D761Y, T790M, C797S, or T854A). Such mutation and overexpression is associated with the development of a variety of cancers (Shan et al., Cell 2012, 149(4) 860-870).

In some embodiments, dysregulation of an EGFR gene, an EGFR kinase, or the expression or activity or level of any of the same can be caused by an activating mutation in an EGFR gene. In some embodiments, dysregulation of an EGFR gene, an EGFR kinase, or the expression or activity or level of any of the same can be caused by a genetic mutation that results in the expression of an EGFR kinase that has increased resistance to an EGFR inhibitor, a tyrosine kinase inhibitor (TKI), and/or a multi-kinase inhibitor (MKI), e.g., as compared to a wild type EGFR kinase (see, e.g., the amino acid substitutions in Table 2a and Table 2b). In some embodiments, dysregulation of an EGFR gene, an EGFR kinase, or the expression or activity or level of any of the same can be caused by a mutation in a nucleic acid encoding an altered EGFR protein (e.g., an EGFR protein having a mutation (e.g., a primary mutation)) that results in the expression of an altered EGFR protein that has increased resistance to inhibition by an EGFR inhibitor, a tyrosine kinase inhibitor (TKI), and/or a multi-kinase inhibitor (MKI), e.g., as compared to a wild type EGFR kinase (see, e.g., the amino acid substitutions in Table 2a and Table 2b). The exemplary EGFR kinase point mutations, insertions, and deletions shown in Tables 1a, 1b, 2a and 2b can be caused by an activating mutation and/or can result in the expression of an EGFR kinase that has increased resistance to an EGFR inhibitor), tyrosine kinase inhibitor (TKI), and/or a multi-kinase inhibitor (MKI).

In some embodiments, the individual has two or more EGFR inhibitor resistance mutations that increase resistance of the cancer to a first EGFR inhibitor. For example, the individual can have two EGFR inhibitor resistance mutations. In some embodiments, the two mutations occur in the same EGFR protein. In some embodiments, the two mutations occur in separate EGFR proteins. In some embodiments, the individual can have three EGFR inhibitor resistance mutations. In some embodiments, the three mutations occur in the same EGFR protein. In some embodiments, the three mutations occur in separate EGFR proteins. For example, the individual has two or more EGFR inhibitor resistance mutations selected from Del 19/L718Q, Del 19/T790M, Del 19/L844V, Del 19/T790M/L718Q, Del/T790M/C797S, Del 19/T790M/L844V, L858R/L718Q, L858R/L844V, L858R/T790M, L858R/T790M/L718Q, L858R/T790M/C797S, and L858R/T790M/I941R, or any combination thereof, e.g., any two of the aforementioned EGFR inhibitor resistance mutations.

The term “activating mutation” in reference to EGFR describes a mutation in an EGFR gene that results in the expression of an EGFR kinase that has an increased kinase activity, e.g., as compared to a wild type EGFR kinase, e.g., when assayed under identical conditions. For example, an activating mutation can be a mutation in an EGFR gene that results in the expression of an EGFR kinase that has one or more (e.g., two, three, four, five, six, seven, eight, nine, or ten) amino acid substitutions (e.g., any combination of any of the amino acid substitutions described herein) that has increased kinase activity, e.g., as compared to a wild type EGFR kinase, e.g., when assayed under identical conditions. In another example, an activating mutation can be a mutation in an EGFR gene that results in the expression of an EGFR kinase that has one or more (e.g., two, three, four, five, six, seven, eight, nine, or ten) amino acids deleted, e.g., as compared to a wild type EGFR kinase, e.g., when assayed under identical conditions. In another example, an activating mutation can be a mutation in an EGFR gene that results in the expression of an EGFR kinase that has at least one (e.g., at least 2, at least 3, at least 4, at least 5, at least 6, at least 7, at least 8, at least 9, at least 10, at least 12, at least 14, at least 16, at least 18, or at least 20) amino acid inserted as compared to a wild type EGFR kinase, e.g., the exemplary wild type EGFR kinase described herein, e.g., when assayed under identical conditions. Additional examples of activating mutations are known in the art.

The term “wild type” or “wild-type” describes a nucleic acid (e.g., an EGFR gene or an EGFR mRNA) or protein (e.g., an EGFR protein) sequence that is typically found in a subject that does not have a disease or disorder related to the reference nucleic acid or protein.

The term “wild type EGFR” or “wild-type EGFR” describes an EGFR nucleic acid (e.g., an EGFR gene or an EGFR mRNA) or protein (e.g., an EGFR protein) that is found in a subject that does not have an EGFR-associated disease, e.g., an EGFR-associated cancer (and optionally also does not have an increased risk of developing an EGFR-associated disease and/or is not suspected of having an EGFR-associated disease), or is found in a cell or tissue from a subject that does not have an EGFR-associated disease, e.g., an EGFR-associated cancer (and optionally also does not have an increased risk of developing an EGFR-associated disease and/or is not suspected of having an EGFR-associated disease).

Provided herein is a method of treating cancer (e.g., an EGFR-associated cancer) in a subject in need of such treatment, the method comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. For example, provided herein are methods for treating an EGFR-associated cancer in a subject in need of such treatment, the method comprising a) detecting a dysregulation of an EGFR gene, an EGFR kinase, or the expression or activity or level of any of the same in a sample from the subject; and b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the dysregulation of an EGFR gene, an EGFR kinase, or the expression or activity or level of any of the same includes one or more EGFR kinase protein point mutations/insertions. Non-limiting examples of EGFR kinase protein point mutations/insertions/deletions are described in Table 1a and 1b. In some embodiments, the EGFR kinase protein point mutations/insertions/deletions are selected from the group consisting of G719S, G719C, G719A, L747S, D761Y, T790M, T854A, L858R, L861Q, a deletion in exon 19 (e.g., L747_A750del), and an insertion in exon 20 (e.g., V769_D770insX, D770_N771insX, N771_P772insX, P772_H773insX, or H773_V774insX). In some embodiments, the EGFR kinase protein point mutations/insertions/deletions are selected from the group consisting of L858R, deletions in exon 19 (e.g., L747_A750del), L747S, D761Y, T790M, and T854A. In some embodiments, the EGFR kinase protein insertion is an exon 20 insertion. In some embodiments, the EGFR kinase protein insertion is an exon 20 insertion selected from the group consisting of: V769_D770insX, D770_N771insX, N771_P772insX, P772_H773insX, and H773_V774insX. For example, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP; or any combination thereof, e.g., any two or more independently selected exon 20 insertions; e.g., any two independently selected exon 20 insertions (e.g., V769_D770insASV and D770_N771insSVD).

In some embodiments of any of the methods or uses described herein, the cancer (e.g., EGFR-associated cancer) is selected from a hematological cancer (e.g., acute lymphocytic cancer, Hodgkin lymphoma, non-Hodgkin lymphoma, and leukemia such as acute-myelogenous leukemia (AML), chronic-myelogenous leukemia (CML), acute-promyelocytic leukemia, and acute lymphocytic leukemia (ALL)), central or peripheral nervous system tissue cancer, an endocrine or neuroendocrine cancer including multiple neuroendocrine type I and type II tumors, Li-Fraumeni tumors, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, oral cancer, oropharyngeal cancer, nasopharyngeal cancer, respiratory cancer, urogenital cancer, cancer of the vulva, colon cancer, esophageal cancer, tracheal cancer, cervical cancer, gastrointestinal carcinoid tumor, hypopharynx cancer, kidney cancer, larynx cancer, liver cancer, lung cancer, malignant mesothelioma, melanoma, multiple myeloma, nasopharynx cancer, ovarian cancer, pancreatic cancer including pancreatic islet cell cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, parathyroid cancer, pituitary tumors, adrenal gland tumors, ureter cancer, biliary cancer, and urinary bladder cancer. In some embodiments, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In some embodiments, the cancer is pancreatic cancer, head and neck cancer, melanoma, colon cancer, renal cancer, leukemia, lung cancer, or breast cancer. In some cases, the cancer is melanoma, colon cancer, renal cancer, leukemia, or breast cancer.

In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, Liu et al. J Exp Clin Cancer Res. 2019 May 23; 38(1):219); and Ding et al. Cancer Res. 2003 Mar. 1; 63(5):1106-13). In some embodiments, the brain tumor is a primary brain tumor. In some embodiments, the brain tumor is a metastatic brain tumor, e.g., a metastatic brain tumor from lung cancer, melanoma, breast cancer, ovarian cancer, colorectal cancer, kidney cancer, bladder cancer, or undifferentiated carcinoma. In some embodiments, the brain tumor is a metastatic brain tumor from lung cancer (e.g., non-small cell lung cancer). In some embodiments, the compounds provided herein exhibit brain and/or central nervous system (CNS) penetrance. In some embodiments, the patient has previously been treated with another anticancer agent, e.g., another EGFR and/or HER2 inhibitor (e.g., a compound that is not a compound of Formula I) or a multi-kinase inhibitor.

In some embodiments, the cancer is a cancer of B cell origin. In some embodiments, the cancer is a lineage dependent cancer. In some embodiments, the cancer is a lineage dependent cancer where EGFR or the dysregulation of an EGFR gene, an EGFR kinase, or expression or activity or level of any of the same, plays a role in the initiation and/or development of the cancer.

In some embodiments, the cancer is an EGFR-associated cancer. Accordingly, also provided herein is a method for treating a subject diagnosed with or identified as having an EGFR-associated cancer, e.g., any of the exemplary EGFR-associated cancers disclosed herein, comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof, as defined herein.

In some embodiments, the dysregulation of an EGFR gene, an EGFR kinase, or expression or activity or level of any of the same, includes one or more deletions (e.g., deletion of an amino acid at position 4), insertions, or point mutation(s) in an EGFR kinase. In some embodiments, dysregulation of an EGFR gene, an EGFR kinase, or expression or activity or level of any of the same, includes at least one deletion, insertion, or point mutation in an EGFR gene that results in the production of an EGFR kinase that has one or more of the amino acid substitutions, insertions, or deletions in Table 1a and Table 1b. In some embodiments, the dysregulation of an EGFR gene, an EGFR kinase, or expression or activity or level of any of the same, includes a deletion of one or more residues from the EGFR kinase, resulting in constitutive activity of the EGFR kinase domain.

In some embodiments, the dysregulation of an EGFR gene, an EGFR kinase, or expression or activity or level of any of the same, includes at least one point mutation in an EGFR gene that results in the production of an EGFR kinase that has one or more amino acid substitutions, insertions, or deletions as compared to the wild type EGFR kinase (see, for example, the point mutations listed in Table 1a and Table 1b). In some embodiments, dysregulation of an EGFR gene, an EGFR kinase, or expression or activity or level of any of the same, includes at least one point mutation in an EGFR gene that results in the production of an EGFR kinase that has one or more of the amino acid substitutions, insertions, or deletions in Table 1a and Table 1b.

In some embodiments, the dysregulation of an EGFR gene, an EGFR kinase, or expression or activity or level of any of the same, includes an insertion of one or more residues in exon 20 of the EGFR gene (e.g., any of the exon 20 insertions described in Table 1a and Table 1b). Exon 20 of EGFR has two major regions, the c -helix (residues 762-766) and the loop following the c-helix (residues 767-774). Studies suggest that for some exon 20 insertions (e.g., insertions after residue 764), a stabilized and ridged active conformation induces resistance to first generation EGFR inhibitors. In some embodiments, the dysregulation of an EGFR gene, an EGFR kinase, or expression or activity or level of any of the same, includes an insertion of one or more residues in exon selected from the group consisting of: V769_D770insX, D770_N771insX, N771_P772insX, P772_H773insX, and H773_V774insX. For example, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP; or any combination thereof, e.g., any two or more independently selected exon 20 insertions; e.g., any two independently selected exon 20 insertions (e.g., V769_D770insASV and D770_N771insSVD).

TABLE 1a
EGFR Protein Amino Acid Substitutions/Insertions/Deletions^{A}

| | | Non-limiting |
|---|---|---|
| | Non-Limiting | Exemplary |
| Amino Acid | Exemplary | EGFR-associated |
| Position(s) | Mutations | Cancer(s) |
| 62 | L62R^{11, B} | |
| 108 | R108K^{11, B} | |
| 216 | A216T^{11, B} | |
| 222 | R222C^{11, B} | |
| 252 | R252C^{11, B} | |
| 289 | A289D, A289T, A289V^{11, B} | |
| 292 | V292L^{11, B} | |
| 304 | H304Y^{11, B} | |
| 306 | S306L^{11, B} | |
| 492 | S492R^{11, B} | |
| 596 | P596L^{11, B} | |
| 598 | G598V^{11, B} | |
| 688 | L688F⁶ | Lung |
| | | adenocarcinoma⁶ |
| 689 | V689L⁶ | Lung |
| | | adenocarcinoma⁶ |
| 703 | L703I, L703P^{11, B} | |
| 706 | I706T⁶ | Lung |
| | | adenocarcinoma⁶ |
| 709 | E709A, E709G, E709K, E709H, | Lung adeno- |
| | E709V^{2, 6, 9} | carcinoma^{2, 6}, |
| | | NSCLC⁹ |
| 709-710 | E709_T710delinsD⁹ | NSCLC⁹ |
| (sometimes | | |
| also called | | |
| exon 18 | | |
| deletion or | | |
| del_18) | | |
| 714 | K714R^{11, B} | |
| 718 | L718Q^{1, 11, B} | |
| 719 | G719S, G719C, G719A, G719D^{1, 4} | NSCLC³ |
| 719 and 706 | G719A/I706T⁶ | Lung |
| | | adenocarcinoma⁶ |
| 719 and 709 | G719S/E709K⁶, G719S/E709A^{11, B}, | Lung |
| | G719C/E709A^{11, B} | adenocarcinoma⁶ |
| 719 and | G719X/del_18⁹ | NSCLC⁹ |
| exon | | |
| 18 deletion | | |
| 720 | S720F^{11, B} | |
| 735 | G735S^{11, B} | |
| 741 | P741L^{11, B} | |
| 744 | I744M^{11, B} | |
| 747 | L747S^{11, B} | |
| 749 | E749Q⁶ | Lung |
| | | adenocarcinoma⁶ |
| 750 | A750P^{5, 10} | NSCLC⁵, lung |
| | | adeno- |
| | | carcinoma¹⁰ |
| 750 and 749 | A750P/E749Q⁶ | |
| 751 | T751I^{11, B} | |
| 752 | S752F¹⁰ | Lung adeno- |
| | | carcinoma¹⁰ |
| 753 | P753S^{11, B} | |
| 765 | V765A³, V765M^{11, B} | NSCLC³ |
| 767 | A767V^{11, B} | |
| 768 | S768I^{2, 9} | Lung |
| | | adenocarcinoma², |
| | | NSCLC⁹ |
| 768 and 719 | S768I/G719A^{11, B}, | |
| | S768I/G719C^{11, B}, | |
| | S768I/G719S^{11, B}, | |
| 769 | V769M3, V769L^{11, B} | |
| 771 | N771F⁶ | Lung |
| | | adenocarcinoma⁶ |
| 773 | H773Y⁶, H773L^{11, B}, H773Y^{11, B} | Lung |
| | | adenocarcinoma⁶ |
| 774 | V774M, V774A^{2, 3} | Lung adeno- |
| | | carcinoma², |
| | | NSCLC³ |
| 774 and 773 | V774M/H773L¹⁰ | NSCLC¹⁰ |
| 776 | R776H, R776C⁶, R776G^{11, B} | Lung |
| | | adenocarcinoma⁶ |
| 776 and 719 | R776H/G719S⁶ | Lung |
| | | adenocarcinoma⁶ |
| 779 | G779S^{11, B} | |
| 783 | T783A³ | NSCLC³ |
| 784 | S784P³ | |
| 785 | T785I⁶ | Lung |
| | | adenocarcinoma⁶ |
| 786 | V786M^{11, B} | |
| 790 | T790M³ | NSCLC³ |
| 790 and 719 | T790M/G719A^{11, B} | |
| 790 and 948 | T790M/V948R¹ | |
| 797 and 790 | C797S/T790M^{11, B} | |
| 798 | P798H^{11, B} | |
| 802 | V802I^{11, B} | |
| 813 | Y813H⁶ | Lung |
| | | adenocarcinoma⁶ |
| 824 | G824S⁶ | Lung |
| | | adenocarcinoma⁶ |
| 824 and 688 | G824S/L688F⁶ | Lung |
| | | adenocarcinoma⁶ |
| 831 | R831H, R831L^{11, B} | |
| 833 | L833V^{2, 8}, L833F^{11, B} | Lung |
| | | adenocarcinoma², |
| | | NSCLC⁸ |
| 834 | V834L, V834M^{11, B} | |
| 835 | H835L^{11, B} | |
| 835, 833, | H835L/L833V/R670W⁸ | NSCLC⁸ |
| and 670 | | |
| 838 | L838V^{11, B} | |
| 843 | V843I³ | NSCLC³ |
| 844 | L844V¹ | |
| 845 | V845M⁶ | Lung |
| | | adenocarcinoma⁶ |
| 848 | P848L^{11, B} | |
| 851 | V851I⁶ | Lung |
| | | adenocarcinoma⁶ |
| 854 | T854A^{11, B} | |
| 857 | G857R⁶ | Lung |
| | | adenocarcinoma⁶ |
| 857, 851, | G857R/V851I/V845M/ | Lung |
| 845, 813, | Y813H/T785I⁶ | adenocarcinoma⁶ |
| and 785 | | |
| 858 | L858R¹ | |
| 858 and 108 | L858R/R108K^{11, B} | |
| 858 and 289 | L858R/A289T^{11, B} | |
| 858 and 292 | L858R/V292L^{11, B} | |
| 858 and 306 | L858R/S306L^{11, B} | |
| 858 and 703 | L858R/L703I^{11, B} | |
| 858 and 709 | L858R/E709A^{11, B}, | |
| | L858R/E709G^{11, B}, | |
| | L858R/E709K^{11, B}, | |
| | L858R/E709V^{11, B}, | |
| 858 and 714 | L858R/K714R^{11, B} | |
| 858 and 718 | L858R/L718Q¹ | |
| 858 and 720 | L858R/S720F^{11, B} | |
| 858 and 744 | L858R/I744M^{11, B} | |
| 858 and 768 | L858R/S768I^{11, B} | |
| 858 and 769 | L858R/V769L⁶ | Lung |
| | | adenocarcinoma⁶ |
| 858 and 776 | L858R/R776H⁶, L858R/R776C^{11, B}, | Lung |
| | L858R/R776G^{11, B} | adenocarcinoma⁶ |
| 858 and 790 | L858R/T790M^{11, B} | |
| 858 and 833 | L858R/L833V⁶ | Lung |
| | | adenocarcinoma⁶ |
| 858 and 838 | L858R/L838V^{11, B} | |
| 858 and 843 | L858R/V843I^{11, B} | |
| 858 and 844 | L858R/L844V¹ | |
| 858 and | L858R/del_189 | NSCLC⁹ |
| exon | | |
| 18 deletion | | |
| 859 | A859T^{11, B} | |
| 860 | K860R^{11, B} | |
| 861 | L861Q, L861R, L681G^{1, 5, 6, 7, 9} | Lung |
| | | adenocarcinoma⁶, |
| | | NSCLC^{5, 9} |
| 861 and 719 | L861Q/G719X¹⁰, | Lung adeno- |
| | L861Q/G719A^{11, B}, | carcinoma¹⁰ |
| | L861R/G719A^{11, B} | |
| 861 and 858 | L861Q/L858R^{11, B} | |
| 861, 768, | L861Q/S768I/G719X¹⁰ | Lung adeno- |
| and 719 | | carcinoma¹⁰ |
| 864 | A864T^{11, B} | |
| 865 | E865K^{11, B} | |
| 870 | H870R⁶ | Lung |
| | | adenocarcinoma⁶ |
| 870 and 858 | H870R/L858R | |
| 871 | A871E, A871G^{3, 6}, A871T^{11, B} | Lung |
| | | adenocarcinoma⁶ |
| 871 and 858 | A871G/L858R⁶ | Lung |
| | | adenocarcinoma⁶ |
| 873 | G873E^{11, B} | |
| 874 | G874S^{11, B} | |
| 941 | I941R¹ | |
| 948 | V948R¹ | |
| 1118 | A1118T^{11, B} | |
| 1153 | S1153I^{11, B} | |

Exon 19 insertion (sometimes also called ins_19)

| | |
|---|---|
| V738_K739insKIPVAI⁶ | Lung |
| | adenocarcinoma⁶ |
| I744_K745insKIPVAI^{9, 10} | NSCLC^{9, 10} |
| K745_E746insTPVAIK^{9, 10} | NSCLC^{9, 10} |
| K745_E746insVPVAIK¹⁰ | NSCLC¹⁰ |
| K745_E746insIPVAIK^{9, 10} | NSCLC^{9, 10} |

Exon 19 deletion (sometimes also called del_19)^{1, 2}

| | | |
|---|---|---|
| | E746_A750del¹ | |
| | E746_A750delinsP¹ | |
| | E746_A750delinsIP^{11, B} | |
| | E746_A750del/T790M^{11, B} | |
| | E746_A750del/A1118T^{11, B} | |
| | E746_T751delinsV^{11, B} | |
| | E746_S752delinsV² | Lung |
| | | adenocarcinoma² |
| | E746_S752delinsV/A216T^{11, B} | |
| | L747_E749del⁵ | NSCLC⁵ |
| | L747_A750del³ | NSCLC³ |
| | L747_A750delinsP² | Lung |
| | | adenocarcinoma² |
| | L747_T751del² | Lung |
| | | adenocarcinoma² |
| | L747_T751delinsA¹⁰ | NSCLC¹⁰ |
| | L747_T751delinsP¹⁰ | NSCLC¹⁰ |
| | L747_T751delinsS^{11, B} | |
| | L747_T751delinsQ² | Lung |
| | | adenocarcinoma² |
| | L747_S752del² | Lung |
| | | adenocarcinoma² |
| | L747_P753del¹⁰ | NSCLC¹⁰ |
| | L747_P753delinsS² | Lung |
| | | adenocarcinoma² |
| | L747_P753delinsQ^{11, B} | |
| | L747_P753delinsVS¹⁰ | NSCLC¹⁰ |
| | T751_I759delinsN^{11, B} | |
| | S752_I759del^{2, 10} | Lung |
| | | adenocarcinoma², |
| | | NSCLC¹⁰ |
| Exon 19 | Del_19 and I706T⁶ | Lung |
| deletion and | | adenocarcinoma⁶ |
| 706 | | |
| Exon 19 | Del_19 and L718Q¹ | |
| deletion and | | |
| 718 | | |
| Exon 19 | Del_19 and L844V¹ | |
| deletion and | | |
| 844 | | |
| Exon 19 | Del_19 and L858R⁶ | Lung |
| deletion and | | adenocarcinoma⁶ |
| 858 | | |
| Exon 19 | Del_19 and del_18⁹ | NSCLC⁹ |
| deletion and | | |
| Exon 18 | | |
| deletion | | |

Exon 20 insertion (sometimes also called ins_20)¹

| | | |
|---|---|---|
| | D761_E762insX⁵, e.g., | NSCLC⁵ |
| | D761_E762insEAFQ¹⁰ | |
| | A763_Y764insX⁵, e.g., | NSCLC^{5, 9, 10} |
| | A763_Y764insFQEA^{5, 9, 10} | |
| | Y764_V765insX⁵, e.g., | NSCLC⁵ |
| | V764_V765insHH¹³ | |
| | V765_M766insX⁵ | NSCLC⁵ |
| | M766_A767insASV⁶ | Lung |
| | | adenocarcinoma⁶ |
| | A767_S768insX⁵; A767insASV¹⁴ | NSCLC⁵ |
| | A767_V769dupASV⁵ | NSCLC⁵ |
| | S768_V769insX¹²; S768dupSVD¹⁴ | NSCLC |
| | V769_D770insX⁵, e.g., | NSCLC^{5, 8, 10} |
| | V769_D770insASV^{8, 9, 10} | |
| | D770delinsGY^{5, 10} | NSCLC^{5, 10} |
| | D770_N771insX⁵, e.g., | NSCLC^{3, 5, 10} |
| | D770_N771insNPG^{2, 5, 9}, | |
| | D770_N771insG + N771T⁶, | |
| | D770_N771insNPY⁶, | |
| | D770_N771insSVD^{9, 10}, | |
| | D770_N771insGL¹³ | |
| | D770_N771insX and | Lung |
| | amino acid position | adenocarcinoma⁶ |
| | 773, e.g., D770_N771insNPY/ | |
| | H773Y⁶ | |
| | N771_P772insX⁵, e.g., | NSCLC^{5, 10} |
| | N771_P772insN¹⁰, | |
| | N771_P772insH¹³, | |
| | N771_P772insV¹³ | |
| | N771_H773dupNPH⁵ | NSCLC⁵ |
| | N771delinsGY¹³; N771del insFH¹⁴ | |
| | P772_H773insX⁵, e.g., | NSCLC^{5, 9} |
| | P772_H773insDNP⁹, | |
| | P772_H773insPNP⁹ | |
| | H773_V774insX⁵, e.g., | NSCLC^{5, 9, 10} |
| | H773_V774insNPH⁹, | |
| | H773_V774insH¹⁰, | |
| | H773_V774insPH¹⁰, | |
| | H773_V774insAH¹⁰, | |
| | P772_H773insPNP¹³ | |
| | H773_dupH¹³ | |
| | V774_C775insX⁵, e.g., | NSCLC⁵ |
| | V774_C775insHV¹⁰ | |
| | A775_G776insX, e.g., | |
| | A775_G776insYVMA¹³ | |
| Kinase | Duplication of exons 18-25, | NSCLC⁹ |
| domain | 18-26, 14-26, | |
| duplication | or 17-25⁹ | |
| (KDD) | | |

^{A}The EGFR mutations shown may be activating mutations and/or confer increased resistance of EGFR to an EGFR inhibitor and/or a multi-kinase inhibitor (MKI), e.g., as compared to a wild type EGFR
^{B}Potentially oncogenic variant. See, e.g., Kohsaka, Shinji, et al. Science translational medicine 9.416 (2017): eaan6566.
¹PCT Patent Application Publication No. WO2019/246541.
²Grosse A, Grosse C, Rechsteiner M, Soltermann A. Diagn Pathol. 2019; 14(1): 18. Published 2019 Feb 11. doi: 10.1186/s13000-019-0789-1.
³Stewart EL, Tan SZ, Liu G, Tsao MS. Transl Lung Cancer Res. 2015; 4(1): 67-81. doi: 10.3978/j.issn.2218-6751.2014.11.06.
⁴Pines, Gur, Wolfgang J. Köstler, and Yosef Yarden. FEBS letters 584.12 (2010): 2699-2706.
⁵Yasuda, Hiroyuki, Susumu Kobayashi, and Daniel B. Costa. The Lancet Oncology 13.1 (2012): e23-e31.
⁶Kim EY, Cho EN, Park HS, et al. Cancer Biol Ther. 2016; 17(3): 237-245. doi: 10.1080/15384047.2016.1139235.
⁷Shah, Riyaz, and Jason F. Lester. Clinical Lung Cancer (2019).
⁸Aran, Veronica, and Jasminka Omerovic. International journal of molecular sciences 20.22 (2019): 5701. doi: 10.3390/ijms20225701.
⁹Beau-Faller, Michele, et al. (2012): 10507-10507. doi: 10.1016/j.semcancer.2019.09.015.
¹⁰Masood, Ashiq, Rama Krishna Kancha, and Janakiraman Subramanian. Seminars in oncology. WB Saunders, 2019. doi: 10.1053/j.seminoncol.2019.08.004.
¹¹Kohsaka, Shinji, et al. Science translational medicine 9.416 (2017): eaan6566.
¹²Vyse and Huang et al. Signal Transduct Target Ther. 2019 Mar 8; 4: 5. doi: 10.1038/s41392-019-0038-9.
¹³PCT Patent Application Publication No. WO2019/046775.
¹⁴PCT Patent Application Publication No. WO 2018/094225.

TABLE 1b
EGFR Protein Amino Acid Substitutions/Insertions/Deletions^{A}

| | | Non-limiting |
|---|---|---|
| Amino Acid | Non-Limiting Exemplary | Exemplary EGFR- |
| Position(s) | Mutations | associated Cancer(s) |
| 62 | L62R^{11,B} | |
| 108 | R108K^{11,B} | |
| 216 | A216T^{11,B} | |
| 222 | R222C^{11,B} | |
| 252 | R252C^{11,B} | |
| 289 | A289D, A289T, A289V^{11,B} | |
| 292 | V2921^{11,B} | |
| 304 | H304Y^{11,B} | |
| 306 | S306L^{11,B} | |
| 492 | S492R^{11,B} | |
| 596 | P596L^{11,B} | |
| 598 | G598V^{11,B} | |
| 688 | L688F⁶ | Lung adenocarcinoma⁶ |
| 689 | V689L⁶ | Lung adenocarcinoma⁶ |
| 703 | L703I, L703P^{11,B} | |
| 706 | I706T⁶ | Lung adenocarcinoma⁶ |
| 709 | E709A, E709G, E709K, | Lung adenocarcinoma^{2,6}, |
| | E709H, E709V^{2,6,9} | NSCLC⁹ |
| 709-710 | E709_T710delinsD⁹ | NSCLC⁹ |
| (sometimes | | |
| also called | | |
| exon 18 | | |
| deletion or | | |
| del_18) | | |
| 714 | K714R^{11,B} | |
| 718 | L718Q^{1,11,B} | |
| 719 | G719S, G719C, | NSCLC³ |
| | G719A, G719D^{1,4} | |
| 719 and 706 | G719A/I706T⁶ | Lung adenocarcinoma⁶ |
| 719 and 709 | G719S/E709K⁶, | Lung adenocarcinoma⁶ |
| | G719S/E709A^{11,B}, | |
| | G719C/E709A^{11,B} | |
| 719 and exon | G719X/del_18⁹ | NSCLC⁹ |
| 18 deletion | | |
| 720 | S720F^{11,B} | |
| 735 | G735S^{11,B} | |
| 741 | P741L^{11,B} | |
| 744 | I744M^{11,B} | |
| 747 | L747S^{11,B} | |
| 749 | E749Q⁶ | Lung adenocarcinoma⁶ |
| 750 | A750P^{5,10} | NSCLC⁵, lung |
| | | adenocarcinoma¹⁰ |
| 750 and 749 | A750P/E749Q⁶ | |
| 751 | T7511^{11,B} | |
| 752 | S752F¹⁰ | Lung adenocarcinoma¹⁰ |
| 753 | P753S^{11,B} | |
| 765 | V765A³, V765M^{11,B} | NSCLC³ |
| 767 | A767V^{11,B} | |
| 768 | S768I^{2,9} | Lung adenocarcinoma², |
| | | NSCLC⁹ |
| 768 and 719 | S768I/G719A^{11,B}, | |
| | S768I/G719C^{11,B}, | |
| | S7681/G719S^{11,B} | |
| 769 | V769M³, V769L^{11,B} | |
| 771 | N771F⁶ | Lung adenocarcinoma⁶ |
| 773 | H773Y⁶, H773L^{11,B}, | Lung adenocarcinoma⁶ |
| | H773Y^{11,B} | |
| 774 | V774M, V774A^{2,3} | Lung adenocarcinoma², |
| | | NSCLC³ |
| 774 and 773 | V774M/H773L¹⁰ | NSCLC¹⁰ |
| 776 | R776H, R776C⁶, R776G^{11,B} | Lung adenocarcinoma⁶ |
| 776 and 719 | R776H/G719S⁶ | Lung adenocarcinoma⁶ |
| 779 | G779S^{11,B} | |
| 783 | T783A³ | NSCLC³ |
| 784 | S784P³ | |
| 785 | T785I⁶ | Lung adenocarcinoma⁶ |
| 786 | V786M^{11,B} | |
| 790 | T790M³ | NSCLC³ |
| 790 and 719 | T790M/G719A^{11,B} | |
| 790 and 948 | T790M/V948R¹ | |
| 797 and 790 | C797S/T790M^{11,B} | |
| 798 | P798H^{11,B} | |
| 802 | V8021^{11,B} | |
| 813 | Y813H⁶ | Lung adenocarcinoma⁶ |
| 824 | G824S⁶ | Lung adenocarcinoma⁶ |
| 824 and 688 | G824S/L688F⁶ | Lung adenocarcinoma⁶ |
| 831 | R831H, R831L^{11,B} | |
| 833 | L833V^{2,8}, L833F^{11,B} | Lung adenocarcinoma², |
| | | NSCLC⁸ |
| 834 | V834L, V834M^{11,B} | |
| 835 | H835L^{11,B} | |
| 835, 833, | H835L/L833V/R670W⁸ | NSCLC⁸ |
| and 670 | | |
| 838 | L838V^{11,B} | |
| 843 | V843I³ | NSCLC³ |
| 844 | L844V¹ | |
| 845 | V845M⁶ | Lung adenocarcinoma⁶ |
| 848 | P848L^{11,B} | |
| 851 | V851I⁶ | Lung adenocarcinoma⁶ |
| 854 | T854A^{11,B} | |
| 857 | G857R⁶ | Lung adenocarcinoma⁶ |
| 857, 851, | G857R/V851I/V845M/ | Lung adenocarcinoma⁶ |
| 845, 813, | Y813H/T785I⁶ | |
| and 785 | | |
| 858 | L858R¹ | |
| 858 and 108 | L858R/R108K^{11,B} | |
| 858 and 289 | L858R/A289T^{11,B} | |
| 858 and 292 | L858R/V292L^{11,B} | |
| 858 and 306 | L858R/S306L^{11,B} | |
| 858 and 703 | L858R/L7031^{11,B} | |
| 858 and 709 | L858R/E709A^{11,B}, | |
| | L858R/E709G^{11,B}, | |
| | L858R/E709K^{11,B}, | |
| | L858R/E709V^{11,B}, | |
| 858 and 714 | L858R/K714R^{11,B} | |
| 858 and 718 | L858R/L718Q¹ | |
| 858 and 720 | L858R/S720F^{11,B} | |
| 858 and 744 | L858R/I744M^{11,B} | |
| 858 and 768 | L858R/S7681^{11,B} | |
| 858 and 769 | L858R/V769L⁶ | Lung adenocarcinoma⁶ |
| 858 and 776 | L858R/R776H⁶, | Lung adenocarcinoma⁶ |
| | L858R/R776C^{11,B}, | |
| | L858R/R776G^{11,B} | |
| 858 and 790 | L858R/T790M^{11,B} | |
| 858 and 833 | L858R/L833V⁶ | Lung adenocarcinoma⁶ |
| 858 and 838 | L858R/L838V^{11,B} | |
| 858 and 843 | L858R/V843I^{11,B} | |
| 858 and 844 | L858R/L844V¹ | |
| 858 and exon | L858R/del_189 | NSCLC⁹ |
| 18 deletion | | |
| 859 | A859T^{11,B} | |
| 860 | K860R^{11,B} | |
| 861 | L861Q, L861R, | Lung adenocarcinoma⁶, |
| | L681G^{1,5,6,7,9} | NSCLC^{5,9} |
| 861 and 719 | L861Q/G719X¹⁰, | Lung adenocarcinoma¹⁰ |
| | L861Q/G719A^{11,B}, | |
| | L861R/G719A^{11,B} | |
| 861 and 858 | L861Q/L858R^{11,B} | |
| 861, 768, | L861Q/S768I/G719X¹⁰ | Lung adenocarcinoma¹⁰ |
| and 719 | | |
| 864 | A864T^{11,B} | |
| 865 | E865K^{11,B} | |
| 870 | H870R⁶ | Lung adenocarcinoma⁶ |
| 870 and 858 | H870R/L858R | |
| 871 | A871E, A871G^{3,6}, | Lung adenocarcinoma⁶ |
| | A871T^{11,B} | |
| 871 and 858 | A871G/L858R⁶ | Lung adenocarcinoma⁶ |
| 873 | G873E^{11,B} | |
| 874 | G874S^{11,B} | |
| 941 | I941R¹ | |
| 948 | V948R¹ | |
| 1118 | A1118T^{11,B} | |
| 1153 | S1153I^{11,B} | |

Exon 19 insertion (sometimes also called ins_19)

| | |
|---|---|
| V738_K739insKIPVAI⁶ | Lung adenocarcinoma⁶ |
| I744_K745insKIPVAI^{9,10} | NSCLC^{9,10} |
| K745_E746insTPVAIK^{9,10} | NSCLC^{9,10} |
| K745_E746insVPVAIK¹⁰ | NSCLC¹⁰ |
| K745_E746insIPVAIK^{9,10} | NSCLC^{9,10} |

Exon 19 deletion (sometimes also called del_19)^{1,2}

| | | |
|---|---|---|
| | E746_A750del¹ | |
| | E746_A750delinsP¹ | |
| | E746_A750delinsIP^{11,B} | |
| | E746_A750del/T790M^{11,B} | |
| | E746_A750del/A1118T^{11,B} | |
| | E746_T751delinsV^{11,B} | |
| | E746_S752delinsV² | Lung adenocarcinoma² |
| | E746_S752delinsV/ | |
| | A216T^{11,B} | |
| | L747_E749del⁵ | NSCLC⁵ |
| | L747_A750del³ | NSCLC³ |
| | L747_A750delinsP² | Lung adenocarcinoma² |
| | L747_T751del² | Lung adenocarcinoma² |
| | L747_T751delinsA¹⁰ | NSCLC¹⁰ |
| | L747_T751delinsP¹⁰ | NSCLC¹⁰ |
| | L747_T751delinsS^{11,B} | |
| | L747_T751delinsQ² | Lung adenocarcinoma² |
| | L747_S752del² | Lung adenocarcinoma² |
| | L747_P753del¹⁰ | NSCLC¹⁰ |
| | L747_P753delinsS² | Lung adenocarcinoma² |
| | L747_P753delinsQ^{11,B} | |
| | L747_P753delinsVS¹⁰ | NSCLC¹⁰ |
| | T751_I759delinsN^{11,B} | |
| | S752_I759del^{2,10} | Lung adenocarcinoma², |
| | | NSCLC¹⁰ |
| Exon 19 | Del_19 and I706T⁶ | Lung adenocarcinoma6 |
| deletion and | | |
| 706 | | |
| Exon 19 | Del_19 and L718Q¹ | |
| deletion and | | |
| 718 | | |
| Exon 19 | Del_19 and L844V¹ | |
| deletion and | | |
| 844 | | |
| Exon 19 | Del_19 and L858R⁶ | Lung adenocarcinoma⁶ |
| deletion and | | |
| 858 | | |
| Exon 19 | Del_19 and del_18⁹ | NSCLC⁹ |
| deletion and | | |
| Exon 18 | | |
| deletion | | |

Exon 20 insertion (sometimes also called ins_20)¹

| | | |
|---|---|---|
| | D761_E762insX⁵, e.g., | NSCLC⁵ |
| | D761_E762insEAFQ¹⁰ | |
| | A763_Y764insX⁵, e.g., | NSCLC^{5,9,10} |
| | A763_Y764insFQEA^{5,9,10} | |
| | Y764_V765insX⁵, e.g., | NSCLC⁵ |
| | V764_V765insHH¹³ | |
| | V765_M766insX⁵ | NSCLC⁵ |
| | M766_A767insASV⁶ | Lung adenocarcinoma⁶ |
| | M766delinsMASVx2¹⁵ | Pediatric bithalamic |
| | | glioma¹⁵ |
| | A767_S768insX⁵; | NSCLC⁵ |
| | A767insASV¹⁴ | |
| | A767_V769dupASV⁵ | NSCLC⁵ |
| | A767delinsASVDx3; | Pediatric bithalamic |
| | A767delinsASVG¹⁵ | glioma¹⁵ |
| | S768_V769insX¹²; | NSCLC^{12,14}, Sinonasal |
| | S768dupSVD^{14,16} | squamous cell |
| | | carcinoma¹⁶ |
| | V769_D770insX⁵, e.g., | NSCLC^{5,8,10} |
| | V769_D770insASV^{8,9,10} | |
| | D770delinsGY^{5,10}; | NSCLC^{5,10}, Pediatric |
| | D770delinsDN; | bithalamic glioma¹⁵ |
| | D770delinsDNPH¹⁵ | |
| | D770_N771insX^{5,16}, e.g., | NSCLC^{3,5,10}, Sinonasal |
| | D770_N771insNPG^{2,5,9}, | squamous cell |
| | D770_N771insG + N771T⁶, | carcinoma¹⁶ |
| | D770_N771insNPY⁶, | |
| | D770_N771insSVD^{9,10}, | |
| | D770_N771insGL¹³ | |
| | D770_N771insX and amino | Lung adenocarcinoma⁶ |
| | acid position 773, e.g., | |
| | D770_N771insNPY/H773Y⁶ | |
| | N771_P772insX^{5,16}, e.g., | NSCLC^{5,10}; Sinonasal |
| | N771_P772insN¹⁰, | squamous cell |
| | N771_P772insH¹³, | carcinoma¹⁶ |
| | N771_P772ins V¹³ | |
| | N771_H773dupNPH⁵ | NSCLC⁵ |
| | N771delinsGY¹³; | NSCLC^{13,14}, Pediatric |
| | N771del insFH¹⁴; | bithalamic glioma¹⁵ |
| | N771_delinsNPH¹⁵ | |
| | N771_H773dup¹⁶ | Sinonasal squamous |
| | | cell carcinoma¹⁶ |
| | P772_H773insX⁵, e.g., | NSCLC^{5,9} |
| | P772_H773insDNP⁹, | |
| | P772_H773insPNP⁹ | |
| | H773_V774insX⁵, e.g., | NSCLC^{5,9,10} |
| | H773_V774insNPH⁹, | |
| | H773_V774insH¹⁰, | |
| | H773_V774insPH¹⁰, | |
| | H773_V774insAH¹⁰, | |
| | P772_H773insPNP¹³ | |
| | H773_dupH¹³ | |
| | V774_C775insX⁵, e.g., | NSCLC⁵ |
| | V774_C775insHV¹⁰ | |
| | A775_G776insX, e.g., | |
| | A775_G776insYVMA¹³ | |
| Kinase | Duplication of exons 18-25, | NSCLC⁹ |
| domain | 18-26, 14-26, or 17-25⁹ | |
| duplication | | |
| (KDD) | | |

^{A}The EGFR mutations shown may be activating mutations and/or confer increased resistance of EGFR to an EGFR inhibitor and/or a multi-kinase inhibitor (MKI), e.g., as compared to a wild type EGFR
^{B}Potentially oncogenic variant. See, e.g., Kohsaka, Shinji, et al. Science translational medicine 9.416 (2017): eaan6566.
¹PCT Patent Application Publication No. WO2019/246541.
²Grosse A, Grosse C, Rechsteiner M, Soltermann A. Diagn Pathol. 2019;14(1):18. Published 2019 Feb. 11. doi: 10.1186/s13000-019-0789-1.
³Stewart EL, Tan SZ, Liu G, Tsao MS. Transl Lung Cancer Res. 2015;4(1):67-81. doi: 10.3978/j.issn.2218-6751.2014.11.06.
⁴Pines, Gur, Wolfgang J. Köstler, and Yosef Yarden. FEBS letters 584.12 (2010): 2699-2706.
⁵Yasuda, Hiroyuki, Susumu Kobayashi, and Daniel B. Costa. The Lancet Oncology 13.1 (2012): e23-e31.
⁶Kim EY, Cho EN, Park HS, et al. Cancer Biol Ther. 2016;17(3):237-245. doi: 10.1080/15384047.2016.1139235.
⁷Shah, Riyaz, and Jason F. Lester. Clinical Lung Cancer (2019).
⁸Aran, Veronica, and Jasminka Omerovic. International journal of molecular sciences 20.22 (2019): 5701. doi: 10.3390/ijms20225701.
⁹Beau-Faller, Michele, et al. (2012): 10507-10507. doi: 10.1016/j.semcancer.2019.09.015.
¹⁰Masood, Ashiq, Rama Krishna Kancha, and Janakiraman Subramanian. Seminars in oncology. WB Saunders, 2019. doi: 10.1053/j.seminoncol.2019.08.004.
¹¹Kohsaka, Shinji, et al. Science translational medicine 9.416 (2017): eaan6566.
¹²Vyse and Huang et al. Signal Transduct Target Ther. 2019 Mar. 8;4:5. doi: 10.1038/s41392-019-0038-9.
¹³PCT Patent Application Publication No. WO2019/046775.
¹⁴PCT Patent Application Publication No. WO 2018/094225.
¹⁵Mondal, Gourish, et al. Acta Neuropathol. 2020; 139(6): 1071-1088
¹⁶Udager, Aaron M., et al. Cancer Res, 2015; 75(13): 2600-2606

In some embodiments, the dysregulation of an EGFR gene, an EGFR kinase, or expression or activity or level of any of the same, includes a splice variation in an EGFR mRNA which results in an expressed protein that is an alternatively spliced variant of EGFR having at least one residue deleted (as compared to the wild type EGFR kinase) resulting in a constitutive activity of an EGFR kinase domain.

In some embodiments, the dysregulation of an EGFR gene, an EGFR kinase, or expression or activity or level of any of the same, includes at least one point mutation in an EGFR gene that results in the production of an EGFR kinase that has one or more amino acid substitutions or insertions or deletions in an EGFR gene that results in the production of an EGFR kinase that has one or more amino acids inserted or removed, as compared to the wild type EGFR kinase. In some cases, the resulting EGFR kinase is more resistant to inhibition (e.g., inhibition of its signaling activity) by one or more first EGFR inhibitors, as compared to a wild type EGFR kinase or an EGFR kinase not including the same mutation. Such mutations, optionally, do not decrease the sensitivity of the cancer cell or tumor having the EGFR kinase to treatment with a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, (e.g., as compared to a cancer cell or a tumor that does not include the particular EGFR inhibitor resistance mutation).

In other embodiments, the dysregulation of an EGFR gene, an EGFR kinase, or expression or activity or level of any of the same, includes at least one point mutation in an EGFR gene that results in the production of an EGFR kinase that has one or more amino acid substitutions as compared to the wild type EGFR kinase, and which has increased resistance to a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as compared to a wild type EGFR kinase or an EGFR kinase not including the same mutation. In such embodiments, an EGFR inhibitor resistance mutation can result in an EGFR kinase that has one or more of an increased Vₘₐₓ, a decreased Kₘ, and a decreased K_{D} in the presence of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as compared to a wild type EGFR kinase or an EGFR kinase not having the same mutation in the presence of the same compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof.

Exemplary Sequence of Mature Human EGFR Protein (UniProtKB entry P00533) (SEQ ID NO: 1)

MRPSGTAGAA LLALLAALCP ASRALEEKKV
CQGTSNKLTQ LGTFEDHFLS
LQRMENNCEV VLGNLEITYV QRNYDLSFLK
TIQEVAGYVL IALNTVERIP
LENLQIIRGN MYYENSYALA VLSNYDANKT
GLKELPMRNL QEILHGAVRF
SNNPALCNVE SIQWRDIVSS DFLSNMSMDF
QNHLGSCQKC DPSCPNGSCW
GAGEENCQKL TKIICAQQCS GRCRGKSPSD
CCHNQCAAGC TGPRESDCLV
CRKFRDEATC KDTCPPLMLY NPTTYQMDVN
PEGKYSFGAT CVKKCPRNYV
VTDHGSCVRA CGADSYEMEE DGVRKCKKCE
GPCRKVCNGI GIGEFKDSLS
INATNIKHFK NCTSISGDLH ILPVAFRGDS
FTHTPPLDPQ ELDILKTVKE
ITGFLLIQAW PENRTDLHAF ENLEIIRGRT
KQHGQFSLAV VSLNITSLGL
RSLKEISDGD VIISGNKNLC YANTINWKKL
FGTSGQKTKI ISNRGENSCK
ATGQVCHALC SPEGCWGPEP RDCVSCRNVS
RGRECVDKCN LLEGEPREFV
ENSECIQCHP ECLPQAMNIT CTGRGPDNCI
QCAHYIDGPH CVKTCPAGVM
GENNTLVWKY ADAGHVCHLC HPNCTYGCTG
PGLEGCPTNG PKIPSIATGM
VGALLLLLVV ALGIGLFMRR RHIVRKRTLR
RLLQERELVE PLTPSGEAPN
QALLRILKET EFKKIKVLGS GAFGTVYKGL
WIPEGEKVKI PVAIKELREA
TSPKANKEIL DEAYVMASVD NPHVCRLLGI
CLTSTVQLIT QLMPFGCLLD
YVREHKDNIG SQYLLNWCVQ IAKGMNYLED
RRLVHRDLAA RNVLVKTPQH
VKITDFGLAK LLGAEEKEYH AEGGKVPIKW
MALESILHRI YTHQSDVWSY
GVTVWELMTF GSKPYDGIPA SEISSILEKG
ERLPQPPICT IDVYMIMVKC
WMIDADSRPK FRELIIEFSK MARDPQRYLV
IQGDERMHLP SPTDSNFYRA
LMDEEDMDDV VDADEYLIPQ QGFFSSPSTS
RTPLLSSLSA TSNNSTVACI
DRNGLQSCPI KEDSFLQRYS SDPTGALTED
SIDDTFLPVP EYINQSVPKR
PAGSVQNPVY HNQPLNPAPS RDPHYQDPHS
TAVGNPEYLN TVQPTCVNST
FDSPAHWAQK GSHQISLDNP DYQQDFFPKE
AKPNGIFKGS TAENAEYLRV
APQSSEFIGA

In some embodiments, dysregulation of an EGFR gene, an EGFR kinase, or expression or activity or level of any of the same, includes at least one EGFR inhibitor resistance mutation in an EGFR gene that results in the production of an EGFR kinase that has one or more of the amino acid substitutions, insertions, or deletions as described in Table 2a and Table 2b. In some embodiments, compounds of Formula (I) (e.g., Formula (I-a), (I-b), (I-c), (I-d), (I-e), (I-f), (I-g), (I-h), (I-i), (I-j), or (I-k)) and pharmaceutically acceptable salts and solvates thereof, are useful in treating subjects that develop cancers with EGFR inhibitor resistance mutations (e.g., that result in an increased resistance to a first EGFR inhibitor, e.g., a substitution at amino acid position 718, 747, 761, 790, 797, or 854 (e.g., L718Q, L747S, D761Y, T790M, C797S, T854A), and/or one or more EGFR inhibitor resistance mutations listed in Table 2a and Table 2b) by either dosing in combination or as a subsequent or additional (e.g., follow-up) therapy to existing drug treatments (e.g., other inhibitors of EGFR; e.g., first and/or second EGFR inhibitors).

TABLE 2a
EGFR Protein Amino Acid Resistance Mutations

| | | Non-limiting |
|---|---|---|
| | | Exemplary |
| | | EGFR- |
| Amino Acid | Non-Limiting Exemplary | associated |
| Position(s) | Mutations | Cancer(s) |
| 747 | L747S^{2,4,6}, L747P⁶ | NSCLC² |
| 761 | D761Y^{2,4} | NSCLC² |
| 769 | V769M² | NSCLC² |
| 790 | T790M^{1,4} | NSCLC² |
| 792 | L792H⁶ | NSCLC⁶ |
| 796 | G796R⁶ | NSCLC⁶ |
| 797 | C797S⁵ | NSCLC⁵ |
| 797 and 790 | C797S/T790M⁵ | NSCLC⁵ |
| 843 | V843I^{2,5} | NSCLC² |
| 854 | T854A^{2,4} | NSCLC² |
| 858 and 747 | L858R/L747S⁶ | NSCLC⁶ |
| 858 and 790 | L858R/T790M¹ | |
| 858, 797, and | L858R/C797S/T790M¹ | |
| 790 | | |
| 871 | A871E² | NSCLC² |
| 941, 858, and | I941R/L858R/T790M¹ | |
| 790 | | |
| Exon 19 deletion | Del_19 and T790M¹ | |
| and 790 | | |
| Exon 19 | Del_19 and T790M/L844V¹ | |
| deletion, 844, | | |
| and 790 | | |
| Exon 19 | Del_19 and C797S/T790M¹ | |
| deletion, 797, | | |
| and 790 | | |

Exon 20 insertion (also called ins_20)^{2,3}

| | |
|---|---|
| A767_V769dupASV⁵ | NSCLC⁵ |
| D770 N771insX³, e.g., | NSCLC^{2,3} |
| D770 N771insNPG^{2,3,7} | |
| N771_H771dupNPH³ | NSCLC³ |
| P772_H773insX³ e.g., | NSCLC^{3,7} |
| P772_H773insDNP⁷ | |
| H773_V774insNPH⁷ | NSCLC⁷ |

¹PCT Patent Application Publication No. WO2019/246541
²Stewart EL, Tan SZ, Liu G, Tsao MS. Transl Lung Cancer Res. 2015;4(1):67-81. doi: 10.3978/j.issn.2218-6751.2014.11.06
³Yasuda, Hiroyuki, Susumu Kobayashi, and Daniel B. Costa. The Lancet Oncology 13.1 (2012): e23-e31.
⁴Kim EY, Cho EN, Park HS, et al. Cancer Biol Ther. 2016;17(3):237-245. doi: 10.1080/15384047.2016.1139235
⁵Shah, Riyaz, and Jason F. Lester. Clinical Lung Cancer (2019).
⁶Aran, Veronica, and Jasminka Omerovic. International journal of molecular sciences 20.22 (2019): 5701. doi: 10.3390/ijms20225701.
⁷Beau-Faller, Michele, et al. (2012): 10507-10507. doi: 10.1016/j.semcancer.2019.09.015
⁸Masood, Ashiq, Rama Krishna Kancha, and Janakiraman Subramanian. Seminars in oncology. WB Saunders, 2019. doi: 10.1053/j.seminoncol.2019.08.004

TABLE 2b
EGFR Protein Amino Acid Resistance Mutations

| | | Non-limiting |
|---|---|---|
| | | Exemplary |
| | | EGFR- |
| Amino Acid | Non-Limiting Exemplary | associated |
| Position(s) | Mutations | Cancer(s) |
| 747 | L747S^{2,4,6}, L747P⁶ | NSCLC² |
| 761 | D761Y^{2,4} | NSCLC² |
| 769 | V769M² | NSCLC² |
| 790 | T790M^{1,4} | NSCLC² |
| 792 | L792H⁶ | NSCLC⁶ |
| 796 | G796R⁶ | NSCLC⁶ |
| 797 | C797S⁵ | NSCLC⁵ |
| 797 and 790 | C797S/T790M⁵ | NSCLC⁵ |
| 843 | V843I^{2,5} | NSCLC² |
| 854 | T854A^{2,4} | NSCLC² |
| 858 and 747 | L858R/L747S⁶ | NSCLC⁶ |
| 858 and 790 | L858R/T790M¹ | |
| 858 and 797 | L858R/C797S (or C797G)⁹ | NSCLC⁹ |
| 858, 797, and | L858R/C797S/T790M¹ | |
| 790 | | |
| 871 | A871E² | NSCLC² |
| 941, 858, and | I941R/L858R/T790M¹ | |
| 790 | | |
| Exon 19 | Del_19 and T790M¹ | |
| deletion and | | |
| 790 | | |
| Exon 19 | Del_19 and C797S (or C797G)⁹ | NSCLC⁹ |
| deletion and | | |
| 797 | | |
| Exon 19 | Del_19 and T790M/L844V¹ | |
| deletion, 844, | | |
| and 790 | | |
| Exon 19 | Del_19 and C797S/T790M¹ | |
| deletion, 797, | | |
| and 790 | | |

Exon 20 insertion (also called ins_20)^{2,3}

| | |
|---|---|
| A767_V769dupASV⁵ | NSCLC⁵ |
| D770_N771insX³, e.g., | NSCLC^{2,3} |
| D770_N771insNPG^{2,3,7} | |
| N771_H771dupNPH³ | NSCLC³ |
| P772_H773insX³ e.g., | NSCLC^{3,7} |
| P772_H773insDNP⁷ | |
| H773_V774insNPH⁷ | NSCLC⁷ |

¹PCT Patent Application Publication No. WO2019/246541
²Stewart EL, Tan SZ, Liu G, Tsao MS. Transl Lung Cancer Res. 2015;4(1):67-81. doi: 10.3978/j.issn.2218-6751.2014.11.06
³Yasuda, Hiroyuki, Susumu Kobayashi, and Daniel B. Costa. The Lancet Oncology 13.1 (2012): e23-e31.
⁴Kim EY, Cho EN, Park HS, et al. Cancer Biol Ther. 2016;17(3):237-245. doi: 10.1080/15384047.2016.1139235
⁵Shah, Riyaz, and Jason F. Lester. Clinical Lung Cancer (2019).
⁶Aran, Veronica, and Jasminka Omerovic. International journal of molecular sciences 20.22 (2019): 5701. doi: 10.3390/ijms20225701.
⁷Beau-Faller, Michele, et al. (2012): 10507-10507. doi: 10.1016/j.semcancer.2019.09.015
⁸Masood, Ashiq, Rama Krishna Kancha, and Janakiraman Subramanian. Seminars in oncology. WB Saunders, 2019. doi: 10.1053/j.seminoncol.2019.08.004
⁹Papadimitrakopoulou, V.A., et al. Annals of Oncology 2018; 29 Supplement 8 VIII741

In some embodiments, the EGFR Protein Amino Acid Substitutions/Insertions/Deletions include any one or more, or any two or more (e.g., any two), of the EGFR Protein Amino Acid Substitutions/Insertions/Deletions delineated in Table 1a, 1b and/or Table 2a, 2b; e.g., any one or more, or any two or more (e.g., any two), of the following and independently selected EGFR Protein Amino Acid Substitutions/Insertions/Deletions: V769L; V769M; M766delinsMASVx2; A767_V769dupASV; A767delinsASVDx3; A767delinsASVG; S768_V769insX; V769_D770insX; V769_D770insASV; D770delinsDN; D770delinsDNPH; D770_N771insSV; N771delinsNPH; N771_H773dup; L858R/C797S (or C797G); or Del_19 and C797S (or C797G), or any combination thereof.

As used herein, a “first inhibitor of EGFR” or “first EGFR inhibitor” is an EGFR inhibitor as defined herein, but which does not include a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as defined herein. As used herein, a “second inhibitor of EGFR” or a “second EGFR inhibitor” is an EGFR inhibitor as defined herein, but which does not include a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as defined herein. When both a first and a second inhibitor of EGFR are present in a method provided herein, the first and second inhibitors of EGFR are different. In some embodiments, the first and/or second inhibitor of EGFR bind in a different location than a compound of Formula (I) (e.g., Formula (I-a), (I-b), (I-c), (I-d), (I-e), (I-f), (I-g), (I-h), (I-i), (I-j), or (I-k)). For example, in some embodiments, a first and/or second inhibitor of EGFR can inhibit dimerization of EGFR, while a compound of Formula (I) (e.g., Formula (I-a), (I-b), (I-c), (I-d), (I-e), (I-f), (I-g), (I-h), (I-i), (I-j), or (I-k)) can inhibit the active site. In some embodiments, a first and/or second EGFR inhibitor can be an allosteric inhibitor of EGFR, while a compound of Formula (I) (e.g., Formula (I-a), (I-b), (I-c), (I-d), (I-e), (I-f), (I-g), (I-h), (I-i), (I-j), or (I-k)) can inhibit the EGFR active site.

Exemplary first and second inhibitors of EGFR are described herein. In some embodiments, a first or second inhibitor of EGFR can be selected from the group consisting of osimertinib, gefitinib, erlotinib, afatinib, lapatinib, neratinib, AZD-9291, CL-387785, CO-1686, or WZ4002.

In some embodiments, compounds of Formula (I) (e.g., Formula (I-a), (I-b), (I-c), (I-d), (I-e), (I-f), (I-g), (I-h), (I-i), (I-j), or (I-k)), or pharmaceutically acceptable salts and solvates thereof, are useful for treating a cancer that has been identified as having one or more EGFR inhibitor resistance mutations (that result in an increased resistance to a first or second inhibitor of EGFR, e.g., a substitution described in Table 2a and Table 2b including substitutions at amino acid position 747, 761, 790, 797, or 854 (e.g., L718Q, L747S, D761Y, T790M, C797S, T854A)). In some embodiments, the one or more EGFR inhibitor resistance mutations occurs in a nucleic acid sequence encoding a mutant EGFR protein (e.g., a mutant EGFR protein having any of the mutations described in Table 2a and Table 2b) resulting in a mutant EGFR protein that exhibits EGFR inhibitor resistance.

The epidermal growth factor receptor (EGFR) belongs to the ErbB family of receptor tyrosine kinases (RTKs) and provides critical functions in epithelial cell physiology (Schlessinger J (2014) Cold Spring Harb Perspect Biol 6, a008912). It is frequently mutated and/or overexpressed in different types of human cancers and is the target of multiple cancer therapies currently adopted in the clinical practice (Yarden Y and Pines G (2012) Nat Rev Cancer 12, 553-563).

Accordingly, provided herein are methods for treating a subject diagnosed with (or identified as having) a cancer that include administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof.

Also provided herein are methods for treating a subject identified or diagnosed as having an EGFR-associated cancer that include administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. In some embodiments, the subject that has been identified or diagnosed as having an EGFR-associated cancer through the use of a regulatory agency-approved, e.g., FDA-approved test or assay for identifying dysregulation of an EGFR gene, an EGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is an EGFR-associated cancer. For example, the EGFR-associated cancer can be a cancer that includes one or more EGFR inhibitor resistance mutations.

The term “regulatory agency” refers to a country's agency for the approval of the medical use of pharmaceutical agents with the country. For example, a non-limiting example of a regulatory agency is the U.S. Food and Drug Administration (FDA).

Also provided are methods for treating cancer in a subject in need thereof, the method comprising: (a) detecting an EGFR-associated cancer in the subject; and (b) administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. Some embodiments of these methods further include administering to the subject another anticancer agent (e.g., a second EGFR inhibitor, a second compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or an immunotherapy). In some embodiments, the subject was previously treated with a first EGFR inhibitor or previously treated with another anticancer treatment, e.g., at least partial resection of the tumor or radiation therapy. In some embodiments, the subject is determined to have an EGFR-associated cancer through the use of a regulatory agency-approved, e.g., FDA-approved test or assay for identifying dysregulation of an EGFR gene, an EGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is an EGFR-associated cancer. For example, the EGFR-associated cancer can be a cancer that includes one or more EGFR inhibitor resistance mutations.

Also provided are methods of treating a subject that include performing an assay on a sample obtained from the subject to determine whether the subject has a dysregulation of an EGFR gene, an EGFR kinase, or expression or activity or level of any of the same, and administering (e.g., specifically or selectively administering) a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof, to the subject determined to have a dysregulation of an EGFR gene, an EGFR kinase, or expression or activity or level of any of the same. Some embodiments of these methods further include administering to the subject another anticancer agent (e.g., a second EGFR inhibitor, a second compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or immunotherapy). In some embodiments of these methods, the subject was previously treated with a first EGFR inhibitor or previously treated with another anticancer treatment, e.g., at least partial resection of a tumor or radiation therapy. In some embodiments, the subject is a subject suspected of having an EGFR-associated cancer, a subject presenting with one or more symptoms of an EGFR-associated cancer, or a subject having an elevated risk of developing an EGFR-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art. In some embodiments, the dysregulation of an EGFR gene, an EGFR kinase, or expression or activity or level of any of the same includes one or more EGFR inhibitor resistance mutations.

Also provided is a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof, for use in treating an EGFR-associated cancer in a subject identified or diagnosed as having an EGFR-associated cancer through a step of performing an assay (e.g., an in vitro assay) on a sample obtained from the subject to determine whether the subject has a dysregulation of an EGFR gene, an EGFR kinase, or expression or activity or level of any of the same, where the presence of a dysregulation of an EGFR gene, an EGFR kinase, or expression or activity or level of any of the same, identifies that the subject has an EGFR-associated cancer. Also provided is the use of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for the manufacture of a medicament for treating an EGFR-associated cancer in a subject identified or diagnosed as having an EGFR-associated cancer through a step of performing an assay on a sample obtained from the subject to determine whether the subject has a dysregulation of an EGFR gene, an EGFR kinase, or expression or activity or level of any of the same where the presence of dysregulation of an EGFR gene, an EGFR kinase, or expression or activity or level of any of the same, identifies that the subject has an EGFR-associated cancer. Some embodiments of any of the methods or uses described herein further include recording in the subject's clinical record (e.g., a computer readable medium) that the subject is determined to have a dysregulation of an EGFR gene, an EGFR kinase, or expression or activity or level of any of the same, through the performance of the assay, should be administered a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. In some embodiments, the dysregulation of an EGFR gene, an EGFR kinase, or expression or activity or level of any of the same includes one or more EGFR inhibitor resistance mutations.

Also provided is a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for use in the treatment of a cancer in a subject in need thereof, or a subject identified or diagnosed as having an EGFR-associated cancer. Also provided is the use of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for the manufacture of a medicament for treating a cancer in a subject identified or diagnosed as having an EGFR-associated cancer. In some embodiments, the cancer is an EGFR-associated cancer, for example, an EGFR-associated cancer having one or more EGFR inhibitor resistance mutations. In some embodiments, a subject is identified or diagnosed as having an EGFR-associated cancer through the use of a regulatory agency-approved, e.g., FDA-approved, kit for identifying dysregulation of an EGFR gene, an EGFR kinase, or expression or activity or level of any of the same, in a subject or a biopsy sample from the subject. As provided herein, an EGFR-associated cancer includes those described herein and known in the art.

In some embodiments of any of the methods or uses described herein, the subject has been identified or diagnosed as having a cancer with a dysregulation of an EGFR gene, an EGFR kinase, or expression or activity or level of any of the same. In some embodiments of any of the methods or uses described herein, the subject has a tumor that is positive for a dysregulation of an EGFR gene, an EGFR kinase, or expression or activity or level of any of the same. In some embodiments of any of the methods or uses described herein, the subject can be a subject with a tumor(s) that is positive for a dysregulation of an EGFR gene, an EGFR kinase, or expression or activity or level of any of the same. In some embodiments of any of the methods or uses described herein, the subject can be a subject whose tumors have a dysregulation of an EGFR gene, an EGFR kinase, or expression or activity or level of any of the same. In some embodiments of any of the methods or uses described herein, the subject is suspected of having an EGFR-associated cancer (e.g., a cancer having one or more EGFR inhibitor resistance mutations). In some embodiments, provided herein are methods for treating an EGFR-associated cancer in a subject in need of such treatment, the method comprising a) detecting a dysregulation of an EGFR gene, an EGFR kinase, or the expression or activity or level of any of the same in a sample from the subject; and b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the dysregulation of an EGFR gene, an EGFR kinase, or the expression or activity or level of any of the same includes one or more EGFR kinase protein point mutations/insertions/deletions. Non-limiting examples of EGFR kinase protein point mutations/insertions/deletions are described in Table 1a and Table 1b. In some embodiments, the EGFR kinase protein point mutations/insertions/deletions are selected from the group consisting of G719S, G719C, G719A, L747S, D761Y, T790M, T854A, L858R, L861Q, a deletion in exon 19 (e.g., L747_A750del), and an insertion in exon 20. In some embodiments, the EGFR kinase protein point mutations/insertions/deletions are selected from the group consisting of L858R, deletions in exon 19 (e.g., L747_A750del), L747S, D761Y, T790M, and T854A. In some embodiments, the dysregulation of an EGFR gene, an EGFR kinase, or the expression or activity or level of any of the same includes one or more EGFR inhibitor resistance mutations. Non-limiting examples of EGFR inhibitor resistance mutations are described in Table 2a and Table 2b. In some embodiments, the EGFR inhibitor resistance mutation is a substitution at amino acid position 718, 747, 761, 790, 797, or 854 (e.g., L718Q, L747S, D761Y, T790M, C797S, and T854A). In some embodiments, the dysregulation of an EGFR gene, an EGFR kinase, or the expression or activity or level of any of the same includes one or more point mutations/insertions/deletions in exon 20. Non-limiting examples of EGFR exon 20 mutations are described in Tables 1a, 1b, 2a and 2b. In some embodiments, the EGFR exon 20 mutation is an exon 20 insertion such as V769_D770insX, D770_N771insX, N771_P772insX, P772_H773insX, and H773_V774insX. For example, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP. In some embodiments, the cancer with a dysregulation of an EGFR gene, an EGFR kinase, or expression or activity or level of any of the same is determined using a regulatory agency-approved, e.g., FDA-approved, assay or kit. In some embodiments, the tumor that is positive for a dysregulation of an EGFR gene, an EGFR kinase, or expression or activity or level of any of the same is a tumor positive for one or more EGFR inhibitor resistance mutations. In some embodiments, the tumor with a dysregulation of an EGFR gene, an EGFR kinase, or expression or activity or level of any of the same is determined using a regulatory agency-approved, e.g., FDA-approved, assay or kit.

In some embodiments of any of the methods or uses described herein, the subject has a clinical record indicating that the subject has a tumor that has a dysregulation of an EGFR gene, an EGFR kinase, or expression or activity or level of any of the same (e.g., a tumor having one or more EGFR inhibitor resistance mutations). Also provided are methods of treating a subject that include administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, to a subject having a clinical record that indicates that the subject has a dysregulation of an EGFR gene, an EGFR kinase, or expression or activity or level of any of the same.

In some embodiments, the methods provided herein include performing an assay on a sample obtained from the subject to determine whether the subject has a dysregulation of an EGFR gene, an EGFR protein, or expression or level of any of the same. In some such embodiments, the method also includes administering to a subject determined to have a dysregulation of an EGFR gene, an EGFR protein, or expression or activity, or level of any of the same a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the method includes determining that a subject has a dysregulation of an EGFR gene, an EGFR protein, or expression or level of any of the same via an assay performed on a sample obtained from the subject. In such embodiments, the method also includes administering to a subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the dysregulation in an EGFR gene, an EGFR kinase protein, or expression or activity or level of any of the same is one or more point mutation in the EGFR gene (e.g., any of the one or more of the EGFR point mutations described herein). The one or more point mutations in an EGFR gene can result, e.g., in the translation of an EGFR protein having one or more of the following amino acid substitutions, deletions, and insertions: G719S, G719C, G719A, L747S, D761Y, T790M, T854A, L858R, L861Q, a deletion in exon 19 (e.g., L747_A750del), and an insertion in exon 20 (e.g., V769_D770insX, D770_N771insX, N771_P772insX, P772_H773insX, and H773_V774insX). The one or more mutations in an EGFR gene can result, e.g., in the translation of an EGFR protein having one or more of the following amino acid substitutions or deletions: L858R, deletions in exon 19 (e.g., L747_A750del), L747S, D761Y, T790M, and T854A. In some embodiments, the dysregulation in an EGFR gene, an EGFR kinase protein, or expression or activity or level of any of the same is one or more EGFR inhibitor resistance mutations (e.g., any combination of the one or more EGFR inhibitor resistance mutations described herein). In some embodiments, the dysregulation in an EGFR gene, an EGFR kinase protein, or expression or activity or level of any of the same is one or more EGFR exon 20 insertions (e.g., any of the exon 20 insertions described herein). In some embodiments, the EGFR kinase protein insertion is an exon 20 insertion selected from the group consisting of: V769_D770insX, D770_N771insX, N771_P772insX, P772_H773insX, and H773_V774insX. In some embodiments, the EGFR kinase protein insertion is an exon 20 insertion selected from the group consisting of: V769_D770insX, D770_N771insX, N771_P772insX, P772_H773insX, and H773_V774insX. In some embodiments, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP. Some embodiments of these methods further include administering to the subject another anticancer agent (e.g., a second EGFR inhibitor, a second compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or immunotherapy).

In some embodiments of any of the methods or uses described herein, an assay used to determine whether the subject has a dysregulation of an EGFR gene, or an EGFR kinase, or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of an EGFR gene, an EGFR kinase, or expression or activity or levels of any of the same (see, e.g., the references cited herein). In some embodiments, the dysregulation of the EGFR gene, the EGFR kinase, or expression or activity or level of any of the same includes one or more EGFR inhibitor resistance mutations. In some embodiments, the sample is a biological sample or a biopsy sample (e.g., a paraffin-embedded biopsy sample) from the subject. In some embodiments, the subject is a subject suspected of having an EGFR-associated cancer, a subject having one or more symptoms of an EGFR-associated cancer, and/or a subject that has an increased risk of developing an EGFR-associated cancer).

In some embodiments, dysregulation of an EGFR gene, an EGFR kinase, or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the dysregulation of an EGFR gene, an EGFR kinase, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of an EGFR gene, an EGFR kinase, or the expression or activity or level of any of the same. In some embodiments, liquid biopsies can be used to detect the presence of dysregulation of an EGFR gene, an EGFR kinase, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of an EGFR gene, an EGFR kinase, or the expression or activity or level of any of the same.

The term “HER2-associated disease or disorder” as used herein refers to diseases or disorders associated with or having a dysregulation of a HER2 gene, a HER2 kinase, or the expression or activity or level of any (e.g., one or more) of the same (e.g., any of the types of dysregulation of a HER2 gene, a HER2 kinase, a HER2 kinase domain, or the expression or activity or level of any of the same described herein). Non-limiting examples of a HER2-associated disease or disorder include, for example, cancer.

The term “HER2-associated cancer” as used herein refers to cancers associated with or having a dysregulation of a HER2 gene, a HER2 kinase (also called herein a HER2 protein), or expression or activity, or level of any of the same. Non-limiting examples of a HER2-associated cancer are described herein.

In some embodiments, the EGFR-associated cancer is also a HER2-associated cancer. For example, an EGFR-associated cancer can also have a dysregulation of a HER2 gene, a HER2 kinase, or the expression or activity or level of any of the same.

The phrase “dysregulation of a HER2 gene, a HER2 kinase, or the expression or activity or level of any of the same” refers to a genetic mutation (e.g., a mutation in a HER2 gene that results in the expression of a HER2 protein that includes a deletion of at least one amino acid as compared to a wild type HER2 protein, a mutation in a HER2 gene that results in the expression of a HER2 protein with one or more point mutations as compared to a wild type HER2 protein, a mutation in a HER2 gene that results in the expression of a HER2 protein with at least one inserted amino acid as compared to a wild type HER2 protein, a gene duplication that results in an increased level of HER2 protein in a cell, or a mutation in a regulatory sequence (e.g., a promoter and/or enhancer) that results in an increased level of HER2 protein in a cell), an alternative spliced version of a HER2 mRNA that results in a HER2 protein having a deletion of at least one amino acid in the HER2 protein as compared to the wild-type HER2 protein), or increased expression (e.g., increased levels) of a wild type HER2 kinase in a mammalian cell due to aberrant cell signaling and/or dysregulated autocrine/paracrine signaling (e.g., as compared to a control non-cancerous cell). As another example, a dysregulation of a HER2 gene, a HER2 protein, or expression or activity, or level of any of the same, can be a mutation in a HER2 gene that encodes a HER2 protein that is constitutively active or has increased activity as compared to a protein encoded by a HER2 gene that does not include the mutation. Non-limiting examples of HER2 kinase protein fusions and point mutations/insertions/deletions are described in Tables 3-5. Such mutation and overexpression is associated with the development of a variety of cancers (Moasser. Oncogene. 2007 Oct. 4; 26(45): 6469-6487).

Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for treating diseases and disorders such as HER2-associated diseases and disorders, e.g., proliferative disorders such as cancers, including hematological cancers and solid tumors (e.g., advanced solid tumors).

In some embodiments, dysregulation of a HER2 gene, a HER2 kinase, or the expression or activity or level of any of the same can be caused by an activating mutation in a HER2 gene. The exemplary HER2 kinase fusions or point mutations, insertions, and deletions shown in Tables 3-5 can be caused by an activating mutation.

The term “activating mutation” in reference to HER2 describes a mutation in a HER2 gene that results in the expression of a HER2 kinase that has an increased kinase activity, e.g., as compared to a wild type HER2 kinase, e.g., when assayed under identical conditions. For example, an activating mutation can be a mutation in a HER2 gene (that results in the expression of a HER2 kinase that has one or more (e.g., two, three, four, five, six, seven, eight, nine, or ten) amino acid substitutions (e.g., any combination of any of the amino acid substitutions described herein) that has increased kinase activity, e.g., as compared to a wild type HER2 kinase, e.g., when assayed under identical conditions. In another example, an activating mutation can be a mutation in a HER2 gene that results in the expression of a HER2 kinase that has one or more (e.g., two, three, four, five, six, seven, eight, nine, or ten) amino acids deleted, e.g., as compared to a wild type HER2 kinase, e.g., when assayed under identical conditions. In another example, an activating mutation can be a mutation in a HER2 gene that results in the expression of a HER2 kinase that has at least one (e.g., at least 2, at least 3, at least 4, at least 5, at least 6, at least 7, at least 8, at least 9, at least 10, at least 12, at least 14, at least 16, at least 18, or at least 20) amino acid inserted as compared to a wild type HER2 kinase, e.g., the exemplary wild type HER2 kinase described herein, e.g., when assayed under identical conditions. Additional examples of activating mutations are known in the art.

The term “wild type HER2” or “wild-type HER2 kinase” describes a HER2nucleic acid (e.g., a HER2 gene or a HER2 mRNA) or protein (e.g., a HER2 protein) that is found in a subject that does not have a HER2-associated disease, e.g., a HER2-associated cancer (and optionally also does not have an increased risk of developing a HER2-associated disease and/or is not suspected of having a HER2-associated disease), or is found in a cell or tissue from a subject that does not have a HER2-associated disease, e.g., a HER2-associated cancer (and optionally also does not have an increased risk of developing a HER2-associated disease and/or is not suspected of having a HER2-associated disease).

Provided herein is a method of treating a HER2-associated cancer (in a subject in need of such treatment, the method comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. For example, provided herein are methods for treating a HER2-associated cancer in a subject in need of such treatment, the method comprising a) detecting a dysregulation of a HER2 gene, a HER2 kinase, or the expression or activity or level of any of the same in a sample from the subject; and b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the dysregulation of a HER2 gene, a HER2 kinase, or the expression or activity or level of any of the same includes one or more HER2 kinase protein point mutations/insertions. Non-limiting examples of HER2 kinase protein fusions and point mutations/insertions/deletions are described in Tables 3-5. In some embodiments, the HER2 kinase protein point mutations/insertions/deletions are selected from the group consisting of S310F, S310Y, R678Q, R678W, R678P, 1767M, V773M, V777L, V842I, Y772_A775dup, A775_G776insYVMA, G776delinsVC, G776delinsVV, V777_G778insGSP, and P780_Y781insGSP. In some embodiments, the HER2 kinase protein point mutations/insertions/deletions are exon 20 point mutations/insertions/deletions selected from the group consisting of V773M, G776C, G776V, G776S, V777L, V777M, S779T, P780L, S783P, M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, and P780_Y781insGSP. In some embodiments, the HER2 kinase protein point mutations/insertions/deletions are exon 20 point mutations/insertions/deletions selected from the group consisting of Y772_A775dup, A775_G776insYVMA, G776delinsVC, G776delinsVV, V777_G778insGSP, and P780_Y781insGSP.

In some embodiments of any of the methods or uses described herein, the cancer (e.g., HER2-associated cancer) is selected from a hematological cancer (e.g., Hodgkin lymphoma, non-Hodgkin lymphoma, and leukemia such as acute-myelogenous leukemia (AML), chronic-myelogenous leukemia (CML), acute-promyelocytic leukemia, and acute lymphocytic leukemia (ALL)), alveolar rhabdomyosarcoma, central or peripheral nervous system tissue cancer, an endocrine or neuroendocrine cancer including multiple neuroendocrine type I and type II tumors, Li-Fraumeni tumors, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, tracheal cancer, oral cancer, oropharyngeal cancer, nasopharyngeal cancer, respiratory cancer, urogenital cancer, cancer of the vulva, colon cancer, esophageal cancer, cervical cancer, gastrointestinal carcinoid tumor, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer including pancreatic islet cell cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, parathyroid cancer, pituitary tumors, adrenal gland tumors, ureter cancer, biliary cancer, and urinary bladder cancer. In some embodiments, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In some embodiments, the cancer is pancreatic cancer, head and neck cancer, melanoma, colon cancer, renal cancer, leukemia, lung cancer, or breast cancer. In some cases, the cancer is melanoma, colon cancer, renal cancer, leukemia, or breast cancer.

In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, Liu et al. J Exp Clin Cancer Res. 2019 May 23; 38(1):219); and Ding et al. Cancer Res. 2003 Mar. 1; 63(5):1106-13). In some embodiments, the brain tumor is a primary brain tumor. In some embodiments, the brain tumor is a metastatic brain tumor, e.g., a metastatic brain tumor from lung cancer, melanoma, breast cancer, ovarian cancer, colorectal cancer, kidney cancer, bladder cancer, or undifferentiated carcinoma. In some embodiments, the brain tumor is a metastatic brain tumor from lung cancer (e.g., non-small cell lung cancer). In some embodiments, the compounds provided herein exhibit brain and/or central nervous system (CNS) penetrance. In some embodiments, the patient has previously been treated with another anticancer agent, e.g., another EGFR and/or HER2 inhibitor (e.g., a compound that is not a compound of Formula I) or a multi-kinase inhibitor.

In some embodiments, the cancer is a cancer of B cell origin. In some embodiments, the cancer is a lineage dependent cancer. In some embodiments, the cancer is a lineage dependent cancer where HER2 or the dysregulation of an HER2 gene, an HER2 kinase, or expression or activity or level of any of the same, plays a role in the initiation and/or development of the cancer.

Also provided herein is a method for treating a subject diagnosed with or identified as having a HER2-associated cancer, e.g., any of the exemplary HER2-associated cancers disclosed herein, comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof, as defined herein.

In some embodiments, the dysregulation of a HER2 gene, a HER2 kinase, or expression or activity or level of any of the same, includes one or more deletions (e.g., deletion of an amino acid at position 12), insertions, or point mutation(s) in a HER2 kinase. In some embodiments, the dysregulation of a HER2 gene, a HER2 kinase, or expression or activity or level of any of the same, includes a deletion of one or more residues from the HER2 kinase, resulting in increased signaling activity of HER2.

In some embodiments, the dysregulation of a HER2 gene, a HER2 kinase, or expression or activity or level of any of the same, includes at least one point mutation in a HER2 gene that results in the production of a HER2 kinase that has one or more amino acid substitutions, insertions, or deletions as compared to the wild-type HER2 kinase (see, for example, the point mutations listed in Table 3). In some embodiments, dysregulation of a HER2 gene, a HER2 kinase, or expression or activity or level of any of the same, includes at least one point mutation in a HER2 gene that results in the production of a HER2 kinase that has one or more of the amino acid substitutions, insertions, or deletions in Table 3.

In some embodiments, the dysregulation of an HER2 gene, an HER2 kinase, or expression or activity or level of any of the same, includes an insertion of one or more residues in exon 20 of the HER2 gene (e.g., any of the exon 20 insertions described in Table 1a and Table 1b). Exon 20 of HER2 has two major regions, the c-helix (residues 770-774) and the loop following the c-helix (residues 775-783). In some embodiments, the dysregulation of an HER2 gene, an HER2 kinase, or expression or activity or level of any of the same, includes an insertion of one or more residues in exon 20 selected from the group consisting of: Y772_A775dup, A775_G776insYVMA, G776delinsVC, G776delinsVV, V777_G778insGSP, and P780_Y781insGSP.

TABLE 3
HER2 Protein Amino Acid Substitutions/Insertions/Deletions^{A}

| Amino Acid | Non-Limiting Exemplary | Non-limiting Exemplary HER2- |
|---|---|---|
| Position(s) | Mutations | associated Cancer(s) |
| 122 | P122L¹¹ | Metastatic Colorectal Cancer |
| 263 | I263T⁷ | Colorectal Cancer⁷ |
| 265 | E265K¹¹ | Metastatic Colorectal Cancer |
| 270 | A270S⁶ | Breast Cancer |
| 292 | G292R¹¹ | Metastatic Colorectal Cancer |
| 309 | G309A³, G309E¹⁵ | Breast Cancer |
| 310 | S310F⁷, S310Y⁸ | Colorectal Cancer⁷ |
| 311 | C311R⁸ | |
| 313 | L313V¹¹ | Metastatic Colorectal Cancer |
| 429 | S429R¹⁵, S429H¹⁵ | |
| 466 | A466T⁷ | Colorectal Cancer⁷ |
| 648 | A648V¹⁰ | Urinary Cancer |
| 650 | P650L¹⁰, P650S¹⁰ | Melanoma, Uterine Cancer |
| 651 | L651V¹⁰ | Breast Cancer, Cervical Cancer |
| 652 | T652M¹⁰, T652R¹⁰ | Lung Cancer, Colorectal cancer, |
| | | Liver cancer, Head and Neck |
| | | Cancer, Endometrial Cancer, |
| | | Ovarian Cancer |
| 653 | S653C¹⁰, S653P¹⁰ | Breast Cancer¹⁰, Urinary Cancer, |
| | | Breast Cancer, Colorectal Cancer, |
| | | Liver Cancer, Metastatic |
| | | Colorectal Cancer¹¹ |
| 654 | I654T¹⁰, I654M¹⁰, I654L¹⁰ | Lower Gastrointestinal Cancer, |
| | | Neuroendocrine Cancer, Breast |
| | | Cancer, Esophageal Cancer, Soft |
| | | Tissue Cancer |
| 655 | I655M¹⁰, I655V¹⁴ | Lung Cancer, Colorectal Cancer, |
| | | Ovarian Cancer, Urinary Cancer |
| 656 | S656C¹⁰ | Esophageal Cancer |
| 657 | A657V¹⁰ | Prostate Caner, Colorectal Cancer |
| 659 | V659E¹⁰, V659D¹⁰, V659L¹⁰, | Lung cancer¹⁰, Biliary Cancer, |
| | V659F¹⁰, | Colorectal Cancer, Breast Cancer, |
| | | Metastatic Colorectal Cancer¹¹ |
| 659-661 | V659_I661 > VVEGI¹⁰ | Lung Cancer |
| 659-660 | V659_G660 > ER¹⁰ | Lung Cancer |
| 660 | G660D¹⁰ | Biliary Cancer¹⁰, Lung Cancer, |
| | | Urniary Cancer, Colorectal Cancer, |
| | | Glioma, Lower Gastrointestinal |
| | | Cancer, Gastric Cancer, Liver |
| | | Cancer, Metastatic Colorectal |
| | | Cancer¹¹ |
| 661 | I661V¹⁰ | Colorectal Cancer |
| 662 | L662V¹⁰ | Cervical Cancer, Lung Cancer, |
| | | Breast Cancer |
| 663 | L663P¹⁰ | Soft Tissue Cancer |
| 664 | V664F¹⁰, V664I¹⁰ | Lung Cancer, Breast Cancer, |
| | | Gastric Cancer |
| 665 | V665M¹⁰ | Prostate Cancer, Colorectal Cancer |
| 665-666 | V665_V666del¹⁰ | Breast Cancer |
| 666 | V666I¹⁰ | Colorectal Cancer |
| 667 | L667*¹⁰, L667S¹⁰ | Breast Cancer, Soft Tissue Cancer |
| 668 | G668E¹⁰, G668R¹⁰ | Glioma, Lung Cancer |
| 669 | V669A¹⁰, V669L¹⁰ | Biliary Cancer, Breast Cancer, |
| | | Glioma |
| 672 | G672R¹⁰ | SSC other, Lung Cancer |
| 673 | I673F¹⁰, I673M¹⁰, I673V¹⁰ | Colorectal Cancer, Lung Cancer |
| 674 | L674V¹⁰, L674I¹⁰ | Colorectal Cancer, Lung Cancer |
| 675 | I675M¹⁰, I675T¹⁰, I675L¹⁰ | Lung Cancer, Urinary Cancer, |
| | | Thyroid Cancer, |
| 676 | K676M¹⁰ | Lung Cancer |
| 677 | R677*¹⁰, R677L¹⁰, R677Q¹⁰ | Lung Cancer, Endometrial Cancer, |
| | | Pancreatic Cancer, Neuroendocrine |
| | | Cancer, Colorectal Cancer, |
| | | Glioma, Myeloma |
| 678 | R678Q⁷, R678W¹⁰, R678P¹⁰ | Colorectal Cancer⁷, Gastric |
| | | Cancer¹⁰, Biliary Cancer¹⁰, Urinary |
| | | Cancer¹⁰, Ovarian Cancer¹⁰, |
| | | Endometrial Cancer¹⁰, Carcinoma |
| | | of Unknown Primary¹⁰, Lung |
| | | Cancer¹⁰, Appendicidal Cancer¹⁰, |
| | | Pancreatic Cancer¹⁰, Breast Cancer¹⁰, |
| | | Neuroendocrine Cancer¹⁰, |
| | | Cervical Cancer¹⁰, Lower |
| | | Gastrointestinal Cancer¹⁰, Prostate |
| | | Cancer¹⁰, Liver Cancer¹⁰, Central |
| | | Nervous System Cancer (Non- |
| | | Glioma)¹⁰, Melanoma¹⁰, Salivary |
| | | Gland Cancer¹⁰, Metastatic |
| | | Colorectal Cancer¹¹ |
| 679 | Q679E¹⁰, Q679H¹⁰ | Pancreatic Cancer, Colorectal |
| | | Cancer |
| 680 | Q680del¹⁰ | Cervical Cancer |
| 681 | K681N¹⁰ | Lung Cancer |
| 682 | I682T¹⁰, I682M¹⁰ | Endometrial Cancer, Colorectal |
| | | Cancer |
| 683 | R683W¹⁰, R683Q¹⁰ | Breast Cancer, Pancreatic Cancer, |
| | | Endometrial Cancer, Bone Cancer, |
| | | Colorectal Cancer, |
| 684 | K684N¹⁰ | Colorectal Cancer |
| 685 | Y685H¹⁰ | Colorectal Cancer, Carcinoma of |
| | | Unknown Primary |
| 686 | T686M¹⁰, T686A¹⁰, T686R¹⁰ | Colorectal Cancer, Urinary Cancer, |
| | | Ovarian Cancer, Endometrial |
| | | Cancer |
| 688 | R688L¹⁰, R688W¹⁰, R688Q¹⁰ | Skin Cancer, Colorectal Cancer, |
| | | Melanoma |
| 689 | R689K¹⁰, R689I¹⁰ | Lung Cancer, Endometrial Cancer |
| 691 | L691R¹⁰ | Endometrial Cancer |
| 693 | E693K¹⁰, E693G¹⁰ | Ovarian Cancer, Melanoma, |
| | | Colorectal Cancer, Breast Cancer, |
| | | Carcinoma of Unknown Primary |
| 694 | T694M¹⁰, T694S¹⁰ | Carcinoma of Unknown Primary, |
| | | Colorectal Cancer, Pancreatic |
| | | Cancer |
| 695 | E695K¹⁰ | Melanoma, Urinary Cancer |
| 697 | V697L¹⁰, V697M¹⁰ | Breast Cancer, Lung Cancer, |
| | V697del¹⁰ | Ovarian Cancer, Lower |
| | | Gastrointestinal Cancer, Colorectal |
| | | Cancer, Skin Cancer, Pancreatic |
| | | Cancer, Salivary Gland Cancer, |
| | | Carcinoma of Unknown Primary, |
| | | Cervical Cancer, Endometrial |
| | | Cancer, Gastric Cancer, |
| 699 | P699del¹⁰, P699S | Endometrial Cancer |
| 701 | T701I¹⁰ | Ovarian Cancer |
| 702 | P702S¹⁰, P702L¹⁰ | Gastric Cancer, Lower |
| | | Gastrointestinal Cancer, |
| | | Carcinoma of Unknown Primary, |
| | | Endometrial Cancer, Breast |
| | | Cancer, Ovarian Cancer, Liver |
| | | Cancer |
| 704 | G704E¹⁰, G704R¹⁰ | Glioma, Colorectal Cancer |
| 705 | A705V¹⁰ | Colorectal Cancer, Soft Tissue |
| | | Cancer |
| 706 | M706V¹⁰ | Breast Cancer |
| 707 | P707L¹⁰ | Soft Tissue Cancer |
| 709 | Q709L¹⁰, Q709K¹⁰ | Glioma, Lung Cancer, Lower |
| | | Gastrointestinal Cancer |
| 710 | A710V¹⁰ | Lung Cancer |
| 711 | Q711H¹⁰ | Breast Cancer, Lung Cancer |
| 712 | M712L¹⁰ | Neuroendocrine Cancer, |
| | | Esophageal Cancer |
| 713 | R713Q¹⁰, R713W¹⁰, R713L¹⁰ | Prostate Cancer, Lung Cancer, |
| | | Endometrial Cancer |
| 726 | L726I¹⁵, L726F¹⁵ | |
| 733 | 733I¹¹ | Metastatic Colorectal Cancer¹¹ |
| 755 | L755S^{2,7}, L755W³, L755P⁸ | Lung Adenocarcinoma², Breast |
| | L755F¹⁴ | Cancer³, Bone Metastases From |
| | | Breast Cancer⁵, Colorectal |
| | | Cancer⁷, Metastatic Colorectal |
| | | Cancer¹¹ |
| 755-759 | del. 755-7593 | Breast Cancer³ |
| 760 | S760A¹⁵ | |
| 767 | I767M¹¹ | Metastatic Colorectal Cancer¹¹ |
| 769 | D769H², D769Y³, D769N¹¹ | Lung Adenocarcinoma², Breast |
| | | Cancer³, Metastatic Colorectal |
| | | Cancer¹¹ |
| 773 | V773M¹¹ | Metastatic Colorectal Cancer¹¹ |
| 776 | G776C², G776V⁸, G776S¹¹ | Lung Adenocarcinoma, Metastatic |
| | | Colorectal Cancer¹¹ |
| 777 | V777L^{2,7}, V777M² | Lung Adenocarcinoma², Breast |
| | | Cancer³, Bone Metastases From |
| | | Breast Cancer⁵, Colorectal |
| | | Cancer⁷, Metastatic Colorectal |
| | | Cancer¹¹ |
| 779 | S779T⁸ | Breast Cancer |
| 780 | P780ins3, P780L¹⁵ | Breast Cancer³ |
| 783 | S783P2 | Breast Cancer |
| 784 | R784G¹² | Metastatic Colorectal Cancer¹² |
| 785 | L785F¹⁵ | Breast Cancer |
| 798 | T798I⁴, T798M¹⁵ | Breast Cancer |
| 835 | Y835F¹⁵ | |
| 838 | R838Q¹⁵ | |
| 842 | V842I^{3,7} | Breast Cancer³, Colorectal |
| | | Cancer⁷, Metastatic Colorectal |
| | | Cancer¹¹ |
| 862 | T862A¹¹ | Metastatic Colorectal Cancer¹¹ |
| 866 | 866M⁷ | Colorectal Cancer⁷ |
| 868 | R868W⁷ | Colorectal Cancer⁷ |
| 869 | L869R⁴ | Breast Cancer |
| 869 + 798 | L869R + T798I⁴ | Breast Cancer |
| 878 | H878Y¹¹ | Metastatic Colorectal Cancer¹¹ |
| 887 | M887I¹⁵ | |
| 896 | R896C³, R896H¹¹ | Breast Cancer³, Metastatic |
| | | Colorectal Cancer¹¹ |
| 896 + 755 | R896C + L755W³ | Breast Cancer³ |
| 1136 | Q1136fs*5¹¹ | Metastatic Colorectal Cancer¹¹ |
| 1170 | P1170fs*88+¹¹ | Metastatic Colorectal Cancer¹¹ |
| 1189 | G1189fs*9¹¹ | Metastatic Colorectal Cancer¹¹ |
| 1201 | G1201V¹⁵ | |
| 1219 | N1219S⁷ | Colorectal Cancer⁷ |
| 1232 | A1232fs*25+¹¹ | Metastatic Colorectal Cancer¹¹ |
| Exon 16 | Deletion¹¹ | Metastatic Colorectal Cancer¹¹ |

Exon 20 deletions and insertions

| | |
|---|---|
| M774AYVM¹³ | Non-Small Cell Lung Cancer¹³ |
| M774delinsWLV⁹ | Non-Small Cell Lung Cancer⁹ |
| A775_G776insYVMA | Lung Adenocarcinoma |
| (c.2324_2325ins12)¹ | |
| A775_G776insAVMA | Lung Adenocarcinoma |
| (c.2324_2325ins12)¹⁴ | |
| A775_G776insSVMA⁹ | Non-Small Cell Lung Cancer⁹ |
| A775_G776insVAG¹⁴ | Lung Adenocarcinoma |
| A775insV G776C⁸ | |
| A775_G776insI⁹ | Non-Small Cell Lung Cancer⁹ |
| G776delinsVC^{2,8}, G776del | Lung Adenocarcinoma |
| insVV⁸; G776delinsLC⁹ | |
| G776C_V777insC⁸; | |
| G776CV777insV⁸ | |
| V777_G778insCG², | Lung Adenocarcinoma², |
| V777_G778insGSP¹⁶ | Non-Small Cell Lung Cancer¹⁶ |
| G778_S779insCPG⁹ | Non-Small Cell Lung Cancer⁹ |
| P780_Y781insGSP^{2,8} | Lung Adenocarcinoma |

^{A}The HER2 mutations shown may be activating mutations and/or confer increased resistance of HER2 to a HER2 inhibitor and/or a multi-kinase inhibitor (MKI), e.g., as compared to a wildtype HER2.
¹Li et al. J Thorac Oncol. 2016 March; 11(3):414-9.
²Arcila et al. Clin Cancer Res. 2012 Sep. 15; 18(18): 10.1158/1078-0432.CCR-12-0912.
³Bose et al. Cancer Discov. 2013 February; 3(2):224-37.
⁴Hanker et al. Cancer Discov. 2017 June; 7(6):575-585.
⁵Christgen et al. Virchows Arch. 2018 November; 473(5):577-582.
⁶Si et al. Cancer Biomark. 2018; 23(2):165-171.
⁷Kavuri et al. Cancer Discov. 2015 August; 5(8): 832-841.
⁸Robichaux et al. Nat Med. 2018 May; 24(5): 638-646.
⁹Kosaka et al. Cancer Res. 2017 May 15; 77(10): 2712-2721.
¹⁰Pahuja et al. Cancer Cell. 2018 Nov. 12; 34(5): 792-806.e5.
¹¹Ross et al. Cancer. 2018 Apr. 1;124(7):1358-1373.
¹²Gharib et al. J Cell Physiol. 2019 August; 234(8):13137-13144.
¹³Krawczyk et al. Oncol Lett. 2013 October; 6(4): 1063-1067.
¹⁴Lai et al. Eur J Cancer. 2019 March; 109: 28-35.
¹⁵Sun et al. J Cell Mol Med. 2015 December; 19(12): 2691-2701.
¹⁶Xu et al. Thorac Cancer. 2020 March; 11(3):679-685.

In some embodiments, the dysregulation of a HER2 gene, a HER2 kinase, or expression or activity or level of any of the same, includes a splice variation in a HER2 mRNA which results in an expressed protein that is an alternatively spliced variant of HER2 having at least one residue deleted (as compared to the wild-type HER2 kinase) resulting in a constitutive activity of a HER2 kinase domain. In some embodiments, the splice variant of HER2 is Δ16HER-3 or p95HER-2. See, e.g., Sun et al. J Cell Mol Med. 2015 December; 19(12): 2691-2701.

In some embodiments, dysregulation of an HER2 gene, an HER2 kinase, or the expression or activity or level of any of the same can be caused by a splice variation in a HER2 mRNA that results in the expression of an altered HER2 protein that has increased resistance to inhibition by an HER2 inhibitor, a tyrosine kinase inhibitor (TKI), and/or a multi-kinase inhibitor (MKI), e.g., as compared to a wild type HER2 kinase (e.g., the HER2 variants described herein). See, e.g., Rexer and Arteaga. Crit Rev Oncog. 2012; 17(1): 1-16.

In some embodiments, the dysregulation of a HER2 gene, a HER2 kinase, or expression or activity or level of any of the same, includes one or more chromosome translocations or inversions resulting in HER2 gene fusions, respectively. In some embodiments, the dysregulation of a HER2 gene, a HER2 kinase, or expression or activity or level of any of the same, is a result of genetic translocations in which the expressed protein is a fusion protein containing residues from a non-HER2 partner protein and HER2, and include a minimum of a functional HER2 kinase domain, respectively.

TABLE 4
Exemplary HER2 Fusion Proteins and Cancers

| | Non-limiting |
|---|---|
| | Exemplary |
| Non-limiting Exemplary | HER2-associated |
| HER2 Fusions | Cancer(s) |
| ZNF207_ex2/HER2_ex18¹ | Gastric Cancer |
| MDK_ex4/HER2_ex11¹ | Gastric Cancer |
| NOS2_ex2/HER2_ex2¹ | Gastric Cancer |

¹Yu et al. J Transl Med. 2015; 13: 116.

In some embodiments, the dysregulation of a HER2 gene, a HER2 kinase, or expression or activity or level of any of the same, includes at least one point mutation in a HER2 gene that results in the production of a HER2 kinase that has one or more amino acid substitutions or insertions or deletions in a HER2 gene that results in the production of a HER2 kinase that has one or more amino acids inserted or removed, as compared to the wild-type HER2 kinase.

In other embodiments, the dysregulation of a HER2 gene, a HER2 kinase, or expression or activity or level of any of the same, includes at least one point mutation in a HER2 gene that results in the production of a HER2 kinase that has one or more amino acid substitutions as compared to the wild-type HER2 kinase, and which has increased resistance to a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as compared to a wild type HER2 kinase or a HER2 kinase not including the same mutation.

Exemplary Sequence of Mature Human HER2 Protein (UniProtKB entry P04626) (SEQ ID NO: 2)

MELAALCRWG LLLALLPPGA ASTQVCTGTD
MKLRLPASPE THLDMLRHLY
QGCQVVQGNL ELTYLPTNAS LSFLQDIQEV
QGYVLIAHNQ VRQVPLQRLR
IVRGTQLFED NYALAVLDNG DPLNNTTPVT
GASPGGLREL QLRSLTEILK
GGVLIQRNPQ LCYQDTILWK DIFHKNNQLA
LTLIDTNRSR ACHPCSPMCK
GSRCWGESSE DCQSLTRTVC AGGCARCKGP
LPTDCCHEQC AAGCTGPKHS
DCLACLHFNH SGICELHCPA LVTYNTDTFE
SMPNPEGRYT FGASCVTACP
YNYLSTDVGS CTLVCPLHNQ EVTAEDGTQR
CEKCSKPCAR VCYGLGMEHL
REVRAVTSAN IQEFAGCKKI FGSLAFLPES
FDGDPASNTA PLQPEQLQVF
ETLEEITGYL YISAWPDSLP DLSVFQNLQV
IRGRILHNGA YSLTLQGLGI
SWLGLRSLRE LGSGLALIHH NTHLCFVHTV
PWDQLFRNPH QALLHTANRP
EDECVGEGLA CHQLCARGHC WGPGPTQCVN
CSQFLRGQEC VEECRVLQGL
PREYVNARHC LPCHPECQPQ NGSVTCFGPE
ADQCVACAHY KDPPFCVARC
PSGVKPDLSY MPIWKFPDEE GACQPCPINC
THSCVDLDDK GCPAEQRASP
LTSIISAVVG ILLVVVLGVV FGILIKRRQQ
KIRKYTMRRL LQETELVEPL
TPSGAMPNQA QMRILKETEL RKVKVLGSGA
FGTVYKGIWI PDGENVKIPV
AIKVLRENTS PKANKEILDE AYVMAGVGSP
YVSRLLGICL TSTVQLVTQL
MPYGCLLDHV RENRGRLGSQ DLLNWCMQIA
KGMSYLEDVR LVHRDLAARN
VLVKSPNHVK ITDFGLARLL DIDETEYHAD
GGKVPIKWMA LESILRRRFT
HQSDVWSYGV TVWELMTFGA KPYDGIPARE
IPDLLEKGER LPQPPICTID
VYMIMVKCWM IDSECRPRFR ELVSEFSRMA
RDPQRFVVIQ NEDLGPASPL
DSTFYRSLLE DDDMGDLVDA EEYLVPQQGF
FCPDPAPGAG GMVHHRHRSS
STRSGGGDLT LGLEPSEEEA PRSPLAPSEG
AGSDVFDGDL GMGAAKGLQS
LPTHDPSPLQ RYSEDPTVPL PSETDGYVAP
LTCSPQPEYV NQPDVRPQPP
SPREGPLPAA RPAGATLERP KTLSPGKNGV
VKDVFAFGGA VENPEYLTPQ
GGAAPQPHPP PAFSPAFDNL YYWDQDPPER
GAPPSTFKGT PTAENPEYLG
LDVPV

In some embodiments, dysregulation of an HER2 gene, an HER2 kinase, or expression or activity or level of any of the same, includes at least one HER2 inhibitor resistance mutation in an HER2 gene that results in the production of an HER2 kinase that has one or more of the amino acid substitutions, insertions, or deletions as described in Table 5. In some embodiments, compounds of Formula (I) (e.g., Formula (I-a), (I-b), (I-c), (I-d), (I-e), (I-f), (I-g), (I-h), (I-i), (I-j), or (I-k)) and pharmaceutically acceptable salts and solvates thereof, are useful in treating subjects that develop cancers with HER2 inhibitor resistance mutations (e.g., that result in an increased resistance to a first HER2 inhibitor, e.g., a substitution at amino acid position 755 or 798 (e.g., L755S, L755P, T798I, and T798M), and/or one or more HER2 inhibitor resistance mutations listed in Table 5) by either dosing in combination or as a subsequent or additional (e.g., follow-up) therapy to existing drug treatments (e.g., other inhibitors of HER2; e.g., first and/or second HER2 inhibitors).

TABLE 5
HER2 Protein Amino Acid Resistance Mutations

| | | Non-limiting |
|---|---|---|
| | | Exemplary |
| Amino Acid | Non-Limiting | HER2-associated |
| Position(s) | Exemplary Mutations | Cancer(s) |
| 726 | L726I, L726F² | Breast Cancer |
| 755 | L755S², L755P² | Breast Cancer |
| 780 | P780L² | Breast Cancer |
| 783 | S783P² | Breast Cancer |
| 785 | L785F² | Breast Cancer |
| 798 | T798I¹, T798M² | Breast Cancer |

¹Hanker et al. Cancer Discov. 2017 June; 7(6):575-585.
²Sun et al. J Cell Mol Med. 2015 December; 19(12): 2691-2701.

As used herein, a “first inhibitor of HER2” or “first HER2 inhibitor” is a HER2 inhibitor as defined herein, but which does not include a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as defined herein. As used herein, a “second inhibitor of HER2” or a “second HER2 inhibitor” is a HER2 inhibitor as defined herein, but which does not include a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as defined herein. When both a first and a second inhibitor of HER2 are present in a method provided herein, the first and second inhibitors of HER2 are different. In some embodiments, the first and/or second inhibitor of HER2 bind in a different location than a compound of Formula (I) (e.g., Formula (I-a), (I-b), (I-c), (I-d), (I-e), (I-f), (I-g), (I-h), (I-i), (I-j), or (I-k)). For example, in some embodiments, a first and/or second inhibitor of HER2 can inhibit dimerization of HER2, while a compound of Formula (I) (e.g., Formula (I-a), (I-b), (I-c), (I-d), (I-e), (I-f), (I-g), (I-h), (I-i), (I-j), or (I-k)) can inhibit the active site. In some embodiments, a first and/or second inhibitor of HER2 can be an allosteric inhibitor of HER2, while a compound of Formula (I) (e.g., Formula (I-a), (I-b), (I-c), (I-d), (I-e), (I-f), (I-g), (I-h), (I-i), (I-j), or (I-k)) can inhibit the HER2 active site.

Exemplary first and second inhibitors of HER2 are described herein. In some embodiments, a first or second inhibitor of HER2 can be selected from the group consisting of: trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPRO®), afatinib (GILOTRIF®), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-222611, and AEE-788.

In some embodiments, compounds of Formula (I) (e.g., Formula (I-a), (I-b), (I-c), (I-d), (I-e), (I-f), (I-g), (I-h), (I-i), (I-j), or (I-k)), or pharmaceutically acceptable salts and solvates thereof, are useful for treating a cancer that has been identified as having one or more HER2 inhibitor resistance mutations (that result in an increased resistance to a first or second inhibitor of HER2, e.g., a substitution described in Table 5 including substitutions at amino acid position 755 or 798 (e.g., L755S, L755P, T798I, and T798M)). In some embodiments, the one or more HER2 inhibitor resistance mutations occurs in a nucleic acid sequence encoding a mutant HER2 protein (e.g., a mutant HER2 protein having any of the mutations described in Table 3) resulting in a mutant HER2 protein that exhibits HER2 inhibitor resistance.

Like EGFR, the epidermal growth factor receptor 2 (HER2) belongs to the ErbB family of receptor tyrosine kinases (RTKs) and provides critical functions in epithelial cell physiology (Schlessinger J (2014) Cold Spring Harb Perspect Biol 6, a008912; and Moasser. Oncogene. 2007 Oct. 4; 26(45): 6469-6487). It is frequently mutated and/or overexpressed in different types of human cancers and is the target of multiple cancer therapies currently adopted in the clinical practice (Moasser. Oncogene. 2007 Oct. 4; 26(45): 6469-6487).

Accordingly, provided herein are methods for treating a subject identified or diagnosed as having a HER2-associated cancer that include administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. In some embodiments, the subject that has been identified or diagnosed as having a HER2-associated cancer through the use of a regulatory agency-approved, e.g., FDA-approved test or assay for identifying dysregulation of a HER2 gene, a HER2 kinase, or expression or activity or level of any of the same, in a subject or a biopsy sample from the subject or by performing any of the non-limiting examples of assays described herein. In some embodiments, the test or assay is provided as a kit. In some embodiments, the cancer is a HER2-associated cancer. Also provided are methods for treating cancer in a subject in need thereof, the method comprising: (a) detecting a HER2-associated cancer in the subject; and (b) administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. Some embodiments of these methods further include administering to the subject another anticancer agent (e.g., a second HER2 inhibitor, a second compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or an immunotherapy). In some embodiments, the subject was previously treated with a first HER2 inhibitor or previously treated with another anticancer treatment, e.g., at least partial resection of the tumor or radiation therapy. In some embodiments, the subject is determined to have a HER2-associated cancer through the use of a regulatory agency-approved, e.g., FDA-approved test or assay for identifying dysregulation of a HER2 gene, a HER2 kinase, or expression or activity or level of any of the same, in a subject or a biopsy sample from the subject or by performing any of the non-limiting examples of assays described herein. In some embodiments, the test or assay is provided as a kit. In some embodiments, the cancer is a HER2-associated cancer.

Also provided are methods of treating a subject that include performing an assay on a sample obtained from the subject to determine whether the subject has a dysregulation of a HER2 gene, a HER2 kinase, or expression or activity or level of any of the same, and administering (e.g., specifically or selectively administering) a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof, to the subject determined to have a dysregulation of a HER2 gene, a HER2 kinase, or expression or activity or level of any of the same. Some embodiments of these methods further include administering to the subject another anticancer agent (e.g., a second HER2 inhibitor, a second compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or immunotherapy). In some embodiments of these methods, the subject was previously treated with a first HER2 inhibitor or previously treated with another anticancer treatment, e.g., at least partial resection of a tumor or radiation therapy. In some embodiments, the subject is a subject suspected of having a HER2-associated cancer, a subject presenting with one or more symptoms of a HER2-associated cancer, or a subject having an elevated risk of developing a HER2-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

As used herein, a “first inhibitor of HER2” or “first HER2 inhibitor” is a HER2 inhibitor as defined herein, which does not include a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as defined herein. As used herein, a “second inhibitor of HER2” or a “second HER2 inhibitor” is an inhibitor of HER2 as defined herein, which does not include a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as defined herein. When both a first and a second HER2 inhibitor are present in a method provided herein, the first and second HER2 inhibitors are different.

Also provided is a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof, for use in treating a HER2-associated cancer in a subject identified or diagnosed as having a HER2-associated cancer through a step of performing an assay (e.g., an in vitro assay) on a sample obtained from the subject to determine whether the subject has a dysregulation of a HER2 gene, a HER2 kinase, or expression or activity or level of any of the same, where the presence of a dysregulation of a HER2 gene, a HER2 kinase, or expression or activity or level of any of the same, identifies that the subject has a HER2-associated cancer. Also provided is the use of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for the manufacture of a medicament for treating a HER2-associated cancer in a subject identified or diagnosed as having a HER2-associated cancer through a step of performing an assay on a sample obtained from the subject to determine whether the subject has a dysregulation of a HER2 gene, a HER2 kinase, or expression or activity or level of any of the same where the presence of dysregulation of a HER2 gene, a HER2 kinase, or expression or activity or level of any of the same, identifies that the subject has a HER2-associated cancer. Some embodiments of any of the methods or uses described herein further include recording in the subject's clinical record (e.g., a computer readable medium) that the subject is determined to have a dysregulation of a HER2 gene, a HER2 kinase, or expression or activity or level of any of the same, through the performance of the assay, should be administered a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for use in the treatment of a cancer in a subject in need thereof, or a subject identified or diagnosed as having a HER2-associated cancer. Also provided is the use of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for the manufacture of a medicament for treating a cancer in a subject identified or diagnosed as having a HER2-associated cancer (. In some embodiments, a subject is identified or diagnosed as having a HER2-associated cancer through the use of a regulatory agency-approved, e.g., FDA-approved, kit for identifying dysregulation of a HER2 gene, a HER2 kinase, or expression or activity or level of any of the same, in a subject or a biopsy sample from the subject. As provided herein, a HER2-associated cancer includes those described herein and known in the art.

In some embodiments of any of the methods or uses described herein, the subject has been identified or diagnosed as having a cancer with a dysregulation of a HER2 gene, a HER2 kinase, or expression or activity or level of any of the same. In some embodiments of any of the methods or uses described herein, the subject has a tumor that is positive for a dysregulation of a HER2 gene, a HER2 kinase, or expression or activity or level of any of the same. In some embodiments of any of the methods or uses described herein, the subject can be a subject with a tumor(s) that is positive for a dysregulation of a HER2 gene, a HER2 kinase, or expression or activity or level of any of the same. In some embodiments of any of the methods or uses described herein, the subject can be a subject whose tumors have a dysregulation of a HER2 gene, a HER2 kinase, or expression or activity or level of any of the same. In some embodiments of any of the methods or uses described herein, the subject is suspected of having a HER2-associated cancer. In some embodiments, provided herein are methods for treating a HER2-associated cancer in a subject in need of such treatment, the method comprising a) detecting a dysregulation of a HER2 gene, a HER2 kinase, or the expression or activity or level of any of the same in a sample from the subject; and b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the dysregulation of a HER2 gene, a HER2 kinase, or the expression or activity or level of any of the same includes one or more HER2 kinase protein point mutations/insertions/deletions. Non-limiting examples of HER2 kinase protein fusions and point mutations/insertions/deletions are described in Tables 3-5. In some embodiments, the HER2 kinase protein point mutations/insertions/deletions are selected from the group consisting of a point mutation at amino acid position 310, 678, 755, 767, 773, 777, or 842 (e.g., S310F, S310Y, R678Q, R678W, R678P, 1767M, V773M, V777L, and V842I) and/or an insertion or deletion at amino acid positions 772, 775, 776, 777, and 780 (e.g., Y772_A775dup, A775_G776insYVMA, G776delinsVC, G776delinsVV, V777_G778insGSP, and P780_Y781insGSP). In some embodiments, the HER2 kinase protein point mutation/insertion/deletion is an exon 20 point mutation/insertion/deletion. In some embodiments, the HER2 exon 20 point mutation/insertion/deletion is a point mutation at amino acid position 773, 776, 777, 779, 780, and 783 (e.g., V773M, G776C, G776V, G776S, V777L, V777M, S779T, P780L, and S783P) and/or an exon 20 insertion/deletion such as an insertion/deletion at amino acid positions 774, 775, 776, 777, 778, and 780. In some embodiments, the HER2 kinase protein insertion is an exon 20 insertion selected from the group consisting of: A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, and P780_Y781insGSP. In some embodiments, the HER2 kinase protein mutation/insertion/deletion is an exon 20 insertion/deletion selected from the group consisting of: is Y772_A775dup, A775_G776insYVMA, G776delinsVC, G776delinsVV, V777_G778insGSP, or P780_Y781insGSP. In some embodiments, the cancer with a dysregulation of a HER2 gene, a HER2 kinase, or expression or activity or level of any of the same is determined using a regulatory agency-approved, e.g., FDA-approved, assay or kit. In some embodiments, the tumor that is positive for a dysregulation of a HER2 gene, a HER2 kinase, or expression or activity or level of any of the same is a tumor positive for one or more HER2 inhibitor resistance mutations. In some embodiments, the tumor with a dysregulation of a HER2 gene, a HER2 kinase, or expression or activity or level of any of the same is determined using a regulatory agency-approved, e.g., FDA-approved, assay or kit.

In some embodiments of any of the methods or uses described herein, the subject has a clinical record indicating that the subject has a tumor that has a dysregulation of a HER2 gene, a HER2 kinase, or expression or activity or level of any of the same. Also provided are methods of treating a subject that include administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, to a subject having a clinical record that indicates that the subject has a dysregulation of a HER2 gene, a HER2 kinase, or expression or activity or level of any of the same.

In some embodiments, the methods provided herein include performing an assay on a sample obtained from the subject to determine whether the subject has a dysregulation of a HER2 gene, a HER2 kinase, or expression or level of any of the same. In some such embodiments, the method also includes administering to a subject determined to have a dysregulation of a HER2 gene, a HER2 kinase, or expression or activity, or level of any of the same a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the method includes determining that a subject has a dysregulation of a HER2 gene, a HER2 kinase, or expression or level of any of the same via an assay performed on a sample obtained from the subject. In such embodiments, the method also includes administering to a subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the dysregulation in a HER2 gene, a HER2 kinase, or expression or activity or level of any of the same is one or more point mutation in the HER2 gene (e.g., any of the one or more of the HER2 point mutations described herein). The one or more point mutations in a HER2 gene can result, e.g., in the translation of a HER2 protein having one or more of the following amino acid substitutions: S310F, S310Y, R678Q, R678W, R678P, I767M, V773M, V777L, and V842I. The one or more point mutations in a HER2 gene can result, e.g., in the translation of a HER2 protein having one or more of the following exon 20 amino acid substitutions: V773M, G776C, G776V, G776S, V777L, V777M, S779T, P780L, and S783P. In some embodiments, the dysregulation in a HER2 gene, a HER2 kinase, or expression or activity or level of any of the same is one or more insertions in the HER2 gene (e.g., any of the one or more of the HER2 insertions described herein). The one or more insertions in a HER2 gene can result, e.g., in the translation of a HER2 protein having one or more of the following exon 20 insertions: M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, and P780_Y781insGSP. In some embodiments, the one or more insertions in a HER2 gene can result, e.g., in the translation of a HER2 protein having one or more of the following exon 20 insertions: Y772_A775dup, A775_G776insYVMA, G776delinsVC, G776delinsVV, V777_G778insGSP, and P780_Y781insGSP. Some embodiments of these methods further include administering to the subject another anticancer agent (e.g., a second HER2 inhibitor, a second compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or immunotherapy).

In some embodiments of any of the methods or uses described herein, an assay used to determine whether the subject has a dysregulation of a HER2 gene, a HER2 kinase, or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a HER2 gene, a HER2 kinase, or expression or activity or levels of any of the same (see, e.g., the references cited herein). In some embodiments, the sample is a biological sample or a biopsy sample (e.g., a paraffin-embedded biopsy sample) from the subject. In some embodiments, the subject is a subject suspected of having a HER2-associated cancer, a subject having one or more symptoms of a HER2-associated cancer, and/or a subject that has an increased risk of developing a HER2-associated cancer.

In some embodiments, dysregulation of a HER2 gene, a HER2 kinase, or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the dysregulation of a HER2 gene, a HER2 kinasev, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a HER2 gene, a HER2 kinase, or the expression or activity or level of any of the same. In some embodiments, liquid biopsies can be used to detect the presence of dysregulation of a HER2 gene, a HER2 kinase, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a HER2 gene, a HER2 kinase, or the expression or activity or level of any of the same.

Also provided is a method for inhibiting EGFR activity in a cell, comprising contacting the cell with a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. Also provided is a method for inhibiting HER2 activity in a cell, comprising contacting the cell with a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. Further provided herein is a method for inhibiting EGFR and HER2 activity in a cell, comprising contacting the cell with a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, to a subject having a cell having aberrant EGFR activity and/or HER2 activity. In some embodiments, the cell is a cancer cell. In some embodiments, the cancer cell is any cancer as described herein. In some embodiments, the cancer cell is an EGFR-associated cancer cell. In some embodiments, the cancer cell is a HER2-associated cancer cell. As used herein, the term “contacting” refers to the bringing together of indicated moieties in an in vitro system or an in vivo system. For example, “contacting” an EGFR kinase with a compound provided herein includes the administration of a compound provided herein to an individual or subject, such as a human, having an EGFR kinase, as well as, for example, introducing a compound provided herein into a sample containing a cellular or purified preparation containing the EGFR kinase.

Also provided herein is a method of inhibiting cell proliferation, in vitro or in vivo, the method comprising contacting a cell with an effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof as defined herein.

Further provided herein is a method of increase cell death, in vitro or in vivo, the method comprising contacting a cell with an effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof as defined herein. Also provided herein is a method of increasing tumor cell death in a subject. The method comprises administering to the subject an effective compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in an amount effective to increase tumor cell death.

The phrase “therapeutically effective amount” means an amount of compound that, when administered to a subject in need of such treatment, is sufficient to (i) treat an EGFR kinase-associated disease or disorder or a HER2 kinase-associated disease or disorder, (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

When employed as pharmaceuticals, the compounds of Formula (I) (e.g., Formula (I-a), (I-b), (I-c), (I-d), (I-e), (I-f), (I-g), (I-h), (I-i), (I-j), or (I-k)), including pharmaceutically acceptable salts or solvates thereof, can be administered in the form of pharmaceutical compositions as described herein.

Also provided herein is a method of treating a subject having a cancer, wherein the method comprises:
(a) determining that a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a therapeutically effective amount of a compound of Formula (I) (e.g., Formula (I-a), (I-b), (I-c), (I-d), (I-e), (1-), (I-g), (I-h), (I-i), (I-j), or (I-k)), or a pharmaceutically acceptable salt thereof, as a monotherapy or in conjunction with another anticancer agent to the subject.

Further provided herein is a method of treating a subject having a cancer, wherein the method comprises:
(a) determining that a cancer cell in a sample obtained from a subject having a cancer and previously administered one or more doses of a first EGFR inhibitor does not have one or more EGFR inhibitor resistance mutations that confer increased resistance to a cancer cell or tumor to treatment with the first EGFR inhibitor that was previously administered to the subject; and
(b) administering additional doses of the first EGFR inhibitor to the subject.

### Combinations

In the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other kinase inhibitors, signal transduction inhibitors and/or monoclonal antibodies. For example, a surgery may be open surgery or minimally invasive surgery. Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can be used prior to administration of an additional therapeutic agent or additional therapy. For example, a subject in need thereof can be administered one or more doses of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for a period of time and then undergo at least partial resection of the tumor. In some embodiments, the treatment with one or more doses of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, reduces the size of the tumor (e.g., the tumor burden) prior to the at least partial resection of the tumor. In some embodiments, a subject in need thereof can be administered one or more doses of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for a period of time and under one or more rounds of radiation therapy. In some embodiments, the treatment with one or more doses of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent, such as a first EGFR inhibitor, a first HER2 inhibitor, or a multi-kinase inhibitor, immunotherapy, or radiation (e.g., radioactive iodine)). In some embodiments, a subject has a cancer (e.g., a locally advanced or metastatic tumor) that is refractory or intolerant to prior therapy (e.g., administration of a chemotherapeutic agent, such as a first EGFR inhibitor, a first HER2 inhibitor, or a multi-kinase inhibitor, immunotherapy, or radiation (e.g., radioactive iodine)). In some embodiments, a subject has a cancer (e.g., a locally advanced or metastatic tumor) that has no standard therapy. In some embodiments, a subject is EGFR inhibitor naïve. For example, the subject is naïve to treatment with a selective EGFR inhibitor. In some embodiments, a subject is not EGFR inhibitor naïve. In some embodiments, a subject is HER2 inhibitor naïve. For example, the subject is naïve to treatment with a selective HER2 inhibitor. In some embodiments, a subject is not HER2 inhibitor naïve. In some embodiments, a subject has undergone prior therapy. For example, treatment with a multi-kinase inhibitor (MKI), an EGFR tyrosine kinase inhibitor (TKI), osimertinib, gefitinib, erlotinib, afatinib, lapatinib, neratinib, AZD-9291, CL-387785, CO-1686, or WZ4002.

In some embodiments of any the methods described herein, the compound of Formula (I) (e.g., Formula (I-a), (I-b), (I-c), (I-d), (I-e), (I-f), (I-g), (I-h), (I-i), (I-j), or (I-k)) (or a pharmaceutically acceptable salt thereof) is administered in combination with a therapeutically effective amount of at least one additional therapeutic agent selected from one or more additional therapies or therapeutic (e.g., chemotherapeutic) agents.

Non-limiting examples of additional therapeutic agents include: other EGFR-targeted therapeutic agents (i.e., a first or second EGFR inhibitor), other HER2-targeted therapeutic agents (i.e., a first or second HER2 inhibitor), RAS pathway targeted therapeutic agents, PARP inhibitors, other kinase inhibitors (e.g., receptor tyrosine kinase-targeted therapeutic agents (e.g., Trk inhibitors or multi-kinase inhibitors)), farnesyl transferase inhibitors, signal transduction pathway inhibitors, checkpoint inhibitors, modulators of the apoptosis pathway (e.g., obataclax); cytotoxic chemotherapeutics, angiogenesis-targeted therapies, immune-targeted agents, including immunotherapy, and radiotherapy.

In some embodiments, the other EGFR-targeted therapeutic is a multi-kinase inhibitor exhibiting EGFR inhibition activity. In some embodiments, the other EGFR-targeted therapeutic inhibitor is selective for an EGFR kinase.

Non-limiting examples of EGFR-targeted therapeutic agents (e.g., a first EGFR inhibitor or a second EGFR inhibitor) include an EGFR-selective inhibitor, a panHER inhibitor, and an anti-EGFR antibody. In some embodiments, the EGFR inhibitor is a covalent inhibitor. In some embodiments, the EGFR-targeted therapeutic agent is osimertinib (AZD9291, merelectinib, TAGRISSO™), erlotinib (TARCEVA®), gefitinib (IRESSA®), cetuximab (ERBITUX®), necitumumab (PORTRAZZA™, IMC-11F8), neratinib (HKI-272, NERLYNX®), lapatinib (TYKERB®), panitumumab (ABX-EGF, VECTIBIX®), vandetanib (CAPRELSA®), rociletinib (CO-1686), olmutinib (OLITA™, HM61713, BI-1482694), naquotinib (ASP8273), nazartinib (EGF816, NVS-816), PF-06747775, icotinib (BPI-2009H), afatinib (BIBW 2992, GILOTRIF®), dacomitinib (PF-00299804, PF-804, PF-299, PF-299804), avitinib (AC0010), AC0010MA EAI045, matuzumab (EMD-7200), nimotuzumab (h-R3, BIOMAb EGFR®), zalutumab, MDX447, depatuxizumab (humanized mAb 806, ABT-806), depatuxizumab mafodotin (ABT-414), ABT-806, mAb 806, canertinib (CI-1033), shikonin, shikonin derivatives (e.g., deoxyshikonin, isobutyrylshikonin, acetylshikonin, β,β-dimethylacrylshikonin and acetylalkannin), poziotinib (NOV120101, HM781-36B), AV-412, ibrutinib, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, avitinib, GW572016, pyrotinib (SHR1258), SCT200, CPGJ602, Sym004, MAb-425, Modotuximab (TAB-H49), futuximab (992 DS), zalutumumab, KL-140, R05083945, IMGN289, JNJ-61186372, LY3164530, Sym013, AMG 595, BDTX-189, avatinib, Disruptin, CL-387785, EGFRBi-Armed Autologous T Cells, and EGFR CAR-T Therapy. In some embodiments, the EGFR-targeted therapeutic agent is selected from osimertinib, gefitinib, erlotinib, afatinib, lapatinib, neratinib, AZD-9291, CL-387785, CO-1686, or WZ4002.

Additional EGFR-targeted therapeutic agents (e.g., a first EGFR inhibitor or a second EGFR inhibitor) include those disclosed in WO 2019/246541; WO 2019/165385; WO 2014/176475; and U.S. Pat. No. 9,029,502, each of which is incorporated by reference in its entirety.

In some embodiments, the other HER2-targeted therapeutic is a multi-kinase inhibitor exhibiting HER2 inhibition activity. In some embodiments, the other HER2-targeted therapeutic inhibitor is selective for a HER2 kinase.

Non-limiting examples of HER2-targeted therapeutic agents (e.g., a first HER2 inhibitor or a second HER2 inhibitor) include a HER2-selective inhibitor, a panHER inhibitor, and an anti-HER2 antibody. Exemplary HER2-targeted therapeutic agents include trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPRO®), afatinib (GILOTRIF®), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-222611, and AEE-788.

Additional HER2-targeted therapeutic agents (e.g., a first HER2 inhibitor or a second HER2 inhibitor) include those disclosed in WO 2019/246541; WO 2019/165385; WO 2014/176475; and U.S. Pat. No. 9,029,502, each of which is incorporated by reference in its entirety.

A “RAS pathway targeted therapeutic agent” as used herein includes any compound exhibiting inactivation activity of any protein in a RAS pathway (e.g., kinase inhibition, allosteric inhibition, inhibition of dimerization, and induction of degradation). Non-limiting examples of a protein in a RAS pathway include any one of the proteins in the RAS-RAF-MAPK pathway or PI3K/AKT pathway such as RAS (e.g., KRAS, HRAS, and NRAS), RAF, BRAF, MEK, ERK, PI3K, AKT, and mTOR. In some embodiments, a RAS pathway modulator can be selective for a protein in a RAS pathway, e.g., the RAS pathway modulator can be selective for RAS (also referred to as a RAS modulator). In some embodiments, a RAS modulator is a covalent inhibitor. In some embodiments, a RAS pathway targeted therapeutic agent is a “KRAS pathway modulator.” A KRAS pathway modulator includes any compound exhibiting inactivation activity of any protein in a KRAS pathway (e.g., kinase inhibition, allosteric inhibition, inhibition of dimerization, and induction of degradation). Non-limiting examples of a protein in a KRAS pathway include any one of the proteins in the KRAS-RAF-MAPK pathway or PI3K/AKT pathway such as KRAS, RAF, BRAF, MEK, ERK, PI3K, AKT, and mTOR. In some embodiments, a KRAS pathway modulator can be selective for a protein in a RAS pathway, e.g., the KRAS pathway modulator can be selective for KRAS (also referred to as a KRAS modulator). In some embodiments, a KRAS modulator is a covalent inhibitor. Non-limiting examples of a KRAS-targeted therapeutic agents (e.g., KRAS inhibitors) include BI 1701963, AMG 510, ARS-3248, ARS1620, AZD4785, SML-8-73-1, SML-10-70-1, VSA9, AA12, and MRTX-849.

Further non-limiting examples of RAS-targeted therapeutic agents include BRAF inhibitors, MEK inhibitors, ERK inhibitors, PI3K inhibitors, AKT inhibitors, and mTOR inhibitors. In some embodiments, the BRAF inhibitor is vemurafenib (ZELBORAF®), dabrafenib (TAFINLAR®), and encorafenib (BRAFTOVI™), BMS-908662 (XL281), sorafenib, LGX818, PLX3603, RAF265, RO5185426, GSK2118436, ARQ 736, GDC-0879, PLX-4720, AZ304, PLX-8394, HM95573, RO5126766, LXH254, or a combination thereof.

In some embodiments, the MEK inhibitor is trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, RO5126766, CS3006, WX-554, PD98059, CI1040 (PD184352), hypothemycin, or a combination thereof.

In some embodiments, the ERK inhibitor is FRI-20 (ON-01060), VTX-11e, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-Oxozeaenol, 5-iodotubercidin, GDC0994, ONC201, or a combination thereof.

In some embodiments, PI3K inhibitor is selected from buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF₁₁₂₆, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, GSK2636771, or a combination thereof.

In some embodiments, the AKT inhibitor is selected from miltefosine (IMPADIVO®), wortmannin, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, miltefosine, perifosine, erucylphophocholine, erufosine, SR13668, OSU-A9, PH-316, PHT-427, PIT-1, DM-PIT-1, triciribine (Triciribine Phosphate Monohydrate), API-1, N-(4-(5-(3-acetamidophenyl)-2-(2-aminopyridin-3-yl)-3H-imidazo[4,5-b] pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, ONC201, or a combination thereof.

In some embodiments, the mTOR inhibitor is selected from MLN0128, AZD-2014, CC-223, AZD2014, CC-115, everolimus (RAD001), temsirolimus (CC₁₋₇₇₉), ridaforolimus (AP-23573), sirolimus (rapamycin), or a combination thereof.

Non-limiting examples of farnesyl transferase inhibitors include lonafarnib, tipifarnib, BMS-214662, L778123, L744832, and FTI-277.

In some embodiments, a chemotherapeutic agent includes an anthracycline, cyclophosphamide, a taxane, a platinum-based agent, mitomycin, gemcitabine, eribulin (HALAVEN™), or combinations thereof.

Non-limiting examples of a taxane include paclitaxel, docetaxel, abraxane, and taxotere.

In some embodiments, the anthracycline is selected from daunorubicin, doxorubicin, epirubicin, idarubicin, and combinations thereof.

In some embodiments, the platinum-based agent is selected from carboplatin, cisplatin, oxaliplatin, nedplatin, triplatin tetranitrate, phenanthriplatin, picoplatin, satraplatin and combinations thereof Non-limiting examples of PARP inhibitors include olaparib (LYNPARZA®), talazoparib, rucaparib, niraparib, veliparib, BGB-290 (pamiparib), CEP 9722, E7016, iniparib, IMP4297, NOV1401, 2X-121, ABT-767, RBN-2397, BMN 673, KU-0059436 (AZD2281), BSI-201, PF-01367338, INO-1001, and JPI-289.

Non-limiting examples of immunotherapy include immune checkpoint therapies, atezolizumab (TECENTRIQ®), albumin-bound paclitaxel. Non-limiting examples of immune checkpoint therapies include inhibitors that target CTLA-4, PD-1, PD-L1, BTLA, LAG-3, A2AR, TIM-3, B7-H3, VISTA, IDO, and combinations thereof. In some embodiments the CTLA-4 inhibitor is ipilimumab (YERVOY®). In some embodiments, the PD-1 inhibitor is selected from pembrolizumab (KEYTRUDA®), nivolumab (OPDIVO®), cemiplimab (LIBTAYO®), or combinations thereof. In some embodiments, the PD-L1 inhibitor is selected from atezolizumab (TECENTRIQ®), avelumab (BAVENCIO®), durvalumab (IMFINZI®), or combinations thereof. In some embodiments, the LAG-3 inhibitor is IMP701 (LAG525). In some embodiments, the A2AR inhibitor is CPI-444. In some embodiments, the TIM-3 inhibitor is MBG453. In some embodiments, the B7-H3 inhibitor is enoblituzumab. In some embodiments, the VISTA inhibitor is JNJ-61610588. In some embodiments, the IDO inhibitor is indoximod. See, for example, Marin-Acevedo, et al., J Hematol Oncol. 11: 39 (2018).

In some embodiments, the additional therapy or therapeutic agent is a combination of atezolizumab and nab-paclitaxel.

Accordingly, also provided herein is a method of treating cancer, comprising administering to a subject in need thereof a pharmaceutical combination for treating cancer which comprises (a) a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, (b) an additional therapeutic agent, and (c) optionally at least one pharmaceutically acceptable carrier for simultaneous, separate or sequential use for the treatment of cancer, wherein the amounts of the compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, and the additional therapeutic agent are together effective in treating the cancer.

In some embodiments, the additional therapeutic agent(s) includes any one of the above listed therapies or therapeutic agents which are standards of care in cancers wherein the cancer has a dysregulation of an EGFR gene, an EGFR protein, or expression or activity, or level of any of the same.

In some embodiments, the additional therapeutic agent(s) includes any one of the above listed therapies or therapeutic agents which are standards of care in cancers wherein the cancer has a dysregulation of a HER2 gene, a HER2 kinase, or expression or activity, or level of any of the same.

These additional therapeutic agents may be administered with one or more doses of the compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art.

Also provided herein is (i) a pharmaceutical combination for treating a cancer in a subject in need thereof, which comprises (a) a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, (b) at least one additional therapeutic agent (e.g., any of the exemplary additional therapeutic agents described herein or known in the art), and (c) optionally at least one pharmaceutically acceptable carrier for simultaneous, separate or sequential use for the treatment of cancer, wherein the amounts of the compound of Formula (I) (e.g., Formula (I-a), (I-b), (I-c), (I-d), (I-e), (I-f), (I-g), (I-h), (I-i), (I-j), or (I-k)), or pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the cancer is an EGFR-associated cancer. For example, an EGFR-associated cancer having one or more EGFR inhibitor resistance mutations. In some embodiments, the cancer is a HER2-associated cancer. For example, a HER2-associated cancer having one or more HER2 inhibitor resistance mutations.

The term “pharmaceutical combination”, as used herein, refers to a pharmaceutical therapy resulting from the mixing or combining of more than one active ingredient and includes both fixed and non-fixed combinations of the active ingredients. The term “fixed combination” means that a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, and at least one additional therapeutic agent (e.g., a chemotherapeutic agent), are both administered to a subject simultaneously in the form of a single composition or dosage. The term “non-fixed combination” means that a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, and at least one additional therapeutic agent (e.g., chemotherapeutic agent) are formulated as separate compositions or dosages such that they may be administered to a subject in need thereof simultaneously, concurrently or sequentially with variable intervening time limits, wherein such administration provides effective levels of the two or more compounds in the body of the subject. These also apply to cocktail therapies, e.g., the administration of three or more active ingredients

Accordingly, also provided herein is a method of treating a cancer, comprising administering to a subject in need thereof a pharmaceutical combination for treating cancer which comprises (a) a compound of Formula (I) (e.g., Formula (I-a), (I-b), (I-c), (I-d), (I-e), (I-f), (I-g), (I-h), (I-i), (I-j), or (I-k)), or pharmaceutically acceptable salt thereof, and (b) an additional therapeutic agent, wherein the compound of Formula (I) (e.g., Formula (I-a), (I-b), (I-c), (I-d), (I-e), (I-f), (I-g), (I-h), (I-i), (I-j), or (I-k)) and the additional therapeutic agent are administered simultaneously, separately or sequentially, wherein the amounts of the compound of Formula (I) (e.g., Formula (I-a), (I-b), (I-c), (I-d), (I-e), (I-f), (I-g), (I-h), (I-i), (I-j), or (I-k)), or pharmaceutically acceptable salt thereof, and the additional therapeutic agent are together effective in treating the cancer. In some embodiments, the compound of Formula (I) (e.g., Formula (I-a), (I-b), (I-c), (I-d), (I-e), (I-f), (I-g), (I-h), (I-i), (I-j), or (I-k)), or pharmaceutically acceptable salt thereof, and the additional therapeutic agent are administered simultaneously as separate dosages. In some embodiments, the compound of Formula (I) (e.g., Formula (I-a), (I-b), (I-c), (I-d), (I-e), (I-f), (I-g), (I-h), (I-i), (I-j), or (I-k)), or pharmaceutically acceptable salt thereof, and the additional therapeutic agent are administered as separate dosages sequentially in any order, in jointly therapeutically effective amounts, e.g., in daily or intermittently dosages. In some embodiments, the compound of Formula (I) (e.g., Formula (I-a), (I-b), (I-c), (I-d), (I-e), (I-f), (I-g), (I-h), (I-i), (I-j), or (I-k)), or pharmaceutically acceptable salt thereof, and the additional therapeutic agent are administered simultaneously as a combined dosage. In some embodiments, the cancer is an EGFR-associated cancer. For example, an EGFR-associated cancer having one or more EGFR inhibitor resistance mutations. In some embodiments, the cancer is a HER2-associated cancer. For example, a HER2-associated cancer having one or more HER2 inhibitor resistance mutations.

In some embodiments, the presence of one or more EGFR inhibitor resistance mutations in a tumor causes the tumor to be more resistant to treatment with a first EGFR inhibitor. Methods useful when an EGFR inhibitor resistance mutation causes the tumor to be more resistant to treatment with a first EGFR inhibitor are described below. For example, provided herein are methods of treating a subject having a cancer that include: identifying a subject having a cancer cell that has one or more EGFR inhibitor resistance mutations; and administering to the identified subject a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, is administered in combination with the first EGFR inhibitor. Also provided are methods of treating a subject identified as having a cancer cell that has one or more EGFR inhibitor resistance mutations that include administering to the subject a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, is administered in combination with the first EGFR inhibitor. In some embodiments, the one or more EGFR inhibitor resistance mutations confer increased resistance to a cancer cell or tumor to treatment with the first EGFR inhibitor. In some embodiments, the one or more EGFR inhibitor resistance mutations include one or more EGFR inhibitor resistance mutations listed in Table 2a and Table 2b. For example, the one or more EGFR inhibitor resistance mutations can include a substitution at amino acid position 718, 747, 761, 790, 797, or 854 (e.g., L718Q, L747S, D761Y, T790M, C797S, and T854A).

For example, provided herein are methods for treating an EGFR-associated cancer in a subject in need of such treatment, the method comprising (a) detecting a dysregulation of an EGFR gene, an EGFR kinase, or the expression or activity or level of any of the same in a sample from the subject; and (b) administering to the subject a therapeutically effective amount of a first EGFR inhibitor, wherein the first EGFR inhibitor is selected from the group consisting of osimertinib, gefitinib, erlotinib, afatinib, lapatinib, neratinib, AZD-9291, CL-387785, CO-1686, or WZ4002. In some embodiments, the methods further comprise (after (b)) (c) determining whether a cancer cell in a sample obtained from the subject has at least one EGFR inhibitor resistance mutation; and (d) administering a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as a monotherapy or in conjunction with another anticancer agent to the subject if the subject has been determined to have a cancer cell that has at least one EGFR inhibitor resistance mutation; or (e) administering additional doses of the first EGFR inhibitor of step (b) to the subject if the subject has not been determined to have a cancer cell that has at least one EGFR inhibitor resistance mutation.

Methods useful when a HER2 activating mutation is present in a tumor are described herein. For example, provided herein are methods of treating a subject having a cancer that include: identifying a subject having a cancer cell that has one or more HER2 activating mutations; and administering to the identified subject a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. Also provided are methods of treating a subject identified as having a cancer that has one or more HER2 activating mutations that include administering to the subject a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the one or more HER2 activating mutations include one or more HER2 activating mutations listed in Tables 3-5.

Methods useful when an activating mutation (e.g., HER2 activating mutation) is present in a tumor in a subject are described herein. For example, provided herein are methods of treating a subject having a cancer that include: identifying a subject having a cancer cell that has one or more HER2 activating mutations; and administering to the identified subject a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof.

Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following Scheme 1, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### Compound Preparation

### Example 1: (S)-2-(3-((6,7-dihydro-5H-pyrrolo[1,2-b][1,2,4]triazol-5-yl)methoxy)pyridin-4-yl)-3-((3-fluoro-2-methoxyphenyl)amino)-1,5,6,7-tetrahydro-4H-pyrrolo[3,2-c]pyridin-4-one (Compound 133a)

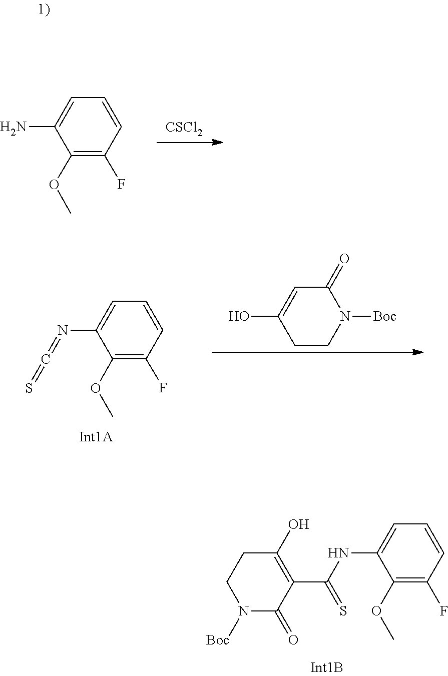

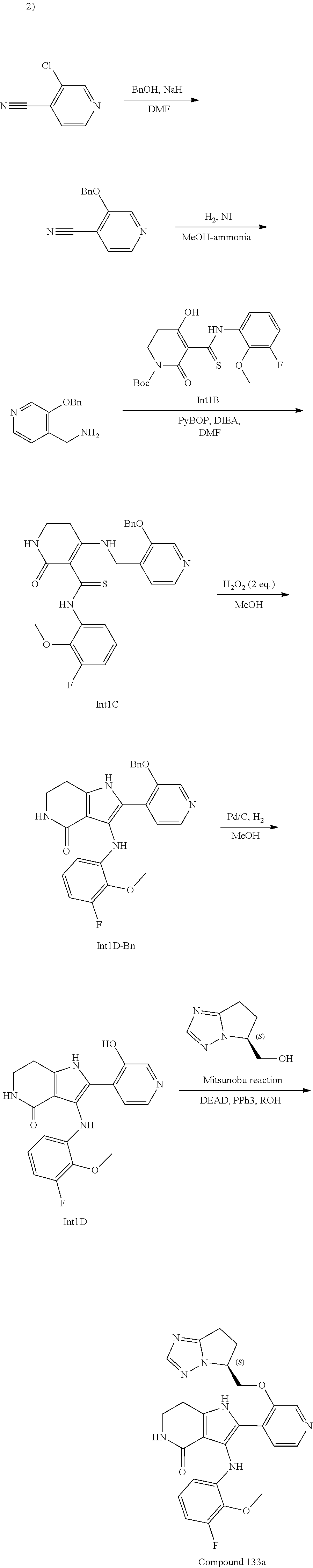

### Part 1: Synthesis of Int1B

3-Fluoro-2-methoxyaniline is reacted with CSCl₂ to provide Int1A. The coupling of Int1A with tert-butyl 4-hydroxy-6-oxo-3,6-dihydropyridine-1 (2H)-carboxylate provides Int1B.

### Part 2: Synthesis of Compound 133a

3-Chloroisonicotinonitrile is coupled with benzyl alcohol (BnOH) under basic conditions (e.g., NaH) in a polar aprotic solvent such as dimethylformamide (DMF) to provide 3-(benzyloxy)isonicotinonitrile. The nitrile reduction of 3-(benzyloxy)isonicotinonitrile in the presence of catalytic nickel and hydrogen gas (H2) provides (3-(benzyloxy)pyridin-4-yl)methanamine. The coupling Int1B and (3-(benzyloxy)pyridin-4-yl)methanamine provides Int1C. Cyclization of Int1C under oxidative conditions (e.g., in the presence of H₂O₂) then provides Int1D-Bn. Benzyl deproction under hydrogenative conditions (e.g., Pd/C and H2) provides Int1D. The Mitsunobu coupling of Int1D with (S)-(6,7-dihydro-5H-pyrrolo[1,2-b][1,2,4]triazol-5-yl)methanol affords Compound 133a.

### Example 2: 2-(3-((6,7-dihydro-5H-pyrrolo[1,2-a]imidazol-3-yl)methoxy)pyridin-4-yl)-3-((3-fluoro-2-methoxyphenyl)amino)-1,5,6,7-tetrahydro-4H-pyrrolo[3,2-c]pyridin-4-one (Compound 150)

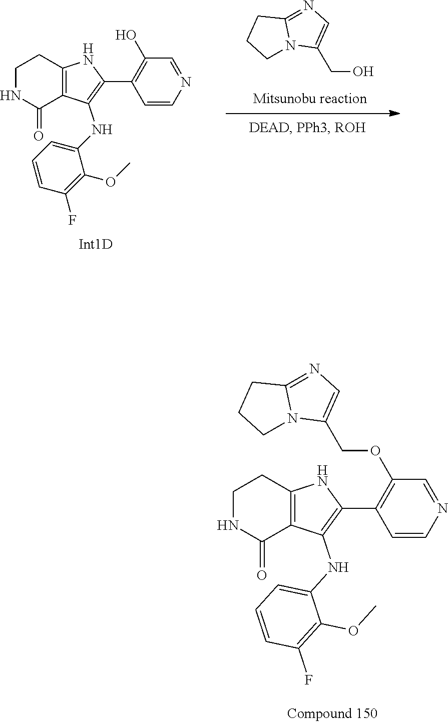 [SMILES: COc1c(F)cccc1Nc1c(-c2ccncc2O)[nH]c2c1C(=O)NCC2 ; OCc1cnc2n1CCC2 ; COc1c(F)cccc1Nc1c(-c2ccncc2OCc2cnc3n2CCC3)[nH]c2c1C(=O)NCC2]

Int1D is prepared using the method described in Example 1. The Mitsunobu coupling of Int1D with (6,7-dihydro-5H-pyrrolo[1,2-a]imidazol-3-yl)methanol affords Compound 150.

### Example 3: 2-(3-((6,7-dihydro-5H-pyrrolo[1,2-a]imidazol-5-yl)methoxy)pyridin-4-yl)-3-((3-fluoro-2-methoxyphenyl)amino)-1,5,6,7-tetrahydro-4H-pyrrolo[3,2-c]pyridin-4-one (Compound 128)

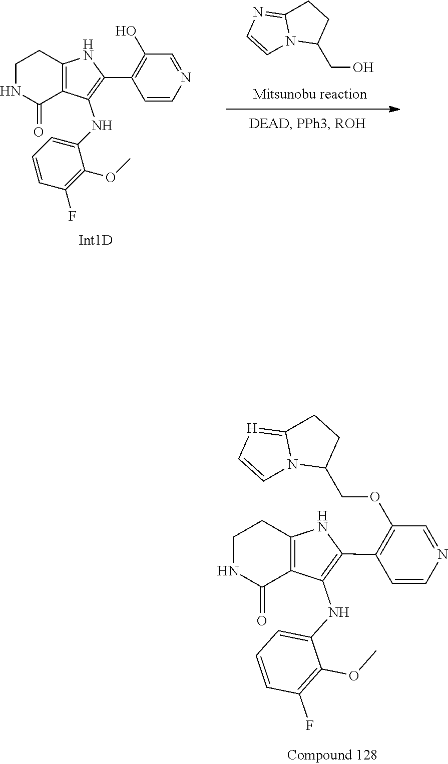

Int1D is prepared using the method described in Example 1. The Mitsunobu coupling of Int1D with (6,7-dihydro-5H-pyrrolo[1,2-a]imidazol-5-yl)methanol affords Compound 128.

### Example 4: 3-((3-fluoro-2-methoxyphenyl)amino)-2-(3-((1,4,5,6-tetrahydrocyclopenta[c] pyrazol-3-yl)methoxy)pyridin-4-yl)-1,5,6,7-tetrahydro-4H-pyrrolo[3,2-c]pyridin-4-one (Compound 151)

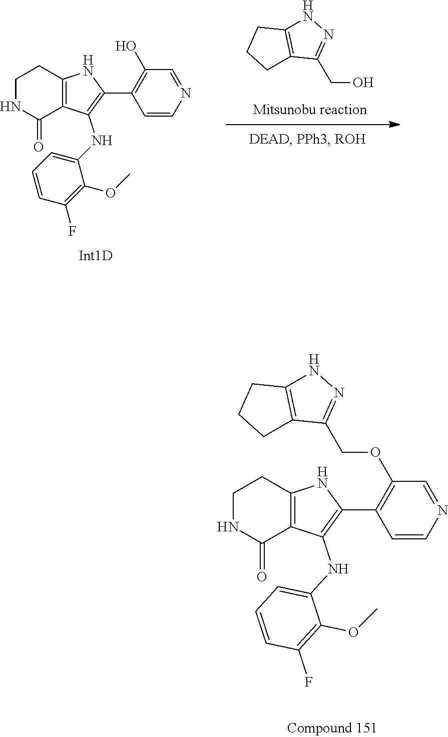 [SMILES: COc1c(F)cccc1Nc1c(-c2ccncc2O)[nH]c2c1C(=O)NCC2 ; OCc1n[nH]c2c1CCC2 ; COc1c(F)cccc1Nc1c(-c2ccncc2OCc2n[nH]c3c2CCC3)[nH]c2c1C(=O)NCC2]

Int1D is prepared using the method described in Example 1. The Mitsunobu coupling of Int1D with (1,4,5,6-tetrahydrocyclopenta[c]pyrazol-3-yl)methanol affords Compound 151.

### Example 5: Synthesis of (S)-3-((3-chloro-2-methoxyphenyl)amino)-2-(3-((1-(methylsulfonyl)pyrrolidin-2-yl)methoxy)pyridin-4-yl)-1,5,6,7-tetrahydro-4H-pyrrolo[3,2-c]pyridin-4-one (Compound 142a)

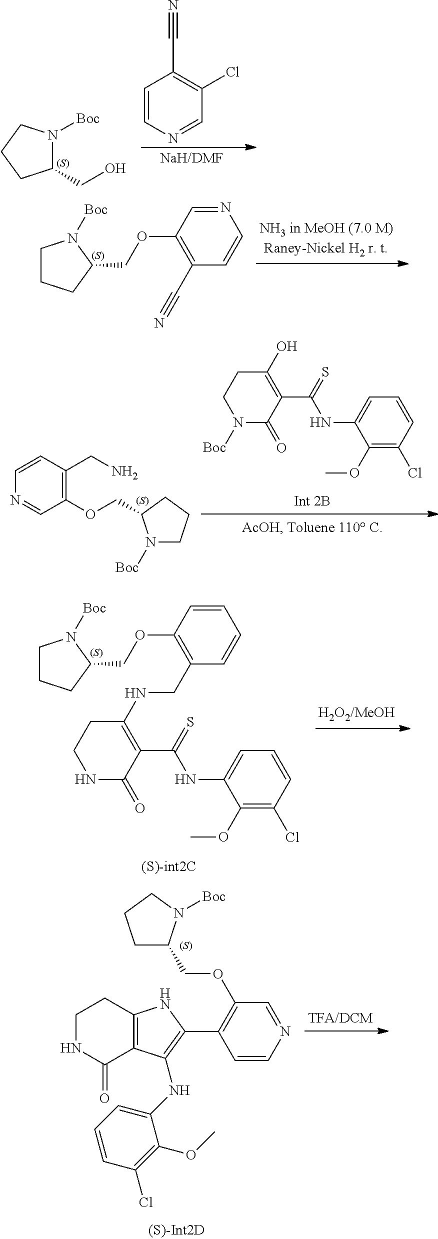

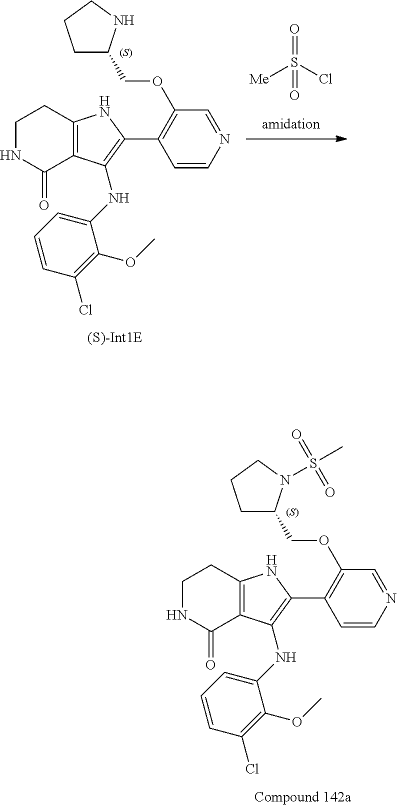

Tert-butyl (S)-2-(hydroxymethyl)pyrrolidine-1-carboxylate is coupled with 3-chloroisonicotinonitrile under basic conditions (e.g., in the presence of NaH) in a polar, aprotic solvent such as dimethylformamide (DMF) to provide tert-butyl (S)-2-(((4-cyanopyridin-3-yl)oxy)methyl)pyrrolidine-1-carboxylate. The nitrile reduction of (S)-2-(((4-cyanopyridin-3-yl)oxy)methyl)pyrrolidine-1-carboxylate (e.g., in the presence of Raney nickel and hydrogen gas) then provides tert-butyl (S)-2-(((4-(aminomethyl)pyridin-3-yl)oxy)methyl)pyrrolidine-1-carboxylate. The coupling Int2B and tert-butyl (S)-2-(((4-(aminomethyl)pyridin-3-yl)oxy)methyl)pyrrolidine-1-carboxylate then provides (S)-Int2C. Cyclization of (S)-Int2C under oxidative conditions (e.g., in the presence of H₂O₂) then provides (S)-Int2D. BOC-deprotection of (S)-Int2D in the presence of acid (e.g., TFA, etc. e.g, in dichloromethane (DCM)) provides (S)-Int1E. Finally, sulfonamide formation of (S)-Int1E in the presence of methanesulfonyl chloride affords Compound 142a.

### Example 6: (S)-3-((3-chloro-2-methoxyphenyl)amino)-2-(3-((1-propionylpyrrolidin-2-yl)methoxy)pyridin-4-yl)-1,5,6,7-tetrahydro-4H-pyrrolo[3,2-c]pyridin-4-one (Compound 139)

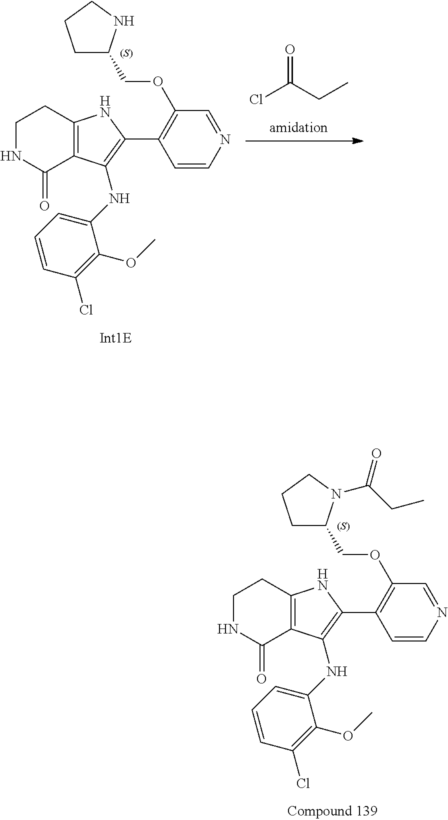

Int1E is prepared using the method described in Example 5. Amidation of Int1E in the presence of propionyl chloride affords Compound 139.

### Example 7: (S)-2-(3-((1-acryloylpyrrolidin-2-yl)methoxy)pyridin-4-yl)-3-((3-chloro-2-methoxyphenyl)amino)-1,5,6,7-tetrahydro-4H-pyrrolo[3,2-c]pyridin-4-one (Compound 138b)

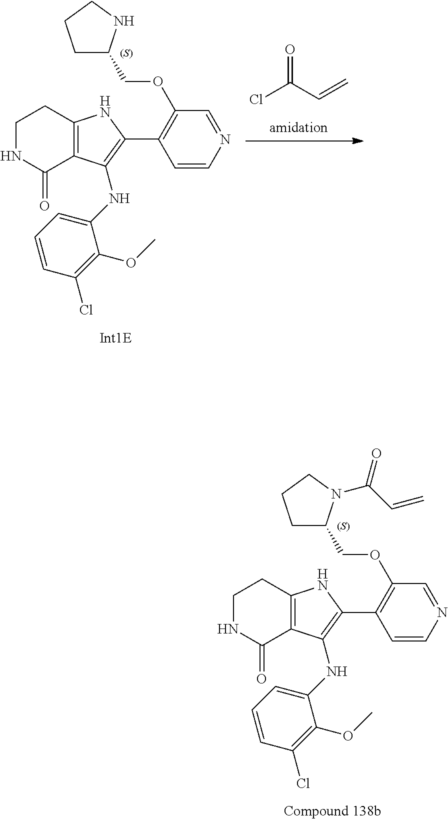

Int1E is prepared using the method described in Example 5. Amidation of Int1E in the presence of acryloyl chloride affords Compound 138b.

### Example 8: Synthesis of (S)-2-(3-((1-acetylpyrrolidin-2-yl)methoxy)pyridin-4-yl)-3-((3-chloro-2-methoxyphenyl)amino)-1,5,6,7-tetrahydro-4H-pyrrolo[3,2-c]pyridin-4-one (Compound 141a)

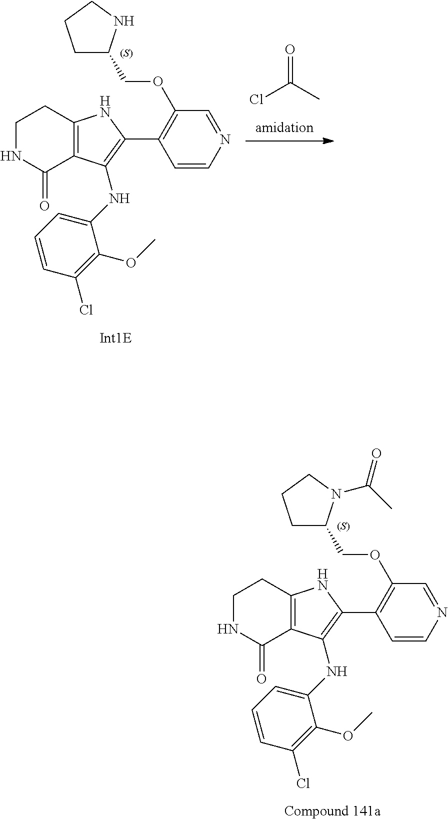

Int1E is prepared using the method described in Example 5. Amidation of Int1E in the presence of acetyl chloride affords Compound 141a.

### Example 9: (R)-2-(3-((1-acryloylpyrrolidin-2-yl)methoxy)pyridin-4-yl)-3-((3-fluoro-2-methylphenyl)amino)-1,5,6,7-tetrahydro-4H-pyrrolo[3,2-c]pyridin-4-one (Compound 140

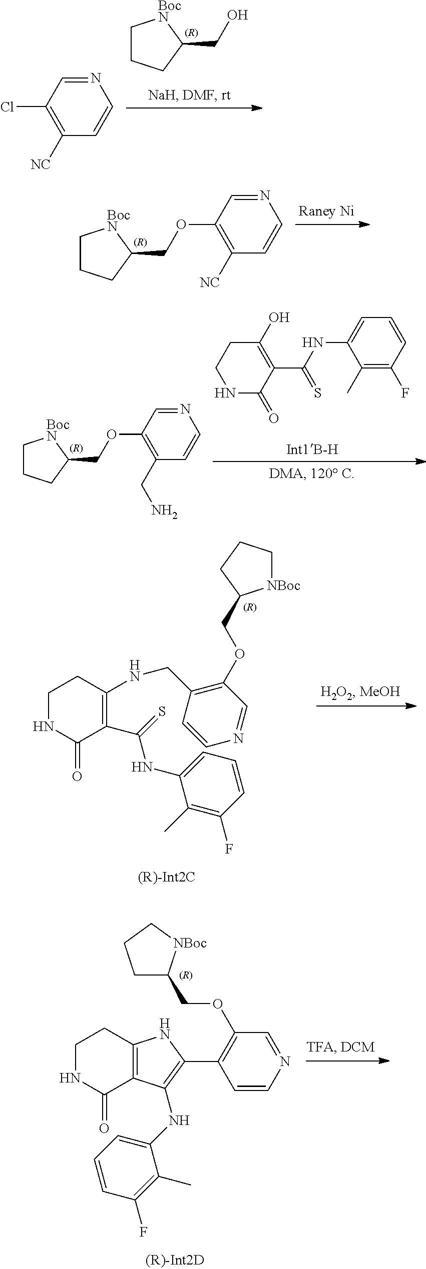

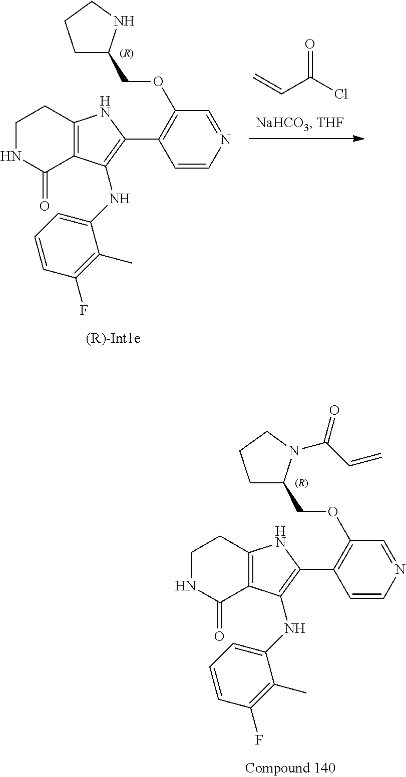

Tert-butyl (R)-2-(hydroxymethyl)pyrrolidine-1-carboxylate is coupled with 3-chloroisonicotinonitrile under basic conditions (e.g., in the presence of NaH) in a polar, aprotic solvent such as dimethylformamide (DMF) to provide tert-butyl (R)-2-(((4-cyanopyridin-3-yl)oxy)methyl)pyrrolidine-1-carboxylate. The nitrile reduction of (R)-2-(((4-cyanopyridin-3-yl)oxy)methyl)pyrrolidine-1-carboxylate (e.g., in the presence of Raney nickel and hydrogen gas) then provides tert-butyl (R)-2-(((4-(aminomethyl)pyridin-3-yl)oxy)methyl)pyrrolidine-1-carboxylate. The coupling Int1′B-H (see method for preparation below) and tert-butyl (R)-2-(((4-(aminomethyl)pyridin-3-yl)oxy)methyl)pyrrolidine-1-carboxylate then provides (R)-Int2C. Cyclization of (R)-Int2C under oxidative conditions (e.g., in the presence of H₂O₂) then provides (R)-Int2D. BOC-deprotection of (R)-Int2D in the presence of acid (e.g., TFA, etc. in e.g., dichloromethane (DCM)) provides (R)-Int1E. Amidation of (R)-Int1E in the presence of methanesulfonyl chloride affords Compound 140.

### Preparation of Int1′B-H:

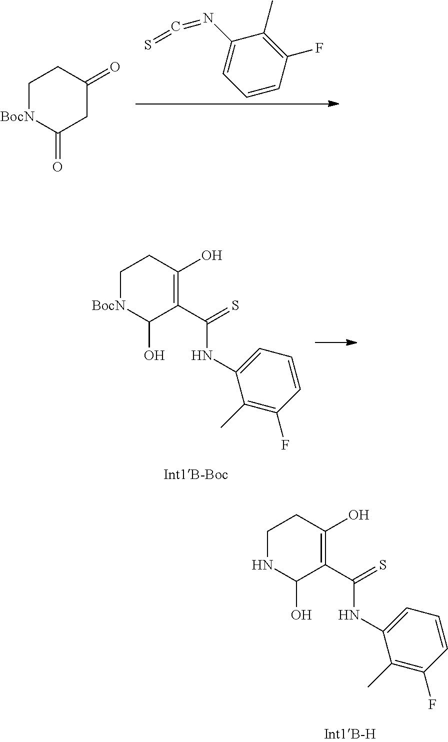

tert-butyl 2,4-dioxopiperidine-1-carboxylate is coupled with 1-fluoro-3-isothiocyanato-2-methylbenzene (e.g., in the presence of a base such as DBU in e.g., acetonitrile) provides Int1′B-Boc. Removal of the Boc protecting group on Int1′B-Boc then provides Int1′B-H.

### Example 10: (R)-2-(3-((1-acryloylazetidin-2-yl)methoxy)pyridin-4-yl)-3-((3-chloro-2-methoxyphenyl)amino)-1,5,6,7-tetrahydro-4H-pyrrolo[3,2-c]pyridin-4-one (Compound 145a)

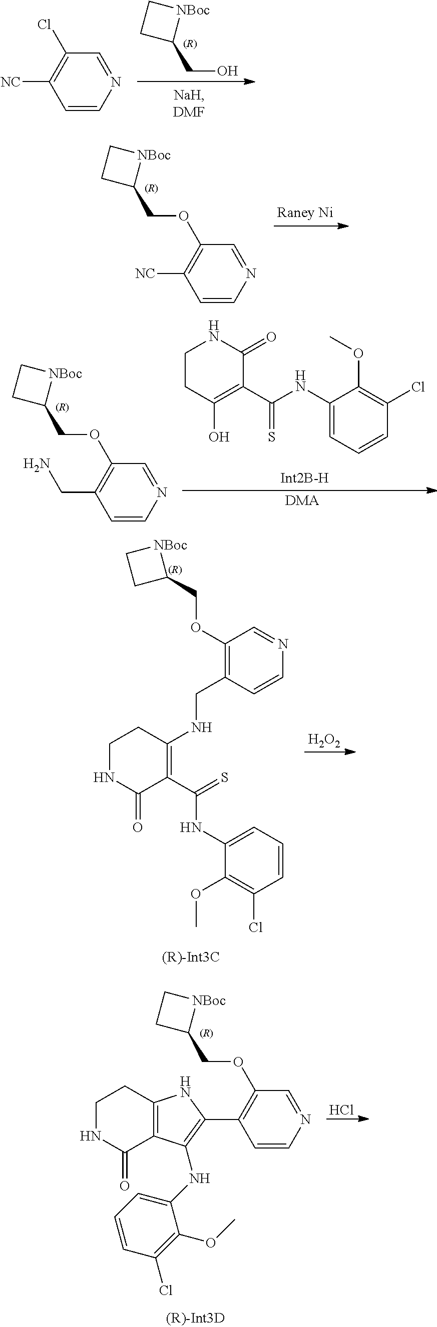

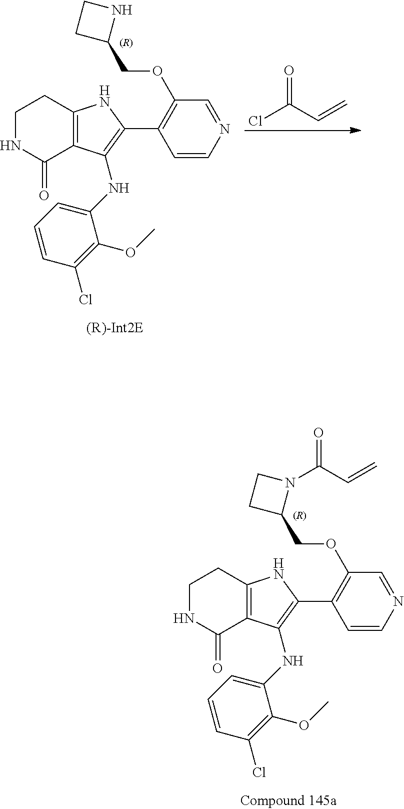

Tert-butyl (R)-2-(hydroxymethyl)azetidine-1-carboxylate is coupled with 3-chloroisonicotinonitrile under basic conditions (e.g., in the presence of NaH) in a polar, aprotic solvent such as dimethylformamide (DMF) to provide tert-butyl (R)-2-(((4-cyanopyridin-3-yl)oxy)methyl)azetidine-1-carboxylate. The nitrile reduction of tert-butyl (R)-2-(((4-cyanopyridin-3-yl)oxy)methyl)azetidine-1-carboxylate (e.g., in the presence of Raney nickel and hydrogen gas) then provides tert-butyl (R)-2-(((4-(aminomethyl)pyridin-3-yl)oxy)methyl)azetidine-1-carboxylate. The coupling Int2B-H and tert-butyl (R)-2-(((4-(aminomethyl)pyridin-3-yl)oxy)methyl)pyrrolidine-1-carboxylate then provides (R)-Int3C. Cyclization of (R)-Int3C under oxidative conditions (e.g., in the presence of H₂O₂) then provides (R)-Int3D. BOC-deprotection of (R)-Int3D in the presence of acid (e.g., TFA, etc.) in dichloromethane (DCM) provides (R)-Int2E. Finally, amidation of (R)-Int2E in the presence of acyloyl chloride affords Compound 145a.

### Example 11. Synthesis of 2-(3-(2-cyclopropoxyethoxy)pyridin-4-yl)-3-((3-fluoro-2-methoxyphenyl)amino)-1,5,6,7-tetrahydro-4H-pyrrolo[3,2-c]pyridin-4-one (Compound 135)

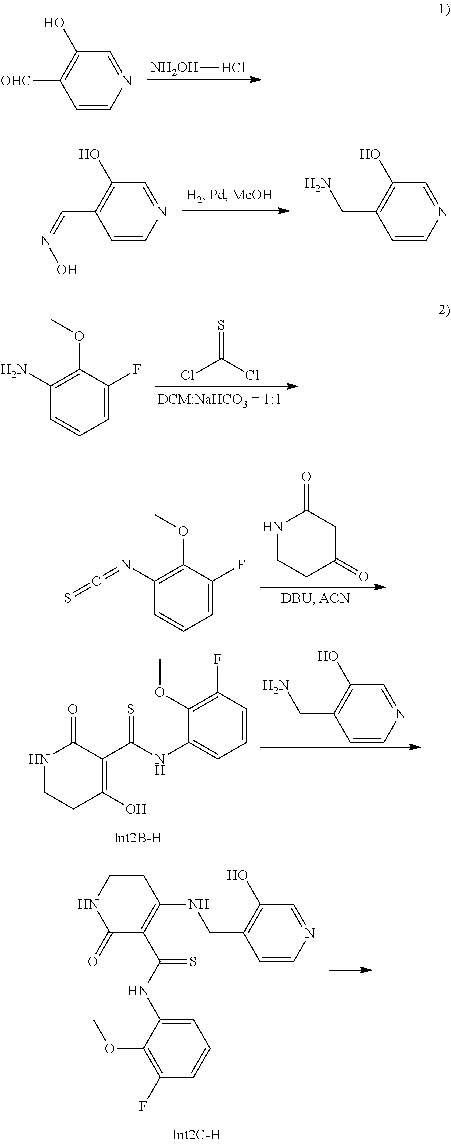

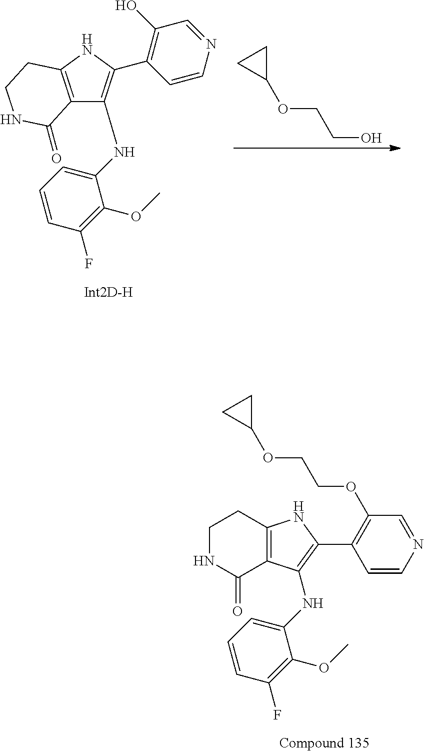

### Part 1: Synthesis of 4-(aminomethyl)pyridin-3-ol

3-Hydroxyisonicotinaldehyde is reacted with hydroxylamine hydrochloride (NH₂OH·HCl) to provide 3-hydroxyisonicotinaldehyde oxime. Hyrdogenation of the imine of 3-hydroxyisonicotinaldehyde oxime (e.g., in the presence of palladium and H₂) in methanol provides 4-(aminomethyl)pyridin-3-ol

### Part 2

3-Fluoro-2-methoxyaniline is reacted with CSCl₂ to provide 1-fluoro-3-isothiocyanato-2-methoxybenzene. The coupling of 1-fluoro-3-isothiocyanato-2-methoxybenzene and piperidine-2,4-dione (e.g., in the presence of DBU) in acetonitrile provides Int2B-H. The coupling of Int2B-H with 4-(aminomethyl)pyridin-3-ol provides Int2C-H. Cyclization of Int2C-H under oxidative conditions (e.g., in the presence of H₂O₂) then provides Int2D-H. The Mitsunobu coupling of Int2D-H with 2-cyclopropoxyethan-1-ol affords Compound 135.

### Example 12: Synthesis of 3-((3-fluoro-2-methoxyphenyl)amino)-2-(3-((1R,2S)-2-methoxycyclopropoxy)pyridin-4-yl)-1,5,6,7-tetrahydro-4H-pyrrolo[3,2-c]pyridin-4-one (Compound 136b)

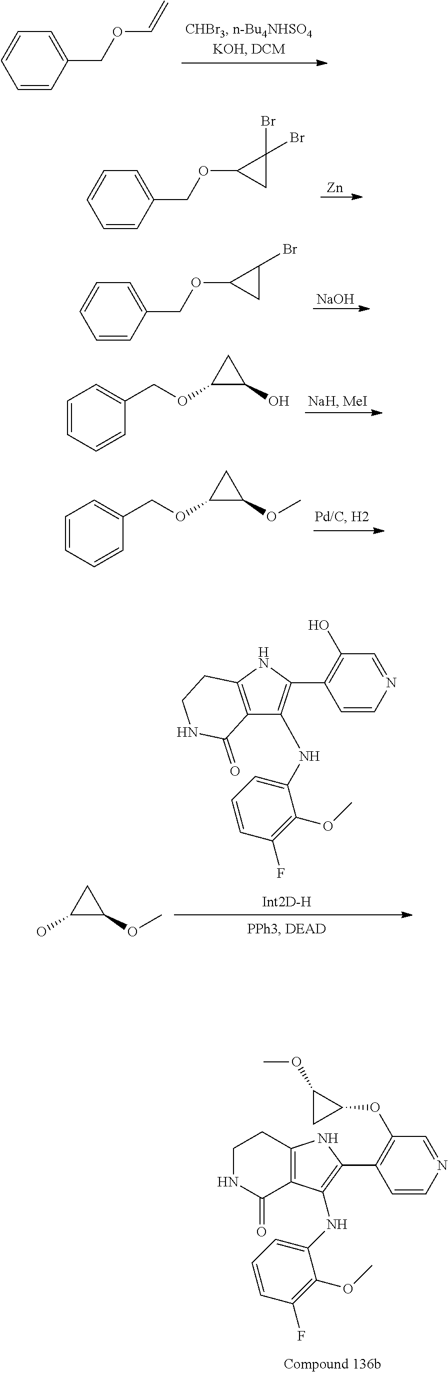

((Vinyloxy)methyl)benzene is reacted with bromoform (CHBr₃) under basic conditions (e.g., in the presences of KOH and n-Bu₄NHSO4 in DCM) to provide ((2,2-dibromocyclopropoxy)methyl)benzene. Sequential debromination of ((2,2-dibromocyclopropoxy)methyl)benzene under reductive conditions (e.g., with zinc) followed by basic hydrolysis (e.g., in the presence of NaOH) provides (1R,2R)-2-(benzyloxy)cyclopropan-1-ol. Methylation of (1R,2R)-2-(benzyloxy)cyclopropan-1-ol (e.g., in the presence of NaH and Mel) provides (((1R,2R)-2-methoxycyclopropoxy)methyl)benzene. Hydrogenolysis of (((1R,2R)-2-methoxycyclopropoxy)methyl)benzene followed by Mitsunobu coupling with Int2D-H affords Compound 136b.

### Example 13: Synthesis of (R)-2-(3-((1-acryloylpyrrolidin-2-yl)ethynyl)pyridin-4-yl)-3-((3-fluoro-2-methoxyphenyl)amino)-1,5,6,7-tetrahydro-4H-pyrrolo[3,2-c]pyridin-4-one (Compound 147b)

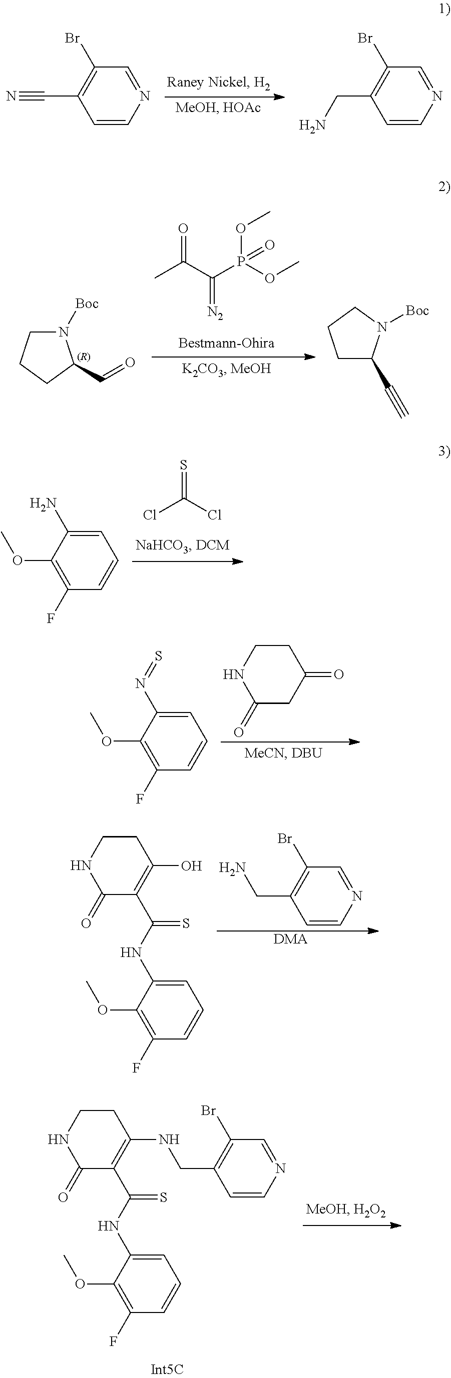

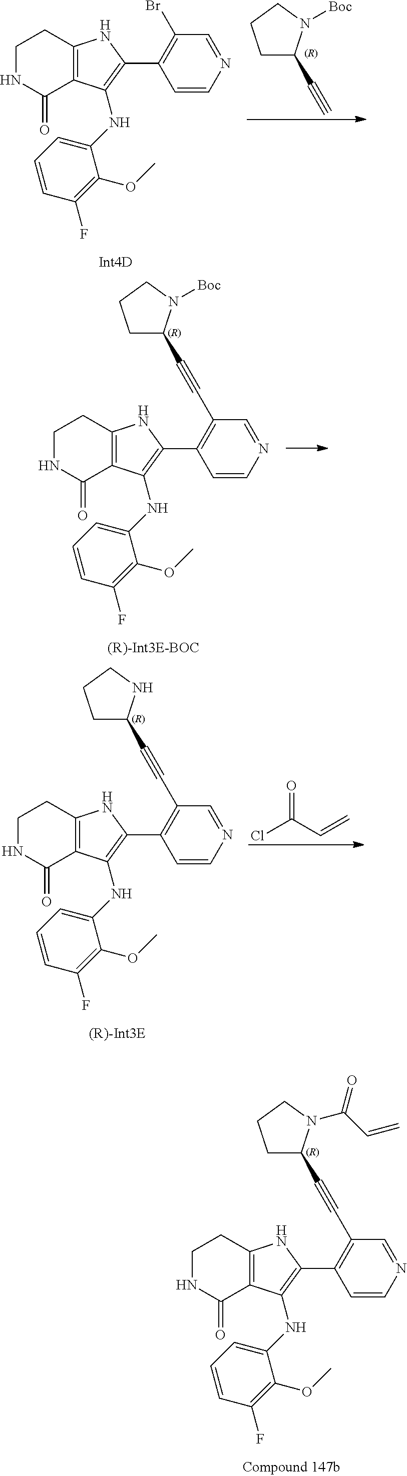

### Part 1: Synthesis of (3-bromopyridin-4-yl)methanamine

3-Bromoisonicotinonitrile is reduced under hydrogenative conditions (e.g., Raney nickel and H2) in acetic acid to provide (3-bromopyridin-4-yl)methanamine.

### Part 2: Synthesis of tert-butyl (R)-2-ethynylpyrrolidine-1-carboxylate

Tert-butyl (R)-2-formylpyrrolidine-1-carboxylate is reacted (e.g., in the presence of Bestmann-Ohira reagent and K₂CO₃) to provide tert-butyl (R)-2-ethynylpyrrolidine-1-carboxylate.

### Part 3

3-Fluoro-2-methoxyaniline is reacted with CSCl₂under basic conditions (e.g., NaHCO₃ in DCM) to provide 1-fluoro-3-isothiocyanato-2-methoxybenzene. Coupling of 1-fluoro-3-isothiocyanato-2-methoxybenzene and piperidine-2,4-dione provides N-(3-fluoro-2-methoxyphenyl)-4-hydroxy-2-oxo-1,2,5,6-tetrahydropyridine-3-carbothioamide. The reaction of N-(3-fluoro-2-methoxyphenyl)-4-hydroxy-2-oxo-1,2,5,6-tetrahydropyridine-3-carbothioamide with (3-bromopyridin-4-yl)methanamine in a polar aprotic solvent (e.g., DMA) provides Int5C. Cyclization of Int5C under oxidative conditions (e.g., in the presence of H₂O₂) then provides Int4D. Tert-butyl (R)-2-ethynylpyrrolidine-1-carboxylate is coupled with Int4D to provide (R)-Int3E-BOC. BOC-deprotection of (R)-Int3E-BOC provides (R)-Int3E, which is further reacted with acryloyl chloride to afford Compound 147b.

### Example 14: Synthesis of (S)-2-(3-((1-acryloylpyrrolidin-2-yl)ethynyl)pyridin-4-yl)-3-((3-fluoro-2-methoxyphenyl)amino)-1,5,6,7-tetrahydro-4H-pyrrolo[3,2-c]pyridin-4-one (Compound 147a)

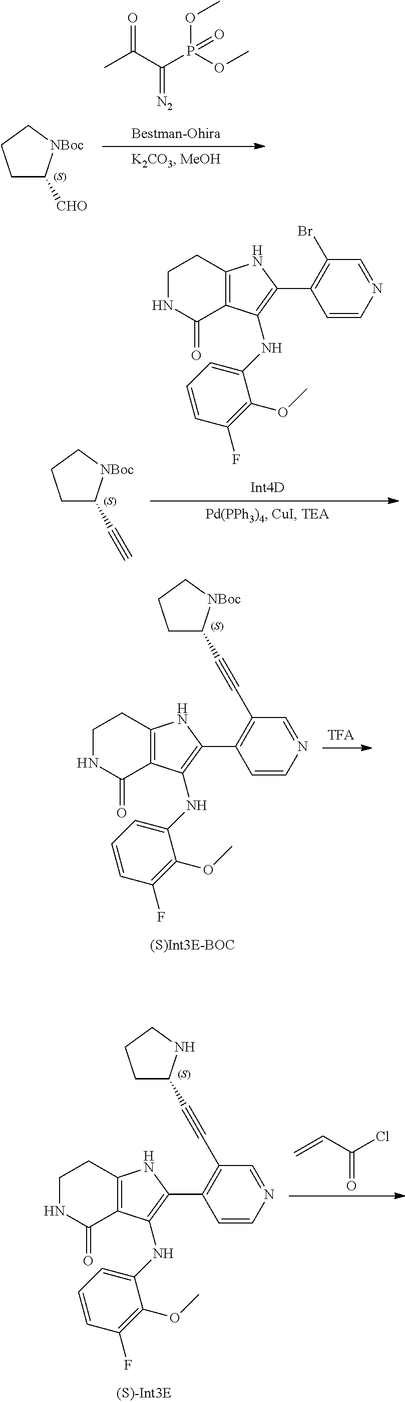

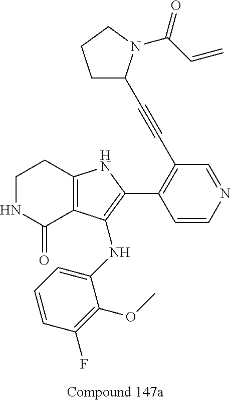 [SMILES: C=CC(=O)N1CCCC1C#Cc1cnccc1-c1[nH]c2c(c1Nc1cccc(F)c1OC)C(=O)NCC2]

Tert-butyl (S)-2-formylpyrrolidine-1-carboxylate is reacted (e.g., in the presence of Bestmann-Ohira reagent and K₂CO₃) to provide tert-butyl (S)-2-ethynylpyrrolidine-1-carboxylate. Sonogashira coupling of Int4D and tert-butyl (S)-2-ethynylpyrrolidine-1-carboxylate (e.g., in the presence of Pd(PPh₃)₄, CuI, and TEA) provides (S)-Int3E-BOC. BOC-deprotection of (R)-Int3E-BOC provides (R)-Int3E, and is further reacted with acryloyl chloride to afford Compound 147a.

### Example 15: Synthesis of (S)-3-((3-fluoro-2-methoxyphenyl)amino)-2-(3-((tetrahydrofuran-2-yl)ethynyl)pyridin-4-yl)-1,5,6,7-tetrahydro-4H-pyrrolo[3,2-c]pyridin-4-one (Compound 148a) and (R)-3-((3-fluoro-2-methoxyphenyl)amino)-2-(3-((tetrahydrofuran-2-yl)ethynyl)pyridin-4-yl)-1,5,6,7-tetrahydro-4H-pyrrolo[3,2-c]pyridin-4-one (Compound 148b)

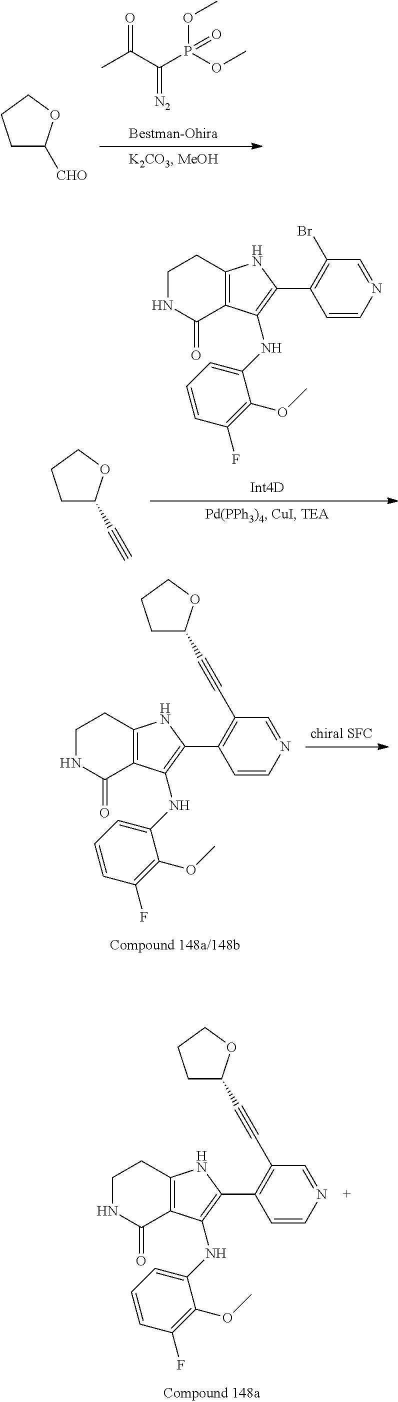

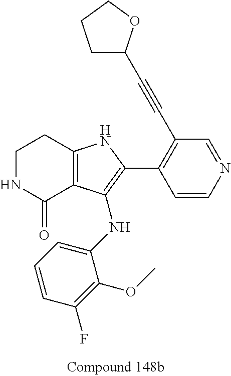 [SMILES: COc1c(F)cccc1Nc1c(-c2ccncc2C#CC2CCCO2)[nH]c2c1C(=O)NCC2]

Tetrahydrofuran-2-carbaldehyde is reacted (e.g., in the presence of the Bestmann-Ohira reagent and K₂CO₃) to provide 2-ethynyltetrahydrofuran. Sonogashira coupling of Int4D and 2-ethynyltetrahydrofuran (e.g., in the presence of Pd(PPh₃)₄, CuI, and TEA) provides an enantiomeric mixture comprising 148a and 148b, which is then separated by chiral supercritical fluid chromatography to afford Compound 148a and Compound 148b.

### Example 16: 3-((3-chloro-2-methoxyphenyl)amino)-2-(3-(3-methoxy-3-methylbut-1-yn-1-yl)pyridin-4-yl)-1,5,6,7-tetrahydro-4H-pyrrolo[3,2-c]pyridin-4-one (Compound 188)

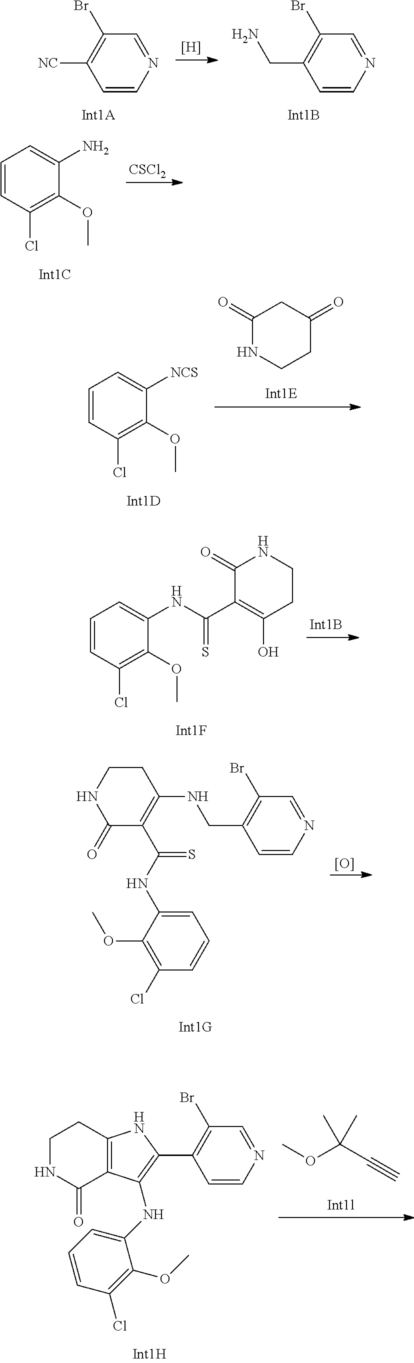

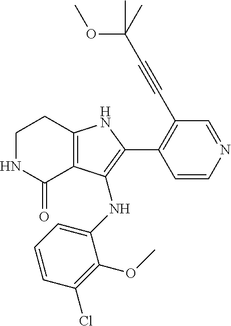 [SMILES: COc1c(Cl)cccc1Nc1c(-c2ccncc2C#CC(C)(C)OC)[nH]c2c1C(=O)NCC2]

Cyanopyridine Int1A is hydrogenated in the presence of hydrogen gas and a catalyst, e.g, Raney Ni in a polar protic solvent e.g., MeOH with mild acid, e.g., HOAc to give Int1B. Int1C is reacted with thiophosgene under modified Schotten-Baumann conditions, e.g., NaHCO₃ in the presence of water/DCM to give the corresponding thioisocyanate Int1D. Treatment of Int1D with Int1E in the presence of a strong base, e.g., DBU in a polar aprotic solvent, e.g., ACN gives Int1F. Condensation of Int1F with Int1B with heating, e.g., 120° C. in the presence of a dehydrating agent, e.g., 4A molecular sieves in a polar aprotic solvent, e.g., DMA provides Int1G. Oxidative cyclization of Int1G in the presence of a mild oxidant, e.g., H₂O₂ in and polar protic solvent, e.g., MeOH gives Int1H. Reaction of Int1H with Int1I under Sonogashira coupling conditions, e.g., CuI, Pd(PPh₃)₄, and TEA gives the title compound.

### Example 17: Synthesis of (R)-217-(3-((1-acryloylpyrrolidin-2-yl)methoxy)pyridin-4-yl)-3-(benzo[d]thiazol-4-ylamino)-1,5,6,7-tetrahydro-4H-pyrrolo[3,2-c]pyridin-4-one (Compound 171)

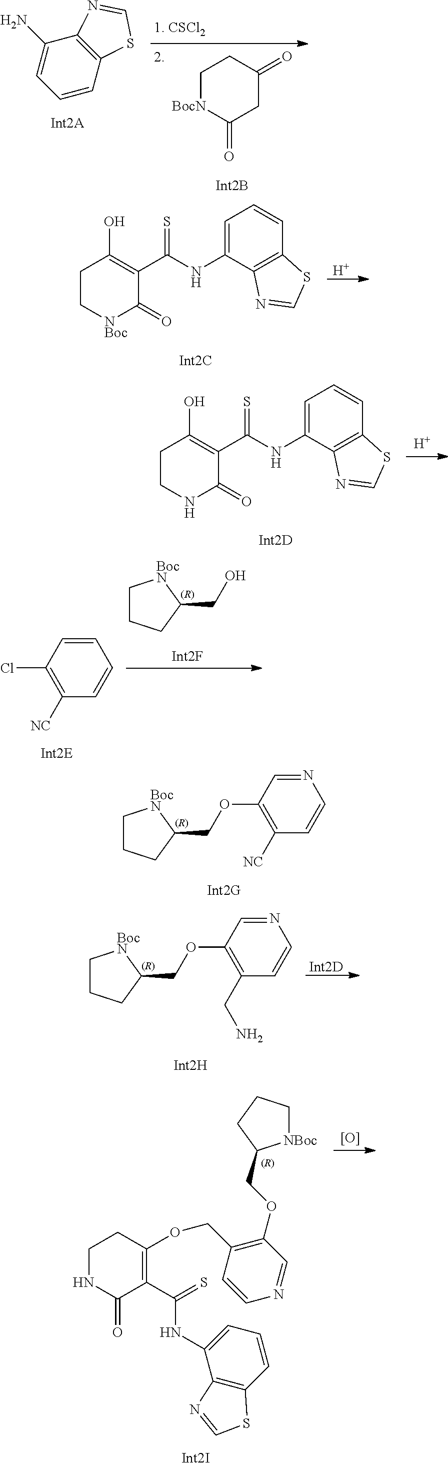

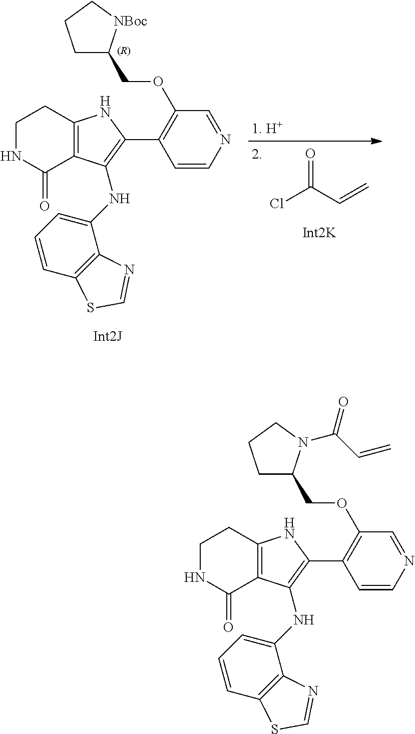

Aminobenzothiazole Int2A is reacted with thiophosgene under modified Schotten-Baumann conditions, e.g., NaHCO₃ in the presence of water/DCM to give the corresponding thioisocyanate, which is then reacted with Int2B in the presence of a strong base, e.g., DBU in a polar aprotic solvent, e.g., ACN to give Int2C. Deprotection of Int2C is accomplished with strong acid, e.g., TFA in DCM followed by neutralization with NaHCO₃(aq) to give Int2D. Reaction of chloropyridine Int2E with Int2F in the presence of a strong base, e.g., NaH in a polar aprotic solvent, e.g., DMF at room temperature gives Int2G. Hydrogenation of Int2G to afford Int2H is accomplished with hydrogen gas and a catalyst, e.g, Raney Ni in a polar protic solvent e.g., MeOH. Condensation of Int2H with Int2D with heating, e.g., 120° C. in the presence of a dehydrating agent, e.g., 4A molecular sieves in a polar aprotic solvent, e.g., DMA provides Int2I. Oxidative cyclization of Int2I in the presence of a mild oxidant, e.g., H₂O₂ in and polar protic solvent, e.g., MeOH gives Int2J. Deprotection of Int2J with a strong acid, e.g., TFA followed by treatment with acryloyl chloride Int2K under mildly basic conditions gives the title compound.

### Example 18. 2-(3-[[(2R)-1-acetylpyrrolidin-2-yl]methoxy]pyridin-4-yl)-3-[(3-chloro-2-methoxyphenyl)amino]-1H,5H,6H,7H-pyrrolo[3,2-c]pyridin-4-one (compound 141b)

### 18.1. Synthesis of chloro-3-isothiocyanato-2-methoxybenzene

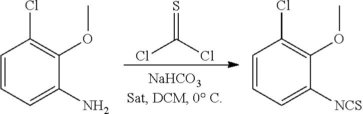

A solution of 3-chloro-2-methoxyaniline (4.00 g, 25.3 mmol, 1.00 equiv) and thiophosgene (3.21 g, 27.9 mmol, 1.10 equiv) in DCM (10.00 mL) and NaHCO₃Sat (10.00 mL) was stirred for 1 h at 0° C. under nitrogen atmosphere. The resulting mixture was extracted with CH2Cl2 (2×100 mL). The combined organic layers were washed with brine (1×50 mL), dried over anhydrous Na2SO4. After filtration, the filtrate was concentrated under reduced pressure. This resulted in 1-chloro-3-isothiocyanato-2-methoxybenzene (3.5 g, 69.07%) as a light yellow oil.

LC-MS: (M+H)+ found: 200.0.

### 18.2. Synthesis of tert-butyl 3-[(3-chloro-2-methoxyphenyl)carbamothioyl]-4-hydroxy-2-oxo-5,6-dihydropyridine

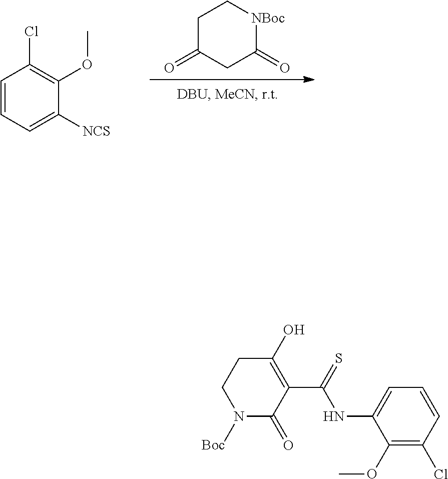

A solution of 1-chloro-3-isothiocyanato-2-methoxybenzene (3.50 g, 17.5 mmol, 1.00 equiv) and tert-butyl 2,4-dioxopiperidine-1-carboxylate (3.74 g, 17.531 mmol, 1.00 equiv) and DBU (4.00 g, 26.296 mmol, 1.50 equiv) in MeCN (50.00 mL) was stirred for 2h at room temperature under nitrogen atmosphere. The reaction was quenched by the addition of Water (100 mL) at room temperature. The mixture was acidified to pH 7 with conc. HCl. The precipitated solids were collected by filtration and washed with water (1×10 mL). This resulted in tert-butyl 3-[(3-chloro-2-methoxyphenyl)carbamothioyl]-4-hydroxy-2-oxo-5,6-dihydropyridine-1-carboxylate (6.9 g, 95.33%) as a yellow solid.

LC-MS: (M+H)⁺ found 413.3.

### 18.3. Synthesis of tert-butyl (2R)-2-[[(4-cyanopyridin-3-yl)oxy]methyl]pyrrolidine-1-carboxylate

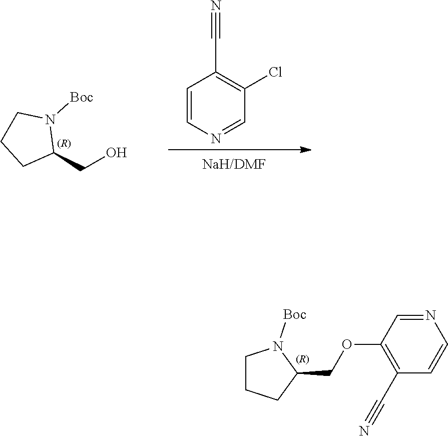

To a solution of tert-butyl (2R)-2-(hydroxymethyl)pyrrolidine-1-carboxylate (5.00 g, 24.8 mmol, 1.00 equiv) in DMF (50.00 mL) was added NaH (596 mg, 24.8 mmol, 1.00 equiv), stirred for 0.5h at 0° C., and 3-chloropyridine-4-carbonitrile (3.79 g, 27.3 mmol, 1.10 equiv) was added, stirred for 2h at 0° C. under nitrogen atmosphere. The reaction was quenched with Water at room temperature. The resulting mixture was extracted with EtOAc (3×50 mL). The combined organic layers were washed with brine (1×50 mL), dried over anhydrous Na2SO4. After filtration, the filtrate was concentrated under reduced pressure. The residue was purified by silica gel column chromatography, eluted with PE/EtOAc (5:1) to afford tert-butyl (2R)-2-[[(4-cyanopyridin-3-yl)oxy]methyl]pyrrolidine-1-carboxylate (3.2 g, 42.46%) as a light yellow oil.

LC-MS: (M-56)⁺ found 248.2.

### 18.4. Synthesis of tert-butyl (2R)-2-([[4-(aminomethyl)pyridin-3-yl]oxy]methyl)pyrrolidine-1-carboxylate

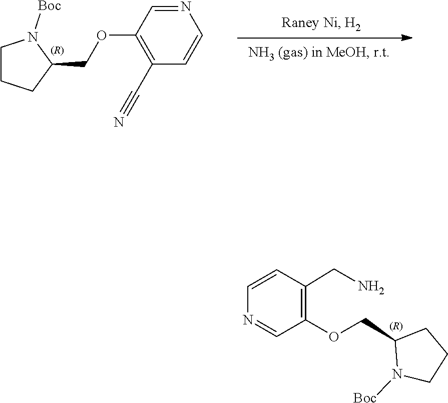

A solution of tert-butyl (2R)-2-[[(4-cyanopyridin-3-yl)oxy]methyl]pyrrolidine-1-carboxylate (6.00 g, 19.7 mmol, 1.00 equiv) and Raney Ni (2.54 g, 29.6 mmol, 1.50 equiv) and NH₃ (30 mL, 7M in MeOH) in MeOH (60.00 mL) was stirred for overnight at room temperature under hydrogen atmosphere. The resulting mixture was filtered, the filter cake was washed with MeOH (2×50 mL). The filtrate was concentrated under reduced pressure. The residue was purified by reverse phase flash with the following conditions (MeCN/H₂O=10%) to afford tert-butyl (2R)-2-([[4-(aminomethyl)pyridin-3-yl]oxy]methyl)pyrrolidine-1-carboxylate (5.0 g, 82.24%) as a light yellow oil.

LC-MS: (M+H)⁺ found 308.2.

### 18.5. Synthesis of tert-butyl 4-[[(3-[[(2R)-1-(tert-butoxycarbonyl)pyrrolidin-2-yl]methoxy]pyridin-4-yl)methyl]amino]-3-[(3-chloro-2-methoxyphenyl)carbamothioyl]-2-oxo-5,6-dihydropyridine-1-carboxylate

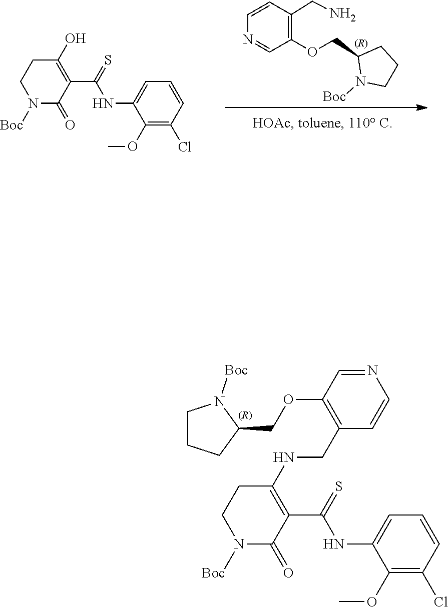

A solution of tert-butyl 3-[(3-chloro-2-methoxyphenyl)carbamothioyl]-4-hydroxy-2-oxo-5,6-dihydropyridine-1-carboxylate (1.50 g, 3.6 mmol, 1.00 equiv) and tert-butyl (2R)-2-([[4-(aminomethyl)pyridin-3-yl]oxy]methyl)pyrrolidine-1-carboxylate (1.11 g, 3.6 mmol, 1.00 equiv), HOAc (218 mg, 3.6 mmol, 1.00 equiv) in Toluene (20.00 mL) was stirred for 1 h at 110° C. under nitrogen atmosphere. The resulting mixture was concentrated under reduced pressure. The residue was purified by Prep-TLC (PE/EtOAc 2:1) to afford tert-butyl 4-[[(3-[[(2R)-1-(tert-butoxycarbonyl)pyrrolidin-2-yl]methoxy]pyridin-4-yl)methyl]amino]-3-[(3-chloro-2-methoxyphenyl)carbamothioyl]-2-oxo-5,6-dihydropyridine-1-carboxylate (1.4 g, 54.87%) as a light yellow solid.

LC-MS: (M+H)⁺ found 702.2.

### 18.6. Synthesis of tert-butyl (2R)-2-[([4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl]oxy)methyl]pyrrolidine-1-carboxylate

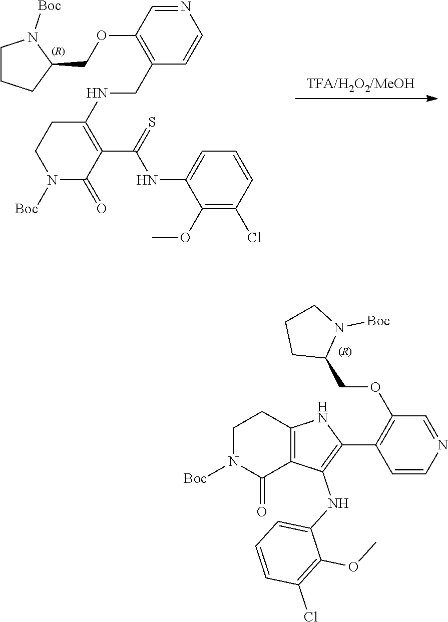

A solution of tert-butyl 4-[[(3-[[(2R)-1-(tert-butoxycarbonyl)pyrrolidin-2-yl]methoxy]pyridin-4-yl)methyl]amino]-3-[(3-chloro-2-methoxyphenyl)carbamothioyl]-2-oxo-5,6-dihydropyridine-1-carboxylate (1.40 g, 1.9 mmol, 1.00 equiv), TFA (454 mg, 3.987 mmol, 2.00 equiv) and H₂O₂ (30%)(271 mg, 7.9 mmol, 4.00 equiv) in MeOH (20.00 mL) was stirred for 1 h at 60° C. under nitrogen atmosphere. The resulting mixture was concentrated under reduced pressure. This resulted in tert-butyl (2R)-2-[([4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl]oxy)methyl]pyrrolidine-1-carboxylate (880 mg, 66.06%) as a light yellow solid.

LC-MS: (M-100)⁺ found 568.2.

### 18.7. Synthesis of 3-[(3-chloro-2-methoxyphenyl)amino]-2-[3-[(2R)-pyrrolidin-2-ylmethoxy]pyridin-4-yl]-1H,5H,6H,7H-pyrrolo[3,2-c]pyridin-4-one

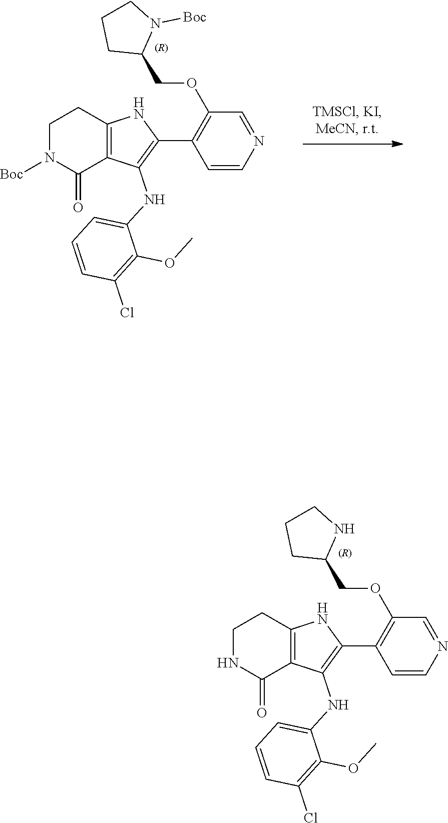

A solution of tert-butyl (2R)-2-[([4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl]oxy)methyl]pyrrolidine-1-carboxylate (750 mg, 1.1 mmol, 1.00 equiv) and TMSCl (487 mg, 4.490 mmol, 4.00 equiv), KI (745 mg, 4.4 mmol, 4.00 equiv) in MeCN (10.00 mL) was stirred for 1 h at room temperature under nitrogen atmosphere. The resulting mixture was concentrated under reduced pressure. The residue was purified by Prep-TLC (CH2Cl₂/MeOH 5:1) to afford 3-[(3-chloro-2-methoxyphenyl)amino]-2-[3-[(2R)-pyrrolidin-2-ylmethoxy]pyridin-4-yl]-1H,5H,6H,7H-pyrrolo[3,2-c]pyridin-4-one (230 mg, 43.79%) as a light yellow solid.

LC-MS: (M+H)⁺ found 468.1.

### 18.8. Synthesis of 2-(3-[[(2R)-1-acetylpyrrolidin-2-yl]methoxy]pyridin-4-yl)-3-[(3-chloro-2-methoxyphenyl)amino]-1H,5H,6H,7H-pyrrolo[3,2-c]pyridin-4-one

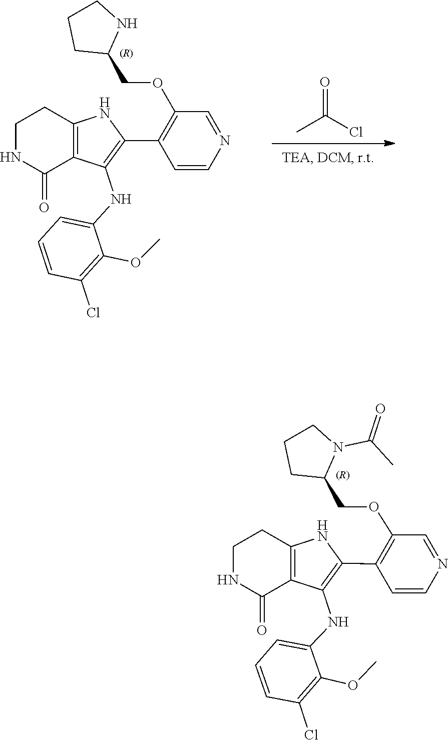

To a solution of 3-[(3-chloro-2-methoxyphenyl)amino]-2-[3-[(2R)-pyrrolidin-2-ylmethoxy]pyridin-4-yl]-1H,5H,6H,7H-pyrrolo[3,2-c]pyridin-4-one (60 mg, 0.1 mmol, 1.00 equiv) and TEA (25 mg, 0.2 mmol, 2.00 equiv) in DCM (1.00 mL) was added acetyl chloride (10 mg, 0.1 mmol, 1.00 equiv), stirred for 1 h at room temperature under nitrogen atmosphere. The resulting mixture was concentrated under reduced pressure. The crude product (mg) was purified by Prep-HPLC with the following conditions (Column: XBridge Prep OBD C18 Column, 30*150 mm, 5 μm; Mobile Phase A: Water (10 MMOL/L NH4HCO3), Mobile Phase B: ACN; Flow rate: 60 mL/min; Gradient: 25% B to 45% B in 8 min; Wave Length: 254 nm; RT1(min): 7.5;) to afford 2-(3-[[(2R)-1-acetylpyrrolidin-2-yl]methoxy]pyridin-4-yl)-3-[(3-chloro-2-methoxyphenyl)amino]-1H,5H,6H,7H-pyrrolo[3,2-c]pyridin-4-one (7.0 mg, 10.70%) as a yellow solid.

LC-MS: (M+H)⁺ found 510.1.

¹H NMR (400 MHz, DMSO-d₆) δ 11.47 (s, 1H), 8.39 (s, 1H), 7.97 (d, J=5.1 Hz, 1H), 7.42 (s, 1H), 7.33 (d, J=5.2 Hz, 1H), 7.11 (s, 1H), 6.66 (d, J=5.2 Hz, 2H), 6.10 (q, J=4.3, 3.7 Hz, 1H), 4.52 (s, 1H), 4.28 (dd, J=9.8, 7.0 Hz, 1H), 4.12 (dd, J=9.8, 4.3 Hz, 1H), 3.86 (s, 3H), 3.81 (s, 3H), 3.48 (d, J=7.9 Hz, 2H), 3.39 (d, J=2.6 Hz, 4H), 2.93 (s, 2H), 2.02 (s, 2H), 1.96 (d, J=38.6 Hz, 4H).

### Example 19. 3-[(3-chloro-2-methoxyphenyl)amino]-2-(3-[[(2R)-1-methanesulfonylpyrrolidin-2-yl]methoxy]pyridin-4-yl)-1H,5H,6H,7H-pyrrolo[3,2-c]pyridin-4-one (Compound 142b)

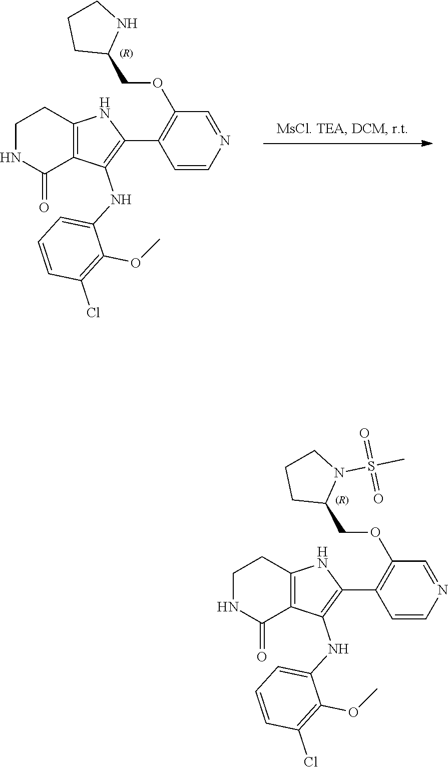

To a solution of 3-[(3-chloro-2-methoxyphenyl)amino]-2-[3-[(2R)-pyrrolidin-2-ylmethoxy]pyridin-4-yl]-1H,5H,6H,7H-pyrrolo[3,2-c]pyridin-4-one (60 mg, 0.1 mmol, 1.00 equiv) and TEA (25 mg, 0.2 mmol, 2.00 equiv) in DCM (1.00 mL) was added methanesulfonyl chloride (14 mg, 0.1 mmol, 1.00 equiv) at 0 room temperature, stirred for 1 h at room temperature under nitrogen atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: Water (0.1% FA), Mobile Phase B: ACN; Flow rate: 60 mL/min; Gradient: 15% B to 30% B in 8 min; Wave Length: 254 nm; RT1(min): 6;) to afford 3-[(3-chloro-2-methoxyphenyl)amino]-2-(3-[[(2R)-1-methanesulfonylpyrrolidin-2-yl]methoxy]pyridin-4-yl)-1H,5H,6H,7H-pyrrolo[3,2-c]pyridin-4-one (9.5 mg, 13.57%) as a yellow solid.

LC-MS: (M+H)+ found: 546.1.

¹H NMR (400 MHz, DMSO-d6) δ 11.06 (s, 1H), 8.40 (s, 1H), 8.02 (d, J=5.1 Hz, 1H), 7.42 (s, 1H), 7.30 (d, J=4.8 Hz, 1H), 7.11 (s, 1H), 6.70-6.60 (m, 2H), 6.13 (dd, J=7.0, 2.8 Hz, 1H), 4.24-4.09 (m, 3H), 3.60-3.35 (m, 4H), 2.97 (s, 3H), 2.84 (t, J=6.7 Hz, 2H), 2.01-1.87 (m, 4H).

### Example 20. 3-[(3-chloro-2-methoxyphenyl)amino]-2-(3-[[(2R)-1-(prop-2-enoyl)pyrrolidin-2-yl]methoxy]pyridin-4-yl)-1H,5H,6H,7H-pyrrolo[3,2-c]pyridin-4-one (compound 138a)

### 20.1. Synthesis of 3-[(3-chloro-2-methoxyphenyl)amino]-2-(3-[[(2R)-1-(prop-2-enoyl)pyrrolidin-2-yl]methoxy]pyridin-4-yl)-1H,5H,6H,7H-pyrrolo[3,2-c]pyridin-4-one

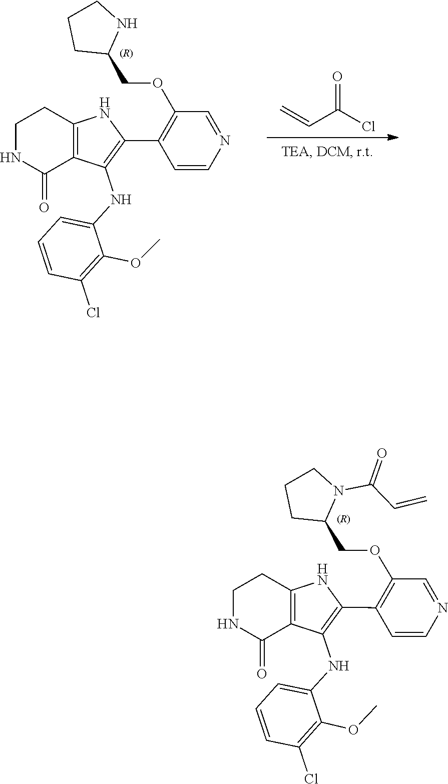

A solution of 3-[(3-chloro-2-methoxyphenyl)amino]-2-[3-[(2R)-pyrrolidin-2-ylmethoxy]pyridin-4-yl]-1H,5H,6H,7H-pyrrolo[3,2-c]pyridin-4-one (50 mg, 0.1 mmol, 1.00 equiv) and TEA (21 mg, 0.2 mmol, 2.00 equiv) in DCM (1.00 mL) was added acryloyl chloride (9 mg, 0.1 mmol, 1.00 equiv) at 0° C., stirred for 1 h at room temperature under nitrogen atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: Water (10 MMOL/L NH4HCO3), Mobile Phase B: ACN; Flow rate: 60 mL/min; Gradient: 25% B to 45% B in 8 min; Wave Length: 254 nm; RT1(min): 7.5; Injection Volumn: 1 ml; Number Of Runs:2;) to afford 3-[(3-chloro-2-methoxyphenyl)amino]-2-(3-[[(2R)-1-(prop-2-enoyl)pyrrolidin-2-yl]methoxy]pyridin-4-yl)-1H,5H,6H,7H-pyrrolo[3,2-c]pyridin-4-one (4.3 mg, 7.71%) as a yellow solid.

LC-MS: (M+H)+ found: 522.1.

¹H NMR (400 MHz, DMSO-d6) δ 11.52 (s, 1H), 8.40 (s, 1H), 7.97 (d, J=5.0 Hz, 1H), 7.42-7.33 (m, 2H), 7.13-7.04 (m, 1H), 6.69-6.53 (m, 3H), 6.21 (dd, J=16.7, 2.3 Hz, 1H), 6.15-5.98 (m, 1H), 5.73 (dd, J=10.3, 2.3 Hz, 1H), 4.64 (s, 1H), 4.34 (dd, J=9.8, 7.1 Hz, 1H), 4.11 (ddd, J=30.2, 9.8, 5.1 Hz, 1H), 3.84 (d, J=17.9 Hz, 3H), 3.62 (dd, J=7.5, 4.5 Hz, 2H), 2.96 (t, J=6.8 Hz, 2H), 1.99 (dq, J=13.7, 7.3 Hz, 2H), 1.95-1.83 (m, 4H).

### Example 21. 3-[(3-fluoro-2-methoxyphenyl)amino]-2-(3-[5H,6H,7H-pyrrolo[1,2-a]imidazol-7-ylmethoxy]pyridin-4-yl)-1H,5H,6H,7H-pyrrolo[3,2-c]pyridin-4-one (compound 124)

### 21.1. Synthesis of 3-[5H,6H,7H-pyrrolo[1,2-a]imidazol-7-ylmethoxy]pyridine-4-carbonitrile

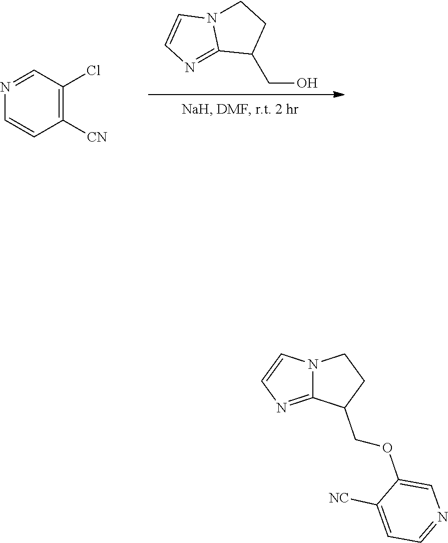 [SMILES: N#Cc1ccncc1Cl ; OCC1CCn2ccnc21 ; N#Cc1ccncc1OCC1CCn2ccnc21]

To a mixture of 5H,6H,7H-pyrrolo[1,2-a]imidazol-7-ylmethanol (138 mg, 1.0 mmol, 1.00 equiv) in DMF (2 mL) was added NaH (48 mg, 2.0 mmol, 2.00 equiv) at 0° C. The mixture was stirred for 0.5 h at room temperature and 3-chloropyridine-4-carbonitrile (139 mg, 1.0 mmol, 1.00 equiv) was added. The result mixture was stirred for 2h at room temperature. The reaction was quenched with Water at room temperature. The resulting mixture was extracted with EtOAc (3×10 mL). The combined organic layers were washed with brine (1×10 mL), dried over anhydrous Na2SO4. After filtration, the filtrate was concentrated under reduced pressure. The residue was purified by Prep-TLC (CH2Cl2/MeOH 15:1) to afford 3-[5H,6H,7H-pyrrolo[1,2-a]imidazol-7-ylmethoxy]pyridine-4-carbonitrile (144 mg, 59.74%) as an off-white solid.

LC-MS: (M+H)+ found: 181.6.

### 21.2. Synthesis of -[5H,6H,7H-pyrrolo[1,2-a]imidazol-7-ylmethoxy]pyridin-4-yl)methanamine

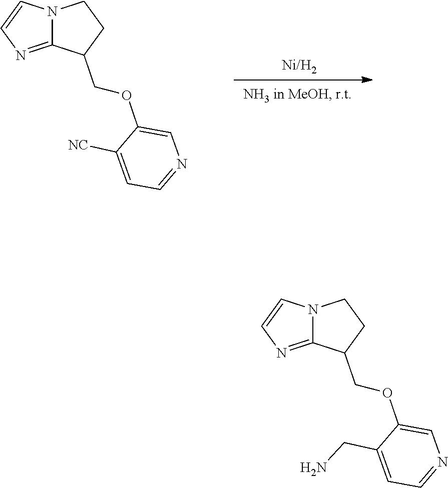 [SMILES: N#Cc1ccncc1OCC1CCn2ccnc21 ; NCc1ccncc1OCC1CCn2ccnc21]

A solution of 3-[5H,6H,7H-pyrrolo[1,2-a]imidazol-7-ylmethoxy]pyridine-4-carbonitrile (140 mg, 0.58 mmol, 1.00 equiv) and Raney Ni (199 mg, 2.33 mmol, 4.00 equiv) in NH3 (5 mL, 7M in MeOH) and MeOH (5 mL) was stirred for 2 h at room temperature under hydrogen atmosphere. The resulting mixture was filtered, the filter cake was washed with MeOH (1×10 mL). The filtrate was concentrated under reduced pressure. The residue was purified by Prep-TLC (CH2Cl2/MeOH 1:1) to afford 1-(3-[5H,6H,7H-pyrrolo[1,2-a]imidazol-7-ylmethoxy]pyridin-4-yl)methanamine (80 mg, 56.20%) as a yellow oil.

LC-MS: (M+H)+ found 245.1.

### 21.3. Synthesis of tert-butyl 3-[(3-fluoro-2-methoxyphenyl)carbamothioyl]-2-oxo-4-[[(3-[5H,6H,7H-pyrrolo[1,2-a]imidazol-7-ylmethoxy]pyridin-4-yl)methyl]amino]-5,6-dihydropyridine-1-carboxylate

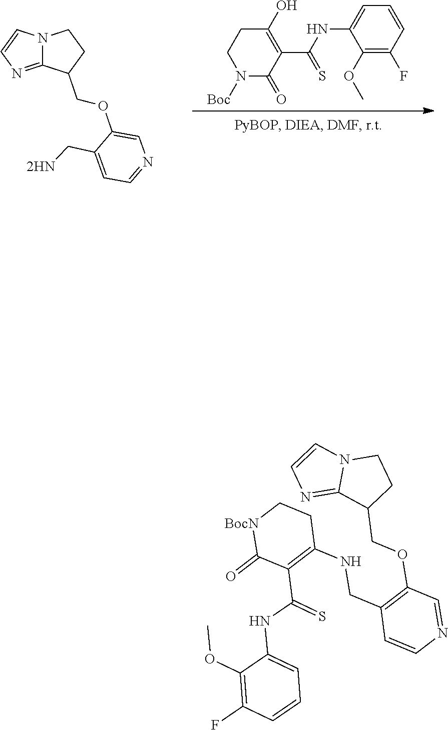

A solution of 1-(3-[5H,6H,7H-pyrrolo[1,2-a]imidazol-7-ylmethoxy]pyridin-4-yl)methanamine (80 mg, 0.32 mmol, 1.00 equiv) and tert-butyl 3-[(3-fluoro-2-methoxyphenyl)carbamothioyl]-4-hydroxy-2-oxo-5,6-dihydropyridine-1-carboxylate (129 mg, 0.32 mmol, 1.00 equiv), PyBOP (221 mg, 0.42 mmol, 1.30 equiv), DIEA (126 mg, 0.98 mmol, 3.00 equiv) in DMF (2 mL) was stirred for 2h at room temperature under nitrogen atmosphere. The resulting mixture was extracted with EtOAc (3×10 mL). The combined organic layers were washed with brine (1×10 mL), dried over anhydrous Na2SO4. After filtration, the filtrate was concentrated under reduced pressure. The residue was purified by Prep-TLC (PE/EtOAc 1:1) to afford tert-butyl 3-[(3-fluoro-2-methoxyphenyl)carbamothioyl]-2-oxo-4-[[(3-[5H,6H,7H-pyrrolo[1,2-a]imidazol-7-ylmethoxy]pyridin-4-yl)methyl]amino]-5,6-dihydropyridine-1-carboxylate (65 mg, 31.88%) as a yellow oil.

LC-MS: (M-56)+ found 623.2.

### 21.4. Synthesis of 3-[(3-fluoro-2-methoxyphenyl)amino]-2-(3-[5H,6H,7H-pyrrolo[1,2-a]imidazol-7-ylmethoxy]pyridin-4-yl)-1H,5H,6H,7H-pyrrolo[3,2-c]pyridin-4-one

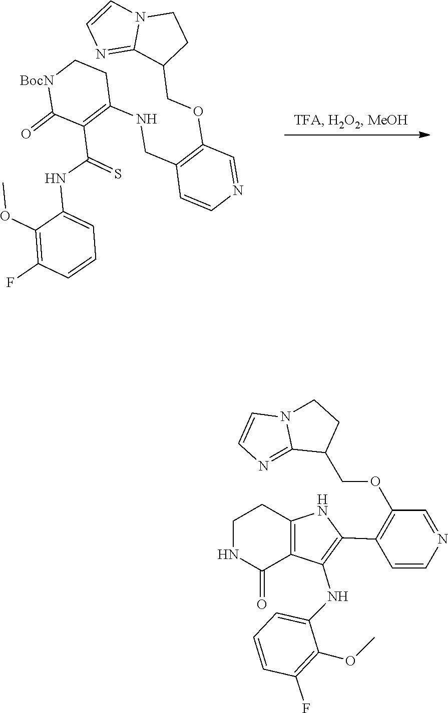 [SMILES: COc1c(F)cccc1NC(=S)C1=C(NCc2ccncc2OCC2CCn3ccnc32)CCN(C(=O)OC(C)(C)C)C1=O ; COc1c(F)cccc1Nc1c(-c2ccncc2OCC2CCn3ccnc32)[nH]c2c1C(=O)NCC2]

A solution of tert-butyl 3-[(3-fluoro-2-methoxyphenyl)carbamothioyl]-2-oxo-4-[[(3-[5H,6H,7H-pyrrolo[1,2-a]imidazol-7-ylmethoxy]pyridin-4-yl)methyl]amino]-5,6-dihydropyridine-1-carboxylate (65 mg, 0.10 mmol, 1.00 equiv), TFA (0.03 mL, 0.41 mmol, 4.00 equiv) and H₂O₂ (30%) (7 mg, 0.20 mmol, 2.00 equiv) in MeOH (1 mL) was stirred for 1 h at 60° C. under nitrogen atmosphere. The reaction was quenched by the addition of sat. Na2SO3 (aq.) (1 mL) at room temperature. The resulting mixture was concentrated under reduced pressure. The residue was purified by reverse phase flash with the following conditions (MeCN/H₂O-40%) to afford 3-[(3-fluoro-2-methoxyphenyl)amino]-2-(3-[5H,6H,7H-pyrrolo[1,2-a]imidazol-7-ylmethoxy]pyridin-4-yl)-1H,5H,6H,7H-pyrrolo[3,2-c]pyridin-4-one (2.5 mg, 4.90%) as an off-white solid.

LC-MS: (M+H)+ found 489.1

¹H NMR (300 MHz, DMSO-d6) δ 13.49 (s, 1H), 8.37 (m, 2H), 7.96 (d, J=5.0 Hz, 1H), 7.61 (s, 1H), 7.34 (d, J=5.1 Hz, 1H), 7.23 (d, J=1.2 Hz, 1H), 7.17-7.07 (m, 2H), 6.66 (td, J=8.2, 6.0 Hz, 1H), 6.51 (ddd, J=10.0, 8.4, 1.4 Hz, 1H), 6.05 (d, J=8.2 Hz, 1H), 4.68 (dd, J=8.9, 5.1 Hz, 1H), 4.13 (dt, J=9.1, 5.3 Hz, 2H), 4.05 (dd, J=12.0, 8.4 Hz, 3H), 3.94 (s, 2H), 3.75 (s, 2H), 3.12-3.00 (m, 3H).

### Example 22. 3-[(3-fluoro-2-methoxyphenyl)amino]-2-(3-[imidazo[2,1-b][1,3]thiazol-3-ylmethoxy]pyridin-4-yl)-1H,5H,6H,7H-pyrrolo[3,2-c]pyridin-4-one (compound 130)

### 22.1. Synthesis of 3-[imidazo[2,1-b][1,3]thiazol-3-ylmethoxy]pyridine-4-carbonitrile

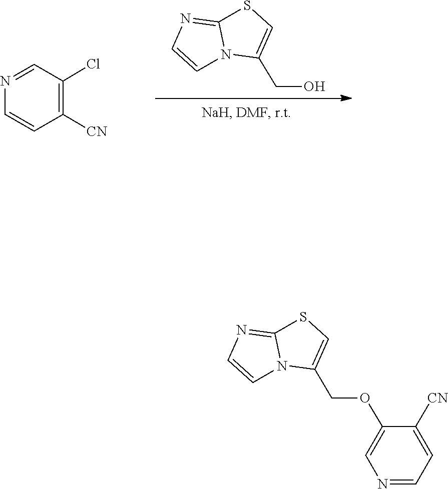 [SMILES: N#Cc1ccncc1Cl ; OCc1csc2nccn12 ; N#Cc1ccncc1OCc1csc2nccn12]

To a mixture of imidazo[2,1-b][1,3]thiazol-3-ylmethanol (155.80 mg, 1.010 mmol, 1.00 equiv) in DMF (2.00 mL) was added NaH (80.83 mg, 2.020 mmol, 2.00 equiv, 60%) and stirred for 0.5 h at 0 degree C. To the mixture was added 3-chloropyridine-4-carbonitrile (140.00 mg, 1.010 mmol, 1.00 equiv) and stirred for 2h at room temperature under nitrogen atmosphere. The reaction was quenched with Water at room temperature. The resulting mixture was extracted with EtOAc (3×25 mL). The combined organic layers were washed with brine (1×10 mL), dried over anhydrous Na2SO4. After filtration, the filtrate was concentrated under reduced pressure. The residue was purified by Prep-TLC (CH2Cl2/MeOH 12:1) to afford 3-[imidazo[2,1-b][1,3]thiazol-3-ylmethoxy]pyridine-4-carbonitrile (215 mg, 83.02%) as a light yellow solid.

LC-MS: (M+H)+ found: 257.15.

### 22.2. Synthesis of 1-(3-[4H,5H,6H-pyrrolo[1,2-b]pyrazol-3-ylmethoxy]pyridin-4-yl)methanamine

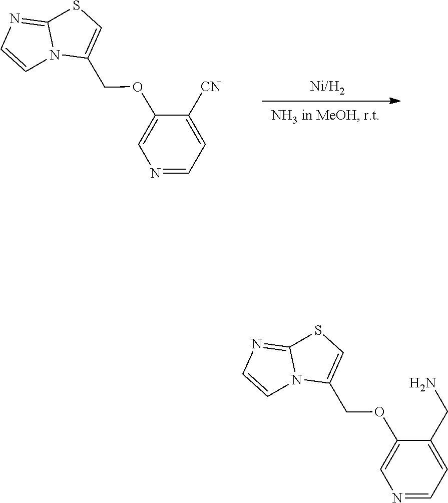 [SMILES: N#Cc1ccncc1OCc1csc2nccn12 ; NCc1ccncc1OCc1csc2nccn12]

To a solution of 3-[imidazo[2,1-b][1,3]thiazol-3-ylmethoxy]pyridine-4-carbonitrile (184.00 mg, 0.718 mmol, 1.00 equiv) in Ammonia, 7.0 M Solution in MeOH (5.00 mL) was added Raney-Ni (123.02 mg, 1.436 mmol, 2.00 equiv) under nitrogen atmosphere. The mixture was hydrogenated at room temperature for 4 h under hydrogen atmosphere using a hydrogen balloon, filtered through a Celite pad and concentrated under reduced pressure. The resulting mixture was diluted with CH2Cl2 (2 mL). The residue was purified by Prep-TLC (CH2Cl2/MeOH 10:1) to afford 1-(3-[4H,5H,6H-pyrrolo[1,2-b]pyrazol-3-ylmethoxy]pyridin-4-yl)methanamine (134 mg, 48.25%) as a light yellow oil.

LC-MS: (M+H)+ found 261.

### 22.3. Synthesis of tert-butyl 3-[(3-fluoro-2-methoxyphenyl)carbamothioyl]-2-oxo-4-[[(3-[4H,5H,6H-pyrrolo[1,2-b]pyrazol-3-ylmethoxy]pyridin-4-yl)methyl]amino]-5,6-dihydropyridine-1-carboxylate

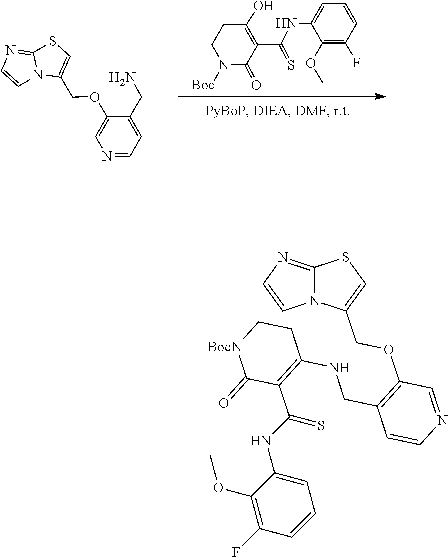 [SMILES: NCc1ccncc1OCc1csc2nccn12 ; COc1c(F)cccc1NC(=S)C1=C(O)CCN(C(=O)OC(C)(C)C)C1=O ; COc1c(F)cccc1NC(=S)C1=C(NCc2ccncc2OCc2csc3nccn23)CCN(C(=O)OC(C)(C)C)C1=O]

To a stirred mixture of tert-butyl 3-[(3-fluoro-2-methoxyphenyl)carbamothioyl]-4-hydroxy-2-oxo-5,6-dihydropyridine-1-carboxylate (180.00 mg, 0.454 mmol, 1.00 equiv), 1-(3-[4H,5H,6H-pyrrolo[1,2-b]pyrazol-3-ylmethoxy]pyridin-4-yl)methanamine (122.02 mg, 0.499 mmol, 1.10 equiv) and PyBOP (354.43 mg, 0.681 mmol, 1.50 equiv) in DMF (5.00 mL) was added DIEA (117.37 mg, 0.908 mmol, 2.00 equiv) dropwise at room temperature under nitrogen atmosphere. The mixture was stirred for 2 h at 60 degrees C. The resulting mixture was extracted with CH2Cl2 (3×30 mL). The combined organic layers were washed with brine (2×20 mL), dried over anhydrous Na2SO4. After filtration, the filtrate was concentrated under reduced pressure. The residue was purified by Prep-TLC (CH2Cl2/MeOH 10:1) to afford tert-butyl 3-[(3-fluoro-2-methoxyphenyl)carbamothioyl]-2-oxo-4-[[(3-[4H,5H,6H-pyrrolo[1,2-b]pyrazol-3-ylmethoxy]pyridin-4-yl)methyl]amino]-5,6-dihydropyridine-1-carboxylate (300 mg, 96.76%) as a yellow solid.

LC-MS: (M+1)+ found 639.1.

### 22.4. Synthesis of 3-[(3-fluoro-2-methoxyphenyl)amino]-2-(3-[imidazo[2,1-b][1,3]thiazol-3-ylmethoxy]pyridin-4-yl)-1H,5H,6H,7H-pyrrolo[3,2-c]pyridin-4-one

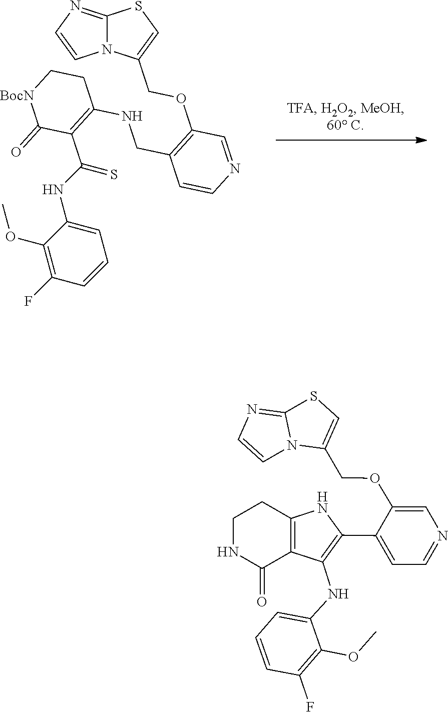 [SMILES: COc1c(F)cccc1NC(=S)C1=C(NCc2ccncc2OCc2csc3nccn23)CCN(C(=O)OC(C)(C)C)C1=O ; COc1c(F)cccc1Nc1c(-c2ccncc2OCc2csc3nccn23)[nH]c2c1C(=O)NCC2]

A solution of tert-butyl 3-[(3-fluoro-2-methoxyphenyl)carbamothioyl]-4-[[(3-[imidazo[2,1-b][1,3]thiazol-3-ylmethoxy]pyridin-4-yl)methyl]amino]-2-oxo-5,6-dihydropyridine-1-carboxylate (130.00 mg, 0.204 mmol, 1.00 equiv) in MeOH (5.00 mL) was treated with H₂O₂ (30%)(13.85 mg, 0.408 mmol, 2.00 equiv) for 2 min at room temperature under nitrogen atmosphere followed by the addition of TFA (92.83 mg, 0.816 mmol, 4.00 equiv) dropwise at room temperature. The resulting mixture was stirred for 3 h at 60 degrees C. under nitrogen atmosphere. The resulting mixture was extracted with CH2Cl2 (3×30 mL). The combined organic layers were washed with brine (2×20 mL), dried over anhydrous Na2SO4. After filtration, the filtrate was concentrated under reduced pressure. The crude product (30 mg) was purified by Prep-HPLC with the following conditions (Column: XSelect CSH Fluoro Phenyl, 30*150 mm, 5 μm; Mobile Phase A: Water (10 MMOL/L NH4HCO3), Mobile Phase B: MeOH; Flow rate: 60 mL/min; Gradient: 42% B to 55% B in 10 min; Wave Length: 254 nm; RT1(min): 9.08;) to afford 3-[(3-fluoro-2-methoxyphenyl)amino]-2-(3-[imidazo[2,1-b][1,3]thiazol-3-ylmethoxy]pyridin-4-yl)-1H,5H,6H,7H-pyrrolo[3,2-c]pyridin-4-one (3.5 mg, 3.41%) as a yellow solid.

LC-MS: (M+H)+ found 505.05.

¹H NMR (400 MHz, DMSO-d6) δ 11.18 (s, 1H), 8.63 (s, 1H), 8.03 (d, J=5.1 Hz, 1H), 7.73 (d, J=1.5 Hz, 1H), 7.45-7.35 (m, 2H), 7.28 (d, J=5.0 Hz, 1H), 7.22 (t, J=1.3 Hz, 1H), 7.16-7.06 (m, 1H), 6.54-6.39 (m, 2H), 5.71 (m, J=7.9, 1.5 Hz, 1H), 5.56 (s, 2H), 3.85 (d, J=0.6 Hz, 3H), 3.41 (dd, J=6.9, 2.5 Hz, 2H), 2.83 (t, J=6.8 Hz, 2H).

### Example 23. 3-[(3-fluoro-2-methoxyphenyl)amino]-2-(3-[5H,6H,7H-pyrrolo[2,1-c][1,2,4]triazol-3-ylmethoxy]pyridin-4-yl)-1H,5H,6H,7H-pyrrolo[3,2-c]pyridin-4-one (compound 131)

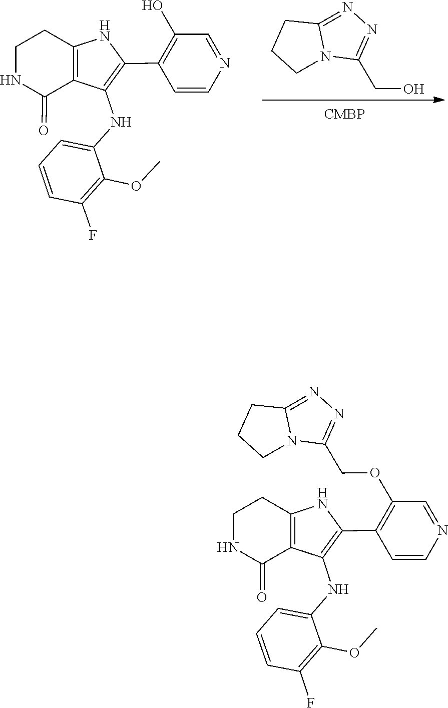 [SMILES: COc1c(F)cccc1Nc1c(-c2ccncc2O)[nH]c2c1C(=O)NCC2 ; OCc1nnc2n1CCC2 ; COc1c(F)cccc1Nc1c(-c2ccncc2OCc2nnc3n2CCC3)[nH]c2c1C(=O)NCC2]

To a stirred mixture of 3-[(3-fluoro-2-methoxyphenyl)amino]-2-(3-hydroxypyridin-4-yl)-1H,5H,6H,7H-pyrrolo[3,2-c]pyridin-4-one (200.00 mg, 0.5 mmol, 1.00 equiv) and 5H,6H,7H-pyrrolo[2,1-c][1,2,4]triazol-3-ylmethanol (113.33 mg, 0.8 mmol, 1.50 equiv) in Toluene (2.00 mL) was added CMBP (392.54 mg, 1.6 mmol, 3.00 equiv) at room temperature under argon atmosphere. The resulting mixture was stirred for 2 h at 90 degrees C. under argon atmosphere. The resulting mixture was concentrated under vacuum. The resulting mixture was concentrated under reduced pressure and the crude product (200 mg) was purified by Prep-HPLC with the following conditions (Column: XBridge Shield RP18 OBD Column, 30*150 mm, 5 μm; Mobile Phase A: Water (10 MMOL/L NH4HCO3), Mobile Phase B: ACN; Flow rate: 60 mL/min; Gradient: 18% B to 30% B in 8 min; Wave Length: 220 nm; RT1(min): 7.5;) to afford 3-[(3-fluoro-2-methoxyphenyl)amino]-2-(3-[5H,6H,7H-pyrrolo[2,1-c][1,2,4]triazol-3-ylmethoxy]pyridin-4-yl)-1H,5H,6H,7H-pyrrolo[3,2-c]pyridin-4-one (5.8 mg, 2.18%) as a white solid.

LC-MS: (M+H)+ found 490.35.

¹H NMR (300 MHz, DMSO-d6) δ 11.71 (s, 1H), 8.55 (s, 1H), 8.07 (d, J=5.0 Hz, 1H), 7.44 (s, 1H), 7.32 (d, J=5.0 Hz, 1H), 7.13 (s, 1H), 6.61 (m, 1H), 6.47 (m, 1H), 5.96 (m, 1H), 5.50 (s, 2H), 3.92-3.82 (m, 5H), 3.42 (m, 2H), 2.84 (m, 4H), 2.61 (q, J=7.3 Hz, 2H).

### Example 24. 3-[(3-chloro-2-methoxyphenyl)amino]-2-(3-[[(2R)-1-propanoylpyrrolidin-2-yl]methoxy]pyridin-4-yl)-1H,5H,6H,7H-pyrrolo[3,2-c]pyridin-4-one (compound 470)

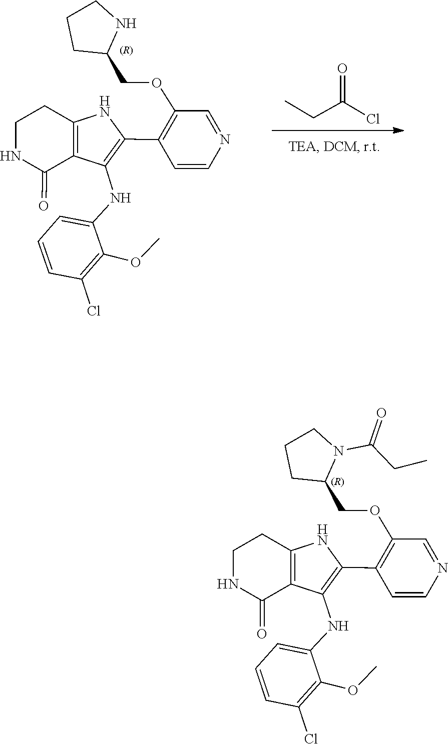

To a solution of 3-[(3-chloro-2-methoxyphenyl)amino]-2-[3-[(2R)-pyrrolidin-2-ylmethoxy]pyridin-4-yl]-1H,5H,6H,7H-pyrrolo[3,2-c]pyridin-4-one (50 mg, 0.1 mmol, 1.00 equiv) and TEA (21 mg, 0.2 mmol, 2.00 equiv) in DCM (1.00 mL) was added propanoyl chloride (9 mg, 0.1 mmol, 1.00 equiv) at ° C., stirred for 1 h at room temperature under nitrogen atmosphere. The resulting mixture was concentrated under reduced pressure. The crude product was purified by Prep-HPLC with the following conditions (Column: XBridge Prep C18 OBD Column, 19*150 mm, 5 μm; Mobile Phase A: Water (10MMOL/L NH4HCO3), Mobile Phase B: ACN; Flow rate: 60 mL/min; Gradient: 41% B to 45% B in 8 min; Wave Length: 254 nm; RT1(min): 6;) to afford 3-[(3-chloro-2-methoxyphenyl)amino]-2-(3-[[(2R)-1-propanoylpyrrolidin-2-yl]methoxy]pyridin-4-yl)-1H,5H,6H,7H-pyrrolo[3,2-c]pyridin-4-one (12.9 mg, 23.04%) as a yellow solid.

LC-MS: (M+H)+ found: 524.1.

¹H NMR (400 MHz, DMSO-d6) δ 11.53 (s, 1H), 8.39 (s, 1H), 7.97 (d, J=5.0 Hz, 1H), 7.45 (s, 1H), 7.33 (d, J=5.0 Hz, 1H), 7.12 (d, J=2.6 Hz, 1H), 6.71-6.61 (m, 2H), 6.16-6.07 (m, 1H), 4.58-4.52 (m, 1H), 4.29 (dd, J=9.8, 7.2 Hz, 1H), 4.11 (dd, J=9.8, 4.1 Hz, 1H), 3.87 (s, 3H), 3.57-3.44 (m, 4H), 2.96 (td, J=6.6, 2.1 Hz, 2H), 2.38-2.22 (m, 2H), 1.92 (ddd, J=29.2, 14.0, 8.4 Hz, 4H), 0.98 (t, J=7.3 Hz, 3H).

### Example 25. 3-[(3-fluoro-2-methoxyphenyl)amino]-2-[3-(pyridin-2-ylmethoxy)pyridin-4-yl]-1H,5H,6H,7H-pyrrolo[3,2-c]pyridin-4-one (compound 472)

### 25.1. Synthesis of 1-fluoro-3-isothiocyanato-2-methoxybenzene

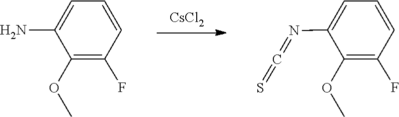

To a stirred mixture of 3-fluoro-2-methoxyaniline (5.00 g, 35.425 mmol, 1.00 equiv) and sat. NaHCO₃ (50 mL) in DCM (50.00 mL) was added thiophosgene (8.15 g, 70.849 mmol, 1.00 equiv) dropwise at 0 degrees C. under N₂ atmosphere. The resulting mixture was stirred for 2 h at 0 degrees C. TLC (EtOAc:Hexane) showed complete conversion. The DCM layer was separated and washed with sat. NaHCO₃, brine, filtered through a hydrophobic filter and concentrated to give 1-fluoro-3-isothiocyanato-2-methoxybenzene (12 g, 92.45%) as a yellow oil.

### 25.2. Synthesis of tert-butyl 3-[(3-fluoro-2-methoxyphenyl)carbamothioyl]-4-hydroxy-2-oxo-5,6-dihydropyridine-1-carboxylate

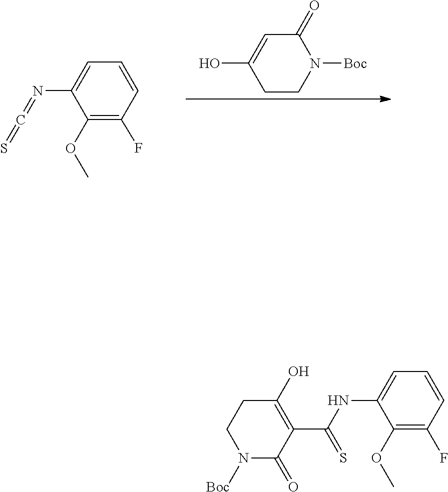 [SMILES: COc1c(F)cccc1N=C=S ; CC(C)(C)OC(=O)N1CCC(O)=CC1=O ; COc1c(F)cccc1NC(=S)C1=C(O)CCN(C(=O)OC(C)(C)C)C1=O]

To a stirred mixture of 1-fluoro-3-isothiocyanato-2-methoxybenzene (12.00 g, 65.502 mmol, 1.00 equiv) and tert-butyl 4-hydroxy-2-oxo-5,6-dihydropyridine-1-carboxylate (13.97 g, 65.502 mmol, 1.00 equiv) in MeCN (100.00 mL) was added DBU (14.96 g, 98.253 mmol, 1.50 equiv) dropwise at 0 degrees C. The resulting mixture was stirred for 2 h at 0 degrees C. The reaction was quenched with water at 0 degrees C. The mixture was acidified to pH 7 with con. HCl. The precipitated solids were collected by filtration, washed with water and concentrated under reduced pressure. This resulted in tert-butyl 3-[(3-fluoro-2-methoxyphenyl)carbamothioyl]-4-hydroxy-2-oxo-5,6-dihydropyridine-1-carboxylate (19.5 g, 75.10%) as a light yellow solid.

### 25.3. Synthesis of 3-(benzyloxy)pyridine-4-carbonitrile

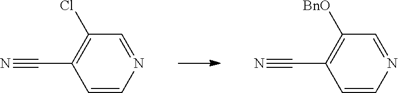 [SMILES: N#Cc1ccncc1Cl ; N#Cc1ccncc1OCc1ccccc1]

A solution of benzyl alcohol (9.37 g, 86.611 mmol, 1.20 equiv) in DMF (70.00 mL) was treated with NaH (3.75 g, 93.829 mmol, 1.30 equiv, 60%) for 5 min at 0 degrees C. under nitrogen atmosphere at room temperature. The resulting mixture was stirred for 30 min at room temperature under nitrogen atmosphere. To the above mixture was added 3-chloropyridine-4-carbonitrile (10.00 g, 72.176 mmol, 1.00 equiv) in portions over 5 min at room temperature. The resulting mixture was stirred for additional overnight at room temperature. The reaction was quenched by the addition of saturated NH₄Cl aqueous solution (10 mL) at room temperature. The resulting mixture was extracted with EtOAc (3×200 mL). The combined organic layers were washed with brine (2×200 mL), dried over anhydrous Na2SO4. After filtration, the filtrate was concentrated under reduced pressure. This resulted in 3-(benzyloxy)pyridine-4-carbonitrile (15 g, 84.03%) as a yellow solid.

LC-MS: M+H found: 211.1.

### 25.4. Synthesis of 1-[3-(benzyloxy)pyridin-4-yl]methanamine

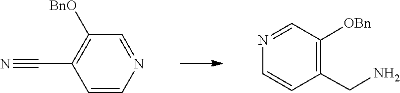 [SMILES: N#Cc1ccncc1OCc1ccccc1 ; NCc1ccncc1OCc1ccccc1]

To a solution of 3-(benzyloxy)pyridine-4-carbonitrile (15.00 g, 71.348 mmol, 1.00 equiv) in Ammonia (7.0 M Solution In Ethanol, 150.00 mL) was added Raney nickel (9.17 g, 107.033 mmol, 1.50 equiv) under nitrogen atmosphere in a 250 mL round-bottom flask. The mixture was hydrogenated at room temperature for 2 h under hydrogen atmosphere using a hydrogen balloon, filtered through a Celite pad and concentrated under reduced pressure. The residue was purified by reverse flash chromatography with the following conditions: column, C18 silica gel; mobile phase, ACN in water, 10% to 30% gradient in 30 min; detector, UV 254 nm. This resulted in 1-[3-(benzyloxy)pyridin-4-yl]methanamine (7 g, 44.64%) as a colorless oil.

LC-MS: M+H found: 215.20.

### 25.5. Synthesis of tert-butyl 4-([[3-(benzyloxy)pyridin-4-yl]methyl]amino)-3-[(3-fluoro-2-methoxyphenyl)carbamothioyl]-2-oxo-5,6-dihydropyridine-1-carboxylate

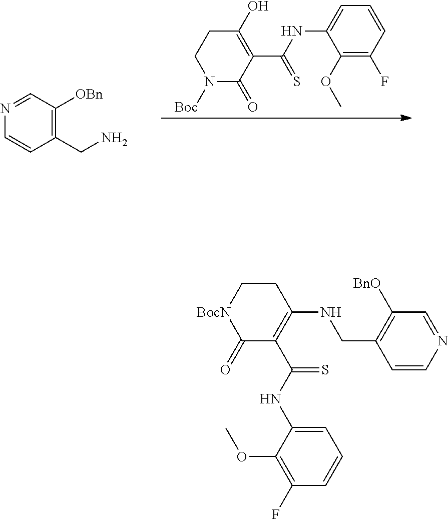 [SMILES: NCc1ccncc1OCc1ccccc1 ; COc1c(F)cccc1NC(=S)C1=C(O)CCN(C(=O)OC(C)(C)C)C1=O ; COc1c(F)cccc1NC(=S)C1=C(NCc2ccncc2OCc2ccccc2)CCN(C(=O)OC(C)(C)C)C1=O]

To a stirred mixture of tert-butyl 3-[(3-fluoro-2-methoxyphenyl)carbamothioyl]-4-hydroxy-2-oxo-5,6-dihydropyridine-1-carboxylate (8.20 g, 20.685 mmol, 1.00 equiv) and PyBOP (11.84 g, 22.754 mmol, 1.10 equiv) in DMF (80.00 mL) were added DIEA (5.35 g, 41.370 mmol, 2.00 equiv) and 1-[3-(benzyloxy)pyridin-4-yl]methanamine (4.88 g, 22.753 mmol, 1.10 equiv) at room temperature under nitrogen atmosphere. The resulting mixture was stirred for overnight at room temperature. The reaction was monitored by LCMS. Desired product could be detected by LCMS. The resulting mixture was extracted with EtOAc (3×200 mL). The combined organic layers were washed with xylene (3×500 mL), dried over anhydrous Na2SO4. After filtration, the filtrate was concentrated under reduced pressure. The residue was purified by silica gel column chromatography, eluted with PE/EtOAc (10:1) to afford tert-butyl 4-([[3-(benzyloxy)pyridin-4-yl]methyl]amino)-3-[(3-fluoro-2-methoxyphenyl)carbamothioyl]-2-oxo-5,6-dihydropyridine-1-carboxylate (4.0 g, 28.39%) as a orange solid.

LC-MS: M+H found: 593.15

### 25.6. Synthesis of 2-[3-(benzyloxy)pyridin-4-yl]-3-[(3-fluoro-2-methoxyphenyl)amino]-1H,5H,6H,7H-pyrrolo[3,2-c]pyridin-4-one

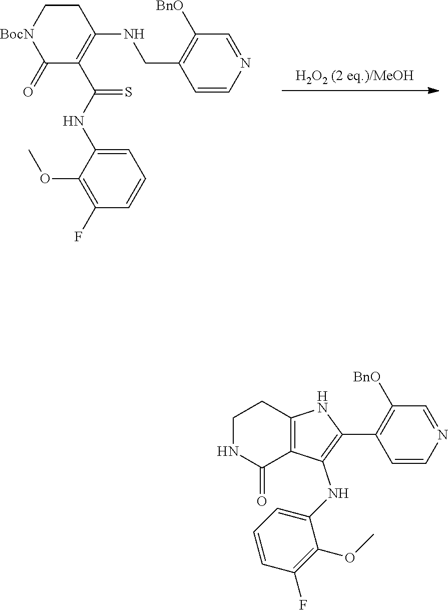 [SMILES: COc1c(F)cccc1NC(=S)C1=C(NCc2ccncc2OCc2ccccc2)CCN(C(=O)OC(C)(C)C)C1=O ; COc1c(F)cccc1Nc1c(-c2ccncc2OCc2ccccc2)[nH]c2c1C(=O)NCC2]

To a stirred mixture of tert-butyl 4-([[3-(benzyloxy)pyridin-4-yl]methyl]amino)-3-[(3-fluoro-2-methoxyphenyl)carbamothioyl]-2-oxo-5,6-dihydropyridine-1-carboxylate (3.80 g, 6.411 mmol, 1.00 equiv) and H₂O₂ (2.18 g, 19.227 mmol, 3.00 equiv, 30%) in MeOH (40.00 mL) was added TFA (1.10 g, 9.617 mmol, 1.50 equiv) at room temperature under nitrogen atmosphere. The resulting mixture was stirred for 1 h at 80 degrees C. The reaction was monitored by LCMS. Desired product could be detected by LCMS. The reaction was quenched with sat. NaHSO3 (aq.) at room temperature. The resulting mixture was concentrated under reduced pressure. The residue was purified by reverse flash chromatography with the following conditions: column, C18 silica gel; mobile phase, ACN in water, 10% to 50% gradient in 10 min; detector, UV 254 nm. This resulted in 2-[3-(benzyloxy)pyridin-4-yl]-3-[(3-fluoro-2-methoxyphenyl)amino]-1H,5H,6H,7H-pyrrolo[3,2-c]pyridin-4-one (1.3 g, 32.73%) as a orange oil.

LC-MS: M+H found: 459.00.

### 25.7. Synthesis of 3-[(3-fluoro-2-methoxyphenyl)amino]-2-(3-hydroxypyridin-4-yl)-1H,5H,6H,7H-pyrrolo[3,2-c]pyridin-4-one

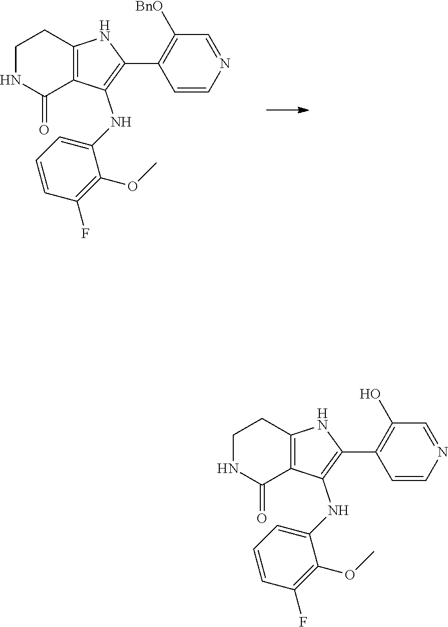 [SMILES: COc1c(F)cccc1Nc1c(-c2ccncc2OCc2ccccc2)[nH]c2c1C(=O)NCC2 ; COc1c(F)cccc1Nc1c(-c2ccncc2O)[nH]c2c1C(=O)NCC2]

To a stirred mixture of 2-[3-(benzyloxy)pyridin-4-yl]-3-[(3-fluoro-2-methoxyphenyl)amino]-1H,5H,6H,7H-pyrrolo[3,2-c]pyridin-4-one (600.00 mg, 1.309 mmol, 1.00 equiv) in MeOH/AcOH (6.00 mL/6.00 mL) was added Pd/C (278.53 mg, 2.617 mmol, 2.00 equiv) at room temperature under nitrogen atmosphere. The resulting mixture was stirred for overnight at 50 degrees C. under hydrogen atmosphere. The reaction was monitored by LCMS. Desired product could be detected by LCMS. The resulting mixture was filtered, the filter cake was washed with MeOH (3×10 mL). The filtrate was concentrated under reduced pressure. The residue was purified by Prep-TLC (CH2Cl2/MeOH 10:1) to afford 3-[(3-fluoro-2-methoxyphenyl)amino]-2-(3-hydroxypyridin-4-yl)-1H,5H,6H,7H-pyrrolo[3,2-c]pyridin-4-one (200 mg, 36.80%) as a yellow solid.

LC-MS: M+H found: 368.95.

### 25.8. Synthesis of 3-[(3-fluoro-2-methoxyphenyl)amino]-2-[3-(pyridin-2-ylmethoxy)pyridin-4-yl]-1H,5H,6H,7H-pyrrolo[3,2-c]pyridin-4-one

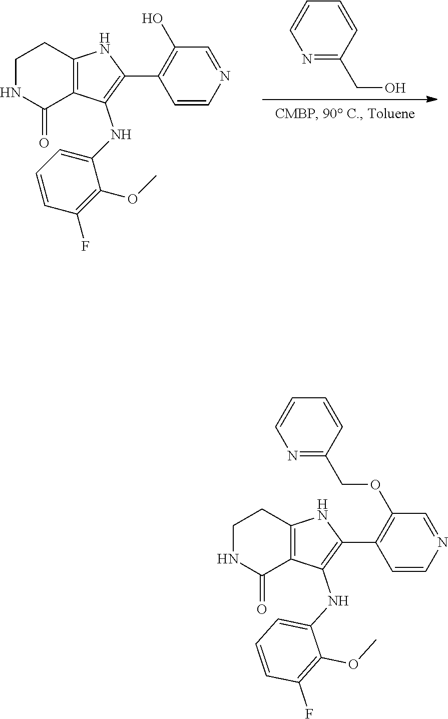 [SMILES: COc1c(F)cccc1Nc1c(-c2ccncc2O)[nH]c2c1C(=O)NCC2 ; OCc1ccccn1 ; COc1c(F)cccc1Nc1c(-c2ccncc2OCc2ccccn2)[nH]c2c1C(=O)NCC2]

A solution of 3-[(3-fluoro-2-methoxyphenyl)amino]-2-(3-hydroxypyridin-4-yl)-1H,5H,6H,7H-pyrrolo[3,2-c]pyridin-4-one (100.00 mg, 0.271 mmol, 1.00 equiv) and 2-pyridinemethanol (59.25 mg, 0.543 mmol, 2.00 equiv) in Toluene (1.00 mL) was treated with 2-(tributyl-lambda 5-phosphanylidene)acetonitrile (131.04 mg, 0.543 mmol, 2.00 equiv), stirred for overnight at 90 degrees C. under nitrogen atmosphere. The resulting mixture was concentrated under reduced pressure. The resulting mixture was diluted with 2 ml DMF. The crude product (100 mg) was purified by Prep-HPLC with the following conditions (Column: Xselect CSH F-Phenyl OBD column, 19*250 mm, 5 μm; Mobile Phase A: Water (0.05% FA), Mobile Phase B: ACN; Flow rate: 25 mL/min; Gradient: 19% B to 22% B in 10 min; Wave Length: 254 nm; RT1(min): 7.27;) to afford 3-[(3-fluoro-2-methoxyphenyl)amino]-2-[3-(pyridin-2-ylmethoxy)pyridin-4-yl]-1H,5H,6H,7H-pyrrolo[3,2-c]pyridin-4-one (14.7 mg, 11.79%) as a light brown solid.

LC-MS: M+H found: 460.05.

¹H NMR (400 MHz, DMSO-d6) δ 12.30 (s, 1H), 8.72 (dt, J=4.7, 1.4 Hz, 1H), 8.45 (s, 1H), 8.03 (d, J=5.1 Hz, 1H), 7.91 (td, J=7.7, 1.8 Hz, 1H), 7.59-7.49 (m, 2H), 7.44 (m, 1H), 7.37 (d, J=5.1 Hz, 1H), 7.19 (t, J=2.6 Hz, 1H), 6.65 (td, J=8.3, 6.0 Hz, 1H), 6.52 (m, 1H), 6.05 (dt, J=8.2, 1.3 Hz, 1H), 5.55 (s, 2H), 3.91 (s, 3H), 3.47 (td, J=6.8, 2.5 Hz, 2H), 2.98 (t, J=6.8 Hz, 2H).

### Example 26. 3-[(3-fluoro-2-methoxyphenyl)amino]-2-[3-(1,3-oxazol-2-ylmethoxy)pyridin-4-yl]-1H,5H,6H,7H-pyrrolo[3,2-c]pyridin-4-one (compound 474)

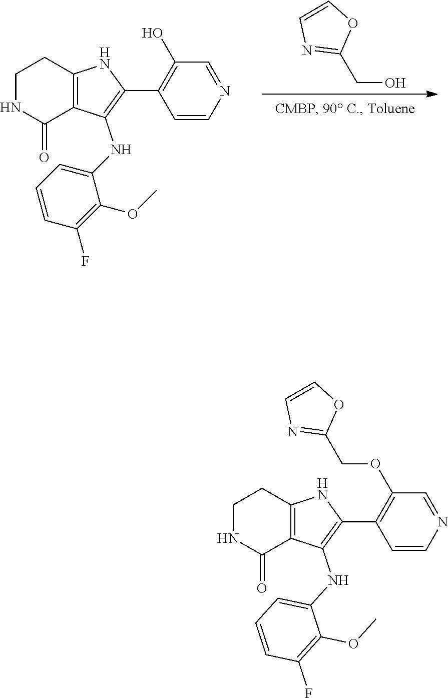 [SMILES: COc1c(F)cccc1Nc1c(-c2ccncc2O)[nH]c2c1C(=O)NCC2 ; OCc1ncco1 ; COc1c(F)cccc1Nc1c(-c2ccncc2OCc2ncco2)[nH]c2c1C(=O)NCC2]

A mixture of 3-[(3-fluoro-2-methoxyphenyl)amino]-2-(3-hydroxypyridin-4-yl)-1H,5H,6H,7H-pyrrolo[3,2-c]pyridin-4-one (300.00 mg, 0.814 mmol, 1.00 equiv), 2-(tributyl-lambda 5-phosphanylidene)acetonitrile (786.25 mg, 3.256 mmol, 4.00 equiv) and 1,3-oxazol-2-ylmethanol (161.40 mg, 1.628 mmol, 2.00 equiv) in Toluene (5.00 mL) was stirred for 4 h at 90 degrees C. under argon atmosphere. The resulting mixture was concentrated under reduced pressure. The resulting mixture was extracted with EtOAc (3×30 mL). The combined organic layers were washed with brine (2×30 mL), dried over anhydrous Na2SO4. After filtration, the filtrate was concentrated under reduced pressure. The crude product (30 mg) was purified by Prep-HPLC with the following conditions (Column: Xselect CSH C18 OBD Column 30*150 mm 5 μm; Mobile Phase A: Water (0.1% FA), Mobile Phase B: ACN; Flow rate: 60 mL/min; Gradient: 15% B to 26% B in 8 min; Wave Length: 254/220 nm; RT1(min): 7.65;) to afford 3-[(3-fluoro-2-methoxyphenyl)amino]-2-[3-(1,3-oxazol-2-ylmethoxy)pyridin-4-yl]-1H,5H,6H,7H-pyrrolo[3,2-c]pyridin-4-one (4.1 mg, 1.09%) as a white solid.

LC-MS: (M+H)+ found 450.05.

¹H NMR (400 MHz, DMSO-d6) δ 11.52 (s, 1H), 8.50 (s, 1H), 8.21 (s, 1H), 8.05 (d, J=5.1 Hz, 1H), 7.48 (s, 1H), 7.37-7.29 (m, 2H), 7.15 (d, J=2.7 Hz, 1H), 6.62 (td, J=8.3, 6.0 Hz, 1H), 6.48 (m, 1H), 5.99 (d, J=8.2 Hz, 1H), 5.53 (s, 2H), 3.90 (s, 3H), 3.44 (s, 2H), 2.89 (t, J=6.8 Hz, 2H).

### Example 27. 3-[(3-fluoro-2-methoxyphenyl)amino]-2-(3-[imidazo[1,2-a]pyridin-8-ylmethoxy]pyridin-4-yl)-1H,5H,6H,7H-pyrrolo[3,2-c]pyridin-4-one (compound 475)

### 27.1. Synthesis of 3-[imidazo[1,2-a]pyridin-8-ylmethoxy]pyridine-4-carbonitrile

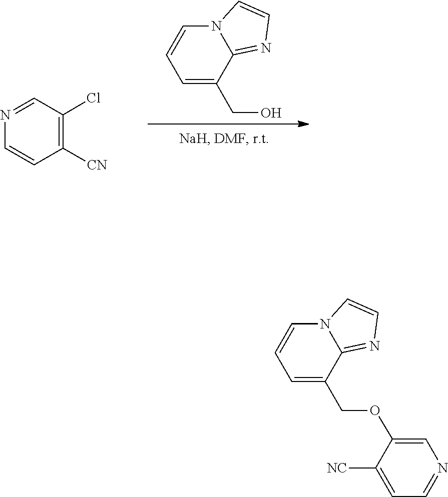 [SMILES: N#Cc1ccncc1Cl ; OCc1cccn2ccnc12 ; N#Cc1ccncc1OCc1cccn2ccnc12]

To a mixture of imidazo[1,2-a]pyridin-8-ylmethanol (149 mg, 1.01 mmol, 1.00 equiv) in DMF (2 mL) was added NaH (48 mg, 2.0 mmol, 2.00 equiv) at 0° C. and stirred for 0.5h at room temperature. To the mixture was added 3-chloropyridine-4-carbonitrile (140 mg, 1.01 mmol, 1.00 equiv) and was stirred for 1 h at room temperature under nitrogen atmosphere. The resulting mixture was extracted with EtOAc (3×15 mL). The combined organic layers were washed with brine (1×10 mL), dried over anhydrous Na2SO4. After filtration, the filtrate was concentrated under reduced pressure. The residue was purified by Prep-TLC (CH2Cl2/MeOH 15:1) to afford 3-[imidazo[1,2-a]pyridin-8-ylmethoxy]pyridine-4-carbonitrile (210 mg, 83.04%) as a light yellow solid.

LC-MS: (M+H)+ found: 251.2.

### 27.2. Synthesis of 1-(3-[imidazo[1,2-a]pyridin-8-ylmethoxy]pyridin-4-yl)methanamine

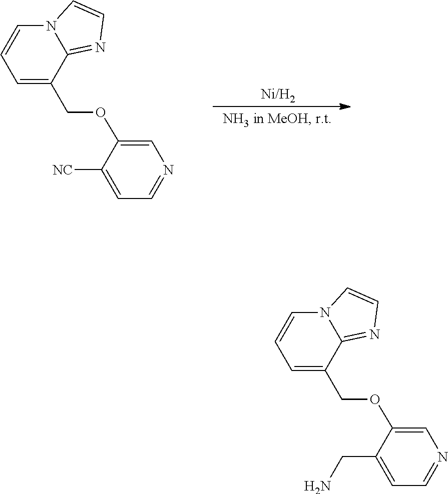 [SMILES: N#Cc1ccncc1OCc1cccn2ccnc12 ; NCc1ccncc1OCc1cccn2ccnc12]

A solution of 3-[imidazo[1,2-a]pyridin-8-ylmethoxy]pyridine-4-carbonitrile (210 mg, 0.83 mmol, 1.00 equiv) and Raney Ni (143 mg, 1.67 mmol, 2.00 equiv) in NH3 (5 mL, 7M in MeOH) and MeOH (5 mL) was stirred for 2h at room temperature under hydrogen atmosphere. The resulting mixture was filtered, the filter cake was washed with MeOH (1×10 mL). The filtrate was concentrated under reduced pressure. The residue was purified by Prep-TLC (CH2Cl2/MeOH 1:1) to afford 1-(3-[imidazo[1,2-a]pyridin-8-ylmethoxy]pyridin-4-yl)methanamine (90 mg, 42.18%) as a light yellow solid.

LC-MS: (M+H)+ found 255.2.

### 27.3. Synthesis of tert-butyl 3-[(3-fluoro-2-methoxyphenyl)carbamothioyl]-4-[[(3-[imidazo[1,2-a]pyridin-8-ylmethoxy]pyridin-4-yl)methyl]amino]-2-oxo-5,6-dihydropyridine-1-carboxylate

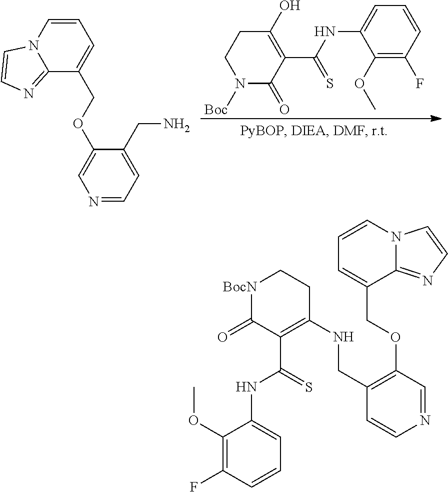 [SMILES: NCc1ccncc1OCc1cccn2ccnc12 ; COc1c(F)cccc1NC(=S)C1=C(O)CCN(C(=O)OC(C)(C)C)C1=O ; COc1c(F)cccc1NC(=S)C1=C(NCc2ccncc2OCc2cccn3ccnc23)CCN(C(=O)OC(C)(C)C)C1=O]

A solution of 1-(3-[imidazo[1,2-a]pyridin-8-ylmethoxy]pyridin-4-yl)methanamine (46 mg, 0.18 mmol, 1.00 equiv) and tert-butyl 3-[(3-fluoro-2-methoxyphenyl)carbamothioyl]-4-hydroxy-2-oxo-5,6-dihydropyridine-1-carboxylate (71 mg, 0.18 mmol, 1.00 equiv), PyBOP (113 mg, 0.21 mmol, 1.20 equiv), DIEA (47 mg, 0.36 mmol, 2.00 equiv) in DMF (1 mL) was stirred for overnight at room temperature under nitrogen atmosphere. The resulting mixture was extracted with EtOAc (2×10 mL). The combined organic layers were washed with brine (1×10 mL), dried over anhydrous Na2SO4. After filtration, the filtrate was concentrated under reduced pressure. The residue was purified by Prep-TLC (PE/EtOAc 1:1) to afford tert-butyl 3-[(3-fluoro-2-methoxyphenyl)carbamothioyl]-4-[[(3-[imidazo[1,2-a]pyridin-8-ylmethoxy]pyridin-4-yl)methyl]amino]-2-oxo-5,6-dihydropyridine-1-carboxylate (25 mg, 21.84%) as a light yellow oil.

LC-MS: (M+H)+ found 633.2.

### 27.4. Synthesis of 3-[(3-fluoro-2-methoxyphenyl)amino]-2-(3-[imidazo[1,2-a]pyridin-8-ylmethoxy]pyridin-4-yl)-1H,5H,6H,7H-pyrrolo[3,2-c]pyridin-4-one

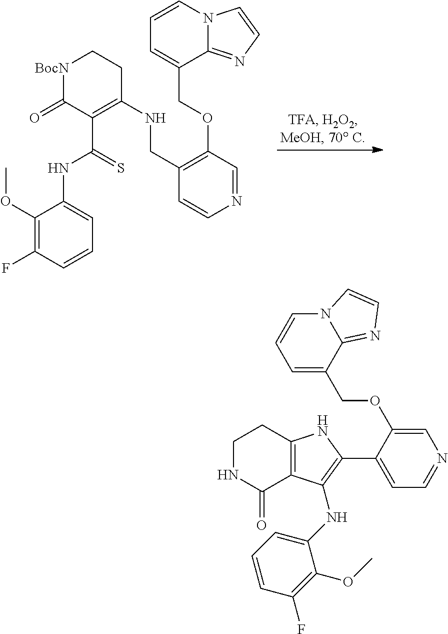 [SMILES: COc1c(F)cccc1NC(=S)C1=C(NCc2ccncc2OCc2cccn3ccnc23)CCN(C(=O)OC(C)(C)C)C1=O ; COc1c(F)cccc1Nc1c(-c2ccncc2OCc2cccn3ccnc23)[nH]c2c1C(=O)NCC2]

A solution of tert-butyl 3-[(3-fluoro-2-methoxyphenyl)carbamothioyl]-4-[[(3-[imidazo[1,2-a]pyridin-8-ylmethoxy]pyridin-4-yl)methyl]amino]-2-oxo-5,6-dihydropyridine-1-carboxylate (80 mg, 0.12 mmol, 1.00 equiv), TFA (57 mg, 0.50 mmol, 4.00 equiv) and H₂O₂ (30%) (8 mg, 0.25 mmol, 2.00 equiv) in MeOH (1 mL) was stirred for 1 h at 60° C. under nitrogen atmosphere. The reaction was quenched by the addition of sat. NaHSO3 (aq.) (1 mL) at room temperature. The resulting mixture was concentrated under reduced pressure. The residue was purified by reverse phase flash with the following conditions (MeCN/H₂O=40%) to afford 3-[(3-fluoro-2-methoxyphenyl)amino]-2-(3-[imidazo[1,2-a]pyridin-8-ylmethoxy]pyridin-4-yl)-1H,5H,6H,7H-pyrrolo[3,2-c]pyridin-4-one (5.5 mg, 8.73%) as an off-white solid.

LC-MS: (M+H)+ found 499.0

¹H NMR (300 MHz, DMSO-d6) δ 12.41 (s, 1H), 8.69-8.56 (m, 2H), 8.11 (d, J=1.3 Hz, 1H), 7.96 (d, J=5.0 Hz, 1H), 7.80 (d, J=1.3 Hz, 1H), 7.58 (d, J=7.3 Hz, 2H), 7.26 (d, J=5.0 Hz, 1H), 7.15 (d, J=2.6 Hz, 1H), 7.02 (t, J=6.8 Hz, 1H), 6.63 (td, J=8.3, 6.0 Hz, 1H), 6.49 (ddd, J=10.0, 8.4, 1.5 Hz, 1H), 6.01 (dt, J=8.2, 1.3 Hz, 1H), 5.70 (s, 2H), 3.91 (s, 3H), 3.41 (td, J=6.8, 2.5 Hz, 2H), 2.89 (t, J=6.8 Hz, 2H).

### Example 28. 3-[(3-fluoro-2-methoxyphenyl)amino]-2-[3-[(3-methoxypyridin-2-yl)methoxy]pyridin-4-yl]-1H,5H,6H,7H-pyrrolo[3,2-c]pyridin-4-one (compound 476)

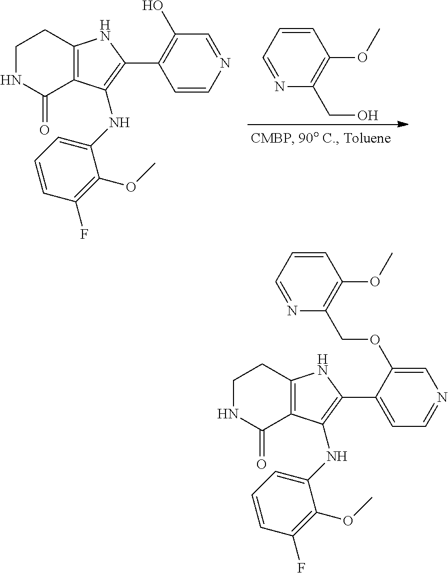

A solution of 3-[(3-fluoro-2-methoxyphenyl)amino]-2-(3-hydroxypyridin-4-yl)-1H,5H,6H,7H-pyrrolo[3,2-c]pyridin-4-one (100.00 mg, 0.271 mmol, 1.00 equiv) and (3-methoxypyridin-2-yl)methanol (75.55 mg, 0.542 mmol, 2.00 equiv) in Toluene (1.00 mL) was treated with 2-(tributyl-lambda 5-phosphanylidene)acetonitrile (131.04 mg, 0.542 mmol, 2.00 equiv). The mixture was stirred for overnight at 90 degrees C. under nitrogen atmosphere. The resulting mixture was concentrated under reduced pressure. The resulting mixture was diluted with 2 ml DMF. The crude product (100 mg) was purified by Prep-HPLC with the following conditions (Column: XBridge Prep OBD C18 Column, 30*150 mm, 5 μm; Mobile Phase A: Water (10 MMOL/L NH4HCO3), Mobile Phase B: ACN; Flow rate: 50 mL/min; Gradient: 35% B to 50% B in 8 min; Wave Length: 254 nm; RT1(min): 7.6;) to afford 3-[(3-fluoro-2-methoxyphenyl)amino]-2-[3-[(3-methoxypyridin-2-yl)methoxy]pyridin-4-yl]-1H,5H,6H,7H-pyrrolo[3,2-c]pyridin-4-one (6.6 mg, 4.90%) as a white solid.

LC-MS: (M+H)+ found 489.95

¹H NMR (400 MHz, DMSO-d6) δ 12.80 (s, 1H), 8.52 (s, 1H), 8.34 (dd, J=4.8, 1.2 Hz, 1H), 8.01 (d, J=5.1 Hz, 1H), 7.62 (dd, J=8.5, 1.2 Hz, 1H), 7.58 (s, 1H), 7.50 (dd, J=8.3, 4.8 Hz, 1H), 7.39 (d, J=5.1 Hz, 1H), 7.20 (t, J=2.5 Hz, 1H), 6.69 (td, J=8.3, 6.0 Hz, 1H), 6.55 m, 1H), 6.05 (dt, J=8.1, 1.2 Hz, 1H), 5.60 (s, 2H), 3.98-3.92 (m, 6H), 3.48 (td, J=6.8, 2.5 Hz, 2H), 3.03 (t, J=6.8 Hz, 2H).

### Example 29. 3-[(3-fluoro-2-methoxyphenyl)amino]-2-[3-[(5-methylpyrimidin-2-yl)methoxy]pyridin-4-yl]-1H,5H,6H,7H-pyrrolo[3,2-c]pyridin-4-one (compound 477)

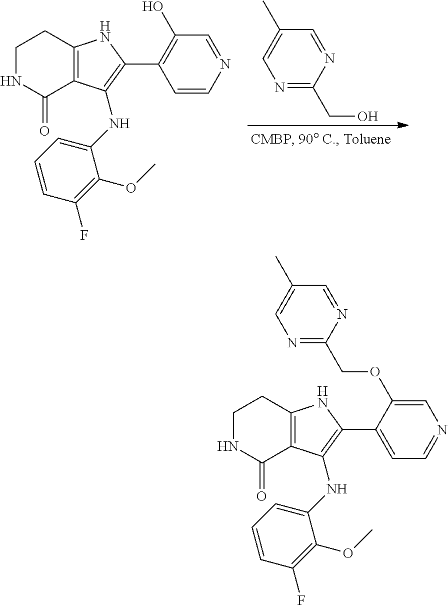

A mixture of 3-[(3-fluoro-2-methoxyphenyl)amino]-2-(3-hydroxypyridin-4-yl)-1H,5H,6H,7H-pyrrolo[3,2-c]pyridin-4-one (150.00 mg, 0.407 mmol, 1.00 equiv), 2-(tributyl-lambda 5-phosphanylidene)acetonitrile (786.25 mg, 3.256 mmol, 8.00 equiv) and (5-methylpyrimidin-2-yl)methanol (202.20 mg, 1.628 mmol, 4.00 equiv) in Toluene (5.00 mL) was stirred for 4 h at 90 degrees C. under argon atmosphere. The resulting mixture was concentrated under reduced pressure. The resulting mixture was extracted with EtOAc (3×30 mL). The combined organic layers were washed with brine (2×30 mL), dried over anhydrous Na2SO4. After filtration, the filtrate was concentrated under reduced pressure. The resulting mixture was diluted with DMF (2 mL). The crude product (30 mg) was purified by Prep-HPLC with the following conditions (Column: XBridge Prep OBD C18 Column, 30*150 mm, 5 μm; Mobile Phase A: Water (10 MMOL/L NH4HCO3), Mobile Phase B: ACN; Flow rate: 60 mL/min; Gradient: 32% B to 42% B in 8 min; Wave Length: 254 nm; RT1(min): 6;) to afford 3-[(3-fluoro-2-methoxyphenyl)amino]-2-[3-[(5-methylpyrimidin-2-yl)methoxy]pyridin-4-yl]-1H,5H,6H,7H-pyrrolo[3,2-c]pyridin-4-one (12.2 mg, 6.26%) as a light brown solid.

LC-MS: (M+H)+ found 475.35.

¹H NMR (400 MHz, DMSO-d6) δ 12.51 (s, 1H), 8.85 (s, 2H), 8.51 (s, 1H), 8.02 (d, J=5.2 Hz, 1H), 7.58 (s, 1H), 7.39 (d, J=5.2 Hz, 1H), 7.21 (t, J=2.6 Hz, 1H), 6.69 (td, J=8.3, 5.9 Hz, 1H), 6.60-6.51 (m, 1H), 6.05 (d, J=8.2 Hz, 1H), 5.64 (s, 2H), 3.95 (s, 3H), 3.48 (m, 2H), 3.03 (t, J=6.8 Hz, 2H), 2.36 (s, 3H).

### Example 30. 3-[(3-fluoro-2-methoxyphenyl)amino]-2-[3-(pyrimidin-4-ylmethoxy)pyridin-4-yl]-1H,5H,6H,7H-pyrrolo[3,2-c]pyridin-4-one (compound 478)

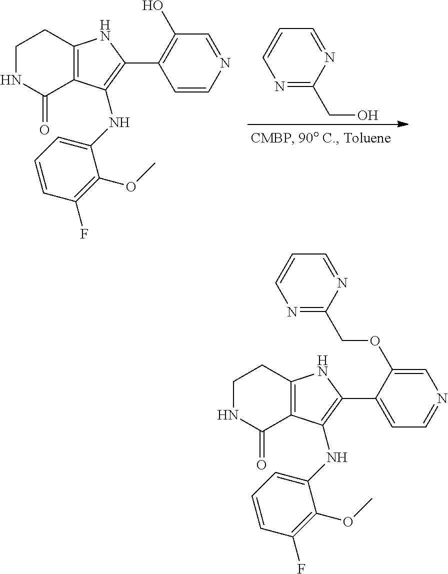

A mixture of 3-[(3-fluoro-2-methoxyphenyl)amino]-2-(3-hydroxypyridin-4-yl)-1H,5H,6H,7H-pyrrolo[3,2-c]pyridin-4-one (290.00 mg, 0.787 mmol, 1.00 equiv), 2-(tributyl-lambda 5-phosphanylidene)acetonitrile (380.02 mg, 1.575 mmol, 2 equiv) and pyrimidin-4-ylmethanol (173.38 mg, 1.575 mmol, 2.00 equiv) in Toluene (2.00 mL) was stirred for overnight at 90 degrees C. under argon atmosphere. The resulting mixture was concentrated under reduced pressure. The resulting mixture was extracted with EtOAc (3×30 mL). The combined organic layers were washed with brine (2×30 mL), dried over anhydrous Na2SO4. After filtration, the filtrate was concentrated under reduced pressure. The crude product (30 mg) was purified by Prep-HPLC with the following conditions (Column: XBridge Shield RP18 OBD Column, 30*150 mm, 5 μm; Mobile Phase A: Water (0.1% FA), Mobile Phase B: ACN; Flow rate: 60 mL/min; Gradient: 15% B to 30% B in 8 min; Wave Length: 254 nm; RT1(min): 6.2;) to afford 3-[(3-fluoro-2-methoxyphenyl)amino]-2-[3-(pyrimidin-4-ylmethoxy)pyridin-4-yl]-1H,5H,6H,7H-pyrrolo[3,2-c]pyridin-4-one (2.7 mg, 0.74%) as a light yellow solid.

LC-MS: (M+H)+ found 461.30.

¹H NMR (400 MHz, DMSO-d6) δ 11.88 (s, 1H), 9.30 (d, J=1.4 Hz, 1H), 8.88 (d, J=5.2 Hz, 1H), 8.39 (s, 1H), 8.06 (d, J=5.1 Hz, 1H), 7.62-7.53 (m, 2H), 7.37 (d, J=5.0 Hz, 1H), 7.19-7.13 (m, 1H), 6.64 (m, J=8.3, 6.0 Hz, 1H), 6.50 (m, J=10.9, 8.3, 1.5 Hz, 1H), 6.03 (m, J=8.2, 1.3 Hz, 1H), 5.53 (s, 2H), 3.88 (s, 3H), 3.46 (m, 2H), 2.96 (t, J=6.8 Hz, 2H).

### Example 31. 2-[3-(1,2,3-benzotriazol-1-ylmethoxy)pyridin-4-yl]-3-[(3-fluoro-2-methoxyphenyl)amino]-1H,5H,6H,7H-pyrrolo[3,2-c]pyridin-4-one (compound 479)

### 31.1. Synthesis of 1-(chloromethyl)-1,2,3-benzotriazole

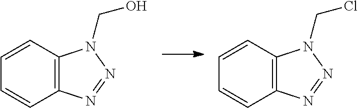 [SMILES: OCn1nnc2ccccc21 ; ClCn1nnc2ccccc21]

To a stirred mixture of 1,2,3-benzotriazol-1-ylmethanol (150.00 mg, 1.006 mmol, 1.00 equiv) in thionyl chloride (2.00 mL) was added a drop of DMF at room temperature under nitrogen atmosphere. The resulting mixture was stirred for 30 min at room temperature under nitrogen atmosphere. The resulting mixture was stirred for overnight at 50 degrees C. under nitrogen atmosphere. The resulting mixture was concentrated under reduced pressure.

LC-MS: (M+H)+ found 168.00.

### 31.2. Synthesis of 2-[3-(1,2,3-benzotriazol-1-ylmethoxy)pyridin-4-yl]-3-[(3-fluoro-2-methoxyphenyl)amino]-1H,5H,6H,7H-pyrrolo[3,2-c]pyridin-4-one

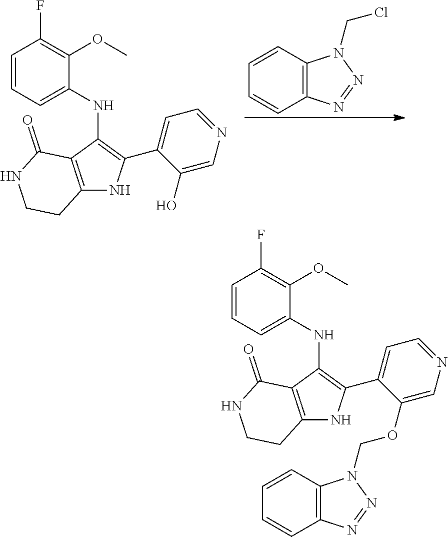

A mixture of 3-[(3-fluoro-2-methoxyphenyl)amino]-2-(3-hydroxypyridin-4-yl)-1H,5H,6H,7H-pyrrolo[3,2-c]pyridin-4-one (200.00 mg, 0.543 mmol, 1.00 equiv), 1-(chloromethyl)-1,2,3-benzotriazole (136.49 mg, 0.815 mmol, 1.50 equiv) and Na₂CO₃ (115.09 mg, 1.086 mmol, 2.00 equiv) in DMF (5.00 mL) was stirred for 2 h at room temperature under argon atmosphere. The resulting mixture was extracted with EtOAc (3×30 mL). The combined organic layers were washed with brine (2×30 mL), dried over anhydrous Na₂SO₄. After filtration, the filtrate was concentrated under reduced pressure. The resulting mixture was diluted with DMF (2 mL). The crude product (30 mg) was purified by Prep-HPLC with the following conditions (Column: XBridge Shield RP18 OBD Column, 30*150 mm, 5 μm; Mobile Phase A: Water (0.1% FA), Mobile Phase B: ACN; Flow rate: 60 mL/min; Gradient: 15% B to 30% B in 8 min; Wave Length: 254 nm; RT1(min): 6.2;) to afford 2-[3-(1,2,3-benzotriazol-1-ylmethoxy)pyridin-4-yl]-3-[(3-fluoro-2-methoxyphenyl)amino]-1H,5H,6H,7H-pyrrolo[3,2-c]pyridin-4-one (15.4 mg, 5.68%) as a light yellow solid.

LC-MS: (M+H)+ found 500.10.

¹H NMR (400 MHz, DMSO-d6) δ 11.16 (s, 1H), 8.68 (s, 1H), 8.05 (d, J=5.1 Hz, 1H), 7.99 (dt, J=8.5, 0.9 Hz, 1H), 7.90 (dt, J=8.3, 1.0 Hz, 1H), 7.53 (m, 1H), 7.38 (m, 1H), 7.30 (s, 1H), 7.19 (d, J=5.1 Hz, 1H), 7.11-7.06 (m, 1H), 6.85 (s, 2H), 6.49-6.39 (m, 2H), 5.33-5.24 (m, 1H), 3.86 (d, J=0.8 Hz, 3H), 3.38-3.30 (m, 2H), 2.78 (t, J=6.8 Hz, 2H).

### Example 32. 3-[(3-fluoro-2-methoxyphenyl)amino]-2-[3-[(2-methylpyrazol-3-yl)methoxy]pyridin-4-yl]-1H,5H,6H,7H-pyrrolo[3,2-c]pyridin-4-one (compound 480)

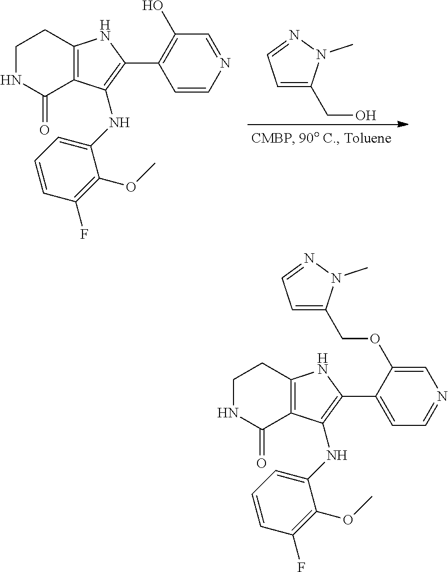

A mixture of 3-[(3-fluoro-2-methoxyphenyl)amino]-2-(3-hydroxypyridin-4-yl)-1H,5H,6H,7H-pyrrolo[3,2-c]pyridin-4-one (150.00 mg, 0.407 mmol, 1.00 equiv), 2-(tributyl-lambda 5-phosphanylidene)acetonitrile (393.13 mg, 1.628 mmol, 4.00 equiv) and (2-methylpyrazol-3-yl)methanol (365.28 mg, 3.256 mmol, 8.00 equiv) in Toluene (5.00 mL) was stirred for 4 h at 90 degrees C. under argon atmosphere. The resulting mixture was concentrated under reduced pressure. The resulting mixture was extracted with EtOAc (3×30 mL). The combined organic layers were washed with brine (2×30 mL), dried over anhydrous Na₂SO₄. After filtration, the filtrate was concentrated under reduced pressure. The resulting mixture was diluted with DMF (2 mL). The crude product (30 mg) was purified by Prep-HPLC with the following conditions (Column: XSelect CSH Fluoro Phenyl, 30*150 mm, 5 μm; Mobile Phase A: Water (0.05% FA), Mobile Phase B: ACN; Flow rate: 60 mL/min; Gradient: 17% B to 20% B in 10 min; Wave Length: 254 nm; RT1(min): 7.62) to afford 3-[(3-fluoro-2-methoxyphenyl)amino]-2-[3-[(2-methylpyrazol-3-yl)methoxy]pyridin-4-yl]-1H,5H,6H,7H-pyrrolo[3,2-c]pyridin-4-one (14.8 mg, 4.52%) as a light yellow solid.

LC-MS: M+H found: 463.10

¹H NMR (400 MHz, DMSO-d6) δ 11.25 (s, 1H), 8.54 (s, 1H), 8.05 (d, J=5.0 Hz, 1H), 7.39-7.34 (m, 2H), 7.28 (d, J=5.0 Hz, 1H), 7.10 (d, J=2.6 Hz, 1H), 6.55 (td, J=8.2, 6.0 Hz, 1H), 6.44 (m, J=10.9, 8.3, 1.5 Hz, 1H), 6.33 (d, J=1.9 Hz, 1H), 5.87 (m, J=8.2, 1.3 Hz, 1H), 5.41 (s, 2H), 3.84 (s, 3H), 3.75 (s, 3H), 3.50-3.35 (m, 2H), 2.83 (t, J=6.8 Hz, 2H).

### Example 33. 3-[(3-fluoro-2-methoxyphenyl)amino]-2-(3-[imidazo[1,2-a]pyridin-2-ylmethoxy]pyridin-4-yl)-1H,5H,6H,7H-pyrrolo[3,2-c]pyridin-4-one (compound 481)

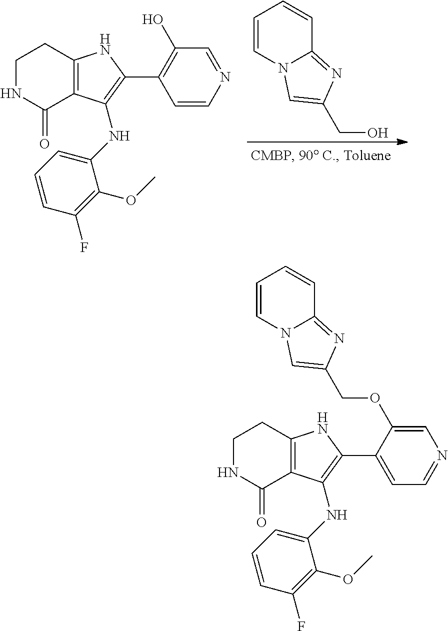

A mixture of bis(3-[(3-fluoro-2-methoxyphenyl)amino]-2-(3-hydroxypyridin-4-yl)-1H,5H,6H,7H-pyrrolo[3,2-c]pyridin-4-one) (220.00 mg, 0.299 mmol, 1.00 equiv), 2-(tributyl-lambda 5-phosphanylidene)acetonitrile (288.29 mg, 1.196 mmol, 4.00 equiv) and imidazo[1,2-a]pyridin-2-ylmethanol (88.49 mg, 0.598 mmol, 2.00 equiv) in Toluene (5.00 mL) was stirred for overnight at 90 degrees C. under argon atmosphere. The resulting mixture was concentrated under reduced pressure. The resulting mixture was extracted with EtOAc (3×30 mL). The combined organic layers were washed with brine (2×30 mL), dried over anhydrous Na₂SO₄. After filtration, the filtrate was concentrated under reduced pressure. The crude product (30 mg) was purified by Prep-HPLC with the following conditions (Column: XSelect CSH Fluoro Phenyl, 30*150 mm, 5 μm; Mobile Phase A: Water (0.05% FA), Mobile Phase B: ACN; Flow rate: 60 mL/min; Gradient: 17% B to 20% B in 10 min; Wave Length: 254 nm; RT1(min): 7.62) to afford 3-[(3-fluoro-2-methoxyphenyl)amino]-2-(3-[imidazo[1,2-a]pyridin-2-ylmethoxy]pyridin-4-yl)-1H,5H,6H,7H-pyrrolo[3,2-c]pyridin-4-one (13.7 mg, 9.09%) as a yellow solid.

LC-MS: (M+H)+ found 499.10.

¹H NMR (400 MHz, DMSO-d6) δ 12.15 (s, 1H), 8.60 (d, J=6.0 Hz, 2H), 8.07-7.98 (m, 2H), 7.64 (dd, J=35.9, 9.1 Hz, 1H), 7.53 (s, 1H), 7.33 (dd, J=8.3, 5.4 Hz, 2H), 7.15 (d, J=2.6 Hz, 1H), 6.96 (td, J=6.8, 1.2 Hz, 1H), 6.62 (td, J=8.3, 6.0 Hz, 1H), 6.49 (m, J=10.0, 8.4, 1.5 Hz, 1H), 6.04 (d, J=8.2 Hz, 1H), 5.57 (s, 2H), 3.91 (s, 3H), 3.46 (m, 2H), 3.00 (t, J=6.8 Hz, 2H).

### Example 34. 3-[(3-fluoro-2-methoxyphenyl)amino]-2-[3-[(1-methylimidazol-2-yl)methoxy]pyridin-4-yl]-1H,5H,6H,7H-pyrrolo[3,2-c]pyridin-4-one (compound 482)

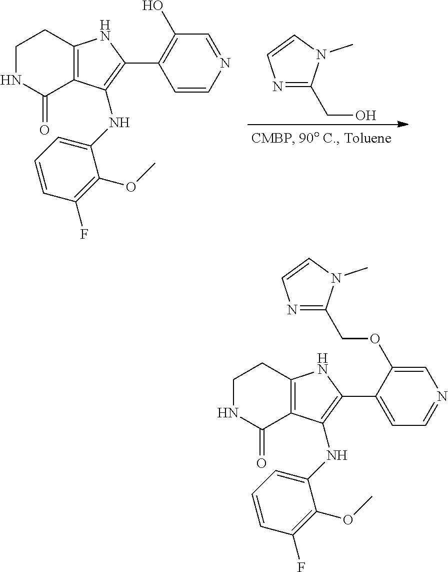

A mixture of 3-[(3-fluoro-2-methoxyphenyl)amino]-2-(3-hydroxypyridin-4-yl)-1H,5H,6H,7H-pyrrolo[3,2-c]pyridin-4-one (300.00 mg, 0.814 mmol, 1.00 equiv), 2-(tributyl-lambda 5-phosphanylidene)acetonitrile (393.13 mg, 1.629 mmol, 2.00 equiv) and (1-methylimidazol-2-yl)methanol (182.64 mg, 1.629 mmol, 2.00 equiv) in Toluene (5.00 mL) was stirred for overnight at 90 degrees C. under argon atmosphere. The resulting mixture was concentrated under reduced pressure. The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by Prep-TLC (DCM/MeOH 10:1) to afford 3-[(3-fluoro-2-methoxyphenyl)amino]-2-[3-[(1-methylimidazol-2-yl)methoxy]pyridin-4-yl]-1H,5H,6H,7H-pyrrolo[3,2-c]pyridin-4-one (30 mg, 7.71%) as a black solid. The resulting mixture was diluted with DMF (2 mL). The crude product (30 mg) was purified by Prep-HPLC with the following conditions (Column: Xcelect CSH F-pheny OBD Column, 19*250 mm, 5 μm; Mobile Phase A: Water (0.05% FA), Mobile Phase B: ACN; Flow rate: 25 mL/min; Gradient: 2% B to 18% B in 9 min; Wave Length: 254 nm; RT1(min): 8.77;) to afford 3-[(3-fluoro-2-methoxyphenyl)amino]-2-[3-[(1-methylimidazol-2-yl)methoxy]pyridin-4-yl]-1H,5H,6H,7H-pyrrolo[3,2-c]pyridin-4-one (5 mg, 1.29%) as a light yellow solid.

LC-MS: (M+Na)+ found 485.20.

¹H NMR (400 MHz, DMSO-d6) δ 12.60 (s, 1H), 8.58 (s, 1H), 8.03 (d, J=5.1 Hz, 1H), 7.48 (s, 1H), 7.32 (d, J=5.0 Hz, 1H), 7.22 (d, J=1.2 Hz, 1H), 7.16-7.11 (m, 1H), 7.00 (d, J=1.2 Hz, 1H), 6.64 (m, 1H), 6.50 (m, 1H), 6.00 (dt, J=8.2, 1.2 Hz, 1H), 5.53 (s, 2H), 3.91 (d, J=0.7 Hz, 3H), 3.66 (s, 3H), 3.44 (td, J=6.9, 2.5 Hz, 2H), 2.92 (t, J=6.8 Hz, 2H).

### Example 35. 3-[(3-fluoro-2-methoxyphenyl)amino]-2-[3-[2-(furan-2-yl)ethoxy]pyridin-4-yl]-1H,5H,6H,7H-pyrrolo[3,2-c]pyridin-4-one (compound 483)

### 35.1. Synthesis of 3-[2-(furan-2-yl)ethoxy]pyridine-4-carbonitrile

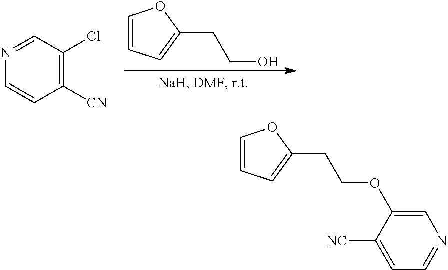

To a mixture of 2-(furan-2-yl)ethanol (112 mg, 1.00 mmol, 1.00 equiv) in DMF (2 mL) was added NaH (48 mg, 2.00 mmol, 2.00 equiv) at 0° C. and stirred for 0.5 h. To the mixture was added 3-chloropyridine-4-carbonitrile (139 mg, 1.00 mmol, 1.00 equiv) and stirred for 2h at room temperature under nitrogen atmosphere. The reaction was quenched with Water at room temperature. The resulting mixture was extracted with EtOAc (3×15 mL). The combined organic layers were washed with brine (1×10 mL), dried over anhydrous Na₂SO₄. After filtration, the filtrate was concentrated under reduced pressure. The residue was purified by Prep-TLC (DCM/MeOH 20:1) to afford 3-[2-(furan-2-yl)ethoxy]pyridine-4-carbonitrile (172 mg, 80.03%) as a light yellow solid.

LC-MS: (M+H)+ found: 215.2.

### 35.2. Synthesis of 1-[3-[2-(furan-2-yl)ethoxy]pyridin-4-yl]methanamine

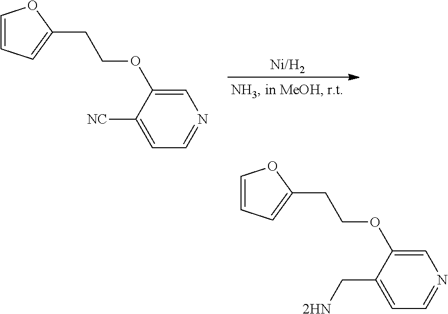

A solution of 3-[2-(furan-2-yl)ethoxy]pyridine-4-carbonitrile (172 mg, 0.80 mmol, 1.00 equiv) and Raney Ni (275 mg, 3.21 mmol, 4.00 equiv) in NH₃ (5 mL, 7M in MEOH) and MeOH (5 mL) was stirred for 2h at room temperature under hydrogen atmosphere. The resulting mixture was filtered, the filter cake was washed with MeOH (1×10 mL). The filtrate was concentrated under reduced pressure. The residue was purified by Prep-TLC (DCM/MeOH 1:1) to afford 1-[3-[2-(furan-2-yl)ethoxy]pyridin-4-yl]methanamine (130 mg, 74.19%) as a light yellow oil.

LC-MS: (M+H)+ found 219.1.

### 35.3. Synthesis of tert-butyl 3-[(3-fluoro-2-methoxyphenyl)carbamothioyl]-4-[([3-[2-(furan-2-yl)ethoxy]pyridin-4-yl]methyl)amino]-2-oxo-5,6-dihydropyridine-1-carboxylate

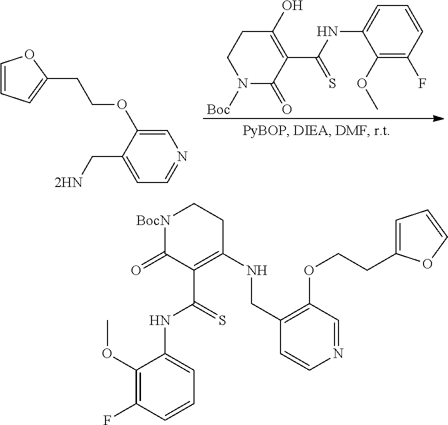

A solution of 1-[3-[2-(furan-2-yl)ethoxy]pyridin-4-yl]methanamine (120 mg, 0.55 mmol, 1.00 equiv) and tert-butyl 3-[(3-fluoro-2-methoxyphenyl)carbamothioyl]-4-hydroxy-2-oxo-5,6-dihydropyridine-1-carboxylate (217 mg, 0.55 mmol, 1.00 equiv), PyBOP (371 mg, 0.71 mmol, 1.30 equiv), DIEA (213 mg, 1.65 mmol, 3.00 equiv) in DMF (5 mL) was stirred for overnight at room temperature under nitrogen atmosphere. The resulting mixture was extracted with EtOAc (3×20 mL). The combined organic layers were washed with brine (1×10 mL), dried over anhydrous Na₂SO₄. After filtration, the filtrate was concentrated under reduced pressure. The residue was purified by Prep-TLC (PE/EtOAc 1:1) to afford tert-butyl 3-[(3-fluoro-2-methoxyphenyl)carbamothioyl]-4-[([3-[2-(furan-2-yl)ethoxy]pyridin-4-yl]methyl)amino]-2-oxo-5,6-dihydropyridine-1-carboxylate (75 mg, 22.86%) as a yellow oil.

LC-MS: (M+H)+ found 597.2.

### 35.4. Synthesis of 3-[(3-fluoro-2-methoxyphenyl)amino]-2-[3-[2-(furan-2-yl)ethoxy]pyridin-4-yl]-1H,5H,6H,7H-pyrrolo[3,2-c]pyridin-4-one

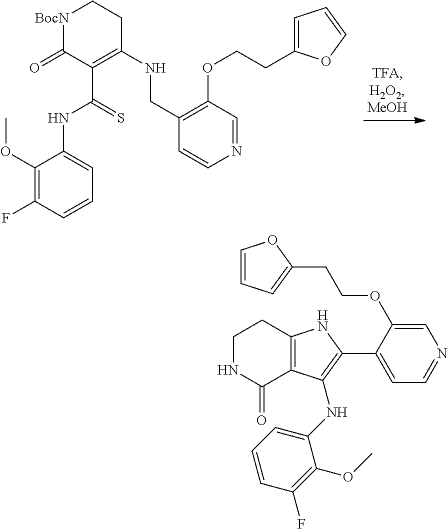 [SMILES: COc1c(F)cccc1NC(=S)C1=C(NCc2ccncc2OCCc2ccco2)CCN(C(=O)OC(C)(C)C)C1=O ; COc1c(F)cccc1Nc1c(-c2ccncc2OCCc2ccco2)[nH]c2c1C(=O)NCC2]

A solution of tert-butyl 3-[(3-fluoro-2-methoxyphenyl)carbamothioyl]-4-[([3-[2-(furan-2-yl)ethoxy]pyridin-4-yl]methyl)amino]-2-oxo-5,6-dihydropyridine-1-carboxylate (75 mg, 0.12 mmol, 1.00 equiv) and H₂O₂ (30%) (8 mg, 0.25 mmol, 2.00 equiv), TFA (57 mg, 0.50 mmol, 4.00 equiv) in was stirred for 2h at 60° C. under nitrogen atmosphere. The reaction was quenched with sat. NaHSO3 (aq.) at room temperature. The resulting mixture was concentrated under reduced pressure. The residue was purified by reverse phase flash with the following conditions (MeCN/H₂O=45%) to afford 3-[(3-fluoro-2-methoxyphenyl)amino]-2-[3-[2-(furan-2-yl)ethoxy]pyridin-4-yl]-1H,5H,6H,7H-pyrrolo[3,2-c]pyridin-4-one (7.7 mg, 13.25%) as a yellow solid.

LC-MS: (M+H)+ found 463.1

¹H NMR (300 MHz, DMSO-d6) δ 10.91 (s, 1H), 8.39 (s, 1H), 8.04 (d, J=5.0 Hz, 1H), 7.58 (d, J=1.9 Hz, 1H), 7.51 (s, 1H), 7.31 (d, J=5.0 Hz, 1H), 7.13 (s, 1H), 6.61 (td, J=8.3, 6.0 Hz, 1H), 6.53-6.42 (m, 1H), 6.40 (dd, J=3.2, 1.9 Hz, 1H), 6.26 (d, J=3.2 Hz, 1H), 5.96 (d, J=8.2 Hz, 1H), 4.39 (t, J=6.7 Hz, 2H), 3.90 (s, 3H), 3.42 (dt, J=6.5, 4.0 Hz, 2H), 3.21 (t, J=6.6 Hz, 2H), 2.86 (t, J=6.8 Hz, 2H).

### Example 36. 3-[(3-fluoro-2-methylphenyl)amino]-2-(3-[[(2S)-1-(prop-2-enoyl)pyrrolidin-2-yl]methoxy]pyridin-4-yl)-1H,5H,6H,7H-pyrrolo[3,2-c]pyridin-4-one (compound 467)

### 36.1. Synthesis of tert-butyl (2S)-2-[[(4-cyanopyridin-3-yl)oxy]methyl]pyrrolidine-1-carboxylate

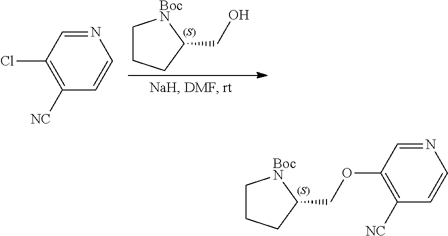

To a stirred solution/mixture of tert-butyl (2S)-2-(hydroxymethyl)pyrrolidine-1-carboxylate (5.00 g, 24.843 mmol, 1.00 equiv) in DMF (43.03 mL, 588.651 mmol, 22.38 equiv) was added NaH (1.19 g, 29.811 mmol, 1.20 equiv, 60%) dropwise/in portions at 0° C. under N₂ atmosphere. The resulting mixture was stirred for additional 0.5 h at 0° C. Then the 3-chloropyridine-4-carbonitrile (4.13 g, 29.811 mmol, 1.20 equiv) was added to the mixture. The mixture was stirred at 25° C. for 10 h. The reaction was quenched by the addition of H₂O (100 mL) at 0° C. The resulting mixture was extracted with EA (50 mL×3). The combined organic layers were washed with wine (30 mL×3), dried over anhydrous Na₂SO₄. After filtration, the filtrate was concentrated under reduced pressure. The residue was purified by silica gel column chromatography, eluted with PE:EA (2:1 ˜ 1:1) to afford tert-butyl (2S)-2-[[(4-cyanopyridin-3-yl)oxy]methyl]pyrrolidine-1-carboxylate (3.4 mg, 0.04%) as a yellow oil.

LC-MS: M-56+H found: 248.

### 36.2. Synthesis of tert-butyl (2S)-2-([[4-(aminomethyl)pyridin-3-yl]oxy]methyl)pyrrolidine-1-carboxylate

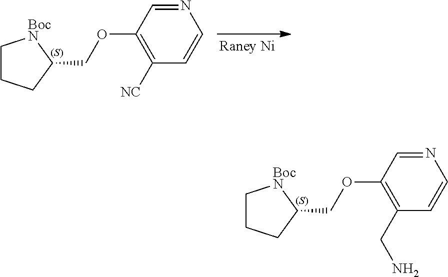

A solution of tert-butyl (2S)-2-[[(4-cyanopyridin-3-yl)oxy]methyl]pyrrolidine-1-carboxylate (3.00 g, 9.889 mmol, 1.00 equiv) in NH₃ (g) in MeOH (50.00 mL) was added Raney Ni (0.08 g, 0.989 mmol, 0.1 equiv). The mixture was treated with H2. The mixture was stirred at 25° C. for 10 h. The resulting mixture was filtered, the filter cake was washed with MeOH (20 mL×3). The filtrate was concentrated under reduced pressure. The residue was purified by silica gel column chromatography, eluted with DCM:MeOH (10:1 ˜ 3:1) to afford tert-butyl (2S)-2-([[4-(aminomethyl)pyridin-3-yl]oxy]methyl)pyrrolidine-1-carboxylate (1.9 g, 62.50%) as a yellow oil.

LC-MS: (M+H)+ found: 308.0.

### 36.3. Synthesis of tert-butyl (2S)-2-[([4-[([3-[(3-fluoro-2-methylphenyl)carbamothioyl]-2-oxo-5,6-dihydro-1H-pyridin-4-yl]oxy)methyl]pyridin-3-yl]oxy)methyl]pyrrolidine-1-carboxylate

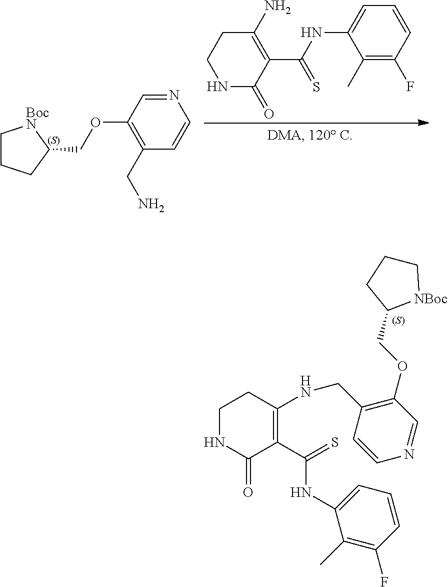

To a stirred solution/mixture of N-(3-fluoro-2-methylphenyl)-4-hydroxy-2-oxo-5,6-dihydro-1H-pyridine-3-carbothioamide (600.00 mg, 2.140 mmol, 1.00 equiv) and tert-butyl (2S)-2-([[4-(aminomethyl)pyridin-3-yl]oxy]methyl)pyrrolidine-1-carboxylate (986.92 mg, 3.211 mmol, 1.50 equiv) in DMA (2.40 mL, 27.549 mmol, 12.06 equiv) was stirred at 120° C. for 3 h. The resulting mixture was extracted with EA (30 mL×3). The combined organic layers were washed with wine (30 mL×3), dried over anhydrous Na₂SO₄. After filtration, the filtrate was concentrated under reduced pressure. The residue was purified by silica gel column chromatography, eluted with DCM:MeOH (20:1 ˜ 10:1) to afford tert-butyl (2S)-2-[([4-[([3-[(3-fluoro-2-methylphenyl)carbamothioyl]-2-oxo-5,6-dihydro-1H-pyridin-4-yl]oxy)methyl]pyridin-3-yl]oxy)methyl]pyrrolidine-1-carboxylate (400 mg, 32.75%) as a yellow solid.

LC-MS: (M+H)+ found 570.

### 36.4. Synthesis of tert-butyl (2S)-2-[[(4-[3-[(3-fluoro-2-methylphenyl)amino]-4-oxo-1H,5H,6H,7H-pyrrolo[3,2-c]pyridin-2-yl]pyridin-3-yl)oxy]methyl]pyrrolidine-1-carboxylate

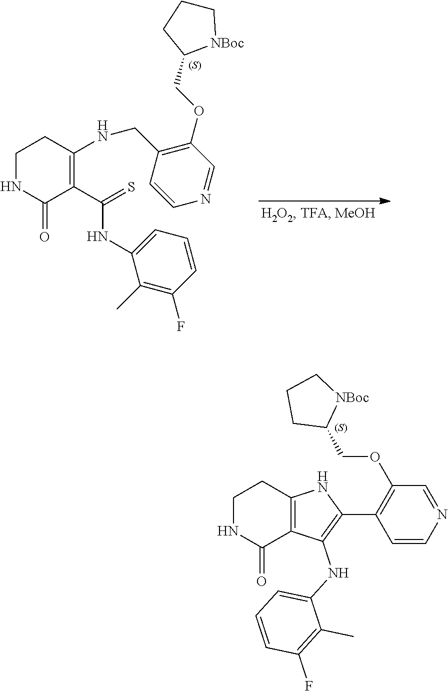

To a stirred solution/mixture of tert-butyl (2S)-2-[([4-[([3-[(3-fluoro-2-methylphenyl)carbamothioyl]-2-oxo-5,6-dihydro-1H-pyridin-4-yl]amino)methyl]pyridin-3-yl]oxy)methyl]pyrrolidine-1-carboxylate (400.00 mg, 0.702 mmol, 1.00 equiv) in MeOH (20.00 mL, 624.184 mmol, 703.55 equiv) were added H₂O₂ (300%) (398.04 mg, 3.510 mmol, 5.00 equiv, 30%) and TFA (200.15 mg, 1.755 mmol, 2.50 equiv) dropwise. The mixture was stirred at 80° C. for 3 h. The resulting mixture was concentrated under reduced pressure. The residue was purified by silica gel column chromatography, eluted with DCM:MeOH (30:1 ˜ 10:1) to afford tert-butyl (2S)-2-[[(4-[3-[(3-fluoro-2-methylphenyl)amino]-4-oxo-1H,5H,6H,7H-pyrrolo[3,2-c]pyridin-2-yl]pyridin-3-yl)oxy]methyl]pyrrolidine-1-carboxylate (100 mg, 26.59%) as a yellow solid.

LC-MS: (M+H)+ found 536.

### 36.5. Synthesis of 3-[(3-fluoro-2-methylphenyl)amino]-2-[3-[(2S)-pyrrolidin-2-ylmethoxy]pyridin-4-yl]-1H,5H,6H,711-pyrrolo[3,2-c]pyridin-4-one

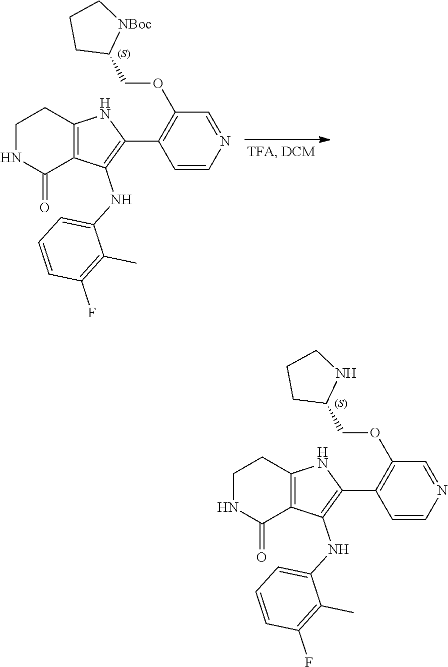

To a stirred solution/mixture of tert-butyl (2S)-2-[[(4-[3-[(3-fluoro-2-methylphenyl)amino]-4-oxo-1H,5H,6H,7H-pyrrolo[3,2-c]pyridin-2-yl]pyridin-3-yl)oxy]methyl]pyrrolidine-1-carboxylate (100 mg, 1 equiv) in DCM (6 mL) was added TFA (2 mL) dropwise at 25° C. The mixture was stirred at 25° C. for 2 h. The resulting mixture was concentrated under vacuum. The mixture/residue was acidified to pH 8-10 with a. q. NaHCO₃ (20 mL). The aqueous layer was extracted with EA (30 mL×3). The combined organic was concentrated under vacuum to afford 3-[(3-fluoro-2-methylphenyl)amino]-2-[3-[(2S)-pyrrolidin-2-ylmethoxy]pyridin-4-yl]-1H,5H,6H,7H-pyrrolo[3,2-c]pyridin-4-one (70 mg, 86.09%) as a yellow solid.

LC-MS: (M+H)+ found 436.

### 36.6. Synthesis of 3-[(3-fluoro-2-methylphenyl)amino]-2-(3-[[(2S)-1-(prop-2-enoyl)pyrrolidin-2-yl]methoxy]pyridin-4-yl)-1H,5H,6H,7H-pyrrolo[3,2-c]pyridin-4-one

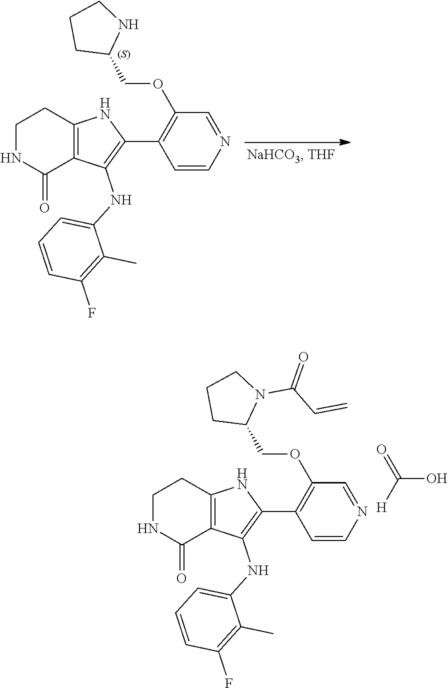

To a mixture of 3-[(3-fluoro-2-methylphenyl)amino]-2-[3-[(2S)-pyrrolidin-2-ylmethoxy]pyridin-4-yl]-1H,5H,6H,7H-pyrrolo[3,2-c]pyridin-4-one (100.00 mg, 0.230 mmol, 1.00 equiv) in THF (5.00 mL, 61.715 mmol, 109.44 equiv) and NaHCO₃ (5 mL) was added acryloyl chloride (41.57 mg, 0.459 mmol, 2 equiv) dropwise at 0° C. The mixture was stirred at 0° C. for 1 h. The resulting mixture was extracted with EA (30 ml×3). After filtration, the filtrate was concentrated under reduced pressure. The residue was purified by Prep-TLC (PE:EA=10:1) to afford the crude product. The crude product was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 15 B to 35 B in 8 min, 35 B to B in min, B to B in min, B to B in min, B to B in min; 254/220 nm) to afford 3-[(3-fluoro-2-methylphenyl)amino]-2-(3-[[(2S)-1-(prop-2-enoyl)pyrrolidin-2-yl]methoxy]pyridin-4-yl)-1H,5H,6H,7H-pyrrolo[3,2-c]pyridin-4-one (14.8 mg, 12.51%) as a yellow solid.

LC-MS: (M+H)+ found 490.

¹H NMR (400 MHz, DMSO-d6) δ 11.29 (s, 1H), 8.37 (s, 1H), 7.96 (s, 1H), 7.29 (d, J=4.8 Hz, 1H), 7.18 (s, 1H), 6.84 (s, 1H), 6.73 (d, J=7.6 Hz, 1H), 6.63 (dd, J=16.7, 10.4 Hz, 1H), 6.44 (t, J=8.8 Hz, 1H), 6.20 (d, J=17.2 Hz, 1H), 6.11 (d, J=8.2 Hz, 1H), 5.70 (s, 1H), 4.65 (s, 1H), 4.34 (s, 1H), 4.18 (s, 1H), 3.64 (s, 2H), 3.49-3.44 (m, 2H), 2.98 (s, 2H), 2.21 (s, 3H), 2.03 (m, 2H), 1.93 (m, 3H).

### Example 37. 3-[(3-fluoro-2-methylphenyl)amino]-2-(3-[[(2R)-1-(prop-2-enoyl)piperidin-2-yl]methoxy]pyridin-4-yl)-1H,5H,6H,7H-pyrrolo[3,2-c]pyridin-4-one (compound 468)

### 37.1. Synthesis of tert-butyl (2R)-2-[[(4-cyanopyridin-3-yl)oxy]methyl]piperidine-1-carboxylate

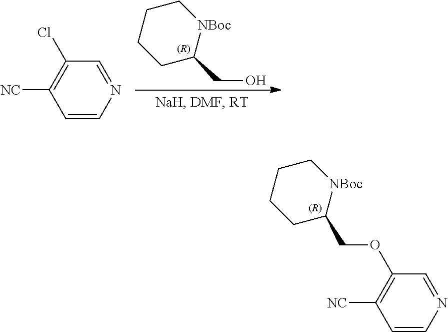

To a solution of 3-chloropyridine-4-carbonitrile (2.00 g, 14.435 mmol, 1.00 equiv), tert-butyl (2R)-2-(hydroxymethyl)piperidine-1-carboxylate (3.73 g, 17.322 mmol, 1.2 equiv) in DMF (40.00 mL) was added NaH (692.82 mg, 17.322 mmol, 1.2 equiv, 60%) at 0 degrees C. The mixture was stirred for 12h at rt. The reaction mixture was quenched by water (100 mL) and extracted with EA (3*100 mL). The combined organic layers were washed with brine (2*30 mL), dried over anhydrous Na₂SO₄. After filtration, the filtrate was concentrated under reduced pressure. The residue was purified by silica gel column chromatography, eluted with PE:EA (1:1) to afford tert-butyl (2R)-2-[[(4-cyanopyridin-3-yl)oxy]methyl]piperidine-1-carboxylate (2.5 g, 54.57%) as a brown oil.

LC-MS: M+H found: 318.

### 37.2. Synthesis of tert-butyl (2R)-2-([[4-(aminomethyl)pyridin-3-yl]oxy]methyl)piperidine-1-carboxylate

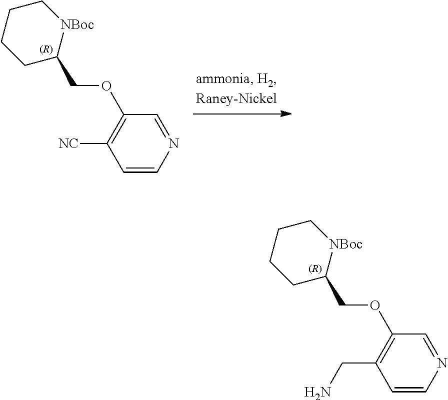

To a solution of tert-butyl (2R)-2-[[(4-cyanopyridin-3-yl)oxy]methyl]piperidine-1-carboxylate (2.50 g, 7.877 mmol, 1.00 equiv) in NH₃(g) in MeOH (50.00 mL) was added Raney Nickel (0.92 g, 15.754 mmol, 2.00 equiv) under nitrogen atmosphere. The mixture was hydrogenated at room temperature for 12h under hydrogen atmosphere using a hydrogen balloon, filtered through a Celite pad and concentrated under reduced pressure. The residue was purified by silica gel column chromatography, eluted with DCM:MeOH (8:1) to afford tert-butyl (2R)-2-([[4-(aminomethyl)pyridin-3-yl]oxy]methyl)piperidine-1-carboxylate (1.2 g, 47.40%) as a yellow solid.

LC-MS: M+H found: 322.

### 37.3. Synthesis of tert-butyl (2R)-2-[([4-[([3-[(3-fluoro-2-methylphenyl)carbamothioyl]-2-oxo-5,6-dihydro-1H-pyridin-4-yl]oxy)methyl]pyridin-3-yl]oxy)methyl]piperidine-1-carboxylate

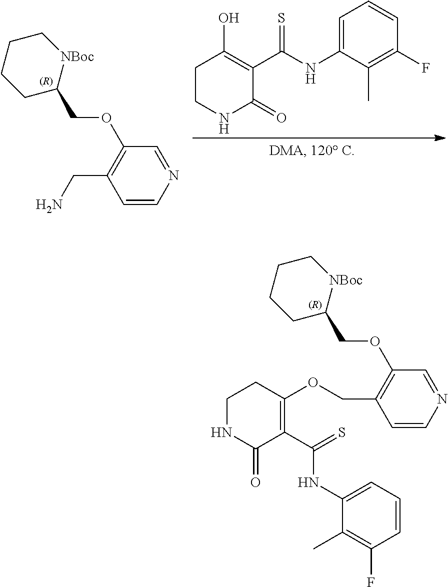

A solution of tert-butyl (2R)-2-([[4-(aminomethyl)pyridin-3-yl]oxy]methyl)piperidine-1-carboxylate (800.00 mg, 2.489 mmol, 1.00 equiv) and N-(3-fluoro-2-methylphenyl)-4-hydroxy-2-oxo-5,6-dihydro-1H-pyridine-3-carbothioamide (558.16 mg, 1.991 mmol, 0.80 equiv) in DMA (5.00 mL) was stirred for 2h at 80 degrees C. The reaction was quenched with water (50 mL), extracted with EA (3×30 mL). The combined organic layers were washed with brine (2×10 mL), dried over anhydrous Na₂SO₄. After filtration, the filtrate was concentrated under reduced pressure. The residue was purified by Prep-TLC (DCM:MeOH=20:1) to afford tert-butyl (2R)-2-[([4-[([3-[(3-fluoro-2-methylphenyl)carbamothioyl]-2-oxo-5,6-dihydro-1H-pyridin-4-yl]oxy)methyl]pyridin-3-yl]oxy)methyl]piperidine-1-carboxylate (580 mg, 32.88%) as a yellow oil.

LC-MS: M+H found: 585.

### 37.4. Synthesis of tert-butyl (2R)-2-[[(4-[3-[(3-fluoro-2-methylphenyl)amino]-4-oxo-1H,5H,6H,7H-pyrrolo[3,2-c]pyridin-2-yl]pyridin-3-yl)oxy]methyl]piperidine-1-carboxylate

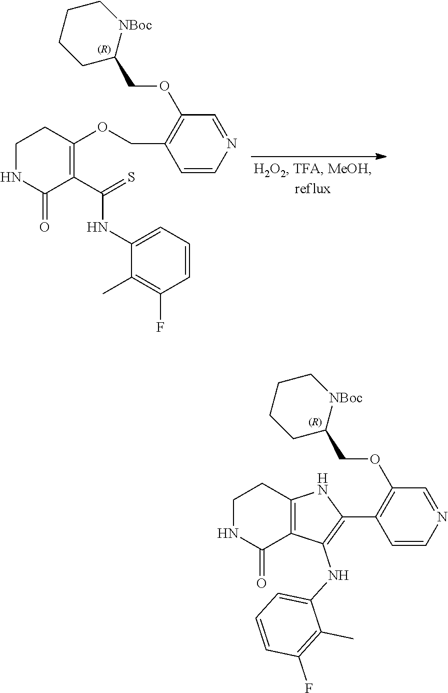

To a stirred solution of tert-butyl (2R)-2-[([4-[([3-[(3-fluoro-2-methylphenyl)carbamothioyl]-2-oxo-5,6-dihydro-1H-pyridin-4-yl]oxy)methyl]pyridin-3-yl]oxy)methyl]piperidine-1-carboxylate (580.00 mg, 0.992 mmol, 1.00 equiv) in MeOH (6.00 mL), H₂O₂ (30%) (57.36 mg, 1.686 mmol, 1.70 equiv) and TFA (113.10 mg, 0.992 mmol, 1 equiv) were added at rt. The resulting mixture was stirred for 2h at 80 degrees C. The mixture was purified by Prep-TLC (DCM:MeOH=20:1) to afford tert-butyl (2R)-2-[[(4-[3-[(3-fluoro-2-methylphenyl)amino]-4-oxo-1H,5H,6H,7H-pyrrolo[3,2-c]pyridin-2-yl]pyridin-3-yl)oxy]methyl]piperidine-1-carboxylate (130 mg, 23.84%) as a yellow solid.

LC-MS: M+H found: 550

### 37.5. Synthesis of 3-[(3-fluoro-2-methylphenyl)amino]-2-[3-[(2R)-piperidin-2-ylmethoxy]pyridin-4-yl]-1H,5H,6H,7H-pyrrolo[3,2-c]pyridin-4-one

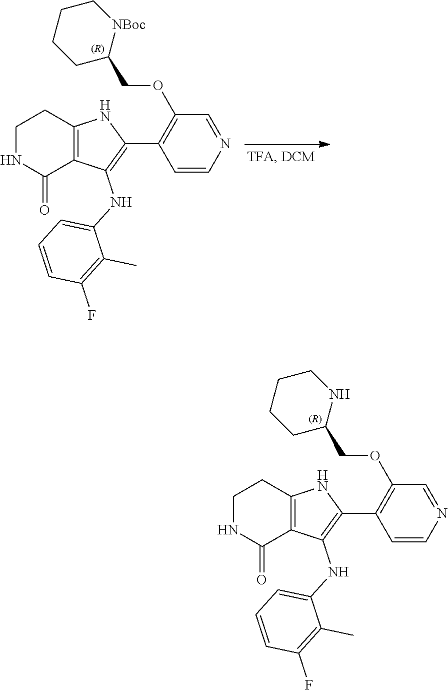

A solution of tert-butyl (2R)-2-[[(4-[3-[(3-fluoro-2-methylphenyl)amino]-4-oxo-1H,5H,6H,7H-pyrrolo[3,2-c]pyridin-2-yl]pyridin-3-yl)oxy]methyl]piperidine-1-carboxylate (240.00 mg) in DCM (6.00 mL), TFA (2.00 mL) was added and stirred for 2h at rt. The resulting mixture was concentrated under reduced pressure to afford 3-[(3-fluoro-2-methylphenyl)amino]-2-[3-[(2R)-piperidin-2-ylmethoxy]pyridin-4-yl]-1H,5H,6H,7H-pyrrolo[3,2-c]pyridin-4-one (180 mg, crude) as a brown semi-solid.

LC-MS: M+H found: 450

### 37.6. Synthesis of 3-[(3-fluoro-2-methylphenyl)amino]-2-(3-[[(2R)-1-(prop-2-enoyl)piperidin-2-yl]methoxy]pyridin-4-yl)-1H,5H,6H,7H-pyrrolo[3,2-c]pyridin-4-one

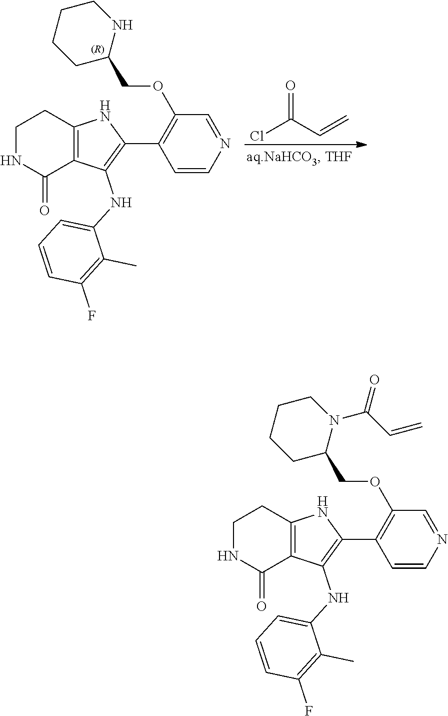

To a solution of 3-[(3-fluoro-2-methylphenyl)amino]-2-[3-[(2R)-piperidin-2-ylmethoxy]pyridin-4-yl]-1H,5H,6H,7H-pyrrolo[3,2-c]pyridin-4-one (70.00 mg, 0.156 mmol, 1.00 equiv) in THF (4.00 mL) was added NaHCO₃ (2.00 mL) at 0 degrees C. The mixture was stirred for 5 min. acryloyl chloride (42.28 mg, 0.467 mmol, 3.00 equiv) was added and the mixture was allowed to warm to RT and stirred for 1 h. The reaction mixture was quenched by water (25 mL) and extracted with EA (3*25 mL). The residue was purified by Prep-TLC (DCM:MeOH 10:1) to afford crude product. The crude product was purified by Prep-HPLC with the following conditions (Column: YMC-Actus Triart C18 ExRS, 30*150 mm, 5 μm; Mobile Phase A: Water (10 MMOL/L NH4HCO3+0.1% NH3·H₂O), Mobile Phase B: ACN; Flow rate: 60 mL/min; Gradient: 28% B to 61% B in 7 min; Wave Length: 254 nm; RT1(min): 6.88;) to afford 3-[(3-fluoro-2-methylphenyl)amino]-2-(3-[[(2R)-1-(prop-2-enoyl)piperidin-2-yl]methoxy]pyridin-4-yl)-1H,5H,6H,7H-pyrrolo[3,2-c]pyridin-4-one (10.1 mg, 12.78%) as a light yellow solid.

LC-MS: M+H found: 504

¹H NMR (400 MHz, DMSO-d6): δ 10.82 (s, 1H), 8.44 (s, 1H), 7.98-7.93 (m, 1H), 7.30 (d, J=12.9 Hz, 2H), 6.99 (s, 1H), 6.75 (d, J=8.4 Hz, 2H), 6.46 (t, J=8.7 Hz, 1H), 6.06 (d, J=8.3 Hz, 2H), 5.72-5.56 (m, 1H), 5.39-5.11 (m, 1H), 4.72 (m, 1H), 4.79-4.65 (m, 2H), 3.44 (dt, J=7.4, 4.3 Hz, 2H), 3.05-2.90 (m, 3H), 2.21 (s, 3H), 1.89-1.78 (m, 1H), 1.78-1.55 (m, 4H), 1.52-1.37 (m, 1H).

### Example 38. 3-[(3-fluoro-2-methylphenyl)amino]-2-[3-[(2S)-piperidin-2-ylmethoxy]pyridin-4-yl]-1H,5H,6H,7H-pyrrolo[3,2-c]pyridin-4-one (compound 469)

### 38.1. Synthesis of tert-butyl tert-butyl (2S)-2-[[(4-cyanopyridin-3-yl)oxy]methyl]piperidine-1-carboxylate

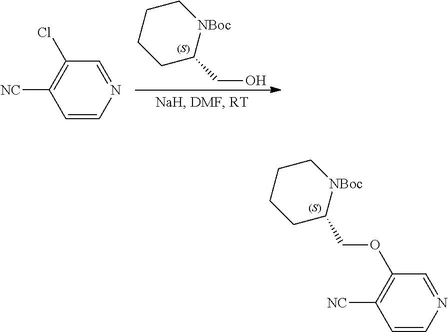

To a solution of 3-chloropyridine-4-carbonitrile (1.23 g, 8.878 mmol, 1.00 equiv) and tert-butyl (2S)-2-(hydroxymethyl)piperidine-1-carboxylate (2.29 g, 10.653 mmol, 1.2 equiv,) in DMF (30.00 mL) were added NaH (426.09 mg, 10.653 mmol, 1.2 equiv, 60%) at 0 degrees C. After stirring for overnight at rt. The aqueous layer was extracted with EA (3×50 mL). The resulting mixture was washed with 2×30 mL of saturated brine. The residue was purified by silica gel column chromatography, eluted with PE:EA (1:1) to afford tert-butyl tert-butyl (2S)-2-[[(4-cyanopyridin-3-yl)oxy]methyl]piperidine-1-carboxylate (2.3 g, 81.63%) as a light orange oil.

LC-MS: M+H found: 318.10

### 38.2. Synthesis of tert-butyl (2S)-2-([[4-(aminomethyl)pyridin-3-yl]oxy]methyl)piperidine-1-carboxylate

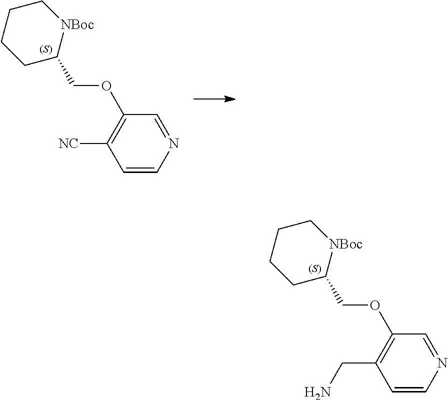

To a solution of Raney Nickel (7.40 g, 126.028 mmol, 10.00 equiv) in NH₃(g) in MeOH (100.00 mL) was added tert-butyl (2S)-2-[[(4-cyanopyridin-3-yl)oxy]methyl]piperidine-1-carboxylate (4.00 g, 12.603 mmol, 1.00 equiv) under nitrogen atmosphere. The mixture was hydrogenated at room temperature for overnight under hydrogen atmosphere using a hydrogen balloon. The precipitated solids were collected by filtration. The residue was purified by silica gel column chromatography, eluted with DCM:MeOH (7:1) to afford tert-butyl (2S)-2-([[4-(aminomethyl)pyridin-3-yl]oxy]methyl)piperidine-1-carboxylate (2.04 g, 50.36%) as a light yellow solid.

LC-MS: M+H found: 322

### 38.3. Synthesis of tert-butyl (2S)-2-[([4-[([3-[(3-fluoro-2-methylphenyl)carbamothioyl]-2-oxo-5,6-dihydro-1H-pyridin-4-yl]oxy)methyl]pyridin-3-yl]oxy)methyl]piperidine-1-carboxylate

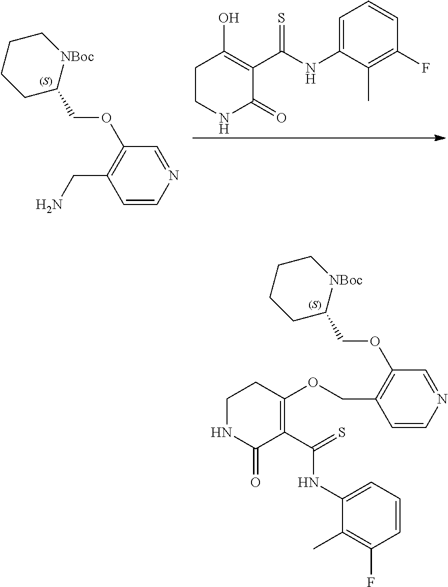

A solution of tert-butyl (2S)-2-([[4-(aminomethyl)pyridin-3-yl]oxy]methyl)piperidine-1-carboxylate (997.56 mg, 3.104 mmol, 1.50 equiv) and N-(3-fluoro-2-methylphenyl)-4-hydroxy-2-oxo-5,6-dihydro-1H-pyridine-3-carbothioamide (580.00 mg, 2.069 mmol, 1.00 equiv) in DMA (6.00 mL) was stirred for 2h at 120 degrees C. The aqueous layer was extracted with EA (3×50 mL). The residue was washed with saturated brine (2×50 mL). The residue was purified by Prep-TLC (DCM:MeOH 20:1) to afford tert-butyl (2S)-2-[([4-[([3-[(3-fluoro-2-methylphenyl)carbamothioyl]-2-oxo-5,6-dihydro-1H-pyridin-4-yl]oxy)methyl]pyridin-3-yl]oxy)methyl]piperidine-1-carboxylate (570 mg, 47.12%) as a yellow solid.

LC-MS: M+H found: 585

### 38.4. Synthesis of tert-butyl (2S)-2-[[(4-[3-[(3-fluoro-2-methylphenyl)amino]-4-oxo-1H,5H,6H,7H-pyrrolo[3,2-c]pyridin-2-yl]pyridin-3-yl)oxy]methyl]piperidine-1-carboxylate

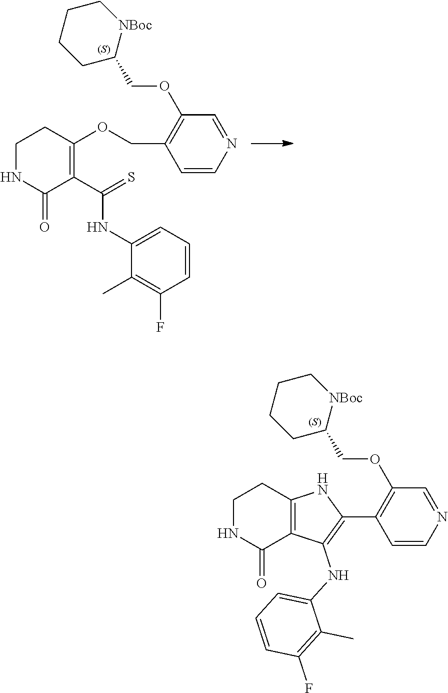

A solution of tert-butyl (2S)-2-[([4-[([3-[(3-fluoro-2-methylphenyl)carbamothioyl]-2-oxo-5,6-dihydro-1H-pyridin-4-yl]oxy)methyl]pyridin-3-yl]oxy)methyl]piperidine-1-carboxylate (500.00 mg, 0.855 mmol, 1.00 equiv) and H₂O₂ (164.83 mg, 1.454 mmol, 1.7 equiv, 30%), TFA (97.50 mg, 0.855 mmol, 1 equiv) in MeOH (6.00 mL) was stirred for 2h at 80 degrees C. The residue was purified by Prep-TLC (DMC:MeOH 15:1) to afford tert-butyl (2S)-2-[[(4-[3-[(3-fluoro-2-methylphenyl)amino]-4-oxo-1H,5H,6H,7H-pyrrolo[3,2-c]pyridin-2-yl]pyridin-3-yl)oxy]methyl]piperidine-1-carboxylate (150 mg, 31.91%) as a orange solid.

LC-MS: M+H found: 550

### 38.5. Synthesis of 3-[(3-fluoro-2-methylphenyl)amino]-2-(3-[[(2S)-1-(prop-2-enoyl)piperidin-2-yl]methoxy]pyridin-4-yl)-1H,5H,6H,7H-pyrrolo[3,2-c]pyridin-4-one

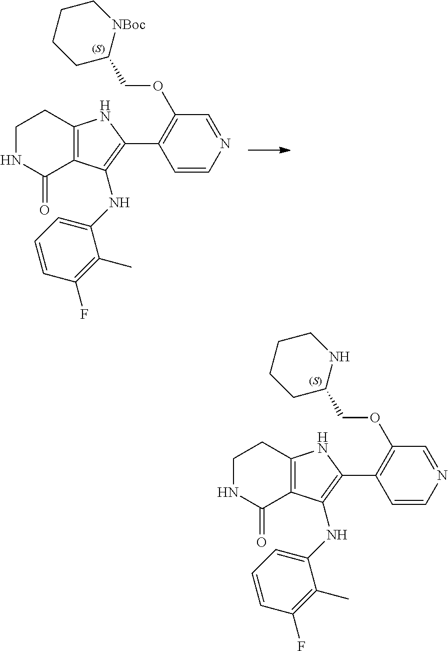

A solution of 3-[(3-fluoro-2-methylphenyl)amino]-2-[3-[(2S)-piperidin-2-ylmethoxy]pyridin-4-yl]-1H,5H,6H,7H-pyrrolo[3,2-c]pyridin-4-one (150.00 mg) in TFA (1.00 mL) and DCM (3.00 mL) was stirred for 2h at rt. The resulting solid was dried under nitrogen atmosphere. This resulted in 3-[(3-fluoro-2-methylphenyl)amino]-2-(3-[[(2S)-1-(prop-2-enoyl)piperidin-2-yl]methoxy]pyridin-4-yl)-1H,5H,6H,7H-pyrrolo[3,2-c]pyridin-4-one (100 mg) as a orange solid.

LC-MS: M+H found: 450.10.

### 38.6. Synthesis of 3-[(3-fluoro-2-methylphenyl)amino]-2-[3-[(2S)-piperidin-2-ylmethoxy]pyridin-4-yl]-1H,5H,6H,7H-pyrrolo[3,2-c]pyridin-4-one

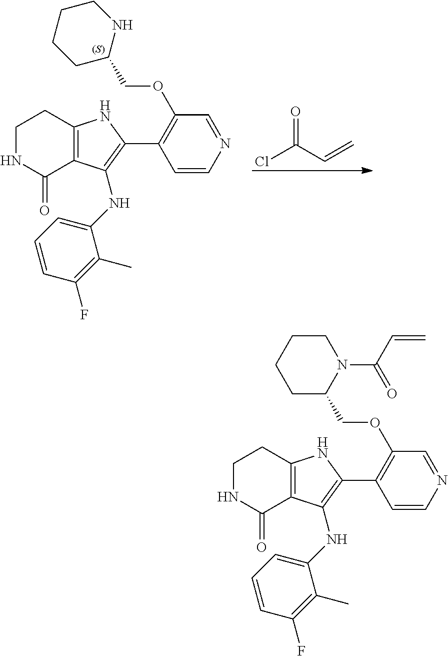

To a solution of tert-butyl (2S)-2-[[(4-[3-[(3-fluoro-2-methylphenyl)amino]-4-oxo-1H,5H,6H,7H-pyrrolo[3,2-c]pyridin-2-yl]pyridin-3-yl)oxy]methyl]piperidine-1-carboxylate (60.00 mg, 0.109 mmol, 1.00 equiv) in THF (4.00 mL) was added NaHCO₃ (2.00 mL, 0.024 mmol, 0.22 equiv) at 0 degrees C. The mixture was stirred for 5 min, acryloyl chloride (29.64 mg, 0.327 mmol, 3.00 equiv) was added and the mixture was allowed to warm to RT and stirred for 1 h. The reaction mixture was quenched by water (25 mL) and extracted with EA (3*25 mL). The residue was purified by Prep-TLC (DCM:MeOH 10:1) to afford crude product. The crude product was purified by Prep-HPLC with the following conditions (Column: YMC-Actus Triart C18 ExRS, 30*150 mm, 5 μm; Mobile Phase A: Water (10 MMOL/L NH4HCO3+0.1% NH3·H2O), Mobile Phase B: ACN; Flow rate: 60 mL/min; Gradient: 28% B to 61% B in 7 min; Wave Length: 254 nm; RT1(min): 6.88;) to afford 3-[(3-fluoro-2-methylphenyl)amino]-2-[3-[(2S)-piperidin-2-ylmethoxy]pyridin-4-yl]-1H,5H,6H,7H-pyrrolo[3,2-c]pyridin-4-one (4.7 mg, 9.58%) as a yellow solid.

LC-MS: (M+H)+ found 504.25

¹H NMR (400 MHz, DMSO-d6): δ 10.76 (s, 1H), 8.43 (s, 1H), 7.95 (d, J=5.0 Hz, 1H), 7.30 (d, J=5.0 Hz, 1H), 7.22 (s, 1H), 6.85 (s, 1H), 6.75 (q, J=10.0, 8.4 Hz, 2H), 6.45 (t, J=8.9 Hz, 1H), 6.08 (d, J=8.2 Hz, 1H), 6.04 (d, J=16.7 Hz, 1H), 5.62 (d, J=10.6 Hz, 1H), 4.25-4.90 (m, 1H), 4.66 (s, 1H), 4.18-4.01 (m, 2H), 3.49-3.42 (m, 3H), 3.05-2.99 (m, 2H), 2.21 (s, 3H), 1.89-1.80 (m, 1H), 1.71-1.61 (m, 4H), 1.48-1.42 (m, 1H).

### Example 39. 3-[(3-chloro-2-methoxyphenyl)amino]-2-(3-[[(2S)-1-(prop-2-enoyl)azetidin-2-yl]methoxy]pyridin-4-yl)-1H,5H,6H,7H-pyrrolo[3,2-c]pyridin-4-one (compound 145b)

### 39.1. Synthesis of tert-butyl (2S)-2-{[(4-cyanopyridin-3-yl)oxy]methyl}azetidine-1-carboxylate

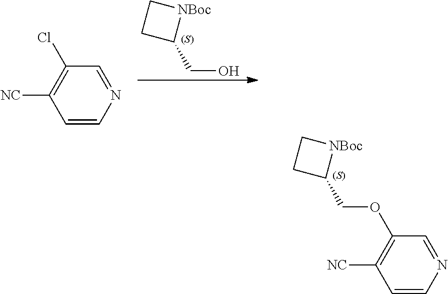

To a stirred solution of tert-butyl (2S)-2-(hydroxymethyl)azetidine-1-carboxylate (2.70 g, 14.436 mmol, 1 equiv) in DMF was added NaH (415.69 mg, 17.323 mmol, 1.2 equiv) in portions at 0 degrees C. under nitrogen atmosphere. The resulting mixture was stirred for 0.5 h at room temperature under nitrogen atmosphere. To the above mixture was added 3-chloropyridine-4-carbonitrile (1.80 g, 12.992 mmol, 0.9 equiv) in portions at 0 degrees C. The resulting mixture was stirred for additional 16 h at room temperature. The reaction was quenched with Water/Ice at room temperature. The resulting mixture was extracted with DCM (4×20 mL). The combined organic layers were washed with brine, dried over anhydrous Na₂SO₄. The residue was purified by silica gel column chromatography, eluted with PE/EtOAc (1:1) to afford tert-butyl (2S)-2-{[(4-cyanopyridin-3-yl)oxy]methyl}azetidine-1-carboxylate (3.3 g, 79.01%) as a yellow oil.

LC-MS: M+H found: 290

### 39.2. Synthesis of tert-butyl (2S)-2-({[4-(aminomethyl)pyridin-3-yl]oxy}methyl)azetidine-1-carboxylate

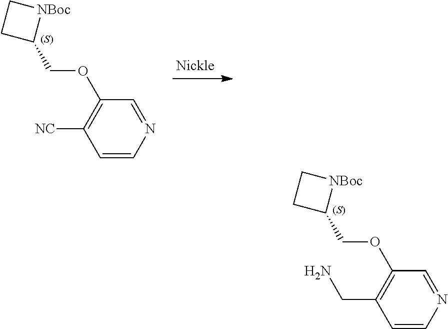

To a stirred solution of tert-butyl (2S)-2-{[(4-cyanopyridin-3-yl)oxy]methyl}azetidine-1-carboxylate (3.3 g, 11.405 mmol, 1.00 equiv) in NH₃(g) in MeOH (50.00 mL) was added Raney Nickel (9.77 g, 114.050 mmol, 10 equiv) at room temperature under hydrogen atmosphere. The resulting mixture was stirred for overnight at room temperature under hydrogen atmosphere. The resulting mixture was filtered, the filter cake was washed with MeOH. The filtrate was concentrated under reduced pressure. The residue was purified by Prep-TLC (DCM/MeOH 10:1) to afford tert-butyl (2S)-2-({[4-(aminomethyl)pyridin-3-yl]oxy}methyl)azetidine-1-carboxylate (2.7 g, 80.69%) as a yellow oil.

LC-MS: M+H found: 294

### 39.3. Synthesis of tert-butyl (2S)-2-[({4-[({3-[(3-chloro-2-methoxyphenyl)carbamothioyl]-2-oxo-5,6-dihydro-1H-pyridin-4-yl}amino)methyl]pyridin-3-yl}oxy)methyl]azetidine-1-carboxylate

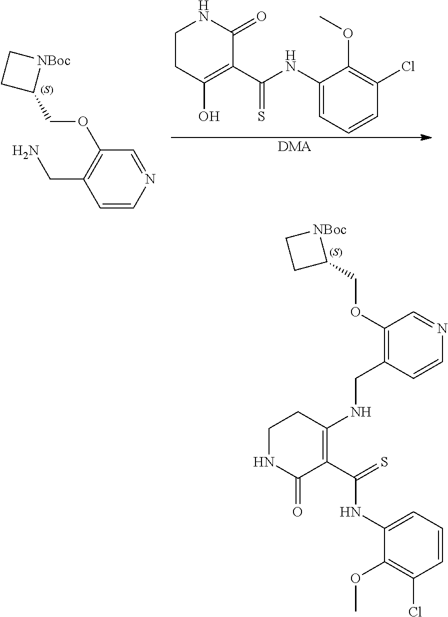

Into a 8 mL round-bottom flask were added tert-butyl (2S)-2-({[4-(aminomethyl)pyridin-3-yl]oxy}methyl)azetidine-1-carboxylate (1013.00 mg, 3.454 mmol, 1.2 equiv) and N-(3-chloro-2-methoxyphenyl)-4-hydroxy-2-oxo-5,6-dihydro-1H-pyridine-3-carbothioamide (900 mg, 2.878 mmol, 1.00 equiv) in DMA (3 mL, 32.266 mmol, 11.21 equiv). The resulting mixture was stirred for 2 h at 120 degrees C. under nitrogen atmosphere. The resulting mixture was extracted with CH2Cl2 and purified by Prep-TLC (DCM/MeOH 10:1) to afford tert-butyl (2S)-2-[({4-[({3-[(3-chloro-2-methoxyphenyl)carbamothioyl]-2-oxo-5,6-dihydro-1H-pyridin-4-yl}amino)methyl]pyridin-3-yl}oxy)methyl]azetidine-1-carboxylate (550 mg, 32.50%) as a yellow solid.

LC-MS: M+H found: 588

### 39.4. Synthesis of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}azetidine-1-carboxylate

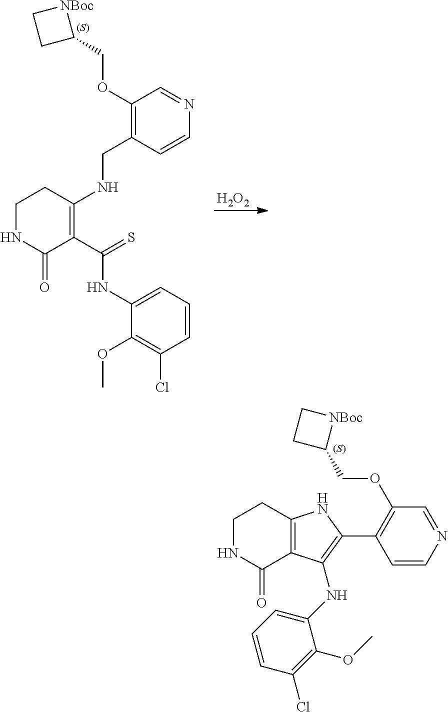

To a stirred solution of tert-butyl (2S)-2-[({4-[({3-[(3-chloro-2-methoxyphenyl)carbamothioyl]-2-oxo-5,6-dihydro-1H-pyridin-4-yl}amino)methyl]pyridin-3-yl}oxy)methyl]azetidine-1-carboxylate (300 mg, 0.510 mmol, 1 equiv) in MeOH was added hydrogen peroxide (35%) (26.03 mg, 0.765 mmol, 1.5 equiv) at room temperature under nitrogen atmosphere. The resulting mixture was stirred for 2 h at 80° C. under nitrogen atmosphere. The resulting mixture was extracted with CH2Cl2 (3×10 mL). The combined organic layers were concentrated under reduced pressure. The residue was purified by Prep-TLC (CH2Cl2/MeOH 10:1) to afford tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}azetidine-1-carboxylate (120 mg, 42.46%) as a yellow solid.

LC-MS: M+H found: 554

### 39.5. Synthesis of 2-{3-[(2S)-azetidin-2-ylmethoxy]pyridin-4-yl}-3-[(3-chloro-2-methoxyphenyl)amino]-1H,5H,6H,7H-pyrrolo[3,2-c]pyridin-4-one

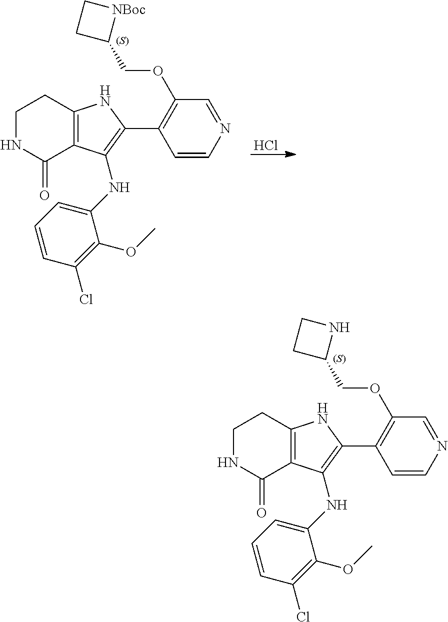

To a stirred solution of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}azetidine-1-carboxylate (110 mg, 0.199 mmol, 1 equiv) in DCM (1 mL) was added TFA (0.50 mL). The resulting mixture was stirred for 2 h at room temperature under air atmosphere. The resulting mixture was concentrated under vacuum. The residue was purified by Prep-TLC (DCM/MeOH 5:1) to afford 2-{3-[(2S)-azetidin-2-ylmethoxy]pyridin-4-yl}-3-[(3-chloro-2-methoxyphenyl)amino]-1H,5H,6H,7H-pyrrolo[3,2-c]pyridin-4-one (85 mg, 94.31%) as a yellow oil.

LC-MS: M+H found: 454.

### 39.6. Synthesis of [(3-chloro-2-methoxyphenyl)amino]-2-(3-[[(2S)-1-(prop-2-enoyl)azetidin-2-yl]methoxy]pyridin-4-yl)-1H,5H,6H,7H-pyrrolo[3,2-c]pyridin-4-one

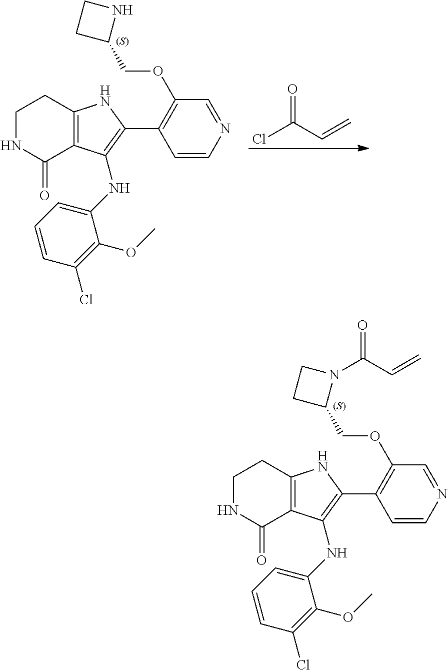

To a stirred mixture of 2-[3-[(2S)-azetidin-2-ylmethoxy]pyridin-4-yl]-3-[(3-chloro-2-methoxyphenyl)amino]-1H,5H,6H,7H-pyrrolo[3,2-c]pyridin-4-one (100.00 mg, 0.220 mmol, 1.00 equiv) in THF (1.00 mL) were added saturated NaHCO₃ (1 mL) and acryloyl chloride (29.91 mg, 0.330 mmol, 1.50 equiv) in portions at RT for 10 min. The resulting mixture was extracted with EA (3×10 ml). The combined organic layers were washed with EA (3×5 ml), dried over anhydrous sodium sulphate. After filtration, the filtrate was concentrated under reduced pressure. The crude product (mg) was purified by Prep-HPLC with the following conditions (Column:Poroshell HPH C18 3.0*50 mm, 2.7 um; Mobile Phase A:Water/6.5 mM NH₄HCO₃(PH=10); Mobile phase B: ACN; Flow rate: 1.2 mL/min; Gradient: 10% B to 95% B in 1.1 min, hold 0.6 min; 254 nm) to afford 3-[(3-chloro-2-methoxyphenyl)amino]-2-(3-[[(2S)-1-(prop-2-enoyl)azetidin-2-yl]methoxy]pyridin-4-yl)-1H,5H,6H,7H-pyrrolo[3,2-c]pyridin-4-one (24.8 mg, 22.16%) as a yellow solid.

LC-MS: M+H found: 508

¹H NMR (400 MHz, DMSO-d6) δ 11.60 (s, 1H), 8.38 (s, 1H), 7.98 (d, J=5.1 Hz, 1H), 7.39 (s, 1H), 7.34 (d, J=5.0 Hz, 1H), 6.78 (s, 1H), 6.71-6.58 (m, 2H), 6.33 (dd, J=16.9, 10.3 Hz, 1H), 6.17 (dd, J=6.8, 2.8 Hz, 2H), 5.68 (s, 1H), 4.84 (d, J=7.4 Hz, 1H), 4.56-4.45 (m, 1H), 4.42 (dd, J=10.7, 2.9 Hz, 1H), 4.15 (s, 2H), 3.91 (s, 3H), 3.44 (td, J=6.9, 2.5 Hz, 2H), 2.94 (s, 2H), 2.52 (m, 1H), 2.15 (m, 1H).

### Example 40. 3-[(3-chloro-2-methoxyphenyl)amino]-2-[3-(2-methanesulfonylethoxy)pyridin-4-yl]-1H,5H,6H,7H-pyrrolo[3,2-c]pyridin-4-one (compound 137)

### 40.1. Synthesis of 3-[2-(trimethylsilyl)ethoxy]pyridine-4-carbonitrile

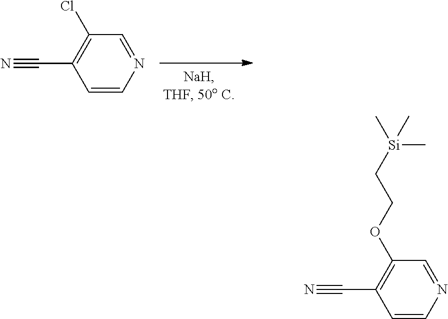

To a solution of 2-(trimethylsilyl)ethanol (6.83 g, 57.741 mmol, 2.00 equiv) in THF (100.00 mL) was added NaH (2.31 g, 57.741 mmol, 2.00 equiv, 60%) at 0 degrees C. The mixture was stirred for 1 h. 3-chloropyridine-4-carbonitrile (4.00 g, 28.870 mmol, 1.00 equiv) was added and the mixture was allowed to warm to 50 degrees C. and stirred for 16h. The reaction mixture was quenched by water (100 mL) and extracted with EA (3*100 mL). The combined organic layers were washed with brine (1×50 mL), dried over anhydrous Na₂SO₄. After filtration, the filtrate was concentrated under reduced pressure. The residue was purified by silica gel column chromatography, eluted with PE:EA (2:1) to afford 3-[2-(trimethylsilyl)ethoxy]pyridine-4-carbonitrile (4.3 g, 67.59%) as a yellow oil.

LC-MS: M+H found: 221

### 40.2. Synthesis of 3-hydroxypyridine-4-carbonitrile

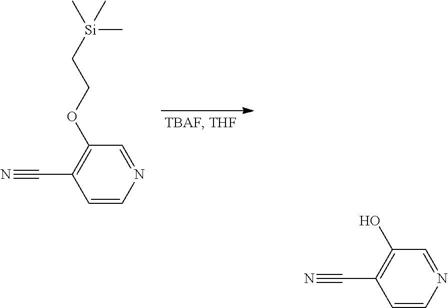

To a stirred solution of 3-[2-(trimethylsilyl)ethoxy]pyridine-4-carbonitrile (4.30 g, 19.515 mmol, 1.00 equiv) in THF (50.00 mL) was added TBAF (10.20 g, 39.029 mmol, 2 equiv) dropwise at 0 degrees C. The resulting mixture was stirred for 2h at rt. The resulting mixture was diluted with H₂O (100 mL). The resulting mixture was extracted with EA (3×100 mL). The combined organic layers were washed with brine (1×50 mL), dried over anhydrous Na₂SO₄. After filtration, the filtrate was concentrated under reduced pressure. The residue was purified by silica gel column chromatography, eluted with DCM:MeOH (10:1) to afford 3-hydroxypyridine-4-carbonitrile (2 g, 85.33%) as a white solid.

LC-MS: M+H found: 121.

### 40.3. Synthesis of 4-(aminomethyl)pyridin-3-ol

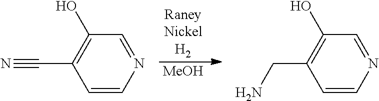 [SMILES: "MeOH" ; N#Cc1ccncc1O ; NCc1ccncc1O]

To a solution of Raney Nickel (1.03 g, 17.484 mmol, 1.00 equiv) in NH3(g) in MeOH (100.00 mL) was added 3-hydroxypyridine-4-carbonitrile (2.10 g, 17.484 mmol, 1.00 equiv) under nitrogen atmosphere. The mixture was hydrogenated at room temperature for 16h under hydrogen atmosphere using a hydrogen balloon, filtered through a Celite pad and concentrated under reduced pressure to afford 4-(aminomethyl)pyridin-3-ol (1.8 g, 82.93%) as a grey solid.

LC-MS: M+H found: 125.

### 40.4. Synthesis of N-(3-chloro-2-methoxyphenyl)-4-[[(3-hydroxypyridin-4-yl)methyl]amino]-2-oxo-5,6-dihydro-1H-pyridine-3-carbothioamide

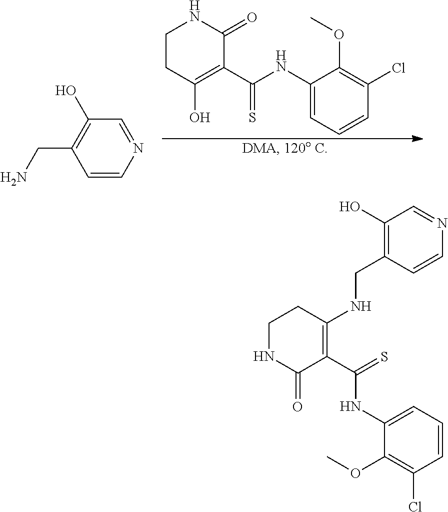

To a stirred solution of N-(3-chloro-2-methoxyphenyl)-4-hydroxy-2-oxo-5,6-dihydro-1H-pyridine-3-carbothioamide (0.80 g, 2.558 mmol, 1.00 equiv) and 4-(aminomethyl)pyridin-3-ol (0.63 g, 5.090 mmol, 1.99 equiv) in DMA (10.00 mL) at rt. The resulting mixture was stirred for 2h at 120 degrees C. The resulting mixture was diluted with H₂O (100 mL). The resulting mixture was extracted with EA (3×100 mL). The combined organic layers were washed with brine (1×50 mL), dried over anhydrous Na₂SO₄. After filtration, the filtrate was concentrated under reduced pressure. The residue was purified by Prep-TLC (DCM:MeOH=15:1) to afford N-(3-chloro-2-methoxyphenyl)-4-[[(3-hydroxypyridin-4-yl)methyl]amino]-2-oxo-5,6-dihydro-1H-pyridine-3-carbothioamide (600 mg, 56.00%) as a brown oil.

LC-MS: M+H found: 419

### 40.5. Synthesis of 3-[(3-chloro-2-methoxyphenyl)amino]-2-(3-hydroxypyridin-4-yl)-1H,5H,6H,7H-pyrrolo[3,2-c]pyridin-4-one

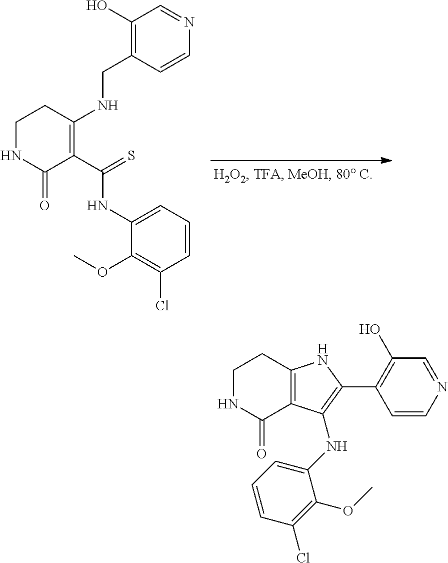 [SMILES: COc1c(Cl)cccc1NC(=S)C1=C(NCc2ccncc2O)CCNC1=O ; COc1c(Cl)cccc1Nc1c(-c2ccncc2O)[nH]c2c1C(=O)NCC2]

To a stirred solution of N-(3-chloro-2-methoxyphenyl)-4-[[(3-hydroxypyridin-4-yl)methyl]amino]-2-oxo-5,6-dihydro-1H-pyridine-3-carbothioamide (600.00 mg, 1.432 mmol, 1.00 equiv) in MeOH (8.00 mL) ware added H₂O₂ (82.82 mg, 2.435 mmol, 1.7 equiv) and TFA (163.32 mg, 1.432 mmol, 1 equiv) dropwise at rt. The resulting mixture was stirred for 2h at 80 degrees C. The resulting mixture was diluted with H₂O (100 mL). The resulting mixture was extracted with EA (3×100 mL). The combined organic layers were washed with brine (1×50 mL), dried over anhydrous Na₂SO₄. After filtration, the filtrate was concentrated under reduced pressure. The residue was purified by Prep-TLC (DCM:MeOH=10:1) to afford 3-[(3-chloro-2-methoxyphenyl)amino]-2-(3-hydroxypyridin-4-yl)-1H,5H,6H,7H-pyrrolo[3,2-c]pyridin-4-one (300 mg, 54.43%) as a brown solid.

LC-MS: M+H found: 385

### 40.6. Synthesis of 3-[(3-chloro-2-methoxyphenyl)amino]-2-[3-(2-methanesulfonylethoxy)pyridin-4-yl]-1H,5H,6H,7H-pyrrolo[3,2-c]pyridin-4-one

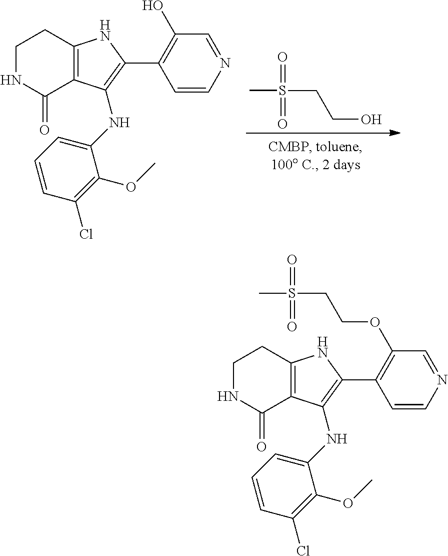

To a stirred solution of 3-[(3-chloro-2-methoxyphenyl)amino]-2-(3-hydroxypyridin-4-yl)-1H,5H,6H,7H-pyrrolo[3,2-c]pyridin-4-one (100.00 mg, 0.260 mmol, 1.00 equiv) and 2-methanesulfonylethanol (64.52 mg, 0.520 mmol, 2.00 equiv) in Toluene (2.00 mL) was added 2-(tributyl-l\hat{ }[5]-phosphanylidene)acetonitrile (125.44 mg, 0.520 mmol, 2 equiv) dropwise at rt under N₂ atmosphere. The resulting mixture was stirred for two days at 100 degrees C. under N₂ atmosphere. The resulting mixture was diluted with H₂O (30 mL). The resulting mixture was extracted with EA (3×50 mL). The combined organic layers were washed with brine (1×30 mL), dried over anhydrous Na₂SO₄. After filtration, the filtrate was concentrated under reduced pressure. The residue was purified by Prep-TLC (DCM:MeOH=5:1) to afford crude product. The crude product (30 mg) was purified by Prep-HPLC with the following conditions (Column: YMC-Actus Triart C18, 30*150 mm, 5 μm; Mobile Phase A: Water (10 MMOL/L NH₄HCO_{3+0.1}% NH3·H2O), Mobile Phase B: ACN; Flow rate: 60 mL/min; Gradient: 25% B to 35% B in 8 min; Wave Length: 254 nm; RT1(min): 8.05;) to afford 3-[(3-chloro-2-methoxyphenyl)amino]-2-[3-(2-methanesulfonylethoxy)pyridin-4-yl]-1H,5H,6H,7H-pyrrolo[3,2-c]pyridin-4-one (8.4 mg, 6.54%) as a yellow solid.

LC-MS: M+H found: 491

¹H NMR (400 MHz, DMSO-d6): δ 14.06 (s, 1H), 7.68 (s, 2H), 7.49 (d, J=6.4 Hz, 1H), 7.26-7.07 (m, 2H), 6.96-6.70 (m, 2H), 6.19 (dd, J=7.3, 2.4 Hz, 1H), 4.55 (t, J=7.1 Hz, 2H), 3.92 (s, 3H), 3.85 (t, J=6.9 Hz, 2H), 3.39 (t, J=6.9 Hz, 2H), 3.02 (s, 3H), 2.91 (t, J=6.9 Hz, 2H).

### Example 41. 3-[(3-chloro-2-methoxyphenyl)amino]-2-[3-(furan-2-ylmethoxy)pyridin-4-yl]-1H,5H,6H,7H-pyrrolo[3,2-c]pyridin-4-one (compound 471)

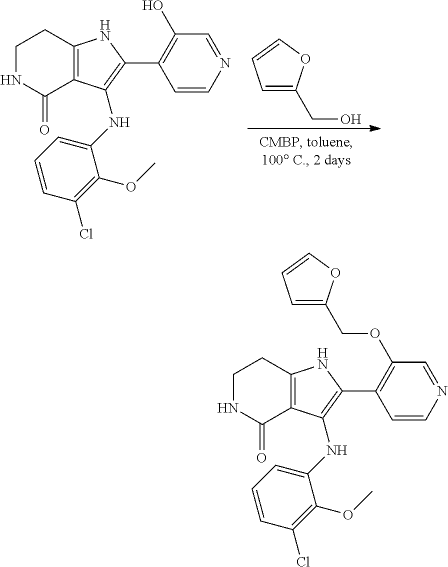

To a stirred solution of 3-[(3-chloro-2-methoxyphenyl)amino]-2-(3-hydroxypyridin-4-yl)-1H,5H,6H,7H-pyrrolo[3,2-c]pyridin-4-one (100.00 mg, 0.260 mmol, 1.00 equiv) and furfuryl alcohol (50.99 mg, 0.520 mmol, 2.00 equiv) in Toluene (2.00 mL, 18.798 mmol, 72.34 equiv) was added 2-(tributyl-l\hat{ }[5]-phosphanylidene)acetonitrile (125.44 mg, 0.520 mmol, 2 equiv) dropwise at rt under N₂ atmosphere. The resulting mixture was stirred for two days at 100 degrees C. under N₂ atmosphere. The resulting mixture was diluted with H₂O (30 mL). The resulting mixture was extracted with EA (3×50 mL). The combined organic layers were washed with brine (1×30 mL), dried over anhydrous Na₂SO₄. After filtration, the filtrate was concentrated under reduced pressure. The residue was purified by Prep-TLC (DCM:MeOH=10:1) to afford crude product. The crude product (60 mg) was purified by Prep-HPLC with the following conditions (Column: XBridge Shield RP18 OBD Column, 30*150 mm, 5 μm; Mobile Phase A: Water (10 MMOL/L NH4HCO3+0.1% NH₃·H2O), Mobile Phase B: ACN; Flow rate: 60 mL/min; Gradient: 31% B to 41% B in 8 min; Wave Length: 254 nm; RT1(min): 7.13;) to afford 3-[(3-chloro-2-methoxyphenyl)amino]-2-[3-(furan-2-ylmethoxy)pyridin-4-yl]-1H,5H,6H,7H-pyrrolo[3,2-c]pyridin-4-one (14.3 mg, 11.60%) as a yellow solid. LC-MS: M+H found: 465

¹H NMR (400 MHz, DMSO-d6): δ 14.04 (s, 1H), 7.71 (d, J=1.8 Hz, 1H), 7.65 (s, 1H), 7.56 (d, J=1.9 Hz, 1H), 7.51-7.41 (m, 1H), 7.21-7.09 (m, 2H), 6.86-6.73 (m, 2H), 6.68 (d, J=3.3 Hz, 1H), 6.53-6.46 (m, 1H), 6.19 (dd, J=7.6, 2.1 Hz, 1H), 5.39 (s, 2H), 3.92 (s, 3H), 3.48-3.39 (t, 2H), 2.90 (t, J=6.8 Hz, 2H).

### Example 42. 3-[(3-fluoro-2-methoxyphenyl)amino]-2-[3-[(1-methylpyrazol-4-yl)oxy]pyridin-4-yl]-1H,5H,6H,7H-pyrrolo[3,2-c]pyridin-4-one (compound 484)

### 42.1. Synthesis of 2-[(3-bromopyridin-4-yl)methyl]isoindole-1,3-dione

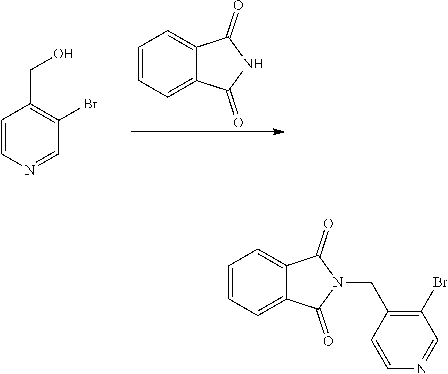 [SMILES: OCc1ccncc1Br ; O=C1NC(=O)c2ccccc21 ; O=C1c2ccccc2C(=O)N1Cc1ccncc1Br]

To a stirred solution of (3-bromopyridin-4-yl)methanol (2000 mg, 10.637 mmol, 1.00 equiv) and phthalimide (2347.57 mg, 15.956 mmol, 1.5 equiv) in THF (15 mL) were added PPh₃ (6974.79 mg, 26.593 mmol, 2.5 equiv) and DIAD (3226.31 mg, 15.955 mmol, 1.50 equiv) dropwise at rt under N₂ atmosphere for 5h. The reaction was quenched by the addition of H₂O (15 ml) at 0° C. The resulting mixture was extracted with EA (3×50 ml). The combined organic layers were washed with NaCl (3×2 30 ml), dried over anhydrous Na₂SO₄. After filtration, the filtrate was concentrated under reduced pressure. The resulting mixture was concentrated under reduced pressure. The residue was purified by Prep-TLC (DCM:MeOH=18:1) to afford 2-[(3-bromopyridin-4-yl)methyl]isoindole-1,3-dione (1.5 g, 84.47%) as off white solid.

LC-MS: (M+H)+ found: 318.90.

### 42.2. Synthesis of 1-(3-iodopyridin-4-yl) methanamine

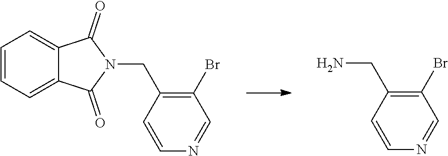 [SMILES: O=C1c2ccccc2C(=O)N1Cc1ccncc1Br ; NCc1ccncc1Br]

To a stirred solution of 2-[(3-iodopyridin-4-yl) methyl]isoindole-1,3-dione (1500 mg, 4.119 mmol, 1.00 equiv) in CH₃₀H (15 mL) was added CH₃₀Na (778.88 mg, 14.416 mmol, 3.5 equiv) in portions at 50° C. under N₂ atmosphere. The resulting mixture was extracted with EA (3×100 ml). The combined organic layers were washed with NaCl (3×1 20 ml), dried over anhydrous Na₂SO₄. After filtration, The resulting mixture was concentrated under reduced pressure. The residue was purified by Prep-TLC (DCM:MeOH=10:1) to afford 1-(3-iodopyridin-4-yl) methanamine (500 mg, 61.86%) as a white solid.

LC-MS: (M+H)+ found: 187.62.

### 42.3. Synthesis of 4-{[(3-bromopyridin-4-yl)methyl]amino}-N-(3-fluoro-2-methoxyphenyl)-2-oxo-5,6-dihydro-1H-pyridine-3-carbothioamide

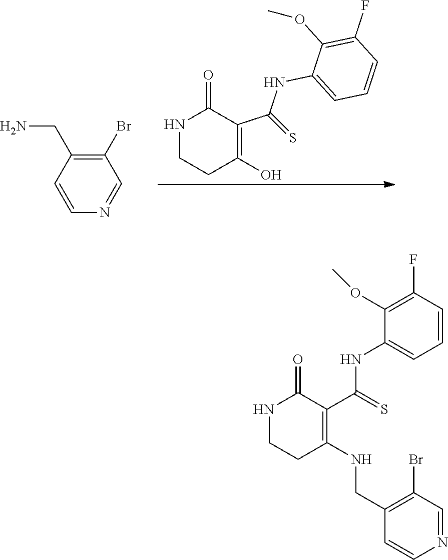 [SMILES: NCc1ccncc1Br ; COc1c(F)cccc1NC(=S)C1=C(O)CCNC1=O ; COc1c(F)cccc1NC(=S)C1=C(NCc2ccncc2Br)CCNC1=O]

To a stirred solution of N-(3-fluoro-2-methoxyphenyl)-4-hydroxy-2-oxo-5,6-dihydro-1H-pyridine-3-carbothioamide (400 mg, 1.350 mmol, 1.00 equiv) and 1-(3-bromopyridin-4-yl)methanamine (302.98 mg, 1.620 mmol, 1.20 equiv) in DMA (2 mL) at 110° C. under N₂ atmosphere. The resulting mixture was extracted with EA (3×20 ml). The combined organic layers were washed with NaCl (3×2 30 ml), dried over anhydrous Na₂SO₄. After filtration, the filtrate was concentrated under reduced pressure. The residue was purified by Prep-TLC (DCM:MeOH=15:1) to afford 4-{[(3-bromopyridin-4-yl)methyl]amino}-N-(3-fluoro-2-methoxyphenyl)-2-oxo-5,6-dihydro-1H-pyridine-3-carbothioamide (200 mg, 70.84%) as a brown solid.

LC-MS: (M+H)+ found: 467.20.

### 42.4. Synthesis of 2-(3-bromopyridin-4-yl)-3-[(3-fluoro-2-methoxyphenyl)amino]-1H,5H,6H,7H-pyrrolo[3,2-c]pyridin-4-one

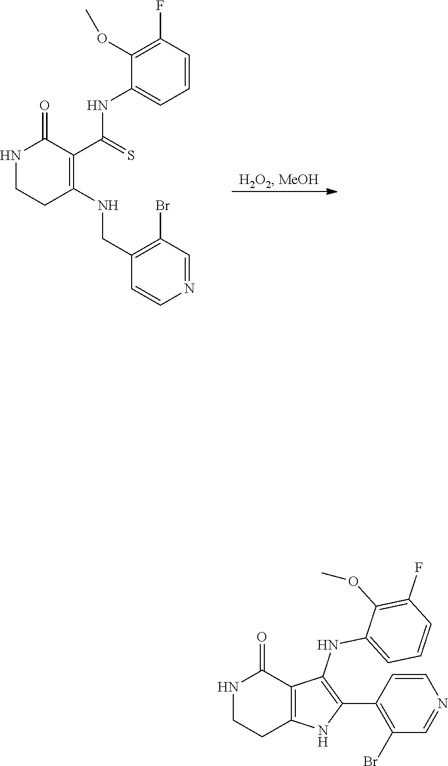

To a stirred solution of 4-{[(3-bromopyridin-4-yl)methyl]amino}-N-(3-fluoro-2-methoxyphenyl)-2-oxo-5,6-dihydro-1H-pyridine-3-carbothioamide (500 mg, 1.074 mmol, 1.00 equiv) in methanol (8 mL, 1.074 mmol, 1.00 equiv) was added H₂O₂ (7.31 mg, 0.215 mmol, 0.2 equiv) dropwise at 50° C. under N₂ atmosphere for 8h. The residue was purified by Prep-TLC (DCM:MeOH=15:1) to afford 2-(3-bromopyridin-4-yl)-3-[(3-fluoro-2-methoxyphenyl)amino]-1H,5H,6H,7H-pyrrolo[3,2-c]pyridin-4-one (150 mg, 50.57%) as off white solid.

LC-MS: (M+H)+ found: 432.95.

### 42.5. Synthesis of 3-[(3-fluoro-2-methoxyphenyl)amino]-2-[3-[(1-methylpyrazol-4-yl)oxy]pyridin-4-yl]-1H,5H,6H,7H-pyrrolo[3,2-c]pyridin-4-one

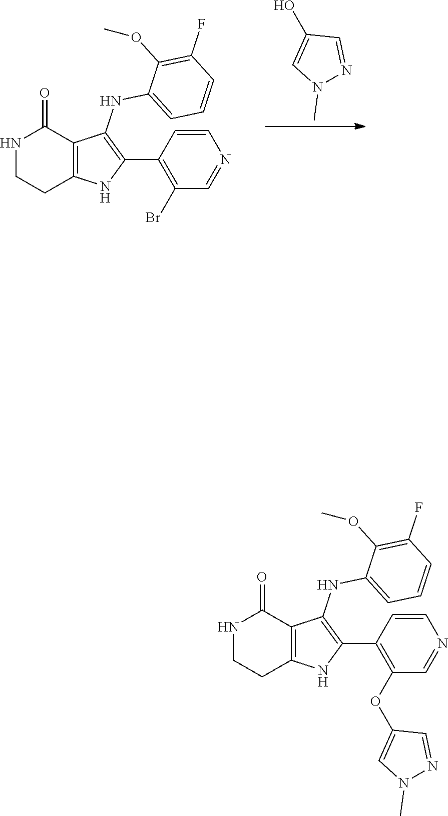

To a stirred solution of 2-(3-bromopyridin-4-yl)-3-[(3-fluoro-2-methoxyphenyl)amino]-1H,5H,6H,7H-pyrrolo[3,2-c]pyridin-4-one (100.00 mg, 0.23 mmol, 1.00 equiv) and 1-methylpyrazol-4-ol (25.0 mg, 0.25 mmol, 1.10 equiv) in DMF (3.5 mL) were added 2,2,6,6-tetramethylheptane-3,5-dione (8.5 mg, 0.005 mmol, 0.20 equiv), CuI (8.8 mg, 0.005 mmol, 0.20 equiv) and Cs₂CO₃ (377.7 mg, 0.116 mmol, 5.00 equiv) in portions at 80° C. under N₂ atmosphere for 2h. The resulting mixture was extracted with EA (3×10 ml). The combined organic layers were washed with NaCl (3×2 5 ml), dried over anhydrous Na₂SO₄. After filtration, the filtrate was concentrated under reduced pressure. The crude product (mg) was purified by Prep-HPLC with the following conditions (Column: YMC-Actus Triart C18, 30*150 mm, 5 μm; Mobile Phase A: Water (10 mmol/L NH₄HCO_{3+0.1}% NH3·H2O), Mobile Phase B: ACN; Flow rate: 60 mL/min; Gradient: 20% B to 45% B in 10 min, 45% B; Wave Length: 254/220 nm; RT1(min): 9.82; Number Of Runs: 0) to afford 3-[(3-fluoro-2-methoxyphenyl)amino]-2-[3-[(1-methylpyrazol-4-yl)oxy]pyridin-4-yl]-1H,5H,6H,7H-pyrrolo[3,2-c]pyridin-4-one (8.6 mg, 97.80%) as a white solid.

LC-MS: (M+H)+ found: 449.1.

¹H NMR (400 MHz, DMSO-d6) δ 11.41 (s, 1H), 8.67-7.94 (m, 2H), 7.64 (s, 1H), 7.53 (d, J=0.8 Hz, 1H), 7.41 (d, J=5.1 Hz, 1H), 7.14 (s, 2H), 7.11 (d, J=1.0 Hz, 1H), 6.62 (d, J=6.2 Hz, 1H), 6.50 (s, 1H), 6.21-5.89 (m, 1H), 3.79 (d, J=1.5 Hz, 6H), 3.41 (dt, J=6.7, 3.4 Hz, 2H), 2.86 (t, J=6.8 Hz, 2H).

### Example 43. 3-[(3-chloro-2-methoxyphenyl)amino]-2-(3-{[(3R)-4-(prop-2-enoyl)morpholin-3-yl]methoxy}pyridin-4-yl)-1H,5H,6H,7H-pyrrolo[3,2-c]pyridin-4-one (compound 522)

### 43.1. Synthesis of tert-butyl (3R)-3-{[(4-cyanopyridin-3-yl)oxy]methyl}morpholine-4-carboxylate

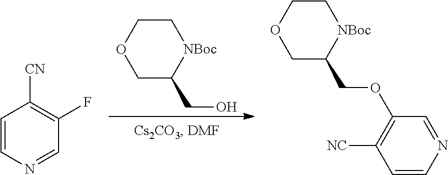

To a stirred solution of tert-butyl (3S)-3-(hydroxymethyl)morpholine-4-carboxylate (3.56 g, 16.38 mmol, 1.00 equiv) and 3-fluoropyridine-4-carbonitrile (2.00 g, 16.38 mmol, 1.00 equiv) in DMF (10 mL) was added Cs₂CO₃ (16.06 g, 49.29 mmol, 3.0 equiv) in portions at room temperature under argon atmosphere. The resulting mixture was stirred for overnight at 60 degrees C. under argon atmosphere. The mixture was allowed to cool down to room temperature. The resulting mixture was extracted with EtOAc (3×100 mL). The combined organic layers were washed with water (3×100 mL), dried over anhydrous Na₂SO₄. After filtration, the filtrate was concentrated under reduced pressure. The residue was purified by silica gel column chromatography, eluted with PE/EtOAc (1:1) to afford tert-butyl (3R)-3-{[(4-cyanopyridin-3-yl)oxy]methyl}morpholine-4-carboxylate (4.2 g, 80.29%) as a yellow solid.

LC-MS: (M+H)⁺ found: 320.05

### 43.2. Synthesis of tert-butyl (3R)-3-({[4-(aminomethyl)pyridin-3-yl]oxy}methyl)morpholine-4-carboxylate

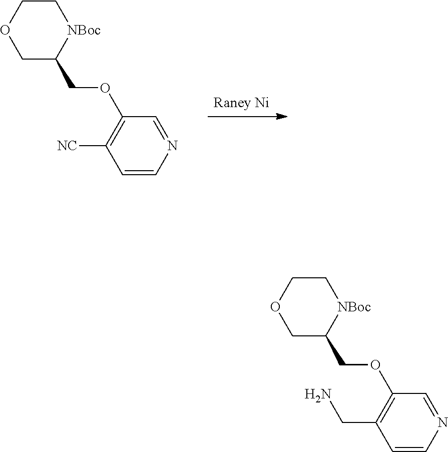 [SMILES: CC(C)(C)OC(=O)N1CCOC[C@@H]1COc1cnccc1C#N ; "Raney Ni" ; CC(C)(C)OC(=O)N1CCOC[C@@H]1COc1cnccc1CN]

To a stirred mixture of tert-butyl (3R)-3-{[(4-cyanopyridin-3-yl)oxy]methyl}morpholine-4-carboxylate (4.20 g, 13.15 mmol, 1.00 equiv) and Ammonia (7.0 M Solution in MeOH, mL, 140.00 mmol) in MeOH (40 mL) and Raney Ni (2.25 g, 54 w/w %) was stirred for overnight at room temperature under hydrogen atmosphere. The resulting mixture was filtered, the filter cake was washed with methanol (3×30 mL). The filtrate was concentrated under reduced pressure. The residue was purified by silica gel column chromatography, eluted with CH2Cl2/MeOH (10:1) to afford tert-butyl (3R)-3-({[4-(aminomethyl)pyridin-3-yl]oxy}methyl)morpholine-4-carboxylate (4.20 g, 98.75%) as a yellow oil.

LC-MS: (M+H)⁺ found 324.05.

### 43.3. Synthesis of tert-butyl (3R)-3-({[4-({[1-(tert-butoxycarbonyl)-3-[(3-chloro-2-methoxyphenyl)carbamothioyl]-2-oxo-5,6-dihydropyridin-4-yl]amino}methyl)pyridin-3-yl]oxy}methyl)morpholine-4-carboxylate

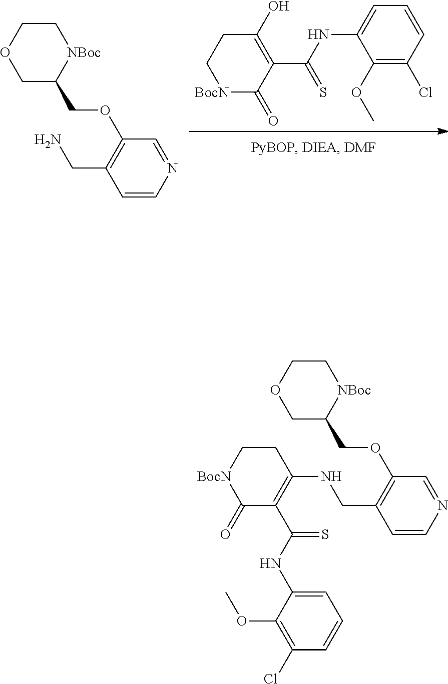

To a stirred mixture of tert-butyl 3-[(3-chloro-2-methoxyphenyl)carbamothioyl]-4-hydroxy-2-oxo-5,6-dihydropyridine-1-carboxylate (5.36 g, 12.99 mmol, 1.00 equiv) and tert-butyl (3R)-3-({[4-(aminomethyl)pyridin-3-yl]oxy}methyl)morpholine-4-carboxylate (4.20 g, 12.99 mmol, 1.00 equiv) in DMF (40 mL) was added DIEA (5.04 g, 38.96 mmol, 3.00 equiv) and PyBOP (10.14 g, 19.48 mmol, 1.50 equiv) at room temperature under argon atmosphere. The resulting mixture was stirred for overnight at room temperature under argon atmosphere. The resulting mixture was diluted with water (100 mL). The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EtOAc (1:2) to afford tert-butyl (3R)-3-({[4-({[1-(tert-butoxycarbonyl)-3-[(3-chloro-2-methoxyphenyl)carbamothioyl]-2-oxo-5,6-dihydropyridin-4-yl]amino}methyl)pyridin-3-yl]oxy}methyl)morpholine-4-carboxylate (6.50 g, 69.68%) as an orange solid.

LC-MS: (M+H)⁺ found 718.0.

### 43.4. Synthesis of tert-butyl (3R)-3-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]morpholine-4-carboxylate

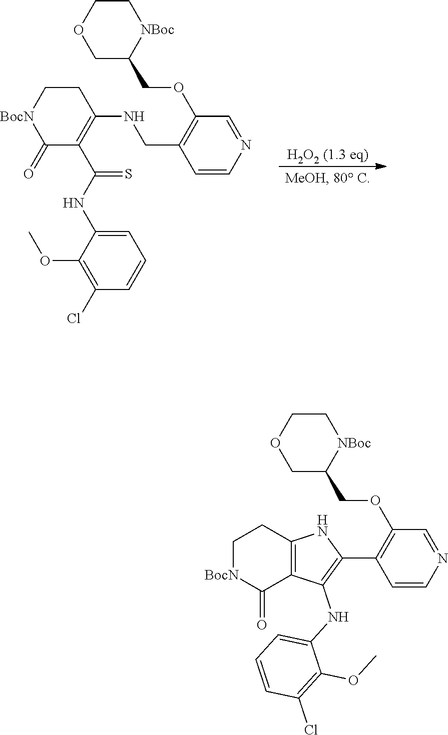 [SMILES: COc1c(Cl)cccc1NC(=S)C1=C(NCc2ccncc2OC[C@H]2COCCN2C(=O)OC(C)(C)C)CCN(C(=O)OC(C)(C)C)C1=O ; "H2O2 (1.3 eq)" ; "MeOH, 80° C." ; COc1c(Cl)cccc1Nc1c(-c2ccncc2OC[C@H]2COCCN2C(=O)OC(C)(C)C)[nH]c2c1C(=O)N(C(=O)OC(C)(C)C)CC2]

To a stirred mixture of tert-butyl (3R)-3-({[4-({[1-(tert-butoxycarbonyl)-3-[(3-chloro-2-methoxyphenyl)carbamothioyl]-2-oxo-5,6-dihydropyridin-4-yl]amino}methyl)pyridin-3-yl]oxy}methyl)morpholine-4-carboxylate (500 mg, 0.70 mmol, 1.00 equiv) in methanol (5 mL) was added hydrogen peroxide (30 w/w %, 103 mg, 0.91 mmol, 1.30 equiv) at room temperature under argon atmosphere. The resulting mixture was stirred for 4 h at 80 degrees C. under argon atmosphere. The mixture was allowed to cool down to room temperature. The reaction was quenched by the addition of sat. Na₂SO₃ (aq.) (0.1 mL) at room temperature. The resulting mixture was concentrated under reduced pressure. The residue was purified by Prep-TLC (CH2Cl2/MeOH 10:1) to afford tert-butyl (3R)-3-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]morpholine-4-carboxylate (200 mg, 41.99%) as a yellow solid.

LC-MS: (M+H)⁺ found: 684.1.

### 43.5. Synthesis of 3-[(3-chloro-2-methoxyphenyl)amino]-2-{3-[(3R)-morpholin-3-ylmethoxy]pyridin-4-yl}-1H,5H,6H,7H-pyrrolo[3,2-c]pyridin-4-one

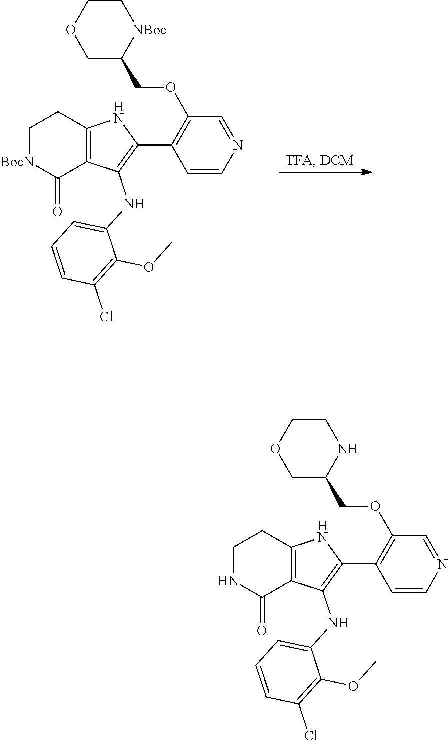

To a stirred mixture of tert-butyl (3R)-3-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]morpholine-4-carboxylate (140 mg, 0.20 mmol, 1.00 equiv) in DCM (5 mL) was added TFA (1 mL) at 0 degree C. under argon atmosphere. The resulting mixture was stirred for 12 h at room temperature under argon atmosphere. The resulting mixture was concentrated under reduced pressure. This resulted in 3-[(3-chloro-2-methoxyphenyl)amino]-2-{3-[(3R)-morpholin-3-ylmethoxy]pyridin-4-yl}-1H,5H,6H,7H-pyrrolo[3,2-c]pyridin-4-one (120 mg, crude) as a yellow solid. The crude product was used in the next step directly without further purification.

LC-MS: (M+H)⁺ found: 484.05.

### 43.6. Synthesis of 3-[(3-chloro-2-methoxyphenyl)amino]-2-(3-{[(3R)-4-(prop-2-enoyl)morpholin-3-yl]methoxy}pyridin-4-yl)-1H,5H,6H,7H-pyrrolo[3,2-c]pyridin-4-one

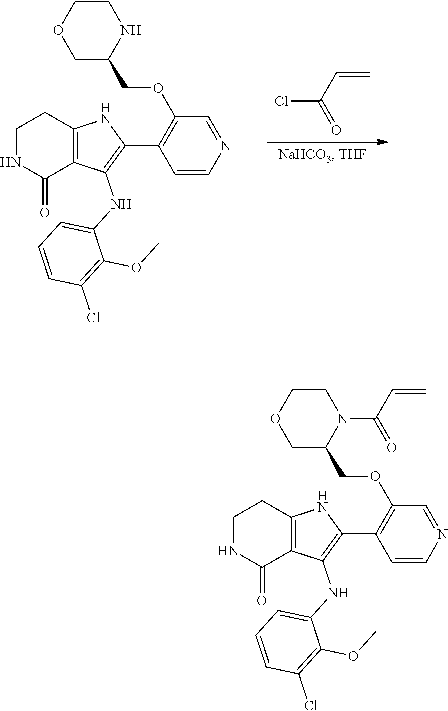

To a stirred mixture of 3-[(3-chloro-2-methoxyphenyl)amino]-2-{3-[(3R)-morpholin-3-ylmethoxy]pyridin-4-yl}-1H,5H,6H,7H-pyrrolo[3,2-c]pyridin-4-one (110 mg, 0.23 mmol, 1.00 equiv) in CH2Cl2 (4 mL) was added triethylamine (93 mg, 0.92 mmol, 5.00 equiv) at 0 degree C. under argon atmosphere at −30 degrees C. To the above mixture was added acryloyl chloride (14.98 mg, 0.17 mmol, 0.90 equiv) in portions at −30 degrees C. The resulting mixture was stirred for additional 1 h at room temperature. The crude product (100 mg) was purified by Prep-HPLC with the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 38% B to 50% B in 10 min, 50% B; Wave Length: 220/254 nm; RT1(min): 7.43; Number Of Runs: 0) to afford 3-[(3-chloro-2-methoxyphenyl)amino]-2-(3-{[(3R)-4-(prop-2-enoyl)morpholin-3-yl]methoxy}pyridin-4-yl)-1H,5H,6H,7H-pyrrolo[3,2-c]pyridin-4-one (19.8 mg, 16.21%) as a white solid.

LC-MS: (M+H)⁺ found: 537.95.

¹H NMR (300 MHz, DMSO-d6) δ 10.93 (s, 1H), 8.47 (s, 1H), 8.14-7.94 (m, 1H), 7.60-7.43 (m, 1H), 7.42-7.25 (m, 1H), 7.17 (t, 1H), 6.96-6.79 (m, 1H), 6.76-6.58 (m, 2H), 6.26-5.96 (m, 2H), 5.83-5.47 (m, 1H), 5.06-4.66 (m, 1H), 4.54-4.14 (m, 2H), 4.11-3.79 (m, 6H), 3.72-3.37 (m, 5H), 3.07-2.78 (m, 2H).

### Example 44. 2-(3-{[(2S)-4-acetylmorpholin-2-yl]methoxy}pyridin-4-yl)-3-[(3-chloro-2-methoxyphenyl)amino]-1H,5H,6H,7H-pyrrolo[3,2-c]pyridin-4-one (compound 195)

### 44.1. Synthesis of tert-butyl (2S)-2-{[(4-cyanopyridin-3-yl)oxy]methyl}morpholine-4-carboxylate

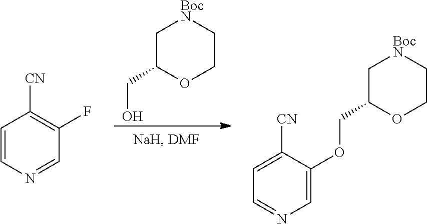

To a stirred mixture of tert-butyl (2S)-2-(hydroxymethyl)morpholine-4-carboxylate (1.96 g, 9.01 mmol, 1.10 equiv) in DMF (20.00 mL) was added NaH (0.49 g, 12.29 mmol, 1.50 equiv, 60% in oil) in portions at 0 degrees C. under argon atmosphere. The resulting mixture was stirred for 30 min at 0 degrees C. under argon atmosphere. To the above mixture was added 3-fluoropyridine-4-carbonitrile (1.00 g, 8.19 mmol, 1.00 equiv) in portions over 5 min at 0 degrees C. The resulting mixture was stirred for additional 2 h at room temperature. The reaction was monitored by LCMS. Desired product could be detected by LCMS. The reaction was quenched with Water at 0 degrees C. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EA (4:1) to afford tert-butyl (2S)-2-{[(4-cyanopyridin-3-yl)oxy]methyl}morpholine-4-carboxylate (1.20 g, 45.42%) as a colorless oil.

LC-MS: M+Na found: 341.95.

### 44.2. Synthesis of tert-butyl (2S)-2-({[4-(aminomethyl)pyridin-3-yl]oxy}methyl)morpholine-4-carboxylate

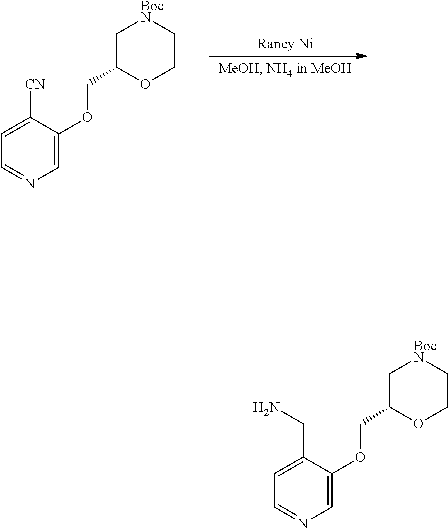 [SMILES: CC(C)(C)OC(=O)N1CCO[C@H](COc2cnccc2C#N)C1 ; "MeOH, NH4 in MeOH" ; "Raney Ni" ; CC(C)(C)OC(=O)N1CCO[C@H](COc2cnccc2CN)C1]

To a stirred mixture of tert-butyl (2S)-2-{[(4-cyanopyridin-3-yl)oxy]methyl}morpholine-4-carboxylate (1.00 g, 3.13 mmol, 1.00 equiv) and ammonia (7.0 M Solution in MeOH, 12.50 mL, 87.50 mmol) in MeOH (25.00 mL) was added Raney Ni (1.00 g, 100 w/w %) in portions at room temperature under nitrogen atmosphere. The resulting mixture was stirred for overnight at room temperature under hydrogen atmosphere. The reaction was monitored by LCMS. Desired product could be detected by LCMS. The resulting mixture was filtered, the filter cake was washed with MeOH (3×150 mL). The filtrate was concentrated under reduced pressure. The residue was purified by silica gel column chromatography, eluted with CH₂Cl₂/MeOH (20:1) to afford tert-butyl (2S)-2-({[4-(aminomethyl)pyridin-3-yl]oxy}methyl)morpholine-4-carboxylate (1.00 g, 95.79%) as a light yellow oil.

LC-MS: M+H found: 324.05.

### 44.3. Synthesis of afford tert-butyl (2S)-2-[({4-[({3-[(3-chloro-2-methoxyphenyl)carbamothioyl]-2-oxo-5,6-dihydro-1H-pyridin-4-yl}amino)methyl]pyridin-3-yl}oxy)methyl]morpholine-4-carboxylate

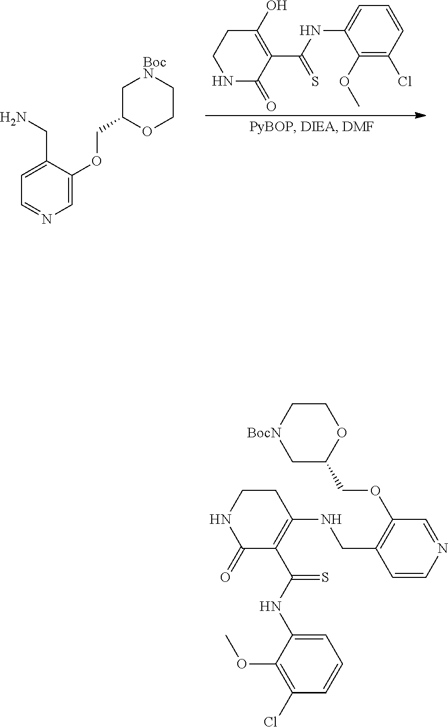

To a stirred mixture of tert-butyl (2S)-2-({[4-(aminomethyl)pyridin-3-yl]oxy}methyl)morpholine-4-carboxylate (1.10 g, 3.40 mmol, 1.00 equiv) and N-(3-chloro-2-methoxyphenyl)-4-hydroxy-2-oxo-5,6-dihydro-1H-pyridine-3-carbothioamide (1.06 g, 3.40 mmol, 1.00 equiv) in DMF (12.00 mL) were added PyBOP (2.66 g, 5.10 mmol, 1.50 equiv) and DIEA (1.32 g, 10.20 mmol, 3.00 equiv) in portions at 0 degrees C. under nitrogen atmosphere. The resulting mixture was stirred for overnight at room temperature under nitrogen atmosphere. The reaction was monitored by LCMS. Desired product could be detected by LCMS. The resulting mixture was concentrated under reduced pressure. The residue was purified by silica gel column chromatography, eluted with PE/EA (5:1) to afford tert-butyl (2S)-2-[({4-[({3-[(3-chloro-2-methoxyphenyl)carbamothioyl]-2-oxo-5,6-dihydro-1H-pyridin-4-yl}amino)methyl]pyridin-3-yl}oxy)methyl]morpholine-4-carboxylate (800 mg, 34.62%) as a yellow solid.

LC-MS: M+H found: 618.10.

### 44.4. Synthesis of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}morpholine-4-carboxylate

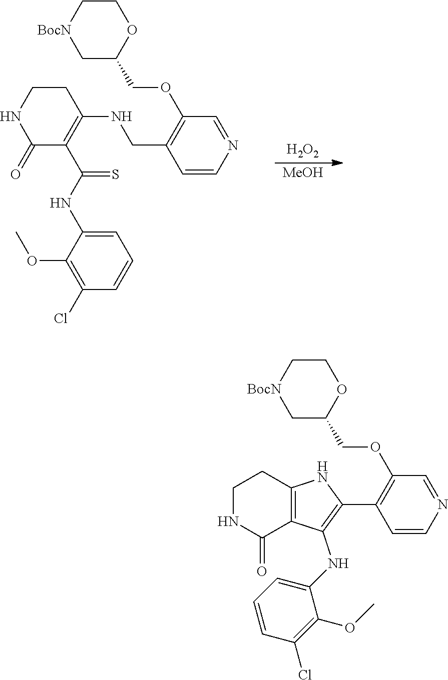 [SMILES: "MeOH" ; COc1c(Cl)cccc1NC(=S)C1=C(NCc2ccncc2OC[C@@H]2CN(C(=O)OC(C)(C)C)CCO2)CCNC1=O ; "H2O2" ; COc1c(Cl)cccc1Nc1c(-c2ccncc2OC[C@@H]2CN(C(=O)OC(C)(C)C)CCO2)[nH]c2c1C(=O)NCC2]

A mixture of tert-butyl (2S)-2-[({4-[({3-[(3-chloro-2-methoxyphenyl)carbamothioyl]-2-oxo-5,6-dihydro-1H-pyridin-4-yl}amino)methyl]pyridin-3-yl}oxy)methyl]morpholine-4-carboxylate (800 mg, 1.29 mmol, 1.00 equiv) and H₂O₂ (30w/w %, 190 mg, 1.68 mmol, 1.30 equiv) in MeOH (8.00 mL) was stirred for 4 h at 80 degrees C. under nitrogen atmosphere. The reaction was monitored by LCMS. Desired product could be detected by LCMS. The reaction was quenched with sat. sodium hyposulfite (aq.) at room temperature. The resulting mixture was concentrated under reduced pressure. The residue was purified by reverse flash chromatography with the following conditions: column, C18 spherical column; mobile phase, MeCN in water, 10% to 50% gradient in 30 min; detector, UV 254 nm to afford tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}morpholine-4-carboxylate (300 mg, 35.72%) as a light yellow solid.

LC-MS: M+H found: 584.20.

### 44.5. Synthesis of 3-[(3-chloro-2-methoxyphenyl)amino]-2-{3-[(2S)-morpholin-2-ylmethoxy]pyridin-4-yl}-1H,5H,6H,7H-pyrrolo[3,2-c]pyridin-4-one

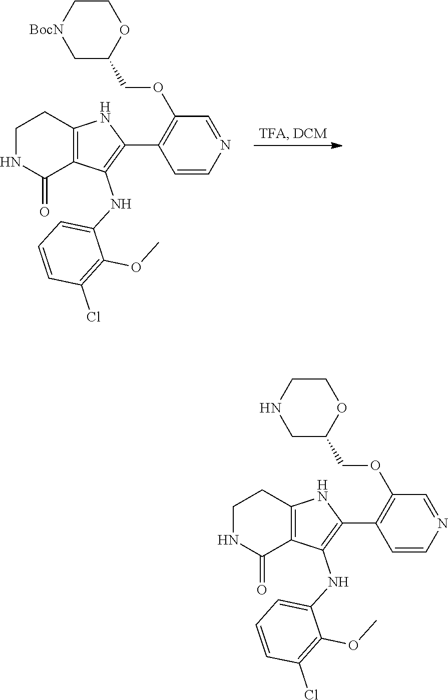 [SMILES: COc1c(Cl)cccc1Nc1c(-c2ccncc2OC[C@@H]2CN(C(=O)OC(C)(C)C)CCO2)[nH]c2c1C(=O)NCC2 ; COc1c(Cl)cccc1Nc1c(-c2ccncc2OC[C@@H]2CNCCO2)[nH]c2c1C(=O)NCC2]

To a stirred mixture of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}morpholine-4-carboxylate (250 mg, 0.43 mmol, 1.00 equiv) and DCM (5.00 mL) was added TFA (1.00 mL) in portions at room temperature under air atmosphere. The resulting mixture was stirred for 1 h at room temperature under air atmosphere. Desired product could be detected by LCMS. The reaction was monitored by LCMS. The resulting mixture was concentrated under reduced pressure. The mixture was neutralized to pH 7 with saturated NaHCO₃(aq.). The aqueous layer was extracted with CH₂Cl₂ (3×10 mL). The organic phase was concentrated under reduced pressure to afford 3-[(3-chloro-2-methoxyphenyl)amino]-2-{3-[(2S)-morpholin-2-ylmethoxy]pyridin-4-yl}-1H,5H,6H,7H-pyrrolo[3,2-c]pyridin-4-one (220 mg, 84.96%) as a light yellow solid.

LC-MS: M+H found: 484.10.

### 44.6. Synthesis of 2-(3-{[(2S)-4-acetylmorpholin-2-yl]methoxy}pyridin-4-yl)-3-[(3-chloro-2-methoxyphenyl)amino]-1H,5H,6H,7H-pyrrolo[3,2-c]pyridin-4-one

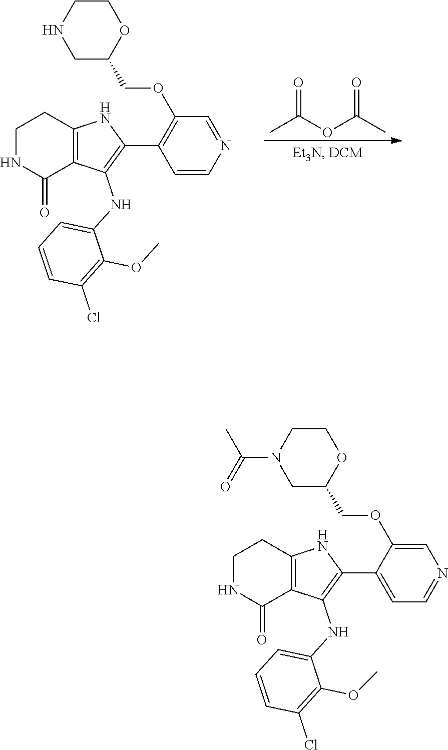

To a stirred mixture of 3-[(3-chloro-2-methoxyphenyl)amino]-2-{3-[(2S)-morpholin-2-ylmethoxy]pyridin-4-yl}-1H,5H,6H,7H-pyrrolo[3,2-c]pyridin-4-one (65 mg, 0.13 mmol, 1.00 equiv) in DCM (1.00 mL) were added Et₃N (54 mg, 0.52 mmol, 4.00 equiv) and acetic anhydride (14 mg, 0.13 mmol, 1.00 equiv) in portions at −30 degrees C. under argon atmosphere. The resulting mixture was stirred for 1 h at 0 degrees C. under argon atmosphere. The reaction was monitored by LCMS. Desired product could be detected by LCMS. The reaction was quenched with MeOH at 0 degrees C. The resulting mixture was concentrated under reduced pressure. The crude product (65 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 50% B in 7 min; Wave Length: 254 nm; RT1(min): 6.5; Number Of Runs: 0) to afford 2-(3-{[(2S)-4-acetylmorpholin-2-yl]methoxy}pyridin-4-yl)-3-[(3-chloro-2-methoxyphenyl)amino]-1H,5H,6H,7H-pyrrolo[3,2-c]pyridin-4-one (34.6 mg, 48.29%) as a off-white solid.

LC-MS: (M+H)⁺ found 525.95.

¹H NMR (300 MHz, DMSO-d6) δ 11.06 (s, 1H), 8.42 (s, 1H), 8.06-8.02 (m, 1H), 7.53 (s, 1H), 7.31-7.27 (m, 1H), 7.16 (s, 1H), 6.70-6.68 (m, 2H), 6.20-5.97 (m, 1H), 4.43-4.15 (m, 3H), 4.00-3.96 (m, 1H), 3.88 (s, 4H), 3.80-3.48 (m, 2H), 3.42-3.99 (m, 2H), 3.29-3.04 (m, 1H), 2.90-2.83 (m, 2H), 2.79-2.57 (m, 1H), 2.03 (s, 3H).

### Example 45. 3-[(3-chloro-2-methoxyphenyl)amino]-2-(3-{[(2S)-4-methanesulfonylmorpholin-2-yl]methoxy}pyridin-4-yl)-1H,5H,6H,7H-pyrrolo[3,2-c]pyridin-4-one (compound 193)

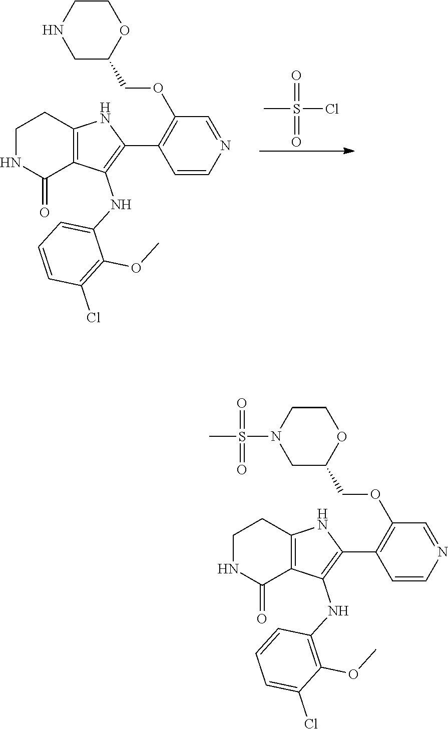

To a stirred mixture of 3-[(3-chloro-2-methoxyphenyl)amino]-2-{3-[(2S)-morpholin-2-ylmethoxy]pyridin-4-yl}-1H,5H,6H,7H-pyrrolo[3,2-c]pyridin-4-one (100 mg, 0.21 mmol, 1.00 equiv) in DCM (1.50 mL) were added Et₃N (84 mg, 0.84 mmol, 4.00 equiv) and methanesulfonyl chloride (24 mg, 0.21 mmol, 1.00 equiv) in portions at −30 degrees C. under argon atmosphere. The resulting mixture was stirred for 1 h at 0 degrees C. under argon atmosphere. The reaction was monitored by LCMS. Desired product could be detected by LCMS. The reaction was quenched with MeOH at 0 degrees C. The resulting mixture was concentrated under reduced pressure. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 23% B to 43% B in 10 min, 43% B; Wave Length: 254/220 nm; RT1(min): 9.67; Number Of Runs: 0) to afford 3-[(3-chloro-2-methoxyphenyl)amino]-2-(3-{[(2S)-4-methanesulfonylmorpholin-2-yl]methoxy}pyridin-4-yl)-1H,5H,6H,7H-pyrrolo[3,2-c]pyridin-4-one (41.3 mg, 34.49%) as a off-white solid.

LC-MS: (M+H)⁺ found: 561.90.

¹H NMR (300 MHz, DMSO-d6) δ 11.05 (s, 1H), 8.42 (s, 1H), 8.05 (d, 1H), 7.51 (s, 1H), 7.28 (d, 1H), 7.16 (t, J=2.5 Hz, 1H), 6.76-6.63 (m, 2H), 6.21-6.09 (m, 1H), 4.37-4.32 (m, 1H), 4.29-4.24 (m, 1H), 4.13-4.00 (m, 2H), 3.88 (s, 3H), 3.80-3.61 (m, 2H), 3.48-3.38 (m, 3H), 2.94-2.76 (m, 7H).

### Example 46. 3-[(3-chloro-2-methoxyphenyl)amino]-2-(3-{[(3R)-4-[(2E)-4-(dimethylamino)but-2-enoyl]morpholin-3-yl]methoxy}pyridin-4-yl)-1H,5H,6H,7H-pyrrolo[3,2-c]pyridin-4-one; trifluoroacetic acid salt (compound 197)

### 46.1. Synthesis of (E)-4-(dimethylamino)but-2-enoyl chloride

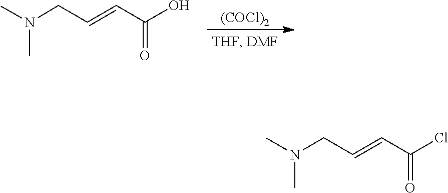

To a stirred mixture of (2E)-4-(dimethylamino)but-2-enoic acid (70 mg, 0.54 mmol, 1.00 equiv) in THF (6 mL) was added (COCl)₂ (76 mg, 0.60 mmol, 1.10 equiv) dropwise at 0 degrees C. under argon atmosphere. The resulting mixture was stirred for 30 min at room temperature under argon atmosphere. The reaction was monitored by TLC (CH₂Cl₂/MeOH=5:1). The resulting mixture was used in the next step directly without further purification.

### 46.2. Synthesis of 3-[(3-chloro-2-methoxyphenyl)amino]-2-(3-{[(3R)-4-[(2E)-4-(dimethylamino)but-2-enoyl]morpholin-3-yl]methoxy}pyridin-4-yl)-1H,5H,6H,7H-pyrrolo[3,2-c]pyridin-4-one; trifluoroacetic acid salt

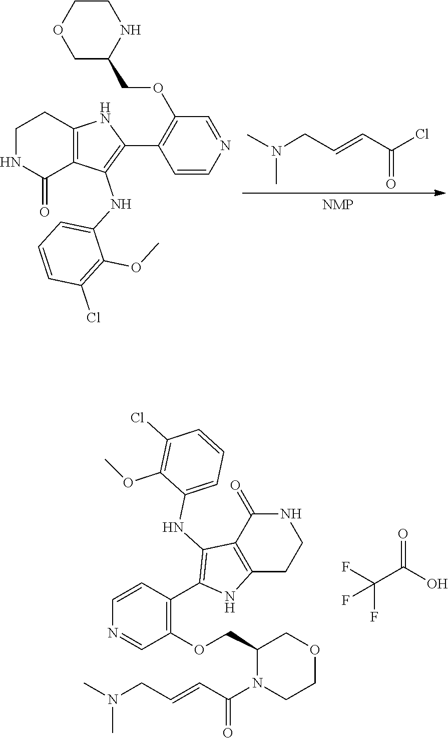

To a stirred mixture of (2E)-4-(dimethylamino)but-2-enoyl chloride (21 mg, 0.14 mmol, 1.0 equiv) in THF (0.5 mL) was added a solution of 3-[(3-chloro-2-methoxyphenyl)amino]-2-{3-[(3R)-morpholin-3-ylmethoxy]pyridin-4-yl}-1H,5H,6H,7H-pyrrolo[3,2-c]pyridin-4-one (70 mg, 0.14 mmol, 1.00 equiv) in NMP (0.5 mL) dropwise at 0° C. under nitrogen atmosphere. The resulting mixture was stirred for 1.5 h at room temperature. The reaction was monitored by TLC. The resulting mixture was concentrated under reduced pressure. The crude product (100 mg) was purified by Prep-HPLC with the following conditions (Column: Xcelect CSH F-pheny OBD Column, 19*250 mm, 5 μm; Mobile Phase A: Water (0.05% TFA), Mobile Phase B: ACN; Flow rate: 25 mL/min; Gradient: 14% B to 21% B in 10 min, 21% B; Wave Length: 254 nm; RT1(min): 7.68; Number Of Runs: 0) to afford 3-[(3-chloro-2-methoxyphenyl)amino]-2-(3-{[(3R)-4-[(2E)-4-(dimethylamino)but-2-enoyl]morpholin-3-yl]methoxy}pyridin-4-yl)-1H,5H,6H,7H-pyrrolo[3,2-c]pyridin-4-one; trifluoroacetic acid (14.4 mg, 14.04%) as a yellow solid.

LC-MS: (M+Na)⁺ found: 617.05

¹H NMR (400 MHz, DMSO-d6) δ 11.16 (s, 1H), 9.98 (s, 1H), 8.63 (s, 1H), 8.21 (d, 1H), 7.99 (s, 1H), 7.55-7.30 (m, 2H), 7.02-6.87 (m, 1H), 6.85-6.70 (m, 2H), 6.67-6.49 (m, 1H), 6.20-6.10 (m, 1H), 5.04-4.85 (m, 1H), 4.85 (t, 1H), 4.65-4.45 (m, 1H), 4.23-4.20 (m, 1H), 4.11-3.79 (m, 7H), 3.77-3.59 (m, 2H), 3.57-3.36 (m, 3H), 3.25-3.04 (m, 1H), 2.97-2.64 (m, 6H).

### Example 47. 3-[(3-fluoro-2-methoxyphenyl)amino]-2-(3-{[(3R)-4-(prop-2-enoyl)morpholin-3-yl]methoxy}pyridin-4-yl)-1H,5H,6H,7H-pyrrolo[3,2-c]pyridin-4-one (compound 189)

### 47.1. Synthesis of tert-butyl (3R)-3-{[(4-cyanopyridin-3-yl)oxy]methyl}morpholine-4-carboxylate

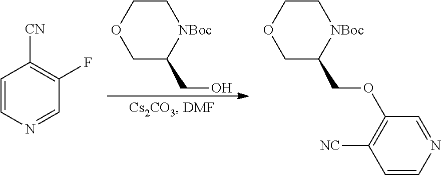

A solution of 3-fluoropyridine-4-carbonitrile (1.95 g, 15.97 mmol, 1.00 equiv) and tert-butyl (3S)-3-(hydroxymethyl)morpholine-4-carboxylate (3.47 g, 15.97 mmol, 1.00 equiv) and Cs₂CO₃ (15.61 g, 47.91 mmol, 3.00 equiv) in DMF (8 mL) was stirred for 2 h at 80 degrees C. under nitrogen atmosphere. The reaction was monitored by LCMS. The resulting mixture was diluted with water (10 mL). The resulting mixture was extracted with EtOAc (3×20 mL). The combined organic layers were washed with brine (3×5 mL), dried over anhydrous Na₂SO₄. After filtration, the filtrate was concentrated under reduced pressure. The residue was purified by silica gel column chromatography, eluted with PE/EA (2:1) to afford tert-butyl (3R)-3-{[(4-cyanopyridin-3-yl)oxy]methyl}morpholine-4-carboxylate (5 g, 98.03%) as a yellow solid.

LC-MS: (M+H)⁺ found 320.05.

### 47.2. Synthesis of tert-butyl (3R)-3-({[4-(aminomethyl)pyridin-3-yl]oxy}methyl)morpholine-4-carboxylate

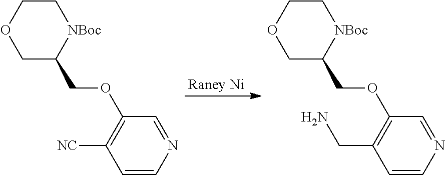 [SMILES: CC(C)(C)OC(=O)N1CCOC[C@@H]1COc1cnccc1C#N ; CC(C)(C)OC(=O)N1CCOC[C@@H]1COc1cnccc1CN]

To a solution of tert-butyl (3R)-3-{[(4-cyanopyridin-3-yl)oxy]methyl}morpholine-4-carboxylate (500 mg, 1.57 mmol, 1.00 equiv) in MeOH (5.00 mL) and ammonia (7.0 M Solution in MeOH, 5.00 mL, 35 mmol) was added Raney Ni (500 mg, 100w/w %) under nitrogen atmosphere in a 50 mL round-bottom flask. The mixture was hydrogenated at room temperature for overnight under hydrogen atmosphere using a hydrogen balloon, filtered through a Celite pad and concentrated under reduced pressure. The reaction was monitored by LCMS. The residue was purified by silica gel column chromatography, eluted with CH₂Cl₂/MeOH (20:1) to afford tert-butyl (3R)-3-({[4-(aminomethyl)pyridin-3-yl]oxy}methyl)morpholine-4-carboxylate (490 mg, 96.78%) as a yellow oil.

LC-MS: M+H found 324.00.

### 47.3. Synthesis of tert-butyl (3R)-3-({[4-({[1-(tert-butoxycarbonyl)-3-[(3-fluoro-2-methoxyphenyl)carbamothioyl]-2-oxo-5,6-dihydropyridin-4-yl]amino}methyl)pyridin-3-yl]oxy}methyl)morpholine-4-carboxylate

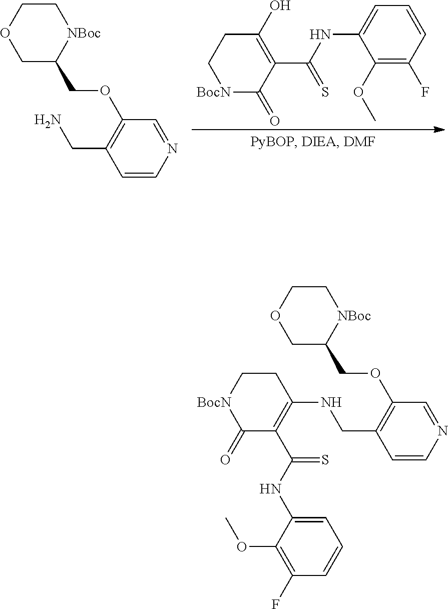 [SMILES: CC(C)(C)OC(=O)N1CCOC[C@@H]1COc1cnccc1CN ; COc1c(F)cccc1NC(=S)C1=C(O)CCN(C(=O)OC(C)(C)C)C1=O ; COc1c(F)cccc1NC(=S)C1=C(NCc2ccncc2OC[C@H]2COCCN2C(=O)OC(C)(C)C)CCN(C(=O)OC(C)(C)C)C1=O]

Into a 50 mL round-bottom flask were added tert-butyl (3R)-3-({[4-(aminomethyl)pyridin-3-yl]oxy}methyl)morpholine-4-carboxylate (490 mg, 1.50 mmol, 1.00 equiv) and tert-butyl 3-[(3-fluoro-2-methoxyphenyl)carbamothioyl]-4-hydroxy-2-oxo-5,6-dihydropyridine-1-carboxylate (600 mg, 1.50 mmol, 1.00 equiv) and PyBOP (1.18 g, 2.30 mmol, 1.50 equiv) and DIEA (587 mg, 4.50 mmol, 3.00 equiv) and DMF (15 mL) at room temperature. The resulting mixture was stirred for 5 h at room temperature under nitrogen atmosphere. The reaction was monitored by LCMS. The resulting mixture was diluted with water (20 mL). The resulting mixture was extracted with EtOAc (3×50 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EA (1:1) to afford tert-butyl (3R)-3-({[4-({[1-(tert-butoxycarbonyl)-3-[(3-fluoro-2-methoxyphenyl)carbamothioyl]-2-oxo-5,6-dihydropyridin-4-yl]amino}methyl)pyridin-3-yl]oxy}methyl)morpholine-4-carboxylate (970 mg, 91.22%) as a yellow solid

LC-MS: M+H found 702.2.

### 47.4. Synthesis of tert-butyl (3R)-3-[({4-[5-(tert-butoxycarbonyl)-3-[(3-fluoro-2-methoxyphenyl)amino]-4-oxo-1H,6H,7H-pyrrolo[3,2-c]pyridin-2-yl]pyridin-3-yl}oxy)methyl]morpholine-4-carboxylate

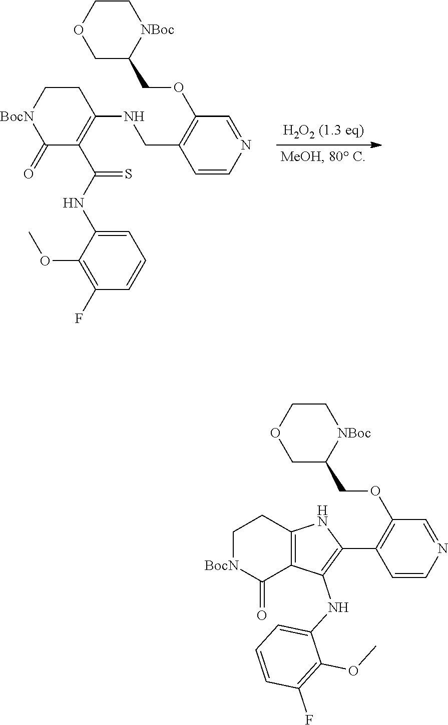 [SMILES: COc1c(F)cccc1NC(=S)C1=C(NCc2ccncc2OC[C@H]2COCCN2C(=O)OC(C)(C)C)CCN(C(=O)OC(C)(C)C)C1=O ; COc1c(F)cccc1Nc1c(-c2ccncc2OC[C@H]2COCCN2C(=O)OC(C)(C)C)[nH]c2c1C(=O)N(C(=O)OC(C)(C)C)CC2]

A solution of tert-butyl (3R)-3-({[4-({[1-(tert-butoxycarbonyl)-3-[(3-fluoro-2-methoxyphenyl)carbamothioyl]-2-oxo-5,6-dihydropyridin-4-yl]amino}methyl)pyridin-3-yl]oxy}methyl)morpholine-4-carboxylate (970 mg, 1.38 mmol, 1.00 equiv) and H₂O₂ (30w/w %, 204 mg, 1.80 mmol, 1.30 equiv) in MeOH (15 mL) was stirred for 4 h at 80 degrees C. under air atmosphere. The reaction was monitored by LCMS. The reaction was quenched by the addition of sat. Na₂SO₃ (sat.) (0.1 mL) at 0 degrees C. The resulting mixture was concentrated under reduced pressure. The residue was purified by silica gel column chromatography, eluted with CH₂Cl₂/MeOH (50:1) to afford tert-butyl (3R)-3-[({4-[5-(tert-butoxycarbonyl)-3-[(3-fluoro-2-methoxyphenyl)amino]-4-oxo-1H,6H,7H-pyrrolo[3,2-c]pyridin-2-yl]pyridin-3-yl}oxy)methyl]morpholine-4-carboxylate (600 mg, 39.01%) as a yellow solid.

LC-MS: M+H found 668.2.

### 47.5. Synthesis of 3-[(3-fluoro-2-methoxyphenyl)amino]-2-{3-[(3R)-morpholin-3-ylmethoxy]pyridin-4-yl}-1H,5H,6H,7H-pyrrolo[3,2-c]pyridin-4-one

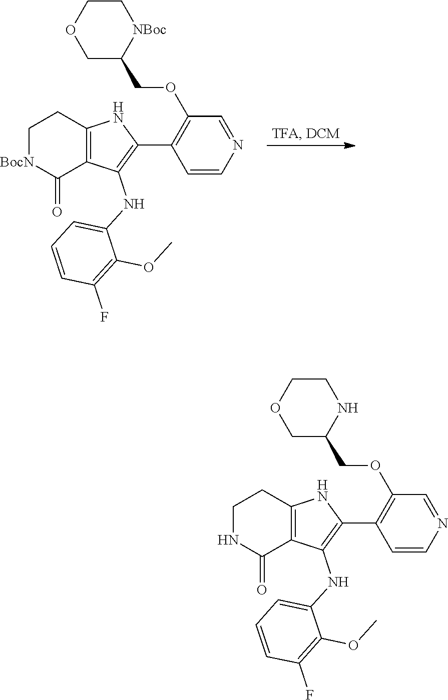 [SMILES: COc1c(F)cccc1Nc1c(-c2ccncc2OC[C@H]2COCCN2C(=O)OC(C)(C)C)[nH]c2c1C(=O)N(C(=O)OC(C)(C)C)CC2 ; COc1c(F)cccc1Nc1c(-c2ccncc2OC[C@H]2COCCN2)[nH]c2c1C(=O)NCC2]

A solution of tert-butyl (3R)-3-[({4-[5-(tert-butoxycarbonyl)-3-[(3-fluoro-2-methoxyphenyl)amino]-4-oxo-1H,6H,7H-pyrrolo[3,2-c]pyridin-2-yl]pyridin-3-yl}oxy)methyl]morpholine-4-carboxylate (300 mg, 0.45 mmol, 1.00 equiv) in TFA (1.5 mL) and DCM (4.5 mL) was stirred for 20 min at room temperature under nitrogen atmosphere. The reaction was monitored by LCMS. The resulting mixture was concentrated under reduced pressure. The residue was purified by Prep-TLC (CH₂Cl₂/MeOH 10:1) to afford 3-[(3-fluoro-2-methoxyphenyl)amino]-2-{3-[(3R)-morpholin-3-ylmethoxy]pyridin-4-yl}-1H,5H,6H,7H-pyrrolo[3,2-c]pyridin-4-one (150 mg, 49.28%) as a yellow solid.

LC-MS: M+H⁺ found 468.1.

### 47.6. Synthesis of 3-[(3-fluoro-2-methoxyphenyl)amino]-2-(3-{[(3R)-4-(prop-2-enoyl)morpholin-3-yl]methoxy}pyridin-4-yl)-1H,5H,6H,7H-pyrrolo[3,2-c]pyridin-4-one

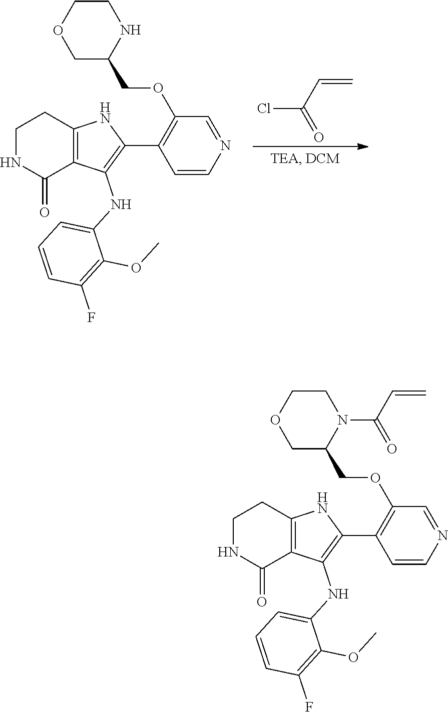

Into a 8 mL vial were added 3-[(3-fluoro-2-methoxyphenyl)amino]-2-{3-[(3R)-morpholin-3-ylmethoxy]pyridin-4-yl}-1H,5H,6H,7H-pyrrolo[3,2-c]pyridin-4-one (70 mg, 0.15 mmol, 1.00 equiv) and acryloyl chloride (12 mg, 0.14 mmol, 0.90 equiv) and TEA (45 mg, 0.45 mmol, 3.00 equiv) and DCM (1.5 mL) at 0 degrees C. The resulting mixture was stirred for 1 h at room temperature under nitrogen atmosphere. The reaction was monitored by LCMS. The resulting mixture was concentrated under vacuum. The crude product (80 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 18% B to 33% B in 11 min, 33% B; Wave Length: 254/220 nm; RT1(min): 10.38; Number Of Runs: 0) to afford 3-[(3-fluoro-2-methoxyphenyl)amino]-2-(3-{[(3R)-4-(prop-2-enoyl)morpholin-3-yl]methoxy}pyridin-4-yl)-1H,5H,6H,7H-pyrrolo[3,2-c]pyridin-4-one (16.3 mg, 20.29%) as a yellow solid.

LC-MS: M+H⁺ found: 522.00.

¹H NMR (300 MHz, DMSO-d6) δ 10.91 (s, 1H), 8.46 (s, 1H), 8.11-7.91 (m, 1H), 7.51 (s, 1H), 7.44-7.21 (m, 1H), 7.12 (s, 1H), 7.00-6.34 (m, 3H), 6.26-5.87 (m, 2H), 5.76 (s, 1H), 5.14-4.61 (m, 1H), 4.55-4.12 (m, 2H), 4.11-3.79 (m, 6H), 3.62-3.59 (m, 2H), 3.48-3.40 (m, 3H), 3.24-2.70 (m, 2H).

### Example 48. 3-[(3-chloro-2-methoxyphenyl)amino]-2-(3-{[(2R)-4-(prop-2-enoyl)morpholin-2-yl]methoxy}pyridin-4-yl)-1H,5H,6H,7H-pyrrolo[3,2-c]pyridin-4-one (compound 201)

### 48.1. Synthesis of (3-[(3-chloro-2-methoxyphenyl)amino]-2-{3-[(2R)-morpholin-2-ylmethoxy]pyridin-4-yl}-1H,5H,6H,7H-pyrrolo[3,2-c]pyridin-4-one

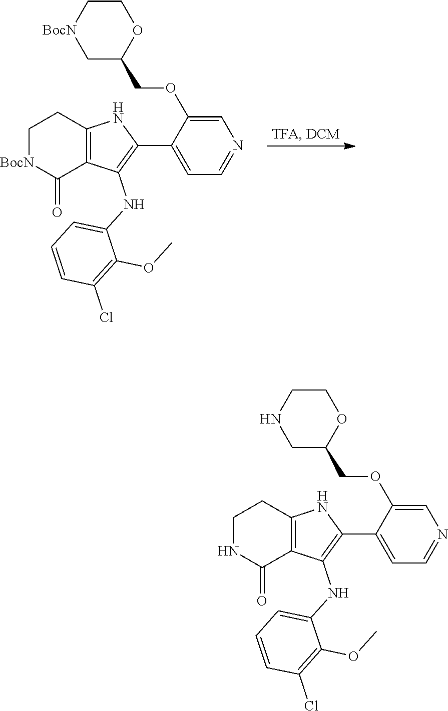 [SMILES: COc1c(Cl)cccc1Nc1c(-c2ccncc2OC[C@H]2CN(C(=O)OC(C)(C)C)CCO2)[nH]c2c1C(=O)N(C(=O)OC(C)(C)C)CC2 ; COc1c(Cl)cccc1Nc1c(-c2ccncc2OC[C@H]2CNCCO2)[nH]c2c1C(=O)NCC2]

A solution of tert-butyl (2R)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]morpholine-4-carboxylate (100 mg, 0.15 mmol, 1.00 equiv) in DCM (1.5 mL) and TFA (0.5 mL) was stirred for 20 min at room temperature under nitrogen atmosphere. The resulting mixture was concentrated under reduced pressure to afford (3-[(3-chloro-2-methoxyphenyl)amino]-2-{3-[(2R)-morpholin-2-ylmethoxy]pyridin-4-yl}-1H,5H,6H,7H-pyrrolo[3,2-c]pyridin-4-one (65 mg, 91.89%) as a brown oil, which was used in the next step directly without further purification.

LC-MS: M+H⁺ found 484.05.

### 48.2. Synthesis of 3-[(3-chloro-2-methoxyphenyl)amino]-2-(3-{[(2R)-4-(prop-2-enoyl)morpholin-2-yl]methoxy}pyridin-4-yl)-1H,5H,6H,7H-pyrrolo[3,2-c]pyridin-4-one

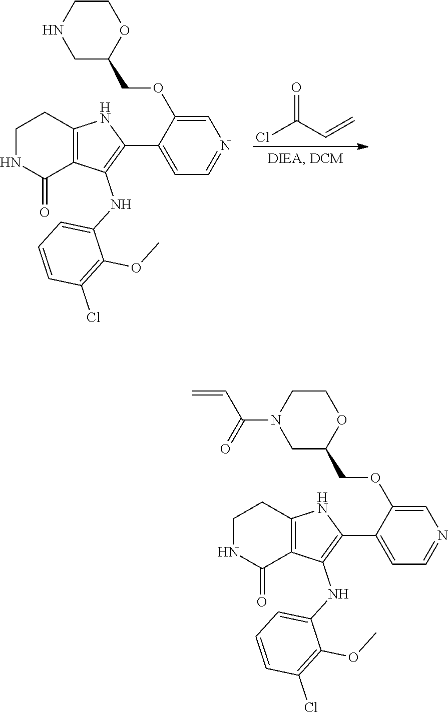

To a stirred solution of 3-[(3-chloro-2-methoxyphenyl)amino]-2-{3-[(2R)-morpholin-2-ylmethoxy]pyridin-4-yl}-1H,5H,6H,7H-pyrrolo[3,2-c]pyridin-4-one (50 mg, 0.10 mmol, 1.00 equiv) and DIEA (80 mg, 0.62 mmol, 6.00 equiv) in DCM (1 mL) was added acryloyl chloride (8 mg, 0.09 mmol, 0.9 equiv) dropwise at 0 degrees C. under nitrogen atmosphere. The resulting mixture was stirred for 10 min at room temperature under nitrogen atmosphere. The reaction was quenched by the addition of MeOH (0.5 mL) at 0 degrees C. The resulting mixture was concentrated under reduced pressure to afford crude product. The crude product (60 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 8% B to 38% B in 7 min, 38% B; Wave Length: 254/220 nm; RT1(min): 6.53; Number Of Runs: 0) to afford 3-[(3-chloro-2-methoxyphenyl)amino]-2-(3-{[(2R)-4-(prop-2-enoyl)morpholin-2-yl]methoxy}pyridin-4-yl)-1H,5H,6H,7H-pyrrolo[3,2-c]pyridin-4-one (14.2 mg, 24.45%) as a yellow solid.

LC-MS: M+H⁺ found: 537.95.

¹H NMR (300 MHz, DMSO-d6) δ 11.12 (s, 1H), 8.48 (s, 1H), 8.13 (d, 1H), 7.70 (s, 1H), 7.40 (d, 1H), 7.23 (t, 1H), 6.88-6.66 (m, 3H), 6.25-6.08 (m, 2H), 5.75-5.71 (m, 1H), 4.54-4.38 (m, 1H), 4.36-4.23 (m, 2H), 4.18-3.93 (m, 3H), 3.90 (s, 3H), 3.51 (s, 1H), 3.45 (t, 2H), 3.35-3.07 (m, 1H), 2.98-2.74 (m, 3H).

### Example 49. (6S)-6-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}morpholin-3-one (compound 194)

### 49.1. Synthesis of 3-{[(2S)-5-oxomorpholin-2-yl]methoxy}pyridine-4-carbonitrile

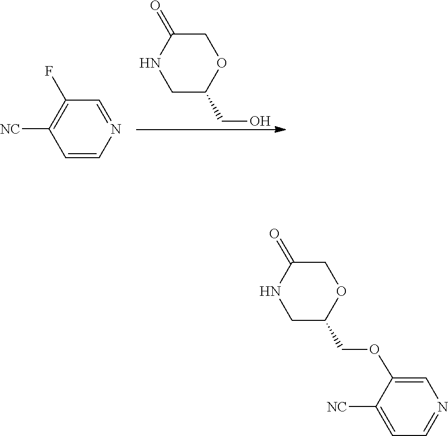 [SMILES: N#Cc1ccncc1F ; O=C1CO[C@H](CO)CN1 ; N#Cc1ccncc1OC[C@@H]1CNC(=O)CO1]

A solution of 3-fluoropyridine-4-carbonitrile (126 mg, 1.03 mmol, 1.00 equiv) and (6S)-6-(hydroxymethyl)morpholin-3-one (788 mg, 1.24 mmol, 1.20 equiv) and Cs₂CO₃ (588 mg, 3.10 mmol, 3.00 equiv) in DMF (3.00 mL) was stirred for 1 h at 60 degree C. under N₂ atmosphere. The mixture was allowed to cool down to RT. The residue was dissolved in EA (20.00 ml). The resulting mixture was washed with of saturated salt solution. The resulting mixture was concentrated under reduced pressure. The residue was purified by silica gel column chromatograph by DCM/MeOH=10/1 to afford 3-{[(2S)-5-oxomorpholin-2-yl]methoxy}pyridine-4-carbonitrile (150 mg, 61.98%) as a white solid.

LC-MS: M+H found: 233.90.

### 49.2. Synthesis of (S)-6-(((4-(aminomethyl)pyridin-3-yl)oxy)methyl)morpholin-3-one

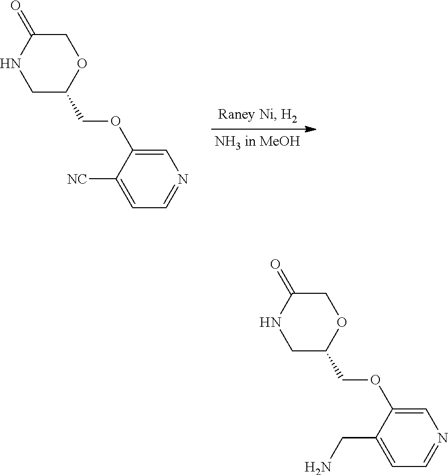 [SMILES: N#Cc1ccncc1OC[C@@H]1CNC(=O)CO1 ; NCc1ccncc1OC[C@@H]1CNC(=O)CO1]

A solution of 3-{[(2S)-5-oxomorpholin-2-yl]methoxy}pyridine-4-carbonitrile (1.02 g, 4.38 mmol, 1.00 equiv) and Raney Ni (1.02 g, 100 w/w %) in a solution of Ammonia (7.0 M Solution in MeOH, 5.00 mL, 35.00 mmol) in MeOH (10.00 mL) was stirred for 1 h at RT under H2 atmosphere. The reaction was monitored by LCMS. The crude product (1.00 g) was used in the next step directly without further purification.

LC-MS: M+H found: 238.27.

### 49.3. Synthesis of tert-butyl 3-[(3-chloro-2-methoxyphenyl)carbamothioyl]-2-oxo-4-{[(3-{[(2S)-5-oxomorpholin-2-yl]methoxy}pyridin-4-yl)methyl]amino}-5,6-dihydropyridine-1-carboxylate

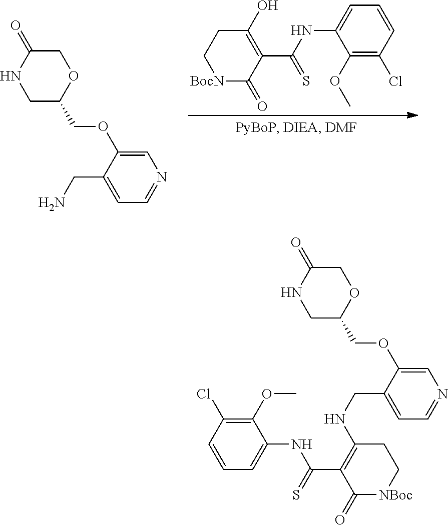 [SMILES: NCc1ccncc1OC[C@@H]1CNC(=O)CO1 ; COc1c(Cl)cccc1NC(=S)C1=C(O)CCN(C(=O)OC(C)(C)C)C1=O ; COc1c(Cl)cccc1NC(=S)C1=C(NCc2ccncc2OC[C@@H]2CNC(=O)CO2)CCN(C(=O)OC(C)(C)C)C1=O]

To a stirred solution of (6S)-6-({[4-(aminomethyl)pyridin-3-yl]oxy}methyl)morpholin-3-one (400 mg, 1.68 mmol, 1.00 equiv) and tert-butyl 3-[(3-chloro-2-methoxyphenyl)carbamothioyl]-4-hydroxy-2-oxo-5,6-dihydropyridine-1-carboxylate (696 mg, 1.68 mmol, 1.00 equiv) in DMF (16.00 mL) was added DIEA (654 mg, 5.06 mmol, 3.00 equiv) and PyBoP (1.31 g, 2.53 mmol, 1.50 equiv) dropwise at RT under N₂ atmosphere. The resulting mixture was stirred for 2 h at RT under N₂ atmosphere. The reaction was monitored by LCMS. The resulting mixture was extracted with EA (10.00 mL). The combined organic layers were washed with saturated salt solution (10.00 mL), dried over anhydrous Na₂SO₄. After filtration, the filtrate was concentrated under reduced pressure. The residue was purified by silica gel column chromatography, eluted with MeOH/DCM (10%) to afford tert-butyl 3-[(3-chloro-2-methoxyphenyl)carbamothioyl]-2-oxo-4-{[(3-{[(2S)-5-oxomorpholin-2-yl]methoxy}pyridin-4-yl)methyl]amino}-5,6-dihydropyridine-1-carboxylate (450 mg, 38.00%) as a dark yellow solid.

LC-MS: M+H found: 633.14.

### 49.4. Synthesis of tert-butyl 3-[(3-chloro-2-methoxyphenyl)amino]-4-oxo-2-(3-{[(2S)-5-oxomorpholin-2-yl]methoxy}pyridin-4-yl)-1H,6H,7H-pyrrolo[3,2-c]pyridine-5-carboxylate

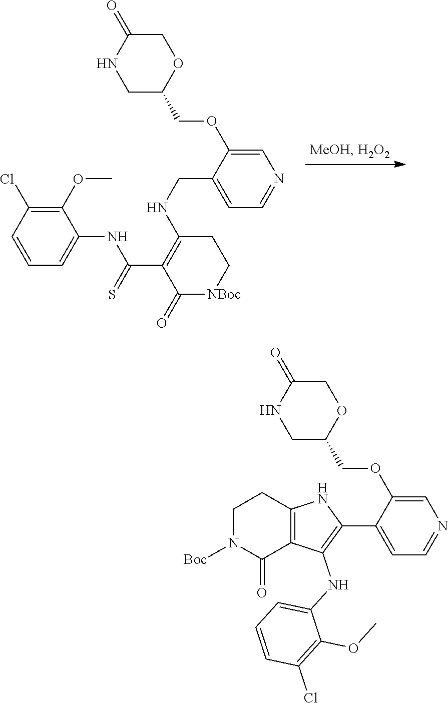 [SMILES: COc1c(Cl)cccc1NC(=S)C1=C(NCc2ccncc2OC[C@@H]2CNC(=O)CO2)CCN(C(=O)OC(C)(C)C)C1=O ; COc1c(Cl)cccc1Nc1c(-c2ccncc2OC[C@@H]2CNC(=O)CO2)[nH]c2c1C(=O)N(C(=O)OC(C)(C)C)CC2]

Into a MeOH (10.00 mL) were added tert-butyl 3-[(3-chloro-2-methoxyphenyl)carbamothioyl]-2-oxo-4-{[(3-{[(2S)-5-oxomorpholin-2-yl]methoxy}pyridin-4-yl)methyl]amino}-5,6-dihydropyridine-1-carboxylate (400 mg, 0.63 mmol, 1.00 equiv) and H₂O₂ solution (30w/w %, 70 mg, 0.63 mmol, 1.00 equiv) at RT. The resulting mixture was stirred for 1 h at 80 degrees C. The reaction was monitored by LCMS. The residue was concentrated under reduced pressure. The residue was purified by silica gel column chromatography, eluted with MeOH/DCM (10%) to afford tert-butyl 3-[(3-chloro-2-methoxyphenyl)amino]-4-oxo-2-(3-{[(2S)-5-oxomorpholin-2-yl]methoxy}pyridin-4-yl)-1H,6H,7H-pyrrolo[3,2-c]pyridine-5-carboxylate (120 mg, 28.54%) as a yellow oil.

LC-MS: M+H found: 599.06.

### 49.5. Synthesis of (6S)-6-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}morpholin-3-one

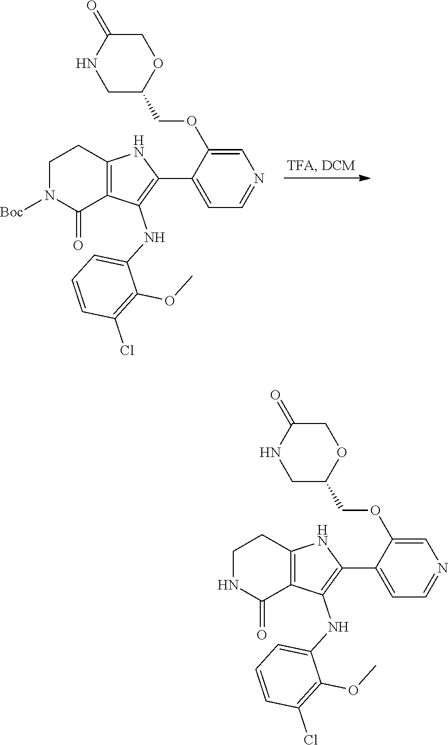 [SMILES: COc1c(Cl)cccc1Nc1c(-c2ccncc2OC[C@@H]2CNC(=O)CO2)[nH]c2c1C(=O)N(C(=O)OC(C)(C)C)CC2 ; COc1c(Cl)cccc1Nc1c(-c2ccncc2OC[C@@H]2CNC(=O)CO2)[nH]c2c1C(=O)NCC2]

To a stirred solution of tert-butyl 3-[(3-chloro-2-methoxyphenyl)amino]-4-oxo-2-(3-{[(2S)-5-oxomorpholin-2-yl]methoxy}pyridin-4-yl)-1H,6H,7H-pyrrolo[3,2-c]pyridine-5-carboxylate (120 mg, 0.20 mmol, 1.00 equiv) in DCM (1.00 mL) was added TFA (1.00 mL) at room temperature. The resulting mixture was stirred for 1 h at room temperature. The reaction was monitored by LCMS. The crude product was purified by Prep-HPLC with the following conditions (Column: Xselect CSH F-Phenyl OBD column, 19*250 mm, 5 μm; Mobile Phase A: Water (0.05% TFA), Mobile Phase B: ACN; Flow rate: 25 mL/min; Gradient: 21% B to 32% B in 10 min, 32% B; Wave Length: 254 nm; RT1(min): 8.75; Number Of Runs: 0) to afford (6S)-6-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}morpholin-3-one (19.0 mg, 18.35%) as a yellow solid.

LC-MS: (M+H)⁺ found 497.90.

¹H NMR (400 MHz, DMSO-d6) δ11.29 (s, 1H), 8.53 (s, 1H), 8.22 (d, 1H), 8.12 (d, 1H), 7.89 (s, 1H), 7.48 (d, 1H), 7.32 (s, 1H), 6.83-6.71 (m, 2H), 6.16 (d, 1H), 4.52-4.41 (m, 1H), 4.38-4.25 (m, 2H), 4.18 (s, 2H), 3.39 (s, 3H), 3.29-3.26 (m, 4H), 2.96-2.88 (m, 2H).

### Example 50. 3-[(3-chloro-2-methoxyphenyl)amino]-2-(3-{[(2R)-1-methanesulfonylazetidin-2-yl]methoxy}pyridin-4-yl)-1H,5H,6H,7H-pyrrolo[3,2-c]pyridin-4-one (compound 191)

### 50.1. Synthesis of tert-butyl (2R)-2-{[(4-cyanopyridin-3-yl)oxy]methyl}azetidine-1-carboxylate

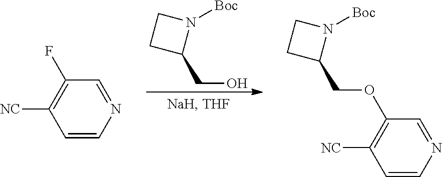 [SMILES: N#Cc1ccncc1F ; CC(C)(C)OC(=O)N1CC[C@@H]1CO ; CC(C)(C)OC(=O)N1CC[C@@H]1COc1cnccc1C#N]

To a stirred solution of tert-butyl (2R)-2-(hydroxymethyl)azetidine-1-carboxylate (0.74 g, 3.93 mmol, 1.00 equiv) in THF (7.00 mL) was added NaH (0.24 g, 5.90 mmol, 1.50 equiv, 60%) at 0° C. and stirred for 20 minutes. To the above mixture was added dropwise a solution of 3-fluoropyridine-4-carbonitrile (0.48 g, 3.93 mmol, 1.00 equiv) in THF (7.00 mL) at 0° C. The resulting mixture was stirred for additional 0.5 h at 0° C. The resulting mixture was quenched with water and concentrated under vacuum. The residue was purified by silica gel column chromatography, eluted with PE/EA=3:1 to afford tert-butyl (2R)-2-{[(4-cyanopyridin-3-yl)oxy]methyl}azetidine-1-carboxylate (1.09 g, 95.83%) as a colourless oil.

LC-MS: (M+H)⁺ found: 290.2.

### 50.2. Synthesis of tert-butyl (2R)-2-([[4-(aminomethyl)pyridin-3-yl]oxy]methyl)azetidine-1-carboxylate

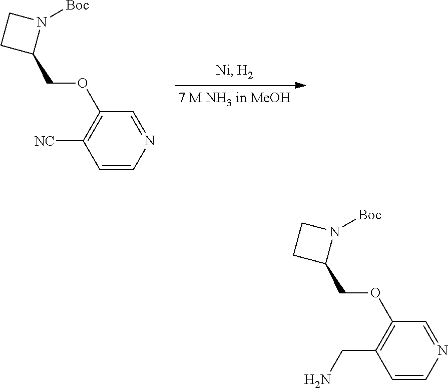 [SMILES: CC(C)(C)OC(=O)N1CC[C@@H]1COc1cnccc1C#N ; CC(C)(C)OC(=O)N1CC[C@@H]1COc1cnccc1CN]

To a solution of tert-butyl (2R)-2-[[(4-cyanopyridin-3-yl)oxy]methyl]azetidine-1-carboxylate (1.39 g, 4.81 mmol, 1.00 equiv) in 7 M NH₃(g) in MeOH (2.00 mL) was added Raney-Ni (618 mg, 44 w/w %) at room temperature. The resulting mixture was stirred for overnight at room temperature under hydrogen atmosphere. The resulting mixture was filtered, the filter cake was washed with MeOH, The filtrate was concentrated under reduced pressure. The residue was purified by Prep-TLC (CH₂Cl₂/MeOH 10:1) to afford tert-butyl (2R)-2-([[4-(aminomethyl)pyridin-3-yl]oxy]methyl)azetidine-1-carboxylate (1.40 g, 90%) as a yellow oil.

LC-MS: (M+H)⁺ found 294.2.

### 50.3. Synthesis of tert-butyl 4-{[(3-{[(2R)-1-(tert-butoxycarbonyl)azetidin-2-yl]methoxy}pyridin-4-yl)methyl]amino}-3-[(3-chloro-2-methoxyphenyl)carbamothioyl]-2-oxo-5,6-dihydropyridine-1-carboxylate

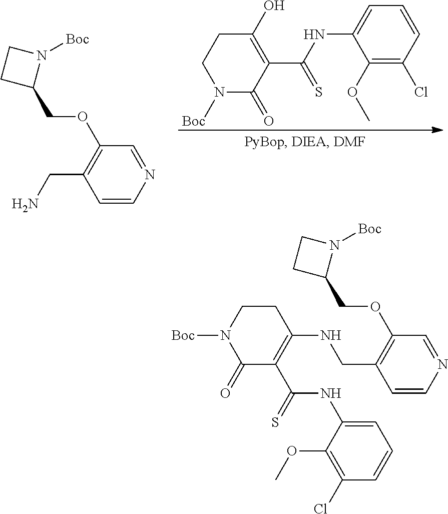 [SMILES: CC(C)(C)OC(=O)N1CC[C@@H]1COc1cnccc1CN ; COc1c(Cl)cccc1NC(=S)C1=C(O)CCN(C(=O)OC(C)(C)C)C1=O ; COc1c(Cl)cccc1NC(=S)C1=C(NCc2ccncc2OC[C@H]2CCN2C(=O)OC(C)(C)C)CCN(C(=O)OC(C)(C)C)C1=O]

To a stirred solution of tert-butyl (2R)-2-({[4-(aminomethyl)pyridin-3-yl]oxy}methyl)azetidine-1-carboxylate (630 mg, 2.15 mmol, 1.00 equiv) and tert-butyl 3-[(3-chloro-2-methoxyphenyl)carbamothioyl]-4-hydroxy-2-oxo-5,6-dihydropyridine-1-carboxylate (975 mg, 2.36 mmol, 1.10 equiv) and PyBOP (1676 mg, 3.22 mmol, 1.50 equiv) in DMF (15.00 mL) were added dropwise a solution of DIEA (833 mg, 6.44 mmol, 3.00 equiv) in DMF (15.00 mL) at RT under Ar atmosphere for 2 h. The resulting mixture was concentrated under vacuum. The residue was purified by silica gel column chromatography, eluted with DCM/MeOH=100/1 to afford tert-butyl 4-{[(3-{[(2R)-1-(tert-butoxycarbonyl)azetidin-2-yl]methoxy}pyridin-4-yl)methyl]amino}-3-[(3-chloro-2-methoxyphenyl)carbamothioyl]-2-oxo-5,6-dihydropyridine-1-carboxylate (1.494 g, 101.08%) as a yellow oil.

LC-MS: (M+H)⁺ found 688.2

### 50.4. Synthesis of tert-butyl (2R)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]azetidine-1-carboxylate

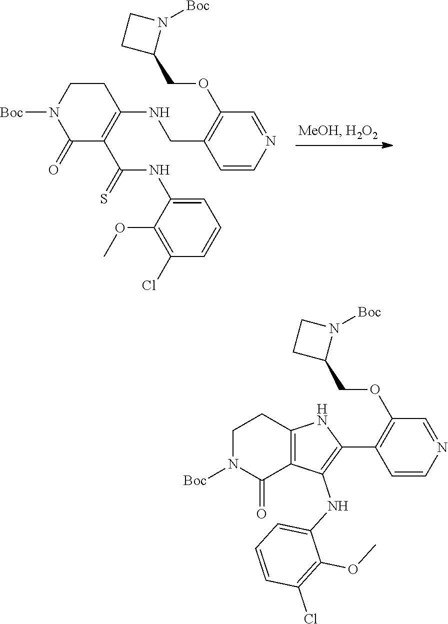 [SMILES: COc1c(Cl)cccc1NC(=S)C1=C(NCc2ccncc2OC[C@H]2CCN2C(=O)OC(C)(C)C)CCN(C(=O)OC(C)(C)C)C1=O ; COc1c(Cl)cccc1Nc1c(-c2ccncc2OC[C@H]2CCN2C(=O)OC(C)(C)C)[nH]c2c1C(=O)N(C(=O)OC(C)(C)C)CC2]

A solution of tert-butyl 4-{[(3-{[(2R)-1-(tert-butoxycarbonyl)azetidin-2-yl]methoxy}pyridin-4-yl)methyl]amino}-3-[(3-chloro-2-methoxyphenyl)carbamothioyl]-2-oxo-5,6-dihydropyridine-1-carboxylate (500 mg, 0.73 mmol, 1.00 equiv) and H₂O₂ (82 mg, 0.73 mmol, 1.00 equiv, 30%) in MeOH (10.00 mL) was stirred for 1 h at 80° C. under N₂ atmosphere. The resulting mixture was concentrated under reduced pressure. The residue was purified by reverse flash chromatography with the following conditions: column, C18 silica gel; mobile phase, A·H₂O (0.05% NH₄HCO₃). Mobile Phase B·CH₃CN; Flow rate: 60 mL/min; Gradient: 40 B to 55 B in 8 min; 254 nm; RT: 6. to afford tert-butyl (2R)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]azetidine-1-carboxylate (200 mg, 42.08%) as a yellow solid.

LC-MS: (M+H)⁺ found 654.3

### 50.5. Synthesis of 2-{3-[(2R)-azetidin-2-ylmethoxy]pyridin-4-yl}-3-[(3-chloro-2-methoxyphenyl)amino]-1H,5H,6H,7H-pyrrolo[3,2-c]pyridin-4-one

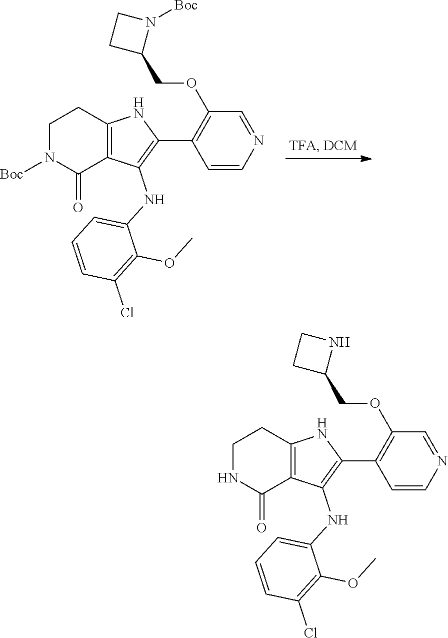 [SMILES: COc1c(Cl)cccc1Nc1c(-c2ccncc2OC[C@H]2CCN2C(=O)OC(C)(C)C)[nH]c2c1C(=O)N(C(=O)OC(C)(C)C)CC2 ; COc1c(Cl)cccc1Nc1c(-c2ccncc2OC[C@H]2CCN2)[nH]c2c1C(=O)NCC2]

A solution of tert-butyl (2R)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]azetidine-1-carboxylate (200 mg, 0.31 mmol, 1.00 equiv) and TFA (0.45 mL) in DCM (3.00 mL) was stirred for 1 h at RT. The resulting mixture was concentrated under reduced pressure. This resulted in crude product 2-{3-[(2R)-azetidin-2-ylmethoxy]pyridin-4-yl}-3-[(3-chloro-2-methoxyphenyl)amino]-1H,5H,6H,7H-pyrrolo[3,2-c]pyridin-4-one (120 mg) as a colourless oil.

LC-MS: (M+H)⁺ found 454.0

### 50.6. Synthesis of 3-[(3-chloro-2-methoxyphenyl)amino]-2-(3-{[(2R)-1-methanesulfonylazetidin-2-yl]methoxy}pyridin-4-yl)-1H,5H,6H,7H-pyrrolo[3,2-c]pyridin-4-one

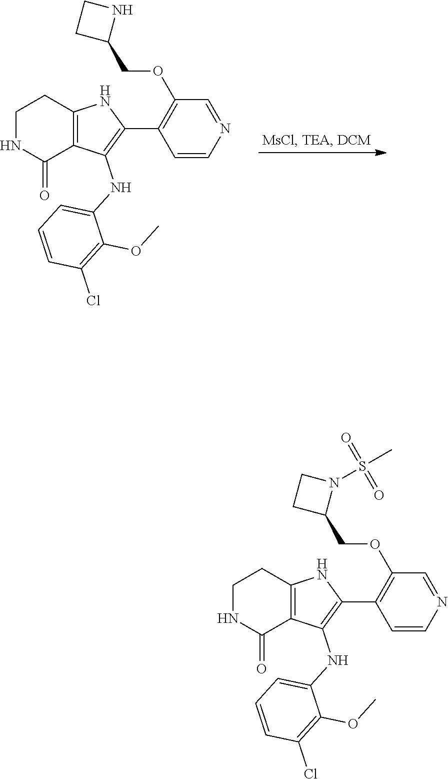 [SMILES: COc1c(Cl)cccc1Nc1c(-c2ccncc2OC[C@H]2CCN2)[nH]c2c1C(=O)NCC2 ; COc1c(Cl)cccc1Nc1c(-c2ccncc2OC[C@H]2CCN2S(C)(=O)=O)[nH]c2c1C(=O)NCC2]

A solution of tert-butyl (2R)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}azetidine-1-carboxylate (120 mg, 0.22 mmol, 1.00 equiv) and MsCl (25 mg, 0.22 mmol, 1.00 equiv) and TEA (109 mg, 1.10 mmol, 5.00 equiv) in DCM (3.00 mL) was stirred for 1 h at RT under N₂ atmosphere. Desired product could be detected by LCMS. The resulting mixture was concentrated under reduced pressure. The residue was purified by Prep-HPLC with the following conditions: (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 30% B to 60% B in 10 min, 60% B; Wave Length: 254/220 nm; RT1(min): 9.67; Number Of Runs: 0) to afford 3-[(3-chloro-2-methoxyphenyl)amino]-2-(3-{[(2R)-1-methanesulfonylazetidin-2-yl]methoxy}pyridin-4-yl)-1H,5H,6H,7H-pyrrolo[3,2-c]pyridin-4-one (20.8 mg, 17.89%) as a yellow solid.

LC-MS: (M)⁺ found 531.90

¹H NMR (400 MHz, DMSO-d6) δ 10.98 (s, 1H), 8.44 (s, 1H), 8.04 (d, 1H), 7.54 (s, 1H), 7.33 (d, 1H), 7.16 (s, 1H), 6.74-6.65 (m, 2H), 6.20-6.12 (m, 1H), 4.86-4.75 (m, 1H), 4.54-4.44 (m, 1H), 4.32-4.23 (m, 1H), 4.09-3.98 (m, 1H), 3.89 (s, 3H), 3.76-3.66 (m, 1H), 3.45-3.36 (m, 2H), 3.12 (s, 3H), 2.88 (t, 2H), 2.43-2.20 (m, 2H).

### Example 51. 3-[(3-chloro-2-methoxyphenyl)amino]-2-(3-{[(2S)-4-(prop-2-enoyl)morpholin-2-yl]methoxy}pyridin-4-yl)-1H,5H,6H,7H-pyrrolo[3,2-c]pyridin-4-one (compound 200)

### 51.1. Synthesis of (S)-3-((3-chloro-2-methoxyphenyl)amino)-2-(3-(morpholin-2-ylmethoxy)pyridin-4-yl)-1,5,6,7-tetrahydro-4H-pyrrolo[3,2-c]pyridin-4-one

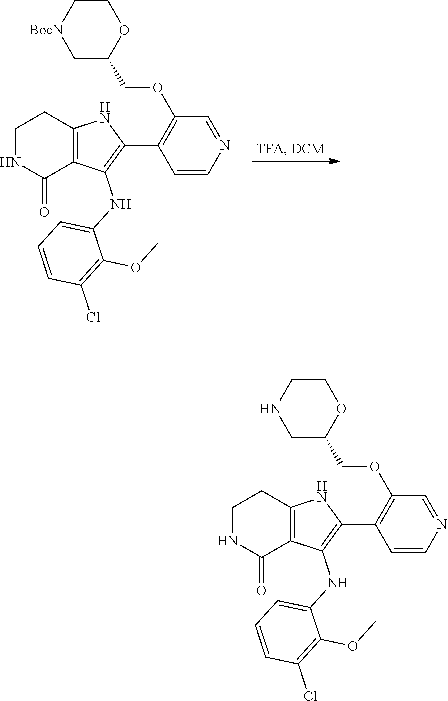 [SMILES: COc1c(Cl)cccc1Nc1c(-c2ccncc2OC[C@@H]2CN(C(=O)OC(C)(C)C)CCO2)[nH]c2c1C(=O)NCC2 ; COc1c(Cl)cccc1Nc1c(-c2ccncc2OC[C@@H]2CNCCO2)[nH]c2c1C(=O)NCC2]

To a stirred mixture of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}morpholine-4-carboxylate (50 mg, 0.09 mmol, 1.00 equiv) in DCM (0.9 mL) was added TFA (0.4 mL) dropwise at 0 degrees C. under nitrogen atmosphere. The resulting mixture was stirred for 1 h at room temperature under nitrogen atmosphere. Desired product could be detected by LCMS. The resulting mixture was concentrated under reduced pressure. The crude product was used in the next step directly without further purification.

LC-MS: (M+H)⁺ found 484.05.

### 51.2. Synthesis of 3-[(3-chloro-2-methoxyphenyl)amino]-2-(3-{[(2S)-4-(prop-2-enoyl)morpholin-2-yl]methoxy}pyridin-4-yl)-1H,5H,6H,7H-pyrrolo[3,2-c]pyridin-4-one

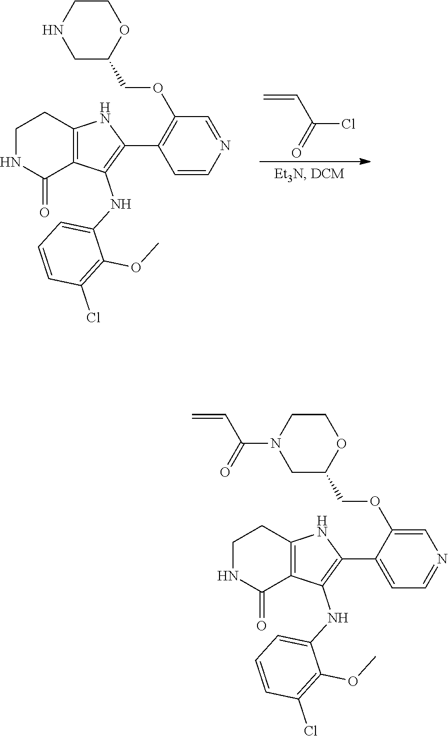

To a stirred mixture of 3-[(3-chloro-2-methoxyphenyl)amino]-2-{3-[(2S)-morpholin-2-ylmethoxy]pyridin-4-yl}-1H,5H,6H,7H-pyrrolo[3,2-c]pyridin-4-one (50 mg, 0.10 mmol, 1.00 equiv) and Et₃N (52 mg, 0.50 mmol, 5.00 equiv) in DCM (1.00 mL) was added acryloyl chloride (2 mg, 0.02 mmol, 0.15 equiv) dropwise at 0 degrees C. under argon atmosphere. The resulting mixture was stirred for 10 min at 0 degrees C. under argon atmosphere. Desired product could be detected by LCMS. The resulting mixture was concentrated under reduced pressure. The crude product (80 mg) was purified by Prep-HPLC with the following conditions (Column: Xselect Peptide CSH C18 19*150 mm 5 μm, 1; Mobile Phase A: Water (0.05% FA), Mobile Phase B: ACN; Flow rate: 60 mL/min; Gradient: 15% B to 45% B in 7 min; Wave Length: 254 nm; RT1(min): 6.5; Number Of Runs: 0) to afford 3-[(3-chloro-2-methoxyphenyl)amino]-2-(3-{[(2S)-4-(prop-2-enoyl)morpholin-2-yl]methoxy}pyridin-4-yl)-1H,5H,6H,7H-pyrrolo[3,2-c]pyridin-4-one (8.7 mg, 15.17%) as a yellow solid.

LC-MS: (M+H)⁺ found 538.30.

¹H NMR (400 MHz, DMSO-d6) δ 11.06 (s, 1H), 8.43 (s, 1H), 8.04 (s, 1H), 7.52 (d, 1H), 7.29 (s, 1H), 7.15 (s, 1H), 6.75-6.90 (m, 1H), 6.68 (d, 2H), 6.20-6.09 (m, 2H), 5.80-5.70 (m, 1H), 4.50-4.35 (m, 1H), 4.35-4.20 (m, 2H), 4.20-3.95 (m, 2H), 3.90-3.75 (m, 4H), 3.65-3.50 (m, 1H), 3.50-3.40 (m, 2H), 3.20-3.05 (m, 1H), 2.96-2.71 (m, 3H).

### Example 52. 3-[(3-chloro-2-methoxyphenyl)amino]-2-(3-{[(2S)-1-methanesulfonylazetidin-2-yl]methoxy}pyridin-4-yl)-1H,5H,6H,7H-pyrrolo[3,2-c]pyridin-4-one (compound 192)

### 52.1. Synthesis of afford tert-butyl (2S)-2-{[(4-cyanopyridin-3-yl)oxy]methyl}azetidine-1-carboxylate

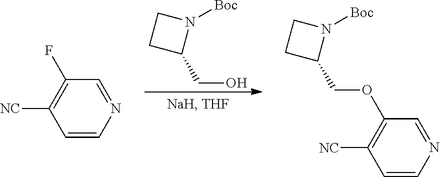 [SMILES: N#Cc1ccncc1F ; CC(C)(C)OC(=O)N1CC[C@H]1CO ; CC(C)(C)OC(=O)N1CC[C@H]1COc1cnccc1C#N]

To a stirred mixture of tert-butyl (2S)-2-(hydroxymethyl) azetidine-1-carboxylate (1533 mg, 8.19 mmol, 1.00 equiv) and NaH (60% in oil, 491 mg, 12.29 mmol, 1.50 equiv) in THF (14.00 mL) in portions at 0 degrees C. under nitrogen atmosphere. The resulting mixture was stirred for 30 min at 0 degrees C. under nitrogen atmosphere. To the above mixture was added 3-fluoropyridine-4-carbonitrile (1000 mg, 8.19 mmol, 1.00 equiv) in portions over 1 min at 0 degrees C. The resulting mixture was stirred for overnight at room temperature under nitrogen atmosphere. Desired product could be detected by LCMS. The resulting mixture was concentrated under reduced pressure. The residue was purified by silica gel column chromatography, eluted with PE/EA (1:1) to afford tert-butyl (2S)-2-{[(4-cyanopyridin-3-yl)oxy]methyl}azetidine-1-carboxylate (1800 mg, 75.96%) as a white solid.

LC-MS: (M+H)⁺ found 290.15

### 52.2. Synthesis of tert-butyl (2S)-2-({[4-(aminomethyl)pyridin-3-yl]oxy}methyl)azetidine-1-carboxylate

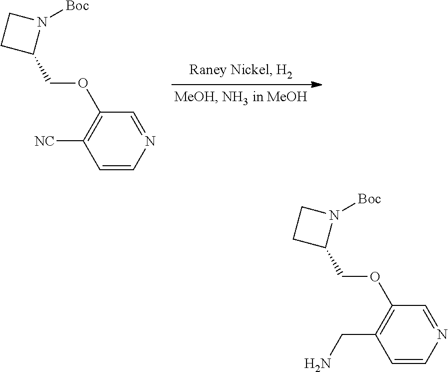 [SMILES: CC(C)(C)OC(=O)N1CC[C@H]1COc1cnccc1C#N ; CC(C)(C)OC(=O)N1CC[C@H]1COc1cnccc1CN]

To a stirred mixture of tert-butyl (2S)-2-{[(4-cyanopyridin-3-yl)oxy]methyl}azetidine-1-carboxylate (1.00 g, 3.46 mmol, 1.00 equiv) and NH₃(g) in methanol (7M in methanol, 20 mL, 140.00 mmol) was added raney nickel (0.60 g, 60 w/w %) in portions at room temperature under argon atmosphere. The resulting mixture was stirred for 2.5 h at room temperature under hydrogen atmosphere. Desired product could be detected by LCMS. The resulting mixture was filtered; the filter cake was washed with methanol (3×100 mL). The filtrate was concentrated under reduced pressure. The residue was purified by silica gel column chromatography, eluted with CH₂Cl₂/MeOH (15:1) to afford tert-butyl (2S)-2-({[4-(aminomethyl)pyridin-3-yl]oxy}methyl)azetidine-1-carboxylate (1.1 g, 90.41%) as a clear oil.

LC-MS: (M+H)⁺ found 294.20

### 52.3. Synthesis of tert-butyl 2-[({4-[({3-[(3-chloro-2-methoxyphenyl)carbamothioyl]-2-oxo-5,6-dihydro-1H-pyridin-4-yl}amino)methyl]pyridin-3-yl}oxy)methyl]azetidine-1-carboxylate

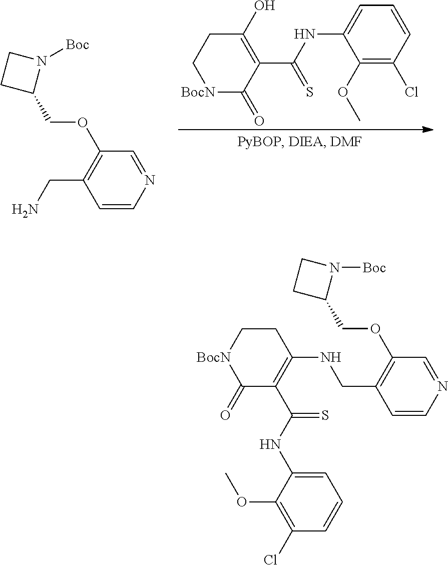 [SMILES: CC(C)(C)OC(=O)N1CC[C@H]1COc1cnccc1CN ; COc1c(Cl)cccc1NC(=S)C1=C(O)CCN(C(=O)OC(C)(C)C)C1=O ; COc1c(Cl)cccc1NC(=S)C1=C(NCc2ccncc2OC[C@@H]2CCN2C(=O)OC(C)(C)C)CCN(C(=O)OC(C)(C)C)C1=O]

To a stirred mixture of tert-butyl (2S)-2-({[4-(aminomethyl)pyridin-3-yl]oxy}methyl)azetidine-1-carboxylate (1.00 g, 3.41 mmol, 1.00 equiv) and tert-butyl 3-[(3-chloro-2-methoxyphenyl)carbamothioyl]-4-hydroxy-2-oxo-5,6-dihydropyridine-1-carboxylate (1.40 g, 3.41 mmol, 1.00 equiv) and PyBOP (2.66 g, 5.11 mmol, 1.50 equiv) in DMF (20.00 mL) was added DIEA (1.32 g, 10.23 mmol, 3.00 equiv) in portions at room temperature under nitrogen atmosphere. The resulting mixture was stirred for overnight at room temperature under nitrogen atmosphere. Desired product could be detected by LCMS. The resulting mixture was filtered; the filter cake was washed with ethyl acetate (3×40 mL). The filtrate was concentrated under reduced pressure. The residue was purified by reverse flash chromatography with the following conditions: column, silica gel; mobile phase, MeCN in water, 10% to 50% gradient in 10 min; detector, UV 254 nm. This resulted in tert-butyl 2-[({4-[({3-[(3-chloro-2-methoxyphenyl)carbamothioyl]-2-oxo-5,6-dihydro-1H-pyridin-4-yl}amino)methyl]pyridin-3-yl}oxy)methyl]azetidine-1-carboxylate (1.1 g, 54.87%) as a yellow solid.

LC-MS: (M+H)⁺ found 688.20

### 52.4. Synthesis of tert-butyl (2S)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]azetidine-1-carboxylate

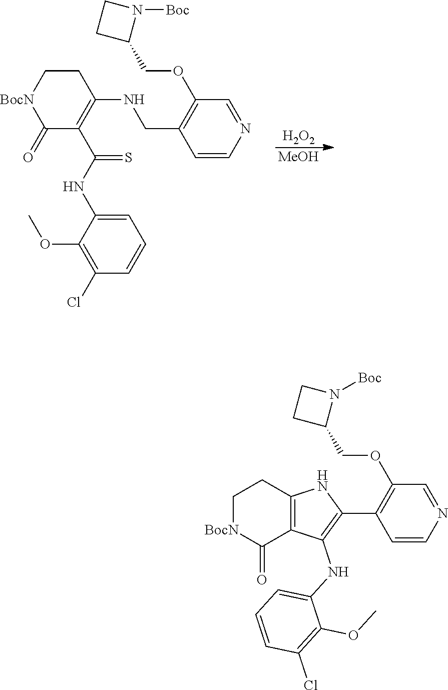

To a stirred mixture of tert-butyl 4-{[(3-{[1-(tert-butoxycarbonyl)azetidin-2-yl]methoxy}pyridin-4-yl)methyl]amino}-3-[(3-chloro-2-methoxyphenyl)carbamothioyl]-2-oxo-5,6-dihydropyridine-1-carboxylate (1.60 g, 2.32 mmol, 1.00 equiv) and hydrogen peroxide (30 w/w %, 0.34 g, 3.00 mmol, 1.30 equiv) in MeOH (30.0 mL) in portions at room temperature under nitrogen atmosphere. The resulting mixture was stirred for 2 h at 80 degrees C. under nitrogen atmosphere. Desired product could be detected by LCMS. The residue was purified by reverse flash chromatography with the following conditions: column, silica gel; mobile phase, MeCN in water, 10% to 50% gradient in 10 min; detector, UV 254 nm. This resulted in tert-butyl (2S)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]azetidine-1-carboxylate (0.56 g, 36.82%) as a yellow solid.

LC-MS: (M+H)⁺ found 688.2

### 52.5. Synthesis of (S)-2-(3-(azetidin-2-ylmethoxy)pyridin-4-yl)-3-((3-chloro-2-methoxyphenyl)amino)-1,5,6,7-tetrahydro-4H-pyrrolo[3,2-c]pyridin-4-one

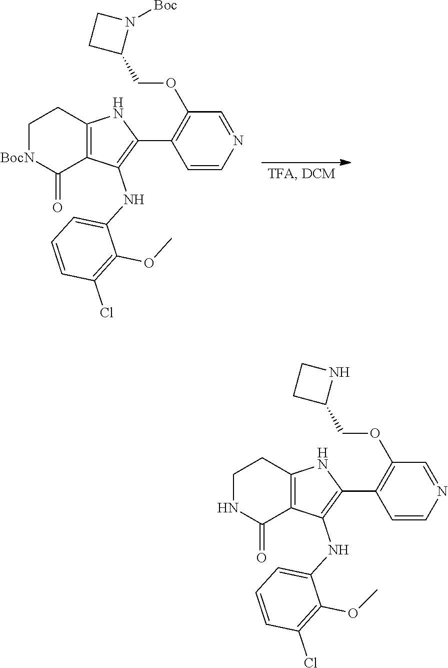

To a stirred mixture of tert-butyl (2S)-2-[({4-[5-(tert-butoxycarbonyl)-3-[(3-chloro-2-methoxyphenyl) amino]-4-oxo-1H,6H,7H-pyrrolo[3,2-c] pyridin-2-yl]pyridin-3-yl}oxy)methyl]azetidine-1-carboxylate (50 mg, 0.08 mmol, 1.00 equiv) and DCM (1.40 mL) was added TFA (1.40 mL) in portions at 0 degrees C. under nitrogen atmosphere. The resulting mixture was stirred for 1 h at room temperature under nitrogen atmosphere. Desired product could be detected by LCMS. The resulting mixture was concentrated under reduced pressure. The crude product was used into next step directly without further purification.

LC-MS: (M+H)⁺ found 454.1

### 52.6. Synthesis of 3-[(3-chloro-2-methoxyphenyl)amino]-2-(3-{[(2S)-1-methanesulfonylazetidin-2-yl]methoxy}pyridin-4-yl)-1H,5H,6H,7H-pyrrolo[3,2-c]pyridin-4-one

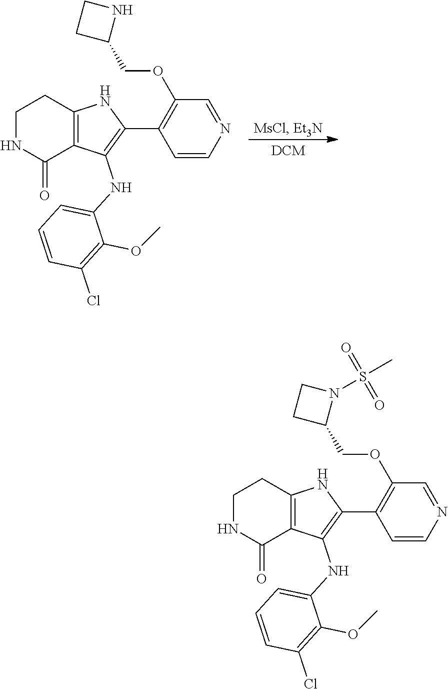

To a stirred mixture of 2-{3-[(2S)-azetidin-2-ylmethoxy]pyridin-4-yl}-3-[(3-chloro-2-methoxyphenyl)amino]-1H,5H,6H,7H-pyrrolo[3,2-c]pyridin-4-one (135 mg, 0.30 mmol, 1.00 equiv) and Et₃N (150 mg, 1.50 mmol, 5.00 equiv) in DCM (2.90 mL) was added MsCl (34 mg, 0.30 mmol, 1.00 equiv) in portions at 0 degrees C. under argon atmosphere. The resulting mixture was stirred for 10 min at 0 degrees C. under argon atmosphere. Desired product could be detected by LCMS. The resulting mixture was concentrated under reduced pressure. The crude product (200 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 20% B to 50% B in 10 min, 50% B; Wave Length: 254/220 nm; RT1(min): 6.32; Number Of Runs: 0) to afford 3-[(3-chloro-2-methoxyphenyl)amino]-2-(3-{[(2S)-1-methanesulfonylazetidin-2-yl]methoxy}pyridin-4-yl)-1H,5H,6H,7H-pyrrolo[3,2-c]pyridin-4-one (27.7 mg, 17.33%) as a white solid.

LC-MS: (M+H)⁺ found 531.9.

¹H NMR (300 MHz, DMSO-d6) δ 10.99 (s, 1H), 8.44 (s, 1H), 8.04 (d, 1H), 7.54 (s, 1H), 7.34 (d, 1H), 7.18 (d, 1H), 6.75-6.62 (m, 2H), 6.21-6.08 (m, 1H), 4.79 (d, 1H), 4.51-4.39 (m, 1H), 4.30-4.17 (m, 1H), 4.07-3.90 (m, 1H), 3.89 (s, 3H), 3.79-3.60 (m, 1H), 3.49-3.32 (m, 2H), 3.13 (s, 3H), 2.90-2.68 (m, 2H), 2.39-2.21 (m, 2H).

### Example 53. 3-[(3-chloro-2-methoxyphenyl)amino]-2-(3-{[(3S)-4-(prop-2-enoyl)morpholin-3-yl]methoxy}pyridin-4-yl)-1H,5H,6H,7H-pyrrolo[3,2-c]pyridin-4-one (compound 521)

### 53.1. Synthesis of tert-butyl (3S)-3-{[(4-cyanopyridin-3-yl)oxy]methyl}morpholine-4-carboxylate

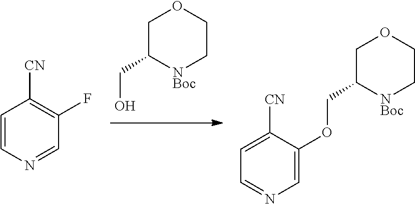

To a stirred solution of 3-fluoropyridine-4-carbonitrile (5.87 g, 27.05 mmol, 1.10 equiv) and tert-butyl (3R)-3-(hydroxymethyl)morpholine-4-carboxylate (3.00 g, 24.59 mmol, 1.00 equiv) in DMF (6 mL) was added Cs₂CO₃ (5.40 g, 16.57 mmol, 1.20 equiv) in portions at room temperature under nitrogen atmosphere. The resulting mixture was stirred for 2h at 60° C. under nitrogen atmosphere. Desired product could be detected in LC-MS. The reaction was diluted with Water (20 mL) at room temperature. The aqueous layer was extracted with EtOAc (3×30 mL) and concentrated under vacuum. The residue was purified by silica gel column chromatography, eluted with PE/EA (1:1) to afford tert-butyl (3S)-3-{[(4-cyanopyridin-3-yl)oxy]methyl}morpholine-4-carboxylate (8.5 g, crude) as off-white solid.

LC-MS: (M+H)⁺ found: 320.00.

### 53.2. Synthesis of tert-butyl (3S)-3-({[4-(aminomethyl)pyridin-3-yl]oxy}methyl)morpholine-4-carboxylate

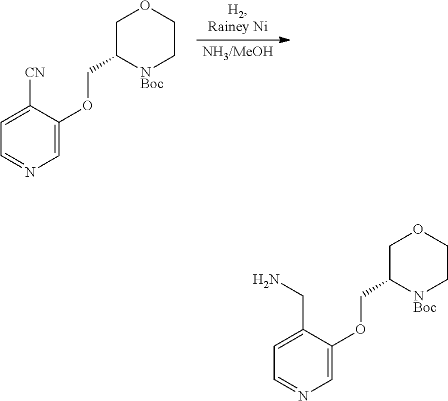 [SMILES: CC(C)(C)OC(=O)N1CCOC[C@H]1COc1cnccc1C#N ; CC(C)(C)OC(=O)N1CCOC[C@H]1COc1cnccc1CN]

To a stirred solution of tert-butyl (3S)-3-{[(4-cyanopyridin-3-yl)oxy]methyl}morpholine-4-carboxylate (8.40 g, 26.30 mmol, 1.00 equiv) and Raney Nickel (4.2 g, 50 w/w %) in MeOH (30 mL) was added Ammonia (7.0 M Solution in MeOH, 15.00 mL, 105.00 mmol) dropwise at room temperature under nitrogen atmosphere. The resulting mixture was stirred for 3h at room temperature under hydrogen atmosphere. Desired product was detected by LCMS. The resulting mixture was filtered; the filter cake was washed with MeOH (3×30 mL). The filtrate was concentrated under reduced pressure to give tert-butyl (3S)-3-({[4-(aminomethyl)pyridin-3-yl]oxy}methyl)morpholine-4-carboxylate (7.6 g, 66.12%) as brown oil.

LC-MS: (M+H)⁺ found: 324.05.

### 53.3. Synthesis of tert-butyl (3S)-3-({[4-({[1-(tert-butoxycarbonyl)-3-[(3-chloro-2-methoxyphenyl)carbamothioyl]-2-oxo-5,6-dihydropyridin-4-yl]amino}methyl) pyridin-3-yl]oxy}methyl)morpholine-4-carboxylate

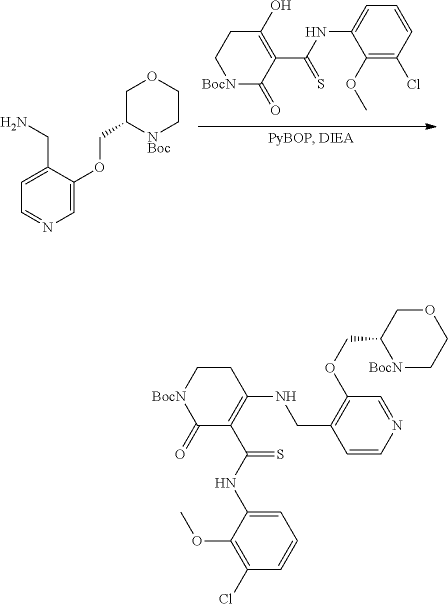 [SMILES: CC(C)(C)OC(=O)N1CCOC[C@H]1COc1cnccc1CN ; COc1c(Cl)cccc1NC(=S)C1=C(O)CCN(C(=O)OC(C)(C)C)C1=O ; COc1c(Cl)cccc1NC(=S)C1=C(NCc2ccncc2OC[C@@H]2COCCN2C(=O)OC(C)(C)C)CCN(C(=O)OC(C)(C)C)C1=O]

To a stirred solution of tert-butyl (3S)-3-({[4-(aminomethyl)pyridin-3-yl]oxy}methyl)morpholine-4-carboxylate (7.50 g, 23.19 mmol, 1.00 equiv) and tert-butyl 3-[(3-chloro-2-methoxyphenyl)carbamothioyl]-4-hydroxy-2-oxo-5,6-dihydropyridine-1-carboxylate (11.49 g, 27.83 mmol, 1.20 equiv) in DMF (70 mL) was added DIEA (8.99 g, 69.58 mmol, 3.00 equiv) and PyBOP (14.48 g, 27.83 mmol, 1.20 equiv) in portions at room temperature under argon atmosphere. The resulting mixture was stirred for 3h at room temperature under argon atmosphere. Desired product could be detected by LCMS. The resulting mixture was concentrated under vacuum. The residue was purified by silica gel column chromatography, eluted with CH₂Cl₂/MeOH (20:1) to afford tert-butyl (3S)-3-({[4-({[1-(tert-butoxycarbonyl)-3-[(3-chloro-2-methoxyphenyl)carbamothioyl]-2-oxo-5,6-dihydropyridin-4-yl]amino}methyl) pyridin -3-yl]oxy}methyl)morpholine-4-carboxylate (9.60 g, 43.22%) as a yellow green oil.

LC-MS: (M+H)⁺ found: 718.2.

### 53.4. Synthesis of tert-butyl (3S)-3-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy) methyl]morpholine-4-carboxylate

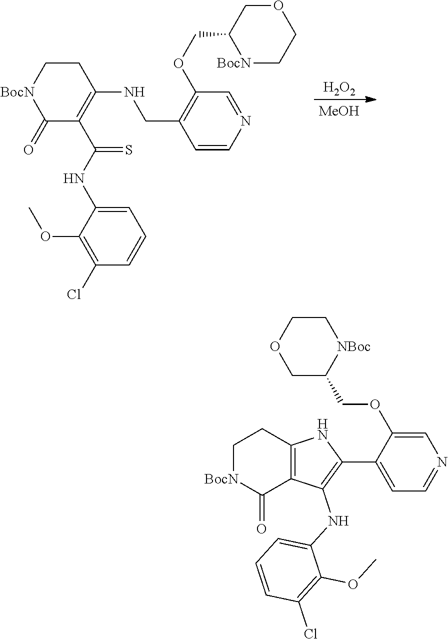 [SMILES: "MeOH" ; COc1c(Cl)cccc1NC(=S)C1=C(NCc2ccncc2OC[C@@H]2COCCN2C(=O)OC(C)(C)C)CCN(C(=O)OC(C)(C)C)C1=O ; "H2O2" ; COc1c(Cl)cccc1Nc1c(-c2ccncc2OC[C@@H]2COCCN2C(=O)OC(C)(C)C)[nH]c2c1C(=O)N(C(=O)OC(C)(C)C)CC2]

To a stirred solution of tert-butyl (3S)-3-({[4-({[1-(tert-butoxycarbonyl)-3-[(3-chloro-2-methoxyphenyl) carbamothioyl]-2-oxo-5,6-dihydropyridin-4-yl]amino}methyl)pyridin-3-yl]oxy}methyl)morpholine-4-carboxylate (9.00 g, 12.55 mmol, 1.00 equiv) in MeOH (90 mL) was added H₂O₂ (30%) (2.13 g, 18.80 mmol, 1.50 equiv) dropwise at room temperature under nitrogen atmosphere. The resulting mixture was stirred for 2h at 80° C. under nitrogen atmosphere. Desired product could be detected by LCMS. The resulting mixture was concentrated under vacuum. The residue was purified by silica gel column chromatography, eluted with CH2Cl2/MeOH (10:1) to afford tert-butyl (3S)-3-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy) methyl]morpholine-4-carboxylate (8.00 g, 55.99%) as Brown yellow oil.

LC-MS: (M+H)+ found 684.1.

### 53.5. Synthesis of (S)-3-((3-chloro-2-methoxyphenyl)amino)-2-(3-(morpholin-3-ylmethoxy)pyridin-4-yl)-1,5,6,7-tetrahydro-4H-pyrrolo[3,2-c]pyridin-4-one

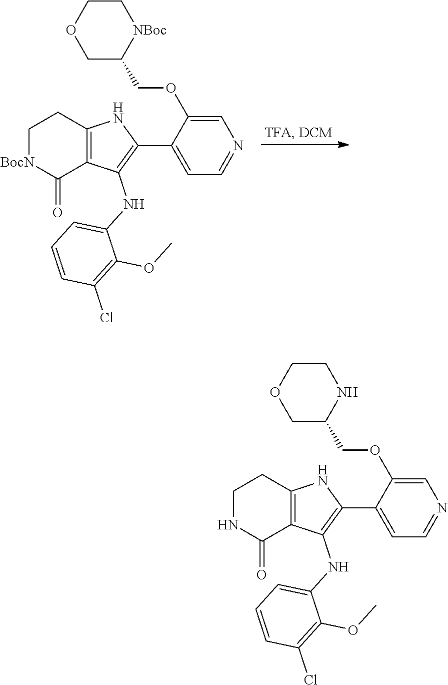

To a stirred solution of tert-butyl (3S)-3-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}morpholine-4-carboxylate (100 mg, 0.17 mmol, 1.00 equiv) in DCM (3 mL) was added TFA (2 mL) dropwise at room temperature. The resulting mixture was stirred for 1 h at room temperature. Desired product could be detected by LCMS. The resulting mixture was concentrated under vacuum. The residue was used directly in next step without any further purification.

LC-MS: (M+H)⁺ found: 484.05.

### 53.6. Synthesis of 3-[(3-chloro-2-methoxyphenyl)amino]-2-(3-{[(3S)-4-(prop-2-enoyl)morpholin-3-yl]methoxy}pyridin-4-yl)-1H,5H,6H,7H-pyrrolo[3,2-c]pyridin-4-one

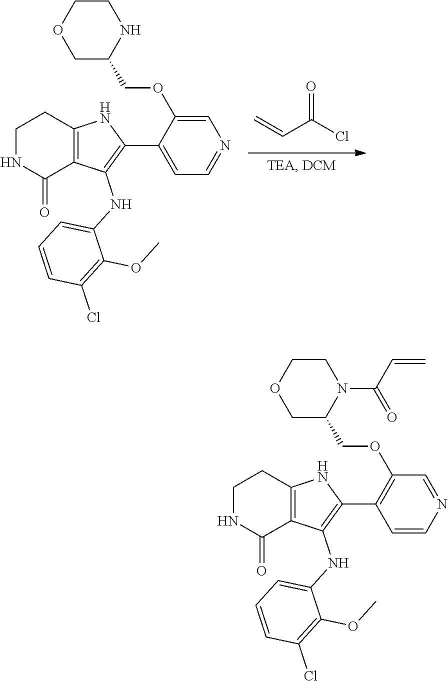

To a solution of 3-[(3-chloro-2-methoxyphenyl) amino]-2-{3-[(3S)-morpholin-3-ylmethoxy]pyridin-4-yl}-1H,5H, 6H,7H-pyrrolo[3,2-c]pyridin-4-one (100 mg, 0.21 mmol, 1.00 equiv) in DCM (3.00 mL) was added TEA (209 mg, 2.07 mmol, 10.00 equiv) and acryloyl chloride (13 mg, 0.15 mmol, 0.70 equiv) dropwise at 0° C. The resulting mixture was stirred for 1 h at 0° C. under nitrogen atmosphere. The reaction was quenched with MeOH at 0° C. The resulting mixture was concentrated under vacuum. The crude product (150 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 22% B to 55% B in 7 min; Wave Length: 254 nm; RT1(min): 6.5; Number Of Runs: 0) to afford 3-[(3-chloro-2-methoxyphenyl)amino]-2-(3-{[(3S)-4-(prop-2-enoyl)morpholin-3-yl]methoxy}pyridin-4-yl)-1H,5H,6H,7H-pyrrolo[3,2-c]pyridin-4-one (9.0 mg, 7.89%) as white solid.

LC-MS: (M+H)⁺ found 537.95.

¹H NMR (300 MHz, DMSO-d6) δ 11.01 (s, 1H), 8.46 (s, 1H), 8.11-7.93 (m, 1H), 7.61-7.04 (m, 3H), 6.85 (m, 1H), 6.75-6.56 (m, 2H), 6.26-5.94 (m, 2H), 5.82-5.39 (m, 1H), 5.10-4.70 (m, 1H), 4.55-4.11 (m, 2H), 4.08-3.72 (m, 6H), 3.71-3.33 (m, 5H), 3.11-2.72 (m, 2H).

### Example 54. 3-[(3-chloro-2-methoxyphenyl)amino]-2-(3-{[(2R)-1-[(2E)-4-(dimethylamino)but-2-enoyl]azetidin-2-yl] methoxy}pyridin-4-yl)-1H,5H,6H,7H-pyrrolo[3,2-c] pyridin-4-one (compound 199)

### 54.1. Synthesis of (E)-4-(dimethylamino)but-2-enoyl chloride

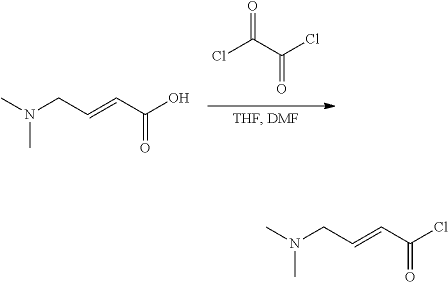

To a stirred solution of (2E)-4-(dimethylamino) but-2-enoic acid (100 mg, 0.78 mmol, 1.00 equiv) in THF (4 mL) was added oxalyl chloride (108 mg, 0.85 mmol, 1.10 equiv) dropwise and DMF (3 mg, 0.04 mmol, 0.05 equiv) at 0° C. under nitrogen atmosphere. The resulting mixture was stirred for 1 h at 0° C. under nitrogen atmosphere. Desired product could be detected by TLC. The reaction was used directly in next step without work-up.

### 54.2. Synthesis of 3-[(3-chloro-2-methoxyphenyl)amino]-2-(3-{[(2R)-1-[(2E)-4-(dimethylamino)but-2-enoyl]azetidin-2-yl] methoxy}pyridin-4-yl)-1H,5H,6H,7H-pyrrolo[3,2-c] pyridin-4-one

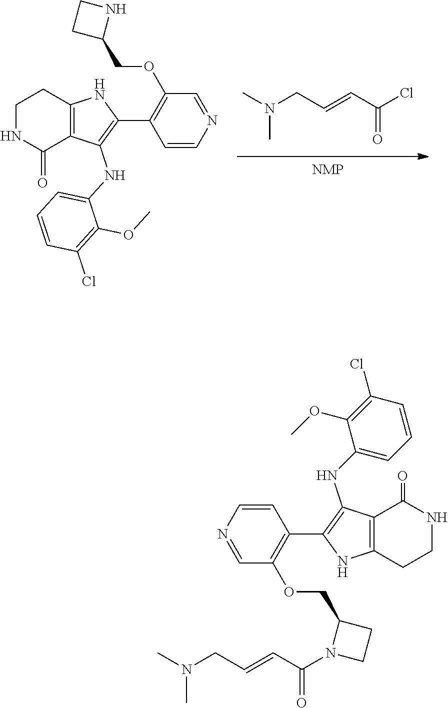

To a stirred solution of 2-{3-[(2R)-azetidin-2-ylmethoxy] pyridin-4-yl}-3-[(3-chloro-2-methoxyphenyl) amino]-1H,5H,6H,7H-pyrrolo[3,2-c] pyridin-4-one (80 mg, 0.18 mmol, 1.00 equiv) in NMP (4 mL) was added (2E)-4-(dimethylamino) but-2-enoyl chloride (78 mg, 0.53 mmol, 3.00 equiv) dropwise at 0° C. under argon atmosphere. The resulting mixture was stirred for 1 h at 0° C. under argon atmosphere. Desired product could be detected in LC-MS. The reaction was quenched by the addition of MeOH (5 mL) at 0° C. The resulting mixture was concentrated under vacuum. The crude product (40 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 21% B to 51% B in 7 min; Wave Length: 254 nm; RT1(min): 6.5; Number Of Runs: 0) to afford 3-[(3-chloro-2-methoxyphenyl)amino]-2-(3-{[(2R)-1-[(2E)-4-(dimethylamino)but-2-enoyl]azetidin-2-yl]methoxy}pyridin-4-yl)-1H,5H,6H,7H-pyrrolo[3,2-c] pyridin-4-one (8.3 mg, 8.33%) as off-white solid.

LC-MS: (M+H)⁺ found 565.35

¹H NMR (400 MHz, DMSO-d6) δ 11.89 (s, 1H), 8.39 (s, 1H), 7.98 (d, 1H), 7.56 (s, 1H), 7.36 (d, 1H), 7.15 (t, 1H), 6.77-6.63 (m, 3H), 6.19-6.08 (m, 2H), 4.85 (q, 1H), 4.56-4.51 (m, 1H), 4.41-4.38 (m, 1H), 4.23-4.17 (m, 2H), 3.91 (s, 3H), 3.45-3.41 (m, 2H), 3.12-2.90 (m, 4H), 3.33-2.96 (m, 3H), 2.52-1.94 (m, 5H).

### Example 55. 3-[(3-fluoro-2-methylphenyl)amino]-2-(3-{[(2S)-1-(2-fluoroprop-2-enoyl)pyrrolidin-2-yl]methoxy}pyridin-4-yl)-1H,5H,6H,7H-pyrrolo[3,2-c]pyridin-4-one (compound 218)

### 55.1. Synthesis of 3-[(3-fluoro-2-methylphenyl)amino]-2-{3-[(2S)-pyrrolidin-2-ylmethoxy]pyridin-4-yl}-1H,5H,6H,7H-pyrrolo[3,2-c]pyridin-4-one

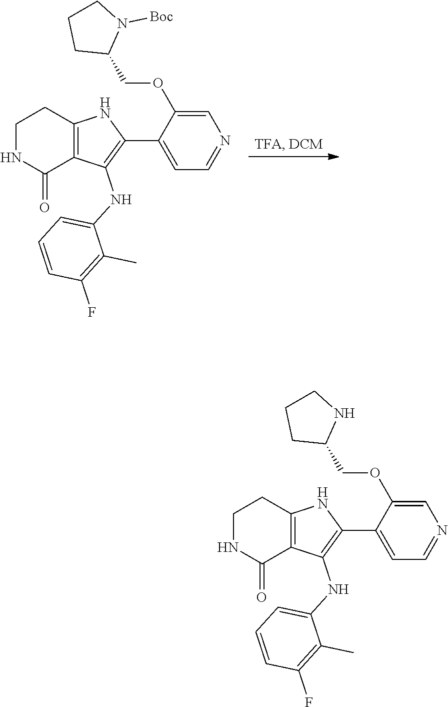 [SMILES: Cc1c(F)cccc1Nc1c(-c2ccncc2OC[C@@H]2CCCN2C(=O)OC(C)(C)C)[nH]c2c1C(=O)NCC2 ; Cc1c(F)cccc1Nc1c(-c2ccncc2OC[C@@H]2CCCN2)[nH]c2c1C(=O)NCC2]

To a stirred solution of tert-butyl (2S)-2-{[(4-{3-[(3-fluoro-2-methylphenyl)amino]-4-oxo-1H,5H,6H,7H-pyrrolo[3,2-c]pyridin-2-yl}pyridin-3-yl)oxy]methyl}pyrrolidine-1-carboxylate (40 mg, 0.075 mmol, 1 equiv) in DCM (1 mL) was added TFA (0.3 mL) at room temperature under nitrogen atmosphere. The resulting mixture was stirred for 1 h and concentrated under reduced pressure to afford 3-[(3-fluoro-2-methylphenyl)amino]-2-{3-[(2S)-pyrrolidin-2-ylmethoxy]pyridin-4-yl}-1H,5H,6H,7H-pyrrolo[3,2-c]pyridin-4-one (32 mg, crude) as a reddish brown yellow oil.

LC-MS: (M+H)⁺ found 436.0

### 55.2. Synthesis of 3-[(3-fluoro-2-methylphenyl)amino]-2-(3-{[(2S)-1-(2-fluoroprop-2-enoyl)pyrrolidin-2-yl]methoxy}pyridin-4-yl)-1H,5H,6H,7H-pyrrolo[3,2-c]pyridin-4-one

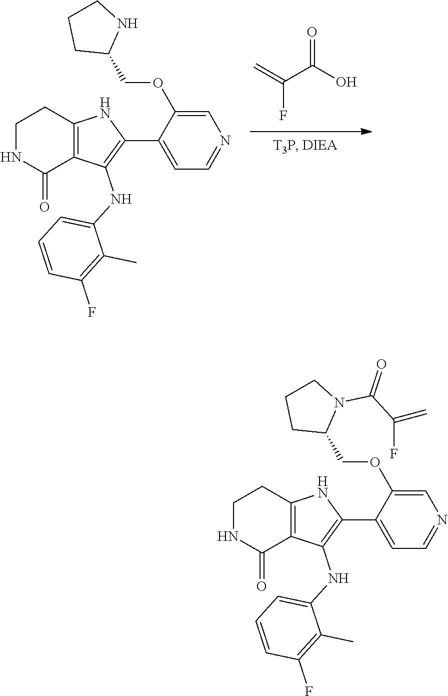

To a stirred solution of 3-[(3-fluoro-2-methylphenyl)amino]-2-{3-[(2S)-pyrrolidin-2-ylmethoxy]pyridin-4-yl}-1H,5H,6H,7H-pyrrolo[3,2-c]pyridin-4-one (32 mg, 0.073 mmol, 1 equiv) in THF (3 mL) was basified to pH 8 with DIEA. To the above mixture was added 2-fluoroprop-2-enoic acid (9.9 mg, 0.109 mmol, 1.5 equiv) at 0° C. under nitrogen atmosphere followed by the addition of T3P (46.8 mg, 0.145 mmol, 2.0 equiv, 50% in EA) dropwise. The resulting mixture was stirred for 1 h at room temperature under nitrogen atmosphere. Saturated aqueous NaHCO₃ (10 mL) was added to the reaction mixture at 0° C. and extracted with EtOAc (3×10 mL), dried over anhydrous Na₂SO₄ and filtered. The filtrate was concentrated under reduced pressure to give crude product (40 mg) that was purified by Prep-HPLC under the following conditions (YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 30% B to 54% B in 9 min, 54% B; Wave Length: 254/220 nm; RT1(min): 8.85; Number Of Runs: 0) to give 3-[(3-fluoro-2-methylphenyl)amino]-2-(3-{[(2S)-1-(2-fluoroprop-2-enoyl)pyrrolidin-2-yl]methoxy}pyridin-4-yl)-1H,5H,6H,7H-pyrrolo[3,2-c]pyridin-4-one (10.3 mg, 27.6%) as a light-yellow solid.

LC-MS: (M+H)⁺ found 508.15

¹H NMR (400 MHz, Chloroform-d) δ 11.16 (s, 1H), 8.21 (s, 1H), 7.91 (d, 1H), 7.44 (s, 1H), 7.31 (d, 1H), 6.76-6.71 (m, 1H), 6.50 (t, 1H), 6.17 (d, 1H), 5.57-5.44 (m, 1H), 5.33-5.18 (m, 2H), 5.06 (s, 1H), 4.27 (t, 1H), 4.17-4.14 (m, 1H), 3.92-3.77 (m, 2H), 3.75-3.55 (m, 2H), 3.14 (t, 2H), 2.34 (s, 3H), 2.29-2.01 (m, 3H), 1.85-1.78 (m, 1H).

### Example 56. 3-[(3-chloro-2-methoxyphenyl)amino]-2-(3-{2-[(2R)-1-[(2E)-4-(dimethylamino)but-2-enoyl]pyrrolidin-2-yl]ethynyl}pyridin-4-yl)-1H,5H,6H,7H-pyrrolo[3,2-c]pyridin-4-one (compound 523)

### 56.1. Synthesis of 3-[(3-chloro-2-methoxyphenyl) amino]-2-(3-{2-[(2R)-pyrrolidin-2-yl] ethynyl} pyridin-4-yl)-1H,5H,6H,7H-pyrrolo[3,2-c] pyridin-4-one

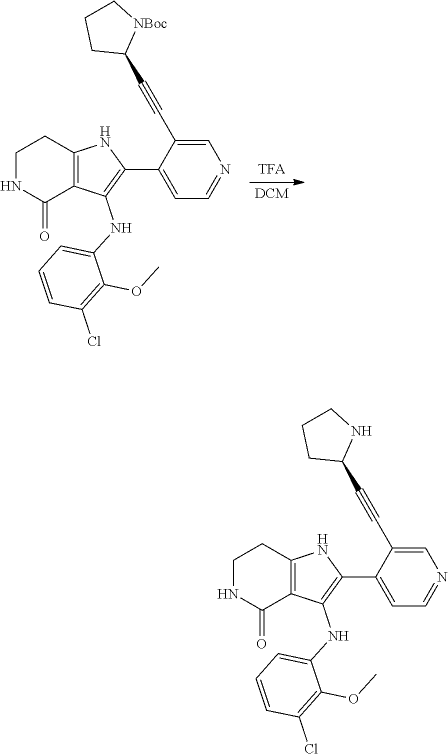

A solution of tert-butyl (2R)-2-[2-(4-{3-[(3-chloro-2-methoxyphenyl) amino]-4-oxo-1H,5H,6H,7H-pyrrolo[3,2-c] pyridin-2-yl} pyridin-3-yl) ethynyl] pyrrolidine-1-carboxylate (100 mg, 0.178 mmol, 1 equiv) in DCM (2 mL) was added TFA (0.7 mL) at room temperature under nitrogen atmosphere. The resulting mixture was stirred for 1 h and concentrated under reduced pressure to afford 3-[(3-chloro-2-methoxyphenyl) amino]-2-(3-{2-[(2R)-pyrrolidin-2-yl] ethynyl} pyridin-4-yl)-1H,5H,6H,7H-pyrrolo[3,2-c] pyridin-4-one (100 mg, crude) as a red oil. LCMS: (M+H)⁺ found 462.

### 56.2. Synthesis of the (2E)-4-(dimethylamino) but-2-enoyl chloride

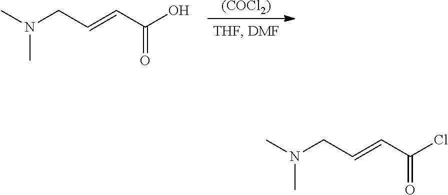

To a stirred solution of (2E)-4-(dimethylamino) but-2-enoic acid (100 mg, 0.774 mmol, 1 equiv) in THF (6 mL) was added (COCl)₂ (108 mg, 0.851 mmol, 1.1 equiv) and DMF (0.1 mL) at 0° C. under nitrogen atmosphere. The resulting mixture was stirred for 1 h at 0° C. under nitrogen atmosphere. TLC (PE/EA=2:1) showed a new spot was detected. The resulting mixture was concentrated under reduced pressure to give the (2E)-4-(dimethylamino) but-2-enoyl chloride (80 mg, crude) as brown oil.

### 56.3. Synthesis of 3-[(3-chloro-2-methoxyphenyl)amino]-2-(3-{2-[(2R)-1-[(2E)-4-(dimethylamino)but-2-enoyl]pyrrolidin-2-yl]ethynyl}pyridin-4-yl)-1H,5H,6H,7H-pyrrolo[3,2-c]pyridin-4-one

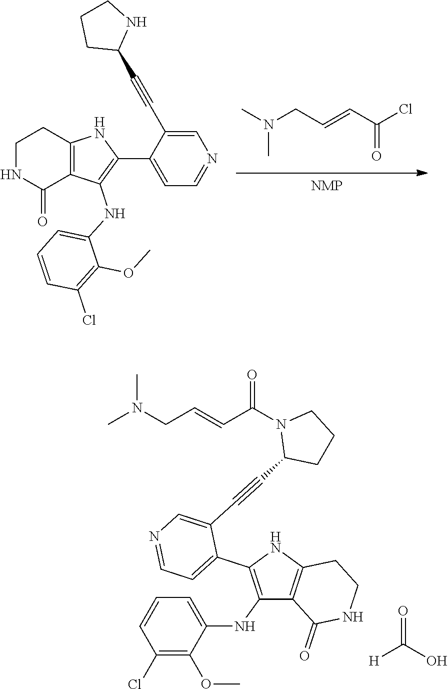

A mixture of 3-[(3-chloro-2-methoxyphenyl) amino]-2-(3-{2-[(2R)-pyrrolidin-2-yl]ethynyl} pyridin-4-yl)-1H,5H,6H,7H-pyrrolo[3,2-c] pyridin-4-one (100 mg, 0.216 mmol, 1.00 equiv) in NMP (2 mL) was basified to pH 8 with DIEA. To the above mixture was added (2E)-4-(dimethylamino) but-2-enoyl chloride (47.9 mg, 0.324 mmol, 1.5 equiv) dropwise at 0° C. under argon atmosphere. The resulting mixture was stirred for 30 min at 0° C. The resulting mixture was concentrated under reduced pressure and purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 9% B to 25% B in 10 min, 25% B; Wave Length: 254/220 nm) to afford 3-[(3-chloro-2-methoxyphenyl)amino]-2-(3-{2-[(2R)-1-[(2E)-4-(dimethylamino)but-2-enoyl]pyrrolidin-2-yl]ethynyl}pyridin-4-yl)-1H,5H,6H,7H-pyrrolo[3,2-c]pyridin-4-one (31.7 mg, 25.3%) as a light brown solid.

LCMS: (M+H)⁺ found 573.35.

¹H NMR (300 MHz, Chloroform-d) δ 11.09 (s, 1H), 8.54 (s, 1H), 8.19-8.10 (m, 1H), 7.69 (s, 1H), 7.42 (d, 1H), 6.93-6.73 (m, 1H), 6.71-6.65 (m, 1H), 6.60 (t, 1H), 6.51-6.42 (m, 1H), 6.25-6.12 (m, 1H), 5.58 (s, 1H), 4.89 (t, 1H), 4.06 (s, 3H), 3.88-3.74 (m, 1H), 3.72-3.31 (m, 5H), 3.29-3.20 (m, 2H), 2.49 (s, 6H), 2.39-2.21 (m, 3H), 2.15-2.05 (m, 1H).

### Example 57. 2-(3-{2-[1-(difluoromethyl)cyclopropyl]ethynyl}pyridin-4-yl)-3-[(3-fluoro-2-methoxyphenyl)amino]-1H,5H,6H,7H-pyrrolo[3,2-c]pyridin-4-one (compound 558)

### 57.1. Synthesis of 3-[(3-fluoro-2-methoxyphenyl) amino]-2-(3-iodopyridin-4-yl)-1H,5H,6H,7H-pyrrolo[3,2-c] pyridin-4-one

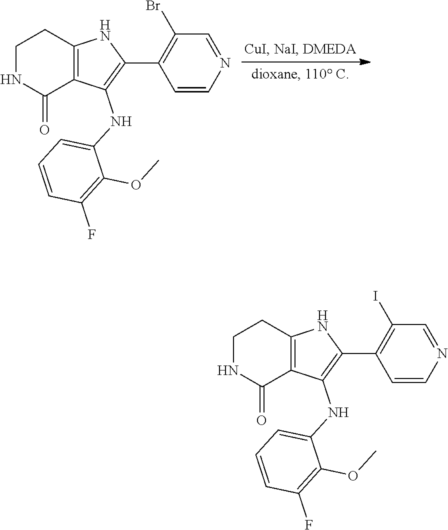 [SMILES: COc1c(F)cccc1Nc1c(-c2ccncc2Br)[nH]c2c1C(=O)NCC2 ; COc1c(F)cccc1Nc1c(-c2ccncc2I)[nH]c2c1C(=O)NCC2]

To a stirred mixture of 2-(3-bromopyridin-4-yl)-3-[(3-fluoro-2-methoxyphenyl) amino]-1H,5H,6H,7H-pyrrolo[3,2-c] pyridin-4-one (500 mg, 1.16 mmol, 1.00 equiv), CuI (22 mg, 0.116 mmol, 0.10 equiv) and NaI (347 mg, 2.32 mmol, 2.00 equiv) in dioxane (5 mL) were added DMEDA (51 mg, 0.580 mmol, 0.50 equiv) dropwise. The resulting mixture was stirred for overnight at 110 degrees C. under argon atmosphere. Desired product could be detected by LCMS. The resulting mixture was concentrated under reduced pressure. The residue was purified by silica gel column chromatography, eluted with CH2Cl2/MeOH (20:1) to afford 3-[(3-fluoro-2-methoxyphenyl) amino]-2-(3-iodopyridin-4-yl)-1H,5H,6H,7H-pyrrolo[3,2-c] pyridin-4-one (320 mg, 57.71%) as a yellow solid.

LC-MS: (M+H)⁺ found 478.95.

### 57.2. Synthesis of 2-(3-{2-[1-(difluoromethyl)cyclopropyl]ethynyl}pyridin-4-yl)-3-[(3-fluoro-2-methoxyphenyl)amino]-1H,5H,6H,7H-pyrrolo[3,2-c]pyridin-4-one

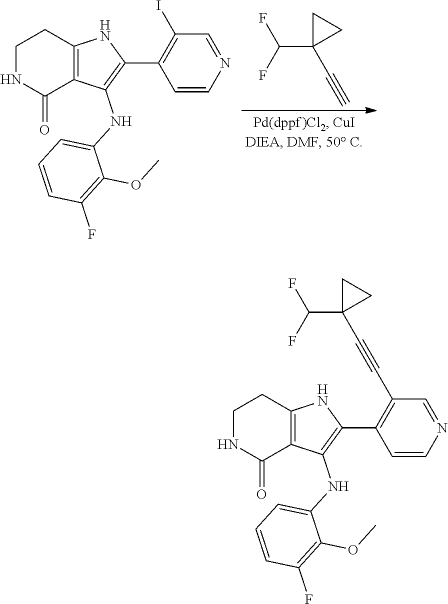

To a stirred mixture of 3-[(3-chloro-2-methoxyphenyl)amino]-2-(3-iodopyridin-4-yl)-1H,5H,6H,7H-pyrrolo[3,2-c]pyridin-4-one (80 mg, 0.162 mmol, 1.00 equiv), CuI (4 mg, 0.02 mmol, 0.10 equiv), Pd(dppf)Cl₂·CH₂Cl₂ (17 mg, 0.02 mmol, 0.10 equiv) and DIEA (108 mg, 0.836 mmol, 4.00 equiv) in DMF (1 mL) were added 1-(difluoromethyl)-1-ethynylcyclopropane (48 mg, 0.418 mmol, 2 equiv) under argon atmosphere. The resulting mixture was stirred for 4 h at 50 degrees C. under argon atmosphere. Desired product could be detected by LCMS. The resulting mixture was concentrated under reduced pressure. The residue was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 28% B to 47% B in 8 min, 47% B; Wave Length: 254/220 nm; RT1(min): 8; Number Of Runs: 0) to afford 2-(3-{2-[1-(difluoromethyl)cyclopropyl]ethynyl}pyridin-4-yl)-3-[(3-fluoro-2-methoxyphenyl)amino]-1H,5H,6H,7H-pyrrolo[3,2-c]pyridin-4-one (42.9 mg, 43.33%) as a yellow solid.

LC-MS: (M+H)⁺ found 466.95.

¹H NMR (400 MHz, DMSO-d6) δ 11.40 (s, 1H), 8.86-8.21 (m, 2H), 7.42-7.29 (m, 2H), 7.13 (t, 1H), 6.61-6.54 (m, 1H), 6.46-6.39 (m, 1H), 5.98-5.67 (m, 2H), 3.87 (s, 3H), 3.47-3.41 (m, 2H), 2.82 (t, 2H), 1.32-1.18 (m, 4H).

### Example 58. 2-(3-{2-[1-(difluoromethyl)cyclopropyl]ethynyl}pyridin-4-yl)-3-[(3-fluoro-2-methylphenyl)amino]-1H,5H,6H,7H-pyrrolo[3,2-c]pyridin-4-one (compound 557)

### 58.1. Synthesis of 3-[(3-fluoro-2-methylphenyl) amino]-2-(3-iodopyridin-4-yl)-1H,5H,6H,7H-pyrrolo[3,2-c] pyridin-4-one

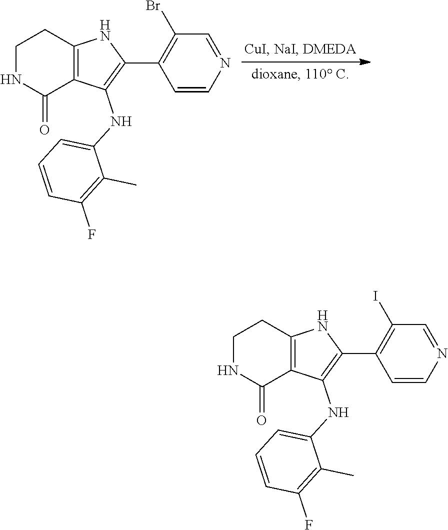 [SMILES: Cc1c(F)cccc1Nc1c(-c2ccncc2Br)[nH]c2c1C(=O)NCC2 ; Cc1c(F)cccc1Nc1c(-c2ccncc2I)[nH]c2c1C(=O)NCC2]

To a stirred mixture of 2-(3-bromopyridin-4-yl)-3-[(3-fluoro-2-methylphenyl) mamino]-1H,5H,6H,7H-pyrrolo[3,2-c] pyridin-4-one (500 mg, 1.20 mmol, 1.00 equiv), CuI (23 mg, 0.12 mmol, 0.10 equiv) and NaI (361 mg, 2.40 mmol, 2.00 equiv) in dioxane (5 mL) were added DMEDA (53 mg, 0.602 mmol, 0.50 equiv) dropwise. The resulting mixture was stirred for overnight at 110 degrees C. under argon atmosphere. LCMS confirmed completion of reaction and desired product was observed. The resulting mixture was concentrated under reduced pressure. The residue was purified by silica gel column chromatography, eluted with CH₂Cl₂/MeOH (20:1) to afford 3-[(3-fluoro-2-methylphenyl) amino]-2-(3-iodopyridin-4-yl)-1H,5H,6H,7H-pyrrolo[3,2-c] pyridin-4-one (300 mg, 53.90%) as a yellow solid.

LC-MS: (M+H)⁺ found 463.0.

### 58.2. Synthesis of 2-(3-{2-[1-(difluoromethyl)cyclopropyl]ethynyl}pyridin-4-yl)-3-[(3-fluoro-2-methylphenyl)amino]-1H,5H,6H,7H-pyrrolo[3,2-c]pyridin-4-one

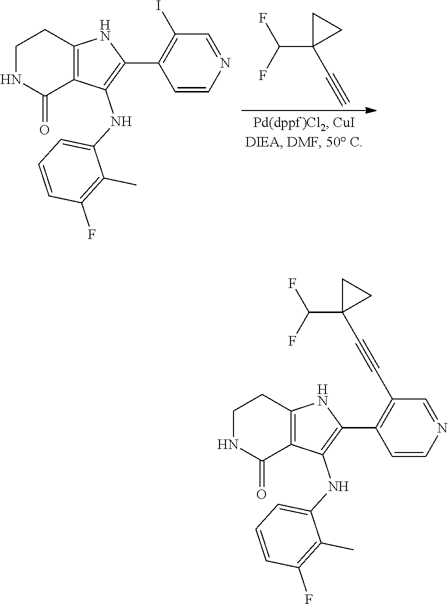

To a stirred mixture of 3-[(3-chloro-2-methoxyphenyl)amino]-2-(3-iodopyridin-4-yl)-1H,5H,6H,7H-pyrrolo[3,2-c]pyridin-4-one (80 mg, 0.162 mmol, 1.00 equiv), CuI (3 mg, 0.02 mmol, 0.10 equiv), Pd(dppf)Cl₂·CH₂Cl₂ (14 mg, 0.02 mmol, 0.10 equiv) and DIEA (89 mg, 0.692 mmol, 4.0 equiv) in DMF (1 mL) were added 1-(difluoromethyl)-1-ethynylcyclopropane (40 mg, 0.346 mmol, 2.0 equiv) under argon atmosphere. The resulting mixture was stirred for 4 h at 50 degrees C. under argon atmosphere. Desired product could be detected by LCMS. The resulting mixture was concentrated under reduced pressure. The residue was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 28% B to 47% B in 8 min, 47% B; Wave Length: 254/220 nm; RT1(min): 8; Number Of Runs: 0) to afford 2-(3-{2-[1-(difluoromethyl)cyclopropyl]ethynyl}pyridin-4-yl)-3-[(3-fluoro-2-methylphenyl)amino]-1H,5H,6H,7H-pyrrolo[3,2-c]pyridin-4-one (14.7 mg, 18.67%) as a yellow solid.

LC-MS: (M+H)⁺ found 451.00.

¹H NMR (400 MHz, DMSO-d6) δ 11.32 (s, 1H), 8.53 (s, 1H), 8.31 (d, 1H), 7.23 (d, 1H), 7.20-7.14 (m, 2H), 6.74-6.67 (m, 1H), 6.40 (t, 1H), 6.02 (d, 1H), 5.98-5.65 (m, 1H), 3.47-3.40 (m, 2H), 2.83 (t, 2H), 2.14 (s, 3H), 1.29-1.17 (m, 4H).

### Example 59. 2-(3-{[(2S)-1-[(2E)-4-(dimethylamino) but-2-enoyl] pyrrolidin-2-yl]methoxy} pyridin-4-yl)-3-[(3-fluoro-2-methylphenyl) amino]-1H,5H,6H,7H-pyrrolo[3,2-c] pyridin-4-one (compound 217)

### 59.1. Synthesis of 3-[(3-fluoro-2-methylphenyl) amino]-2-{3-[(2S)-pyrrolidin-2-ylmethoxy] pyridin-4-yl}-1H,5H,6H,7H-pyrrolo[3,2-c] pyridin-4-one

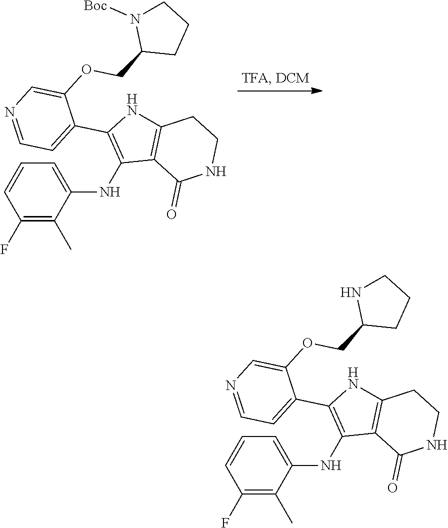 [SMILES: Cc1c(F)cccc1Nc1c(-c2ccncc2OC[C@@H]2CCCN2C(=O)OC(C)(C)C)[nH]c2c1C(=O)NCC2 ; Cc1c(F)cccc1Nc1c(-c2ccncc2OC[C@@H]2CCCN2)[nH]c2c1C(=O)NCC2]

A solution of tert-butyl (2S)-2-{[(4-{3-[(3-fluoro-2-methylphenyl) amino]-4-oxo-1H,5H,6H,7H-pyrrolo[3,2-c] pyridin-2-yl} pyridin-3-yl) oxy] methyl} pyrrolidine-1-carboxylate (40 mg, 0.075 mmol, 1 equiv) in DCM (1 mL) was added TFA (0.3 mL) at room temperature under nitrogen atmosphere. The resulting mixture was stirred for 1 h and concentrated under reduced pressure to afford 3-[(3-fluoro-2-methylphenyl) amino]-2-{3-[(2S)-pyrrolidin-2-ylmethoxy] pyridin-4-yl}-1H,5H,6H,7H-pyrrolo[3,2-c] pyridin-4-one (32 mg, crude) as a light yellow solid.

LC-MS: (M+H)⁺ found 436.0

### 59.2. Synthesis of 2-(3-{[(2S)-1-[(2E)-4-(dimethylamino) but-2-enoyl] pyrrolidin-2-yl] methoxy} pyridin-4-yl)-3-[(3-fluoro-2-methylphenyl) amino]-1H,5H,6H,7H-pyrrolo[3,2-c] pyridin-4-one

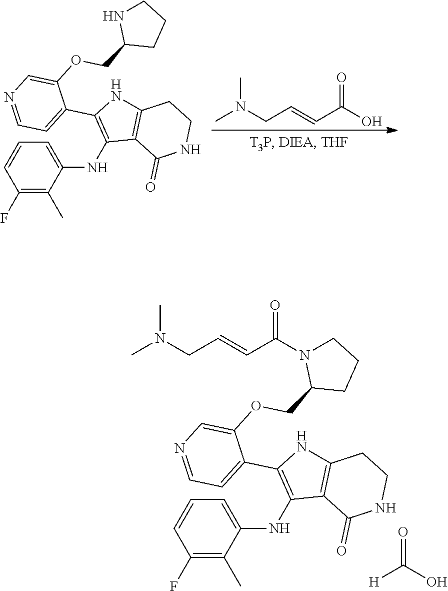

To a stirred solution of 3-[(3-fluoro-2-methylphenyl) amino]-2-{3-[(2S)-pyrrolidin-2-ylmethoxy] pyridin-4-yl}-1H,5H,6H,7H-pyrrolo[3,2-c] pyridin-4-one (32 mg, 0.073 mmol, 1 equiv) in THF (1 mL) was basified to pH 8 with DIEA. To the above mixture was added (2E)-4-(dimethylamino) but-2-enoic acid (14.2 mg, 0.11 mmol, 1.50 equiv) at 0° C. under nitrogen atmosphere followed by the addition of T3P (46.7 mg, 0.146 mmol, 2.00 equiv, 50% in EA) dropwise. The resulting mixture was stirred for 1 h at room temperature under nitrogen atmosphere. Saturated aqueous NaHCO₃ (10 mL) was added to the reaction mixture at 0° C. and extracted with EtOAc (3×10 mL), dried over anhydrous Na₂SO₄ and filtered. The filtrate was concentrated under reduced pressure to give crude product (60 mg) that was purified by Prep-HPLC under the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 9% B to 19% B in 7 min, 19% B; Wave Length: 254/220 nm) to afford 2-(3-{[(2S)-1-[(2E)-4-(dimethylamino) but-2-enoyl] pyrrolidin-2-yl] methoxy} pyridin-4-yl)-3-[(3-fluoro-2-methylphenyl) amino]-1H,5H,6H,7H-pyrrolo[3,2-c] pyridin-4-one (10.9 mg, 26.8%) as a yellow solid.

LC-MS: (M+H)*found 547.15

¹H NMR (300 MHz, Chloroform-d) δ 11.49 (s, 1H), 8.20 (s, 1H), 7.91 (d, 1H), 7.38-7.31 (m, 2H), 6.94-6.80 (m, 1H), 6.78-6.63 (m, 1H), 6.55-6.42 (m, 2H), 6.17 (d, 1H), 5.47 (s, 1H), 5.11-4.93 (m, 1H), 4.26 (t, 1H), 4.09-3.85 (m, 1H), 3.76 (t, 2H), 3.69-3.54 (m, 2H), 3.37-3.26 (m, 2H), 3.24-3.16 (m, 2H), 2.42 (s, 6H), 2.34 (s, 3H), 2.21-2.05 (m, 3H), 1.96-1.84 (m, 1H).

### Example 60. 3-[(3-chloro-2-ethylphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (compound 216)

### 60.1. Synthesis of tert-butyl (2S)-2-{[(4-{3-[(3-chloro-2-ethylphenyl) amino]-4-oxo-1H,5H,6H,7H-pyrrolo[3,2-c] pyridin-2-yl} pyridin-3-yl) oxy] methyl} pyrrolidine-1-carboxylate

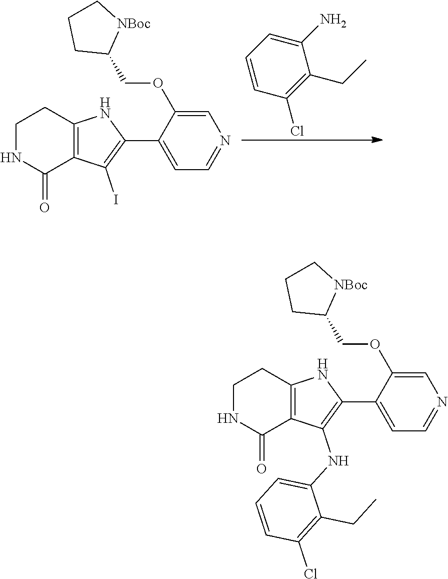

To a stirred solution of tert-butyl (2S)-2-{[(4-{3-iodo-4-oxo-1H,5H,6H,7H-pyrrolo[3,2-c]pyridin-2-yl} pyridin-3-yl) oxy] methyl} pyrrolidine-1-carboxylate (90 mg, 0.167 mmol, 1 equiv) and Cs₂CO₃ (108 mg, 0.334 mmol, 2 equiv) in DMF (1 mL) were added EPhos Pd G4 (15.3 mg, 0.017 mmol, 0.1 equiv) and 3-chloro-2-ethylaniline (26 mg, 0.167 mmol, 1 equiv) at room temperature under argon atmosphere. The resulting mixture was stirred for 2 h at 50° C. under argon atmosphere. The resulting mixture was concentrated under reduced pressure. The residue was purified by silica gel column chromatography, eluted with CH₂Cl₂/MeOH (10:1) to afford tert-butyl (2S)-2-{[(4-{3-[(3-chloro-2-ethylphenyl) amino]-4-oxo-1H,5H,6H,7H-pyrrolo[3,2-c] pyridin-2-yl} pyridin-3-yl) oxy]methyl} pyrrolidine-1-carboxylate (90 mg, 95.1%) as a yellow solid.

LC-MS: M+H found: 566.0.

### 60.2. Synthesis of 3-[(2-ethylphenyl) amino]-2-{3-[(2S)-pyrrolidin-2-ylmethoxy]pyridin-4-yl}-1H,5H,6H,7H-pyrrolo[3,2-c] pyridin-4-one

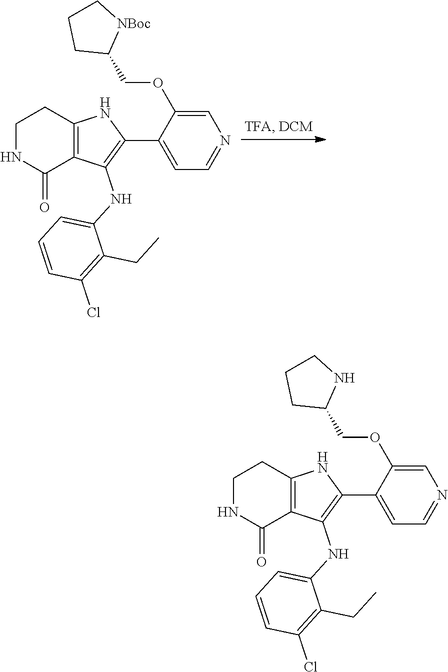 [SMILES: CCc1c(Cl)cccc1Nc1c(-c2ccncc2OC[C@@H]2CCCN2C(=O)OC(C)(C)C)[nH]c2c1C(=O)NCC2 ; CCc1c(Cl)cccc1Nc1c(-c2ccncc2OC[C@@H]2CCCN2)[nH]c2c1C(=O)NCC2]

To a stirred solution of tert-butyl (2S)-2-{[(4-{3-[(3-chloro-2-ethylphenyl) amino]-4-oxo-1H,5H,6H,7H-pyrrolo[3,2-c] pyridin-2-yl} pyridin-3-yl) oxy] methyl} pyrrolidine-1-carboxylate (90 mg, 0.159 mmol, 1 equiv) in DCM (0.9 mL) was added TFA (0.3 mL) at room temperature under nitrogen atmosphere. The resulting mixture was stirred for 1 h and concentrated under reduced pressure to afford 3-[(2-ethylphenyl) amino]-2-{3-[(2S)-pyrrolidin-2-ylmethoxy] pyridin-4-yl}-1H,5H,6H,7H-pyrrolo[3,2-c] pyridin-4-one (130 mg, crude) as a red oil.

LC-MS: M+H found: 466.0.

### 60.3. Synthesis of 3-[(3-chloro-2-ethylphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one

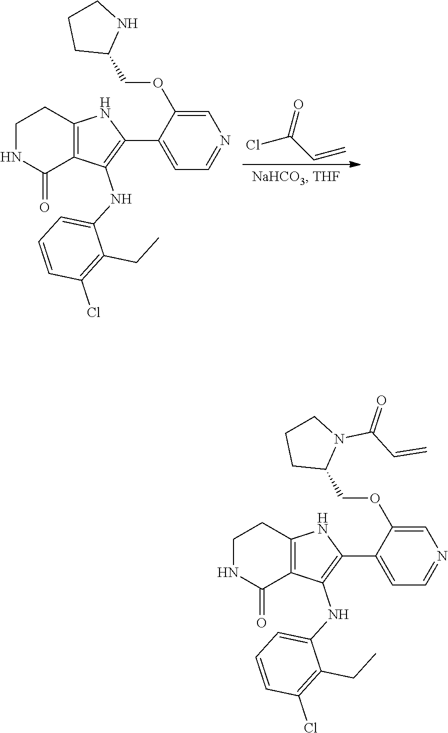

To a stirred solution of 3-[(2-ethylphenyl) amino]-2-{3-[(2S)-pyrrolidin-2-ylmethoxy]pyridin-4-yl}-1H,5H,6H,7H-pyrrolo[3,2-c] pyridin-4-one (130 mg, 0.181 mmol, 1 equiv) in THF (2 mL) were basified to pH 8 with NaHCO₃. To the above mixture was added acryloyl chloride (18.8 mg, 0.208 mmol, 1.15 equiv) at 0° C. under nitrogen atmosphere. The resulting mixture was stirred for 1 h at room temperature under nitrogen atmosphere. The mixture was extracted with CH₂Cl₂ (3×3 mL). The combined organic layers were washed with sat. NaCl (aq.) (10 mL), dried over anhydrous Na₂SO₄. After filtration, the filtrate was concentrated under reduced pressure. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 33% B to 50% B in 9 min, 50% B; Wave Length: 254/220 nm; RT1(min): 8.85; Number Of Runs: 0) to afford 3-[(3-chloro-2-ethylphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (5.9 mg, 6.18%) as a yellow solid.

LC-MS: M+H found: 520.00.

¹H NMR (300 MHz, Chloroform-d) δ 11.60 (s, 1H), 8.16 (s, 1H), 7.83 (d, 1H), 7.73 (s, 1H), 7.23 (s, 1H), 6.84 (d, 1H), 6.69 (t, 1H), 6.56-6.45 (m, 1H), 6.42 (d, 1H), 6.38-6.31 (m, 1H), 5.83-5.79 (m, 1H), 5.23 (s, 1H), 5.13-4.96 (m, 1H), 4.24 (t, 1H), 4.15-4.08 (m, 1H), 3.76 (t, 2H), 3.68-3.54 (m, 2H), 3.32-3.18 (m, 2H), 3.07-2.91 (m, 2H), 2.25-2.06 (m, 4H), 1.37 (t, 3H).

### Example 61. 3-[(2-ethyl-3-fluorophenyl) amino]-2-(3-{[(2S)-1-(prop-2-enoyl) pyrrolidin-2-yl] methoxy} pyridin-4-yl)-1H,5H,6H,7H-pyrrolo[3,2-c] pyridin-4-one (compound 211)

### 61.1. Synthesis of tert-butyl (2S)-2-{[(4-{3-[(2-ethyl-3-fluorophenyl) amino]-4-oxo-1H,5H,6H,7H-pyrrolo[3,2-c] pyridin-2-yl} pyridin-3-yl) oxy] methyl} pyrrolidine-1-carboxylate

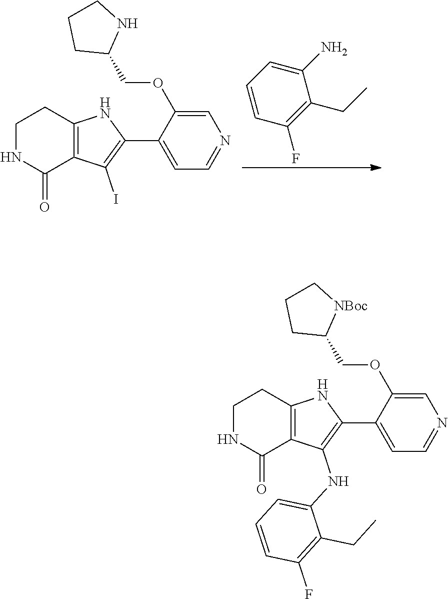

To a stirred solution of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (90 mg, 0.167 mmol, 1 equiv) and Cs₂CO₃ (109 mg, 0.334 mmol, 2 equiv) in DMF (1 mL) were added EPhos Pd G4 (15.4 mg, 0.017 mmol, 0.1 equiv) and 2-ethyl-3-fluoroaniline (27.9 mg, 0.200 mmol, 1.2 equiv) at room temperature under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (10:1) to afford tert-butyl (2S)-2-{[(4-{3-[(2-ethyl-3-fluorophenyl) amino]-4-oxo-1H,5H,6H,7H-pyrrolo[3,2-c] pyridin-2-yl}pyridin-3-yl) oxy] methyl} pyrrolidine-1-carboxylate (90 mg, 97.95%) as a yellow solid.

LC-MS: (M+H)⁺ found: 550.30.

### 61.2. Synthesis of 3-[(2-ethyl-3-fluorophenyl) amino]-2-{3-[(2S)-pyrrolidin-2-ylmethoxy] pyridin-4-yl}-1H,5H,6H,7H-pyrrolo[3,2-c] pyridin-4-one

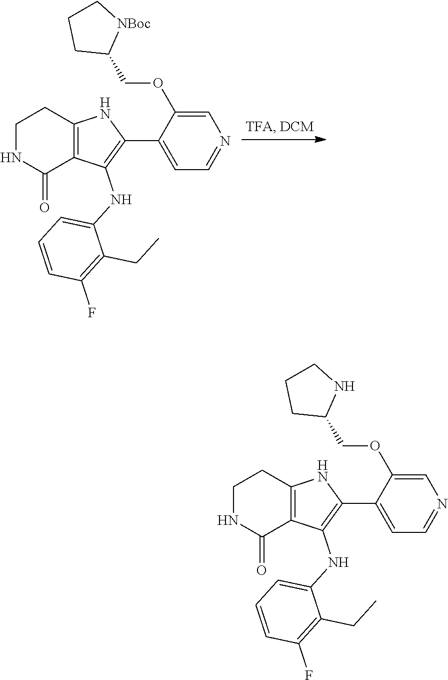 [SMILES: CCc1c(F)cccc1Nc1c(-c2ccncc2OC[C@@H]2CCCN2C(=O)OC(C)(C)C)[nH]c2c1C(=O)NCC2 ; CCc1c(F)cccc1Nc1c(-c2ccncc2OC[C@@H]2CCCN2)[nH]c2c1C(=O)NCC2]

To a stirred solution of tert-butyl (2S)-2-{[(4-{3-[(2-ethyl-3-fluorophenyl) amino]-4-oxo-1H,5H,6H,7H-pyrrolo [3,2-c] pyridin-2-yl} pyridin-3-yl) oxy] methyl} pyrrolidine-1-carboxylate (90 mg, 0.164 mmol, 1 equiv) in DCM (0.9 mL) was added TFA (0.3 mL) at room temperature under nitrogen atmosphere. The resulting mixture was stirred for 1 h and concentrated under reduced pressure to afford 3-[(2-ethyl-3-fluorophenyl) amino]-2-{3-[(2S)-pyrrolidin-2-ylmethoxy] pyridin-4-yl}-1H,5H,6H,7H-pyrrolo[3,2-c] pyridin-4-one (90 mg, crude) as a red oil.

LC-MS: (M+H)⁺ found: 450.20.

### 61.3. Synthesis of 3-[(2-ethyl-3-fluorophenyl) amino]-2-(3-{[(2S)-1-(prop-2-enoyl) pyrrolidin-2-yl] methoxy} pyridin-4-yl)-1H,5H,6H,7H-pyrrolo[3,2-c] pyridin-4-one

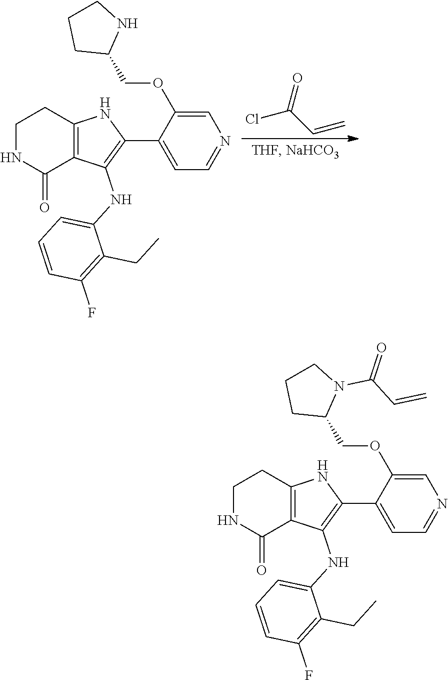

To a stirred solution of 3-[(2-ethyl-3-fluorophenyl) amino]-2-{3-[(2S)-pyrrolidin-2-ylmethoxy] pyridin-4-yl}-1H,5H,6H,7H-pyrrolo[3,2-c] pyridin-4-one (90 mg, 0.160 mmol, 1 equiv) in THF (0.5 mL) basified to pH 8 with NaHCO₃(aq.). To the above mixture was added acryloyl chloride (16.6 mg, 0.184 mmol, 1.15 equiv) at 0° C. under nitrogen atmosphere. The resulting mixture was stirred for 1 h at room temperature under nitrogen atmosphere. The mixture was extracted with CH₂Cl₂ (3×3 mL). The combined organic layers were washed with sat. NaCl (aq.) (10 mL), dried over anhydrous Na₂SO₄. After filtration, the filtrate was concentrated under reduced pressure. The crude product (100 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 30% B to 60% B in 7 min; Wave Length: 254 nm; RT1(min): 6.5; Number Of Runs: 0) to afford 3-[(2-ethyl-3-fluorophenyl) amino]-2-(3-{[(2S)-1-(prop-2-enoyl) pyrrolidin-2-yl] methoxy} pyridin-4-yl)-1H,5H,6H,7H-pyrrolo[3,2-c] pyridin-4-one (11.6 mg, 13.96%) as a yellow solid.

LC-MS: (M+H)⁺ found: 504.15.

¹H NMR (300 MHz, Chloroform-d) δ 11.53 (s, 1H), 8.17 (s, 1H), 7.86 (d, 1H), 7.60 (s, 1H), 7.29 (d, 1H), 6.76-6.33 (m, 4H), 6.21 (d, 1H), 5.85-5.79 (m, 1H), 5.22 (s, 1H), 5.14-4.98 (m, 1H), 4.24 (t, 1H), 4.16-4.10 (m, 1H), 3.76 (t, 2H), 3.67-3.50 (m, 2H), 3.23 (t, 2H), 2.95-2.78 (m, 2H), 2.25-2.05 (m, 4H), 1.45-1.31 (m, 3H).

### Example 62. 2-(3-{[(2R)-1-(but-2-ynoyl) azetidin-2-yl] methoxy} pyridin-4-yl)-3-[(3-chloro-2-methoxyphenyl) amino]-1H,5H,6H,7H-pyrrolo[3,2-c] pyridin-4-one (compound 227)

### 62.1. Synthesis of 2-{3-[(2R)-azetidin-2-ylmethoxy]pyridin-4-yl}-3-[(3-chloro-2-methoxyphenyl)amino]-1H,5H,6H,7H-pyrrolo[3,2-c]pyridin-4-one

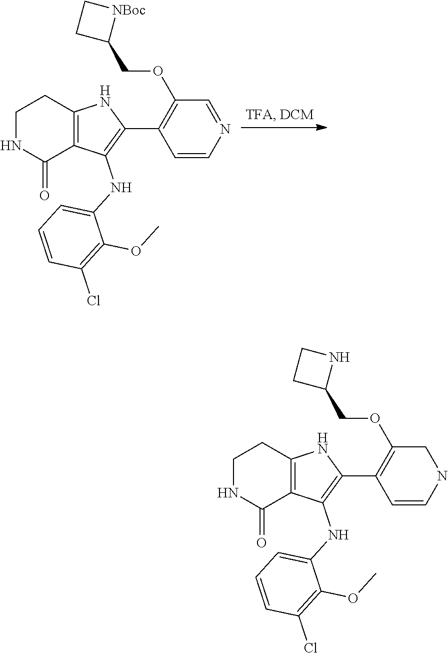 [SMILES: COc1c(Cl)cccc1Nc1c(-c2ccncc2OC[C@H]2CCN2C(=O)OC(C)(C)C)[nH]c2c1C(=O)NCC2 ; COc1c(Cl)cccc1Nc1c(C2=C(OC[C@H]3CCN3)CNC=C2)[nH]c2c1C(=O)NCC2]

To a stirred solution of tert-butyl (2S)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]azetidine-1-carboxylate (670 mg, 1.02 mmol, 1 equiv) in DCM (6 mL) was added TFA (2 mL) at room temperature under nitrogen atmosphere. The resulting mixture was stirred for 1 h and concentrated under reduced pressure to afford 2-{3-[(2R)-azetidin-2-ylmethoxy]pyridin-4-yl}-3-[(3-chloro-2-methoxyphenyl)amino]-1H,5H,6H,7H-pyrrolo[3,2-c]pyridin-4-one (1.2 g, crude) as a red oil.

LC-MS: M+H found: 454.1.

### 62.2. Synthesis of 2-(3-{[(2R)-1-(but-2-ynoyl) azetidin-2-yl] methoxy} pyridin-4-yl)-3-[(3-chloro-2-methoxyphenyl) amino]-1H,5H,6H,7H-pyrrolo[3,2-c] pyridin-4-one

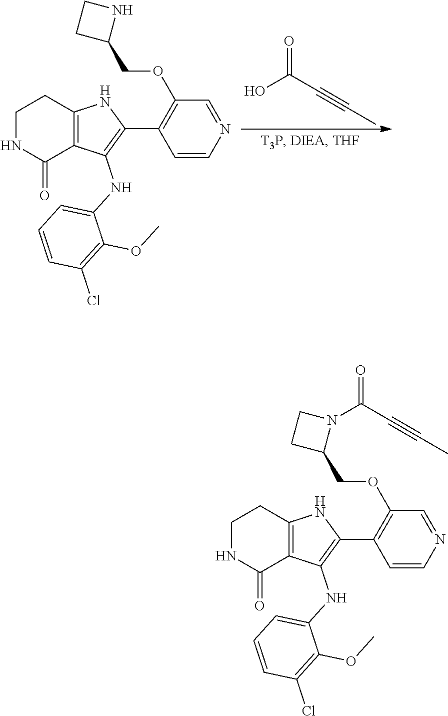

A mixture of 2-{3-[(2R)-azetidin-2-ylmethoxy] pyridin-4-yl}-3-[(3-chloro-2-methoxyphenyl) amino]-1H,5H,6H,7H-pyrrolo[3,2-c] pyridin-4-one (230 mg, 0.220 mmol, 1 equiv) in THF (1 mL) was basified to pH 8 with DIEA. To the above mixture was added 2-butynoic acid (27.7 mg, 0.330 mmol, 1.5 equiv) at 0° C. under nitrogen atmosphere followed by the addition of T3P (209 mg, 0.330 mmol, 1.5 equiv, 50% in EA) dropwise. The resulting mixture was stirred for 1 h at room temperature under nitrogen atmosphere. Saturated aqueous NaHCO₃ (10 mL) was added to the reaction mixture at 0° C. and extracted with EtOAc (3×10 mL), dried over anhydrous Na₂SO₄ and filtered. The filtrate was concentrated under reduced pressure to give crude product (100 mg) that was purified by Prep-HPLC under the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 27% B to 57% B in 7 min, 57% B; Wave Length: 254 nm; RT1(min): 5.57; Number Of Runs: 0) to afford 2-(3-{[(2R)-1-(but-2-ynoyl) azetidin-2-yl] methoxy} pyridin-4-yl)-3-[(3-chloro-2-methoxyphenyl) amino]-1H,5H,6H,7H-pyrrolo[3,2-c] pyridin-4-one (16.3 mg, 14.0%) as a yellow solid.

LC-MS: M+H found: 519.95.

¹H NMR (400 MHz, Chloroform-d) δ 11.40 (s, 1H), 8.25 (s, 1H), 7.98 (d, 1H), 7.53 (s, 1H), 7.44 (d, 1H), 6.80-6.70 (m, 1H), 6.60 (t, 1H), 6.20-6.18 (m, 1H), 5.19 (s, 1H), 5.01-4.87 (m, 1H), 4.49 (t, 1H), 4.31-4.19 (m, 3H), 4.07 (s, 3H), 3.64-3.51 (m, 2H), 3.11-3.00 (m, 2H), 2.72-2.49 (m, 1H), 2.20-2.10 (m, 1H), 2.03 (s, 3H).

### Example 63. 3-[(3-chloro-2-methoxyphenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one (compound 226)

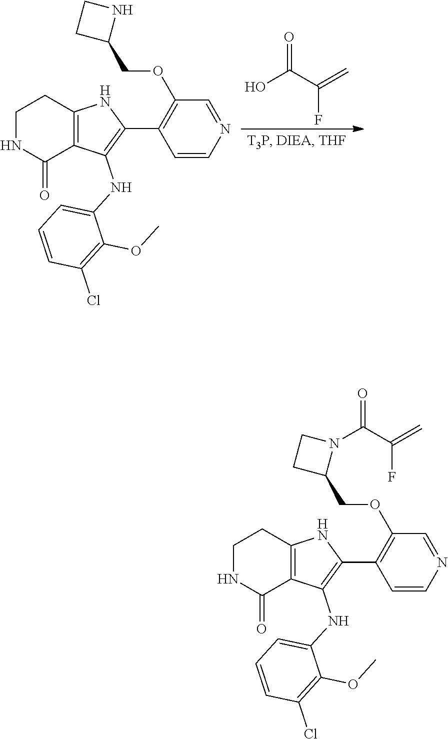

To a stirred solution of 2-{3-[(2R)-azetidin-2-ylmethoxy] pyridin-4-yl}-3-[(3-chloro-2-methoxyphenyl) amino]-1H,5H,6H,7H-pyrrolo[3,2-c] pyridin-4-one (230 mg, 0.220 mmol, 1 equiv) in THF (1 mL) was basified to pH 8 with DIEA. To the above mixture was added 2-fluoroprop-2-enoic acid (29.7 mg, 0.330 mmol, 1.5 equiv) at 0° C. under nitrogen atmosphere followed by the addition of T3P (210 mg, 0.330 mmol, 1.5 equiv, 50% in EA) dropwise. The resulting mixture was stirred for 1 h at room temperature under nitrogen atmosphere. Saturated aqueous NaHCO₃ (10 mL) was added to the reaction mixture at 0° C. and extracted with EtOAc (3×10 mL), dried over anhydrous Na₂SO₄ and filtered. The filtrate was concentrated under reduced pressure to give crude product (100 mg) that was purified by Prep-HPLC under the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 28% B to 52% B in 9 min, 52% B; Wave Length: 254/220 nm; RT1(min): 7.53; Number Of Runs: 0) to afford 3-[(3-chloro-2-methoxyphenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one (25.2 mg, 21.55%) as a yellow solid.

LC-MS: M+H found: 525.90.

¹H NMR (300 MHz, DMSO-d6) δ 11.53 (s, 1H), 8.40 (s, 1H), 8.01 (d, 1H), 7.49 (s, 1H), 7.35 (d, 1H), 7.12 (s, 1H), 6.75-6.57 (m, 2H), 6.19-6.04 (m, 1H), 5.65-5.26 (m, 2H), 4.98-4.72 (m, 1H), 4.64-4.40 (m, 2H), 4.32 (s, 2H), 3.89 (s, 3H), 3.45-3.39 (m, 2H), 2.99-2.81 (m, 2H), 2.62-2.52 (m, 1H), 2.26-2.02 (m, 1H).

### Example 64. 3-[(3-chloro-2-methoxyphenyl)amino]-2-(3-{2-[(2S,5S)-5-methyl-1-(prop-2-enoyl)pyrrolidin-2-yl]ethynyl}pyridin-4-yl)-1H,5H,6H,7H-pyrrolo[3,2-c]pyridin-4-one (compound 240)

### 64.1. Synthesis of tert-butyl (2S,5S)-2-(hydroxymethyl)-5-methylpyrrolidine-1-carboxylate

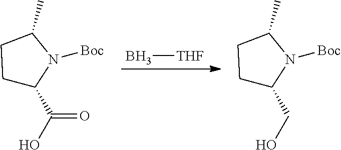

To a stirred solution of (2S,5S)-1-(tert-butoxycarbonyl)-5-methylpyrrolidine-2-carboxylic acid (3.00 g, 13.1 mmol, 1 equiv) in THF (30 mL) was added BH₃-THF (1.35 g, 15.7 mmol, 1.20 equiv) at 0 degrees C. under N₂ atmosphere. The resulting mixture was stirred for 1 h at 25 degrees C. The resulting solution was quenched by the addition of MeOH (100 mL). The mixture was concentrated under vacuum to afford tert-butyl (2S,5S)-2-(hydroxymethyl)-5-methylpyrrolidine-1-carboxylate (3 g, crude) as a white oil. 64.2. Synthesis of tert-butyl (2S,5S)-2-formyl-5-methylpyrrolidine-1-carboxylate

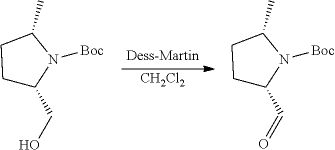

To a stirred solution of tert-butyl (2S,5S)-2-(hydroxymethyl)-5-methylpyrrolidine-1-carboxylate (3 g, 13.9 mmol, 1 equiv) in DCM (30 mL) was added Dess-Martin (8.88 g, 20.9 mmol, 1.5 equiv) at 0 degrees C. under N₂ atmosphere. The resulting mixture was stirred for 1 h at 25 degrees C. The resulting solution was quenched by the addition of Na₂SO₃ (5 mL). The mixture was neutralized to pH 7 with Na₂CO₃. The mixture was extracted with DCM (3×20 mL). The combined organic layers were washed with NaCl (3×50 mL), dried over anhydrous Na₂SO₄ and concentrated under vacuum. The residue was purified by silica gel column chromatography, eluted with PE/EA (20/1) to afford tert-butyl (2S,5S)-2-formyl-5-methylpyrrolidine-1-carboxylate (2.33 g, 80.5%) as a white oil.

### 64.3. Synthesis of tert-butyl (2S,5S)-2-ethynyl-5-methylpyrrolidine-1-carboxylate

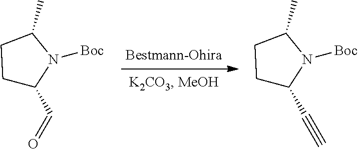

To a stirred solution of tert-butyl (2S,5S)-2-formyl-5-methylpyrrolidine-1-carboxylate (2 g, 9.37 mmol, 1.00 equiv) and K₂CO₃ (2.59 g, 18.7 mmol, 2.00 equiv) in MeOH (30 mL) were added Bestmann-Ohira reagent (2.16 g, 11.2 mmol, 1.20 equiv) at 0 degrees C. under N₂ atmosphere. The resulting mixture was stirred for 2 h at 25 degrees C. The resulting solution was quenched by sat. potassium sodium tartrate (aq.) (5 mL) at 0° C. The mixture was extracted with EA (3×20 mL). The combined organic layers were washed with NaCl (aq.) (3×50 mL), dried over anhydrous Na₂SO₄ and concentrated under vacuum. The residue was purified by silica gel column chromatography, eluted with PE/EA (20/1) to afford tert-butyl (2S,5S)-2-ethynyl-5-methylpyrrolidine-1-carboxylate (1.5 g, 68.79%) as a white oil.

### 64.4. Synthesis of tert-butyl (2S,5S)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate

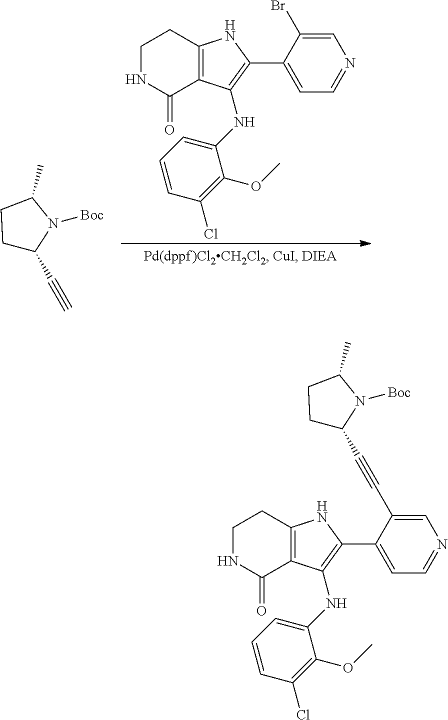 [SMILES: C#C[C@@H]1CC[C@H](C)N1C(=O)OC(C)(C)C ; COc1c(Cl)cccc1Nc1c(-c2ccncc2Br)[nH]c2c1C(=O)NCC2 ; COc1c(Cl)cccc1Nc1c(-c2ccncc2C#C[C@@H]2CC[C@H](C)N2C(=O)OC(C)(C)C)[nH]c2c1C(=O)NCC2]

To a stirred solution of 3-[(3-chloro-2-methoxyphenyl)amino]-2-(3-iodopyridin-4-yl)-1H,5H,6H,7H-pyrrolo[3,2-c]pyridin-4-one (200 mg, 0.404 mmol, 1.00 equiv), tert-butyl (2S,5S)-2-ethynyl-5-methylpyrrolidine-1-carboxylate (254 mg, 1.21 mmol, 3 equiv), CuI (38.5 mg, 0.202 mmol, 0.5 equiv), DIEA (261 mg, 2.02 mmol, 5 equiv) and Pd(dppf)Cl2CH2Cl2 (82.3 mg, 0.101 mmol, 0.25 equiv) in DMF (5 mL) at room temperature under argon atmosphere. The resulting mixture was stirred for overnight at 50° C. under argon atmosphere. The resulting mixture was concentrated under vacuum. The residue was purified by silica gel column chromatography, eluted with CH2Cl2/MeOH (25:1) to afford tert-butyl (2S,5S)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate (120 mg, 51.53%) as a yellow solid.

LC-MS: (M+H)⁺ found 576.25

### 64.5. Synthesis of 3-[(3-chloro-2-methoxyphenyl)amino]-2-(3-{2-[(2S,5S)-5-methylpyrrolidin-2-yl]ethynyl}pyridin-4-yl)-1H,5H,6H,7H-pyrrolo[3,2-c]pyridin-4-one

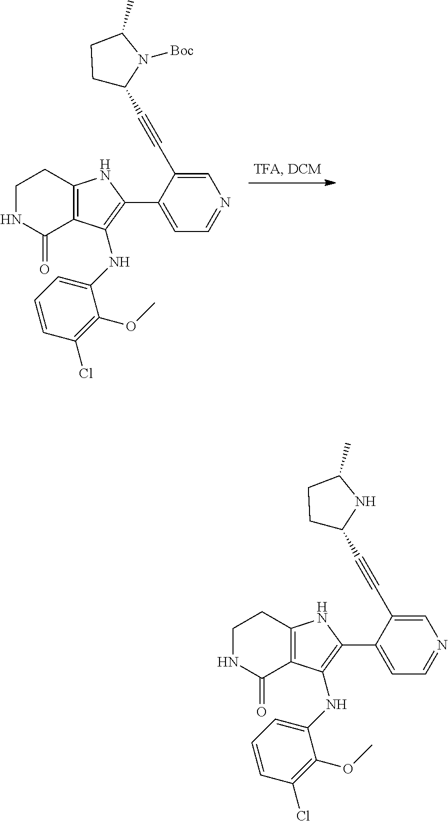 [SMILES: COc1c(Cl)cccc1Nc1c(-c2ccncc2C#C[C@@H]2CC[C@H](C)N2C(=O)OC(C)(C)C)[nH]c2c1C(=O)NCC2 ; COc1c(Cl)cccc1Nc1c(-c2ccncc2C#C[C@@H]2CC[C@H](C)N2)[nH]c2c1C(=O)NCC2]

To a stirred solution of tert-butyl (2S,5S)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate (100 mg, 0.174 mmol, 1 equiv) in DCM (3 mL) was added TFA (1 mL) at room temperature under nitrogen atmosphere. The resulting mixture was stirred for 1 h and concentrated under reduced pressure to afford 3-[(3-chloro-2-methoxyphenyl)amino]-2-(3-{2-[(2S,5S)-5-methylpyrrolidin-2-yl]ethynyl}pyridin-4-yl)-1H,5H,6H,7H-pyrrolo[3,2-c]pyridin-4-one (120 mg, crude) as a yellow oil.

LC-MS: (M+H)⁺ found 476.20

### 64.6. Synthesis of 3-[(3-chloro-2-methoxyphenyl)amino]-2-(3-{2-[(2S,5S)-5-methyl-1-(prop-2-enoyl)pyrrolidin-2-yl]ethynyl}pyridin-4-yl)-1H,5H,6H,7H-pyrrolo[3,2-c]pyridin-4-one

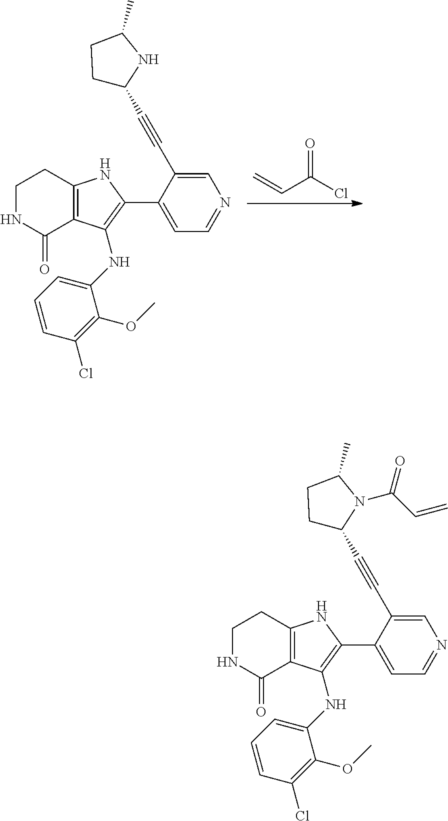

To a stirred solution of 3-[(3-chloro-2-methoxyphenyl)amino]-2-(3-{2-[(2S,5S)-5-methylpyrrolidin-2-yl]ethynyl}pyridin-4-yl)-1H,5H,6H,7H-pyrrolo[3,2-c]pyridin-4-one (100 mg, 0.210 mmol, 1 equiv) in THF (3 mL) was basified to pH 8 with saturated sat. NaHCO₃(aq.). To the above mixture was added acryloyl chloride (38 mg, 0.420 mmol, 2 equiv) dropwise at 0° C. The resulting mixture was stirred for 30 min at room temperature. The reaction was quenched with MeOH (0.5 mL) at 0° C. The mixture was extracted with CH2Cl2/MeOH (10/1) (2×10 mL), dried over anhydrous Na₂SO₄ and filtered. The filtrate was concentrated under reduced pressure to give crude product (100 mg) that was purified by Prep-HPLC under the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 34% B to 53% B in 8 min, 53% B; Wave Length: 254/220 nm; RT1(min): 8) to afford 3-[(3-chloro-2-methoxyphenyl)amino]-2-(3-{2-[(2S,5S)-5-methyl-1-(prop-2-enoyl)pyrrolidin-2-yl]ethynyl}pyridin-4-yl)-1H,5H,6H,7H-pyrrolo[3,2-c]pyridin-4-one (34.9 mg, 31.18%) as a yellow solid.

LC-MS: (M+H)⁺ found 530.10.

¹H NMR (400 MHz, Chloroform-d) δ 11.25 (s, 1H), 8.55 (s, 1H), 8.12 (d, 1H), 7.80 (s, 1H), 7.42 (d, 1H), 6.73-6.71 (m, 1H), 6.62-6.57 (m, 1H), 6.54-6.50 (m, 1H), 6.45-6.40 (m, 1H), 6.23-6.21 (m, 1H), 5.79-5.76 (m, 1H), 5.25 (s, 1H), 4.89-4.80 (m, 1H), 4.38-4.25 (m, 1H), 4.07 (s, 3H), 3.68-3.52 (m, 2H), 3.32-3.21 (m, 2H), 2.49-2.30 (m, 2H), 2.22-2.10 (m, 1H), 1.98-1.90 (m, 1H), 1.42 (d, 3H).

### Example 65. 3-[(3-chloro-2-methoxyphenyl)amino]-2-(3-{2-[(2S,5R)-5-methyl-1-(prop-2-enoyl)pyrrolidin-2-yl]ethynyl}pyridin-4-yl)-1H,5H,6H,7H-pyrrolo[3,2-c]pyridin-4-one (compound 239)

### 65.1. Synthesis of tert-butyl (2S,5R)-2-(hydroxymethyl)-5-methylpyrrolidine-1-carboxylate

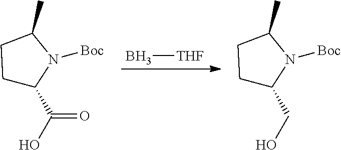

To a stirred solution of (2S,5R)-1-(tert-butoxycarbonyl)-5-methylpyrrolidine-2-carboxylic acid (900 mg, 3.925 mmol, 1 equiv) in THF (9 mL) was added BH₃-THF (506 mg, 5.89 mmol, 1.5 equiv) at 0° C. under nitrogen atmosphere. The resulting mixture was stirred for 1 h at room temperature under nitrogen atmosphere. The resulting solution was quenched by the addition of MeOH (20 mL). The mixture was concentrated under vacuum to afford tert-butyl (2S,5R)-2-(hydroxymethyl)-5-methylpyrrolidine-1-carboxylate (850 mg, crude) as a yellow oil.

LC-MS: M+H found: 216.0.

### 65.2. Synthesis of tert-butyl (2S,5R)-2-formyl-5-methylpyrrolidine-1-carboxylate

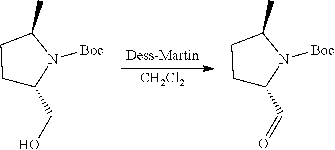

To a stirred solution of tert-butyl (2S,5R)-2-(hydroxymethyl)-5-methylpyrrolidine-1-carboxylate (870 mg, 4.04 mmol, 1 equiv) in methylene chloride (9 mL) was added Dess-Martin (2.06 g, 4.85 mmol, 1.2 equiv) in portions at 0° C. under nitrogen atmosphere. The resulting mixture was stirred for 1 h at room temperature under nitrogen atmosphere. The reaction was quenched with sat. NaHCO₃(aq.) at 0° C. The mixture was extracted with CH₂Cl₂ (3×10 mL). The combined organic layers were washed with sat. NaCl (aq.) (20 mL), dried over anhydrous Na₂SO₄ and concentrated under vacuum. The residue was purified by silica gel column chromatography, eluted with PE/EA (30:1) to afford tert-butyl (2S,5R)-2-formyl-5-methylpyrrolidine-1-carboxylate (700 mg, 81.22%) as a white oil.

LC-MS: M+H found: 214.0.

### 65.3. Synthesis of tert-butyl (2S,5R)-2-ethynyl-5-methylpyrrolidine-1-carboxylate

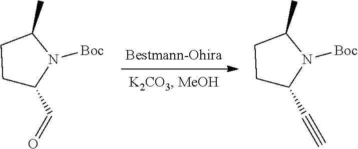

To a stirred solution of tert-butyl (2S,5R)-2-formyl-5-methylpyrrolidine-1-carboxylate (650 mg, 3.05 mmol, 1 equiv) and K₂CO₃ (842 mg, 6.10 mmol, 2 equiv) in MeOH (10 mL) were added dimethyl (1-diazo-2-oxopropyl) phosphonate (878 mg, 4.57 mmol, 1.5 equiv) in portions at 0° C. under nitrogen atmosphere. The resulting mixture was stirred for 1 h at room temperature under nitrogen atmosphere. The resulting solution was quenched by sat. potassium sodium tartrate (aq.) (10 mL) at 0° C. The mixture was extracted with EA (3×20 mL). The combined organic layers were washed with sat. NaCl (aq.) (20 mL), dried over anhydrous Na₂SO₄ and concentrated under vacuum. The residue was purified by silica gel column chromatography, eluted with PE/EA (30:1) to afford tert-butyl (2S,5R)-2-ethynyl-5-methylpyrrolidine-1-carboxylate (450 mg, 70.55%) as a white oil.

LC-MS: M+H found: 210.0.

### 65.4. Synthesis of -[(3-chloro-2-methoxyphenyl) amino]-2-(3-{2-[(2S,5R)-5-methyl-1-(prop-2-enoyl) pyrrolidin-2-yl] ethynyl} pyridin-4-yl)-1H,5H,6H,7H-pyrrolo[3,2-c]pyridin-4-one

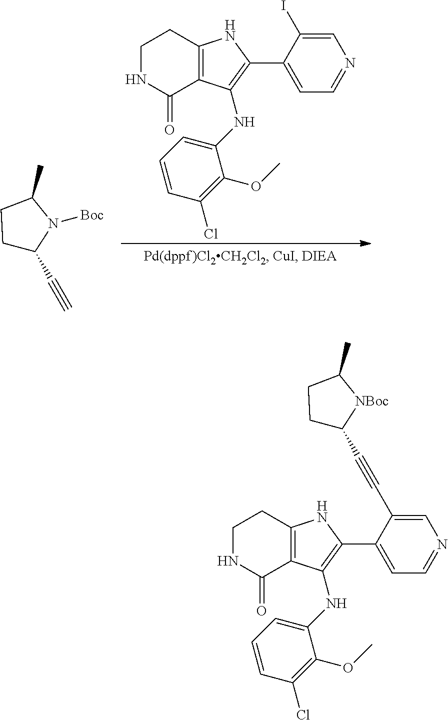 [SMILES: C#C[C@@H]1CC[C@@H](C)N1C(=O)OC(C)(C)C ; COc1c(Cl)cccc1Nc1c(-c2ccncc2I)[nH]c2c1C(=O)NCC2 ; COc1c(Cl)cccc1Nc1c(-c2ccncc2C#C[C@@H]2CC[C@@H](C)N2C(=O)OC(C)(C)C)[nH]c2c1C(=O)NCC2]

To a stirred solution of 3-[(3-chloro-2-methoxyphenyl)amino]-2-(3-iodopyridin-4-yl)-1H,5H,6H,7H-pyrrolo[3,2-c]pyridin-4-one (200 mg, 0.404 mmol, 1.00 equiv) and CuI (38.5 mg, 0.202 mmol, 0.5 equiv) in DMF (3 mL) were added Pd(dppf)Cl₂·CH₂Cl₂ (164 mg, 0.202 mmol, 0.5 equiv) and DIEA (157 mg, 1.21 mmol, 3 equiv) and tert-butyl (2S,5R)-2-ethynyl-5-methylpyrrolidine-1-carboxylate (211 mg, 1.01 mmol, 2.50 equiv) at room temperature under argon atmosphere. The resulting mixture was stirred for 2 h at 50° C. under argon atmosphere. The resulting mixture was concentrated under vacuum. The residue was purified by reverse flash chromatography with the following conditions: column, silica gel; mobile phase, MeCN in water, 10% to 50% gradient in 10 min; detector, UV 254 nm to afford 3-[(3-chloro-2-methoxyphenyl) amino]-2-(3-{2-[(2S,5R)-5-methyl-1-(prop-2-enoyl) pyrrolidin-2-yl] ethynyl} pyridin-4-yl)-1H,5H,6H,7H-pyrrolo[3,2-c] pyridin-4-one (150 mg, 70.00%) as a yellow solid.

LC-MS: M+H found: 576.0.

### 65.5. Synthesis of 3-[(3-chloro-2-methoxyphenyl) amino]-2-(3-{2-[(2S,5R)-5-methylpyrrolidin-2-yl] ethynyl} pyridin-4-yl)-1H,5H,6H,7H-pyrrolo[3,2-c] pyridin-4-one

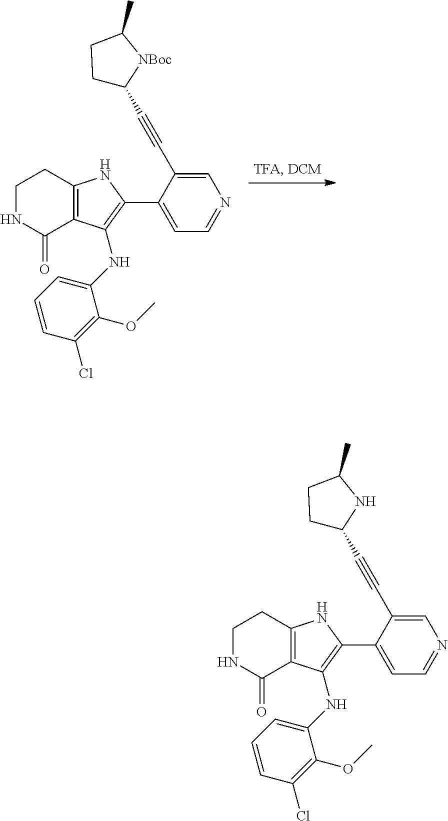 [SMILES: COc1c(Cl)cccc1Nc1c(-c2ccncc2C#C[C@@H]2CC[C@@H](C)N2C(=O)OC(C)(C)C)[nH]c2c1C(=O)NCC2 ; COc1c(Cl)cccc1Nc1c(-c2ccncc2C#C[C@@H]2CC[C@@H](C)N2)[nH]c2c1C(=O)NCC2]

To a stirred solution of 3-[(3-chloro-2-methoxyphenyl) amino]-2-(3-{2-[(2S,5R)-5-methyl-1-(prop-2-enoyl) pyrrolidin-2-yl] ethynyl} pyridin-4-yl)-1H,5H,6H,7H-pyrrolo[3,2-c] pyridin-4-one (140 mg, 0.264 mmol, 1 equiv) in DCM (1.5 mL) was added TFA (0.5 mL) at room temperature under nitrogen atmosphere. The resulting mixture was stirred for 1 h and concentrated under reduced pressure to afford 3-[(3-chloro-2-methoxyphenyl) amino]-2-(3-{2-[(2S,5R)-5-methylpyrrolidin-2-yl] ethynyl} pyridin-4-yl)-1H,5H,6H,7H-pyrrolo[3,2-c] pyridin-4-one (180 mg, crude) as a red oil.

LC-MS: M+H found: 476.0.

### 65.6. Synthesis of 3-[(3-chloro-2-methoxyphenyl)amino]-2-(3-{2-[(2S,5R)-5-methyl-1-(prop-2-enoyl)pyrrolidin-2-yl]ethynyl}pyridin-4-yl)-1H,5H,6H,7H-pyrrolo[3,2-c]pyridin-4-one

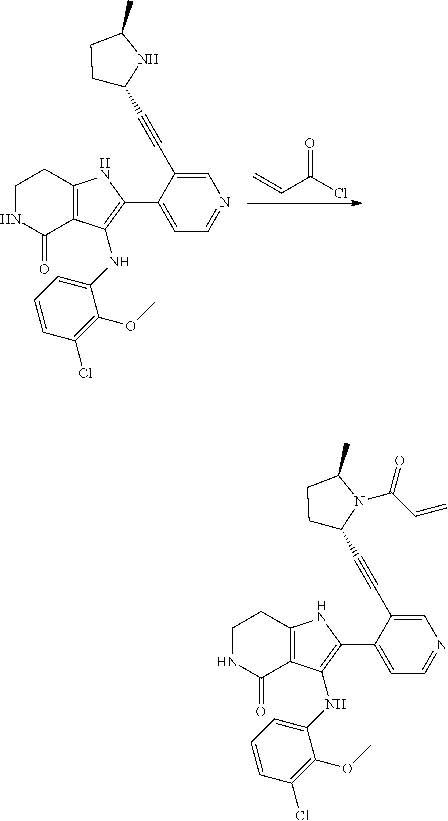

To a stirred solution of 3-[(3-chloro-2-methoxyphenyl) amino]-2-(3-{2-[(2S,5R)-5-methylpyrrolidin-2-yl] ethynyl} pyridin-4-yl)-1H,5H,6H,7H-pyrrolo[3,2-c] pyridin-4-one (180 mg, 0.265 mmol, 1 equiv) in THF (2 mL) was basified to pH 8 with sat. NaHCO₃(aq.). To the above mixture was added acryloyl chloride (27.5 mg, 0.305 mmol, 1.15 equiv) at 0° C. under nitrogen atmosphere. The resulting mixture was stirred for 1 h at room temperature under nitrogen atmosphere. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (130 mg) that was purified by Prep-HPLC under the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 38% B to 54% B in 8 min, 54% B; Wave Length: 254/220 nm; RT1(min): 8; Number Of Runs: 0) to afford 3-[(3-chloro-2-methoxyphenyl)amino]-2-(3-{2-[(2S,5R)-5-methyl-1-(prop-2-enoyl)pyrrolidin-2-yl]ethynyl}pyridin-4-yl)-1H,5H,6H,7H-pyrrolo[3,2-c]pyridin-4-one (14.2 mg, 10.05%) as a yellow solid.

LC-MS: M+H found: 530.10.

¹H NMR (300 MHz, Chloroform-d) δ 11.06 (s, 1H), 8.53 (s, 1H), 8.15 (s, 1H), 7.72 (s, 1H), 7.44 (s, 1H), 6.78-6.71 (m, 1H), 6.65-6.38 (m, 3H), 6.27-6.12 (m, 1H), 5.83-5.67 (m, 1H), 5.26 (s, 1H), 5.01 (d, 1H), 4.46-4.28 (m, 1H), 4.07 (s, 3H), 3.77-3.49 (m, 2H), 3.38-3.14 (m, 2H), 2.58-2.22 (m, 3H), 1.95-1.86 (m, 1H), 1.30 (d, 3H).

### Example 66. 3-[(3-chloro-2-methoxyphenyl)amino]-2-(3-{2-[(2R,5R)-5-methyl-1-(prop-2-enoyl)pyrrolidin-2-yl]ethynyl}pyridin-4-yl)-1H,5H,6H,7H-pyrrolo[3,2-c]pyridin-4-one (compound 238)

### 66.1. Synthesis of tert-butyl (2R,5R)-2-(hydroxymethyl)-5-methylpyrrolidine-1-carboxylate

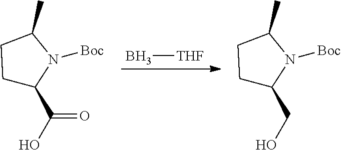

To a stirred solution of (2R,5R)-1-(tert-butoxycarbonyl)-5-methylpyrrolidine-2-carboxylic acid (1 g, 4.36 mmol, 1 equiv) in THF (10 mL) was added BH₃-THF (6.6 mL, 1.5 equiv) dropwise at 0° C. under nitrogen atmosphere. The resulting mixture was stirred for 1 h at 75° C. under nitrogen atmosphere. The reaction was quenched with MeOH (5 mL) at 0° C. The mixture was concentrated under vacuum to afford tert-butyl (2R,5R)-2-(hydroxymethyl)-5-methylpyrrolidine-1-carboxylate (900 mg, crude) as a colourless oil.

### 66.2. Synthesis of tert-butyl (2R,5R)-2-formyl-5-methylpyrrolidine-1-carboxylate

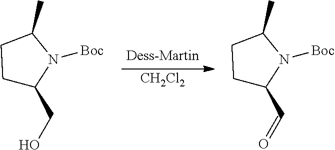

To a stirred solution of tert-butyl (2R,5R)-2-(hydroxymethyl)-5-methylpyrrolidine-1-carboxylate (900 mg, 4.18 mmol, 1 equiv) in DCM (20 mL) was added Dess-Martin (1.95 g, 4.60 mmol, 1.1 equiv) in portions at 0° C. under nitrogen atmosphere. The resulting mixture was stirred for 1 h at room temperature under nitrogen atmosphere. The reaction was quenched with sat. sodium sulfite (aq.) (5 mL) at 0° C. The mixture was extracted with CH₂Cl₂ (2×20 mL). The organic layer was concentrated under vacuum. The residue was purified by silica gel column chromatography, eluted with PE/EA (30:1) to afford tert-butyl (2R,5R)-2-formyl-5-methylpyrrolidine-1-carboxylate (790 mg, 88.6%) as a colorless oil.

### 66.3. Synthesis of tert-butyl (2R,5R)-2-ethynyl-5-methylpyrrolidine-1-carboxylate

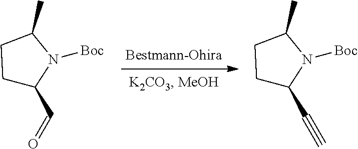

To a stirred solution of tert-butyl (2R,5R)-2-formyl-5-methylpyrrolidine-1-carboxylate (790 mg, 3.70 mmol, 1 equiv) and K₂CO₃ (1.02 g, 7.41 mmol, 2 equiv) in MeOH (30 mL) was added Bestmann-Ohira reagent (854 mg, 4.45 mmol, 1.2 equiv) dropwise at 0° C. under nitrogen atmosphere. The resulting mixture was stirred for 1 h at room temperature under nitrogen atmosphere. The reaction was quenched with sat. potassium sodium tartrate (aq.) (5 mL) at 0° C. The mixture was extracted with EtOAc (2×30 mL). The organic layer was concentrated under vacuum. The residue was purified by silica gel column chromatography, eluted with PE/EA (20:1) to afford tert-butyl (2R,5R)-2-ethynyl-5-methylpyrrolidine-1-carboxylate (610 mg, 78.7%) as a colorless oil.

### 66.4. Synthesis of tert-butyl (2R,5R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate

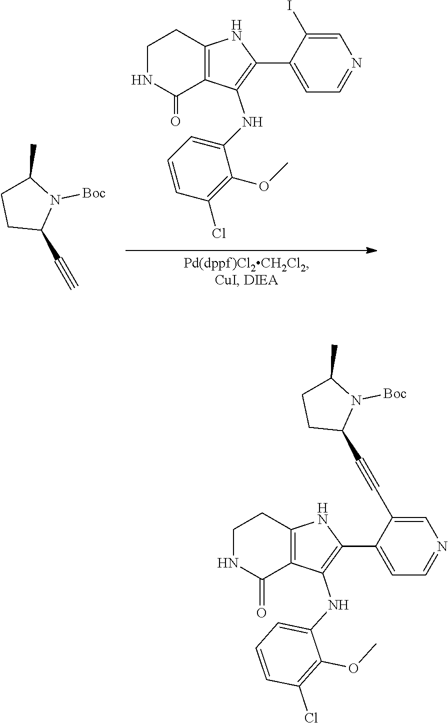 [SMILES: C#C[C@H]1CC[C@@H](C)N1C(=O)OC(C)(C)C ; COc1c(Cl)cccc1Nc1c(-c2ccncc2I)[nH]c2c1C(=O)NCC2 ; COc1c(Cl)cccc1Nc1c(-c2ccncc2C#C[C@H]2CC[C@@H](C)N2C(=O)OC(C)(C)C)[nH]c2c1C(=O)NCC2]

A mixture of 3-[(3-chloro-2-methoxyphenyl)amino]-2-(3-iodopyridin-4-yl)-1H,5H,6H,7H-pyrrolo[3,2-c]pyridin-4-one (100 mg, 0.202 mmol, 1.00 equiv), CuI (19.3 mg, 0.101 mmol, 0.5 equiv), DIEA (131 mg, 1.01 mmol, 5 equiv), Pd(dppf)Cl2CH2Cl2 (91 mg, 0.11 mmol, 0.25 equiv) and tert-butyl (2R,5R)-2-ethynyl-5-methylpyrrolidine-1-carboxylate (84.6 mg, 0.404 mmol, 2 equiv) in DMF (3 mL) at room temperature under argon atmosphere. The resulting mixture was stirred for 4 h at 50° C. under argon atmosphere. The mixture was concentrated under vacuum. The residue was purified by reverse flash chromatography with the following conditions: column, C18 silica gel; mobile phase, MeCN in water, 10% to 80% gradient in 10 min; detector, UV 254 nm. This resulted in tert-butyl (2R,5R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate (100 mg, 85.8%) as a yellow oil.

LC-MS: (M+H)⁺ found 576.10

### 66.5. Synthesis of 3-[(3-chloro-2-methoxyphenyl)amino]-2-(3-{2-[(2R,5R)-5-methylpyrrolidin-2-yl]ethynyl}pyridin-4-yl)-1H,5H,6H,7H-pyrrolo[3,2-c]pyridin-4-one

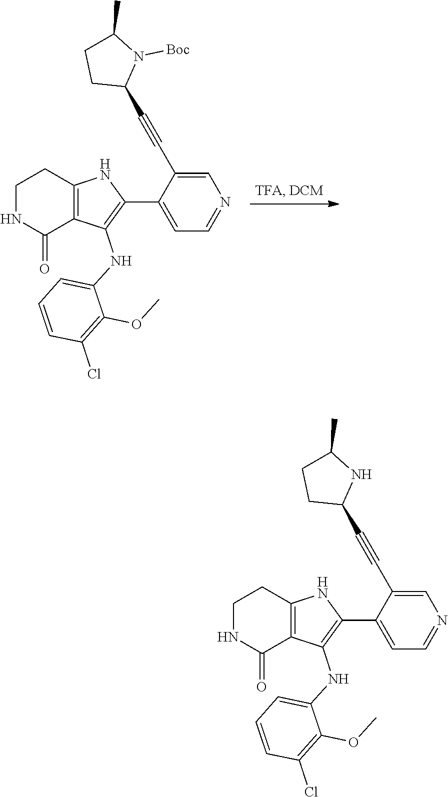 [SMILES: COc1c(Cl)cccc1Nc1c(-c2ccncc2C#C[C@H]2CC[C@@H](C)N2C(=O)OC(C)(C)C)[nH]c2c1C(=O)NCC2 ; COc1c(Cl)cccc1Nc1c(-c2ccncc2C#C[C@H]2CC[C@@H](C)N2)[nH]c2c1C(=O)NCC2]

To a stirred solution of tert-butyl (2R,5R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate (90.0 mg, 0.156 mmol, 1 equiv) in DCM (3 mL) was added TFA (1 mL) at room temperature under nitrogen atmosphere. The resulting mixture was stirred for 1 h and concentrated under reduced pressure to afford 3-[(3-chloro-2-methoxyphenyl)amino]-2-(3-{2-[(2R,5R)-5-methylpyrrolidin-2-yl]ethynyl}pyridin-4-yl)-1H,5H,6H,7H-pyrrolo[3,2-c]pyridin-4-one (120 mg, crude) as a yellow oil.

LC-MS: (M+H)⁺ found 476.0

### 66.6. Synthesis of 3-[(3-chloro-2-methoxyphenyl)amino]-2-(3-{2-[(2R,5R)-5-methyl-1-(prop-2-enoyl)pyrrolidin-2-yl]ethynyl}pyridin-4-yl)-1H,5H,6H,7H-pyrrolo[3,2-c]pyridin-4-one

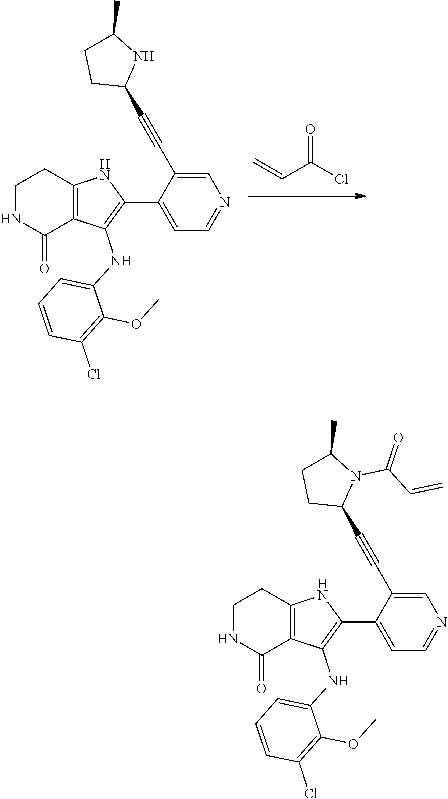

A mixture of 3-[(3-chloro-2-methoxyphenyl)amino]-2-(3-{2-[(2R,5R)-5-methylpyrrolidin-2-yl]ethynyl}pyridin-4-yl)-1H,5H,6H,7H-pyrrolo[3,2-c]pyridin-4-one (120 mg, 0.252 mmol, 1 equiv) in THF (2 mL) was basified to pH 8 with saturated NaHCO₃(aq.). To the above mixture was added acryloyl chloride (41.1 mg, 0.454 mmol, 1.8 equiv) dropwise at 0° C. The resulting mixture was stirred for 30 min at room temperature. The reaction was quenched with MeOH (0.5 mL) at 0° C. The mixture was extracted with CH2Cl2/MeOH (10/1) (2×10 mL), dried over anhydrous Na₂SO₄ and filtered. The filtrate was concentrated under reduced pressure to give crude product (100 mg) that was purified by Prep-HPLC under the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 33% B to 49% B in 8 min, 49% B; Wave Length: 254/220 nm; RT1(min): 8;) to afford 3-[(3-chloro-2-methoxyphenyl)amino]-2-(3-{2-[(2R,5R)-5-methyl-1-(prop-2-enoyl)pyrrolidin-2-yl]ethynyl}pyridin-4-yl)-1H,5H,6H,7H-pyrrolo[3,2-c]pyridin-4-one (28.7 mg, 21.1%) as a yellow solid.

LC-MS: (M+H)⁺ found 530.10

¹H NMR (400 MHz, Chloroform-d) δ 11.25 (s, 1H), 8.56 (s, 1H), 8.13 (s, 1H), 7.80 (s, 1H), 7.44 (s, 1H), 6.76-6.69 (m, 1H), 6.61 (t, 1H), 6.49-6.57 (m, 1H), 6.47-6.39 (m, 1H), 6.22 (d, 1H), 5.82-5.75 (m, 1H), 5.22 (s, 1H), 4.83 (t, 1H), 4.33-4.21 (m, 1H), 4.07 (s, 3H), 3.66-3.56 (m, 2H), 3.34-3.19 (m, 2H), 2.52-2.29 (m, 2H), 2.23-2.09 (m, 1H), 1.97-1.88 (m, 1H), 1.42 (d, 3H).

### Example 67. 3-[(3-chloro-2-methoxyphenyl)amino]-2-(3-{2-[(1S,3R,4R)-2-(prop-2-enoyl)-2-azabicyclo[2.2.1]heptan-3-yl]ethynyl}pyridin-4-yl)-1H,5H,6H,7H-pyrrolo[3,2-c]pyridin-4-one (compound 236)

### 67.1. Synthesis of tert-butyl (1S,3R,4R)-3-(hydroxymethyl)-2-azabicyclo[2.2.1]heptane-2-carboxylate

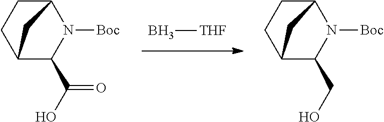

To a stirred solution of (1S,3R,4R)-2-(tert-butoxycarbonyl)-2-azabicyclo[2.2.1]heptane-3-carboxylic acid (1 g, 4.14 mmol, 1 equiv) in THF (10 mL) was added BH₃-THF (0.43 g, 4.97 mmol, 1.2 equiv) dropwise at 0° C. under N₂ atmosphere. The resulting mixture was stirred for 2 h at room temperature under N₂ atmosphere. The reaction was quenched by the addition of saturated aqueous NaHCO₃ at 0° C. and extracted with EA (50 mL). The organic layer was washed with saturated aqueous NaCl (50 mL) and concentrated under reduced pressure. This resulted in tert-butyl (1S,3R,4R)-3-(hydroxymethyl)-2-azabicyclo[2.2.1]heptane-2-carboxylate (0.94 g, crude) as a colourless oil.

### 67.2. Synthesis of tert-butyl (1S,3R,4R)-3-formyl-2-azabicyclo[2.2.1]heptane-2-carboxylate

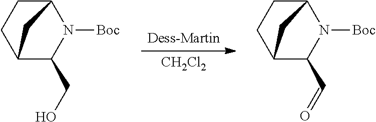 [SMILES: CC(C)(C)OC(=O)N1[C@H]2CC[C@H](C2)[C@@H]1CO ; "CH2Cl2" ; CC(C)(C)OC(=O)N1[C@H]2CC[C@H](C2)[C@@H]1C=O]

To a stirred solution of tert-butyl (1S,3R,4R)-3-(hydroxymethyl)-2-azabicyclo[2.2.1]heptane-2-carboxylate (0.94 g, 4.13 mmol, 1 equiv) in methylene chloride (10 mL) was added Dess-Martin (1.93 g, 4.54 mmol, 1.1 equiv) at 0° C. under N₂ atmosphere. The resulting mixture was stirred for 2 h at room temperature under N₂ atmosphere. The reaction was quenched by the addition of saturated aqueous NaHCO₃ at 0° C. and extracted with DCM (10 mL). The organic layer was washed with saturated aqueous NaCl (20 mL) and concentrated under reduced pressure. The residue was purified by silica gel column chromatography, eluted with PE/EA=8:1 to afford tert-butyl (1S,3R,4R)-3-formyl-2-azabicyclo[2.2.1]heptane-2-carboxylate (879 mg, 94.3%) as a colourless oil.

### 67.3. Synthesis of tert-butyl (1S,3R,4R)-3-ethynyl-2-azabicyclo[2.2.1]heptane-2-carboxylate

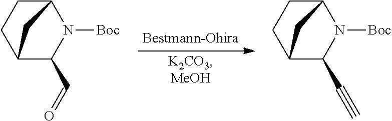

To a stirred solution of tert-butyl (1S,3R,4R)-3-formyl-2-azabicyclo[2.2.1]heptane-2-carboxylate (800 mg, 3.55 mmol, 1 equiv) and K₂CO₃ (981 mg, 7.10 mmol, 2 equiv) in MeOH (20 mL) was added dimethyl (1-diazo-2-oxopropyl)phosphonate (818 mg, 4.26 mmol, 1.2 equiv) dropwise at 0° C. under N₂ atmosphere. The resulting mixture was stirred for 1 h at room temperature under N₂ atmosphere. The reaction was quenched by the addition of potassium sodium tartrate and saturated aqueous NaHCO₃ (2 mL). The mixture was extracted with EA (20 mL) and concentrated under reduced pressure. The residue was purified by silica gel column chromatography, eluted with PE/EA=2:1 to afford tert-butyl (1S,3R,4R)-3-ethynyl-2-azabicyclo[2.2.1]heptane-2-carboxylate (625 mg, 79.5%) as a white solid.

### 67.4. Synthesis of tert-butyl (1S,3R,4R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[2.2.1]heptane-2-carboxylate

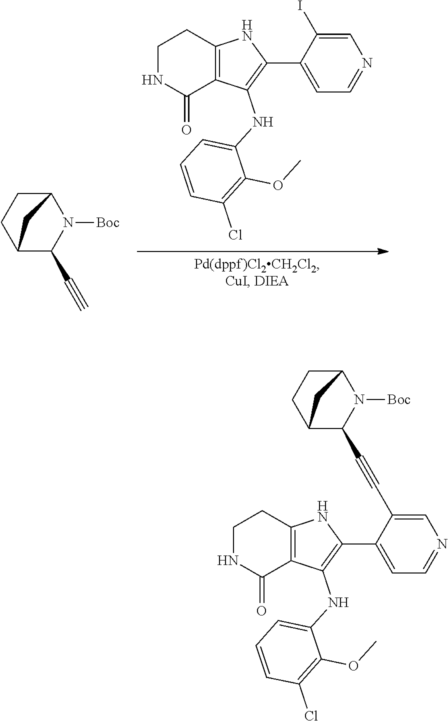 [SMILES: C#C[C@H]1[C@@H]2CC[C@@H](C2)N1C(=O)OC(C)(C)C ; COc1c(Cl)cccc1Nc1c(-c2ccncc2I)[nH]c2c1C(=O)NCC2 ; COc1c(Cl)cccc1Nc1c(-c2ccncc2C#C[C@H]2[C@@H]3CC[C@@H](C3)N2C(=O)OC(C)(C)C)[nH]c2c1C(=O)NCC2]

To a stirred solution of tert-butyl (1S,3R,4R)-3-ethynyl-2-azabicyclo[2.2.1]heptane-2-carboxylate (134 mg, 0.606 mmol, 3 equiv) and 3-[(3-chloro-2-methoxyphenyl)amino]-2-(3-iodopyridin-4-yl)-1H,5H,6H,7H-pyrrolo[3,2-c]pyridin-4-one (100 mg, 0.202 mmol, 1.00 equiv) and CuI (19 mg, 0.101 mmol, 0.5 equiv) and 1,1′-bis(diphenylphosphino)ferrocene-palladium(II)dichloride dichloromethane complex (41 mg, 0.051 mmol, 0.25 equiv) in DMF (2.5 mL) were added DIEA (130 mg, 1.01 mmol, 5 equiv) under argon atmosphere. The resulting suspension was backfilled with argon three times and stirred for 2 h at 50° C. The resulting mixture was concentrated under reduced pressure. The residue was purified by reverse flash chromatography with the following conditions (column, C18 silica gel; mobile phase, MeCN in water, 60% to 70% gradient in 10 min; detector, UV 254 nm). This resulted in tert-butyl (1S,3R,4R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[2.2.1]heptane-2-carboxylate (101 mg, 84.9%) as a yellow solid.

LC-MS: (M+H)⁺ found 588.

### 67.5. Synthesis of 2-(3-{2-[(1S,3R,4R)-2-azabicyclo[2.2.1]heptan-3-yl]ethynyl}pyridin-4-yl)-3-[(3-chloro-2-methoxyphenyl)amino]-1H,5H,6H,7H-pyrrolo[3,2-c]pyridin-4-one

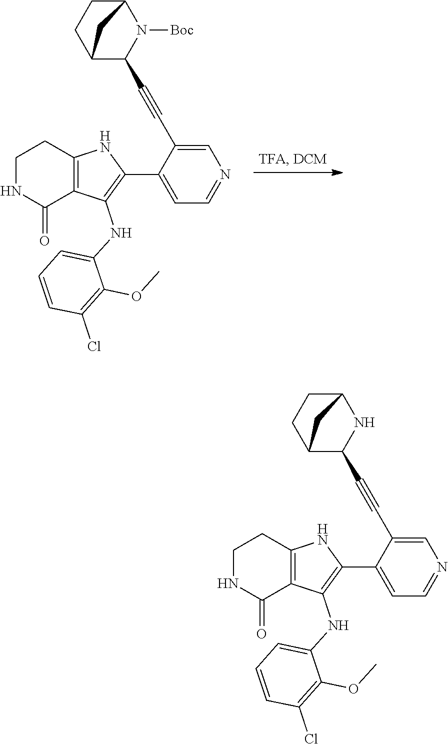 [SMILES: COc1c(Cl)cccc1Nc1c(-c2ccncc2C#C[C@H]2[C@@H]3CC[C@@H](C3)N2C(=O)OC(C)(C)C)[nH]c2c1C(=O)NCC2 ; COc1c(Cl)cccc1Nc1c(-c2ccncc2C#C[C@@H]2N[C@H]3CC[C@@H]2C3)[nH]c2c1C(=O)NCC2]

To a stirred solution of tert-butyl (1S,3R,4R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[2.2.1]heptane-2-carboxylate (100 mg, 0.170 mmol, 1 equiv) in DCM (2 mL) was added TFA (0.7 mL) at room temperature under nitrogen atmosphere. The resulting mixture was stirred for 1 h and concentrated under reduced pressure to afford 2-(3-{2-[(1S,3R,4R)-2-azabicyclo[2.2.1]heptan-3-yl]ethynyl}pyridin-4-yl)-3-[(3-chloro-2-methoxyphenyl)amino]-1H,5H,6H,7H-pyrrolo[3,2-c]pyridin-4-one (50 mg, crude) as a red oil.

LC-MS: (M+H)⁺ found 488.

### 67.6. Synthesis of 3-[(3-chloro-2-methoxyphenyl)amino]-2-(3-{2-[(1S,3R,4R)-2-(prop-2-enoyl)-2-azabicyclo[2.2.1]heptan-3-yl]ethynyl}pyridin-4-yl)-1H,5H,6H,7H-pyrrolo[3,2-c]pyridin-4-one

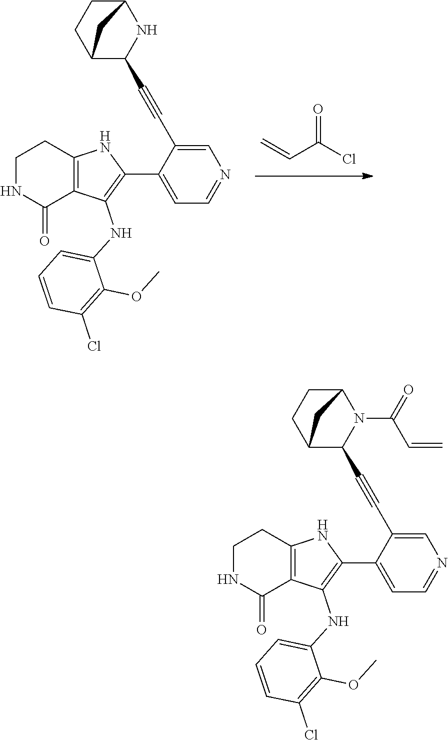

A mixture of 2-(3-{2-[(1S,3R,4R)-2-azabicyclo[2.2.1]heptan-3-yl]ethynyl}pyridin-4-yl)-3-[(3-chloro-2-methoxyphenyl)amino]-1H,5H,6H,7H-pyrrolo[3,2-c]pyridin-4-one (50 mg, 0.102 mmol, 1 equiv) in THF (2 mL) was basified to pH 8 with NaHCO₃saturated solution. To the above mixture was added acryloyl chloride (8.35 mg, 0.092 mmol, 0.9 equiv) dropwise at 0° C. The resulting mixture was stirred for 2 h at room temperature. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (130 mg) that was purified by Prep-HPLC under the following conditions (Column: Aeris PEPTIDE Sum XB-C18 Axia, 21.2 mm×250 mm, 5 μm; Mobile Phase A: THF—HPLC, Mobile Phase B: Hex—HPLC; Flow rate: 25 mL/min; Gradient: 32% B to 62% B in 10 min, 62% B; Wave Length: 254/220 nm; RT1(min): 9.32; Number Of Runs: 0). This resulted in 3-[(3-chloro-2-methoxyphenyl)amino]-2-(3-{2-[(1S,3R,4R)-2-(prop-2-enoyl)-2-azabicyclo[2.2.1]heptan-3-yl]ethynyl}pyridin-4-yl)-1H,5H,6H,7H-pyrrolo[3,2-c]pyridin-4-one (25 mg, 43.7%) as a yellow solid

LC-MS: (M+H)⁺ found 542.40.

¹H NMR (400 MHz, Chloroform-d) δ 11.02 (s, 1H), 8.53 (s, 1H), 8.08 (s, 1H), 7.91 (s, 1H), 7.41 (s, 1H), 6.79-6.71 (m, 1H), 6.62 (t, 1H), 6.57-6.48 (m, 1H), 6.43-6.34 (m, 1H), 6.24-6.15 (m, 1H), 5.81-5.72 (m, 1H), 5.63-5.52 (m, 1H), 5.28 (s, 1H), 4.48 (s, 1H), 4.43 (s, 1H), 4.07 (s, 3H), 4.00-3.91 (m, 2H), 3.70-3.56 (m, 2H), 3.37-3.23 (m, 2H), 2.87 (s, 1H), 2.31-2.22 (m, 1H), 2.10-1.50 (m, 2H).

### Example 68. 3-[(3-chloro-2-methoxyphenyl)amino]-2-(3-{2-[(2R)-4,4-dimethyl-1-(prop-2-enoyl)pyrrolidin-2-yl]ethynyl}pyridin-4-yl)-1H,5H,6H,7H-pyrrolo[3,2-c]pyridin-4-one (compound 235)

### 68.1. Synthesis of tert-butyl (2R)-2-(hydroxymethyl)-4,4-dimethylpyrrolidine-1-carboxylate

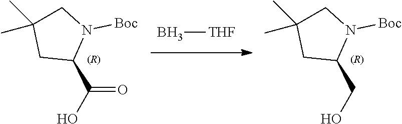

To a stirred solution of (2R)-1-(tert-butoxycarbonyl)-4,4-dimethylpyrrolidine-2-carboxylic acid (1.00 g, 4.11 mmol, 1.00 equiv) in THF (10.0 ml) was added BH₃-THF (423 mg, 4.93 mmol, 1.02 equiv) at 0 degrees C. under N₂ atmosphere. The resulting mixture was stirred for 1 h at 25 degrees C. The resulting solution was quenched by the addition of MeOH (10 mL). The mixture was concentrated under vacuum to afford tert-butyl (2R)-2-(hydroxymethyl)-4,4-dimethylpyrrolidine-1-carboxylate (1.01 g, crude) as a colorless oil.

LC-MS: M+H-56 found: 174.00.

### 68.2. Synthesis of tert-butyl (2R)-2-formyl-4,4-dimethylpyrrolidine-1-carboxylate

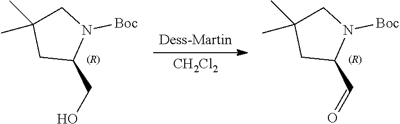

To a stirred solution of tert-butyl (2R)-2-(hydroxymethyl)-4,4-dimethylpyrrolidine-1-carboxylate (700 mg, 3.05 mmol, 1.00 equiv) in DCM (7.00 mL) was added Dess-Martin (1.94 g, 4.57 mmol, 1.50 equiv) at 0 degrees C. under N₂ atmosphere. The resulting mixture was stirred for 1 h at 25 degrees C. The resulting solution was quenched by the addition of Na₂SO₃. The mixture was neutralized to pH 7 with Na₂CO₃ and extracted with DCM (3×20 mL). The combined organic layers were washed with NaCl (3×50 mL), dried over anhydrous Na₂SO₄ and concentrated under vacuum. The residue was purified by silica gel column chromatography, eluted with PE/EA (20/1) to afford tert-butyl (2R)-2-formyl-4,4-dimethylpyrrolidine-1-carboxylate (600 mg, 86.5%) as a colorless oil.

### 68.3. Synthesis of tert-butyl (2R)-2-ethynyl-4,4-dimethylpyrrolidine-1-carboxylate

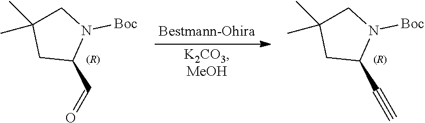

To a stirred solution of tert-butyl (2R)-2-formyl-4,4-dimethylpyrrolidine-1-carboxylate (540 mg, 2.37 mmol, 1.00 equiv) and K₂CO₃ (656 mg, 4.75 mmol, 2.00 equiv) in MeOH (2 mL) were added Bestmann-Ohira reagent (547 mg, 2.85 mmol, 1.20 equiv) at 0 degrees C. under N₂ atmosphere. The resulting mixture was stirred for 2 h at 25 degrees C. The resulting solution was quenched by the addition of Rochelle salt. The mixture was extracted with EA (3×20 mL). The combined organic layers were washed with brine (3×50 mL), dried over anhydrous Na₂SO₄ and concentrated under vacuum. The residue was purified by silica gel column chromatography, eluted with PE/EA (50/1) to afford tert-butyl (2R)-2-ethynyl-4,4-dimethylpyrrolidine-1-carboxylate (440 mg, 74.64%) as a colorless oil.

### 68.4. Synthesis of tert-butyl (2R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-4,4-dimethylpyrrolidine-1-carboxylate

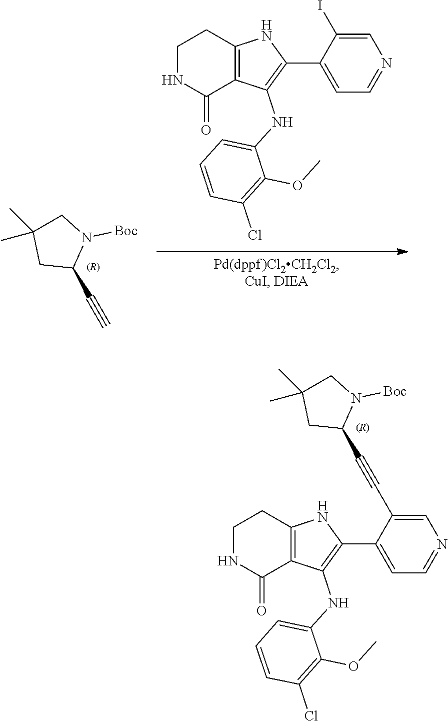

To a stirred mixture of 3-[(3-chloro-2-methoxyphenyl)amino]-2-(3-iodopyridin-4-yl)-1H,5H,6H,7H-pyrrolo[3,2-c]pyridin-4-one (200 mg, 0.40 mmol, 1.00 equiv), CuI (38 mg, 0.20 mmol, 0.5 equiv) and Pd(dppf)Cl2CH2Cl2 (91 mg, 0.11 mmol, 0.25 equiv) in DMF (4 mL) were added tert-butyl (2R)-2-ethynyl-4,4-dimethylpyrrolidine-1-carboxylate (90 mg, 0.40 mmol, 1.00 equiv) and DIEA (261 mg, 2.020 mmol, 5 equiv) under argon atmosphere. The resulting suspension was backfilled with argon three times and stirred for 2 h at 50° C. Desired product could be detected by LCMS. The precipitated solids were collected by filtration and washed with H₂O (3×10 mL). The residue was purified by silica gel column chromatography, eluted with PE/EA (1/1) to afford tert-butyl (2R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-4,4-dimethylpyrrolidine-1-carboxylate (130 mg, 53.95%) as a yellow solid.

LC-MS: M+H found: 590.15.

### 68.5. Synthesis of 3-[(3-chloro-2-methoxyphenyl) amino]-2-(3-{2-[(2R)-4,4-dimethylpyrrolidin-2-yl] ethynyl} pyridin-4-yl)-1H,5H,6H,7H-pyrrolo[3,2-c]pyridin-4-one

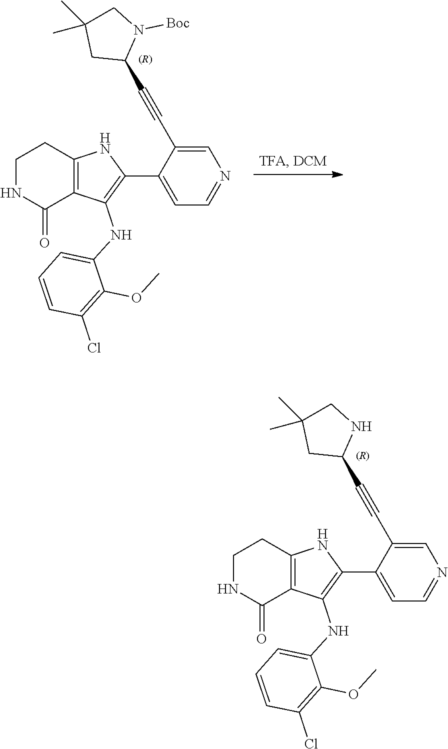

To a solution of tert-butyl (2S)-2-[2-(4-{3-[(3-chloro-2-methoxyphenyl) amino]-4-oxo-1H,5H,6H,7H-pyrrolo[3,2-c] pyridin-2-yl} pyridin-3-yl) ethynyl]-2-methylpyrrolidine-1-carboxylate (60 mg, 0.10 mmol, 1.00 equiv) in DCM (1.5 mL) was added TFA (0.5 mL) at room temperature under nitrogen atmosphere. The resulting mixture was stirred for 1 h and concentrated under reduced pressure to afford 3-[(3-chloro-2-methoxyphenyl) amino]-2-(3-{2-[(2R)-4,4-dimethylpyrrolidin-2-yl] ethynyl} pyridin-4-yl)-1H,5H,6H,7H-pyrrolo[3,2-c] pyridin-4-one (70 mg, crude) as a red oil.

LC-MS: M+H found: 490.20.

### 68.6. Synthesis of 3-[(3-chloro-2-methoxyphenyl)amino]-2-(3-{2-[(2R)-4,4-dimethyl-1-(prop-2-enoyl)pyrrolidin-2-yl]ethynyl}pyridin-4-yl)-1H,5H,6H,7H-pyrrolo[3,2-c]pyridin-4-one

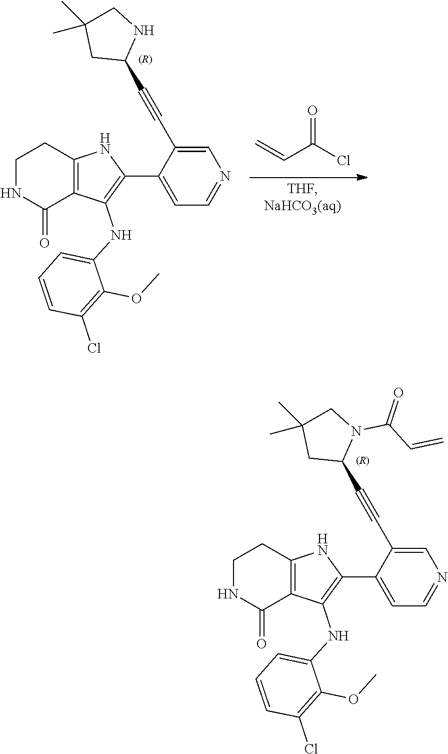

To a stirred solution of 3-[(3-chloro-2-methoxyphenyl) amino]-2-(3-{2-[(2R)-4,4-dimethylpyrrolidin-2-yl] ethynyl} pyridin-4-yl)-1H,5H,6H,7H-pyrrolo[3,2-c] pyridin-4-one (70 mg, 0.14 mmol, 1.00 equiv) in THF (3 mL) was basified to pH 8 with sat. NaHCO₃(aq.). To the above mixture was added was added acryloyl chloride (14 mg, 0.15 mmol, 1.10 equiv) dropwise at 0° C. under nitrogen atmosphere. The resulting mixture was stirred for 2 h at room temperature under nitrogen atmosphere. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (50 mg) that was purified by Prep-HPLC under the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 33% B to 56% B in 8 min, 56% B; Wave Length: 254/220 nm; RT1(min): 8; Number Of Runs: 0) to afford 3-[(3-chloro-2-methoxyphenyl)amino]-2-(3-{2-[(2R)-4,4-dimethyl-1-(prop-2-enoyl)pyrrolidin-2-yl]ethynyl}pyridin-4-yl)-1H,5H,6H,7H-pyrrolo[3,2-c]pyridin-4-one (22.3 mg, 28.41%) as a yellow solid.

LC-MS: (M+H)⁺ found 544.50.

¹H NMR (400 MHz, Chloroform-d) δ 11.21 (s, 1H), 8.54 (s, 1H), 8.13 (s, 1H), 7.74 (s, 1H), 7.47-7.36 (m, 1H), 6.71-6.61 (m, 1H), 6.60 (t, 1H), 6.51-6.36 (m, 2H), 6.22-6.10 (m, 1H), 5.76-5.65 (m, 1H), 5.34 (s, 1H), 4.93 (t, 1H), 4.07 (s, 3H), 3.67-3.56 (m, 2H), 3.55-3.41 (m, 2H), 3.40-3.21 (m, 2H), 2.25-2.18 (m, 1H), 2.12-2.06 (m, 1H), 1.33 (s, 3H), 1.15 (s, 3H).

### Example 69. 3-[(3-fluoro-2-methoxyphenyl)amino]-2-(3-{2-[(2R,5R)-5-methyl-1-(prop-2-enoyl)pyrrolidin-2-yl]ethynyl}pyridin-4-yl)-1H,5H,6H,7H-pyrrolo[3,2-c]pyridin-4-one (compound 256)

### 69.1. Synthesis of tert-butyl (2R,5R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate

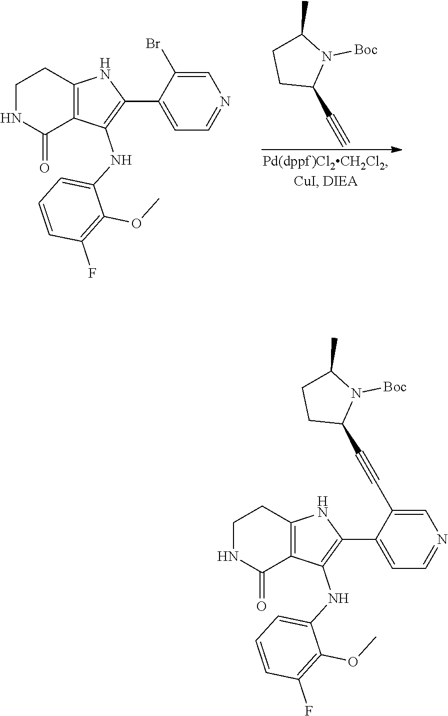 [SMILES: COc1c(F)cccc1Nc1c(-c2ccncc2Br)[nH]c2c1C(=O)NCC2 ; C#C[C@H]1CC[C@@H](C)N1C(=O)OC(C)(C)C ; COc1c(F)cccc1Nc1c(-c2ccncc2C#C[C@H]2CC[C@@H](C)N2C(=O)OC(C)(C)C)[nH]c2c1C(=O)NCC2]

A mixture of 2-(3-bromopyridin-4-yl)-3-[(3-fluoro-2-methoxyphenyl)amino]-1H,5H,6H,7H-pyrrolo[3,2-c]pyridin-4-one (100 mg, 0.232 mmol, 1 equiv), tert-butyl (2R,5R)-2-ethynyl-5-methylpyrrolidine-1-carboxylate (97.1 mg, 0.464 mmol, 2 equiv), Pd(dppf)Cl₂·CH₂Cl₂ (47.2 mg, 0.058 mmol, 0.25 equiv), CuI (22.1 mg, 0.116 mmol, 0.5 equiv) and DIEA (149 mg, 1.160 mmol, 5 equiv) in DMF (2 mL) at room temperature under argon atmosphere. The resulting mixture was stirred overnight at 50° C. under argon atmosphere. The resulting mixture was concentrated under vacuum. The residue was purified by reverse flash chromatography with the following conditions: column, C18 silica gel; mobile phase, MeCN in water, 10% to 80% gradient in 20 min; detector, UV 254 nm. This resulted in tert-butyl (2R,5R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate (100 mg, 77.0%) as a yellow solid.

LC-MS: (M+H)⁺ found 560.35

### 69.2. Synthesis of (3-[(3-fluoro-2-methoxyphenyl)amino]-2-(3-{2-[(2R,5R)-5-methylpyrrolidin-2-yl]ethynyl}pyridin-4-yl)-1H,5H,6H,7H-pyrrolo[3,2-c]pyridin-4-one

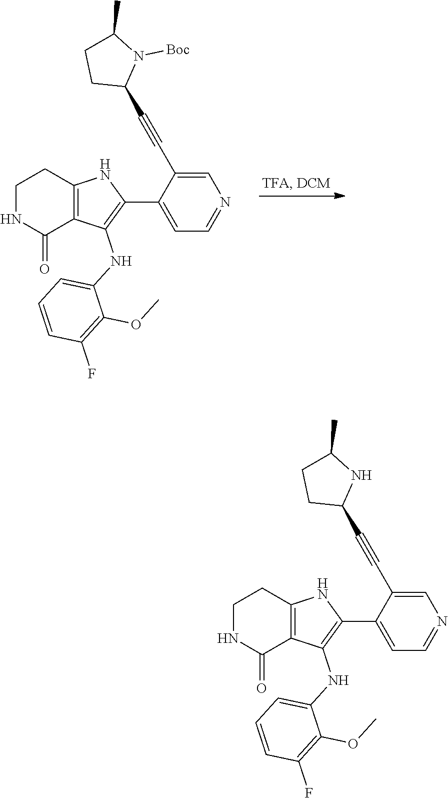 [SMILES: COc1c(F)cccc1Nc1c(-c2ccncc2C#C[C@H]2CC[C@@H](C)N2C(=O)OC(C)(C)C)[nH]c2c1C(=O)NCC2 ; COc1c(F)cccc1Nc1c(-c2ccncc2C#C[C@H]2CC[C@@H](C)N2)[nH]c2c1C(=O)NCC2]

To a stirred solution of tert-butyl (2R,5R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate (100 mg, 0.179 mmol, 1 equiv) in DCM (3 mL) was added TFA (1 mL) at room temperature under nitrogen atmosphere. The resulting mixture was stirred for 1 h and concentrated under reduced pressure to afford (3-[(3-fluoro-2-methoxyphenyl)amino]-2-(3-{2-[(2R,5R)-5-methylpyrrolidin-2-yl]ethynyl}pyridin-4-yl)-1H,5H,6H,7H-pyrrolo[3,2-c]pyridin-4-one (100 mg, crude) as a yellow oil.

LC-MS: (M+H)⁺ found 460.1

### 69.3. Synthesis of 3-[(3-fluoro-2-methoxyphenyl)amino]-2-(3-{2-[(2R,5R)-5-methyl-1-(prop-2-enoyl)pyrrolidin-2-yl]ethynyl}pyridin-4-yl)-1H,5H,6H,7H-pyrrolo[3,2-c]pyridin-4-one

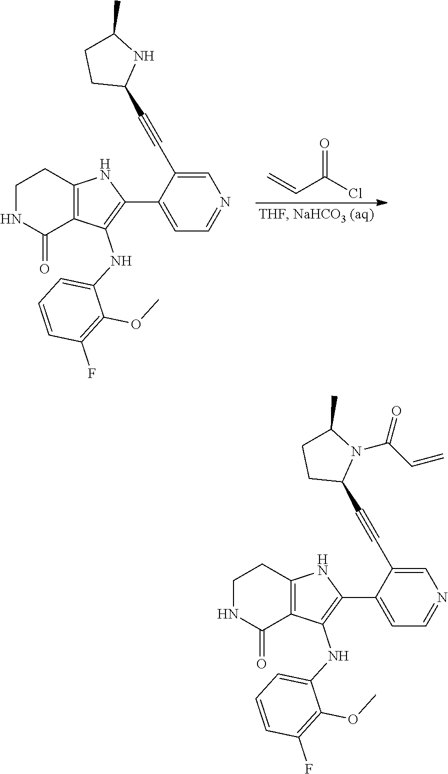

A mixture of 3-[(3-fluoro-2-methoxyphenyl)amino]-2-(3-{2-[(2R,5R)-5-methylpyrrolidin-2-yl]ethynyl}pyridin-4-yl)-1H,5H,6H,7H-pyrrolo[3,2-c]pyridin-4-one (100 mg, 0.218 mmol, 1 equiv) in THF (2 mL) was basified to pH 8 with saturated NaHCO₃(aq.). To the above mixture was added acryloyl chloride (29.5 mg, 0.327 mmol, 1.5 equiv) dropwise at 0° C. The resulting mixture was stirred for 30 min at 0° C. The reaction was quenched with MeOH (0.5 mL) at 0° C. The mixture was extracted with CH2Cl2/MeOH 10:1 (2×10 mL), dried over anhydrous Na₂SO₄ and filtered. The filtrate was concentrated under reduced pressure to give crude product (110 mg) that was purified by Prep-HPLC under the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 58% B in 7 min; Wave Length: 254 nm; RT1(min): 6.5;) to afford 3-[(3-fluoro-2-methoxyphenyl)amino]-2-(3-{2-[(2R,5R)-5-methyl-1-(prop-2-enoyl)pyrrolidin-2-yl]ethynyl}pyridin-4-yl)-1H,5H,6H,7H-pyrrolo[3,2-c]pyridin-4-one (31.5 mg, 28.1%) as a yellow solid.

LC-MS: (M+H)⁺ found 514.15.

¹H NMR (400 MHz, Chloroform-d) δ 11.26 (s, 1H), 8.54 (s, 1H), 8.11 (d, 1H), 7.84 (s, 1H), 7.47 (d, 1H), 6.63-6.38 (m, 4H), 6.09-6.07 (m, 1H), 5.80-5.77 (m, 1H), 5.22 (s, 1H), 4.84-4.80 (m, 1H), 4.26-4.25 (m, 1H), 4.10 (d, 3H), 3.62-3.58 (m, 2H), 3.33-3.24 (m, 2H), 2.53-2.41 (m, 1H), 2.41-2.30 (m, 1H), 2.16 (t, 1H), 1.95-1.93 (m, 1H), 1.42 (d, 3H).

### Example 70. 3-[(3-fluoro-2-methoxyphenyl)amino]-2-(3-{2-[(2S,5S)-5-methyl-1-(prop-2-enoyl)pyrrolidin-2-yl]ethynyl}pyridin-4-yl)-1H,5H,6H,7H-pyrrolo[3,2-c]pyridin-4-one (compound 255)

### 70.1. Synthesis of -(3-(((2S,5S)-1-acryloyl-5-methylpyrrolidin-2-yl)ethynyl)pyridin-4-yl)-3-((3-fluoro-2-methoxyphenyl)amino)-1,5,6,7-tetrahydro-4H-pyrrolo[3,2-c]pyridin-4-one

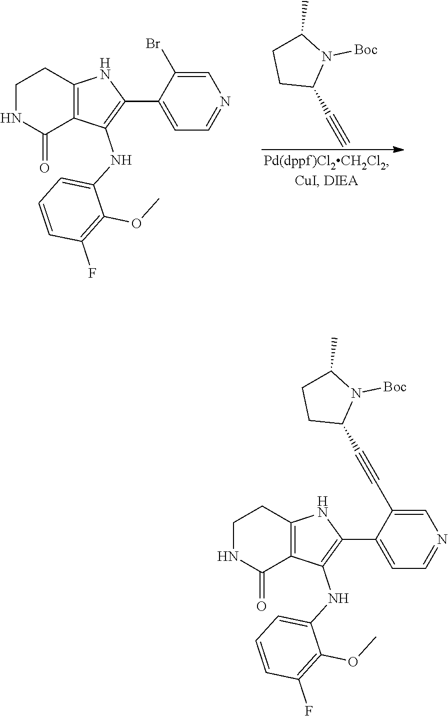 [SMILES: COc1c(F)cccc1Nc1c(-c2ccncc2Br)[nH]c2c1C(=O)NCC2 ; C#C[C@@H]1CC[C@H](C)N1C(=O)OC(C)(C)C ; COc1c(F)cccc1Nc1c(-c2ccncc2C#C[C@@H]2CC[C@H](C)N2C(=O)OC(C)(C)C)[nH]c2c1C(=O)NCC2]

To a stirred mixture of 2-(3-bromopyridin-4-yl)-3-((3-fluoro-2-methoxyphenyl)amino)-1,5,6,7-tetrahydro-4H-pyrrolo[3,2-c]pyridin-4-one (200 mg, 0.47 mmol, 1.00 equiv) and Pd(dppf)Cl₂CH₂Cl₂ (95 mg, 0.11 mmol, 0.25 equiv) in DMF (4 mL) were added tert-butyl (2S,5S)-2-ethynyl-5-methylpyrrolidine-1-carboxylate (486 mg, 2.25 mmol, 5.00 equiv), CuI (44 mg, 0.23 mmol, 0.50 equiv) and DIEA (300 mg, 2.25 mmol, 5.00 equiv) under argon atmosphere. The resulting suspension was backfilled with argon three times and stirred for overnight at 50° C. The resulting mixture was concentrated under reduced pressure. The residue was purified by reverse flash chromatography with the following conditions: column, silica gel; mobile phase, MeCN in water, 20% to 100% gradient in min; detector, UV 254 nm to afford 2-(3-(((2S,5S)-1-acryloyl-5-methylpyrrolidin-2-yl)ethynyl)pyridin-4-yl)-3-((3-fluoro-2-methoxyphenyl)amino)-1,5,6,7-tetrahydro-4H-pyrrolo[3,2-c]pyridin-4-one (260 mg, 86%) as a yellow solid.

LC-MS: (M+H)⁺ found 560.50.

### 70.2. Synthesis of 3-[(3-fluoro-2-methoxyphenyl)amino]-2-(3-{2-[(2S,5S)-5-methylpyrrolidin-2-yl]ethynyl}pyridin-4-yl)-1H,5H,6H,7H-pyrrolo[3,2-c]pyridin-4-one

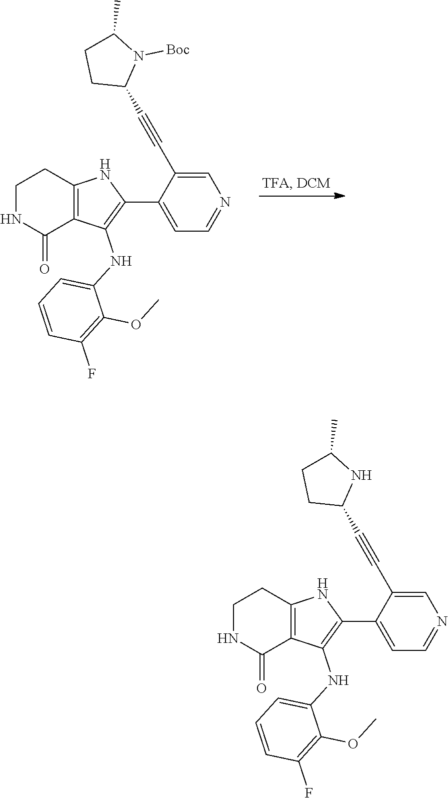 [SMILES: COc1c(F)cccc1Nc1c(-c2ccncc2C#C[C@@H]2CC[C@H](C)N2C(=O)OC(C)(C)C)[nH]c2c1C(=O)NCC2 ; COc1c(F)cccc1Nc1c(-c2ccncc2C#C[C@@H]2CC[C@H](C)N2)[nH]c2c1C(=O)NCC2]

To a stirred solution of tert-butyl (2S,5S)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-methylpyrrolidine-1-carboxylate (23 mg, 0.041 mmol, 1 equiv) in DCM (0.9 mL) was added TFA (0.3 mL) at room temperature under nitrogen atmosphere. The resulting mixture was stirred for 1 h and concentrated under reduced pressure to afford 3-[(3-fluoro-2-methoxyphenyl)amino]-2-(3-{2-[(2S,5S)-5-methylpyrrolidin-2-yl]ethynyl}pyridin-4-yl)-1H,5H,6H,7H-pyrrolo[3,2-c]pyridin-4-one (80 mg, crude) as a red oil.

LC-MS: (M+H)⁺ found 460.10.

### 70.3. Synthesis of 3-[(3-fluoro-2-methoxyphenyl)amino]-2-(3-{2-[(2S,5S)-5-methyl-1-(prop-2-enoyl)pyrrolidin-2-yl]ethynyl}pyridin-4-yl)-1H,5H,6H,7H-pyrrolo[3,2-c]pyridin-4-one

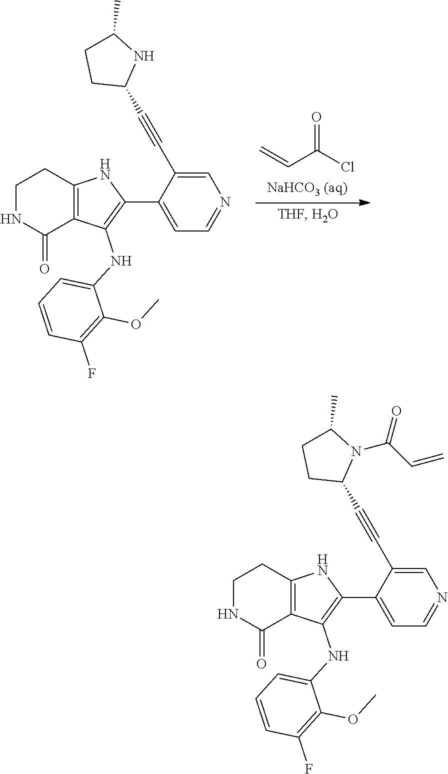

To a stirred solution of 3-[(3-fluoro-2-methoxyphenyl)amino]-2-(3-{2-[(2S,5S)-5-methylpyrrolidin-2-yl]ethynyl}pyridin-4-yl)-1H,5H,6H,7H-pyrrolo[3,2-c]pyridin-4-one (82 mg, 0.178 mmol, 1 equiv) in THF (2 mL) was basified to pH 8 with sat. NaHCO₃(aq.). To the above mixture was added acryloyl chloride (16.2 mg, 0.178 mmol, 1.00 equiv) was slowly added to the solution. The resulting mixture was stirred for 0.5 h at 0° C. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (120 mg) that was purified by Prep-HPLC under the following conditions (Column: X Bridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 38% B to 52% B in 10 min, 52% B; Wave Length: 254/220 nm; RT1(min): 8.85; Number Of Runs: 0) to afford 3-[(3-fluoro-2-methoxyphenyl)amino]-2-(3-{2-[(2S,5S)-5-methyl-1-(prop-2-enoyl)pyrrolidin-2-yl]ethynyl}pyridin-4-yl)-1H,5H,6H,7H-pyrrolo[3,2-c]pyridin-4-one (36.6 mg, 39.62%) as a yellow solid.

LC-MS: (M+H)⁺ found 514.15.

¹H NMR (400 MHz, Chloroform-d) δ 11.12 (s, 1H), 8.60-7.9 (m, 2H), 7.64 (s, 1H), 7.58-7.34 (m, 1H), 6.58-6.26 (m, 4H), 6.02 (d, 1H), 5.78-5.65 (m, 1H), 5.19 (s, 1H), 4.77 (t, 1H), 4.34-4.11 (m, 1H), 4.03 (s, 3H), 3.65-3.42 (m, 2H), 3.33-3.06 (m, 2H), 2.52-2.20 (m, 2H), 2.18-1.99 (m, 1H), 1.92-1.79 (m, 1H), 1.35 (d, 3H).

### Example 71. 3-[(3-fluoro-2-methoxyphenyl)amino]-2-(3-{2-[(1S,3R,4R)-2-(prop-2-enoyl)-2-azabicyclo[2.2.1]heptan-3-yl]ethynyl}pyridin-4-yl)-1H,5H,6H,7H-pyrrolo[3,2-c]pyridin-4-one (compound 252)

### 71.1. Synthesis of tert-butyl (1S,3R,4R)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[2.2.1]heptane-2-carboxylate

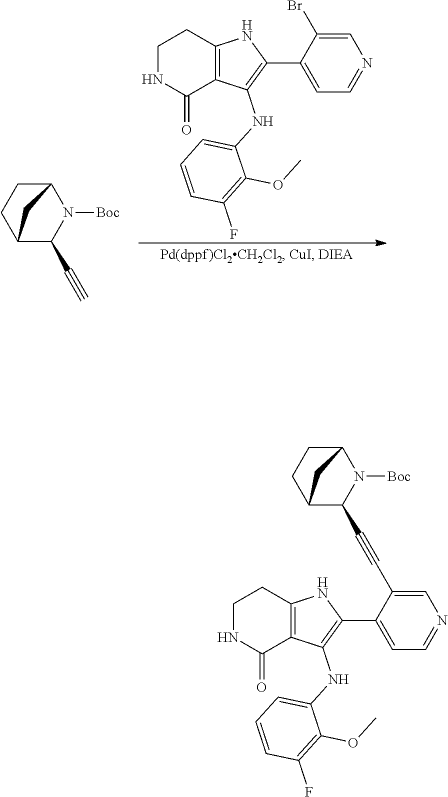 [SMILES: C#C[C@H]1[C@@H]2CC[C@@H](C2)N1C(=O)OC(C)(C)C ; COc1c(F)cccc1Nc1c(-c2ccncc2Br)[nH]c2c1C(=O)NCC2 ; COc1c(F)cccc1Nc1c(-c2ccncc2C#C[C@H]2[C@@H]3CC[C@@H](C3)N2C(=O)OC(C)(C)C)[nH]c2c1C(=O)NCC2]

To a stirred solution of tert-butyl (1S,3R,4R)-3-ethynyl-2-azabicyclo[2.2.1]heptane-2-carboxylate (154 mg, 0.696 mmol, 3 equiv) and 2-(3-bromopyridin-4-yl)-3-[(3-fluoro-2-methoxyphenyl)amino]-1H,5H,6H,7H-pyrrolo[3,2-c]pyridin-4-one (100 mg, 0.232 mmol, 1.00 equiv) and Pd(dppf)Cl₂·CH₂Cl₂ (47.2 mg, 0.058 mmol, 0.25 equiv) and CuI (22.1 mg, 0.116 mmol, 0.5 equiv) in DMF (1 mL) were added DIEA (149 mg, 1.16 mmol, 5 equiv) under argon atmosphere. The resulting suspension was backfilled with argon three times and stirred for overnight at 50° C. The resulting mixture was concentrated under vacuum. The residue was purified by reverse flash chromatography with the following conditions: (column, C18 silica gel; mobile phase, MeCN in water, 60% to 70% gradient in 10 min; detector, UV 254 nm). This resulted in tert-butyl (1S,3R,4R)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[2.2.1]heptane-2-carboxylate (80 mg, 60.35%) as a yellow solid.

LC-MS: (M+H)⁺ found 572.

### 71.2. Synthesis of 2-(3-{2-[(1S,3R,4R)-2-azabicyclo[2.2.1]heptan-3-yl]ethynyl}pyridin-4-yl)-3-[(3-fluoro-2-methoxyphenyl)amino]-1H,5H,6H,7H-pyrrolo[3,2-c]pyridin-4-one

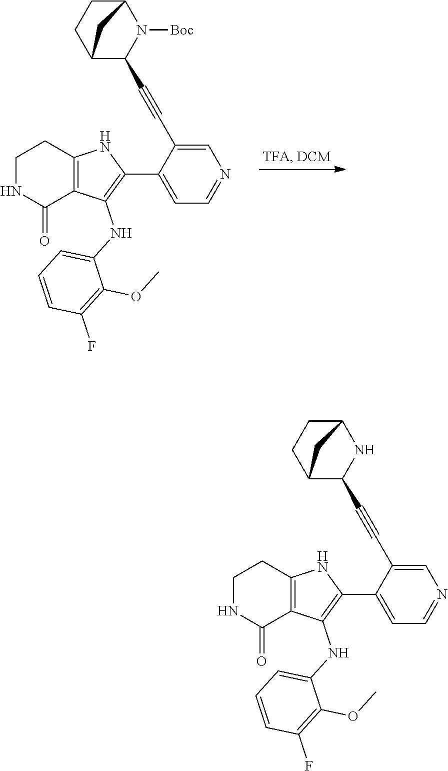 [SMILES: COc1c(F)cccc1Nc1c(-c2ccncc2C#C[C@H]2[C@@H]3CC[C@@H](C3)N2C(=O)OC(C)(C)C)[nH]c2c1C(=O)NCC2 ; COc1c(F)cccc1Nc1c(-c2ccncc2C#C[C@@H]2N[C@H]3CC[C@@H]2C3)[nH]c2c1C(=O)NCC2]

To a stirred solution of tert-butyl (1S,3R,4R)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[2.2.1]heptane-2-carboxylate (80 mg, 0.140 mmol, 1 equiv) in DCM (2 mL) was added TFA (0.7 mL) at room temperature under nitrogen atmosphere. The resulting mixture was stirred for 1 h and concentrated under reduced pressure to afford 2-(3-{2-[(1S,3R,4R)-2-azabicyclo[2.2.1]heptan-3-yl]ethynyl}pyridin-4-yl)-3-[(3-fluoro-2-methoxyphenyl)amino]-1H,5H,6H,7H-pyrrolo[3,2-c]pyridin-4-one (80 mg, crude) as a red oil.

LC-MS: (M+H)⁺ found 472.

### 71.3. Synthesis of 3-[(3-fluoro-2-methoxyphenyl)amino]-2-(3-{2-[(1S,3R,4R)-2-(prop-2-enoyl)-2-azabicyclo[2.2.1]heptan-3-yl]ethynyl}pyridin-4-yl)-1H,5H,6H,7H-pyrrolo[3,2-c]pyridin-4-one

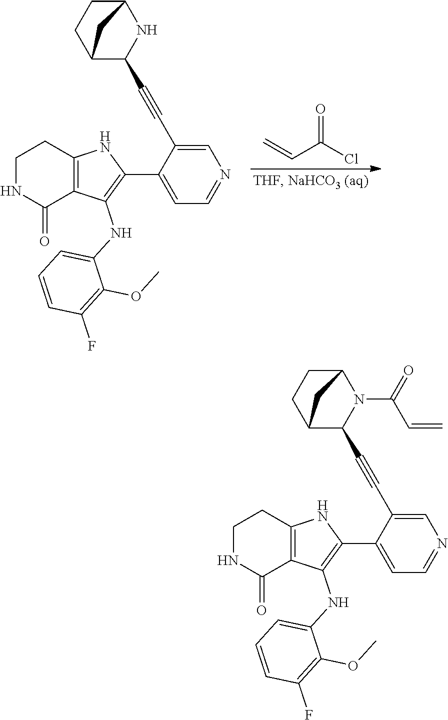

A mixture of 2-(3-{2-[(1S,3R,4R)-2-azabicyclo[2.2.1]heptan-3-yl]ethynyl}pyridin-4-yl)-3-[(3-fluoro-2-methoxyphenyl)amino]-1H,5H,6H,7H-pyrrolo[3,2-c]pyridin-4-one (80 mg, 0.170 mmol, 1 equiv) in THF (2 mL) was basified to pH 8 with NaHCO₃saturated solution. To the above mixture was added acryloyl chloride (13.82 mg, 0.153 mmol, 0.9 equiv) dropwise at 0° C. The resulting mixture was stirred for 2 h at room temperature. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (80 mg) that was purified by Prep-HPLC under the following conditions (Column:Xselect CSH C18 OBD Column 30*150 mm 5 μm, n Mobile Phase A:Water (0.1% FA), Mobile Phase B:ACN; Flow rate: 60 mL/min:Gradient: 24% B to 47% B in 8 min, 47% B; Wave Length; 254/220 nm; RT1(min); 8; Number Of Runs; 0). This resulted in 3-[(3-fluoro-2-methoxyphenyl)amino]-2-(3-{2-[(1S,3R,4R)-2-(prop-2-enoyl)-2-azabicyclo[2.2.1]heptan-3-yl]ethynyl}pyridin-4-yl)-1H,5H,6H,7H-pyrrolo[3,2-c]pyridin-4-one (42 mg, 46.6%) as a yellow solid.

LC-MS: (M+H)⁺ found 526.45.

¹H NMR (300 MHz, Chloroform-d) δ 11.00 (s, 1H), 8.53 (s, 1H), 8.09 (s, 1H), 7.88 (s, 1H), 7.47 (s, 1H), 6.63-6.28 (m, 5H), 6.07 (d, 1H), 5.81-5.69 (m, 1H), 5.28 (s, 1H), 4.47 (s, 1H), 4.42 (s, 1H), 4.10 (s, 3H), 3.68-3.55 (m, 2H), 3.353.24 (m, 2H), 2.87 (s, 1H), 2.32-2.21 (m, 1H), 1.97-1.50 (m, 4H).

### Example 72. N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxetan-3-yl}prop-2-enamide (compound 264)

### 72.1. Synthesis of N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxetan-3-yl}prop-2-enamide

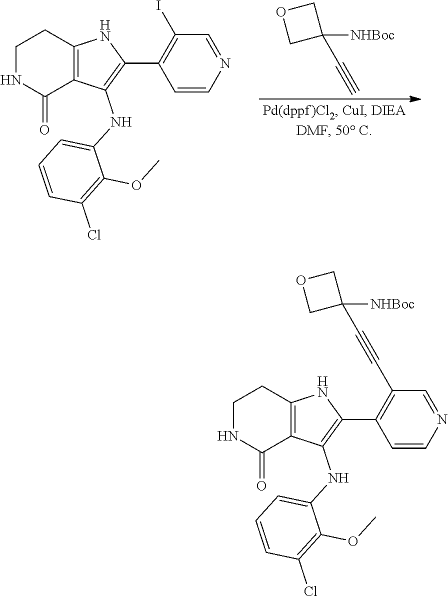 [SMILES: COc1c(Cl)cccc1Nc1c(-c2ccncc2I)[nH]c2c1C(=O)NCC2 ; C#CC1(NC(=O)OC(C)(C)C)COC1 ; COc1c(Cl)cccc1Nc1c(-c2ccncc2C#CC2(NC(=O)OC(C)(C)C)COC2)[nH]c2c1C(=O)NCC2]

To a stirred solution of 3-[(3-chloro-2-methoxyphenyl)amino]-2-(3-iodopyridin-4-yl)-1H,5H,6H,7H-pyrrolo[3,2-c]pyridin-4-one (120 mg, 0.24 mmol, 1.00 equiv) and Pd(dppf)Cl₂·CH₂Cl₂ (49 mg, 0.061 mmol, 0.25 equiv) and CuI (23 mg, 0.12 5 mmol, 0.50 equiv) in DMF (2 mL) were added tert-butyl N-(3-ethynyloxetan-3-yl)carbamate (239 mg, 1.21 mmol, 5.00 equiv) and DIEA (156 mg, 1.21 mmol, 5.00 equiv) under argon atmosphere. The resulting suspension was backfilled with argon three times and stirred for 2 h at 50° C. The resulting mixture was concentrated under vacuum. The residue was purified by reverse flash chromatography with the following conditions: (column, C18 silica gel; mobile phase, MeCN in water, 60% to 70% gradient in 10 min; detector, UV 254 nm) to afford tert-butyl (6R)-6-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-5-azaspiro[2.4]heptane-5-carboxylate (150 mg, 84.12%) as a yellow solid.

LC-MS: (M+H)⁺ found: 564.10.

### 72.2. Synthesis of 2-{3-[2-(3-aminooxetan-3-yl)ethynyl]pyridin-4-yl}-3-[(3-chloro-2-methoxyphenyl)amino]-1H,5H,6H,7H-pyrrolo[3,2-c]pyridin-4-one

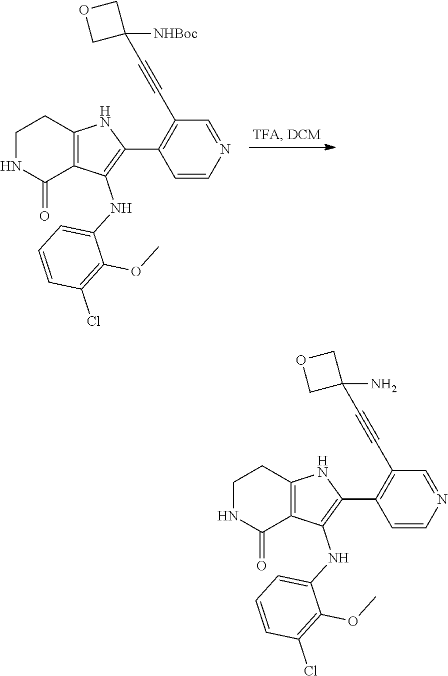 [SMILES: COc1c(Cl)cccc1Nc1c(-c2ccncc2C#CC2(NC(=O)OC(C)(C)C)COC2)[nH]c2c1C(=O)NCC2 ; COc1c(Cl)cccc1Nc1c(-c2ccncc2C#CC2(N)COC2)[nH]c2c1C(=O)NCC2]

To a stirred solution of tert-butyl N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxetan-3-yl}carbamate (100 mg, 0.17 mmol, 1.00 equiv) in DCM (3 mL) was added TFA (1 mL) at room temperature under nitrogen atmosphere. The resulting mixture was stirred for 1 h and concentrated under reduced pressure to afford 2-{3-[2-(3-aminooxetan-3-yl)ethynyl]pyridin-4-yl}-3-[(3-chloro-2-methoxyphenyl)amino]-1H,5H,6H,7H-pyrrolo[3,2-c]pyridin-4-one (100 mg, crude) as red oil.

LC-MS: (M+Na)⁺ found: 486.15.

### 72.3. Synthesis of N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxetan-3-yl}prop-2-enamide

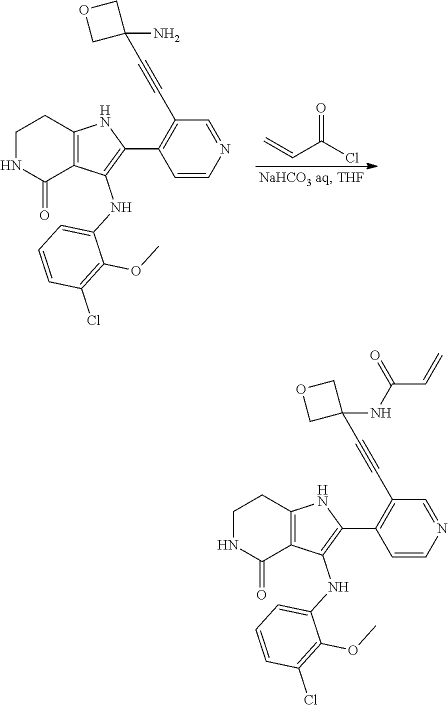

To a stirred solution of 2-{3-[2-(3-aminooxetan-3-yl)ethynyl]pyridin-4-yl}-3-[(3-chloro-2-methoxyphenyl)amino]-1H,5H,6H,7H-pyrrolo[3,2-c]pyridin-4-one (100 mg, 0.21 mmol, 1.00 equiv) in THF (1.5 mL) was basified to pH 8 with NaHCO₃(aq.). To the above mixture was added acryloyl chloride (23 mg, 0.25 mmol, 1.20 equiv) dropwise at 0° C. The resulting mixture was stirred for 1.5 h at room temperature under nitrogen atmosphere. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (130 mg) that was purified by Prep-HPLC under the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 23% B to 38% B in 10 min, 38% B; Wave Length: 254/220 nm; RT1(min): 10.38; Number Of Runs: 0) to afford N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxetan-3-yl}prop-2-enamide (9.9 mg, 8.87%) as a yellow solid.

LC-MS: (M+H)⁺ found: 518.10.

¹H NMR (300 MHz, DMSO-d6) δ 11.30 (s, 1H,), 9.45 (s, 1H), 8.59 (s, 1H), 8.32 (d, 1H), 7.50 (s, 1H), 7.35 (d, 1H), 7.16 (s, 1H), 6.72-6.63 (m, 2H), 6.36-6.17 (m, 2H), 6.14-6.12 (m, 1H), 5.76-5.72 (m, 1H), 4.90 (d, 2H,), 4.74 (d, 2H), 3.86 (s, 3H), 3.47-3.38 (m, 2H,), 2.93 (t, 2H).

### Example 73. N-{1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]cyclobutyl}prop-2-enamide (compound 262)

### 73.1. Synthesis of 2-{3-[2-(1-aminocyclobutyl) ethynyl] pyridin-4-yl}-3-[(3-fluoro-2-methoxyphenyl) amino]-1H,5H,6H,7H-pyrrolo[3,2-c] pyridin-4-one

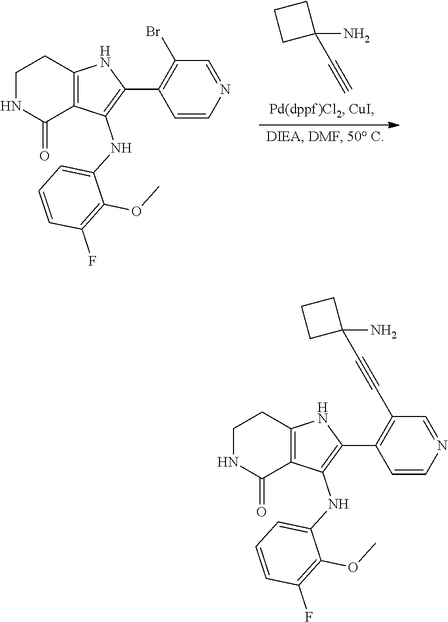

A mixture of 2-(3-bromopyridin-4-yl)-3-[(3-fluoro-2-methoxyphenyl) amino]-1H,5H,6H,7H-pyrrolo[3,2-c] pyridine -4-one (100 mg, 0.232 mmol, 1 equiv), 1-ethynylcyclobutan-1-amine (55 mg, 0.580 mmol, 2.5 equiv), Pd(dppf)Cl₂ (84.8 mg, 0.116 mmol, 0.5 equiv), CuI (22 mg, 0.116 mmol, 0.5 equiv) and DIEA (89.9 mg, 0.696 mmol, 3 equiv) in DMF (2 mL). The resulting mixture was stirred for 1 h at 50° C. under argon atmosphere. The resulting mixture was diluted with water and extracted with CH₂Cl₂, dried over anhydrous Na₂SO₄ and filtered. The filtrate was concentrated under reduced pressure to afford 2-{3-[2-(1-aminocyclobutyl) ethynyl] pyridin-4-yl}-3-[(3-fluoro-2-methoxyphenyl) amino]-1H,5H,6H,7H-pyrrolo[3,2-c] pyridin-4-one (100 mg, crude) as a light brown oil.

LC-MS: (M+H)⁺ found 446.0

### 73.2. Synthesis of N-{1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]cyclobutyl}prop-2-enamide

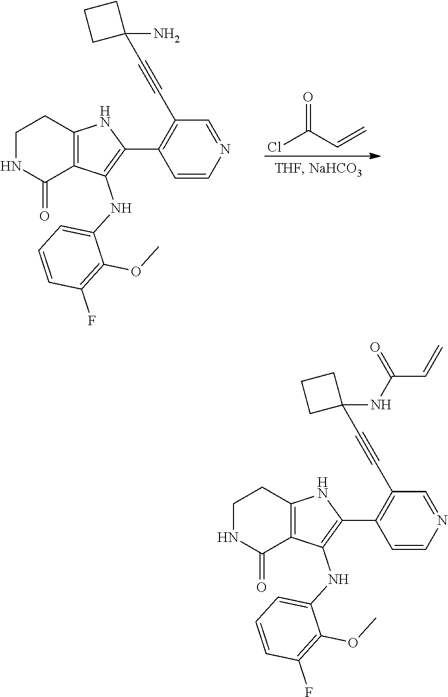

A solution of 2-{3-[2-(1-aminocyclobutyl) ethynyl] pyridin-4-yl}-3-[(3-fluoro-2-methoxyphenyl) amino]-1H,5H,6H,7H-pyrrolo[3,2-c] pyridin-4-one (100 mg, 0.224 mmol, 1 equiv) in THF (2 mL) was basified to pH 8 with sat. NaHCO₃(aq.). To the above mixture was added acryloyl chloride (23.3 mg, 0.258 mmol, 1.15 equiv) at 0° C. The resulting mixture was stirred for 30 min at room temperature under nitrogen atmosphere. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (50 mg) that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 28% B to 48% B in 10 min, 48% B; Wave Length: 254/220 nm) to afford N-{1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]cyclobutyl}prop-2-enamide (8.4 mg, 7.23%) as a light brown solid.

LC-MS: (M+H)⁺ found 500.15

¹H NMR (400 MHz, DMSO-d6) δ 11.44 (s, 1H), 9.11 (s, 1H), 8.55 (s, 1H), 8.29-8.21 (m, 1H), 7.59 (s, 1H), 7.37 (d, 1H), 7.20 (s, 1H), 6.69-6.61 (m, 1H), 6.52 (t, 1H), 6.35-6.17 (m, 2H), 6.02 (d, 1H), 5.74-5.65 (m, 1H), 3.92 (s, 3H), 3.48-3.39 (m, 2H), 2.98 (t, 2H), 2.62-2.53 (m, 2H), 2.45-2.38 (m, 2H), 2.11-1.96 (m, 2H).

Example 74. N-{1-[2-(4-{3-[(3-chloro-2-methoxyphenyl) amino]-4-oxo-1H,5H,6H,7H-pyrrolo[3,2-c] pyridin-2-yl} pyridin-3-yl) ethynyl] cyclobutyl} prop-2-enamide (compound 261)

### 74.1. Synthesis of 2-{3-[2-(1-aminocyclobutyl) ethynyl] pyridin-4-yl}-3-[(3-chloro-2-methoxyphenyl) amino]-1H,5H,6H,7H-pyrrolo[3,2-c] pyridin-4-one

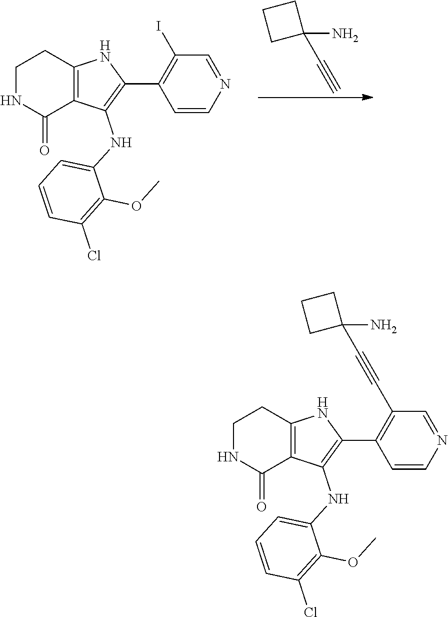

To a stirred solution of 3-[(3-chloro-2-methoxyphenyl) amino]-2-(3-iodopyridin-4-yl)-1H,5H,6H,7H-pyrrolo[3,2-c] pyridin-4-one (200 mg, 0.404 mmol, 1 equiv) and 1-ethynylcyclobutan-1-amine (96.2 mg, 1.010 mmol, 2.5 equiv) in DMF (2 mL) were added CuI (38.50 mg, 0.202 mmol, 0.5 equiv) and Pd(dppf)Cl2CH2Cl2 (165 mg, 0.202 mmol, 0.5 equiv) and DIEA (156 mg, 1.21 mmol, 3 equiv) at room temperature under argon atmosphere. The resulting suspension was backfilled with argon three times and stirred for 1 h at 50° C. The resulting mixture was diluted with water and extracted with CH₂Cl₂. The organic layer was dried over anhydrous Na₂SO₄. After filtration, the filtrate was concentrated under reduced pressure to afford 2-{3-[2-(1-aminocyclobutyl) ethynyl] pyridin-4-yl}-3-[(3-chloro-2-methoxyphenyl) amino]-1H,5H,6H,7H-pyrrolo[3,2-c] pyridin-4-one (100 mg, crude) as a yellow oil.

LC-MS: M+H found: 462.0.

### 74.2. Synthesis of N-{1-[2-(4-{3-[(3-chloro-2-methoxyphenyl) amino]-4-oxo-1H,5H,6H,7H-pyrrolo[3,2-c] pyridin-2-yl} pyridin-3-yl) ethynyl] cyclobutyl} prop-2-enamide

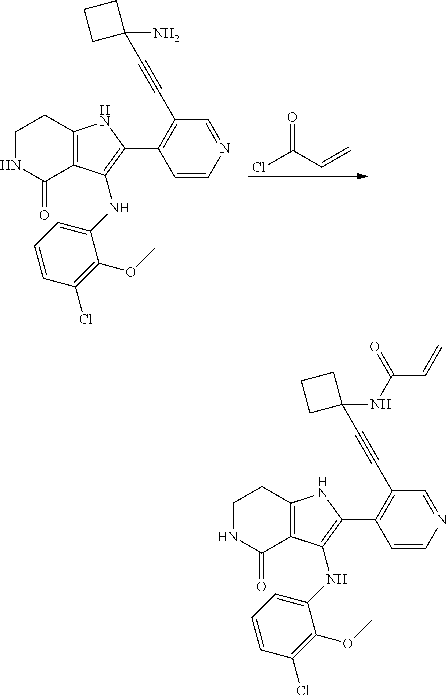

To a solution of 2-{3-[2-(1-aminocyclobutyl) ethynyl]pyridin-4-yl}-3-[(3-chloro-2-methoxyphenyl)amino]-1H,5H,6H,7H-pyrrolo[3,2-c]pyridin-4-one (100 mg, 0.216 mmol, 1 equiv) in THF (2 mL) was basified to pH 8 with sat. NaHCO₃(aq.). To the above mixture was added was added acryloyl chloride (22.5 mg, 0.248 mmol, 1.15 10 equiv) at 0° C. under nitrogen atmosphere. The resulting mixture was stirred for 1 h at room temperature under nitrogen atmosphere. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (60 mg) that was purified by Prep-HPLC under the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 28% B to 58% B in min, 58% B; Wave Length: 220/254 nm; RT1(min): 10.37; Number Of Runs: 0) to afford N-{1-[2-(4-{3-[(3-chloro-2-methoxyphenyl) amino]-4-oxo-1H,5H,6H,7H-pyrrolo[3,2-c] pyridin-2-yl} pyridin-3-yl) ethynyl] cyclobutyl} prop-2-enamide (9.4 mg, 8.36%) as a white solid.

LC-MS: M+H found: 516.10.

¹H NMR (400 MHz, DMSO-d6) δ 11.46 (s, 1H), 9.10 (s, 1H), 8.55 (s, 1H), 8.27 (d, 1H), 7.57 (s, 1H), 7.36 (d, 1H), 7.19 (d, 1H), 6.82-6.59 (m, 2H), 6.36-6.07 (m, 3H), 5.79-5.60 (m, 1H), 3.88 (s, 3H), 3.48-3.42 (m, 2H), 2.97 (t, 2H), 2.63-2.54 (m, 2H), 2.46-2.35 (m, 2H), 2.04-1.86 (m, 2H).

### Example 75. 2-(3-{[(3R)-4-(but-2-ynoyl)morpholin-3-yl]methoxy}pyridin-4-yl)-3-[(3-fluoro-2-methoxyphenyl)amino]-1H,5H,6H,7H-pyrrolo[3,2-c]pyridin-4-one (compound 293)

### 75.1. Synthesis of 3-[(3-fluoro-2-methoxyphenyl) amino]-2-{3-[(3R)-morpholin-3-ylmethoxy] pyridin-4-yl}-1H,5H,6H,7H-pyrrolo[3,2-c] pyridin-4-one

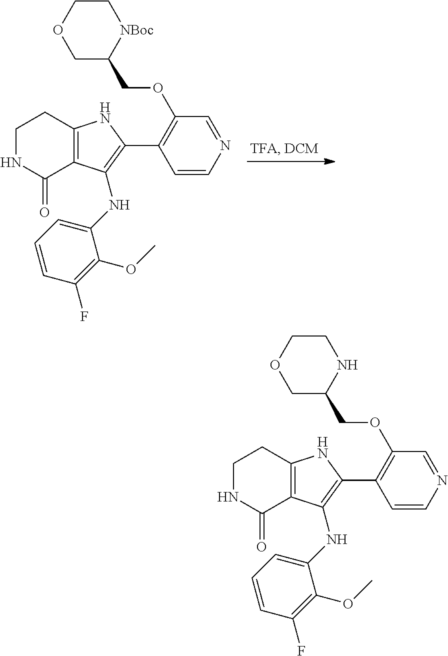 [SMILES: COc1c(F)cccc1Nc1c(-c2ccncc2OC[C@H]2COCCN2C(=O)OC(C)(C)C)[nH]c2c1C(=O)NCC2 ; COc1c(F)cccc1Nc1c(-c2ccncc2OC[C@H]2COCCN2)[nH]c2c1C(=O)NCC2]

To a stirred solution of tert-butyl (3R)-3-[({4-[5-(tert-butoxycarbonyl)-4-oxo-1H,6H,7H-pyrrolo[3,2-c] pyridin-2-yl] pyridin-3-yl} oxy) methyl] morpholine-4-carboxylate (75 mg, 0.142 mmol, 1 equiv) in DCM (1 mL) was added TFA (0.3 mL) at room temperature under nitrogen atmosphere. The resulting mixture was stirred for 1 h and concentrated under reduced pressure to afford 3-[(3-fluoro-2-methoxyphenyl) amino]-2-{3-[(3R)-morpholin-3-ylmethoxy] pyridin-4-yl}-1H,5H,6H,7H-pyrrolo[3,2-c] pyridin-4-one (90 mg, crude) as a red oil.

LC-MS: M+H found: 468.0.

### 75.2. Synthesis of 2-(3-{[(3R)-4-(but-2-ynoyl)morpholin-3-yl]methoxy}pyridin-4-yl)-3-[(3-fluoro-2-methoxyphenyl)amino]-1H,5H,6H,7H-pyrrolo[3,2-c]pyridin-4-one

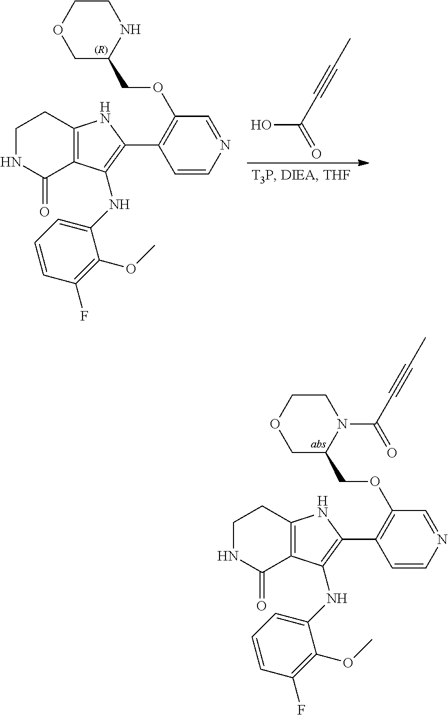

To a stirred solution of 3-[(3-fluoro-2-methoxyphenyl) amino]-2-{3-[(3R)-morpholin-3-ylmethoxy] pyridin-4-yl}-1H,5H,6H,7H-pyrrolo[3,2-c] pyridin-4-one (75 mg, 0.160 mmol, 1 equiv) in THF (1 mL) was basified to pH 8 with DIEA. To the above mixture was added 2-butynoic acid (20.2 mg, 0.240 mmol, 1.5 equiv) at 0° C. under nitrogen atmosphere followed by the addition of T3P (102 mg, 0.320 mmol, 2.0 equiv, 50% in EA) dropwise. The resulting mixture was stirred for 1 h at room temperature under nitrogen atmosphere. Saturated aqueous NaHCO₃ (10 mL) was added to the reaction mixture at 0° C. and extracted with EtOAc (3×10 mL), dried over anhydrous Na₂SO₄ and filtered. The filtrate was concentrated under reduced pressure to give crude product (100 mg) that was purified by Prep-HPLC under the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 23% B to 39% B in 8 min, 39% B; Wave Length: 254/220 nm; RT1(min): 8; Number Of Runs: 0) to afford 2-(3-{[(3R)-4-(but-2-ynoyl)morpholin-3-yl]methoxy}pyridin-4-yl)-3-[(3-fluoro-2-methoxyphenyl)amino]-1H,5H,6H,7H-pyrrolo[3,2-c]pyridin-4-one (27.8 mg, 32.19%) as a yellow solid.

LC-MS: M+H found: 534.10.

¹H NMR (400 MHz, Chloroform-d) δ 10.30 (s, 1H), 8.32 (s, 1H), 8.01 (d, 1H), 7.72-7.42 (m, 2H), 6.62-6.58 (m, 1H), 6.52-6.47 (m, 1H), 6.06-6.02 (m, 1H), 5.28-5.01 (m, 2H), 4.80 (t, 1H), 4.35-4.18 (m, 2H), 4.16-3.93 (m, 5H), 3.76-3.72 (m, 1H), 3.70-3.48 (m, 4H), 3.39-3.03 (m, 2H), 2.03 (s, 3H).

### Example 76. 3-[(3-fluoro-2-methylphenyl)amino]-2-(3-{2-[(2R)-2-methyl-1-(prop-2-enoyl)pyrrolidin-2-yl]ethynyl}pyridin-4-yl)-1H,5H,6H,7H-pyrrolo[3,2-c]pyridin-4-one (compound 304)

### 76.1. Synthesis of 1-fluoro-3-isothiocyanato-2-methylbenzene

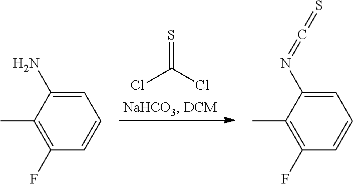

To a stirred solution of 3-fluoro-2-methyl-aniline (10 g, 79.9 mmol, 1.00 equiv) in NaHCO₃ (60 mL) and DCM (60 mL) was added thiophosgene (9.2 g, 79.9 mmol, 1.00 equiv) dropwise at 0° C. The resulting mixture was stirred for 2 h at room temperature. The resulting mixture was extracted with DCM (3×60 mL). The combined organic layers were washed with brine (180 mL), dried over anhydrous Na₂SO₄. After filtration, the filtrate was concentrated under reduced pressure. The residue was purified by silica gel column chromatography, eluted with PE to afford 1-fluoro-3-isothiocyanato-2-methylbenzene (11.3 g, 84.58%) as a yellow oil.

LC-MS: (M+H)⁺ found 168.00.

### 76.2. Synthesis of N-(3-fluoro-2-methylphenyl)-4-hydroxy-2-oxo-5,6-dihydro-1H-pyridine-3-carbothioamide

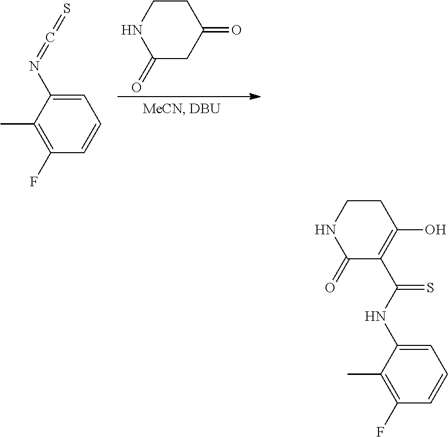 [SMILES: Cc1c(F)cccc1N=C=S ; O=C1CCNC(=O)C1 ; Cc1c(F)cccc1NC(=S)C1=C(O)CCNC1=O]

To a stirred solution of 1-fluoro-3-isothiocyanato-2-methylbenzene (11.3 g, 67.5 mmol, 1.00 equiv) and piperidine-2,4-dione (7.6 g, 67.5 mmol, 1.00 equiv) in ACN (100 mL) was added DBU (15.1 mL, 101 mmol, 1.50 equiv) dropwise at 0° C. The resulting mixture was stirred for overnight at room temperature. Desired product could be detected by LCMS. The mixture was acidified to pH 3 with 1 N HCl. The precipitated solids were collected by filtration and washed with MeCN (3×15 mL) The resulting solid was dried under vacuum to afford N-(3-fluoro-2-methylphenyl)-4-hydroxy-2-oxo-5,6-dihydro-1H-pyridine-3-carbothioamide (17.1 g, crude) as a yellow solid.

LC-MS: (M+H)⁺ found 280.95.

### 76.3. Synthesis of 4-{[(3-bromopyridin-4-yl) methyl] amino}-N-(3-fluoro-2-methylphenyl)-2-oxo-5,6-dihydro-1H-pyridine-3-carbothioamide

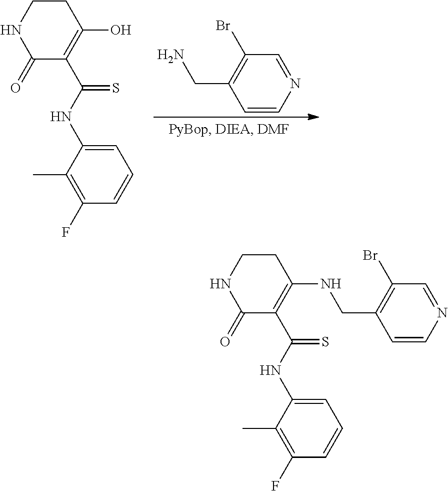 [SMILES: Cc1c(F)cccc1NC(=S)C1=C(O)CCNC1=O ; NCc1ccncc1Br ; Cc1c(F)cccc1NC(=S)C1=C(NCc2ccncc2Br)CCNC1=O]

To a stirred solution of N-(3-fluoro-2-methylphenyl)-4-hydroxy-2-oxo-5,6-dihydro-1H-pyridine-3-carbothioamide (17.1 g, 61.0 mmol, 1.00 equiv) and 1-(3-bromopyridin-4-yl) methanamine (12.5 g, 67.1 mmol, 1.10 equiv) in DMF (171 mL) were added DIEA (23.6 g, 183 mmol, 3.00 equiv) and PyBOP (47.6 g, 91.5 mmol, 1.50 equiv) in portions at 0° C. The resulting mixture was stirred for overnight at room temperature. Desired product could be detected by LCMS. The resulting mixture was diluted with water (400 mL). The precipitated solids were collected by filtration and washed with water (3×20 mL) and MeOH (3×20 mL). The resulting solid was dried under vacuum to afford 4-{[(3-bromopyridin-4-yl) methyl] amino}-N-(3-fluoro-2-methylphenyl)-2-oxo-5,6-dihydro-1H-pyridine-3-carbothioamide (15 g, crude) as an off-white solid.

LC-MS: (M+H)⁺ found 448.85.

### 76.4. Synthesis of 2-(3-bromopyridin-4-yl)-3-[(3-fluoro-2-methylphenyl) amino]-1H,5H,6H,7H-pyrrolo[3,2-c] pyridin-4-one

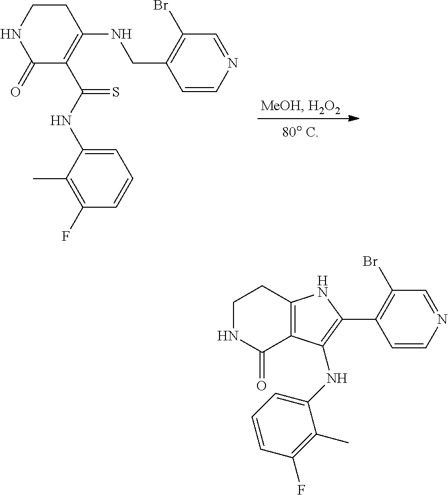 [SMILES: Cc1c(F)cccc1NC(=S)C1=C(NCc2ccncc2Br)CCNC1=O ; Cc1c(F)cccc1Nc1c(-c2ccncc2Br)[nH]c2c1C(=O)NCC2]

A mixture of 4-{[(3-bromopyridin-4-yl) methyl] amino}-N-(3-fluoro-2-methylphenyl)-2-oxo-5,6-dihydro-1H-pyridine-3-carbothioamide (15.0 g, 33.3 mmol, 1.00 equiv) and H₂O₂ (300%) (2.59 mL, 33.3 mmol, 1.00 equiv, 30%) in MeOH (150 mL). The resulting mixture was stirred for overnight at 80° C. under nitrogen atmosphere. Desired product could be detected by LCMS. The resulting mixture was concentrated under reduced pressure. The residue was purified by reverse flash chromatography with the following conditions: column, C18; mobile phase, MeCN in water, 40% to 45% gradient in 10 min; detector, UV 254 nm. to afford 2-(3-bromopyridin-4-yl)-3-[(3-fluoro-2-methylphenyl) amino]-1H,5H,6H,7H-pyrrolo[3,2-c] pyridin-4-one (2.5 g, 18.03%) as a yellow solid.

LC-MS: (M+H)⁺ found 414.85.

### 76.5. Synthesis of tert-butyl (2R)-2-formyl-2-methylpyrrolidine-1-carboxylate

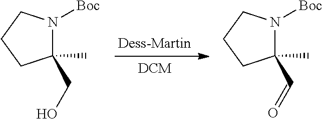

To a stirred solution of tert-butyl (2R)-2-(hydroxymethyl)-2-methylpyrrolidine-1-carboxylate (0.74 g, 3.43 mmol, 1.00 equiv) in DCM (10 mL) was added Dess-Martin (2.19 g, 5.16 mmol, 1.50 equiv) in portions at 0° C. The resulting mixture was stirred for 2 h at room temperature. The reaction was quenched by the addition of sat. Na₂SO₃ (aq.) (10 mL) at 0° C. The mixture was extracted with EtOAc (3×20 mL). The combined organic layers were washed with brine (60 mL), dried over anhydrous Na₂SO₄ and filtered. The filtrate was concentrated under reduced pressure and purified by silica gel column chromatography, eluted with PE/EA (8:1) to afford tert-butyl (2R)-2-formyl-2-methylpyrrolidine-1-carboxylate (640 mg, 87.30%) as a colorless oil. 76.6. Synthesis of tert-butyl (2R)-2-ethynyl-2-methylpyrrolidine-1-carboxylate

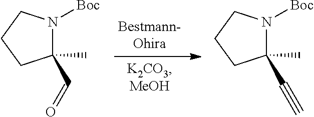

To a stirred mixture of tert-butyl (2R)-2-formyl-2-methylpyrrolidine-1-carboxylate (640 mg, 3.00 mmol, 1.00 equiv) and K₂CO₃ (829 mg, 6.00 mmol, 2.00 equiv) in MeOH (10 mL) was added Bestmann-Ohira reagent (691 mg, 3.60 mmol, 1.20 equiv) dropwise at 0° C. The resulting mixture was stirred for 2 h at room temperature. The reaction was quenched with water at 0° C. and extracted with EtOAc (3×10 mL), dried over anhydrous Na₂SO₄ and filtered. The filtrate was concentrated under reduced pressure and purified by silica gel column chromatography, eluted with PE/EA (8:1) to afford tert-butyl (2R)-2-ethynyl-2-methylpyrrolidine-1-carboxylate (450 mg, 71.65%) as a colorless oil.

### 76.7. Synthesis of tert-butyl (2R)-2-[2-(4-{3-[(3-fluoro-2-methylphenyl) amino]-4-oxo-1H,5H,6H,7H-pyrrolo[3,2-c] pyridin-2-yl} pyridin-3-yl) ethynyl]-2-methylpyrrolidine-1-carboxylate

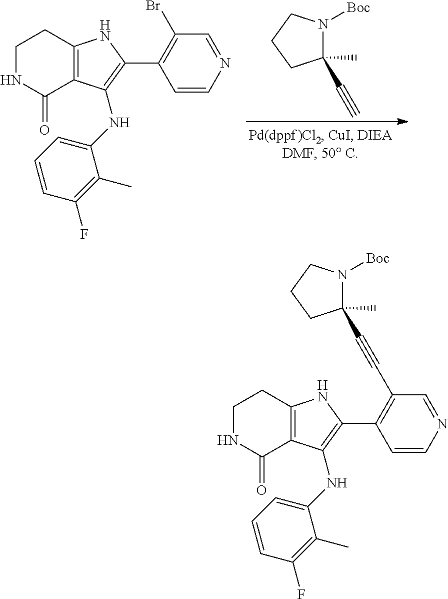 [SMILES: Cc1c(F)cccc1Nc1c(-c2ccncc2Br)[nH]c2c1C(=O)NCC2 ; C#C[C@@]1(C)CCCN1C(=O)OC(C)(C)C ; Cc1c(F)cccc1Nc1c(-c2ccncc2C#C[C@@]2(C)CCCN2C(=O)OC(C)(C)C)[nH]c2c1C(=O)NCC2]

To a stirred mixture of tert-butyl (2R)-2-ethynyl-2-methylpyrrolidine-1-carboxylate (226 mg, 1.08 mmol, 3.00 equiv), CuI (6 mg, 0.036 mmol, 0.10 equiv), DIEA (186 mg, 1.44 mmol, 4.00 equiv) and 2-(3-bromopyridin-4-yl)-3-[(3-fluoro-2-methylphenyl) amino]-1H,5H,6H,7H-pyrrolo[3,2-c] pyridin-4-one (150 mg, 0.36 mmol, 1.00 equiv) in DMF (2.5 mL) were added Pd(dppf)Cl₂·CH₂Cl₂ (29 mg, 0.036 mmol, 0.10 equiv) under argon atmosphere. The resulting mixture was stirred for overnight at 50° C. under argon atmosphere. LCMS confirmed completion of reaction and desired product was observed. The resulting mixture was diluted with water (12.5 mL). The precipitated solids were collected by filtration and washed with water (3×5 mL). The crude product was purified by trituration with Et₂O (10 mL) to afford tert-butyl (2R)-2-[2-(4-{3-[(3-fluoro-2-methylphenyl) amino]-4-oxo-1H,5H,6H,7H-pyrrolo[3,2-c] pyridin-2-yl} pyridin-3-yl) ethynyl]-2-methylpyrrolidine-1-carboxylate (80 mg, crude) as a yellow solid.

LC-MS: (M+H)⁺ found 544.15.

### 76.8. Synthesis of 3-[(3-fluoro-2-methylphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one

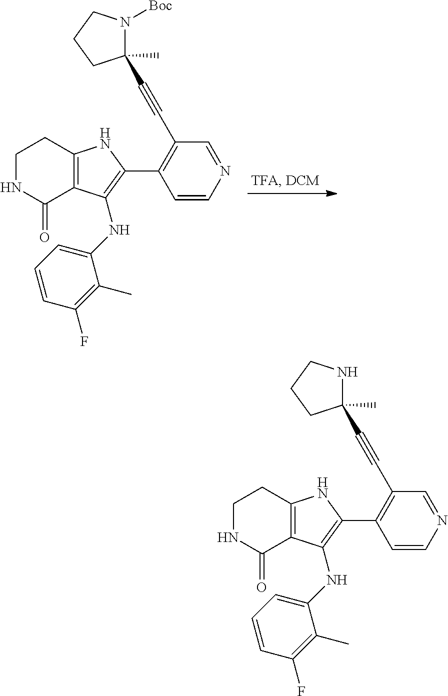 [SMILES: Cc1c(F)cccc1Nc1c(-c2ccncc2C#C[C@@]2(C)CCCN2C(=O)OC(C)(C)C)[nH]c2c1C(=O)NCC2 ; Cc1c(F)cccc1Nc1c(-c2ccncc2C#C[C@@]2(C)CCCN2)[nH]c2c1C(=O)NCC2]

To a stirred mixture of tert-butyl (2R)-2-[2-(4-{3-[(3-fluoro-2-methylphenyl) amino]-4-oxo-1H,5H,6H,7H-pyrrolo[3,2-c] pyridin-2-yl} pyridin-3-yl) ethynyl]-2-methylpyrrolidine-1-carboxylate (80 mg, 0.15 mmol, 1.00 equiv) in DCM (1 mL) was add TFA (0.3 mL) at room temperature under nitrogen atmosphere. The resulting mixture was stirred for 1 h and concentrated under reduced pressure to afford 3-[(3-fluoro-2-methylphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (160 mg, crude) as a red oil.

LC-MS: (M+H)⁺ found 444.10.

### 76.9. Synthesis of 3-[(3-fluoro-2-methylphenyl)amino]-2-(3-{2-[(2R)-2-methyl-1-(prop-2-enoyl)pyrrolidin-2-yl]ethynyl}pyridin-4-yl)-1H,5H,6H,7H-pyrrolo[3,2-c]pyridin-4-one

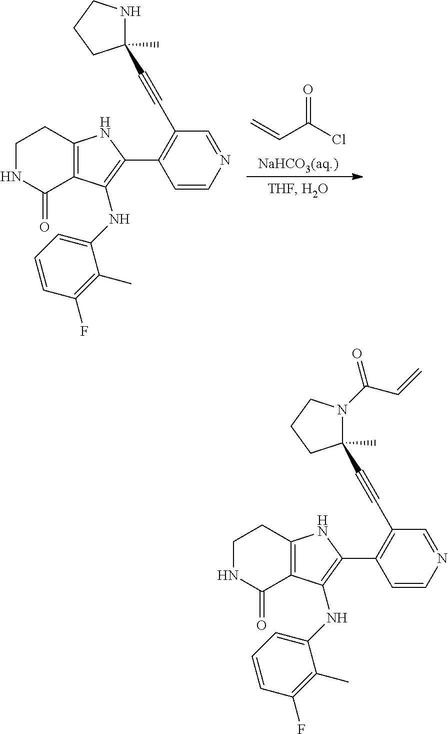

To a stirred mixture of 3-[(3-fluoro-2-methylphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (80 mg, 0.180 mmol, 1.00 equiv) in THF (1 mL) was basified to pH 8 with NaHCO₃(aq.). To the above mixture was added acryloyl chloride (16 mg, 0.18 mmol, 1.00 equiv) dropwise at 0° C. under nitrogen atmosphere. The resulting mixture was stirred for 30 min at 0° C. under nitrogen atmosphere. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (50 mg) that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 38% B to 52% B in 10 min, 52% B; Wave Length: 254/220 nm; RT1(min): 8.85; Number Of Runs: 0) to afford 3-[(3-fluoro-2-methylphenyl)amino]-2-(3-{2-[(2R)-2-methyl-1-(prop-2-enoyl)pyrrolidin-2-yl]ethynyl}pyridin-4-yl)-1H,5H,6H,7H-pyrrolo[3,2-c]pyridin-4-one (5.3 mg, 5.87%) as a yellow solid.

LC-MS: (M+H)⁺ found 498.20.

¹H NMR (300 MHz, Chloroform-d) δ 11.39 (s, 1H), 8.67-7.80 (m, 2H), 7.44 (s, 1H), 7.20-7.10 (m, 1H), 6.71-6.59 (m, 1H), 6.47-6.25 (m, 3H), 6.16 (d, 1H), 5.71-5.63 (m, 1H), 5.15 (s, 1H), 3.78-3.61 (m, 2H), 3.59-3.45 (m, 2H), 3.27-3.10 (m, 2H), 2.54-2.37 (m, 1H), 2.26 (s, 3H), 2.17-1.94 (m, 3H), 1.71 (s, 3H).

### Example 77. 2-(3-{2-[(2R)-1-[(2E)-4-(dimethylamino)but-2-enoyl]pyrrolidin-2-yl]ethynyl}pyridin-4-yl)-3-[(3-fluoro-2-methoxyphenyl)amino]-1H,5H,6H,7H-pyrrolo[3,2-c]pyridin-4-one (compound 303)

### 77.1. Synthesis of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]pyrrolidine-1-carboxylate

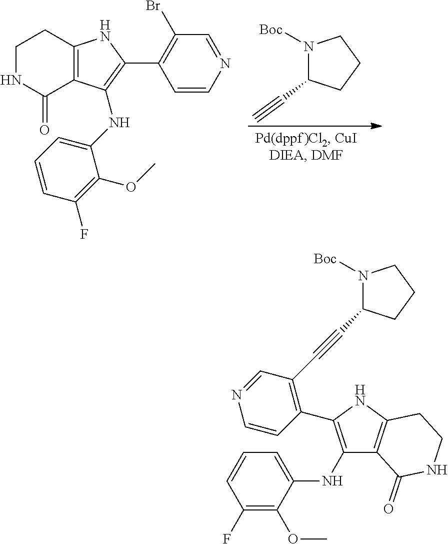 [SMILES: COc1c(F)cccc1Nc1c(-c2ccncc2Br)[nH]c2c1C(=O)NCC2 ; C#C[C@H]1CCCN1C(=O)OC(C)(C)C ; COc1c(F)cccc1Nc1c(-c2ccncc2C#C[C@H]2CCCN2C(=O)OC(C)(C)C)[nH]c2c1C(=O)NCC2]

A mixture of 2-(3-bromopyridin-4-yl)-3-[(3-fluoro-2-methoxyphenyl)amino]-1H,5H,6H,7H-pyrrolo[3,2-c]pyridin-4-one (150 mg, 0.348 mmol, 1 equiv), tert-butyl (2R)-2-ethynylpyrrolidine-1-carboxylate (135.83 mg, 0.696 mmol, 2 equiv), Pd(dppf)Cl₂CH₂Cl₂ (70.8 mg, 0.087 mmol, 0.25 equiv), CuI (33.1 mg, 0.174 mmol, 0.5 equiv) and DIEA (225 mg, 1.74 mmol, 5 equiv) in DMF (5 mL) at room temperature under argon atmosphere. The resulting mixture was stirred overnight at 50° C. under argon atmosphere. The resulting mixture was concentrated under vacuum. The residue was purified by reverse flash chromatography with the following conditions: column, C18 silica gel; mobile phase, MeCN in water, 10% to 70% gradient in 40 min; detector, UV 254 nm. This resulted in tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]pyrrolidine-1-carboxylate (120 mg, 63.23%) as a yellow oil.

LC-MS: (M+H)⁺ found 546.30.

### 77.2. Synthesis of 3-[(3-fluoro-2-methoxyphenyl)amino]-2-(3-{2-[(2R)-pyrrolidin-2-yl]ethynyl}pyridin-4-yl)-1H,5H,6H,7H-pyrrolo[3,2-c]pyridin-4-one

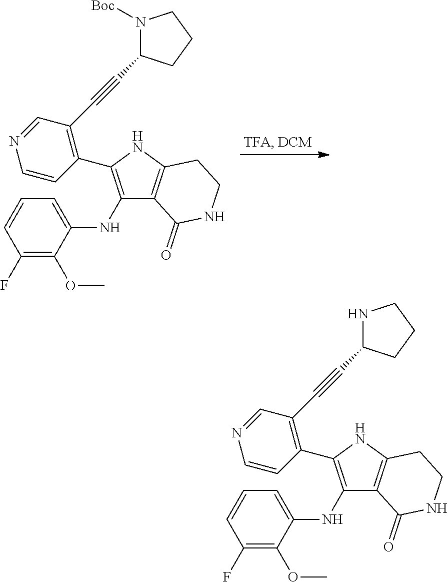 [SMILES: COc1c(F)cccc1Nc1c(-c2ccncc2C#C[C@H]2CCCN2C(=O)OC(C)(C)C)[nH]c2c1C(=O)NCC2 ; COc1c(F)cccc1Nc1c(-c2ccncc2C#C[C@H]2CCCN2)[nH]c2c1C(=O)NCC2]

To a stirred solution of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]pyrrolidine-1-carboxylate (100 mg, 0.183 mmol, 1 equiv) in DCM (3 mL) was added TFA (1 mL) at room temperature under nitrogen atmosphere. The resulting mixture was stirred for 1 h and concentrated under reduced pressure to afford 3-[(3-fluoro-2-methoxyphenyl)amino]-2-(3-{2-[(2R)-pyrrolidin-2-yl]ethynyl}pyridin-4-yl)-1H,5H,6H,7H-pyrrolo[3,2-c]pyridin-4-one (120 mg, crude) as a yellow oil.

LC-MS: (M+H)⁺ found 446.05.

### 77.3. Synthesis of 2-(3-{2-[(2R)-1-[(2E)-4-(dimethylamino)but-2-enoyl]pyrrolidin-2-yl]ethynyl}pyridin-4-yl)-3-[(3-fluoro-2-methoxyphenyl)amino]-1H,5H,6H,7H-pyrrolo[3,2-c]pyridin-4-one

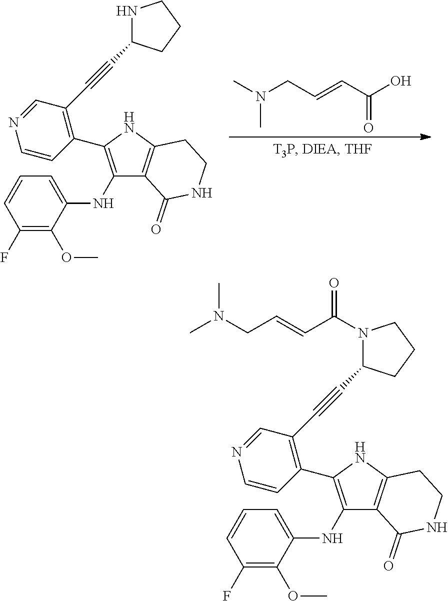

To a stirred solution of 3-[(3-fluoro-2-methoxyphenyl)amino]-2-(3-{2-[(2R)-pyrrolidin-2-yl]ethynyl}pyridin-4-yl)-1H,5H,6H,7H-pyrrolo[3,2-c]pyridin-4-one (110 mg, 0.247 mmol, 1 equiv) in THF (2 mL) was basified to pH 8 with DIEA. To the above mixture was added (2E)-4-(dimethylamino)but-2-enoic acid (47.9 mg, 0.371 mmol, 1.5 equiv) at 0° C. under nitrogen atmosphere followed by the addition of T3P (157 mg, 0.494 mmol, 2.0 equiv, 50% in EA) dropwise. The resulting mixture was stirred for 1 h at room temperature under nitrogen atmosphere. Saturated aqueous NaHCO₃ (10 mL) was added to the reaction mixture at 0° C. and extracted with EtOAc (3×10 mL), dried over anhydrous Na₂SO₄ and filtered. The filtrate was concentrated under reduced pressure to give crude product (100 mg) that was purified by Prep-HPLC under the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 24% B in 8 min, 24% B; Wave Length: 254/220 nm; RT1(min): 8;) to afford 2-(3-{2-[(2R)-1-[(2E)-4-(dimethylamino)but-2-enoyl]pyrrolidin-2-yl]ethynyl}pyridin-4-yl)-3-[(3-fluoro-2-methoxyphenyl)amino]-1H,5H,6H,7H-pyrrolo[3,2-c]pyridin-4-one (44 mg, 31.9%) as a yellow solid.

LC-MS: (M+H)⁺ found 557.6.

¹H NMR (400 MHz, Chloroform-d) δ 11.05 (s, 1H), 8.53 (s, 1H), 8.15 (s, 1H), 7.70 (s, 1H), 7.47 (s, 1H), 6.94-6.88 (m, 1H), 6.59-6.53 (m, 2H), 6.48-6.43 (m, 1H), 6.09-6.07 (m, 1H), 5.30 (s, 1H), 4.89 (t, 1H), 4.09 (s, 3H), 3.85-3.75 (m, 1H), 3.71-3.64 (m, 1H), 3.56-3.64 (m, 2H), 3.54-3.49 (m, 2H), 3.23 (t, 2H), 2.60 (s, 6H), 2.35-2.31 (m, 1H), 2.29-2.12 (m, 2H), 2.10-2.09 (m, 1H).

### Example 78. 3-[(3-fluoro-2-methoxyphenyl)amino]-2-{3-[(1S)-1-[(2R)-1-(prop-2-enoyl)azetidin-2-yl]ethoxy]pyridin-4-yl}-1H,5H,6H,7H-pyrrolo[3,2-c]pyridin-4-one (compound 310)

### 78.1. Synthesis of tert-butyl (2S)-2-[methoxy(methyl)carbamoyl]azetidine-1-carboxylate

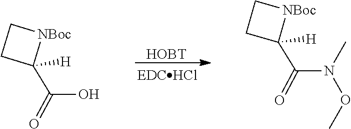 [SMILES: CC(C)(C)OC(=O)N1CC[C@@H]1C(=O)O ; "HOBT" ; CON(C)C(=O)[C@H]1CCN1C(=O)OC(C)(C)C]

To a stirred solution of (2S)-1-(tert-butoxycarbonyl)azetidine-2-carboxylic acid (10 g, 49.7 mmol, 1 equiv) and methoxy(methyl)amine hydrochloride (5.82 g, 59.6 mmol, 1.2 equiv) in DMF (200 mL) were added N-Methylmorpholine (6.03 g, 59.6 mmol, 1.2 equiv), HOBT (8.06 g, 59.6 mmol, 1.2 equiv) and EDCI (11.4 g, 59.6 mmol, 1.2 equiv) at 0° C. under nitrogen atmosphere. The resulting mixture was stirred for 2 h at 0° C. under nitrogen atmosphere. Then was stirred for overnight at room temperature under nitrogen atmosphere. The resulting mixture was diluted with ethyl acetate (500 mL). The mixture was washed with 1 N HCl (2×100 mL) and 2 N NaOH (2×200 mL) and brine (3×100 mL). The organic layer was concentrated under vacuum to afford tert-butyl (2S)-2-[methoxy(methyl)carbamoyl]azetidine-1-carboxylate (7.9 g, crude) as a colourless oil.

LC-MS: (M+H)⁺ found 245.2

### 78.2. Synthesis of tert-butyl (2S)-2-acetylazetidine-1-carboxylate

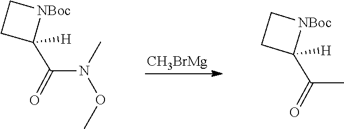

To a stirred solution of tert-butyl (2S)-2-[methoxy(methyl)carbamoyl]azetidine-1-carboxylate (7.4 g, 30.3 mmol, 1 equiv) in THF (150 mL) was added 1 M CH₃MgBr in THF (60 mL, 2 equiv) dropwise at −78° C. under nitrogen atmosphere. The resulting mixture was stirred for 2 h at −78° C. under nitrogen atmosphere. The reaction was quenched with sat. NH₄Cl (aq.) (300 mL) at 0° C. The mixture was extracted with EtOAc (3×100 mL) and concentrated under vacuum. The residue was purified by silica gel column chromatography, eluted with PE/EA (30:1) to afford tert-butyl (2S)-2-acetylazetidine-1-carboxylate (5 g, 82.84%) as an off-white oil.

LC-MS: (M−H)⁻ found 198.0

### 78.3. Synthesis of tert-butyl (2S)-2-[(1R)-1-hydroxyethyl]azetidine-1-carboxylate

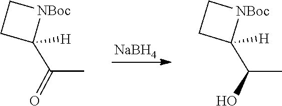

To a stirred solution of tert-butyl (2S)-2-acetylazetidine-1-carboxylate (4.5 g, 22.6 mmol, 1 equiv) in MeOH (80 mL) was added NaBH₄ (1.03 g, 27.1 mmol, 1.2 equiv) in portions at 0° C. under nitrogen atmosphere. The resulting mixture was stirred for 30 min at room temperature under nitrogen atmosphere. The reaction was quenched by the addition of sat. NH₄Cl (aq.) (80 mL) at 0° C. The mixture was extracted with EtOAc (3×50 mL). The organic layer was washed with brine (50 mL) and concentrated under vacuum. The residue was purified by silica gel column chromatography, eluted with PE/EA (30:1) to afford tert-butyl (2S)-2-[(1R)-1-hydroxyethyl]azetidine-1-carboxylate (2.5 g, 55.0%) as a colorless oil.

LC-MS: (M+H)⁺ found 202.0.

### 78.4. Synthesis of tert-butyl 2-[(1S)-1-[(4-bromopyridin-3-yl)oxy]ethyl]azetidine-1-carboxylate

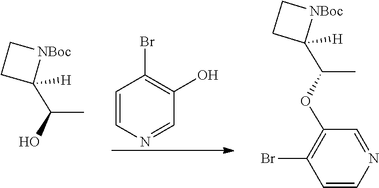

To a stirred solution of tert-butyl (2S)-2-[(1R)-1-hydroxyethyl]azetidine-1-carboxylate (1 g, 4.97 mmol, 1 equiv), 4-bromopyridin-3-ol (0.86 g, 4.97 mmol, 1 equiv) and PPh₃ (1.95 g, 7.454 mmol, 1.5 equiv) in THF (15 mL) was added DEAD (1.30 g, 7.46 mmol, 1.50 equiv) dropwise at 0° C. under nitrogen atmosphere. The resulting mixture was stirred overnight at room temperature under nitrogen atmosphere. The resulting mixture was concentrated under vacuum. The residue was purified by reverse flash chromatography with the following conditions: column, C18 silica gel; mobile phase, MeCN in water, 10% to 50% gradient in 20 min; detector, UV 254 nm. This resulted in tert-butyl 2-[(1S)-1-[(4-bromopyridin-3-yl)oxy]ethyl]azetidine-1-carboxylate (1.2 g, 67.6%) as a colorless oil.

LC-MS: (M+H)⁺ found 357.0.

### 78.5. Synthesis of tert-butyl (2R)-2-[(1S)-1-[(4-{4-oxo-1H,5H,6H,7H-pyrrolo[3,2-c]pyridin-2-yl}pyridin-3-yl)oxy]ethyl]azetidine-1-carboxylate

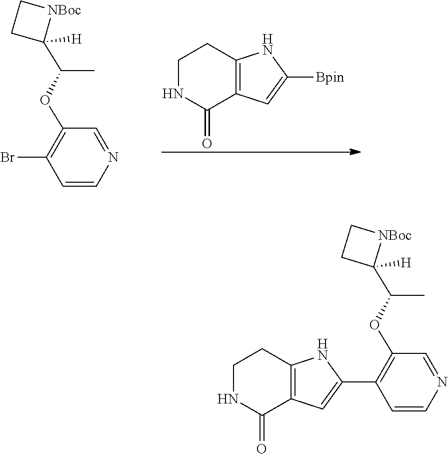

A mixture of tert-butyl 2-[(1S)-1-[(4-bromopyridin-3-yl)oxy]ethyl]azetidine-1-carboxylate (500 mg, 1.40 mmol, 1 equiv), 2-(4,4,5,5-tetramethyl-1,3,2-dioxaborolan-2-yl)-1H,5H,6H,7H-pyrrolo[3,2-c]pyridin-4-one (734 mg, 2.80 mmol, 2 equiv), Na₂CO₃ (445 mg, 4.20 mmol, 3 equiv) and 2nd Generation XPhos Precatalyst (110 mg, 0.140 mmol, 0.1 equiv) in dioxane (10 mL) and H₂O (1 mL) at room temperature under nitrogen atmosphere. The resulting mixture was stirred for 5 h at 80° C. under nitrogen atmosphere. The resulting mixture was concentrated under vacuum. The residue was purified by Prep-TLC (CH2Cl2/MeOH 15:1) to afford tert-butyl (2R)-2-[(1S)-1-[(4-{4-oxo-1H,5H,6H,7H-pyrrolo[3,2-c]pyridin-2-yl}pyridin-3-yl)oxy]ethyl]azetidine-1-carboxylate (450 mg, 77.9%) as a yellow oil.

LC-MS: (M+H)⁺ found 413.10.

### 78.6. Synthesis of tert-butyl (2R)-2-[(1S)-1-[(4-{3-iodo-4-oxo-1H,5H,6H,7H-pyrrolo[3,2-c]pyridin-2-yl}pyridin-3-yl)oxy]ethyl]azetidine-1-carboxylate

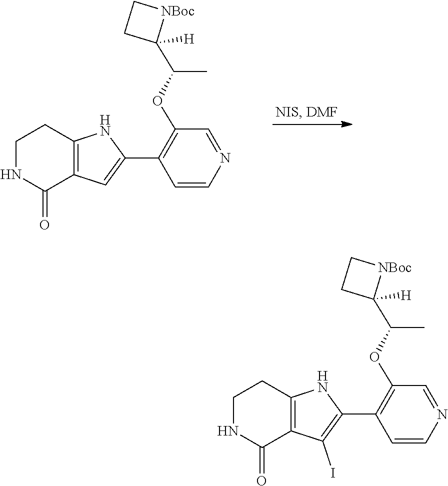 [SMILES: C[C@H](Oc1cnccc1-c1cc2c([nH]1)CCNC2=O)[C@H]1CCN1C(=O)OC(C)(C)C ; C[C@H](Oc1cnccc1-c1[nH]c2c(c1I)C(=O)NCC2)[C@H]1CCN1C(=O)OC(C)(C)C]

To a stirred solution of tert-butyl (2R)-2-[(1S)-1-[(4-{4-oxo-1H,5H,6H,7H-pyrrolo[3,2-c]pyridin-2-yl}pyridin-3-yl)oxy]ethyl]azetidine-1-carboxylate (410 mg, 0.994 mmol, 1 equiv) in DMF (5 mL) was added NIS (268 mg, 1.19 mmol, 1.2 equiv) in portions at 0° C. under nitrogen atmosphere. The resulting mixture was stirred for 1 h at room temperature under nitrogen atmosphere. The reaction was quenched with sat. sodium sulfite (aq.) (1 mL) at 0° C. The mixture was concentrated under vacuum. The residue was purified by silica gel column chromatography, eluted with CH2Cl2/MeOH (10:1) to afford tert-butyl (2R)-2-[(1S)-1-[(4-{3-iodo-4-oxo-1H,5H,6H,7H-pyrrolo[3,2-c]pyridin-2-yl}pyridin-3-yl)oxy]ethyl]azetidine-1-carboxylate (520 mg, 97.1%) as a yellow solid.

LC-MS: (M+H)⁺ found 539.05.

### 78.7. Synthesis of tert-butyl (2R)-2-[(1S)-1-[(4-{3-[(3-fluoro-2-methoxyphenyl)amino]-4-oxo-1H,5H,6H,7H-pyrrolo[3,2-c]pyridin-2-yl}pyridin-3-yl)oxy]ethyl]azetidine-1-carboxylate

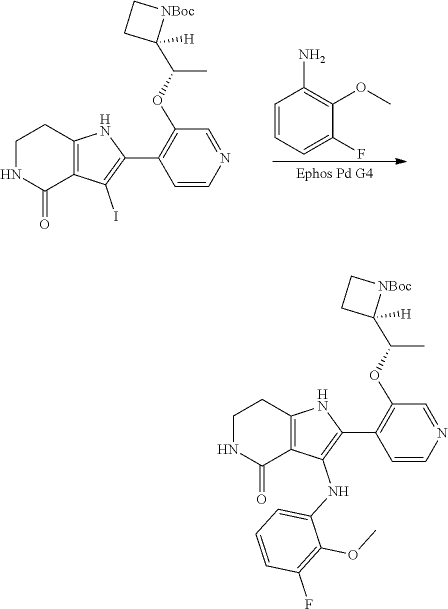

To a stirred mixture of tert-butyl (2R)-2-[(1S)-1-[(4-{3-iodo-4-oxo-1H,5H,6H,7H-pyrrolo[3,2-c]pyridin-2-yl}pyridin-3-yl)oxy]ethyl]azetidine-1-carboxylate (470 mg, 0.873 mmol, 1 equiv) and 3-fluoro-2-methoxyaniline (246 mg, 1.74 mmol, 2 equiv) in DMF (8 mL) were added Cs₂CO₃ (568 mg, 1.74 mmol, 2 equiv) and EPhos Pd G4 (80.2 mg, 0.087 mmol, 0.1 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl (2R)-2-[(1S)-1-[(4-{3-[(3-fluoro-2-methoxyphenyl)amino]-4-oxo-1H,5H,6H,7H-pyrrolo[3,2-c]pyridin-2-yl}pyridin-3-yl)oxy]ethyl]azetidine-1-carboxylate (480 mg, 99.6%) as a yellow solid.

LC-MS: (M+H)⁺ found 552.20.

### 78.8. Synthesis of 2-{3-[(1S)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one

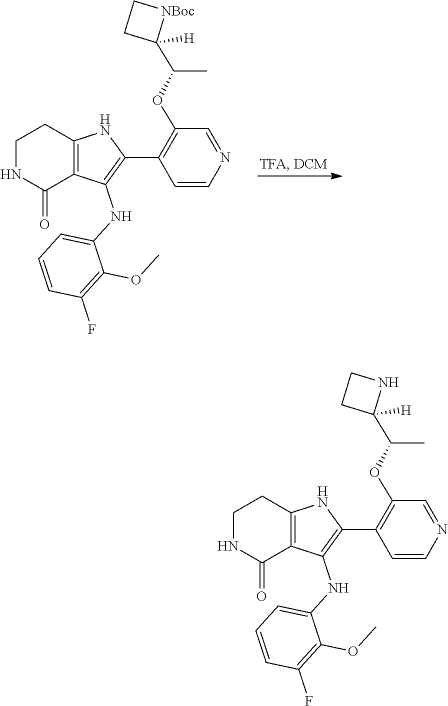 [SMILES: COc1c(F)cccc1Nc1c(-c2ccncc2O[C@@H](C)[C@H]2CCN2C(=O)OC(C)(C)C)[nH]c2c1C(=O)NCC2 ; COc1c(F)cccc1Nc1c(-c2ccncc2O[C@@H](C)[C@H]2CCN2)[nH]c2c1C(=O)NCC2]

To a stirred solution of tert-butyl (2R)-2-[(1S)-1-[(4-{3-[(3-fluoro-2-methoxyphenyl)amino]-4-oxo-1H,5H,6H,7H-pyrrolo[3,2-c]pyridin-2-yl}pyridin-3-yl)oxy]ethyl]azetidine-1-carboxylate (100 mg, 0.181 mmol, 1 equiv) in DCM (3 mL) was added TFA (1 mL) at room temperature under nitrogen atmosphere. The resulting mixture was stirred for 1 h and concentrated under reduced pressure to afford 2-{3-[(1S)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one (120 mg, crude) as a yellow oil.

LC-MS: (M+H)⁺ found 452.20.

### 78.9. Synthesis of 3-[(3-fluoro-2-methoxyphenyl)amino]-2-{3-[(1S)-1-[(2R)-1-(prop-2-enoyl)azetidin-2-yl]ethoxy]pyridin-4-yl}-1H,5H,6H,7H-pyrrolo[3,2-c]pyridin-4-one

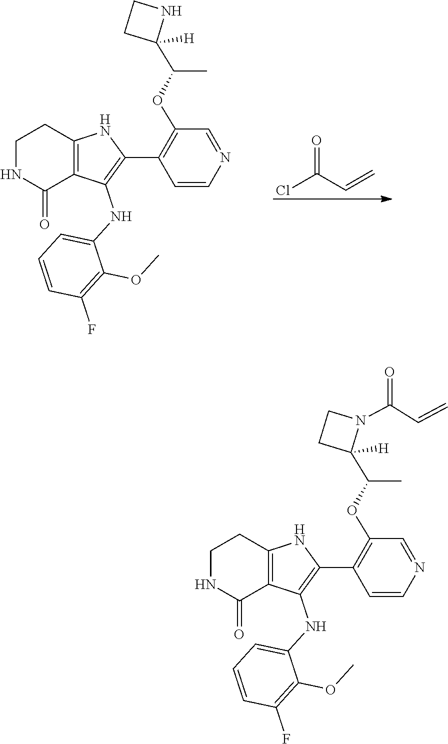

A mixture of 2-{3-[(1S)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one (150 mg, 0.332 mmol, 1 equiv) in THF (3 mL) was basified to pH 8 with saturated NaHCO₃(aq.). To the above mixture was added acryloyl chloride (45.1 mg, 0.498 mmol, 1.5 equiv) dropwise at 0° C. The resulting mixture was stirred for 30 min at room temperature. The reaction was quenched with MeOH (2 mL) at 0° C. and extracted with CH2Cl2/MeOH (10/1) (2×10 mL), dried over anhydrous Na₂SO₄ and filtered. The filtrate was concentrated under reduced pressure to give crude product (120 mg) that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 22% B to 42% B in 9 min, 42% B; Wave Length: 254/220 nm; RT1(min): 7.53;) to afford 3-[(3-fluoro-2-methoxyphenyl)amino]-2-{3-[(1S)-1-[(2R)-1-(prop-2-enoyl)azetidin-2-yl]ethoxy]pyridin-4-yl}-1H,5H,6H,7H-pyrrolo[3,2-c]pyridin-4-one (22.3 mg, 13.2%) as a yellow solid.

LC-MS: (M+H)⁺ found 506.15.

¹H NMR (400 MHz, Chloroform-d) δ 11.80 (s, 1H), 8.26 (s, 1H), 7.94 (d, 1H), 7.62 (s, 1H), 7.57 (d, 1H), 6.65-6.56 (m, 1H), 6.50-6.28 (m, 3H), 6.12-6.07 (m, 1H), 5.85-5.78 (m, 1H), 5.22 (s, 1H), 5.02-4.98 (m, 1H), 4.78-4.69 (m, 1H), 4.30-4.18 (m, 2H), 4.13 (d, 3H), 3.64-3.52 (m, 2H), 3.24-3.07 (m, 2H), 2.65-2.54 (m, 1H), 2.19-2.07 (m, 1H), 1.60 (d, 3H).

### Example 79. 2-{3-[(1S)-1-[(2R)-1-(but-2-ynoyl)azetidin-2-yl]ethoxy]pyridin-4-yl}-3-[(3-fluoro-2-methoxyphenyl)amino]-1H,5H,6H,7H-pyrrolo[3,2-c]pyridin-4-one (compound 313)

### 79.1. Synthesis of 2-{3-[(1S)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one

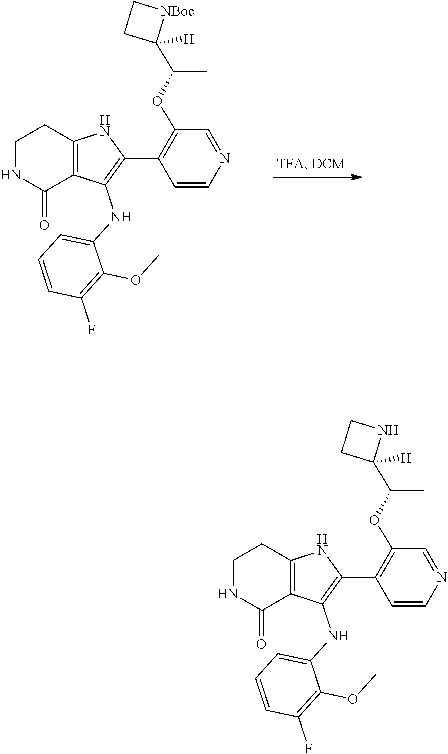 [SMILES: COc1c(F)cccc1Nc1c(-c2ccncc2O[C@@H](C)[C@H]2CCN2C(=O)OC(C)(C)C)[nH]c2c1C(=O)NCC2 ; COc1c(F)cccc1Nc1c(-c2ccncc2O[C@@H](C)[C@H]2CCN2)[nH]c2c1C(=O)NCC2]

To a stirred solution of tert-butyl (2R)-2-[(1S)-1-[(4-{3-[(3-fluoro-2-methoxyphenyl)amino]-4-oxo-1H,5H,6H,7H-pyrrolo[3,2-c]pyridin-2-yl}pyridin-3-yl)oxy]ethyl]azetidine-1-carboxylate (100 mg, 0.181 mmol, 1 equiv) in DCM (3 mL) was added TFA (1 mL) at room temperature under nitrogen atmosphere. The resulting mixture was stirred for 1 h and concentrated under reduced pressure to afford 2-{3-[(1S)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one (120 mg, crude) as a yellow oil.

LC-MS: (M+H)⁺ found 452.20.

### 79.2. Synthesis of 2-{3-[(1S)-1-[(2R)-1-(but-2-ynoyl)azetidin-2-yl]ethoxy]pyridin-4-yl}-3-[(3-fluoro-2-methoxyphenyl)amino]-1H,5H,6H,7H-pyrrolo[3,2-c]pyridin-4-one

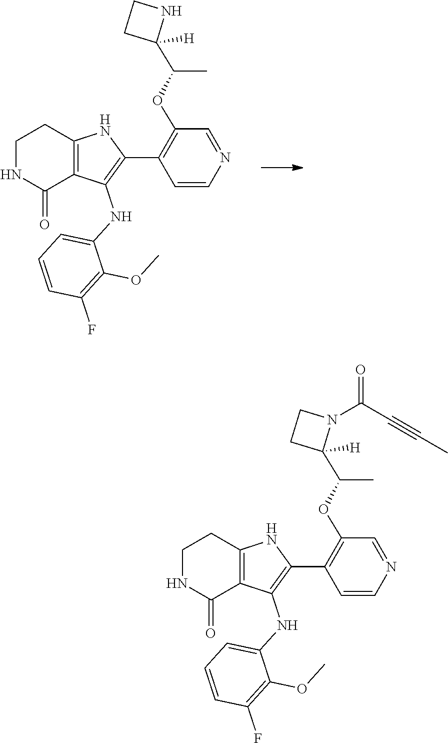 [SMILES: COc1c(F)cccc1Nc1c(-c2ccncc2O[C@@H](C)[C@H]2CCN2)[nH]c2c1C(=O)NCC2 ; CC#CC(=O)N1CC[C@@H]1[C@H](C)Oc1cnccc1-c1[nH]c2c(c1Nc1cccc(F)c1OC)C(=O)NCC2]

To a stirred solution of 2-{3-[(1S)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one (100 mg, 0.221 mmol, 1 equiv) in THF (3 mL) was basified to pH 8 with DIEA. To the above mixture was added 2-butynoic acid (27.9 mg, 0.332 mmol, 1.5 equiv) at 0° C. under nitrogen atmosphere followed by the addition of T3P (282 mg, 0.442 mmol, 3 equiv, 50% in EA) dropwise. The resulting mixture was stirred for 1 h at room temperature under nitrogen atmosphere. Saturated aqueous NaHCO₃ (10 mL) was added to the reaction mixture at 0° C. and extracted with EtOAc (3×10 mL), dried over anhydrous Na₂SO₄ and filtered. The filtrate was concentrated under reduced pressure to give crude product (120 mg) that was purified by Prep-HPLC under the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 20% B to 50% B in 8 min, 50% B; Wave Length: 220/254 nm; RT1(min): 8.00;) to afford 2-{3-[(1S)-1-[(2R)-1-(but-2-ynoyl)azetidin-2-yl]ethoxy]pyridin-4-yl}-3-[(3-fluoro-2-methoxyphenyl)amino]-1H,5H,6H,7H-pyrrolo[3,2-c]pyridin-4-one (31.4 mg, 27.09%) as a yellow solid.

LC-MS: (M+H)⁺ found 518.15.

¹H NMR (400 MHz, Chloroform-d) δ 11.47 (s, 1H), 8.26 (s, 1H), 7.93 (d, 1H), 7.69 (s, 1H), 7.54 (d, 1H), 6.62-6.58 (m, 1H), 6.56-6.46 (m, 1H), 6.06-6.04 (m, 1H), 5.23 (s, 1H), 4.95-4.91 (m, 1H), 4.74-4.69 (m, 1H), 4.27-4.21 (m, 1H), 4.19-4.09 (m, 4H), 3.59-3.53 (m, 2H), 3.17-3.04 (m, 2H), 2.62-2.51 (m, 1H), 2.09-2.02 (m, 4H), 1.59 (d, 3H).

### Example 80. 2-(3-{[(2S)-4,4-dimethyl-1-(prop-2-enoyl)pyrrolidin-2-yl]methoxy}pyridin-4-yl)-3-[(3-fluoro-2-methoxyphenyl)amino]-1H,5H,6H,7H-pyrrolo[3,2-c]pyridin-4-one (compound 327)

### 80.1. Synthesis of tert-butyl (2S)-2-{[(4-{3-[(3-fluoro-2-methoxyphenyl) amino]-4-oxo-1H,5H,6H,7H-pyrrolo[3,2-c] pyridin-2-yl} pyridin-3-yl) oxy] methyl}-4,4-dimethylpyrrolidine-1-carboxylate

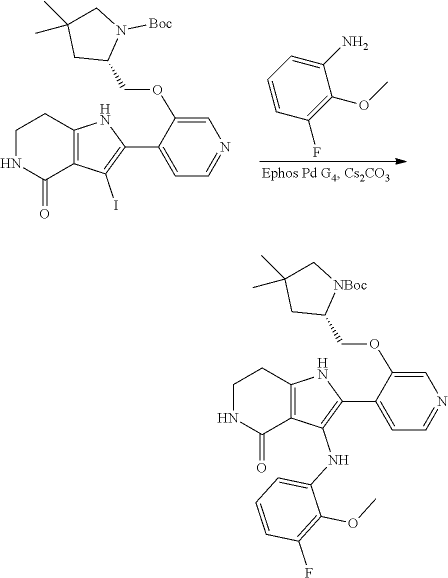

To a stirred solution of tert-butyl (2S)-2-{[(4-{3-iodo-4-oxo-1H,5H,6H,7H-pyrrolo[3,2-c]pyridin-2-yl} pyridin-3-yl) oxy] methyl}-4,4-dimethylpyrrolidine-1-carboxylate (170 mg, 0.300 mmol, 1 equiv) and Cs₂CO₃ (196 mg, 0.600 mmol, 2 equiv) in DMF (2 mL) were added EPhos Pd G4 (27.6 mg, 0.030 mmol, 0.1 equiv) and 3-fluoro-2-methoxyaniline (50.8 mg, 0.360 mmol, 1.2 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl (2S)-2-{[(4-{3-[(3-fluoro-2-methoxyphenyl) amino]-4-oxo-1H,5H,6H,7H-pyrrolo[3,2-c] pyridin-2-yl}pyridin-3-yl) oxy] methyl}-4,4-dimethylpyrrolidine-1-carboxylate (150 mg, 86.22%) as a yellow solid.

LC-MS: M+H found: 580.0.

### 80.2. Synthesis of 2-(3-{[(2S)-4,4-dimethylpyrrolidin-2-yl] methoxy} pyridin-4-yl)-3-[(3-fluoro-2-methoxyphenyl) amino]-1H,5H,6H,7H-pyrrolo[3,2-c] pyridin-4-one

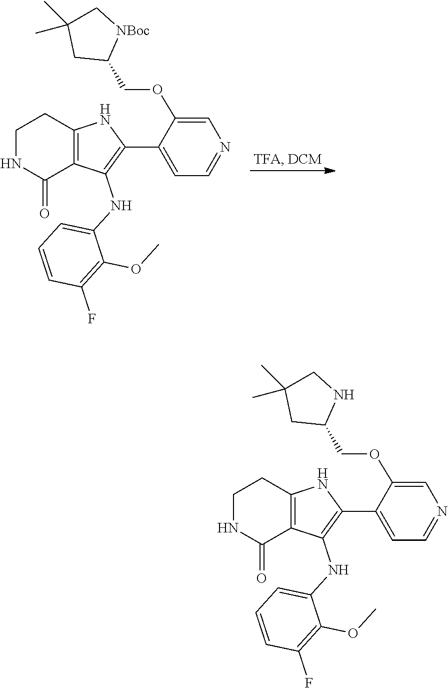 [SMILES: COc1c(F)cccc1Nc1c(-c2ccncc2OC[C@@H]2CC(C)(C)CN2C(=O)OC(C)(C)C)[nH]c2c1C(=O)NCC2 ; COc1c(F)cccc1Nc1c(-c2ccncc2OC[C@@H]2CC(C)(C)CN2)[nH]c2c1C(=O)NCC2]

To a stirred solution of tert-butyl (2S)-2-{[(4-{3-[(3-fluoro-2-methoxyphenyl) amino]-4-oxo-1H,5H,6H,7H-pyrrolo[3,2-c] pyridin-2-yl} pyridin-3-yl) oxy] methyl}-4,4-dimethylpyrrolidine-1-carboxylate (120 mg, 0.207 mmol, 1 equiv) in DCM (1 mL) was added TFA (0.3 mL) at room temperature under nitrogen atmosphere. The resulting mixture was stirred for 1 h and concentrated under reduced pressure to afford 2-(3-{[(2S)-4,4-dimethylpyrrolidin-2-yl] methoxy} pyridin-4-yl)-3-[(3-fluoro-2-methoxyphenyl) amino]-1H,5H,6H,7H-pyrrolo[3,2-c] pyridin-4-one (150 mg, crude) as a red oil.

LC-MS: M+H found: 480.

### 80.3. Synthesis of 2-(3-{[(2S)-4,4-dimethyl-1-(prop-2-enoyl)pyrrolidin-2-yl]methoxy}pyridin-4-yl)-3-[(3-fluoro-2-methoxyphenyl)amino]-1H,5H,6H,7H-pyrrolo[3,2-c]pyridin-4-one

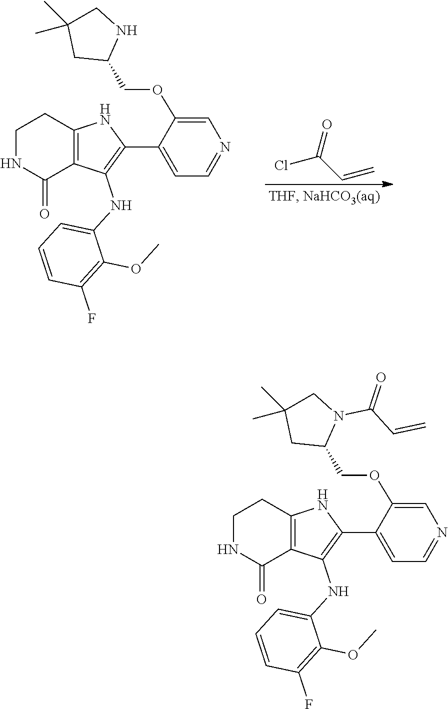

To a stirred solution of 2-(3-{[(2S)-4,4-dimethylpyrrolidin-2-yl]methoxy}pyridin-4-yl)-3-[(3-fluoro-2-methoxyphenyl)amino]-1H,5H,6H,7H-pyrrolo[3,2-c]pyridin-4-one (150 mg, 0.250 mmol, 1 equiv) in THF (1.5 mL) was basified to pH 8 with sat. NaHCO₃(aq.). To the above mixture was added acryloyl chloride (26.1 mg, 0.287 mmol, 1.15 equiv) at 0° C. under nitrogen atmosphere. The resulting mixture was stirred for 1 h at room temperature under nitrogen atmosphere. MeOH (1 mL) was added to the reaction mixture at 0° C. and extracted with CH₂Cl₂ (3×5 mL), dried over anhydrous Na₂SO₄ and filtered. The filtrate was concentrated under reduced pressure to give crude product (130 mg) that was purified by Prep-HPLC under the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 28% B to 58% B in 10 min, 58% B; Wave Length: 220/254 nm; RT1(min): 10.37; Number Of Runs: 0) to afford 2-(3-{[(2S)-4,4-dimethyl-1-(prop-2-enoyl)pyrrolidin-2-yl]methoxy}pyridin-4-yl)-3-[(3-fluoro-2-methoxyphenyl)amino]-1H,5H,6H,7H-pyrrolo[3,2-c]pyridin-4-one (25.1 mg, 18.67%) as a yellow solid.

LC-MS: M+H found: 534.15.

¹H NMR (400 MHz, Chloroform-d) δ 11.67 (s, 1H), 8.24 (s, 1H), 7.99 (s, 1H), 7.72-7.47 (m, 2H), 6.70-6.44 (m, 3H), 6.38-4.35 (m, 1H), 6.11-6.08 (m, 1H), 5.78-5.72 (m, 1H), 5.28 (s, 1H), 5.07-4.98 (m, 1H), 4.26 (t, 1H), 4.26-4.03 (m, 4H), 3.78-3.47 (m, 3H), 3.35 (d, 1H), 3.30-3.12 (m, 2H), 2.31-1.92 (m, 1H), 1.62-1.39 (m, 1H), 1.25 (s, 3H), 1.07 (s, 3H).

Example 81. 2-(3-{2-[(2R)-1-[(2E)-4-(dimethylamino)but-2-enoyl]-4,4-dimethylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one (compound 344)

### 81.1. Synthesis of 2-(3-{2-[(2R)-4,4-dimethylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one

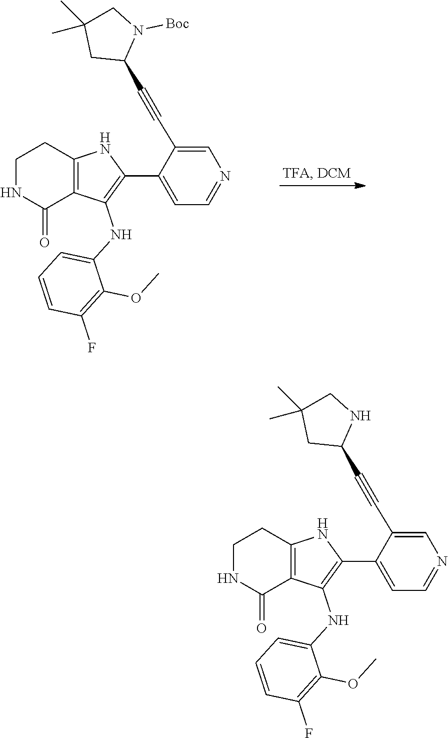 [SMILES: COc1c(F)cccc1Nc1c(-c2ccncc2C#C[C@H]2CC(C)(C)CN2C(=O)OC(C)(C)C)[nH]c2c1C(=O)NCC2 ; COc1c(F)cccc1Nc1c(-c2ccncc2C#C[C@H]2CC(C)(C)CN2)[nH]c2c1C(=O)NCC2]

To a stirred solution of tert-butyl (2R)-2-[2-(4-{3-[(3-fluoro-2-methoxyphenyl) amino]-4-oxo-1H,5H,6H,7H-pyrrolo[3,2-c] pyridin-2-yl} pyridin-3-yl) ethynyl]-4,4-dimethylpyrrolidine-1-carboxylate (70 mg, 0.122 mmol, 1.0 equiv) in DCM (1 mL) was added TFA (0.3 mL) at room temperature under nitrogen atmosphere. The resulting mixture was stirred for 1 h and concentrated under reduced pressure to afford 2-(3-{2-[(2R)-4,4-dimethylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one (70 mg, crude) as a red oil.

LC-MS: (M+H)⁺ found 474.10.

### 81.2. Synthesis of 2-(3-{2-[(2R)-1-[(2E)-4-(dimethylamino)but-2-enoyl]-4,4-dimethylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one

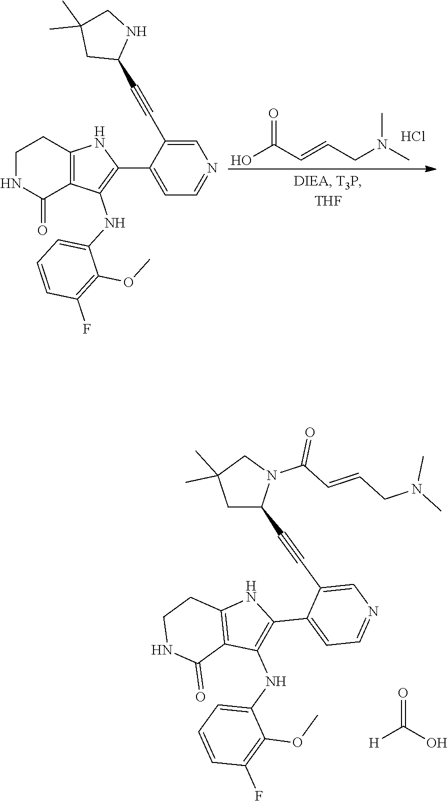

To a stirred solution of 2-(3-{2-[(2R)-4,4-dimethylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one (70 mg, 0.148 mmol, 1.00 equiv) in THF (1 mL) was basified to pH 8 with DIEA. To the above mixture was added (2E)-4-(dimethylamino)but-2-enoic acid (28.5 mg, 0.222 mmol, 1.50 equiv) and T₃P (93.3 mg, 0.296 mmol, 2.00 equiv, 50% in EA) dropwise. The resulting mixture was stirred for 1 h at room temperature under nitrogen atmosphere. Saturated aqueous NaHCO₃(10 mL) was added to the reaction mixture at 0° C. and extracted with EtOAc (3×10 mL), dried over anhydrous Na₂SO₄ and filtered. The filtrate was concentrated under reduced pressure to give crude product (60 mg) that was purified by Prep-HPLC under the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 17% B to 34% B in 7 min, 34% B; Wave Length: 254/220 nm; RT1(min): 7; Number Of Runs: 0) to afford 2-(3-{2-[(2R)-1-[(2E)-4-(dimethylamino)but-2-enoyl]-4,4-dimethylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one (46.1 mg, 52.64%) as an off-white solid.

LC-MS: (M+H)⁺ found 585.15.

¹H NMR (400 MHz, Chloroform-d) δ 11.18 (s, 1H), 8.51 (s, 1H), 8.13 (d, 1H), 7.67 (s, 1H), 7.46 (d, 1H), 6.98-6.85 (m, 1H), 6.58-6.32 (m, 3H), 6.11-6.03 (m, 1H), 5.33 (s, 1H), 4.90 (t, 1H), 4.07 (s, 3H), 3.66-3.37 (m, 4H), 3.27-3.14 (m, 4H), 2.35 (s, 6H), 2.21-2.15 (m, 1H), 2.09-2.03 (m, 1H), 1.32 (s, 3H), 1.13 (s, 3H).

### Example 82. 2-(3-{[(2R)-1-(but-2-ynoyl)azetidin-2-yl]methoxy}pyridin-4-yl)-3-[(3,4-difluoro-2-methoxyphenyl)amino]-1H,5H,6H,7H-pyrrolo[3,2-c]pyridin-4-one (compound 351)

### 82.1. Synthesis of tert-butyl (2R)-2-{[(4-{3-[(3,4-difluoro-2-methoxyphenyl) amino]-4-oxo-1H,5H,6H,7H-pyrrolo[3,2-c] pyridin-2-yl} pyridin-3-yl) oxy] methyl}azetidine-1-carboxylate

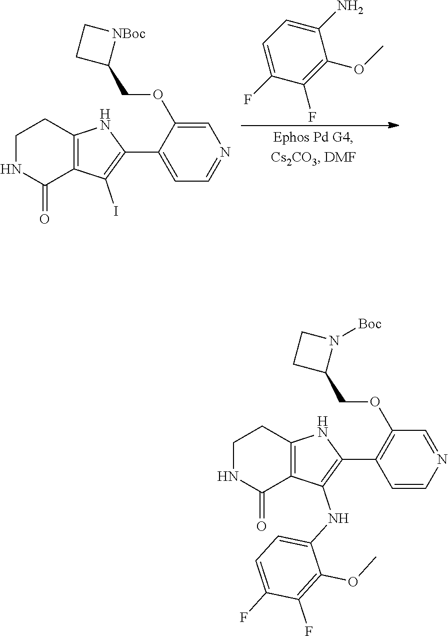

To a stirred mixture of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (240 mg, 0.46 mmol, 1.00 equiv) and 3,4-difluoro-2-methoxyaniline (87 mg, 0.55 mmol, 1.20 equiv) in DMF (4 mL) were added Ephos Pd G4 (42 mg, 0.046 mmol, 0.10 equiv) and Cs₂CO₃ (300 mg, 0.92 mmol, 2.00 equiv) under argon atmosphere. The resulting mixture was stirred for 2 h at 50° C. under argon atmosphere. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl (2R)-2-{[(4-{3-[(3,4-difluoro-2-methoxyphenyl) amino]-4-oxo-1H,5H,6H,7H-pyrrolo[3,2-c] pyridin-2-yl} pyridin-3-yl) oxy] methyl} azetidine-1-carboxylate (200 mg, 75.86%) as a yellow solid.

LC-MS: (M+H)⁺ found 556.55.

### 82.2. Synthesis of 2-{3-[(2R)-azetidin-2-ylmethoxy]pyridin-4-yl}-3-[(3,4-difluoro-2-methoxyphenyl)amino]-1H,5H,6H,7H-pyrrolo[3,2-c]pyridin-4-one

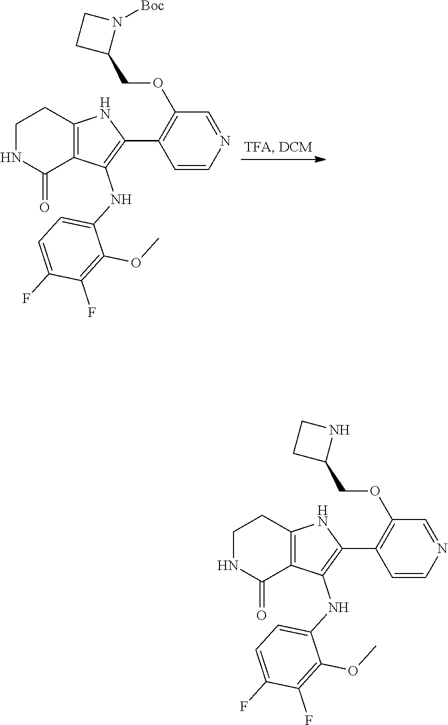 [SMILES: COc1c(Nc2c(-c3ccncc3OC[C@H]3CCN3C(=O)OC(C)(C)C)[nH]c3c2C(=O)NCC3)ccc(F)c1F ; COc1c(Nc2c(-c3ccncc3OC[C@H]3CCN3)[nH]c3c2C(=O)NCC3)ccc(F)c1F]

To a stirred solution of tert-butyl (2R)-2-{[(4-{3-[(3,4-difluoro-2-methoxyphenyl) amino]-4-oxo-1H,5H,6H,7H-pyrrolo[3,2-c] pyridin-2-yl} pyridin-3-yl) oxy] methyl}azetidine-1-carboxylate (90 mg, 0.162 mmol, 1.00 equiv) in DCM (1 mL) was added TFA (0.3 mL) at room temperature under nitrogen atmosphere. The resulting mixture was stirred for 1 h and concentrated under reduced pressure to afford 2-{3-[(2R)-azetidin-2-ylmethoxy]pyridin-4-yl}-3-[(3,4-difluoro-2-methoxyphenyl)amino]-1H,5H,6H,7H-pyrrolo[3,2-c]pyridin-4-one (180 mg, crude) as a red oil.

LC-MS: (M+H)⁺ found 456.05.

### 82.3. Synthesis of 2-(3-{[(2R)-1-(but-2-ynoyl)azetidin-2-yl]methoxy}pyridin-4-yl)-3-[(3,4-difluoro-2-methoxyphenyl)amino]-1H,5H,6H,7H-pyrrolo[3,2-c]pyridin-4-one

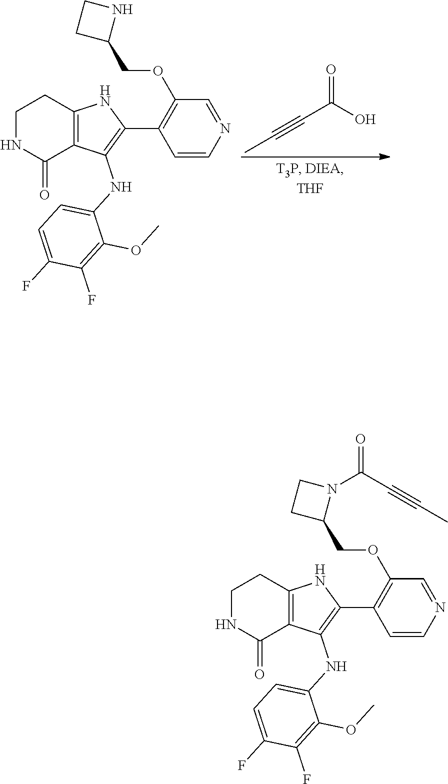

To a stirred mixture of 2-{3-[(2R)-azetidin-2-ylmethoxy]pyridin-4-yl}-3-[(3,4-difluoro-2-methoxyphenyl)amino]-1H,5H,6H,7H-pyrrolo[3,2-c]pyridin-4-one (90 mg, 0.198 mmol, 1.00 equiv) in THF (2 mL) was basified to pH 8 with DIEA. To the above mixture was added 2-butynoic acid (24.8 mg, 0.297 mmol, 1.50 equiv) at 0° C. under nitrogen atmosphere followed by the addition of T₃P (125 mg, 0.396 mmol, 2.0 equiv, 50% in EA) dropwise. The resulting mixture was stirred for 1 h at room temperature under nitrogen atmosphere. Saturated aqueous NaHCO₃(10 mL) was added to the reaction mixture at 0° C. and extracted with EtOAc (3×10 mL), dried over anhydrous Na₂SO₄ and filtered. The filtrate was concentrated under reduced pressure to give crude product (60 mg) that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 37% B to 65% B in 8 min, 65% B; Wave Length: 254/220 nm; RT1(min): 5.78; Number Of Runs: 0) to afford 2-(3-{[(2R)-1-(but-2-ynoyl)azetidin-2-yl]methoxy}pyridin-4-yl)-3-[(3,4-difluoro-2-methoxyphenyl)amino]-1H,5H,6H,7H-pyrrolo[3,2-c]pyridin-4-one (30.3 mg, 29.28%) as off-white solid.

LC-MS: (M+H)⁺ found 522.10.

¹H NMR (400 MHz, Chloroform-d) δ 11.31 (s, 1H), 8.17 (s, 1H), 7.94 (d, 1H), 7.41 (d, 1H), 7.29 (s, 1H), 6.46-6.33 (m, 1H), 5.93-5.84 (m, 1H), 5.14 (s, 1H), 4.92-4.79 (m, 1H), 4.42 (t, 1H), 4.26-4.13 (m, 3H), 4.06 (s, 3H), 3.53-3.46 (m, 2H), 3.16-2.94 (m, 2H), 2.60-2.50 (m, 1H), 2.11-2.01 (m, 1H), 1.96 (s, 3H).

### Example 83. 2-(3-{2-[(1S,3S,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one (compound 516)

### 83.1. Synthesis of tert-butyl (1S,3S,5S)-3-(hydroxymethyl)-2-azabicyclo[3.1.0]hexane-2-carboxylate

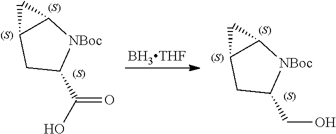

A mixture of (1S,3S,5S)-2-(tert-butoxycarbonyl)-2-azabicyclo[3.1.0]hexane-3-carboxylic acid (0.5 g, 2.20 mmol, 1 equiv) and BH₃-THF (4.40 mL, 4.400 mmol, 2 equiv) in THF (5 mL) was stirred for 1 h at 0° C. under nitrogen atmosphere. Desired product could be detected by TLC. The resulting mixture was extracted with CH₂Cl₂ (2×100 mL). The combined organic layers were washed with water (50 mL), dried over anhydrous Na₂SO₄. After filtration, the filtrate was concentrated under reduced pressure. The residue was purified by silica gel column chromatography, eluted with PE/EA (10:1) to afford tert-butyl (1S,3S,5S)-3-(hydroxymethyl)-2-azabicyclo[3.1.0]hexane-2-carboxylate (0.45 g, 95.9%) as a colorless oil.

### 83.2. Synthesis of tert-butyl (1S,3S,5S)-3-formyl-2-azabicyclo[3.1.0]hexane-2-carboxylate

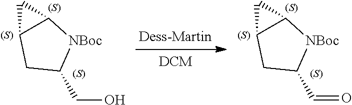

A mixture of tert-butyl (1S,3S,5S)-3-(hydroxymethyl)-2-azabicyclo[3.1.0]hexane-2-carboxylate (0.45 g, 2.11 mmol, 1 equiv) and Dess-Martin (0.94 g, 2.21 mmol, 1.05 equiv) in DCM (10 mL) was stirred for 2 h at room temperature under nitrogen atmosphere. Desired product could be detected by TLC. The resulting mixture was extracted with CH₂Cl₂ (2×100 mL). The combined organic layers were washed with water (50 mL), dried over anhydrous Na₂SO₄. After filtration, the filtrate was concentrated under reduced pressure. The residue was purified by silica gel column chromatography, eluted with PE/EA (10:1) to afford tert-butyl (1S,3S,5S)-3-formyl-2-azabicyclo[3.1.0]hexane-2-carboxylate (390 mg, 87.49%) as a colorless oil.

### 83.3. Synthesis of tert-butyl (1S,3S,5S)-3-ethynyl-2-azabicyclo[3.1.0]hexane-2-carboxylate

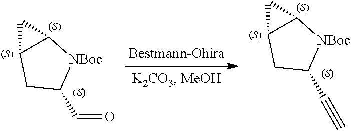

To a stirred mixture of tert-butyl (1S,3S,5S)-3-formyl-2-azabicyclo[3.1.0]hexane-2-carboxylate (390 mg, 1.84 mmol, 1 equiv) and K₂CO₃ (510 mg, 3.69 mmol, 2 equiv) in MeOH (4 mL) was added dimethyl (1-diazo-2-oxopropyl)phosphonate (425 mg, 2.21 mmol, 1.2 equiv) in portions at 0° C. under nitrogen atmosphere. The resulting mixture was stirred for overnight at room temperature under nitrogen atmosphere. Desired product could be detected by TLC. The reaction was quenched with sat. potassium sodium tartrate (aq.) at room temperature. The resulting mixture was extracted with EtOAc (2×200 mL). The combined organic layers were washed with water (100 mL), dried over anhydrous Na₂SO₄. After filtration, the filtrate was concentrated under reduced pressure. The residue was purified by silica gel column chromatography, eluted with PE/EA (10:1) to afford tert-butyl (1S,3S,5S)-3-ethynyl-2-azabicyclo[3.1.0]hexane-2-carboxylate (300 mg, 78.4%) as a light yellow oil.

### 83.4. Synthesis of tert-butyl (1S,3S,5S)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate

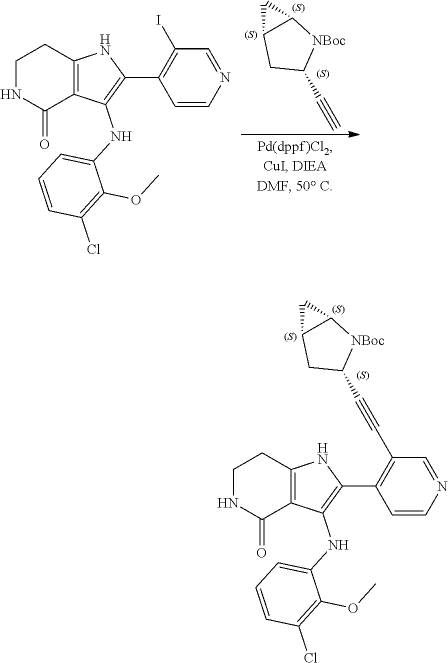

To a stirred mixture of 3-[(3-chloro-2-methoxyphenyl)amino]-2-(3-iodopyridin-4-yl)-1H,5H,6H,7H-pyrrolo[3,2-c]pyridin-4-one (130 mg, 0.26 mmol, 1 equiv) tert-butyl (1S,3S,5S)-3-ethynyl-2-azabicyclo[3.1.0]hexane-2-carboxylate (163 mg, 0.78 mmol, 3 equiv) and DIEA (169 mg, 1.31 mmol, 5 equiv) in DMF (2 mL) were added CuI (25 mg, 0.13 mmol, 0.5 equiv) and Pd(dppf)Cl₂·CH₂Cl₂ (32 mg, 0.039 mmol, 0.15 equiv) in portions at room temperature under argon atmosphere. The resulting mixture was stirred for 2 h at 50° C. under argon atmosphere. Desired product could be detected by LCMS. The resulting mixture was concentrated under reduced pressure. The residue was purified by silica gel column chromatography, eluted with CH₂Cl₂/MeOH (20:1) to afford tert-butyl (1S,3S,5S)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (110 mg, 72.92%) as a yellow solid.

LC-MS: [M+H]+ found 574.00.

### 83.5. Synthesis of 2-(3-{2-[(1S,3S,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one

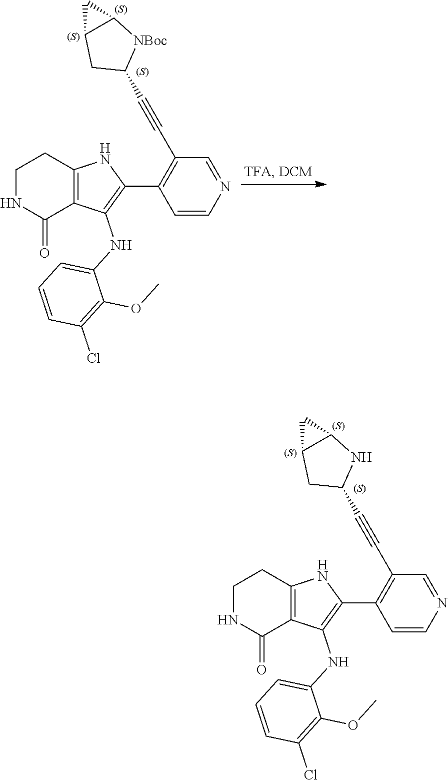

A mixture of tert-butyl (1S,3S,5S)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (110 mg, 0.19 mmol, 1 equiv) in TFA (1 mL) and DCM (1 mL) was stirred for 1 h at room temperature under air atmosphere. Desired product could be detected by LCMS. The resulting mixture was concentrated under reduced pressure. This resulted in 2-(3-{2-[(1S,3S,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one (80 mg, 88.09%) as a yellow oil.

LC-MS: [M+H]+ found 474.00.

### 83.6. Synthesis of 3-[(3-chloro-2-methoxyphenyl)amino]-2-(3-{2-[(1S,3S,5S)-2-(prop-2-enoyl)-2-azabicyclo[3.1.0]hexan-3-yl]ethynyl}pyridin-4-yl)-1H,5H,6H,7H-pyrrolo[3,2-c]pyridin-4-one

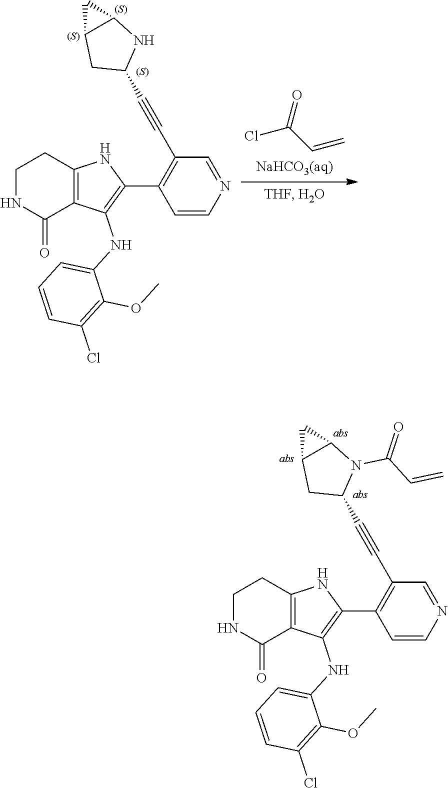

A mixture of 2-(3-{2-[(1S,3S,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one (70 mg, 0.14 mmol, 1 equiv) and acryloyl chloride (12 mg, 0.13 mmol, 0.9 equiv) in THF (1 mL) and sat. NaHCO₃(aq.) (1 mL) was stirred for 1 h at 0° C. under air atmosphere. Desired product could be detected by LCMS. The resulting mixture was concentrated under reduced pressure. The crude product was purified by prep-HPLC with the following conditions (Column: Xselect CSH F-Phenyl OBD column, 30*250 mm, 5 μm; Mobile Phase A: Water (0.1% FA), Mobile Phase B: ACN; Flow rate: 60 mL/min; Gradient: 19% B to 49% B in 7 min; Wave Length: 254 nm; RT1(min): 6.5; Number Of Runs: 0) to afford 3-[(3-chloro-2-methoxyphenyl)amino]-2-(3-{2-[(1S,3S,5S)-2-(prop-2-enoyl)-2-azabicyclo[3.1.0]hexan-3-yl]ethynyl}pyridin-4-yl)-1H,5H,6H,7H-pyrrolo[3,2-c]pyridin-4-one (24.4 mg, 31.20%) as a yellow solid.

LC-MS: [M+H]⁺ found 454.93.

¹H NMR (300 MHz, Chloroform-d) δ 11.08 (s, 1H), 8.52 (s, 1H), 8.08 (d, J=5.9 Hz, 1H), 7.92 (s, 1H), 7.39 (d, J=5.9 Hz, 1H), 6.80-6.70 (m, 2H), 6.62 (t, J=8.1 Hz, 1H), 6.43-6.37 (m, 1H), 6.20-6.17 (m, 1H), 5.82-5.78 (m, 1H), 5.29-5.23 (m, 2H), 4.07 (s, 3H), 3.64-3.55 (m, 3H), 3.29 (t, J=6.7 Hz, 2H), 2.78-2.68 (m, 1H), 2.39-2.33 (m, 1H), 1.99-1.68 (m, 1H), 1.12-1.06 (m, 2H).

### Example 84. 2-(3-{[(3R)-4-[(2E)-4-(dimethylamino)but-2-enoyl]morpholin-3-yl]methoxy}pyridin-4-yl)-3-[(3-fluoro-2-methoxyphenyl)amino]-1H,5H,6H,7H-pyrrolo[3,2-c]pyridin-4-one)(compound 196)

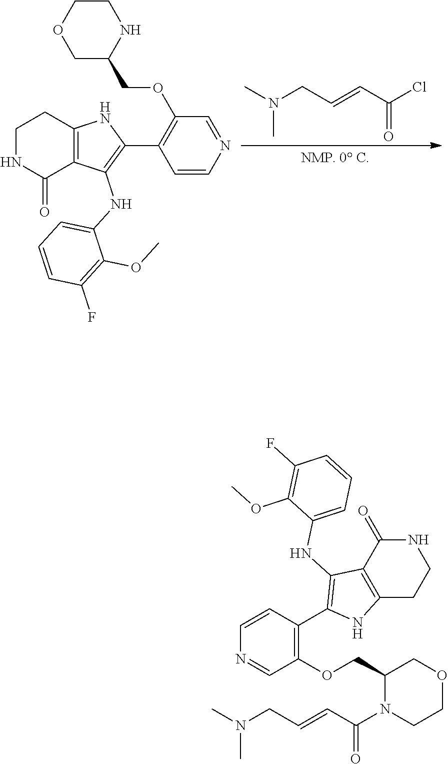

To a stirred mixture of 3-[(3-fluoro-2-methoxyphenyl)amino]-2-{3-[(3R)-morpholin-3-ylmethoxy]pyridin-4-yl}-1H,5H,6H,7H-pyrrolo[3,2-c]pyridin-4-one (100 mg, 0.21 mmol, 1.00 equiv) in NMP (3.00 mL) was added (2E)-4-(dimethylamino)but-2-enoyl chloride (95 mg, 0.63 mmol, 3.00 equiv) in portions at 0 degrees C. under argon atmosphere. The resulting mixture was stirred for 1 h at 0 degrees C. under argon atmosphere. Desired product could be detected by LCMS. The reaction was quenched by the addition of MeOH (3 mL) at 0 degrees C. The resulting mixture was concentrated under reduced pressure. The crude product (80 mg) was purified by Prep-HPLC with the following conditions (Column: Xselect CSH F-Phenyl OBD column, 19*250 mm, 5 μm; Mobile Phase A: Water (10 mmol/L NH₄HCO₃), Mobile Phase B: ACN; Flow rate: 25 mL/min; Gradient: 55% B to 58% B in 11 min, 58% B; Wave Length: 254 nm; RT1(min): 9.85; Number Of Runs: 0) to afford 2-(3-{[(3R)-4-[(2E)-4-(dimethylamino)but-2-enoyl]morpholin-3-yl]methoxy}pyridin-4-yl)-3-[(3-fluoro-2-methoxyphenyl)amino]-1H,5H,6H,7H-pyrrolo[3,2-c]pyridin-4-one) (7.6 mg, 6.12%) as a white solid.

LC-MS: (M+H)⁺ found 579.40.

¹H NMR (400 MHz, DMSO-d6) δ 10.93 (s, 1H), 8.45 (s, 1H), 8.11-7.90 (m, 1H), 7.53 (d, 1H), 7.41-7.25 (m, 1H), 7.14 (d, 1H), 6.80-6.30 (m, 4H), 5.98 (d, 1H), 5.10-4.65 (m, 1H), 4.57-4.16 (m, 2H), 4.17-3.82 (m, 6H), 3.71-3.52 (m, 2H), 3.52-3.37 (m, 3H), 3.01 (d, 3H), 2.93-2.71 (m, 1H), 2.05 (d, 6H).

### Example 85. 3-[(3-chloro-5-fluoro-2-methoxyphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (compound 213)

### 85.1. Synthesis of tert-butyl (2S)-2-{[(4-{3-[(3-chloro-5-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate

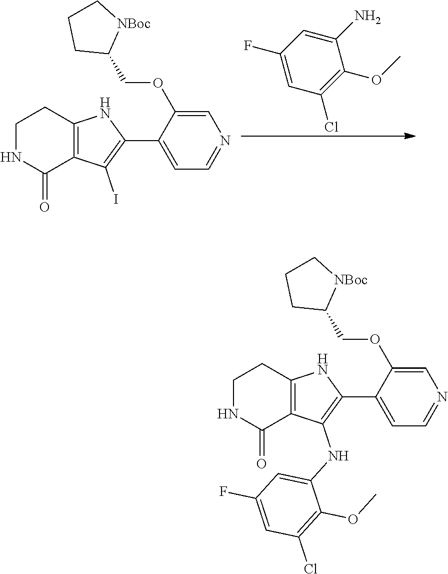 [SMILES: CC(C)(C)OC(=O)N1CCC[C@H]1COc1cnccc1-c1[nH]c2c(c1I)C(=O)NCC2 ; COc1c(N)cc(F)cc1Cl ; COc1c(Cl)cc(F)cc1Nc1c(-c2ccncc2OC[C@@H]2CCCN2C(=O)OC(C)(C)C)[nH]c2c1C(=O)NCC2]

To a stirred mixture of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (90 mg, 0.16 mmol, 1 equiv) and 3-chloro-5-fluoro-2-methoxyaniline (29 mg, 0.16 mmol, 1.0 equiv) in DMF (1.5 mL) was added EPhos Pd G4 (15 mg, 0.017 mmol, 0.1 equiv) and Cs₂CO₃ (163 mg, 0.50 mmol, 3 equiv) in portions at room temperature under argon atmosphere. The resulting mixture was stirred for 2 h at 50° C. under argon atmosphere. The reaction was monitored by LCMS. The resulting mixture was concentrated under vacuum. The residue was purified by silica gel column chromatography, eluted with CH₂Cl₂/MeOH (10:1) to afford tert-butyl (2S)-2-{[(4-{3-[(3-chloro-5-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (90 mg, 91.87%) as a yellow solid.

LC-MS: (M+H)⁺ found 586.0

### 85.2. Synthesis of (S)-3-((3-chloro-5-fluoro-2-methoxyphenyl)amino)-2-(3-(pyrrolidin-2-ylmethoxy)pyridin-4-yl)-1,5,6,7-tetrahydro-4H-pyrrolo[3,2-c]pyridin-4-one

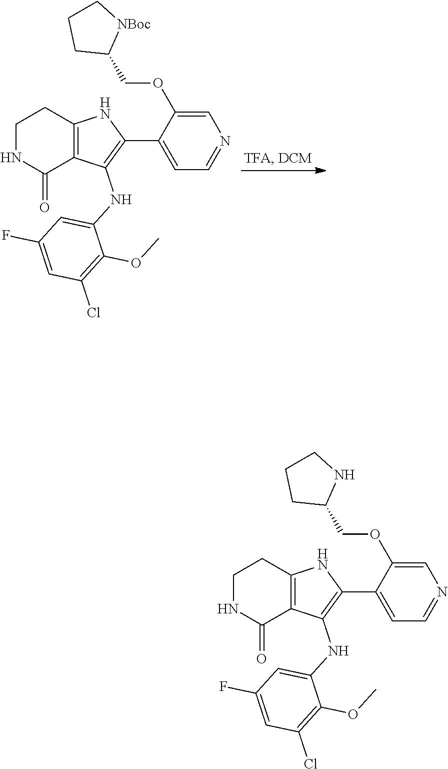 [SMILES: COc1c(Cl)cc(F)cc1Nc1c(-c2ccncc2OC[C@@H]2CCCN2C(=O)OC(C)(C)C)[nH]c2c1C(=O)NCC2 ; COc1c(Cl)cc(F)cc1Nc1c(-c2ccncc2OC[C@@H]2CCCN2)[nH]c2c1C(=O)NCC2]

To a stirred solution of tert-butyl (2S)-2-{[(4-{3-[(3-chloro-5-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (90 mg, 0.15 mmol, 1 equiv) in DCM (1.5 mL) was added TFA (0.3 mL) dropwise at 0° C. under air atmosphere. The reaction was monitored by LCMS. The resulting mixture was concentrated under reduced pressure. The crude product was used in the next step directly without further purification.

LC-MS: (M+H)⁺ found 486.2

### 85.3. Synthesis of 3-[(3-chloro-5-fluoro-2-methoxyphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one

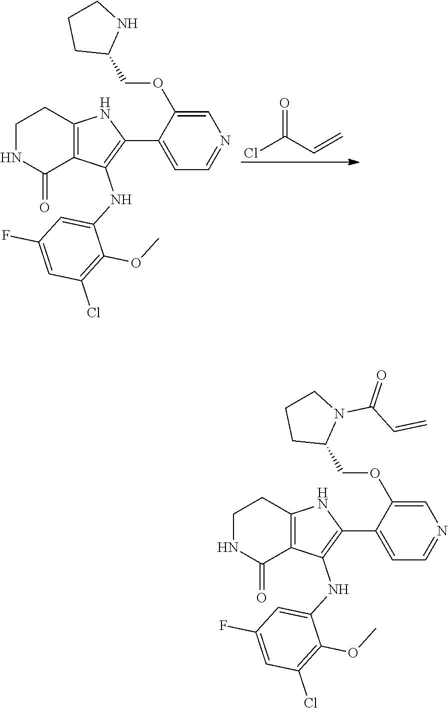

To a stirred solution of 3-[(3-chloro-5-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (crude) in THF and sat. NaHCO₃(aq.) (3 mL) was added acryloyl chloride (27 mg, 0.30 mmol, 2 equiv) dropwise at 0° C. under argon atmosphere. The reaction was monitored by LCMS. The resulting mixture was concentrated under vacuum. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 26% B to 54% B in 8 min, 54% B; Wave Length: 254/220 nm; RT1(min): 7.53; Number Of Runs: 0) to afford 3-[(3-chloro-5-fluoro-2-methoxyphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (6.5 mg, 7.79%) as a light yellow solid.

LC-MS: (M+H)⁺ found 540.0

¹H NMR (400 MHz, Chloroform-d) δ 11.55 (s, 1H), 8.24 (s, 1H), 8.02 (d, 1H), 7.58-7.40 (m, 2H), 7.25 (s, 1H), 6.63-6.48 (m, 1H), 6.46-6.28 (m, 2H), 6.05-5.96 (m, 1H), 5.82-5.70 (m, 1H), 5.27 (s, 1H), 5.03 (s, 1H), 4.25-4.10 (m, 1H), 4.17-3.81 (m, 4H), 3.90-3.66 (m, 2H), 3.65-3.55 (m, 2H), 3.29-3.15 (m, 2H), 2.30-2.04 (m, 3H), 1.87 (s, 2H).

### Example 86. 3-[(3-chloro-2-methoxyphenyl)amino]-2-(3-{[(2R,5R)-5-methyl-1-(prop-2-enoyl)pyrrolidin-2-yl]methoxy}pyridin-4-yl)-1H,5H,6H,7H-pyrrolo[3,2-c]pyridin-4-one (compound 267)

### 86.1. Synthesis of tert-butyl (2R,5R)-2-(hydroxymethyl)-5-methylpyrrolidine-1-carboxylate

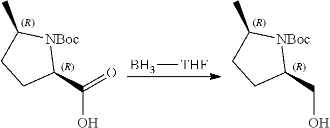

To a stirred solution of (2R,5R)-1-(tert-butoxycarbonyl)-5-methylpyrrolidine-2-carboxylic acid (1.0 g, 4.36 mmol, 1 equiv) in THF (10 mL) was added BH₃-THF (6.50 mL, 67.92 mmol, 15.57 equiv) dropwise at 0° C. under argon atmosphere. The resulting mixture was stirred for 1 h at 60° C. under argon atmosphere. Desired product could be detected by TLC. The mixture was allowed to cool down to room temperature. The reaction was quenched by the addition of MeOH (5 mL) at 0° C. The resulting mixture was concentrated under reduced pressure. The residue was purified by silica gel column chromatography, eluted with PE/EA (2:1) to afford tert-butyl (2R,5R)-2-(hydroxymethyl)-5-methylpyrrolidine-1-carboxylate (900 mg, 95.85%) as a yellow oil. Desired product could be detected by TLC.

### 86.2. Synthesis of tert-butyl (2R,5R)-2-{[(4-bromopyridin-3-yl)oxy]methyl}-5-methylpyrrolidine-1-carboxylate

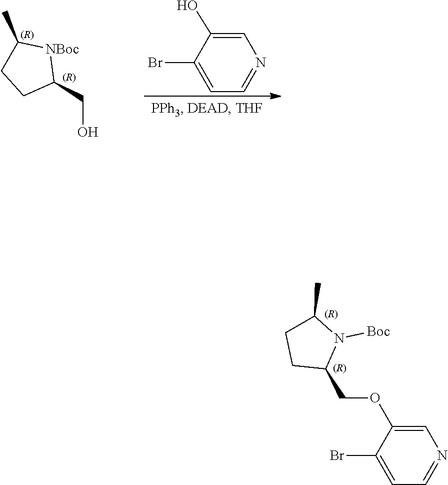

To a stirred solution of tert-butyl (2R,5R)-2-(hydroxymethyl)-5-methylpyrrolidine-1-carboxylate (890.88 mg, 4.138 mmol, 1.2 equiv) and 4-bromopyridin-3-ol (600 mg, 3.44 mmol, 1.00 equiv) in THF was added DEAD (900 mg, 5.17 mmol, 1.5 equiv) dropwise at 0° C. under argon atmosphere. The resulting mixture was stirred for 4h at room temperature under argon atmosphere. The reaction was monitored by LCMS. The resulting mixture was concentrated under reduced pressure. The residue was purified by silica gel column chromatography, eluted with CH₂Cl₂/MeOH (10:1) to afford tert-butyl (2R,5R)-2-{[(4-bromopyridin-3-yl)oxy]methyl}-5-methylpyrrolidine-1-carboxylate (1.2 g, 93.73%) as a colorless oil.

LC-MS: (M+H)⁺ found 372.90

### 86.3. Synthesis of tert-butyl (2R,5R)-2-methyl-5-{[(4-{4-oxo-1H,5H,6H,7H-pyrrolo[3,2-c]pyridin-2-yl}pyridin-3-yl)oxy]methyl}pyrrolidine-1-carboxylate

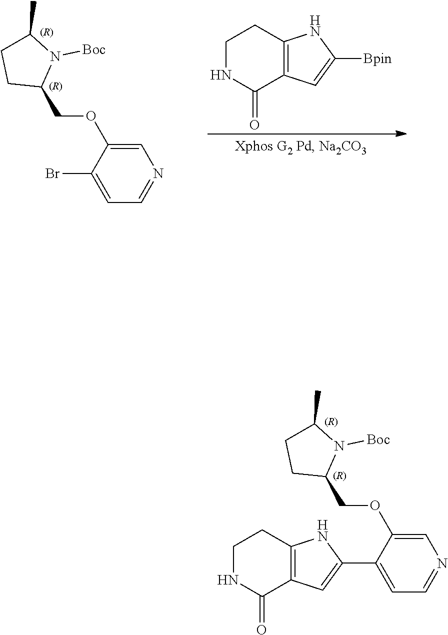

To a stirred mixture of tert-butyl (2R,5R)-2-{[(4-bromopyridin-3-yl)oxy]methyl}-5-methylpyrrolidine-1-carboxylate (1 g, 2.69 mmol, 1 equiv) and 2-(4,4,5,5-tetramethyl-1,3,2-dioxaborolan-2-yl)-1H,5H,6H,7H-pyrrolo[3,2-c]pyridin-4-one (0.85 g, 3.23 mmol, 1.2 equiv) in THF/H₂O was added Xphos Pd G2 (0.21 g, 0.27 mmol, 0.1 equiv) at room temperature under argon atmosphere. The resulting mixture was stirred for overnight at 50° C. under argon atmosphere. The reaction was monitored by LCMS. The resulting mixture was extracted with EtOAc (5×30 mL). The combined organic layers were washed with water (3×10 mL), dried over anhydrous Na2SO4. After filtration, the filtrate was concentrated under reduced pressure. The residue was purified by silica gel column chromatography, eluted with CH₂Cl₂/MeOH (10:1) to afford tert-butyl (2R,5R)-2-methyl-5-{[(4-{4-oxo-1H,5H,6H,7H-pyrrolo[3,2-c]pyridin-2-yl}pyridin-3-yl)oxy]methyl}pyrrolidine-1-carboxylate (730 mg, 63.55%) as a light yellow solid.

LC-MS: (M+H)⁺ found 427.3.

### 86.4. Synthesis of tert-butyl (2R,5R)-2-{[(4-{3-iodo-4-oxo-1H,5H,6H,7H-pyrrolo[3,2-c]pyridin-2-yl}pyridin-3-yl)oxy]methyl}-5-methylpyrrolidine-1-carboxylate

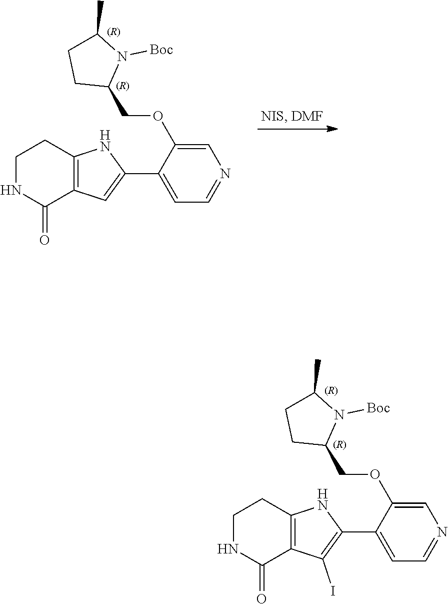

To a stirred mixture of tert-butyl (2R,5R)-2-methyl-5-{[(4-{4-oxo-1H,5H,6H,7H-pyrrolo[3,2-c]pyridin-2-yl}pyridin-3-yl)oxy]methyl}pyrrolidine-1-carboxylate (300 mg, 1 equiv) in DMF was added NIS (189 mg, 0.84 mmol, 1.2 equiv) in portions at 0° C. under argon atmosphere. The resulting mixture was stirred for 1 h at room temperature under argon atmosphere. The reaction was monitored by LCMS. The reaction was quenched by the addition of sat. sodium sulfite (aq.) (50 mL) at 0° C. The precipitated solids were collected by filtration and washed with water (3×10 mL). The resulting mixture was concentrated under vacuum. This resulted in tert-butyl (2R,5R)-2-{[(4-{3-iodo-4-oxo-1H,5H,6H,7H-pyrrolo[3,2-c]pyridin-2-yl}pyridin-3-yl)oxy]methyl}-5-methylpyrrolidine-1-carboxylate (700 mg, 74.04%) as a light yellow solid.

LC-MS: (M+H)⁺ found 552.95.

### 86.5. Synthesis of tert-butyl (2R,5R)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-5-methylpyrrolidine-1-carboxylate

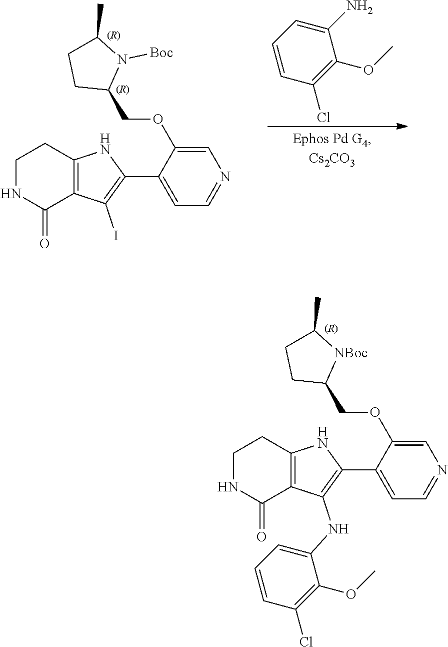

To a stirred mixture of tert-butyl (2R,5R)-2-{[(4-{3-iodo-4-oxo-1H,5H,6H,7H-pyrrolo[3,2-c]pyridin-2-yl}pyridin-3-yl)oxy]methyl}-5-methylpyrrolidine-1-carboxylate (300 mg, 0.54 mmol, 1 equiv) and 3-chloro-2-methoxyaniline (85 mg, 0.54 mmol, 1 equiv) in DMF was added EPhos Pd G4 (49 mg, 0.054 mmol, 0.1 equiv) and Cs₂CO₃ (530 mg, 1.62 mmol, 3 equiv) at room temperature under argon atmosphere. The reaction was monitored by LCMS. The resulting mixture was concentrated under vacuum. The residue was purified by silica gel column chromatography, eluted with CH₂Cl₂/MeOH (10:1) to afford tert-butyl (2R,5R)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-5-methylpyrrolidine-1-carboxylate (300 mg, 94.90%) as a light yellow solid.

LC-MS: (M+H)⁺ found 582.25.

### 86.6. Synthesis of 3-((3-chloro-2-methoxyphenyl)amino)-2-(3-(((2R,5R)-5-methylpyrrolidin-2-yl)methoxy)pyridin-4-yl)-1,5,6,7-tetrahydro-4H-pyrrolo[3,2-c]pyridin-4-one

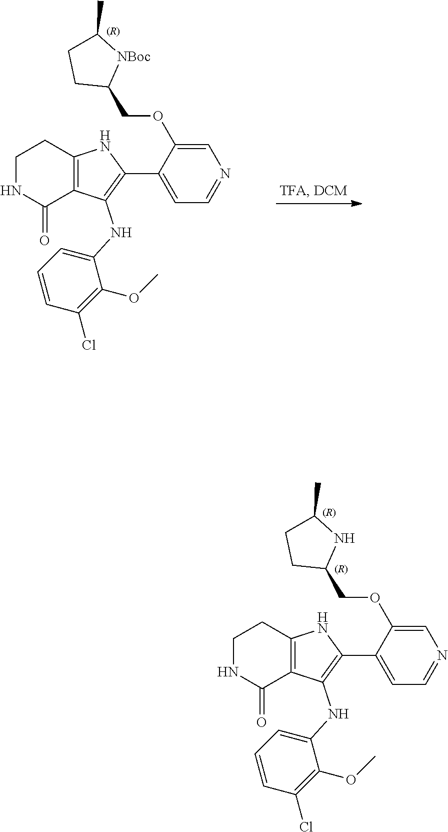

To a stirred solution of tert-butyl (2R,5R)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-5-methylpyrrolidine-1-carboxylate (150 mg, 0.25 mmol, 1 equiv) in DCM was added TFA (0.5 mL) dropwise at 0° C. under argon atmosphere. The reaction was monitored by LCMS. The resulting mixture was concentrated under reduced pressure. The resulting mixture was used in the next step directly without further purification.

LC-MS: (M+H)⁺ found 482.25.

### 86.7. Synthesis of 3-[(3-chloro-2-methoxyphenyl)amino]-2-(3-{[(2R,5R)-5-methyl-1-(prop-2-enoyl)pyrrolidin-2-yl]methoxy}pyridin-4-yl)-1H,5H,6H,7H-pyrrolo[3,2-c]pyridin-4-one

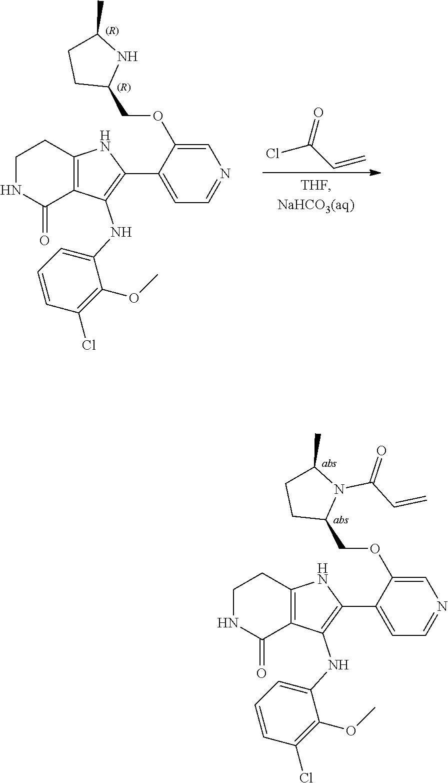

To a stirred solution of 3-[(3-chloro-2-methoxyphenyl)amino]-2-(3-{[(2R,5R)-5-methylpyrrolidin-2-yl]methoxy}pyridin-4-yl)-1H,5H,6H,7H-pyrrolo[3,2-c]pyridin-4-one (125 mg, 0.25 mmol, 1 equiv) in THF and sat. NaHCO₃(aq.) (3 mL) was added dropwise acryloyl chloride (23 mg, 0.25 mmol, 1 equiv) at 0° C. under argon atmosphere. The resulting mixture was concentrated under vacuum. The crude product (150 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 36% B to 66% B in 7 min; Wave Length: 254 nm; RT1(min): 6.5; Number Of Runs: 0) to afford 3-[(3-chloro-2-methoxyphenyl)amino]-2-(3-f{[(2R,5R)-5-methyl-1-(prop-2-enoyl)pyrrolidin-2-yl]methoxy}pyridin-4-yl)-1H,5H,6H,7H-pyrrolo[3,2-c]pyridin-4-one (29.4 mg, 21.06%) as a light yellow solid.

LC-MS: (M+H)⁺ found 536.1

¹H NMR (400 MHz, Chloroform-d) δ 11.67 (s, 1H), 8.24 (s, 1H), 8.00 (d, 1H), 7.59 (s, 1H), 7.51 (d, 1H), 6.77-6.71 (m, 1H), 6.69-6.54 (m, 2H), 6.47-6.41 (m, 1H), 6.27-6.21 (m, 1H), 5.85-5.80 (m, 1H), 5.26 (s, 1H), 5.07-4.94 (m, 1H), 4.35-4.20 (m, 3H), 4.11 (s, 3H), 3.69-3.57 (m, 2H), 3.23 (m, 2H), 2.38-2.18 (m, 2H), 1.98-1.90 (m, 2H), 1.44 (d, 3H).

### Example 87. 3-[(2-methoxy-3-methylphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (compound 277)

### 87.1. Synthesis of tert-butyl (2S)-2-{[(4-{3-[(2-methoxy-3-methylphenyl)amino]-4-oxo-1H,5H,6H,7H-pyrrolo[3,2-c]pyridin-2-yl}pyridin-3-yl)oxy]methyl}pyrrolidine-1-carboxylate

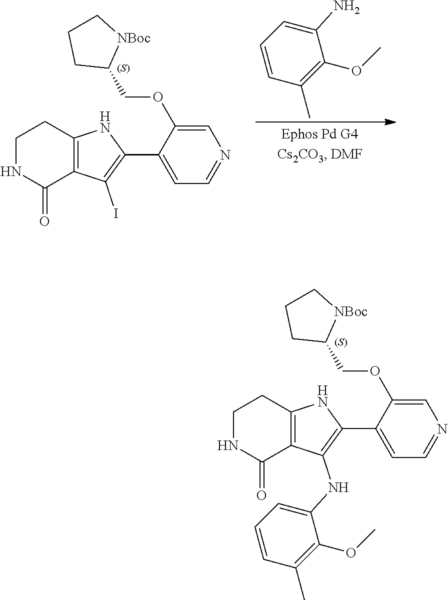

To a stirred mixture of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (100 mg, 0.18 mmol, 1 equiv) and 2-methoxy-3-methylaniline (50 mg, 0.37 mmol, 2 equiv) in DMF (1 mL) were added Cs₂CO₃ (121 mg, 0.37 mmol, 2 equiv) and Ephos Pd G4 (17 mg, 0.019 mmol, 0.1 equiv) in portions at room temperature under argon atmosphere. The resulting mixture was stirred for 2 h at 50° C. under argon atmosphere. Desired product could be detected by LCMS. The resulting mixture was concentrated under vacuum. The residue was purified by silica gel column chromatography, eluted with CH₂Cl₂/MeOH (15:1) to afford tert-butyl (2S)-2-{[(4-{3-[(2-methoxy-3-methylphenyl)amino]-4-oxo-1H,5H,6H,7H-pyrrolo[3,2-c]pyridin-2-yl}pyridin-3-yl)oxy]methyl}pyrrolidine-1-carboxylate (80 mg, 78.65%) as a yellow solid.

LC-MS: [M+H]− found 548.00.

### 87.2. Synthesis of 3-[(2-methoxy-3-methylphenyl)amino]-2-{3-[(2S)-pyrrolidin-2-ylmethoxy]pyridin-4-yl}-1H,5H,6H,7H-pyrrolo[3,2-c]pyridin-4-one

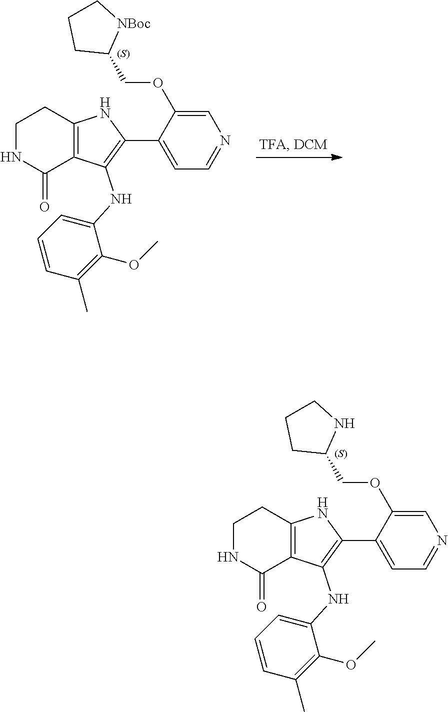

A mixture of tert-butyl (2S)-2-{[(4-{3-[(2-methoxy-3-methylphenyl)amino]-4-oxo-1H,5H,6H,7H-pyrrolo[3,2-c]pyridin-2-yl}pyridin-3-yl)oxy]methyl}pyrrolidine-1-carboxylate (80 mg, 0.14 mmol, 1 equiv) in TFA (1 mL) and DCM (3 mL) was stirred for 1 h at room temperature under air atmosphere. Desired product could be detected by LCMS. The resulting mixture was concentrated under vacuum. This resulted in 3-[(2-methoxy-3-methylphenyl)amino]-2-{3-[(2S)-pyrrolidin-2-ylmethoxy]pyridin-4-yl}-1H,5H,6H,7H-pyrrolo[3,2-c]pyridin-4-one (65 mg, 99.43%) as a yellow oil.

LC-MS: [M+H]⁺ found 448.00.

### 87.3. Synthesis of 3-[(2-methoxy-3-methylphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one

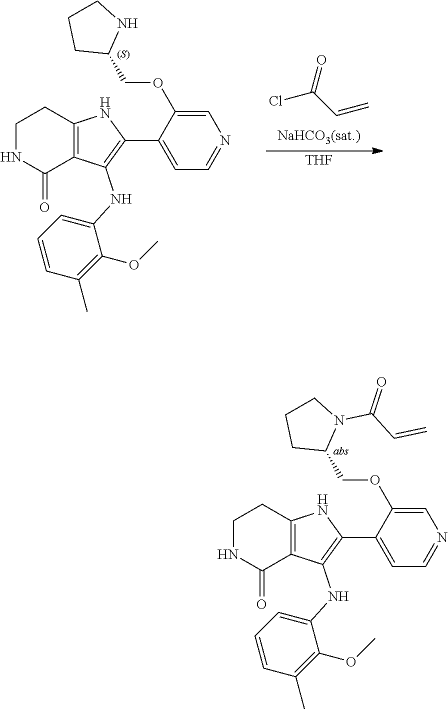

A mixture of 3-[(2-methoxy-3-methylphenyl)amino]-2-{3-[(2S)-pyrrolidin-2-ylmethoxy]pyridin-4-yl}-1H,5H,6H,7H-pyrrolo[3,2-c]pyridin-4-one (80 mg, 0.17 mmol, 1 equiv) and acryloyl chloride (17 mg, 0.19 mmol, 1.1 equiv) in THF (1 mL) and sat. NaHCO₃(aq.) (1 mL) was stirred for 1 h at 0° C. under air atmosphere. Desired product could be detected by LCMS. The resulting mixture was concentrated under reduced pressure. The crude product was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 24% B to 45% B in 9 min, 45% B; Wave Length: 254/220 nm; RT1(min): 8.85; Number Of Runs: 0) to afford 3-[(2-methoxy-3-methylphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (9.8 mg, 10.83%) as a light yellow solid.

LC-MS: [M+H]+ found 502.00.

¹H NMR (300 MHz, Chloroform-d) δ 11.44 (s, 1H), 8.20 (s, 1H), 7.94 (d, J=4.8 Hz, 1H), 7.65 (s, 1H), 7.48 (d, J=5.4 Hz, 1H), 6.63-6.50 (m, 3H), 6.42-6.36 (m, 1H), 6.17-6.14 (m, 1H), 5.79-5.75 (m, 1H), 5.20 (s, 1H), 5.04 (s, 1H), 4.26 (t, J=9.6 Hz, 1H), 4.12-4.08 (m, 1H), 3.97 (s, 3H), 3.75 (t, J=6.5 Hz, 2H), 3.66-3.53 (m, 2H), 3.21 (t, J=6.5 Hz, 2H), 2.32 (s, 3H), 2.16 (s, 3H), 1.87 (s, 1H).

### Example 88. 2-(3-{[(2R)-1-(but-2-ynoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (compound 289)

### 88.1. Synthesis of tert-butyl (2R)-2-{[(4-bromopyridin-3-yl)oxy]methyl}azetidine-1-carboxylate

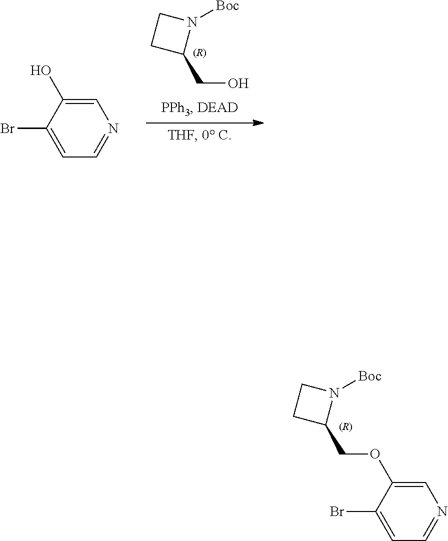

To a stirred solution/mixture of 4-bromopyridin-3-ol (5 g, 28.73 mmol, 1 equiv) and tert-butyl (2R)-2-(hydroxymethyl)azetidine-1-carboxylate (6.44 mg, 34.47 mmol, 1.2 equiv) in 8 mL THF was added PPh₃ (11.31 g, 43.10 mmol, 1.5 equiv) in portions at 0° C. under nitrogen atmosphere. Stirred for 30 min at the same temperature and DEAD (7.51 g, 43.10 mmol, 1.5 equiv) was added, stirred for overnight. The resulting mixture was concentrated under reduced pressure. The residue was purified by reverse flash chromatography with the following conditions: column, C18 silica gel; mobile phase, ACN in water, 10% to 100% gradient in 30 min; detector, UV 254 nm. Tert-butyl (2R)-2-{[(4-bromopyridin-3-yl)oxy]methyl}azetidine-1-carboxylate (7.5 g, 76.04%) was obtained as light grey oil.

LC-MS: (M+H)⁺ found: 344.85

### 88.2. Synthesis of tert-butyl (2R)-2-{[(4-{4-oxo-1H,5H,6H,7H-pyrrolo[3,2-c]pyridin-2-yl}pyridin-3-yl)oxy]methyl}azetidine-1-carboxylate

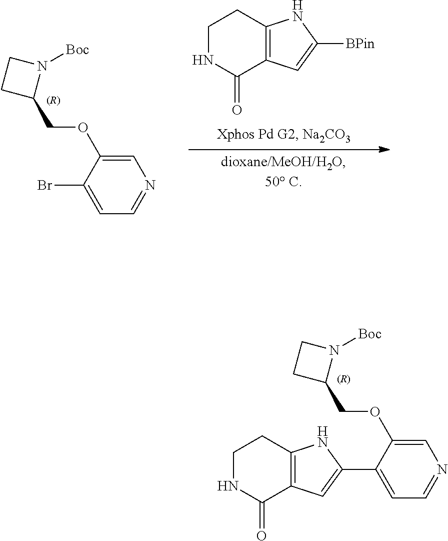

Into a 15 mL sealed tube were added tert-butyl (2R)-2-{[(4-bromopyridin-3-yl)oxy]methyl}azetidine-1-carboxylate (500 mg, 1.45 mmol, 1 equiv), 2-(4,4,5,5-tetramethyl-1,3,2-dioxaborolan-2-yl)-1,5,6,7-tetrahydro-4H-pyrrolo[3,2-c]pyridin-4-one (216 mg, 1.60 mmol, 1.1 equiv), Na₂CO₃ (463 mg, 4.37 mmol, 3.0 equiv), XPhos palladium(II) biphenyl-2-amine chloride (114 mg, 0.14 mmol, 0.1 equiv), dioxane (5 mL), MeOH (1.5 mL) and H₂O (1 mL) at rt, stirred at 50° C. for 2.5 h under Ar atmosphere. The resulting mixture was concentrated under reduced pressure. The residue was purified by reverse flash chromatography with the following conditions: column, C18 silica gel; mobile phase, ACN in water, 0% to 100% gradient in 30 min; detector, UV 254 nm. This resulted in tert-butyl (2R)-2-{[(4-{4-oxo-1H,5H,6H,7H-pyrrolo[3,2-c]pyridin-2-yl}pyridin-3-yl)oxy]methyl}azetidine-1-carboxylate (400 mg, 68.91%) as a light brown solid.

LC-MS: (M+H)⁺ found: 399.10

### 88.3. Synthesis of tert-butyl (2R)-2-{[(4-{3-iodo-4-oxo-1H,5H,6H,7H-pyrrolo[3,2-c]pyridin-2-yl}pyridin-3-yl)oxy]methyl}azetidine-1-carboxylate

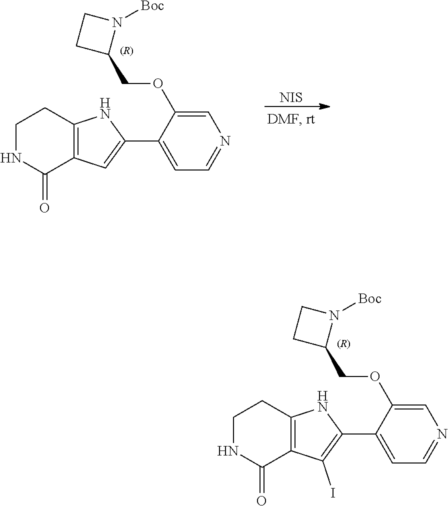

To a stirred solution of tert-butyl (2R)-2-{[(4-{4-oxo-1H,5H,6H,7H-pyrrolo[3,2-c]pyridin-2-yl}pyridin-3-yl)oxy]methyl}azetidine-1-carboxylate (5.5 g, 13.80 mmol, 1 equiv) in DMF (15 mL) was added NIS (3.42 g, 15.18 mmol, 1.1 equiv) in portions at room temperature. Stirred for 1 h, then was purified by reverse flash chromatography with the following conditions: column, C18 silica gel; mobile phase, ACN in water, 0% to 100% gradient in 30 min; detector, UV 254 nm to give tert-butyl (2R)-2-{[(4-{3-iodo-4-oxo-1H,5H,6H,7H-pyrrolo[3,2-c]pyridin-2-yl}pyridin-3-yl)oxy]methyl}azetidine-1-carboxylate (6.1 g, 84.28%) as brown yellow solid. LC-MS: (M+H)⁺ found: 525.15

### 88.4. Synthesis of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate

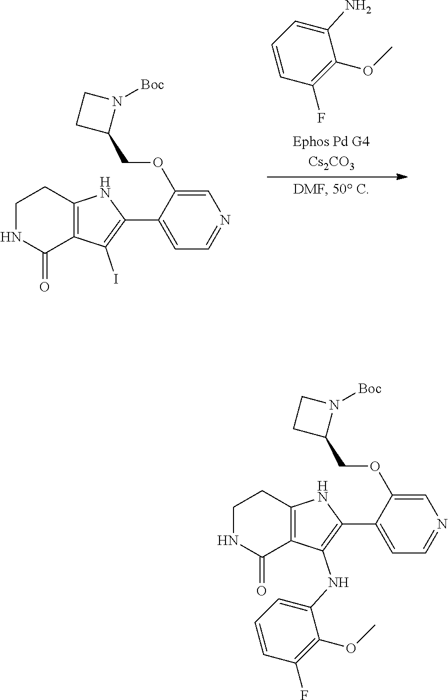

Into a 10 mL sealed tube were added tert-butyl (2R)-2-{[(4-{3-iodo-4-oxo-1H,5H,6H,7H-pyrrolo[3,2-c]pyridin-2-yl}pyridin-3-yl)oxy]methyl}azetidine-1-carboxylate (500 mg, 0.95 mmol, 1 equiv), 3-fluoro-2-methoxyaniline (201 mg, 1.43 mmol, 1.5 equiv), Cs₂CO₃ (932 mg, 2.86 mmol, 3.0 equiv), EPhos Pd G4 (87 mg, 0.095 mmol, 0.1 equiv) and DMF (2 mL) at room temperature, then stirred for 2 h under Ar atmosphere. The resulting mixture was concentrated under reduced pressure. The residue was purified by silica gel column chromatography, eluted with CH₂Cl₂/MeOH (9:1) to afford tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (505 mg, 98.51%) as a brown yellow solid.

LC-MS: (M+H)⁺ found: 538.15.

### 88.5. Synthesis of 2-{3-[(2R)-azetidin-2-ylmethoxy]pyridin-4-yl}-3-[(3-fluoro-2-methoxyphenyl)amino]-1H,5H,6H,7H-pyrrolo[3,2-c]pyridin-4-one

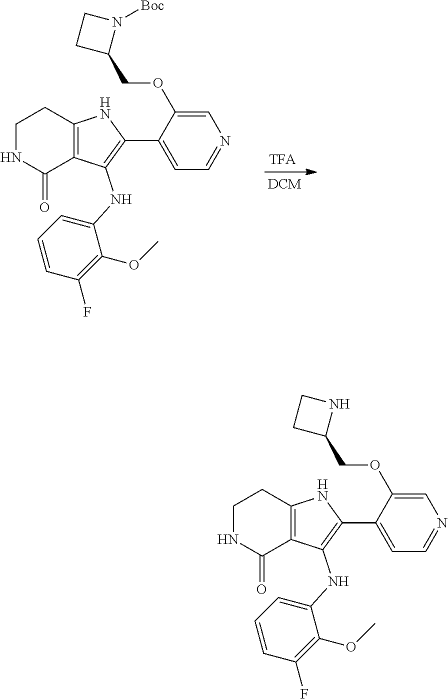 [SMILES: COc1c(F)cccc1Nc1c(-c2ccncc2OC[C@H]2CCN2C(=O)OC(C)(C)C)[nH]c2c1C(=O)NCC2 ; COc1c(F)cccc1Nc1c(-c2ccncc2OC[C@H]2CCN2)[nH]c2c1C(=O)NCC2]

Into a 50 mL round-bottom flask were added tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (400 mg, 0.74 mmol, 1 equiv), then trifluoroacetaldehyde (10 mL) and DCM (30 mL) was added at 0° C. Stirred for 2 h at room temperature. The resulting mixture was concentrated under reduced pressure to afford 2-{3-[(2R)-azetidin-2-ylmethoxy]pyridin-4-yl}-3-[(3-fluoro-2-methoxyphenyl)amino]-1H,5H,6H,7H-pyrrolo[3,2-c]pyridin-4-one (320 mg, 98.31%) as a brown yellow solid.

LC-MS: (M+H)⁺ found: 438.05

### 88.6. Synthesis of 2-(3-{[(2R)-1-(but-2-ynoyl)azetidin-2-yl]methoxy}pyridin-4-yl)-3-[(3-fluoro-2-methoxyphenyl)amino]-1H,5H,6H,7H-pyrrolo[3,2-c]pyridin-4-one

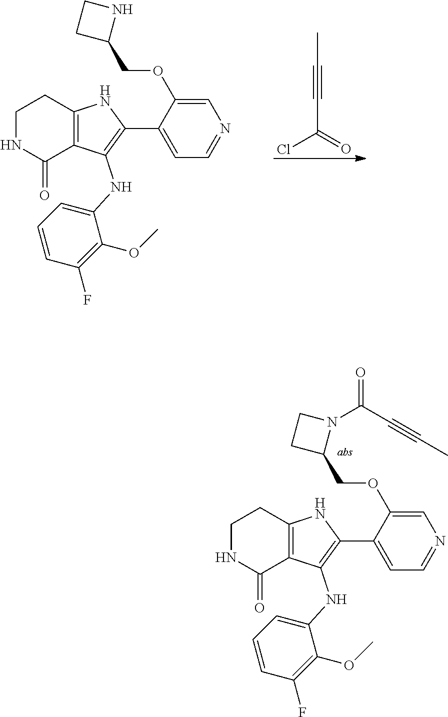

To a stirred solution of 2-{3-[(2R)-azetidin-2-ylmethoxy]pyridin-4-yl}-3-[(3-fluoro-2-methoxyphenyl)amino]-1H,5H,6H,7H-pyrrolo[3,2-c]pyridin-4-one (330 mg, 0.75 mmol, 1 equiv) and 2-butynoic acid (190 mg, 2.26 mmol, 3 equiv) in tetrahydrofuran (10 mL) was added T₃P (480 mg, 1.50 mmol, 2.0 equiv) at room temperature. Stirred for 2 h at rt. The resulting mixture was extracted with EtOAc (2×50 mL), dried over anhydrous Na₂SO₄. After filtration, the filtrate was concentrated under reduced pressure. The crude product (500 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 58% B in 7 min; Wave Length: 254 nm; RT1(min): 6.5; Number Of Runs: 0) to afford 2-(3-{[(2R)-1-(but-2-ynoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (150 mg, 39.49%) as a brown yellow solid.

LC-MS: (M+H)⁺ found: 504.10

¹H NMR (300 MHz, Chloroform-d) δ 11.37 (s, 1H), 8.24 (s, 1H), 8.00 (s, 1H), 7.52 (d, J=6.8 Hz, 2H), 6.57 (td, J=8.2, 5.9 Hz, 1H), 6.45 (ddd, J=10.9, 8.3, 1.5 Hz, 1H), 6.07 (d, J=8.2 Hz, 1H), 5.21 (s, 1H), 4.94 (q, J=8.6 Hz, 1H), 4.49 (t, J=9.8 Hz, 1H), 4.25 (ddd, J=10.9, 5.7, 2.4 Hz, 3H), 4.10 (d, J=1.2 Hz, 3H), 3.57 (td, J=6.8, 2.5 Hz, 2H), 3.25-2.95 (m, 2H), 2.71-2.53 (m, 1H), 2.20-2.06 (m, 1H), 2.03 (s, 3H).

### Example 89. 3-[(2-ethyl-3-fluorophenyl)amino]-2-(3-{[(2R)-1-(prop-2-enoyl)pyrrolidin-2-yl]methoxy}pyridin-4-yl)-1H,5H,6H,7H-pyrrolo[3,2-c]pyridin-4-one (compound 294)

### 89.1. Synthesis of tert-butyl (2R)-2-{[(4-{3-[(2-ethyl-3-fluorophenyl)amino]-4-oxo-1H,5H,6H,7H-pyrrolo[3,2-c]pyridin-2-yl}pyridin-3-yl)oxy]methyl}pyrrolidine-1-carboxylate

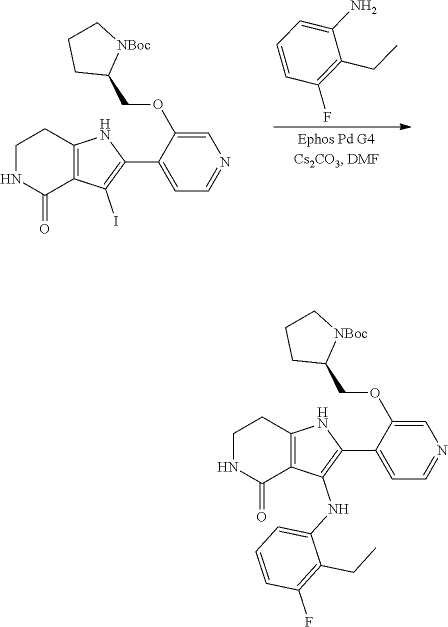

To a stirred mixture of tert-butyl (2R)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (200 mg, 0.37 mmol, 1 equiv) and 2-ethyl-3-fluoroaniline (62 mg, 0.44 mmol, 1.2 equiv) in DMF (2 mL) were added Cs₂CO₃ (242 mg, 0.74 mmol, 2 equiv) and Ephos Pd G4 (34 mg, 0.037 mmol, 0.1 equiv) in portions at room temperature under argon atmosphere. The resulting mixture was stirred for 2 h at 50° C. under argon atmosphere. Desired product could be detected by LCMS. The resulting mixture was concentrated under reduced pressure. The residue was purified by silica gel column chromatography, eluted with CH₂Cl₂/MeOH (20:1) to afford tert-butyl (2R)-2-{[(4-{3-[(2-ethyl-3-fluorophenyl)amino]-4-oxo-1H,5H,6H,7H-pyrrolo[3,2-c]pyridin-2-yl}pyridin-3-yl)oxy]methyl}pyrrolidine-1-carboxylate (182 mg, 89.14%) as a yellow solid.

LC-MS: [M+H]− found 550.00.

### 89.2. Synthesis of 3-[(2-ethyl-3-fluorophenyl)amino]-2-{3-[(2R)-pyrrolidin-2-ylmethoxy]pyridin-4-yl}-1H,5H,6H,7H-pyrrolo[3,2-c]pyridin-4-one

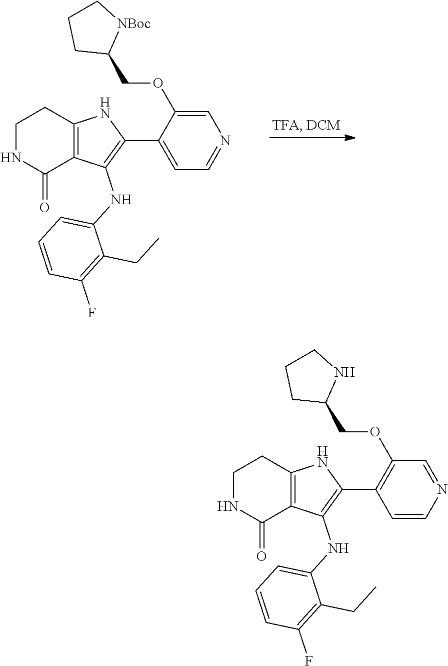 [SMILES: CCc1c(F)cccc1Nc1c(-c2ccncc2OC[C@H]2CCCN2C(=O)OC(C)(C)C)[nH]c2c1C(=O)NCC2 ; CCc1c(F)cccc1Nc1c(-c2ccncc2OC[C@H]2CCCN2)[nH]c2c1C(=O)NCC2]

A mixture of tert-butyl (2R)-2-{[(4-{3-[(2-ethyl-3-fluorophenyl)amino]-4-oxo-1H,5H,6H,7H-pyrrolo[3,2-c]pyridin-2-yl}pyridin-3-yl)oxy]methyl}pyrrolidine-1-carboxylate (170 mg, 0.30 mmol, 1 equiv) in TFA (3 mL) and DCM (3 mL) was stirred for 1 h at room temperature under air atmosphere. Desired product could be detected by LCMS. The resulting mixture was concentrated under reduced pressure. This resulted in 3-[(2-ethyl-3-fluorophenyl)amino]-2-{3-[(2R)-pyrrolidin-2-ylmethoxy]pyridin-4-yl}-1H,5H,6H,7H-pyrrolo[3,2-c]pyridin-4-one (130 mg, 93.50%) as a yellow oil.

LC-MS: [M+H]+ found 450.00.

### 89.3. Synthesis of 3-[(2-ethyl-3-fluorophenyl)amino]-2-(3-{[(2R)-1-(prop-2-enoyl)pyrrolidin-2-yl]methoxy}pyridin-4-yl)-1H,5H,6H,7H-pyrrolo[3,2-c]pyridin-4-one

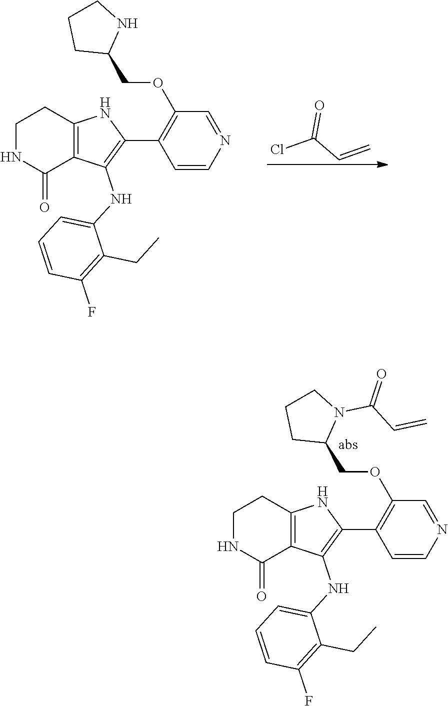

A mixture of 3-[(2-ethyl-3-fluorophenyl)amino]-2-{3-[(2R)-pyrrolidin-2-ylmethoxy]pyridin-4-yl}-1H,5H,6H,7H-pyrrolo[3,2-c]pyridin-4-one (110 mg, 0.24 mmol, 1 equiv) and acryloyl chloride (26 mg, 0.29 mmol, 1.2 equiv) in THF (2 mL) and sat. NaHCO₃(aq.) (2 mL) was stirred for 1 h at 0° C. under air atmosphere. Desired product could be detected by LCMS. The resulting mixture was concentrated under reduced pressure. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 26% B to 50% B in 9 min, 50% B; Wave Length: 254/220 nm; RT1(min): 8.85; Number Of Runs: 0) to afford 3-[(2-ethyl-3-fluorophenyl)amino]-2-(3-{[(2R)-1-(prop-2-enoyl)pyrrolidin-2-yl]methoxy}pyridin-4-yl)-1H,5H,6H,7H-pyrrolo[3,2-c]pyridin-4-one (10.5 mg, 8.50%) as a yellow solid.

LC-MS: [M+H]⁺ found 504.00.

¹H NMR (300 MHz, DMSO-d6) δ 11.50 (s, 1H), 8.39 (s, 1H), 7.91 (d, J=5.1 Hz, 1H), 7.36 (s, 1H), 7.24 (d, J=12.9 Hz, 1H), 7.15 (s, 1H), 6.78-6.62 (m, 2H), 6.48-6.32 (m, 1H), 6.27-6.21 (m, 1H), 6.07 (d, J=8.2 Hz, 1H), 5.77-5.73 (m, 1H), 4.68 (s, 1H), 4.32 (t, J=8.7 Hz, 1H), 4.17-4.13 (m, 1H), 3.65 (s, 2H), 3.45 (s, 2H), 2.99 (t, J=6.8 Hz, 2H), 2.70 (d, J=7.8 Hz, 2H), 2.09-1.87 (m, 4H), 1.25-1.16 (m, 3H).

### Example 90. 2-(3-{[(2R)-1-[(2E)-4-(dimethylamino)but-2-enoyl]pyrrolidin-2-yl]methoxy}pyridin-4-yl)-3-[(3-fluoro-2-methoxyphenyl)amino]-1H,5H,6H,7H-pyrrolo[3,2-c]pyridin-4-one (compound 298)

### 90.1. Synthesis of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate

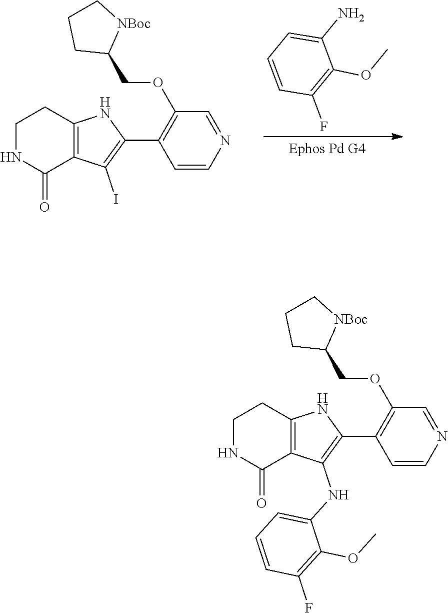

To a stirred mixture of tert-butyl (2R)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (200 mg, 0.37 mmol, 1 equiv) and 3-fluoro-2-methoxyaniline (104 mg, 0.74 mmol, 2 equiv) in DMF (2 mL) were added Ephos Pd G4 (34 mg, 0.037 mmol, 0.1 equiv) and Cs₂CO₃ (242 mg, 0.74 mmol, 2 equiv) in portions at room temperature under argon atmosphere. The resulting mixture was stirred for 1 h at 50° C. under argon atmosphere. Desired product could be detected by LCMS. The resulting mixture was concentrated under reduced pressure. The residue was purified by silica gel column chromatography, eluted with CH₂Cl₂/MeOH (20:1) to afford tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (160 mg, 78.08%) as a yellow solid.

LC-MS: [M+H]− found 552.00.

### 90.2. Synthesis of 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one

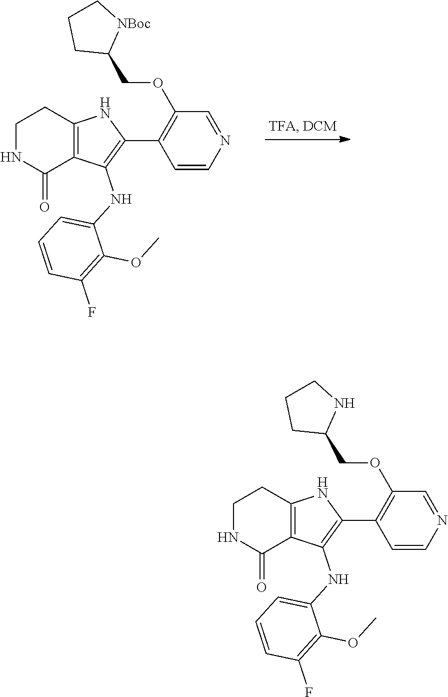 [SMILES: COc1c(F)cccc1Nc1c(-c2ccncc2OC[C@H]2CCCN2C(=O)OC(C)(C)C)[nH]c2c1C(=O)NCC2 ; COc1c(F)cccc1Nc1c(-c2ccncc2OC[C@H]2CCCN2)[nH]c2c1C(=O)NCC2]

A mixture of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (80 mg, 0.14 mmol, 1 equiv) in TFA (1 mL) and DCM (1 mL) was stirred for 1 h at room temperature under air atmosphere. Desired product could be detected by LCMS. The resulting mixture was concentrated under vacuum. The resulting mixture was extracted with CH₂Cl₂ (2×30 mL). The combined organic layers were washed with sat. NaHCO₃(aq.) (2×30 mL), dried over anhydrous Na₂SO₄. After filtration, the filtrate was concentrated under reduced pressure. This resulted in 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one (60 mg, 91.63%) as a yellow solid.

LC-MS: [M+H]+ found 452.00.

### 90.3. Synthesis of 2-(3-{[(2R)-1-[(2E)-4-(dimethylamino)but-2-enoyl]pyrrolidin-2-yl]methoxy}pyridin-4-yl)-3-[(3-fluoro-2-methoxyphenyl)amino]-1H,5H,6H,7H-pyrrolo[3,2-c]pyridin-4-one

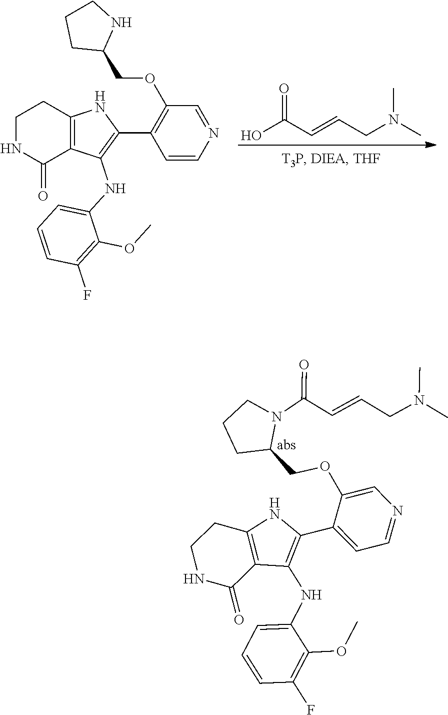

To a stirred mixture of 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one (60 mg, 0.13 mmol, 1 equiv) and (2E)-4-(dimethylamino)but-2-enoic acid (25 mg, 0.20 mmol, 1.5 equiv) in THF (2 mL) were added DIEA (171 mg, 1.33 mmol, 10 equiv) and T₃P (126 mg, 0.39 mmol, 3 equiv) in portions at 0° C. under air atmosphere. The resulting mixture was stirred for 1 h at room temperature under air atmosphere. Desired product could be detected by LCMS. The aqueous layer was extracted with CH₂Cl₂ (2×20 mL). The resulting mixture was concentrated under reduced pressure. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 18% B to 43% B in 9 min, 43% B; Wave Length: 254/220 nm; RT1(min): 8.85; Number Of Runs: 0) to afford 2-(3-{[(2R)-1-[(2E)-4-(dimethylamino)but-2-enoyl]pyrrolidin-2-yl]methoxy}pyridin-4-yl)-3-[(3-fluoro-2-methoxyphenyl)amino]-1H,5H,6H,7H-pyrrolo[3,2-c]pyridin-4-one (16.6 mg, 21.54%) as a light yellow solid.

LC-MS: [M+H]+ found 563.00.

¹H NMR (300 MHz, DMSO-d6) δ 11.53 (s, 1H), 8.41 (s, 1H), 7.98 (d, J=5.1 Hz, 1H), 7.46 (s, 1H), 7.36 (d, J=5.1 Hz, 1H), 7.13 (s, 1H), 6.80-6.54 (m, 2H), 6.52-6.38 (m, 2H), 5.98 (d, J=8.1 Hz, 1H), 4.67 (s, 1H), 4.38-4.32 (m, 1H), 4.18-4.13 (m, 1H), 3.91 (s, 3H), 3.63 (d, J=5.9 Hz, 2H), 3.42 (d, J=2.4 Hz, 3H), 3.08-2.92 (m, 3H), 2.13 (s, 5H), 2.08-1.84 (m, 5H)

### Example 91. 2-(3-{[(2R)-1-(but-2-ynoyl)azetidin-2-yl]methoxy}pyridin-4-yl)-3-{[3-(trifluoromethyl)phenyl]amino}-1H,5H,6H,7H-pyrrolo[3,2-c]pyridin-4-one (compound 349)

### 91.1. Synthesis of tert-butyl (2R)-2-({[4-(4-oxo-3-{[3-(trifluoromethyl)phenyl]amino}-1H,5H,6H,7H-pyrrolo[3,2-c]pyridin-2-yl)pyridin-3-yl]oxy}methyl)azetidine-1-carboxylate

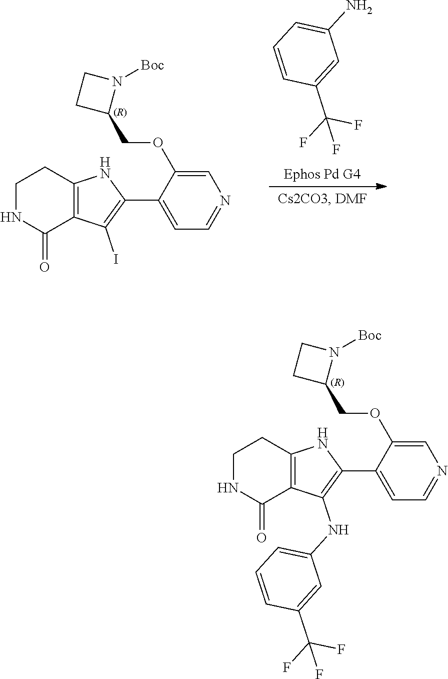

To a stirred mixture of tert-butyl (2R)-2-{[(4-{3-iodo-4-oxo-1H,5H,6H,7H-pyrrolo[3,2-c]pyridin-2-yl}pyridin-3-yl)oxy]methyl}azetidine-1-carboxylate (200 mg, 0.38 mmol, 1 equiv) and 3-(trifluoromethyl)aniline (122 mg, 0.76 mmol, 2 equiv) in DMF (2 mL) were added Ephos Pd G4 (35 mg, 0.038 mmol, 0.1 equiv) and Cs₂CO₃ (248 mg, 0.76 mmol, 2 equiv) in portions at room temperature under argon atmosphere. The resulting mixture was stirred for 1 h at 50° C. under argon atmosphere. Desired product could be detected by LCMS. The resulting mixture was concentrated under reduced pressure. The residue was purified by silica gel column chromatography, eluted with CH₂Cl₂/MeOH (20:1) to afford tert-butyl (2R)-2-({[4-(4-oxo-3-{[3-(trifluoromethyl)phenyl]amino}-1H,5H,6H,7H-pyrrolo[3,2-c]pyridin-2-yl)pyridin-3-yl]oxy}methyl)azetidine-1-carboxylate (267 mg, 125.55%) as a yellow solid.

LC-MS: [M+H]− found 558.00.

### 91.2. Synthesis of 2-{3-[(2R)-azetidin-2-ylmethoxy]pyridin-4-yl}-3-{[3-(trifluoromethyl)phenyl]amino}-1H,5H,6H,7H-pyrrolo[3,2-c]pyridin-4-one

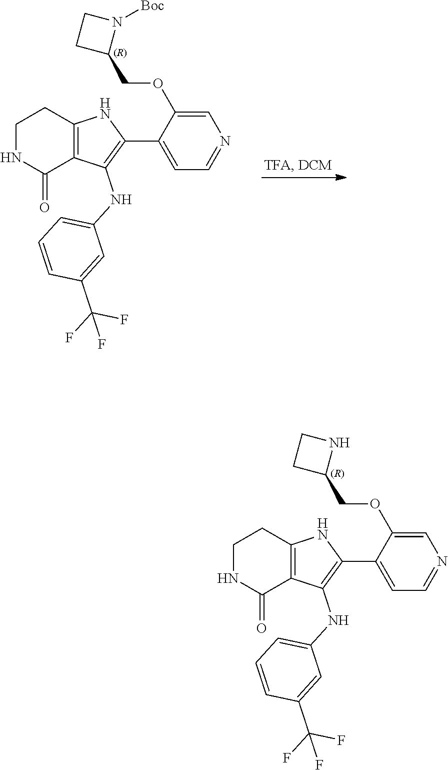

A mixture of tert-butyl (2R)-2-({[4-(4-oxo-3-{[3-(trifluoromethyl)phenyl]amino}-1H,5H,6H,7H-pyrrolo[3,2-c]pyridin-2-yl)pyridin-3-yl]oxy}methyl)azetidine-1-carboxylate (90 mg, 0.16 mmol, 1 equiv) in TFA (3 mL) and DCM (3 mL) was stirred for 1 h at room temperature under air atmosphere. Desired product could be detected by LCMS. The resulting mixture was concentrated under vacuum. This resulted in 2-{3-[(2R)-azetidin-2-ylmethoxy]pyridin-4-yl}-3-{[3-(trifluoromethyl)phenyl]amino}-1H,5H,6H,7H-pyrrolo[3,2-c]pyridin-4-one (70 mg, 94.80%) as a yellow oil.

LC-MS: [M+H]⁺ found 458.00.

### 91.3. Synthesis of 2-(3-{[(2R)-1-(but-2-ynoyl)azetidin-2-yl]methoxy}pyridin-4-yl)-3-{[3-(trifluoromethyl)phenyl]amino}-1H,5H,6H,7H-pyrrolo[3,2-c]pyridin-4-one

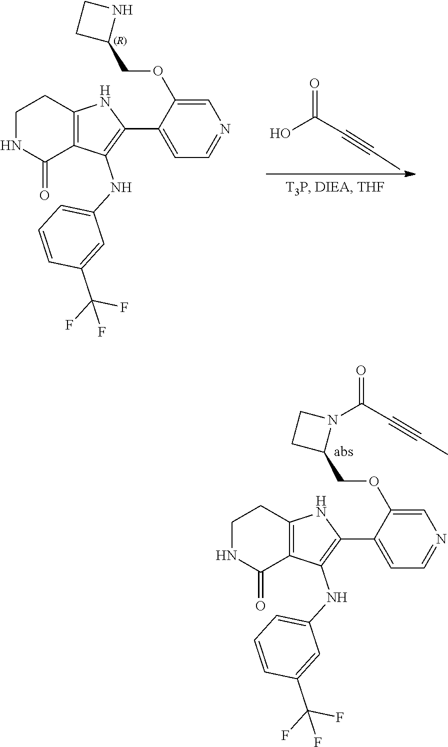

To a stirred mixture of 2-{3-[(2R)-azetidin-2-ylmethoxy]pyridin-4-yl}-3-{[3-(trifluoromethyl)phenyl]amino}-1H,5H,6H,7H-pyrrolo[3,2-c]pyridin-4-one (70 mg, 0.15 mmol, 1 equiv) and 2-butynoic acid (19 mg, 0.22 mmol, 1.5 equiv) in THF (3 mL) were added DIEA (197 mg, 1.53 mmol, 10 equiv) and T₃P (146 mg, 0.45 mmol, 3 equiv) in portions at 0° C. under air atmosphere. The resulting mixture was stirred for 1 h at room temperature under airatmosphere. Desired product could be detected by LCMS. The aqueous layer was extracted with CH₂Cl₂ (2×20 mL). The resulting mixture was concentrated under reduced pressure. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 24% B to 49% B in 9 min, 49% B; Wave Length: 254/220 nm; RT1(min): 8.85; Number Of Runs: 0) to afford 2-(3-{[(2R)-1-(but-2-ynoyl)azetidin-2-yl]methoxy}pyridin-4-yl)-3-{[3-(trifluoromethyl)phenyl]amino}-1H,5H,6H,7H-pyrrolo[3,2-c]pyridin-4-one (28.3 mg, 35.19%) as a light yellow solid.

LC-MS: [M+H]+ found 524.00.

¹H NMR (300 MHz, DMSO-d6) δ 11.48 (s, 1H), 8.37 (s, 1H), 8.00 (d, J=5.0 Hz, 1H), 7.83 (s, 1H), 7.41 (d, J=5.1 Hz, 1H), 7.27-7.04 (m, 2H), 6.86-6.74 (m, 3H), 4.82-4.71 (m, 1H), 4.51-4.29 (m, 2H), 4.16-4.02 (m, 2H), 3.41-3.34 (m, 2H), 2.96-2.80 (m, 2H), 2.15-20.2 (m, 4H).

### Example 92. 3-[(3-chloro-2-methoxyphenyl)amino]-2-(3-{[(2R)-1-[(2E)-4-(3-methoxyazetidin-1-yl)but-2-enoyl]azetidin-2-yl]methoxy}pyridin-4-yl)-1H,5H,6H,7H-pyrrolo[3,2-c]pyridin-4-one (compound 225)

### 92.1. Synthesis of (2E)-4-bromobut-2-enoyl chloride

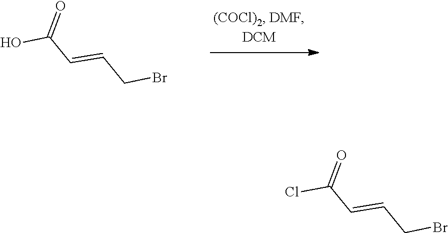 [SMILES: O=C(O)/C=C/CBr ; O=C(Cl)/C=C/CBr]

To a stirred mixture of 4-bromo-trans-crotonic acid (200 mg, 1.21 mmol, 1.00 equiv) and DMF 1 drop in DCM (5 mL) was added SOCl₂ (288 mg, 2.42 mmol, 2.00 equiv) dropwise at 0° C. The resulting mixture was stirred for 2h at 25° C. TLC (PE:EA=2:1) showed a new spot was detected. The resulting mixture was concentrated under reduced pressure to give the (2E)-4-bromobut-2-enoyl chloride (180 mg, 80.95%) as brown oil.

### 92.2. Synthesis of (R,E)-2-(3-((1-(4-bromobut-2-enoyl)azetidin-2-yl)methoxy)pyridin-4-yl)-3-((3-chloro-2-methoxyphenyl)amino)-1,5,6,7-tetrahydro-4H-pyrrolo[3,2-c]pyridin-4-one

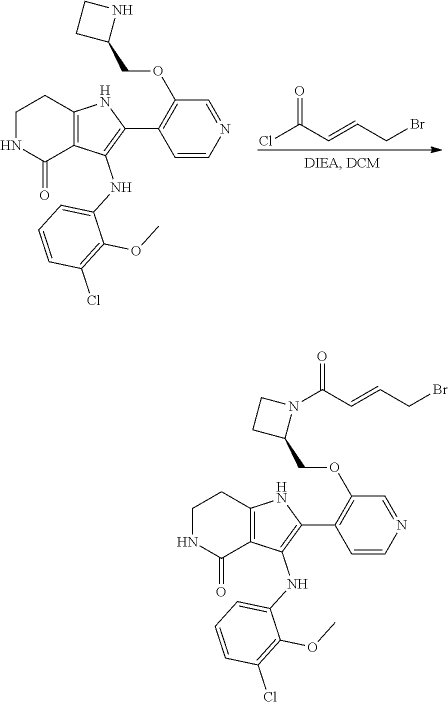 [SMILES: COc1c(Cl)cccc1Nc1c(-c2ccncc2OC[C@H]2CCN2)[nH]c2c1C(=O)NCC2 ; O=C(Cl)/C=C/CBr ; COc1c(Cl)cccc1Nc1c(-c2ccncc2OC[C@H]2CCN2C(=O)/C=C/CBr)[nH]c2c1C(=O)NCC2]

To a stirred mixture of 2-{3-[(2R)-azetidin-2-ylmethoxy]pyridin-4-yl}-3-[(3-chloro-2-methoxyphenyl)amino]-1H,5H,6H,7H-pyrrolo[3,2-c]pyridin-4-one (100 mg, 0.22 mmol, 1.00 equiv) and DIEA (85 mg, 0.66 mmol, 3.00 equiv) in DCM (2 mL) was added (2E)-4-bromobut-2-enoyl chloride (48 mg, 0.26 mmol, 1.20 equiv) dropwise at 0° C. The resulting mixture was stirred for 2h at 25° C. The resulting mixture was used in the next step directly without further purification.

LC-MS: (M+H)⁺ found: 600.

### 92.3. Synthesis of 3-[(3-chloro-2-methoxyphenyl)amino]-2-(3-{[(2R)-1-[(2E)-4-(3-methoxyazetidin-1-yl)but-2-enoyl]azetidin-2-yl]methoxy}pyridin-4-yl)-1H,5H,6H,7H-pyrrolo[3,2-c]pyridin-4-one

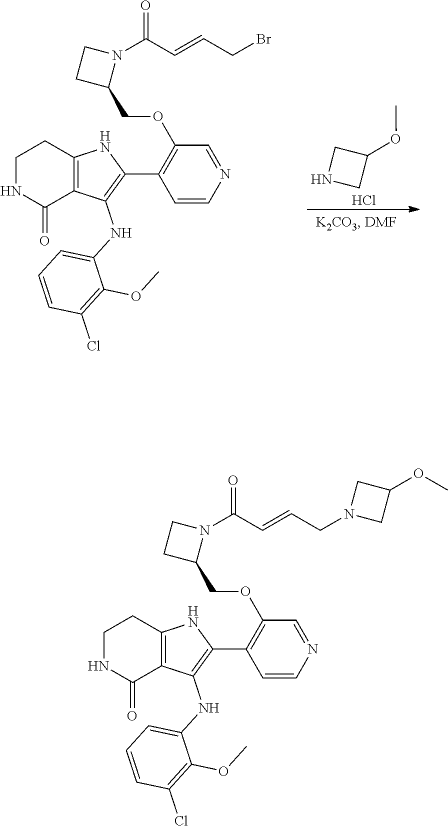

To a solution of 3-methoxyazetidine hydrochloride (32 mg, 0.26 mmol, 1.20 equiv) in DMF (0.5 mL) was added K₂CO₃ (90 mg, 0.65 mmol, 3.00 equiv) and the mixture was stirred for 30 min. Then the mixture was added to the solution of 2-(3-{[(2R)-1-[(2E)-4-bromobut-2-enoyl] azetidin-2-yl]methoxy}pyridin-4-yl)-3-[(3-chloro-2-methoxyphenyl)amino]-1H,5H,6H,7H-pyrrolo[3,2-c]pyridin-4-one (130 mg, 0.22 mmol, 1.00 equiv) in 2 mL DCM and stirred for 24h at room temperature. Desired product could be detected by LCMS. The reaction mixture was quenched by water and extracted with DCM (3*10 mL). The resulting mixture was concentrated under reduced pressure. The crude product was purified by Prep-HPLC with the following conditions (Column: XSelect CSH Fluoro Phenyl, 30*150 mm, 5 μm; Mobile Phase A: Water (10 mmol/L NH₄HCO₃), Mobile Phase B: ACN; Flow rate: 60 mL/min; Gradient: 39% B to 55% B in 8 min, 55% B; Wave Length: 254/220 nm; RT1(min): 8; Number Of Runs: 0) to afford 3-[(3-chloro-2-methoxyphenyl)amino]-2-(3-{[(2R)-1-[(2E)-4-(3-methoxyazetidin-1-yl)but-2-enoyl]azetidin-2-yl]methoxy}pyridin-4-yl)-1H,5H,6H,7H-pyrrolo[3,2-c]pyridin-4-one (28.3 mg, 21.55%) as a yellow solid.

LC-MS: (M+H)⁺ found: 607.15.

¹H NMR (300 MHz, Chloroform-d) δ 11.77 (s, 1H), 8.22 (s, 1H), 7.99 (d, 1H), 7.53 (s, 1H), 7.45 (d, 1H), 6.90-6.76 (m, 1H), 6.74-6.66 (m, 1H), 6.60 (t, 1H), 6.28-6.17 (m, 1H), 6.15-6.04 (m, 1H), 5.17 (t, 1H), 5.07-4.93 (m, 1H), 4.50 (t, 1H), 4.35-4.20 (m, 3H), 4.16-4.02 (m, 4H), 3.79-3.52 (m, 4H), 3.27 (s, 5H), 3.19-2.98 (m, 4H), 2.67-2.55 (m, 1H), 2.13 (m, 1H).

### Example 93. 3-[(3-chloro-2-methoxyphenyl)amino]-2-(3-{[(2R)-1-[(2E)-4-(pyrrolidin-1-yl)but-2-enoyl]azetidin-2-yl]methoxy}pyridin-4-yl)-1H,5H,6H,7H-pyrrolo[3,2-c]pyridin-4-one (compound 224)

### 93.1. Synthesis of tert-butyl (2R)-2-{[(4-cyanopyridin-3-yl)oxy]methyl}azetidine-1-carboxylate

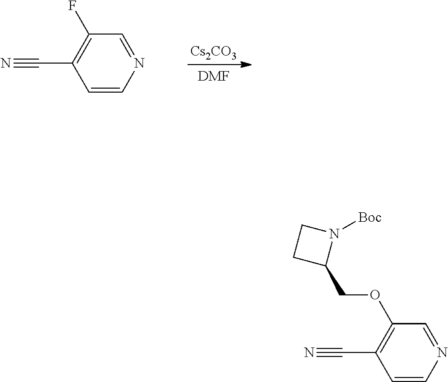

To a stirred solution of tert-butyl (2R)-2-(hydroxymethyl)azetidine-1-carboxylate (6.50 g, 34.71 mmol, 1.00 equiv) and 3-fluoropyridine-4-carbonitrile (4.24 g, 34.71 mmol, 1.00 equiv) in DMF (150 mL) was added Cs₂CO₃ (33.93 g, 104.14 mmol, 3.00 equiv) in portions at room temperature under argon atmosphere. The resulting mixture was stirred for 4h at 80 degrees C. under argon atmosphere. The mixture was allowed to cool down to room temperature. The resulting mixture was extracted with EtOAc (3×100 mL). The combined organic layers were washed with water (3×60 mL), dried over anhydrous Na₂SO₄. After filtration, the filtrate was concentrated under reduced pressure. The residue was purified by silica gel column chromatography, eluted with PE/EA (1:1) to afford tert-butyl (2R)-2-{[(4-cyanopyridin-3-yl)oxy]methyl}azetidine-1-carboxylate (10.00 g, 99.56%) as a yellow solid.

LC-MS: (M+H)⁺ found: 290.2

### 93.2. Synthesis of tert-butyl (2R)-2-([[4-(aminomethyl)pyridin-3-yl]oxy]methyl)azetidine-1-carboxylate

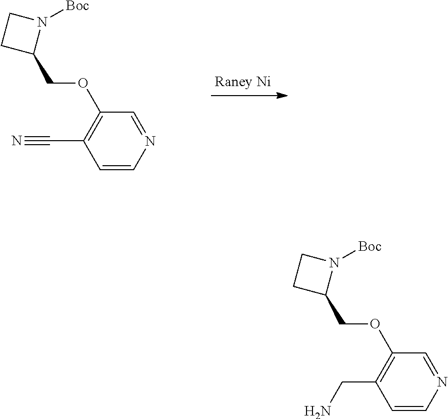 [SMILES: CC(C)(C)OC(=O)N1CC[C@@H]1COc1cnccc1C#N ; CC(C)(C)OC(=O)N1CC[C@@H]1COc1cnccc1CN]

To a stirred solution of tert-butyl (2R)-2-[[(4-cyanopyridin-3-yl)oxy]methyl]azetidine-1-carboxylate (10.50 g, 36.29 mmol, 1.00 equiv) in MeOH (100 mL) was added Ammonia (7.0 M Solution in MeOH, 100 mL, 700.00 mmol) and Raney Ni (4.66 g, 44 w/w %) at room temperature under hydrogen atmosphere. The resulting mixture was stirred for overnight at room temperature under hydrogen atmosphere. The resulting mixture was filtered, the filter cake was washed with MeOH, the filtrate was concentrated under reduced pressure. The residue was purified by silica gel column chromatography, eluted with CH₂Cl₂/MeOH (10:1) to afford tert-butyl (2R)-2-([[4-(aminomethyl)pyridin-3-yl]oxy]methyl)azetidine-1-carboxylate (10.00 g, 93.93%) as a yellow solid.

LC-MS: (M+H)⁺ found 294.2.

### 93.3. Synthesis of tert-butyl 4-{[(3-{[(2R)-1-(tert-butoxycarbonyl)azetidin-2-yl]methoxy}pyridin-4-yl)methyl]amino}-3-[(3-chloro-2-methoxyphenyl)carbamothioyl]-2-oxo-5,6-dihydropyridine-1-carboxylate

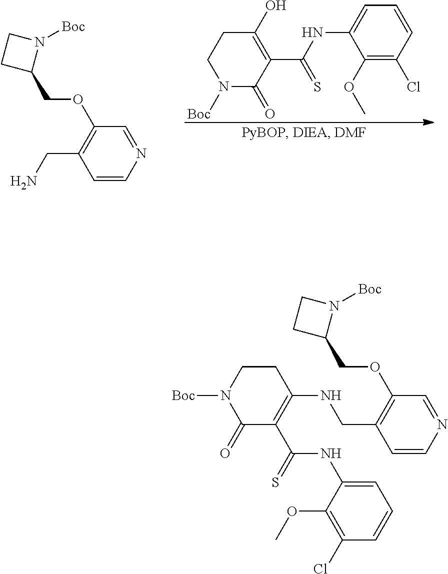 [SMILES: CC(C)(C)OC(=O)N1CC[C@@H]1COc1cnccc1CN ; COc1c(Cl)cccc1NC(=S)C1=C(O)CCN(C(=O)OC(C)(C)C)C1=O ; COc1c(Cl)cccc1NC(=S)C1=C(NCc2ccncc2OC[C@H]2CCN2C(=O)OC(C)(C)C)CCN(C(=O)OC(C)(C)C)C1=O]

To a stirred solution of tert-butyl (2R)-2-({[4-(aminomethyl)pyridin-3-yl]oxy}methyl)azetidine-1-carboxylate (10.00 g, 34.09 mmol, 1.00 equiv) and tert-butyl 3-[(3-chloro-2-methoxyphenyl)carbamothioyl]-4-hydroxy-2-oxo-5,6-dihydropyridine-1-carboxylate (14.07 g, 34.09 mmol, 1.00 equiv) and DIEA (13.22 g, 102.26 mmol, 3.00 equiv) in DMF (150 mL) were added PyBOP (26.61 g, 51.13 mmol, 1.50 equiv) in portions. The resulting mixture was stirred for overnight at room temperature under argon atmosphere. The resulting mixture was diluted with water (100 mL). The resulting mixture was extracted with EtOAc (3×100 mL). The combined organic layers were washed with water (3×100 mL), dried over anhydrous Na₂SO₄. After filtration, the filtrate was concentrated under reduced pressure. The residue was purified by silica gel column chromatography, eluted with PE/EA (1:2) to afford tert-butyl 4-{[(3-{[(2R)-1-(tert-butoxycarbonyl)azetidin-2-yl]methoxy}pyridin-4-yl)methyl]amino}-3-[(3-chloro-2-methoxyphenyl)carbamothioyl]-2-oxo-5,6-dihydropyridine-1-carboxylate (17.00 g, 72.46%) as a yellow solid.

LC-MS: (M+H)⁺ found 688.1.

### 93.4. Synthesis of tert-butyl (2R)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]azetidine-1-carboxylate

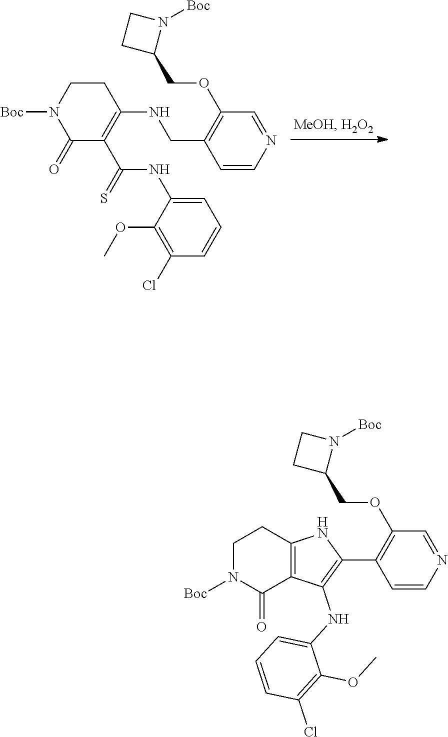 [SMILES: COc1c(Cl)cccc1NC(=S)C1=C(NCc2ccncc2OC[C@H]2CCN2C(=O)OC(C)(C)C)CCN(C(=O)OC(C)(C)C)C1=O ; COc1c(Cl)cccc1Nc1c(-c2ccncc2OC[C@H]2CCN2C(=O)OC(C)(C)C)[nH]c2c1C(=O)N(C(=O)OC(C)(C)C)CC2]

A solution of tert-butyl 4-{[(3-{[(2R)-1-(tert-butoxycarbonyl)azetidin-2-yl]methoxy}pyridin-4-yl)methyl]amino}-3-[(3-chloro-2-methoxyphenyl)carbamothioyl]-2-oxo-5,6-dihydropyridine-1-carboxylate (8.00 g, 10.461 mmol, 1.00 equiv, 90%) and H₂O₂ (30 w/w %, 1.54 g, 13.60 mmol, 1.30 equiv) in MeOH (100 mL) was stirred for 1 h at 80° C. under N₂ atmosphere. Desired product could be detected by LCMS. The mixture was allowed to cool down to room temperature. The resulting mixture was diluted with water (100 mL). The resulting mixture was extracted with CH₂Cl₂ (3×100 mL). The combined organic layers were washed with water (3×50 mL), dried over anhydrous Na₂SO₄. After filtration, the filtrate was concentrated under reduced pressure. The crude product was used in the next step directly without further purification. This resulted in tert-butyl (2R)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]azetidine-1-carboxylate (6.3 g, 58.00%) as a yellow solid.

### 93.5. Synthesis of 2-{3-[(2R)-azetidin-2-ylmethoxy]pyridin-4-yl}-3-[(3-chloro-2-methoxyphenyl)amino]-1H,5H,6H,7H-pyrrolo[3,2-c]pyridin-4-one

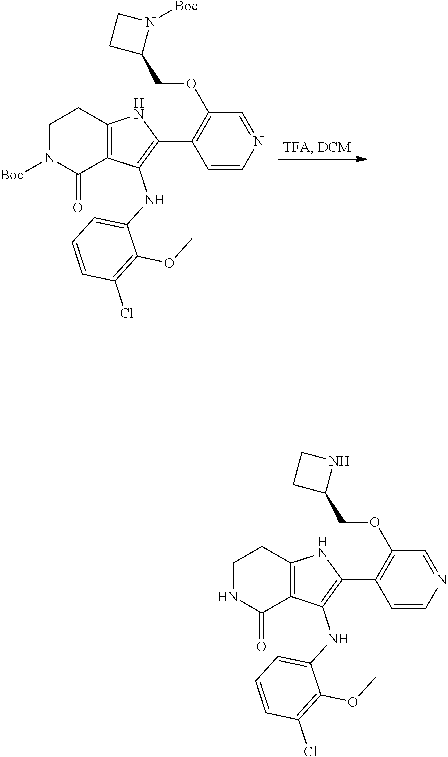 [SMILES: COc1c(Cl)cccc1Nc1c(-c2ccncc2OC[C@H]2CCN2C(=O)OC(C)(C)C)[nH]c2c1C(=O)N(C(=O)OC(C)(C)C)CC2 ; COc1c(Cl)cccc1Nc1c(-c2ccncc2OC[C@H]2CCN2)[nH]c2c1C(=O)NCC2]

To a stirred solution of tert-butyl (2R)-2-[({4-[5-(tert-butoxycarbonyl)-3-[(3-chloro-2-methoxyphenyl)amino]-4-oxo-1H,6H,7H-pyrrolo[3,2-c]pyridin-2-yl]pyridin-3-yl}oxy)methyl]azetidine-1-carboxylate (6.30 g, 9.63 mmol, 1.00 equiv) in CH₂Cl₂ (60 mL) were added TFA (12 mL) in portions at 0° C. under argon atmosphere. The resulting mixture was stirred for 6h at room temperature under argon atmosphere. Desired product could be detected by LCMS. The mixture was basified to pH 8 with saturated NaHCO₃(aq.). The resulting mixture was extracted with CH₂Cl₂ (3×100 mL). The combined organic layers were washed with brine (3×30 mL), dried over anhydrous Na₂SO₄. After filtration, the filtrate was concentrated under reduced pressure. The residue was purified by silica gel column chromatography, eluted with CH₂Cl₂/MeOH (10:1) to afford 2-{3-[(2R)-azetidin-2-ylmethoxy]pyridin-4-yl}-3-[(3-chloro-2-methoxyphenyl)amino]-1H,5H,6H,7H-pyrrolo[3,2-c]pyridin-4-one (2.8 g, 64.05%) as a yellow solid.

LC-MS: (M+H)⁺ found: 454.05.

### 93.6. Synthesis of (R,E)-2-(3-((1-(4-bromobut-2-enoyl)azetidin-2-yl)methoxy)pyridin-4-yl)-3-((3-chloro-2-methoxyphenyl)amino)-1,5,6,7-tetrahydro-4H-pyrrolo[3,2-c]pyridin-4-one

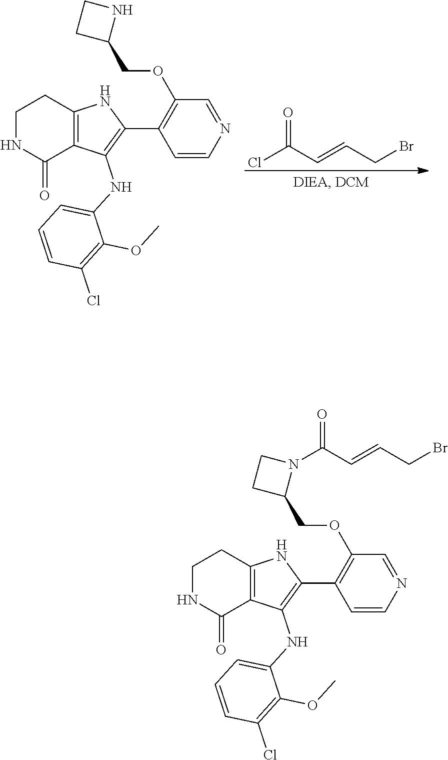 [SMILES: COc1c(Cl)cccc1Nc1c(-c2ccncc2OC[C@H]2CCN2)[nH]c2c1C(=O)NCC2 ; O=C(Cl)/C=C/CBr ; COc1c(Cl)cccc1Nc1c(-c2ccncc2OC[C@H]2CCN2C(=O)/C=C/CBr)[nH]c2c1C(=O)NCC2]

To a stirred solution of 2-{3-[(2R)-azetidin-2-ylmethoxy]pyridin-4-yl}-3-[(3-chloro-2-methoxyphenyl)amino]-1H,5H,6H,7H-pyrrolo[3,2-c]pyridin-4-one (60 mg, 0.13 mmol, 1.00 equiv) and DIEA (51 mg, 0.39 mmol, 3 equiv) in DCM (3 mL) were added (2E)-4-bromobut-2-enoyl chloride (29 mg, 0.15 mmol, 1.2 equiv) at 0° C. under nitrogen atmosphere. The resulting mixture was stirred for 0.5h at 0° C. under nitrogen atmosphere. The resulting mixture was used in the next step directly without further purification.

### 93.7. Synthesis of 3-[(3-chloro-2-methoxyphenyl)amino]-2-(3-{[(2R)-1-[(2E)-4-(pyrrolidin-1-yl)but-2-enoyl]azetidin-2-yl]methoxy}pyridin-4-yl)-1H,5H,6H,7H-pyrrolo[3,2-c]pyridin-4-one

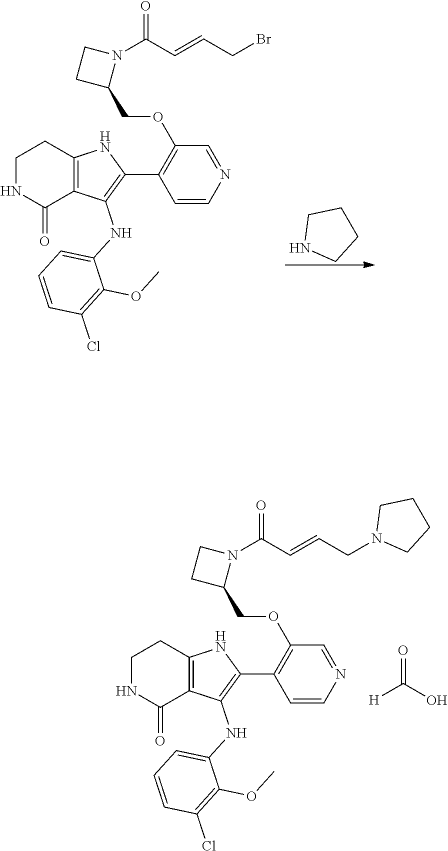

To the last step resulting mixture was added pyrrolidine (24 mg, 0.33 mmol, 3.00 equiv) dropwise at 0° C. The resulting mixture was stirred for additional overnight at room temperature. Desired product could be detected by LCMS. The resulting mixture was extracted with CH₂Cl₂:MeOH (3×50 mL). The combined organic layers were dried over anhydrous Na₂SO₄. After filtration, the filtrate was concentrated under reduced pressure and dissolved in DMSO. The crude product (100 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 11% B to 22% B in 7 min, 22% B; Wave Length: 254/220 nm; RT1(min): 6.5; Number Of Runs: 0) to afford 3-[(3-chloro-2-methoxyphenyl)amino]-2-(3-{[(2R)-1-[(2E)-4-(pyrrolidin-1-yl)but-2-enoyl]azetidin-2-yl]methoxy}pyridin-4-yl)-1H,5H,6H,7H-pyrrolo[3,2-c]pyridin-4-one (22.6 mg, 34.71%) as a yellow solid.

LC-MS: (M+H)⁺ found 591.00.

¹H NMR (400 MHz, Chloroform-d) δ 11.77 (s, 1H), 8.36 (s, 1H), 8.24 (s, 1H), 7.97 (d, 1H), 7.55 (s, 1H), 7.46 (d, 1H), 6.96-6.91 (m, 1H), 6.71-6.93 (m, 1H), 6.61 (t, 1H), 6.32-6.12 (m, 2H), 5.51 (t, 1H), 5.01 (q, 1H), 4.50 (t, 1H), 4.38-4.17 (m, 3H), 4.08 (s, 3H), 3.68-3.47 (m, 4H), 3.21-3.01 (m, 2H), 2.89 (q, 4H), 2.63-2.60 (m, 1H), 2.15-2.13 (m, 1H), 1.96-1.95 (m, 4H).

### Example 94. 3-[(3-chloro-2-methoxyphenyl)amino]-2-(3-{[(2R)-1-[(2E)-4-[(3R)-3-methoxypyrrolidin-1-yl]but-2-enoyl]azetidin-2-yl]methoxy}pyridin-4-yl)-1H,5H,6H,7H-pyrrolo[3,2-c]pyridin-4-one (compound 223)

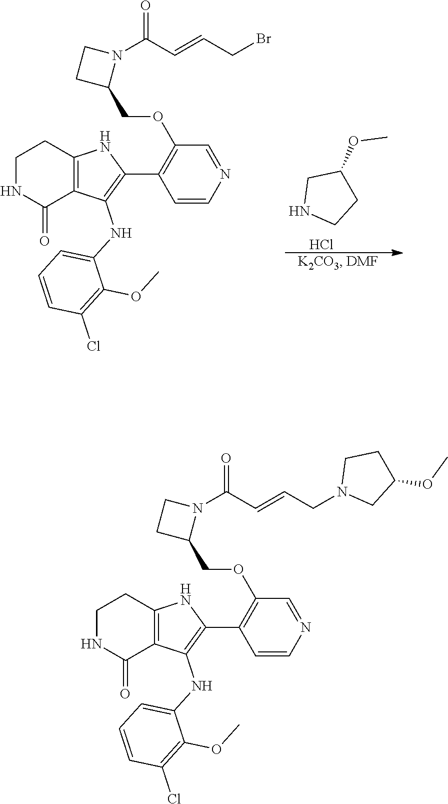

To a solution of (3R)-3-methoxypyrrolidine hydrochloride (36 mg, 0.26 mmol, 1.20 equiv) in DMF (1 mL) was added K₂CO₃ (90 mg, 0.65 mmol, 3.00 equiv) and the mixture was stirred for 30 min. Then the mixture was added to the solution of 2-(3-{[(2R)-1-[(2E)-4-bromobut-2-enoyl]azetidin-2-yl]methoxy}pyridin-4-yl)-3-[(3-chloro-2-methoxyphenyl)amino]-1H,5H,6H,7H-pyrrolo[3,2-c]pyridin-4-one (130 mg, 0.22 mmol, 1.00 equiv) in 2 mL DCM and stirred for 4 hrs at room temperature. Desired product could be detected by LCMS. The reaction mixture was quenched by water and extracted with DCM (3*10 mL). The resulting mixture was concentrated under reduced pressure. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 18% B to 40% B in 9 min, 40% B; Wave Length: 254/220 nm; RT1(min): 9.67; Number Of Runs: 0) to afford 3-[(3-chloro-2-methoxyphenyl)amino]-2-(3-{[(2R)-1-[(2E)-4-[(3R)-3-methoxypyrrolidin-1-yl]but-2-enoyl]azetidin-2-yl]methoxy}pyridin-4-yl)-1H,5H,6H,7H-pyrrolo[3,2-c]pyridin-4-one (19.7 mg, 14.60%) as a yellow solid.

LC-MS: (M+H)⁺ found: 621.20.

¹H NMR (400 MHz, Chloroform-d) δ 11.69 (s, 1H), 8.16 (s, 1H), 7.92 (d, 1H), 7.52-7.33 (m, 2H), 6.97-6.83 (m, 1H), 6.67-6.48 (m, 2H), 6.22-5.98 (m, 2H), 5.11 (s, 1H), 5.00-4.88 (m, 1H), 4.43 (t, 1H), 4.29-4.10 (m, 3H), 4.02 (s, 3H), 3.87 (d, 1H), 3.52 (m, 2H), 3.27 (s, 2H), 3.22 (s, 3H), 3.15-2.99 (m, 2H), 2.83-2.43 (m, 5H), 2.12-1.97 (m, 2H), 1.88-1.78

### Example 95. 3-[(3-chloro-2-methoxyphenyl)amino]-2-(3-{[(2R)-1-[(2E)-4-(3,3-difluoropyrrolidin-1-yl)but-2-enoyl]azetidin-2-yl]methoxy}pyridin-4-yl)-1H,5H,6H,7H-pyrrolo[3,2-c]pyridin-4-one (compound 221)

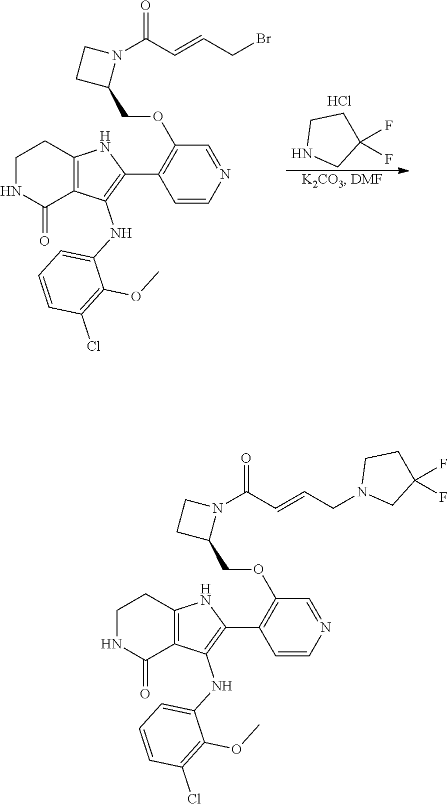

To a solution of 3,3-difluoropyrrolidine hydrochloride (28 mg, 0.20 mmol, 1.20 equiv) in DMF (0.5 mL) was added K₂CO₃ (69 mg, 0.49 mmol, 3.00 equiv) at 0° C. and the mixture was stirred for 30 min. Then added to the last step resulting mixture dropwise at 0° C. The resulting mixture was stirred for additional 48 h at room temperature. The resulting mixture was extracted with CH₂Cl₂:MeOH (3×50 mL). The combined organic layers were dried over anhydrous Na₂SO₄. After filtration, the filtrate was concentrated under reduced pressure and dissolved in DMSO. The crude product (100 mg) was purified by Prep-HPLC with the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 22% B to 52% B in 10 min, 52% B; Wave Length: 220/254 nm; RT1(min): 10.55; Number Of Runs: 0) to afford 3-[(3-chloro-2-methoxyphenyl)amino]-2-(3-{[(2R)-1-[(2E)-4-(3,3-difluoropyrrolidin-1-yl)but-2-enoyl]azetidin-2-yl]methoxy}pyridin-4-yl)-1H,5H,6H,7H-pyrrolo[3,2-c]pyridin-4-one (36.8 mg, 26.64%) as a light yellow solid.

LC-MS: (M+H)⁺ found 627.15.

¹H NMR (400 MHz, Chloroform-d) δ 11.78 (s, 1H), 8.25 (s, 1H), 8.01 (d, 1H), 7.57 (s, 1H), 7.47 (d, 1H), 6.92-6.86 (m, 1H), 6.72-6.69 (m, 1H), 6.63 (t, 1H), 6.24-6.21 (m, 1H), 6.15-6.11 (m, 1H), 5.21 (d, 1H), 5.04 (q, 1H), 4.53 (t, 1H), 4.45-4.18 (m, 3H), 4.11 (s, 3H), 3.62-3.57 (m, 2H), 3.32-3.12 (m, 2H), 3.25-3.07 (m, 2H), 2.94-2.88 (m, 2H), 2.81 (t, 2H), 2.65-2.61 (m, 1H), 2.34-2.26 (m, 2H), 2.17-2.15 (m, 1H).

### Example 96. 3-[(3-chloro-2-methoxyphenyl)amino]-2-(3-{[(2R)-1-[(2E)-4-(3-fluoroazetidin-1-yl)but-2-enoyl]azetidin-2-yl]methoxy}pyridin-4-yl)-1H,5H,6H,7H-pyrrolo[3,2-c]pyridin-4-one (compound 219)

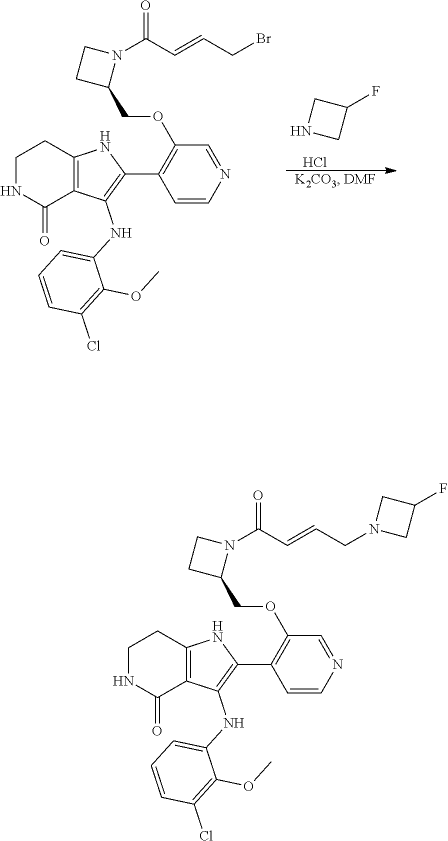

To a solution of 3-fluoroazetidine hydrochloride (28.96 mg, 0.259 mmol, 1.2 equiv) in DMF (1 mL) was added K₂CO₃ (90 mg, 0.65 mmol, 3.00 equiv) and the mixture was stirred for 30 min. Then the mixture was added to the solution of 2-(3-{[(2R)-1-[(2E)-4-bromobut-2-enoyl]azetidin-2-yl]methoxy}pyridin-4-yl)-3-[(3-chloro-2-methoxyphenyl)amino]-1H,5H,6H,7H-pyrrolo[3,2-c]pyridin-4-one (130 mg, 0.22 mmol, 1.00 equiv) in 2 mL DCM and stirred for 24h at room temperature. Desired product could be detected by LCMS. The reaction mixture was quenched by water and extracted with DCM (3*10 mL). The resulting mixture was concentrated under reduced pressure. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 22% B to 46% B in 9 min, 46% B; Wave Length: 254/220 nm; RT1(min): 8.85; Number Of Runs: 0) to afford 3-[(3-chloro-2-methoxyphenyl)amino]-2-(3-{[(2R)-1-[(2E)-4-(3-fluoroazetidin-1-yl)but-2-enoyl]azetidin-2-yl]methoxy}pyridin-4-yl)-1H,5H,6H,7H-pyrrolo[3,2-c]pyridin-4-one (13.6 mg, 10.50%) as a yellow solid.

LC-MS: (M+H)⁺ found: 595.15.

¹H NMR (300 MHz, Chloroform-d) δ 11.79 (s, 1H), 8.23 (s, 1H), 7.97 (d, 1H), 7.56 (s, 1H), 7.44 (d, 1H), 6.91-6.75 (m, 1H), 6.75-6.65 (m, 1H), 6.60 (t, 1H), 6.27-6.14 (m, 1H), 6.13-5.99 (m, 1H), 5.33-4.92 (m, 3H), 4.50 (t, 1H), 4.38-4.17 (m, 3H), 4.08 (s, 3H), 3.81-3.63 (m, 2H), 3.64-3.51 (m, 2H), 3.38-3.06 (m, 6H), 2.71-2.49 (m, 1H), 2.21-2.05 (m, 1H).

### Example 97. rel-3-[(3-fluoro-2-methoxyphenyl)amino]-2-(3-{2-[(2R)-2-methyl-1-(prop-2-enoyl)azetidin-2-yl]ethynyl}pyridin-4-yl)-1H,5H,6H,7H-pyrrolo[3,2-c]pyridin-4-one (compound 244)

### 97.1. Synthesis of tert-butyl 2-formyl-2-methylazetidine-1-carboxylate

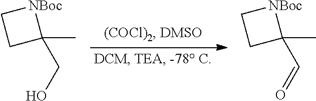

To a stirred solution of (COCl)₂ (757 mg, 5.96 mmol, 1.20 equiv) in DCM (50 mL) were added DMSO (0.9 mL, 12.42 mmol, 2.50 equiv) dropwise at −78° C. under nitrogen atmosphere. The resulting mixture was stirred for 30 min at −78° C. under nitrogen atmosphere. To the above mixture was added tert-butyl 2-(hydroxymethyl)-2-methylazetidine-1-carboxylate (1 g, 4.97 mmol, 1.00 equiv) dropwise at −78° C. The resulting mixture was stirred for additional 30 min at −78° C. Desired product could be detected by TLC. To the above mixture was added TEA (3.5 mL, 24.85 mmol, 5.00 equiv) at −78° C. The resulting mixture was stirred for additional 30 min at 0° C. The resulting mixture was extracted with CH₂Cl₂ (3×50 mL). The combined organic layers were washed with water (3×50 mL), dried over anhydrous Na₂SO₄. After filtration, the filtrate was concentrated under reduced pressure. The crude product tert-butyl 2-formyl-2-methylazetidine-1-carboxylate (1 g) was used in the next step directly without further purification.

### 97.2. Synthesis of tert-butyl 2-ethynyl-2-methylazetidine-1-carboxylate

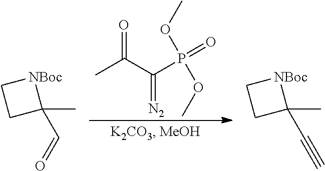

To a stirred solution of tert-butyl 2-formyl-2-methylazetidine-1-carboxylate (1 g, 5.02 mmol, 1.00 equiv) in MeOH (10 mL) were added K₂CO₃ (1.4 g, 10.04 mmol, 2.00 equiv) and seyferth-gilbert homologation (1.15 mg, 6.02 mmol, 1.2 equiv) at 0° C. under nitrogen atmosphere. The resulting mixture was stirred for 1 h at 0° C. under nitrogen atmosphere. Desired product could be detected by TLC. The reaction was quenched by the addition of Potassium sodium tartrate (aq.) (5 mL) at 0° C. The resulting mixture was extracted with EtOAc (3×30 mL). The combined organic layers were dried over anhydrous Na₂SO₄. After filtration, the filtrate was concentrated under reduced pressure. The residue was purified by silica gel column chromatography, eluted with PE/EA (10:1) to afford tert-butyl 2-ethynyl-2-methylazetidine-1-carboxylate (700 mg, 71.43%) as a colorless oil.

### 97.3. Synthesis of tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylazetidine-1-carboxylate

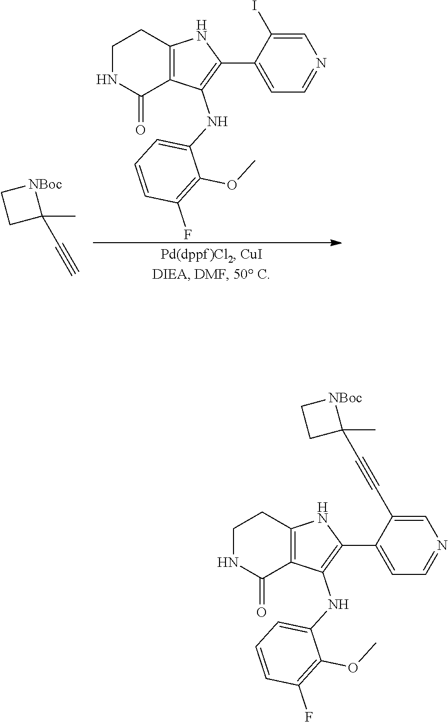 [SMILES: C#CC1(C)CCN1C(=O)OC(C)(C)C ; COc1c(F)cccc1Nc1c(-c2ccncc2I)[nH]c2c1C(=O)NCC2 ; COc1c(F)cccc1Nc1c(-c2ccncc2C#CC2(C)CCN2C(=O)OC(C)(C)C)[nH]c2c1C(=O)NCC2]

To a stirred solution of 3-[(3-fluoro-2-methoxyphenyl)amino]-2-(3-iodopyridin-4-yl)-1H,5H,6H,7H-pyrrolo[3,2-c]pyridin-4-one (500 mg, 1.05 mmol, 1.00 equiv) and tert-butyl 2-ethynyl-2-methylazetidine-1-carboxylate (408 mg, 2.09 mmol, 2.00 equiv) in DMF (5 mL) were added Pd(dppf)Cl₂CH₂Cl₂ (426 mg, 0.52 mmol, 0.50 equiv) and DIEA (676 mg, 5.23 mmol, 5.00 equiv) under nitrogen atmosphere. The resulting mixture was stirred for 4h at 50° C. under nitrogen atmosphere. The residue was purified by reverse flash chromatography with the following conditions: column, C18 spherical column; mobile phase, ACN in water, 0% to 100% gradient in 20 min; detector, UV 254 nm, to afford tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylazetidine-1-carboxylate (280 mg, 49.09%) as a yellow solid.

LC-MS: (M+H)⁺ found: 546.20

### 97.4. Synthesis of 2-{3-[2-(azetidin-2-yl)ethynyl]pyridin-4-yl}-3-[(3-fluoro-2-methoxyphenyl)amino]-1H,5H,6H,7H-pyrrolo[3,2-c]pyridin-4-one

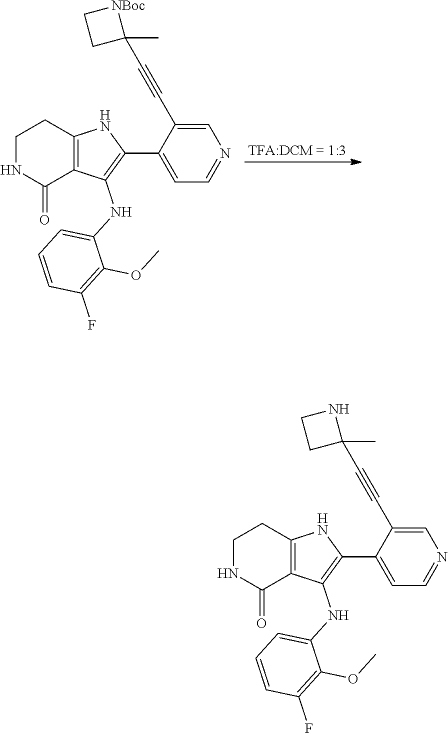 [SMILES: COc1c(F)cccc1Nc1c(-c2ccncc2C#CC2(C)CCN2C(=O)OC(C)(C)C)[nH]c2c1C(=O)NCC2 ; COc1c(F)cccc1Nc1c(-c2ccncc2C#CC2(C)CCN2)[nH]c2c1C(=O)NCC2]

To a stirred solution of tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylazetidine-1-carboxylate (350 mg, 0.64 mmol, 1.00 equiv) in DCM (3 mL) was added TFA (1 mL) dropwise at room temperature under nitrogen atmosphere. The resulting mixture was stirred for overnight at room temperature under nitrogen atmosphere. The resulting mixture was concentrated under vacuum to afford the crude product 2-{3-[2-(azetidin-2-yl)ethynyl]pyridin-4-yl}-3-[(3-fluoro-2-methoxyphenyl)amino]-1H,5H,6H,7H-pyrrolo[3,2-c]pyridin-4-one (500 mg) as a yellow oil.

LC-MS: M+H found: 446.10.

### 97.5. Synthesis of 3-[(3-fluoro-2-methoxyphenyl)amino]-2-(3-{2-[2-methyl-1-(prop-2-enoyl)azetidin-2-yl]ethynyl}pyridin-4-yl)-1H,5H,6H,7H-pyrrolo[3,2-c]pyridin-4-one

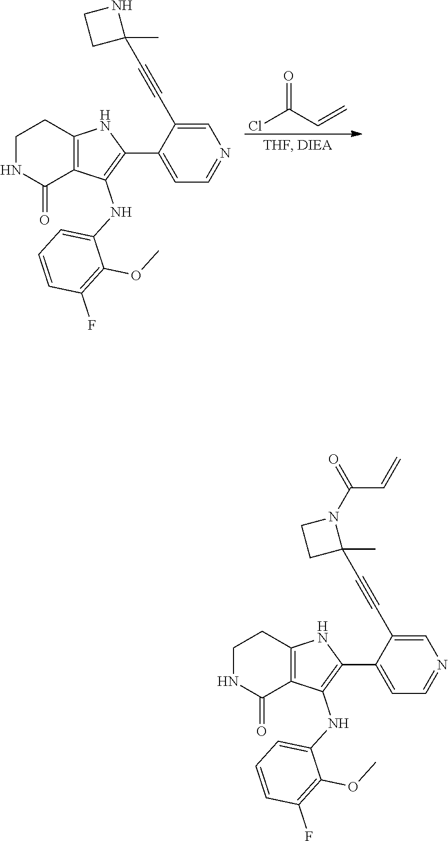

To a stirred solution of 3-[(3-fluoro-2-methoxyphenyl)amino]-2-{3-[2-(2-methylazetidin-2-yl)ethynyl]pyridin-4-yl}-1H,5H,6H,7H-pyrrolo[3,2-c]pyridin-4-one (250 mg, 0.56 mmol, 1.00 equiv) in THF (3 mL) were added NaHCO₃aq. (0.5 mL) and acryloyl chloride (46 mg, 0.51 mmol, 0.90 equiv) dropwise at 0° C. under nitrogen atmosphere. The resulting mixture was stirred for 1 h at room temperature under nitrogen atmosphere. The resulting mixture was extracted with CH₂Cl₂:MeOH (3×20 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford the crude product. The crude product was concentrated under vacuum and dissolved in DMSO. The crude product (100 mg) was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 18% B to 35% B in 11 min, 35% B; Wave Length: 254/220 nm; RT1(min): 10.38; Number Of Runs: 0) to afford 3-[(3-fluoro-2-methoxyphenyl)amino]-2-(3-{2-[2-methyl-1-(prop-2-enoyl)azetidin-2-yl]ethynyl}pyridin-4-yl)-1H,5H,6H,7H-pyrrolo[3,2-c]pyridin-4-one (43 mg, 15.34%) as yellow solid.

LC-MS: M+H found: 500.10

### 97.6. Synthesis of rel-3-[(3-fluoro-2-methoxyphenyl)amino]-2-(3-{2-[(2R)-2-methyl-1-(prop-2-enoyl)azetidin-2-yl]ethynyl}pyridin-4-yl)-1H,5H,6H,7H-pyrrolo[3,2-c]pyridin-4-one

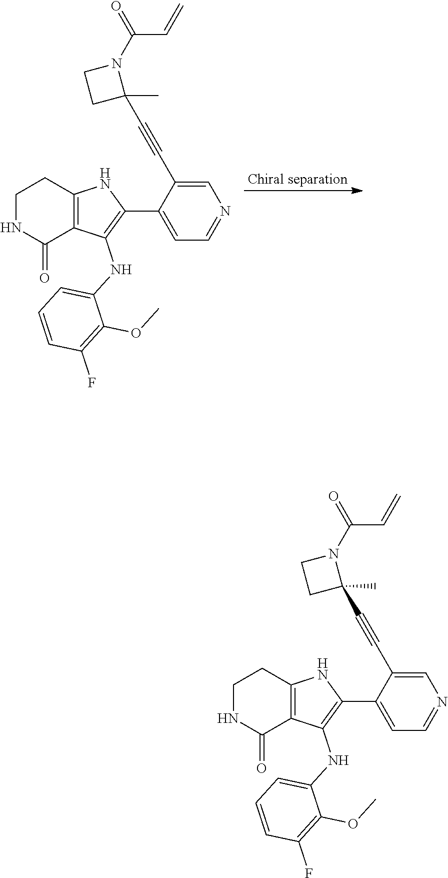 [SMILES: C=CC(=O)N1CCC1(C)C#Cc1cnccc1-c1[nH]c2c(c1Nc1cccc(F)c1OC)C(=O)NCC2 ; C=CC(=O)N1CC[C@]1(C)C#Cc1cnccc1-c1[nH]c2c(c1Nc1cccc(F)c1OC)C(=O)NCC2]

The 3-[(3-fluoro-2-methoxyphenyl)amino]-2-(3-{2-[2-methyl-1-(prop-2-enoyl)azetidin-2-yl]ethynyl}pyridin-4-yl)-1H,5H,6H,7H-pyrrolo[3,2-c]pyridin-4-one (43 mg, 0.086 mmol, 1.00 equiv) was purified by CHIRAL-IPLC with the following conditions (Column: CHIRALPAK ID-3, 4.6*50 mm, 3 m; Mobile Phase A: (Hex:DCM=3:1) (0.1% DEA):IPA=90:10; Flow rate: 1 mL/min; Gradient: 0% B to 0% B; Injection Volume: 5 ul mL) to afford rel-3-[(3-fluoro-2-methoxyphenyl)amino]-2-(3-{2-[(2R)-2-methyl-1-(prop-2-enoyl)azetidin-2-yl]ethynyl}pyridin-4-yl)-1H,5H,6H,7H-pyrrolo[3,2-c]pyridin-4-one (15.1 mg, 35.12%) as a yellow solid.

LC-MS: (M+H)⁺ found 500.10.

¹H NMR (400 MHz, Chloroform-d) δ 9.52 (brs, 1H), 8.80 (s, 1H), 8.27-8.21 (m, 2H), 7.21 (d, J=5.9 Hz, 1H), 6.77-6.73 (m, 1H), 6.58-6.55 (m, 1H), 6.27 (t, J=6.1 Hz, 1H), 6.15-6.11 (m, 1H), 5.94-5.88 (m, 1H), 5.57-5.55 (m, 1H), 5.42 (s, 1H), 3.99 (d, J=1.3 Hz, 3H), 3.63-3.40 (m, 3H), 3.15-2.82 (m, 3H), 2.25-2.17 (m, 1H), 2.01-1.95 (m, 1H), 1.82 (s, 3H).

### Example 98. rel-3-[(3-fluoro-2-methoxyphenyl)amino]-2-(3-{2-[(2R)-2-methyl-1-(prop-2-enoyl)azetidin-2-yl]ethynyl}pyridin-4-yl)-1H,5H,6H,7H-pyrrolo[3,2-c]pyridin-4-one (compound 243)

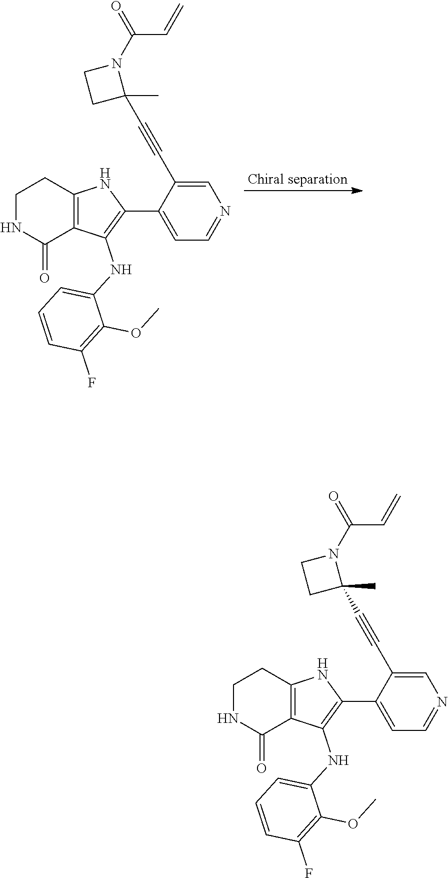 [SMILES: C=CC(=O)N1CCC1(C)C#Cc1cnccc1-c1[nH]c2c(c1Nc1cccc(F)c1OC)C(=O)NCC2 ; C=CC(=O)N1CC[C@@]1(C)C#Cc1cnccc1-c1[nH]c2c(c1Nc1cccc(F)c1OC)C(=O)NCC2]

The 3-[(3-fluoro-2-methoxyphenyl)amino]-2-(3-{2-[2-methyl-1-(prop-2-enoyl)azetidin-2-yl]ethynyl}pyridin-4-yl)-1H,5H,6H,7H-pyrrolo[3,2-c]pyridin-4-one (43 mg, 0.09 mmol, 1.00 equiv) was purified by CHIRAL-HPLC with the following conditions (Column: CHIRALPAK ID-3, 4.6*50 mm, 3 m; Mobile Phase A: (Hex:DCM=3:1)(0.1% DEA):IPA=90:10; Flow rate: 1 mL/min; Gradient: 0% B to 0% B; Injection Volume: 5 ul mL) to afford rel-3-[(3-fluoro-2-methoxyphenyl)amino]-2-(3-{2-[(2R)-2-methyl-1-(prop-2-enoyl)azetidin-2-yl]ethynyl}pyridin-4-yl)-1H,5H,6H,7H-pyrrolo[3,2-c]pyridin-4-one (14.3 mg, 33.26%) as a yellow solid.

LC-MS: (M+H)⁺ found 500.10.

¹H NMR (400 MHz, Chloroform-d) δ 9.42 (s, 1H), 8.73 (s, 1H), 8.40-8.19 (m, 2H), 7.15 (d, 1H), 6.78-6.74 (m, 1H), 6.57-6.52 (m, 1H), 6.28 (t, 1H), 6.15-6.10 (m, 1H), 5.95-5.88 (m, 1H), 5.56-5.52 (m, 1H), 5.43 (s, 1H), 3.98 (d, 3H), 3.61-3.38 (m, 3H), 3.12-2.79 (m, 3H), 2.23-2.16 (m, 1H), 2.04-1.95 (m, 1H), 1.81 (s, 3H).

### Example 99. 3-[(3-fluoro-2-methoxyphenyl)amino]-2-(3-{[(2S)-1-(2-fluoroprop-2-enoyl)pyrrolidin-2-yl]methoxy}pyridin-4-yl)-1H,5H,6H,7H-pyrrolo[3,2-c]pyridin-4-one (compound 287)

### 99.1. Synthesis of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate

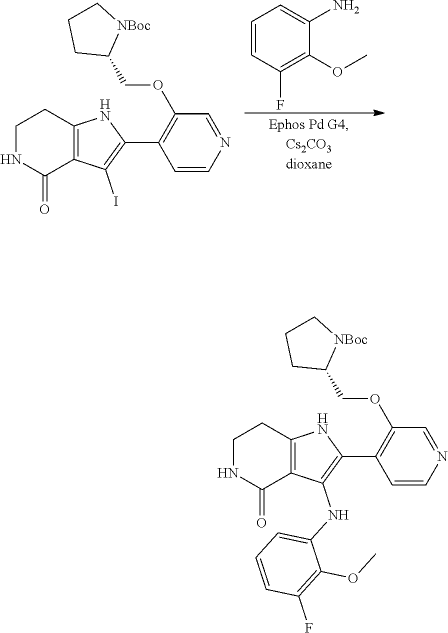

To a stirred mixture of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (400 mg, 0.74 mmol, 1.00 equiv) and 3-fluoro-2-methoxyaniline (210 mg, 1.49 mmol, 2.00 equiv) in DMF (5 mL) were added EPhos Pd G4 (68 mg, 0.07 mmol, 0.10 equiv) and Cs₂CO₃ (484 mg, 1.49 mmol, 2.00 equiv) under argon atmosphere. The resulting mixture was stirred for 2h at 50° C. under argon atmosphere. The resulting mixture was filtered, the filter cake was washed with DCM (2×10 mL). The filtrate was concentrated under reduced pressure. The residue was purified by silica gel column chromatography, eluted with CH₂C2/MeOH (20:1) to afford tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (310 mg, 75.64%) as a brown solid.

LC-MS: (M+H)⁺ found: 552.20

### 99.2. Synthesis of 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one

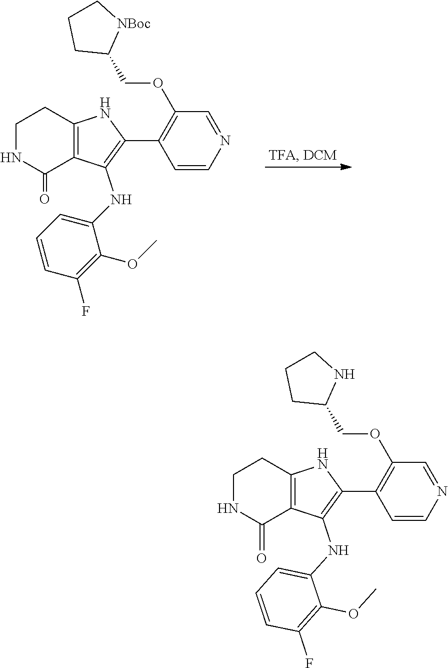 [SMILES: COc1c(F)cccc1Nc1c(-c2ccncc2OC[C@@H]2CCCN2C(=O)OC(C)(C)C)[nH]c2c1C(=O)NCC2 ; COc1c(F)cccc1Nc1c(-c2ccncc2OC[C@@H]2CCCN2)[nH]c2c1C(=O)NCC2]

A mixture of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (290 mg, 0.53 mmol, 1.00 equiv) and TFA (1 mL) in DCM (3 mL) was stirred for 1 h at room temperature under nitrogen atmosphere. The resulting mixture was concentrated under reduced pressure to afford 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (540 mg, crude) as a red oil.

LC-MS: (M+H)⁺ found 452.20

### 99.3. Synthesis of 3-[(3-fluoro-2-methoxyphenyl)amino]-2-(3-{[(2S)-1-(2-fluoroprop-2-enoyl)pyrrolidin-2-yl]methoxy}pyridin-4-yl)-1H,5H,6H,7H-pyrrolo[3,2-c]pyridin-4-one

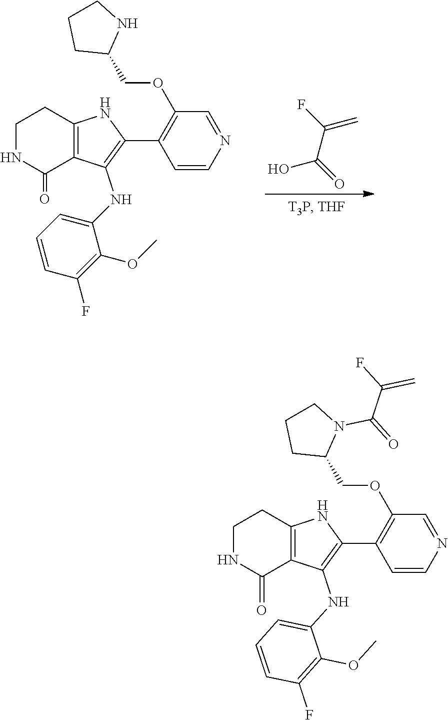

A solution of 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (60 mg, 0.13 mmol, 1.00 equiv) in THF (1.5 mL). The mixture was basified to pH 8 with DIEA, was added 2-fluoroprop-2-enoic acid (18 mg, 0.20 mmol, 1.50 equiv) at 0° C. under nitrogen atmosphere followed by the addition of T₃P (169 mg, 0.26 mmol, 2.00 equiv, 50% in EA) dropwise at 0° C. The resulting mixture was stirred for 1 h at room temperature under nitrogen atmosphere. The resulting mixture was extracted with EtOAc (3×10 mL). The combined organic layers were washed with brine (1×10 mL), The resulting mixture was washed with 1×10 mL of NaHCO₃(aq). dried over anhydrous Na₂SO₄. After filtration, the filtrate was concentrated under reduced pressure. The crude product (60 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 23% B to 53% B in 10 min; Wave Length: 254 nm; RT1(min): 9.5; Number Of Runs: 0) to afford 3-[(3-fluoro-2-methoxyphenyl)amino]-2-(3-{[(2S)-1-(2-fluoroprop-2-enoyl)pyrrolidin-2-yl]methoxy}pyridin-4-yl)-1H,5H,6H,7H-pyrrolo[3,2-c]pyridin-4-one (27.6 mg, 39.55%) as a off-white solid.

LC-MS: (M+H)⁺ found 524.10.

¹H NMR (400 MHz, DMSO-d6) δ 11.25 (s, 1H), 8.41 (s, 1H), 8.02 (d, 1H), 7.40 (s, 1H), 7.33 (d, 1H), 7.10 (s, 1H), 6.61-6.49 (m, 1H), 6.47 (t, 1H), 5.99 (d, 1H), 5.45-5.31 (m, 2H), 4.63 (s, 1H), 4.37-4.34 (m, 1H), 4.25-4.21 (m, 1H), 3.89 (s, 3H), 3.69 (s, 1H), 3.59 (s, 1H), 3.41-3.39 (m, 2H), 2.89 (t, 2H), 2.07-1.82 (m, 4H).

### Example 100. 2-(3-{[(2S)-1-(but-2-ynoyl)pyrrolidin-2-yl]methoxy}pyridin-4-yl)-3-[(3-fluoro-2-methoxyphenyl)amino]-1H,5H,6H,7H-pyrrolo[3,2-c]pyridin-4-one (compound 282)

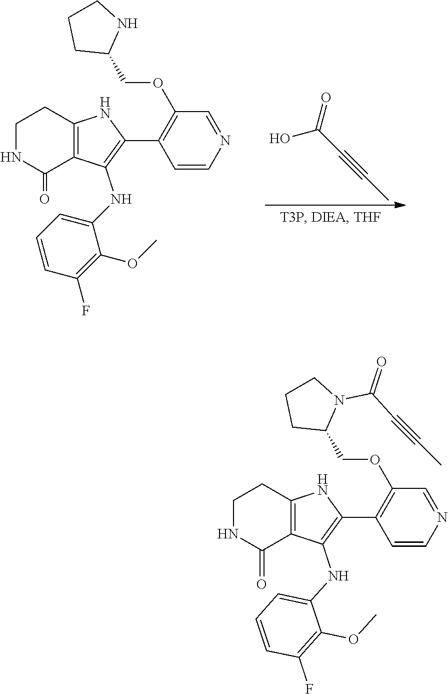

A solution of 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (60 mg, 0.13 mmol, 1.00 equiv) in THF (1.5 mL). The mixture was basified to pH 8 with DIEA, was added 2-butynoic acid (17 mg, 0.20 mmol, 1.50 equiv) over 2 min at 0° C. under nitrogen atmosphere followed by the addition of T₃P (169 mg, 0.27 mmol, 2.00 equiv, 50% in EA) dropwise at 0° C. The resulting mixture was stirred for 1 h at room temperature under nitrogen atmosphere. The resulting mixture was extracted with EtOAc (3×10 mL). The combined organic layers were washed with brine (1×10 mL). The residue was washed with NaHCO₃(aq) (1×10 mL) dried over anhydrous Na₂SO₄. After filtration, the filtrate was concentrated under reduced pressure. The crude product (60 mg) was purified by Prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 12% B to 42% B in 7 min, 42% B; Wave Length: 254 nm; RT1(min): 5.83; Number Of Runs: 0) to afford 2-(3-{[(2S)-1-(but-2-ynoyl)pyrrolidin-2-yl]methoxy}pyridin-4-yl)-3-[(3-fluoro-2-methoxyphenyl)amino]-1H,5H,6H,7H-pyrrolo[3,2-c]pyridin-4-one (29.1 mg, 42.10%) as a yellow solid.

LC-MS: (M+H)⁺ found 518.5.

¹H NMR (300 MHz, Chloroform-d) δ 11.37 (s, 1H), 8.29 (s, 1H), 7.90 (d, 1H), 7.77 (s, 1H), 7.53 (d, 1H), 6.67-6.45 (m, 2H), 6.02-5.99 (m, 1H), 5.59 (s, 1H), 4.91-4.90 (m, 1H), 4.26-4.20 (m, 1H), 4.16-4.07 (m, 4H), 4.03-4.01 (m, 1H), 3.74-3.70 (m, 1H), 3.64-3.61 (m, 2H), 3.24-3.15 (m, 2H), 2.32-1.99 (m, 2H), 2.04 (s, 3H), 1.95 (s, 1H), 1.92-1.80 (m, 1H).

### Example 101. 3-[(3-fluoro-2-methoxyphenyl)amino]-2-(3-{[(2S)-1-[(2E)-4-(morpholin-4-yl)but-2-enoyl]azetidin-2-yl]methoxy}pyridin-4-yl)-1H,5H,6H,7H-pyrrolo[3,2-c]pyridin-4-one (compound 297)

### 101.1. Synthesis of 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one

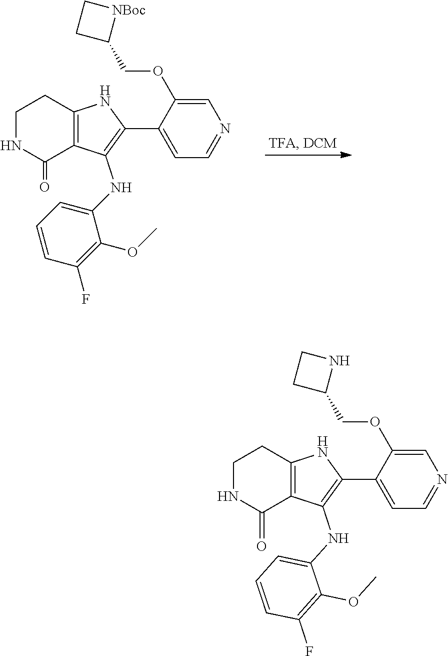 [SMILES: COc1c(F)cccc1Nc1c(-c2ccncc2OC[C@@H]2CCN2C(=O)OC(C)(C)C)[nH]c2c1C(=O)NCC2 ; COc1c(F)cccc1Nc1c(-c2ccncc2OC[C@@H]2CCN2)[nH]c2c1C(=O)NCC2]

To a stirred solution of tert-butyl 2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (150 mg, 0.28 mmol, 1.00 equiv) in DCM (2 mL) were added TFA (1 mL) dropwise at room temperature under nitrogen atmosphere. The resulting mixture was stirred for 1 h at room temperature under nitrogen atmosphere. The resulting mixture was concentrated under vacuum. The residue was dissolved in CH₂Cl₂ (5 mL). The mixture was acidified to pH 7 with saturated NaHCO₃(aq.). The resulting mixture was extracted with CH₂Cl₂:MeOH (3×10 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one (100 mg, 81.92%) as a light yellow solid. LC-MS: (M+H)⁺ found: 438.10

### 101.2. Synthesis of (S,E)-2-(3-((1-(4-bromobut-2-enoyl)azetidin-2-yl)methoxy)pyridin-4-yl)-3-((3-fluoro-2-methoxyphenyl)amino)-1,5,6,7-tetrahydro-4H-pyrrolo[3,2-c]pyridin-4-one

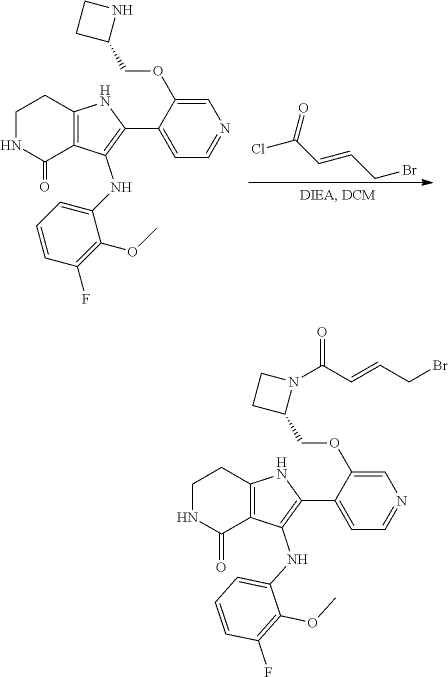

To a stirred solution of 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one (90 mg, 0.21 mmol, 1.00 equiv) and DIEA (80 mg, 0.62 mmol, 3.00 equiv) in DCM (5 mL) were added (2E)-4-bromobut-2-enoyl chloride (46 mg, 0.25 mmol, 1.20 equiv) at 0° C. under nitrogen atmosphere. The resulting mixture was stirred for 0.5h at 0° C. under nitrogen atmosphere. The resulting mixture was used in the next step directly without further purification.

LC-MS: (M+H)⁺ found: 584

### 101.3. Synthesis of 3-[(3-fluoro-2-methoxyphenyl)amino]-2-(3-{[(2S)-1-[(2E)-4-(morpholin-4-yl)but-2-enoyl]azetidin-2-yl]methoxy}pyridin-4-yl)-1H,5H,6H,7H-pyrrolo[3,2-c]pyridin-4-one

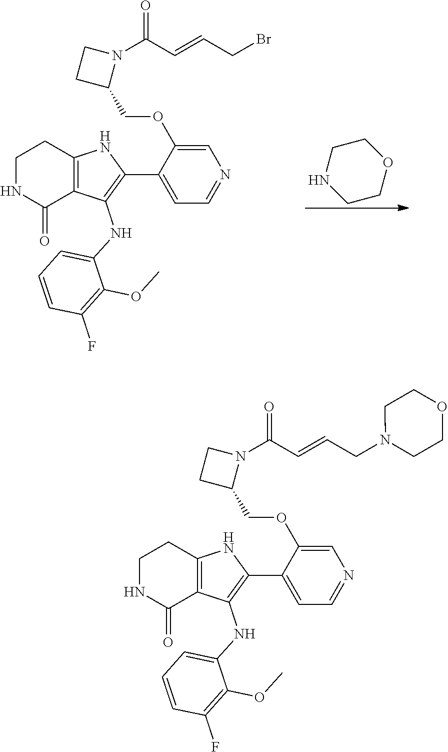 [SMILES: COc1c(F)cccc1Nc1c(-c2ccncc2OC[C@@H]2CCN2C(=O)/C=C/CBr)[nH]c2c1C(=O)NCC2 ; C1COCCN1 ; COc1c(F)cccc1Nc1c(-c2ccncc2OC[C@@H]2CCN2C(=O)/C=C/CN2CCOCC2)[nH]c2c1C(=O)NCC2]

To the last step resulting mixture was added morpholine (54 mg, 0.62 mmol, 3.00 equiv) dropwise at 0° C. The resulting mixture was stirred for additional 48 h at room temperature. The resulting mixture was extracted with CH₂Cl₂:MeOH (3×10 mL). The combined organic layers were dried over anhydrous Na₂SO₄. After filtration, the filtrate was concentrated under reduced pressure and dissolved in DMSO. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 18% B to 38% B in 10 min, 38% B; Wave Length: 254/220 nm; RT1(min): 8.85; Number Of Runs: 0) to afford 3-[(3-fluoro-2-methoxyphenyl)amino]-2-(3-{[(2S)-1-[(2E)-4-(morpholin-4-yl)but-2-enoyl]azetidin-2-yl]methoxy}pyridin-4-yl)-1H,5H,6H,7H-pyrrolo[3,2-c]pyridin-4-one (53.2 mg, 43.78%) as a off-white solid.

LC-MS: (M+H)⁺ found 591.15.

¹H NMR (400 MHz, Chloroform-d) δ 11.79 (s, 1H), 8.25 (s, 1H), 8.01 (d, 1H), 7.61 (s, 1H), 7.53 (d, 1H), 6.94-6.87 (m, 1H), 6.60-6.55 (m, 1H), 6.48-6.43 (m, 1H), 6.19-6.04 (m, 2H), 5.22 (s, 1H), 5.04 (q, 1H), 4.53 (t, 1H), 4.42-4.19 (m, 3H), 4.13 (d, 3H), 3.76 (t, 4H), 3.62-3.57 (m, 2H), 3.26-3.05 (m, 4H), 2.70-2.61 (m, 1H), 2.51-2.49 (m, 4H), 2.21-2.13 (m, 1H).

### Example 102. 3-[(3-fluoro-2-methoxyphenyl)amino]-2-(3-{[(2S)-1-[(2E)-4-(morpholin-4-yl)but-2-enoyl]pyrrolidin-2-yl]methoxy}pyridin-4-yl)-1H,5H,6H,7H-pyrrolo[3,2-c]pyridin-4-one (compound 300)

### 102.1. Synthesis of 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one

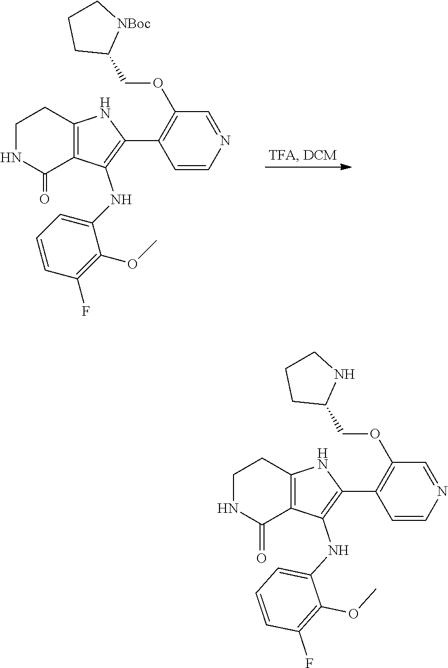 [SMILES: COc1c(F)cccc1Nc1c(-c2ccncc2OC[C@@H]2CCCN2C(=O)OC(C)(C)C)[nH]c2c1C(=O)NCC2 ; COc1c(F)cccc1Nc1c(-c2ccncc2OC[C@@H]2CCCN2)[nH]c2c1C(=O)NCC2]

To a stirred solution of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (150 mg, 0.27 mmol, 1.00 equiv) in DCM (2 mL) were added TFA (1 mL) dropwise at room temperature under nitrogen atmosphere. The resulting mixture was stirred for 1 h at room temperature under nitrogen atmosphere. The resulting mixture was concentrated under vacuum. The residue was dissolved in CH₂Cl₂ (5 mL). The mixture was acidified to pH 7 with saturated NaHCO₃(aq.). The resulting mixture was extracted with CH₂Cl₂:MeOH (3×10 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (100 mg, 81.45%) as a light yellow solid.

LC-MS: (M+H)⁺ found 452.10

### 102.2. Synthesis of (S,E)-2-(3-((1-(4-bromobut-2-enoyl)pyrrolidin-2-yl)methoxy)pyridin-4-yl)-3-((3-fluoro-2-methoxyphenyl)amino)-1,5,6,7-tetrahydro-4H-pyrrolo[3,2-c]pyridin-4-one

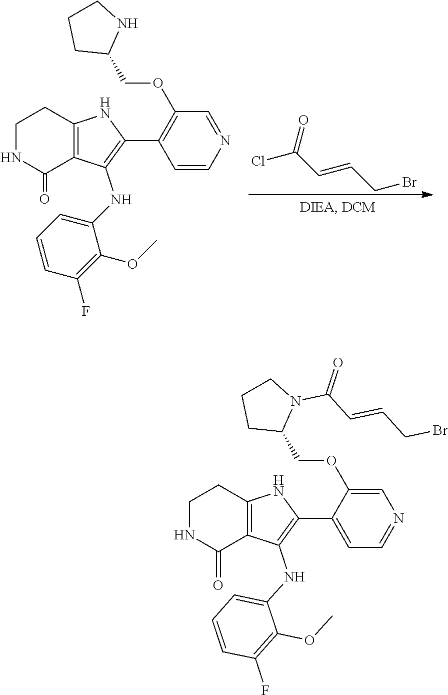

To a stirred solution of 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (100 mg, 0.22 mmol, 1.00 equiv) and DIEA (86 mg, 0.66 mmol, 3.00 equiv) in DCM (5 mL) were added (2E)-4-bromobut-2-enoyl chloride (49 mg, 0.26 mmol, 1.20 equiv) at 0° C. under nitrogen atmosphere. The resulting mixture was stirred for 0.5 h at 0° C. under nitrogen atmosphere. The resulting mixture was used in the next step directly without further purification.

LC-MS: (M+H)⁺ found: 554.10

### 102.3. Synthesis of 3-[(3-fluoro-2-methoxyphenyl)amino]-2-(3-{[(2S)-1-[(2E)-4-(morpholin-4-yl)but-2-enoyl]pyrrolidin-2-yl]methoxy}pyridin-4-yl)-1H,5H,6H,7H-pyrrolo[3,2-c]pyridin-4-one

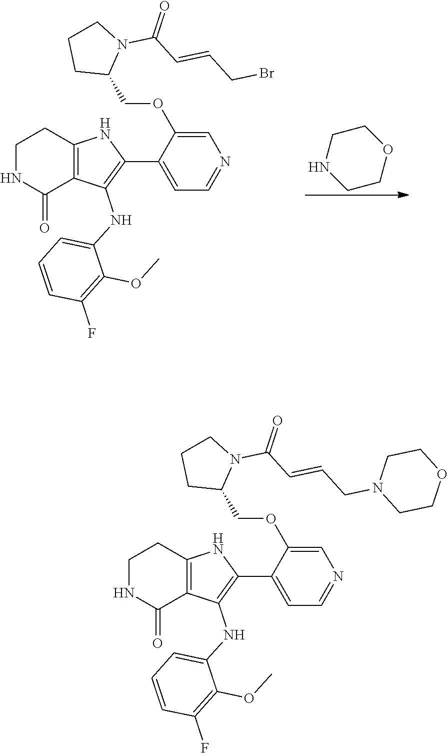 [SMILES: COc1c(F)cccc1Nc1c(-c2ccncc2OC[C@@H]2CCCN2C(=O)/C=C/CBr)[nH]c2c1C(=O)NCC2 ; C1COCCN1 ; COc1c(F)cccc1Nc1c(-c2ccncc2OC[C@@H]2CCCN2C(=O)/C=C/CN2CCOCC2)[nH]c2c1C(=O)NCC2]

To the last step resulting mixture was added morpholine (58 mg, 0.66 mmol, 3.00 equiv) dropwise at 0° C. The resulting mixture was stirred for additional 36 h at room temperature. The resulting mixture was extracted with CH₂Cl₂:MeOH (3×20 mL). The combined organic layers were dried over anhydrous Na₂SO₄. After filtration, the filtrate was concentrated under reduced pressure and dissolved in DMSO. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 24% B to 39% B in 10 min, 39% B; Wave Length: 254/220 nm; RT1(min): 7.53; Number Of Runs: 0) to afford 3-[(3-fluoro-2-methoxyphenyl)amino]-2-(3-{[(2S)-1-[(2E)-4-(morpholin-4-yl)but-2-enoyl]pyrrolidin-2-yl]methoxy}pyridin-4-yl)-1H,5H,6H,7H-pyrrolo[3,2-c]pyridin-4-one (40.4 mg, 30.17%) as a off-white solid.

LC-MS: (M+H)⁺ found 605.15.

¹H NMR (400 MHz, Chloroform-d) δ 11.56 (s, 1H), 8.20 (brs, 1H), 7.96 (brs, 1H), 7.65 (s, 1H), 7.56 (brs, 1H), 6.96-6.89 (m, 1H), 6.62-6.56 (m, 1H), 6.50-6.40 (m, 2H), 6.06-6.04 (m, 1H), 5.21 (s, 1H), 5.03 (t, 1H), 4.28 (t, 1H), 4.21-4.06 (m, 4H), 3.77-3.72 (m, 6H), 3.64-3.57 (m, 2H), 3.29-3.14 (m, 4H), 2.49 (t, 4H), 2.24-2.09 (m, 3H), 1.88-1.85 (m, 1H).

### Example 103. 3-[(3-fluoro-2-methoxyphenyl)amino]-2-(3-{[(2R)-1-[(2E)-4-(morpholin-4-yl)but-2-enoyl]pyrrolidin-2-yl]methoxy}pyridin-4-yl)-1H,5H,6H,7H-pyrrolo[3,2-c]pyridin-4-one (compound 299)

### 103.1. Synthesis of 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one

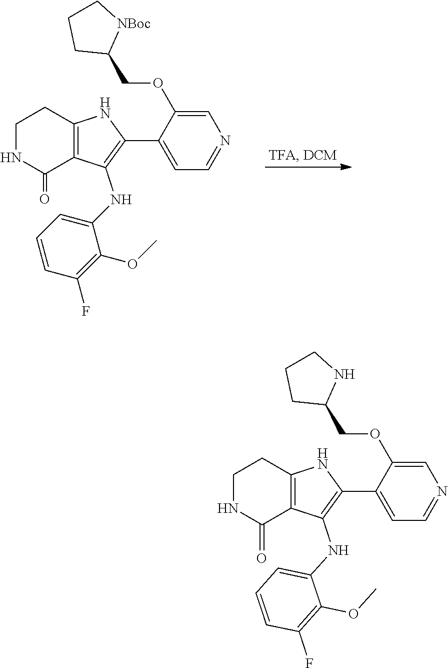 [SMILES: COc1c(F)cccc1Nc1c(-c2ccncc2OC[C@H]2CCCN2C(=O)OC(C)(C)C)[nH]c2c1C(=O)NCC2 ; COc1c(F)cccc1Nc1c(-c2ccncc2OC[C@H]2CCCN2)[nH]c2c1C(=O)NCC2]

To a stirred solution of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (150 mg, 0.27 mmol, 1.00 equiv) in DCM (2 mL) were added TFA (1 mL) dropwise at room temperature under nitrogen atmosphere. The resulting mixture was stirred for 1 h at room temperature under nitrogen atmosphere. The resulting mixture was concentrated under vacuum. The residue was dissolved in CH₂Cl₂ (5 mL). The mixture was acidified to pH 7 with saturated NaHCO₃(aq.). The resulting mixture was extracted with CH₂Cl₂:MeOH (3×10 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one (100 mg, 81.45%) as a light yellow solid.

LC-MS: (M+H)⁺ found 452.10

### 103.2. Synthesis of (R,E)-2-(3-((1-(4-bromobut-2-enoyl)pyrrolidin-2-yl)methoxy)pyridin-4-yl)-3-((3-fluoro-2-methoxyphenyl)amino)-1,5,6,7-tetrahydro-4H-pyrrolo[3,2-c]pyridin-4-one

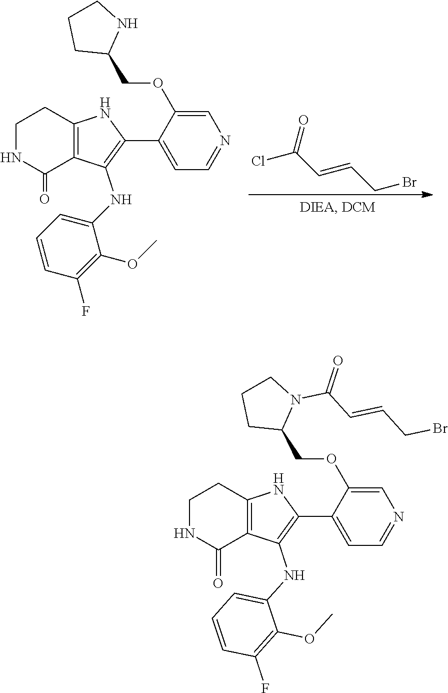

To a stirred solution of 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one (90 mg, 0.20 mmol, 1.00 equiv) and DIEA (78 mg, 0.60 mmol, 3.00 equiv) in DCM (5 mL) were added (2E)-4-bromobut-2-enoyl chloride (44 mg, 0.24 mmol, 1.20 equiv) at 0° C. under nitrogen atmosphere. The resulting mixture was stirred for 0.5h at 0° C. under nitrogen atmosphere The resulting mixture was used in the next step directly without further purification.

LC-MS: (M+H)⁺ found 554.10

### 103.3. Synthesis of 3-[(3-fluoro-2-methoxyphenyl)amino]-2-(3-{[(2R)-1-[(2E)-4-(morpholin-4-yl)but-2-enoyl]pyrrolidin-2-yl]methoxy}pyridin-4-yl)-1H,5H,6H,7H-pyrrolo[3,2-c]pyridin-4-one

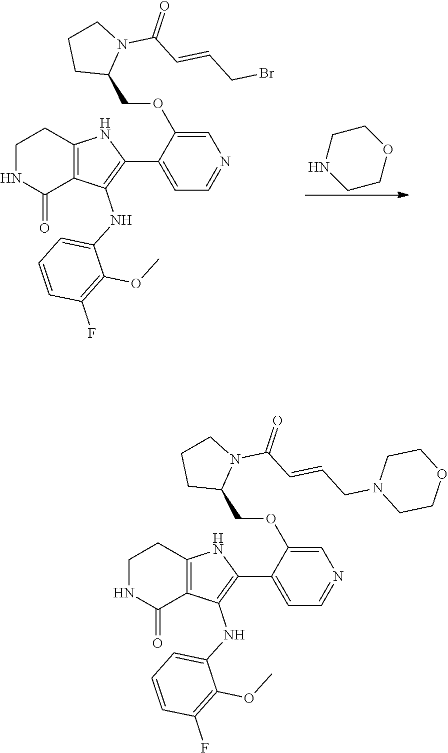 [SMILES: COc1c(F)cccc1Nc1c(-c2ccncc2OC[C@H]2CCCN2C(=O)/C=C/CBr)[nH]c2c1C(=O)NCC2 ; C1COCCN1 ; COc1c(F)cccc1Nc1c(-c2ccncc2OC[C@H]2CCCN2C(=O)/C=C/CN2CCOCC2)[nH]c2c1C(=O)NCC2]

To the last step resulting mixture was added morpholine (52 mg, 0.59 mmol, 3 equiv) dropwise at 0° C. The resulting mixture was stirred for additional 48 h at room temperature. The resulting mixture was extracted with CH₂Cl₂:MeOH (3×50 mL). The combined organic layers were dried over anhydrous Na₂SO₄. After filtration, the filtrate was concentrated under reduced pressure and dissolved in DMSO. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 20% B to 40% B in 9 min, 40% B; Wave Length: 254/220 nm; RT1(min): 8.85; Number Of Runs: 0) to afford 3-[(3-fluoro-2-methoxyphenyl)amino]-2-(3-{[(2R)-1-[(2E)-4-(morpholin-4-yl)but-2-enoyl]pyrrolidin-2-yl]methoxy}pyridin-4-yl)-1H,5H,6H,7H-pyrrolo[3,2-c]pyridin-4-one (51.2 mg, 42.48%) as a off-white solid.

LC-MS: (M+H)⁺ found 605.15.

¹H NMR (400 MHz, Chloroform-d) δ 11.54 (s, 1H), 8.23 (s, 1H), 8.00 (d, 1H), 7.63-7.49 (m, 2H), 6.94-6.88 (m, 1H), 6.61-6.55 (m, 1H), 6.49-6.39 (m, 2H), 6.12-6.05 (m, 1H), 5.23 (s, 1H), 5.06 (t, 1H), 4.28 (t, 1H), 4.19-4.05 (m, 4H), 3.76-3.71 (m, 6H), 3.67-3.54 (m, 2H), 3.27-3.07 (m, 4H), 2.50 (t, 4H), 2.21-2.16 (m, 3H), 1.92-1.83 (m, 1H).

### Example 104. 2-(3-{[(2R)-1-[4-(dimethylamino)but-2-ynoyl]azetidin-2-yl]methoxy}pyridin-4-yl)-3-[(3-fluoro-2-methoxyphenyl)amino]-1H,5H,6H,7H-pyrrolo[3,2-c]pyridin-4-one (compound 326)

### 104.1. Synthesis of (R)-2-(3-(azetidin-2-ylmethoxy)pyridin-4-yl)-3-((3-fluoro-2-methoxyphenyl)amino)-1,5,6,7-tetrahydro-4H-pyrrolo[3,2-c]pyridin-4-one

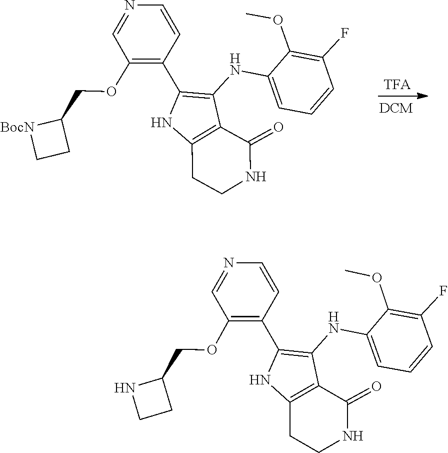 [SMILES: COc1c(F)cccc1Nc1c(-c2ccncc2OC[C@H]2CCN2C(=O)OC(C)(C)C)[nH]c2c1C(=O)NCC2 ; COc1c(F)cccc1Nc1c(-c2ccncc2OC[C@H]2CCN2)[nH]c2c1C(=O)NCC2]

To a stirred solution of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (200 mg, 0.19 mmol, 1.00 equiv) in DCM (1 mL) was added TFA (0.2 mL). The resulting mixture was stirred for 3 h at room temperature under nitrogen atmosphere. The resulting mixture was concentrated under reduced pressure. The crude product was used in the next step directly without further purification.

LC-MS: (M+H)⁺ found: 438.05

### 104.2. Synthesis of 2-(3-{[(2R)-1-[4-(dimethylamino)but-2-ynoyl]azetidin-2-yl]methoxy}pyridin-4-yl)-3-[(3-fluoro-2-methoxyphenyl)amino]-1H,5H,6H,7H-pyrrolo[3,2-c]pyridin-4-one

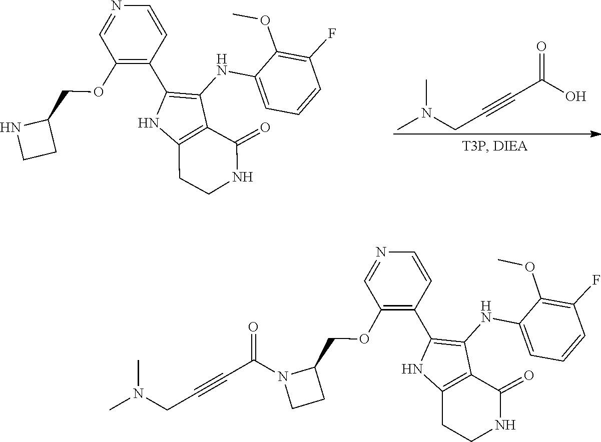

To a stirred solution of 2-{3-[(2R)-azetidin-2-ylmethoxy]pyridin-4-yl}-3-[(3-fluoro-2-methoxyphenyl)amino]-1H,5H,6H,7H-pyrrolo[3,2-c]pyridin-4-one (110 mg, 0.251 mmol, 1 equiv) and 4-(dimethylamino)but-2-ynoic acid (95.91 mg, 0.753 mmol, 3 equiv) in THF (3 mL) was added DIEA (162.49 mg, 1.255 mmol, 5 equiv) dropwise at 0° C. under nitrogen atmosphere. To the above mixture was added T₃P (480.03 mg, 0.753 mmol, 3 equiv, 50% in EA) dropwise at 0° C. The resulting mixture was stirred for additional 2 h at room temperature. The reaction was quenched with sat. NaHCO₃(aq.) at 0° C. The resulting mixture was extracted with EtOAc (3×10 mL). The combined organic layers were washed with brine (1×10 mL), dried over anhydrous Na₂SO₄. After filtration, the filtrate was concentrated under reduced pressure. The crude product (90 mg) was purified by Prep-HPLC with the following conditions (Column: XSelect CSH Fluoro Phenyl, 30*150 mm, 5 μm; Mobile Phase A: Water (10 mmol/L NH₄HCO₃), Mobile Phase B: ACN; Flow rate: 60 mL/min; Gradient: 30% B to 60% B in 7 min; Wave Length: 254 nm; RT1(min): 6.5; Number Of Runs: 0) to afford 2-(3-{[(2R)-1-[4-(dimethylamino)but-2-ynoyl]azetidin-2-yl]methoxy}pyridin-4-yl)-3-[(3-fluoro-2-methoxyphenyl)amino]-1H,5H,6H,7H-pyrrolo[3,2-c]pyridin-4-one (12.2 mg, 8.83%) as a light yellow solid. LC-MS: (M+H)⁺ found: 547.15.

¹H NMR (400 MHz, Chloroform-d) δ 11.43 (s, 1H), 8.26 (s, 1H), 7.96 (d, 1H), 7.71 (s, 1H), 7.49 (d, 1H), 6.61-6.55 (m, 1H), 6.51-6.46 (m, 1H), 6.10-5.99 (m, 1H), 5.26 (s, 1H), 4.98 (q, 1H), 4.53 (t, 1H), 4.38-4.23 (m, 3H), 4.10 (d, 3H), 3.61-3.57 (m, 2H), 3.51 (s, 2H), 3.24-3.12 (m, 1H), 3.10-3.03 (m, 1H), 2.71-2.61 (m, 1H), 2.19-2.15 (m, 1H).

### Example 105. 2-(3-{[(2R)-1-(but-2-ynoyl)azetidin-2-yl]methoxy}pyridin-4-yl)-3-[(4-chlorophenyl)amino]-1H,5H,6H,7H-pyrrolo[3,2-c]pyridin-4-one (compound 353)

### 105.1. Synthesis of tert-butyl (2R)-2-{[(4-{3-[(4-chlorophenyl)amino]-4-oxo-1H,5H,6H,7H-pyrrolo[3,2-c]pyridin-2-yl}pyridin-3-yl)oxy]methyl}azetidine-1-carboxylate

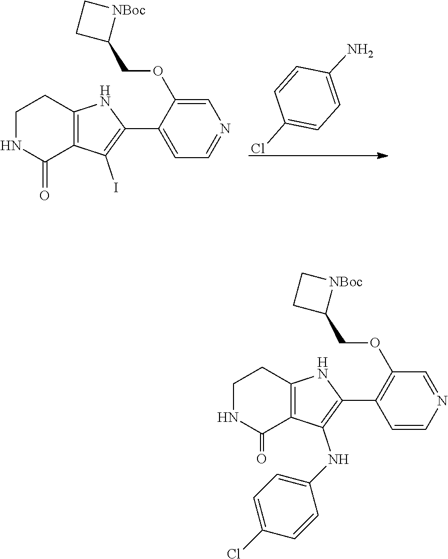

To a stirred solution of tert-butyl (2S)-2-{[(4-{3-iodo-4-oxo-1H,5H,6H,7H-pyrrolo[3,2-c]pyridin-2-yl}pyridin-3-yl)oxy]methyl}azetidine-1-carboxylate (300 mg, 0.57 mmol, 1.00 equiv) and 4-chloroaniline (73 mg, 0.57 mmol, 1.00 equiv) in DMF (5 mL) was added Cs₂CO₃ (559 mg, 1.71 mmol, 3.00 equiv) and EPhos Pd G4 (78.83 mg, 0.08 mmol, 0.15 equiv). The resulting mixture was stirred for 3 h at 50° C. under argon atmosphere. The resulting mixture was concentrated under reduced pressure. The residue was purified by silica gel column chromatography, eluted with CH₂Cl₂/MeOH (10:1) to afford tert-butyl (2R)-2-{[(4-{3-[(4-chlorophenyl)amino]-4-oxo-1H,5H,6H,7H-pyrrolo[3,2-c]pyridin-2-yl}pyridin-3-yl)oxy]methyl}azetidine-1-carboxylate (180 mg, 60.04%) as a yellow solid.

LC-MS: (M+H)⁺ found: 524.10

### 105.2. Synthesis of (R)-2-(3-(azetidin-2-ylmethoxy)pyridin-4-yl)-3-((4-chlorophenyl)amino)-1,5,6,7-tetrahydro-4H-pyrrolo[3,2-c]pyridin-4-one

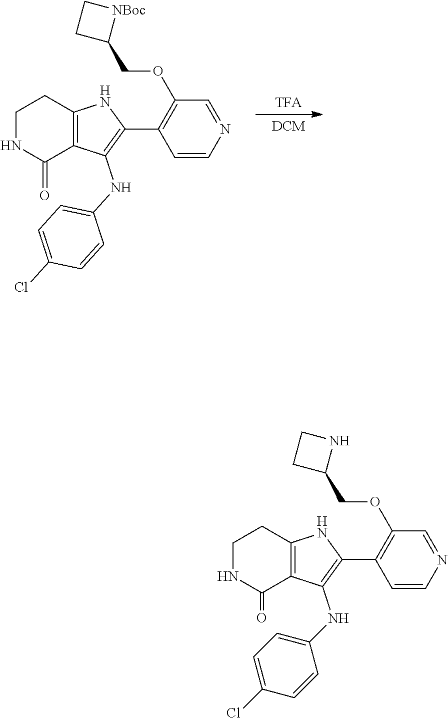 [SMILES: CC(C)(C)OC(=O)N1CC[C@@H]1COc1cnccc1-c1[nH]c2c(c1Nc1ccc(Cl)cc1)C(=O)NCC2 ; O=C1NCCc2[nH]c(-c3ccncc3OC[C@H]3CCN3)c(Nc3ccc(Cl)cc3)c21]

To a stirred solution of tert-butyl (2R)-2-{[(4-{3-[(4-chlorophenyl)amino]-4-oxo-1H,5H,6H,7H-pyrrolo[3,2-c]pyridin-2-yl}pyridin-3-yl)oxy]methyl}azetidine-1-carboxylate (100 mg, 0.19 mmol, 1.00 equiv) in DCM (2 mL) was added TFA (0.4 mL). The resulting mixture was stirred for 3 h at room temperature under nitrogen atmosphere. The resulting mixture was concentrated under reduced pressure. The crude product was used in the next step directly without further purification.

LC-MS: (M+H)⁺ found: 424.00

### 105.3. Synthesis of 2-(3-{[(2R)-1-(but-2-ynoyl)azetidin-2-yl]methoxy}pyridin-4-yl)-3-[(4-chlorophenyl)amino]-1H,5H,6H,7H-pyrrolo[3,2-c]pyridin-4-one

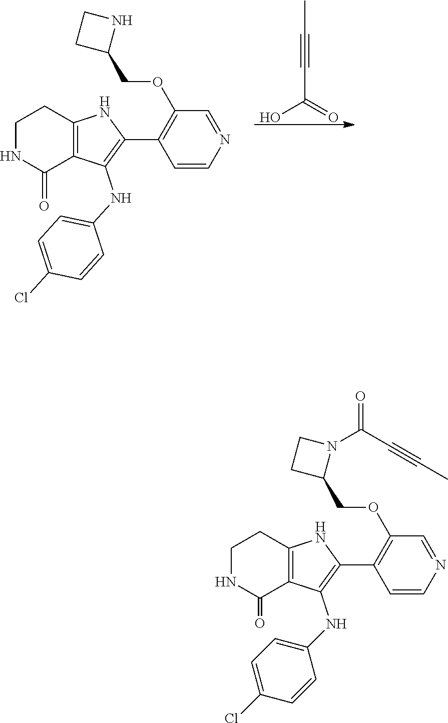

To a stirred solution of 2-{3-[(2R)-azetidin-2-ylmethoxy]pyridin-4-yl}-3-[(4-chlorophenyl)amino]-1H,5H,6H,7H-pyrrolo[3,2-c]pyridin-4-one (90 mg, 0.21 mmol, 1.00 equiv) and 2-butynoic acid (27 mg, 0.32 mmol, 1.50 equiv) in THF (2 mL) was added DIEA (137 mg, 1.06 mmol, 5.00 equiv) to adjust PH to 8 at 0° C. To the above mixture was added T₃P (270 mg, 0.42 mmol, 2.00 equiv, 50% in EA) dropwise at 0° C. The resulting mixture was stirred for additional 2 h at room temperature. The reaction was quenched with sat. NaHCO₃(aq.) at 0° C. The resulting mixture was extracted with EtOAc (3×10 mL). The combined organic layers were washed with NaHCO₃(sat.) (2×10 mL), dried over anhydrous Na₂SO₄. After filtration, the filtrate was concentrated under reduced pressure. The crude product (90 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 25% B to 48% B in 9 min, 48% B; Wave Length: 254/220 nm; RT1(min): 7.53; Number Of Runs: 0) to afford 2-(3-{[(2R)-1-(but-2-ynoyl)azetidin-2-yl]methoxy}pyridin-4-yl)-3-[(4-chlorophenyl)amino]-1H,5H,6H,7H-pyrrolo[3,2-c]pyridin-4-one (24 mg, 23.00%) as a yellow solid.

LC-MS: (M+H)⁺ found: 490.05.

¹H NMR (400 MHz, Chloroform-d) δ 11.42 (s, 1H), 8.23 (s, 1H), 7.92 (d, 1H), 7.48 (s, 1H), 7.38 (d, 1H), 7.07-6.96 (m, 2H), 6.68-6.53 (m, 2H), 5.26 (s, 1H), 4.95 (q, 1H), 4.50 (t, 1H), 4.26-4.25 (m, 3H), 3.59-3.57 (m, 2H), 3.26-3.00 (m, 2H), 2.69-2.58 (m, 1H), 2.17-2.11 (m, 1H), 2.04 (s, 3H).

### Example 106. 2-(3-{[(2R)-1-(but-2-ynoyl)azetidin-2-yl]methoxy}pyridin-4-yl)-3-[(3-chloro-2-ethylphenyl)amino]-1H,5H,6H,7H-pyrrolo[3,2-c]pyridin-4-one (compound 348)

### 106.1. Synthesis of tert-butyl (2R)-2-{[(4-{3-[(3-chloro-2-ethylphenyl)amino]-4-oxo-1H,5H,6H,7H-pyrrolo[3,2-c]pyridin-2-yl}pyridin-3-yl)oxy]methyl}azetidine-1-carboxylate

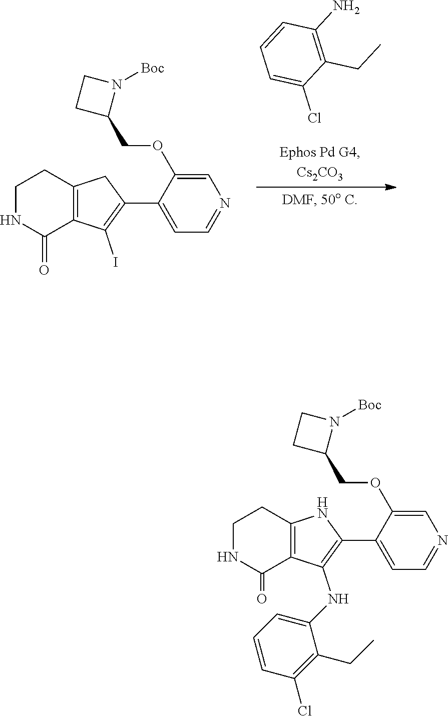

To a stirred solution of tert-butyl (2R)-2-{[(4-{7-iodo-1-oxo-2H,3H,4H,5H-cyclopenta[c]pyridin-6-yl}pyridin-3-yl)oxy]methyl}azetidine-1-carboxylate (300 mg, 0.57 mmol, 1.00 equiv) and 3-chloro-2-ethylaniline (89 mg, 0.57 mmol, 1.00 equiv) in DMF (3 mL) were added EPhos Pd G4 (53 mg, 0.06 mmol, 0.10 equiv) and Cs₂CO₃ (561 mg, 1.72 mmol, 3.00 equiv) in portions at room temperature under argon atmosphere. The resulting mixture was stirred for 6 h at 50° C. under argon atmosphere. The resulting mixture was concentrated under reduced pressure. The residue was purified by silica gel column chromatography, eluted with CH₂Cl₂/MeOH (97:3) to afford tert-butyl (2R)-2-{[(4-{3-[(3-chloro-2-ethylphenyl)amino]-4-oxo-1H,5H,6H,7H-pyrrolo[3,2-c]pyridin-2-yl}pyridin-3-yl)oxy]methyl}azetidine-1-carboxylate (210 mg, 66.36%) as a yellow solid.

LC-MS: (M+H)⁺ found: 552.15

### 106.2. Synthesis of 2-{3-[(2R)-azetidin-2-ylmethoxy]pyridin-4-yl}-3-[(3-chloro-2-ethylphenyl)amino]-1H,5H,6H,7H-pyrrolo[3,2-c]pyridin-4-one

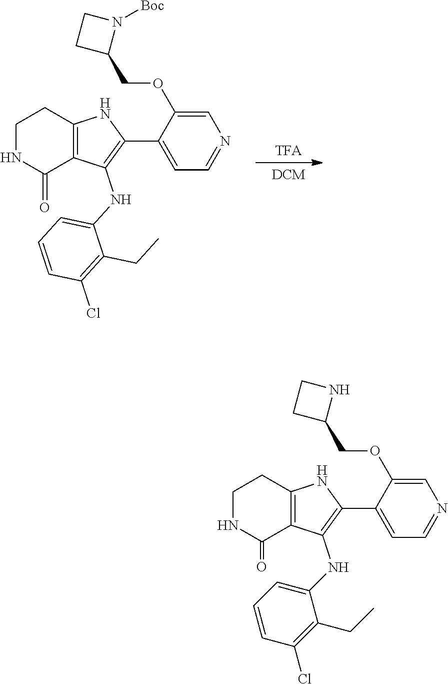 [SMILES: CCc1c(Cl)cccc1Nc1c(-c2ccncc2OC[C@H]2CCN2C(=O)OC(C)(C)C)[nH]c2c1C(=O)NCC2 ; CCc1c(Cl)cccc1Nc1c(-c2ccncc2OC[C@H]2CCN2)[nH]c2c1C(=O)NCC2]

To a stirred solution of tert-butyl (2R)-2-{[(4-{3-[(3-chloro-2-ethylphenyl)amino]-4-oxo-1H,5H,6H,7H-pyrrolo[3,2-c]pyridin-2-yl}pyridin-3-yl)oxy]methyl}azetidine-1-carboxylate (100 mg, 0.18 mmol, 1.00 equiv) in DCM (2 mL) were added TFA (1 mL) dropwise at room temperature under nitrogen atmosphere. The resulting mixture was stirred for 1 h at room temperature under nitrogen atmosphere. The resulting mixture was concentrated under reduced pressure. The crude product 2-{3-[(2R)-azetidin-2-ylmethoxy]pyridin-4-yl}-3-[(3-chloro-2-ethylphenyl)amino]-1H,5H,6H,7H-pyrrolo[3,2-c]pyridin-4-one (150 mg) was used in the next step directly without further purification.

LC-MS: (M+H)⁺ found: 452.00

### 106.3. Synthesis of 2-(3-{[(2R)-1-(but-2-ynoyl)azetidin-2-yl]methoxy}pyridin-4-yl)-3-[(3-chloro-2-ethylphenyl)amino]-1H,5H,6H,7H-pyrrolo[3,2-c]pyridin-4-one

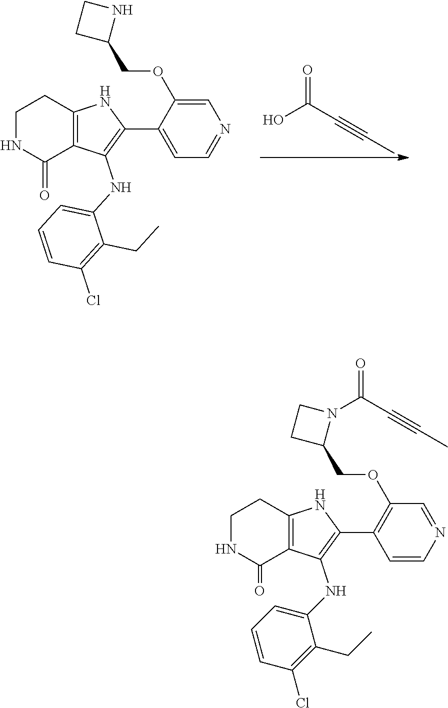

To a stirred solution of 2-{3-[(2R)-azetidin-2-ylmethoxy]pyridin-4-yl}-3-[(3-chloro-2-ethylphenyl)amino]-1H,5H,6H,7H-pyrrolo[3,2-c]pyridin-4-one (100 mg, 0.22 mmol, 1.00 equiv) in THF (4 mL) were added DIEA (143 mg, 1.11 mmol, 5.00 equiv) to basified the mixture. To the above mixture was added 2-butynoic acid (28 mg, 0.33 mmol, 1.50 equiv) and T₃P (282 mg, 0.44 mmol, 2.00 equiv, 50% in EA) dropwise. The resulting mixture was stirred for additional 1 h at room temperature. The resulting mixture was extracted with CH₂Cl₂:MeOH (3×20 mL). The combined organic layers were washed with NaHCO₃(sat.) (2×20 mL), dried over anhydrous Na₂SO₄. After filtration, the filtrate was concentrated under reduced pressure and dissolved in DMSO. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 37% B to 60% B in 9 min, 60% B; Wave Length: 254/220 nm; RT1(min): 8.85; Number Of Runs: 0) to afford 2-(3-{[(2R)-1-(but-2-ynoyl)azetidin-2-yl]methoxy}pyridin-4-yl)-3-[(3-chloro-2-ethylphenyl)amino]-1H,5H,6H,7H-pyrrolo[3,2-c]pyridin-4-one (23.5 mg, 20.50%) as a orange solid.

LC-MS: (M+H)⁺ found 518.10.

¹H NMR (400 MHz, Chloroform-d) δ 11.41 (s, 1H), 8.22 (s, 1H), 7.91 (d, 1H), 7.61 (s, 1H), 7.24 (d, 1H), 6.80-6.78 (m, 1H), 6.69 (t, 1H), 6.36-6.32 (m, 1H), 5.26 (t, 1H), 4.97 (q, 1H), 4.51 (t, 1H), 4.28-4.23 (m, 3H), 3.60-3.57 (m, 2H), 3.26-2.91 (m, 4H), 2.70-2.58 (m, 1H), 2.14-2.11 (m, 1H), 2.06 (s, 3H), 1.39 (t, 3H).

### Example 107. 3-[(2-ethyl-3-fluorophenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one (compound 364)

### 107.1. Synthesis of 2-{3-[(2R)-azetidin-2-ylmethoxy]pyridin-4-yl}-3-[(2-ethyl-3-fluorophenyl)amino]-1H,5H,6H,7H-pyrrolo[3,2-c]pyridin-4-one

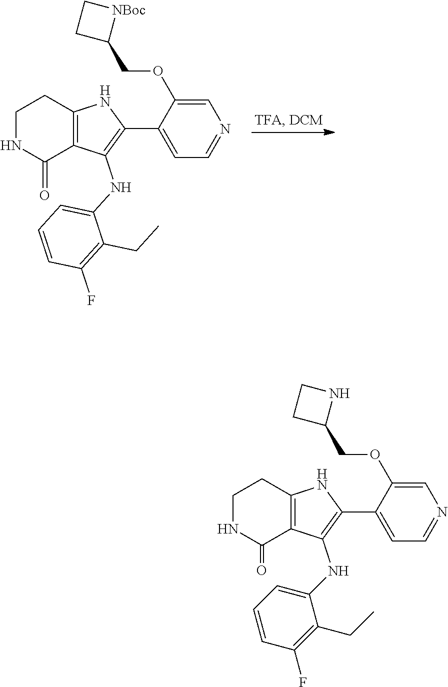 [SMILES: CCc1c(F)cccc1Nc1c(-c2ccncc2OC[C@H]2CCN2C(=O)OC(C)(C)C)[nH]c2c1C(=O)NCC2 ; CCc1c(F)cccc1Nc1c(-c2ccncc2OC[C@H]2CCN2)[nH]c2c1C(=O)NCC2]

A mixture of tert-butyl (2S)-2-{[(4-{3-[(2-ethyl-3-fluorophenyl)amino]-4-oxo-1H,5H,6H,7H-pyrrolo[3,2-c]pyridin-2-yl}pyridin-3-yl)oxy]methyl}azetidine-1-carboxylate (100 mg, 0.18 mmol, 1.00 equiv) and TFA (0.2 mL) in DCM (1 mL) was stirred for 1 h at room temperature under nitrogen atmosphere. The resulting mixture was concentrated under reduced pressure to afford 2-{3-[(2R)-azetidin-2-ylmethoxy]pyridin-4-yl}-3-[(2-ethyl-3-fluorophenyl)amino]-1H,5H,6H,7H-pyrrolo[3,2-c]pyridin-4-one (200 mg, crude) as a red oil.

LC-MS: (M+H)⁺ found 436.20

### 107.2. Synthesis of 3-[(2-ethyl-3-fluorophenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one

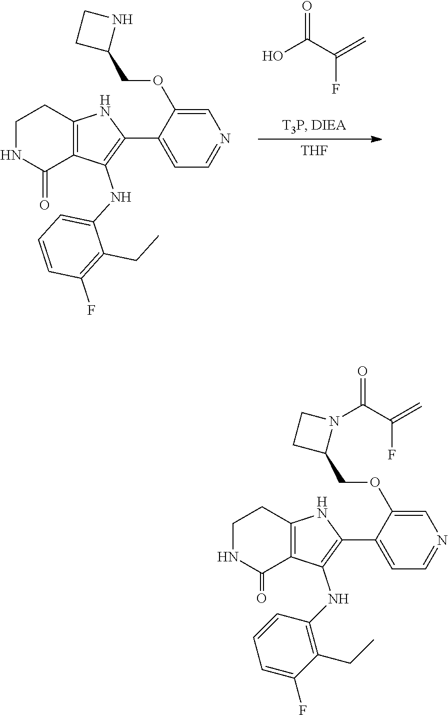

To a stirred mixture of 2-{3-[(2R)-azetidin-2-ylmethoxy]pyridin-4-yl}-3-[(2-ethyl-3-fluorophenyl)amino]-1H,5H,6H,7H-pyrrolo[3,2-c]pyridin-4-one (60 mg, 0.14 mmol, 1.00 equiv) in THF (2 mL), the mixture was basified to pH 8 with DIEA, were added 2-fluoroprop-2-enoic acid (25 mg, 0.28 mmol, 2.00 equiv) and T₃P (175 mg, 0.28 mmol, 2.00 equiv, 50% in EA) dropwise at 0° C. under nitrogen atmosphere. The resulting mixture was stirred for 3h at room temperature under nitrogen atmosphere. The resulting mixture was extracted with EtOAc (3×10 mL). The combined organic layers were washed with NaHCO₃(aq) (1×10 mL), dried over anhydrous Na₂SO₄. After filtration, the filtrate was concentrated under reduced pressure. The crude product (60 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 17% B to 47% B in 7 min; Wave Length: 254 nm; RT1(min): 6.5; Number Of Runs: 0) to afford 3-[(2-ethyl-3-fluorophenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one (25.6 mg, 35.18%) as a light yellow solid.

LC-MS: (M+H)⁺ found 508.2

¹H NMR (400 MHz, Chloroform-d) δ 11.48 (s, 1H), 8.23 (s, 1H), 7.93 (d, 1H), 7.54 (s, 1H), 7.29 (d, 1H), 6.72-6.69 (m, 1H), 6.50 (t, 1H), 6.25 (d, 1H), 5.71-5.59 (m, 1H), 5.34 (s, 1H), 5.22 (d, 1H), 5.11 (q, 1H), 4.62-4.40 (m, 3H), 4.30 (d, 1H), 3.62-3.58 (m, 2H), 3.13 (t, 2H), 2.97-2.77 (m, 2H), 2.70-2.66 (m, 1H), 2.26-2.15 (m, 1H), 1.38 (t, 3H).

### Example 108. 2-(3-{2-[(2R)-1-[(2E)-4-(dimethylamino)but-2-enoyl]-2-methylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one (compound 335)

### 108.1. Synthesis of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate

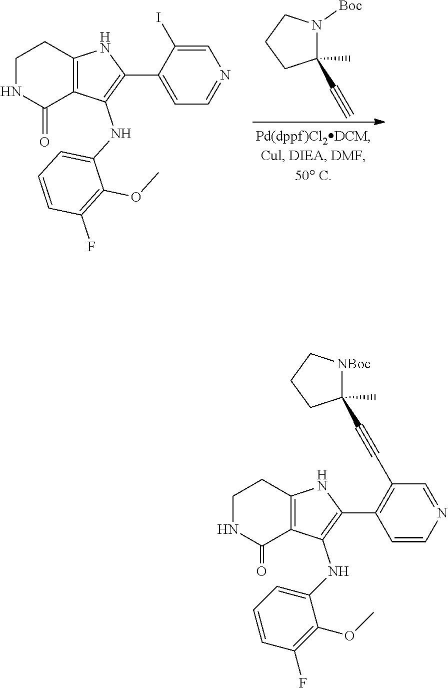 [SMILES: COc1c(F)cccc1Nc1c(-c2ccncc2I)[nH]c2c1C(=O)NCC2 ; C#C[C@@]1(C)CCCN1C(=O)OC(C)(C)C ; COc1c(F)cccc1Nc1c(-c2ccncc2C#C[C@@]2(C)CCCN2C(=O)OC(C)(C)C)[nH]c2c1C(=O)NCC2]

To a stirred mixture of 3-[(3-fluoro-2-methoxyphenyl)amino]-2-(3-iodopyridin-4-yl)-1H,5H,6H,7H-pyrrolo[3,2-c]pyridin-4-one (150 mg, 0.31 mmol, 1.00 equiv) and Pd(dppf)Cl₂CH₂Cl₂ (63 mg, 0.08 mmol, 0.25 equiv) in DMF (2 mL) was added DIEA (202 mg, 1.58 mmol, 5.00 equiv) and tert-butyl (2R)-2-ethynyl-2-methylpyrrolidine-1-carboxylate (196 mg, 0.94 mmol, 3.00 equiv) at room temperature under argon atmosphere. The resulting mixture was stirred for 2 h at 50° C. under argon atmosphere. The residue was purified by reverse flash chromatography with the following conditions: mobile phase, MeCN in water, 10% to 70% gradient in 20 min; detector, UV 254 nm. This resulted in tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (150 mg, 85.46%) as a yellow solid.

LC-MS: (M+H)⁺ found: 560.1

### 108.2. Synthesis of 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one

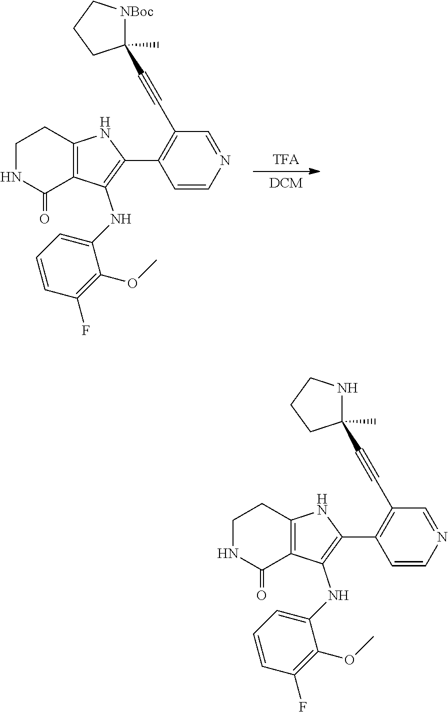 [SMILES: COc1c(F)cccc1Nc1c(-c2ccncc2C#C[C@@]2(C)CCCN2C(=O)OC(C)(C)C)[nH]c2c1C(=O)NCC2 ; COc1c(F)cccc1Nc1c(-c2ccncc2C#C[C@@]2(C)CCCN2)[nH]c2c1C(=O)NCC2]

To a stirred solution of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (120 mg, 0.21 mmol, 1.00 equiv) in DCM (3 mL) was added TFA (1 mL) dropwise at room temperature under nitrogen atmosphere. The resulting mixture was stirred for 1 h at room temperature under nitrogen atmosphere. The resulting oil was dried under nitrogen to afford 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (90 mg, 91.34%) as a yellow solid. The crude product was used in the next step directly without further purification.

LC-MS: (M+H)⁺ found: 460.05

### 108.3. Synthesis of 2-(3-{2-[(2R)-1-[(2E)-4-(dimethylamino)but-2-enoyl]-2-methylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one

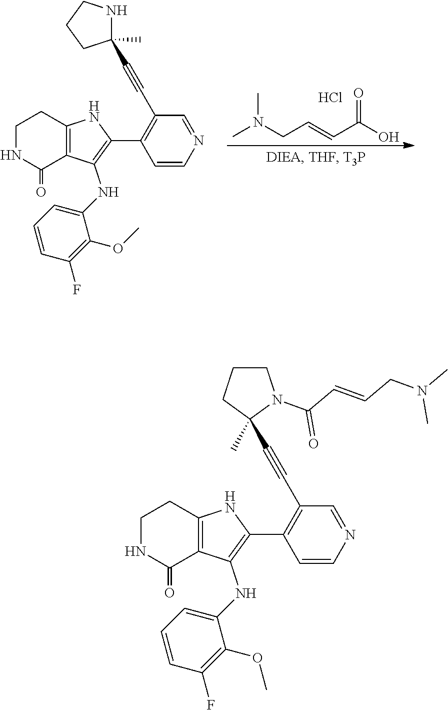

A solution of 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (60 mg, 0.13 mmol, 1.00 equiv) in THF (2 mL) was treated with DIEA (50 mg, 0.39 mmol, 3.00 equiv) for 10 min at 0° C. under nitrogen atmosphere followed by the addition of (2E)-4-(dimethylamino)but-2-enoic acid hydrochloride (33 mg, 0.26 mmol, 2.00 equiv) and T₃P (165 mg, 0.26 mmol, 2.00 equiv, 50% in EA) dropwise at 0° C. The resulting mixture was stirred for 1 h at room temperature under nitrogen atmosphere. The reaction was quenched by the addition of sat. NaHCO₃(aq.) (2 mL) at room temperature. The resulting mixture was extracted with EtOAc (3×10 mL). The combined organic layers were dried over anhydrous Na₂SO₄. After filtration, the filtrate was concentrated under reduced pressure. The crude product (100 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 48% B to 78% B in 7 min; Wave Length: 254 nm; RT1(min): 6.5; Number Of Runs: 0) to afford 2-(3-{2-[(2R)-1-[(2E)-4-(dimethylamino)but-2-enoyl]-2-methylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one (29.9 mg, 39.93%) as a yellow solid.

LC-MS: (M+H)⁺ found: 571.20

¹H NMR (400 MHz, Chloroform-d) δ 11.52 (s, 1H), 8.51 (s, 1H), 8.17 (d, 1H), 7.69 (s, 1H), 7.51-7.45 (m, 1H), 6.99-6.88 (m, 1H), 6.65-6.55 (m, 1H), 6.53-6.44 (m, 1H), 6.44-6.36 (m, 1H), 6.16-6.09 (m, 1H), 5.23 (s, 1H), 4.12 (d, 3H), 3.88-3.71 (m, 2H), 3.69-3.59 (m, 2H), 3.37-3.25 (m, 1H), 3.25-3.15 (m, 3H), 2.35 (s, 6H), 2.25-2.05 (m, 4H), 1.79 (s, 3H).

### Example 109. N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}but-2-ynamide; trifluoroacetic acid (compound 340)

### 109.1. Synthesis of 2-[3-(2-amino-2-methylpropoxy)pyridin-4-yl]-3-[(3-fluoro-2-methoxyphenyl)amino]-1H,5H,6H,7H-pyrrolo[3,2-c]pyridin-4-one

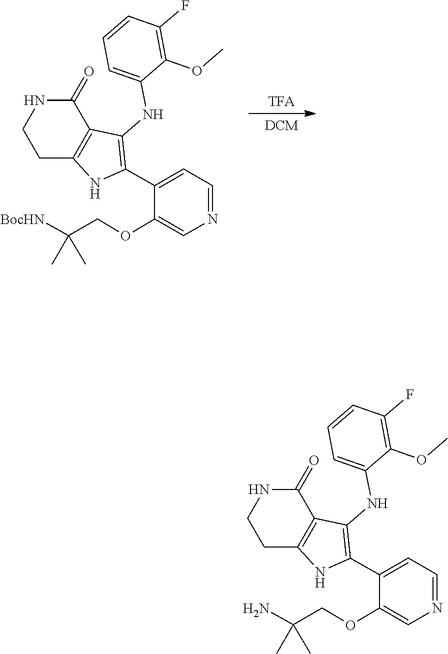 [SMILES: COc1c(F)cccc1Nc1c(-c2ccncc2OCC(C)(C)NC(=O)OC(C)(C)C)[nH]c2c1C(=O)NCC2 ; COc1c(F)cccc1Nc1c(-c2ccncc2OCC(C)(C)N)[nH]c2c1C(=O)NCC2]

To a stirred solution of tert-butyl N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}carbamate (710 mg, 1.32 mmol, 1.00 equiv) in DCM (6.00 mL) was added TFA (2.00 mL) dropwise at 0° C. The mixture was stirred for 1 h at room temperature under nitrogen atmosphere. The reaction was monitored by LCMS. The resulting mixture was concentrated under reduced pressure. The residue was purified by reverse flash chromatography with the following conditions: column, C18 spherical column; mobile phase, MeCN in water, 10% to 60% gradient in 15 min; detector, UV 254 nm to afford 2-[3-(2-amino-2-methylpropoxy)pyridin-4-yl]-3-[(3-fluoro-2-methoxyphenyl)amino]-1H,5H,6H,7H-pyrrolo[3,2-c]pyridin-4-one (460 mg, 79.55%) as a yellow solid. LC-MS: (M+H)⁺ found: 440.15.

### 109.2. Synthesis of N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}but-2-ynamide; trifluoroacetic acid

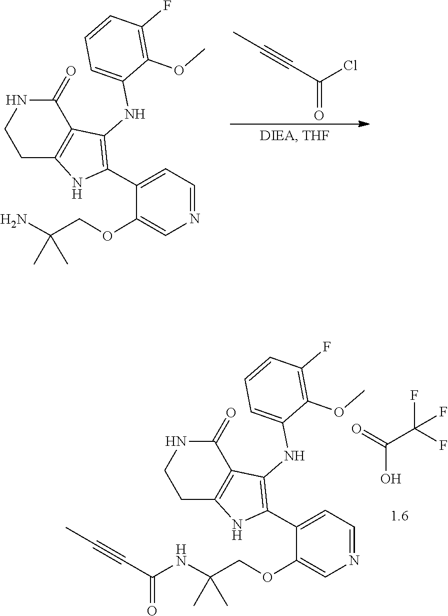

To a stirred solution of 2-[3-(2-amino-2-methylpropoxy)pyridin-4-yl]-3-[(3-fluoro-2-methoxyphenyl)amino]-1H,5H,6H,7H-pyrrolo[3,2-c]pyridin-4-one (60 mg, 0.14 mmol, 1.00 equiv) in THF (2.00 mL) was added DIEA (53 mg, 0.41 mmol, 3.00 equiv) and but-2-ynoyl chloride (14 mg, 0.14 mmol, 1.00 equiv) dropwise at 0° C. under nitrogen atmosphere. The reaction was monitored by LCMS. The resulting mixture was concentrated under reduced pressure. The resulting mixture was extracted with CH₂Cl₂:MeOH (10:1) (2×5 mL). The combined organic layers were dried over anhydrous Na₂SO₄. After filtration, the filtrate was concentrated under reduced pressure. The crude product (80 mg) was purified by Prep-HPLC with the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 11% B to 41% B in 7 min; Wave Length: 254 nm; RT1(min): 6.5; Number Of Runs: 0) to afford N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}but-2-ynamide; trifluoroacetic acid (36.2 mg, 42.41%) as a yellow solid.

LC-MS: (M+H)⁺ found: 506.2.

¹H NMR (300 MHz, Chloroform-d): δ 11.72 (s, 1H), 8.48 (brs, 1H), 8.01 (brs, 1H), 7.90 (d, 1H), 7.49 (d, 1H), 6.67-6.55 (m, 2H), 6.43 (brs, 1H), 6.23 (s, 1H), 5.95 (d, 1H), 4.22 (brs, 2H), 4.11 (s, 3H), 3.67 (t, 2H), 3.20 (t, 2H), 1.92 (s, 3H), 1.61 (s, 6H)

### Example 110. N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}prop-2-enamide (compound 339)

### 110.1. Synthesis of tert-butyl N-{1-[(4-bromopyridin-3-yl)oxy]-2-methylpropan-2-yl}carbamate

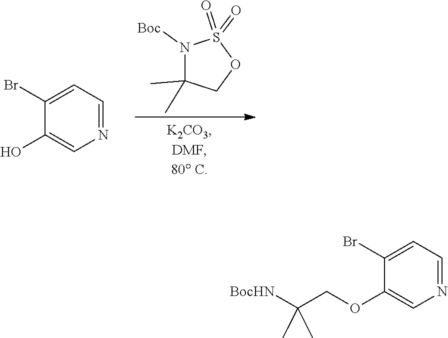 [SMILES: Oc1cnccc1Br ; CC(C)(C)OC(=O)N1C(C)(C)COS1(=O)=O ; CC(C)(COc1cnccc1Br)NC(=O)OC(C)(C)C]

To a stirred solution of 4-bromopyridin-3-ol (2.1 g, 12.14 mmol, 1.00 equiv) in DMF (20.00 mL) was added tert-butyl 4,4-dimethyl-2,2-dioxo-1,2lambda 6,3-oxathiazolidine-3-carboxylate (3.35 g, 13.35 mmol, 1.10 equiv) and K₂CO₃ (5.03 g, 36.42 mmol, 3.00 equiv) at room temperature. The mixture was stirred for 2h at 80° C. under nitrogen atmosphere. The reaction was monitored by LCMS. The mixture was allowed to cool down to room temperature. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (4:1) to afford tert-butyl N-{1-[(4-bromopyridin-3-yl)oxy]-2-methylpropan-2-yl}carbamate (4.0 g, 95.78%) as a light yellow solid.

LC-MS: (M+H)⁺ found: 344.85.

### 110.2. Synthesis of tert-butyl N-{2-methyl-1-[(4-{4-oxo-1H,5H,6H,7H-pyrrolo[3,2-c]pyridin-2-yl}pyridin-3-yl)oxy]propan-2-yl}carbamate

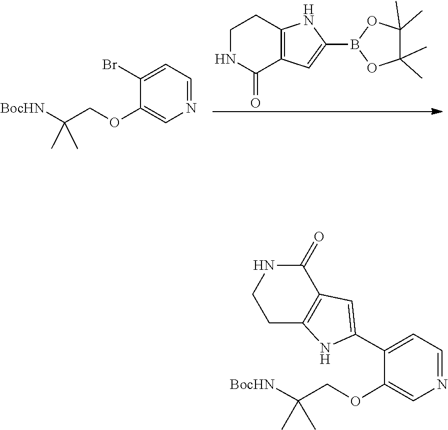 [SMILES: CC(C)(COc1cnccc1Br)NC(=O)OC(C)(C)C ; CC1(C)OB(c2cc3c([nH]2)CCNC3=O)OC1(C)C ; CC(C)(COc1cnccc1-c1cc2c([nH]1)CCNC2=O)NC(=O)OC(C)(C)C]

To a stirred solution of tert-butyl N-{1-[(4-bromopyridin-3-yl)oxy]-2-methylpropan-2-yl}carbamate (500 mg, 1.45 mmol, 1.00 equiv) in 1,4-dioxane (10 mL) was added 2-(4,4,5,5-tetramethyl-1,3,2-dioxaborolan-2-yl)-1H,5H,6H,7H-pyrrolo[3,2-c]pyridin-4-one (418 mg, 1.59 mmol, 1.10 equiv), Pd(dtbpf)C12 (95 mg, 0.15 mmol, 0.10 equiv) and Na₂CO₃ (461 mg, 4.34 mmol, 3.00 equiv) in 1,4-dioxane (10 mL) and H₂O (2.50 mL) at room temperature. The mixture was stirred for 2h at 50° C. under argon atmosphere. The reaction was monitored by LCMS. The resulting mixture was filtered, the filter cake was washed with 1,4-dioxane (3×8 mL). The filtrate was concentrated under reduced pressure. The residue was purified by reverse flash chromatography with the following conditions: column, C18 spherical column; mobile phase, MeCN in water, 10% to 50% gradient in 20 min; detector, UV 254 nm to afford tert-butyl N-{2-methyl-1-[(4-{4-oxo-1H,5H,6H,7H-pyrrolo[3,2-c]pyridin-2-yl}pyridin-3-yl)oxy]propan-2-yl}carbamate (490 mg, 84.48%) as a yellow solid.

LC-MS: (M+H)⁺ found: 401.2.

### 110.3. Synthesis of tert-butyl N-{1-[(4-{3-iodo-4-oxo-1H,5H,6H,7H-pyrrolo[3,2-c]pyridin-2-yl}pyridin-3-yl)oxy]-2-methylpropan-2-yl}carbamate

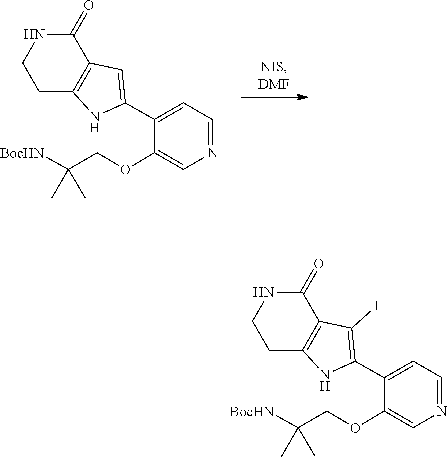 [SMILES: CC(C)(COc1cnccc1-c1cc2c([nH]1)CCNC2=O)NC(=O)OC(C)(C)C ; CC(C)(COc1cnccc1-c1[nH]c2c(c1I)C(=O)NCC2)NC(=O)OC(C)(C)C]

To a stirred solution of tert-butyl N-{2-methyl-1-[(4-{4-oxo-1H,5H,6H,7H-pyrrolo[3,2-c]pyridin-2-yl}pyridin-3-yl)oxy]propan-2-yl}carbamate (440 mg, 1.10 mmol, 1.00 equiv) in DMF (5.00 mL) was added NIS (247 mg, 1.10 mmol, 1.00 equiv) dropwise at 0° C. The mixture was stirred for 1 h at room temperature under nitrogen atmosphere. The reaction was monitored by LCMS. The reaction was quenched with sat. Na₂SO₃ (aq.) at 0° C. The residue was purified by reverse flash chromatography with the following conditions: column, C18 spherical column; mobile phase, MeCN in water, 10% to 70% gradient in 20 min; detector, UV 254 nm to afford tert-butyl N-{1-[(4-{3-iodo-4-oxo-1H,5H,6H,7H-pyrrolo[3,2-c]pyridin-2-yl}pyridin-3-yl)oxy]-2-methylpropan-2-yl}carbamate (380 mg, 65.71%) as a yellow green solid.

LC-MS: (M+H)⁺ found: 527.15.

### 110.4. Synthesis of tert-butyl N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}carbamate

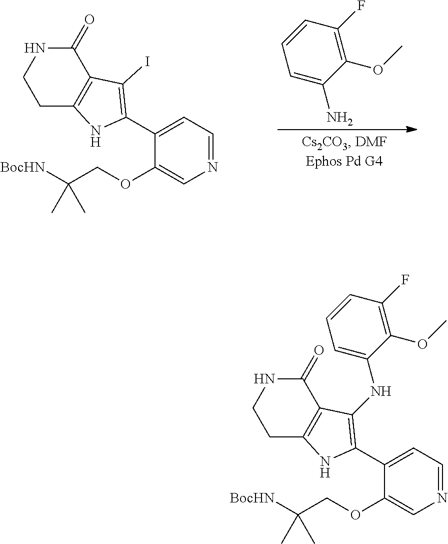

To a stirred solution of tert-butyl N-{1-[(4-{3-iodo-4-oxo-1H,5H,6H,7H-pyrrolo[3,2-c]pyridin-2-yl}pyridin-3-yl)oxy]-2-methylpropan-2-yl}carbamate (350 mg, 0.66 mmol, 1.00 equiv) in DMF (3.00 mL) was added EPhos Pd G4 (57 mg, 0.07 mmol, 0.10 equiv), Cs₂CO₃ (650 mg, 1.99 mmol, 3.00 equiv) and 3-fluoro-2-methoxyaniline (280 mg, 1.99 mmol, 3.00 equiv) at room temperature. The mixture was stirred for 2h at 50° C. under argon atmosphere. The reaction was monitored by LCMS. The resulting mixture was concentrated under reduced pressure. The residue was purified by silica gel column chromatography, eluted with CH₂Cl₂/MeOH (93:7) to afford tert-butyl N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}carbamate (330 mg, 91.79%) as a yellow solid.

LC-MS: (M+H)⁺ found: 540.35.

### 110.5. Synthesis of 2-[3-(2-amino-2-methylpropoxy)pyridin-4-yl]-3-[(3-fluoro-2-methoxyphenyl)amino]-1H,5H,6H,7H-pyrrolo[3,2-c]pyridin-4-one

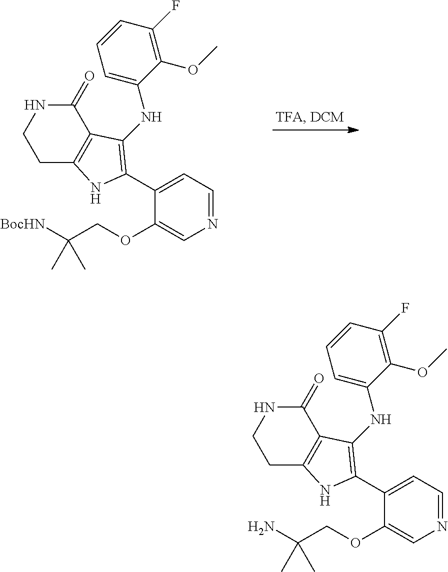 [SMILES: COc1c(F)cccc1Nc1c(-c2ccncc2OCC(C)(C)NC(=O)OC(C)(C)C)[nH]c2c1C(=O)NCC2 ; COc1c(F)cccc1Nc1c(-c2ccncc2OCC(C)(C)N)[nH]c2c1C(=O)NCC2]

To a stirred solution of tert-butyl N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}carbamate (290 mg, 0.54 mmol, 1.00 equiv) in DCM (4.00 mL) was added TFA (1.00 mL) dropwise at 0° C. The mixture was stirred for 1 h at room temperature under nitrogen atmosphere. The reaction was monitored by LCMS. The resulting mixture was concentrated under nitrogen atmosphere. The residue was purified by reverse flash chromatography with the following conditions: C18 spherical column; mobile phase, MeCN in water, 10% to 60% gradient in 15 min; detector, UV 254 nm to afford 2-[3-(2-amino-2-methylpropoxy)pyridin-4-yl]-3-[(3-fluoro-2-methoxyphenyl)amino]-1H,5H,6H,7H-pyrrolo[3,2-c]pyridin-4-one (140 mg, 59.27%) as a yellow solid.

LC-MS: (M+H)⁺ found: 440.10.

### 110.6. Synthesis of N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}prop-2-enamide

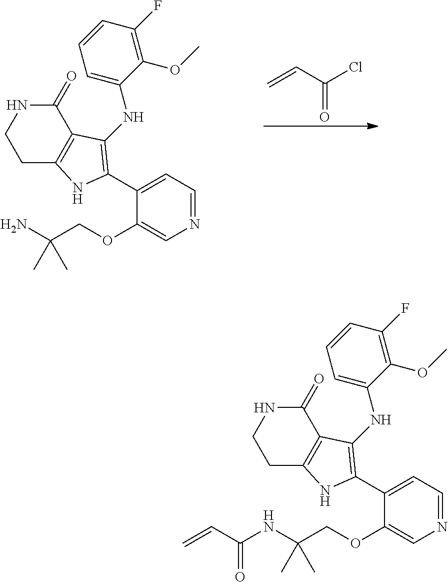

To a stirred solution of 2-[3-(2-amino-2-methylpropoxy)pyridin-4-yl]-3-[(3-fluoro-2-methoxyphenyl)amino]-1H,5H,6H,7H-pyrrolo[3,2-c]pyridin-4-one (120 mg, 0.27 mmol, 1.00 equiv) in THF (3.00 mL) was added DIEA (106 mg, 0.82 mmol, 3.00 equiv) at 0° C. Acryloyl chloride (24 mg, 0.27 mmol, 1.00 equiv) was added dropwise at 0° C. The mixture was stirred for 1 h at room temperature under nitrogen atmosphere. The reaction was monitored by LCMS. The resulting mixture was concentrated under reduced pressure. The residue was purified by silica gel column chromatography, eluted with CH₂Cl₂/MeOH (93:7) to afford crude product. The crude product (80 mg) was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 18% B to 43% B in 9 min, 43% B; Wave Length: 254/220 nm; RT1(min): 8.85; Number Of Runs: 0) to afford N-{1-[(4-{3-[(3-fluoro-2-methoxyphenyl)amino]-4-oxo-1H,5H,6H,7H-pyrrolo[3,2-c]pyridin-2-yl}pyridin-3-yl)oxy]-2-methylpropan-2-yl}prop-2-enamide (14.4 mg, 10.59%) as a light yellow solid.

LC-MS: (M+H)⁺ found: 494.10.

¹H NMR (300 MHz, DMSO-d6): δ 11.22 (s, 1H), 8.39 (s, 1H), 8.02-7.99 (m, 2H), 7.42 (s, 1H), 7.30 (d, J=4.8 Hz, 1H), 7.12 (s, 1H), 6.66-6.59 (m, 1H), 6.51-6.44 (m, 1H), 6.37-6.28 (m, 1H), 6.11-6.00 (m, 2H), 5.58-5.54 (m, 1H), 4.30 (s, 2H), 3.90 (s, 3H), 3.43-3.40 (m, 2H), 2.87 (t, J=6.6 Hz, 2H), 1.42 (s, 6H).

### Example 111. 2-(3-{2-[(6R)-5-[(2E)-4-(dimethylamino)but-2-enoyl]-5-azaspiro[2.4]heptan-6-yl]ethynyl}pyridin-4-yl)-3-[(3-fluoro-2-methoxyphenyl)amino]-1H,5H,6H,7H-pyrrolo[3,2-c]pyridin-4-one (compound 343)

### 111.1. Synthesis of tert-butyl (6R)-6-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-azaspiro[2.4]heptane-5-carboxylate

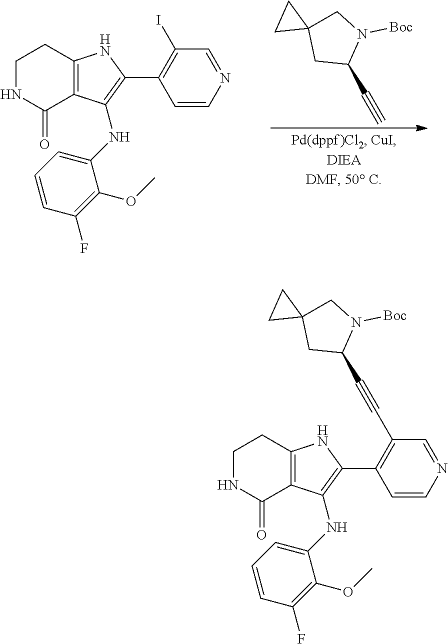 [SMILES: COc1c(F)cccc1Nc1c(-c2ccncc2I)[nH]c2c1C(=O)NCC2 ; C#C[C@H]1CC2(CC2)CN1C(=O)OC(C)(C)C ; COc1c(F)cccc1Nc1c(-c2ccncc2C#C[C@H]2CC3(CC3)CN2C(=O)OC(C)(C)C)[nH]c2c1C(=O)NCC2]

To a stirred solution of 3-[(3-fluoro-2-methoxyphenyl)amino]-2-(3-iodopyridin-4-yl)-1H,5H,6H,7H-pyrrolo[3,2-c]pyridin-4-one (130 mg, 0.27 mmol, 1.00 equiv) and Pd(dppf)Cl₂CH₂Cl₂ (55 mg, 0.07 mmol, 0.25 equiv), CuI (25 mg, 0.13 mmol, 0.50 equiv) in DMF (1.5 mL) were added tert-butyl (6R)-6-ethynyl-5-azaspiro[2.4]heptane-5-carboxylate (300 mg, 1.36 mmol, 5.00 equiv) and DIEA (175 mg, 1.36 mmol, 5.00 equiv) at room temperature under Ar atmosphere. The resulting mixture was stirred for 2 h at 50° C. under Ar atmosphere in a sealed tube. The residue was purified by reverse phase flash to afford tert-butyl (6R)-6-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-azaspiro[2.4]heptane-5-carboxylate (80 mg, 51.49%) as a yellow solid.

LC-MS: (M+H)⁺ found: 572.20.

### 111.2. Synthesis of (R)-2-(3-((5-azaspiro[2.4]heptan-6-yl)ethynyl)pyridin-4-yl)-3-((3-fluoro-2-methoxyphenyl)amino)-1,5,6,7-tetrahydro-4H-pyrrolo[3,2-c]pyridin-4-one

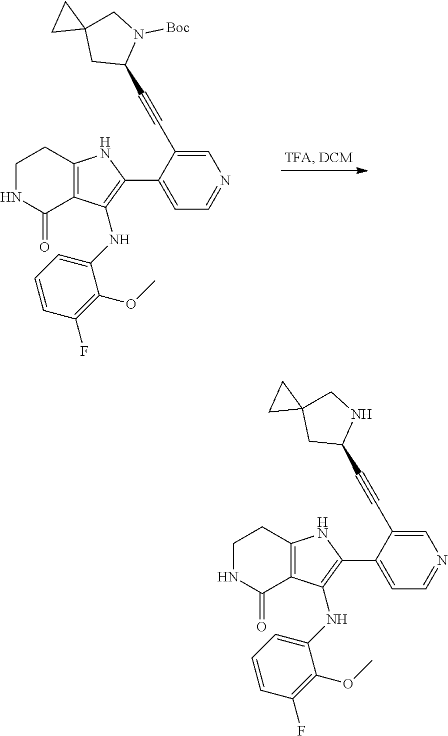 [SMILES: COc1c(F)cccc1Nc1c(-c2ccncc2C#C[C@H]2CC3(CC3)CN2C(=O)OC(C)(C)C)[nH]c2c1C(=O)NCC2 ; COc1c(F)cccc1Nc1c(-c2ccncc2C#C[C@H]2CC3(CC3)CN2)[nH]c2c1C(=O)NCC2]

To a stirred solution of tert-butyl (6R)-6-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-azaspiro[2.4]heptane-5-carboxylate (80 mg, 0.14 mmol, 1.00 equiv) in DCM (2.00 mL) was added TFA (0.80 mL) dropwise at 0 C. The resulting mixture was stirred for 2 h at room temperature. The mixture was allowed to dry using nitrogen. The crude product was used in the next step directly without further purification.

LC-MS: (M+H)⁺ found: 472.20.

### 111.3. Synthesis of 2-(3-{2-[(6R)-5-[(2E)-4-(dimethylamino)but-2-enoyl]-5-azaspiro[2.4]heptan-6-yl]ethynyl}pyridin-4-yl)-3-[(3-fluoro-2-methoxyphenyl)amino]-1H,5H,6H,7H-pyrrolo[3,2-c]pyridin-4-one

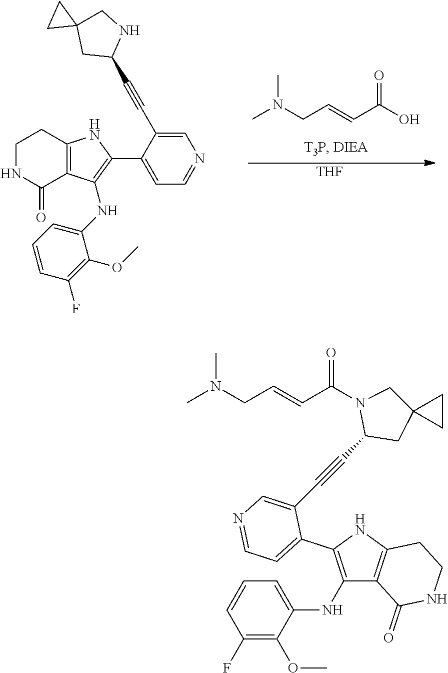

To a stirred solution of 2-(3-{2-[(6R)-5-azaspiro[2.4]heptan-6-yl]ethynyl}pyridin-4-yl)-3-[(3-fluoro-2-methoxyphenyl)amino]-1H,5H,6H,7H-pyrrolo[3,2-c]pyridin-4-one (60 mg, 0.12 mmol, 1.00 equiv) and (2E)-4-(dimethylamino)but-2-enoic acid hydrochloride (42 mg, 0.25 mmol, 2.00 equiv) in THF were added T₃P (202 mg, 0.31 mmol, 2.50 equiv, 50% in EA) and DIEA (0.8 mL) dropwise at 0° C. The resulting mixture was extracted with EtOAc (3×5 mL). The combined organic layers were washed with NaHCO₃(aq.) (1×10 mL), dried over anhydrous Na₂SO₄. After filtration, the filtrate was concentrated under reduced pressure. The crude product was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 11% B to 27% B in 8 min, 27% B; Wave Length: 254/220 nm; RT1(min): 8; Number Of Runs: 0) to afford 2-(3-{2-[(6R)-5-[(2E)-4-(dimethylamino)but-2-enoyl]-5-azaspiro[2.4]heptan-6-yl]ethynyl}pyridin-4-yl)-3-[(3-fluoro-2-methoxyphenyl)amino]-1H,5H,6H,7H-pyrrolo[3,2-c]pyridin-4-one (30 mg) as a yellow solid. The product (30 mg) was then separated by Prep-Chiral-HPLC with the following conditions (Column: CHIRAL ART Cellulose-SC, 2*25 cm, 5 μm; Mobile Phase A: Hex:DCM=3:1(0.5% 2M NH₃-MeOH)—HPLC, Mobile Phase B: EtOH—HPLC; Flow rate: 20 mL/min; Gradient: 30% B to 30% B in 11 min; Wave Length: 220/254 nm; RT1(min): 8.59; RT2(min): 9.39; Sample Solvent: ETOH:DCM=1:1; Injection Volume: 0.5 mL; Number Of Runs: 7) to afford 2-(3-{2-[(6R)-5-[(2E)-4-(dimethylamino)but-2-enoyl]-5-azaspiro[2.4]heptan-6-yl]ethynyl}pyridin-4-yl)-3-[(3-fluoro-2-methoxyphenyl)amino]-1H,5H,6H,7H-pyrrolo[3,2-c]pyridin-4-one (23.1 mg, 33.07%) as a yellow solid.

LC-MS: (M+H)⁺ found: 583.3.

¹H NMR (400 MHz, Chloroform-d) δ 11.09 (s, 1H), 8.53 (s, 1H), 8.16 (d, 1H), 7.69 (s, 1H), 7.47 (d, 1H), 6.99-6.95 (m, 1H), 6.58-6.43 (m, 3H), 6.09 (d, 1H), 5.20 (s, 1H), 5.03-5.00 (m, 1H), 4.10 (s, 3H), 3.70-3.61 (m, 4H), 3.39-3.37 (m, 2H), 3.27 (t, 2H), 2.66 (s, 1H), 2.52 (s, 5H), 2.33-2.27 (m, 1H), 2.18-2.14 (m, 1H), 0.90-0.81 (m, 2H), 0.71 (t, 2H).

### Example 112. 3-[(3-chloro-2-methoxyphenyl)amino]-2-(3-{[(2S)-1-(2-fluoroprop-2-enoyl)azetidin-2-yl]methoxy}pyridin-4-yl)-1H,5H,6H,7H-pyrrolo[3,2-c]pyridin-4-one (compound 363)

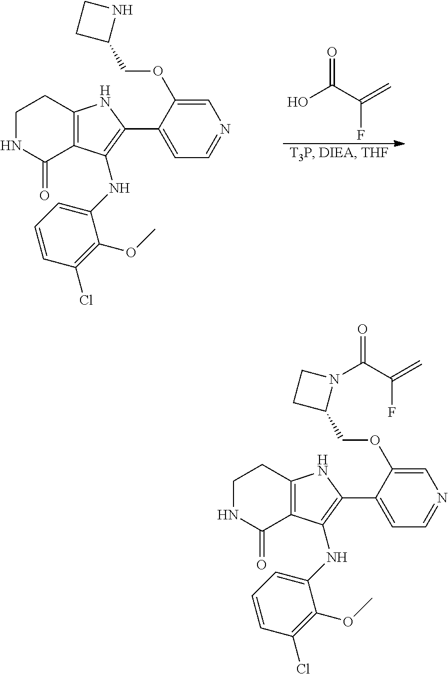

Into a 50 mL round-bottom flask were added 2-{3-[(2S)-azetidin-2-ylmethoxy]pyridin-4-yl}-3-[(3-chloro-2-methoxyphenyl)amino]-1H,5H,6H,7H-pyrrolo[3,2-c]pyridin-4-one (60 mg, 0.13 mmol, 1.00 equiv) and THF (2 mL) at room temperature. To the above mixture was basified to pH 8 with DIEA (52 mg, 0.39 mmol, 3.00 equiv) at 0° C. Then, to the above mixture was added 2-fluoroprop-2-enoic acid (18 mg, 0.20 mmol, 1.50 equiv) and T₃P (169 mg, 0.26 mmol, 2.00 equiv, 50% in EA) at 0° C. The resulting mixture was stirred for additional 1 h at room temperature. The reaction was monitored by LCMS. The resulting mixture was extracted with DCM:MeOH=10:1 (4×20 mL). The combined organic layers were washed with NaHCO₃(aq.) (1×20 mL) and dried over anhydrous Na₂SO₄. After filtration, the filtrate was concentrated under reduced pressure. The crude product (178 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 26% B to 51% B in 9 min, 51% B; Wave Length: 254/220 nm; RT1(min): 8.85; Number Of Runs: 0) to afford 3-[(3-chloro-2-methoxyphenyl)amino]-2-(3-{[(2S)-1-(2-fluoroprop-2-enoyl)azetidin-2-yl]methoxy}pyridin-4-yl)-1H,5H,6H,7H-pyrrolo[3,2-c]pyridin-4-one (10.2 mg, 14.67%) as a light yellow solid.

LC-MS: M+H found: 526.05.

¹H NMR (400 MHz, Chloroform-d) δ 11.67 (s, 1H), 8.28 (s, 1H), 7.95 (d, 1H), 7.89 (s, 1H), 7.46 (d, 1H), 6.79-6.75 (m, 1H), 6.66 (t, 1H), 6.19-6.15 (m, 1H), 5.68-5.56 (m, 1H), 5.30-5.24 (m, 2H), 5.15-5.05 (m, 1H), 4.65-4.42 (m, 3H), 4.33 (d, 1H), 4.11 (s, 3H), 3.68-3.60 (m, 2H), 3.20-3.12 (m, 2H), 2.80-2.66 (m, 1H), 2.15-2.25 (m, 1H)

### Example 113. 3-[(3-chloro-2-methoxyphenyl)amino]-2-(3-{2-[(2R)-1-[(2E)-4-(dimethylamino)but-2-enoyl]-2-methylpyrrolidin-2-yl]ethynyl}pyridin-4-yl)-1H,5H,6H,7H-pyrrolo[3,2-c]pyridin-4-one (compound 362)

### 113.1. Synthesis of tert-butyl (2R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate

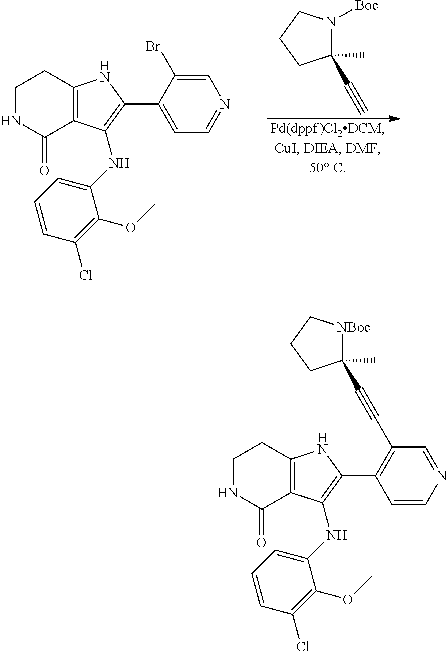 [SMILES: COc1c(Cl)cccc1Nc1c(-c2ccncc2Br)[nH]c2c1C(=O)NCC2 ; C#C[C@@]1(C)CCCN1C(=O)OC(C)(C)C ; "Pd(dppf)Cl2·DCM,CuI, DIEA, DMF,50° C." ; COc1c(Cl)cccc1Nc1c(-c2ccncc2C#C[C@@]2(C)CCCN2C(=O)OC(C)(C)C)[nH]c2c1C(=O)NCC2]

To a stirred mixture of 2-(3-bromopyridin-4-yl)-3-[(3-chloro-2-methoxyphenyl)amino]-1H,5H,6H,7H-pyrrolo[3,2-c]pyridin-4-one (300 mg, 0.67 mmol, 1.00 equiv) and CuI (63 mg, 0.33 mmol, 0.50 equiv) and Pd(dppf)Cl₂·CH₂Cl₂ (136 mg, 0.17 mmol, 0.25 equiv) in DMF (5 mL) were added tert-butyl (2R)-2-ethynyl-2-methylpyrrolidine-1-carboxylate (28 mg, 0.14 mmol, 3.00 equiv) and DIEA (259 mg, 2.01 mmol, 3.00 equiv) dropwise at room temperature under argon atmosphere. The resulting mixture was stirred for overnight at 50° C. under argon atmosphere. The residue was purified by reverse flash chromatography with the following conditions: column, C18 spherical column 1; mobile phase, MeCN in water, 10% to 70% gradient in 30 min; detector, UV 254 nm to afford tert-butyl (2R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (290 mg, 75.13%) as a yellow solid.

LC-MS: (M+H)⁺ found: 576.15.

### 113.2. Synthesis of (R)-3-((3-chloro-2-methoxyphenyl)amino)-2-(3-((2-methylpyrrolidin-2-yl)ethynyl)pyridin-4-yl)-1,5,6,7-tetrahydro-4H-pyrrolo[3,2-c]pyridin-4-one

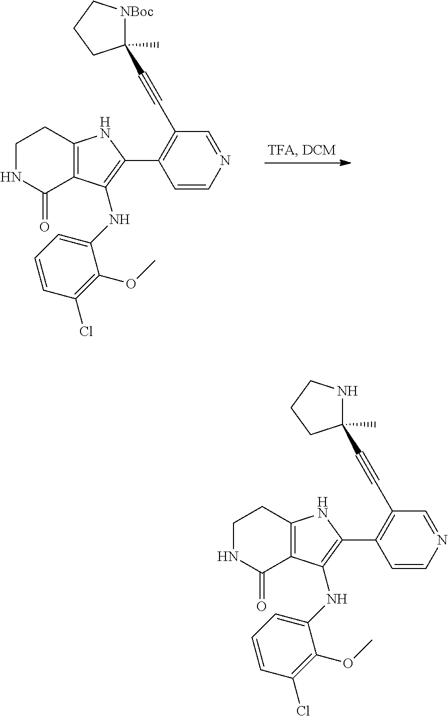 [SMILES: COc1c(Cl)cccc1Nc1c(-c2ccncc2C#C[C@@]2(C)CCCN2C(=O)OC(C)(C)C)[nH]c2c1C(=O)NCC2 ; COc1c(Cl)cccc1Nc1c(-c2ccncc2C#C[C@@]2(C)CCCN2)[nH]c2c1C(=O)NCC2]

To a stirred solution of tert-butyl (2R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (80 mg, 0.13 mmol, 1. 00 equiv) in DCM (2 mL) was added TFA (0.5 mL) dropwise at room temperature under nitrogen atmosphere. The resulting mixture was stirred for 1 h at room temperature under nitrogen atmosphere. The resulting oil was dried under nitrogen. The crude product was used in the next step directly without further purification.

LC-MS: (M+H)⁺ found: 476.10

### 113.3. Synthesis of 3-[(3-chloro-2-methoxyphenyl)amino]-2-(3-{2-[(2R)-1-[(2E)-4-(dimethylamino)but-2-enoyl]-2-methylpyrrolidin-2-yl]ethynyl}pyridin-4-yl)-1H,5H,6H,7H-pyrrolo[3,2-c]pyridin-4-one

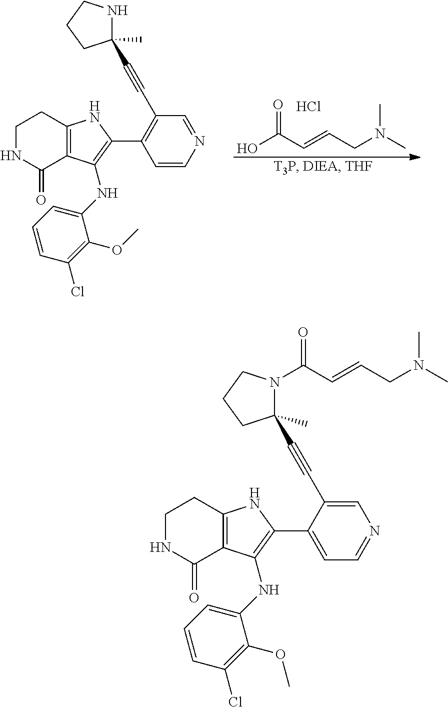

To a stirred solution of 3-[(3-chloro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (82 mg, 0.17 mmol, 1.00 equiv) and (2E)-4-(dimethylamino)but-2-enoic acid hydrochloride (56 mg, 0.34 mmol, 2.00 equiv) in THF (4 mL) was added DIEA (44 mg, 0.34 mmol, 2.00 equiv) and T₃P (216 mg, 0.34 mmol, 2.00 equiv, 50% in EA) dropwise at 0° C. under nitrogen atmosphere. The resulting mixture was stirred for 4 h at room temperature under nitrogen atmosphere. The resulting mixture was washed with 3×10 mL of NaHCO₃(aq). The resulting mixture was extracted with EtOAc (3×20 mL). The combined organic layers were dried over anhydrous Na₂SO₄. After filtration, the filtrate was concentrated under reduced pressure. The crude product (100 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 8% B to 30% B in 10 min, 30% B; Wave Length: 254/220 nm; RT1(min): 9; Number Of Runs: 0) to afford 3-[(3-chloro-2-methoxyphenyl)amino]-2-(3-{2-[(2R)-1-[(2E)-4-(dimethylamino)but-2-enoyl]-2-methylpyrrolidin-2-yl]ethynyl}pyridin-4-yl)-1H,5H,6H,7H-pyrrolo[3,2-c]pyridin-4-one (27.1 mg, 26.50%) as a yellow solid.

LC-MS: (M+H)⁺ found: 587.2

¹H NMR (300 MHz, Chloroform-d) δ 11.49 (s, 1H), 8.52 (s, 1H), 8.14 (d, J=5.6 Hz, 1H), 7.66 (s, 1H), 7.40 (d, J=5.6 Hz, 1H), 7.04-6.83 (m, 1H), 6.78-6.66 (m, 1H), 6.61 (t, J=8.1 Hz, 1H), 6.46-6.41 (m, 1H), 6.26-6.18 (m, 1H), 5.34 (s, 1H), 4.07 (s, 3H), 3.88-3.70 (m, 2H), 3.63-3.59 (m, 2H), 3.35-3.10 (m, 4H), 2.41 (s, 6H), 2.24-2.03 (m, 4H), 1.76 (s, 3H).

### Example 114. 3-[(3-chloro-5-fluoro-2-methoxyphenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one (compound 366)

### 114.1. Synthesis of tert-butyl (2R)-2-{[(4-{3-[(3-chloro-5-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate

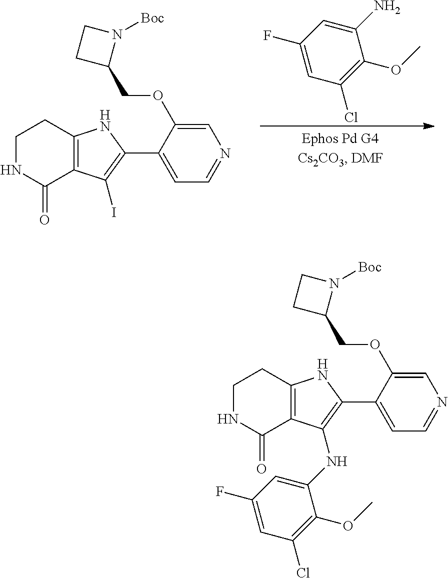 [SMILES: CC(C)(C)OC(=O)N1CC[C@@H]1COc1cnccc1-c1[nH]c2c(c1I)C(=O)NCC2 ; COc1c(N)cc(F)cc1Cl ; COc1c(Cl)cc(F)cc1Nc1c(-c2ccncc2OC[C@H]2CCN2C(=O)OC(C)(C)C)[nH]c2c1C(=O)NCC2]

To a stirred solution of tert-butyl (2R)-2-{[(4-{3-iodo-4-oxo-1H,5H,6H,7H-pyrrolo[3,2-c]pyridin-2-yl}pyridin-3-yl)oxy]methyl}azetidine-1-carboxylate (320 mg, 0.61 mmol, 1.00 equiv) and 3-chloro-5-fluoro-2-methoxyaniline (118 mg, 0.67 mmol, 1.10 equiv) in DMF (4 mL) were added Ephos Pd G4 (56 mg, 0.06 mmol, 0.10 equiv) and Cs₂CO₃ (597 mg, 1.83 mmol, 3.00 equiv) at room temperature. The resulting mixture was stirred for 2h at 50° C. under argon atmosphere. The reaction was monitored by LCMS. The resulting mixture was extracted with DCM:MeOH (10:1, 4×20 mL). The combined organic layers were dried over anhydrous Na₂SO₄. After filtration, the filtrate was concentrated under reduced pressure. The residue was purified by silica gel column chromatography, eluted with DCM:MeOH (10:1) to afford tert-butyl (2R)-2-{[(4-{3-[(3-chloro-5-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (222 mg, 63.74%) as a yellow solid.

LC-MS: M+H found: 572.15.

### 114.2. Synthesis of 2-{3-[(2R)-azetidin-2-ylmethoxy]pyridin-4-yl}-3-[(3-chloro-5-fluoro-2-methoxyphenyl)amino]-1H,5H,6H,7H-pyrrolo[3,2-c]pyridin-4-one

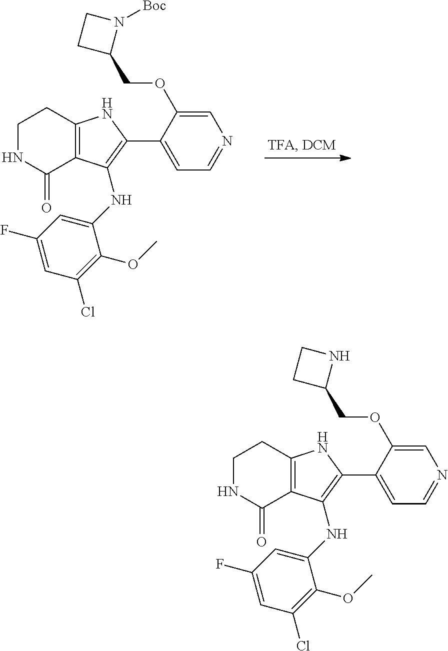 [SMILES: COc1c(Cl)cc(F)cc1Nc1c(-c2ccncc2OC[C@H]2CCN2C(=O)OC(C)(C)C)[nH]c2c1C(=O)NCC2 ; COc1c(Cl)cc(F)cc1Nc1c(-c2ccncc2OC[C@H]2CCN2)[nH]c2c1C(=O)NCC2]

Into a 25 mL round-bottom flask were added tert-butyl (2R)-2-{[(4-{3-[(3-chloro-5-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (260 mg, 0.45 mmol, 1.00 equiv) and DCM (3 mL) at room temperature. To the above mixture was added TFA (2 mL) dropwise over 0.5 min at 0° C. The resulting mixture was stirred for additional 1 h at room temperature. The reaction was monitored by LCMS. The resulting mixture was concentrated under reduced pressure. The residue was purified by reverse flash chromatography with the following conditions (column, C18 spherical column; mobile phase, MeCN in water, 10% to 50% gradient in 30 min; detector, UV 254 nm) to afford 2-{3-[(2R)-azetidin-2-ylmethoxy]pyridin-4-yl}-3-[(3-chloro-5-fluoro-2-methoxyphenyl)amino]-1H,5H,6H,7H-pyrrolo[3,2-c]pyridin-4-one (146 mg, 68.20%) as a brown solid.

LC-MS: (M+H)⁺ found 472.00.

### 114.3. Synthesis of 3-[(3-chloro-5-fluoro-2-methoxyphenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one

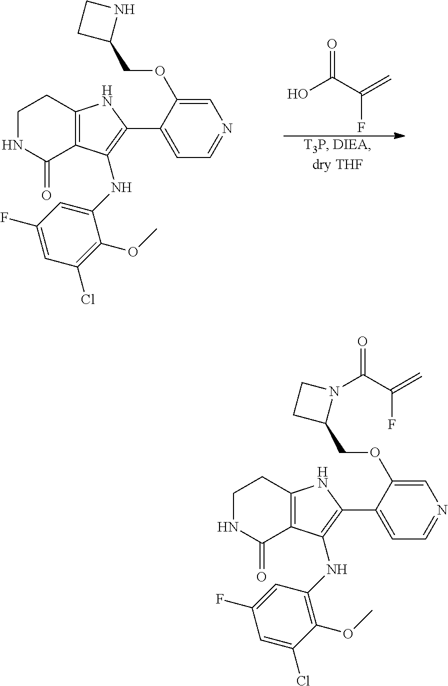

Into a 8 mL vial were added 2-{3-[(2R)-azetidin-2-ylmethoxy]pyridin-4-yl}-3-[(3-chloro-5-fluoro-2-methoxyphenyl)amino]-1H,5H,6H,7H-pyrrolo[3,2-c]pyridin-4-one (75 mg, 0.16 mmol, 1.00 equiv) and THF (1.5 mL) at room temperature. The mixture was basified to pH 9 with DIEA (123 mg, 0.95 mmol, 6.00 equiv) at 0° C. To the above mixture was added 2-fluoroprop-2-enoic acid (43 mg, 0.48 mmol, 3.00 equiv) and T₃P (405 mg, 0.64 mmol, 4.00 equiv, 50% in EA) at 0° C. The resulting mixture was stirred for additional 1 h at room temperature. The reaction was monitored by LCMS. The resulting mixture was extracted with CH₂Cl₂:MeOH (10:1, 4×20 mL). The combined organic layers were washed with NaHCO₃(aq.) (1×20 mL) and dried over anhydrous Na₂SO₄. After filtration, the filtrate was concentrated under reduced pressure. The crude product (112 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 29% B to 48% B in 10 min, 48% B; Wave Length: 254/220 nm; RT1(min): 9.67; Number Of Runs: 0) to afford 3-[(3-chloro-5-fluoro-2-methoxyphenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one (18.0 mg, 20.54%) as oyster white solid.

LC-MS: (M+H)⁺ found 544.1

¹H NMR (400 MHz, Chloroform-d) δ 11.61 (s, 1H), 8.30 (s, 1H), 8.03 (d, 1H), 7.56-7.46 (m, 2H), 6.46-6.43 (m, 1H), 5.97-5.94 (m, 1H), 5.68-5.56 (m, 1H), 5.26-5.16 (m, 3H), 4.64-4.39 (m, 3H), 4.32-4.29 (m, 1H), 4.05 (s, 3H), 3.63-3.59 (m, 2H), 3.13-3.10 (m, 2H), 2.77-2.63 (m, 1H), 2.22-2.17 (m, 1H).

### Example 115. N-[(2R)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]prop-2-enamide (compound 377)

### 115.1. Synthesis of tert-butyl N-[(2R)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]carbamate

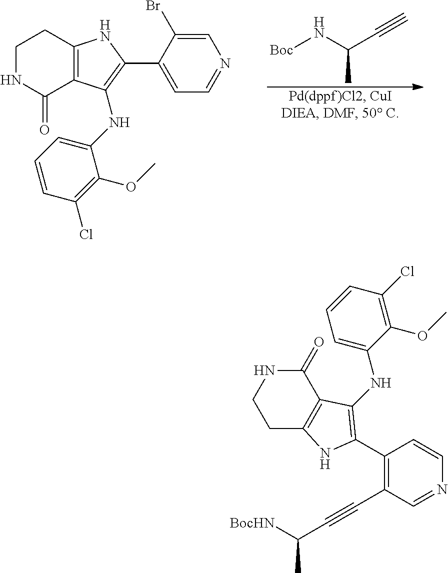

To a stirred solution of 2-(3-bromopyridin-4-yl)-3-[(3-chloro-2-methoxyphenyl)amino]-1H,5H,6H,7H-pyrrolo[3,2-c]pyridin-4-one (250 mg, 0.56 mmol, 1.00 equiv) and tert-butyl N-[(2R)-but-3-yn-2-yl]carbamate (236 mg, 1.40 mmol, 2.50 equiv) in DMF (3.00 mL) was added DIEA (180 mg, 1.40 mmol, 2.50 equiv), CuI (53 mg, 0.28 mmol, 0.50 equiv) and Pd(dppf)Cl₂CH₂Cl₂ (114 mg, 0.14 mmol, 0.25 equiv) dropwise at 0° C. under Ar atmosphere. The resulting mixture was stirred for 1 h at 50° C. under Ar atmosphere. The reaction was monitored by LCMS. The residue was purified by reverse flash chromatography with the following conditions: column, C18 spherical column; mobile phase, ACN in water, 0% to 100% gradient in 30 min; detector, UV 254 nm, then the residue was purified by silica gel column chromatography, eluted with MeOH/DCM (3%) to afford tert-butyl N-[(2R)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]carbamate (190 mg, 60.31%) as a yellow solid.

LC-MS: (M+H)⁺ found: 536.20.

### 115.2. Synthesis of 2-{3-[(3R)-3-aminobut-1-yn-1-yl]pyridin-4-yl}-3-[(3-chloro-2-methoxyphenyl)amino]-1H,5H,6H,7H-pyrrolo[3,2-c]pyridin-4-one

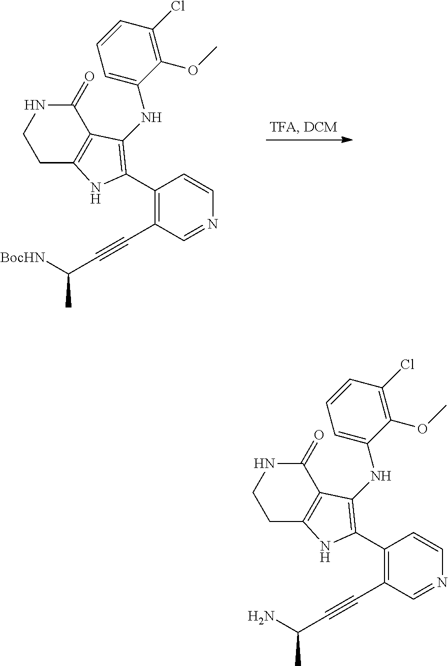 [SMILES: COc1c(Cl)cccc1Nc1c(-c2ccncc2C#C[C@@H](C)NC(=O)OC(C)(C)C)[nH]c2c1C(=O)NCC2 ; COc1c(Cl)cccc1Nc1c(-c2ccncc2C#C[C@@H](C)N)[nH]c2c1C(=O)NCC2]

Into a DCM (3.00 mL) were added tert-butyl N-[(2R)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]carbamate (250 mg, 0.47 mmol, 1.00 equiv) and TFA (1.00 mL) at 0° C. The resulting mixture was stirred for 1 h at 25° C. The reaction was monitored by LCMS. The resulting mixture was concentrated under reduced pressure. The residue was purified by reverse flash chromatography with the following conditions: column, C18 spherical column; mobile phase, ACN in water, 0% to 100% gradient in 30 min; detector, UV 254 nm to afford 2-{3-[(3R)-3-aminobut-1-yn-1-yl]pyridin-4-yl}-3-[(3-chloro-2-methoxyphenyl)amino]-1H,5H,6H,7H-pyrrolo[3,2-c]pyridin-4-one (150 mg, 70.09%) as a yellow solid.

LC-MS: (M+H)⁺ found: 436.00.

### 115.3. Synthesis of N-[(2R)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]prop-2-enamide

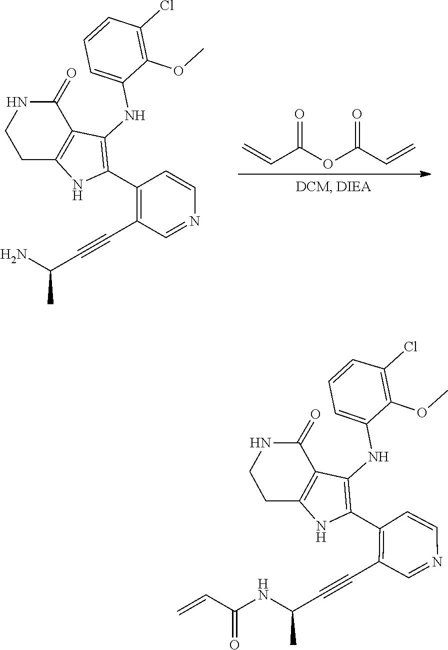

A solution of 2-{3-[(3R)-3-aminobut-1-yn-1-yl]pyridin-4-yl}-3-[(3-chloro-2-methoxyphenyl)amino]-1H,5H,6H,7H-pyrrolo[3,2-c]pyridin-4-one (100 mg, 0.23 mmol, 1.00 equiv) and DIEA (148 mg, 1.15 mmol, 5.00 equiv) in DCM (1.50 mL) was added prop-2-enoyl prop-2-enoate (58 mg, 0.46 mmol, 2.00 equiv) at −40° C. The resulting mixture was stirred for 1 h at −40° C. The reaction was monitored by LCMS. The reaction was quenched by the addition of NaHCO₃(aq.) (1.00 mL) at −40° C. The resulting mixture was concentrated under vacuum. The residue was purified by silica gel column chromatography, eluted with MeOH/DCM (3%) to afford crude product. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 22% B to 46% B in 9 min, 46% B; Wave Length: 254/220 nm; RT1(min): 8.85; Number Of Runs: 0) to afford N-[(2R)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]prop-2-enamide (32.7 mg, 28.92%) as a white solid.

LC-MS: (M+H)⁺ found: 490.05.

¹H NMR (400 MHz, Chloroform-d) δ 10.90 (s, 1H), 8.53 (s, 1H), 8.13 (s, 1H), 7.77 (s, 1H), 7.39 (d, 1H), 6.72 (d, 1H), 6.61 (t, 1H), 6.36-6.14 (m, 4H), 5.74 (d, 1H), 5.25 (s, 1H), 4.82-4.76 (m, 1H), 4.07 (s, 3H), 3.62-3.61 (m, 2H), 3.30-3.18 (m, 2H), 1.66 (d, 3H).

### Example 116. N-[(2S)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]prop-2-enamide (compound 376)

### 116.1. Synthesis of tert-butyl N-[(2S)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]carbamate

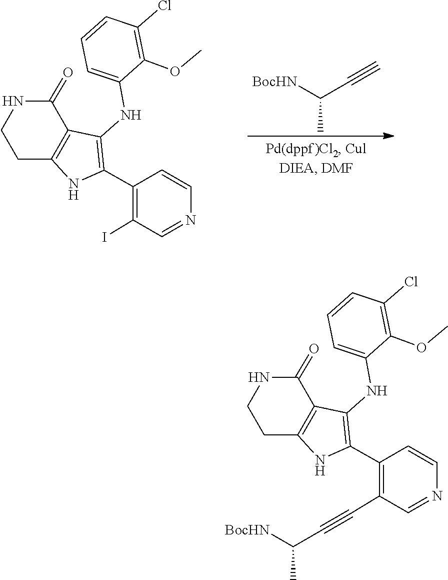 [SMILES: COc1c(Cl)cccc1Nc1c(-c2ccncc2I)[nH]c2c1C(=O)NCC2 ; C#C[C@H](C)NC(=O)OC(C)(C)C ; COc1c(Cl)cccc1Nc1c(-c2ccncc2C#C[C@H](C)NC(=O)OC(C)(C)C)[nH]c2c1C(=O)NCC2]

To a stirred solution of 3-[(3-chloro-2-methoxyphenyl)amino]-2-(3-iodopyridin-4-yl)-1H,5H,6H,7H-pyrrolo[3,2-c]pyridin-4-one (300 mg, 0.61 mmol, 1.00 equiv) in DMF (5.00 mL) was added CuI (58 mg, 0.30 mmol, 0.50 equiv), Pd(dppf)Cl₂CH₂Cl₂ (124 mg, 0.15 mmol, 0.25 equiv), tert-butyl N-[(2S)-but-3-yn-2-yl]carbamate (205 mg, 1.21 mmol, 2.00 equiv) and DIEA (236 mg, 1.82 mmol, 3.00 equiv) at room temperature. The mixture was stirred for 1 h at 50° C. under argon atmosphere. The reaction was monitored by LCMS. The resulting mixture was filtered, the filter cake was washed with CH₂Cl₂: MeOH (3×5 mL). The filtrate was concentrated under reduced pressure. The residue was purified by reverse flash chromatography with the following conditions: column, C18 spherical column; mobile phase, ACN in water, 10% to 70% gradient in 25 min; detector, UV 254 nm to afford crude product. The crude was purified by silica gel column chromatography, eluted with CH₂Cl₂/MeOH (97:3) to afford tert-butyl N-[(2S)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]carbamate (110 mg, 33.84%) as a yellow solid.

LC-MS: (M+H)⁺ found: 536.10.

### 116.2. Synthesis of 2-{3-[(3S)-3-aminobut-1-yn-1-yl]pyridin-4-yl}-3-[(3-chloro-2-methoxyphenyl)amino]-1H,5H,6H,7H-pyrrolo[3,2-c]pyridin-4-one

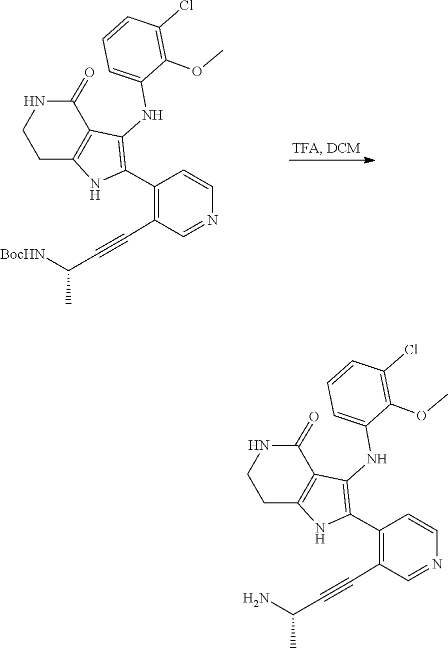 [SMILES: COc1c(Cl)cccc1Nc1c(-c2ccncc2C#C[C@H](C)NC(=O)OC(C)(C)C)[nH]c2c1C(=O)NCC2 ; COc1c(Cl)cccc1Nc1c(-c2ccncc2C#C[C@H](C)N)[nH]c2c1C(=O)NCC2]

To a stirred solution of tert-butyl N-[(2S)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]carbamate (110 mg, 0.19 mmol, 1.00 equiv) in DCM (3.00 mL) was added TFA (1.00 mL) dropwise at 0° C. The mixture was stirred for 1 h at room temperature under nitrogen atmosphere. The reaction was monitored by LCMS. The resulting mixture was concentrated under reduced nitrogen atmosphere. The residue was purified by reverse flash chromatography with the following conditions: column, C18 spherical column; mobile phase, ACN in water, 10% to 60% gradient in 25 min; detector, UV 254 nm to afford 2-{3-[(3S)-3-aminobut-1-yn-1-yl]pyridin-4-yl}-3-[(3-chloro-2-methoxyphenyl)amino]-1H,5H,6H,7H-pyrrolo[3,2-c]pyridin-4-one (85 mg, 95.12%) as a yellow solid.

LC-MS: (M+H)⁺ found: 435.95.

### 116.3. Synthesis of N-[(2S)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]prop-2-enamide

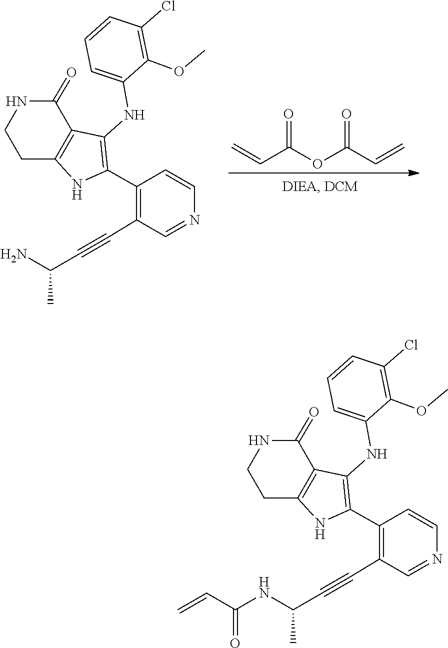

To a stirred solution of 2-{3-[(3S)-3-aminobut-1-yn-1-yl]pyridin-4-yl}-3-[(3-chloro-2-methoxyphenyl)amino]-1H,5H,6H,7H-pyrrolo[3,2-c]pyridin-4-one (80 mg, 0.18 mmol, 1.00 equiv) in DCM (2.00 mL) was added DIEA (119 mg, 0.92 mmol, 5.00 equiv) at room temperature. Prop-2-enoyl prop-2-enoate (46 mg, 0.37 mmol, 2.00 equiv) was added dropwise at −40° C. The mixture was stirred for 0.5 h at −40° C. under nitrogen atmosphere. The reaction was monitored by LCMS. The resulting mixture was extracted with CH₂Cl₂:MeOH (3×20 mL). The combined organic layers were dried over anhydrous Na₂SO₄. After filtration, the filtrate was concentrated under reduced pressure. The crude product (90 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 42% B in 10 min, 42% B; Wave Length: 254/220 nm; RT1(min): 9; Number Of Runs: 0) to afford N-[(2S)-4-(4-{3-[(3-chloro-2-methoxyphenyl)amino]-4-oxo-1H,5H,6H,7H-pyrrolo[3,2-c]pyridin-2-yl}pyridin-3-yl)but-3-yn-2-yl]prop-2-enamide (35.6 mg, 39.27%) as a light yellow solid.

LC-MS: (M+H)⁺ found: 490.05.

¹H NMR (400 MHz, Chloroform-d): δ 10.94 (s, 1H), 8.52 (s, 1H), 8.12 (d, 1H), 7.81 (s, 1H), 7.38 (d, 1H), 6.72 (d, 1H), 6.61 (t, 1H), 6.37-6.14 (m, 4H), 5.76 (d, 1H), 5.25 (s, 1H), 4.82-4.76 (m, 1H), 4.07 (s, 3H), 3.65-3.59 (m, 2H), 3.30-3.23 (m, 2H), 1.66 (d, 3H).

### Example 117. N-[(2S)-4-(4-{3-[(3-fluoro-2-methoxyphenyl)amino]-4-oxo-1H,5H,6H,7H-pyrrolo[3,2-c]pyridin-2-yl}pyridin-3-yl)but-3-yn-2-yl]prop-2-enamide (compound 375)

### 117.1. Synthesis of tert-butyl N-[(2S)-4-(4-{3-[(3-fluoro-2-methoxyphenyl)amino]-4-oxo-1H,5H,6H,7H-pyrrolo[3,2-c]pyridin-2-yl}pyridin-3-yl)but-3-yn-2-yl]carbamate

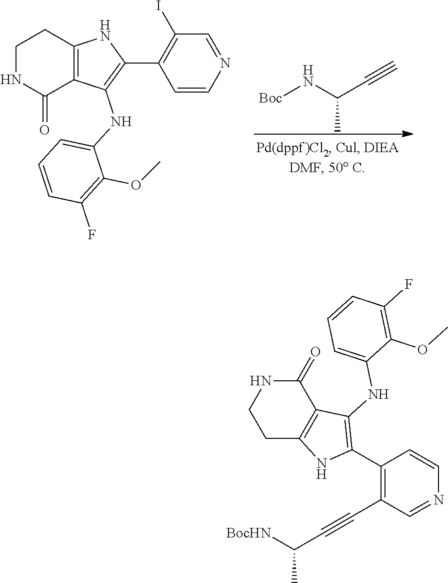 [SMILES: COc1c(F)cccc1Nc1c(-c2ccncc2I)[nH]c2c1C(=O)NCC2 ; C#C[C@H](C)NC(=O)OC(C)(C)C ; COc1c(F)cccc1Nc1c(-c2ccncc2C#C[C@H](C)NC(=O)OC(C)(C)C)[nH]c2c1C(=O)NCC2]

To a stirred solution of 3-[(3-fluoro-2-methoxyphenyl)amino]-2-(3-iodopyridin-4-yl)-1H,5H,6H,7H-pyrrolo[3,2-c]pyridin-4-one (250 mg, 0.52 mmol, 1.00 equiv) and Pd(dppf)C12-CH₂Cl₂ (106 mg, 0.13 mmol, 0.25 equiv) and CuI (49 mg, 0.26 mmol, 0.50 equiv) in DMF (3 mL) were added tert-butyl N-[(2S)-but-3-yn-2-yl]carbamate (221 mg, 1.30 mmol, 2.50 equiv) and DIEA (337 mg, 2.61 mmol, 5.00 equiv) at room temperature under Ar atmosphere. The resulting mixture was stirred for 2 h at 50° C. under Ar atmosphere in a sealed tube. The resulting mixture was concentrated under reduced pressure. The residue was purified by reverse flash chromatography with the following conditions (column, C18 spherical column; mobile phase, MeCN in water, 10% to 60% gradient in 30 min; detector, UV 254 nm) to afford tert-butyl N-[(2S)-4-(4-{3-[(3-fluoro-2-methoxyphenyl)amino]-4-oxo-1H,5H,6H,7H-pyrrolo[3,2-c]pyridin-2-yl}pyridin-3-yl)but-3-yn-2-yl]carbamate (195 mg, 71.80%) as a yellow solid.

LC-MS: (M+H)⁺ found: 520.20.

### 117.2. Synthesis of 2-{3-[(3S)-3-aminobut-1-yn-1-yl]pyridin-4-yl}-3-[(3-fluoro-2-methoxyphenyl)amino]-1H,5H,6H,7H-pyrrolo[3,2-c]pyridin-4-one

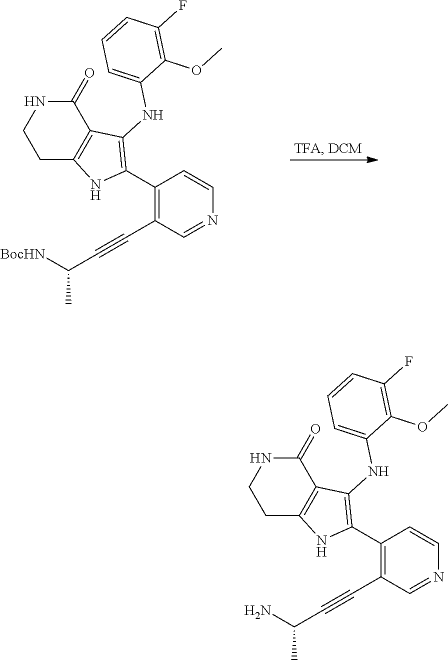 [SMILES: COc1c(F)cccc1Nc1c(-c2ccncc2C#C[C@H](C)NC(=O)OC(C)(C)C)[nH]c2c1C(=O)NCC2 ; COc1c(F)cccc1Nc1c(-c2ccncc2C#C[C@H](C)N)[nH]c2c1C(=O)NCC2]

To a stirred solution of tert-butyl N-[(2S)-4-(4-{3-[(3-fluoro-2-methoxyphenyl)amino]-4-oxo-1H,5H,6H,7H-pyrrolo[3,2-c]pyridin-2-yl}pyridin-3-yl)but-3-yn-2-yl]carbamate (170 mg, 0.32 mmol, 1.00 equiv) in DCM (2.00 mL) was added TFA (2.00 mL) dropwise at 0° C. The resulting mixture was stirred for 2 h at room temperature. The resulting mixture was worked up and dried using nitrogen. The residue was purified by reverse flash chromatography with the following conditions (column, C18 spherical column; mobile phase, MeOH in water, 10% to 50% gradient in 20 min; detector, UV 254 nm) to afford 2-{3-[(3S)-3-aminobut-1-yn-1-yl]pyridin-4-yl}-3-[(3-fluoro-2-methoxyphenyl)amino]-1H,5H,6H,7H-pyrrolo[3,2-c]pyridin-4-one (75 mg, 54.65%) as a yellow solid.

LC-MS: (M+H)⁺ found: 420.05.

### 117.3. Synthesis of N-[(2S)-4-(4-{3-[(3-fluoro-2-methoxyphenyl)amino]-4-oxo-1H,5H,6H,7H-pyrrolo[3,2-c]pyridin-2-yl}pyridin-3-yl)but-3-yn-2-yl]prop-2-enamide

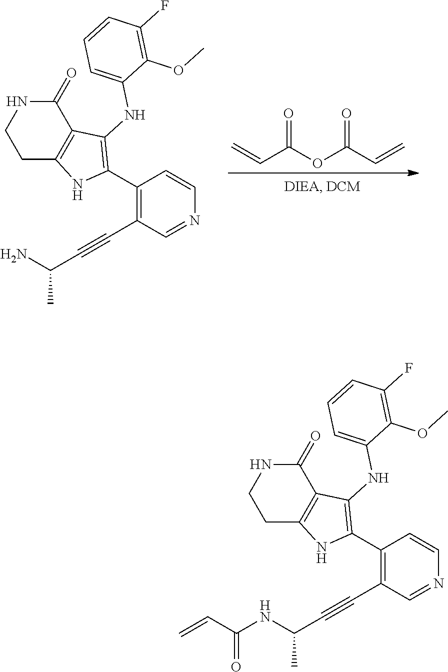

To a stirred solution of 2-{3-[(3S)-3-aminobut-1-yn-1-yl]pyridin-4-yl}-3-[(3-fluoro-2-methoxyphenyl)amino]-1H,5H,6H,7H-pyrrolo[3,2-c]pyridin-4-one (75 mg, 0.17 mmol, 1.00 equiv) and DIEA (115 mg, 0.89 mmol, 5.00 equiv) in THF was added prop-2-enoyl prop-2-enoate (33 mg, 0.26 mmol, 1.50 equiv) dropwise at 0° C. The resulting mixture was stirred for 2 h at room temperature. The resulting mixture was extracted with EtOAc (3×5 mL). The combined organic layers were washed with brine (1×5 mL), dried over anhydrous Na₂SO₄. After filtration, the filtrate was concentrated under reduced pressure. The residue was purified by silica gel column chromatography, eluted with CH₂Cl₂/MeOH (10:1) to afford N-[(2S)-4-(4-{3-[(3-fluoro-2-methoxyphenyl)amino]-4-oxo-1H,5H,6H,7H-pyrrolo[3,2-c]pyridin-2-yl}pyridin-3-yl)but-3-yn-2-yl]prop-2-enamide (80 mg) as a yellow oil. The crude product (80 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 19% B to 44% B in 9 min, 44% B; Wave Length: 254/220 nm; RT1(min): 8.85; Number Of Runs: 0) to afford N-[(2S)-4-(4-{3-[(3-fluoro-2-methoxyphenyl)amino]-4-oxo-1H,5H,6H,7H-pyrrolo[3,2-c]pyridin-2-yl}pyridin-3-yl)but-3-yn-2-yl]prop-2-enamide (22.5 mg, 26.23%) as a orange solid.

LC-MS: (M+H)⁺ found: 474.2

¹H NMR (300 MHz, Chloroform-d) δ 10.85 (s, 1H), 8.55 (s, 1H), 8.16 (s, 1H), 7.72 (s, 1H), 7.45 (s, 1H), 6.61-6.43 (m, 2H), 6.37-6.31 (m, 1H), 6.20-6.06 (m, 3H), 5.76-5.72 (m, 1H), 5.21 (s, 1H), 4.81-4.77 (m, 1H), 4.10 (s, 3H), 3.61-3.59 (m, 2H), 3.25-3.20 (m, 2H), 1.66 (d, 3H)

### Example 118. N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]prop-2-enamide (compound 492)

### 118.1. Synthesis of tert-butyl N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]carbamate

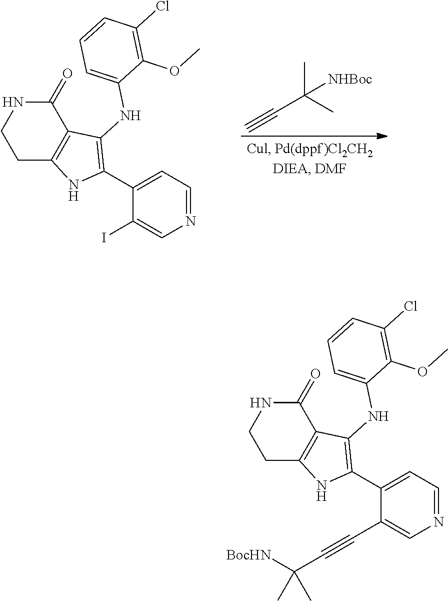 [SMILES: COc1c(Cl)cccc1Nc1c(-c2ccncc2I)[nH]c2c1C(=O)NCC2 ; C#CC(C)(C)NC(=O)OC(C)(C)C ; COc1c(Cl)cccc1Nc1c(-c2ccncc2C#CC(C)(C)NC(=O)OC(C)(C)C)[nH]c2c1C(=O)NCC2]

To a stirred mixture of 3-[(3-chloro-2-methoxyphenyl)amino]-2-(3-iodopyridin-4-yl)-1H,5H,6H,7H-pyrrolo[3,2-c]pyridin-4-one (150 mg, 0.30 mmol, 1.00 equiv) and tert-butyl N-(2-methylbut-3-yn-2-yl)carbamate (111 mg, 0.60 mmol, 2.00 equiv) in DMF (3.00 mL) were added Pd(dppf)Cl₂CH₂Cl₂ (123 mg, 0.15 mmol, 0.50 equiv) and CuI (28 mg, 0.15 mmol, 0.50 equiv) and DIEA (235 mg, 1.81 mmol, 6.00 equiv) at room temperature under nitrogen atmosphere. The resulting suspension was backfilled with argon three times and stirred for 2h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by reverse flash chromatography with the following conditions: column, silica gel; mobile phase, MeCN in water, 0% to 100% gradient in 50 min; detector, UV 254 nm. To afford tert-butyl N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]carbamate (190 mg, 96.0%) as a yellow solid.

LC-MS: (M+H)⁺ found 550.1.

### 118.2. Synthesis of 2-[3-(3-amino-3-methylbut-1-yn-1-yl)pyridin-4-yl]-3-[(3-chloro-2-methoxyphenyl)amino]-1H,5H,6H,7H-pyrrolo[3,2-c]pyridin-4-one

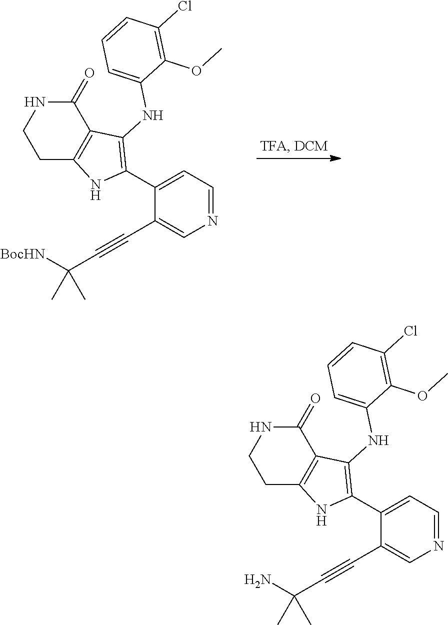 [SMILES: COc1c(Cl)cccc1Nc1c(-c2ccncc2C#CC(C)(C)NC(=O)OC(C)(C)C)[nH]c2c1C(=O)NCC2 ; COc1c(Cl)cccc1Nc1c(-c2ccncc2C#CC(C)(C)N)[nH]c2c1C(=O)NCC2]

To a stirred mixture of tert-butyl N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]carbamate (150 mg, 0.27 mmol, 1.00 equiv) in DCM (3 mL) was added TFA (1 mL) at room temperature under nitrogen atmosphere. The resulting mixture was stirred for 1 h and concentrated under reduced pressure to afford 2-[3-(3-amino-3-methylbut-1-yn-1-yl)pyridin-4-yl]-3-[(3-chloro-2-methoxyphenyl)amino]-1H,5H,6H,7H-pyrrolo[3,2-c]pyridin-4-one (250 mg, crude) as a red oil.

LC-MS: (M+H)⁺ found 450.

### 118.3. Synthesis of N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]prop-2-enamide

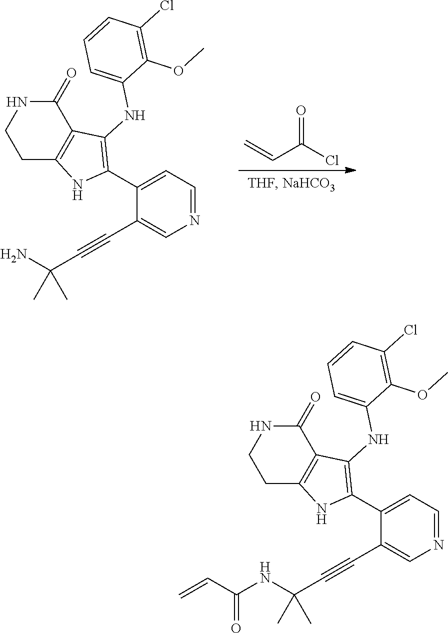

To a stirred solution of 2-[3-(3-amino-3-methylbut-1-yn-1-yl)pyridin-4-yl]-3-[(3-chloro-2-methoxyphenyl)amino]-1H,5H,6H,7H-pyrrolo[3,2-c]pyridin-4-one (80 mg, 0.17 mmol, 1.00 equiv) in THF (1.50 mL) and NaHCO₃(sat.)(1.50 mL) was added acryloyl chloride 5 (18 mg, 0.20 mmol, 1.15 equiv) at 0° C. under nitrogen atmosphere. The resulting mixture was stirred for 1 h at 0° C. under nitrogen atmosphere. The reaction was monitored by LCMS. LCMS showed the reaction was completed. The resulting mixture was extracted with EtOAc (3×15 mL), dried over anhydrous Na₂SO₄. After filtration, the filtrate was concentrated under reduced pressure. The resulting mixture was concentrated under reduced pressure. The crude product was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 32% B to 46% B in 9 min, 46% B; Wave Length: 254/220 nm; RT1(min): 9.78; Number Of Runs: 0) to afford N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]prop-2-enamide (13.70 mg, 15.21%) as a yellow solid.

LC-MS: (M+H)⁺ found 503.95.

¹H NMR (300 MHz, DMSO-d6) δ 11.65 (s, 1H), 8.75 (s, 1H), 8.51 (s, 1H), 8.23 (d, 1H), 7.65 (s, 1H), 7.35 (d, 1H), 7.21 (s, 1H), 6.70 (d, 2H), 6.34-6.32 (m, 1H), 6.29-6.24 (m, 1H), 6.14 (t, 1H), 5.71-5.67 (m, 1H), 3.89 (s, 3H), 3.45-3.42 (m, 2.4 Hz, 2H), 3.02 (t, 2H), 1.64 (s, 6H).

### Example 119. 3-[(3-chloro-2-methoxyphenyl)amino]-2-(3-{2-[(1S,3R,5S)-2-(prop-2-enoyl)-2-azabicyclo[3.1.0]hexan-3-yl]ethynyl}pyridin-4-yl)-1H,5H,6H,7H-pyrrolo[3,2-c]pyridin-4-one (compound 517)

### 119.1. Synthesis of tert-butyl (1S,3R,5S)-3-(hydroxymethyl)-2-azabicyclo[3.1.0]hexane-2-carboxylate

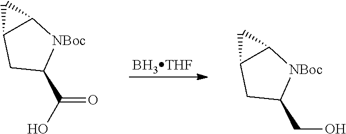 [SMILES: CC(C)(C)OC(=O)N1[C@@H](C(=O)O)C[C@@H]2C[C@@H]21 ; CC(C)(C)OC(=O)N1[C@@H](CO)C[C@@H]2C[C@@H]21 ; "BH3·THF"]

To a stirred solution of (1S,3R,5S)-2-(tert-butoxycarbonyl)-2-azabicyclo[3.1.0]hexane-3-carboxylic acid (1 g, 4.40 mmol, 1.00 equiv) in THF (10 mL) was added BH₃(1M in THF, 8.80 mL, 8.80 mmol, 2.00 equiv) dropwise at 0° C. under nitrogen atmosphere. The resulting mixture was stirred for 2 h at room temperature under nitrogen atmosphere. The reaction was quenched by the addition of MeOH (5 mL) at 0° C. The resulting mixture was concentrated under reduced pressure to afford tert-butyl (1S,3R,5S)-3-(hydroxymethyl)-2-azabicyclo[3.1.0]hexane-2-carboxylate (1.1 g crude) as a colorless oil.

LC-MS: (M+H−56)⁺ found: 157.95

### 119.2. Synthesis of tert-butyl (1S,3R,5S)-3-formyl-2-azabicyclo[3.1.0]hexane-2-carboxylate

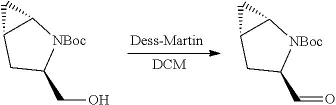 [SMILES: CC(C)(C)OC(=O)N1[C@@H](CO)C[C@@H]2C[C@@H]21 ; CC(C)(C)OC(=O)N1[C@@H](C=O)C[C@@H]2C[C@@H]21]

To a stirred mixture of tert-butyl (1S,3R,5S)-3-(hydroxymethyl)-2-azabicyclo[3.1.0]hexane-2-carboxylate (0.97 g, 4.54 mmol, 1.00 equiv) in DCM (20 mL) was added Dess-Martin (2.31 g, 5.45 mmol, 1.20 equiv) dropwise at 0° C. under nitrogen atmosphere. The resulting mixture was stirred for 2 h at room temperature under nitrogen atmosphere. The reaction was quenched by the addition of sat. Na₂SO₃(aq.) (5 mL) at room temperature. The reaction was added Na₂CO₃(sat.) to adjust PH to 7-8. The resulting mixture was extracted with CH₂Cl₂ (3×20 mL), dried over anhydrous Na₂SO₄. After filtration, the filtrate was concentrated under reduced pressure. The residue was purified by silica gel column chromatography, eluted with PE/EA (5:1) to afford tert-butyl (1S,3R,5S)-3-formyl-2-azabicyclo[3.1.0]hexane-2-carboxylate as a colorless oil.

¹H-NMR (300 MHz, Chloroform-d) δ 9.50 (s, 1H), 4.01-3.21 (m, 2H), 2.34-2.01 (m, 2H), 1.59-1.52 (m, 1H), 1.46 (s, 9H), 0.80-0.70 (m, 1H), 0.54 (s, 1H).

### 119.3. Synthesis of tert-butyl (1S,3R,5S)-3-ethynyl-2-azabicyclo[3.1.0]hexane-2-carboxylate

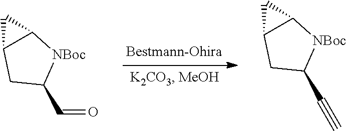

To a stirred solution of tert-butyl (1S,3R,5S)-3-formyl-2-azabicyclo[3.1.0]hexane-2-carboxylate (520 mg, 2.46 mmol, 1.00 equiv) and K₂CO₃ (680 mg, 4.92 mmol, 2.00 equiv) in methanol (10.00 mL) was added Bestmann-Ohira (567 mg, 2.95 mmol, 1.20 equiv) dropwise at 0° C. under nitrogen atmosphere. The resulting mixture was stirred for 2 h at room temperature under nitrogen atmosphere. The reaction was quenched by the addition of potassium sodium tartrate (sat.) (5 mL) at room temperature. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EA (5:1) to afford tert-butyl (1S,3R,5S)-3-ethynyl-2-azabicyclo[3.1.0]hexane-2-carboxylate (470 mg, 92.12%) as a white oil.

¹H-NMR (400 MHz, Chloroform-d) δ 4.24 (t, 1H), 3.27 (s, 1H), 2.38-2.12 (m, 3H), 1.67-1.49 (m, 1H), 1.42 (s, 9H), 0.84-0.70 (m, 1H), 0.42-0.26 (m, 1H).

### 119.4. Synthesis of tert-butyl (1R,3S,5R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate

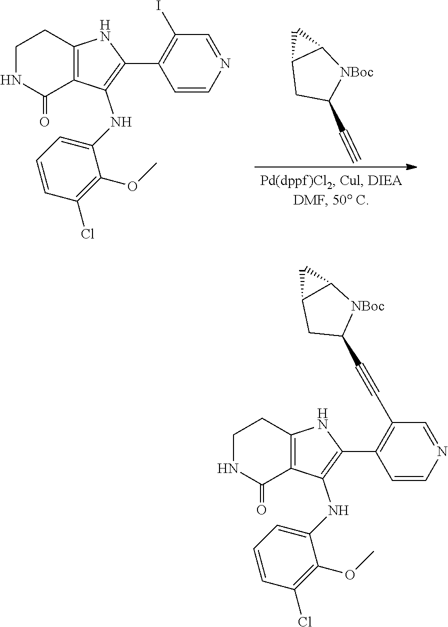 [SMILES: COc1c(Cl)cccc1Nc1c(-c2ccncc2I)[nH]c2c1C(=O)NCC2 ; C#C[C@H]1C[C@@H]2C[C@@H]2N1C(=O)OC(C)(C)C ; COc1c(Cl)cccc1Nc1c(-c2ccncc2C#C[C@H]2C[C@@H]3C[C@@H]3N2C(=O)OC(C)(C)C)[nH]c2c1C(=O)NCC2]

To a stirred mixture of 3-[(3-chloro-2-methoxyphenyl)amino]-2-(3-iodopyridin-4-yl)-1H,5H,6H,7H-pyrrolo[3,2-c]pyridin-4-one (150 mg, 0.30 mmol, 1.00 equiv) and Pd(dppf)Cl₂CH₂Cl₂ (61 mg, 0.07 mmol, 0.25 equiv) in DMF (2 mL) was added tert-butyl (1R,3S,5R)-3-ethynyl-2-azabicyclo[3.1.0]hexane-2-carboxylate (25 mg, 0.12 mmol, 3.00 equiv) and DIEA (195 mg, 1.52 mmol, 5.00 equiv) dropwise at room temperature under argon atmosphere. The resulting mixture was stirred for 2 h at 50° C. under argon atmosphere. The residue was purified by reverse flash chromatography with the following conditions: column, silica gel; mobile phase, MeCN in water, 10% to 70% gradient in 20 min; detector, UV 254 nm. This resulted in tert-butyl (1R,3S,5R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (170 mg, 97.67%) as a yellow solid.

LC-MS: (M+H)⁺ found: 574.05.

### 119.5. Synthesis of 2-(3-{2-[(1S,3R,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one

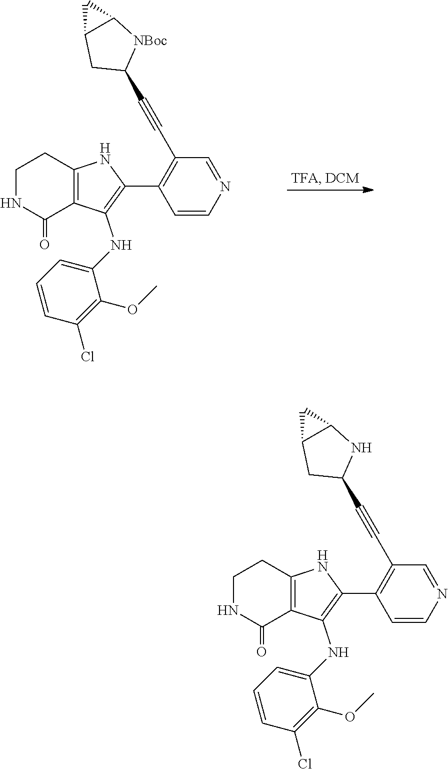 [SMILES: COc1c(Cl)cccc1Nc1c(-c2ccncc2C#C[C@H]2C[C@@H]3C[C@@H]3N2C(=O)OC(C)(C)C)[nH]c2c1C(=O)NCC2 ; COc1c(Cl)cccc1Nc1c(-c2ccncc2C#C[C@H]2C[C@@H]3C[C@@H]3N2)[nH]c2c1C(=O)NCC2]

To a stirred mixture of tert-butyl (1S,3R,5S)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (120 mg, 0.04 mmol, 1.00 equiv) in 5 DCM (3.00 mL) was added TFA (1.00 mL) at room temperature under nitrogen atmosphere. The resulting mixture was stirred for 1 h and concentrated under reduced pressure to afford 2-(3-{2-[(1S,3R,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one (160 mg, crude) as a red oil.

LC-MS: (M+H)⁺ found: 474.05

### 119.6. Synthesis of 3-[(3-chloro-2-methoxyphenyl)amino]-2-(3-{2-[(1S,3R,5S)-2-(prop-2-enoyl)-2-azabicyclo[3.1.0]hexan-3-yl]ethynyl}pyridin-4-yl)-1H,5H,6H,7H-pyrrolo[3,2-c]pyridin-4-one

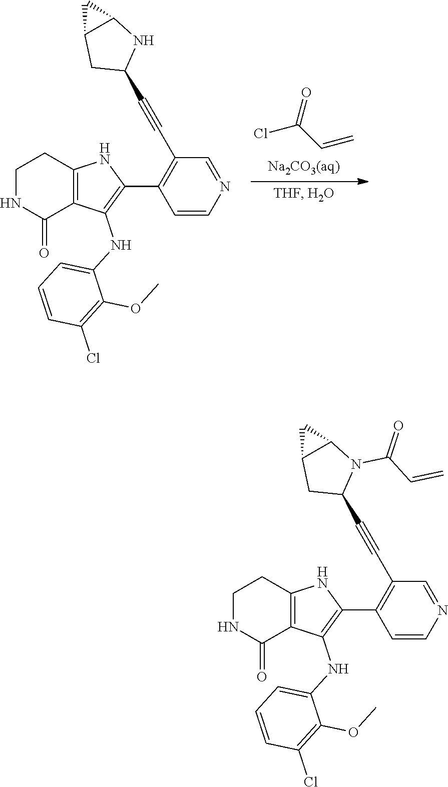

To a stirred solution of 2-(3-{2-[(1S,3R,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one (100 mg, 0.21 mmol, 1.00 equiv) and NaHCO₃(sat.) (1.00 mL) in THF (1.00 mL) was added acryloyl chloride (19 mg, 0.21 mmol, 1.00 equiv) dropwise at room temperature under nitrogen atmosphere. The resulting mixture was stirred for 1 h at room temperature under nitrogen atmosphere. The resulting mixture was extracted with CH₂Cl₂/MeOH(10/1) (3×10 mL). The combined organic layers were washed with brine (3×10 mL), dried over anhydrous Na₂SO₄. After filtration, the filtrate was concentrated under reduced pressure. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 28% B to 50% B in 9 min, 50% B; Wave Length: 254/220 nm; RT1(min): 8.85; Number Of Runs: 0) to afford 3-[(3-chloro-2-methoxyphenyl)amino]-2-(3-{2-[(1S,3R,5S)-2-(prop-2-enoyl)-2-azabicyclo[3.1.0]hexan-3-yl]ethynyl}pyridin-4-yl)-1H,5H,6H,7H-pyrrolo[3,2-c]pyridin-4-one (10.8 mg, 9.55%) as a yellow solid.

LC-MS: (M+H)⁺ found: 527.90

¹H NMR (400 MHz, Chloroform-d) δ 11.00 (s, 1H), 8.59 (s, 1H), 8.16 (s, 1H), 7.74 (s, 1H), 7.44 (s, 1H), 6.84-6.65 (m, 2H), 6.60 (t, 1H), 6.48-6.33 (m, 1H), 6.24-6.14 (m, 1H), 5.88-5.69 (m, 1H), 5.22 (s, 1H), 4.89-4.73 (m, 1H), 4.07 (s, 3H), 3.67-3.52 (m, 3H), 3.30-3.20 (m, 2H), 2.66-2.45 (m, 2H), 2.06-1.95 (m, 1H), 1.13-1.01 (m, 1H), 0.70-0.60 (m, 1H).

### Example 120. 3-[(3-chloro-2-methoxyphenyl)amino]-2-(3-{2-[(1R,3R,5R)-2-(prop-2-enoyl)-2-azabicyclo[3.1.0]hexan-3-yl]ethynyl}pyridin-4-yl)-1H,5H,6H,7H-pyrrolo[3,2-c]pyridin-4-one (compound 518)

### 120.1. Synthesis of tert-butyl (1R,3R,5R)-3-(hydroxymethyl)-2-azabicyclo[3.1.0]hexane-2-carboxylate

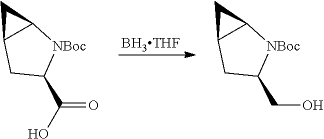 [SMILES: CC(C)(C)OC(=O)N1[C@@H](C(=O)O)C[C@H]2C[C@H]21 ; CC(C)(C)OC(=O)N1[C@@H](CO)C[C@H]2C[C@H]21 ; "BH3·THF"]

To a stirred solution of (1R,3R,5R)-2-(tert-butoxycarbonyl)-2-azabicyclo[3.1.0]hexane-3-carboxylic acid (1.0 g, 4.40 mmol, 1.00 equiv) in THF (20.00 mL) was added BH₃ (1M in THF, 6.6 mL, 6.60 mmol, 1.50 equiv) dropwise at 0° C. under nitrogen atmosphere. The mixture was stirred for 1 h at 70° C. under nitrogen atmosphere. The reaction was monitored by LCMS. Desired product could be detected by LCMS. The reaction was quenched by the addition of MeOH (3 mL) at 0° C. The resulting mixture was concentrated under reduced pressure to obtain tert-butyl (1R,3R,5R)-3-(hydroxymethyl)-2-azabicyclo[3.1.0]hexane-2-carboxylate (1.2 g, crude) as a colorless oil.

LC-MS: (M+H)⁺ found: 157.95.

### 120.2. Synthesis of tert-butyl (1R,3R,5R)-3-formyl-2-azabicyclo[3.1.0]hexane-2-carboxylate

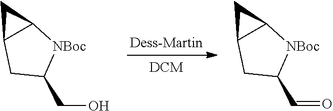 [SMILES: CC(C)(C)OC(=O)N1[C@@H](CO)C[C@H]2C[C@H]21 ; CC(C)(C)OC(=O)N1[C@@H]2C[C@@H]2C[C@@H]1C=O]

To a stirred solution of tert-butyl (1R,3R,5R)-3-(hydroxymethyl)-2-azabicyclo[3.1.0]hexane-2-carboxylate (900 mg, 4.22 mmol, 1.00 equiv) in DCM (20.00 mL) was added Dess-Martin (2.15 g, 5.06 mmol, 1.2 equiv) at 0° C. The mixture was stirred for 2h at room temperature under nitrogen atmosphere. The reaction was monitored by LCMS. The reaction was quenched by the addition of sat. Na₂SO₃ (aq.) (2 mL) at room temperature. The mixture was neutralized to pH 7 with saturated NaHCO₃(aq.). The resulting mixture was extracted with EtOAc (3×20 mL). The combined organic layers were dried over anhydrous Na₂SO₄. After filtration, the filtrate was concentrated under reduced pressure. The residue was purified by silica gel column chromatography, eluted with PE/EA (10:1) to afford tert-butyl (1R,3R,5R)-3-formyl-2-azabicyclo[3.1.0]hexane-2-carboxylate (620 mg, 69.55%) as a colorless oil.

LC-MS: (M+H)⁺ found: 155.95.

### 120.3. Synthesis of tert-butyl (1R,3R,5R)-3-ethynyl-2-azabicyclo[3.1.0]hexane-2-carboxylate

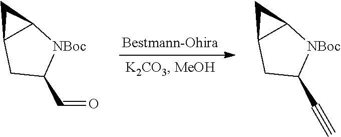

To a stirred solution of tert-butyl (1R,3R,5R)-3-formyl-2-azabicyclo[3.1.0]hexane-2-carboxylate (500 mg, 2.37 mmol, 1.00 equiv) in MeOH (5.00 mL) was added K₂CO₃ (981 mg, 7.10 mmol, 3.00 equiv) and Bestmann-Ohira (682 mg, 3.55 mmol, 1.50 equiv) at 0° C. The mixture was stirred for 2 h at 0° C. under nitrogen atmosphere. The reaction was quenched by the addition of potassium sodium tartrate (sat.) (5 mL) at room temperature. The resulting mixture was extracted with EtOAc (3×20 mL). The combined organic layers were dried over anhydrous Na₂SO₄. After filtration, the filtrate was concentrated under reduced pressure. The residue was purified by silica gel column chromatography, eluted with PE/EA (10:1) to afford tert-butyl (1R,3R,5R)-3-ethynyl-2-azabicyclo[3.1.0]hexane-2-carboxylate (420 mg, 85.62%) as a colorless oil.

LC-MS: (M+H)⁺ found: 152.3.

### 120.4. Synthesis of tert-butyl (1R,3R,5R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate

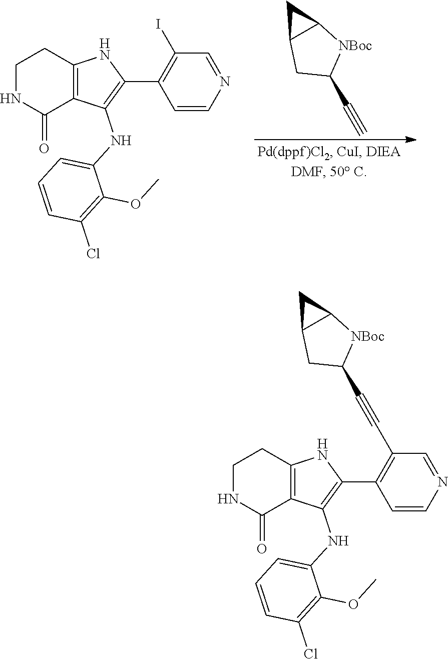 [SMILES: COc1c(Cl)cccc1Nc1c(-c2ccncc2I)[nH]c2c1C(=O)NCC2 ; C#C[C@H]1C[C@H]2C[C@H]2N1C(=O)OC(C)(C)C ; COc1c(Cl)cccc1Nc1c(-c2ccncc2C#C[C@H]2C[C@H]3C[C@H]3N2C(=O)OC(C)(C)C)[nH]c2c1C(=O)NCC2]

To a stirred solution of 3-[(3-chloro-2-methoxyphenyl)amino]-2-(3-iodopyridin-4-yl)-1H,5H,6H,7H-pyrrolo[3,2-c]pyridin-4-one (100 mg, 0.20 mmol, 1.00 equiv) in DMF (2.00 mL) was added tert-butyl (1R,3R,5R)-3-ethynyl-2-azabicyclo[3.1.0]hexane-2-carboxylate (105 mg, 0.51 mmol, 2.50 equiv), CuI (19 mg, 0.10 mmol, 0.50 equiv), Pd(dppf)Cl₂·CH₂Cl₂ (82 mg, 0.10 mmol, 0.50 equiv) and DIAD (123 mg, 0.61 mmol, 3.00 equiv) at room temperature. The mixture was stirred for 2h at 50° C. under argon atmosphere. The reaction was monitored by LCMS. The residue was purified by reverse flash chromatography with the following conditions: column C18 silica gel; mobile phase, MeCN in water, 10% to 50% gradient in 10 min; detector, UV 254 nm. This resulted in tert-butyl (1R,3R,5R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (100 mg, 86.18%) as a yellow solid.

LC-MS: (M+H)⁺ found: 574.15.

### 120.5. Synthesis of 2-(3-{2-[(1R,3R,5R)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one

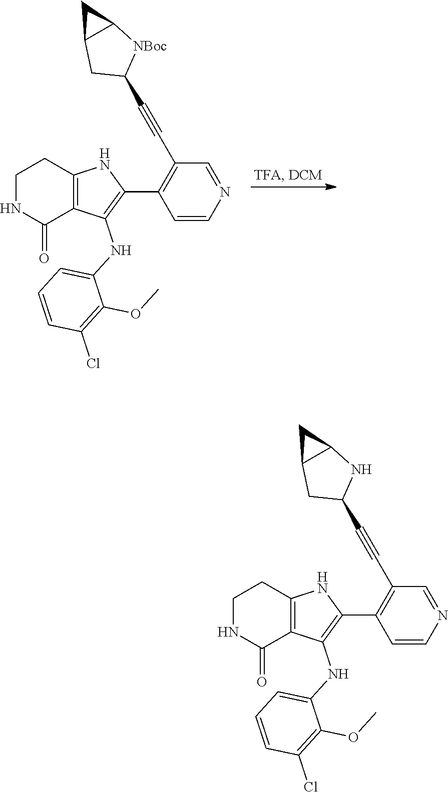 [SMILES: COc1c(Cl)cccc1Nc1c(-c2ccncc2C#C[C@H]2C[C@H]3C[C@H]3N2C(=O)OC(C)(C)C)[nH]c2c1C(=O)NCC2 ; COc1c(Cl)cccc1Nc1c(-c2ccncc2C#C[C@H]2C[C@H]3C[C@H]3N2)[nH]c2c1C(=O)NCC2]

To a stirred mixture of tert-butyl (1R,3R,5R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (90 mg, 0.16 mmol, 1.00 equiv) in DCM (3.00 mL) was added TFA (1.00 mL) at room temperature under nitrogen atmosphere. The resulting mixture was stirred for 1 h and concentrated under reduced pressure to afford 2-(3-{2-[(1R,3R,5R)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one (150 mg, crude) as a red oil.

LC-MS: (M+H)⁺ found: 474.15.

### 120.6. Synthesis of 3-[(3-chloro-2-methoxyphenyl)amino]-2-(3-{2-[(1R,3R,5R)-2-(prop-2-enoyl)-2-azabicyclo[3.1.0]hexan-3-yl]ethynyl}pyridin-4-yl)-1H,5H,6H,7H-pyrrolo[3,2-c]pyridin-4-one

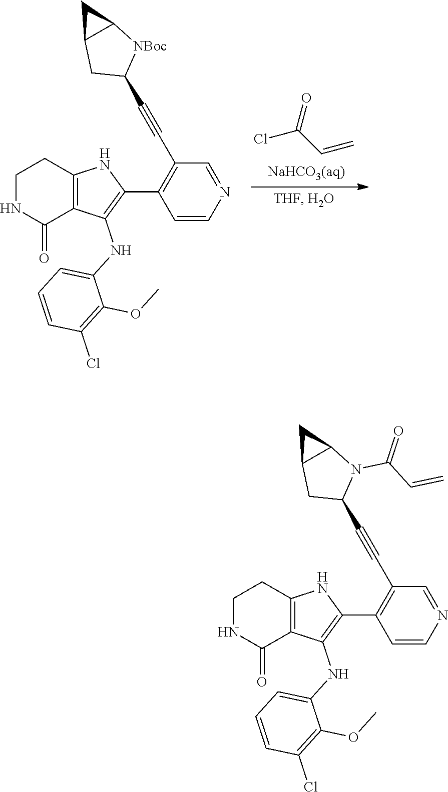

To a stirred solution of 2-(3-{2-[(1R,3R,5R)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one (90 mg, 0.19 mmol, 1.00 equiv) in THF (1.50 mL) was added NaHCO₃(aq.)(1.50 mL) to adjust PH to 9 at 0° C. Acryloyl chloride (17 mg, 0.19 mmol, 1.00 equiv) was added dropwise at 0° C. The mixture was stirred for 0.5 h at 0° C. under nitrogen atmosphere. The reaction was monitored by LCMS. The resulting mixture was extracted with CH₂Cl₂:MeOH (10:1)(3×10 mL). The combined organic layers were dried over anhydrous Na₂SO₄. After filtration, the filtrate was concentrated under reduced pressure. The residue was purified by silica gel column chromatography, eluted with CH₂Cl₂/MeOH (97:3) to afford crude product. The crude product (30 mg) was purified by Prep-HPLC with the following conditions (Column: Xselect CSH F-Phenyl OBD column, 30*250 mm, 5 μm; Mobile Phase A: Water (10 mmol/L NH₄HCO₃), Mobile Phase B: ACN; Flow rate: 60 mL/min; Gradient: 19% B to 49% B in 7 min; Wave Length: 254 nm; RT1(min): 6.5; Number Of Runs: 0) to afford 3-[(3-chloro-2-methoxyphenyl)amino]-2-(3-{2-[(1R,3R,5R)-2-(prop-2-enoyl)-2-azabicyclo[3.1.0]hexan-3-yl]ethynyl}pyridin-4-yl)-1H,5H,6H,7H-pyrrolo[3,2-c]pyridin-4-one (8.2 mg, 8.16%) as a light yellow solid.

LC-MS: (M+H)⁺ found: 528.10.

¹H NMR (400 MHz, Chloroform-d): 611.09 (s, 1H), 8.51 (s, 1H), 8.07 (d, 1H), 7.94 (s, 1H), 7.39 (d, 1H), 6.79-6.76 (m, 2H), 6.62 (t, 1H), 6.46-6.38 (m, 1H), 6.18 (d, 1H), 5.79 (d, 1H), 5.28-5.24 (m, 2H), 4.07 (s, 3H), 3.65-3.56 (m, 3H), 3.31-3.28 (m, 2H), 2.75-2.71 (m, 1H), 2.39-2.35 (m, 1H), 1.94-1.86 (m, 1H), 1.12-1.09 (m, 2H).

### Example 121. 3-[(3-chloro-2-methoxyphenyl)amino]-2-(3-[2-[(2R)-4,4-difluoro-1-(prop-2-enoyl)pyrrolidin-2-yl]ethynyl]pyridin-4-yl)-1H,5H,6H,7H-pyrrolo[3,2-c]pyridin-4-one (compound 519)

### 121.1. Synthesis of tert-butyl (2R)-4,4-difluoro-2-formylpyrrolidine-1-carboxylate

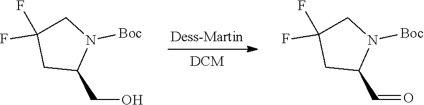 [SMILES: CC(C)(C)OC(=O)N1CC(F)(F)C[C@@H]1CO ; CC(C)(C)OC(=O)N1CC(F)(F)C[C@@H]1C=O]

To a stirred solution of tert-butyl (2R)-4,4-difluoro-2-(hydroxymethyl)pyrrolidine-1-carboxylate (100.00 mg, 0.42 mmol, 1.00 equiv) in DCM (10.00 mL) was added Dess-Martin (214 mg, 0.50 mmol, 1.20 equiv) dropwise at 0° C. under nitrogen atmosphere. The resulting mixture was stirred for 3 h at 0° C. under nitrogen atmosphere. The reaction was quenched by the addition of sat. Na₂SO₃(aq.) (2 mL) at room temperature. The reaction was added Na₂CO₃(sat.) to adjust PH to 7-8. The resulting mixture was extracted with CH₂Cl₂ (3×10 mL), dried over anhydrous Na₂SO₄. After filtration, the filtrate was concentrated under reduced pressure. The residue was purified by silica gel column chromatography, eluted with PE/EtOAc (5:1) to afford tert-butyl (2R)-4,4-difluoro-2-formylpyrrolidine-1-carboxylate (46 mg, 46.39%) as a colorless oil.

LC-MS: (M+H−56)⁺ found: 180.

### 121.2. Synthesis of tert-butyl (2R)-2-ethynyl-4,4-difluoropyrrolidine-1-carboxylate

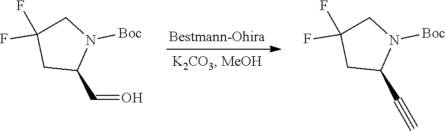 [SMILES: CC(C)(C)OC(=O)N1CC(F)(F)C[C@@H]1C=O ; C#C[C@H]1CC(F)(F)CN1C(=O)OC(C)(C)C]

To a stirred mixture of tert-butyl (2R)-4,4-difluoro-2-formylpyrrolidine-1-carboxylate (100 mg, 0.43 mmol, 1.00 equiv) in MeOH (5 mL) was added K₂CO₃ (117 mg, 0.85 mmol, 2.00 equiv) and Best-Ohria (98 mg, 0.51 mmol, 1.20 equiv) dropwise at 0° C. The resulting mixture was stirred for 3 h at room temperature under nitrogen atmosphere. The reaction was quenched by the addition of potassium sodium tartrate (sat.) (5 mL) at room temperature. The resulting mixture was extracted with EtOAc (3×20 mL). The combined organic layers were dried over anhydrous Na₂SO₄. After filtration, the filtrate was concentrated under reduced pressure. The residue was purified by silica gel column chromatography, eluted with PE/EA (5:1) to afford tert-butyl (2R)-2-ethynyl-4,4-difluoropyrrolidine-1-carboxylate (75 mg, 76.29%) as a yellow oil.

### 121.3. Synthesis of tert-butyl (2R)-2-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]-4,4-difluoropyrrolidine-1-carboxylate

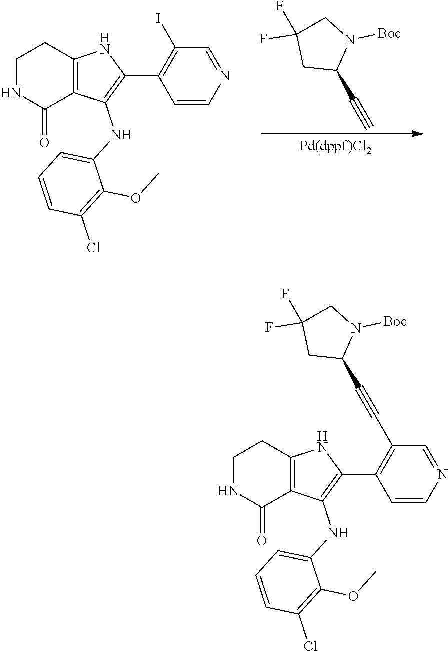 [SMILES: COc1c(Cl)cccc1Nc1c(-c2ccncc2I)[nH]c2c1C(=O)NCC2 ; C#C[C@H]1CC(F)(F)CN1C(=O)OC(C)(C)C ; COc1c(Cl)cccc1Nc1c(-c2ccncc2C#C[C@H]2CC(F)(F)CN2C(=O)OC(C)(C)C)[nH]c2c1C(=O)NCC2]

To a stirred solution of 3-[(3-chloro-2-methoxyphenyl)amino]-2-(pyridin-4-yl)-1H,5H,6H,7H-pyrrolo[3,2-c]pyridin-4-one (150 mg, 0.41 mmol, 1.00 equiv) and tert-butyl (2R)-2-ethynyl-4,4-difluoropyrrolidine-1-carboxylate (282 mg, 1.22 mmol, 3.00 equiv) in DMF (3.00 mL) were added Pd(dppf)Cl₂. DCM (148 mg, 0.20 mmol, 0.50 equiv), DIEA (157 mg, 1.22 mmol, 3.00 equiv) and CuI (38 mg, 0.20 mmol, 0.50 equiv) dropwise at room temperature. The resulting suspension was backfilled with argon three times and stirred for 2h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH2Cl2/MeOH (20:1) to afford tert-butyl (2R)-2-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]-4,4-difluoropyrrolidine-1-carboxylate (90 mg, 37.00%) as a yellow solid.

LC-MS: (M+H)⁺ found: 598.15.

### 121.4. Synthesis of 3-[(3-chloro-2-methoxyphenyl)amino]-2-(3-[2-[(2R)-4,4-difluoropyrrolidin-2-yl]ethynyl]pyridin-4-yl)-1H,5H,6H,7H-pyrrolo[3,2-c]pyridin-4-one

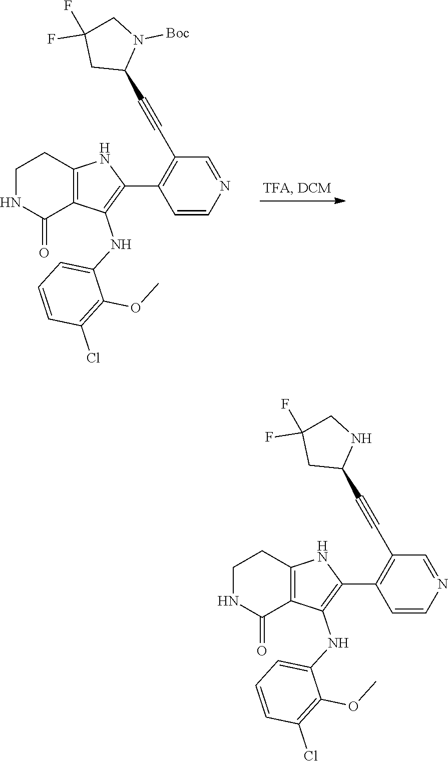 [SMILES: COc1c(Cl)cccc1Nc1c(-c2ccncc2C#C[C@H]2CC(F)(F)CN2C(=O)OC(C)(C)C)[nH]c2c1C(=O)NCC2 ; COc1c(Cl)cccc1Nc1c(-c2ccncc2C#C[C@H]2CC(F)(F)CN2)[nH]c2c1C(=O)NCC2]

To a stirred mixture of tert-butyl (2R)-2-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]-4,4-difluoropyrrolidine-1-carboxylate (200 mg, 0.33 mmol, 1.00 equiv) in DCM (3 mL) was added TFA (1 mL) at room temperature under nitrogen atmosphere. The resulting mixture was stirred for 1 h and concentrated under reduced pressure to afford 3-[(3-chloro-2-methoxyphenyl)amino]-2-(3-[2-[(2R)-4,4-difluoropyrrolidin-2-yl]ethynyl]pyridin-4-yl)-1H,5H,6H,7H-pyrrolo[3,2-c]pyridin-4-one (300 mg, crude) as a red oil.

LC-MS: (M+H)⁺ found: 498.05.

### 121.5. Synthesis of 3-[(3-chloro-2-methoxyphenyl)amino]-2-(3-[2-[(2R)-4,4-difluoro-1-(prop-2-enoyl)pyrrolidin-2-yl]ethynyl]pyridin-4-yl)-1H,5H,6H,7H-pyrrolo[3,2-c]pyridin-4-one

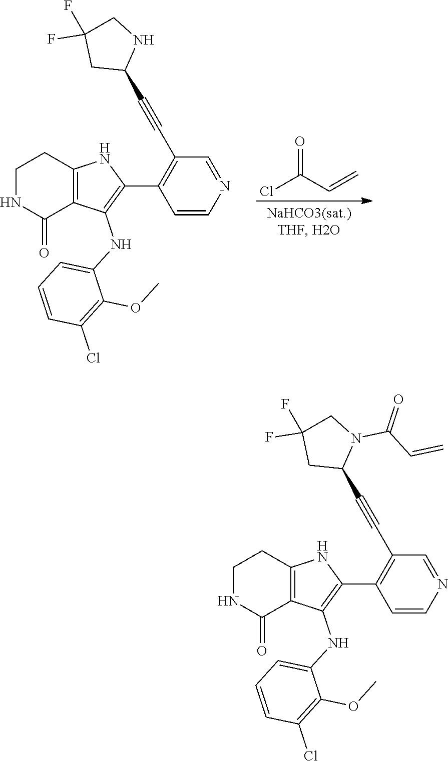

To a stirred mixture of 3-[(3-chloro-2-methoxyphenyl)amino]-2-(3-[2-[(2R)-4,4-difluoropyrrolidin-2-yl]ethynyl]pyridin-4-yl)-1H,5H,6H,7H-pyrrolo[3,2-c]pyridin-4-one (167 mg, 0.34 mmol, 1.00 equiv) in THF (2.00 mL) and NaHCO₃(2 mL) was added 5 acryloyl chloride (28 mg, 0.32 mmol, 0.95 equiv) dropwise at 0° C. The resulting mixture was stirred for 2 h at room temperature under nitrogen atmosphere. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by Prep-TLC (CH₂Cl₂/MeOH=20:1) to afford 3-[(3-chloro-2-methoxyphenyl)amino]-2-(3-[2-[(2R)-4,4-difluoro-1-(prop-2-enoyl)pyrrolidin-2-yl]ethynyl]pyridin-4-yl)-1H,5H,6H,7H-pyrrolo[3,2-c]pyridin-4-one (37.8 mg, 19.99%) as a yellow solid.

LC-MS: (M+H)⁺ found 552.00.

¹H-NMR (400 MHz, DMSO-d6) δ 11.12 (s, 1H), 8.50 (s, 1H), 8.28 (d, 1H), 7.30 (d, 2H), 6.84 (s, 1H), 6.58 (d, 3H), 6.29-5.97 (m, 2H), 5.70 (d, 1H), 5.27 (s, 1H), 4.03 (m, 2H), 3.85 (s, 3H), 3.53-3.35 (m, 2H), 2.88 (s, 2H), 2.78-2.56 (m, 2H).

### Example 122. 3-[(3-chloro-2-methoxyphenyl)amino]-2-(3-[2-[1-(trifluoromethyl)cyclopropyl]ethynyl]pyridin-4-yl)-1H,5H,6H,7H-pyrrolo[3,2-c]pyridin-4-one (compound 528)

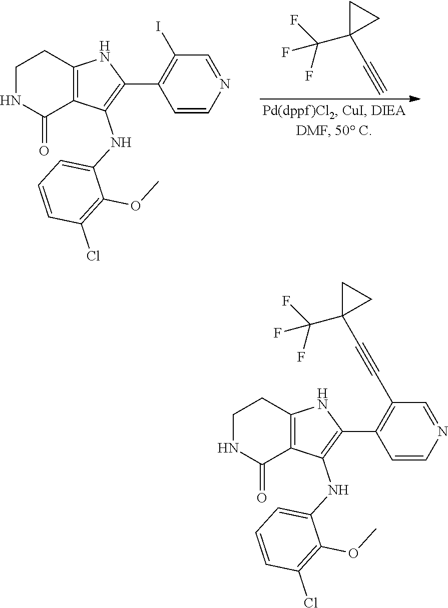

To a stirred mixture of 3-[(3-chloro-2-methoxyphenyl)amino]-2-(3-iodopyridin-4-yl)-1H,5H,6H,7H-pyrrolo[3,2-c]pyridin-4-one (100 mg, 0.20 mmol, 1.00 equiv) in DMF (2 mL) were added Pd(dppf)Cl₂CH₂Cl₂ (82 mg, 0.10 mmol, 0.50 equiv), CuI (19 mg, 0.10 mmol, 0.50 equiv), DIEA (78 mg, 0.60 mmol, 3.00 equiv) and 1-ethynyl-1-(trifluoromethyl)cyclopropane (108 mg, 0.81 mmol, 4.00 equiv) in portions at room temperature. The resulting suspension was backfilled with argon three times and stirred for 2h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to obtain crude product. The crude product (90 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 58% B in 7 min; Wave Length: 254 nm; RT1(min): 6.5; Number Of Runs: 0) to afford 3-[(3-chloro-2-methoxyphenyl)amino]-2-(3-[2-[1-(trifluoromethyl)cyclopropyl]ethynyl]pyridin-4-yl)-1H,5H,6H,7H-pyrrolo[3,2-c]pyridin-4-one (47.2 mg, 45.22%) as a yellow solid.

LC-MS: (M+H)⁺ found 501.00.

1H NMR (400 MHz, Methanol-d4) δ 8.54 (s, 1H), 8.28 (s, 1H), 7.39 (s, 1H), 6.63-6.54 (m, 2H), 6.19-6.13 (m, 1H), 3.91 (s, 3H), 3.58 (t, 2H), 2.93 (t, 2H), 1.50-1.35 (m, 4H).

### Example 123. 3-[(3-chloro-2-methoxyphenyl)amino]-2-(3-[2-[2-(prop-2-enoyl)-2-azabicyclo[3.1.0]hexan-3-yl]ethynyl]pyridin-4-yl)-1H,5H,6H,7H-pyrrolo[3,2-c]pyridin-4-one (compound 560)

### 123.1. Synthesis of tert-butyl 3-(methoxy(methyl)carbamoyl)-2-azabicyclo[3.1.0]hexane-2-carboxylate

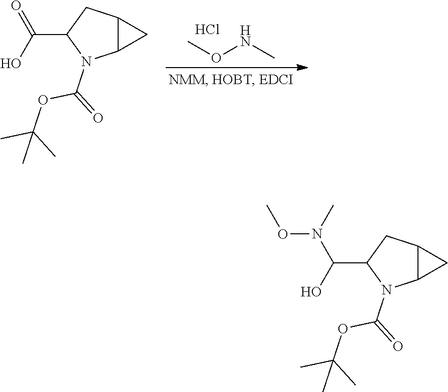

To a stirred solution of 2-(tert-butoxycarbonyl)-2-azabicyclo[3.1.0]hexane-3-carboxylic acid (2.5 g, 11.01 mmol, 1.00 equiv) in dry DMF at 0° C., N,O-Dimethylhydroxylamine hydrochloride (1.3 g, 13.21 mmol, 1.20 equiv) was added. After stirring for 15 minutes at 0° C., NMM (1.33 g, 13.21 mmol, 1.20 equiv), HOBT (1.78 g, 13.21 mmol, 1.20 equiv) and EDCI (2.55 g, 13.21 mmol, 1.20 equiv) was added at the same temperature. The reaction mixture was stirred for overnight at room temperature and subjected to dilute aqueous HCl workup. The resulting mixture was concentrated under vacuum. Flash chromatography on a short pad silica gel using 1:1 PE/EA as the eluent afforded tert-butyl 3-(methoxy(methyl)carbamoyl)-2-azabicyclo[3.1.0]hexane-2-carboxylate (2.9 g, 97%) as white solid.

LC-MS: (M+H−56)⁺ found 215.

### 123.2. Synthesis of tert-butyl 3-ethynyl-2-azabicyclo[3.1.0]hexane-2-carboxylate

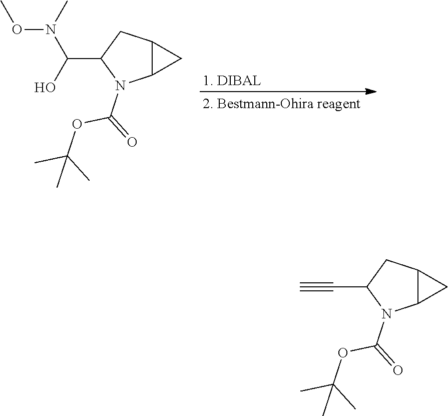 [SMILES: CON(C)C(O)C1CC2CC2N1C(=O)OC(C)(C)C ; C#CC1CC2CC2N1C(=O)OC(C)(C)C]

To a solution of tert-butyl 3-(methoxy(methyl)carbamoyl)-2-azabicyclo[3.1.0]hexane-2-carboxylate (2.6 g, 9.63 mmol, 1.00 equiv) in dry dichloromethane (20 mL) at −78° C. under argon atmosphere, DIBAL-H (12 mL, 11.56 mmol, 1.20 equiv, 1.0 M in dichloromethane) was added dropwise. The reaction was stirred at this temperature for another 2 h (TLC monitors the completion), the residue DIBAL-H was quenched by dropwise addition of anhydrous MeOH. The resulting mixture was filtered, the filter cake was washed with MeOH. The filtrate was concentrated under reduced pressure. The reaction was then allowed to warm to 0° C., K₂CO₃ (2.66 g, 19.26 mmol, 2.00 equiv), Bestmann-Ohira reagent (2.22 g, 11.56 mmol, 1.20 equiv) and anhydrous MeOH (30 mL) was added. The reaction mixture was stirred overnight at room temperature, then saturated seignette salt (30 mL) and diethyl ether (50 mL) were added, and the mixture was vigorously stirred for 1 h. The organic layer was separated and extracted with diethyl ether (3×40 mL), washed with brine, and dried over Na₂SO₄. The solvents were removed in a rotary evaporator. The residue was purified with flash chromatography on a short pad silica gel using 10:1 PE/EA as the eluent to give the tert-butyl 3-ethynyl-2-azabicyclo[3.1.0]hexane-2-carboxylate (1.5 g, 80%) as white solid.

¹H NMR (300 MHz, Chloroform-d) δ 4.31 (t, 1H), 3.34 (s, 1H), 2.50-2.21 (m, 3H), 1.73-1.57 (m, 1H), 1.49 (s, 9H), 0.89-0.81 (m, 1H), 0.41 (s, 1H).

### 123.3. Synthesis of tert-butyl 3-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]-2-azabicyclo[3.1.0]hexane-2-carboxylate

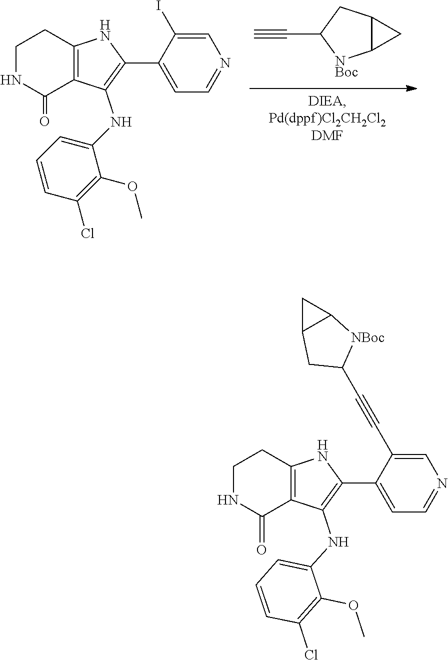

A solution of 3-[(3-chloro-2-methoxyphenyl)amino]-2-(3-iodopyridin-4-yl)-1H,5H,6H,7H-pyrrolo[3,2-c]pyridin-4-one (200 mg, 0.40 mmol, 1.00 equiv) and tert-butyl 3-ethynyl-2-azabicyclo[3.1.0]hexane-2-carboxylate (335 mg, 1.61 mmol, 4.00 equiv), and CuI (38 mg, 0.20 mmol, 0.50 equiv) and DIEA (781 mg, 6.06 mmol, 15 equiv) and Pd(dppf)Cl₂CH₂Cl₂ (33 mg, 0.04 mmol, 0.10 equiv) in DMF (5.00 mL) was stirred for 72 h at 50 degrees C. under argon atmosphere.

The mixture was allowed to cool down to room temperature. The reaction was quenched by the addition of Water (5 mL) at room temperature. The resulting mixture was extracted with EtOAc (2×30 mL). The combined organic layers were washed with brine (2×20 mL), dried over anhydrous Na₂SO₄. After filtration, the filtrate was concentrated under reduced pressure. The residue was purified by reverse flash chromatography with the following conditions: column, C18 silica gel; mobile phase, MeCN in water (NH₄HCO₃), 20% to 80% gradient in 30 min; detector, UV 254 nm. This resulted in tert-butyl 3-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]-2-azabicyclo[3.1.0]hexane-2-carboxylate (180 mg, 77.6%) as a yellow solid.

LC-MS: (M+H)⁺ found: 574.10

### 123.4. Synthesis of 2-[3-(2-[2-azabicyclo[3.1.0]hexan-3-yl]ethynyl)pyridin-4-yl]-3-[(3-chloro-2-methoxyphenyl)amino]-1H,5H,6H,7H-pyrrolo[3,2-c]pyridin-4-one

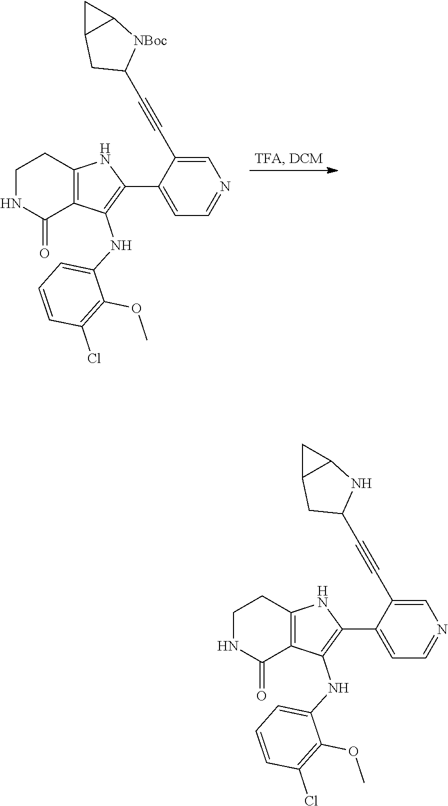 [SMILES: COc1c(Cl)cccc1Nc1c(-c2ccncc2C#CC2CC3CC3N2C(=O)OC(C)(C)C)[nH]c2c1C(=O)NCC2 ; COc1c(Cl)cccc1Nc1c(-c2ccncc2C#CC2CC3CC3N2)[nH]c2c1C(=O)NCC2]

A solution of tert-butyl 3-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]-2-azabicyclo[3.1.0]hexane-2-carboxylate (160 mg, 0.28 mmol, 1.00 equiv) and TFA (5.00 mL) in DCM (5.00 ml) was stirred for overnight at room temperature under nitrogen atmosphere. The resulting mixture was concentrated under reduced pressure. This resulted in 2-[3-(2-[2-azabicyclo[3.1.0]hexan-3-yl]ethynyl)pyridin-4-yl]-3-[(3-chloro-2-methoxyphenyl)amino]-1H,5H,6H,7H-pyrrolo[3,2-c]pyridin-4-one (140 mg, crude) as a brown solid.

LC-MS: (M+H)⁺ found: 474.20

### 123.5. Synthesis of 3-[(3-chloro-2-methoxyphenyl)amino]-2-(3-[2-[2-(prop-2-enoyl)-2-azabicyclo[3.1.0]hexan-3-yl]ethynyl]pyridin-4-yl)-1H,5H,6H,7H-pyrrolo[3,2-c]pyridin-4-one

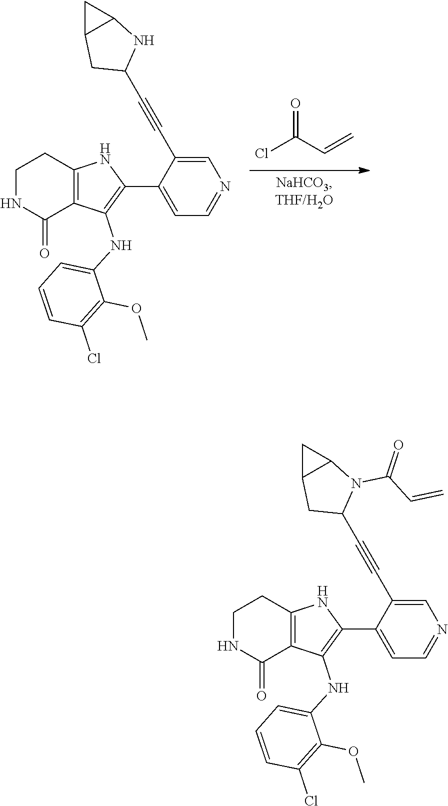

A solution of 2-[3-(2-[2-azabicyclo[3.1.0]hexan-3-yl]ethynyl)pyridin-4-yl]-3-[(3-chloro-2-methoxyphenyl)amino]-1H,5H,6H,7H-pyrrolo[3,2-c]pyridin-4-one (140 mg, 0.29 mmol, 1.00 equiv) in THF (1.00 mL) and sat. NaHCO₃(aq.) (1.00 mL) was stirred for 30 min at 0° C. under nitrogen atmosphere. To the above mixture was added acryloyl chloride (27 mg, 0.29 mmol, 1.00 equiv) dropwise over 1 min at 0° C. The resulting mixture was stirred for additional 2h at room temperature. The reaction was quenched with water/ice at 0° C. The resulting mixture was extracted with EtOAc (2×50 mL). The combined organic layers were washed with brine (1×120 mL), dried over anhydrous Na₂SO₄. After filtration, the filtrate was concentrated under reduced pressure. The residue was purified by reverse flash chromatography with the following conditions: Column: XBridge Prep C18 OBD Column, 30*100 mm, 5¦Ìm; Mobile Phase A: Water (10 mmol/L NH₄HCO₃+0.1% NH₃·H₂O), Mobile Phase B: ACN; Flow rate: 60 mL/min; Gradient: 23% B to 53% B in 10 min, 53% B; Wave Length: 254/220 nm; RT1(min): 7.53; Number Of Runs: 0. This resulted in 3-[(3-chloro-2-methoxyphenyl)amino]-2-(3-[2-[2-(prop-2-enoyl)-2-azabicyclo[3.1.0]hexan-3-yl]ethynyl]pyridin-4-yl)-1H,5H,6H,7H-pyrrolo[3,2-c]pyridin-4-one (12.3 mg, 7.93%) as a yellow solid.

LC-MS: (M+H)⁺ found 528.00.

¹H NMR (300 MHz, DMSO-d6) δ 11.36 (s, 1H), 8.55 (s, 1H), 8.28 (d, 1H), 7.55 (s, 1H), 7.35 (d, 1H), 7.19 (s, 1H), 6.95-6.84 (m, 1H), 6.67 (d, 2H), 6.33-6.23 (m, 1H), 6.12 (t, 1H), 5.86-5.75 (m, 1H), 4.89 (t, 1H), 3.86 (s, 3H), 3.73 (s, 1H), 3.49-3.37 (m, 2H), 2.98 (t, 2H), 2.41-2.33 (m, 2H), 1.95-1.85 (m, 1H), 1.01-0.91 (m, 1H), 0.69-0.62 (s, 1H).

### Example 124. N-{1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]cyclopropyl}prop-2-enamide (compound 207)

### 124.1. Synthesis of N-{1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]cyclopropyl}carbamate

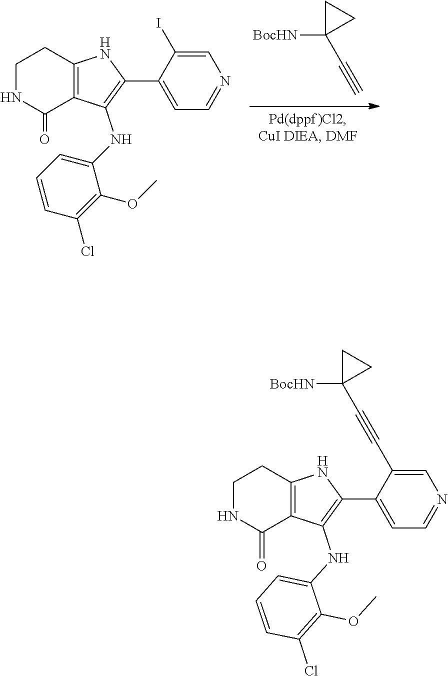 [SMILES: COc1c(Cl)cccc1Nc1c(-c2ccncc2I)[nH]c2c1C(=O)NCC2 ; C#CC1(NC(=O)OC(C)(C)C)CC1 ; COc1c(Cl)cccc1Nc1c(-c2ccncc2C#CC2(NC(=O)OC(C)(C)C)CC2)[nH]c2c1C(=O)NCC2]

To a solution of 3-[(3-chloro-2-methoxyphenyl)amino]-2-(3-iodopyridin-4-yl)-1H,5H,6H,7H-pyrrolo[3,2-c]pyridin-4-one (100 mg, 0.20 mmol, 1.00 equiv) and tert-butyl N-(1-ethynylcyclopropyl) Carbamate (73 mg, 0.40 mmol, 2.00 equiv) in DMF (2.00 mL) and DIEA (78 mg, 0.61 mmol, 3.00 equiv) were added CuI (19 mg, 0.10 mmol, 0.50 equiv) and Pd(dppf)Cl₂CH₂Cl₂ (8 mg, 0.01 mmol, 0.25 equiv). The resulting suspension was backfilled with argon three times and stirred for 2h at 50° C. LCMS confirmed completion of reaction and desired product was observed. The resulting mixture was concentrated under vacuum. The residue was purified by silica gel column chromatography, eluted with MeOH/DCM (3%) to afford tert-butyl N-{1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]cyclopropyl}carbamate (60 mg, 51.45%) as a yellow solid.

LC-MS: (M+H)⁺ found: 548.10.

### 124.2. Synthesis of 2-(3-((1-aminocyclopropyl)ethynyl)pyridin-4-yl)-3-((3-chloro-2-methoxyphenyl)amino)-1,5,6,7-tetrahydro-4H-pyrrolo[3,2-c]pyridin-4-one

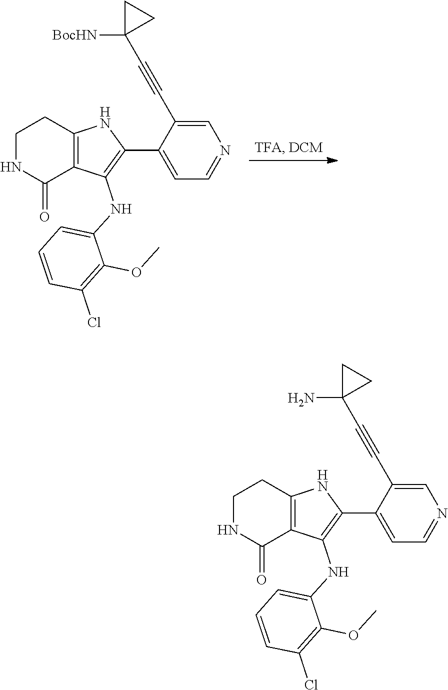 [SMILES: COc1c(Cl)cccc1Nc1c(-c2ccncc2C#CC2(NC(=O)OC(C)(C)C)CC2)[nH]c2c1C(=O)NCC2 ; COc1c(Cl)cccc1Nc1c(-c2ccncc2C#CC2(N)CC2)[nH]c2c1C(=O)NCC2]

Into a tert-butyl N-{1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]cyclopropyl}carbamate (50 mg, 0.09 mmol, 1.00 equiv) were added TFA (0.30 mL) and DCM (0.60 mL) at 0° C. The resulting mixture was stirred for 1 h at room temperature. The reaction was monitored by LCMS. The resulting mixture was stirred for 1 h and concentrated under reduced pressure to afford 2-(3-((1-aminocyclopropyl)ethynyl)pyridin-4-yl)-3-((3-chloro-2-methoxyphenyl)amino)-1,5,6,7-tetrahydro-4H-pyrrolo[3,2-c]pyridin-4-one (80 mg, crude) as a red oil.

LC-MS: (M+H)⁺ found: 448.05.

### 124.3. Synthesis of N-{1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]cyclopropyl}prop-2-enamide

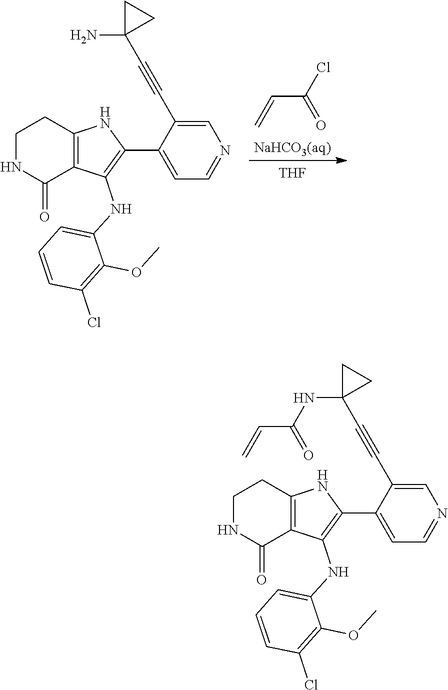

To a stirred solution of 2-{3-[2-(1-aminocyclopropyl)ethynyl]pyridin-4-yl}-3-[(3-chloro-2-methoxyphenyl) amino]-1H,5H,6H,7H-pyrrolo[3,2-c]pyridin-4-one (50 mg, 0.11 mmol, 1.00 equiv) and sat. NaHCO₃(aq.) (0.60 mL) in tetrahydrofuran (0.60 mL) was added acryloyl chloride (10 mg, 0.11 mmol, 1.00 equiv) dropwise at 0° C. under argon atmosphere. The resulting mixture was stirred for 1 h at room temperature under argon atmosphere. The reaction was monitored by LCMS. The resulting mixture was extracted with CH₂Cl₂/MeOH (10/1) (3×20 mL). The combined organic layers were washed with brine (3×20 mL), dried over anhydrous Na₂SO₄. After filtration, the filtrate was concentrated under reduced pressure. The crude product (80 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 22% B to 47% B in 9 min, 47% B; Wave Length: 254/220 nm; RT1(min): 8.85; Number Of Runs: 0) to afford N-{1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]cyclopropyl}prop-2-enamide (9.5 mg, 16.9%) as a yellow solid.

LC-MS: (M+H)⁺ found: 502.30

¹H NMR (300 MHz, DMSO-d6) δ 11.37 (s, 1H), 8.94 (s, 1H), 8.50 (s, 1H), 8.26 (d, 1H), 7.51 (s, 1H), 7.31 (d, 1H), 7.16 (s, 1H), 6.66 (d, 2H), 6.17 (d, 2H), 6.10 (t, 1H), 5.66 (t, 1H), 3.86 (s, 3H), 3.47-3.42 (m, 2H), 2.97 (t, 2H), 1.35-1.31 (m, 2H), 1.17-1.13 (m, 2H).

### Example 125. N-{1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]cyclopropyl}prop-2-enamide (compound 206)

### 125.1. Synthesis of tert-butyl N-{1-[2-(4-{3-[(3-fluoro-2-methoxyphenyl)amino]-4-oxo-1H,5H,6H,7H-pyrrolo[3,2-c]pyridin-2-yl}pyridin-3-yl) ethynyl]cyclopropyl}carbamate

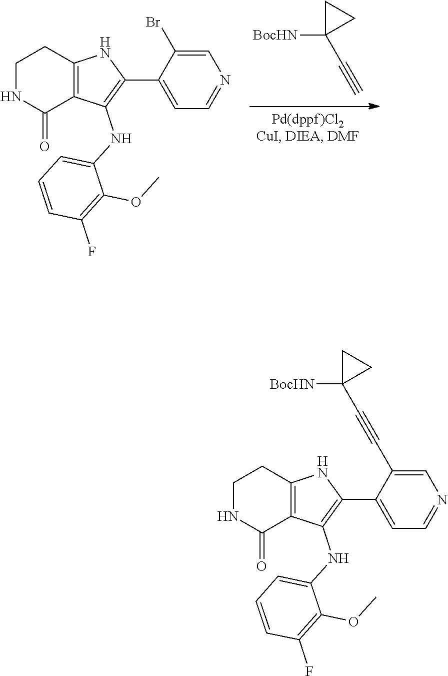 [SMILES: COc1c(F)cccc1Nc1c(-c2ccncc2Br)[nH]c2c1C(=O)NCC2 ; C#CC1(NC(=O)OC(C)(C)C)CC1 ; COc1c(F)cccc1Nc1c(-c2ccncc2C#CC2(NC(=O)OC(C)(C)C)CC2)[nH]c2c1C(=O)NCC2]

To a stirred solution of 2-(3-bromopyridin-4-yl)-3-[(3-fluoro-2-methoxyphenyl)amino]-1H,5H,6H,7H-pyrrolo[3,2-c]pyridin-4-one (800 mg, 1.86 mmol, 1.00 equiv) and tert-butyl N-(1-ethynylcyclopropyl) carbamate (1.01 g, 5.57 mmol, 3.00 equiv) in DMF (15 mL) were added DIEA (599 mg, 4.64 mmol, 2.50 equiv), CuI (177 mg, 0.93 mmol, 0.50 equiv) and Pd(dppf)Cl₂CH₂Cl₂ (378 mg, 0.46 mmol, 0.25 equiv) dropwise at room temperature under argon atmosphere. The resulting suspension was backfilled with argon three times and stirred for 2h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by reverse flash chromatography with the following conditions: column, C18 silica gel; mobile phase, ACN in water, 0% to 100% gradient in 10 min; detector, UV 254 nm to afford crude product. The residue was purified by silica gel column chromatography, eluted with MeOH/DCM (3%) to afford tert-butyl N-{1-[2-(4-{3-[(3-fluoro-2-methoxyphenyl)amino]-4-oxo-1H,5H,6H,7H-pyrrolo[3,2-c]pyridin-2-yl}pyridin-3-yl) ethynyl]cyclopropyl}carbamate (500 mg, 48.17%) as a yellow solid.

LC-MS: (M+H)⁺ found: 532.10.

### 125.2. Synthesis of 2-{3-[2-(1-aminocyclopropyl)ethynyl]pyridin-4-yl}-3-[(3-fluoro-2-methoxyphenyl) amino]-1H,5H,6H,7H-pyrrolo[3,2-c]pyridin-4-one

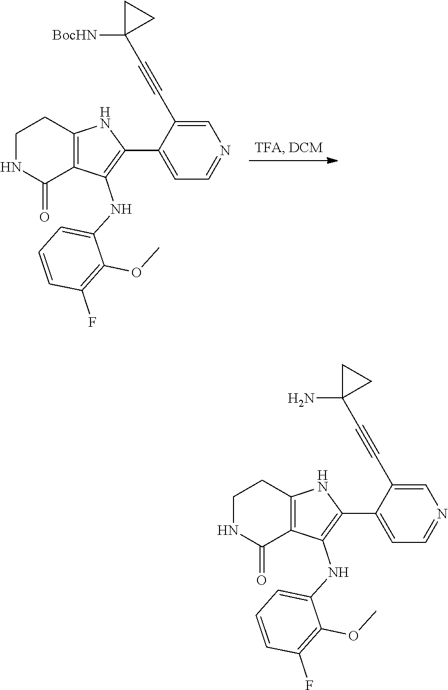 [SMILES: COc1c(F)cccc1Nc1c(-c2ccncc2C#CC2(NC(=O)OC(C)(C)C)CC2)[nH]c2c1C(=O)NCC2 ; COc1c(F)cccc1Nc1c(-c2ccncc2C#CC2(N)CC2)[nH]c2c1C(=O)NCC2]

Into a DCM (4.50 mL) were added tert-butyl N-{1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]cyclopropyl}carbamate (200 mg, 0.38 mmol, 1.00 equiv) and TFA (1.50 mL) at 0° C. The resulting mixture was stirred for 1 h and concentrated under reduced pressure. The residue was purified by reverse flash chromatography with the following conditions: column, silica gel; mobile phase, MeCN in water, 0% to 100% gradient in 10 min; detector, UV 254 nm to afford 2-{3-[2-(1-aminocyclopropyl)ethynyl]pyridin-4-yl}-3-[(3-fluoro-2-methoxyphenyl) amino]-1H,5H,6H,7H-pyrrolo[3,2-c]pyridin-4-one (120 mg, 70.23%) as a yellow solid.

LC-MS: (M+H)⁺ found: 432.05.

### 125.3. Synthesis of N-{1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]cyclopropyl}prop-2-enamide

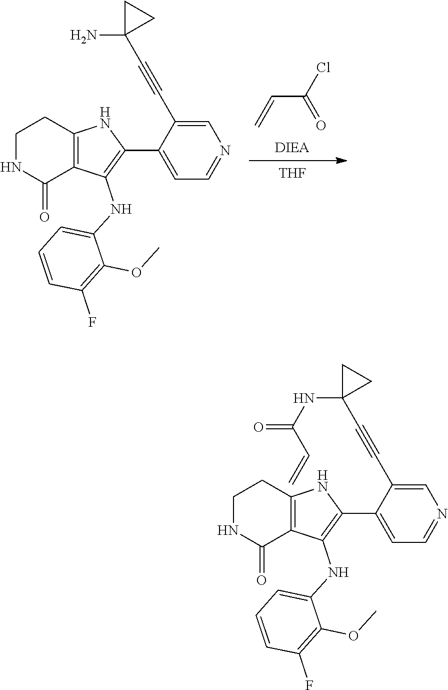

To a stirred solution 2-{3-[2-(1-aminocyclopropyl)ethynyl]pyridin-4-yl}-3-[(3-fluoro-2-methoxyphenyl) amino]-1H,5H,6H,7H-pyrrolo[3,2-c]pyridin-4-one (200 mg, 0.46 mmol, 1.00 equiv) and DIEA (180 mg, 1.39 mmol, 3.00 equiv) in tetrahydrofuran (2.00 mL) was added acryloyl chloride (38 mg, 0.04 mmol, 0.90 equiv) dropwise at 0° C. The resulting mixture was stirred for 1 h and concentrated under reduced pressure. The residue was purified by silica gel column chromatography, eluted with MeOH/DCM (3%) to afford N-{1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]cyclopropyl}prop-2-enamide (160 mg crude). The crude product (160 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 20% B to 45% B in 9 min, 45% B; Wave Length: 254/220 nm; RT1(min): 8.65; Number Of Runs: 0) to afford N-{1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]cyclopropyl}prop-2-enamide (100 mg, 44.39%) as an off-white solid.

LC-MS: (M+H)⁺ found: 486.10.

¹H NMR (400 MHz, DMSO-d6) δ 11.36 (s, 1H), 8.97 (s, 1H), 8.50 (s, 1H), 8.26 (d, 1H), 7.53 (s, 1H), 7.32 (d, 1H), 7.19 (s, 1H), 6.64-6.61 (m, 1H), 6.50-6.45 (m, 1H), 6.18 (d, 2H), 5.95 (d, 1H), 5.67 (t, 1H), 3.90 (s, 3H), 3.45-3.42 (m, 2H), 2.97 (t, 2H), 1.35-1.32 (m, 2H), 1.17-1.14 (m, 2H).

### Example 126. 3-[(3-fluoro-2-methoxyphenyl)amino]-2-(3-{2-[(1S,5R)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one (compound 205)

### 126.1. Synthesis of tert-butyl 1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate

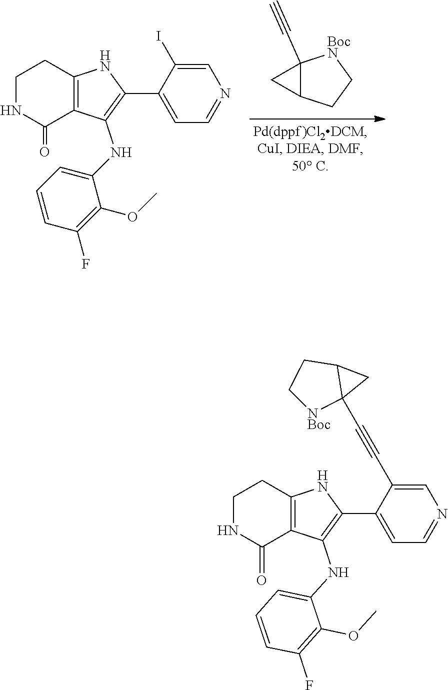 [SMILES: COc1c(F)cccc1Nc1c(-c2ccncc2I)[nH]c2c1C(=O)NCC2 ; C#CC12CC1CCN2C(=O)OC(C)(C)C ; COc1c(F)cccc1Nc1c(-c2ccncc2C#CC23CC2CCN3C(=O)OC(C)(C)C)[nH]c2c1C(=O)NCC2]

To a stirred mixture of 3-[(3-fluoro-2-methoxyphenyl)amino]-2-(3-iodopyridin-4-yl)-1H,5H,6H,7H-pyrrolo[3,2-c]pyridin-4-one (200 mg, 0.42 mmol, 1.00 equiv), CuI (20 mg, 0.10 mmol, 0.50 equiv) and Pd(dppf)Cl₂·CH₂Cl₂ (170 mg, 0.21 mmol, 0.50 equiv) in DMF (2.00 mL) were added tert-butyl 1-ethynyl-2-azabicyclo[3.1.0]hexane-2-carboxylate (260 mg, 1.25 mmol, 3.00 equiv) and DIEA (270 mg, 2.09 mmol, 5.00 equiv) dropwise at room temperature. The final reaction mixture was stirred for 2h at 50 degrees C. under argon atmosphere. The mixture was concentrated under reduced pressure. The residue was purified by reverse flash chromatography with the following conditions: column, C18 gel; mobile phase, ACN in water, 10% to 60% gradient in 25 min; detector, UV 254 nm. This resulted in tert-butyl 1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (250 mg, 76.12%) as a yellow solid.

LC-MS: (M+H)⁺ found: 558.15.

### 126.2. Synthesis of 2-(3-((2-azabicyclo[3.1.0]hexan-1-yl)ethynyl)pyridin-4-yl)-3-((3-fluoro-2-methoxyphenyl)amino)-1,5,6,7-tetrahydro-4H-pyrrolo[3,2-c]pyridin-4-one

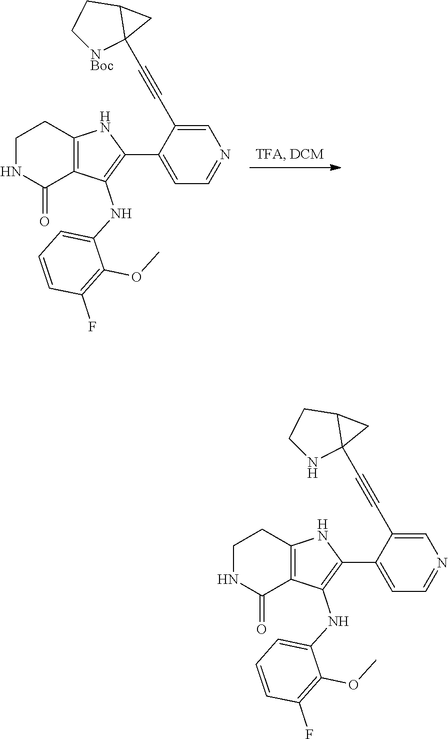 [SMILES: COc1c(F)cccc1Nc1c(-c2ccncc2C#CC23CC2CCN3C(=O)OC(C)(C)C)[nH]c2c1C(=O)NCC2 ; COc1c(F)cccc1Nc1c(-c2ccncc2C#CC23CC2CCN3)[nH]c2c1C(=O)NCC2]

To a stirred solution of tert-butyl 1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (220 mg, 0.39 mmol, 1.00 equiv) in DCM (6.00 mL) was added TFA (2.00 mL) dropwise at room temperature under nitrogen atmosphere. The resulting mixture was stirred for 1 h at room temperature under nitrogen atmosphere. The resulting mixture was concentrated under reduced pressure. The crude product was used in the next step directly without further purification.

LC-MS: (M+H)⁺ found: 458.1.

### 126.3. Synthesis of 3-[(3-fluoro-2-methoxyphenyl)amino]-2-(3-{2-[2-(prop-2-enoyl)-2-azabicyclo[3.1.0]hexan-1-yl]ethynyl}pyridin-4-yl)-1H,5H,6H,7H-pyrrolo[3,2-c]pyridin-4-one

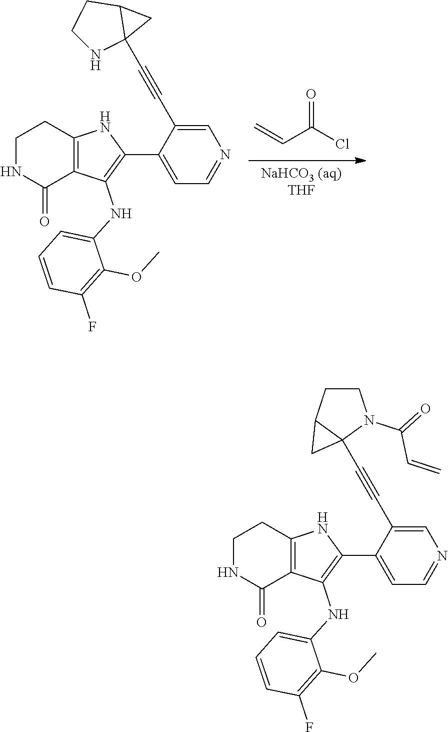

To a stirred solution of 2-[3-(2-{2-azabicyclo[3.1.0]hexan-1-yl}ethynyl)pyridin-4-yl]-3-[(3-fluoro-2-methoxyphenyl)amino]-1H,5H,6H,7H-pyrrolo[3,2-c]pyridin-4-one (180 mg, 0.39 mmol, 1.00 equiv) in THF (6.00 mL) and NaHCO₃(sat.) (6.00 mL) was added acryloyl chloride (32 mg, 0.35 mmol, 0.9 equiv) dropwise at 0 degrees C. under nitrogen atmosphere. The resulting mixture was stirred for 1 h at room temperature under nitrogen atmosphere. The resulting mixture was concentrated under reduced pressure. The residue was purified by silica gel column chromatography, eluted with DCM/MeOH (10:1) to afford 3-[(3-fluoro-2-methoxyphenyl)amino]-2-(3-{2-[2-(prop-2-enoyl)-2-azabicyclo[3.1.0]hexan-1-yl]ethynyl}pyridin-4-yl)-1H,5H,6H,7H-pyrrolo[3,2-c]pyridin-4-one (30 mg, 14.86%) as a yellow solid.

LC-MS: (M+H)⁺ found: 512.1.

### 126.4. Synthesis of 3-[(3-fluoro-2-methoxyphenyl)amino]-2-(3-{2-[(1S,5R)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one

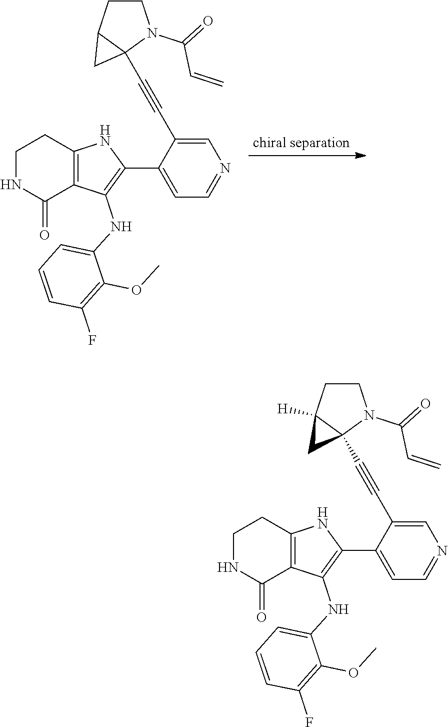 [SMILES: C=CC(=O)N1CCC2CC21C#Cc1cnccc1-c1[nH]c2c(c1Nc1cccc(F)c1OC)C(=O)NCC2 ; C=CC(=O)N1CC[C@@H]2C[C@@]21C#Cc1cnccc1-c1[nH]c2c(c1Nc1cccc(F)c1OC)C(=O)NCC2]

The crude product (30 mg) was purified by Prep-CHIRAL-HPLC with the following conditions (Column: CHIRALPAK IG, 2*25 cm, 5 μm; Mobile Phase A: Hex:DCM=3:1(0.5% 2M NH₃-MeOH)—HPLC, Mobile Phase B: EtOH—HPLC; Flow rate: 20 mL/min; Gradient: 20% B to 20% B in 20 min; Wave Length: 220/254 nm; RT1(min): 10.98; RT2(min): 17.32; Sample Solvent: ETOH:DCM=1:1; Injection Volume: 0.8 mL; Number Of Runs: 5) to afford 3-[(3-fluoro-2-methoxyphenyl)amino]-2-(3-{2-[(1S,5R)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one (8.8 mg, 29.16%) as a yellow solid.

LC-MS: (M+H)⁺ found: 512.00.

¹H NMR (400 MHz, DMSO-d6) δ 11.55 (s, 1H), 8.43 (s, 1H), 8.27 (d, 1H), 7.40 (d, 1H), 6.91 (s, 1H), 6.71-6.59 (m, 4H), 6.47 (t, 1H), 6.16-6.10 (m, 1H), 5.66-5.39 (m, 1H), 4.09-3.93 (m, 2H), 3.46-3.42 (m, 2H), 3.31 (s, 1H), 2.92-2.87 (m, 3H), 2.40-2.28 (m, 1H), 2.20-2.10 (m, 1H), 1.78-1.64 (m, 1H), 0.92 (s, 1H).

### Example 127. 3-[(3-fluoro-2-methoxyphenyl)amino]-2-(3-{2-[(1S,5R)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one (compound 204)

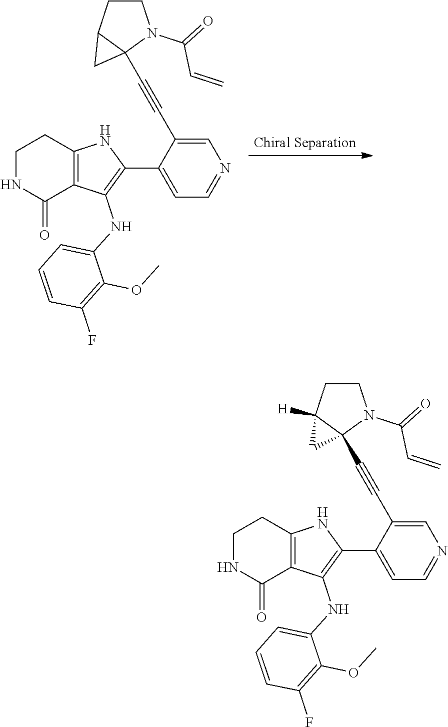 [SMILES: C=CC(=O)N1CCC2CC21C#Cc1cnccc1-c1[nH]c2c(c1Nc1cccc(F)c1OC)C(=O)NCC2 ; C=CC(=O)N1CC[C@H]2C[C@]21C#Cc1cnccc1-c1[nH]c2c(c1Nc1cccc(F)c1OC)C(=O)NCC2]

The crude product (30 mg) was purified by Prep-CHIRAL-HPLC with the following conditions (Column: CHIRALPAK IG, 2*25 cm, 5 μm; Mobile Phase A: Hex:DCM=3:1(0.5% 2M NH₃-MeOH)-HPLC, Mobile Phase B: EtOH—HPLC; Flow rate: 20 mL/min; Gradient: 20% B to 20% B in 20 min; Wave Length: 220/254 nm; RT1(min): 10.98; RT2(min): 17.32; Sample Solvent: ETOH:DCM=1:1; Injection Volume: 0.8 mL; Number Of Runs: 5) to afford 3-[(3-fluoro-2-methoxyphenyl)amino]-2-(3-{2-[(1S,5R)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one (8.8 mg, 29.16%) as a yellow solid.

LC-MS: (M+H)⁺ found: 512.00

¹H NMR (400 MHz, DMSO-d6) δ 11.55 (s, 1H), 8.43 (s, 1H), 8.28 (d, 1H), 7.40 (d, 1H), 7.05-6.60 (m, 5H), 6.53-6.42 (m, 1H), 6.18-6.03 (m, 1H), 5.56 (d, 1H), 4.09-3.77 (m, 2H), 3.49-3.40 (m, 2H), 3.30 (s, 3H), 2.93-2.84 (m, 3H), 2.36-2.25 (m, 1H), 2.18-2.07 (m, 1H), 1.73-1.62 (m, 1H), 0.94 (s, 1H).

### Example 128. 3-[(3-chloro-2-methoxyphenyl)amino]-2-(3-{2-[(1R,5S)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one (compound 203)

### 128.1. Synthesis of tert-butyl 1-(hydroxymethyl)-2-azabicyclo[3.1.0]hexane-2-carboxylate

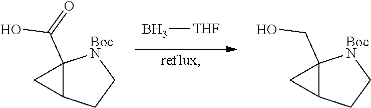

To a stirred solution of 2-(tert-butoxycarbonyl)-2-azabicyclo[3.1.0]hexane-1-carboxylic acid (1.5 g, 6.60 mmol, 1.00 equiv) in THF (30 mL) was added BH₃ (1 M in THF, 7.26 mL, 7.26 mmol, 1.10 equiv) dropwise at 0 degrees C. under nitrogen atmosphere. The resulting mixture was stirred for 1 h at 80° C. under nitrogen atmosphere. The reaction was quenched by the addition of MeOH (10 mL) at 0° C. The resulting mixture was concentrated under reduced pressure. The crude product was used in the next step directly without further purification.

LC-MS: (M+H−56)⁺ found: 157.85. 128.2. Synthesis of tert-butyl 1-formyl-2-azabicyclo[3.1.0]hexane-2-carboxylate

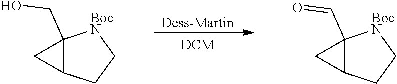

To a stirred solution of tert-butyl 1-(hydroxymethyl)-2-azabicyclo[3.1.0]hexane-2-carboxylate (1.71 g, 8.02 mmol, 1.00 equiv) in DCM (30 mL) was added Dess-Martin (4.08 g, 9.62 mmol, 1.20 equiv) dropwise at 0 degrees C. under nitrogen atmosphere. The resulting mixture was stirred for 2 h at room temperature under nitrogen atmosphere. The resulting mixture was concentrated under reduced pressure. The residue was purified by silica gel column chromatography, eluted with PE/EA (5:1) to afford tert-butyl 1-formyl-2-azabicyclo[3.1.0]hexane-2-carboxylate (1.38 g, 81.47%) as a yellow oil.

LC-MS: (M+H−56)⁺ found: 155.85.

### 128.3. Synthesis of tert-butyl 1-ethynyl-2-azabicyclo[3.1.0]hexane-2-carboxylate

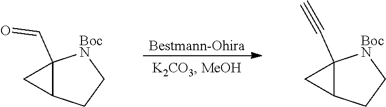

To a stirred solution of tert-butyl 1-formyl-2-azabicyclo[3.1.0]hexane-2-carboxylate (1.38 g, 6.53 mmol, 1.00 equiv) and K₂CO₃ (2.71 g, 19.59 mmol, 3.00 equiv) in MeOH (30 mL) was added dimethyl (1-diazo-2-oxopropyl)phosphonate (1.51 g, 7.84 mmol, 1.20 equiv) dropwise at 0 degrees C. under nitrogen atmosphere. The resulting mixture was stirred for 2 h at room temperature under nitrogen atmosphere. The reaction was quenched by the addition of potassium sodium tartrate (sat.) (10 mL) at room temperature. The mixture was stirred for 30 min. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (5:1) to afford tert-butyl 1-ethynyl-2-azabicyclo[3.1.0]hexane-2-carboxylate (1.18 g, 87.15%) as a yellow oil.

LC-MS: (M+H−56)⁺ found: 151.90.

### 128.4. Synthesis of tert-butyl 1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate

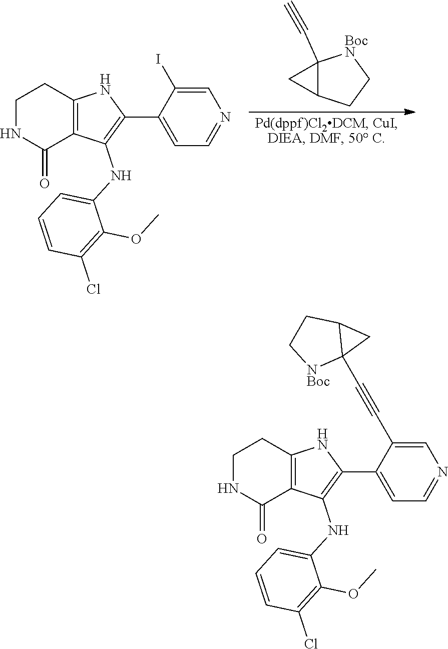

To a stirred solution of 3-[(3-chloro-2-methoxyphenyl)amino]-2-(3-iodopyridin-4-yl)-1H,5H,6H,7H-pyrrolo[3,2-c]pyridin-4-one (200 mg, 0.40 mmol, 1.00 equiv) and tert-butyl 1-ethynyl-2-azabicyclo[3.1.0]hexane-2-carboxylate (251 mg, 1.21 mmol, 3.00 equiv) in DMF (2.00 mL) were added Pd(dppf)Cl₂CH₂Cl₂ (82 mg, 0.10 mmol, 0.25 equiv) and DIEA (261 mg, 2.02 mmol, 5.00 equiv) dropwise at room temperature under argon atmosphere. The resulting mixture was stirred for 2 h at 50° C. under argon atmosphere. The mixture was concentrated under reduced pressure. The residue was purified by reverse flash chromatography with the following conditions: column, silica gel; mobile phase, MeCN in water, 10% to 70% gradient in 30 min; detector, UV 254 nm. This resulted in tert-butyl 1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (210 mg, 90.48%) as a yellow solid.

LC-MS: (M+H)⁺ found: 574.1.

### 128.5. Synthesis of 2-(3-((2-azabicyclo[3.1.0]hexan-1-yl)ethynyl)pyridin-4-yl)-3-((3-chloro-2-methoxyphenyl)amino)-1,5,6,7-tetrahydro-4H-pyrrolo[3,2-c]pyridin-4-one

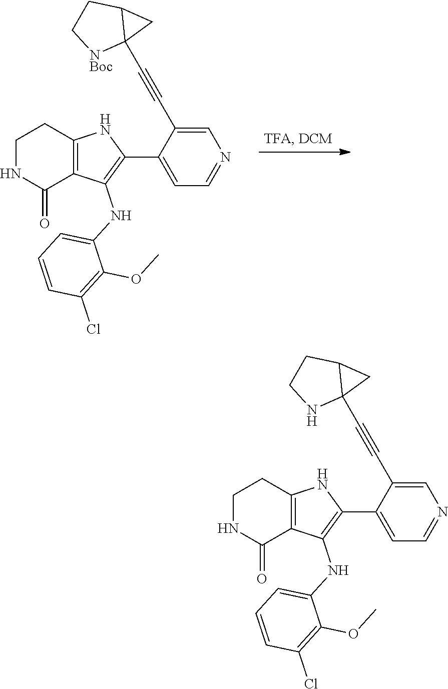 [SMILES: COc1c(Cl)cccc1Nc1c(-c2ccncc2C#CC23CC2CCN3C(=O)OC(C)(C)C)[nH]c2c1C(=O)NCC2 ; COc1c(Cl)cccc1Nc1c(-c2ccncc2C#CC23CC2CCN3)[nH]c2c1C(=O)NCC2]

To a stirred mixture of tert-butyl 1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (260 mg, 0.45 mmol, 1.00 equiv) in DCM (6.00 mL) was added TFA (2.00 mL) dropwise at room temperature under nitrogen atmosphere. The resulting mixture was stirred for 1 h at room temperature under nitrogen atmosphere. The resulting mixture was concentrated under reduced pressure. The crude product was used in the next step directly without further purification.

LC-MS: (M+H)⁺ found: 474.15.

### 128.6. Synthesis of 3-[(3-chloro-2-methoxyphenyl)amino]-2-(3-{2-[2-(prop-2-enoyl)-2-azabicyclo[3.1.0]hexan-1-yl]ethynyl}pyridin-4-yl)-1H,5H,6H,7H-pyrrolo[3,2-c]pyridin-4-one

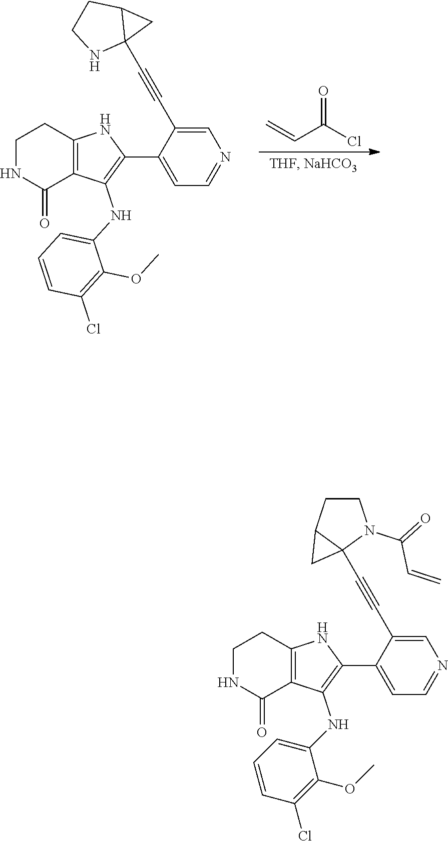

To a stirred solution of 2-[3-(2-{2-azabicyclo[3.1.0]hexan-1-yl}ethynyl)pyridin-4-yl]-3-[(3-chloro-2-methoxyphenyl)amino]-1H,5H,6H,7H-pyrrolo[3,2-c]pyridin-4-one (215 mg, 0.45 mmol, 1.00 equiv) and NaHCO₃(5.00 mL) in THF (5.00 mL) was added acryloyl chloride (36 mg, 0.41 mmol, 0.90 equiv) dropwise at 0 degrees C. under nitrogen atmosphere. The resulting mixture was concentrated under reduced pressure. The residue was purified by silica gel column chromatography, eluted with CH₂Cl₂/MeOH (15:1) to afford 3-[(3-chloro-2-methoxyphenyl)amino]-2-(3-{2-[2-(prop-2-enoyl)-2-azabicyclo[3.1.0]hexan-1-yl]ethynyl}pyridin-4-yl)-1H,5H,6H,7H-pyrrolo[3,2-c]pyridin-4-one (50 mg, 20.88%) as a yellow solid.

LC-MS: (M+H)⁺ found: 528.20.

### 128.7. Synthesis of 3-[(3-chloro-2-methoxyphenyl)amino]-2-(3-{2-[(1R,5S)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one

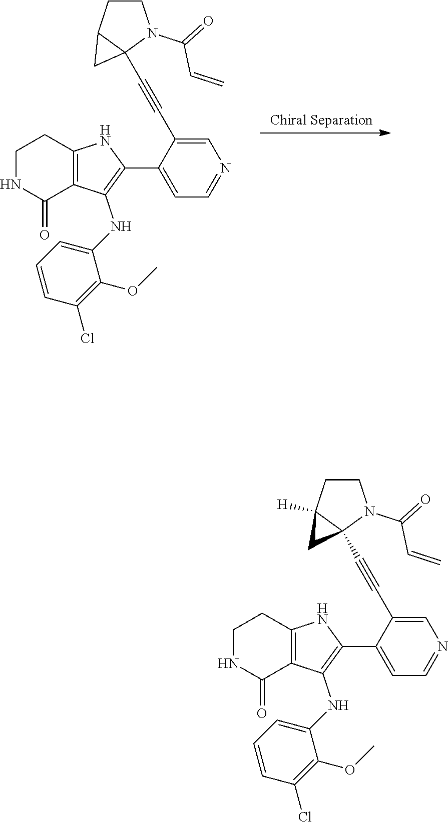 [SMILES: C=CC(=O)N1CCC2CC21C#Cc1cnccc1-c1[nH]c2c(c1Nc1cccc(Cl)c1OC)C(=O)NCC2 ; "Chiral Separation" ; C=CC(=O)N1CC[C@@H]2C[C@@]21C#Cc1cnccc1-c1[nH]c2c(c1Nc1cccc(Cl)c1OC)C(=O)NCC2]

The crude product (50 mg) was purified by Prep-CHIRAL-HPLC with the following conditions (Column: CHIRAL ART Amylose-SA, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 30% B to 30% B in 17 min; Wave Length: 220/254 nm; RT1(min): 8.301; RT2(min): 12.494; Sample Solvent: EtOH—HPLC; Injection Volume: 0.3 mL; Number Of Runs: 10) to afford 3-[(3-chloro-2-methoxyphenyl)amino]-2-(3-{2-[(1R,5S)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one (12 mg, 23.71%) as a yellow solid.

LC-MS: (M+H)⁺ found: 528.30.

¹H NMR (400 MHz, DMSO-d6) δ 11.55 (s, 1H), 8.43 (s, 1H), 8.27 (d, 1H), 7.39 (d, 1H), 6.73-6.59 (m, 5H), 6.15-6.09 (m, 1H), 5.66-5.49 (m, 1H), 4.10-3.85 (m, 2H), 3.46-3.42 (m, 2H), 3.25 (s, 3H), 2.90-2.87 (m, 3H), 2.40-2.28 (m, 1H), 2.15-2.04 (m, 1H), 1.78-1.62 (m, 1H), 0.93 (t, 1H).

### Example 129. 3-[(3-chloro-2-methoxyphenyl)amino]-2-(3-{2-[(1S,5R)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one (compound 202)

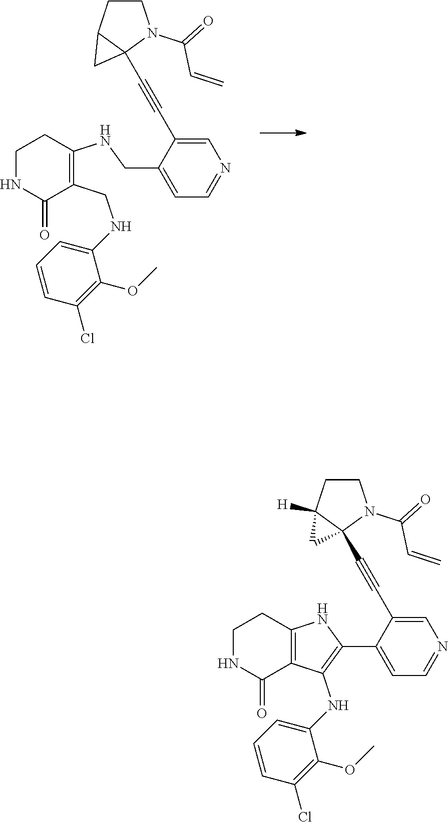 [SMILES: C=CC(=O)N1CCC2CC21C#Cc1cnccc1CNC1=C(CNc2cccc(Cl)c2OC)C(=O)NCC1 ; C=CC(=O)N1CC[C@H]2C[C@]21C#Cc1cnccc1-c1[nH]c2c(c1Nc1cccc(Cl)c1OC)C(=O)NCC2]

The crude product (50 mg) was purified by Prep-CHIRAL-HPLC with the following conditions (Column: CHIRAL ART Amylose-SA, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 30% B to 30% B in 17 min; Wave Length: 220/254 nm; RT1(min): 8.301; RT2(min): 12.494; Sample Solvent: EtOH—HPLC; Injection Volume: 0.3 mL; Number Of Runs: 10) to afford 3-[(3-chloro-2-methoxyphenyl)amino]-2-(3-{2-[(1S,5R)-2-(prop-2-enoyl)-2-azabicyclo[3.1.0]hexan-1-yl]ethynyl}pyridin-4-yl)-1H,5H,6H,7H-pyrrolo[3,2-c]pyridin-4-one (11.5 mg, 22.42%) as a yellow solid.

LC-MS: (M+H)⁺ found: 528.30.

¹H NMR (400 MHz, DMSO-d6) δ 11.54 (s, 1H), 8.43 (s, 1H), 8.25 (d, 1H), 7.35 (d, 1H), 6.73-6.59 (m, 5H), 6.16-6.10 (m, 1H), 5.66-5.39 (m, 1H), 4.09-3.84 (m, 2H), 3.46-3.42 (m, 2H), 3.25 (s, 3H), 2.90-2.87 (m, 3H), 2.40-2.28 (m, 1H), 2.15-2.04 (m, 1H), 1.68-1.66 (m, 1H), 0.93 (t, 1H).

### Example 130. 3-[(3-chloro-2-methoxyphenyl)amino]-2-(3-{2-[(2R)-2-methyl-1-(prop-2-enoyl)pyrrolidin-2-yl]ethynyl}pyridin-4-yl)-1H,5H,6H,7H-pyrrolo[3,2-c]pyridin-4-one (compound 210)

### 130.1. Synthesis of tert-butyl (R)-2-(hydroxymethyl)-2-methylpyrrolidine-1-carboxylate

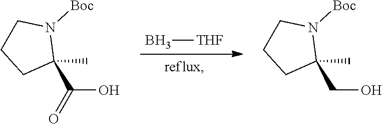

To a stirred solution of (2R)-1-(tert-butoxycarbonyl)-2-methylpyrrolidine-2-carboxylic acid (1 g, 4.36 mmol, 1.00 equiv) in THF (10.00 mL) was added BH₃(1M in THF) (8.72 mL, 8.724 mmol, 2 equiv) dropwise at 0° C. under nitrogen atmosphere. The resulting mixture was stirred for 1 h at 75° C. under nitrogen atmosphere. The reaction was quenched by the addition of MeOH (5.00 mL) at 0° C. The resulting mixture was concentrated under reduced pressure. The crude product was used in the next step directly without further purification.

LC-MS: (M+H−56)⁺ found: 160.3

### 130.2. Synthesis of tert-butyl (2R)-2-formyl-2-methylpyrrolidine-1-carboxylate

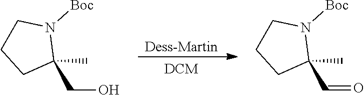

To a stirred mixture of tert-butyl (2R)-2-(hydroxymethyl)-2-methylpyrrolidine-1-carboxylate (1.1 g, 5.1 mmol, 1.00 equiv) in DCM (20.00 mL) was added Dess-Martin (2.60 g, 6.13 mmol, 1.20 equiv) dropwise at 0° C. under nitrogen atmosphere. The resulting mixture was stirred for 2 h at room temperature under nitrogen atmosphere. The reaction was quenched by the addition of Na₂SO₄ (sat.) (5.00 mL) at room temperature. The resulting mixture was extracted with CH₂Cl₂ (3×20 mL), dried over anhydrous Na₂SO₄. After filtration, the filtrate was concentrated under reduced pressure. The residue was purified by silica gel column chromatography, eluted with PE/EA (5:1) to afford tert-butyl (2R)-2-formyl-2-methylpyrrolidine-1-carboxylate (750 mg, 68.83%) as a colorless oil.

¹H NMR (400 MHz, Chloroform-d) δ 9.38 (d, 1H), 3.70-3.40 (m, 2H), 2.05-1.87 (m, 3H), 1.78-1.57 (m, 1H), 1.46 (s, 3H), 1.40 (d, J=12.8 Hz, 9H).

### 130.3. Synthesis of tert-butyl (2R)-2-ethynyl-2-methylpyrrolidine-1-carboxylate

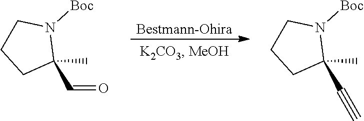

To a stirred solution of tert-butyl (2R)-2-formyl-2-methylpyrrolidine-1-carboxylate (700 mg, 3.28 mmol, 1.00 equiv) and K₂CO₃ (907 mg, 6.56 mmol, 2.00 equiv) in methanol (10.00 mL) was added Bestmann-Ohira (756 mg, 3.94 mmol, 1.20 equiv) dropwise at 0° C. under nitrogen atmosphere. The resulting mixture was stirred for 2 h at room temperature under nitrogen atmosphere. The reaction was quenched by the addition of Potassium sodium tartrate (sat.) (2 mL) at room temperature. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EA (5:1) to afford tert-butyl (2R)-2-ethynyl-2-methylpyrrolidine-1-carboxylate (600 mg, 87.35%) as a white oil.

¹H-NMR (300 MHz, Chloroform-d) δ 3.63-3.28 (m, 2H), 2.34-2.24 (m, 2H), 2.08-1.72 (m, 3H), 1.70 (s, 3H), 1.49 (s, 9H).

### 130.4. Synthesis of tert-butyl (2R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate

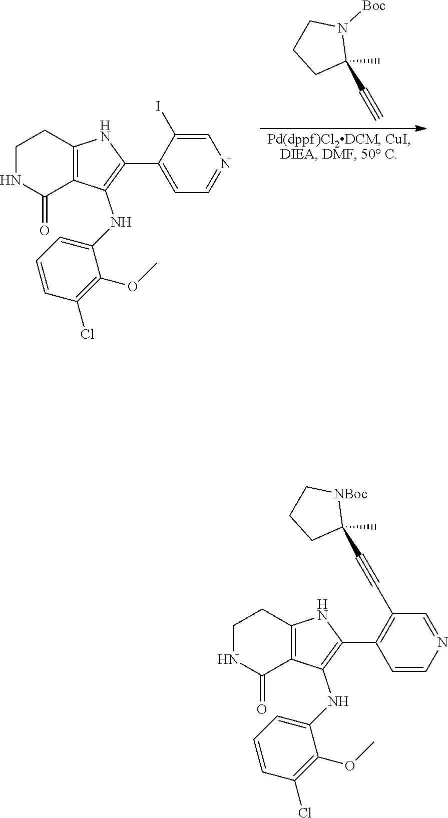 [SMILES: COc1c(Cl)cccc1Nc1c(-c2ccncc2I)[nH]c2c1C(=O)NCC2 ; C#C[C@@]1(C)CCCN1C(=O)OC(C)(C)C ; "Pd(dppf)Cl2·DCM, CuI,DIEA, DMF, 50° C." ; COc1c(Cl)cccc1Nc1c(-c2ccncc2C#C[C@@]2(C)CCCN2C(=O)OC(C)(C)C)[nH]c2c1C(=O)NCC2]

To a stirred mixture of 3-[(3-chloro-2-methoxyphenyl)amino]-2-(3-iodopyridin-4-yl)-1H,5H,6H,7H-pyrrolo[3,2-c]pyridin-4-one (150 mg, 0.30 mmol, 1.00 equiv), CuI (28 mg, 0.15 mmol, 0.50 equiv) and Pd(dppf)Cl₂CH₂Cl₂ (61 mg, 0.076 mmol, 0.25 equiv) in DMF (2.00 mL) were added tert-butyl (2R)-2-ethynyl-2-methylpyrrolidine-1-carboxylate (25 mg, 0.12 mmol, 3.00 equiv) and DIEA (195 mg, 1.51 mmol, 5.00 equiv) dropwise at room temperature under argon atmosphere. The resulting mixture was stirred for 2 h at 50° C. under argon atmosphere. The mixture was concentrated under reduced pressure. The residue was purified by reverse flash chromatography with the following conditions: column, C18 gel; mobile phase, MeCN in water, 10% to 70% gradient in 20 min; detector, UV 254 nm. This resulted in tert-butyl (2R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (160 mg, 91.6%) as a yellow solid.

LC-MS: (M+H)⁺ found: 576.05.

### 130.5. Synthesis of (R)-3-((3-chloro-2-methoxyphenyl)amino)-2-(3-((2-methylpyrrolidin-2-yl)ethynyl)pyridin-4-yl)-1,5,6,7-tetrahydro-4H-pyrrolo[3,2-c]pyridin-4-one

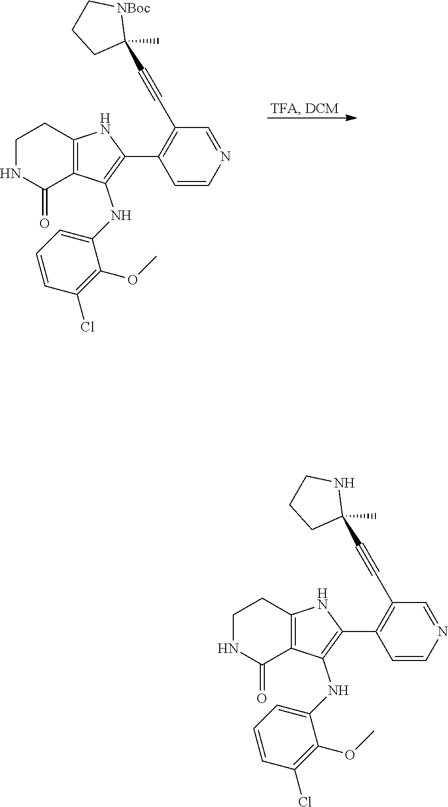

To a stirred solution of tert-butyl (2R)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (140 mg, 0.035 mmol, 1.00 equiv) in DCM (1.00 mL) was added TFA (3.00 mL) dropwise at room temperature under nitrogen atmosphere. The resulting mixture was stirred for 1 h at room temperature under nitrogen atmosphere. The mixture was concentrated under reduced pressure. The crude product was used in the next step directly without further purification.

LC-MS: (M+H)⁺ found: 476.1

### 130.6. Synthesis of 3-[(3-chloro-2-methoxyphenyl)amino]-2-(3-{2-[(2R)-2-methyl-1-(prop-2-enoyl)pyrrolidin-2-yl]ethynyl}pyridin-4-yl)-1H,5H,6H,7H-pyrrolo[3,2-c]pyridin-4-one

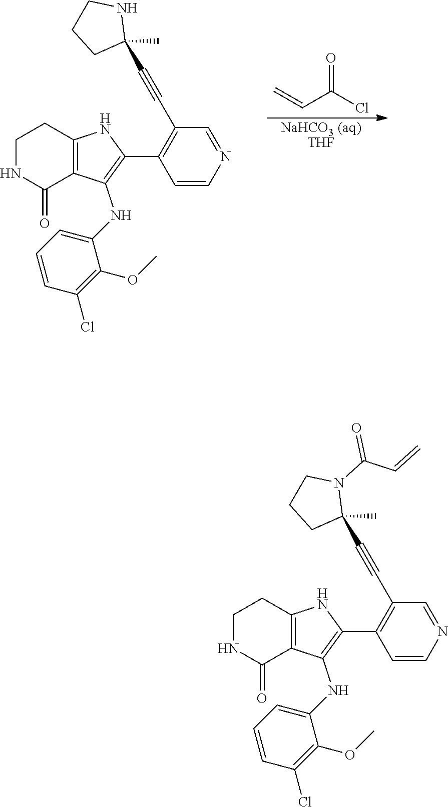

To a stirred solution of 3-[(3-chloro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (100 mg, 0.21 mmol, 1.00 equiv) and NaHCO₃(sat.) (3.00 mL) in THF (3.00 mL) was added acryloyl chloride (19 mg, 0.21 mmol, 1.00 equiv) dropwise at 0° C. under nitrogen atmosphere. The resulting mixture was stirred for 1 h at room temperature under nitrogen atmosphere. The resulting mixture was extracted with CH₂Cl₂/MeOH(10/1) (3×20 mL). The combined organic layers were washed with brine (3×20 mL), dried over anhydrous Na₂SO₄. After filtration, the filtrate was concentrated under reduced pressure. The crude product (120 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 35% B to 55% B in 10 min, 55% B; Wave Length: 254/220 nm; RT1(min): 9.67; Number Of Runs: 0) to afford 3-[(3-chloro-2-methoxyphenyl)amino]-2-(3-{2-[(2R)-2-methyl-1-(prop-2-enoyl)pyrrolidin-2-yl]ethynyl}pyridin-4-yl)-1H,5H,6H,7H-pyrrolo[3,2-c]pyridin-4-one (17 mg, 15.00%) as a yellow solid.

LC-MS: (M+H)⁺ found: 530.30

¹HTEM-NMR (400 MHz, DMSO-d6) δ 11.35 (s, 1H), 8.50 (s, 1H), 8.20 (d, 2H), 7.46-7.29 (m, 2H), 6.82-6.54 (m, 4H), 6.22-6.10 (m, 2H), 5.65 (d, 1H), 3.89 (s, 3H), 3.68 (d, 2H), 3.47-3.40 (m, 2H), 3.10-3.02 (m, 2H), 2.44-2.35 (m, 1H), 2.19-2.09 (m, 1H), 2.05-1.93 (m, 2H), 1.69 (s, 3H).

### Example 131. 3-[(3-chloro-2-methoxyphenyl)amino]-2-(3-{2-[(2S)-2-methyl-1-(prop-2-enoyl)pyrrolidin-2-yl]ethynyl}pyridin-4-yl)-1H,5H,6H,7H-pyrrolo[3,2-c]pyridin-4-one)(compound 209)

### 131.1. Synthesis of tert-butyl (S)-2-(hydroxymethyl)-2-methylpyrrolidine-1-carboxylate

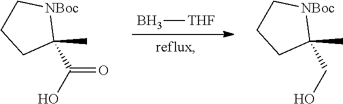

To a stirred solution of (2S)-1-(tert-butoxycarbonyl)-2-methylpyrrolidine-2-carboxylic acid (1 g, 4.36 mmol, 1.00 equiv) in THF (8.00 mL) were added BH₃(1M in THF, 8.7 mL, 8.7 mmol, 2.0 equiv) at room temperature. The resulting mixture was stirred for 1 h at 75° C. under N₂ atmosphere. The reaction was monitored by LCMS. The reaction was quenched by the addition of MeOH (2.00 mL) at 0° C. The resulting mixture was concentrated under vacuum. The crude product was used in the next step directly without further purification.

LC-MS: (M+H−56)⁺ found: 160.15.

### 131.2. Synthesis of tert-butyl (2S)-2-formyl-2-methylpyrrolidine-1-carboxylate

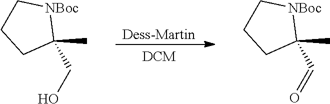

To a stirred solution of tert-butyl (2S)-2-(hydroxymethyl)-2-methylpyrrolidine-1-carboxylate (938 mg, 4.36 mmol, 1.00 equiv) in DCM (10.00 mL) were added Dess-Martin (2.22 g, 5.23 mmol, 1.20 equiv) at 0° C. The resulting mixture was stirred for 2h at room temperature under nitrogen atmosphere. The reaction was monitored by TLC. The resulting mixture was concentrated under vacuum. The residue was purified by silica gel column chromatography, eluted with EA/PE (20%) to afford tert-butyl (2S)-2-formyl-2-methylpyrrolidine-1-carboxylate (640 mg, 68.88%) as a colorless oil.

### 131.3. Synthesis of tert-butyl (2S-2-ethynyl-2-methylpyrrolidine-1-carboxylate

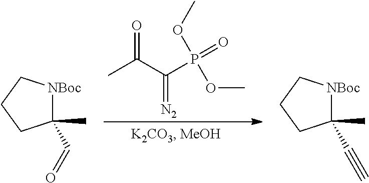

To a stirred solution of tert-butyl (2S)-2-formyl-2-methylpyrrolidine-1-carboxylate (600 mg, 2.81 mmol, 1.00 equiv) in MeOH (6.00 mL) was added K₂CO₃ (778 mg, 5.63 mmol, 2 equiv), dimethyl (1-diazo-2-oxopropyl)phosphonate (648 mg, 3.38 mmol, 1.20 equiv) dropwise at 0° C. under nitrogen atmosphere. The resulting mixture was stirred for 2h at room temperature under nitrogen atmosphere. The reaction was quenched by the addition of potassium sodium tartrate (sat.) (5.00 mL) at 0° C. The resulting mixture was stirred for 0.5h at room temperature. The reaction was monitored by TLC and H-NMR. The resulting mixture was extracted with ethyl acetate (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with EA/PE (5%) to afford tert-butyl (2S)-2-ethynyl-2-methylpyrrolidine-1-carboxylate (640 mg, 68.88%) as a colorless oil.

¹H NMR (400 MHz, Chloroform-d) δ 3.55 (s, 1H), 3.41-3.35 (m, 1H), 2.30-2.27 (m, 2H), 1.99-1.90 (m, 2H), 1.83-1.77 (m, 1H), 1.63 (s, 3H), 1.49 (s, 9H).

### 131.4. Synthesis of tert-butyl (2S)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate

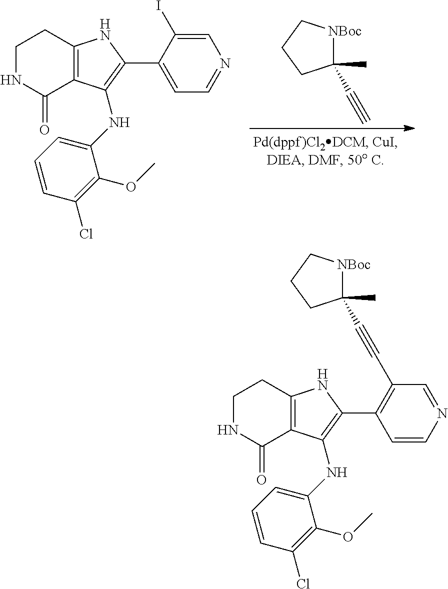 [SMILES: COc1c(Cl)cccc1Nc1c(-c2ccncc2I)[nH]c2c1C(=O)NCC2 ; C#C[C@]1(C)CCCN1C(=O)OC(C)(C)C ; COc1c(Cl)cccc1Nc1c(-c2ccncc2C#C[C@]2(C)CCCN2C(=O)OC(C)(C)C)[nH]c2c1C(=O)NCC2]

To a solution of 3-[(3-chloro-2-methoxyphenyl)amino]-2-(3-iodopyridin-4-yl)-1H,5H,6H,7H-pyrrolo[3,2-c]pyridin-4-one (150 mg, 0.30 mmol, 1.00 equiv) and tert-butyl (2S)-2-ethynyl-2-methylpyrrolidine-1-carboxylate (127 mg, 0.61 mmol, 2.00 equiv) in DMF (2.00 mL) were added DIEA (98 mg, 0.76 mmol, 2.50 equiv), CuI (29 mg, 0.15 mmol, 0.50 equiv), Pd(dppf)Cl₂·CH₂Cl₂ (62 mg, 0.08 mmol, 0.25 equiv). The mixture was stirred for 2 h at 50° C. under nitrogen atmosphere. The resulting mixture was concentrated under reduced pressure. Desired product could be detected by LCMS. The residue was purified by reverse flash chromatography with the following conditions: column, C18 gel; mobile phase, ACN in water, 0% to 100% gradient in 30 min; detector, UV 254 nm to afford tert-butyl (2S)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (155 mg, 84.30%) as a yellow solid.

LC-MS: (M+H)⁺ found: 576.10.

### 131.5. Synthesis of (S)-3-((3-chloro-2-methoxyphenyl)amino)-2-(3-((2-methylpyrrolidin-2-yl)ethynyl)pyridin-4-yl)-1,5,6,7-tetrahydro-4H-pyrrolo[3,2-c]pyridin-4-one

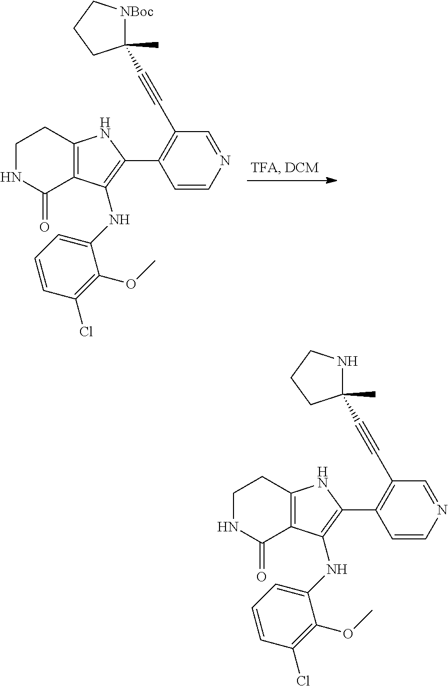 [SMILES: COc1c(Cl)cccc1Nc1c(-c2ccncc2C#C[C@]2(C)CCCN2C(=O)OC(C)(C)C)[nH]c2c1C(=O)NCC2 ; COc1c(Cl)cccc1Nc1c(-c2ccncc2C#C[C@]2(C)CCCN2)[nH]c2c1C(=O)NCC2]

To a solution of tert-butyl (2S)-2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (60 mg, 0.10 mmol, 1.00 equiv) in DCM (1.00 mL) were added and TFA (1.00 mL) at room temperature. The resulting mixture was stirred for 0.5h at room temperature. The reaction was monitored by LCMS. The resulting mixture was concentrated under vacuum. The crude product was used in the next step directly without further purification.

LC-MS: (M+H)⁺ found: 476.10.

### 131.6. Synthesis of 3-[(3-chloro-2-methoxyphenyl)amino]-2-(3-{2-[(2S)-2-methyl-1-(prop-2-enoyl)pyrrolidin-2-yl]ethynyl}pyridin-4-yl)-1H,5H,6H,7H-pyrrolo[3,2-c]pyridin-4-one)

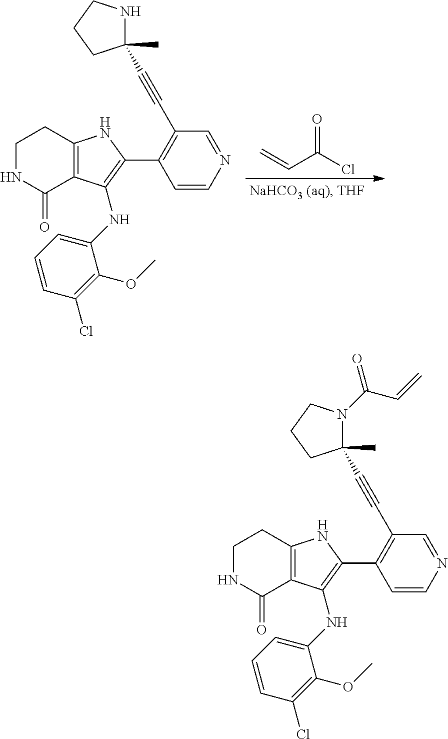

To a stirred solution of 3-[(3-chloro-2-methoxyphenyl)amino]-2-(3-{2-[(2S)-2-methylpyrrolidin-2-yl] ethynyl}pyridin-4-yl)-1H,5H,6H,7H-pyrrolo[3,2-c]pyridin-4-one (60 mg, 0.17 mmol, 1.00 equiv) in THF (2.00 mL) and NaHCO₃(sat.) (2.00 mL) was added acryloyl chloride (10 mg, 0.11 mmol, 0.9 equiv) dropwise at 0° C. under nitrogen atmosphere. The resulting mixture was stirred for 1 h at room temperature under nitrogen atmosphere. The reaction was monitored by LCMS. Desired product could be detected by LCMS. The resulting mixture was concentrated under vacuum. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 35% B to 58% B in 9 min, 58% B; Wave Length: 254/220 nm; RT1(min): 8.85; Number Of Runs: 0) to afford 3-[(3-chloro-2-methoxyphenyl)amino]-2-(3-{2-[(2S)-2-methyl-1-(prop-2-enoyl)pyrrolidin-2-yl]ethynyl}pyridin-4-yl)-1H,5H,6H,7H-pyrrolo[3,2-c]pyridin-4-one) (8.1 mg, 11.81%) as an off-white solid.

LC-MS: (M+H)⁺ found: 530.00

¹HTEM NMR (400 MHz, DMSO-d6) δ 11.36 (s, 1H), 8.49 (s, 1H), 8.22-8.20 (m, 1H), 7.35-7.34 (m, 2H), 6.77 (s, 1H), 6.67-6.61 (m, 3H), 6.27-6.00 (m, 2H), 5.67 (d, J=9.6 Hz, 1H), 3.90 (s, 3H), 3.75-3.60 (m, 2H), 3.46-3.43 (m, 2H), 3.08-2.98 (m, 2H), 2.49-2.47 (m, 1H), 2.18-2.07 (m, 1H), 2.03-1.94 (m, 2H), 1.71 (s, 3H).

### Example 132. 3-[(3-chloro-2-methoxyphenyl)amino]-2-(3-{2-[(6R)-5-(prop-2-enoyl)-5-azaspiro[2.4]heptan-6-yl]ethynyl}pyridin-4-yl)-1H,5H,6H,7H-pyrrolo[3,2-c]pyridin-4-one (compound 234)

### 132.1. Synthesis of tert-butyl (R)-6-(hydroxymethyl)-5-azaspiro[2.4]heptane-5-carboxylate

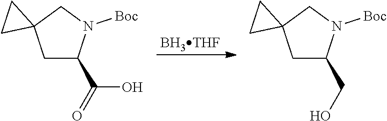 [SMILES: CC(C)(C)OC(=O)N1CC2(CC2)C[C@@H]1C(=O)O ; CC(C)(C)OC(=O)N1CC2(CC2)C[C@@H]1CO ; "BH3·THF"]

To a solution of (6S)-5-(tert-butoxycarbonyl)-5-azaspiro[2.4]heptane-6-carboxylic acid (2.40 g, 9.95 mmol, 1.00 equiv) in THF was added BH₃ (15.00 mL, 15.00 mmoL, 1.5 equiv, 1M in THF) dropwise at 0° C. The resulting mixture was stirred for 2 h at room temperature. The reaction was quenched by the addition of MeOH (15.00 mL) at 0° C. The resulting mixture was concentrated under reduced pressure to obtain tert-butyl (R)-6-(hydroxymethyl)-5-azaspiro[2.4]heptane-5-carboxylate (2.6 g, crude) as a colorless oil.

### 132.2. Synthesis of tert-butyl (6R)-6-formyl-5-azaspiro[2.4]heptane-5-carboxylate

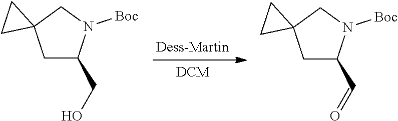 [SMILES: CC(C)(C)OC(=O)N1CC2(CC2)C[C@@H]1CO ; CC(C)(C)OC(=O)N1CC2(CC2)C[C@@H]1C=O]

To a solution of tert-butyl (6R)-6-(hydroxymethyl)-5-azaspiro[2.4]heptane-5-carboxylate (2.30 g, 10.11 mmol, 1.00 equiv) in DCM (25.00 mL) was added Dess-Martin reagent (5.15 g, 12.14 mmol, 1.20 equiv) in portions at 0° C. The resulting mixture was stirred for 2.5 h at room temperature. The reaction was quenched by the addition of sat. Na₂S203 (aq.) (15 mL) at 0° C. The resulting mixture was extracted with CH₂Cl₂ (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (5:1) to afford tert-butyl (6R)-6-formyl-5-azaspiro[2.4]heptane-5-carboxylate (1.99 g, 87.30%) as a light yellow oil.

LC-MS: (M+H−56)⁺ found: 169.95.

¹H NMR (300 MHz, Chloroform-d) δ 9.66-9.58 (m, 1H), 4.36-4.17 (m, 1H), 3.48-3.41 (m, 1H), 3.37-3.22 (m, 1H), 2.15-2.08 (m, 1H), 1.88-1.82 (m, 1H), 1.48-1.45 (m, 9H), 0.66-0.49 (m, 4H).

### 132.3. Synthesis of tert-butyl (6R)-6-ethynyl-5-azaspiro[2.4]heptane-5-carboxylate

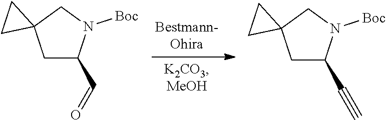 [SMILES: CC(C)(C)OC(=O)N1CC2(CC2)C[C@@H]1C=O ; C#C[C@H]1CC2(CC2)CN1C(=O)OC(C)(C)C]

To a stirred mixture of tert-butyl (6R)-6-formyl-5-azaspiro[2.4]heptane-5-carboxylate (1.2 g, 5.327 mmol, 1 equiv) and K₂CO₃ (1.47 g, 10.65 mmol, 2.0 equiv) in MeOH (8.00 mL) was added dimethyl (1-diazo-2-oxopropyl)phosphonate (1.23 g, 6.392 mmol, 1.2 equiv) dropwise at 0° C. The resulting mixture was stirred for overnight at room temperature. The reaction was quenched by the addition of the solution of Potassium sodium tartrate tetrahydrate (aq., 20 mL) at room temperature. The resulting mixture was extracted with EtOAc (3×15 mL). The combined organic layers were washed with brine (1×30 mL), dried over anhydrous Na₂SO₄. After filtration, the filtrate was concentrated under reduced pressure. The residue was purified by silica gel with PE/EA (5:1). This resulted in tert-butyl (6R)-6-ethynyl-5-azaspiro[2.4]heptane-5-carboxylate (900 mg, 76.35%) as a yellow oil.

LC-MS: (M+H−56)⁺ found: 166.3.

¹H-NMR (400 MHz, Chloroform-d) δ 4.61-4.52 (m, 1H), 3.40 (d, 1H), 3.19 (s, 1H), 2.32-2.24 (m, 2H), 1.74-1.71 (m, 1H), 1.48 (s, 9H), 0.71-0.67 (m, 2H), 0.58-0.54 (m, 2H).

### 132.4. Synthesis of tert-butyl (6R)-6-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-5-azaspiro[2.4]heptane-5-carboxylate

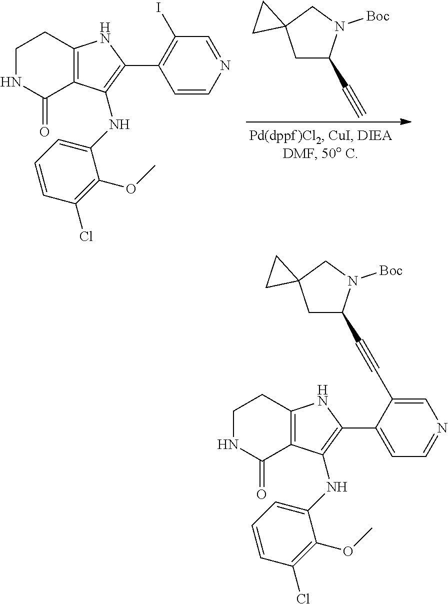

To a stirred solution of 3-[(3-chloro-2-methoxyphenyl)amino]-2-(3-iodopyridin-4-yl)-1H,5H,6H,7H-pyrrolo[3,2-c]pyridin-4-one (150 mg, 0.30 mmol, 1.00 equiv) and Pd(dppf)Cl₂CH₂Cl₂ (61 mg, 0.076 mmol, 0.25 equiv) and CuI (28 mg, 0.15 mmol, 0.50 equiv) in DMF were added tert-butyl (6R)-6-ethynyl-5-azaspiro[2.4]heptane-5-carboxylate (335 mg, 1.51 mmol, 5.00 equiv) and DIEA (195 mg, 1.51 mmol, 5.00 equiv) at room temperature under argon atmosphere. The resulting suspension was backfilled with argon three times and stirred for 2h at 50° C. LCMS confirmed completion of reaction and desired product was observed. The resulting mixture was concentrated under reduced pressure. The residue was purified by reverse phase flash to afford tert-butyl (6R)-6-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-5-azaspiro[2.4]heptane-5-carboxylate (150 mg, 84.12%) as a yellow solid.

LC-MS: (M+H)⁺ found: 588.15.

### 132.5. Synthesis of 2-(3-{2-[(6R)-5-azaspiro[2.4]heptan-6-yl]ethynyl}pyridin-4-yl)-3-[(3-chloro-2-methoxyphenyl)amino]-1H,5H,6H,7H-pyrrolo[3,2-c]pyridin-4-one

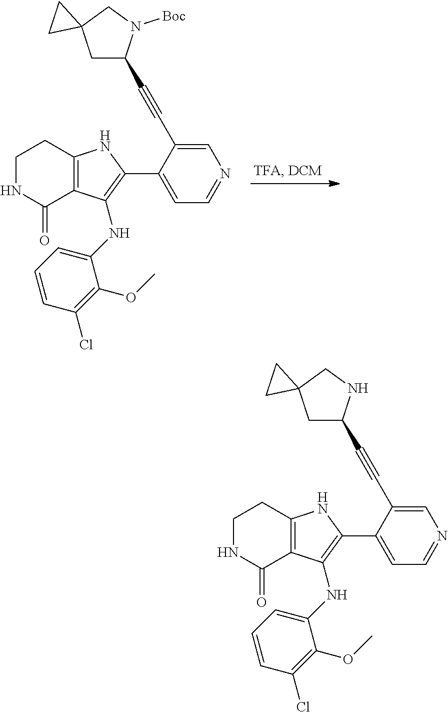 [SMILES: COc1c(Cl)cccc1Nc1c(-c2ccncc2C#C[C@H]2CC3(CC3)CN2C(=O)OC(C)(C)C)[nH]c2c1C(=O)NCC2 ; COc1c(Cl)cccc1Nc1c(-c2ccncc2C#C[C@H]2CC3(CC3)CN2)[nH]c2c1C(=O)NCC2]

To a stirred mixture of tert-butyl (2S)-2-{[(4-{3-[(2-chloro-3-fluorophenyl)amino]-4-oxo-1H,5H,6H,7H-pyrrolo[3,2-c]pyridin-2-yl}pyridin-3-yl)oxy]methyl}pyrrolidine-1-carboxylate (140 mg, 0.252 mmol, 1 equiv) in DCM (3 mL) was added TFA (1 mL) at room temperature under nitrogen atmosphere. The resulting mixture was stirred for 1 h and concentrated under reduced pressure to afford 2-(3-{2-[(6R)-5-azaspiro[2.4]heptan-6-yl]ethynyl}pyridin-4-yl)-3-[(3-chloro-2-methoxyphenyl)amino]-1H,5H,6H,7H-pyrrolo[3,2-c]pyridin-4-one (200 mg, crude) as a red oil.

LC-MS: (M+H)⁺ found: 488.1

### 132.6. Synthesis of 3-[(3-chloro-2-methoxyphenyl)amino]-2-(3-{2-[(6R)-5-(prop-2-enoyl)-5-azaspiro[2.4]heptan-6-yl]ethynyl}pyridin-4-yl)-1H,5H,6H,7H-pyrrolo[3,2-c]pyridin-4-one

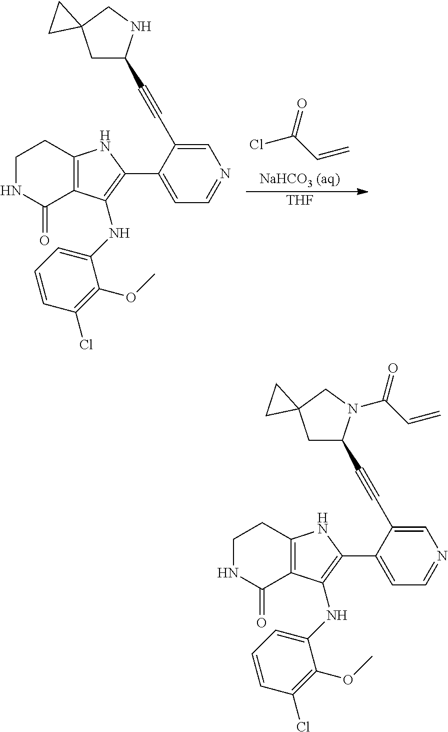

To a stirred solution of 2-(3-{2-[(6R)-5-azaspiro[2.4]heptan-6-yl]ethynyl}pyridin-4-yl)-3-[(3-chloro-2-methoxyphenyl)amino]-1H,5H,6H,7H-pyrrolo[3,2-c]pyridin-4-one (110 mg, 0.22 mmol, 1 equiv) and sat. NaHCO₃(aq.) (1.50 mL) in THF (1.50 mL) was added acryloyl chloride (22 mg, 0.22 mmol, 1.00 equiv) dropwise at 0° C. The resulting mixture was stirred for 1.5 h at room temperature. The resulting mixture was extracted with EtOAc (3×5 mL). The combined organic layers were washed with brine (1×30 mL), dried over anhydrous Na₂SO₄. After filtration, the filtrate was concentrated under reduced pressure. The crude product (130 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 28% B to 49% B in 8 min, 49% B; Wave Length: 254/220 nm; RT1(min): 8; Number Of Runs: 0) to afford 3-[(3-chloro-2-methoxyphenyl)amino]-2-(3-{2-[(6R)-5-(prop-2-enoyl)-5-azaspiro[2.4]heptan-6-yl]ethynyl}pyridin-4-yl)-1H,5H,6H,7H-pyrrolo[3,2-c]pyridin-4-one (33.6 mg, 26.66%) as a yellow solid.

LC-MS: (M+H)⁺ found: 542.10.

¹H NMR (400 MHz, Chloroform-d) δ 11.15 (s, 1H), 8.53 (s, 1H), 8.13 (d, 1H), 7.77 (s, 1H), 7.42 (d, 1H), 6.73-6.71 (m, 1H), 6.60 (t, 1H), 6.41 (d, 2H), 6.24-6.21 (m, 1H), 5.77 (t, 1H), 5.27 (s, 1H), 5.04-5.01 (m, 1H), 4.07 (s, 3H), 3.67-3.50 (m, 4H), 3.31-3.27 (m, 2H), 2.34-2.29 (m, 1H), 2.19-2.14 (m, 1H), 0.89-0.81 (m, 2H), 0.74-0.70 (m, 2H).

### Example 133. 3-[(3-chloro-2-methoxyphenyl)amino]-2-(3-{2-[(2S)-3,3-dimethyl-1-(prop-2-enoyl)azetidin-2-yl]ethynyl}pyridin-4-yl)-1H,5H,6H,7H-pyrrolo[3,2-c]pyridin-4-one (compound 233)

### 133.1. Synthesis of tert-butyl 2-ethynyl-3,3-dimethylazetidine-1-carboxylate

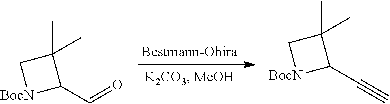

To a stirred solution of tert-butyl 2-formyl-3,3-dimethylazetidine-1-carboxylate (200 mg, 0.94 mmol, 1.00 equiv) in methanol (10 mL) was added K₂CO₃ (259 mg, 1.88 mmol, 2.00 equiv) and dimethyl (1-diazo-2-oxopropyl)phosphonate (216 mg, 1.13 mmol, 1.20 equiv) dropwise at 0° C. under nitrogen atmosphere. The resulting mixture was stirred for 2 h at room temperature under nitrogen atmosphere. The reaction was quenched by the addition of sat. Potassium sodium tartrate (5.00 mL) at room temperature. The resulting mixture was extracted with EtOAc (3×20 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EA (5:1) to afford tert-butyl 2-ethynyl-3,3-dimethylazetidine-1-carboxylate (160 mg, 81.52%) as a white oil.

¹H NMR (300 MHz, Chloroform-d) δ 4.37 (d, 1H), 3.54 (s, 2H), 2.53 (d, 1H), 1.48 (s, 9H), 1.31 (s, 3H), 1.27 (s, 3H).

### 133.2. Synthesis of tert-butyl 2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-3,3-dimethylazetidine-1-carboxylate

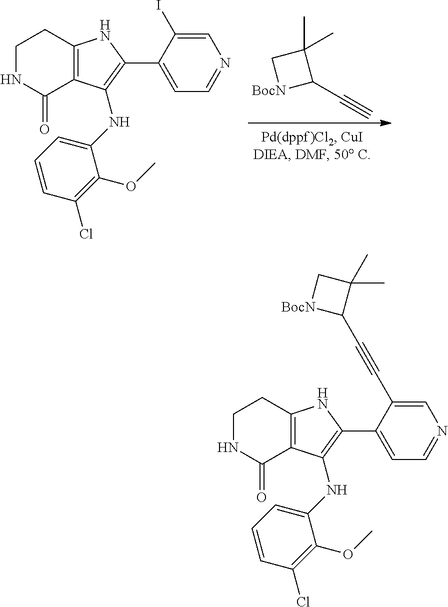 [SMILES: COc1c(Cl)cccc1Nc1c(-c2ccncc2I)[nH]c2c1C(=O)NCC2 ; C#CC1N(C(=O)OC(C)(C)C)CC1(C)C ; COc1c(Cl)cccc1Nc1c(-c2ccncc2C#CC2N(C(=O)OC(C)(C)C)CC2(C)C)[nH]c2c1C(=O)NCC2]

To a stirred mixture of 3-[(3-chloro-2-methoxyphenyl)amino]-2-(3-iodopyridin-4-yl)-1H,5H,6H,7H-pyrrolo[3,2-c]pyridin-4-one (400 mg, 0.81 mmol, 1.00 equiv), CuI (76 mg, 0.40 mmol, 0.50 equiv) and Pd(dppf)Cl₂CH₂Cl₂ (164 mg, 0.20 mmol, 0.25 equiv) in DMF (3.00 mL) was added tert-butyl 2-ethynyl-3,3-dimethylazetidine-1-carboxylate (507 mg, 2.42 mmol, 3.00 equiv) and DIEA (522 mg, 4.04 mmol, 5.00 equiv) dropwise at room temperature under argon atmosphere. The resulting mixture was stirred for 2 h at 50° C. under argon atmosphere. The residue was purified by reverse flash chromatography with the following conditions: column, silica gel; mobile phase, MeCN in water, 10% to 70% gradient in 20 min; detector, UV 254 nm. This resulted in tert-butyl 2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-3,3-dimethylazetidine-1-carboxylate (270 mg, 57.97%) as a yellow solid.

LC-MS: (M+H)⁺ found: 576.20.

### 133.3. Synthesis of 3-((3-chloro-2-methoxyphenyl)amino)-2-(3-((3,3-dimethylazetidin-2-yl)ethynyl)pyridin-4-yl)-1,5,6,7-tetrahydro-4H-pyrrolo[3,2-c]pyridin-4-one

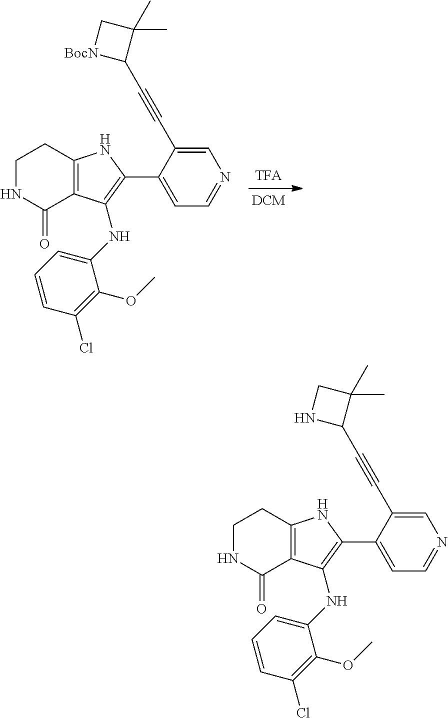 [SMILES: COc1c(Cl)cccc1Nc1c(-c2ccncc2C#CC2N(C(=O)OC(C)(C)C)CC2(C)C)[nH]c2c1C(=O)NCC2 ; COc1c(Cl)cccc1Nc1c(-c2ccncc2C#CC2NCC2(C)C)[nH]c2c1C(=O)NCC2]

To a stirred solution of tert-butyl 2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-3,3-dimethylazetidine-1-carboxylate (270 mg, 0.47 mmol, 1.00 equiv) in DCM (6.00 mL) was added TFA (2.00 mL) dropwise at room temperature under nitrogen atmosphere. The resulting mixture was stirred for 1 h at room temperature under nitrogen atmosphere. The resulting mixture was concentrated under reduced pressure. The crude product was used in the next step directly without further purification.

LC-MS: (M+H)⁺ found: 476.2.

### 133.4. Synthesis of 3-[(3-chloro-2-methoxyphenyl)amino]-2-(3-{2-[3,3-dimethyl-1-(prop-2-enoyl)azetidin-2-yl]ethynyl}pyridin-4-yl)-1H,5H,6H,7H-pyrrolo[3,2-c]pyridin-4-one

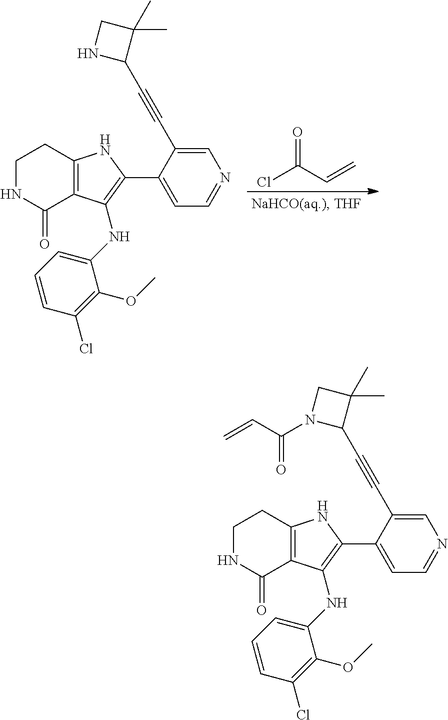

To a stirred mixture of 3-[(3-chloro-2-methoxyphenyl)amino]-2-{3-[2-(3,3-dimethylazetidin-2-yl)ethynyl]pyridin-4-yl}-1H,5H,6H,7H-pyrrolo[3,2-c]pyridin-4-one (200 mg, 0.42 mmol, 1.00 equiv) in THF (6 mL) and NaHCO₃(6 mL) was added acryloyl chloride (38 mg, 0.42 mmol, 1.00 equiv) dropwise at 0° C. under nitrogen atmosphere. The resulting mixture was stirred for 1 h at room temperature under nitrogen atmosphere. The resulting mixture was concentrated under reduced pressure. The residue was purified by silica gel column chromatography, eluted with CH₂Cl₂/MeOH (20:1) to afford the crude product, the crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 28% B to 52% B in 9 min, 52% B; Wave Length: 254/220 nm; RT1(min): 8.85; Number Of Runs: 0) to afford 3-[(3-chloro-2-methoxyphenyl)amino]-2-(3-{2-[3,3-dimethyl-1-(prop-2-enoyl)azetidin-2-yl]ethynyl}pyridin-4-yl)-1H,5H,6H,7H-pyrrolo[3,2-c]pyridin-4-one (60 mg, 26.94%) as a yellow solid.

LC-MS: (M+H)⁺ found: 530.35

### 133.5. Synthesis of 3-[(3-chloro-2-methoxyphenyl)amino]-2-(3-{2-[(2S)-3,3-dimethyl-1-(prop-2-enoyl)azetidin-2-yl]ethynyl}pyridin-4-yl)-1H,5H,6H,7H-pyrrolo[3,2-c]pyridin-4-one

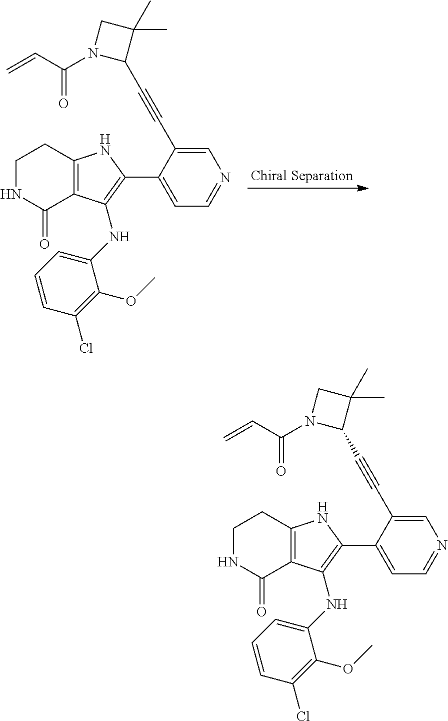 [SMILES: C=CC(=O)N1CC(C)(C)C1C#Cc1cnccc1-c1[nH]c2c(c1Nc1cccc(Cl)c1OC)C(=O)NCC2 ; C=CC(=O)N1CC(C)(C)[C@@H]1C#Cc1cnccc1-c1[nH]c2c(c1Nc1cccc(Cl)c1OC)C(=O)NCC2]

The crude product (60 mg) was purified by Prep-CHIRAL-HPLC with the following conditions (Column: CHIRAL ART Amylose-SA, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 25% B to 25% B in 27 min; Wave Length: 220/254 nm; RT1(min): 15.594; RT2(min): 22.091; Sample Solvent: EtOH—HPLC; Injection Volume: 1.25 mL; Number Of Runs: 2) to afford 3-[(3-chloro-2-methoxyphenyl)amino]-2-(3-{2-[(2S)-3,3-dimethyl-1-(prop-2-enoyl)azetidin-2-yl]ethynyl}pyridin-4-yl)-1H,5H,6H,7H-pyrrolo[3,2-c]pyridin-4-one (22.8 mg, 45.42%) as a yellow solid.

LC-MS: (M+H)⁺ found: 530.10

¹H NMR (300 MHz, Chloroform-d) δ 10.97 (s, 1H), 8.56 (s, 1H), 8.12 (d, 1H), 7.86 (s, 1H), 7.41 (d, 1H), 6.78-6.69 (m, 1H), 6.61 (t, 1H), 6.43-6.31 (m, 1H), 6.28-6.13 (m, 2H), 5.82-5.72 (m, 1H), 5.26 (s, 1H), 4.86 (s, 1H), 4.07 (s, 3H), 3.98 (d, 2H), 3.64-3.52 (m, 2H), 3.30-3.19 (m, 2H), 1.54 (s, 3H), 1.45 (s, 3H).

### Example 134. 3-[(3-chloro-2-methoxyphenyl)amino]-2-(3-{2-[(2R)-3,3-dimethyl-1-(prop-2-enoyl)azetidin-2-yl]ethynyl}pyridin-4-yl)-1H,5H,6H,7H-pyrrolo[3,2-c]pyridin-4-one (compound 232)

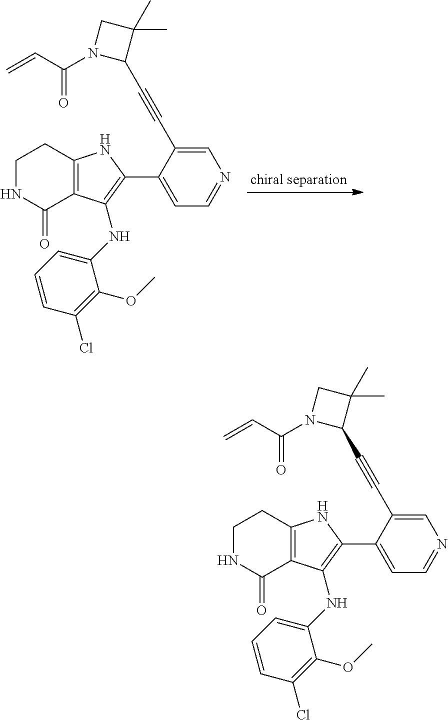 [SMILES: C=CC(=O)N1CC(C)(C)C1C#Cc1cnccc1-c1[nH]c2c(c1Nc1cccc(Cl)c1OC)C(=O)NCC2 ; C=CC(=O)N1CC(C)(C)[C@H]1C#Cc1cnccc1-c1[nH]c2c(c1Nc1cccc(Cl)c1OC)C(=O)NCC2]

The crude product (60 mg) was purified by Prep-CHIRAL-HPLC with the following conditions (Column: CHIRAL ART Amylose-SA, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 25% B to 25% B in 27 min; Wave Length: 220/254 nm; RT1(min): 15.594; RT2(min): 22.091; Sample Solvent: EtOH—HPLC; Injection Volume: 1.25 mL; Number Of Runs: 2) to afford 3-[(3-chloro-2-methoxyphenyl)amino]-2-(3-{2-[(2R)-3,3-dimethyl-1-(prop-2-enoyl)azetidin-2-yl]ethynyl}pyridin-4-yl)-1H,5H,6H,7H-pyrrolo[3,2-c]pyridin-4-one (22.7 mg, 45.31%) as a yellow solid.

LC-MS: (M+H)⁺ found: 530.10

¹H NMR (300 MHz, Chloroform-d) δ 10.96 (s, 1H), 8.56 (s, 1H), 8.13 (d, 1H), 7.84 (s, 1H), 7.41 (d, 1H), 6.78-6.69 (m, 1H), 6.61 (t, 1H), 6.43-6.31 (m, 1H), 6.28-6.13 (m, 2H), 5.81-5.71 (m, 1H), 5.26 (s, 1H), 4.86 (s, 1H), 4.07 (s, 3H), 3.98 (d, 2H), 3.67-3.54 (m, 2H), 3.29-3.19 (m, 2H), 1.54 (s, 3H), 1.45 (s, 3H).

### Example 135. 3-[(3-chloro-2-methoxyphenyl)amino]-2-(3-{2-[(2R)-1-(prop-2-enoyl)piperidin-2-yl]ethynyl}pyridin-4-yl)-1H,5H,6H,7H-pyrrolo[3,2-c]pyridin-4-one (compound 231)

### 135.1. Synthesis of tert-butyl2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]piperidine-1-carboxylate

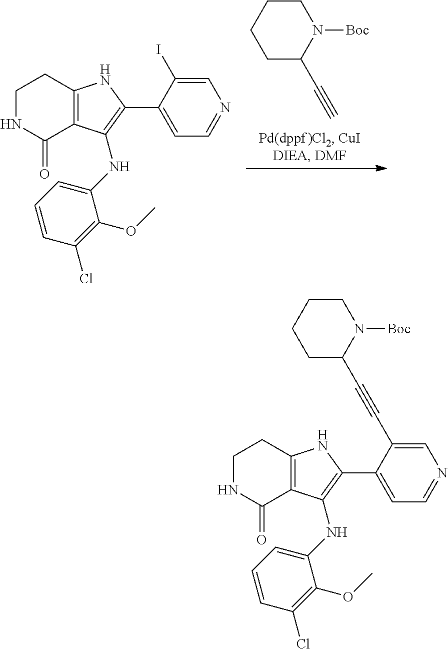 [SMILES: COc1c(Cl)cccc1Nc1c(-c2ccncc2I)[nH]c2c1C(=O)NCC2 ; C#CC1CCCCN1C(=O)OC(C)(C)C ; COc1c(Cl)cccc1Nc1c(-c2ccncc2C#CC2CCCCN2C(=O)OC(C)(C)C)[nH]c2c1C(=O)NCC2]

To a stirred mixture of 3-[(3-chloro-2-methoxyphenyl)amino]-2-(3-iodopyridin-4-yl)-1H,5H,6H,7H-pyrrolo[3,2-c]pyridin-4-one (300 mg, 0.61 mmol, 1.00 equiv) and tert-butyl 2-ethynylpiperidine-1-carboxylate (317 mg, 1.51 mmol, 2.50 equiv) in DMF (5 mL) were added Pd(dppf)Cl₂CH₂Cl₂ (124 mg, 0.15 mmol, 0.25 equiv) and CuI (57 mg, 0.30 mmol, 0.50 equiv) at room temperature. To the above mixture was added DIEA (235 mg, 1.82 mmol, 3.00 equiv) dropwise at room temperature. The resulting suspension was backfilled with argon three times and stirred for 2h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by reverse flash chromatography with the following conditions: column, silica gel; mobile phase, MeCN in water, 10% to 50% gradient in 10 min; detector, UV 254 nm. The resulting mixture was concentrated under reduced pressure. The residue was continue purified by silica gel column chromatography, eluted with CH₂Cl₂/MeOH (10:1) to afford tert-butyl2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]piperidine-1-carboxylate (227 mg, 64.98%) as a light yellow solid.

LC-MS: M+H found: 576.2.

### 135.2. Synthesis of 3-[(3-chloro-2-methoxyphenyl)amino]-2-{3-[2-(piperidin-2-yl)ethynyl]pyridin-4-yl}-1H,5H,6H,7H-pyrrolo[3,2-c]pyridin-4-one

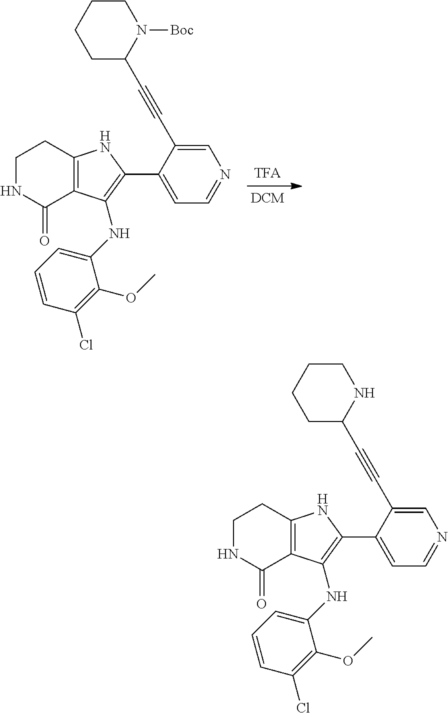 [SMILES: COc1c(Cl)cccc1Nc1c(-c2ccncc2C#CC2CCCCN2C(=O)OC(C)(C)C)[nH]c2c1C(=O)NCC2 ; COc1c(Cl)cccc1Nc1c(-c2ccncc2C#CC2CCCCN2)[nH]c2c1C(=O)NCC2]

To a stirred mixture of tert-butyl 2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]piperidine-1-carboxylate (185 mg, 0.32 mmol, 1.00 equiv) in DCM (3 mL) was added TFA (1 mL) at room temperature under nitrogen atmosphere. The resulting mixture was stirred for 1 h and concentrated under reduced pressure to afford 3-[(3-chloro-2-methoxyphenyl)amino]-2-{3-[2-(piperidin-2-yl)ethynyl]pyridin-4-yl}-1H,5H,6H,7H-pyrrolo[3,2-c]pyridin-4-one (230 mg, crude) as a red oil.

LC-MS: (M+H)⁺ found 476.2.

### 135.3. Synthesis of 3-[(3-chloro-2-methoxyphenyl)amino]-2-(3-{2-[1-(prop-2-enoyl)piperidin-2-yl]ethynyl}pyridin-4-yl)-1H,5H,6H,7H-pyrrolo[3,2-c]pyridin-4-one

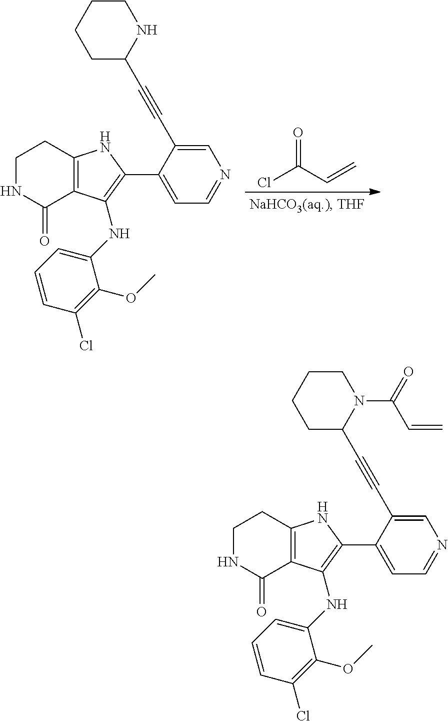

Into a 50 mL round-bottom flask were added 3-[(3-chloro-2-methoxyphenyl)amino]-2-{3-[2-(piperidin-2-yl)ethynyl]pyridin-4-yl}-1H,5H,6H,7H-pyrrolo[3,2-c]pyridin-4-one (153 mg, 0.32 mmol, 1.00 equiv) and THF (6 mL) at room temperature. The mixture was basified to pH 8 with saturated NaHCO₃(aq.). To the above mixture was added acryloyl chloride (38 mg, 0.41 mmol, 1.30 equiv) dropwise at 0° C. The resulting mixture was stirred for additional 2h at room temperature. The reaction was monitored by LCMS. Desired product could be detected by LCMS. The resulting mixture was extracted with CH₂Cl₂:MeOH(10:1)(3×40 mL), dried over anhydrous Na₂SO₄. After filtration, the filtrate was concentrated under reduced pressure. The resulting mixture was concentrated under reduced pressure. The residue was purified by silica gel column chromatography, eluted with CH₂Cl₂/MeOH (10:1) to afford 3-[(3-chloro-2-methoxyphenyl)amino]-2-(3-{2-[1-(prop-2-enoyl)piperidin-2-yl]ethynyl}pyridin-4-yl)-1H,5H,6H,7H-pyrrolo[3,2-c]pyridin-4-one (60 mg, 37.01%) as a red solid.

LC-MS: (M+H)⁺ found 530.15.

### 135.4. Synthesis of 3-[(3-chloro-2-methoxyphenyl)amino]-2-(3-{2-[(2R)-1-(prop-2-enoyl)piperidin-2-yl]ethynyl}pyridin-4-yl)-1H,5H,6H,7H-pyrrolo[3,2-c]pyridin-4-one

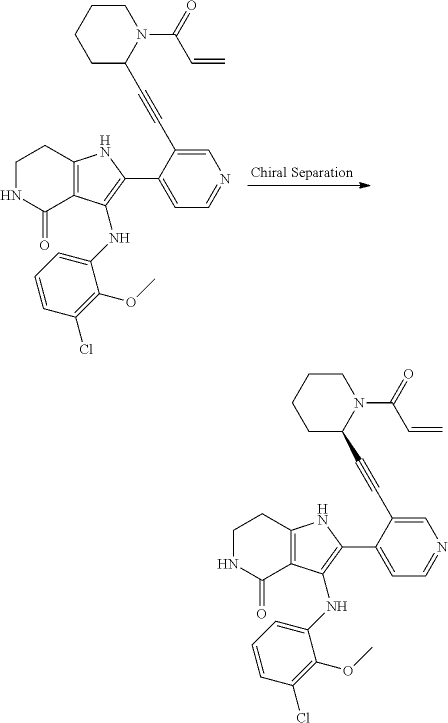 [SMILES: C=CC(=O)N1CCCCC1C#Cc1cnccc1-c1[nH]c2c(c1Nc1cccc(Cl)c1OC)C(=O)NCC2 ; C=CC(=O)N1CCCC[C@@H]1C#Cc1cnccc1-c1[nH]c2c(c1Nc1cccc(Cl)c1OC)C(=O)NCC2]

The crude product (60 mg) was purified by Prep-CHIRAL-HPLC with the following conditions (Column: CHIRALPAK IH-3, 4.6*50 mm, 3 m; Mobile Phase A: Hex (0.1% DEA):EtOH=60:40; Flow rate: 1 mL/min; Gradient: 0% B to 0% B; Injection Volume: 5 ul mL) to afford 3-[(3-chloro-2-methoxyphenyl)amino]-2-(3-{2-[(2R)-1-(prop-2-enoyl)piperidin-2-yl]ethynyl}pyridin-4-yl)-1H,5H,6H,7H-pyrrolo[3,2-c]pyridin-4-one (4.5 mg, 6.52%) as a light yellow solid.

LC-MS: (M+H)⁺ found 530.4.

¹H-NMR (400 MHz, Chloroform-d) δ 10.54 (s, 1H), 8.60 (s, 1H), 8.11 (d, 1H), 7.94 (s, 1H), 7.36 (d, 1H), 6.78 (d, 1H), 6.69-6.53 (m, 2H), 6.36-6.29 (m, 1H), 6.22-6.13 (m, 1H), 5.82-5.76 (m, 1H), 5.63 (s, 1H), 5.30 (s, 1H), 4.09 (s, 3H), 3.95-3.87 (s, 1H), 3.71-3.56 (m, 2H), 3.42-3.30 (m, 1H), 3.29-3.12 (m, 2H), 2.11-2.05 (m, 1H), 1.90 (d, 5H).

### Example 136. 3-[(3-chloro-2-methoxyphenyl)amino]-2-(3-{2-[(2S)-1-(prop-2-enoyl)piperidin-2-yl]ethynyl}pyridin-4-yl)-1H,5H,6H,7H-pyrrolo[3,2-c]pyridin-4-one (compound 230)

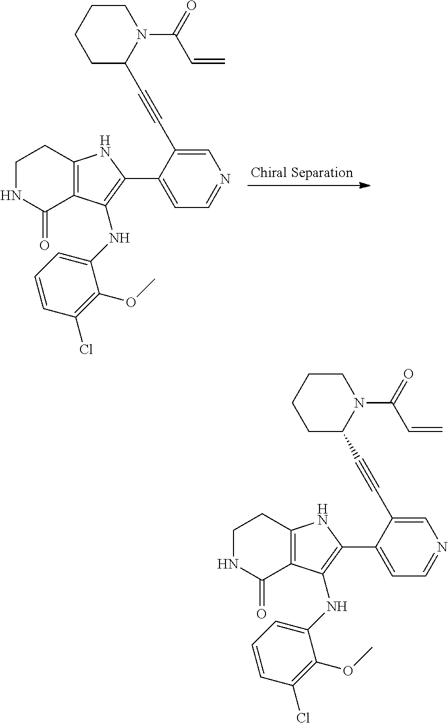 [SMILES: C=CC(=O)N1CCCCC1C#Cc1cnccc1-c1[nH]c2c(c1Nc1cccc(Cl)c1OC)C(=O)NCC2 ; C=CC(=O)N1CCCC[C@H]1C#Cc1cnccc1-c1[nH]c2c(c1Nc1cccc(Cl)c1OC)C(=O)NCC2]

The crude product (69 mg) was purified by Prep-CHIRAL-HPLC with the following conditions (Column: CHIRALPAK IH-3, 4.6*50 mm, 3 m; Mobile Phase A: Hex (0.1% DEA):EtOH=60:40; Flow rate: 1 mL/min; Gradient: 0% B to 0% B; Injection Volume: 5 ul mL) to afford 3-[(3-chloro-2-methoxyphenyl)amino]-2-(3-{2-[(2S)-1-(prop-2-enoyl)piperidin-2-yl]ethynyl}pyridin-4-yl)-1H,5H,6H,7H-pyrrolo[3,2-c]pyridin-4-one (6.8 mg, 10.37%) as a light yellow solid.

LC-MS: (M+H)⁺ found 530.40.

¹H NMR (400 MHz, Chloroform-d) δ 10.49 (s, 1H), 8.60 (s, 1H), 8.13 (d, 1H), 7.87 (s, 1H), 7.37 (d, 1H), 6.78 (d, 1H), 6.68-6.58 (m, 2H), 6.34-6.28 (m, 1H), 6.21-6.17 (m, 1H), 5.80-5.73 (m, 1H), 5.66 (s, 1H), 5.29 (s, 1H), 4.09 (s, 3H), 3.99-3.89 (m, 1H), 3.69-3.55 (m, 2H), 3.42-3.09 (m, 3H), 2.12-2.02 (m, 1H), 1.92 (d, 5H).

### Example 137. 2-(3-{2-[(2R)-4,4-dimethyl-1-(prop-2-enoyl)pyrrolidin-2-yl]ethynyl}pyridin-4-yl)-3-[(3-fluoro-2-methoxyphenyl)amino]-1H,5H,6H,7H-pyrrolo[3,2-c]pyridin-4-one (compound 253)

### 137.1. Synthesis of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-4,4-dimethylpyrrolidine-1-carboxylate

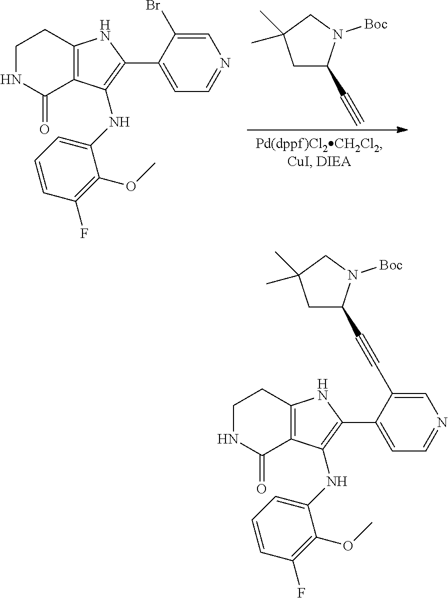

To a stirred mixture of 2-(3-bromopyridin-4-yl)-3-[(3-fluoro-2-methoxyphenyl)amino]-1H,5H,6H,7H-pyrrolo[3,2-c]pyridin-4-one (200 mg, 0.46 mmol, 1.00 equiv) and CuI (44 mg, 0.23 mmol, 0.50 equiv) and Pd(dppf)Cl₂CH₂Cl₂ (94 mg, 0.11 mmol, 0.25 equiv) in DMF (4 mL) were added tert-butyl (2R)-2-ethynyl-4,4-dimethylpyrrolidine-1-carboxylate (207 mg, 0.92 mmol, 2.00 equiv) and DIEA (299 mg, 2.32 mmol, 5.00 equiv) dropwise at room temperature under argon atmosphere. The resulting suspension was backfilled with argon three times and stirred for 2h at 50° C. LCMS confirmed completion of reaction and desired product was observed. The resulting mixture was concentrated under vacuum. The residue was purified by Prep-HPLC with the following conditions (0.1% NH₄HCO₃ in water and ACN (33% ACN up to 90% in 20 min) to afford tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-4,4-dimethylpyrrolidine-1-carboxylate (180 mg, 66.9%) as a yellow solid.

LC-MS:(M+H)⁺ found: 574.15.

### 137.2. Synthesis of 2-(3-{2-[(2R)-4,4-dimethylpyrrolidin-2-yl] ethynyl} pyridin-4-yl)-3-[(3-fluoro-2-methoxyphenyl) amino]-1H,5H,6H,7H-pyrrolo[3,2-c] pyridin-4-one

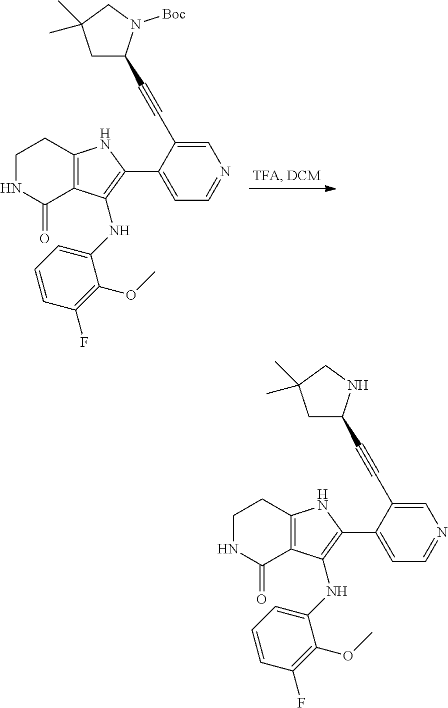 [SMILES: COc1c(F)cccc1Nc1c(-c2ccncc2C#C[C@H]2CC(C)(C)CN2C(=O)OC(C)(C)C)[nH]c2c1C(=O)NCC2 ; COc1c(F)cccc1Nc1c(-c2ccncc2C#C[C@H]2CC(C)(C)CN2)[nH]c2c1C(=O)NCC2]

To a stirred mixture of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-4,4-dimethylpyrrolidine-1-carboxylate (80 mg, 0.13 mmol, 1.00 equiv) in DCM (3 mL) was added TFA (1 mL) at room temperature under nitrogen atmosphere. The resulting mixture was stirred for 1 h and concentrated under reduced pressure to afford 2-(3-{2-[(2R)-4,4-dimethylpyrrolidin-2-yl] ethynyl} pyridin-4-yl)-3-[(3-fluoro-2-methoxyphenyl) amino]-1H,5H,6H,7H-pyrrolo[3,2-c] pyridin-4-one (110 mg, crude) as a red oil.

LC-MS: (M+H)⁺ found: 474.25.

### 137.3. Synthesis of 2-(3-{2-[(2R)-4,4-dimethyl-1-(prop-2-enoyl)pyrrolidin-2-yl]ethynyl}pyridin-4-yl)-3-[(3-fluoro-2-methoxyphenyl)amino]-1H,5H,6H,7H-pyrrolo[3,2-c]pyridin-4-one

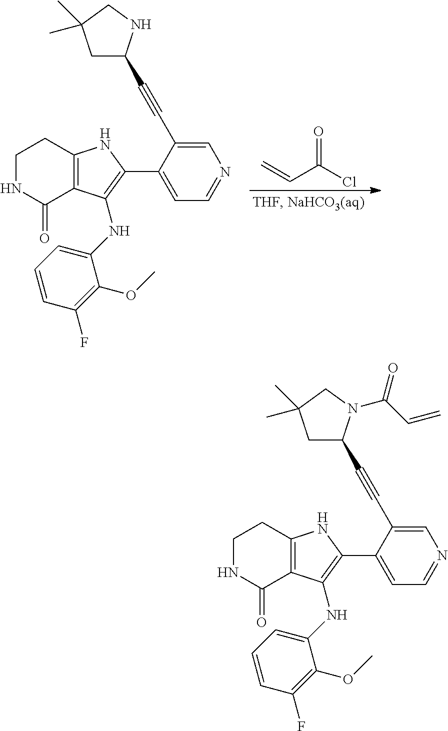

To a stirred solution of 2-(3-{2-[(2R)-4,4-dimethylpyrrolidin-2-yl] ethynyl} pyridin-4-yl)-3-[(3-fluoro-2-methoxyphenyl) amino]-1H,5H,6H,7H-pyrrolo[3,2-c] pyridin-4-one (80 mg, 0.17 mmol, 1.00 equiv) and sat. NaHCO₃(aq.) (3.00 mL) in THF (3.00 mL) was added acryloyl chloride (15 mg, 0.17 mmol, 1.00 equiv) dropwise at 0° C. under nitrogen atmosphere. The resulting mixture was stirred for 1 h at room temperature under nitrogen atmosphere. The resulting mixture was extracted with CH₂Cl₂/MeOH (10/1) (3×30 mL). The combined organic layers were washed with brine (3×30 mL), dried over anhydrous Na₂SO₄. After filtration, the filtrate was concentrated under reduced pressure. The residue was purified by Prep-HPLC with the following conditions: (Column: Xselect CSH C18 OBD Column 30*150 mm 5 m, n; Mobile Phase A: Water (0.1% FA), Mobile Phase B: ACN; Flow rate: 60 mL/min; Gradient: 32% B to 50% B in 8 min, 50% B; Wave Length: 254/220 nm; RT1(min): 8; Number Of Runs: 0) to afford 2-(3-{2-[(2R)-4,4-dimethyl-1-(prop-2-enoyl)pyrrolidin-2-yl]ethynyl}pyridin-4-yl)-3-[(3-fluoro-2-methoxyphenyl)amino]-1H,5H,6H,7H-pyrrolo[3,2-c]pyridin-4-one (38.5 mg, 42.3%) as a yellow solid.

LC-MS: (M+H)⁺ found 528.50.

¹H-NMR (400 MHz, Chloroform-d) δ 11.14 (s, 1H), 8.47 (s, 1H), 8.07 (s, 1H), 7.67 (s, 1H), 7.43 (s, 1H), 6.56-6.27 (m, 4H), 6.02 (d, 1H), 5.71-5.67 (m, 1H), 5.24 (s, 1H), 4.87 (t, 1H), 4.03 (d, 3H), 3.60-3.43 (m, 3H), 3.39-3.32 (m, 1H), 3.25-3.14 (m, 2H), 2.19-2.11 (m, 1H), 2.06-1.98 (m, 1H), 1.27 (s, 3H), 1.08 (s, 3H).

### Example 138. (2E)-4-(dimethylamino)-N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]but-2-enamide (compound 334)

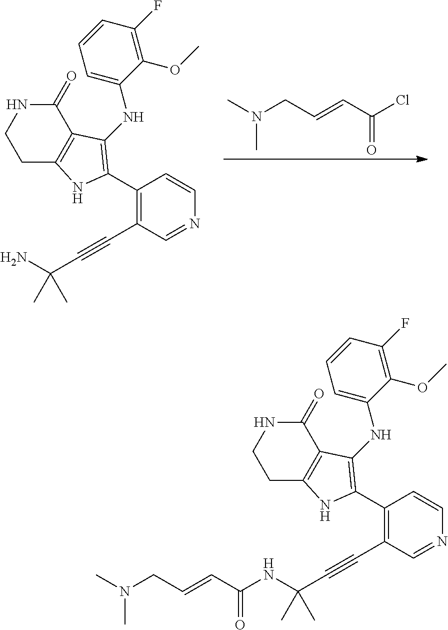

To a stirred solution of 2-[3-(3-amino-3-methylbut-1-yn-1-yl)pyridin-4-yl]-3-[(3-fluoro-2-methoxyphenyl)amino]-1H,5H,6H,7H-pyrrolo[3,2-c]pyridin-4-one (80 mg, 0.18 mmol, 1.00 equiv) and DIEA (119 mg, 0.93 mmol, 5.00 equiv) in THF (2.00 mL) was added (2E)-4-(dimethylamino)but-2-enoyl chloride (29 mg, 0.20 mmol, 1.10 equiv) dropwise at 0° C. under argon atmosphere. The resulting mixture was stirred for 1 h at 0° C. under argon atmosphere. The reaction was quenched with water at 0° C. The resulting mixture was extracted with EtOAc (3×5 mL). The combined organic layers were washed with brine (1×10 mL), dried over anhydrous Na₂SO₄. After filtration, the filtrate was concentrated under reduced pressure. The crude product was purified by Prep-HPLC with the following conditions (Column: XSelect CSH Fluoro Phenyl, 30*150 mm, 5 μm; Mobile Phase A: Water (10 mmol/L NH₄HCO₃), Mobile Phase B: ACN; Flow rate: 60 mL/min; Gradient: 43% B to 73% B in 7 min, 73% B; Wave Length: 254 nm; RT1(min): 6.55; Number Of Runs: 0) to afford (2E)-4-(dimethylamino)-N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]but-2-enamide (30.4 mg, 30.09%) as an off-white solid.

LC-MS: (M+H)⁺ found: 545.20

¹H NMR (400 MHz, CDCl3) δ 11.40 (s, 1H), 8.53 (s, 1H), 8.15 (d, 1H), 7.45 (d, 1H), 6.90-6.80 (m, 1H), 6.63-6.55 (m, 1H), 6.50-6.42 (m, 1H), 6.14-5.98 (m, 2H), 5.23 (s, 1H), 4.09 (d, 3H), 3.65-3.58 (m, 2H), 3.25-3.12 (m, 4H), 2.35 (s, 6H), 1.73 (s, 6H).

### Example 139. 3-[(3-fluoro-2-methoxyphenyl)amino]-2-(3-{2-[(6R)-5-(prop-2-enoyl)-5-azaspiro[2.4]heptan-6-yl]ethynyl}pyridin-4-yl)-1H,5H,6H,7H-pyrrolo[3,2-c]pyridin-4-one (compound 251)

### 139.1. Synthesis of tert-butyl (6R)-6-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-azaspiro[2.4]heptane-5-carboxylate

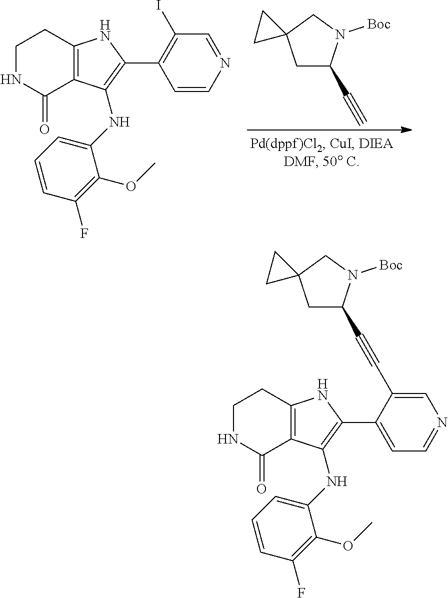

To a stirred mixture of 3-[(3-fluoro-2-methoxyphenyl)amino]-2-(3-iodopyridin-4-yl)-1H,5H,6H,7H-pyrrolo[3,2-c]pyridin-4-one (80 mg, 0.16 mmol, 1.00 equiv) and tert-butyl (6R)-6-ethynyl-5-azaspiro[2.4]heptane-5-carboxylate (46 mg, 0.21 mmol, 5.00 equiv) in dimethylformamide (1.00 mL) were added copper(I) iodide (15 mg, 0.08 mmol, 0.50 equiv), Pd(dppf)C12DCM (34 mg, 0.04 mmol, 0.25 equiv) and DIEA (108 mg, 0.83 mmol, 5.00 equiv) at room temperature under argon atmosphere. The resulting suspension was backfilled with argon three times and stirred for 2 h at 50° C. LCMS confirmed completion of reaction and desired product was observed. The resulting mixture was purified by reverse flash chromatography with the following conditions: column, C18 gel; mobile phase, MeCN in water, 10% to 50% gradient in 10 min; detector, UV 254 nm. This resulted in tert-butyl (6R)-6-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-azaspiro[2.4]heptane-5-carboxylate (80 mg, 83.66%) as a yellow solid.

LC-MS: (M+H)⁺ found 572.30.

### 139.2. Synthesis of 2-(3-{2-[(6R)-5-azaspiro[2.4]heptan-6-yl]ethynyl}pyridin-4-yl)-3-[(3-fluoro-2-methoxyphenyl)amino]-1H,5H,6H,7H-pyrrolo[3,2-c]pyridin-4-one

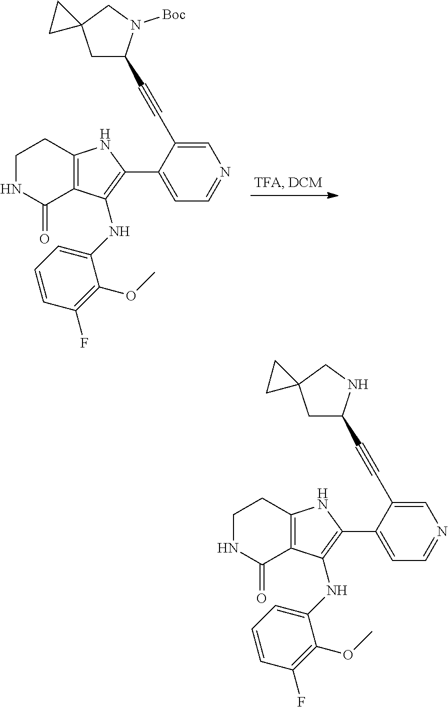 [SMILES: COc1c(F)cccc1Nc1c(-c2ccncc2C#C[C@H]2CC3(CC3)CN2C(=O)OC(C)(C)C)[nH]c2c1C(=O)NCC2 ; COc1c(F)cccc1Nc1c(-c2ccncc2C#C[C@H]2CC3(CC3)CN2)[nH]c2c1C(=O)NCC2]

To a stirred solution of tert-butyl (6R)-6-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-5-azaspiro[2.4]heptane-5-carboxylate (70 mg, 0.12 mmol, 1.00 equiv) in DCM (6.00 mL) was added TFA (2.00 mL) dropwise at 0° C. The resulting mixture was stirred for 2 h at room temperature and dried using nitrogen to afford 2-(3-{2-[(6R)-5-azaspiro[2.4]heptan-6-yl]ethynyl}pyridin-4-yl)-3-[(3-fluoro-2-methoxyphenyl)amino]-1H,5H,6H,7H-pyrrolo[3,2-c]pyridin-4-one (57 mg, crude) as a red oil.

LC-MS: (M+H)⁺ found 472.10.

### 139.3. Synthesis of 3-[(3-fluoro-2-methoxyphenyl)amino]-2-(3-{2-[(6R)-5-(prop-2-enoyl)-5-azaspiro[2.4]heptan-6-yl]ethynyl}pyridin-4-yl)-1H,5H,6H,7H-pyrrolo[3,2-c]pyridin-4-one

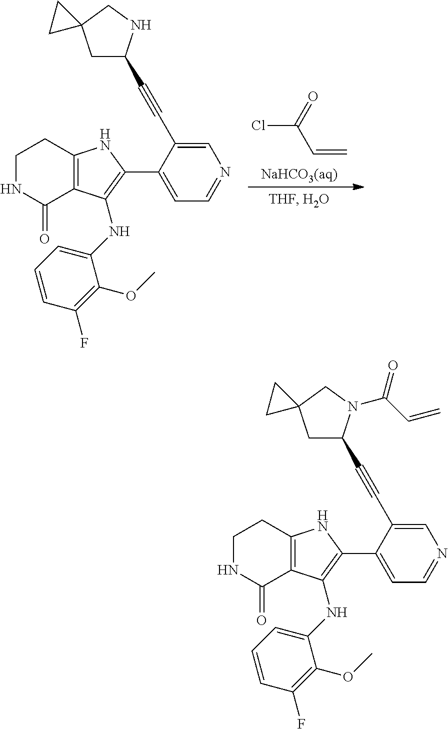

A solution of 2-(3-{2-[(6R)-5-azaspiro[2.4]heptan-6-yl]ethynyl}pyridin-4-yl)-3-[(3-fluoro-2-methoxyphenyl)amino]-1H,5H,6H,7H-pyrrolo[3,2-c]pyridin-4-one (57 mg, 0.12 mmol, 1.00 equiv) in THF (0.40 mL) was basified to pH 8 with NaHCO₃(aq.) (0.40 mL). To the above mixture was added acryloyl chloride (12 mg, 0.13 mmol, 1.10 equiv) at 0° C. under nitrogen atmosphere dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product that was purified by Prep-HPLC with the following conditions (Column: Xselect CSH F-Phenyl OBD column, 19*250 mm, 5 μm; Mobile Phase A: Water (0.1% FA), Mobile Phase B: MeOH-Preparative; Flow rate: 25 mL/min; Gradient: 47% B to 77% B in 7 min, 77% B; Wave Length: 254 nm; RT1(min): 6.67; Number Of Runs: 0) to afford 3-[(3-fluoro-2-methoxyphenyl)amino]-2-(3-{2-[(6R)-5-(prop-2-enoyl)-5-azaspiro[2.4]heptan-6-yl]ethynyl}pyridin-4-yl)-1H,5H,6H,7H-pyrrolo[3,2-c]pyridin-4-one (7.0 mg, 11.01%) as a yellow solid.

LC-MS: (M+H)⁺ found 526.45.

¹H NMR (400 MHz, Chloroform-d) δ 11.25 (s, 1H), 8.51 (s, 1H), 8.06-7.99 (m, 2H), 7.46 (d, 1H), 6.59-6.51 (m, 2H), 6.41-6.39 (m, 2H), 6.07-6.05 (m, 1H), 5.79-5.76 (m, 1H), 5.26 (s, 1H), 5.03-5.00 (m, 1H), 4.10 (d, 3H), 3.67-3.56 (m, 4H), 3.33-3.29 (m, 2H), 2.34-2.29 (m, 1H), 2.18-2.14 (m, 1H), 0.90-0.80 (m, 2H), 0.74-0.68 (m, 2H).

### Example 140. rel-2-(3-{2-[(2R)-3,3-dimethyl-1-(prop-2-enoyl)azetidin-2-yl]ethynyl}pyridin-4-yl)-3-[(3-fluoro-2-methoxyphenyl)amino]-1H,5H,6H,7H-pyrrolo[3,2-c]pyridin-4-one (compound 250)

### 140.1. Synthesis of tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-3,3-dimethylazetidine-1-carboxylate

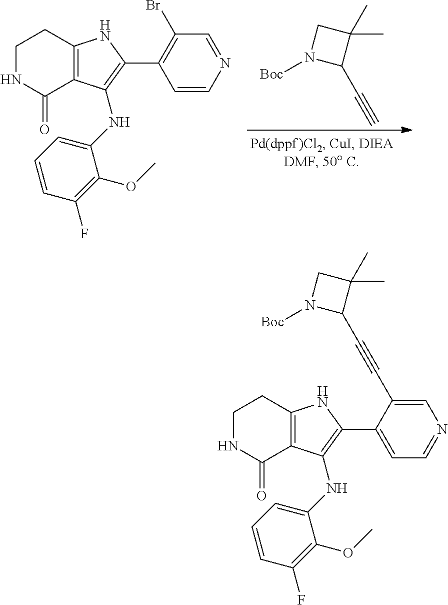 [SMILES: COc1c(F)cccc1Nc1c(-c2ccncc2Br)[nH]c2c1C(=O)NCC2 ; C#CC1N(C(=O)OC(C)(C)C)CC1(C)C ; COc1c(F)cccc1Nc1c(-c2ccncc2C#CC2N(C(=O)OC(C)(C)C)CC2(C)C)[nH]c2c1C(=O)NCC2]

To a stirred solution of 2-(3-bromopyridin-4-yl)-3-[(3-fluoro-2-methoxyphenyl)amino]-1H,5H,6H,7H-pyrrolo[3,2-c]pyridin-4-one (350 mg, 0.81 mmol, 1.00 equiv) and Pd(dppf)C12-CH₂Cl₂ (165 mg, 0.20 mmol, 0.25 equiv) and CuI (77 mg, 0.40 mmol, 0.50 equiv) in DMF (3.00 mL) were added tert-butyl 2-ethynyl-3,3-dimethylazetidine-1-carboxylate (509 mg, 2.43 mmol, 3.00 equiv) and DIEA (524 mg, 4.06 mmol, 5.00 equiv) at room temperature under Ar atmosphere. The resulting suspension was backfilled with argon three times and stirred for 2 h at 50° C. LCMS confirmed completion of reaction and desired product was observed. The resulting mixture was purified by reverse flash chromatography with the following conditions (column, C18 gel; mobile phase, MeCN in water, 10% to 80% gradient in 30 min; detector, UV 254 nm) to afford tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-3,3-dimethylazetidine-1-carboxylate (310 mg) as a yellow solid. The residue was purified by silica gel column chromatography, eluted with CH₂Cl₂/MeOH (10:1) to afford tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-3,3-dimethylazetidine-1-carboxylate (300 mg, 66.05%) as a yellow solid.

LC-MS: (M+H)⁺ found: 560.2.

### 140.2. Synthesis of 2-{3-[2-(3,3-dimethylazetidin-2-yl)ethynyl]pyridin-4-yl}-3-[(3-fluoro-2-methoxyphenyl)amino]-1H,5H,6H,7H-pyrrolo[3,2-c]pyridin-4-one

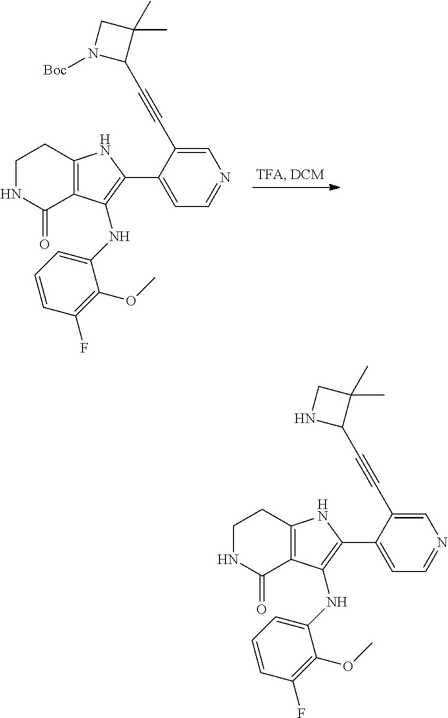 [SMILES: COc1c(F)cccc1Nc1c(-c2ccncc2C#CC2N(C(=O)OC(C)(C)C)CC2(C)C)[nH]c2c1C(=O)NCC2 ; COc1c(F)cccc1Nc1c(-c2ccncc2C#CC2NCC2(C)C)[nH]c2c1C(=O)NCC2]

To a stirred solution of tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-3,3-dimethylazetidine-1-carboxylate (180 mg, 0.32 mmol, 1.00 equiv) in DCM (4.00 mL) was added TFA (2.00 mL) dropwise at 0° C. The resulting mixture was stirred for 2 h at room temperature and dried using nitrogen to afford 2-{3-[2-(3,3-dimethylazetidin-2-yl)ethynyl]pyridin-4-yl}-3-[(3-fluoro-2-methoxyphenyl)amino]-1H,5H,6H,7H-pyrrolo[3,2-c]pyridin-4-one (160 mg, crude) as a red oil.

LC-MS: (M+H)⁺ found: 460.2.

### 140.3. Synthesis of 2-(3-{2-[3,3-dimethyl-1-(prop-2-enoyl)azetidin-2-yl]ethynyl}pyridin-4-yl)-3-[(3-fluoro-2-methoxyphenyl)amino]-1H,5H,6H,7H-pyrrolo[3,2-c]pyridin-4-one

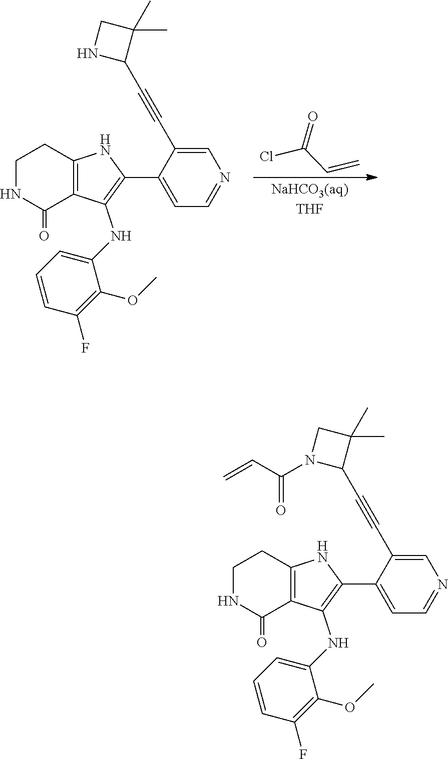

A solution of 2-{3-[2-(3,3-dimethylazetidin-2-yl)ethynyl]pyridin-4-yl}-3-[(3-fluoro-2-methoxyphenyl)amino]-1H,5H,6H,7H-pyrrolo[3,2-c]pyridin-4-one (160 mg, 0.34 mmol, 1.00 equiv) in THF (1.50 mL) was basified to pH 8 with NaHCO₃aq (1.5 mL). To the above mixture was added acryloyl chloride (37 mg, 0.41 mmol, 1.20 equiv) at 0° C. under nitrogen atmosphere dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product that was purified by Prep-HPLC under the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 26% B to 56% B in 7 min; Wave Length: 254 nm; RT1(min): 6.5; Number Of Runs: 0) to afford 2-(3-{2-[3,3-dimethyl-1-(prop-2-enoyl)azetidin-2-yl]ethynyl}pyridin-4-yl)-3-[(3-fluoro-2-methoxyphenyl)amino]-1H,5H,6H,7H-pyrrolo[3,2-c]pyridin-4-one (90 mg, 50.33%) as a yellow solid.

LC-MS: (M+H)⁺ found: 514.20.

### 140.4. Synthesis of rel-2-(3-{2-[(2R)-3,3-dimethyl-1-(prop-2-enoyl)azetidin-2-yl]ethynyl}pyridin-4-yl)-3-[(3-fluoro-2-methoxyphenyl)amino]-1H,5H,6H,7H-pyrrolo[3,2-c]pyridin-4-one

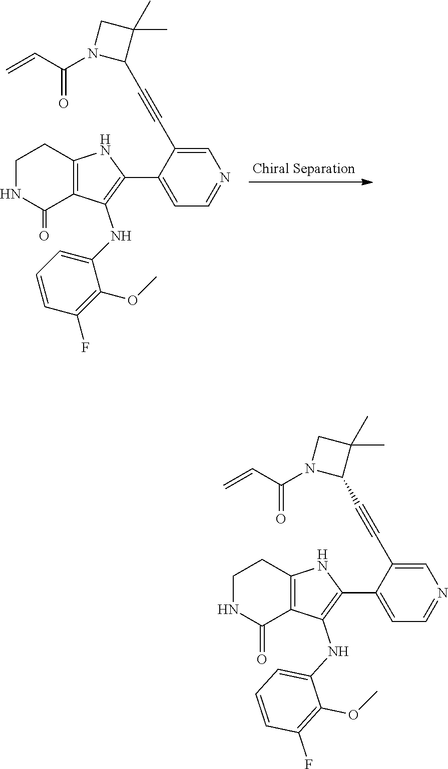 [SMILES: C=CC(=O)N1CC(C)(C)C1C#Cc1cnccc1-c1[nH]c2c(c1Nc1cccc(F)c1OC)C(=O)NCC2 ; C=CC(=O)N1CC(C)(C)[C@@H]1C#Cc1cnccc1-c1[nH]c2c(c1Nc1cccc(F)c1OC)C(=O)NCC2]

2-(3-f{2-[3,3-dimethyl-1-(prop-2-enoyl)azetidin-2-yl]ethynyl}pyridin-4-yl)-3-[(3-fluoro-2-methoxyphenyl)amino]-1H,5H,6H,7H-pyrrolo[3,2-c]pyridin-4-one (90 mg) was separated by Chiral-HPLC with the following conditions (Column: CHIRALPAK IG, 2*25 cm, 5 μm; Mobile Phase A: Hex:DCM=3:1(0.5% 2M NH₃-MeOH)—HPLC, Mobile Phase B: EtOH—HPLC; Flow rate: 20 mL/min; Gradient: 10% B to 10% B in 22 min; Wave Length: 220/254 nm; RT1(min): 17.99; RT2(min): 21.96; Sample Solvent: ETOH:DCM=1:1; Injection Volume: 1 mL; Number Of Runs: 3) to afford rel-2-(3-{2-[(2R)-3,3-dimethyl-1-(prop-2-enoyl)azetidin-2-yl]ethynyl}pyridin-4-yl)-3-[(3-fluoro-2-methoxyphenyl)amino]-1H,5H,6H,7H-pyrrolo[3,2-c]pyridin-4-one (31.0 mg, 34.44%) as a yellow solid.

LC-MS: (M+H)⁺ found: 514.10.

¹H NMR (300 MHz, Chloroform-d) δ 10.94 (s, 1H), 8.56 (s, 1H), 8.13 (d, 1H), 7.83 (s, 1H), 7.46 (d, 1H), 6.61-6.45 (m, 2H), 6.40-6.34 (m, 1H), 6.25-6.16 (m, 1H), 6.08 (d, 1H), 5.78-5.74 (m, 1H), 5.21 (s, 1H), 4.86 (s, 1H), 4.10 (s, 3H), 3.97 (s, 2H), 3.59 (d, 2H), 3.24 (t, 2H), 1.54 (s, 3H), 1.45 (s, 3H).

### Example 141. rel-2-(3-{2-[(2R)-3,3-dimethyl-1-(prop-2-enoyl)azetidin-2-yl]ethynyl}pyridin-4-yl)-3-[(3-fluoro-2-methoxyphenyl)amino]-1H,5H,6H,7H-pyrrolo[3,2-c]pyridin-4-one (compound 249)

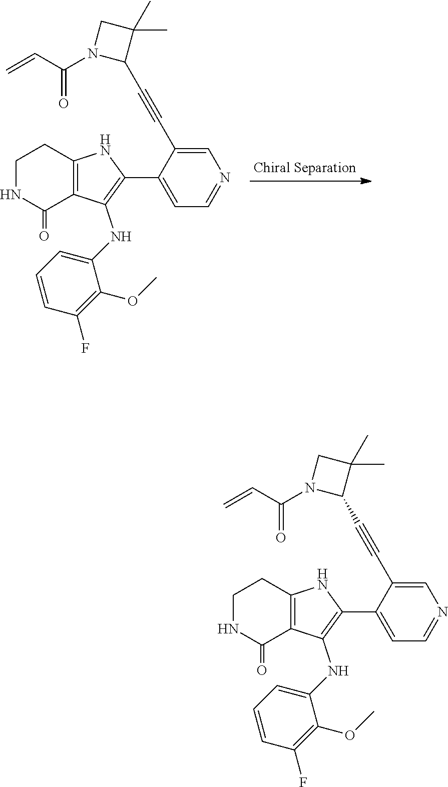 [SMILES: C=CC(=O)N1CC(C)(C)C1C#Cc1cnccc1-c1[nH]c2c(c1Nc1cccc(F)c1OC)C(=O)NCC2 ; C=CC(=O)N1CC(C)(C)[C@@H]1C#Cc1cnccc1-c1[nH]c2c(c1Nc1cccc(F)c1OC)C(=O)NCC2]

2-(3-{2-[3,3-dimethyl-1-(prop-2-enoyl)azetidin-2-yl]ethynyl}pyridin-4-yl)-3-[(3-fluoro-2-methoxyphenyl)amino]-1H,5H,6H,7H-pyrrolo[3,2-c]pyridin-4-one (90 mg) was separated by Chiral-HPLC with the following conditions (Column: CHIRALPAK IG-3, 4.6*50 mm, 3 um; Mobile Phase A: (Hex:DCM=3:1)(0.1% DEA):EtOH=90:10; Flow rate: 1 mL/min; Gradient: 0% B to 0% B; Injection Volume: 5 ul mL) to afford rel-2-(3-{2-[(2R)-3,3-dimethyl-1-(prop-2-enoyl)azetidin-2-yl]ethynyl}pyridin-4-yl)-3-[(3-fluoro-2-methoxyphenyl)amino]-1H,5H,6H,7H-pyrrolo[3,2-c]pyridin-4-one (31.7 mg, 35.22%) as a yellow solid.

LC-MS: (M+H)⁺ found: 514.15.

¹H NMR (300 MHz, Chloroform-d) δ 10.94 (s, 1H), 8.56 (s, 1H), 8.13 (d, 1H), 7.83 (s, 1H), 7.46 (d, 1H), 6.61-6.45 (m, 2H), 6.40-6.34 (m, 1H), 6.25-6.16 (m, 1H), 6.08 (d, 1H), 5.78-5.74 (m, 1H), 5.23 (s, 1H), 4.86 (s, 1H), 4.10 (s, 3H), 3.97 (s, 2H), 3.59 (d, 2H), 3.24 (t, 2H), 1.54 (s, 3H), 1.45 (s, 3H).

### Example 142. 3-[(3-fluoro-2-methoxyphenyl)amino]-2-(3-{2-[(2R)-1-(prop-2-enoyl)piperidin-2-yl]ethynyl}pyridin-4-yl)-1H,5H,6H,7H-pyrrolo[3,2-c]pyridin-4-one (compound 248)

### 142.1. Synthesis of tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]piperidine-1-carboxylate

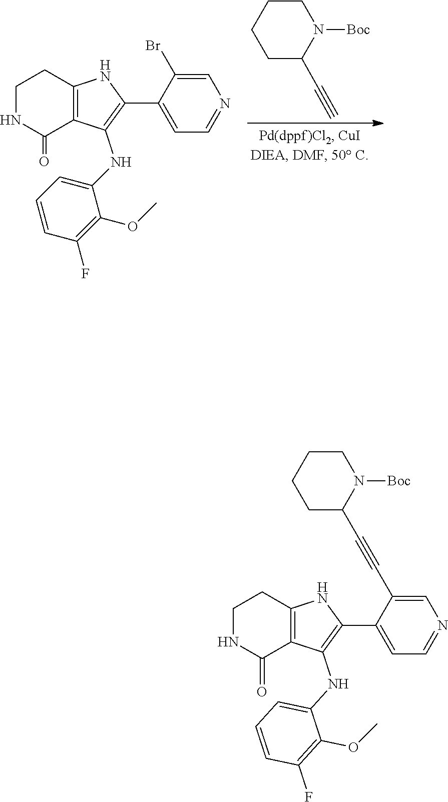 [SMILES: COc1c(F)cccc1Nc1c(-c2ccncc2Br)[nH]c2c1C(=O)NCC2 ; C#CC1CCCCN1C(=O)OC(C)(C)C ; COc1c(F)cccc1Nc1c(-c2ccncc2C#CC2CCCCN2C(=O)OC(C)(C)C)[nH]c2c1C(=O)NCC2]

To a stirred mixture of 2-(3-bromopyridin-4-yl)-3-[(3-fluoro-2-methoxyphenyl)amino]-1H,5H,6H,7H-pyrrolo[3,2-c]pyridin-4-one (300 mg, 0.70 mmol, 1.00 equiv) in DMF (3.00 mL) were added tert-butyl 2-ethynylpiperidine-1-carboxylate (436 mg, 2.08 mmol, 3.00 equiv), Pd(dppf)C12 CH₂Cl₂ (141 mg, 0.17 mmol, 0.25 equiv), CuI (66 mg, 0.34 mmol, 0.50 equiv) and DIEA (449 mg, 3.48 mmol, 5.00 equiv) at room temperature under argon atmosphere. The resulting suspension was backfilled with argon three times and stirred for 2 h at 50° C. LCMS confirmed completion of reaction and desired product was observed. The resulting mixture was purified by reverse flash chromatography with the following conditions (column, C 18 gel; mobile phase, MeCN in water, 10% to 60% gradient in 10 min; detector, UV 254 nm) to afford tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]piperidine-1-carboxylate (220 mg, 56.51%) as a yellow solid.

LC-MS: (M+H)⁺ found: 560.20.

### 142.2. Synthesis of 3-[(3-fluoro-2-methoxyphenyl)amino]-2-{3-[2-(piperidin-2-yl)ethynyl]pyridin-4-yl}-1H,5H,6H,7H-pyrrolo[3,2-c]pyridin-4-one

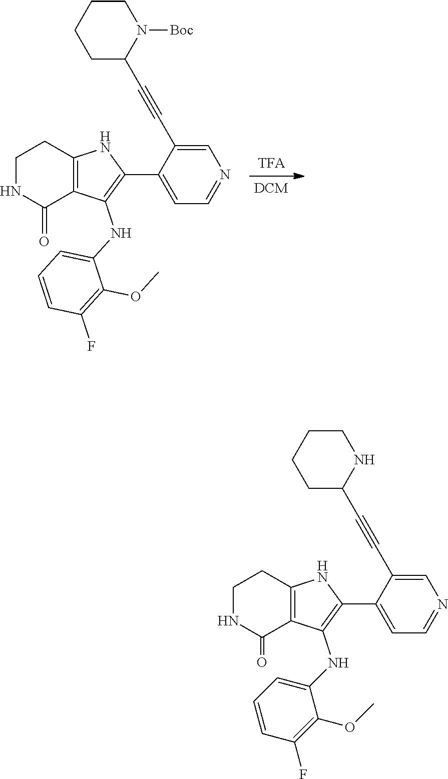 [SMILES: COc1c(F)cccc1Nc1c(-c2ccncc2C#CC2CCCCN2C(=O)OC(C)(C)C)[nH]c2c1C(=O)NCC2 ; COc1c(F)cccc1Nc1c(-c2ccncc2C#CC2CCCCN2)[nH]c2c1C(=O)NCC2]

To a stirred mixture of tert-butyl 2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]piperidine-1-carboxylate (200 mg, 0.36 mmol) in DCM (6.00 mL) was added TFA (2.00 mL) at room temperature under nitrogen atmosphere. The resulting mixture was stirred for 1 h and dried using nitrogen to afford 3-[(3-fluoro-2-methoxyphenyl)amino]-2-{3-[2-(piperidin-2-yl)ethynyl]pyridin-4-yl}-1H,5H,6H,7H-pyrrolo[3,2-c]pyridin-4-one (120 mg, crude) as a red oil.

LC-MS: (M+H)⁺ found: 460.10.

### 142.3. Synthesis of 3-[(3-fluoro-2-methoxyphenyl)amino]-2-(3-{2-[1-(prop-2-enoyl)piperidin-2-yl]ethynyl}pyridin-4-yl)-1H,5H,6H,7H-pyrrolo[3,2-c]pyridin-4-one

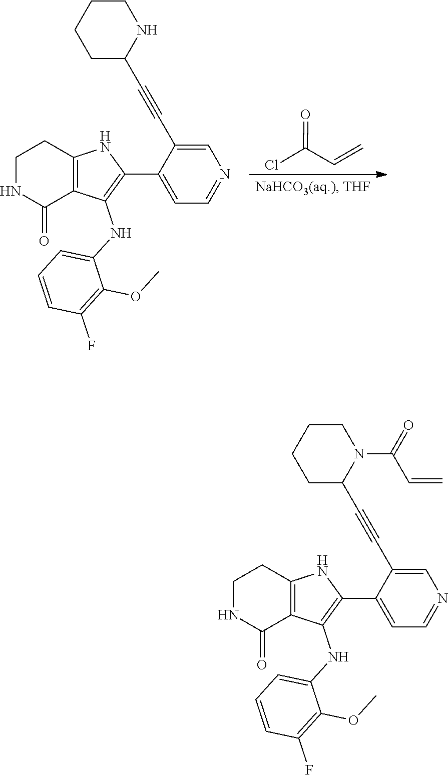

A solution of 3-[(3-fluoro-2-methoxyphenyl)amino]-2-{3-[2-(piperidin-2-yl)ethynyl]pyridin-4-yl}-1H,5H,6H,7H-pyrrolo[3,2-c]pyridin-4-one (120 mg, 0.26 mmol, 1.00 equiv) in THF (3.00 mL) was basified to pH 8 with NaHCO₃aq (3.00 mL). To the above mixture was added acryloyl chloride (23 mg, 0.26 mmol, 1.00 equiv) at 0° C. under nitrogen atmosphere dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product that was purified by silica gel column chromatography, eluted with CH₂Cl₂/MeOH (20:1) to afford 3-[(3-fluoro-2-methoxyphenyl)amino]-2-(3-{2-[1-(prop-2-enoyl)piperidin-2-yl]ethynyl}pyridin-4-yl)-1H,5H,6H,7H-pyrrolo[3,2-c]pyridin-4-one (50 mg, 27.96%) as a yellow solid.

LC-MS: (M+H)⁺ found: 514.40.

### 142.4. Synthesis of 3-[(3-fluoro-2-methoxyphenyl)amino]-2-(3-{2-[(2R)-1-(prop-2-enoyl)piperidin-2-yl]ethynyl}pyridin-4-yl)-1H,5H,6H,7H-pyrrolo[3,2-c]pyridin-4-one

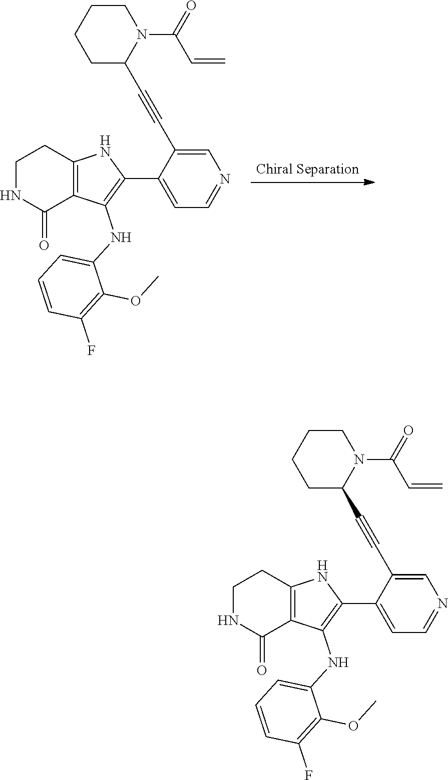 [SMILES: C=CC(=O)N1CCCCC1C#Cc1cnccc1-c1[nH]c2c(c1Nc1cccc(F)c1OC)C(=O)NCC2 ; C=CC(=O)N1CCCC[C@@H]1C#Cc1cnccc1-c1[nH]c2c(c1Nc1cccc(F)c1OC)C(=O)NCC2]

The racemic product (50 mg) was purified by Prep-CHIRAL-HPLC with the following conditions (Column: CHIRAL ART Cellulose-SB, 2*25 cm, 5 μm; Mobile Phase A: MtBE (0.5% 2M NH₃-MeOH)-HPLC, Mobile Phase B: MeOH:DCM=1:1(0.1% 2M NH₃-MEOH); Flow rate: 20 mL/min; Gradient: 10% B to 10% B in 21 min; Wave Length: 220/254 nm; RT1(min): 15.613; RT2(min): 17.482; Sample Solvent: EtOH—HPLC; Injection Volume: 0.3 mL; Number Of Runs: 12) to afford 3-[(3-fluoro-2-methoxyphenyl)amino]-2-(3-{2-[(2R)-1-(prop-2-enoyl)piperidin-2-yl]ethynyl}pyridin-4-yl)-1H,5H,6H,7H-pyrrolo[3,2-c]pyridin-4-one (10.2 mg, 20.32%) as a yellow solid.

LC-MS: (M+H)⁺ found: 514.10.

¹H NMR (400 MHz, Chloroform-d) δ 10.47 (s, 1H), 8.60 (s, 1H), 8.15 (d, 1H), 7.83 (s, 1H), 7.42 (d, 1H), 6.67-6.46 (m, 3H), 6.36-6.27 (m, 1H), 6.09-6.02 (m, 1H), 5.81-5.73 (m, 1H), 5.64 (s, 1H), 5.30 (s, 1H), 4.10 (d, 3H), 3.92 (s, 1H), 3.68-3.56 (m, 2H), 3.36 (s, 1H), 3.19 (s, 2H), 2.08 (s, 1H), 1.91-1.82 (m, 4H), 1.61 (s, 1H).

### Example 143. 3-[(3-fluoro-2-methoxyphenyl)amino]-2-(3-{2-[(2R)-1-(prop-2-enoyl)piperidin-2-yl]ethynyl}pyridin-4-yl)-1H,5H,6H,7H-pyrrolo[3,2-c]pyridin-4-one (compound 247)

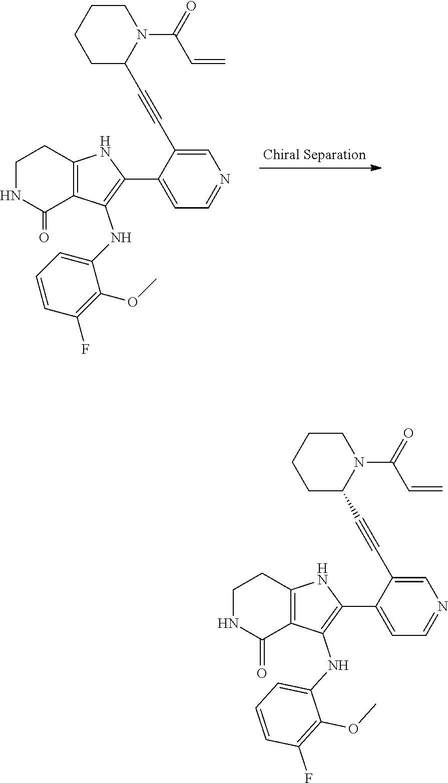 [SMILES: C=CC(=O)N1CCCCC1C#Cc1cnccc1-c1[nH]c2c(c1Nc1cccc(F)c1OC)C(=O)NCC2 ; C=CC(=O)N1CCCC[C@H]1C#Cc1cnccc1-c1[nH]c2c(c1Nc1cccc(F)c1OC)C(=O)NCC2]

The racemic product (50 mg) was purified by Prep-CHIRAL-HPLC with the following conditions (Column: CHIRAL ART Cellulose-SB, 2*25 cm, 5 μm; Mobile Phase A: MtBE (0.5% 2M NH₃-MeOH)—HPLC, Mobile Phase B: MeOH:DCM=1:1(0.1% 2M NH₃-MEOH); Flow rate: 20 mL/min; Gradient: 10% B to 10% B in 21 min; Wave Length: 220/254 nm; RT1(min): 15.613; RT2(min): 17.482; Sample Solvent: EtOH—HPLC; Injection Volume: 0.3 mL; Number Of Runs: 12) to afford 3-[(3-fluoro-2-methoxyphenyl)amino]-2-(3-{2-[(2R)-1-(prop-2-enoyl)piperidin-2-yl]ethynyl}pyridin-4-yl)-1H,5H,6H,7H-pyrrolo[3,2-c]pyridin-4-one (10.2 mg, 20.32%) as a yellow solid.

LC-MS: (M+H)⁺ found: 514.15.

¹H NMR (400 MHz, Chloroform-d) δ 10.48 (s, 1H), 8.60 (s, 1H), 8.14 (d, 1H), 7.84 (s, 1H), 7.42 (d, 1H), 6.67-6.55 (m, 2H), 6.55-6.46 (m, 1H), 6.36-6.27 (m, 1H), 6.09-6.02 (m, 1H), 5.81-5.73 (m, 1H), 5.64 (s, 1H), 5.30 (s, 1H), 4.10 (d, 3H), 3.99-3.93 (m, 1H), 3.68-3.54 (m, 2H), 3.42-3.33 (m, 1H), 3.20 (s, 2H), 2.10-2.08 (m, 1H), 1.91-1.83 (m, 4H), 1.62 (s, 1H).

### Example 144. 3-[(3-fluoro-2-methoxyphenyl)amino]-2-(3-{2-[(2S)-2-methyl-1-(prop-2-enoyl)pyrrolidin-2-yl]ethynyl}pyridin-4-yl)-1H,5H,6H,7H-pyrrolo[3,2-c]pyridin-4-one (compound 246)

### 144.1. Synthesis of tert-butyl (2S)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate

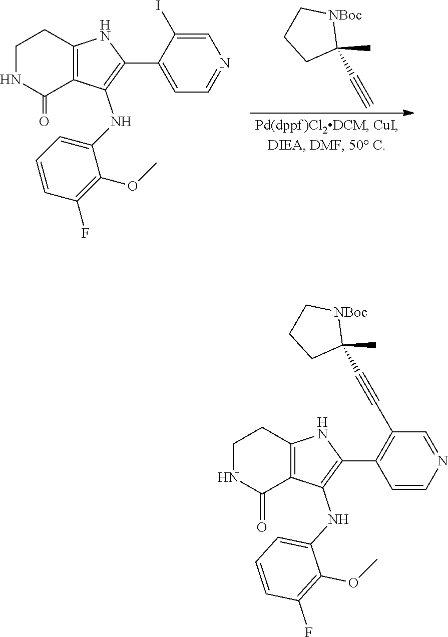 [SMILES: COc1c(F)cccc1Nc1c(-c2ccncc2I)[nH]c2c1C(=O)NCC2 ; C#C[C@]1(C)CCCN1C(=O)OC(C)(C)C ; COc1c(F)cccc1Nc1c(-c2ccncc2C#C[C@]2(C)CCCN2C(=O)OC(C)(C)C)[nH]c2c1C(=O)NCC2]

To a solution of 3-[(3-fluoro-2-methoxyphenyl) amino]-2-(3-iodopyridin-4-yl)-1H,5H,6H,7H-pyrrolo[3,2-c]pyridin-4-one (130 mg, 0.27 mmol, 1.00 equiv) and tert-butyl (2S)-2-ethynyl-2-methylpyrrolidine-1-carboxylate (114 mg, 0.54 mmol, 2.00 equiv) in DMF (2.00 mL) were added CuI (26 mg, 0.14 mmol, 0.50 equiv), Pd(dppf)Cl₂CH₂Cl₂ (55 mg, 0.07 mmol, 0.25 equiv) and DIEA (88 mg, 0.68 mmol, 2.50 equiv). The resulting suspension was backfilled with argon three times and stirred for 2 h at 50° C. LCMS confirmed completion of reaction and desired product was observed. The resulting mixture was purified by reverse flash chromatography with the following conditions (column, C18 gel; mobile phase, ACN in water, 10% to 100% gradient in 10 min; detector, UV 254 nm) to afford tert-butyl (2S)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (100 mg, 62.45%) as a yellow solid.

LC-MS: (M+H)⁺ found: 560.20.

### 144.2. Synthesis of 3-[(3-fluoro-2-methoxyphenyl)amino]-2-(3-{2-[(2S)-2-methylpyrrolidin-2-yl]ethynyl}pyridin-4-yl)-1H,5H,6H,7H-pyrrolo[3,2-c]pyridin-4-one

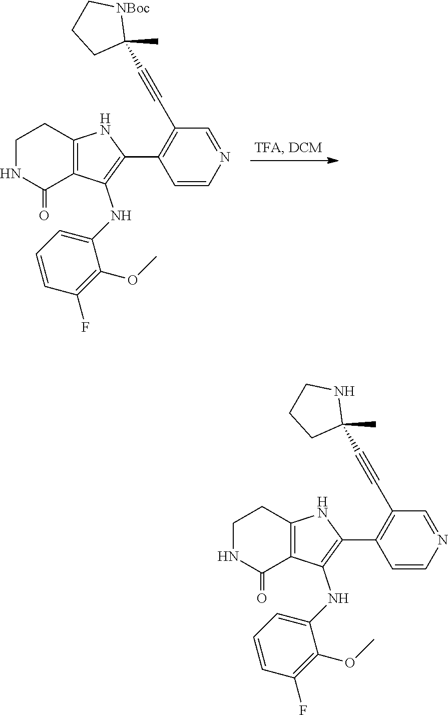 [SMILES: COc1c(F)cccc1Nc1c(-c2ccncc2C#C[C@]2(C)CCCN2C(=O)OC(C)(C)C)[nH]c2c1C(=O)NCC2 ; COc1c(F)cccc1Nc1c(-c2ccncc2C#C[C@]2(C)CCCN2)[nH]c2c1C(=O)NCC2]

To a stirred mixture of tert-butyl (2S)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (80 mg, 0.14 mmol) in DCM (1.00 mL) was added TFA (1.00 mL) at room temperature under nitrogen atmosphere. The resulting mixture was stirred for 1 h and concentrated under reduced pressure to 3-[(3-fluoro-2-methoxyphenyl)amino]-2-(3-{2-[(2S)-2-methylpyrrolidin-2-yl]ethynyl}pyridin-4-yl)-1H,5H,6H,7H-pyrrolo[3,2-c]pyridin-4-one (80 mg, crude) as a red oil

LC-MS: (M+H)⁺ found: 460.45.

### 144.3. Synthesis of 3-[(3-fluoro-2-methoxyphenyl)amino]-2-(3-{2-[(2S)-2-methyl-1-(prop-2-enoyl)pyrrolidin-2-yl]ethynyl}pyridin-4-yl)-1H,5H,6H,7H-pyrrolo[3,2-c]pyridin-4-one

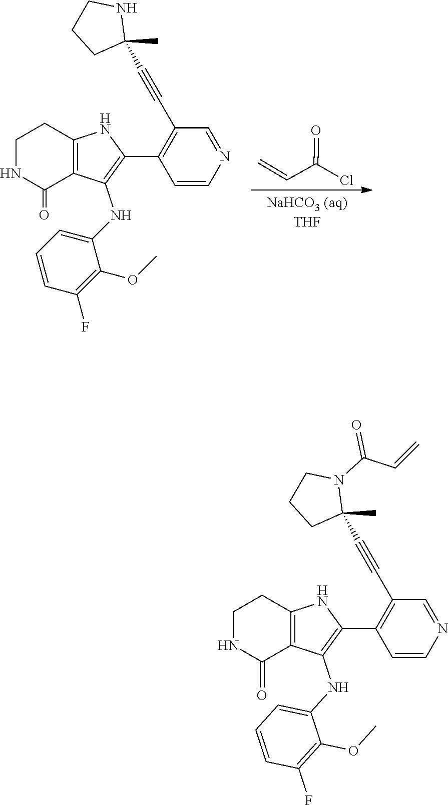

A solution of 3-[(3-fluoro-2-methoxyphenyl)amino]-2-(3-{2-[(2S)-2-methylpyrrolidin-2-yl]ethynyl}pyridin-4-yl)-1H,5H,6H,7H-pyrrolo[3,2-c]pyridin-4-one (80 mg, 0.17 mmol, 1.00 equiv) in THF (1.00 mL) was basified to pH 8 with NaHCO₃(aq) (1.00 mL). To the above mixture was added acryloyl chloride (14 mg, 0.18 mmol, 0.90 equiv) at 0° C. under nitrogen atmosphere dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product (60 mg) that was purified by Prep-HPLC under the following conditions (Column: Xselect CSH F-Phenyl OBD column, 30*250 mm, 5 μm; Mobile Phase A: Water (0.1% FA), Mobile Phase B: ACN; Flow rate: 60 mL/min; Gradient: 32% B to 62% B in 7 min; Wave Length: 254 nm; RT1(min): 6.5; Number Of Runs: 0) to afford 3-[(3-fluoro-2-methoxyphenyl)amino]-2-(3-{2-[(2S)-2-methyl-1-(prop-2-enoyl)pyrrolidin-2-yl]ethynyl}pyridin-4-yl)-1H,5H,6H,7H-pyrrolo[3,2-c]pyridin-4-one (9.9 mg, 10.63%) as a yellow solid.

LC-MS: (M+H)⁺ found: 460.45.

¹H NMR (400 MHz, Chloroform-d) δ 11.49 (s, 1H), 8.52 (s, 1H), 8.14 (d, 1H), 7.68 (s, 1H), 7.46 (d, 1H), 6.59-6.56 (m, 1H), 6.52-6.44 (m, 2H), 6.39-6.35 (m, 1H), 6.12-6.10 (m, 1H), 5.75-5.72 (m, 1H), 5.24 (s, 1H), 4.10 (s, 3H), 3.80-3.74 (m, 2H), 3.63-3.60 (m, 2H), 3.29-3.22 (m, 2H), 2.19-2.14 (m, 4H), 1.78 (s, 3H).

### Example 145. 3-[(3-fluoro-2-methoxyphenyl)amino]-2-(3-{2-[(2R)-2-methyl-1-(prop-2-enoyl)pyrrolidin-2-yl]ethynyl}pyridin-4-yl)-1H,5H,6H,7H-pyrrolo[3,2-c]pyridin-4-one (compound 245)

### 145.1. Synthesis of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate

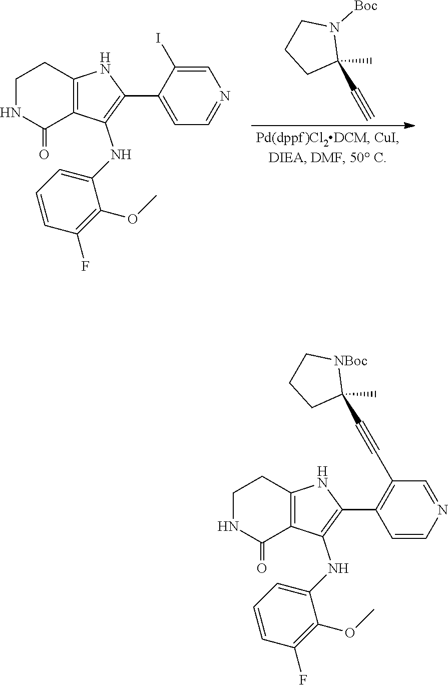 [SMILES: COc1c(F)cccc1Nc1c(-c2ccncc2I)[nH]c2c1C(=O)NCC2 ; C#C[C@@]1(C)CCCN1C(=O)OC(C)(C)C ; COc1c(F)cccc1Nc1c(-c2ccncc2C#C[C@@]2(C)CCCN2C(=O)OC(C)(C)C)[nH]c2c1C(=O)NCC2]

To a stirred mixture of 3-[(3-fluoro-2-methoxyphenyl)amino]-2-(3-iodopyridin-4-yl)-1H,5H,6H,7H-pyrrolo[3,2-c]pyridin-4-one (150 mg, 0.31 mmol, 1.00 equiv) and Pd(dppf)C12-CH₂Cl₂ (63 mg, 0.08 mmol, 0.25 equiv) in DMF (2.00 mL) was added 5 DIEA (202 mg, 1.58 mmol, 5.00 equiv) and tert-butyl (2R)-2-ethynyl-2-methylpyrrolidine-1-carboxylate (196 mg, 0.94 mmol, 3.00 equiv) at room temperature under argon atmosphere. The resulting suspension was backfilled with argon three times and stirred for 2 h at 50° C. LCMS confirmed completion of reaction and desired product was observed. The residue was purified by reverse flash chromatography with the following conditions: column, C18 gel; mobile phase, MeCN in water, 10% to 70% gradient in 20 min; detector, UV 254 nm. This resulted in tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (150 mg, 85.46%) as a yellow solid.

LC-MS: (M+H)⁺ found: 560.1

### 145.2. Synthesis of 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one

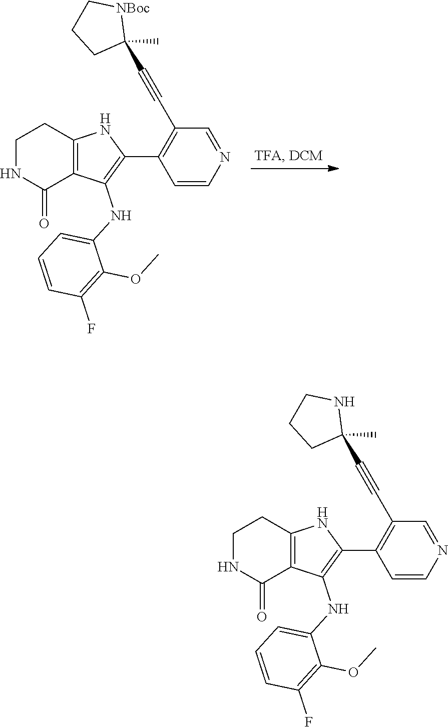 [SMILES: COc1c(F)cccc1Nc1c(-c2ccncc2C#C[C@@]2(C)CCCN2C(=O)OC(C)(C)C)[nH]c2c1C(=O)NCC2 ; COc1c(F)cccc1Nc1c(-c2ccncc2C#C[C@@]2(C)CCCN2)[nH]c2c1C(=O)NCC2]

To a stirred mixture of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (120 mg, 0.21 mmol) in DCM (3.00 mL) was added TFA (1.00 mL) at room temperature under nitrogen atmosphere. The resulting mixture was stirred for 1 h and concentrated under reduced pressure to afford 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (98 mg, crude) as a red oil.

LC-MS: (M+H)⁺ found: 460.1

### 145.3. Synthesis of 3-[(3-fluoro-2-methoxyphenyl)amino]-2-(3-{2-[(2R)-2-methyl-1-(prop-2-enoyl)pyrrolidin-2-yl]ethynyl}pyridin-4-yl)-1H,5H,6H,7H-pyrrolo[3,2-c]pyridin-4-one

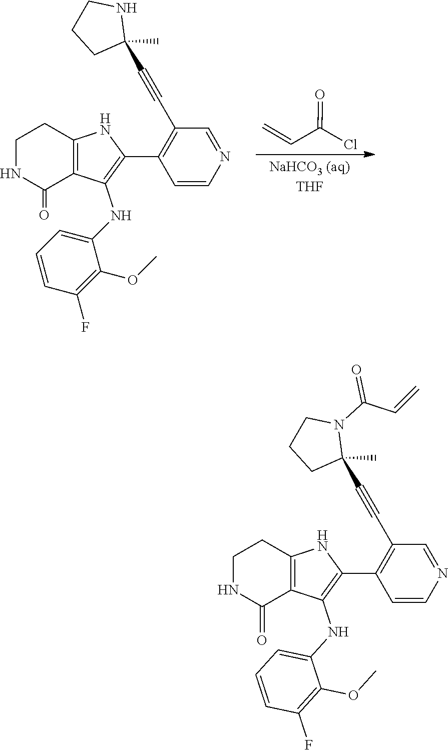

A solution of 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (98 mg, 0.21 mmol, 1.00 equiv) in THF (3.00 mL) was basified to pH 8 with NaHCO₃aq (3.00 mL). To the above mixture was added acryloyl chloride (19 mg, 0.21 mmol, 1.00 equiv) at 0° C. under nitrogen atmosphere dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product (120 mg) that was purified by Prep-HPLC under the following conditions (Column: Xcelect CSH F-pheny OBD Column, 19*250 mm, 5 μm; Mobile Phase A: Water (10 mmol/L NH₄HCO₃), Mobile Phase B: ACN; Flow rate: 25 mL/min; Gradient: 40% B to 60% B in 10 min, 60% B; Wave Length: 254/220 nm; RT1(min): 8.32; Number Of Runs: 0) to afford 3-[(3-fluoro-2-methoxyphenyl)amino]-2-(3-{2-[(2R)-2-methyl-1-(prop-2-enoyl)pyrrolidin-2-yl]ethynyl}pyridin-4-yl)-1H,5H,6H,7H-pyrrolo[3,2-c]pyridin-4-one (16.5 mg, 13.91%) as a yellow solid

LC-MS: (M+H)⁺ found: 514.10

¹H NMR (300 MHz, Chloroform-d) δ 11.49 (s, 1H), 8.52 (s, 1H), 8.14 (s, 1H), 7.71 (s, 1H), 7.47 (s, 1H), 6.64-6.52 (m, 1H), 6.51-6.32 (m, 3H), 6.10 (d, J=8.1 Hz, 1H), 5.78-5.70 (m, 1H), 5.19 (s, 1H), 4.10 (s 3H), 3.85-3.68 (m, 2H), 3.64-3.60 (m, 2H), 3.37-3.13 (m, 2H), 2.58-2.43 (m, 1H), 2.23-2.03 (m, 3H), 1.78 (s, 3H).

### Example 146. rel-3-[(3-fluoro-2-methoxyphenyl)amino]-2-(3-{2-[(3R)-4-(prop-2-enoyl)morpholin-3-yl]ethynyl}pyridin-4-yl)-1H,5H,6H,7H-pyrrolo[3,2-c]pyridin-4-one (compound 242)

### 146.1. Synthesis of tert-butyl 3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]morpholine-4-carboxylate

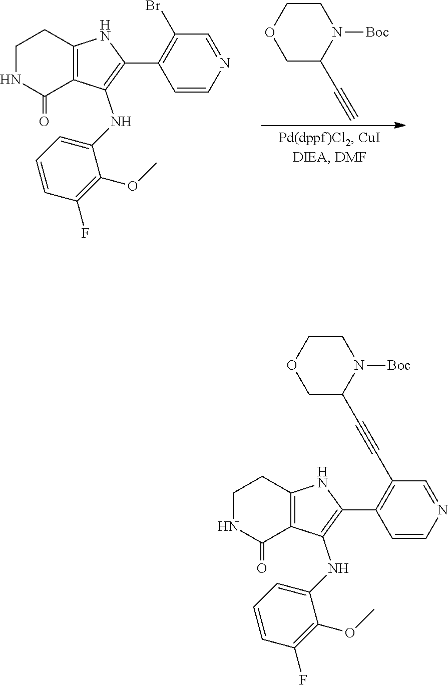 [SMILES: COc1c(F)cccc1Nc1c(-c2ccncc2Br)[nH]c2c1C(=O)NCC2 ; C#CC1COCCN1C(=O)OC(C)(C)C ; COc1c(F)cccc1Nc1c(-c2ccncc2C#CC2COCCN2C(=O)OC(C)(C)C)[nH]c2c1C(=O)NCC2]

To a stirred solution/mixture of 2-(3-bromopyridin-4-yl)-3-[(3-fluoro-2-methoxyphenyl)amino]-1H,5H,6H,7H-pyrrolo[3,2-c]pyridin-4-one (320 mg, 0.74 mmol, 1.00 equiv) and tert-butyl 3-ethynylmorpholine-4-carboxylate (470 mg, 2.22 mmol, 3.00 equiv) in DMF (7.00 mL) were added CuI (71 mg, 0.371 mmol, 0.50 equiv), Pd(dppf)C12-CH₂Cl₂ (302 mg, 0.37 mmol, 0.50 equiv) and DIEA (288 mg, 2.22 mmol, 3.00 equiv) at room temperature. The resulting suspension was backfilled with argon three times and stirred for 1.5 h at 50° C. LCMS confirmed completion of reaction and desired product was observed. The resulting mixture was purified by reverse flash chromatography with the following conditions (column, silica gel; mobile phase, MeCN in water, 10% to 50% gradient in 10 min; detector, UV 254 nm) to afford tert-butyl 3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]morpholine-4-carboxylate (230 mg, 55.19%) as a yellow solid.

LC-MS: M+H found: 562.3.

### 146.2. Synthesis of 3-[(3-fluoro-2-methoxyphenyl)amino]-2-{3-[2-(morpholin-3-yl)ethynyl]pyridin-4-yl}-1H,5H,6H,7H-pyrrolo[3,2-c]pyridin-4-one

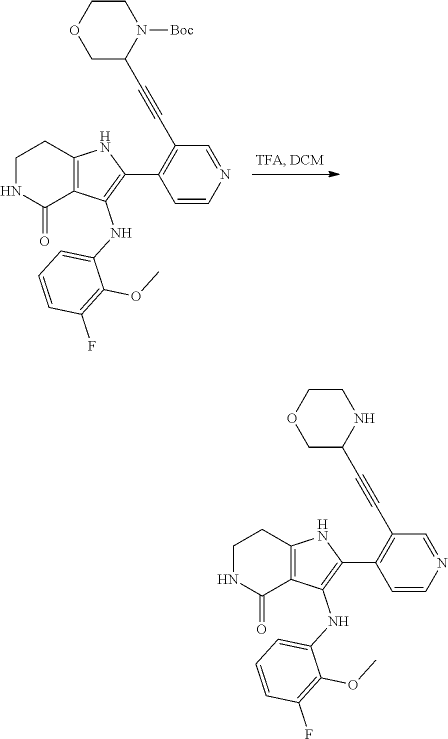 [SMILES: COc1c(F)cccc1Nc1c(-c2ccncc2C#CC2COCCN2C(=O)OC(C)(C)C)[nH]c2c1C(=O)NCC2 ; COc1c(F)cccc1Nc1c(-c2ccncc2C#CC2COCCN2)[nH]c2c1C(=O)NCC2]

To a stirred mixture of tert-butyl 3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]morpholine-4-carboxylate (200 mg, 0.356 mmol) in DCM (4.00 mL) was added TFA (2.00 mL) at room temperature under nitrogen atmosphere. The resulting mixture was stirred for 1 h and concentrated under reduced pressure to afford 3-[(3-fluoro-2-methoxyphenyl)amino]-2-{3-[2-(morpholin-3-yl)ethynyl]pyridin-4-yl}-1H,5H,6H,7H-pyrrolo[3,2-c]pyridin-4-one (164 mg, crude) as a red oil.

LC-MS: (M+H)⁺ found 462.1.

### 146.3. Synthesis of 3-[(3-fluoro-2-methoxyphenyl)amino]-2-(3-{2-[4-(prop-2-enoyl)morpholin-3-yl]ethynyl}pyridin-4-yl)-1H,5H,6H,7H-pyrrolo[3,2-c]pyridin-4-one

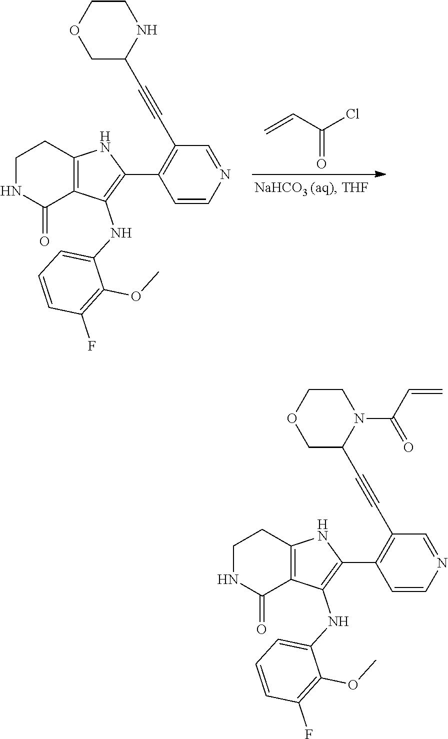

A solution of 3-[(3-fluoro-2-methoxyphenyl)amino]-2-{3-[2-(morpholin-3-yl)ethynyl]pyridin-4-yl}-1H,5H,6H,7H-pyrrolo[3,2-c]pyridin-4-one (164 mg, 0.35 mmol, 1.00 equiv) in THF (3.00 mL) was basified to pH 8 with NaHCO₃aq (2.00 mL). To the above mixture was added acryloyl chloride (51 mg, 0.57 mmol, 1.60 equiv) at 0° C. under nitrogen atmosphere dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product (60 mg) that was purified by Prep-HPLC under the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 24% B to 54% B in 7 min; Wave Length: 254 nm; RT1(min): 6.5; Number Of Runs: 0) to afford 3-[(3-fluoro-2-methoxyphenyl)amino]-2-(3-{2-[4-(prop-2-enoyl)morpholin-3-yl]ethynyl}pyridin-4-yl)-1H,5H,6H,7H-pyrrolo[3,2-c]pyridin-4-one (50 mg, 27.66%) as a yellow solid.

LC-MS: (M+H)⁺ found 530.15.

### 146.4. Synthesis of rel-3-[(3-fluoro-2-methoxyphenyl)amino]-2-(3-{2-[(3R)-4-(prop-2-enoyl)morpholin-3-yl]ethynyl}pyridin-4-yl)-1H,5H,6H,7H-pyrrolo[3,2-c]pyridin-4-one

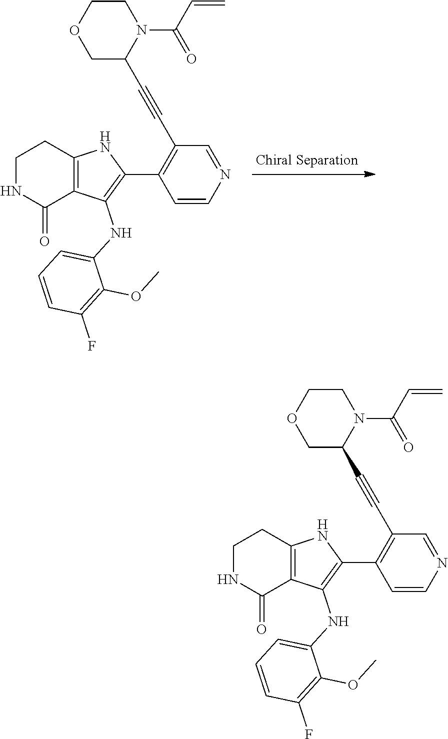 [SMILES: C=CC(=O)N1CCOCC1C#Cc1cnccc1-c1[nH]c2c(c1Nc1cccc(F)c1OC)C(=O)NCC2 ; C=CC(=O)N1CCOC[C@@H]1C#Cc1cnccc1-c1[nH]c2c(c1Nc1cccc(F)c1OC)C(=O)NCC2]

The crude product (50 mg) was separated by Chiral-HPLC with the following conditions (Column: CHIRALPAK IG-3, 4.6*50 mm, 3 m; Mobile Phase A: (Hex:DCM=3:1)(0.1% DEA):EtOH=80:20; Flow rate: 1 mL/min; Gradient: 0% B to 0% B; Injection Volume: 5 ul mL) to afford rel-3-[(3-fluoro-2-methoxyphenyl)amino]-2-(3-{2-[(3R)-4-(prop-2-enoyl)morpholin-3-yl]ethynyl}pyridin-4-yl)-1H,5H,6H,7H-pyrrolo[3,2-c]pyridin-4-one (11.4 mg, 21.88%) as a light yellow solid.

LC-MS: (M+H)⁺ found 516.10.

¹H NMR (400 MHz, Chloroform-d) δ 10.54 (s, 1H), 8.64 (s, 1H), 8.17 (d, 1H), 7.96 (s, 1H), 7.45 (d, 1H), 6.63-6.40 (m, 4H), 6.08 (d, 1H), 5.88 (d, 1H), 5.53 (s, 1H), 5.28 (s, 1H), 4.24 (m, 1H), 4.13 (m, 4H), 3.75-3.60 (m, 6H), 3.15 (s, 2H).

### Example 147. rel-3-[(3-fluoro-2-methoxyphenyl)amino]-2-(3-{2-[(3R)-4-(prop-2-enoyl)morpholin-3-yl]ethynyl}pyridin-4-yl)-1H,5H,6H,7H-pyrrolo[3,2-c]pyridin-4-one (compound 241)

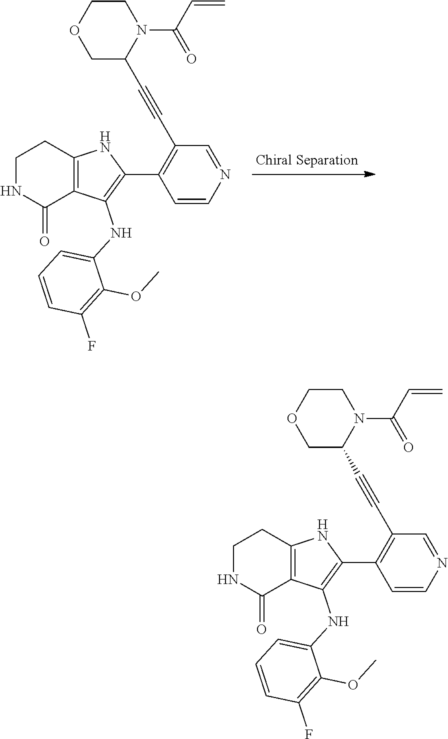 [SMILES: C=CC(=O)N1CCOCC1C#Cc1cnccc1-c1[nH]c2c(c1Nc1cccc(F)c1OC)C(=O)NCC2 ; C=CC(=O)N1CCOC[C@H]1C#Cc1cnccc1-c1[nH]c2c(c1Nc1cccc(F)c1OC)C(=O)NCC2]

The crude product (50 mg) was separated by Chiral-HPLC with the following conditions (Column: CHIRALPAK IG-3, 4.6*50 mm, 3 m; Mobile Phase A: (Hex:DCM=3:1)(0.1% DEA):EtOH=80:20; Flow rate: 1 mL/min; Gradient: 0% B to 0% B; Injection Volume: 5 ul mL) to afford rel-3-[(3-fluoro-2-methoxyphenyl)amino]-2-(3-{2-[(3R)-4-(prop-2-enoyl)morpholin-3-yl]ethynyl}pyridin-4-yl)-1H,5H,6H,7H-pyrrolo[3,2-c]pyridin-4-one (13.1 mg, 25.48%) as a light yellow solid.

LC-MS: (M+H)⁺ found 516.10.

¹H NMR (400 MHz, Chloroform-d) δ 10.78 (s, 1H), 8.59 (s, 1H), 8.51 (s, 1H), 7.98 (d, 1H), 7.40 (d, 1H), 6.67-6.55 (m, 3H), 6.45 (m, 1H), 6.07-6.00 (m, 1H), 5.91 (d, 1H), 5.38 (s, 2H), 4.24 (d, 1H), 4.15 (d, 3H), 4.10-4.07 (m, 1H), 3.87-3.83 (m, 1H), 3.77-3.66 (m 5H), 3.24 (s, 2H).

### Example 148. N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylprop-2-enamide (compound 260)

### 148.1. Synthesis of N-methyl-N-(2-methylbut-3-yn-2-yl)carbamate

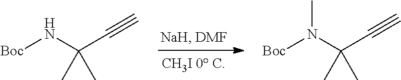

To a stirred mixture of tert-butyl N-(2-methylbut-3-yn-2-yl)carbamate (310 mg, 1.69 mmol, 1.00 equiv) in DMF (5.00 mL) were added NaH (81 mg, 3.38 mmol, 2.00 equiv) at 0° C. The resulting mixture was stirred for 0.5 h at 0° C. To the above mixture was added methyl iodide (480 mg, 3.38 mmol, 2.00 equiv) dropwise at 0° C. The resulting mixture was stirred for additional 2 h at 0° C. The reaction was quenched by the addition of H₂O (2 mL) at 0° C. The resulting mixture was extracted with EA (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EA (5/1) to afford tert-butyl N-methyl-N-(2-methylbut-3-yn-2-yl)carbamate (187 mg, 53.23%) as a colorless oil.

LC-MS: (M+H−56)⁺ found: 142.1. 148.2. Synthesis of tert-butyl N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylcarbamate

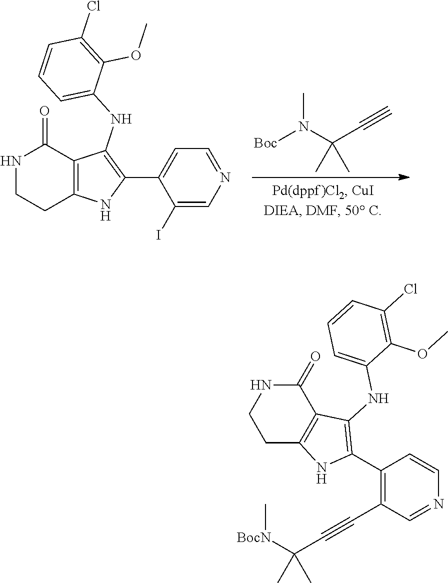 [SMILES: COc1c(Cl)cccc1Nc1c(-c2ccncc2I)[nH]c2c1C(=O)NCC2 ; C#CC(C)(C)N(C)C(=O)OC(C)(C)C ; COc1c(Cl)cccc1Nc1c(-c2ccncc2C#CC(C)(C)N(C)C(=O)OC(C)(C)C)[nH]c2c1C(=O)NCC2]

To a solution of 3-[(3-chloro-2-methoxyphenyl)amino]-2-(3-iodopyridin-4-yl)-1H,5H,6H,7H-pyrrolo[3,2-c]pyridin-4-one (100 mg, 0.20 mmol, 1.00 equiv) and tert-butyl N-methyl-N-(2-methylbut-3-yn-2-yl)carbamate (80 mg, 0.40 mmol, 2.00 equiv) in DMF (2.00 mL) were added DIEA (65 mg, 0.50 mmol, 2.50 equiv), CuI (19 mg, 0.10 mmol, 0.50 equiv) and Pd(dppf)C12 CH₂Cl₂ (41 mg, 0.05 mmol, 0.25 equiv). The resulting suspension was backfilled with argon three times and stirred for 2 h at 50° C. LCMS confirmed completion of reaction and desired product was observed. The resulting mixture was concentrated under vacuum. The residue was purified by reverse flash chromatography with the following conditions (column, C18 gel; mobile phase, ACN in water, 10% to 100% gradient in 10 min; detector, UV 254 nm) to afford tert-butyl N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylcarbamate (70 mg, 58.32%) as a yellow solid.

LC-MS: (M+H)⁺ found: 564.10.

### 148.3. Synthesis of 3-[(3-chloro-2-methoxyphenyl)amino]-2-{3-[3-methyl-3-(methylamino)but-1-yn-1-yl]pyridin-4-yl}-1H,5H,6H,7H-pyrrolo[3,2-c]pyridin-4-one

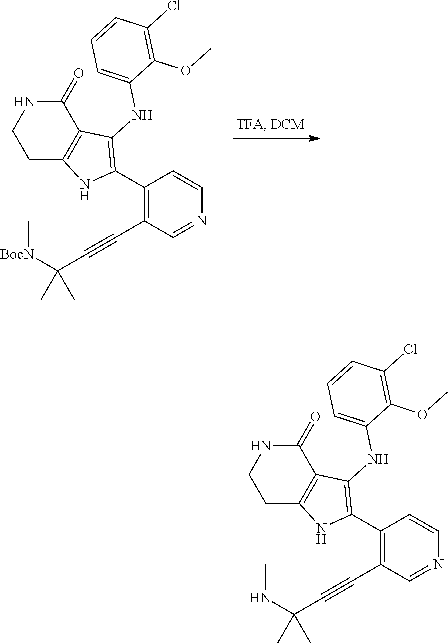 [SMILES: COc1c(Cl)cccc1Nc1c(-c2ccncc2C#CC(C)(C)N(C)C(=O)OC(C)(C)C)[nH]c2c1C(=O)NCC2 ; CNC(C)(C)C#Cc1cnccc1-c1[nH]c2c(c1Nc1cccc(Cl)c1OC)C(=O)NCC2]

Into a 8 mL Vessel were added tert-butyl N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylcarbamate (80 mg, 0.14 mmol) and TFA (2.00 mL) at room temperature. The resulting mixture was stirred for 15 h and dried using nitrogen to afford 3-[(3-chloro-2-methoxyphenyl)amino]-2-{3-[3-methyl-3-(methylamino)but-1-yn-1-yl]pyridin-4-yl}-1H,5H,6H,7H-pyrrolo[3,2-c]pyridin-4-one (80 mg, crude) as a red oil

LC-MS: (M+H)⁺ found: 464.45

### 148.4. Synthesis of N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylprop-2-enamide

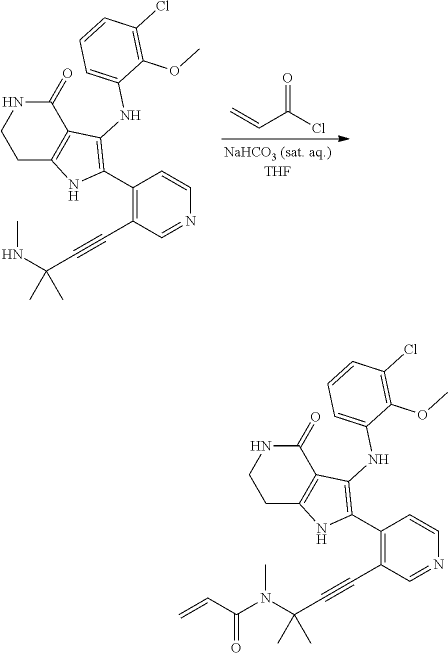

To a stirred solution of 3-[(3-chloro-2-methoxyphenyl)amino]-2-{3-[3-methyl-3-(methylamino)but-1-yn-1-yl]pyridin-4-yl}-1H,5H,6H,7H-pyrrolo[3,2-c]pyridin-4-one (80 mg, 0.17 mmol, 1.00 equiv) in THF (1.00 mL) were added NaHCO₃aq. (1.00 mL) and acryloyl chloride (14 mg, 0.15 mmol, 0.90 equiv) dropwise at 0° C. The resulting mixture was stirred for 1 h at room temperature. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 37% B to 58% B in 9 min, 58% B; Wave Length: 254/220 nm; RT1(min): 8.85; Number Of Runs: 0) to afford N-[4-(4-{3-[(3-chloro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylprop-2-enamide (4.3 mg, 4.66%) as a yellow solid.

LC-MS: (M+H)⁺ found: 517.19.

¹H NMR (400 MHz, DMSO-d6) δ 11.75 (s, 1H), 8.53 (s, 1H), 8.25 (d, 1H), 7.63 (s, 1H), 7.36 (d, 1H), 7.21 (s, 1H), 6.84-6.77 (m, 1H), 6.72 (d, 2H), 6.20-6.13 (m, 2H), 5.74-5.71 (m, 1H), 3.89 (s, 3H), 3.51-3.42 (m, 2H), 3.09 (s, 3H), 3.01-2.91 (m, 2H), 1.72 (s, 6H).

### Example 149. N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylprop-2-enamide (compound 259)

### 149.1. Synthesis of tert-butyl N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylcarbamate

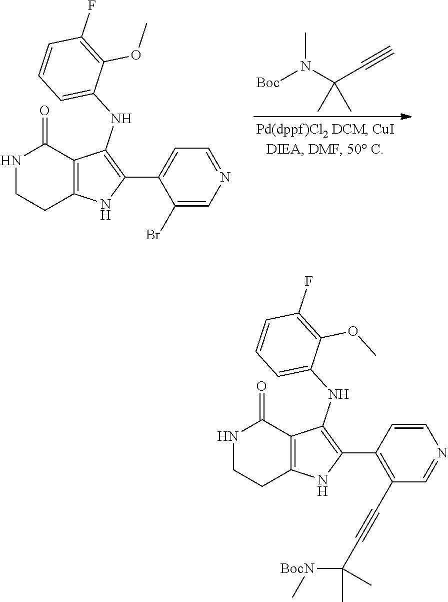 [SMILES: COc1c(F)cccc1Nc1c(-c2ccncc2Br)[nH]c2c1C(=O)NCC2 ; C#CC(C)(C)N(C)C(=O)OC(C)(C)C ; COc1c(F)cccc1Nc1c(-c2ccncc2C#CC(C)(C)N(C)C(=O)OC(C)(C)C)[nH]c2c1C(=O)NCC2]

To a stirred mixture of 2-(3-bromopyridin-4-yl)-3-[(3-fluoro-2-methoxyphenyl)amino]-1H,5H,6H,7H-pyrrolo[3,2-c]pyridin-4-one (250 mg, 0.58 mmol, 1.00 equiv) and tert-butyl N-methyl-N-(2-methylbut-3-yn-2-yl)carbamate (229 mg, 1.16 mmol, 2.00 equiv) in DMF (2.00 mL) were added DIEA (187 mg, 1.45 mmol, 2.50 equiv), CuI (55 mg, 0.29 mmol, 0.50 equiv) and Pd(dppf)Cl₂·CH₂Cl₂ (118 mg, 0.15 mmol, 0.25 equiv) under argon atmosphere. The resulting suspension was backfilled with argon three times and stirred for 2 h at 50° C. LCMS confirmed completion of reaction and desired product was observed. The mixture was purified by reverse flash chromatography with the following conditions (column, C18 gel; mobile phase, MeCN in water, 10% to 50% gradient in 10 min; detector, UV 254 nm) to afford tert-butyl N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylcarbamate (170 mg, 48.20%) as a yellow solid.

LC-MS: (M+H)⁺ found: 548.25.

### 149.2. Synthesis of 3-[(3-fluoro-2-methoxyphenyl)amino]-2-{3-[3-methyl-3-(methylamino)but-1-yn-1-yl]pyridin-4-yl}-1H,5H,6H,7H-pyrrolo[3,2-c]pyridin-4-one

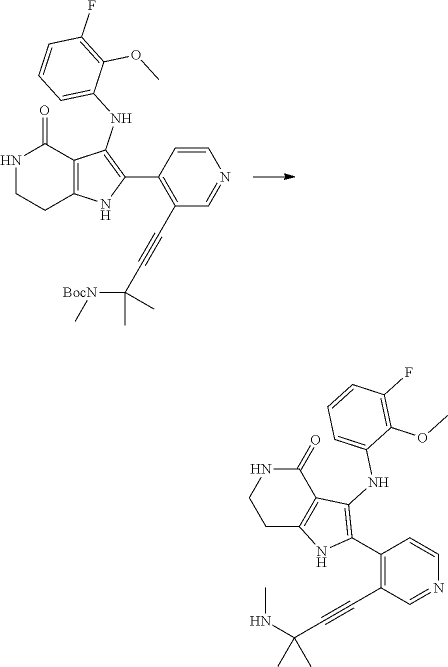 [SMILES: COc1c(F)cccc1Nc1c(-c2ccncc2C#CC(C)(C)N(C)C(=O)OC(C)(C)C)[nH]c2c1C(=O)NCC2 ; CNC(C)(C)C#Cc1cnccc1-c1[nH]c2c(c1Nc1cccc(F)c1OC)C(=O)NCC2]

To a stirred solution of 2,6-lutidine (685 mg, 6.40 mmol, 25.00 equiv) and tert-butyl N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylcarbamate (140 mg, 0.26 mmol, 1.00 equiv) in DCM (5.00 mL) was added TMSOTf (0.50 mL, 1.30 mmol, 5.00 equiv) dropwise at 0° C. The resulting mixture was stirred for 10 min at 0° C., then stirred for 60 min at 25° C. The reaction was monitored by LCMS. The resulting mixture was concentrated under vacuum. The residue was purified by reverse flash chromatography with the following conditions (column, C18 silica gel; mobile phase, ACN in water, 10% to 50% gradient in 10 min; detector, UV 254 nm) to afford 3-[(3-fluoro-2-methoxyphenyl)amino]-2-{3-[3-methyl-3-(methylamino)but-1-yn-1-yl]pyridin-4-yl}-1H,5H,6H,7H-pyrrolo[3,2-c]pyridin-4-one (80 mg, 62.93%) as a light yellow solid.

LC-MS: (M+H)⁺ found: 448.10.

### 149.3. Synthesis of N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylprop-2-enamide

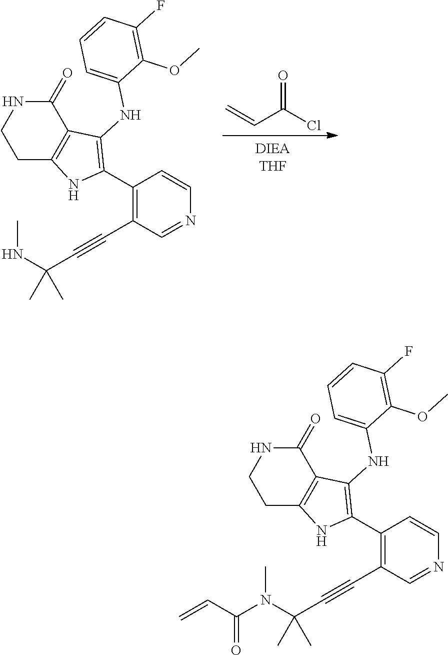

To a stirred solution of 3-[(3-fluoro-2-methoxyphenyl)amino]-2-{3-[3-methyl-3-(methylamino)but-1-yn-1-yl]pyridin-4-yl}-1H,5H,6H,7H-pyrrolo[3,2-c]pyridin-4-one (70 mg, 0.16 mmol, 1.00 equiv) and DIEA (61 mg, 0.47 mmol, 3.00 equiv) in tetrahydrofuran (1.00 mL) was added acryloyl chloride (13 mg, 0.14 mmol, 0.90 equiv) dropwise at 0° C. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 32% B to 57% B in 9 min, 57% B; Wave Length: 254/220 nm; RT1(min): 8.85; Number Of Runs: 0) to afford N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]-N-methylprop-2-enamide (14.9 mg, 17.43%) as a off-white solid.

LC-MS: (M+H)⁺ found: 502.10.

¹H NMR (300 MHz, DMSO-d6) δ 11.74 (s, 1H), 8.52 (s, 1H), 8.24 (d, 1H), 7.64 (s, 1H), 7.36 (d, 1H), 7.22 (s, 1H), 6.85-6.76 (m, 1H), 6.70-6.65 (m, 1H), 6.63-6.42 (m, 1H), 6.20-6.15 (m, 1H), 6.14-5.98 (m, 1H), 5.74-5.70 (m, 1H), 3.92 (s, 3H), 3.45-3.41 (m, 2H), 3.09 (s, 3H), 3.09-2.87 (m, 2H), 1.72 (s, 6H).

### Example 150. N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]prop-2-enamide (compound 258)

### 150.1. Synthesis of tert-butyl N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]carbamate

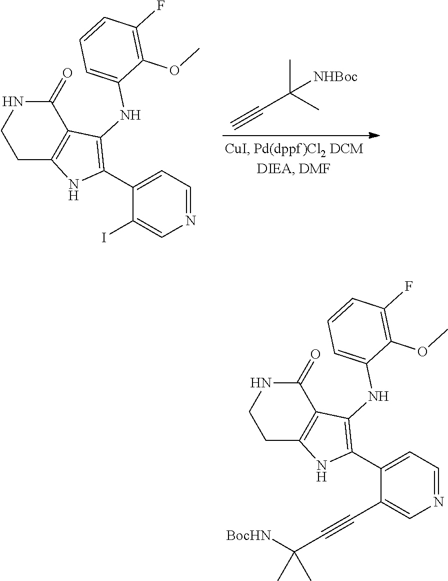 [SMILES: COc1c(F)cccc1Nc1c(-c2ccncc2I)[nH]c2c1C(=O)NCC2 ; C#CC(C)(C)NC(=O)OC(C)(C)C ; COc1c(F)cccc1Nc1c(-c2ccncc2C#CC(C)(C)NC(=O)OC(C)(C)C)[nH]c2c1C(=O)NCC2]

To a stirred mixture of 3-[(3-fluoro-2-methoxyphenyl)amino]-2-(3-iodopyridin-4-yl)-1H,5H,6H,7H-pyrrolo[3,2-c]pyridin-4-one (130 mg, 0.27 mmol, 1.00 equiv) and tert-butyl N-(2-methylbut-3-yn-2-yl)carbamate (99 mg, 0.54 mmol, 2.00 equiv) in DMF (3.00 mL) was added Pd(dppf)Cl₂CH₂Cl₂ (110 mg, 0.13 mmol, 0.50 equiv) and CuI (25 mg, 0.13 mmol, 0.50 equiv) and DIEA (210 mg, 1.63 mmol, 6.00 equiv) at room temperature under argon atmosphere. The resulting suspension was backfilled with argon three times and stirred for 2 h at 50° C. LCMS confirmed completion of reaction and desired product was observed. The resulting mixture was concentrated under reduced pressure. The residue was purified by reverse flash chromatography with the following conditions (column, C18 gel; mobile phase, MeCN in water, 0% to 100% gradient in 50 min; detector, UV 254 nm) To afford tert-butyl N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]carbamate (120 mg, 82.73%) as an off-white solid.

LC-MS: (M+H)⁺ found 534.1.

### 150.2. Synthesis of 2-[3-(3-amino-3-methylbut-1-yn-1-yl)pyridin-4-yl]-3-[(3-fluoro-2-methoxyphenyl)amino]-1H,5H,6H,7H-pyrrolo[3,2-c]pyridin-4-one

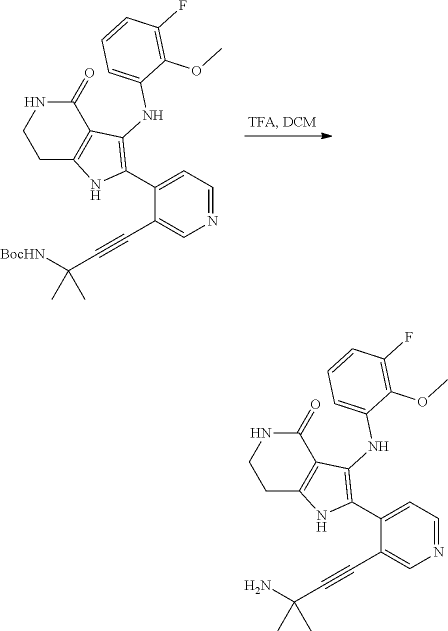 [SMILES: COc1c(F)cccc1Nc1c(-c2ccncc2C#CC(C)(C)NC(=O)OC(C)(C)C)[nH]c2c1C(=O)NCC2 ; COc1c(F)cccc1Nc1c(-c2ccncc2C#CC(C)(C)N)[nH]c2c1C(=O)NCC2]

To a stirred mixture of tert-butyl N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]carbamate (90 mg, 0.16 mmol, 1.00 equiv) in DCM (1.00 mL) was added TFA (1.00 mL) at 0° C. under nitrogen atmosphere. The resulting mixture was stirred for 1 h and dried using 5 nitrogen to afford 2-[3-(3-amino-3-methylbut-1-yn-1-yl)pyridin-4-yl]-3-[(3-fluoro-2-methoxyphenyl)amino]-1H,5H,6H,7H-pyrrolo[3,2-c]pyridin-4-one (90 mg, crude) as a red oil.

LC-MS: (M+H)⁺ found 434.1.

### 150.3. Synthesis of N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]prop-2-enamide

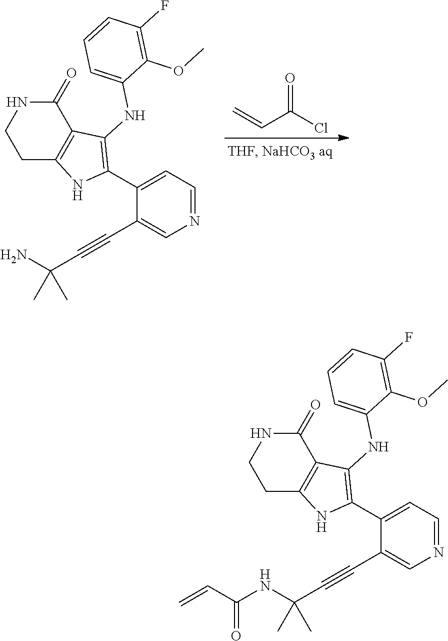

A solution of 2-[3-(3-amino-3-methylbut-1-yn-1-yl)pyridin-4-yl]-3-[(3-fluoro-2-methoxyphenyl)amino]-1H,5H,6H,7H-pyrrolo[3,2-c]pyridin-4-one (90 mg, 0.20 mmol, 1.00 equiv) in THF (1.00 mL) was basified to pH 8 with NaHCO₃(sat.) (1.00 mL). To the above mixture was acryloyl chloride (21 mg, 0.23 mmol, 1.15 equiv) at 0° C. under nitrogen atmosphere followed by the addition of T₃P (169 mg, 0.27 mmol, 2.00 equiv, 50% in EA) dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 26% B to 50% B in 9 min, 50% B; Wave Length: 254/220 nm; RT1(min): 8.85; Number Of Runs: 0) to afford N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]prop-2-enamide (5.0 mg, 4.88%) as an off-white solid.

LC-MS: (M+H)⁺ found 488.00.

¹H NMR (400 MHz, DMSO-d6) δ 11.63 (s, 1H), 8.75 (s, 1H), 8.51 (s, 1H), 8.23-8.21 (m, 1H), 7.65 (s, 1H), 7.36 (d, 1H), 7.21 (s, 1H), 6.67-6.66 (m, 1H), 6.55-6.52 (m, 1H), 6.32-6.19 (m, 2H), 5.99 (d, 1H), 5.70-5.67 (m, 1H), 3.92 (s, 3H), 3.44 (t, 2H), 3.02 (t, 2H), 1.64 (s, 6H).

### Example 151. N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxetan-3-yl}prop-2-enamide (compound 263) 151.1. Synthesis of tert-butyl N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxetan-3-yl}carbamate

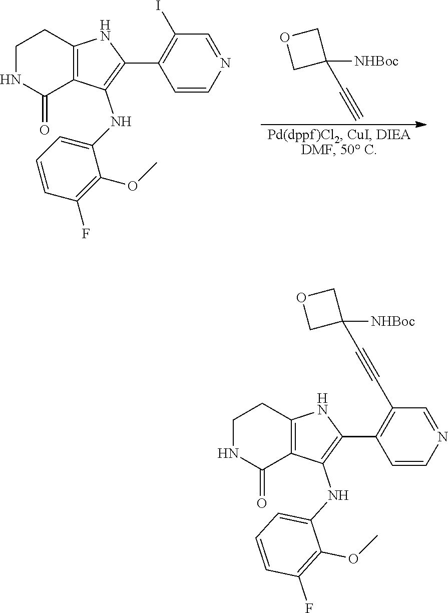

To a stirred mixture of 3-[(3-fluoro-2-methoxyphenyl)amino]-2-(3-iodopyridin-4-yl)-1H,5H,6H,7H-pyrrolo[3,2-c]pyridin-4-one (100 mg, 0.21 mmol, 1.00 equiv) and tert-butyl N-(3-ethynyloxetan-3-yl)carbamate (206 mg, 1.05 mmol, 5.00 equiv) in dimethylformamide (2.00 mL) were added copper(I) iodide (19 mg, 0.10 mmol, 0.50 equiv), DIEA (13 mg, 0.10 mmol, 0.50 equiv) and Pd(dppf)Cl₂·CH₂Cl₂ (42 mg, 0.05 mmol, 0.25 equiv) at room temperature under argon atmosphere. The resulting suspension was backfilled with argon three times and stirred for 2 h at 50° C. LCMS confirmed completion of reaction and desired product was observed. The residue was purified by reverse flash chromatography with the following conditions: column, C18 gel; mobile phase, MeCN in water, 10% to 70% gradient in 20 min; detector, UV 254 nm. This resulted in tert-butyl N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxetan-3-yl}carbamate (110 mg, 95.9%) as a yellow solid.

LC-MS: [M+H]⁺ found 548.25.

### 151.2. Synthesis of 2-{3-[2-(3-aminooxetan-3-yl)ethynyl]pyridin-4-yl}-3-[(3-fluoro-2-methoxyphenyl)amino]-1H,5H,6H,7H-pyrrolo[3,2-c]pyridin-4-one

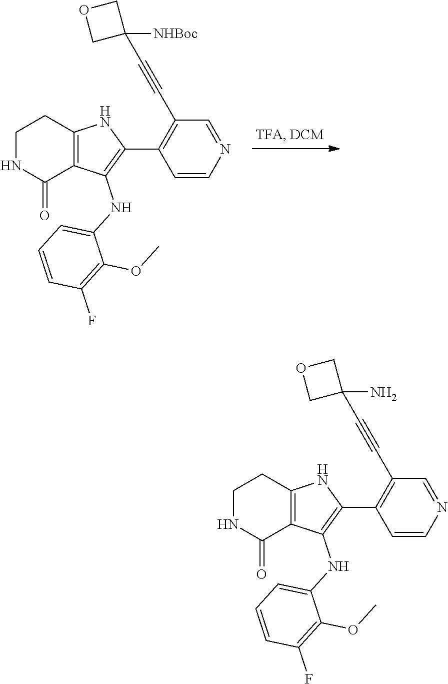 [SMILES: COc1c(F)cccc1Nc1c(-c2ccncc2C#CC2(NC(=O)OC(C)(C)C)COC2)[nH]c2c1C(=O)NCC2 ; COc1c(F)cccc1Nc1c(-c2ccncc2C#CC2(N)COC2)[nH]c2c1C(=O)NCC2]

To a stirred solution of tert-butyl N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxetan-3-yl}carbamate (90 mg, 0.16 mmol, 1.00 equiv) in DCM (2.00 mL) was added TFA (1.00 mL) dropwise at room temperature under argon atmosphere. The resulting mixture was stirred for 2 h at room temperature and dried using nitrogen to afford 2-{3-[2-(3-aminooxetan-3-yl)ethynyl]pyridin-4-yl}-3-[(3-fluoro-2-methoxyphenyl)amino]-1H,5H,6H,7H-pyrrolo[3,2-c]pyridin-4-one (73 mg, crude) as a red oil.

LC-MS: [M+H]⁺ found 448.15.

### 151.3. Synthesis of N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxetan-3-yl}prop-2-enamide

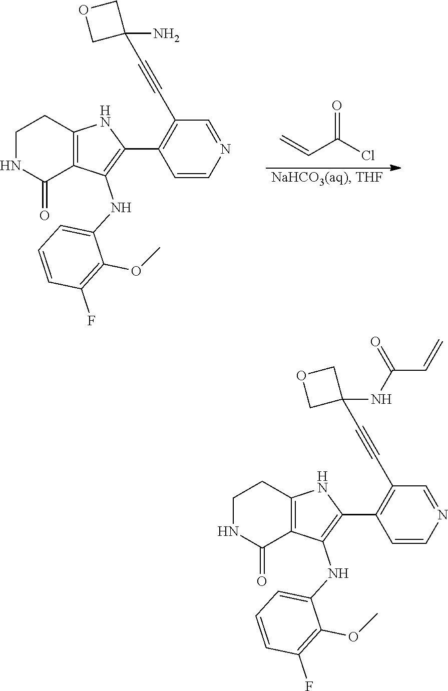

A solution of 2-{3-[2-(3-aminooxetan-3-yl)ethynyl]pyridin-4-yl}-3-[(3-fluoro-2-methoxyphenyl)amino]-1H,5H,6H,7H-pyrrolo[3,2-c]pyridin-4-one (73 mg, 0.16 mmol, 1.00 equiv) in THF (2.00 mL) was basified to pH 8 with NaHCO₃(aq) (2.00 mL). To the above mixture was added acryloyl chloride (16 mg, 0.18 mmol, 1.10 equiv) dropwise at 0° C. under nitrogen atmosphere. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 18% B to 38% B in 9 min, 38% B; Wave Length: 254/220 nm; RT1(min): 9.28; Number Of Runs: 0) to afford N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxetan-3-yl}prop-2-enamide (9.8 mg, 11.8%) as a yellow solid.

LC-MS: [M+H]⁺ found 502.35.

¹H NMR (400 MHz, DMSO-d6) δ 11.27 (s, 1H), 9.45 (s, 1H), 8.58 (s, 1H), 8.30 (d, 1H), 7.51 (s, 1H), 7.35 (d, 1H), 7.17 (s, 1H), 6.65-6.60 (m, 1H), 6.48-6.45 (m, 1H), 6.32-6.18 (m, 2H), 5.99-5.97 (m, 1H), 5.76-5.73 (m, 1H), 4.90 (d, 2H), 4.73 (d, 2H), 3.89 (s, 3H), 3.43-3.39 (m, 2H), 3.02-2.92 (m, 2H).

### Example 152. 3-{[2-(difluoromethoxy)-3-fluorophenyl]amino}-2-(3-{[(2S)-1-(prop-2-enoyl)pyrrolidin-2-yl]methoxy}pyridin-4-yl)-1H,5H,6H,7H-pyrrolo[3,2-c]pyridin-4-one (compound 280)

### 152.1. Synthesis of tert-butyl (2S)-2-({[4-(3-{[2-(difluoromethoxy)-3-fluorophenyl]amino}-4-oxo-1H,5H,6H,7H-pyrrolo[3,2-c]pyridin-2-yl)pyridin-3-yl]oxy}methyl)pyrrolidine-1-carboxylate

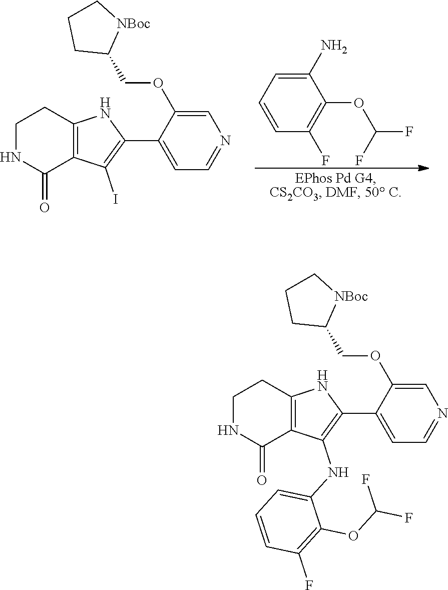

To a stirred mixture of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (100 mg, 0.19 mmol, 1.00 equiv) and 2-(difluoromethoxy)-3-fluoroaniline (49 mg, 0.28 mmol, 1.50 equiv) in DMF (5.00 mL) were added EPhos Pd G4 (17 mg, 0.02 mmol, 0.10 equiv) and Cs₂CO₃ (121 mg, 0.37 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl (2S)-2-({[4-(3-f{[2-(difluoromethoxy)-3-fluorophenyl]amino}-4-oxo-1H,5H,6H,7H-pyrrolo[3,2-c]pyridin-2-yl)pyridin-3-yl]oxy}methyl)pyrrolidine-1-carboxylate (80 mg, 73.30%) as a yellow solid.

LC-MS: (M+H)⁺ found: 588.1.

### 152.2. Synthesis of 3-{[2-(difluoromethoxy)-3-fluorophenyl]amino}-2-{3-[(2S)-pyrrolidin-2-ylmethoxy]pyridin-4-yl}-1H,5H,6H,7H-pyrrolo[3,2-c]pyridin-4-one

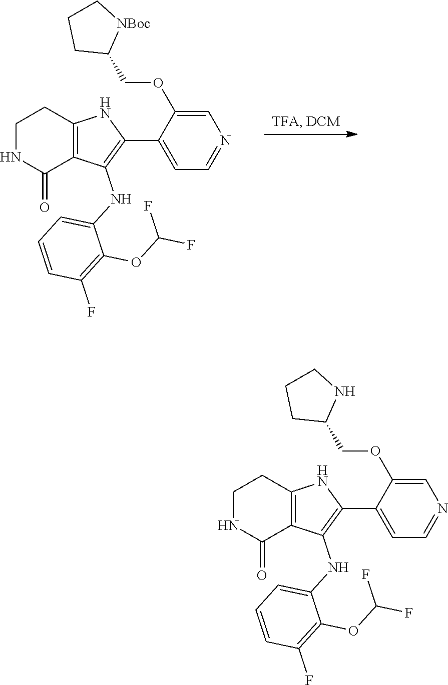

To a stirred mixture of tert-butyl (2S)-2-({[4-(3-{[2-(difluoromethoxy)-3-fluorophenyl]amino}-4-oxo-1H,5H,6H,7H-pyrrolo[3,2-c]pyridin-2-yl)pyridin-3-yl]oxy}methyl)pyrrolidine-1-carboxylate (60 mg, 0.04 mmol in DCM (3.00 mL) was added TFA (1.00 mL) at room temperature under nitrogen atmosphere. The resulting mixture was stirred for 1 h and concentrated under reduced pressure to afford 3-{[2-(difluoromethoxy)-3-fluorophenyl]amino}-2-{3-[(2S)-pyrrolidin-2-ylmethoxy]pyridin-4-yl}-1H,5H,6H,7H-pyrrolo[3,2-c]pyridin-4-one (48 mg, crude) as a red oil.

LC-MS: (M+H)⁺ found: 488.2

### 152.3. Synthesis of 3-{[2-(difluoromethoxy)-3-fluorophenyl]amino}-2-(3-{[(2S)-1-(prop-2-enoyl)pyrrolidin-2-yl]methoxy}pyridin-4-yl)-1H,5H,6H,7H-pyrrolo[3,2-c]pyridin-4-one

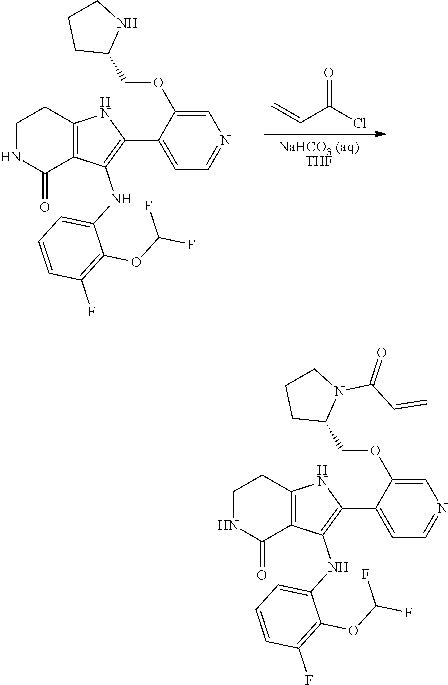

A solution of 3-{[2-(difluoromethoxy)-3-fluorophenyl]amino}-2-{3-[(2S)-pyrrolidin-2-ylmethoxy]pyridin-4-yl}-1H,5H,6H,7H-pyrrolo[3,2-c]pyridin-4-one (48 mg, 0.10 mmol, 1.00 equiv) in THF (1.5 mL) was basified to pH 8 with NaHCO₃(2.00 mL). To the above mixture was added acryloyl chloride (9 mg, 0.09 mmol, 1.00 equiv) at 0° C. under nitrogen atmosphere. The resulting mixture was stirred for 1 h at room temperature under nitrogen atmosphere. The resulting mixture was concentrated under reduced pressure. The residue was purified by silica gel column chromatography, eluted with CH₂Cl₂/MeOH (20:1) to afford the crude product (40 mg). The crude product (40 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 24% B to 48% B in 8 min, 48% B; Wave Length: 254/220 nm; RT1(min): 8; Number Of Runs: 0) to afford 3-{[2-(difluoromethoxy)-3-fluorophenyl]amino}-2-(3-{[(2S)-1-(prop-2-enoyl)pyrrolidin-2-yl]methoxy}pyridin-4-yl)-1H,5H,6H,7H-pyrrolo[3,2-c]pyridin-4-one (19.7 mg, 36.69%) as a yellow solid.

LC-MS: (M+H)⁺ found: 542.1.

¹H NMR (400 MHz, Chloroform-d) δ 11.58 (s, 1H), 8.24 (s, 1H), 8.00 (d, 1H), 7.59 (d, 1H), 7.31 (s, 1H), 7.01-6.73 (m, 2H), 6.64-6.37 (m, 3H), 6.26-6.19 (m, 1H), 5.85-5.78 (m, 1H), 5.28 (s, 1H), 5.12-5.00 (m, 1H), 4.28 (t, 1H), 4.15-4.08 (m, 1H), 3.78 (t, 2H), 3.71-3.56 (m, 2H), 3.28-3.18 (m, 2H), 2.26-2.10 (m, 3H), 1.93-1.90 (m, 1H).

### Example 153. 3-[(3-chloro-2-fluorophenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (compound 279)

### 153.1. Synthesis of tert-butyl (2S)-2-{[(4-{3-[(3-chloro-2-fluorophenyl)amino]-4-oxo-1H,5H,6H,7H-pyrrolo[3,2-c]pyridin-2-yl}pyridin-3-yl)oxy]methyl}pyrrolidine-1-carboxylate

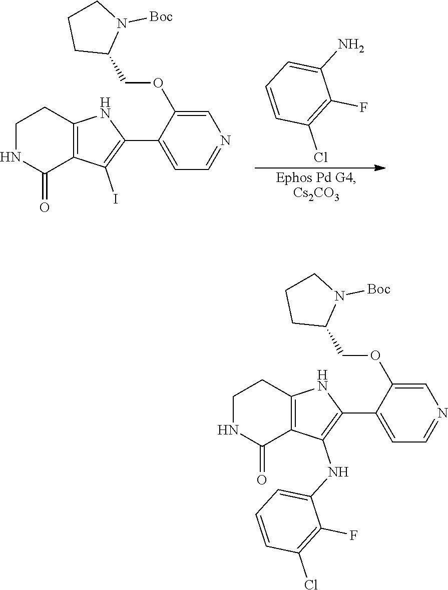

To a stirred mixture of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (100 mg, 0.19 mmol, 1.00 equiv) and 3-chloro-2-fluoroaniline (27 mg, 0.19 mmol, 1.00 equiv) in DMF (2.00 mL) were added EPhos Pd G4 (17 mg, 0.02 mmol, 0.10 equiv) and Cs₂CO₃ (182 mg, 0.56 mmol, 3.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl (2S)-2-{[(4-{3-[(3-chloro-2-fluorophenyl)amino]-4-oxo-1H,5H,6H,7H-pyrrolo[3,2-c]pyridin-2-yl}pyridin-3-yl)oxy]methyl}pyrrolidine-1-carboxylate (73 mg, 70.68%) as a yellow solid.

LC-MS: (M+H)⁺ found: 556.2.

### 153.2. Synthesis of 3-[(3-chloro-2-fluorophenyl)amino]-2-{3-[(2S)-pyrrolidin-2-ylmethoxy]pyridin-4-yl}-1H,5H,6H,7H-pyrrolo[3,2-c]pyridin-4-one

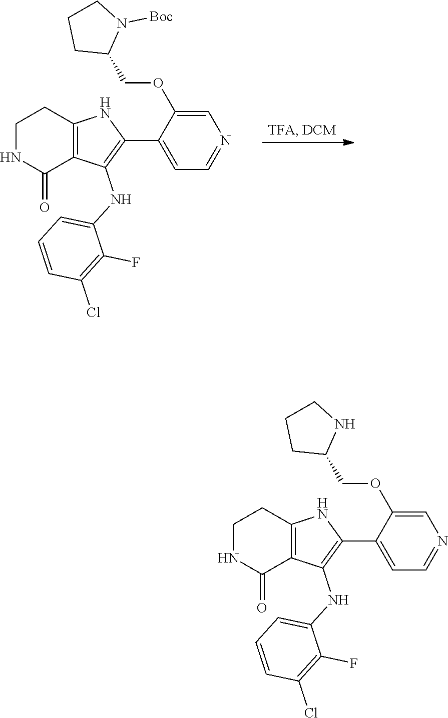

To a stirred mixture of tert-butyl (2S)-2-{[(4-{3-[(3-chloro-2-fluorophenyl)amino]-4-oxo-1H,5H,6H,7H-pyrrolo[3,2-c]pyridin-2-yl}pyridin-3-yl)oxy]methyl}pyrrolidine-1-carboxylate (73 mg, 0.13 mmol) in DCM (3.00 mL) was added TFA (1.50 mL) at 0° C. under nitrogen atmosphere. The resulting mixture was stirred for 1 h and concentrated under reduced pressure to afford 3-[(3-chloro-2-fluorophenyl)amino]-2-{3-[(2S)-pyrrolidin-2-ylmethoxy]pyridin-4-yl}-1H,5H,6H,7H-pyrrolo[3,2-c]pyridin-4-one (59 mg, crude) as a red oil.

LC-MS: (M+H)⁺ found: 456.10.

### 153.3. Synthesis of 3-[(3-chloro-2-fluorophenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one

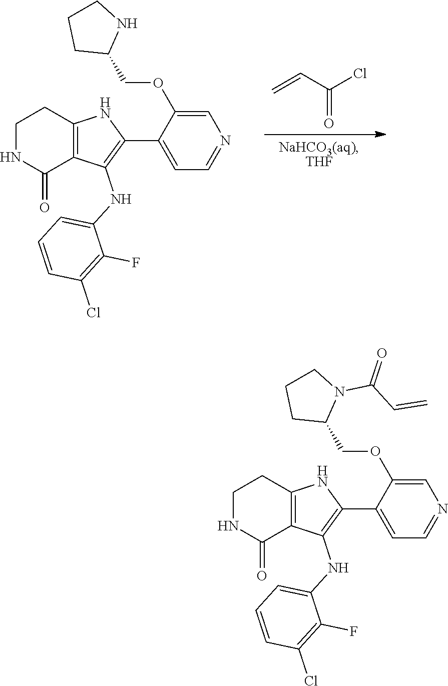

A solution of 3-[(3-chloro-2-fluorophenyl)amino]-2-{3-[(2S)-pyrrolidin-2-ylmethoxy]pyridin-4-yl}-1H,5H,6H,7H-pyrrolo[3,2-c]pyridin-4-one (59 mg, 0.13 mmol, 1.00 equiv) in THF (1.50 mL) was basified to pH 8 with NaHCO₃(aq) (1.5 mL). Acryloyl chloride (12 mg, 0.13 mmol, 1.00 equiv) was added dropwise at 0° C. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product (70 mg) that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 25% B to 43% B in 9 min, 43% B; Wave Length: 254/220 nm; RT1(min): 9.67; Number Of Runs: 0) to afford 3-[(3-chloro-2-fluorophenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (14.2 mg, 20.98%) as a light yellow solid.

LC-MS: (M+H)⁺ found: 510.10.

¹H NMR (400 MHz, Chloroform-d): δ 11.63 (s, 1H), 8.21 (s, 1H), 7.93 (d, 1H), 7.52-7.47 (m, 2H), 6.78 (t, 1H), 6.65-6.54 (m, 2H), 6.42-6.37 (m, 1H), 6.28-6.24 (m, 1H), 5.81-5.78 (m, 1H), 5.32 (s, 1H), 5.09-5.04 (m, 1H), 4.27 (t, 1H), 4.12-4.09 (m, 1H), 3.78-3.74 (m, 2H), 3.64-3.61 (m, 2H), 3.25-3.21 (m, 2H), 2.17-2.13 (m, 3H), 1.95-1.83 (m, 1H).

### Example 154. 3-[(2-chloro-3-fluorophenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (compound 278)

### 154.1. Synthesis of tert-butyl (2S)-2-{[(4-{3-[(2-chloro-3-fluorophenyl)amino]-4-oxo-1H,5H,6H,7H-pyrrolo[3,2-c]pyridin-2-yl}pyridin-3-yl)oxy]methyl}pyrrolidine-1-carboxylate

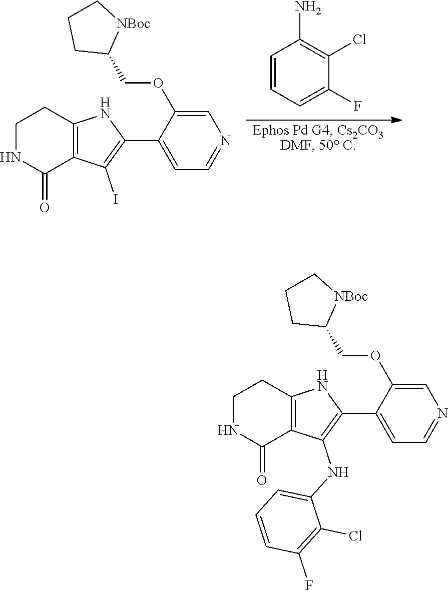

To a stirred solution of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (150 mg, 0.27 mmol, 1.00 equiv) and 2-chloro-3-fluoroaniline (40 mg, 0.27 mmol, 1.00 equiv) in DMF (1.50 mL) was added EPhos Pd G4 (51 mg, 0.056 mmol, 0.20 equiv) and Cs₂CO₃ (181 mg, 0.55 mmol, 2.00 equiv). The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (10:1) to afford tert-butyl (2S)-2-{[(4-{3-[(2-chloro-3-fluorophenyl)amino]-4-oxo-1H,5H,6H,7H-pyrrolo[3,2-c]pyridin-2-yl}pyridin-3-yl)oxy]methyl}pyrrolidine-1-carboxylate (75 mg, 48.41%) as a yellow oil.

LC-MS: (M+H)⁺ found: 556.10.

### 154.2. Synthesis of 3-[(2-chloro-3-fluorophenyl)amino]-2-{3-[(2S)-pyrrolidin-2-ylmethoxy]pyridin-4-yl}-1H,5H,6H,7H-pyrrolo[3,2-c]pyridin-4-one

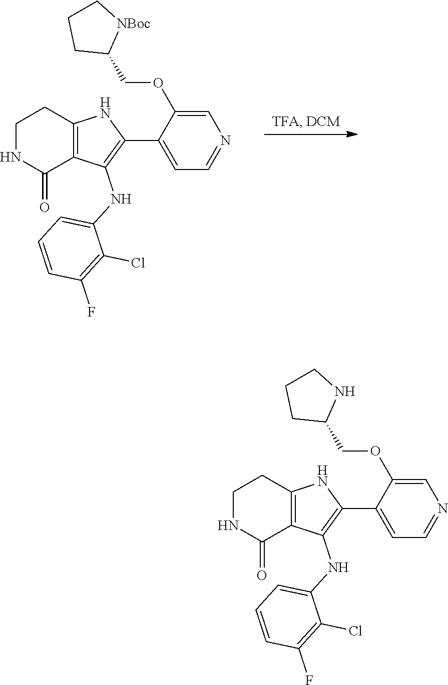

To a stirred solution of tert-butyl (2S)-2-{[(4-{3-[(2-chloro-3-fluorophenyl)amino]-4-oxo-1H,5H,6H,7H-pyrrolo[3,2-c]pyridin-2-yl}pyridin-3-yl)oxy]methyl}pyrrolidine-1-carboxylate (55 mg, 0.10 mmol) in DCM (3.00 mL) was added TFA (1.50 mL) dropwise at 0° C. The resulting mixture was stirred for 2 h and concentrated under reduced pressure to afford 3-[(2-chloro-3-fluorophenyl)amino]-2-{3-[(2S)-pyrrolidin-2-ylmethoxy]pyridin-4-yl}-1H,5H,6H,7H-pyrrolo[3,2-c]pyridin-4-one (45 mg, crude) as a red oil.

LC-MS: (M+H)⁺ found: 456.00.

### 154.3. Synthesis of 3-[(2-chloro-3-fluorophenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one

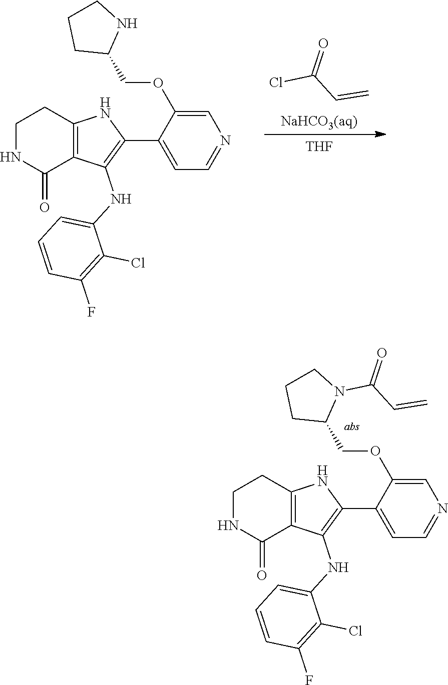

To a stirred solution of 3-[(2-chloro-3-fluorophenyl)amino]-2-{3-[(2S)-pyrrolidin-2-ylmethoxy]pyridin-4-yl}-1H,5H,6H,7H-pyrrolo[3,2-c]pyridin-4-one (45 mg, 0.10 mmol, 1.00 equiv) and NaHCO₃(sat.) (1.00 mL) in THF (1.00 mL) was added acryloyl chloride (10 mg, 0.11 mmol, 1.20 equiv) dropwise at 0° C. The resulting mixture was extracted with EtOAc (3×5 mL). The combined organic layers were washed with brine (1×10 mL), dried over anhydrous Na₂SO₄. After filtration, the filtrate was concentrated under reduced pressure. The crude product (50 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 20% B to 45% B in 9 min, 45% B; Wave Length: 254/220 nm; RT1(min): 9.67; Number Of Runs: 0) to afford 3-[(2-chloro-3-fluorophenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (9.1 mg, 17.90%) as a yellow solid.

LC-MS: (M+H)⁺ found: 510.10.

¹H NMR (300 MHz, Chloroform-d) δ 11.60 (s, 1H), 8.22 (s, 1H), 7.94 (d, 1H), 7.71 (s, 1H), 7.42 (d, 1H), 6.82-6.75 (m, 1H), 6.60-6.51 (m, 2H), 6.43-6.36 (m, 1H), 6.21-6.17 (m, 1H), 5.81-5.77 (m, 1H), 5.26 (s, 1H), 5.04 (t, 1H), 4.26 (t, 1H), 4.13-4.09 (m, 1H), 3.76-3.74 (m, 2H), 3.63-3.60 (m, 2H), 3.25-3.20 (m, 2H), 2.19-2.12 (m, 3H), 2.09-1.87 (m, 1H).

### Example 155. 3-[(3-chloro-2-methylphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (compound 276)

### 155.1. Synthesis of tert-butyl (2S)-2-{[(4-{3-[(3-chloro-2-methylphenyl)amino]-4-oxo-1H,5H,6H,7H-pyrrolo[3,2-c]pyridin-2-yl}pyridin-3-yl)oxy]methyl}pyrrolidine-1-carboxylate

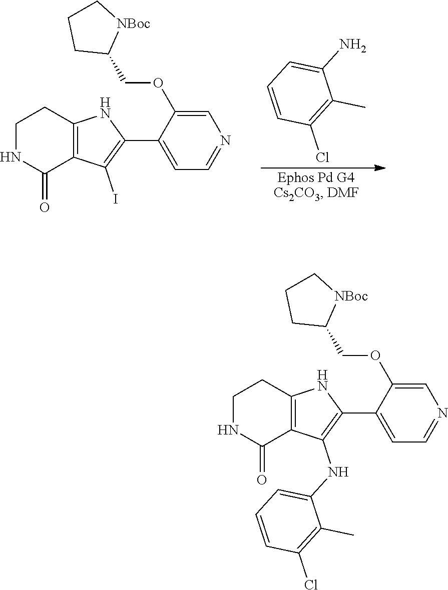

To a stirred mixture of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (100 mg, 0.18 mmol, 1.00 equiv) and 2-methyl-3-chloroaniline (26 mg, 0.18 mmol, 1.00 equiv) in DMF (2.00 mL) were added Cs₂CO₃ (182 mg, 0.55 mmol, 3.00 equiv) and Ephos Pd G4 (17 mg, 0.02 mmol, 0.10 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (10:1) to afford tert-butyl (2S)-2-{[(4-{3-[(3-chloro-2-methylphenyl)amino]-4-oxo-1H,5H,6H,7H-pyrrolo[3,2-c]pyridin-2-yl}pyridin-3-yl)oxy]methyl}pyrrolidine-1-carboxylate (75 mg, 73.14%) as a yellow solid.

LC-MS: [M+H]+ found 552.07.

### 155.2. Synthesis of 3-[(3-chloro-2-methylphenyl)amino]-2-{3-[(2S)-pyrrolidin-2-ylmethoxy]pyridin-4-yl}-1H,5H,6H,7H-pyrrolo[3,2-c]pyridin-4-one

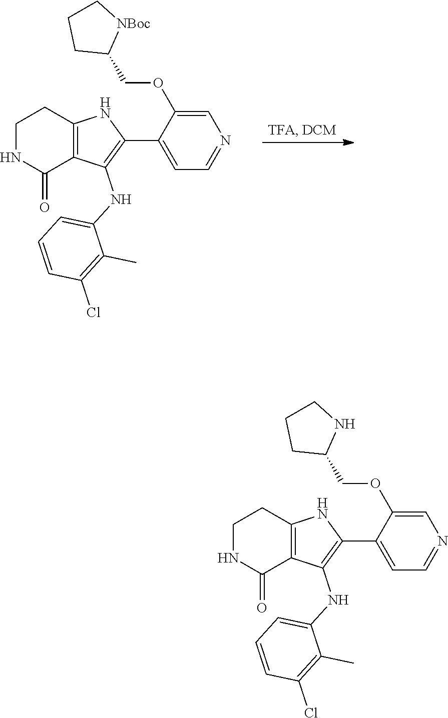

To a stirred mixture of tert-butyl (2S)-2-{[(4-{3-[(3-chloro-2-methylphenyl)amino]-4-oxo-1H,5H,6H,7H-pyrrolo[3,2-c]pyridin-2-yl}pyridin-3-yl)oxy]methyl}pyrrolidine-1-carboxylate (20 mg, 0.04 mmol) in DCM (2.00 mL) was added TFA (1.00 mL) at room temperature under nitrogen atmosphere. The resulting mixture was stirred for 3 h and concentrated under reduced pressure to afford 3-[(3-chloro-2-methylphenyl)amino]-2-{3-[(2S)-pyrrolidin-2-ylmethoxy]pyridin-4-yl}-1H,5H,6H,7H-pyrrolo[3,2-c]pyridin-4-one (49 mg, crude) as a red oil.

LC-MS: [M+H]⁺ found 452.2.

### 155.3. Synthesis of 3-[(3-chloro-2-methylphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one

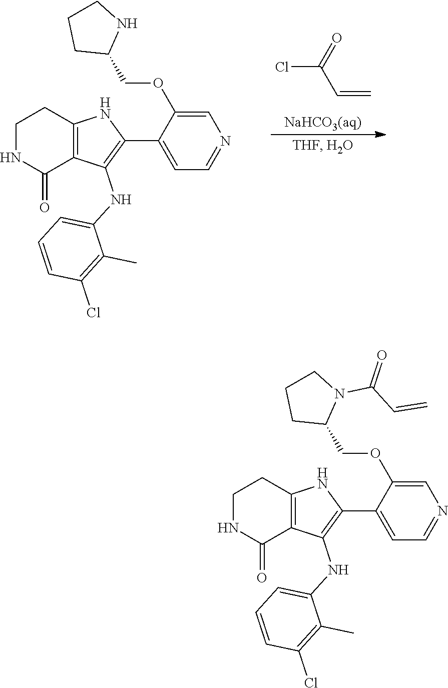

To a stirred solution of 3-[(3-chloro-2-methylphenyl)amino]-2-{3-[(2S)-pyrrolidin-2-ylmethoxy]pyridin-4-yl}-1H,5H,6H,7H-pyrrolo[3,2-c]pyridin-4-one (49 mg, 0.11 mmol, 1.00 equiv) in tetrahydrofuran (2.00 mL) and NaHCO₃(aq.) (2.00 mL) was added acryloyl chloride (10 mg, 0.12 mmol, 1.10 equiv) dropwise at 0° C. under air atmosphere. The resulting mixture was stirred for 2 h at room temperature. The resulting mixture was extracted with DCM (3×10 mL), dried over anhydrous Na₂SO₄. After filtration, the filtrate was concentrated under reduced pressure. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 33% B to 55% B in 8 min, 55% B; Wave Length: 254/220 nm; RT1(min): 6.32; Number Of Runs: 0) to afford 3-[(3-chloro-2-methylphenyl)amino]-2-(3-{[(2S)-1-(prop-2-enoyl)pyrrolidin-2-yl]methoxy}pyridin-4-yl)-1H,5H,6H,7H-pyrrolo[3,2-c]pyridin-4-one (12.8 mg, 22.31%) as a yellow solid.

LC-MS: [M+H]⁺ found 506.10.

¹H NMR (400 MHz, Chloroform-d) δ 11.46 (s, 1H), 8.18 (s, 1H), 7.93 (d, 1H), 7.39 (s, 1H), 7.31 (d, 1H), 6.81-6.79 (m, 1H), 6.70 (t, 1H), 6.58-6.51 (m, 1H), 6.42-6.37 (m, 1H), 6.31-6.29 (d, 1H), 5.79-5.76 (m, 1H), 5.22 (s, 1H), 5.04 (t, 1H), 4.25 (t, 1H), 4.11-4.08 (m, 1H), 3.75 (t, 2H), 3.62-3.58 (m, 2H), 3.23-3.19 (m, 2H), 2.51 (s, 3H), 2.17-2.12 (m, 3H), 1.87-1.81 (m, 1H).

### Example 156. 2-(3-{2-[(2R)-1-(but-2-ynoyl)-2-methylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one (compound 291)

### 156.1. Synthesis of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate

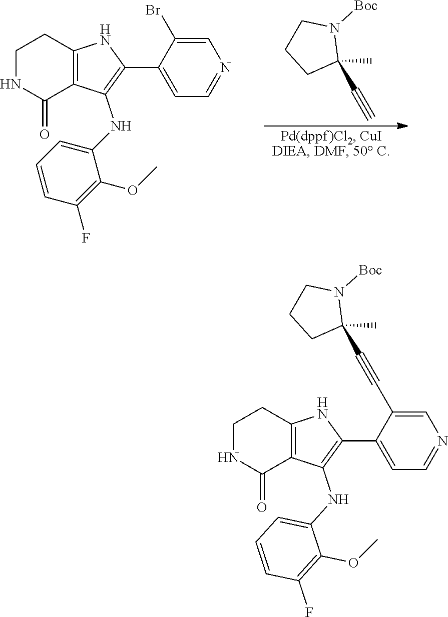 [SMILES: COc1c(F)cccc1Nc1c(-c2ccncc2Br)[nH]c2c1C(=O)NCC2 ; C#C[C@@]1(C)CCCN1C(=O)OC(C)(C)C ; "Pd(dppf)Cl2, CuIDIEA, DMF, 50° C." ; COc1c(F)cccc1Nc1c(-c2ccncc2C#C[C@@]2(C)CCCN2C(=O)OC(C)(C)C)[nH]c2c1C(=O)NCC2]

To a stirred mixture of 2-(3-bromopyridin-4-yl)-3-[(3-fluoro-2-methoxyphenyl)amino]-1H,5H,6H,7H-pyrrolo[3,2-c]pyridin-4-one (300 mg, 0.69 mmol, 1.00 equiv) and tert-butyl (2R)-2-ethynyl-2-methylpyrrolidine-1-carboxylate (509 mg, 2.43 mmol, 3.50 equiv) in DMF (5.00 mL) were added Pd(dppf)C12 DCM (142 mg, 0.17 mmol, 0.25 equiv) and CuI (66 mg, 0.35 mmol, 0.50 equiv) at room temperature under argon atmosphere. The resulting mixture was stirred for overnight at 50° C. under argon atmosphere. The residue was purified by reverse flash chromatography with the following conditions (column, silica gel; mobile phase, MeCN in water, 10% to 80% gradient in 10 min; detector, UV 254 nm) to afford tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (270 mg, crude). Then the residue was purified by silica gel column chromatography, eluted with DCM/MeOH (10:1) to afford tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (250 mg, 64.22%) as a yellow solid.

LC-MS: [M+H]+ found 560.20. 156.2. Synthesis of 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one

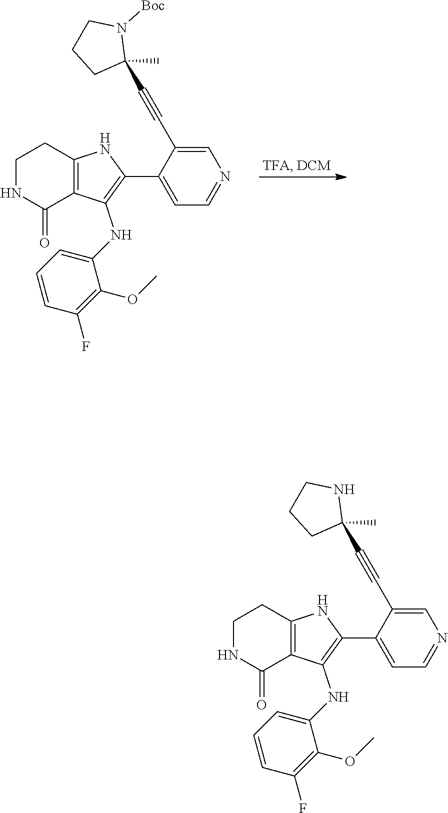 [SMILES: COc1c(F)cccc1Nc1c(-c2ccncc2C#C[C@@]2(C)CCCN2C(=O)OC(C)(C)C)[nH]c2c1C(=O)NCC2 ; COc1c(F)cccc1Nc1c(-c2ccncc2C#C[C@@]2(C)CCCN2)[nH]c2c1C(=O)NCC2]

To a stirred mixture of tert-butyl (2R)-2-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-methylpyrrolidine-1-carboxylate (80 mg, 0.14 mmol, 1.00 equiv) in DCM (1.50 mL) was added TFA (0.50 mL) at room temperature under nitrogen atmosphere. The resulting mixture was stirred for 1 h and concentrated under reduced pressure to afford 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (80 mg, crude) as a red oil.

LC-MS: [M+H]+ found 460.10.

### 156.3. Synthesis of 2-(3-{2-[(2R)-1-(but-2-ynoyl)-2-methylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one

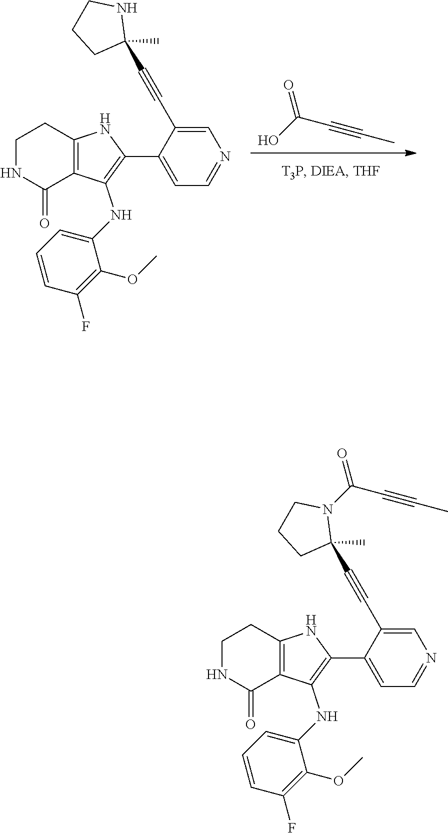

A solution of 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (65 mg, 0.14 mmol, 1.00 equiv) in THF (1.00 mL) was basified to pH 8 with DIEA. To the above mixture was added 2-butynoic acid (18 mg, 0.21 mmol, 1.50 equiv) at 0° C. under nitrogen atmosphere followed by the addition of T₃P (90 mg, 0.28 mmol, 2.00 equiv, 50% in EA) dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product (80 mg) that was purified by Prep-HPLC under the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 21% B to 47% B in 8 min, 47% B; Wave Length: 254/220 nm; RT1(min): 8; Number Of Runs: 0) to afford 2-(3-{2-[(2R)-1-(but-2-ynoyl)-2-methylpyrrolidin-2-yl]ethynyl}pyridin-4-yl)-3-[(3-fluoro-2-methoxyphenyl)amino]-1H,5H,6H,7H-pyrrolo[3,2-c]pyridin-4-one (27.8 mg, 37.06%) as a yellow solid.

LC-MS: [M+H]⁺ found 526.10.

¹H NMR (400 MHz, Chloroform-d) δ 11.48 (s, 1H), 8.70 (s, 1H), 8.15 (s, 1H), 7.97 (d, J=6.4 Hz, 1H), 7.49 (d, J=6.4 Hz, 1H), 6.66-6.57 (m, 2H), 6.24 (s, 1H), 6.02 (d, J=7.2 Hz, 1H), 4.10 (d, J=1.6 Hz, 3H), 3.94-3.79 (m, 2H), 3.66 (t, J=6.4 Hz, 2H), 3.43-3.35 (m, 1H), 3.22-3.15 (m, 1H), 2.58-2.52 (m, 1H), 2.24-2.07 (m, 3H), 2.04 (s, 3H), 1.74 (s, 3H).

### Example 157. 2-(3-{[(2S)-1-(but-2-ynoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (compound 290)

### 157.1. Synthesis of tert-butyl (2S)-2-{[(4-bromopyridin-3-yl)oxy]methyl}azetidine-1-carboxylate

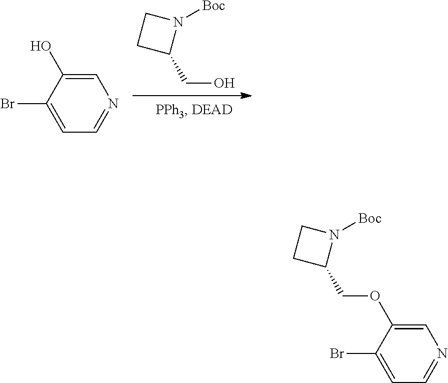 [SMILES: Oc1cnccc1Br ; CC(C)(C)OC(=O)N1CC[C@H]1CO ; CC(C)(C)OC(=O)N1CC[C@H]1COc1cnccc1Br]

To a stirred solution of 4-bromopyridin-3-ol (1.5 g, 8.621 mmol, 1 equiv) in THF (50 mL) was added PPh₃ (3.39 g, 12.93 mmol, 1.50 equiv) and tert-butyl (2S)-2-(hydroxymethyl)azetidine-1-carboxylate (1.78 g, 9.48 mmol, 1.10 equiv) at 0° C., the mixture was stirred for 10 min at 0° C. under nitrogen atmosphere. DEAD (2.25 g, 12.93 mmol, 1.50 equiv) was added dropwise at 0° C. The mixture was stirred for 1.5 h at 0° C. under nitrogen atmosphere. The resulting mixture was concentrated under reduced pressure. The residue was purified by reverse flash chromatography with the following conditions (column, C18 gel; mobile phase, MeCN in water, 10% to 60% gradient in 20 min; detector, UV 254 nm) to afford tert-butyl (2S)-2-{[(4-bromopyridin-3-yl)oxy]methyl}azetidine-1-carboxylate (2.57 g, 86.86%) as a yellow oil.

LC-MS: (M+H)⁺ found: 344.95.

### 157.2. Synthesis of tert-butyl (2S)-2-{[(4-{4-oxo-1H,5H,6H,7H-pyrrolo[3,2-c]pyridin-2-yl}pyridin-3-yl)oxy]methyl}azetidine-1-carboxylate

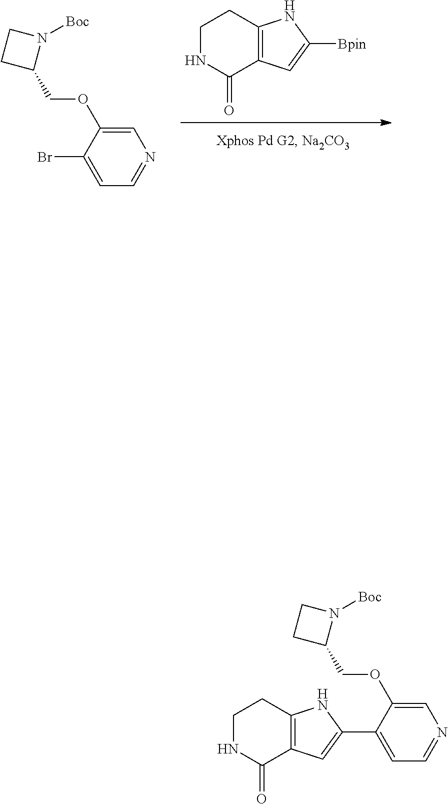

To a stirred solution of tert-butyl (2S)-2-{[(4-bromopyridin-3-yl)oxy]methyl}azetidine-1-carboxylate (500 mg, 1.46 mmol, 1.00 equiv) in 1,4-dioxane (10.00 mL) was added XPhos Pd G2 (115 mg, 0.15 mmol, 0.10 equiv), 2-(4,4,5,5-tetramethyl-1,3,2-dioxaborolan-2-yl)-1H,5H,6H,7H-pyrrolo[3,2-c]pyridin-4-one (458 mg, 1.74 mmol, 1.20 equiv) and Na₂CO₃ (464 mg, 4.37 mmol, 3.00 equiv) in H₂O (2.00 mL) at room temperature. The mixture was stirred for 2 h at 50° C. under argon atmosphere. The resulting mixture was concentrated under reduced pressure. The residue was purified by reverse flash chromatography with the following conditions (column, C18 gel; mobile phase, MeCN in water, 10% to 60% gradient in 15 min; detector, UV 254 nm) to afford tert-butyl (2S)-2-{[(4-{4-oxo-1H,5H,6H,7H-pyrrolo[3,2-c]pyridin-2-yl}pyridin-3-yl)oxy]methyl}azetidine-1-carboxylate (0.56 g, 96.22%) as a brown yellow solid.

LC-MS: (M+H)⁺ found: 399.05.

### 157.3. Synthesis of tert-butyl (2S)-2-{[(4-{3-iodo-4-oxo-1H,5H,6H,7H-pyrrolo[3,2-c]pyridin-2-yl}pyridin-3-yl)oxy]methyl}azetidine-1-carboxylate

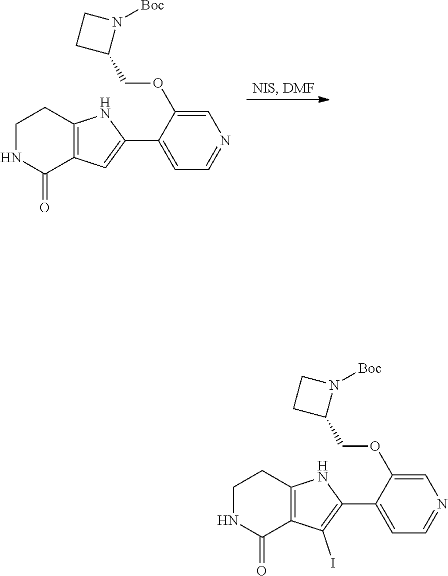 [SMILES: CC(C)(C)OC(=O)N1CC[C@H]1COc1cnccc1-c1cc2c([nH]1)CCNC2=O ; CC(C)(C)OC(=O)N1CC[C@H]1COc1cnccc1-c1[nH]c2c(c1I)C(=O)NCC2]

To a stirred solution of tert-butyl (2S)-2-{[(4-{4-oxo-1H,5H,6H,7H-pyrrolo[3,2-c]pyridin-2-yl}pyridin-3-yl)oxy]methyl}azetidine-1-carboxylate (560 mg, 1.41 mmol, 1.00 equiv) in DMF (7.00 mL) was added NIS (316 mg, 1.41 mmol, 1.00 equiv) dropwise at 0° C. The mixture was stirred for 1 h at room temperature under nitrogen atmosphere. The reaction was quenched with 1 mL sat. Na₂SO₃ (aq.) at 0° C. The residue was purified by reverse flash chromatography with the following conditions (column, C18 gel; mobile phase, MeCN in water, 10% to 60% gradient in 15 min; detector, UV 254 nm) to afford tert-butyl (2S)-2-{[(4-{3-iodo-4-oxo-1H,5H,6H,7H-pyrrolo[3,2-c]pyridin-2-yl}pyridin-3-yl)oxy]methyl}azetidine-1-carboxylate (550 mg, 74.52%) as a yellow solid.

LC-MS: (M+H)⁺ found: 524.95.

### 157.4. Synthesis of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate

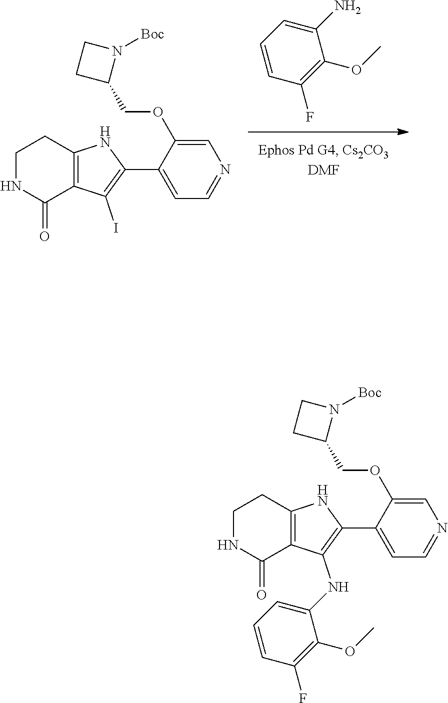

To a stirred solution of tert-butyl (2S)-2-{[(4-{3-iodo-4-oxo-1H,5H,6H,7H-pyrrolo[3,2-c]pyridin-2-yl}pyridin-3-yl)oxy]methyl}azetidine-1-carboxylate (500 mg, 0.954 mmol, 1.00 equiv) in DMF (5.00 mL) were added EPhos Pd G4 (87.59 mg, 0.095 mmol, 0.10 equiv), Cs₂CO₃ (932 mg, 2.86 mmol, 3.00 equiv) and 3-fluoro-2-methoxyaniline (403 mg, 2.86 mmol, 3.00 equiv) at room temperature. The mixture was stirred for 2 h at 50° C. under argon atmosphere. The resulting mixture was concentrated under reduced pressure. The residue was purified by silica gel column chromatography, eluted with CH₂Cl₂/MeOH (96:4) to afford tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (500 mg, 97.66%) as a yellow solid.

LC-MS: (M+H)⁺ found: 538.30.

### 157.5. Synthesis of 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one

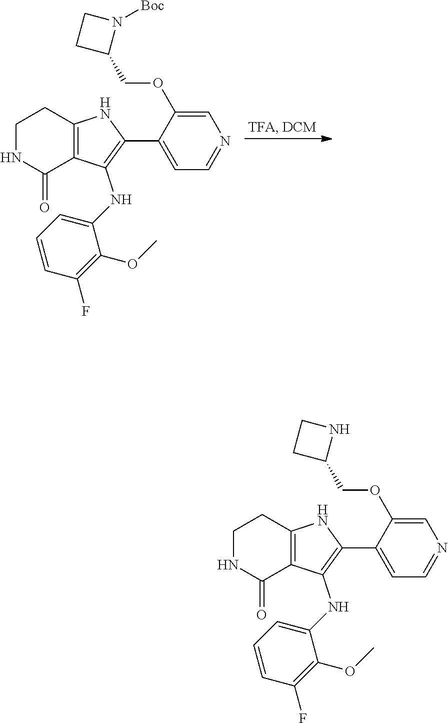 [SMILES: COc1c(F)cccc1Nc1c(-c2ccncc2OC[C@@H]2CCN2C(=O)OC(C)(C)C)[nH]c2c1C(=O)NCC2 ; COc1c(F)cccc1Nc1c(-c2ccncc2OC[C@@H]2CCN2)[nH]c2c1C(=O)NCC2]

To a stirred mixture of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (180 mg, 0.33 mmol) in DCM (4.00 mL) was added TFA (2.00 mL) at 0° C. under nitrogen atmosphere. The resulting mixture was stirred for 1 h and concentrated under reduced pressure to afford 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one (260 mg, crude) as a red oil.

LC-MS: (M+H)⁺ found: 438.05.

### 157.6. Synthesis of 2-(3-{[(2S)-1-(but-2-ynoyl)azetidin-2-yl]methoxy}pyridin-4-yl)-3-[(3-fluoro-2-methoxyphenyl)amino]-1H,5H,6H,7H-pyrrolo[3,2-c]pyridin-4-one

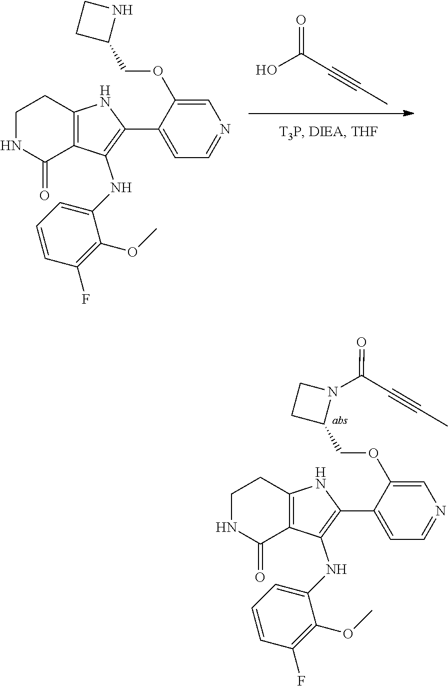

A solution of 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one (260 mg, 0.59 mmol, 1.00 equiv) in THF (5.00 mL) was basified to pH 8 with DIEA. To the above mixture was added 2-butynoic acid (75 mg, 0.89 mmol, 1.50 equiv) at 0° C. under nitrogen atmosphere followed by the addition of T₃P (756 mg, 1.19 mmol, 2.00 equiv, 50% in EA) dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product (280 mg) that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 20% B to 45% B in 10 min, 45% B; Wave Length: 254/220 nm; RT1(min): 8.85; Number Of Runs: 0) to afford 2-(3-{[(2S)-1-(but-2-ynoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (28.0 mg, 9.34%) as a light yellow solid.

LC-MS: (M+H)⁺ found: 504.10.

¹H NMR (400 MHz, CDCl3): δ 11.46 (s, 1H), 8.24 (s, 1H), 7.96 (d, 1H), 7.70 (s, 1H), 7.49 (d, 1H), 6.59-6.51 (m, 1H), 6.49-6.46 (m, 1H), 6.04 (d, 1H), 5.20 (s, 1H), 4.98-4.88 (m, 1H), 4.50 (t, 1H), 4.28-4.24 (m, 3H), 4.10 (s, 3H), 3.60-3.56 (m, 2H), 3.21-3.17 (m, 1H), 3.09-3.05 (m, 1H), 2.69-2.57 (m, 1H), 2.14-2.12 (m, 1H), 2.04 (s, 3H).

### Example 158. 2-(3-{[(2S)-1-(but-2-ynoyl)azetidin-2-yl]methoxy}pyridin-4-yl)-3-[(3-chloro-2-methoxyphenyl)amino]-1H,5H,6H,7H-pyrrolo[3,2-c]pyridin-4-one (compound 288)

### 158.1. Synthesis of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}azetidine-1-carboxylate

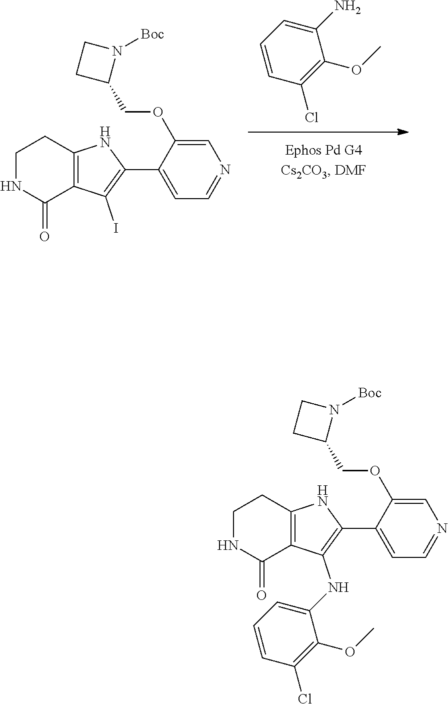

To a stirred mixture of tert-butyl tert-butyl (2S)-2-{[(4-{3-iodo-4-oxo-1H,5H,6H,7H-pyrrolo[3,2-c]pyridin-2-yl}pyridin-3-yl)oxy]methyl}azetidine-1-carboxylate (300 mg, 0.57 mmol, 1.00 equiv) and 3-chloro-2-methoxyaniline (90 mg, 0.57 mmol, 1.00 equiv) in DMF (5.00 mL) were added EPhos Pd G4 (52 mg, 0.05 mmol, 0.10 equiv) and Cs₂CO₃ (372 mg, 1.14 mmol, 2.00 equiv) under argon atmosphere. The resulting suspension was backfilled with argon three times and stirred for 2h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (10:1) to afford tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}azetidine-1-carboxylate (300 mg, 75.71%) as a off-white solid.

LC-MS: (M+H)⁺ found 554.1.

### 158.2. Synthesis of 2-{3-[(2S)-azetidin-2-ylmethoxy]pyridin-4-yl}-3-[(3-chloro-2-methoxyphenyl)amino]-1H,5H,6H,7H-pyrrolo[3,2-c]pyridin-4-one

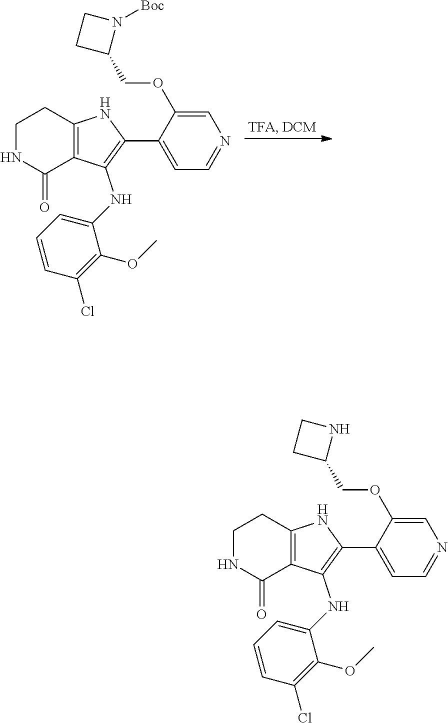 [SMILES: COc1c(Cl)cccc1Nc1c(-c2ccncc2OC[C@@H]2CCN2C(=O)OC(C)(C)C)[nH]c2c1C(=O)NCC2 ; COc1c(Cl)cccc1Nc1c(-c2ccncc2OC[C@@H]2CCN2)[nH]c2c1C(=O)NCC2]

To a stirred mixture of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}azetidine-1-carboxylate (260 mg, 0.46 mmol) in DCM (2.00 mL) was added TFA (2.00 mL) at room temperature under nitrogen atmosphere. The resulting mixture was stirred for 1 h and concentrated under reduced pressure. The residue product was purified by reverse phase flash with the following conditions (Mobile Phase A: Water (10 mmol/L NH₄HCO₃), Mobile Phase B: ACN; Flow rate: 60 mL/min; Gradient: 30% B to 100% B in 45 min;) to afford 2-{3-[(2S)-azetidin-2-ylmethoxy]pyridin-4-yl}-3-[(3-chloro-2-methoxyphenyl)amino]-1H,5H,6H,7H-pyrrolo[3,2-c]pyridin-4-one (110 mg, 51.64%) as an off-white solid.

LC-MS: (M+H)⁺ found 454.15.

### 158.3. Synthesis of 2-(3-{[(2S)-1-(but-2-ynoyl)azetidin-2-yl]methoxy}pyridin-4-yl)-3-[(3-chloro-2-methoxyphenyl)amino]-1H,5H,6H,7H-pyrrolo[3,2-c]pyridin-4-one

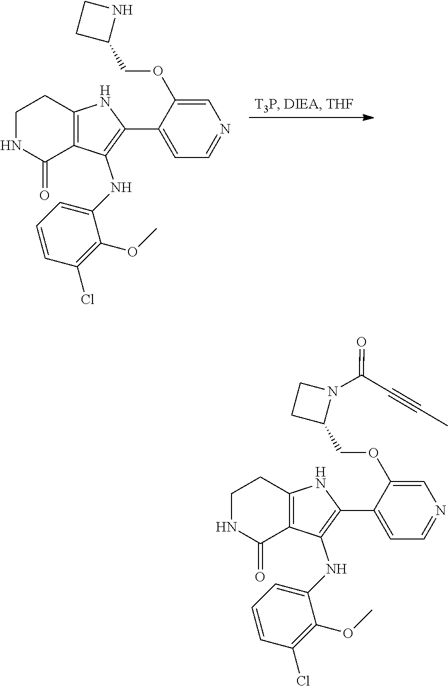 [SMILES: COc1c(Cl)cccc1Nc1c(-c2ccncc2OC[C@@H]2CCN2)[nH]c2c1C(=O)NCC2 ; CC#CC(=O)N1CC[C@H]1COc1cnccc1-c1[nH]c2c(c1Nc1cccc(Cl)c1OC)C(=O)NCC2]

To a stirred solution of 2-{3-[(2S)-azetidin-2-ylmethoxy]pyridin-4-yl}-3-[(3-chloro-2-methoxyphenyl)amino]-1H,5H,6H,7H-pyrrolo[3,2-c]pyridin-4-one (50 mg, 0.11 mmol, 1.00 equiv) in THF (0.50 mL) was added DIEA (0.50 mL) at 0° C. under nitrogen atmosphere. The resulting mixture was stirred for 10 min at room temperature under nitrogen atmosphere. To the above mixture was added 2-butynoic acid (13 mg, 0.16 mmol, 1.50 equiv) and T₃P (140 mg, 0.22 mmol, 2.00 equiv, 50% in EA) at 0° C. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product (70 mg) that was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 32% B to 62% B in 7 min; Wave Length: 254 nm; RT1(min): 6.5; Number Of Runs: 0) to afford 2-(3-{[(2S)-1-(but-2-ynoyl)azetidin-2-yl]methoxy}pyridin-4-yl)-3-[(3-chloro-2-methoxyphenyl)amino]-1H,5H,6H,7H-pyrrolo[3,2-c]pyridin-4-one (28.9 mg, 50.36%) as an off-white solid.

LC-MS: (M+H)⁺ found 520.05.

¹H NMR (300 MHz, Chloroform-d) δ 11.44 (s, 1H), 8.28 (s, 1H), 7.96 (d, J=3.00 Hz, 1H), 7.64 (s, 1H), 7.43 (d, J=3.00 Hz, 1H), 6.73-6.70 (m, 1H), 6.60 (t, J=6.00 Hz, 1H), 6.19-6.16 (m, 1H), 5.33 (s, 1H), 4.94 (d, J=6.00 Hz, 1H), 4.50 (t, J=9 Hz, 1H), 4.29-4.22 (m, 3H), 4.07 (s, 3H), 3.60-3.55 (m, 2H), 3.21-3.03 (m, 2H), 2.65-2.54 (m, 1H), 2.16-2.11 (m, 1H), 2.03 (s, 3H).

### Example 159. 2-(3-{[(2R)-1-(but-2-ynoyl)pyrrolidin-2-yl]methoxy}pyridin-4-yl)-3-[(3-fluoro-2-methoxyphenyl)amino]-1H,5H,6H,7H-pyrrolo[3,2-c]pyridin-4-one 2,2,2-trifluoroacetic acid salt (compound 286)

### 159.1. Synthesis of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate

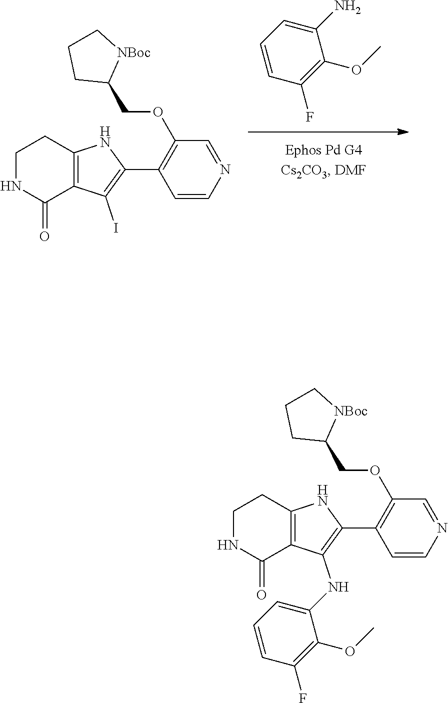

To a stirred mixture of t tert-butyl (2R)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (200 mg, 0.37 mmol, 1.00 equiv) and 3-fluoro-2-methoxyaniline (105 mg, 0.74 mmol, 2.00 equiv) in DMF (5.00 mL) were added Ephos Pd G4 (34 mg, 0.04 mmol, 0.10 equiv) and Cs₂CO₃ (242 mg, 0.74 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (200 mg, 97.60%) as a yellow solid.

LC-MS: [M+H]+ found 552.30.

### 159.2. Synthesis of 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one

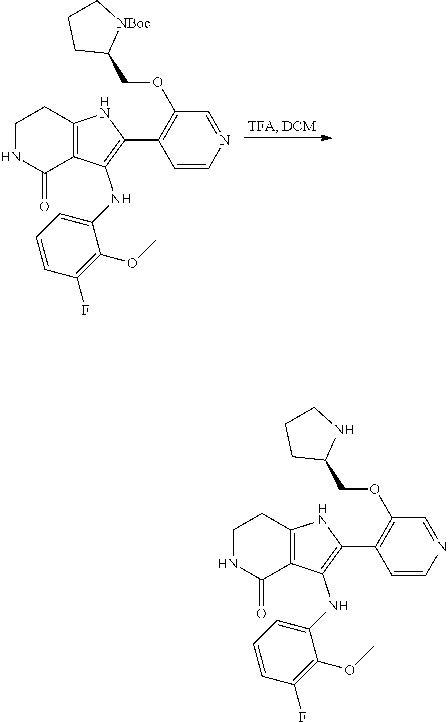 [SMILES: COc1c(F)cccc1Nc1c(-c2ccncc2OC[C@H]2CCCN2C(=O)OC(C)(C)C)[nH]c2c1C(=O)NCC2 ; COc1c(F)cccc1Nc1c(-c2ccncc2OC[C@H]2CCCN2)[nH]c2c1C(=O)NCC2]

To a stirred mixture of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (200 mg, 0.036 mmol) in DCM (1.00 mL) was added TFA (0.50 mL) at room temperature under nitrogen atmosphere. The resulting mixture was stirred for 1 h and concentrated under reduced pressure to afford 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one (120 mg, crude) as a red oil.

LC-MS: [M+H]⁺ found 452.05.

### 159.3. Synthesis of 2-(3-{[(2R)-1-(but-2-ynoyl)pyrrolidin-2-yl]methoxy}pyridin-4-yl)-3-[(3-fluoro-2-methoxyphenyl)amino]-1H,5H,6H,7H-pyrrolo[3,2-c]pyridin-4-one 2,2,2-trifluoroacetic acid salt

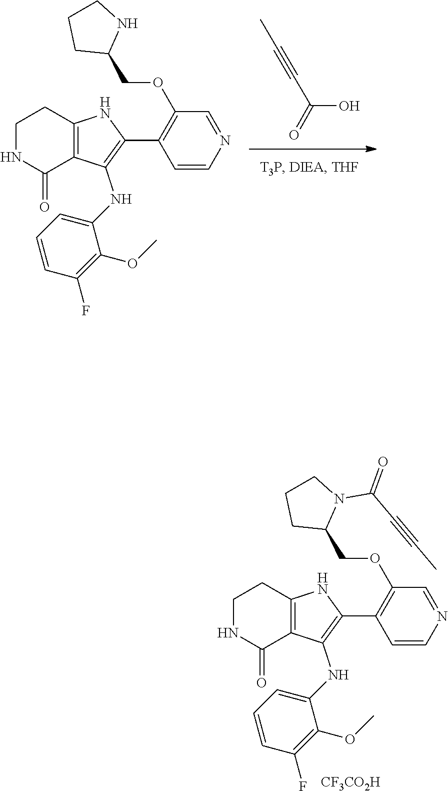

A solution of 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one (77 mg, 0.17 mmol, 1.00 equiv) in THF (2.00 mL) was basified to pH 8 with DIEA. To the above mixture was added 2-butynoic acid (22 mg, 0.26 mmol, 1.50 equiv) at 0° C. under nitrogen atmosphere followed by the addition of T₃P (108 mg, 0.34 mmol, 2.00 equiv, 50% in EA) dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product (100 mg) that was purified by Prep-HPLC under the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 16% B to 36% B in 8 min, 36% B; Wave Length: 254/220 nm; RT1(min): 8; Number Of Runs: 0) to afford 2-(3-{[(2R)-1-(but-2-ynoyl)pyrrolidin-2-yl]methoxy}pyridin-4-yl)-3-[(3-fluoro-2-methoxyphenyl)amino]-1H,5H,6H,7H-pyrrolo[3,2-c]pyridin-4-one 2,2,2-trifluoroacetic acid salt (45.6 mg, 51.61%) as a yellow solid.

LC-MS: [M+H]+ found 518.45.

¹H NMR (400 MHz, Chloroform-d) δ 11.71 (s, 1H), 8.56 (s, 1H), 7.96 (s, 1H), 7.85 (d, 1H), 7.58 (d, J=6.4 Hz, 1H), 6.64-6.56 (m, 2H), 6.36 (s, 1H), 5.97-5.95 (m, 1H), 4.92 (t, 1H), 4.27 (t, J=10 Hz, 1H), 4.16-4.14 (m, 1H), 4.11 (d, 3H), 4.05-3.99 (m, 1H), 3.72-3.63 (m, 3H), 3.33-3.17 (m, 2H), 2.25-2.22 (m, 2H), 2.12-2.08 (m, 1H), 2.05 (s, 3H), 1.91-1.88 (m, 1H).

### Example 160. 2-(3-{[(2S)-1-(but-2-ynoyl)pyrrolidin-2-yl]methoxy}pyridin-4-yl)-3-[(3-chloro-2-methoxyphenyl) amino]-1H,5H,6H,7H-pyrrolo[3,2-c]pyridin-4-one (compound 285)

### 160.1. Synthesis of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl} pyrrolidine-1-carboxylate

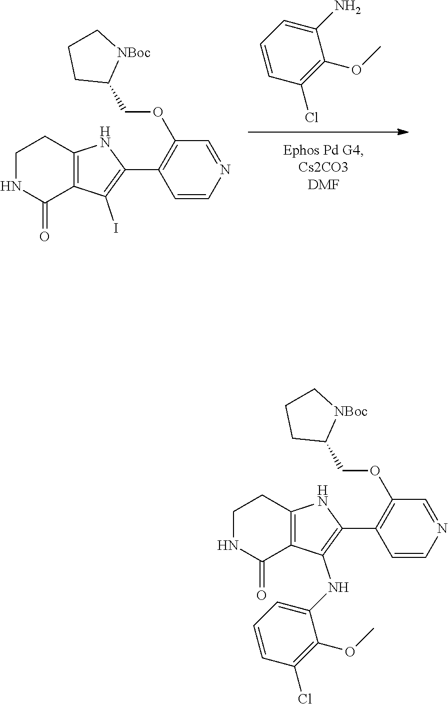

To a stirred solution of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (150 mg, 0.28 mmol, 1.00 equiv) and 3-chloro-2-methoxyaniline (40 mg, 0.25 mmol, 0.90 equiv) in DMF (2.00 mL) were added Cs₂CO₃ (182 mg, 0.56 mmol, 2.00 equiv) and EPhos Pd G4 (26 mg, 0.03 mmol, 0.10 equiv) dropwise at RT under argon atmosphere. The resulting suspension was backfilled with argon three times and stirred for 2 h at 50° C. LCMS confirmed completion of reaction and desired product was observed. The resulting mixture was concentrated under vacuum. The residue was purified by silica gel column chromatography, eluted with MeOH/DCM (97:3) to afford tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl} pyrrolidine-1-carboxylate (107 mg, 64.23%) as a yellow green solid.

LC-MS: (M+H)⁺ found: 567.90.

### 160.2. Synthesis of 3-[(3-chloro-2-methoxyphenyl)amino]-2-{3-[(2S)-pyrrolidin-2-ylmethoxy]pyridin-4-yl}-1H,5H,6H1,71H-pyrrolo[3,2-c]pyridin-4-one

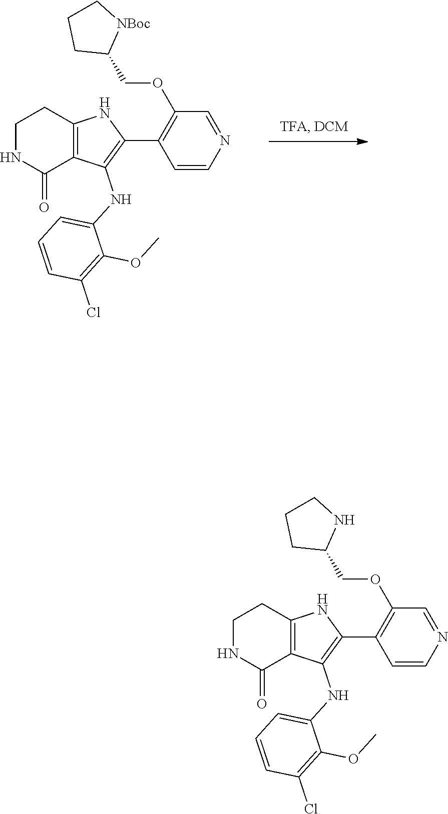 [SMILES: COc1c(Cl)cccc1Nc1c(-c2ccncc2OC[C@@H]2CCCN2C(=O)OC(C)(C)C)[nH]c2c1C(=O)NCC2 ; COc1c(Cl)cccc1Nc1c(-c2ccncc2OC[C@@H]2CCCN2)[nH]c2c1C(=O)NCC2]

To a stirred mixture of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (100 mg, 0.18 mmol, 1.00 equiv) in DCM (1.00 mL) was added TFA (0.50 mL) at room temperature under nitrogen atmosphere. The resulting mixture was stirred for 1 h and concentrated under reduced pressure to afford 3-[(3-chloro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (100 mg, crude) as a red oil.

LC-MS: (M+H)⁺ found: 468.10.

### 160.3. Synthesis of 2-(3-{[(2S)-1-(but-2-ynoyl)pyrrolidin-2-yl]methoxy}pyridin-4-yl)-3-[(3-chloro-2-methoxyphenyl) amino]-1H,5H,6H,7H-pyrrolo[3,2-c]pyridin-4-one

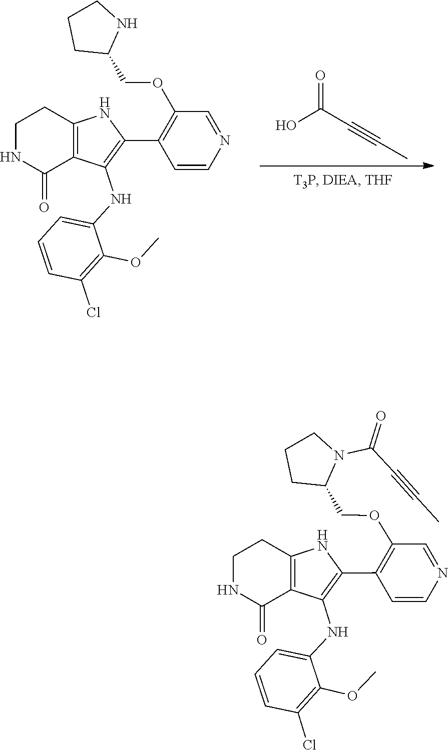

A solution of 3-[(3-chloro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (100 mg, 0.21 mmol, 1.00 equiv) in THF (1.5 mL).was basified to pH 8 with DIEA. To the above mixture was added 2-butynoic acid (27 mg, 0.32 mmol, 1.50 equiv) at 0° C. under nitrogen atmosphere followed by the addition of T₃P (136 mg, 0.43 mmol, 2.00 equiv, 50% in EA) dropwise. The resulting mixture was stirred for 1 h at room temperature under nitrogen atmosphere. Saturated aqueous NaHCO₃(3.00 mL) was added to the reaction mixture at 0° C. and extracted with EtOAc (3×5.00 mL), dried over anhydrous Na₂SO₄ and filtered. The filtrate was concentrated under reduced pressure to give crude product (100 mg) that was purified by Prep-HPLC with the following conditions (Column: XSelect CSH Fluoro Phenyl, 30*150 mm, 5 μm; Mobile Phase A: Water (0.05% TFA), Mobile Phase B: ACN; Flow rate: 60 mL/min; Gradient: 14% B to 44% B in 7 min; Wave Length: 254 nm; RT1(min): 6.5; Number Of Runs: 0) to afford 2-(3-{[(2S)-1-(but-2-ynoyl)pyrrolidin-2-yl]methoxy}pyridin-4-yl)-3-[(3-chloro-2-methoxyphenyl) amino]-1H,5H,6H,7H-pyrrolo[3,2-c]pyridin-4-one (44 mg, 38.48%) as a yellow solid.

LC-MS: (M+H)⁺ found: 534.10.

¹H NMR (400 MHz, Chloroform-d) δ 11.66 (s, 1H), 8.55 (s, 1H), 8.06 (s, 1H), 7.84 (d, 1H), 7.52 (d, 1H), 6.85-6.82 (m, 1H), 6.67 (t, 1H), 6.12-6.10 (m, 1H), 5.78 (s, 1H), 4.93 (t, 1H), 4.29-4.24 (m, 1H), 4.17-4.15 (m, 1H), 4.08-3.99 (m, 4H), 3.72-3.63 (m, 3H), 3.34-3.26 (m, 1H), 3.23-3.15 (m, 1H), 2.28-2.23 (m, 1H), 2.14-2.01 (m, 5H), 1.88 (s, 1H)

### Example 161. 2-(3-{[(2S)-1-(but-2-ynoyl)pyrrolidin-2-yl]methoxy}pyridin-4-yl)-3-[(2-ethyl-3-fluorophenyl)amino]-1H,5H,6H,7H-pyrrolo[3,2-c]pyridin-4-one (compound 284)

### 161.1. Synthesis of tert-butyl (2S)-2-{[(4-{3-[(2-ethyl-3-fluorophenyl)amino]-4-oxo-1H,5H,6H,7H-pyrrolo[3,2-c]pyridin-2-yl}pyridin-3-yl)oxy]methyl} pyrrolidine-1-carboxylate

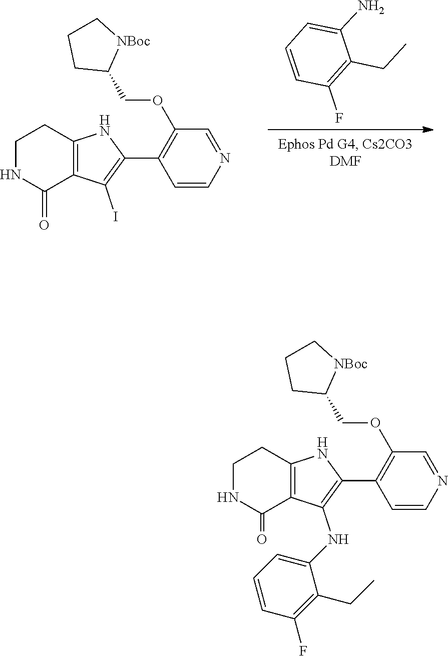

To a stirred mixture of t tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (150 mg, 0.28 mmol, 1.00 equiv) and 2-ethyl-3-fluoroaniline (116 mg, 0.84 mmol, 3.00 equiv) in DMF (1.00 mL) were added EPhos Pd G4 (26 mg, 0.03 mmol, 0.10 equiv) and Cs₂CO₃ (182 mg, 0.56 mmol, 2.00 equiv) under argon atmosphere. The resulting suspension was backfilled with argon three times and stirred for 2h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (15:1) to afford tert-butyl (2S)-2-{[(4-{3-[(2-ethyl-3-fluorophenyl)amino]-4-oxo-1H,5H,6H,7H-pyrrolo[3,2-c]pyridin-2-yl}pyridin-3-yl)oxy]methyl} pyrrolidine-1-carboxylate (150 mg, 97.95%) as a yellow solid.

LC-MS: [M+H]⁺ found 550.00.

### 161.2. Synthesis of 3-[(2-ethyl-3-fluorophenyl)amino]-2-{3-[(2S)-pyrrolidin-2-ylmethoxy]pyridin-4-yl}-1H,5H,6H,7H-pyrrolo[3,2-c]pyridin-4-one

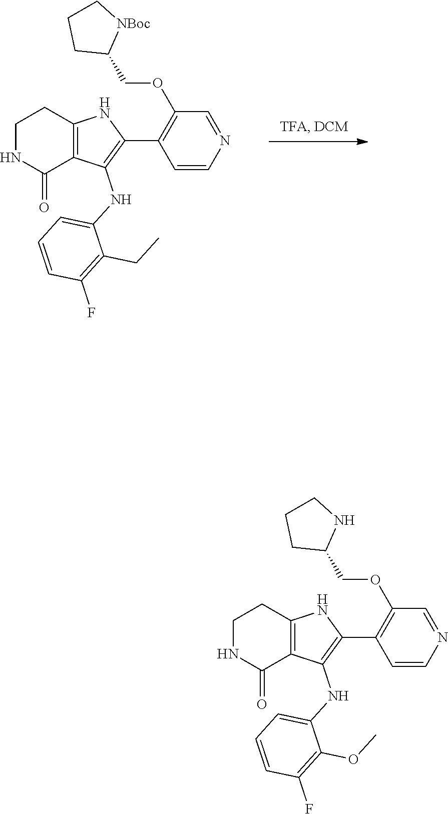 [SMILES: CCc1c(F)cccc1Nc1c(-c2ccncc2OC[C@@H]2CCCN2C(=O)OC(C)(C)C)[nH]c2c1C(=O)NCC2 ; COc1c(F)cccc1Nc1c(-c2ccncc2OC[C@@H]2CCCN2)[nH]c2c1C(=O)NCC2]

To a stirred mixture of tert-butyl (2S)-2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (100 mg, 0.18 mmol, 1.00 equiv) in DCM (1.00 mL) was added TFA (0.50 mL) at room temperature under nitrogen atmosphere. The resulting mixture was stirred for 1 h and concentrated under reduced pressure to afford 3-[(2-ethyl-3-fluorophenyl)amino]-2-{3-[(2S)-pyrrolidin-2-ylmethoxy]pyridin-4-yl}-1H,5H,6H,7H-pyrrolo[3,2-c]pyridin-4-one (100 mg, crude) as a red oil.

LC-MS: (M+H)⁺ found: 450.20.

### 161.3. Synthesis of 2-(3-{[(2S)-1-(but-2-ynoyl)pyrrolidin-2-yl]methoxy}pyridin-4-yl)-3-[(2-ethyl-3-fluorophenyl)amino]-1H,5H,6H,7H-pyrrolo[3,2-c]pyridin-4-one

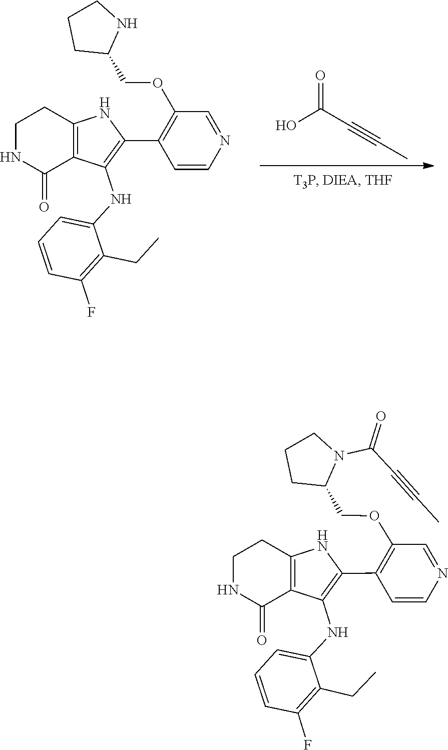

A solution of 3-[(2-ethyl-3-fluorophenyl)amino]-2-{3-[(2S)-pyrrolidin-2-ylmethoxy]pyridin-4-yl}-1H,5H,6H,7H-pyrrolo[3,2-c]pyridin-4-one (100 mg, 0.22 mmol, 1.00 equiv) in THF (3.00 mL) was basified to pH 8 with DIEA. To the above mixture was added 2-butynoic acid (28 mg, 0.33 mmol, 1.50 equiv) at 0° C. under nitrogen atmosphere followed by the addition of T₃P (142 mg, 0.44 mmol, 2.00 equiv, 50% in EA) dropwise. The resulting mixture was stirred for 1 h at room temperature under nitrogen atmosphere. Saturated aqueous NaHCO₃(10 mL) was added to the reaction mixture at 0° C. and extracted with EtOAc (3×10 mL), dried over anhydrous Na₂SO₄ and filtered. The filtrate was concentrated under reduced pressure to give crude product (60 mg) that was purified by Prep-HPLC under the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 19% B to 43% B in 8 min, 43% B; Wave Length: 254/220 nm; RT1(min): 8; Number Of Runs: 0) to afford 2-(3-{[(2S)-1-(but-2-ynoyl)pyrrolidin-2-yl]methoxy}pyridin-4-yl)-3-[(2-ethyl-3-fluorophenyl)amino]-1H,5H,6H,7H-pyrrolo[3,2-c]pyridin-4-one (51.2 mg, 44.51%) as a yellow solid.

LC-MS: (M+H)⁺ found: 516.50.

¹H NMR (400 MHz, Chloroform-d) δ 11.64 (s, 1H), 8.48 (s, 1H), 7.90 (s, 1H), 7.74 (d, 1H), 7.28 (d, 1H), 6.72-6.62 (m, 1H), 6.60 (t, 1H), 6.19 (d, 1H), 6.07 (s, 1H), 4.92 (t, 1H), 4.25 (t, 1H), 4.13 (d, 1H), 4.12-4.01 (m, 1H), 3.70-3.65 (m, 3H), 3.33-3.19 (m, 2H), 2.88-2.80 (m, 2H), 2.35-2.18 (m, 1H), 2.18-2.08 (m, 5H), 1.94-1.82 (m, 1H), 1.35 (t, 3H).

### Example 162. 3-[(3-fluoro-2-methoxyphenyl)amino]-2-(3-{[(2R)-1-(prop-2-enoyl)pyrrolidin-2-yl]methoxy}pyridin-4-yl)-1H,5H,6H,7H-pyrrolo[3,2-c]pyridin-4-one (compound 281)

### 162.1. Synthesis of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate

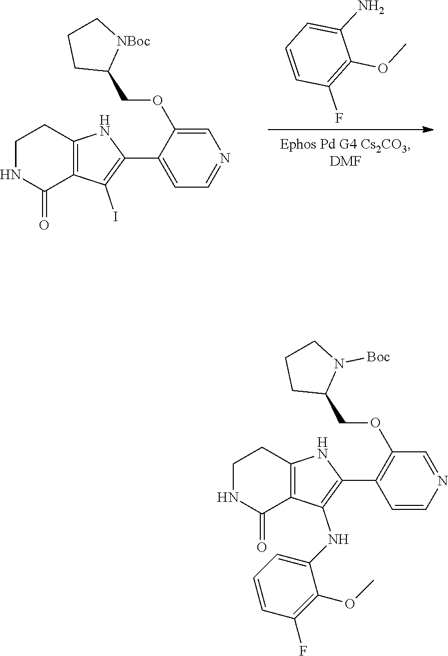

To a stirred mixture of tert-butyl (2R)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (560 mg, 1.04 mmol, 1.00 equiv) and 3-fluoro-2-methoxyaniline (293 mg, 2.08 mmol, 2.00 equiv) in DMF (10.00 mL) were added Ephos Pd G4 (95 mg, 0.10 mmol, 0.10 equiv) and Cs₂CO₃ (677 mg, 2.08 mmol, 2.00 equiv) under argon atmosphere. The resulting mixture was stirred for 2 h at 50° C. under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (10:1) to afford tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (550 mg, 95.86%) as a yellow solid.

LC-MS: (M+H)⁺ found: 552.30.

### 162.2. Synthesis of 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one

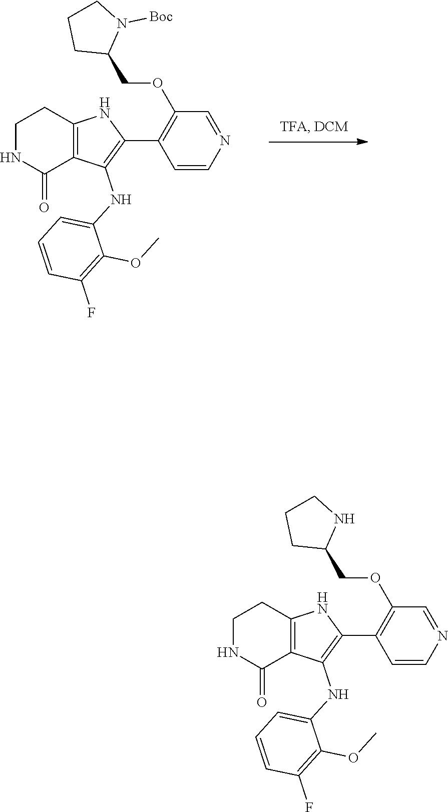 [SMILES: COc1c(F)cccc1Nc1c(-c2ccncc2OC[C@H]2CCCN2C(=O)OC(C)(C)C)[nH]c2c1C(=O)NCC2 ; COc1c(F)cccc1Nc1c(-c2ccncc2OC[C@H]2CCCN2)[nH]c2c1C(=O)NCC2]

To a stirred mixture of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (155 mg, 0.28 mmol, 1.00 equiv) in DCM (4.00 mL) was added TFA (2.00 mL) at room temperature under nitrogen atmosphere. The resulting mixture was stirred for 2 h and dried using nitrogen to afford 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one (100 mg, crude) as a red oil.

LC-MS: (M+H)⁺ found: 452.20.

### 162.3. Synthesis of 3-[(3-fluoro-2-methoxyphenyl)amino]-2-(3-{[(2R)-1-(prop-2-enoyl)pyrrolidin-2-yl]methoxy}pyridin-4-yl)-1H,5H,6H,7H-pyrrolo[3,2-c]pyridin-4-one

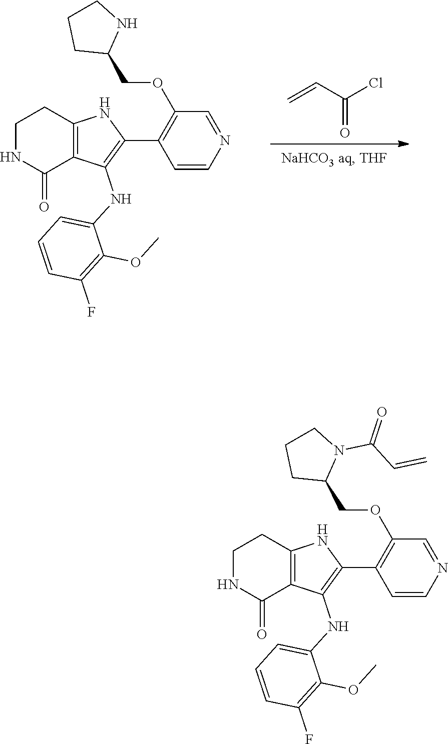

To a stirred solution of 3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one (100 mg, 0.22 mmol, 1.00 equiv) and NaHCO₃aq (2.00 mL) in THF was added acryloyl chloride (24 mg, 0.26 mmol, 1.20 equiv) dropwise at 0° C. The resulting mixture was stirred for 1.5 h at room temperature. The resulting mixture was extracted with EtOAc (3×5 mL). The combined organic layers were washed with brine (1×10 mL), dried over anhydrous Na₂SO₄. After filtration, the filtrate was concentrated under reduced pressure. The crude product (130 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 43% B in 9 min, 43% B; Wave Length: 254/220 nm; RT1(min): 8.85; Number Of Runs: 0) to afford 3-[(3-fluoro-2-methoxyphenyl)amino]-2-(3-{[(2R)-1-(prop-2-enoyl)pyrrolidin-2-yl]methoxy}pyridin-4-yl)-1H,5H,6H,7H-pyrrolo[3,2-c]pyridin-4-one (22.8 mg, 20.36%) as a yellow solid.

LC-MS: (M+H)⁺ found: 506.10.

¹H NMR (300 MHz, Chloroform-d) δ 11.62 (s, 1H), 8.20 (s, 1H), 7.93 (d, 1H), 7.76 (s, 1H), 7.53 (d, 1H), 6.61-6.47 (m, 3H), 6.42-6.36 (m, 1H), 6.04 (d, 1H), 5.81-5.77 (m, 1H), 5.21 (s, 1H), 5.08-5.05 (m, 1H), 4.26 (t, 1H), 4.13-4.09 (m, 4H), 3.76 (t, 2H), 3.64-3.60 (m, 2H), 3.23 (t, 2H), 2.17-2.11 (m, 3H), 1.89-1.86 (m, 1H).

### Example 163. 3-[(3-fluoro-2-methoxyphenyl)amino]-2-(3-{[(2S)-1-(2-fluoroprop-2-enoyl)azetidin-2-yl]methoxy}pyridin-4-yl)-1H,5H,6H,7H-pyrrolo[3,2-c]pyridin-4-one (compound 296)

### 163.1. Synthesis of 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one

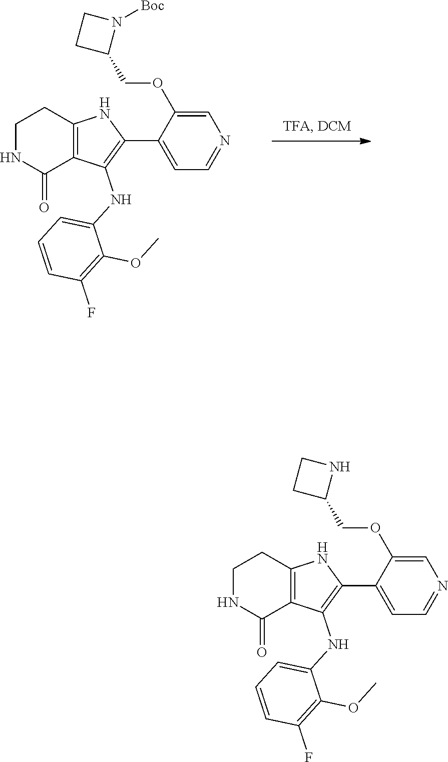 [SMILES: COc1c(F)cccc1Nc1c(-c2ccncc2OC[C@@H]2CCN2C(=O)OC(C)(C)C)[nH]c2c1C(=O)NCC2 ; COc1c(F)cccc1Nc1c(-c2ccncc2OC[C@@H]2CCN2)[nH]c2c1C(=O)NCC2]

To a stirred mixture of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (120 mg, 0.18 mmol) in DCM (4.00 mL) was added TFA (2.00 mL) at 0° C. under nitrogen atmosphere. The resulting mixture was stirred for 1 h and concentrated under reduced pressure to afford 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one (97 mg, crude) as a red oil.

LC-MS: (M+H)⁺ found: 438.05.

### 163.2. Synthesis of 3-[(3-fluoro-2-methoxyphenyl)amino]-2-(3-{[(2S)-1-(2-fluoroprop-2-enoyl)azetidin-2-yl]methoxy}pyridin-4-yl)-1H,5H,6H,7H-pyrrolo[3,2-c]pyridin-4-one

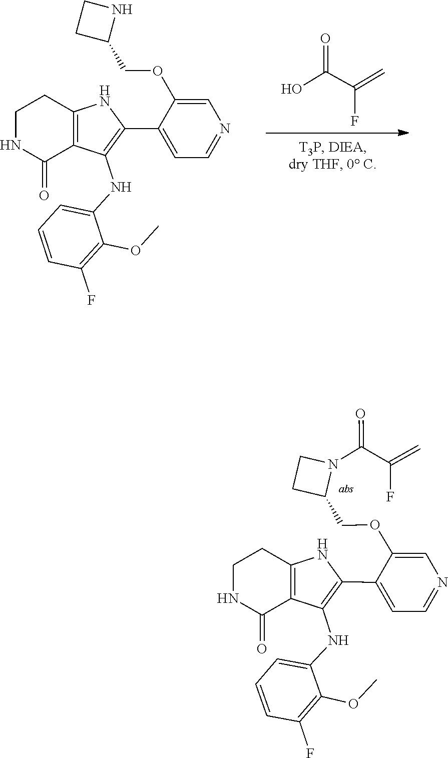

A solution of 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one (97 mg, 0.223 mmol, 1 equiv) in THF (2.00 mL) was basified to pH 8 with DIEA. To the above mixture was added 2-fluoroprop-2-enoic acid (30 mg, 0.335 mmol, 1.5 equiv) at 0° C. under nitrogen atmosphere followed by the addition of T₃P (284 mg, 0.89 mmol, 4.00 equiv, 50% in EA) dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product that was purified by Prep-HPLC under the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 32% B to 62% B in 7 min; Wave Length: 254 nm; RT1(min): 6.5; Number Of Runs: 0) to afford 3-[(3-fluoro-2-methoxyphenyl)amino]-2-(3-{[(2S)-1-(2-fluoroprop-2-enoyl)azetidin-2-yl]methoxy}pyridin-4-yl)-1H,5H,6H,7H-pyrrolo[3,2-c]pyridin-4-one (37.6 mg, 32.85%) as a yellow solid.

LC-MS: (M+H)⁺ found 510.10.

¹H NMR (300 MHz, Chloroform-d) δ 11.53 (s, 1H), 8.26 (s, 1H), 7.98 (d, 1H), 7.67 (s, 1H), 7.51 (d, 1H), 6.75-6.35 (m, 2H), 6.11-6.02 (m, 1H), 5.71-5.55 (m, 1H), 5.23-5.08 (m, 3H), 4.61-4.42 (m, 3H), 4.31-4.28 (m, 1H), 4.11 (s, 3H), 3.60 (t, 2H), 3.11 (t, 2H), 2.78-2.60 (m, 1H), 2.30-2.10 (m, 1H).

### Example 164. 2-(3-{[(2S)-1-[(2E)-4-(dimethylamino)but-2-enoyl]azetidin-2-yl]methoxy}pyridin-4-yl)-3-[(3-fluoro-2-methoxyphenyl)amino]-1H,5H,6H,7H-pyrrolo[3,2-c]pyridin-4-one 2,2,2-trifluoroacetic acid salt (compound 295)

### 164.1. Synthesis of 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one

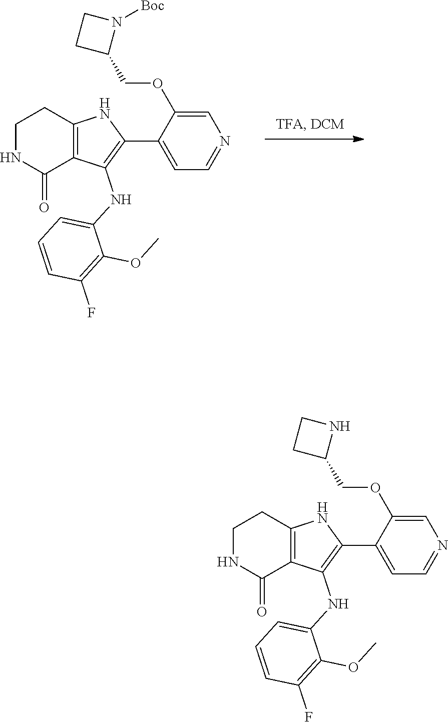 [SMILES: COc1c(F)cccc1Nc1c(-c2ccncc2OC[C@@H]2CCN2C(=O)OC(C)(C)C)[nH]c2c1C(=O)NCC2 ; COc1c(F)cccc1Nc1c(-c2ccncc2OC[C@@H]2CCN2)[nH]c2c1C(=O)NCC2]

To a stirred solution of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (80 mg, 0.15 mmol, 1.00 equiv) in DCM (2.00 mL) was added TFA (1.00 mL) dropwise at 0° C. The mixture was stirred for 1 h at room temperature under nitrogen atmosphere. The reaction was monitored by LCMS. Desired product could be detected by LCMS. The resulting mixture was concentrated under nitrogen atmosphere to afford 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one (80 mg, crude) as a red oil.

LC-MS: (M+H)⁺ found: 438.05.

### 164.2. Synthesis of 2-(3-{[(2S)-1-[(2E)-4-(dimethylamino)but-2-enoyl]azetidin-2-yl]methoxy}pyridin-4-yl)-3-[(3-fluoro-2-methoxyphenyl)amino]-1H,5H,6H,7H-pyrrolo[3,2-c]pyridin-4-one 2,2,2-trifluoroacetic acid salt

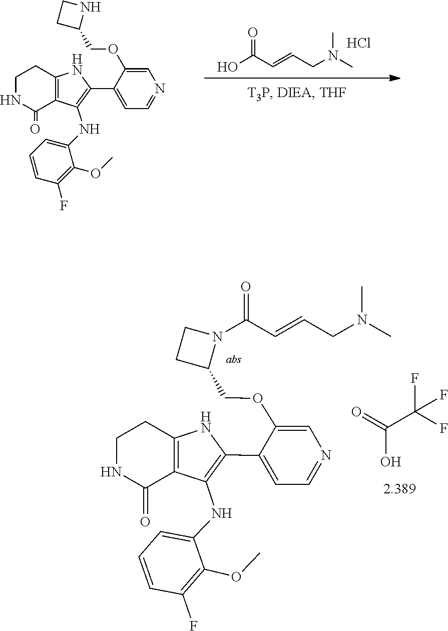

A solution of 2-{3-[(2S)-azetidin-2-ylmethoxy]pyridin-4-yl}-3-[(3-fluoro-2-methoxyphenyl)amino]-1H,5H,6H,7H-pyrrolo[3,2-c]pyridin-4-one (80 mg, 0.18 mmol, 1.00 equiv) in THF (2.00 mL).was basified to pH 8 with DIEA. To the above mixture was added (2E)-4-(dimethylamino)but-2-enoic acid (47 mg, 0.37 mmol, 2.00 equiv) at 0° C. under nitrogen atmosphere followed by the addition of T₃P (233 mg, 0.37 mmol, 2.00 equiv, 50% in EA) dropwise. The resulting mixture was stirred for overnight at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product (80 mg) that was purified by Prep-HPLC under the following conditions (Column: Xselect CSH C18 OBD Column 30*150 mm 5 m, n; Mobile Phase A: Water (0.1% TFA), Mobile Phase B: ACN; Flow rate: 60 mL/min; Gradient: 8% B to 28% B in 8 min, 28% B; Wave Length: 254/220 nm; RT1(min): 8; Number Of Runs: 0) to afford 2-(3-{[(2S)-1-[(2E)-4-(dimethylamino)but-2-enoyl]azetidin-2-yl]methoxy}pyridin-4-yl)-3-[(3-fluoro-2-methoxyphenyl)amino]-1H,5H,6H,7H-pyrrolo[3,2-c]pyridin-4-one 2,2,2-trifluoroacetic acid salt (42.1 mg, 34.71%) as a yellow solid.

LC-MS: (M+H)⁺ found: 549.5.

¹H NMR (300 MHz, DMSO-d6): δ 12.08 (s, 1H), 9.97 (s, 1H), 8.50 (s, 1H), 8.19 (d, 1H), 7.96 (s, 1H), 7.52 (d, 1H), 7.39 (s, 1H), 6.75-6.70 (m, 3H), 6.46 (d, 1H), 6.02 (d, 1H), 4.94-4.92 (m, 1H), 4.61 (t, 1H), 4.47 (d, 1H), 4.20 (t, 2H), 4.04 (s, 3H), 3.90 (d, 2H), 3.45 (t, 2H), 3.05 (t, 2H), 2.64 (s, 6H), 2.51-2.49 (m, 1H), 2.14-2.07 (m, 1H).

### Example 165. 3-[(3-fluoro-2-methoxyphenyl)amino]-2-(3-{2-[(2R)-1-(2-fluoroprop-2-enoyl)-2-methylpyrrolidin-2-yl]ethynyl}pyridin-4-yl)-1H,5H,6H,7H-pyrrolo[3,2-c]pyridin-4-one (compound 306)

### 165.1. Synthesis of 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one

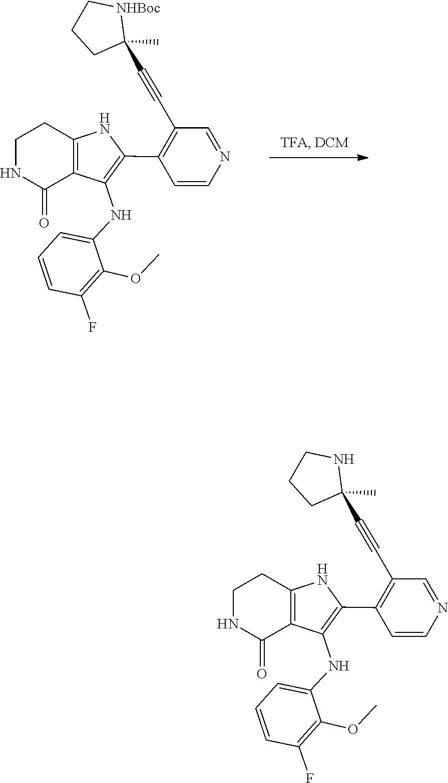

To a stirred mixture of 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (80 mg, 0.17 mmol, 1.00 equiv) in DCM (1.00 mL) was added TFA (0.30 mL) at 0° C. The resulting mixture was stirred for 1 h and dried using nitrogen to afford 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (80 mg, crude) as a red oil.

LC-MS: (M+H)⁺ found: 460.20

### 165.2. Synthesis of 3-[(3-fluoro-2-methoxyphenyl)amino]-2-(3-{2-[(2R)-1-(2-fluoroprop-2-enoyl)-2-methylpyrrolidin-2-yl]ethynyl}pyridin-4-yl)-1H,5H,6H,7H-pyrrolo[3,2-c]pyridin-4-one

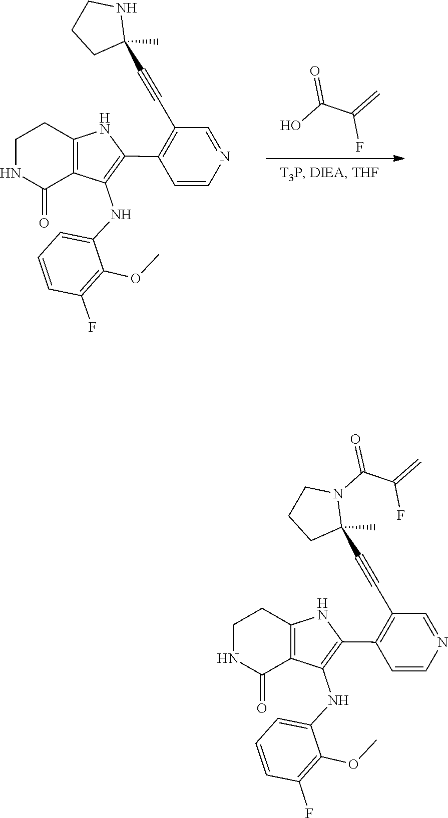

A solution of 3-[(3-fluoro-2-methoxyphenyl)amino]-2-(3-{2-[(2R)-2-methylpyrrolidin-2-yl]ethynyl}pyridin-4-yl)-1H,5H,6H,7H-pyrrolo[3,2-c]pyridin-4-one (80 mg, 0.17 mmol, 1.00 equiv) in THF (1.00 mL) was basified to pH 8 with DIEA. To the above mixture was added 2-fluoroprop-2-enoic acid (23 mg, 0.26 mmol, 1.50 equiv) at 0° C. under nitrogen atmosphere followed by the addition of T₃P (111 mg, 0.35 mmol, 2.00 equiv, 50% in EA) dropwise. The resulting mixture was stirred for 1 h at room temperature under nitrogen atmosphere. Saturated aqueous NaHCO₃(5 mL) was added to the reaction mixture at 0° C. and extracted with EtOAc (3×5 mL), dried over anhydrous Na₂SO₄ and filtered. The filtrate was concentrated under reduced pressure to give crude product. The crude product was purified by Prep-HPLC with the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 26% B to 44% B in 8 min, 44% B; Wave Length: 254/220 nm; RT1(min): 8; Number Of Runs: 0) to afford 3-[(3-fluoro-2-methoxyphenyl)amino]-2-(3-{2-[(2R)-1-(2-fluoroprop-2-enoyl)-2-methylpyrrolidin-2-yl]ethynyl}pyridin-4-yl)-1H,5H,6H,7H-pyrrolo[3,2-c]pyridin-4-one (24.5 mg, 26.45%) as a orange solid.

LC-MS: (M+H)⁺ found: 534.10

¹H NMR (300 MHz, Chloroform-d) δ 11.53 (s, 1H), 8.66 (s, 1H), 8.32 (s, 1H), 7.99 (d, 1H), 7.48 (d, 1H), 6.70-6.59 (m, 2H), 6.07 (d, 1H), 5.60-5.57 (m, 1H), 5.43-5.22 (m, 2H), 4.14 (s, 3H), 3.92-3.83 (m, 2H), 3.82-3.66 (m, 2H), 3.39-3.20 (m, 2H), 2.56-2.52 (m, 1H), 2.18-2.14 (m, 3H), 1.83 (s, 3H).

### Example 166. N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]but-2-ynamide (compound 302)

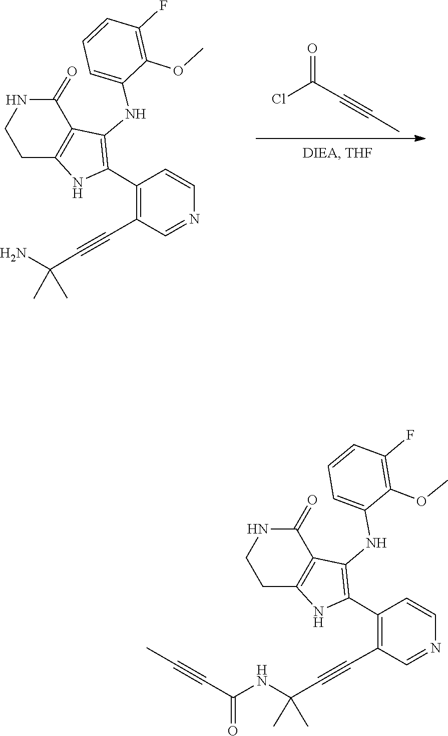

Into a 8 mL vial were added 2-[3-(3-amino-3-methylbut-1-yn-1-yl)pyridin-4-yl]-3-[(3-fluoro-2-methoxyphenyl)amino]-1H,5H,6H,7H-pyrrolo[3,2-c]pyridin-4-one (75 mg, 0.17 mmol, 1.00 equiv) and THF (2.50 mL) at room temperature. To the above mixture was added DIEA (90 mg, 0.69 mmol, 4.00 equiv) and but-2-ynoyl chloride (18 mg, 0.17 mmol, 1.00 equiv) at 0° C. The resulting mixture was stirred for additional 1 h at room temperature. The reaction was monitored by LCMS. Desired product could be detected by LCMS. The resulting mixture was extracted with CH₂Cl₂/MeOH (10:1, 4×20 mL). The combined organic layers were dried over anhydrous Na₂SO₄. After filtration, the filtrate was concentrated under reduced pressure. The crude product (102 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 28% B to 48% B in 10 min, 48% B; Wave Length: 254/220 nm; RT1(min): 9.67; Number Of Runs: 0) to afford N-[4-(4-{3-[(3-fluoro-2-methoxyphenyl)amino]-4-oxo-1H,5H,6H,7H-pyrrolo[3,2-c]pyridin-2-yl}pyridin-3-yl)-2-methylbut-3-yn-2-yl]but-2-ynamide (20.8 mg, 24.02%) as a reddish brown solid.

LC-MS: (M+H)⁺ found 500.10

¹H NMR (400 MHz, Chloroform-d) δ 11.08 (s, 1H), 8.54 (s, 1H), 8.11 (s, 1H), 7.83 (s, 1H), 7.43 (s, 1H), 6.62-6.47 (m, 2H), 6.14-6.06 (m, 2H), 5.23 (s, 1H), 4.10 (s, 3H), 3.63-3.59 (m, 2H), 3.20 (t, 2H), 1.98 (s, 3H), 1.73 (s, 6H).

### Example 167. 3-[(3-fluoro-2-methoxyphenyl)amino]-2-{3-[(1R)-1-[(2R)-1-(prop-2-enoyl)azetidin-2-yl]ethoxy]pyridin-4-yl}-1H,5H,6H,7H-pyrrolo[3,2-c]pyridin-4-one (compound 311)

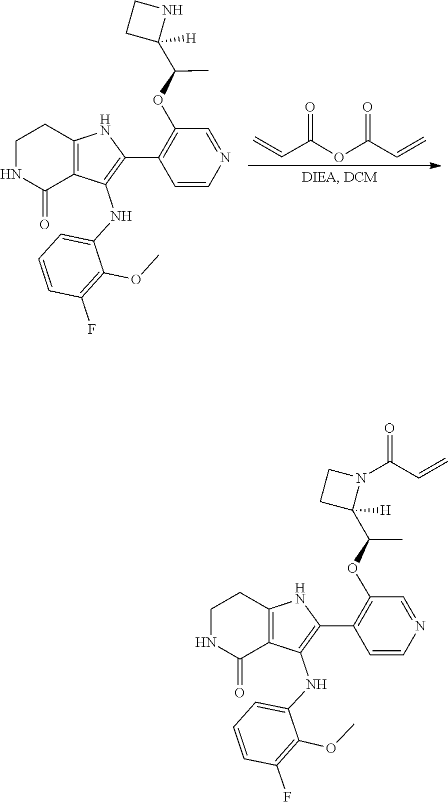

To a stirred solution of 2-{3-[(1R)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one (40 mg, 0.08 mmol, 1.00 equiv) in DCM (1.00 mL) was added DIEA (57 mg, 0.44 mmol, 5.00 equiv) and prop-2-enoyl prop-2-enoate (16 mg, 0.13 mmol, 1.50 equiv) at −40° C. under nitrogen atmosphere. The resulting mixture was stirred for 1 h at −40° C. under nitrogen atmosphere. The reaction was quenched by the addition of NaHCO₃aq (1 mL) at −40° C. The resulting mixture was extracted with EtOAc (3×5 mL). The combined organic layers were washed with brine (2×10 mL), dried over anhydrous Na₂SO₄. After filtration, the filtrate was concentrated under reduced pressure. The crude product (50 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 27% B to 43% B in 9 min, 43% B; Wave Length: 254/220 nm; RT1(min): 9; Number Of Runs: 0) to afford 3-[(3-fluoro-2-methoxyphenyl)amino]-2-{3-[(1R)-1-[(2R)-1-(prop-2-enoyl)azetidin-2-yl]ethoxy]pyridin-4-yl}-1H,5H,6H,7H-pyrrolo[3,2-c]pyridin-4-one (11 mg, 24.44%) as an off-white solid.

LC-MS: (M+H)⁺ found 506.1.

¹H NMR (400 MHz, Chloroform-d) δ 11.85 (s, 1H), 8.25 (s, 1H), 7.91 (s, 1H), 7.63 (s, 1H), 7.46 (s, 1H), 6.59-6.53 (m, 1H), 6.46 (t, 1H), 6.39-6.35 (m, 1H), 6.27-6.20 (m, 1H), 6.04 (d, 1H), 5.76 (d, 1H), 5.18 (s, 1H), 4.96-4.94 (m, 1H), 4.75-4.69 (m, 1H), 4.36-4.32 (m, 1H), 4.22-4.16 (m, 1H), 4.09 (s, 3H), 3.62-3.60 (m, 2H), 3.18-3.06 (m, 2H), 2.71-2.67 (m, 1H), 2.11 (s, 1H), 1.35 (d, 3H).

### Example 168. 2-{3-[(1R)-1-[(2R)-1-(but-2-ynoyl)azetidin-2-yl]ethoxy]pyridin-4-yl}-3-[(3-fluoro-2-methoxyphenyl)amino]-1H,5H,6H,7H-pyrrolo[3,2-c]pyridin-4-one (compound 312)

### 168.1. Synthesis of tert-butyl 2-[(1R)-1-[(4-bromopyridin-3-yl)oxy]ethyl]azetidine-1-carboxylate

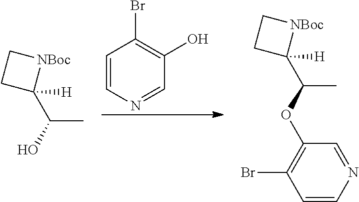

To a stirred mixture of tert-butyl (2S)-2-[(1S)-1-hydroxyethyl]azetidine-1-carboxylate (730 mg, 3.62 mmol, 1.00 equiv) and 4-bromopyridin-3-ol (631 mg, 3.62 mmol, 1.00 equiv) in THF (7.00 mL) was added PPh₃ (1.43 g, 5.44 mmol, 1.50 equiv) at 0° C. under nitrogen atmosphere. The resulting mixture was stirred for 5 min at 0° C. under nitrogen atmosphere. To the above mixture was added DEAD (947 mg, 5.44 mmol, 1.50 equiv) dropwise at 0° C. The resulting mixture was stirred for additional 3 h at 0° C. The reaction was monitored by LCMS. LCMS showed the reaction was completed. The resulting mixture was concentrated under reduced pressure. The crude product was purified by reverse phase flash with the following conditions (Mobile Phase A: Water (10 mmol/L NH₄HCO₃), Mobile Phase B: ACN; Gradient: 0% B to 100% B in 50 min) to afford tert-butyl 2-[(1R)-1-[(4-bromopyridin-3-yl)oxy]ethyl]azetidine-1-carboxylate (250 mg, 19.29%) as a colorless oil.

LC-MS: (M+H)⁺ found 359.1.

### 168.2. Synthesis of tert-butyl (2R)-2-[(1R)-1-[(4-{4-oxo-1H,5H,6H,7H-pyrrolo[3,2-c]pyridin-2-yl}pyridin-3-yl)oxy]ethyl]azetidine-1-carboxylate

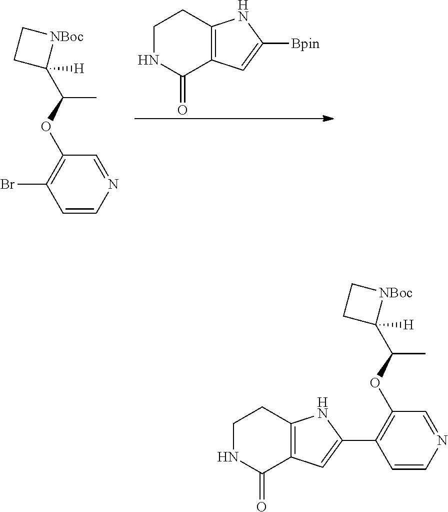

To a stirred mixture of tert-butyl 2-[(1R)-1-[(4-bromopyridin-3-yl)oxy]ethyl]azetidine-1-carboxylate (250 mg, 0.70 mmol, 1.00 equiv) and 2-(4,4,5,5-tetramethyl-1,3,2-dioxaborolan-2-yl)-1H,5H,6H,7H-pyrrolo[3,2-c]pyridin-4-one (220 mg, 0.84 mmol, 1.20 equiv) in 1,4-dioxane (3.00 mL) and H₂O (0.60 mL) was added Pd(DtBPF)Cl₂ (45 mg, 0.07 mmol, 0.10 equiv) and Na₂CO₃ (222 mg, 2.10 mmol, 3.00equiv) at room temperature under nitrogen atmosphere. The resulting mixture was stirred for additional 2 h at 50° C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford tert-butyl (2R)-2-[(1R)-1-[(4-{4-oxo-1H,5H,6H,7H-pyrrolo[3,2-c]pyridin-2-yl}pyridin-3-yl)oxy]ethyl]azetidine-1-carboxylate (270 mg, 93.54%) as a yellow solid.

LC-MS: (M+H)⁺ found 413.05.

### 168.3. Synthesis of tert-butyl (2R)-2-[(1R)-1-[(4-{3-iodo-4-oxo-1H,5H,6H,7H-pyrrolo[3,2-c]pyridin-2-yl}pyridin-3-yl)oxy]ethyl]azetidine-1-carboxylate

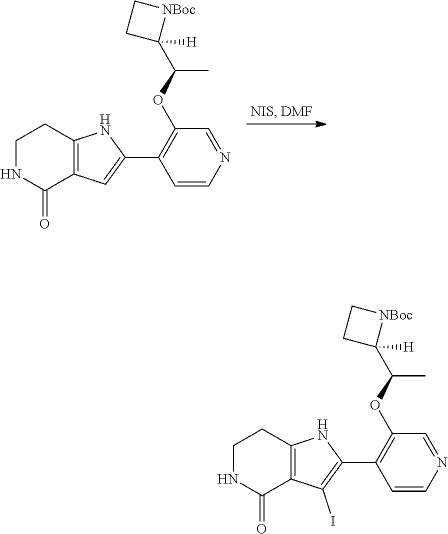 [SMILES: C[C@@H](Oc1cnccc1-c1cc2c([nH]1)CCNC2=O)[C@H]1CCN1C(=O)OC(C)(C)C ; C[C@@H](Oc1cnccc1-c1[nH]c2c(c1I)C(=O)NCC2)[C@H]1CCN1C(=O)OC(C)(C)C]

To a stirred solution of tert-butyl (2R)-2-[(1R)-1-[(4-{4-oxo-1H,5H,6H,7H-pyrrolo[3,2-c]pyridin-2-yl}pyridin-3-yl)oxy]ethyl]azetidine-1-carboxylate (270 mg, 0.65 mmol, 1.00 equiv) in DMF (5.00 mL) was added N-iodosuccinimide (161 mg, 0.72 mmol, 1.10 equiv) in portions at 0° C. under nitrogen atmosphere. The resulting mixture was stirred for 1.5 h at room temperature under nitrogen atmosphere. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford tert-butyl (2R)-2-[(1R)-1-[(4-{3-iodo-4-oxo-1H,5H,6H,7H-pyrrolo[3,2-c]pyridin-2-yl}pyridin-3-yl)oxy]ethyl]azetidine-1-carboxylate (310 mg, 87.97%) as a yellow solid.

LC-MS: (M+H)⁺ found 539.00.

### 168.4. Synthesis of tert-butyl (2R)-2-[(1R)-1-[(4-{3-[(3-fluoro-2-methoxyphenyl)amino]-4-oxo-1H,5H,6H,7H-pyrrolo[3,2-c]pyridin-2-yl}pyridin-3-yl)oxy]ethyl]azetidine-1-carboxylate

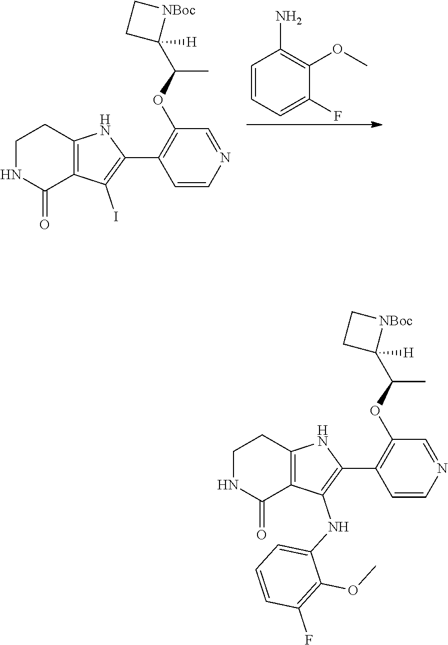

To a stirred mixture of tert-butyl (2R)-2-[(1R)-1-[(4-{3-iodo-4-oxo-1H,5H,6H,7H-pyrrolo[3,2-c]pyridin-2-yl}pyridin-3-yl)oxy]ethyl]azetidine-1-carboxylate (310 mg, 0.57 mmol, 1.00 equiv) and 3-fluoro-2-methoxyaniline (243 mg, 1.72 mmol, 3.00 equiv) in DMF (6.00 mL) was added EPhos Pd G4 (52 mg, 0.05 mmol, 0.10 equiv) and Cs₂CO₃ (375 mg, 1.15 mmol, 2.00 equiv) at room temperature under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DMF (2×0.50 mL). The resulting mixture was concentrated under reduced pressure. The residue was purified by silica gel column chromatography, eluted with CH₂Cl₂/MeOH (10:1) to afford tert-butyl (2R)-2-[(1R)-1-[(4-{3-[(3-fluoro-2-methoxyphenyl)amino]-4-oxo-1H,5H,6H,7H-pyrrolo[3,2-c]pyridin-2-yl}pyridin-3-yl)oxy]ethyl]azetidine-1-carboxylate (250 mg, 78.71%) as a brown solid.

LC-MS: (M+H)⁺ found 552.15.

### 168.5. Synthesis of 2-{3-[(1R)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one

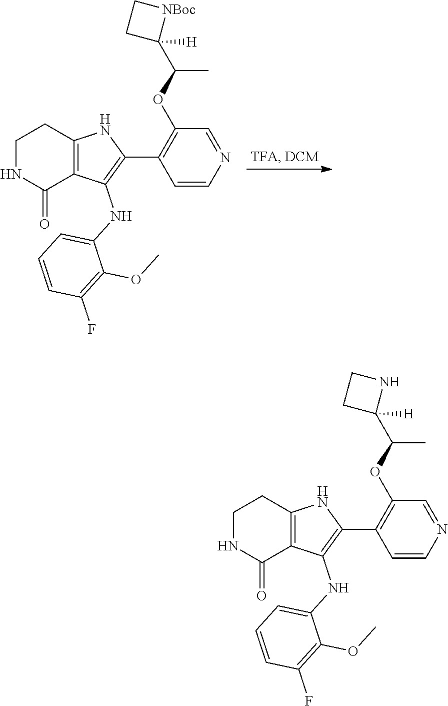 [SMILES: COc1c(F)cccc1Nc1c(-c2ccncc2O[C@H](C)[C@H]2CCN2C(=O)OC(C)(C)C)[nH]c2c1C(=O)NCC2 ; COc1c(F)cccc1Nc1c(-c2ccncc2O[C@H](C)[C@H]2CCN2)[nH]c2c1C(=O)NCC2]

To a stirred solution of tert-butyl (2R)-2-[(1R)-1-[(4-{3-[(3-fluoro-2-methoxyphenyl)amino]-4-oxo-1H,5H,6H,7H-pyrrolo[3,2-c]pyridin-2-yl}pyridin-3-yl)oxy]ethyl]azetidine-1-carboxylate (250 mg, 0.45 mmol, 1.00 equiv) in DCM (3.00 mL) was added TFA (1.00 mL) at 0° C. under nitrogen atmosphere. The resulting mixture was stirred for 1 h at room temperature under nitrogen atmosphere. The resulting mixture was concentrated under reduced pressure. The crude product was purified by reverse phase flash with the following conditions (Mobile Phase A: Water (10 mmol/L NH₄HCO₃), Mobile Phase B: ACN; Gradient: 0% B to 100% B in 50 min,) to afford 2-{3-[(1R)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one (120 mg, 58.64%) as an off-white solid.

LC-MS: (M+H)⁺ found 452.05.

### 168.6. Synthesis of 2-{3-[(1R)-1-[(2R)-1-(but-2-ynoyl)azetidin-2-yl]ethoxy]pyridin-4-yl}-3-[(3-fluoro-2-methoxyphenyl)amino]-1H,5H,6H,7H-pyrrolo[3,2-c]pyridin-4-one

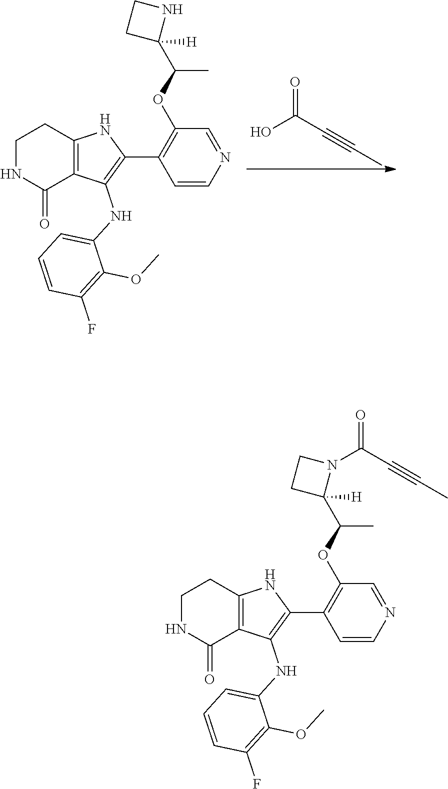

To a stirred solution of 2-{3-[(1R)-1-[(2R)-azetidin-2-yl]ethoxy]pyridin-4-yl}-3-[(3-fluoro-2-methoxyphenyl)amino]-1H,5H,6H,7H-pyrrolo[3,2-c]pyridin-4-one (80 mg, 0.17 mmol, 1.00 equiv) in THF (0.50 mL) was added DIEA (0.50 mL) at 0° C. under nitrogen atmosphere. The resulting mixture was stirred for 10 min at 0° C. under nitrogen atmosphere. To the above mixture was added 2-butynoic acid (22 mg, 0.26 mmol, 1.50 equiv) and T₃P (563 mg, 0.88 mmol, 5.00 equiv, 50% in EA) dropwise at 0° C. The resulting mixture was stirred for additional 1 h at room temperature. Saturated aqueous NaHCO₃(10 mL) was added to the reaction mixture at 0° C. and extracted with EtOAc (3 15×10 mL), dried over anhydrous Na₂SO₄. After filtration, the filtrate was concentrated under reduced pressure. The crude product (100 mg) was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 25% B to 49% B in 9 min, 49% B; Wave Length: 254/220 nm; RT1(min): 8.85; Number Of Runs: 0) to afford 2-{3-[(1R)-1-[(2R)-1-(but-2-ynoyl)azetidin-2-yl]ethoxy]pyridin-4-yl}-3-[(3-fluoro-2-methoxyphenyl)amino]-1H,5H,6H,7H-pyrrolo[3,2-c]pyridin-4-one (27 mg, 29.18%) as an off-white solid.

LC-MS: (M+H)⁺ found 518.15.

¹H NMR (400 MHz, Chloroform-d) δ 11.47 (s, 1H), 8.28 (s, 1H), 7.93 (s, 1H), 7.51-7.46 (m, 2H), 6.56-6.43 (m, 2H), 6.02 (d, 1H), 5.21 (s, 1H), 4.96-4.92 (m, 1H), 4.63-4.61 (m, 1H), 4.23 (t, 2H), 4.09 (d, 3H), 3.60-3.56 (m, 2H), 3.18-3.14 (m, 1H), 3.04-2.98 (m, 1H), 2.70-2.66 (m, 1H), 2.12-2.07 (m, 1H), 2.02 (s, 3H), 1.32 (d, 3H).

### Example 169. rel-N-[(3R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide (compound 323)

### 169.1. Synthesis of tert-butyl N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl}carbamate

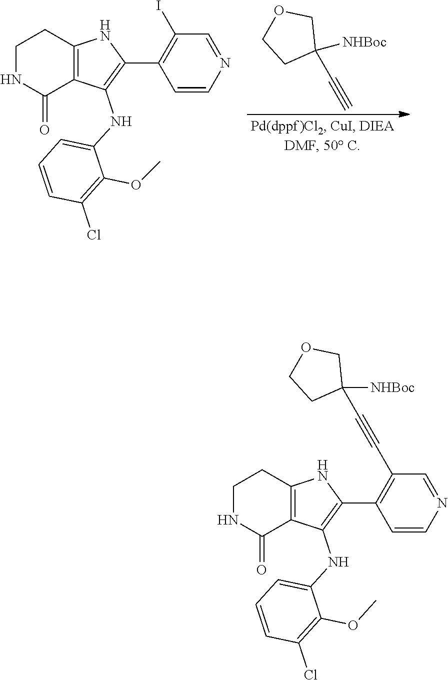

To a stirred solution of 3-[(3-chloro-2-methoxyphenyl)amino]-2-(3-iodopyridin-4-yl)-1H,5H,6H,7H-pyrrolo[3,2-c]pyridin-4-one (350 mg, 0.70 mmol, 1.00 equiv) and Pd(dppf)Cl₂·CH₂Cl₂ (144 mg, 0.17 mmol, 0.25 equiv) and CuI (67 mg, 0.35 mmol, 0.50 equiv) in DMF (3.00 mL) were added tert-butyl N-(3-ethynyloxolan-3-yl)carbamate (373 mg, 1.76 mmol, 2.50 equiv) and DIEA (457 mg, 3.53 mmol, 5.00 equiv) at room temperature under Ar atmosphere. The resulting mixture was stirred for 2 h at 50° C. under Ar atmosphere in a sealed tube. The residue was purified by reverse flash chromatography with the following conditions (column, C18 gel; mobile phase, MeCN in water, 10% to 80% gradient in 30 min; detector, UV 254 nm) to afford tert-butyl N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl}carbamate (270 mg, 66.02%) as a yellow solid.

LC-MS: (M+H)⁺ found: 578.15.

### 169.2. Synthesis of 2-{3-[2-(3-aminooxolan-3-yl)ethynyl]pyridin-4-yl}-3-[(3-chloro-2-methoxyphenyl)amino]-1H,5H,6H,7H-pyrrolo[3,2-c]pyridin-4-one

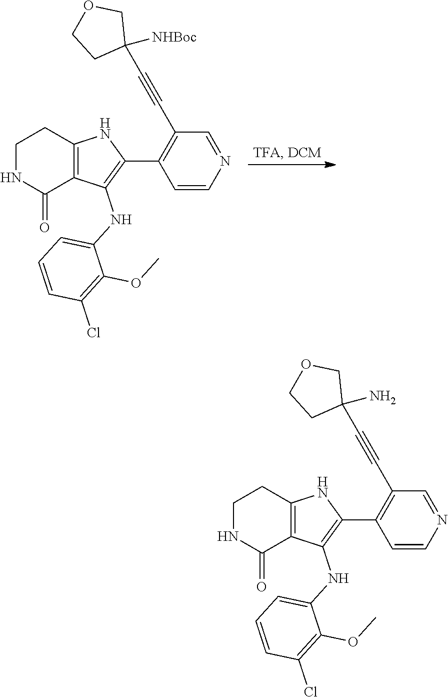 [SMILES: COc1c(Cl)cccc1Nc1c(-c2ccncc2C#CC2(NC(=O)OC(C)(C)C)CCOC2)[nH]c2c1C(=O)NCC2 ; COc1c(Cl)cccc1Nc1c(-c2ccncc2C#CC2(N)CCOC2)[nH]c2c1C(=O)NCC2]

To a stirred solution of tert-butyl N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl}carbamate (240 mg, 0.41 mmol, 1 equiv) in DCM (2.00 mL) was added TFA (2.00 mL) dropwise at 0° C. The resulting mixture was stirred for 2 h at room temperature. The resulting mixture was worked up and dried using nitrogen. The residue was purified by reverse flash chromatography with the following conditions (column, C18 gel; mobile phase, MeOH in water, 10% to 60% gradient in 10 min; detector, UV 254 nm) to afford 2-{3-[2-(3-aminooxolan-3-yl)ethynyl]pyridin-4-yl}-3-[(3-chloro-2-methoxyphenyl)amino]-1H,5H,6H,7H-pyrrolo[3,2-c]pyridin-4-one (130 mg, 65.51%) as a yellow solid.

LC-MS: (M+H)⁺ found: 478.05.

### 169.3. Synthesis of N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl}prop-2-enamide

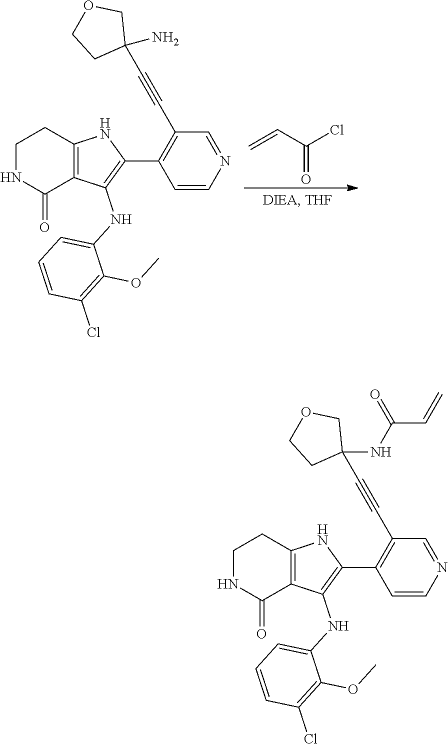

To a stirred solution of 2-{3-[2-(3-aminooxolan-3-yl)ethynyl]pyridin-4-yl}-3-[(3-chloro-2-methoxyphenyl)amino]-1H,5H,6H,7H-pyrrolo[3,2-c]pyridin-4-one (125 mg, 0.26 mmol, 1.00 equiv) and DIEA (169 mg, 1.31 mmol, 5.00 equiv) in THF (2.50 mL) was added acryloyl chloride (35 mg, 0.39 mmol, 1.50 equiv)/THF (0.50 mL) dropwise at 0° C. The aqueous layer was extracted with EtOAc (3×5 mL). The combined organic layers were washed with brine, dried over anhydrous Na₂SO₄. After filtration, the filtrate was concentrated under reduced pressure. The residue was then purified by Prep-HPLC with the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 16% B to 36% B in 10 min, 36% B; Wave Length: 254/220 nm; RT1(min): 9; Number Of Runs: 0) to afford N-{3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl}prop-2-enamide (40 mg, 28.75%) as a orange solid.

LC-MS: (M+H)⁺ found: 532.1.

### 169.4. Synthesis of rel-N-[(3R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide

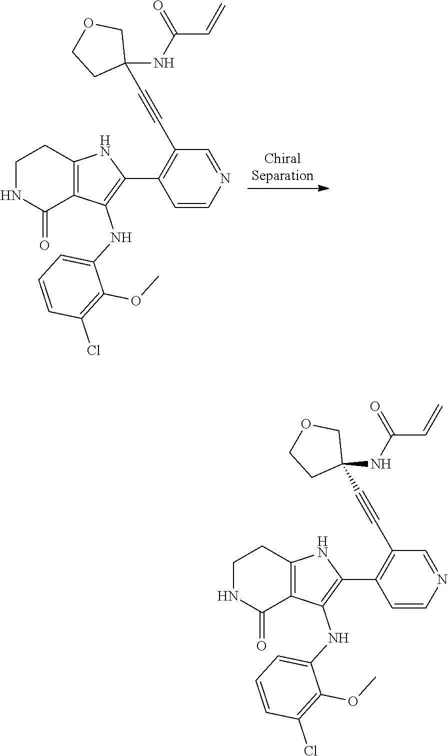 [SMILES: C=CC(=O)NC1(C#Cc2cnccc2-c2[nH]c3c(c2Nc2cccc(Cl)c2OC)C(=O)NCC3)CCOC1 ; C=CC(=O)N[C@@]1(C#Cc2cnccc2-c2[nH]c3c(c2Nc2cccc(Cl)c2OC)C(=O)NCC3)CCOC1]

The racemic product (40 mg) was purified by Prep-HPLC with the following conditions (Column: CHIRALPAK IF, 2*25 cm, 5 μm; Flow rate: 20 mL/min; Gradient: 15% B to 15% B in 27 min; Wave Length: 220/254 nm; RT1(min): 23.53; RT2(min): 29.97; Injection Volume: 0.875 mL; Number Of Runs: 4) to afford rel-N-[(3R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide (6.8 mg, 16.91%) as a orange solid.

LC-MS: (M+H)⁺ found: 532.05.

¹H NMR (400 MHz, Chloroform-d) δ 11.37 (s, 1H), 8.59 (s, 1H), 8.10-7.99 (m, 2H), 7.40 (d, 1H), 6.82-6.79 (m, 1H), 6.67-6.60 (m, 2H), 6.37-6.32 (m, 1H), 6.25-6.18 (m, 2H), 5.78-5.76 (m, 1H), 5.38 (s, 1H), 4.20 (d, 1H), 4.21-4.07 (m, 6H), 3.68-3.64 (m, 2H), 3.28 (t, 2H), 2.64-2.57 (m, 2H).

### Example 170. rel-N-[(3R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide (compound 322)

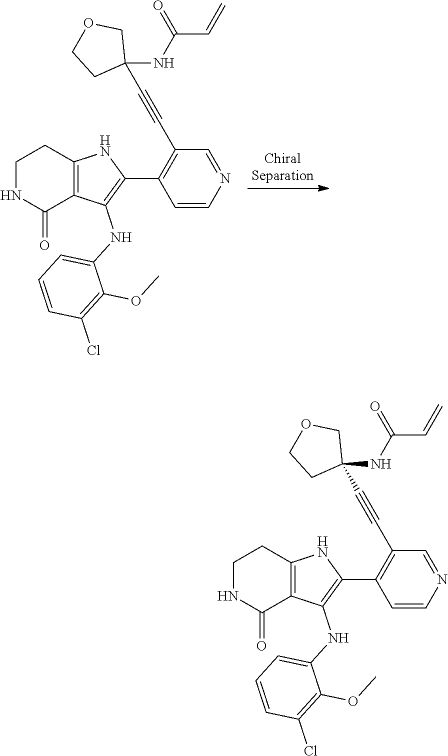 [SMILES: C=CC(=O)NC1(C#Cc2cnccc2-c2[nH]c3c(c2Nc2cccc(Cl)c2OC)C(=O)NCC3)CCOC1 ; C=CC(=O)N[C@@]1(C#Cc2cnccc2-c2[nH]c3c(c2Nc2cccc(Cl)c2OC)C(=O)NCC3)CCOC1]

The racemic product (40 mg) was purified by Prep-HPLC with the following conditions (Column: CHIRALPAK IF, 2*25 cm, 5 μm; Flow rate: 20 mL/min; Gradient: 15% B to 15% B in 27 min; Wave Length: 220/254 nm; RT1(min): 23.53; RT2(min): 29.97; Injection Volume: 0.875 mL; Number Of Runs: 4) to afford rel-N-[(3R)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide (9.3 mg, 23.13%) as a orange solid.

LC-MS: (M+H)⁺ found: 532.05.

¹H NMR (400 MHz, Chloroform-d) δ 11.46 (s, 1H), 8.50 (s, 1H), 8.29 (s, 1H), 7.96 (d, 1H), 7.37 (d, 1H), 6.85-6.83 (m, 2H), 6.67 (t, 1H), 6.33-6.19 (m, 3H), 5.77 (d, 1H), 5.33 (s, 1H), 4.20 (d, 1H), 4.11-4.08 (m, 6H), 3.68-3.65 (m, 2H), 3.31 (t, 2H), 2.62 (t, 2H).

### Example 171. rel-N-[(3R)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide (compound 321)

### 171.1. Synthesis of tert-butyl N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl}carbamate

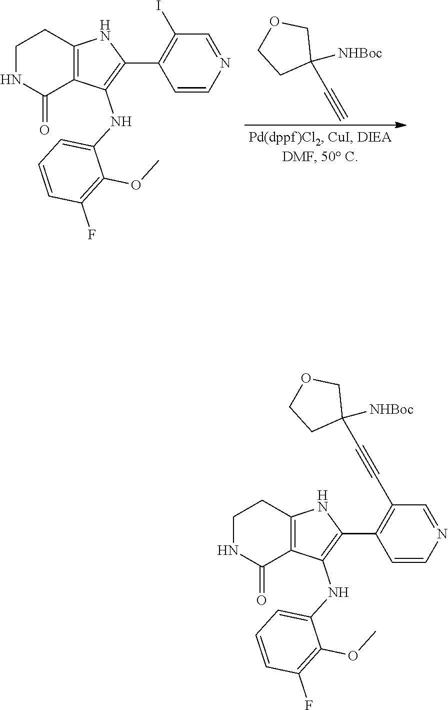

To a stirred solution of 3-[(3-fluoro-2-methoxyphenyl)amino]-2-(3-iodopyridin-4-yl)-1H,5H,6H,7H-pyrrolo[3,2-c]pyridin-4-one (330 mg, 0.69 mmol, 1.00 equiv) and Pd(dppf)C12-CH₂Cl₂ (140 mg, 0.17 mmol, 0.25 equiv) and CuI (65 mg, 0.34 mmol, 0.50 equiv) in DMF (3.00 mL) were added tert-butyl N-(3-ethynyloxolan-3-yl)carbamate (349 mg, 1.65 mmol, 2.40 equiv) and DIEA (445 mg, 3.45 mmol, 5.00 equiv) at room temperature under Ar atmosphere. The resulting mixture was stirred for 2 h at 50° C. under Ar atmosphere in a sealed tube. The residue was purified by reverse flash chromatography with the following conditions (column, C18 gel; mobile phase, MeCN in water, 10% to 80% gradient in 30 min; detector, UV 254 nm) to afford tert-butyl N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl}carbamate (320 mg, 82.58%) as a yellow solid.

LC-MS: (M+H)⁺ found: 562.20.

### 171.2. Synthesis of 2-{3-[2-(3-aminooxolan-3-yl)ethynyl]pyridin-4-yl}-3-[(3-fluoro-2-methoxyphenyl)amino]-1H,5H,6H,7H-pyrrolo[3,2-c]pyridin-4-one

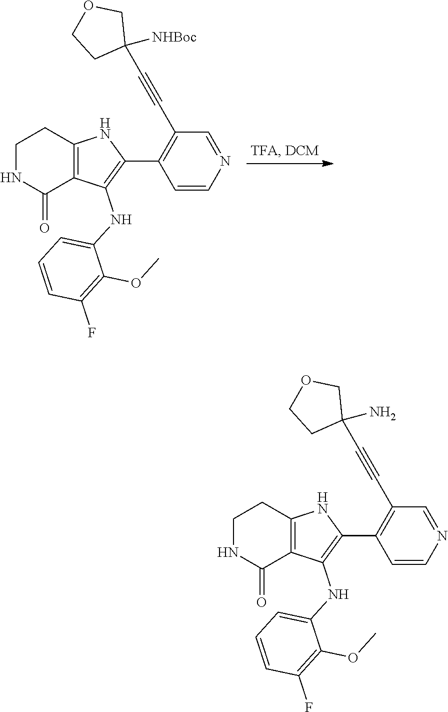 [SMILES: COc1c(F)cccc1Nc1c(-c2ccncc2C#CC2(NC(=O)OC(C)(C)C)CCOC2)[nH]c2c1C(=O)NCC2 ; COc1c(F)cccc1Nc1c(-c2ccncc2C#CC2(N)CCOC2)[nH]c2c1C(=O)NCC2]

To a stirred solution of tert-butyl N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl}carbamate (310 mg, 0.55 mmol, 1.00 equiv) in DCM (3.00 mL) was added TFA (3.00 mL) dropwise at 0° C. The resulting mixture was stirred for 2 h at room temperature. The resulting mixture was worked up and dried using nitrogen. The residue was purified by reverse flash chromatography with the following conditions (column, C18 gel; mobile phase, MeOH in water, 10% to 60% gradient in 10 min; detector, UV 254 nm) to afford 2-{3-[2-(3-aminooxolan-3-yl)ethynyl]pyridin-4-yl}-3-[(3-fluoro-2-methoxyphenyl)amino]-1H,5H,6H,7H-pyrrolo[3,2-c]pyridin-4-one (190 mg, 74.59%) as a yellow solid.

LC-MS: (M+H)⁺ found: 462.10.

### 171.3. Synthesis of N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl}prop-2-enamide

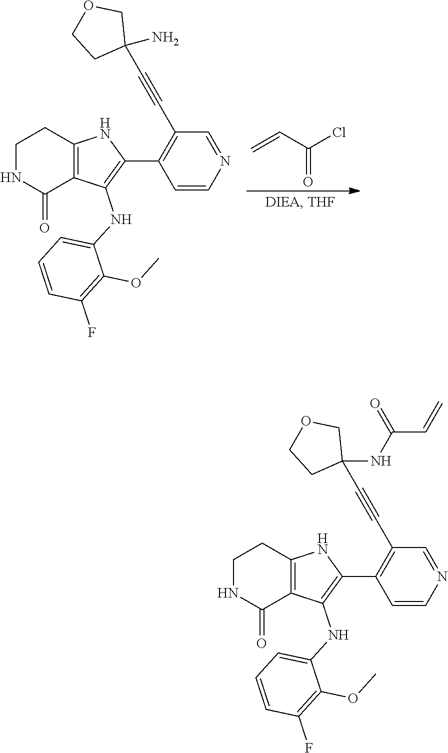

To a stirred solution of 2-{3-[2-(3-aminooxolan-3-yl)ethynyl]pyridin-4-yl}-3-[(3-fluoro-2-methoxyphenyl)amino]-1H,5H,6H,7H-pyrrolo[3,2-c]pyridin-4-one (170 mg, 0.36 mmol, 1.00 equiv) and DIEA (238 mg, 1.84 mmol, 5.00 equiv) in THF (2.50 mL) was added acryloyl chloride (66 mg, 0.73 mmol, 2.00 equiv)/THF (0.50 mL) dropwise at 0° C. The aqueous layer was extracted with EtOAc (3×5 mL). The combined organic layers were washed with brine, dried over anhydrous Na₂SO₄. After filtration, the filtrate was concentrated under reduced pressure. The residue was purified by silica gel column chromatography, eluted with CH₂Cl₂/MeOH (10:1) to afford N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl}prop-2-enamide (120 mg) as a yellow solid. The product (120 mg) was then purified by Prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 12% B to 32% B in 13 min, 32% B; Wave Length: 254 nm; RT1(min): 12.4; Number Of Runs: 0) to afford N-{3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl}prop-2-enamide (80 mg, 42.13%) as a orange solid.

LC-MS: (M+H)⁺ found: 516.2.

### 171.4. Synthesis of rel-N-[(3R)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide

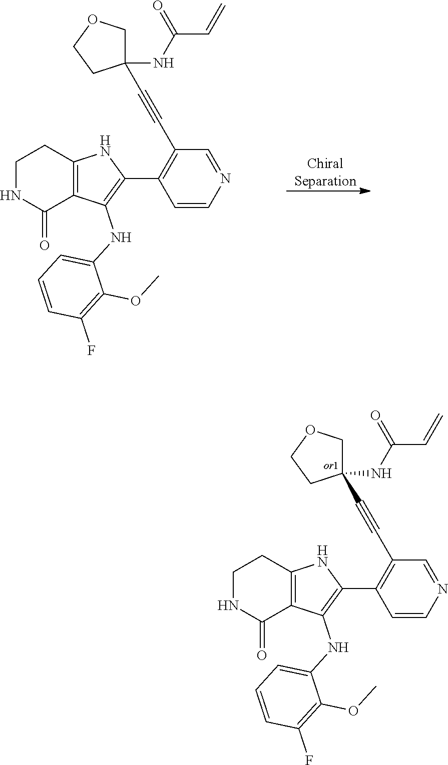 [SMILES: C=CC(=O)NC1(C#Cc2cnccc2-c2[nH]c3c(c2Nc2cccc(F)c2OC)C(=O)NCC3)CCOC1 ; C=CC(=O)N[C@]1(C#Cc2cnccc2-c2[nH]c3c(c2Nc2cccc(F)c2OC)C(=O)NCC3)CCOC1]

The racemic product (80 mg) was purified by Chiral Prep-HPLC with the following conditions (Column: CHIRALPAK IF, 2*25 cm, 5 μm; Flow rate: 20 mL/min; Gradient: 15% B to 15% B in 27 min; Wave Length: 220/254 nm; RT1(min): 23.53; RT2(min): 29.97; Injection Volume: 0.875 mL; Number Of Runs: 4) to afford rel-N-[(3R)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide (20.8 mg, 26.00%) as a yellow solid.

LC-MS: (M+H)⁺ found: 516.10.

¹H NMR (300 MHz, Chloroform-d) δ 11.22 (s, 1H), 8.57 (s, 1H), 8.13 (d, 1H), 7.81 (s, 1H), 7.46 (d, 1H), 6.60-6.56 (m, 1H), 6.53-6.46 (m, 1H), 6.42-6.32 (m, 2H), 6.22-6.08 (m, 2H), 5.78-5.74 (m, 1H), 5.31 (s, 1H), 4.20 (d, 1H), 4.10-4.04 (m, 6H), 3.65-3.61 (m, 2H), 3.24 (t, 2H), 2.63-2.55 (m, 2H).

### Example 172. rel-N-[(3R)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide (compound 320)

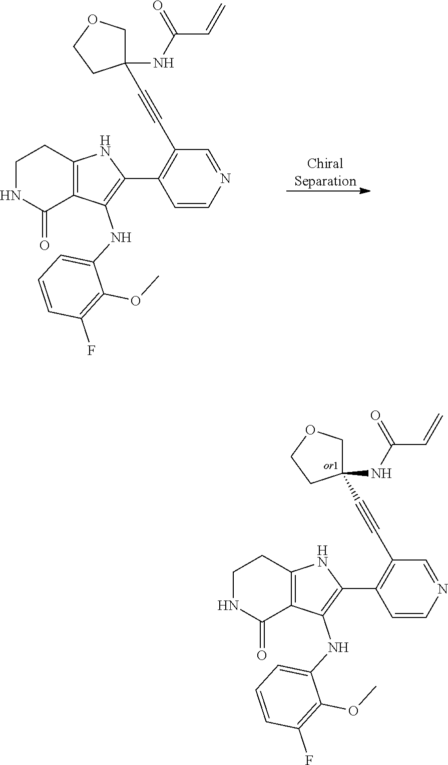 [SMILES: C=CC(=O)NC1(C#Cc2cnccc2-c2[nH]c3c(c2Nc2cccc(F)c2OC)C(=O)NCC3)CCOC1 ; C=CC(=O)N[C@@]1(C#Cc2cnccc2-c2[nH]c3c(c2Nc2cccc(F)c2OC)C(=O)NCC3)CCOC1]

The racemic product (80 mg) was purified by Prep-HPLC with the following conditions (Column: CHIRALPAK IF, 2*25 cm, 5 μm; Flow rate: 20 mL/min; Gradient: 15% B to 15% B in 27 min; Wave Length: 220/254 nm; RT1(min): 23.53; RT2(min): 29.97; Injection Volume: 0.875 mL; Number Of Runs: 4) to afford rel-N-[(3R)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]oxolan-3-yl]prop-2-enamide (23.3 mg, 29.13%) as a yellow solid.

LC-MS: (M+H)⁺ found: 516.10.

¹H NMR (300 MHz, Chloroform-d) δ 11.39 (s, 1H), 8.56 (s, 1H), 8.15 (s, 1H), 8.02 (d, 1H), 7.44 (d, 1H), 6.70-6.53 (m, 3H), 6.37-6.19 (m, 2H), 6.07 (d, 1H), 5.77 (d, 1H), 5.35 (s, 1H), 4.20 (d, 1H), 4.12-4.06 (m, 6H), 3.66 (t, 2H), 3.29 (t, 2H), 2.61 (t, 2H).

### Example 173. 3-[(3-chloro-2-methoxyphenyl)amino]-2-(3-{2-[2-(prop-2-enoyl)-2-azabicyclo[2.1.1]hexan-1-yl]ethynyl}pyridin-4-yl)-1H,5H,6H,7H-pyrrolo[3,2-c]pyridin-4-one (compound 319)

### 173.1. Synthesis of tert-butyl 1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[2.1.1]hexane-2-carboxylate

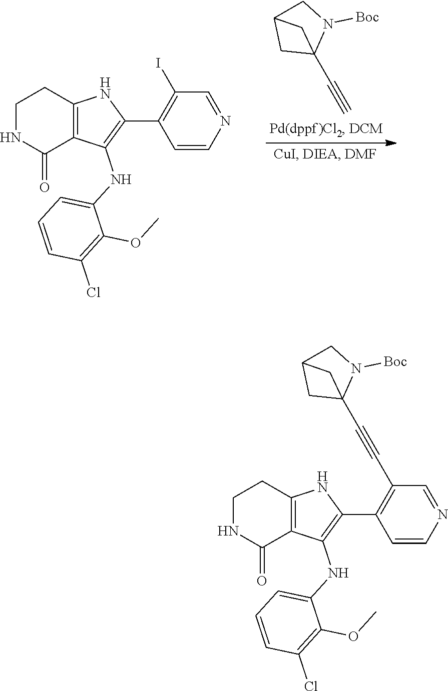 [SMILES: COc1c(Cl)cccc1Nc1c(-c2ccncc2I)[nH]c2c1C(=O)NCC2 ; C#CC12CC(CN1C(=O)OC(C)(C)C)C2 ; COc1c(Cl)cccc1Nc1c(-c2ccncc2C#CC23CC(CN2C(=O)OC(C)(C)C)C3)[nH]c2c1C(=O)NCC2]

To a stirred mixture of 3-[(3-chloro-2-methoxyphenyl)amino]-2-(3-iodopyridin-4-yl)-1H,5H,6H,7H-pyrrolo[3,2-c]pyridin-4-one (240 mg, 0.49 mmol, 1.00 equiv) and tert-butyl 1-ethynyl-2-azabicyclo[2.1.1]hexane-2-carboxylate (201 mg, 0.97 mmol, 2.00 equiv) in DMF (4.00 mL) were added Pd(dppf)C12-CH₂Cl₂ (99 mg, 0.12 mmol, 0.25 equiv), DIEA (188 mg, 1.46 mmol, 3.00 equiv) and CuI (46 mg, 0.24 mmol, 0.50 equiv) at room temperature under argon atmosphere. The resulting suspension was backfilled with argon three times and stirred for 2 h at 50° C. LCMS confirmed completion of reaction and desired product was observed. The resulting mixture was purified by reverse flash chromatography with the following conditions: column, C18 gel; mobile phase, MeCN in water, 10% to 60% gradient in 10 min; detector, UV 254 nm. The resulting mixture was concentrated under reduced pressure. The residue was purified by silica gel column chromatography, eluted with CH₂Cl₂/MeOH (20:1) to afford tert-butyl 1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[2.1.1]hexane-2-carboxylate (190 mg, 68.22%) as a yellow solid.

LC-MS: (M+H)⁺ found: 574.15.

### 173.2. Synthesis of 2-[3-(2-{2-azabicyclo[2.1.1]hexan-1-yl}ethynyl)pyridin-4-yl]-3-[(3-chloro-2-methoxyphenyl)amino]-1H,5H,6H,7H-pyrrolo[3,2-c]pyridin-4-one

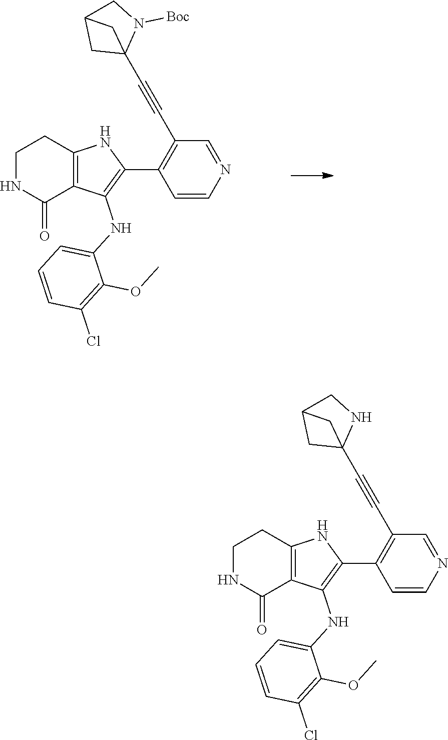 [SMILES: COc1c(Cl)cccc1Nc1c(-c2ccncc2C#CC23CC(CN2C(=O)OC(C)(C)C)C3)[nH]c2c1C(=O)NCC2 ; COc1c(Cl)cccc1Nc1c(-c2ccncc2C#CC23CC(CN2)C3)[nH]c2c1C(=O)NCC2]

To a stirred solution of tert-butyl 1-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[2.1.1]hexane-2-carboxylate (170 mg, 0.29 mmol, 1.00 equiv) and 2,6-lutidine (793 mg, 7.40 mmol, 25.00 equiv) in DCM (5.00 mL) was added TMSOTf (2.90 mL, 1.48 mmol, 5.00 equiv) dropwise at 0° C. under nitrogen atmosphere. The resulting mixture was stirred for 3 h at room temperature under nitrogen atmosphere. The resulting mixture was concentrated under reduced pressure. The residue was purified by reverse flash chromatography with the following conditions: column, C18 gel; mobile phase, MeCN in water, 10% to 50% gradient in 10 min; detector, UV 254 nm. This resulted in 2-[3-(2-{2-azabicyclo[2.1.1]hexan-1-yl}ethynyl)pyridin-4-yl]-3-[(3-chloro-2-methoxyphenyl)amino]-1H,5H,6H,7H-pyrrolo[3,2-c]pyridin-4-one (130 mg, 92.62%) as a yellow solid.

LC-MS: (M+H)⁺ found: 474.00

### 173.3. Synthesis of 3-[(3-chloro-2-methoxyphenyl)amino]-2-(3-{2-[2-(prop-2-enoyl)-2-azabicyclo[2.1.1]hexan-1-yl]ethynyl}pyridin-4-yl)-1H,5H,6H,7H-pyrrolo[3,2-c]pyridin-4-one

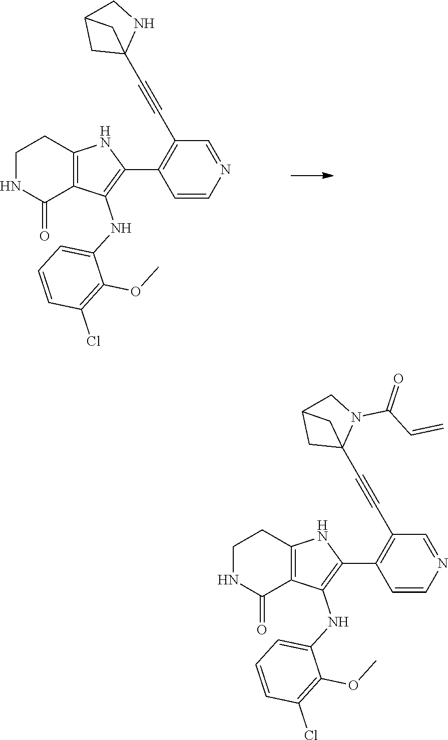 [SMILES: COc1c(Cl)cccc1Nc1c(-c2ccncc2C#CC23CC(CN2)C3)[nH]c2c1C(=O)NCC2 ; C=CC(=O)N1CC2CC1(C#Cc1cnccc1-c1[nH]c3c(c1Nc1cccc(Cl)c1OC)C(=O)NCC3)C2]

To a stirred mixture of 2-[3-(2-{2-azabicyclo[2.1.1]hexan-1-yl}ethynyl)pyridin-4-yl]-3-[(3-chloro-2-methoxyphenyl)amino]-1H,5H,6H,7H-pyrrolo[3,2-c]pyridin-4-one (110 mg, 0.23 mmol, 1.00 equiv) and DIEA (150 mg, 1.16 mmol, 5.00 equiv) in DCM (5.00 mL) 5 was added prop-2-enoyl prop-2-enoate (59 mg, 0.46 mmol, 2.00 equiv) dropwise at 0° C. under nitrogen atmosphere. The resulting mixture was stirred for 1 h at room temperature under nitrogen atmosphere. The residue was purified by silica gel column chromatography, eluted with CH₂Cl₂/MeOH (20:1) to afford 3-[(3-chloro-2-methoxyphenyl)amino]-2-(3-{2-[2-(prop-2-enoyl)-2-azabicyclo[2.1.1]hexan-1-yl]ethynyl}pyridin-4-yl)-1H,5H,6H,7H-pyrrolo[3,2-c]pyridin-4-one (30 mg, crude) as a yellow solid. The residue was purified by trituration with acetonitrile (5 mL) to afford 3-[(3-chloro-2-methoxyphenyl)amino]-2-(3-{2-[2-(prop-2-enoyl)-2-azabicyclo[2.1.1]hexan-1-yl]ethynyl}pyridin-4-yl)-1H,5H,6H,7H-pyrrolo[3,2-c]pyridin-4-one (25.1 mg, 19.99%) as a yellow solid.

LC-MS: (M+H)⁺ found: 528.05

¹H NMR (300 MHz, Chloroform-d) δ 11.53 (s, 1H), 8.61 (s, 1H), 8.14 (d, 1H), 7.74 (s, 1H), 7.42 (d, 1H), 6.76-6.67 (m, 1H), 6.65-6.53 (m, 1H), 6.53-6.37 (m, 2H), 6.29-6.18 (m, 1H), 5.86-5.75 (m, 1H), 5.24 (s, 1H), 4.07 (s, 3H), 3.68 (s, 2H), 3.62-3.51 (m, 2H), 3.07 (t, 2H), 2.96 (t, 1H), 2.46-2.32 (m, 2H), 2.00-1.92 (m, 2H).

### Example 174. 3-[(3-fluoro-2-methoxyphenyl)amino]-2-(3-{2-[2-(prop-2-enoyl)-2-azabicyclo[2.1.1]hexan-1-yl]ethynyl}pyridin-4-yl)-1H,5H,6H,7H-pyrrolo[3,2-c]pyridin-4-one (compound 318)

### 174.1. Synthesis of tert-butyl 1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[2.1.1]hexane-2-carboxylate

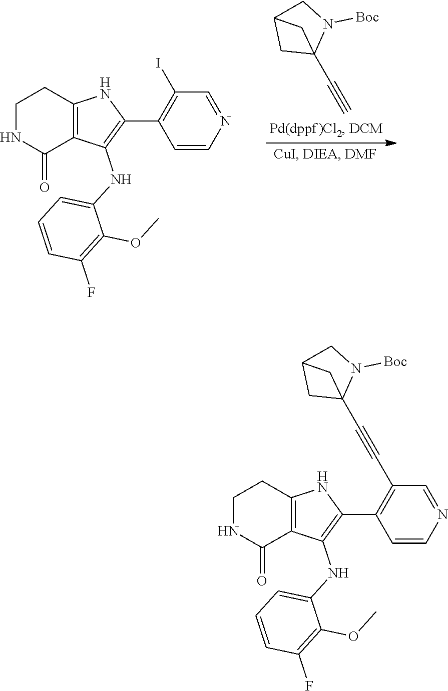 [SMILES: COc1c(F)cccc1Nc1c(-c2ccncc2I)[nH]c2c1C(=O)NCC2 ; C#CC12CC(CN1C(=O)OC(C)(C)C)C2 ; COc1c(F)cccc1Nc1c(-c2ccncc2C#CC23CC(CN2C(=O)OC(C)(C)C)C3)[nH]c2c1C(=O)NCC2]

To a stirred mixture of 3-[(3-fluoro-2-methoxyphenyl)amino]-2-(3-iodopyridin-4-yl)-1H,5H,6H,7H-pyrrolo[3,2-c]pyridin-4-one (200 mg, 0.42 mmol, 1.00 equiv) and CuI (39 mg, 0.21 mmol, 0.50 equiv) Pd(dppf)Cl₂·CH₂Cl₂ (85 mg, 0.10 mmol, 0.25 equiv) in DMF (2.00 mL) were added tert-butyl 1-ethynyl-2-azabicyclo[2.1.1]hexane-2-carboxylate (173 mg, 0.84 mmol, 2.00 equiv) and DIEA (162 mg, 1.25 mmol, 3.00 equiv) at room temperature under nitrogen atmosphere. The resulting suspension was backfilled with argon three times and stirred for 2 h at 50° C. LCMS confirmed completion of reaction and desired product was observed. The resulting mixture was purified by reverse flash chromatography with the following conditions (column, C18 gel; mobile phase, MeCN in water, 10% to 50% gradient in 10 min; detector, UV 254 nm) to afford tert-butyl 1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[2.1.1]hexane-2-carboxylate (150 mg, 64.33%) as a yellow solid.

LC-MS: (M+H)⁺ found: 558.15.

### 174.2. Synthesis of 2-[3-(2-{2-azabicyclo[2.1.1]hexan-1-yl}ethynyl)pyridin-4-yl]-3-[(3-fluoro-2-methoxyphenyl)amino]-1H,5H,6H,7H-pyrrolo[3,2-c]pyridin-4-one

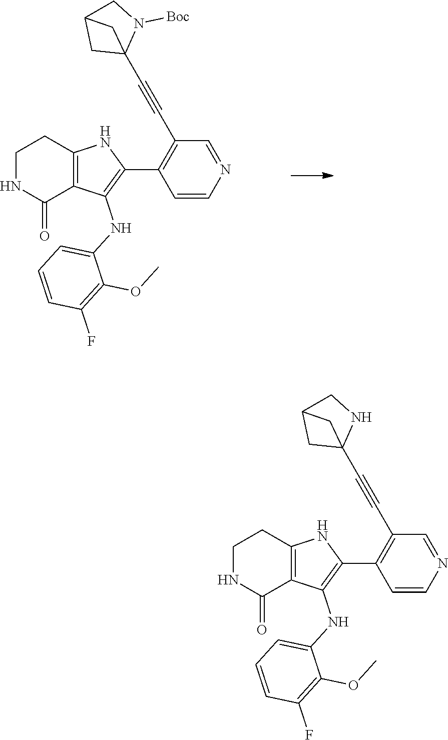 [SMILES: COc1c(F)cccc1Nc1c(-c2ccncc2C#CC23CC(CN2C(=O)OC(C)(C)C)C3)[nH]c2c1C(=O)NCC2 ; COc1c(F)cccc1Nc1c(-c2ccncc2C#CC23CC(CN2)C3)[nH]c2c1C(=O)NCC2]

To a stirred solution of 2,6-Dimethylpyridine (480 mg, 4.47 mmol, 25.00 equiv) and tert-butyl 1-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[2.1.1]hexane-2-carboxylate (100 mg, 0.18 mmol, 1.00 equiv) in DCM (4.00 mL) was added TMSOTf (0.20 mL, 0.90 mmol, 5.00 equiv) dropwise at 0° C. under nitrogen atmosphere. The resulting mixture was stirred for 4 h at room temperature under nitrogen atmosphere. The resulting mixture was concentrated under reduced pressure. The residue was purified by silica gel column chromatography, eluted with CH₂Cl₂/MeOH (10:1) to afford 2-[3-(2-{2-azabicyclo[2.1.1]hexan-1-yl}ethynyl)pyridin-4-yl]-3-[(3-fluoro-2-methoxyphenyl)amino]-1H,5H,6H,7H-pyrrolo[3,2-c]pyridin-4-one (80 mg, 97.51%) as a yellow solid.

LC-MS: (M+H)⁺ found: 458.05

### 174.3. Synthesis of 3-[(3-fluoro-2-methoxyphenyl)amino]-2-(3-{2-[2-(prop-2-enoyl)-2-azabicyclo[2.1.1]hexan-1-yl]ethynyl}pyridin-4-yl)-1H,5H,6H,7H-pyrrolo[3,2-c]pyridin-4-one

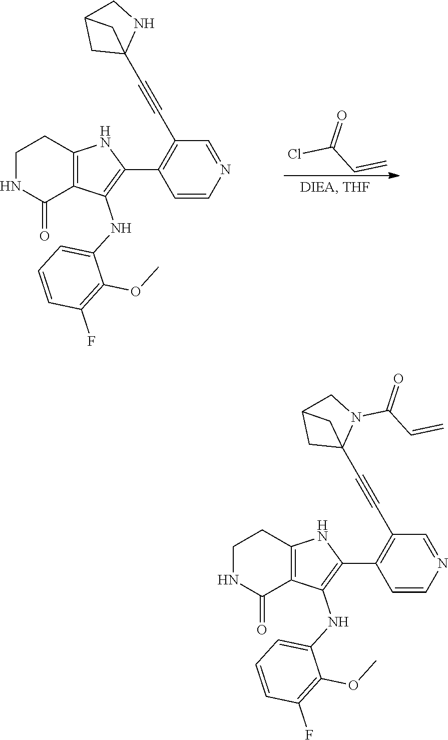

To a stirred mixture of 2-[3-(2-{2-azabicyclo[2.1.1]hexan-1-yl}ethynyl)pyridin-4-yl]-3-[(3-fluoro-2-methoxyphenyl)amino]-1H,5H,6H,7H-pyrrolo[3,2-c]pyridin-4-one (70 mg, 0.15 mmol, 1.00 equiv) in THF (0.50 mL) and DIEA (39 mg, 0.30 mmol, 2.00 equiv) was added acryloyl chloride (13 mg, 0.15 mmol, 1.00 equiv) dropwise at 0° C. under nitrogen atmosphere. The resulting mixture was stirred for 2 h at room temperature under nitrogen atmosphere. The resulting mixture was concentrated under reduced pressure. The residue was purified by silica gel column chromatography, eluted with CH₂Cl₂/MeOH (20:1) to afford crude product. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 34% B to 58% B in 10 min, 58% B; Wave Length: 254/220 nm; RT1(min): 7.53; Number Of Runs: 0) to afford 3-[(3-fluoro-2-methoxyphenyl)amino]-2-(3-{2-[2-(prop-2-enoyl)-2-azabicyclo[2.1.1]hexan-1-yl]ethynyl}pyridin-4-yl)-1H,5H,6H,7H-pyrrolo[3,2-c]pyridin-4-one (6.1 mg, 7.72%) as a yellow solid.

LC-MS: (M+H)⁺ found: 512.05

¹H NMR (400 MHz, Chloroform-d) δ 11.55 (s, 1H), 8.61 (s, 1H), 8.12 (s, 1H), 7.80 (s, 1H), 7.48 (s, 1H), 6.61-6.57 (m, 1H), 6.55-6.40 (m, 3H), 6.10 (d, 1H), 5.86-5.76 (m, 1H), 5.20 (s, 1H), 4.10 (s, 3H), 3.68 (s, 2H), 3.61-3.42 (m, 2H), 3.08 (t, 2H), 2.96 (d, 1H), 2.39 (d, 2H), 2.00-1.91 (m, 2H).

### Example 175. 3-[(3-chloro-2-methoxyphenyl)amino]-2-[3-(3-fluoro-3-methylbut-1-yn-1-yl)pyridin-4-yl]-1H,5H,6H,7H-pyrrolo[3,2-c]pyridin-4-one (compound 489)

### 175.1. Synthesis of 3-[(3-chloro-2-methoxyphenyl)amino]-2-[3-(3-hydroxy-3-methylbut-1-yn-1-yl)pyridin-4-yl]-1H,5H,6H,7H-pyrrolo[3,2-c]pyridin-4-one

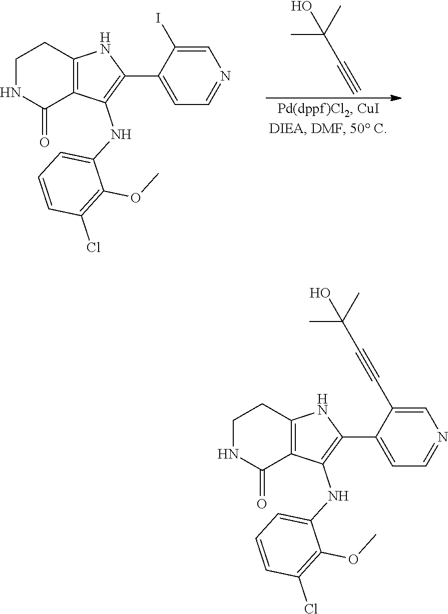

To a stirred solution of 3-[(3-chloro-2-methoxyphenyl)amino]-2-(3-iodopyridin-4-yl)-1H,5H,6H,7H-pyrrolo[3,2-c]pyridin-4-one (100.00 mg, 0.20 mmol, 1.00 equiv.) and 2-methyl-3-butyn-2-ol (85.02 mg, 1.01 mmol, 5.00 equiv.) in DMF (2 mL) were added Pd(dppf)Cl₂·CH₂Cl₂ (82.33 mg, 0.10 mmol, 0.50 equiv.) and DIEA (0.50 mL, 2.87 mmol, 14.35 equiv.) dropwise at room temperature under Argon atmosphere. The resulting mixture was stirred for 2 h at 50° C. under Argon atmosphere. The residue was purified by reverse flash chromatography with the following conditions (column: C18 silica gel; mobile phase, ACN in water, 10% to 50% gradient in 10 min; detector, UV 254 nm) to afford 3-[(3-chloro-2-methoxyphenyl)amino]-2-[3-(3-hydroxy-3-methylbut-1-yn-1-yl)pyridin-4-yl]-1H,5H,6H,7H-pyrrolo[3,2-c]pyridin-4-one (85.00 mg, 94.44%) as a yellow solid.

LC-MS: (M+H)⁺ found 451.0.

### 175.2. Synthesis of 3-[(3-chloro-2-methoxyphenyl)amino]-2-[3-(3-fluoro-3-methylbut-1-yn-1-yl)pyridin-4-yl]-1H,5H,6H,7H-pyrrolo[3,2-c]pyridin-4-one

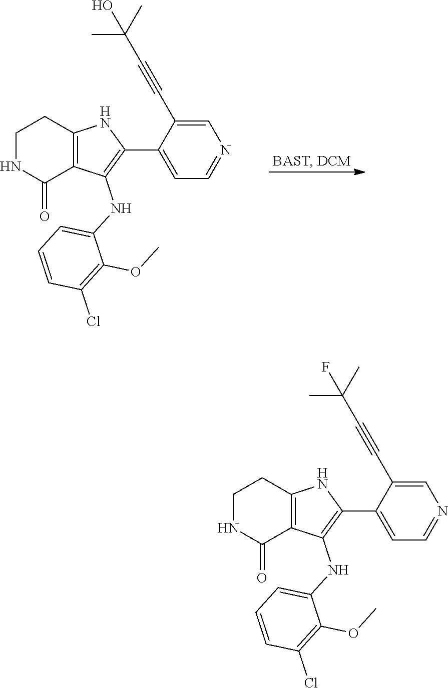 [SMILES: COc1c(Cl)cccc1Nc1c(-c2ccncc2C#CC(C)(C)O)[nH]c2c1C(=O)NCC2 ; COc1c(Cl)cccc1Nc1c(-c2ccncc2C#CC(C)(C)F)[nH]c2c1C(=O)NCC2]

To a stirred solution of 3-[(3-chloro-2-methoxyphenyl)amino]-2-[3-(3-hydroxy-3-methylbut-1-yn-1-yl)pyridin-4-yl]-1H,5H,6H,7H-pyrrolo[3,2-c]pyridin-4-one (66.00 mg, 0.15 mmol, 1.00 equiv.) in DCM (5.00 mL) were added BAST (64.76 mg, 0.29 mmol, 2.00 equiv.) dropwise at 0° C. under N₂ atmosphere. The resulting mixture was stirred for 1 h at 0° C. under N₂ atmosphere. The resulting mixture was concentrated under vacuum and dissolved in DMF. The crude product (60.00 mg) was purified by Prep-HPLC with the following conditions (Column: Xselect CSH F-Phenyl OBD column, 19*250 mm, 5¦Ìm; Mobile Phase A: Water (0.05% FA), Mobile Phase B: ACN; Flow rate: 25 mL/min; Gradient: 35% B to 43% B in 8 min, 43% B; Wave Length: 254 nm; RT1(min): 7.83; Number Of Runs: 0) to afford 3-[(3-chloro-2-methoxyphenyl)amino]-2-[3-(3-fluoro-3-methylbut-1-yn-1-yl)pyridin-4-yl]-1H,5H,6H,7H-pyrrolo[3,2-c]pyridin-4-one (12.10 mg, 17.85%) as an orange solid.

LC-MS: (M+H)⁺ found 453.0.

¹H NMR (400 MHz, DMSO-d6) δ 11.60 (s, 1H), 8.69 (s, 1H), 8.46 (d, J=5.5 Hz, 1H), 7.58-7.30 (m, 2H), 7.17 (s, 1H), 6.64 (d, J=4.9 Hz, 2H), 6.08 (h, J=4.2 Hz, 1H), 4.2 (s, 3H), 3.44 (t, J=6.8 Hz, 2H), 2.85 (t, J=6.7 Hz, 2H), 1.70 (d, J=20.6 Hz, 6H).

### Example 176. 3-[(3-chloro-2-methoxyphenyl)amino]-2-(3-[2-[(3R)-4-methylmorpholin-3-yl]ethynyl] pyridin-4-yl)-1H,5H,6H,7H-pyrrolo[3,2-c]pyridin-4-one (compound 493)

### 176.1. Synthesis of tert-butyl 3-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]morpholine-4-carboxylate

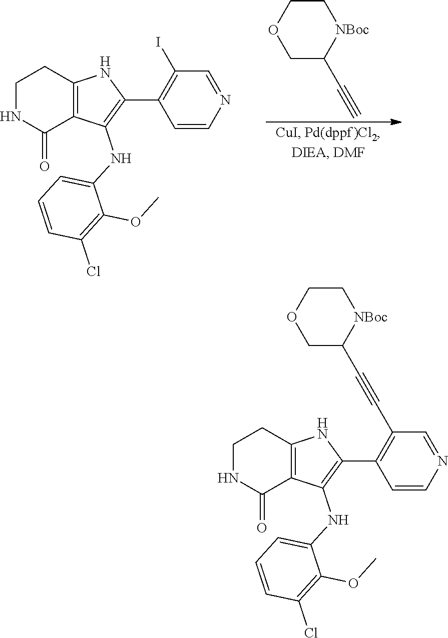 [SMILES: COc1c(Cl)cccc1Nc1c(-c2ccncc2I)[nH]c2c1C(=O)NCC2 ; C#CC1COCCN1C(=O)OC(C)(C)C ; COc1c(Cl)cccc1Nc1c(-c2ccncc2C#CC2COCCN2C(=O)OC(C)(C)C)[nH]c2c1C(=O)NCC2]

Into a 40-mL sealed tube, was placed 3-[(3-chloro-2-methoxyphenyl)amino]-2-(3-iodopyridin-4-yl)-1H,5H,6H,7H-pyrrolo[3,2-c]pyridin-4-one (200.00 mg, 0.40 mmol, 1.00 equiv.), DMF (5 mL), CuI (39.00 mg, 0.20 mmol, 0.50 equiv.), Pd(dppf)Cl₂ (148.00 mg, 0.20 mmol, 0.50 equiv.), DIEA (1 mL), tert-butyl 3-ethynylmorpholine-4-carboxylate (427.00 mg, 2.02 mmol, 5.00 equiv.). The resulting solution was stirred for 3 h at 50° C. The reaction was monitored by LCMS. The resulting mixture was filtered, the filter cake was washed with DMF (2×1 mL). The filtrate was concentrated under reduced pressure. The residue was purified by reverse flash chromatography with the following conditions (column: C18 silica gel; mobile phase, MeOH in water, 10% to 50% gradient in 10 min; detector, UV 254 nm) to afford (200.00 mg, 70.18%) of tert-butyl 3-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]morpholine-4-carboxylate as a yellow solid.

LC-MS: (M+H)⁺ found 578.0.

### 176.2. Synthesis of 3-[(3-chloro-2-methoxyphenyl)amino]-2-[3-[2-(morpholin-3-yl)ethynyl]pyridin-4-yl]-1H,5H,6H,7H-pyrrolo[3,2-c]pyridine-4-one

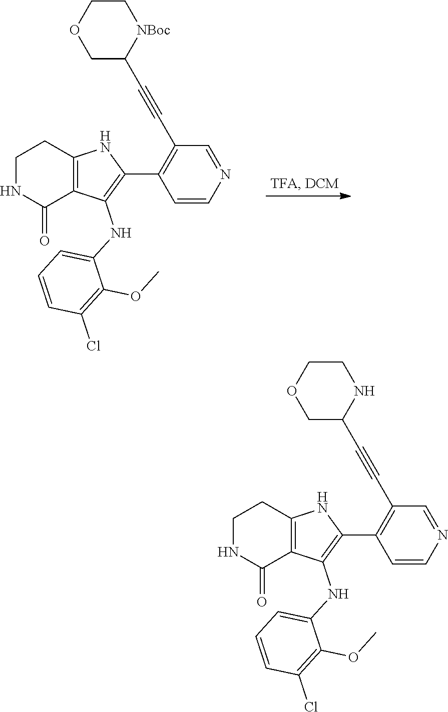 [SMILES: COc1c(Cl)cccc1Nc1c(-c2ccncc2C#CC2COCCN2C(=O)OC(C)(C)C)[nH]c2c1C(=O)NCC2 ; COc1c(Cl)cccc1Nc1c(-c2ccncc2C#CC2COCCN2)[nH]c2c1C(=O)NCC2]

To a stirred mixture of tert-butyl 3-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]morpholine-4-carboxylate (300.00 mg, 0.52 mmol, 1.00 equiv.) in DCM (5 mL) was added TFA (5 mL) at room temperature under nitrogen atmosphere. The resulting mixture was stirred for 1 h and concentrated under reduced pressure to afford 3-[(3-chloro-2-methoxyphenyl)amino]-2-[3-[2-(morpholin-3-yl)ethynyl]pyridin-4-yl]-1H,5H,6H,7H-pyrrolo[3,2-c]pyridine-4-one (220.00 mg, 70.96%) as an orange solid. The crude product was used in the next step directly without further purification.

LC-MS: (M+H)⁺ found 478.0.

### 176.3. Synthesis of afford 3-[(3-chloro-2-methoxyphenyl)amino]-2-[3-[2-(4-methylmorpholin-3-yl)ethynyl]pyridin-4-yl]-1H,5H,6H, 7H-pyrrolo [3,2-c]pyridin-4-one

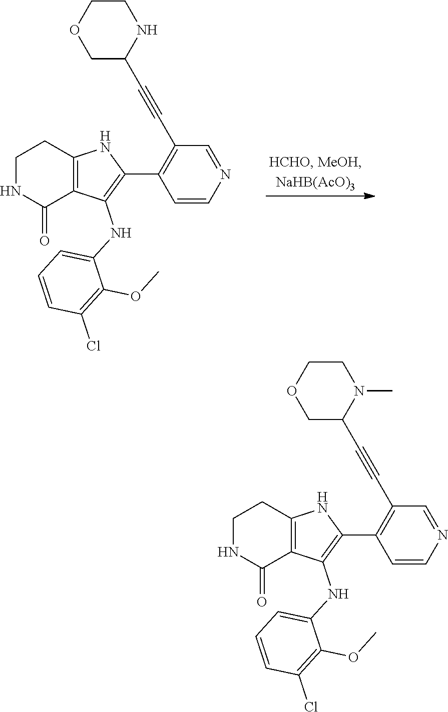 [SMILES: COc1c(Cl)cccc1Nc1c(-c2ccncc2C#CC2COCCN2)[nH]c2c1C(=O)NCC2 ; COc1c(Cl)cccc1Nc1c(-c2ccncc2C#CC2COCCN2C)[nH]c2c1C(=O)NCC2]

Into a 20-mL vial, was placed 3-[(3-chloro-2-methoxyphenyl)amino]-2-[3-[2-(morpholin-3-yl)ethynyl]pyridin-4-yl]-1H,5H,6H,7H-pyrrolo[3,2-c]pyridin-4-one (125.00 mg, 0.26 mmol, 1.00 equiv.), MeOH (4 mL), HCHO (16.00 mg, 0.52 mmol, 2.00 equiv.). The resulting solution was stirred for 30 min. This was followed by the addition of NaBH(AcO)₃ (111.00 mg, 0.52 mmol, 2.00 equiv.). The resulting solution was allowed to react, with stirring, for an additional 2 h at room temperature. The reaction was monitored by LCMS. The resulting mixture was concentrated under reduced pressure. The crude product (100.00 mg) was purified by Prep-HPLC with the following conditions (Column: XBridge Prep Amide OBD Column, 19*150 mm, 5¦Ìm 13 nm; Mobile Phase A: Water (10 mmol/L NH₄HCO₃), Mobile Phase B: ACN; Flow rate: 25 mL/min; Gradient: 32% B to 33% B in 12 min, 33% B; Wave Length: 254 nm; RT1(min): 11.48; Number Of Runs: 0) to afford 3-[(3-chloro-2-methoxyphenyl)amino]-2-[3-[2-(4-methylmorpholin-3-yl)ethynyl]pyridin-4-yl]-1H,5H,6H, 7H-pyrrolo [3,2-c]pyridin-4-one (45.00 mg, 34.90%) as a light yellow solid.

LC-MS: (M+H)⁺ found 492.0.

### 176.4. Synthesis of 3-[(3-chloro-2-methoxyphenyl)amino]-2-(3-[2-[(3R)-4-methylmorpholin-3-yl]ethynyl] pyridin-4-yl)-1H,5H,6H,7H-pyrrolo[3,2-c]pyridin-4-one

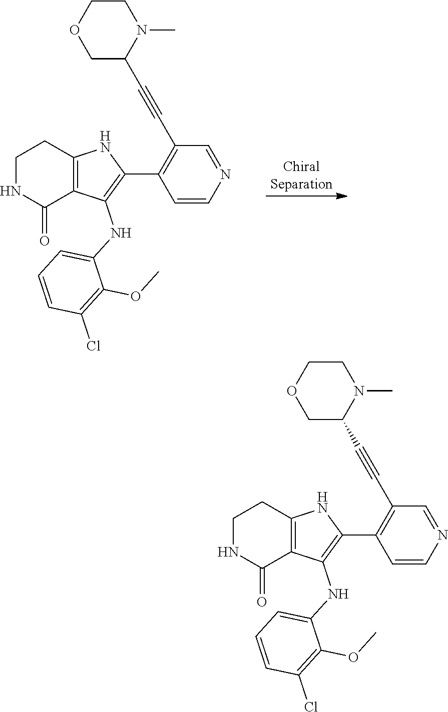 [SMILES: COc1c(Cl)cccc1Nc1c(-c2ccncc2C#CC2COCCN2C)[nH]c2c1C(=O)NCC2 ; COc1c(Cl)cccc1Nc1c(-c2ccncc2C#C[C@@H]2COCCN2C)[nH]c2c1C(=O)NCC2]

The crude product (45.00 mg) was purified by Prep-CHIRAL-HPLC with the following conditions (Column: CHIRALPAK IG, 2*25 cm, 5 μm; Mobile Phase A: Hex (0.5% 2M NH₃—MeOH)—HPLC, Mobile Phase B: EtOH—HPLC; Flow rate: 20 mL/min; Gradient: 50% B to 50% B in 27 min; Wave Length: 254/220 nm; RT1(min): 18.4; RT2(min): 22.98; Sample Solvent: EtOH—HPLC; Injection Volume: 0.4 mL; Number Of Runs: 20) to afford 3-[(3-chloro-2-methoxyphenyl)amino]-2-(3-[2-[(3R)-4-methylmorpholin-3-yl]ethynyl] pyridin-4-yl)-1H,5H,6H,7H-pyrrolo[3,2-c]pyridin-4-one (13.70 mg, 30.38%) as a light yellow solid.

LC-MS: (M+H)⁺ found 492.0.

¹H NMR (400 MHz, DMSO-d6) δ 11.45 (s, 1H), 8.61 (s, 1H), 8.38 (d, J=5.3 Hz, 1H), 7.36 (d, J=5.3 Hz, 1H), 7.27 (s, 1H), 7.10 (t, J=2.6 Hz, 1H), 6.68-6.57 (m, 2H), 6.16 (dd, J=7.4, 2.3 Hz, 1H), 3.84 (s, 3H), 3.74 (d, J=3.6 Hz, 2H), 3.69-3.53 (m, 3H), 3.50 (s, 1H), 3.40 (td, J=6.9, 2.6 Hz, 2H), 2.82 (t, J=6.8 Hz, 2H), 2.31-2.23 (m, 1H), 2.24 (s, 3H).

### Example 177. 3-[(3-chloro-2-methoxyphenyl)amino]-2-(3-[2-[(3S)-4-methylmorpholin-3-yl]ethynyl] pyridin-4-yl)-1H,5H,6H,7H-pyrrolo[3,2-c]pyridin-4-one (compound 494)

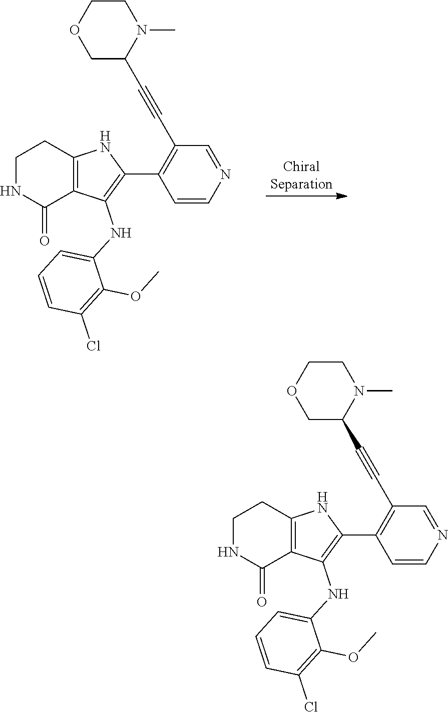 [SMILES: COc1c(Cl)cccc1Nc1c(-c2ccncc2C#CC2COCCN2C)[nH]c2c1C(=O)NCC2 ; COc1c(Cl)cccc1Nc1c(-c2ccncc2C#C[C@H]2COCCN2C)[nH]c2c1C(=O)NCC2]

The crude product (45.00 mg) was purified by Prep-CHIRAL-HPLC with the following conditions (Column: CHIRALPAK IG, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 50% B to 50% B in 27 min; Wave Length: 254/220 nm; RT1(min): 18.4; RT2(min): 22.98; Sample Solvent: EtOH—HPLC; Injection Volume: 0.4 mL; Number Of Runs: 20) to afford 3-[(3-chloro-2-methoxyphenyl)amino]-2-(3-[2-[(3S)-4-methylmorpholin-3-yl]ethynyl] pyridin-4-yl)-1H,5H,6H,7H-pyrrolo[3,2-c]pyridin-4-one (15.00 mg, 33.17%) as a light yellow solid.

LC-MS: (M+H)⁺ found 492.0.

¹H NMR (400 MHz, DMSO-d6) δ 11.44 (s, 1H), 8.61 (s, 1H), 8.38 (d, J=5.3 Hz, 1H), 7.36 (d, J=5.3 Hz, 1H), 7.27 (s, 1H), 7.10 (d, J=2.7 Hz, 1H), 6.68-6.57 (m, 2H), 6.16 (dd, J=7.4, 2.3 Hz, 1H), 3.84 (s, 3H), 3.74 (d, J=3.6 Hz, 2H), 3.69-3.52 (m, 3H), 3.50 (s, 1H), 3.40 (td, J=7.0, 2.6 Hz, 2H), 2.82 (t, J=6.8 Hz, 2H), 2.31-2.19 (m, 1H), 2.24 (s, 3H).

### Example 178. 3-[(3-chloro-2-methoxyphenyl)amino]-2-(3-{2-[(3R)-4-(prop-2-enoyl)morpholin-3-yl]ethynyl}pyridin-4-yl)-1H,5H,6H,7H-pyrrolo[3,2-c]pyridin-4-one (compound 514)

### 178.1. Synthesis of tert-butyl 3-ethynylmorpholine-4-carboxylate

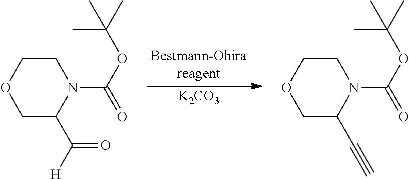

To a stirred solution of tert-butyl 3-formylmorpholine-4-carboxylate (1.10 g, 5.11 mmol, 1.00 equiv.) in MeOH (12 mL) were added K₂CO₃ (1.42 g, 10.22 mmol, 2.00 equiv.) in portions at 0° C. under nitrogen atmosphere. To the above mixture was added dimethyl (1-diazo-2-oxopropyl)phosphonate (1.18 g, 6.13 mmol, 1.20 equiv.) dropwise at 0° C. The resulting mixture was stirred for additional 1 h at room temperature. Desired product could be detected by TLC. The resulting mixture was concentrated under vacuum. The residue was purified by silica gel column chromatography, eluted with PE/EA (10:1) to afford tert-butyl 3-ethynylmorpholine-4-carboxylate (900.00 mg, 83.36%) as a colorless oil.

### 178.2. Synthesis of tert-butyl 3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]morpholine-4-carboxylate

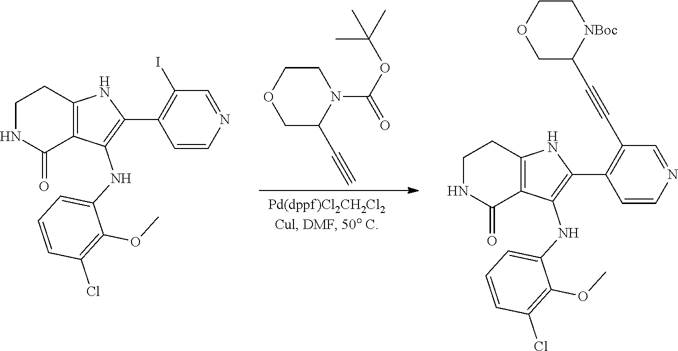 [SMILES: COc1c(Cl)cccc1Nc1c(-c2ccncc2I)[nH]c2c1C(=O)NCC2 ; C#CC1COCCN1C(=O)OC(C)(C)C ; COc1c(Cl)cccc1Nc1c(-c2ccncc2C#CC2COCCN2C(=O)OC(C)(C)C)[nH]c2c1C(=O)NCC2]

To a stirred solution of tert-butyl 3-ethynylmorpholine-4-carboxylate (281 mg, 1.33 mmol, 3.00 equiv.) and 3-[(3-chloro-2-methoxyphenyl)amino]-2-(3-iodopyridin-4-yl)-1H,5H,6H,7H-pyrrolo[3,2-c]pyridin-4-one (220.00 mg, 0.44 mmol, 1.00 equiv.) in DMF (3 mL) were added Pd(dppf)Cl₂·CH₂Cl₂ (181.00 mg, 0.22 mmol, 0.50 equiv.) and CuI (42.00 mg, 0.22 mmol, 0.50 equiv.). The resulting mixture was stirred for 1.5 h at 50° C. under nitrogen atmosphere. The residue was purified by reverse flash chromatography with the following conditions (column: C18 silica gel; mobile phase, ACN in water, 10% to 50% gradient in 30 min; detector, UV 254 nm) to afford tert-butyl 3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]morpholine-4-carboxylate (130.00s mg, 50.57%) as a yellow solid.

LC-MS: (M+H)⁺ found 578.1.

### 178.3. Synthesis of 3-[(3-chloro-2-methoxyphenyl)amino]-2-{3-[2-(morpholin-3-yl)ethynyl]pyridin-4-yl}-1H,5H,6H,7H-pyrrolo[3,2-c]pyridin-4-one

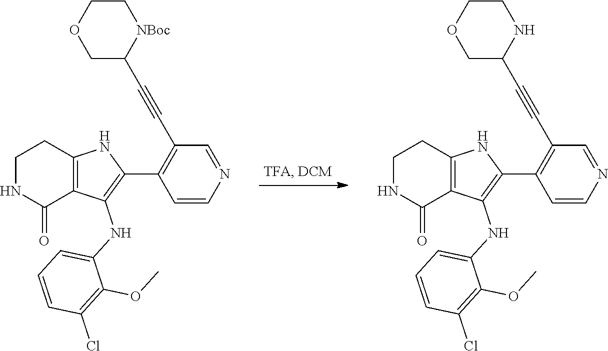 [SMILES: COc1c(Cl)cccc1Nc1c(-c2ccncc2C#CC2COCCN2C(=O)OC(C)(C)C)[nH]c2c1C(=O)NCC2 ; COc1c(Cl)cccc1Nc1c(-c2ccncc2C#CC2COCCN2)[nH]c2c1C(=O)NCC2]

To a stirred solution of tert-butyl 3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]morpholine-4-carboxylate (130.00 mg, 0.23 mmol, 1.00 equiv.) in DCM (2 mL) was added TFA (1 mL) dropwise at 0° C. under nitrogen atmosphere. The resulting mixture was stirred for 1 h at room temperature under nitrogen atmosphere. The resulting mixture was concentrated under reduced pressure. The crude product 3-[(3-chloro-2-methoxyphenyl)amino]-2-{3-[2-(morpholin-3-yl)ethynyl]pyridin-4-yl}-1H,5H,6H,7H-pyrrolo[3,2-c]pyridin-4-one (119.00 mg, 99.64%) was obtained and used in the next step directly without further purification.

LC-MS: (M+H)⁺ found 478.0.

### 178.4. Synthesis of 3-[(3-chloro-2-methoxyphenyl)amino]-2-(3-{2-[4-(prop-2-enoyl)morpholin-3-yl]ethynyl}pyridin-4-yl)-1H,5H,6H,7H-pyrrolo[3,2-c]pyridin-4-one

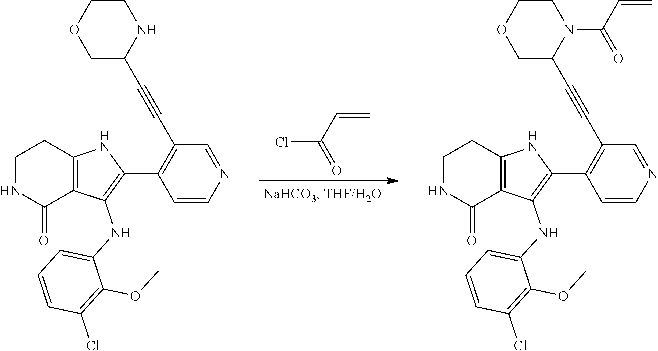

To a stirred solution of 3-[(3-chloro-2-methoxyphenyl)amino]-2-{3-[2-(morpholin-3-yl)ethynyl]pyridin-4-yl}-1H,5H,6H,7H-pyrrolo[3,2-c]pyridin-4-one (120.00 mg, 0.19 mmol, 1.00 equiv.) in THF (2 mL) were added NaHCO₃aq. (2 mL) in one portion at 0° C. under nitrogen atmosphere to basified the mixture. To the above mixture was added acryloyl chloride (15.00 mg, 0.17 mmol, 0.90 equiv.) dropwise at 0° C. The resulting mixture was stirred for additional 1 h at 0° C. The resulting mixture was extracted with CH₂Cl₂:MeOH=10:1 (3×20 mL). The combined organic layers were dried over anhydrous Na₂SO₄. The resulting mixture was concentrated under vacuum and dissolved in DMSO. The crude product (100.00 mg) was purified by Prep-HPLC with the following conditions (Column: XBridge Prep C18 OBD Column, 19*150 mm, 5 μm; Mobile Phase A: Water (10 mmol/L NH₄HCO₃), Mobile Phase B: ACN; Flow rate: 60 mL/min; Gradient: 20% B to 42% B in 10 min, 42% B; Wave Length: 254/220 nm; RT1(min): 9.67; Number Of Runs: 0) to afford 3-[(3-chloro-2-methoxyphenyl)amino]-2-(3-{2-[4-(prop-2-enoyl)morpholin-3-yl]ethynyl}pyridin-4-yl)-1H,5H,6H,7H-pyrrolo[3,2-c]pyridin-4-one (50.00 mg, 37.43%) as a yellow solid.

LC-MS: (M+H)⁺ found 531.9.

### 178.5. Synthesis of 3-[(3-chloro-2-methoxyphenyl)amino]-2-(3-{2-[(3R)-4-(prop-2-enoyl)morpholin-3-yl]ethynyl}pyridin-4-yl)-1H,5H,6H,7H-pyrrolo[3,2-c]pyridin-4-one

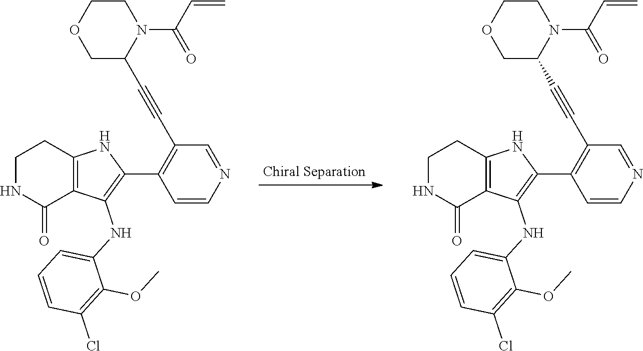 [SMILES: C=CC(=O)N1CCOCC1C#Cc1cnccc1-c1[nH]c2c(c1Nc1cccc(Cl)c1OC)C(=O)NCC2 ; C=CC(=O)N1CCOC[C@H]1C#Cc1cnccc1-c1[nH]c2c(c1Nc1cccc(Cl)c1OC)C(=O)NCC2]

The 3-[(3-chloro-2-methoxyphenyl)amino]-2-(3-{2-[4-(prop-2-enoyl)morpholin-3-yl]ethynyl}pyridin-4-yl)-1H,5H,6H,7H-pyrrolo[3,2-c]pyridin-4-one (50.00 mg) was purified by CHIRAL-HPLC with the following conditions (Column: CHIRALPAK IG-3, 4.6*50 mm, 3 um; Mobile Phase A: Hex (0.1% DEA):EtOH=50:50; Flow rate: 1 mL/min; Gradient: 0% B to 0% B; Injection Volume: 5 ul mL) to afford 3-[(3-chloro-2-methoxyphenyl)amino]-2-(3-{2-[(3R)-4-(prop-2-enoyl)morpholin-3-yl]ethynyl}pyridin-4-yl)-1H,5H,6H,7H-pyrrolo[3,2-c]pyridin-4-one (19.20 mg, 38.40%) as a light yellow solid.

LC-MS: (M+H)⁺ found 532.0.

¹H NMR (400 MHz, Chloroform-d) δ 10.53 (s, 1H), 8.82-7.99 (m, 2H), 6.79 (d, J=7.9 Hz, 1H), 6.61 (m, 2H), 6.45-6.40 (m, 1H), 6.18 (d, J=8.0 Hz, 1H), 5.86 (d, J=10.4 Hz, 1H), 5.50 (s, 1H), 5.29 (s, 1H), 4.22 (d, J=11.6 Hz, 1H), 4.07 (s, 4H), 3.99-3.19 (m, 8H), 3.13 (brs, 2H).

### Example 179. 3-[(3-chloro-2-methoxyphenyl)amino]-2-(3-{2-[(3S)-4-(prop-2-enoyl)morpholin-3-yl]ethynyl} pyridine-4-yl)-1H,5H,6H,7H-pyrrolo[3,2-c]pyridin-4-one (compound 515)

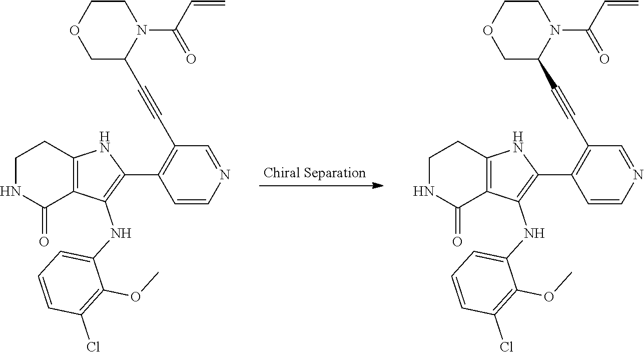 [SMILES: C=CC(=O)N1CCOCC1C#Cc1cnccc1-c1[nH]c2c(c1Nc1cccc(Cl)c1OC)C(=O)NCC2 ; C=CC(=O)N1CCOC[C@@H]1C#Cc1cnccc1-c1[nH]c2c(c1Nc1cccc(Cl)c1OC)C(=O)NCC2]

The 3-[(3-chloro-2-methoxyphenyl)amino]-2-(3-{2-[4-(prop-2-enoyl)morpholin-3-yl]ethynyl}pyridin-4-yl)-1H,5H,6H,7H-pyrrolo[3,2-c]pyridin-4-one (50.00 mg, 0.09 mmol, 1.00 equiv.) was purified by CHRIAL-HPLC with the following conditions (Column: CHIRALPAK IG-3, 4.6*50 mm, 3 um; Mobile Phase A: Hex (0.1% DEA):EtOH=50:50; Flow rate: 1 mL/min; Gradient: 0% B to 0% B; Injection Volume: 5 ul mL) to afford 3-[(3-chloro-2-methoxyphenyl)amino]-2-(3-{2-[(3S)-4-(prop-2-enoyl)morpholin-3-yl]ethynyl} pyridine-4-yl)-1H,5H,6H,7H-pyrrolo[3,2-c]pyridin-4-one (16.90 mg, 33.80%) as a light yellow solid.

LC-MS: (M+H)⁺ found 532.0.

¹H NMR (400 MHz, Chloroform-d) δ 10.50 (s, 1H), 8.62-7.90 (m, 2H), 6.77 (d, J=7.9 Hz, 1H), 6.70-6.50 (m, 2H), 6.44-6.39 (m, 1H), 6.19 (d, J=8.0 Hz, 1H), 5.86 (d, J=10.3 Hz, 1H), 5.52 (s, 1H), 5.28 (s, 1H), 4.22 (d, J=11.6 Hz, 1H), 4.07 (s, 4H), 3.87-3.14 (m, 8H), 3.11 (brs, 2H).

### Example 180. 3-[(3-chloro-2-methoxyphenyl)amino]-2-{3-[2-(1-fluorocyclopropyl)ethynyl]pyridin-4-yl}-1H,5H,6H,7H-pyrrolo[3,2-c]pyridin-4-one (compound 545)

### 180.1. Synthesis of 3-((3-chloro-2-methoxyphenyl)amino)-2-(3-((1-hydroxycyclopropyl)ethynyl) pyridin-4-yl)-1,5,6,7-tetrahydro-4H-pyrrolo[3,2-c]pyridin-4-one

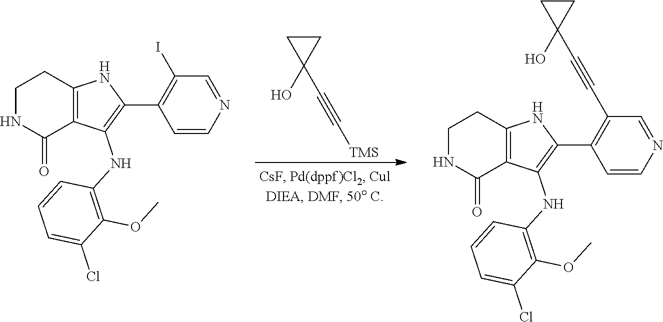 [SMILES: COc1c(Cl)cccc1Nc1c(-c2ccncc2I)[nH]c2c1C(=O)NCC2 ; C[Si](C)(C)C#CC1(O)CC1 ; COc1c(Cl)cccc1Nc1c(-c2ccncc2C#CC2(O)CC2)[nH]c2c1C(=O)NCC2]

Into a sealed tube were added 3-[(3-chloro-2-methoxyphenyl)amino]-2-(3-iodopyridin-4-yl)-1H,5H,6H,7H-pyrrolo[3,2-c]pyridin-4-one (100.00 mg, 0.20 mmol, 1.00 equiv.), CuI (19.00 mg, 0.10 mmol, 0.50 equiv.), CsF (61.00 mg, 0.40 mmol, 2.00 equiv.), Pd(dppf)Cl₂·CH₂Cl₂ (82.00 mg, 0.10 mmol, 0.50 equiv.), DMF (5 mL), 1-((trimethylsilyl)ethynyl)cyclopropan-1-ol (154.00 mg, 1.00 mmol, 5.00 equiv.) and DIEA (0.5 mL) at room temperature. After stirring for 2 h at 50° C. under at Argon atmosphere. The reaction was monitored by LCMS. The resulting mixture was filtered. The residue was purified by silica gel column chromatography, eluted with CH₂Cl₂/MeOH (10:1) to afford 3-((3-chloro-2-methoxyphenyl)amino)-2-(3-((1-hydroxycyclopropyl)ethynyl) pyridin-4-yl)-1,5,6,7-tetrahydro-4H-pyrrolo[3,2-c]pyridin-4-one (67.00 mg, 74.8%) as a brown solid.

LC-MS: (M+H)⁺ found 449.0.

### 180.2. Synthesis of 3-[(3-chloro-2-methoxyphenyl)amino]-2-{3-[2-(1-fluorocyclopropyl)ethynyl]pyridin-4-yl}-1H,5H,6H,7H-pyrrolo[3,2-c]pyridin-4-one

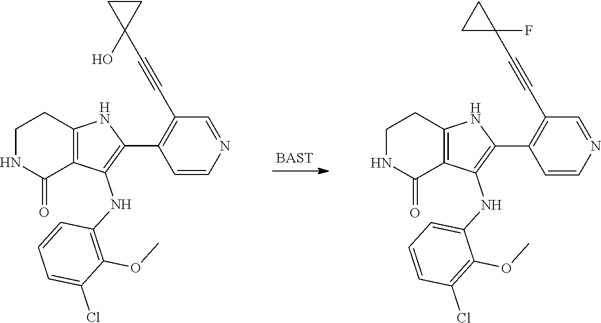 [SMILES: COc1c(Cl)cccc1Nc1c(-c2ccncc2C#CC2(O)CC2)[nH]c2c1C(=O)NCC2 ; COc1c(Cl)cccc1Nc1c(-c2ccncc2C#CC2(F)CC2)[nH]c2c1C(=O)NCC2]

To a stirred solution of 3-[(3-chloro-2-methoxyphenyl)amino]-2-{3-[2-(1-hydroxycyclopropyl)ethynyl] pyridin-4-yl}-1H,5H,6H,7H-pyrrolo[3,2-c]pyridin-4-one) (60.00 mg, 0.13 mmol, 1.00 equiv.) in CH₂Cl₂ (10 mL) were added BAST (59.00 mg, 0.26 mmol, 2.00 equiv.) dropwise at 0° C. under nitrogen atmosphere. The resulting mixture was stirred for 0.5 h at 0° C. under nitrogen atmosphere. The reaction was monitored by LCMS. The reaction was quenched with sat. sodium hyposulfite (aq.) at 0° C. The resulting mixture was extracted with CH₂Cl₂ (3×30 mL). The combined organic layers were washed with brine, dried over anhydrous Na₂SO₄. After filtration, the filtrate was concentrated under reduced pressure. The residue was purified by silica gel column chromatography, eluted with CH₂Cl₂/MeOH (10:1) to afford 3-[(3-chloro-2-methoxyphenyl)amino]-2-{3-[2-(1-fluorocyclopropyl)ethynyl]pyridin-4-yl}-1H,5H,6H,7H-pyrrolo[3,2-c]pyridin-4-one (6.20 mg, 10.29%) as a red solid.

LC-MS: (M+H)⁺ found 451.0.

¹H NMR (400 MHz, Methanol-d4) δ 8.72 (s, 1H), 8.35 (s, 1H), 7.60 (d, J=5.9 Hz, 1H), 6.75 (dd, J=8.0, 1.5 Hz, 1H), 6.66 (t, J=8.1 Hz, 1H), 6.22 (dd, J=8.2, 1.5 Hz, 1H), 3.97 (s, 3H), 3.64 (t, J=6.9 Hz, 2H), 3.01 (t, J=6.9 Hz, 2H), 1.57-1.44 (m, 2H), 1.28 (dt, J=9.1, 6.2 Hz, 2H).

### Example 181. 3-[(3-chloro-2-methoxyphenyl)amino]-2-[3-(prop-1-yn-1-yl)pyridin-4-yl]-1H,5H,6H,7H-pyrrolo[3,2-c]pyridin-4-one (compound 547)

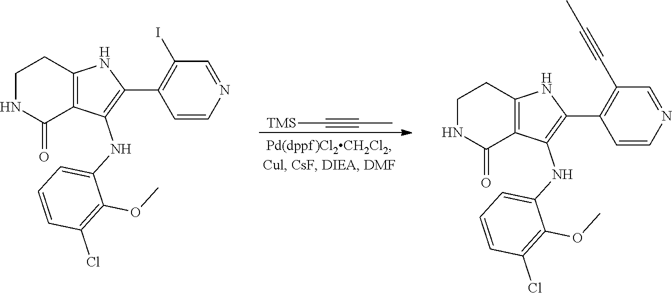

Into a sealed tube were added 3-[(3-chloro-2-methoxyphenyl)amino]-2-(3-iodopyridin-4-yl)-1H,5H,6H,7H-pyrrolo[3,2-c]pyridin-4-one (100.00 mg, 0.20 mmol, 1.00 equiv.), CuI (19.00 mg, 0.101 mmol, 0.50 equiv.), CsF (61.00 mg, 0.40 mmol, 2.00 equiv.), Pd(dppf)C12-CH₂Cl₂ (83.00 mg, 0.10 mmol, 0.50 equiv.), DMF (2 mL), trimethyl(prop-1-yn-1-yl)silane (113.00 mg, 1.00 mmol, 5.00 equiv.) and DIEA (0.50 mL) at room temperature. After stirring for 2 h at 50° C. under Argon atmosphere. The reaction was monitored by LCMS. The resulting mixture was filtered. The residue was purified by silica gel column chromatography, eluted with CH₂Cl₂/MeOH (10:1) to afford 3-[(3-chloro-2-methoxyphenyl)amino]-2-[3-(prop-1-yn-1-yl)pyridin-4-yl]-1H,5H,6H,7H-pyrrolo[3,2-c]pyridin-4-one (2.30 mg, 2.71%) as a yellow solid.

LC-MS: (M+H)⁺ found 407.0.

¹H NMR (300 MHz, Methanol-d4) δ 8.52 (s, 1H), 8.22 (s, 1H), 7.40 (s, 1H), 6.69-6.53 (m, 2H), 6.23 (dd, J=7.4, 2.4 Hz, 1H), 3.95 (s, 3H), 3.62 (t, J=6.9 Hz, 2H), 2.98 (t, J=7.0 Hz, 2H), 2.15 (s, 3H).

### Example 182. 3-[(3-chloro-2-methoxyphenyl)amino]-2-[3-[2-(1-methylcyclopropyl)ethynyl]pyridin-4-yl]-1H,5H,6H,7H-pyrrolo[3,2-c]pyridin-4-one (compound 527)

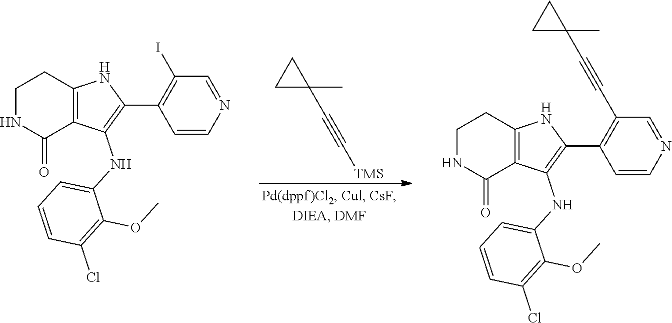

Into a sealed tube were added 3-[(3-chloro-2-methoxyphenyl)amino]-2-(3-iodopyridin-4-yl)-1H,5H,6H,7H-pyrrolo[3,2-c]pyridin-4-one (100.00 mg, 0.20 mmol, 1.00 equiv.), 5 CuI (19.00 mg, 0.10 mmol, 0.50 equiv.), CsF (61.00 mg, 0.40 mmol, 2.00 equiv.), Pd(dppf)Cl₂·CH₂Cl₂ (82.00 mg, 0.10 mmol, 0.50 equiv.), DMF (5 mL), trimethyl[2-(1-methylcyclopropyl)ethynyl]silane (154.00 mg, 1.00 mmol, 5.00 equiv.) and DIEA (0.50 mL, 2.87 mmol, 14.20 equiv.) at room temperature. After stirring for 2 h at 50° C. under Argon atmosphere, the reaction was monitored by LCMS. The resulting mixture was concentrated and the residue was purified by silica gel column chromatography, eluted with CH₂Cl₂/MeOH (10:1) to afford crude product. The crude product was purified by Prep-HPLC to afford 3-[(3-chloro-2-methoxyphenyl)amino]-2-[3-[2-(1-methylcyclopropyl)ethynyl]pyridin-4-yl]-1H,5H,6H,7H-pyrrolo[3,2-c]pyridin-4-one (10.20 mg, 11.72%) as a yellow solid.

LC-MS: (M+H)⁺ found 447.0.

¹H NMR (400 MHz, DMSO-d6): δ 11.45 (s, 1H), 8.51 (s, 1H), 8.32 (d, J=5.2 Hz, 1H), 7.28-7.27 (m, 2H), 7.09 (s, 1H), 6.65-6.59 (m, 2H), 6.09 (dd, J=7.6, 2.4 Hz, 1H), 3.83 (s, 3H), 3.43 (td, J=6.8, 2.4 Hz, 2H), 2.84 (t, J=6.8 Hz, 2H), 1.31 (s, 3H), 0.99-0.97 (m, 2H), 0.77-0.75 (m, 2H).

### Example 183. 3-[(3-chloro-2-methoxyphenyl)amino]-2-(3-{2-[(2R)-2-methyl-1-(prop-2-enoyl)azetidin-2-yl]ethynyl}pyridin-4-yl)-1H,5H,6H,7H-pyrrolo[3,2-c]pyridin-4-one (compound 551)

### 183.1. Synthesis of tert-butyl 2-formyl-2-methylazetidine-1-carboxylate

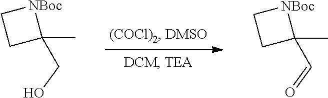

To a stirred solution of (COCl)₂ (560.00 mg, 4.41 mmol, 1.20 equiv.) in DCM (40 mL) were added DMSO (0.65 mL, 9.19 mmol, 2.50 equiv.) dropwise at −78° C. under nitrogen atmosphere. The resulting mixture was stirred for 30 min at −78° C. under nitrogen atmosphere. To the above mixture was added tert-butyl 2-(hydroxymethyl)-2-methylazetidine-1-carboxylate (740.00 mg, 3.68 mmol, 1.00 equiv.) dropwise at −78° C. The resulting mixture was stirred for additional 30 min at −78° C. Desired product could be detected by TLC. To the above mixture was added TEA (2.56 mL, 18.39 mmol, 5.00 equiv.) at −78° C. The resulting mixture was stirred for additional 30 min at 0° C. The resulting mixture was extracted with CH₂Cl₂ (3×40 mL). The combined organic layers were washed with water (3×50 mL), dried over anhydrous Na₂SO₄. After filtration, the filtrate was concentrated under reduced pressure. The crude product tert-butyl 2-formyl-2-methylazetidine-1-carboxylate (750.00 mg, 102.38%) was used in the next step directly without further purification.

### 183.2. Synthesis of tert-butyl 2-ethynyl-2-methylazetidine-1-carboxylate

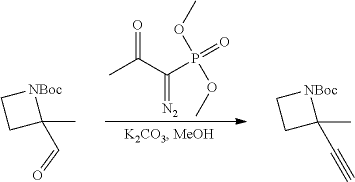

To a stirred solution of tert-butyl 2-formyl-2-methylazetidine-1-carboxylate (750.00 mg, 3.76 mmol, 1.00 equiv.) in MeOH (10 mL) were added K₂CO₃ (1.04 g, 7.53 mmol, 2.00 equiv.) and seyferth-gilbert homologation (868.00 mg, 4.52 mmol, 1.20 equiv.) dropwise at 0° C. under nitrogen atmosphere. The resulting mixture was stirred for 1 h at 0° C. under nitrogen atmosphere. Desired product could be detected by TLC. The reaction was quenched by the addition of Potassium sodium tartrate (aq.) (5 mL) at 0° C. The resulting mixture was extracted with EtOAc (3×30 mL). The combined organic layers were dried over anhydrous Na₂SO₄. After filtration, the filtrate was concentrated under reduced pressure. The residue was purified by silica gel column chromatography, eluted with PE/EA (10:1) to afford tert-butyl 2-ethynyl-2-methylazetidine-1-carboxylate (440.00 mg, 59.86%) as a colorless oil.

### 183.3. Synthesis of tert-butyl 2-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]-2-methylazetidine-1-carboxylate

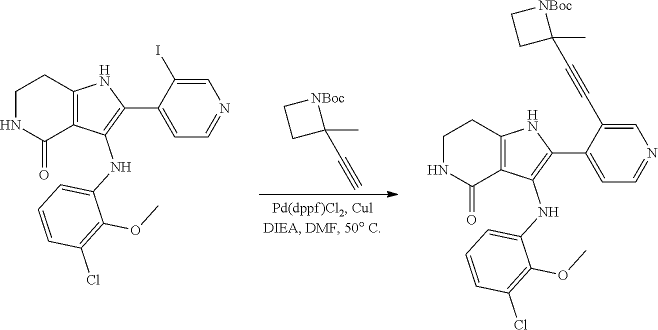 [SMILES: COc1c(Cl)cccc1Nc1c(-c2ccncc2I)[nH]c2c1C(=O)NCC2 ; C#CC1(C)CCN1C(=O)OC(C)(C)C ; COc1c(Cl)cccc1Nc1c(-c2ccncc2C#CC2(C)CCN2C(=O)OC(C)(C)C)[nH]c2c1C(=O)NCC2]

Into a 20 mL sealed tube were added 3-[(3-chloro-2-methoxyphenyl)amino]-2-(3-iodopyridin-4-yl)-1H,5H,6H,7H-pyrrolo[3,2-c]pyridin-4-one (540.00 mg, 1.09 mmol, 1.00 equiv.) and Pd(dppf)C12 CH₂Cl₂ (178.00 mg, 0.22 mmol, 0.20 equiv.) and CuI (83.00 mg, 0.44 mmol, 0.40 equiv.) and DIEA (846.00 mg, 6.55 mmol, 6.00 equiv.) and DMF (50 mL) at room temperature. To the above mixture was added tert-butyl 2-ethynyl-2-methylazetidine-1-carboxylate (640.00 mg, 3.28 mmol, 3.00 equiv.) dropwise at room temperature. The resulting mixture was stirred for 4 h at 50° C. under Argon atmosphere. The reaction was monitored by LCMS. The residue was purified by reverse flash chromatography with the following conditions (column: C18 silica gel; mobile phase, ACN in water, 40% to 60% gradient in 10 min; detector, UV 254 nm) to afford tert-butyl 2-[2-(4-[3-[(3-chloro-2-methoxyphenyl)amino]-4-oxo-1H,5H,6H,7H-pyrrolo[3,2-c]pyridin-2-yl]pyridin-3-yl)ethynyl]-2-methylazetidine-1-carboxylate (640.00 mg, 99.10%) as a yellow solid.

LC-MS: (M+H)⁺ found 562.1.

### 183.4. Synthesis of 3-[(3-chloro-2-methoxyphenyl)amino]-2-{3-[2-(2-methylazetidin-2-yl)ethynyl]pyridin-4-yl}-1H,5H,6H,7H-pyrrolo[3,2-c]pyridin-4-one

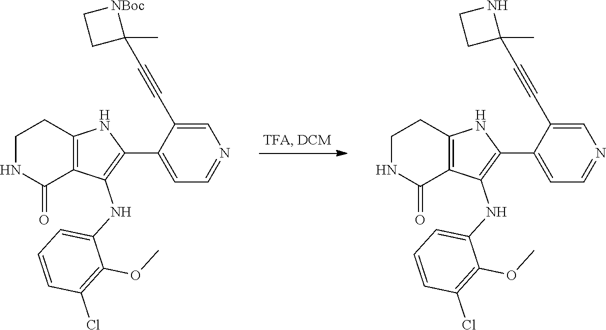 [SMILES: COc1c(Cl)cccc1Nc1c(-c2ccncc2C#CC2(C)CCN2C(=O)OC(C)(C)C)[nH]c2c1C(=O)NCC2 ; COc1c(Cl)cccc1Nc1c(-c2ccncc2C#CC2(C)CCN2)[nH]c2c1C(=O)NCC2]

To a stirred mixture of tert-butyl 2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-methylazetidine-1-carboxylate (400.00 mg, 0.71 mmol, 1.00 equiv.) in DCM (12 mL) was added TFA (4 mL) at room temperature under nitrogen atmosphere. The resulting mixture was stirred for 1 h and concentrated under reduced pressure to afford 3-[(3-chloro-2-methoxyphenyl)amino]-2-{3-[2-(2-methylazetidin-2-yl)ethynyl]pyridin-4-yl}-1H,5H,6H,7H-pyrrolo[3,2-c]pyridin-4-one (296.00 mg, 90.04%) as a red oil. The crude product was used in the next step directly without further purification.

LC-MS: (M+H)⁺ found 462.0.

### 183.5. Synthesis of 3-[(3-chloro-2-methoxyphenyl) amino]-2-(3-[2-[2-methyl-1-(prop-2-enoyl)azetidin-2-yl]ethynyl]pyridin-4-yl)-1H,5H,6H,7H-pyrrolo [3,2-c]pyridin-4-one

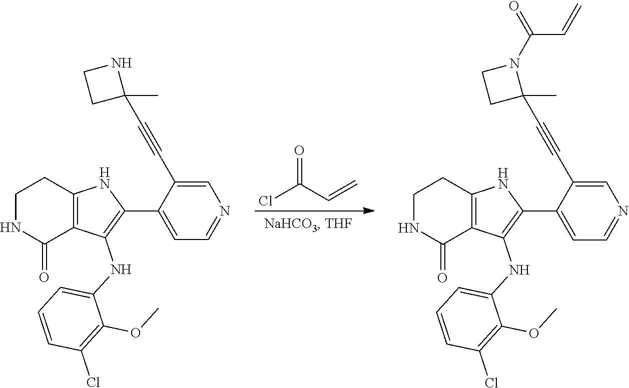

To a stirred solution of 3-[(3-chloro-2-methoxyphenyl)amino]-2-[3-[2-(2-methylazetidin-2-yl)ethynyl]pyridin-4-yl]-1H,5H,6H,7H-pyrrolo[3,2-c]pyridin-4-one (300.00 mg, 0.65 mmol, 1.00 equiv.) in NaHCO₃(sat.) (6 mL) and THF (6 mL) was added acryloyl chloride (59.00 mg, 0.65 mmol, 1.00 equiv.) dropwise at 0° C. under nitrogen atmosphere. The resulting mixture was stirred for 1 h at room temperature under nitrogen atmosphere. Desired product could be detected by LCMS. The reaction was quenched by the addition of MeOH (0.50 mL) at room temperature. The resulting mixture was diluted with water (10 mL). The aqueous layer was extracted with CH₂Cl₂ (3×10 mL). The combined organic layers were concentrated under reduced pressure. The crude product was purified by Prep-HPLC with the following conditions (Column: YMC-Actus Triart C18 ExRS, 30*250 mm, 5 μm; Mobile Phase A: Water (10 mmol/L NH₄HCO₃), Mobile Phase B: ACN; Flow rate: 60 mL/min; Gradient: 21% B to 37% B in 10 min, 37% B; Wave Length: 254/220 nm; RT1(min): 9.67; Number Of Runs: 0) to afford 3-[(3-chloro-2-methoxyphenyl) amino]-2-(3-[2-[2-methyl-1-(prop-2-enoyl)azetidin-2-yl]ethynyl]pyridin-4-yl)-1H,5H,6H,7H-pyrrolo [3,2-c]pyridin-4-one (37.00 mg, 11.04%) as a yellow solid.

LC-MS: (M+H)⁺ found 516.2.

### 183.6. Synthesis of 3-[(3-chloro-2-methoxyphenyl)amino]-2-(3-{2-[(2R)-2-methyl-1-(prop-2-enoyl)azetidin-2-yl]ethynyl}pyridin-4-yl)-1H,5H,6H,7H-pyrrolo[3,2-c]pyridin-4-one

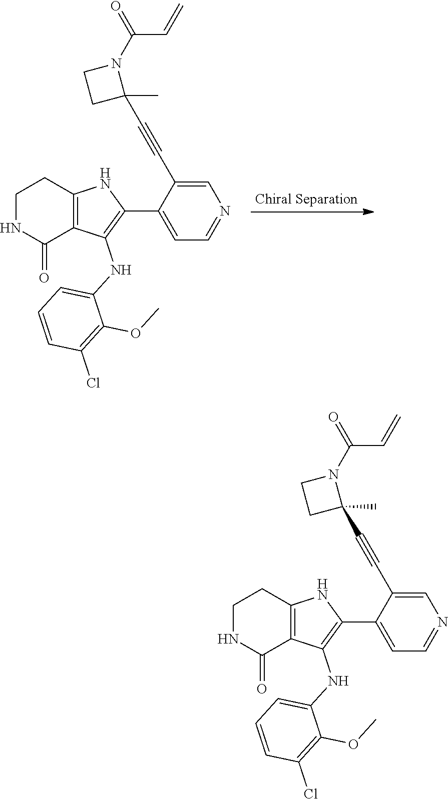 [SMILES: C=CC(=O)N1CCC1(C)C#Cc1cnccc1-c1[nH]c2c(c1Nc1cccc(Cl)c1OC)C(=O)NCC2 ; C=CC(=O)N1CC[C@]1(C)C#Cc1cnccc1-c1[nH]c2c(c1Nc1cccc(Cl)c1OC)C(=O)NCC2]

3-[(3-chloro-2-methoxyphenyl)amino]-2-(3-{2-[2-methyl-1-(prop-2-enoyl)azetidin-2-yl]ethynyl}pyridin-4-yl)-1H,5H,6H,7H-pyrrolo[3,2-c]pyridin-4-one (37.00 mg) was purified by Prep-CHIRAL-HPLC with the following conditions (Column: CHIRAL ART Amylose-SA, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 20% B to 20% B in 20 min; Wave Length: 220/254 nm; RT1(min): 12.176; RT2(min): 16.377; Sample Solvent: EtOH—HPLC; Injection Volume: 1 mL; Number Of Runs: 2) to afford 3-[(3-chloro-2-methoxyphenyl)amino]-2-(3-{2-[(2R)-2-methyl-1-(prop-2-enoyl)azetidin-2-yl]ethynyl}pyridin-4-yl)-1H,5H,6H,7H-pyrrolo[3,2-c]pyridin-4-one (18.80 mg, 49.69%) as a yellow solid.

LC-MS: (M+H)⁺ found 516.2.

¹H NMR (300 MHz, DMSO-d6) δ 11.14 (s, 1H), 8.76 (s, 1H), 8.40 (d, J=5.2 Hz, 1H), 8.34 (s, 1H), 8.01 (s, 1H), 7.16 (d, J=6.3 Hz, 2H), 6.88 (d, J=8.3 Hz, 1H), 6.77 (d, J=8.3 Hz, 1H), 6.20-5.94 (m, 2H), 5.53 (d, J=3.1 Hz, 1H), 3.73 (s, 3H), 3.36 (s, 2H), 3.11 (s, 2H), 2.83-2.66 (m, 2H), 2.08 (s, 3H), 1.69 (s, 3H).

### Example 184. 3-[(3-chloro-2-methoxyphenyl)amino]-2-(3-{2-[(2S)-2-methyl-1-(prop-2-enoyl)azetidin-2-yl]ethynyl}pyridin-4-yl)-1H,5H,6H,7H-pyrrolo[3,2-c]pyridin-4-one (compound 550)

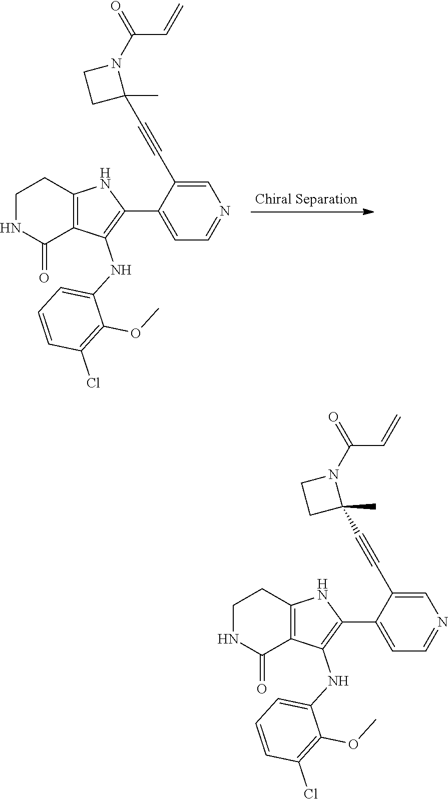 [SMILES: C=CC(=O)N1CCC1(C)C#Cc1cnccc1-c1[nH]c2c(c1Nc1cccc(Cl)c1OC)C(=O)NCC2 ; C=CC(=O)N1CC[C@@]1(C)C#Cc1cnccc1-c1[nH]c2c(c1Nc1cccc(Cl)c1OC)C(=O)NCC2]

The 3-[(3-chloro-2-methoxyphenyl)amino]-2-(3-{2-[2-methyl-1-(prop-2-enoyl)azetidin-2-yl]ethynyl}pyridin-4-yl)-1H,5H,6H,7H-pyrrolo[3,2-c]pyridin-4-one (37.00 mg) was purified by Prep-CHIRAL-HPLC with the following conditions (Column: CHIRAL ART Amylose-SA, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 20% B to 20% B in 20 min; Wave Length: 220/254 nm; RT1(min): 12.176; RT2(min): 16.377; Sample Solvent: EtOH—HPLC; Injection Volume: 1 mL; Number Of Runs: 2) to afford 3-[(3-chloro-2-methoxyphenyl)amino]-2-(3-{2-[(2S)-2-methyl-1-(prop-2-enoyl)azetidin-2-yl]ethynyl}pyridin-4-yl)-1H,5H,6H,7H-pyrrolo[3,2-c]pyridin-4-one (18.10 mg, 47.89%) as a yellow solid.

LC-MS: (M+H)⁺ found 516.1.

¹H NMR (300 MHz, DMSO-d6) δ 11.14 (s, 1H), 8.76 (s, 1H), 8.40 (d, J=5.2 Hz, 1H), 8.34 (s, 1H), 8.01 (s, 1H), 7.16 (d, J=6.3 Hz, 2H), 6.88 (d, J=8.3 Hz, 1H), 6.77 (d, J=8.3 Hz, 1H), 6.20-5.94 (m, 2H), 5.53 (d, J=3.1 Hz, 1H), 3.73 (s, 3H), 3.36 (s, 2H), 3.11 (s, 2H), 2.83-2.66 (m, 2H), 2.08 (s, 2H), 1.69 (s, 3H).

### Example 185. 3-[(3-chloro-2-methoxyphenyl)amino]-2-{3-[2-(oxetan-3-yl)ethynyl]pyridin-4-yl}-1H,5H,6H,7H-pyrrolo[3,2-c]pyridin-4-one (compound 555)

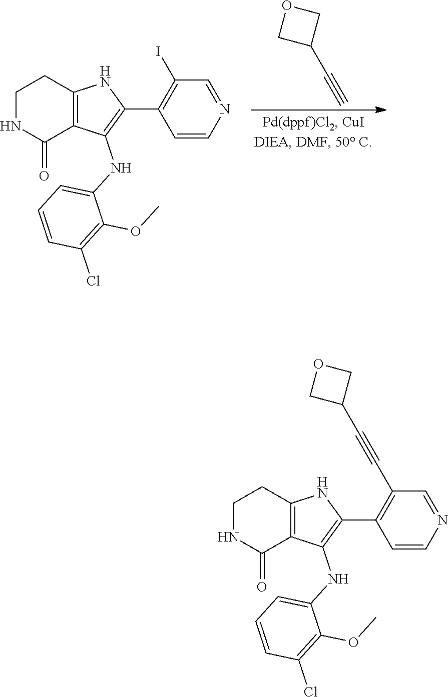

A solution of 3-[(3-chloro-2-methoxyphenyl)amino]-2-(3-iodopyridin-4-yl)-1H,5H,6H,7H-pyrrolo[3,2-c]pyridin-4-one (100.00 mg, 0.20 mmol, 1.00 equiv.) and 3-ethynyloxetane (83.00 mg, 1.01 mmol, 5.00 equiv.) and Pd(dppf)Cl₂·CH₂Cl₂ (16.00 mg, 0.02 mmol, 0.10 equiv.) and CuI (8.00 mg, 0.04 mmol, 0.20 equiv.) and TEA (61.00 mg, 0.61 mmol, 3.00 equiv.) in DMF (1 mL) was stirred for 2 h at 50° C. under nitrogen atmosphere. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 18% B to 38% B in 8 min, 38% B; Wave Length: 254/220 nm; RT1(min): 7.6; Number Of Runs: 0) to afford 3-[(3-chloro-2-methoxyphenyl)amino]-2-{3-[2-(oxetan-3-yl)ethynyl]pyridin-4-yl}-1H,5H,6H,7H-pyrrolo[3,2-c]pyridin-4-one (16.10 mg, 17.62%) as a dark yellow solid.

LC-MS: (M+H)⁺ found 449.0.

¹H NMR (400 MHz, DMSO-d6) δ 11.58 (s, 1H), 8.59 (s, 1H), 8.38 (d, J=5.3 Hz, 1H), 7.40 (s, 1H), 7.33 (d, J=5.3 Hz, 1H), 7.13 (t, J=2.5 Hz, 1H), 6.68-6.58 (m, 2H), 6.15-6.06 (m, 1H), 4.81-4.77 (m, 2H), 4.64-4.61 (m, 2H), 4.18-4.11 (m, 1H), 3.83 (s, 3H), 3.46-3.41 (m, 2H), 2.85 (t, J=6.8 Hz, 2H).

### Example 186. 3-[(3-fluoro-2-methoxyphenyl)amino]-2-[3-[2-(3-methyloxetan-3-yl)ethynyl]pyridin-4-yl]-1H,5H,6H,7H-pyrrolo[3,2-c]pyridin-4-one (compound 554)

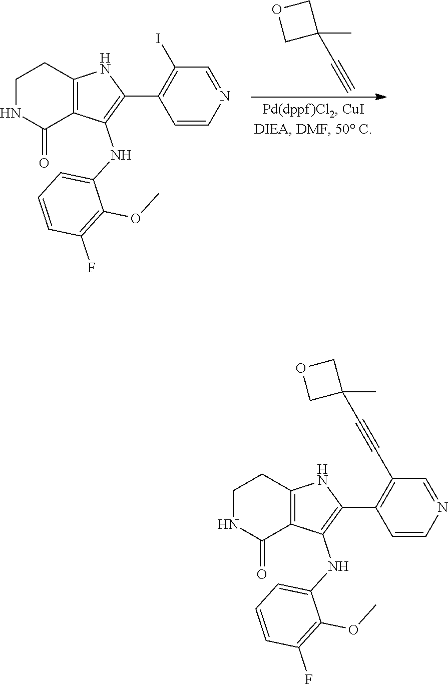

Into a 25 mL Schlenk tube were added 3-[(3-fluoro-2-methoxyphenyl)amino]-2-(3-iodopyridin-4-yl)-1H,5H,6H,7H-pyrrolo[3,2-c]pyridin-4-one (100.00 mg, 0.21 mmol, 1.00 equiv.) and Pd(dppf)Cl₂·CH₂Cl₂ (85.00 mg, 0.11 mmol, 0.50 equiv.) and CuI (20.00 mg, 0.11 mmol, 0.50 equiv.) and DIEA (81.00 mg, 0.63 mmol, 3.00 equiv.) and DMF (2 mL) at room temperature. To the above mixture was added 3-ethynyl-3-methyloxetane (100.50 mg, 1.05 mmol, 5.00 equiv.) dropwise at room temperature. The resulting mixture was stirred for 2 h at 50° C. under Argon atmosphere. The reaction was monitored by LCMS. The mixture was purified by silica gel column chromatography, eluted with CH₂Cl₂/MeOH (9:1) to afford crude product. The crude product (150.00 mg) was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 17% B to 37% B in 8 min, 37% B; Wave Length: 254/220 nm; RT1(min): 8; Number Of Runs: 0) to afford 3-[(3-fluoro-2-methoxyphenyl)amino]-2-[3-[2-(3-methyloxetan-3-yl)ethynyl]pyridin-4-yl]-1H,5H,6H,7H-pyrrolo[3,2-c]pyridin-4-one (26.50 mg, 28.33%) as a yellow solid.

LC-MS: (M+H)⁺ found 447.0.

¹H NMR (400 MHz, DMSO-d6) δ 11.50 (s, 1H), 8.57 (s, 1H), 8.38 (d, J=5.3 Hz, 1H), 7.42 (s, 1H), 7.33 (d, J=5.3 Hz, 1H), 7.13 (t, J=2.6 Hz, 1H), 6.57 (td, J=8.3, 6.0 Hz, 1H), 6.43 (ddd, J=11.0, 8.4, 1.5 Hz, 1H), 5.93 (dt, J=8.2, 1.3 Hz, 1H), 4.75 (d, J=5.4 Hz, 2H), 4.42 (d, J=5.4 Hz, 2H), 3.91-3.82 (m, 3H), 3.43 (td, J=6.9, 2.5 Hz, 2H), 2.84 (t, J=6.8 Hz, 2H), 1.63 (s, 3H)

### Example 187. 3-[(3-fluoro-2-methoxyphenyl)amino]-2-[3-[2-(3-methoxyoxetan-3-yl)ethynyl]pyridin-4-yl]-1H,5H,6H,7H-pyrrolo[3,2-c]pyridin-4-one (compound 553)

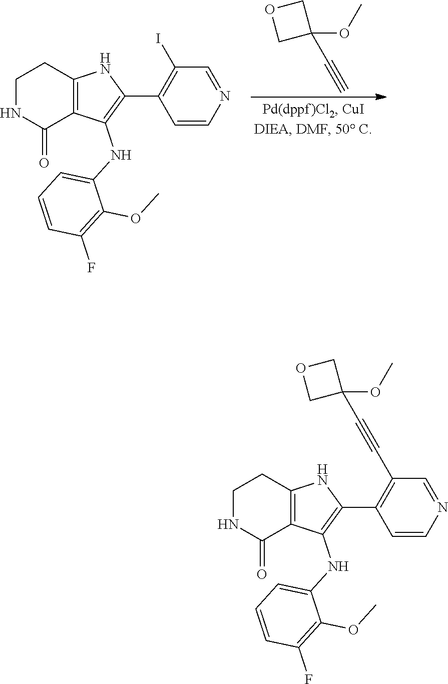

Into a 25 mL Schlenk tube were added 3-[(3-fluoro-2-methoxyphenyl)amino]-2-(3-iodopyridin-4-yl)-1H,5H,6H,7H-pyrrolo[3,2-c]pyridin-4-one (100.00 mg, 0.21 mmol, 1.00 equiv.) and Pd(dppf)Cl₂·CH₂Cl₂ (85.00 mg, 0.11 mmol, 0.50 equiv.) and CuI (20.00 mg, 0.11 mmol, 0.50 equiv.) and DIEA (81.00 mg, 0.63 mmol, 3.00 equiv.) and DMF (2 mL) at room temperature. To the above mixture was added 3-ethynyl-3-methoxyoxetane (117.22 mg, 1.05 mmol, 5.00 equiv.) dropwise at room temperature. The resulting mixture was stirred for 2 h at 50° C. under Argon atmosphere. The reaction was monitored by LCMS. The mixture was purified by silica gel column chromatography, eluted with CH₂Cl₂/MeOH (9:1) to afford crude product. The crude product (150.00 mg) was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 20% B to 41% B in 8 min, 41% B; Wave Length: 254/220 nm; RT1(min): 7.33; Number Of Runs: 0) to afford 3-[(3-fluoro-2-methoxyphenyl)amino]-2-[3-[2-(3-methoxyoxetan-3-yl)ethynyl]pyridin-4-yl]-1H,5H,6H,7H-pyrrolo[3,2-c]pyridin-4-one (46.30 mg, 47.59%) as a yellow solid.

LC-MS: (M+H)⁺ found 463.0.

¹H NMR (400 MHz, DMSO-d6) δ11.59 (s, 1H), 8.68 (s, 1H), 8.44 (d, J=5.2 Hz, 1H), 7.40 (s, 1H), 7.37 (d, J=5.3 Hz, 1H), 7.14 (d, J=2.6 Hz, 1H), 6.57 (td, J=8.3, 6.1 Hz, 1H), 6.42 (ddd, J=11.0, 8.4, 1.5 Hz, 1H), 5.94 (dt, J=8.3, 1.2 Hz, 1H), 4.76 (d, J=6.9 Hz, 2H), 4.64 (d, J=6.8 Hz, 2H), 3.86 (s, 3H), 3.42 (td, J=6.9, 2.5 Hz, 2H), 3.27 (s, 3H), 2.82 (t, J=6.8 Hz, 2H).

### Example 188. 3-[(3-fluoro-2-methoxyphenyl)amino]-2-[3-[2-(oxetan-3-yl)ethynyl]pyridin-4-yl]-1H,5H,6H,7H-pyrrolo[3,2-c]pyridin-4-one (compound 552)

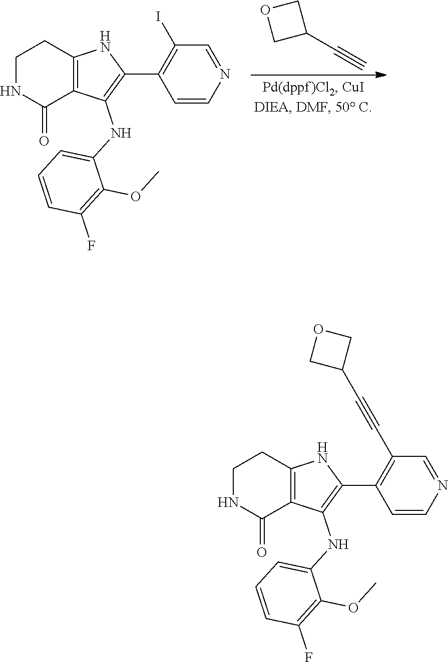

Into a 25 mL Schlenk tube were added 3-[(3-fluoro-2-methoxyphenyl)amino]-2-(3-iodopyridin-4-yl)-1H,5H,6H,7H-pyrrolo[3,2-c]pyridin-4-one (100.00 mg, 0.21 mmol, 1.00 equiv.) and Pd(dppf)Cl₂·CH₂Cl₂ (85.00 mg, 0.11 mmol, 0.50 equiv.) and CuI (20.00 mg, 0.11 mmol, 0.50 equiv.) and DIEA (81.00 mg, 0.63 mmol, 3.00 equiv.) and DMF (2 mL) at room temperature. To the above mixture was added 3-ethynyloxetane (86.00 mg, 1.05 mmol, 5.00 equiv.) dropwise at room temperature. The resulting mixture was stirred for 2 h at 50° C. under Argon atmosphere. The reaction was monitored by LCMS. The mixture was purified by silica gel column chromatography, eluted with CH₂Cl₂/MeOH (9:1) to afford crude product. The crude product (150.00 mg) was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 17% B to 37% B in 8 min, 37% B; Wave Length: 254/220 nm; RT1(min): 8; Number Of Runs: 0) to afford 3-[(3-fluoro-2-methoxyphenyl)amino]-2-[3-[2-(oxetan-3-yl)ethynyl]pyridin-4-yl]-1H,5H,6H,7H-pyrrolo[3,2-c]pyridin-4-one (49.60 mg, 54.58%) as a yellow solid.

LC-MS: (M+H)⁺ found 432.0.

¹H NMR (400 MHz, DMSO-d6) δ 11.53 (s, 1H), 8.52 (d, J=84.7 Hz, 2H), 7.45 (s, 1H), 7.36 (d, J=4.3 Hz, 1H), 7.15 (d, J=2.5 Hz, 1H), 6.57 (td, J=8.3, 6.1 Hz, 1H), 6.44 (ddd, J=10.9, 8.3, 1.5 Hz, 1H), 5.95 (dt, J=8.3, 1.3 Hz, 1H), 4.79 (dd, J=8.5, 5.4 Hz, 2H), 4.63 (dd, J=7.1, 5.4 Hz, 2H), 4.16 (tt, J=8.6, 7.1 Hz, 1H), 3.87 (s, 3H), 3.43 (td, J=6.9, 2.5 Hz, 2H), 2.85 (t, J=6.8 Hz, 2H).

### Example 189. 3-[(3-chloro-2-methylphenyl)amino]-2-(3-{2-[1-(difluoromethyl)cyclopropyl]ethynyl}pyridin-4-yl)-1H,5H,6H,7H-pyrrolo[3,2-c]pyridin-4-one (compound 556)

### 189.1. Synthesis of 1-chloro-3-isothiocyanato-2-methylbenzene

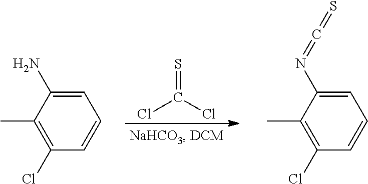

A solution of 2-methyl-3-chloroaniline (20.00 g, 141.24 mmol, 1.00 equiv.) in DCM (100 mL) and NaHCO₃(aq. 100 mL) was added thiophosgene (16.24 g, 141.24 mmol, 1.00 equiv.) dropwise at 0° C. under nitrogen atmosphere. The mixture was stirred for 2 h at room temperature under nitrogen atmosphere. The reaction was monitored by TLC. The resulting mixture was extracted with DCM (3×100 mL). The combined organic layers were washed with brine, dried over anhydrous Na₂SO₄. After filtration, the filtrate was concentrated under reduced pressure. The residue was purified by silica gel column chromatography, eluted with PE to afford 1-chloro-3-isothiocyanato-2-methylbenzene (21.00 g, 80.96%) as a yellow oil.

### 189.2. Synthesis of tert-butyl 3-[(3-chloro-2-methylphenyl)carbamothioyl]-4-hydroxy-2-oxo-5,6-dihydropyridine-1-carboxylate

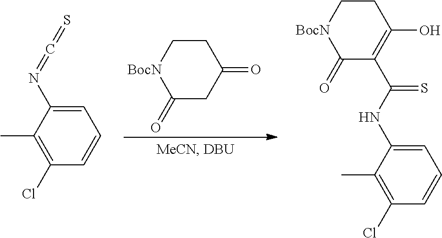 [SMILES: Cc1c(Cl)cccc1N=C=S ; CC(C)(C)OC(=O)N1CCC(=O)CC1=O ; Cc1c(Cl)cccc1NC(=S)C1=C(O)CCN(C(=O)OC(C)(C)C)C1=O]

A solution of tert-butyl 2,4-dioxopiperidine-1-carboxylate (21.40 g, 100.36 mmol, 1.00 equiv.) and 1-chloro-3-isothiocyanato-2-methylbenzene (20.27 g, 110.40 mmol, 1.10 equiv.) in ACN (200 mL) was added DBU (22.92 g, 150.54 mmol, 1.50 equiv.) and then was stirred at room temperature for 2 h. The mixture was acidified to pH 6 with HCl (aq.). The precipitated solids were collected by filtration and washed with water (3×10 mL) to afford tert-butyl 3-[(3-chloro-2-methylphenyl)carbamothioyl]-4-hydroxy-2-oxo-5,6-dihydropyridine-1-carboxylate (32.20 g, 80.84%) as a yellow solid.

LC-MS: (M+H)⁺ found 397.0.

### 189.3. Synthesis of tert-butyl 4-[[(3-bromopyridin-4-yl)methyl]amino]-3-[(3-chloro-2-methylphenyl)carbamothioyl]-2-oxo-5,6-dihydropyridine-1-carboxylate

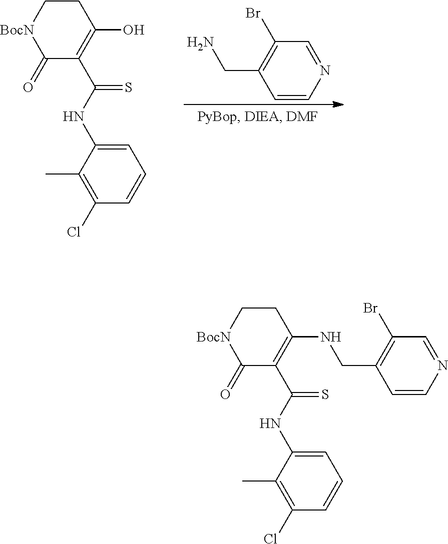 [SMILES: Cc1c(Cl)cccc1NC(=S)C1=C(O)CCN(C(=O)OC(C)(C)C)C1=O ; NCc1ccncc1Br ; Cc1c(Cl)cccc1NC(=S)C1=C(NCc2ccncc2Br)CCN(C(=O)OC(C)(C)C)C1=O]

To a stirred mixture of tert-butyl 3-[(3-chloro-2-methylphenyl)carbamothioyl]-4-hydroxy-2-oxo-5,6-dihydropyridine-1-carboxylate (21.00 g, 52.91 mmol, 1.00 equiv.) and 1-(3-bromopyridin-4-yl)methanamine (9.90 g, 52.91 mmol, 1.00 equiv.) in DMF (400 mL) were added PyBop (41.30 g, 79.37 mmol, 1.50 equiv.) and DIEA (13.68 g, 105.82 mmol, 2.00 equiv.) at 0° C. under nitrogen atmosphere. The resulting mixture was stirred for 2 h at room temperature under nitrogen atmosphere. The reaction was quenched with Water. The resulting mixture was extracted with EA (3×300 mL). The combined organic layers were washed with brine (3×200 mL), dried over anhydrous Na₂SO₄. After filtration, the filtrate was concentrated under reduced pressure. The residue was purified by silica gel column chromatography, eluted with EA in PE (0% to 100% gradient in 40 min) to afford tert-butyl 4-[[(3-bromopyridin-4-yl)methyl]amino]-3-[(3-chloro-2-methylphenyl)carbamothioyl]-2-oxo-5,6-dihydropyridine-1-carboxylate (26.20 g, 87.50%) as a yellow solid.

LC-MS: (M+H)⁺ found 565.0.

### 189.4. Synthesis of 2-(3-bromopyridin-4-yl)-3-[(3-chloro-2-methylphenyl)amino]-1H,5H,6H,7H-pyrrolo[3,2-c]pyridin-4-one

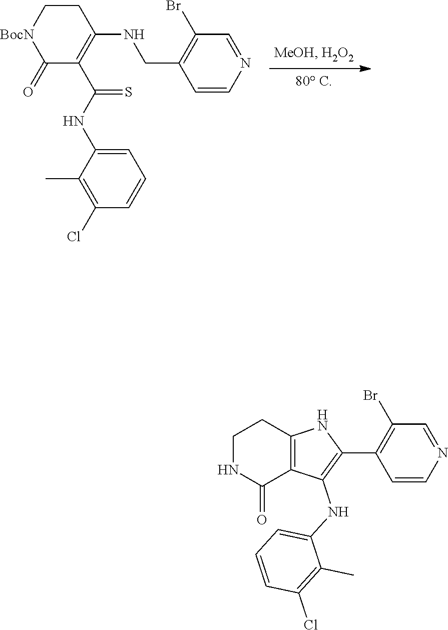 [SMILES: Cc1c(Cl)cccc1NC(=S)C1=C(NCc2ccncc2Br)CCN(C(=O)OC(C)(C)C)C1=O ; Cc1c(Cl)cccc1Nc1c(-c2ccncc2Br)[nH]c2c1C(=O)NCC2]

To a stirred mixture of tert-butyl 4-[[(3-bromopyridin-4-yl)methyl]amino]-3-[(3-chloro-2-methylphenyl)carbamothioyl]-2-oxo-5,6-dihydropyridine-1-carboxylate (26.00 g, 45.94 mmol, 1.00 equiv.) in MeOH (300 mL) was added H₂O₂ (6.77 g, 59.73 mmol, 1.30 equiv., 30w/w %) at room temperature. The resulting mixture was stirred for overnight at 80° C. The reaction was quenched by the addition of sat. Na₂SO₃ (aq.). The resulting mixture was concentrated under reduced pressure. The residue was purified by reverse flash chromatography with the following conditions (column: C18 silica gel; mobile phase, ACN in water, 0% to 60% gradient in 30 min; detector, UV 254 nm.) to afford 2-(3-bromopyridin-4-yl)-3-[(3-chloro-2-methylphenyl)amino]-1H,5H,6H,7H-pyrrolo[3,2-c]pyridin-4-one (8.50 g, 42.85%) as a yellow solid.

LC-MS: (M+H)⁺ found 431.0.

### 189.5. Synthesis of 3-[(3-chloro-2-methylphenyl)amino]-2-(3-{2-[1-(difluoromethyl)cyclopropyl]ethynyl}pyridin-4-yl)-1H,5H,6H,7H-pyrrolo[3,2-c]pyridin-4-one

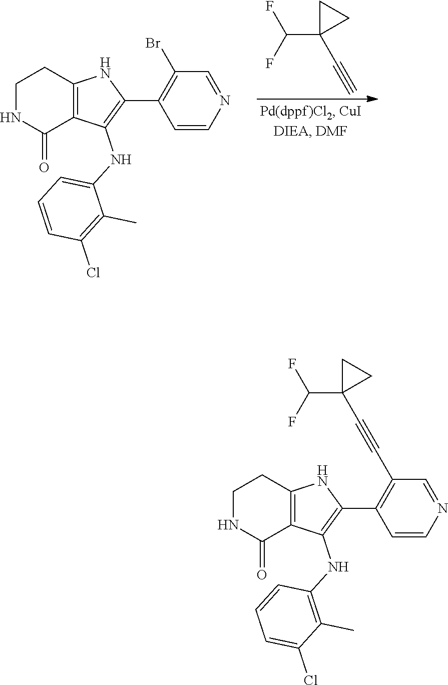

To a stirred solution of 2-(3-bromopyridin-4-yl)-3-[(3-chloro-2-methylphenyl)amino]-1H,5H,6H,7H-pyrrolo[3,2-c]pyridin-4-one (100.00 mg, 0.23 mmol, 1.00 equiv.) and 1-(difluoromethyl)-1-ethynylcyclopropane (54.00 mg, 0.46 mmol, 2.00 equiv.) in DMF (2.5 mL) was added Pd(dppf)Cl₂·CH₂Cl₂ (94.00 mg, 0.12 mmol, 0.50 equiv.) and CuI (22.00 mg, 0.12 mmol, 0.50 equiv.) and DIEA (90.00 mg, 0.69 mmol, 3.00 equiv.), then stirred overnight at 50° C. under Argon atmosphere. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 40% B to 52% B in 10 min, 52% B; Wave Length: 220/254 nm; RT1(min): 9.25; Number Of Runs: 0) to afford 3-[(3-chloro-2-methylphenyl)amino]-2-(3-{2-[1-(difluoromethyl)cyclopropyl]ethynyl}pyridin-4-yl)-1H,5H,6H,7H-pyrrolo[3,2-c]pyridin-4-one (27.70 mg, 25.36%) as a yellow solid.

LC-MS: (M+H)⁺ found 417.0.

¹H NMR (400 MHz, DMSO-d6) δ 11.34 (s, 1H), 8.54 (s, 1H), 8.32 (s, 1H), 7.31-7.10 (m, 3H), 6.74-6.66 (m, 2H), 6.17 (d, J=7.8 Hz, 1H), 5.81 (t, J=55.7 Hz, 1H), 3.46-3.41 (m, 2H), 2.83 (t, J=6.8 Hz, 2H), 2.30 (s, 3H), 1.28-1.19 (m, 4H).

### Example 190. 3-((3-fluoro-2-methoxyphenyl)amino)-2-(3-((1-(trifluoromethyl)cyclopropyl)ethynyl)pyridin-4-yl)-1,5,6,7-tetrahydro-4H-pyrrolo[3,2-c]pyridin-4-one (compound 559)

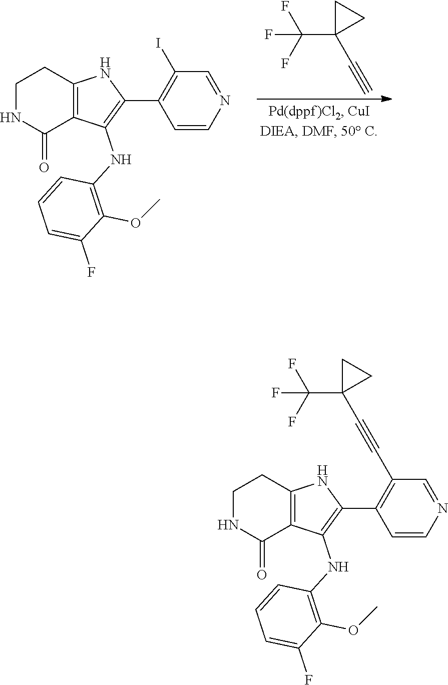

Into a 25 mL Schlenk tube were added 3-[(3-fluoro-2-methoxyphenyl)amino]-2-(3-iodopyridin-4-yl)-1H,5H,6H,7H-pyrrolo[3,2-c]pyridin-4-one (100.00 mg, 0.21 mmol, 1.00 equiv.) and Pd(dppf)Cl₂·CH₂Cl₂ (85.00 mg, 0.11 mmol, 0.50 equiv.) and CuI (20.00 mg, 0.11 mmol, 0.50 equiv.) and DIEA (81.00 mg, 0.63 mmol, 3.00 equiv.) and DMF (2 mL) at room temperature. To the above mixture was added 1-ethynyl-1-(trifluoromethyl)cyclopropane (56.00 mg, 0.42 mmol, 2.00 equiv.) dropwise at room temperature. The resulting mixture was stirred for 2 h at 50° C. under Argon atmosphere. The reaction was monitored by LCMS. The mixture was purified by silica gel column chromatography, eluted with CH₂Cl₂/MeOH (9:1) to afford crude product. The crude product (80.00 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 34% B to 64% B in 7 min; Wave Length: 254 nm; RT1(min): 6.5; Number Of Runs: 0) to afford 3-((3-fluoro-2-methoxyphenyl)amino)-2-(3-((1-(trifluoromethyl)cyclopropyl)ethynyl)pyridin-4-yl)-1,5,6,7-tetrahydro-4H-pyrrolo[3,2-c]pyridin-4-one (49.60 mg, 54.58%) as a light yellow solid.

LC-MS: (M+H)⁺ found 485.0.

¹H NMR (300 MHz, DMSO-d6) δ 11.45 (s, 1H), 8.58 (s, 1H), 8.40 (d, J=5.3 Hz, 1H), 7.36 (s, 1H), 7.33 (d, J=5.3 Hz, 1H), 7.12 (d, J=2.6 Hz, 1H), 6.56 (td, J=8.3, 6.0 Hz, 1H), 6.42 (ddd, J=10.1, 8.4, 1.5 Hz, 1H), 5.91 (d, J=8.3 Hz, 1H), 3.87 (s, 3H), 3.43 (td, J=6.9, 2.5 Hz, 2H), 2.82 (t, J=6.8 Hz, 2H), 1.47 (td, J=6.4, 2.5 Hz, 2H), 1.43-1.28 (m, 2H).

### Example 191. 3-[(3-chloro-2-methoxyphenyl)amino]-2-(3-{2-[1-(fluoromethyl)cyclopropyl]ethynyl}pyridin-4-yl)-1H,5H,6H,7H-pyrrolo[3,2-c]pyridin-4-one (compound 561)

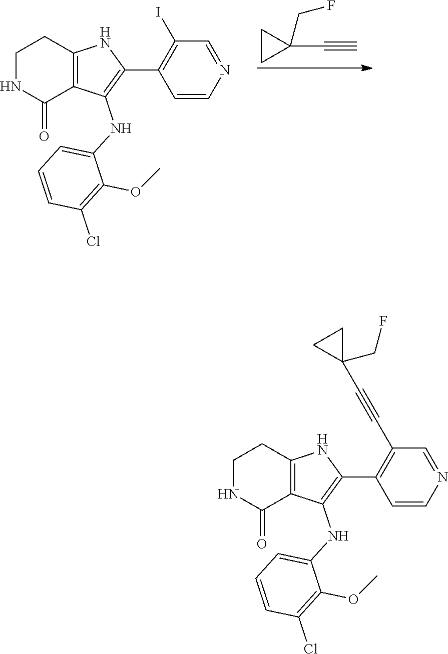

Into a 20 mL sealed tube were added 3-[(3-chloro-2-methoxyphenyl)amino]-2-(3-iodopyridin-4-yl)-1H,5H,6H,7H-pyrrolo[3,2-c]pyridin-4-one (100.00 mg, 0.20 mmol, 1.00 equiv.), CuI (19 mg, 0.10 mmol, 0.5 equiv.), Pd(dppf)Cl₂·CH₂Cl₂ (82 mg, 0.10 mmol, 0.50 equiv.), DMF (2 mL), 1-ethynyl-1-(fluoromethyl) cyclopropane (99.00 mg, 1.01 mmol, 5.00 equiv.) and DIEA (0.50 mL, 2.86 mmol, 14.3equiv.) at room temperature. The resulting mixture was stirred for 2 h at 50° C. under Argon atmosphere. The reaction was monitored by LCMS. The resulting mixture was filtered, the filter cake was washed with CH₂Cl₂ (3×10 mL). The filtrate was concentrated under reduced pressure. The residue was purified by silica gel column chromatography, eluted with CH₂Cl₂/MeOH (10:1) to afford 3-[(3-chloro-2-methoxyphenyl)amino]-2-(3-{2-[1-(fluoromethyl)cyclopropyl]ethynyl}pyridin-4-yl)-1H,5H,6H,7H-pyrrolo[3,2-c]pyridin-4-one (8.10 mg, 8.62%) as a yellow solid.

LC-MS: (M+H)⁺ found 465.0.

¹H NMR (300 MHz, Methanol-d4) δ 8.50 (s, 1H), 8.21 (s, 1H), 7.66-7.47 (m, 1H), 7.39 (d, J=5.3 Hz, 1H), 6.69-6.56 (m, 1H), 6.21 (dd, J=7.6, 2.0 Hz, 1H), 4.49 (s, 1H), 4.33 (s, 1H), 3.95 (s, 3H), 3.61 (t, J=6.9 Hz, 2H), 2.97 (t, J=6.9 Hz, 2H), 1.27-1.18 (m, 2H), 1.09 (t, J=3.5 Hz, 2H).

### Example 192. 2-[3-(2-cyclopropylethynyl)pyridin-4-yl]-3-[(3-fluoro-2-methoxyphenyl)amino]-1H,5H,6H,7H-pyrrolo[3,2-c]pyridin-4-one (compound 563)

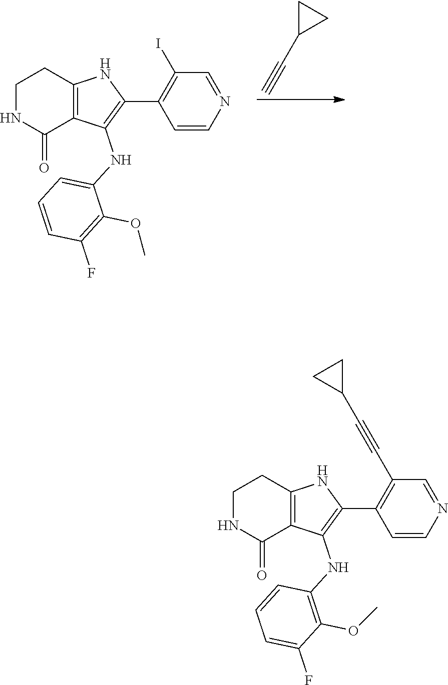

A solution of 3-[(3-fluoro-2-methoxyphenyl)amino]-2-(3-iodopyridin-4-yl)-1H,5H,6H,7H-pyrrolo[3,2-c]pyridin-4-one (100.00 mg, 0.21 mmol, 1.00 equiv.) and ethynylcyclopropane (69.00 mg, 1.05 mmol, 5.00 equiv.) and Pd(dppf)Cl₂·CH₂Cl₂ (17.00 mg, 0.02 mmol, 0.10 equiv.) and CuI (8.00 mg, 0.04 mmol, 0.20 equiv.) and TEA (63.00 mg, 0.63 mmol, 3.00 equiv.) in DMF (1 mL) was stirred for 2 h at 50° C. under nitrogen atmosphere. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 26% B to 50% B in 9 min, 50% B; Wave Length: 254/220 nm; RT1(min): 8.85; Number Of Runs: 0) to afford 2-[3-(2-cyclopropylethynyl)pyridin-4-yl]-3-[(3-fluoro-2-methoxyphenyl)amino]-1H,5H,6H,7H-pyrrolo[3,2-c]pyridin-4-one (19.10 mg, 21.85%) as a dark yellow solid.

LC-MS: (M+H)⁺ found 417.0.

¹H NMR (400 MHz, DMSO-d6) δ 11.43 (s, 1H), 8.53 (s, 1H), 8.33 (d, J=5.1 Hz, 1H), 7.42-7.22 (m, 2H), 7.12 (s, 1H), 6.58 (q, J=7.4 Hz, 1H), 6.42 (t, J=9.7 Hz, 1H), 5.95 (d, J=8.3 Hz, 1H), 3.88 (s, 3H), 3.44 (t, J=6.8 Hz, 2H), 2.85 (t, J=6.8 Hz, 2H), 1.61-1.54 (m, 1H), 0.92-0.79 (m, 4H).

### Example 193. 3-[(3-chloro-2-methylphenyl)amino]-2-[3-(2-cyclopropylethynyl)pyridin-4-yl]-1H,5H,6H,7H-pyrrolo[3,2-c]pyridin-4-one (compound 562)

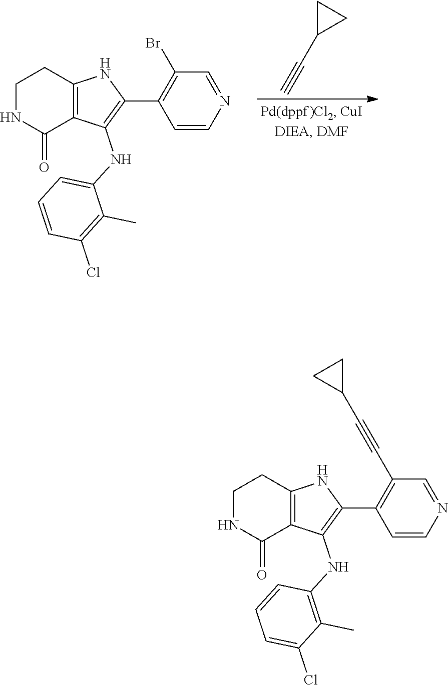

Into a 25 mL Schlenk tube were added 2-(3-bromopyridin-4-yl)-3-[(3-chloro-2-methylphenyl)amino]-1H,5H,6H,7H-pyrrolo[3,2-c]pyridin-4-one (100.00 mg, 0.23 mmol, 1.00 equiv.) and Pd(dppf)Cl₂·CH₂Cl₂ (94.00 mg, 0.12 mmol, 0.50 equiv.) and CuI (44.00 mg, 0.23 mmol, 1.00 equiv.) and DIEA (180.00 mg, 1.39 mmol, 6.00 equiv.) and DMF (2 mL) at room temperature. To the above mixture was added ethynylcyclopropane (46.00 mg, 0.70 mmol, 3.00 equiv.) dropwise at room temperature. The resulting mixture was stirred for 2 h at 65° C. under Argon atmosphere. The reaction was monitored by LCMS. The mixture was purified by silica gel column chromatography, eluted with CH₂Cl₂/MeOH (9:1) to afford crude product. The crude product (80.00 mg) was purified by Prep-HPLC with the following conditions (Column: XBridge Prep OBD C18 Column, 30*150 mm, 5 μm; Mobile Phase A: Water (0.05% FA), Mobile Phase B: ACN; Flow rate: 60 mL/min; Gradient: 22% B to 52% B in 7 min; Wave Length: 254 nm; RT1(min): 6.5; Number Of Runs: 0) to afford 3-[(3-chloro-2-methylphenyl)amino]-2-[3-(2-cyclopropylethynyl)pyridin-4-yl]-1H,5H,6H,7H-pyrrolo[3,2-c]pyridin-4-one (5.20 mg, 5.12%) as a light yellow solid.

LC-MS: (M+H)⁺ found 417.0.

¹H NMR (300 MHz, DMSO-d6) δ 11.37 (s, 1H), 8.49 (s, 1H), 8.28 (d, J=5.3 Hz, 1H), 7.23 (t, J=4.5 Hz, 2H), 7.14 (s, 1H), 6.69 (d, J=7.6 Hz, 2H), 6.17 (d, J=7.2 Hz, 1H), 3.44 (s, 2H), 2.85 (s, 2H), 2.32 (s, 3H), 1.57 (s, 1H), 0.91 (dt, J=6.0, 3.0 Hz, 2H), 0.77 (dd, J=5.1, 2.7 Hz, 2H).

### Example 194. 3-[(3-chloro-2-methoxyphenyl)amino]-2-[3-[2-(1-hydroxycyclopropyl)ethynyl] pyridin-4-yl]-1H,5H,6H,7H-pyrrolo[3,2-c]pyridin-4-one (compound 564)

### 194.1. Synthesis of 1-ethoxycyclopropan-1-ol

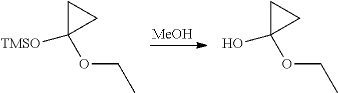

Into a 50 mL round-bottom flask was added (1-ethoxycyclopropoxy)trimethylsilane (2.00 g, 11.47 mmol, 1.00 equiv.) in methanol (10.00 mL). The resulting mixture was stirred for 8 h at room temperature under nitrogen atmosphere. The resulting mixture was concentrated under reduced pressure to give 1-ethoxycyclopropan-1-ol (1.00 g, 85.34%) as light yellow oil.

¹H NMR (400 MHz, Chloroform-d) δ 3.77 (q, J=7.1 Hz, 2H), 3.52 (brs, 1H), 1.22 (t, J=7.1 Hz, 3H), 0.94 (dt, J=8.0, 2.1 Hz, 4H).

### 194.2. Synthesis of 1-[2-(trimethylsilyl)ethynyl]cyclopropan-1-ol

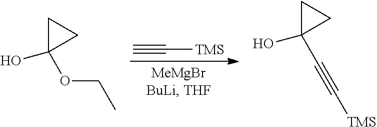

To a stirred solution of 1-ethoxycyclopropan-1-ol (1.00 g, 9.79 mmol, 1.00 equiv.) in THF (7.50 mL) was added MeMgBr (4.24 mL, 12.73 mmol, 1.30 equiv.) dropwise at 0° C. under nitrogen atmosphere. The reaction was stirred for 1 h. To a stirred solution of trimethylsilylacetylene (1.06 g, 10.79 mmol, 1.10 equiv.) in THF (7.50 mL) was added n-BuLi (4.50 mL, 11.26 mmol, 1.15 equiv.) dropwise at −78° C. under nitrogen atmosphere. The resulting [(trimethylsilyl)ethynyl] lithium was added into the solution of bromide magnesium 1-ethoxyl cyclopropanolate at 0° C. The mixture was allowed to warm to room temperature for 16 h. The reaction was quenched with sat. NH₄C1 (aq.) at 0° C. The resulting mixture was extracted with EtOAc (3×20 mL). The combined organic layers were washed with brine (2×20 mL), dried over anhydrous Na₂SO₄. After filtration, the filtrate was concentrated under reduced pressure to give 1-[2-(trimethylsilyl)ethynyl]cyclopropan-1-ol (600.00 mg, 39.72%) as a yellow oil. GC-MS: (M+H)⁺ found 154.0.

### 194.3. Synthesis of 3-[(3-chloro-2-methoxyphenyl)amino]-2-[3-[2-(1-hydroxycyclopropyl)ethynyl] pyridin-4-yl]-1H,5H,6H,7H-pyrrolo[3,2-c]pyridin-4-one

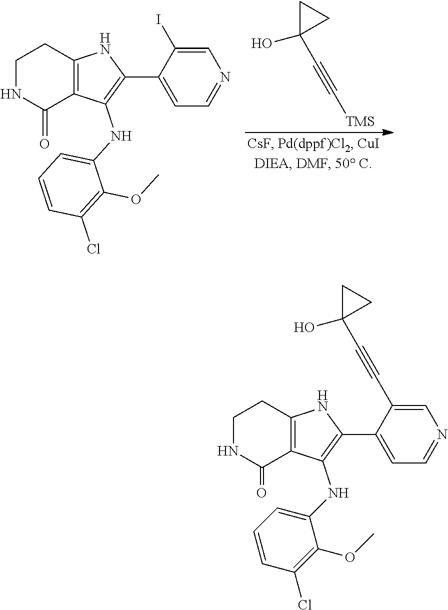 [SMILES: COc1c(Cl)cccc1Nc1c(-c2ccncc2I)[nH]c2c1C(=O)NCC2 ; C[Si](C)(C)C#CC1(O)CC1 ; "CsF, Pd(dppf)Cl2, CuIDIEA, DMF, 50° C." ; COc1c(Cl)cccc1Nc1c(-c2ccncc2C#CC2(O)CC2)[nH]c2c1C(=O)NCC2]

Into a 25 mL Schlenk tube were added 3-[(3-chloro-2-methoxyphenyl)amino]-2-(3-iodopyridin-4-yl)-1H,5H,6H,7H-pyrrolo[3,2-c]pyridin-4-one (200.00 mg, 0.40 mmol, 1.00 equiv.) and Pd(dppf)C12 (147.90 mg, 0.20 mmol, 0.50 equiv.) and CuI (38.50 mg, 0.20 mmol, 0.50 equiv.) and DIEA (156.75 mg, 1.21 mmol, 3.00 equiv.) and DMF (4.00 mL) at room temperature. To the above mixture was added 1-[2-(trimethylsilyl)ethynyl]cyclopropan-1-ol (149.69 mg, 0.97 mmol, 2.40 equiv.) dropwise at room temperature. The resulting mixture was stirred for 2 h at 50° C. under Argon atmosphere. The reaction was monitored by LCMS. The mixture was purified by silica gel column chromatography, eluted with CH₂Cl₂/MeOH (9:1) to afford crude product. The crude product (350.00 mg) was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 19% B to 37% B in 7 min, 37% B; Wave Length: 254/220 nm; RT1(min): 7; Number Of Runs: 0) to afford 3-[(3-chloro-2-methoxyphenyl)amino]-2-[3-[2-(1-hydroxycyclopropyl)ethynyl] pyridin-4-yl]-1H,5H,6H,7H-pyrrolo[3,2-c]pyridin-4-one (14.20 mg, 7.79%) as a yellow solid.

LC-MS: (M+H)⁺ found 449.0.

¹H NMR (400 MHz, Methanol-d4) δ 8.54 (s, 1H), 8.23 (d, J=5.5 Hz, 1H), 7.42 (d, J=5.4 Hz, 1H), 6.65 (d, J=8.0 Hz, 1H), 6.60 (t, J=8.0 Hz, 1H), 6.20 (dd, J=7.9, 1.7 Hz, 1H), 3.96 (s, 3H), 3.61 (t, J=6.9 Hz, 2H), 2.98 (t, J=6.9 Hz, 2H), 1.15 (s, 4H).

### Example 195. 2-(3-{2-[(2R)-1-(but-2-ynoyl)pyrrolidin-2-yl]ethynyl}pyridin-4-yl)-3-[(3-chloro-2-methoxyphenyl)amino]-1H,5H,6H,7H-pyrrolo[3,2-c]pyridin-4-one (compound 208)

### 195.1. Synthesis of 3-[(3-chloro-2-methoxyphenyl)amino]-2-{3-[2-(pyrrolidin-2-yl)ethynyl]pyridin-4-yl}-1H,5H,6H,7H-pyrrolo[3,2-c]pyridin-4-one

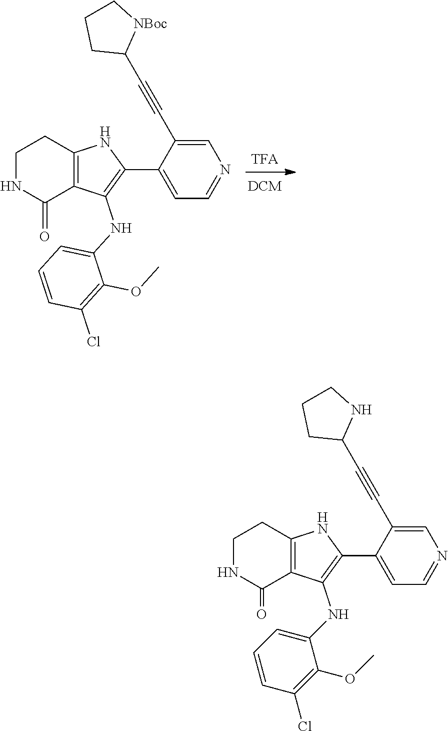 [SMILES: COc1c(Cl)cccc1Nc1c(-c2ccncc2C#CC2CCCN2C(=O)OC(C)(C)C)[nH]c2c1C(=O)NCC2 ; COc1c(Cl)cccc1Nc1c(-c2ccncc2C#CC2CCCN2)[nH]c2c1C(=O)NCC2]

To a stirred mixture of tert-butyl 2-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]pyrrolidine-1-carboxylate (100.00 mg, 0.18 mmol, 1.00 equiv.) in DCM (1.5 mL) was added TFA (0.5 mL) at room temperature under nitrogen atmosphere. The resulting mixture was stirred for 1 h and concentrated under reduced pressure to afford 3-[(3-chloro-2-methoxyphenyl)amino]-2-{3-[2-(pyrrolidin-2-yl)ethynyl]pyridin-4-yl}-1H,5H,6H,7H-pyrrolo[3,2-c]pyridin-4-one (220.00 mg, 91.01%) as a red oil. The crude product was used in the next step directly without further purification.

LC-MS: (M+H)⁺ found 462.1.

### 195.2. Synthesis of 2-(3-{2-[1-(but-2-ynoyl)pyrrolidin-2-yl]ethynyl}pyridin-4-yl)-3-[(3-chloro-2-methoxyphenyl)amino]-1H,5H,6H,7H-pyrrolo[3,2-c]pyridin-4-one

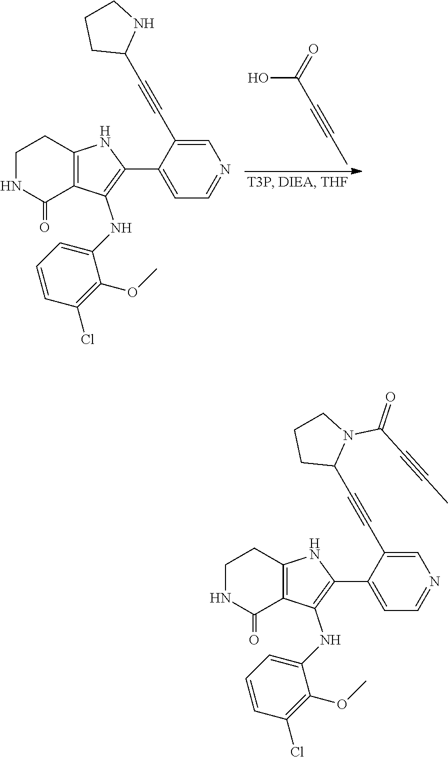

A solution of 3-[(3-chloro-2-methoxyphenyl)amino]-2-{3-[2-(pyrrolidin-2-yl)ethynyl]pyridin-4-yl}-1H,5H,6H,7H-pyrrolo[3,2-c]pyridin-4-one (200.00 mg, 0.15 mmol, 1.00 equiv.) in THF (5 mL) was basified to pH 8 with DIEA. To the above mixture was added 2-butynoic acid (25.00 mg, 0.29 mmol, 2.00 equiv.) at 0° C. under nitrogen atmosphere followed by the addition of T₃P (140.00 mg, 0.22 mmol, 1.50 equiv.) dropwise. The resulting mixture was stirred for 1 h at room temperature under nitrogen atmosphere. Saturated aqueous NaHCO₃(10 mL) was added to the reaction mixture at 0° C. and extracted with EtOAc (3×10 mL), dried over anhydrous Na₂SO₄ and filtered. The filtrate was concentrated under reduced pressure to give crude product (60 mg) that was purified by Prep-HPLC under the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 13% B to 43% B in 7 min, 43% B; Wave Length: 254 nm; RT1(min): 6.07; Number Of Runs: 0) to afford 2-(3-{2-[1-(but-2-ynoyl)pyrrolidin-2-yl]ethynyl}pyridin-4-yl)-3-[(3-chloro-2-methoxyphenyl)amino]-1H,5H,6H,7H-pyrrolo[3,2-c]pyridin-4-one (56.00 mg, 72.05%) as a yellow solid.

LC-MS: (M+H)⁺ found 528.2.

### 195.3. Synthesis of 2-(3-{2-[(2R)-1-(but-2-ynoyl)pyrrolidin-2-yl]ethynyl}pyridin-4-yl)-3-[(3-chloro-2-methoxyphenyl)amino]-1H,5H,6H,7H-pyrrolo[3,2-c]pyridin-4-one

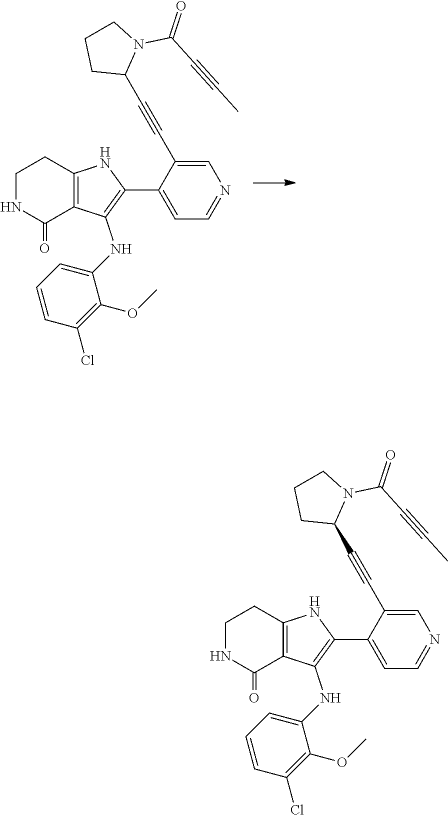 [SMILES: CC#CC(=O)N1CCCC1C#Cc1cnccc1-c1[nH]c2c(c1Nc1cccc(Cl)c1OC)C(=O)NCC2 ; CC#CC(=O)N1CCC[C@@H]1C#Cc1cnccc1-c1[nH]c2c(c1Nc1cccc(Cl)c1OC)C(=O)NCC2]

2-(3-{2-[1-(but-2-ynoyl)pyrrolidin-2-yl]ethynyl}pyridin-4-yl)-3-[(3-chloro-2-methoxyphenyl)amino]-1H,5H,6H,7H-pyrrolo[3,2-c]pyridin-4-one (56.00 mg, 0.11 mmol, 1.00 equiv.) was purified by Prep-HPLC with the following conditions (Column: CHIRAL ART Amylose-SA, 2*25 cm, 5 μm; Mobile Phase A: MTBE (0.5% 2M NH₃-MeOH)—HPLC, Mobile Phase B: IPA—HPLC; Flow rate: 20 mL/min; Gradient: 30% B to 30% B in 15 min; Wave Length: 220/254 nm; RT1(min): 9.646; RT2(min): 13.616; Sample Solvent: EtOH—HPLC; Injection Volume: 0.67 mL; Number Of Runs: 6) to afford 2-(3-{2-[(2R)-1-(but-2-ynoyl)pyrrolidin-2-yl]ethynyl}pyridin-4-yl)-3-[(3-chloro-2-methoxyphenyl)amino]-1H,5H,6H,7H-pyrrolo[3,2-c]pyridin-4-one (18.20 mg, 32.34%) as a yellow solid.

LC-MS: (M+H)⁺ found 528.0.

¹H NMR (400 MHz, DMSO-d6) δ 10.96 (s, 1H), 8.49 (d, J=11.2 Hz, 1H), 8.25 (s, 1H), 7.28 (q, J=5.6 Hz, 2H), 6.61 (d, J=22.5 Hz, 3H), 6.14 (m, 1H), 4.85 (s, 1H), 3.85 (d, J=12.3 Hz, 3H), 3.64 (s, 1H), 3.44 (q, J=7.1 Hz, 3H), 2.78 (s, 2H), 2.26 (d, J=26.0 Hz, 1H), 2.13-2.00 (m, 2H), 1.95 (s, 4H).

### Example 196. 3-[(3-fluoro-2-methoxyphenyl)amino]-2-(3-{[(2S)-1-(prop-2-enoyl)pyrrolidin-2-yl]methoxy}pyridine-4-yl)-1H,5H,6H,7H-pyrrolo[3,2-c]pyridin-4-one (compound 212)

### 196.1. Synthesis of tert-butyl (2S)-2-{[(4-bromopyridin-3-yl)oxy]methyl}pyrrolidine-1-carboxylate

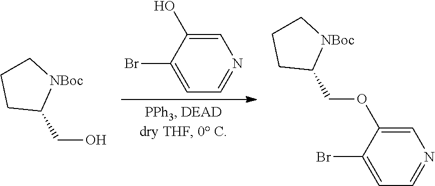

To a stirred mixture of tert-butyl (2S)-2-(hydroxymethyl)pyrrolidine-1-carboxylate (1.39 g, 6.90 mmol, 1.20 equiv.) and 4-bromopyridin-3-ol (1.00 g, 5.75 mmol, 1.00 equiv.) in THF were added PPh₃ (2.26 g, 8.62 mmol, 1.50 equiv.) at 0° C. under N₂ atmosphere, and stirred for 30 min then DEAD (1.50 g, 8.62 mmol, 1.50 equiv.) was added in portions at 0° C. under nitrogen atmosphere and stirred overnight. Concentrated under reduced pressure. The crude product was purified by Prep-HPLC with the following conditions (column: C18; mobile phase, ACN in water, 10% to 70% gradient in 30 min; detector, UV 254 nm) to afford tert-butyl (2S)-2-{[(4-bromopyridin-3-yl)oxy]methyl}pyrrolidine-1-carboxylate (1.70 g, 82.80%) as a Brown yellow oil.

LC-MS: (M+H)⁺ found 357.0.

### 196.2. Synthesis of tert-butyl (2S)-2-{[(4-{4-oxo-1H,5H,6H,7H-pyrrolo[3,2-c]pyridin-2-yl}pyridin-3-yl)oxy]methyl}pyrrolidine-1-carboxylate

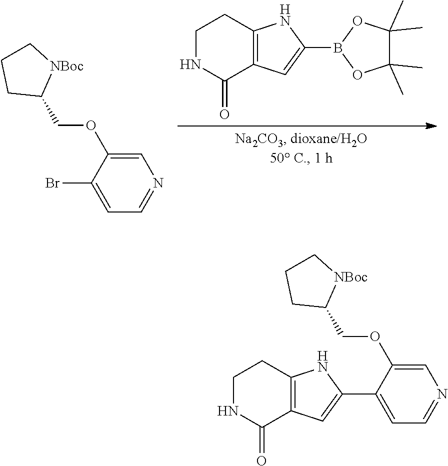 [SMILES: CC(C)(C)OC(=O)N1CCC[C@H]1COc1cnccc1Br ; CC1(C)OB(c2cc3c([nH]2)CCNC3=O)OC1(C)C ; "Na2CO3, dioxane/H2O50° C., 1 h" ; CC(C)(C)OC(=O)N1CCC[C@H]1COc1cnccc1-c1cc2c([nH]1)CCNC2=O]

Into a 100 mL round-bottom flask were added tert-butyl (2S)-2-{[(4-bromopyridin-3-yl)oxy]methyl}pyrrolidine-1-carboxylate (500.00 mg, 1.40 mmol, 1.00 equiv.), 2-(4,4,5,5-tetramethyl-1,3,2-dioxaborolan-2-yl)-1H,5H,6H,7H-pyrrolo[3,2-c]pyridin-4-one (477.00 mg, 1.82 mmol, 1.30 equiv.), Cs₂CO₃ (1.37 g, 4.20 mmol, 3.00 equiv.), XPhos palladium(II) biphenyl-2-amine chloride (110.00 mg, 0.14 mmol, 0.10 equiv.), dioxane (5 mL) and H₂O (1 mL) at 50° C. Then concentrated and the residue was purified by reverse phase flash with the following conditions (column: C18; mobile phase, ACN in water, 10% to 80% gradient in 30 min; detector, UV 254 nm) to afford tert-butyl (2S)-2-{[(4-{4-oxo-1H,5H,6H,7H-pyrrolo[3,2-c]pyridin-2-yl}pyridin-3-yl)oxy]methyl}pyrrolidine-1-carboxylate (400.00 mg, 69.29%) as a brown solid.

LC-MS: (M+H)⁺ found 413.2.

### 196.3. Synthesis of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate

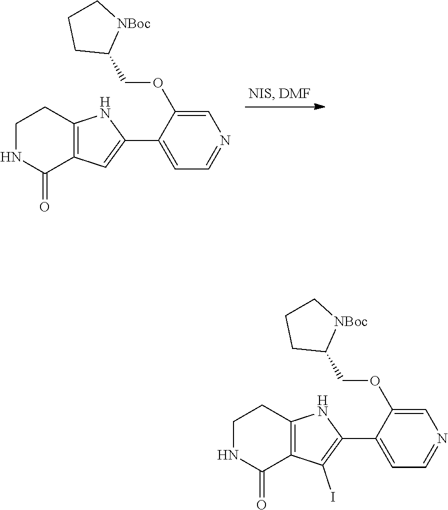 [SMILES: CC(C)(C)OC(=O)N1CCC[C@H]1COc1cnccc1-c1cc2c([nH]1)CCNC2=O ; CC(C)(C)OC(=O)N1CCC[C@H]1COc1cnccc1-c1[nH]c2c(c1I)C(=O)NCC2]

A solution of tert-butyl (2S)-2-{[(4-{4-oxo-1H,5H,6H,7H-pyrrolo[3,2-c]pyridin-2-yl}pyridin-3-yl)oxy]methyl}pyrrolidine-1-carboxylate (510.00 mg, 1.24 mmol, 1.00 equiv.) and NIS (278.17 mg, 1.24 mmol, 1.00 equiv.) in DMF (5 mL) was stirred for overnight at room temperature under nitrogen atmosphere. The reaction was monitored by LCMS. The residue was purified by reverse flash chromatography with the following conditions (column: C18 gel; mobile phase, ACN in water, 10% to 50% gradient in 10 min; detector, UV 254 nm) to afford tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (500.00 mg, 75.11%) as a yellow solid.

LC-MS: (M+H)⁺ found 539.0.

### 196.4. Synthesis of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate

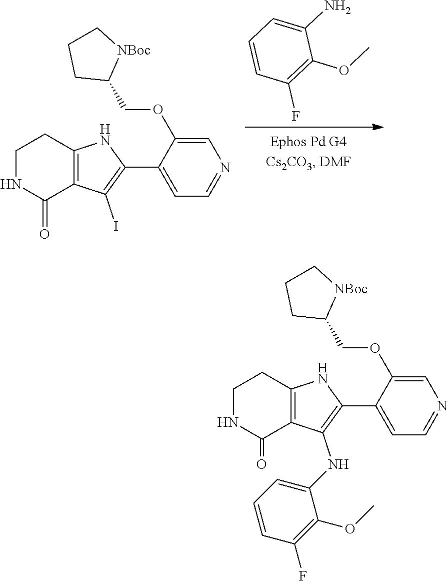

To a stirred mixture of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (100.00 mg, 0.19 mmol, 1.00 equiv.) and 3-fluoro-2-methoxyaniline (39.00 mg, 0.28 mmol, 1.50 equiv.) in DMF (1 mL) were added Ephos Pd G4 (17.00 mg, 0.02 mmol, 0.10 equiv.) and Cs₂CO₃ (121.00 mg, 0.37 mmol, 2.00 equiv.) under Argon atmosphere. The resulting suspension was backfilled with Argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (100.00 mg, 97.60%) as a yellow solid.

LC-MS: (M+H)⁺ found 552.1.

### 196.5. Synthesis of 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one

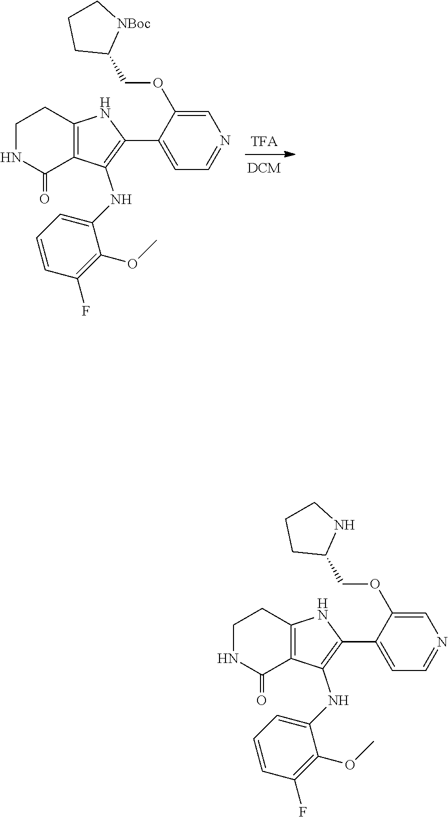 [SMILES: COc1c(F)cccc1Nc1c(-c2ccncc2OC[C@@H]2CCCN2C(=O)OC(C)(C)C)[nH]c2c1C(=O)NCC2 ; COc1c(F)cccc1Nc1c(-c2ccncc2OC[C@@H]2CCCN2)[nH]c2c1C(=O)NCC2]

To a stirred mixture of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (100.00 mg, 0.18 mmol, 1.00 equiv.) in DCM (3 mL) was added TFA (1 mL) at room temperature under nitrogen atmosphere. The resulting mixture was stirred for 1 h and concentrated under reduced pressure to afford 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (210.00 mg, crude) as a brown oil.

LC-MS: (M+H)⁺ found 452.1.

### 196.6. Synthesis of 3-[(3-fluoro-2-methoxyphenyl)amino]-2-(3-{[(2S)-1-(prop-2-enoyl)pyrrolidin-2-yl]methoxy}pyridine-4-yl)-1H,5H,6H,7H-pyrrolo[3,2-c]pyridin-4-one

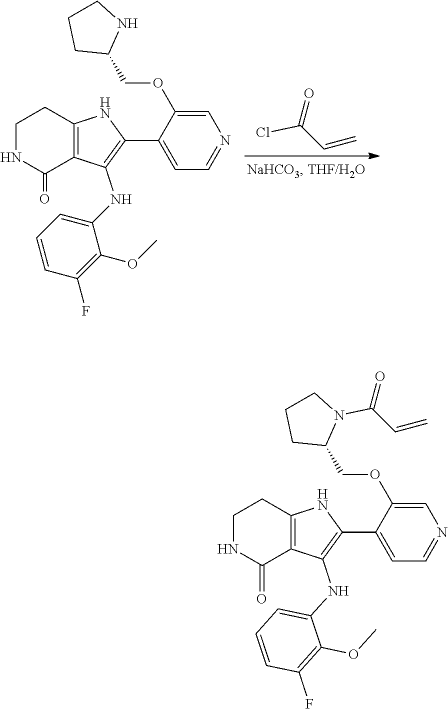

To a stirred solution of 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (180.00 mg, 0.15 mmol, 1.00 equiv.) in NaHCO₃(sat.) (1.50 mL) and THF (1.50 mL) was added acryloyl chloride (12.34 mg, 0.14 mmol, 0.90 equiv.) dropwise at 0° C. under nitrogen atmosphere. The resulting mixture was stirred for 1 h at room temperature under nitrogen atmosphere. The reaction was monitored by LCMS. The resulting mixture was concentrated under reduced pressure. The residue was purified by silica gel column chromatography, eluted with CH₂Cl₂/MeOH (20:1) to afford crude product (70.00 mg). The crude product (70.00 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 19% B to 42% B in 9 min, 42% B; Wave Length: 254/220 nm; RT1(min): 9.67; Number Of Runs: 0) to afford 3-[(3-fluoro-2-methoxyphenyl)amino]-2-(3-{[(2S)-1-(prop-2-enoyl)pyrrolidin-2-yl]methoxy}pyridine-4-yl)-1H,5H,6H,7H-pyrrolo[3,2-c]pyridin-4-one (12.30 mg, 15.63%) as a light yellow solid.

LC-MS: (M+H)⁺ found 506.0.

¹H NMR (400 MHz, Chloroform-d) δ 11.49 (s, 1H), 8.21 (s, 1H), 7.97 (d, J=5.3 Hz, 1H), 7.58 (s, 1H), 7.55 (d, J=5.3 Hz, 1H), 6.62-6.37 (m, 4H), 6.07 (d, J=8.1 Hz, 1H), 5.78 (d, J=10.3 Hz, 1H), 5.20 (s, 1H), 5.11-4.97 (m, 1H), 4.26 (t, J=9.6 Hz, 1H), 4.14-4.08 (m, 4H), 3.76 (t, J=6.7 Hz, 2H), 3.66-3.54 (m, 2H), 3.21 (t, J=6.8 Hz, 2H), 2.25-1.95 (m, 4H).

### Example 197. 3-[(3-chloro-2-methoxyphenyl)amino]-2-(3-{2-[(2R)-1-[(2E)-4-(pyrrolidin-1-yl)but-2-enoyl] pyrrolidin-2-yl]ethynyl}pyridin-4-yl)-1H,5H,6H,7H-pyrrolo[3,2-c]pyridin-4-one (compound 229)

### 197.1. Synthesis of (R,E)-2-(3-((1-(4-bromobut-2-enoyl)pyrrolidin-2-yl)ethynyl)pyridin-4-yl)-3-((3-chloro-2-methoxyphenyl)amino)-1,5,6,7-tetrahydro-4H-pyrrolo[3,2-c]pyridin-4-one

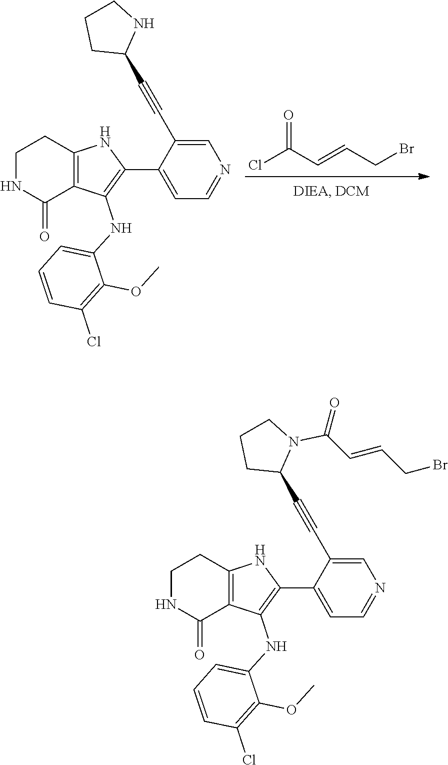

To a stirred solution of 3-[(3-chloro-2-methoxyphenyl)amino]-2-(3-{2-[(2R)-pyrrolidin-2-yl]ethynyl}pyridin-4-yl)-1H,5H,6H,7H-pyrrolo[3,2-c]pyridin-4-one (40.00 mg, 0.08 mmol, 1.00 equiv.) and DIEA (34.00 mg, 0.26 mmol, 3.00 equiv.) in DCM (2.50 mL) were added (2E)-4-bromobut-2-enoyl chloride (19.00 mg, 0.10 mmol, 1.2 equiv.) dropwise at 0° C. under nitrogen atmosphere. The resulting mixture was stirred for 0.5 h at 0° C. under nitrogen atmosphere. The resulting mixture was used in the next step directly without further purification.

LCMS: (M+H)⁺ found 608.0 and 564.0.

### 197.2. Synthesis of 3-[(3-chloro-2-methoxyphenyl)amino]-2-(3-{2-[(2R)-1-[(2E)-4-(pyrrolidin-1-yl)but-2-enoyl] pyrrolidin-2-yl]ethynyl}pyridin-4-yl)-1H,5H,6H,7H-pyrrolo[3,2-c]pyridin-4-one

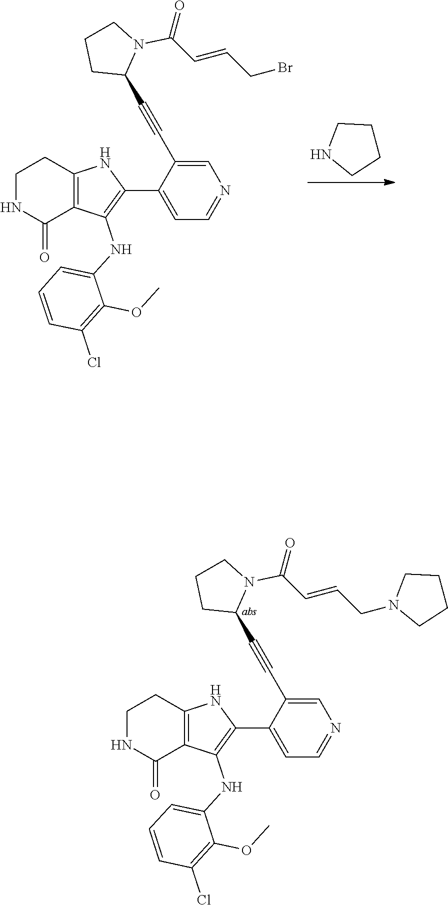 [SMILES: COc1c(Cl)cccc1Nc1c(-c2ccncc2C#C[C@H]2CCCN2C(=O)/C=C/CBr)[nH]c2c1C(=O)NCC2 ; C1CCNC1 ; COc1c(Cl)cccc1Nc1c(-c2ccncc2C#C[C@H]2CCCN2C(=O)/C=C/CN2CCCC2)[nH]c2c1C(=O)NCC2]

To the last step resulting mixture was added pyrrolidine (19.00 mg, 0.26 mmol, 3.00 equiv.) dropwise at 0° C. The resulting mixture was stirred for additional 48 h at room temperature. The resulting mixture was extracted with CH₂Cl₂:MeOH (3×50 mL). The combined organic layers were dried over anhydrous Na₂SO₄. After filtration, the filtrate was concentrated under reduced pressure and dissolved in DMSO. The crude product (100.00 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 10% B to 28% B in 8 min, 28% B; Wave Length: 254/220 nm; RT1(min): 8; Number Of Runs: 0) to afford 3-[(3-chloro-2-methoxyphenyl)amino]-2-(3-{2-[(2R)-1-[(2E)-4-(pyrrolidin-1-yl)but-2-enoyl] pyrrolidin-2-yl]ethynyl}pyridin-4-yl)-1H,5H,6H,7H-pyrrolo[3,2-c]pyridin-4-one (12.60 mg, 24.29%) as a yellow solid.

LC-MS: (M+H)⁺ found 599.6.

¹H NMR (400 MHz, Chloroform-d) δ 11.12 (s, 1H), 8.56 (s, 1H), 8.17 (d, J=5.6 Hz, 1H), 7.72 (s, 1H), 7.44 (d, J=5.6 Hz, 1H), 6.97 (m, J=15.2, 6.4 Hz, 1H), 6.73 (m, J=8.0, 1.5 Hz, 1H), 6.62 (t, J=8.1 Hz, 1H), 6.52 (m, J=15.0, 1.5 Hz, 1H), 6.24 (m, J=8.2, 1.5 Hz, 1H), 5.52 (s, 1H), 4.91 (m, J=7.3, 4.6 Hz, 1H), 4.09 (s, 3H), 3.82 (m, J=10.0, 6.4 Hz, 1H), 3.73-3.54 (m, 5H), 3.27 (t, J=6.5 Hz, 2H), 2.91 (d, J=6.4 Hz, 4H), 2.40-2.21 (m, 3H), 2.17-2.05 (m, 1H), 1.97 (p, J=3.7 Hz, 4H).

### Example 198. 3-[(3-chloro-2-methoxyphenyl)amino]-2-(3-{2-[(2R)-1-[(2E)-4-(morpholin-4-yl)but-2-enoyl]pyrrolidin-2-yl]ethynyl}pyridin-4-yl)-1H,5H,6H,7H-pyrrolo[3,2-c]pyridin-4-one (compound 228)

### 198.1. Synthesis of (R,E)-2-(3-((1-(4-bromobut-2-enoyl)pyrrolidin-2-yl)ethynyl)pyridin-4-yl)-3-((3-chloro-2-methoxyphenyl)amino)-1,5,6,7-tetrahydro-4H-pyrrolo[3,2-c]pyridin-4-one

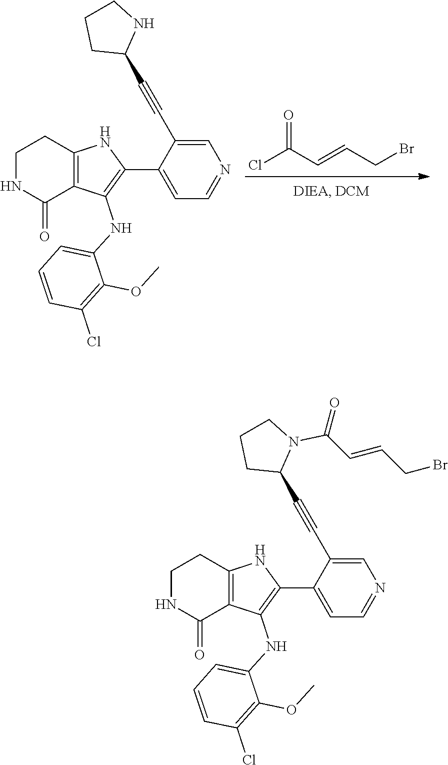

To a stirred solution of 3-[(3-chloro-2-methoxyphenyl)amino]-2-(3-{2-[(2R)-pyrrolidin-2-yl]ethynyl}pyridin-4-yl)-1H,5H,6H,7H-pyrrolo[3,2-c]pyridin-4-one (40.00 mg, 0.08 mmol, 1.00 equiv.) and DIEA (34.00 mg, 0.26 mmol, 3.00 equiv.) in DCM (2.50 mL) were added (2E)-4-bromobut-2-enoyl chloride (19.00 mg, 0.10 mmol, 1.20 equiv.) dropwise at 0° C. under nitrogen atmosphere. The resulting mixture was stirred for 0.5 h at 0° C. under nitrogen atmosphere. The resulting mixture was used in the next step directly without further purification.

LCMS: (M+H)⁺ found 608.0 and 564.0.

### 198.2. Synthesis of 3-[(3-chloro-2-methoxyphenyl)amino]-2-(3-{2-[(2R)-1-[(2E)-4-(morpholin-4-yl)but-2-enoyl]pyrrolidin-2-yl]ethynyl}pyridin-4-yl)-1H,5H,6H,7H-pyrrolo[3,2-c]pyridin-4-one

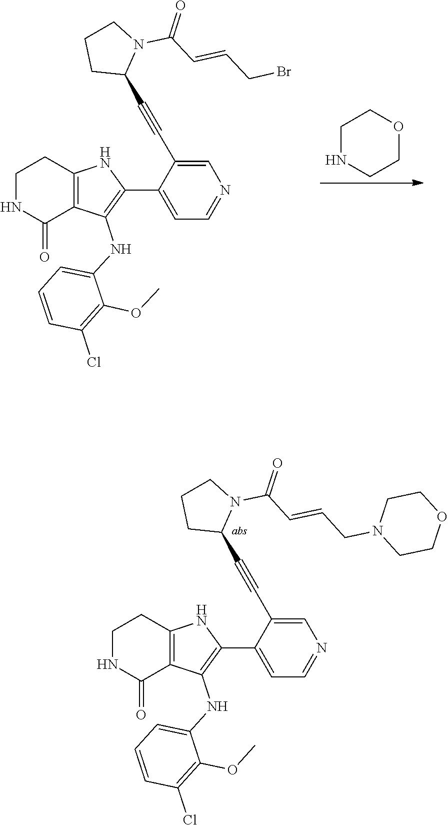 [SMILES: COc1c(Cl)cccc1Nc1c(-c2ccncc2C#C[C@H]2CCCN2C(=O)/C=C/CBr)[nH]c2c1C(=O)NCC2 ; C1COCCN1 ; COc1c(Cl)cccc1Nc1c(-c2ccncc2C#C[C@H]2CCCN2C(=O)/C=C/CN2CCOCC2)[nH]c2c1C(=O)NCC2]

To the last step resulting mixture was added morpholine (23.00 mg, 0.26 mmol, 3.00 equiv.) dropwise at 0° C. The resulting mixture was stirred for additional 48 h at room temperature. The resulting mixture was extracted with CH₂Cl₂:MeOH (3×50 mL). The combined organic layers were dried over anhydrous Na₂SO₄. After filtration, the filtrate was concentrated under reduced pressure and dissolved in DMSO. The crude product (100.00 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 10% B to 27% B in 8 min, 27% B; Wave Length: 254/220 nm; RT1(min): 8; Number Of Runs: 0) to afford 3-[(3-chloro-2-methoxyphenyl)amino]-2-(3-{2-[(2R)-1-[(2E)-4-(morpholin-4-yl)but-2-enoyl]pyrrolidin-2-yl]ethynyl}pyridin-4-yl)-1H,5H,6H,7H-pyrrolo[3,2-c]pyridin-4-one (16.40 mg, 30.79%) as a yellow solid.

LC-MS: (M+H)⁺ found 615.2.

¹H NMR (400 MHz, Chloroform-d) δ 11.14 (s, 1H), 8.56 (s, 1H), 8.22-8.13 (m, 1H), 7.72 (s, 1H), 7.44 (d, J=5.6 Hz, 1H), 6.95 (m, J=15.1, 6.3 Hz, 1H), 6.73 (m, J=8.1, 1.5 Hz, 1H), 6.62 (t, J=8.1 Hz, 1H), 6.40 (d, J=15.1 Hz, 1H), 6.23 (m, J=8.1, 1.5 Hz, 1H), 5.64 (t, J=2.5 Hz, 1H), 4.92 (m, J=7.2, 4.6 Hz, 1H), 4.09 (s, 3H), 3.89-3.52 (m, 9H), 3.34-3.17 (m, 4H), 2.55 (d, J=4.8 Hz, 3H), 2.44-2.21 (m, 3H), 2.20-2.04 (m, 1H).

### Example 199. 3-[(3-chloro-2-methoxyphenyl)amino]-2-(3-{[(2R)-1-[(2E)-4-[(3S)-3-methoxypyrrolidin-1-yl]but-2-enoyl]azetidin-2-yl]methoxy}pyridin-4-yl)-1H,5H,6H,7H-pyrrolo[3,2-c]pyridin-4-one (compound 222)

### 199.1. Synthesis of (R,E)-2-(3-((1-(4-bromobut-2-enoyl)azetidin-2-yl)methoxy)pyridin-4-yl)-3-((3-chloro-2-methoxyphenyl)amino)-1,5,6,7-tetrahydro-4H-pyrrolo[3,2-c]pyridin-4-one

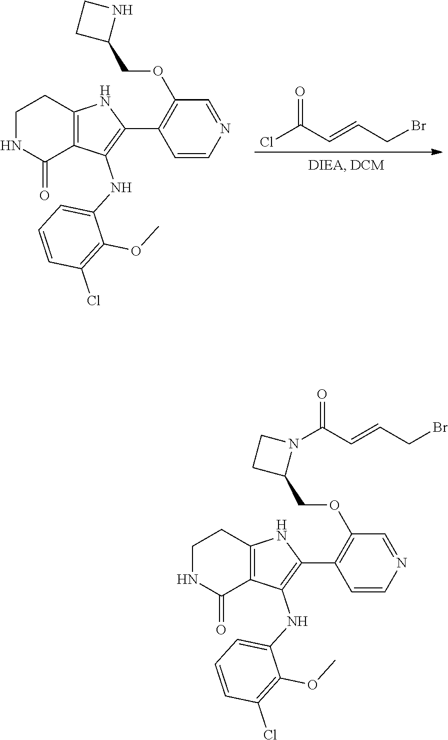 [SMILES: COc1c(Cl)cccc1Nc1c(-c2ccncc2OC[C@H]2CCN2)[nH]c2c1C(=O)NCC2 ; O=C(Cl)/C=C/CBr ; COc1c(Cl)cccc1Nc1c(-c2ccncc2OC[C@H]2CCN2C(=O)/C=C/CBr)[nH]c2c1C(=O)NCC2]

To a stirred mixture of 2-{3-[(2R)-azetidin-2-ylmethoxy]pyridin-4-yl}-3-[(3-chloro-2-methoxyphenyl) amino]-1H,5H,6H,7H-pyrrolo[3,2-c]pyridin-4-one (100.00 mg, 0.22 mmol, 1.00 equiv.) and DIEA (85.00 mg, 0.66 mmol, 3.00 equiv.) in DCM (2 mL) was added (2E)-4-bromobut-2-enoyl chloride (48.00 mg, 0.26 mmol, 1.20 equiv.) dropwise at 0° C. The resulting mixture was stirred for 2 h at 25° C. Desired product could be detected by LCMS. The resulting mixture was used in the next step directly without further purification.

LC-MS: (M+H)⁺ found 600.0.

### 199.2. Synthesis of 3-[(3-chloro-2-methoxyphenyl)amino]-2-(3-{[(2R)-1-[(2E)-4-[(3S)-3-methoxypyrrolidin-1-yl]but-2-enoyl]azetidin-2-yl]methoxy}pyridin-4-yl)-1H,5H,6H,7H-pyrrolo[3,2-c]pyridin-4-one

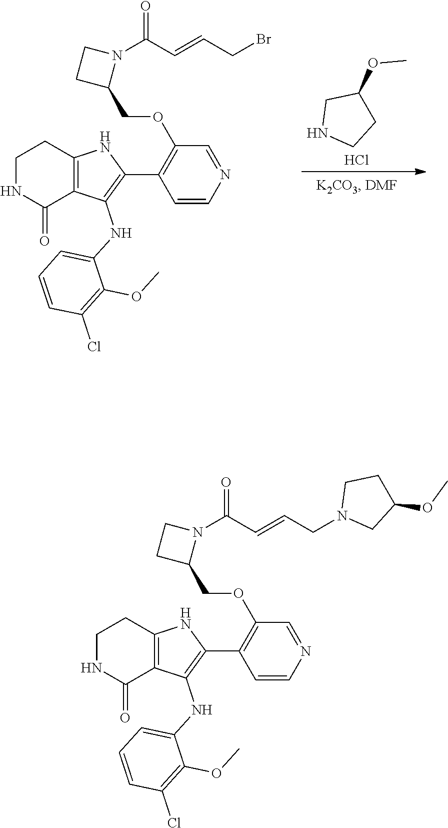

To a solution of (3S)-3-methoxypyrrolidine hydrochloride (36.00 mg, 0.26 mmol, 1.20 equiv.) in DMF (1 mL) was added K₂CO₃ (90.00 mg, 0.65 mmol, 3.00 equiv.) and the mixture was stirred for 30 min. Then the mixture was added to the solution of 2-(3-{[(2R)-1-[(2E)-4-bromobut-2-enoyl]azetidin-2-yl]methoxy}pyridin-4-yl)-3-[(3-chloro-2-methoxyphenyl)amino]-1H,5H,6H,7H-pyrrolo[3,2-c]pyridin-4-one (130.00 mg, 0.22 mmol, 1.00 equiv.) in 2 mL DCM and stirred for 4 h at room temperature. Desired product could be detected by LCMS. The reaction mixture was quenched by water and extracted with DCM (3*10 mL). The resulting mixture was concentrated under reduced pressure. The crude product was purified by Prep-HPLC with the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 23% B to 53% B in 8 min, 53% B; Wave Length: 220/254 nm; RT1(min): 7.25; Number Of Runs: 0) to afford 3-[(3-chloro-2-methoxyphenyl)amino]-2-(3-{[(2R)-1-[(2E)-4-[(3 S)-3-methoxypyrrolidin-1-yl]but-2-enoyl]azetidin-2-yl]methoxy}pyridin-4-yl)-1H,5H,6H,7H-pyrrolo[3,2-c]pyridin-4-one (29.50 mg, 21.78%) as a yellow solid.

LC-MS: (M+H)⁺ found 621.2.

¹H NMR (300 MHz, Chloroform-d) δ 11.77 (s, 1H), 8.15 (s, 1H), 7.92 (d, J=5.2 Hz, 1H), 7.46 (s, 1H), 7.38 (d, J=5.2 Hz, 1H), 7.01-6.81 (m, 1H), 6.68-6.47 (m, 2H), 6.22-5.98 (m, 2H), 5.22 (s, 1H), 5.02-4.86 (m, 1H), 4.42 (t, J=9.8 Hz, 1H), 4.29-4.10 (m, 3H), 4.01 (s, 3H), 3.93-3.81 (m, 1H), 3.58-3.46 (m, 2H), 3.34-3.16 (m, 5H), 3.14-2.95 (m, 2H), 2.79-2.39 (m, 5H), 2.10-1.96 (m, 2H), 1.88-1.72 (m, 1H).

### Example 200. 3-[(3-chloro-2-methoxyphenyl)amino]-2-(3-{[(2R)-1-[(2E)-4-(morpholin-4-yl)but-2-enoyl]azetidin-2-yl]methoxy}pyridine-4-yl)-1H,5H,6H,7H-pyrrolo[3,2-c]pyridin-4-one (compound 220)

### 200.1. Synthesis of (R,E)-2-(3-((1-(4-bromobut-2-enoyl)azetidin-2-yl)methoxy)pyridin-4-yl)-3-((3-chloro-2-methoxyphenyl)amino)-1,5,6,7-tetrahydro-4H-pyrrolo[3,2-c]pyridin-4-one

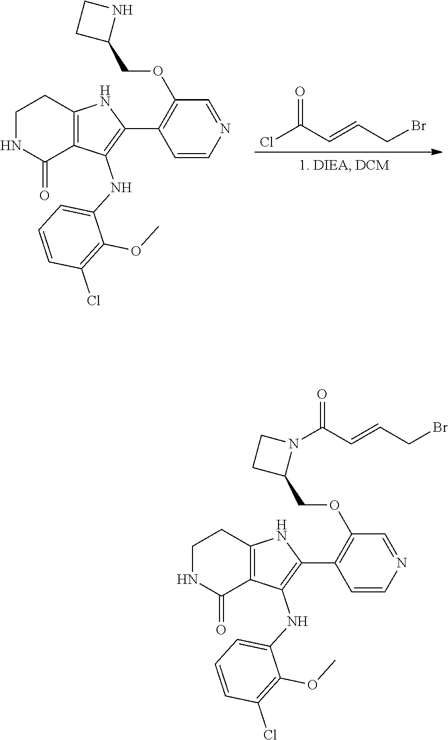 [SMILES: COc1c(Cl)cccc1Nc1c(-c2ccncc2OC[C@H]2CCN2)[nH]c2c1C(=O)NCC2 ; O=C(Cl)/C=C/CBr ; COc1c(Cl)cccc1Nc1c(-c2ccncc2OC[C@H]2CCN2C(=O)/C=C/CBr)[nH]c2c1C(=O)NCC2]

To a stirred solution of 2-{3-[(2R)-azetidin-2-ylmethoxy]pyridin-4-yl}-3-[(3-chloro-2-methoxyphenyl) amino]-1H,5H,6H,7H-pyrrolo[3,2-c]pyridin-4-one (60.00 mg, 0.13 mmol, 1.00 equiv.) and DIEA (51.00 mg, 0.39 mmol, 3.00 equiv.) in DCM (3 mL) were added (2E)-4-bromobut-2-enoyl chloride (29.00 mg, 0.15 mmol, 1.20 equiv.) dropwise at 0° C. under nitrogen atmosphere. The resulting mixture was stirred for 0.5 h at 0° C. under nitrogen atmosphere. The resulting mixture was used in the next step directly without further purification.

LCMS: (M+H)⁺ found 600.0 and 556.0.

### 200.2. Synthesis of 3-[(3-chloro-2-methoxyphenyl)amino]-2-(3-{[(2R)-1-[(2E)-4-(morpholin-4-yl)but-2-enoyl]azetidin-2-yl]methoxy}pyridine-4-yl)-1H,5H,6H,7H-pyrrolo[3,2-c]pyridin-4-one

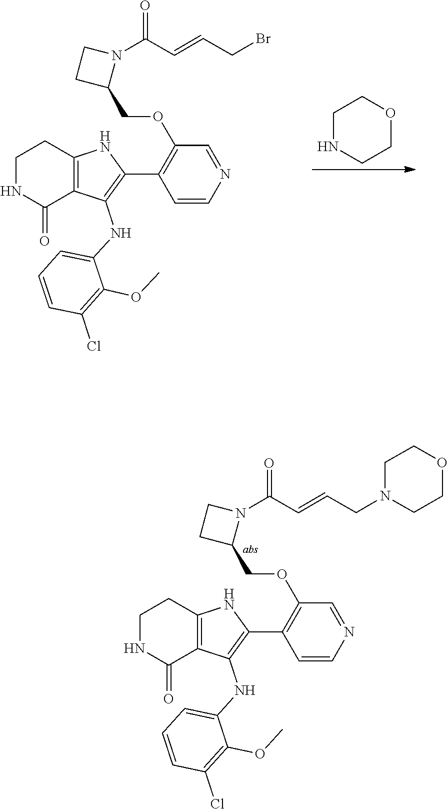 [SMILES: COc1c(Cl)cccc1Nc1c(-c2ccncc2OC[C@H]2CCN2C(=O)/C=C/CBr)[nH]c2c1C(=O)NCC2 ; C1COCCN1 ; COc1c(Cl)cccc1Nc1c(-c2ccncc2OC[C@H]2CCN2C(=O)/C=C/CN2CCOCC2)[nH]c2c1C(=O)NCC2]

To the last step resulting mixture was added morpholine (35.00 mg, 0.39 mmol, 3.00 equiv.) dropwise at 0° C. The resulting mixture was stirred for additional overnight at room temperature. Desired product could be detected by LCMS. The resulting mixture was extracted with CH₂Cl₂:MeOH (3×50 mL). The combined organic layers were dried over anhydrous Na₂SO₄. After filtration, the filtrate was concentrated under reduced pressure and dissolved in DMSO. The crude product (100.00 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 24% B to 54% B in 7 min; Wave Length: 254 nm; RT1(min): 6.5; Number Of Runs: 0) to afford 3-[(3-chloro-2-methoxyphenyl)amino]-2-(3-{[(2R)-1-[(2E)-4-(morpholin-4-yl)but-2-enoyl]azetidin-2-yl]methoxy}pyridine-4-yl)-1H,5H,6H,7H-pyrrolo[3,2-c]pyridin-4-one (27.10 mg, 33.77%) as an off-white solid.

LC-MS: (M+H)⁺ found 607.2.

¹H NMR (400 MHz, Chloroform-d) δ 11.82 (s, 1H), 8.26 (s, 1H), 7.99 (d, J=5.3 Hz, 1H), 7.64 (s, 1H), 7.47 (d, J=5.3 Hz, 1H), 6.94 (m, J=15.3, 6.3 Hz, 1H), 6.74 (m, J=8.1, 1.5 Hz, 1H), 6.63 (t, J=8.1 Hz, 1H), 6.28-6.07 (m, 2H), 5.26 (d, J=2.6 Hz, 1H), 5.04 (q, J=8.5 Hz, 1H), 4.53 (t, J=9.8 Hz, 1H), 4.41-4.18 (m, 3H), 4.11 (s, 3H), 3.76 (t, J=4.6 Hz, 4H), 3.63 (m, J=7.0, 6.3, 2.2 Hz, 2H), 3.33-3.04 (m, 4H), 2.65 (m, J=12.0, 9.4, 6.0 Hz, 1H), 2.52 (t, J=4.7 Hz, 4H), 2.19-2.11 (m, 1H).

### Example 201. rel-2-(3-{[(2R)-1-[(2E)-4-(dimethylamino)but-2-enoyl]pyrrolidin-2-yl]methoxy}pyridin-4-yl)-3-[(3-fluoro-2-methoxyphenyl)amino]-1H,5H,6H,7H-pyrrolo[3,2-c]pyridin-4-one (compound 283)

### 201.1. Synthesis of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate

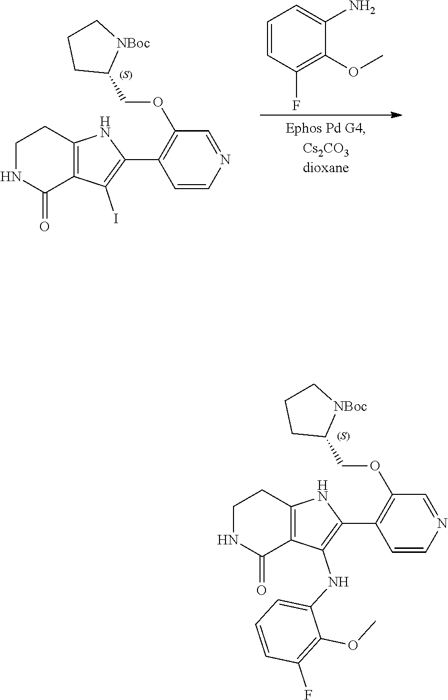

To a stirred mixture of tert-butyl (2S)-2-{[(4-{3-iodo-4-oxo-1H,5H,6H,7H-pyrrolo[3,2-c]pyridin-2-yl}pyridin-3-yl)oxy]methyl}pyrrolidine-1-carboxylate (400.00 mg, 0.74 mmol, 1.00 equiv.) and 3-fluoro-2-methoxyaniline (210.00 mg, 1.49 mmol, 2.00 equiv.) in DMF (5 mL) were added EPhos Pd G4 (68.00 mg, 0.07 mmol, 0.10 equiv.) and Cs₂CO₃ (484.00 mg, 1.49 mmol, 2.00 equiv.) under Argon atmosphere. The resulting suspension was backfilled with Argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (310.00 mg, 75.64%) as a brown solid.

LC-MS: (M+H)⁺ found 552.0.

### 201.2. Synthesis of 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one

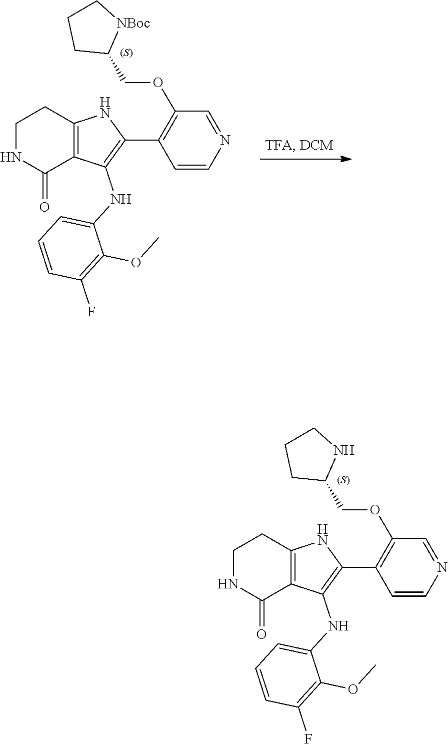

To a stirred mixture of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}pyrrolidine-1-carboxylate (290.00 mg, 0.53 mmol, 1.00 equiv.) in DCM (3 mL) was added TFA (1 mL) at room temperature under nitrogen atmosphere. The resulting mixture was stirred for 1 h and concentrated under reduced pressure to afford 3-[(3-fluoro-2-methoxyphenyl)amino]-2-{3-[(2S)-pyrrolidin-2-ylmethoxy]pyridin-4-yl}-1H,5H,6H,7H-pyrrolo[3,2-c]pyridin-4-one (540.00 mg, crude) as a red oil.

LC-MS: (M+H)⁺ found 452.0.

### 201.3. Synthesis of rel-2-(3-{[(2R)-1-[(2E)-4-(dimethylamino)but-2-enoyl]pyrrolidin-2-yl]methoxy}pyridin-4-yl)-3-[(3-fluoro-2-methoxyphenyl)amino]-1H,5H,6H,7H-pyrrolo[3,2-c]pyridin-4-one

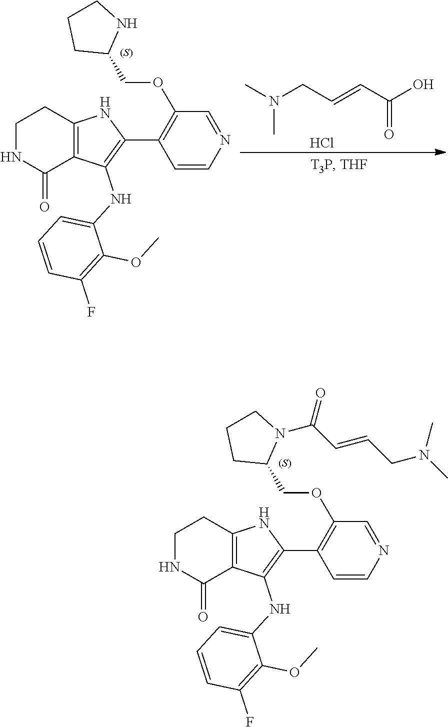

A solution of rel-3-[(3-fluoro-2-methoxyphenyl)amino]-2-{3-[(2R)-pyrrolidin-2-ylmethoxy]pyridin-4-yl}-1H,5H,6H,7H-pyrrolo[3,2-c]pyridin-4-one (60.00 mg, 0.13 mmol, 1.00 equiv.) in THF (2 mL) was basified to pH 8 with DIEA. To the above mixture was added (2E)-4-(dimethylamino)but-2-enoic acid (26.00 mg, 0.20 mmol, 1.50 equiv.) at 0° C. under nitrogen atmosphere followed by the addition of T₃P (85.00 mg, 0.27 mmol, 2.00 equiv.) dropwise. The resulting mixture was stirred for 1 h at room temperature under nitrogen atmosphere. Saturated aqueous NaHCO₃(10 mL) was added to the reaction mixture at 0° C. and extracted with EtOAc (3×10 mL), dried over anhydrous Na₂SO₄ and filtered. The filtrate was concentrated under reduced pressure to give crude product (50.00 mg) that was purified by Prep-HPLC under the following conditions (Column: YMC-ActusTriArt C18 ExRS, 30*150 mm, 5 μm; Mobile Phase A: Water (10 mmol/L NH₄HCO₃), Mobile Phase B: ACN; Flow rate: 60 mL/min; Gradient: 20% B to 42% B in 10 min, 42% B; Wave Length: 254/220 nm; RT1(min): 8.85; Number Of Runs: 0) to afford rel-2-(3-{[(2R)-1-[(2E)-4-(dimethylamino)but-2-enoyl]pyrrolidin-2-yl]methoxy}pyridin-4-yl)-3-[(3-fluoro-2-methoxyphenyl)amino]-1H,5H,6H,7H-pyrrolo[3,2-c]pyridin-4-one (7.50 mg, 9.96%) as a white solid.

LC-MS: (M+H)⁺ found 563.2.

¹H NMR (300 MHz, Chloroform-d) δ 11.51 (s, 1H), 8.23 (s, 1H), 8.01 (d, J=5.3 Hz, 1H), 7.56 (d, J=3.9 Hz, 2H), 6.97 (d, J=6.2 Hz, 1H), 6.68-6.42 (m, 3H), 6.09 (d, J=8.1 Hz, 1H), 5.20 (s, 1H), 5.05 (s, 1H), 4.29 (t, J=9.5 Hz, 1H), 4.13 (d, J=1.2 Hz, 4H), 3.80 (t, J=6.6 Hz, 2H), 3.63 (t, J=6.8 Hz, 2H), 3.30 (d, J=6.2 Hz, 2H), 3.22 (t, J=6.8 Hz, 2H), 2.43 (s, 6H), 2.15 (d, J=8.6 Hz, 3H), 1.89 (s, 1H).

### Example 202. 3-[(3-fluoro-2-methoxyphenyl)amino]-2-(3-{[(2S)-2-methyl-1-(prop-2-enoyl)pyrrolidin-2-yl]methoxy}pyridin-4-yl)-1H,5H,6H,7H-pyrrolo[3,2-c]pyridin-4-one (compound 329)

### 202.1. Synthesis of tert-butyl (2S)-2-(hydroxymethyl)-2-methylpyrrolidine-1-carboxylate

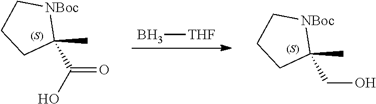

To a stirred solution of (2S)-1-(tert-butoxycarbonyl)-2-methylpyrrolidine-2-carboxylic acid (2.00 g, 8.72 mmol, 1.00 equiv.) in THF (20 mL) was added BH₃-THF (13.10 mL, 13.09 mmol, 1.50 equiv.) dropwise at 0° C. under nitrogen atmosphere. The resulting mixture was stirred for 1 h at 75° C. under nitrogen atmosphere. Desired product could be detected by TLC. The reaction was quenched with MeOH at 0° C. The resulting mixture was concentrated under reduced pressure. The residue was purified by silica gel column chromatography, eluted with PE/EA (20:1) to afford tert-butyl (2S)-2-(hydroxymethyl)-2-methylpyrrolidine-1-carboxylate (1.60 g, 85.20%) as a light yellow oil.

### 202.2. Synthesis of afford tert-butyl (2S)-2-{[(4-cyanopyridin-3-yl)oxy]methyl}-2-methylpyrrolidine-1-carboxylate

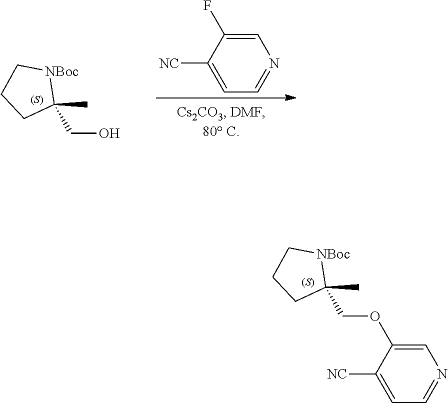

To a stirred mixture of tert-butyl (2S)-2-(hydroxymethyl)-2-methylpyrrolidine-1-carboxylate (1.20 g, 5.57 mmol, 1.00 equiv.) and 3-fluoropyridine-4-carbonitrile (681.00 mg, 5.57 mmol, 1.00 equiv.) in DMF (12 mL) were added Cs₂CO₃ (5.45 g, 16.72 mmol, 3.00 equiv.) in portions at room temperature under nitrogen atmosphere. The resulting mixture was stirred for 2 h at 80° C. under nitrogen atmosphere. Desired product could be detected by LCMS. The resulting mixture was extracted with EtOAc (3×50 mL). The combined organic layers were washed with NaCl aq. (3×100 mL), dried over anhydrous Na₂SO₄. After filtration, the filtrate was concentrated under reduced pressure. The residue was purified by silica gel column chromatography, eluted with PE/EA (5:1) to afford tert-butyl (2S)-2-{[(4-cyanopyridin-3-yl)oxy]methyl}-2-methylpyrrolidine-1-carboxylate (1.60 g, 90.44%) as a colorless oil.

LC-MS: (M+H)⁺ found 318.0.

### 202.3. Synthesis of tert-butyl (2S)-2-({[4-(aminomethyl)pyridin-3-yl]oxy}methyl)-2-methylpyrrolidine-1-carboxylate

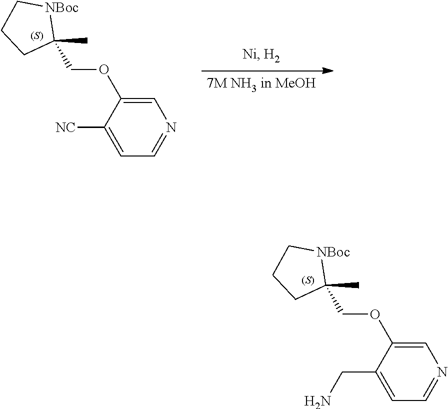

To a stirred solution of tert-butyl (2S)-2-{[(4-cyanopyridin-3-yl)oxy]methyl}-2-methylpyrrolidine-1-carboxylate (1.00 g, 3.15 mmol, 1.00 equiv.) and NH₃(g) in MeOH (10 mL) in MeOH (20 mL) was added Raney Ni (200.00 mg, 1.86 mmol, 0.59 equiv., 80%) in portions at room temperature under Argon atmosphere. The resulting mixture was stirred for 2 h at room temperature under hydrogen atmosphere. Desired product could be detected by LCMS. The resulting mixture was filtered, the filter cake was washed with MeOH (3×50 mL). The filtrate was concentrated under reduced pressure. The residue was purified by silica gel column chromatography, eluted with CH₂Cl₂/MeOH (10:1) to afford tert-butyl (2S)-2-({[4-(aminomethyl)pyridin-3-yl]oxy}methyl)-2-methylpyrrolidine-1-carboxylate (1.00 g, 98.75%) as a yellow oil.

LC-MS: (M+H)⁺ found 322.0.

### 202.4. Synthesis of tert-butyl (2S)-2-[({4-[({3-[(3-fluoro-2-methoxyphenyl)carbamothioyl]-2-oxo-5,6-dihydro-1H-pyridin-4-yl}amino)methyl]pyridin-3-yl}oxy)methyl]-2-methylpyrrolidine-1-carboxylate

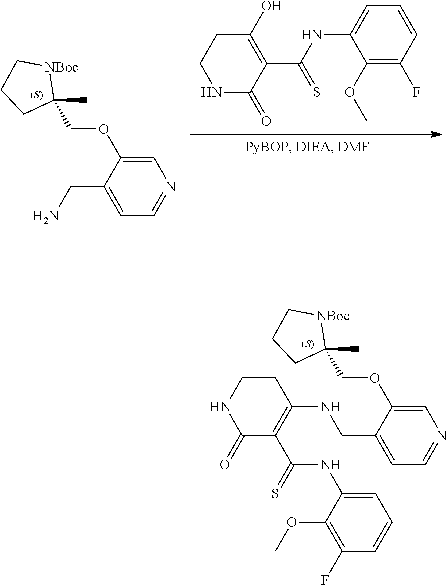

To a stirred solution of tert-butyl (2S)-2-({[4-(aminomethyl)pyridin-3-yl]oxy}methyl)-2-methylpyrrolidine-1-carboxylate (960.00 mg, 2.99 mmol, 1.00 equiv.) and N-(3-fluoro-2-methoxyphenyl)-4-hydroxy-2-oxo-5,6-dihydro-1H-pyridine-3-carbothioamide (885.00 mg, 2.99 mmol, 1.00 equiv.) in DMF (10 mL) were added DIEA (1.16 g, 8.96 mmol, 3.00 equiv.) and PyBOP (2.33 g, 4.48 mmol, 1.50 equiv.) in one portion at room temperature under nitrogen atmosphere. The resulting mixture was stirred for overnight at room temperature under nitrogen atmosphere. Desired product could be detected by LCMS. The resulting mixture was extracted with EtOAc (3×30 mL). The combined organic layers were washed with NaCl aq. (3×50 mL), dried over anhydrous Na₂SO₄. After filtration, the filtrate was concentrated under reduced pressure. The residue was purified by silica gel column chromatography, eluted with PE/EA (1:1) to afford tert-butyl (2S)-2-[({4-[({3-[(3-fluoro-2-methoxyphenyl)carbamothioyl]-2-oxo-5,6-dihydro-1H-pyridin-4-yl}amino)methyl]pyridin-3-yl}oxy)methyl]-2-methylpyrrolidine-1-carboxylate (1.00 g, 55.83%) as a yellow solid.

LC-MS: (M+H)⁺ found 600.0.

### 202.5. Synthesis of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-2-methylpyrrolidine-1-carboxylate

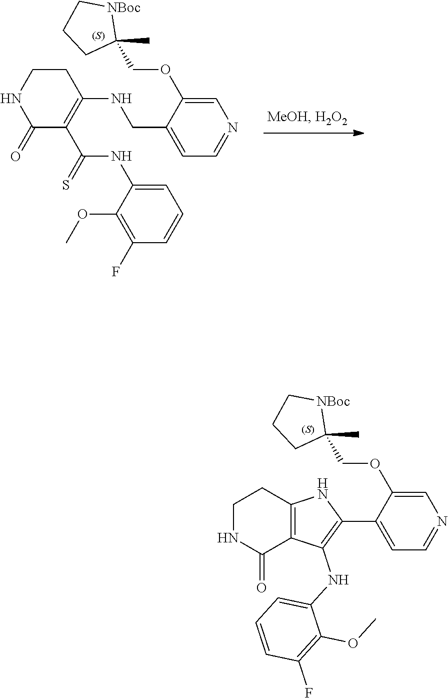

To a stirred solution of tert-butyl (2S)-2-[({4-[({3-[(3-fluoro-2-methoxyphenyl)carbamothioyl]-2-oxo-5,6-dihydro-1H-pyridin-4-yl}amino)methyl]pyridin-3-yl}oxy)methyl]-2-methylpyrrolidine-1-carboxylate (950.00 mg, 1.58 mmol, 1.00 equiv.) in MeOH (10 mL) was added H₂O₂ (234.00 mg, 2.06 mmol, 1.30 equiv., 30%) dropwise at room temperature under nitrogen atmosphere. The resulting mixture was stirred for overnight at 80° C. under nitrogen atmosphere. Desired product could be detected by LCMS. The residue was purified by reverse flash chromatography with the following conditions (column: C18 column; mobile phase, ACN in water, 0% to 60% gradient in 10 min; detector, UV 254 nm) to afford tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-2-methylpyrrolidine-1-carboxylate (240.00 mg, 26.78%) as a yellow solid.

LC-MS: (M+H)⁺ found 566.0.

### 202.6. Synthesis of 3-[(3-fluoro-2-methoxyphenyl)amino]-2-(3-{[(2S)-2-methylpyrrolidin-2-yl]methoxy}pyridin-4-yl)-1H,5H,6H,7H-pyrrolo[3,2-c]pyridin-4-one

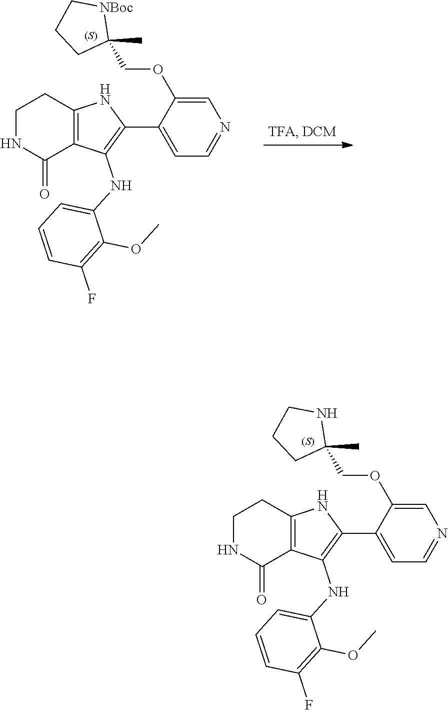

To a stirred mixture of tert-butyl (2S)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-2-methylpyrrolidine-1-carboxylate (100.00 mg, 0.18 mmol, 1.00 equiv.) in DCM (1 mL) was added TFA (0.5 mL) at room temperature under nitrogen atmosphere. The resulting mixture was stirred for 1 h and concentrated under reduced pressure to afford 3-[(3-fluoro-2-methoxyphenyl)amino]-2-(3-f{[(2S)-2-methylpyrrolidin-2-yl]methoxy}pyridin-4-yl)-1H,5H,6H,7H-pyrrolo[3,2-c]pyridin-4-one (70.00 mg, 85.05%) as a yellow solid.

LC-MS: (M+H)⁺ found 466.1.

### 202.7. Synthesis of 3-[(3-fluoro-2-methoxyphenyl)amino]-2-(3-{[(2S)-2-methyl-1-(prop-2-enoyl)pyrrolidin-2-yl]methoxy}pyridin-4-yl)-1H,5H,6H,7H-pyrrolo[3,2-c]pyridin-4-one

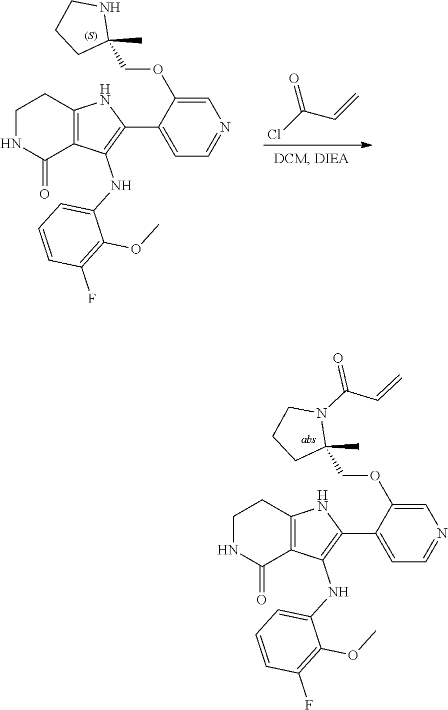

To a stirred solution of 3-[(3-fluoro-2-methoxyphenyl)amino]-2-(3-{[(2S)-2-methylpyrrolidin-2-yl]methoxy}pyridin-4-yl)-1H,5H,6H,7H-pyrrolo[3,2-c]pyridin-4-one (60.00 mg, 0.13 mmol, 1.00 equiv.) in DCM (3 mL) were added DIEA (50.00 mg, 0.39 mmol, 3.00 equiv.) and acryloyl chloride (12.00 mg, 0.13 mmol, 1.00 equiv.) dropwise at 0° C. under nitrogen atmosphere. The resulting mixture was stirred for 1 h at room temperature under nitrogen atmosphere. Desired product could be detected by LCMS. The resulting mixture was extracted with CH₂Cl₂:MeOH (3×10 mL). The combined organic layers were dried over anhydrous Na₂SO₄. After filtration, the filtrate was concentrated under reduced pressure. The resulting mixture was concentrated under vacuum and dissolved in DMSO. The crude product (100.00 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 20% B to 45% B in 9 min, 45% B; Wave Length: 254/220 nm; RT1(min): 8.85; Number Of Runs: 0) to afford 3-[(3-fluoro-2-methoxyphenyl)amino]-2-(3-{[(2S)-2-methyl-1-(prop-2-enoyl)pyrrolidin-2-yl]methoxy}pyridin-4-yl)-1H,5H,6H,7H-pyrrolo[3,2-c]pyridin-4-one (9.70 mg, 14.48%) as a yellow solid.

LC-MS: (M+H)⁺ found 520.1.

¹H NMR (400 MHz, Chloroform-d) δ 11.71 (s, 1H), 8.24 (s, 1H), 7.95 (d, J=5.5 Hz, 1H), 7.71 (s, 1H), 7.47 (d, J=5.5 Hz, 1H), 6.68-6.41 (m, 3H), 6.35 (m, J=16.6, 2.1 Hz, 1H), 6.07 (m, J=8.2, 1.4 Hz, 1H), 5.73 (m, J=10.2, 2.1 Hz, 1H), 5.22 (s, 1H), 4.41-4.20 (m, 2H), 4.11 (d, J=1.3 Hz, 3H), 3.91-3.68 (m, 2H), 3.68-3.50 (m, 2H), 3.12 (m, J=7.6, 6.0 Hz, 2H), 2.16-2.02 (m, 3H), 2.00-1.95 (m, 1H), 1.72 (s, 3H).

### Example 203. 2-(3-{[(2R)-1-(but-2-ynoyl)azetidin-2-yl]methoxy}pyridin-4-yl)-3-[(2-ethoxy-3-fluorophenyl)amino]-1H,5H,6H,7H-pyrrolo[3,2-c]pyridin-4-one (compound 352)

### 203.1. Synthesis of tert-butyl (2R)-2-{[(4-{3-[(2-ethoxy-3-fluorophenyl)amino]-4-oxo-1H,5H,6H,7H-pyrrolo[3,2-c]pyridin-2-yl}pyridin-3-yl)oxy]methyl}azetidine-1-carboxylate

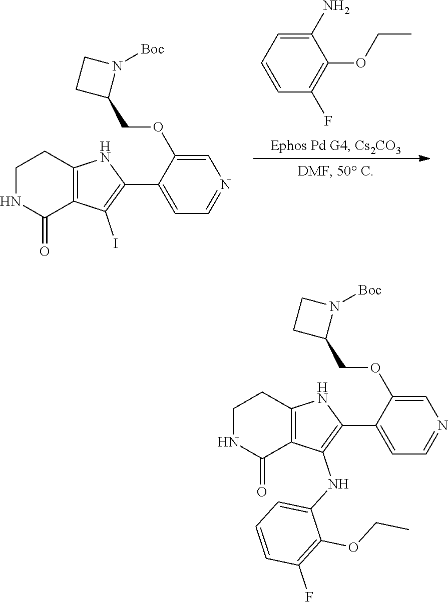

To a stirred mixture of tert-butyl (2R)-2-{[(4-{3-iodo-4-oxo-1H,5H,6H,7H-pyrrolo[3,2-c]pyridin-2-yl}pyridin-3-yl)oxy]methyl}azetidine-1-carboxylate (20.00 mg, 0.04 mmol, 1.00 equiv.) and 2-ethoxy-3-fluoroaniline (9.00 mg, 0.06 mmol, 1.50 equiv.) in DMF (0.3 mL) were added Ephos Pd G4 (4.00 mg, 0.01 mmol, 0.10 equiv.) and Cs₂CO₃ (25.00 mg, 0.08 mmol, 2.00 equiv.) under Argon atmosphere. The resulting suspension was backfilled with Argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl (2R)-2-{[(4-{3-[(2-ethoxy-3-fluorophenyl)amino]-4-oxo-1H,5H,6H,7H-pyrrolo[3,2-c]pyridin-2-yl}pyridin-3-yl)oxy]methyl}azetidine-1-carboxylate (200.00 mg, 950.58%) as a yellow solid.

LC-MS: (M+H)⁺ found 552.2.

### 203.2. Synthesis of 2-{3-[(2R)-azetidin-2-ylmethoxy]pyridin-4-yl}-3-[(2-ethoxy-3-fluorophenyl)amino]-1H,5H,6H,7H-pyrrolo[3,2-c]pyridin-4-one

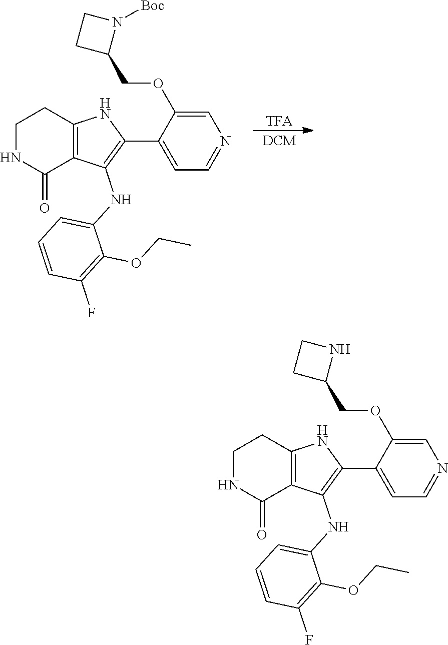

To a stirred mixture of tert-butyl (2R)-2-{[(4-{3-[(2-ethoxy-3-fluorophenyl)amino]-4-oxo-1H,5H,6H,7H-pyrrolo[3,2-c]pyridin-2-yl}pyridin-3-yl)oxy]methyl}azetidine-1-carboxylate (80.00 mg, 0.15 mmol, 1.00 equiv.) in DCM (2 mL) was added TFA (1 mL) at room temperature under nitrogen atmosphere. The resulting mixture was stirred for 1 h and concentrated under reduced pressure to afford 2-{3-[(2R)-azetidin-2-ylmethoxy]pyridin-4-yl}-3-[(2-ethoxy-3-fluorophenyl)amino]-1H,5H,6H,7H-pyrrolo[3,2-c]pyridin-4-one (160.00 mg, crude) as a red oil.

LC-MS: (M+H)⁺ found 452.1.

### 203.3. Synthesis of 2-(3-{[(2R)-1-(but-2-ynoyl)azetidin-2-yl]methoxy}pyridin-4-yl)-3-[(2-ethoxy-3-fluorophenyl)amino]-1H,5H,6H,7H-pyrrolo[3,2-c]pyridin-4-one

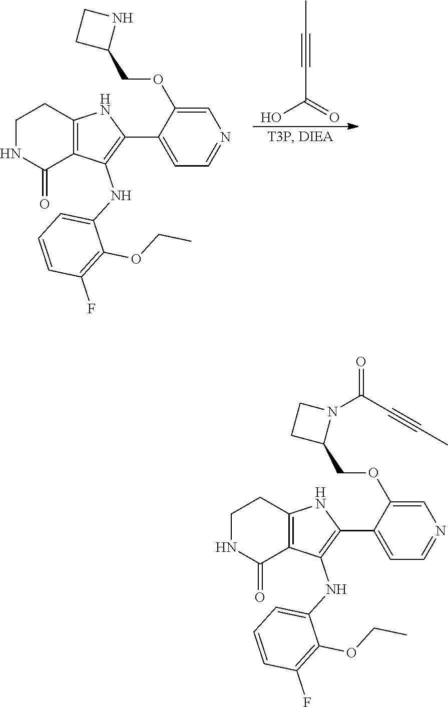

A solution of 2-{3-[(2R)-azetidin-2-ylmethoxy]pyridin-4-yl}-3-[(2-ethoxy-3-fluorophenyl)amino]-1H,5H,6H,7H-pyrrolo[3,2-c]pyridin-4-one (65.00 mg, 0.14 mmol, 1.00 equiv.) in THF (4 mL) was basified to pH 8 with DIEA. To the above mixture was added 2-butynoic acid (24.00 mg, 0.29 mmol, 2.00 equiv.) at 0° C. under nitrogen atmosphere followed by the addition of T₃P (69.00 mg, 0.22 mmol, 1.50 equiv.) dropwise. The resulting mixture was stirred for 1 h at room temperature under nitrogen atmosphere. Saturated aqueous NaHCO₃(10 mL) was added to the reaction mixture at 0° C. and extracted with EtOAc (3×10 mL), dried over anhydrous Na₂SO₄ and filtered. The filtrate was concentrated under reduced pressure to give crude product (60 mg) that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 31% B to 61% B in 9 min, 61% B; Wave Length: 254/220 nm; RT1(min): 8.85; Number Of Runs: 0) to afford 2-(3-{[(2R)-1-(but-2-ynoyl)azetidin-2-yl]methoxy}pyridin-4-yl)-3-[(2-ethoxy-3-fluorophenyl)amino]-1H,5H,6H,7H-pyrrolo[3,2-c]pyridin-4-one (24.70 mg, 33.02%) as a white solid.

LC-MS: (M+H)⁺ found 518.1.

¹H NMR (300 MHz, Chloroform-d) δ 11.42 (s, 1H), 8.25 (s, 1H), 7.94 (d, J=5.4 Hz, 1H), 7.73 (s, 1H), 7.43 (d, J=5.4 Hz, 1H), 6.62-6.40 (m, 2H), 6.05 (d, J=8.1 Hz, 1H), 5.27 (s, 1H), 5.05-4.87 (m, 1H), 4.50 (t, J=9.8 Hz, 1H), 4.40-4.18 (m, 5H), 3.64-3.53 (m, 2H), 3.25-3.00 (m, 2H), 2.62 (d, J=2.3 Hz, 1H), 2.12 (d, J=7.0 Hz, 1H), 2.04 (t, J=7.0 Hz, 3H), 1.63-1.47 (m, 3H).

### Example 204. 3-[(3-fluoro-2-methoxyphenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one (compound 361)

### 204.1. Synthesis of 2-{3-[(2R)-azetidin-2-ylmethoxy]pyridin-4-yl}-3-[(3-fluoro-2-methoxyphenyl)amino]-1H,5H,6H,7H-pyrrolo[3,2-c]pyridin-4-one

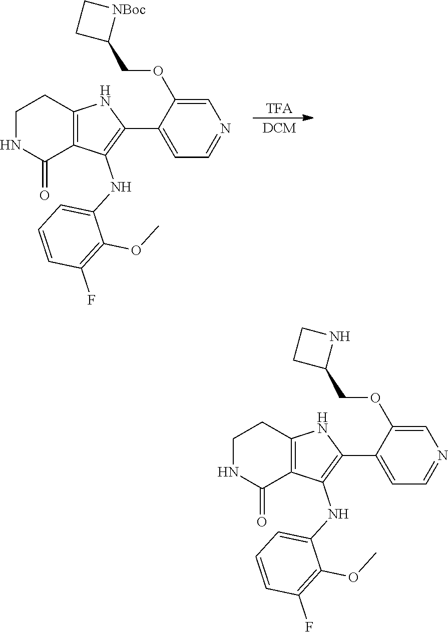

To a stirred mixture of tert-butyl (2R)-2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}azetidine-1-carboxylate (100.00 mg, 0.19 mmol, 1.00 equiv.) in DCM (2 mL) was added TFA (1 mL) at room temperature under nitrogen atmosphere. The resulting mixture was stirred for 1 h and concentrated under reduced pressure to afford 2-{3-[(2R)-azetidin-2-ylmethoxy]pyridin-4-yl}-3-[(3-fluoro-2-methoxyphenyl)amino]-1H,5H,6H,7H-pyrrolo[3,2-c]pyridin-4-one (247.00 mg, crude) as a red oil.

LC-MS: (M+H)⁺ found 438.2.

### 204.2. Synthesis of 3-[(3-fluoro-2-methoxyphenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one

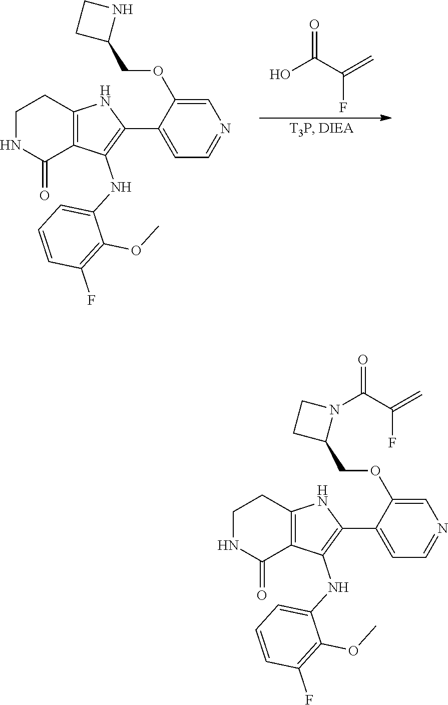

A solution of 2-{3-[(2R)-azetidin-2-ylmethoxy]pyridin-4-yl}-3-[(3-fluoro-2-methoxyphenyl)amino]-1H,5H,6H,7H-pyrrolo[3,2-c]pyridin-4-one (81.00 mg, 0.19 mmol, 1.00 equiv.) in THF (4 mL) was basified to pH 8 with DIEA. To the above mixture was added 2-fluoroprop-2-enoic acid (33.00 mg, 0.37 mmol, 2.00 equiv.) at 0° C. under nitrogen atmosphere followed by the addition of T₃P (88.00 mg, 0.28 mmol, 1.50 equiv.) dropwise. The resulting mixture was stirred for 1 h at room temperature under nitrogen atmosphere. Saturated aqueous NaHCO₃(10 mL) was added to the reaction mixture at 0° C. and extracted with EtOAc (3×10 mL), dried over anhydrous Na₂SO₄ and filtered. The filtrate was concentrated under reduced pressure to give crude product (60.00 mg) that was purified by Prep-HPLC under the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 26% B to 52% B in 8 min, 52% B; Wave Length: 254/220 nm; RT1(min): 6.32; Number Of Runs: 0) to afford 3-[(3-fluoro-2-methoxyphenyl)amino]-2-(3-{[(2R)-1-(2-fluoroprop-2-enoyl)azetidin-2-yl]methoxy}pyridin-4-yl)-1H,5H,6H,7H-pyrrolo[3,2-c]pyridin-4-one (19.20 mg, 20.23%) as a white solid.

LC-MS: (M+H)⁺ found 510.2.

¹H NMR (400 MHz, Chloroform-d) δ 11.59 (s, 1H), 8.26 (s, 1H), 7.95 (d, J=5.6 Hz, 1H), 7.79 (s, 1H), 7.49 (d, J=5.6 Hz, 1H), 6.69-6.54 (m, 1H), 6.52 (d, J=9.4 Hz, 1H), 6.04 (d, J=8.1 Hz, 1H), 5.62 (dd, J=45.7, 3.2 Hz, 1H), 5.28-5.15 (m, 2H), 5.10 (q, J=8.7 Hz, 1H), 4.66-4.38 (m, 3H), 4.32 (d, J=9.6 Hz, 1H), 4.11 (s, 3H), 3.70-3.52 (m, 2H), 3.17-3.08 (m, 2H), 2.77-2.63 (m, 1H), 2.23-2.14 (m, 1H).

### Example 205. 3-[(3-chloro-2-methoxyphenyl)amino]-2-(3-{[(1S,3R,4R)-2-(prop-2-enoyl)-2-azabicyclo[2.2.1]heptan-3-yl]methoxy}pyridin-4-yl)-1H,5H,6H,7H-pyrrolo[3,2-c]pyridin-4-one (compound 274)

### 205.1. Synthesis of tert-butyl (1S,3R,4R)-3-(hydroxymethyl)-2-azabicyclo[2.2.1]heptane-2-carboxylate

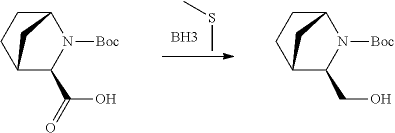

To a stirred solution of (1S,3R,4R)-2-(tert-butoxycarbonyl)-2-azabicyclo[2.2.1]heptane-3-carboxylic acid (5 g, 20.72 mmol, 1 equiv) in THF (50 mL) were added BH₃-THF (2.15 g, 24.865 mmol, 1.2 equiv) dropwise at 0° C. under N₂ atmosphere. The resulting mixture was stirred for 2h at RT under N₂ atmosphere. The reaction was monitored by TLC. The reaction was quenched by the addition of saturated aqueous NaHCO₃ at 0° C. The aqueous layer was extracted with EA 500 ml. The resulting mixture was washed with saturated aqueous NaCl 500 ml. The resulting mixture was dried Na₂SO₄. The resulting mixture was concentrated under reduced pressure. This resulted in tert-butyl (1S,3R,4R)-3-(hydroxymethyl)-2-azabicyclo[2.2.1]heptane-2-carboxylate (3.5 g, 99.78%) as a colourless oil.

LC-MS: M+H found: 228.

### 205.2. Synthesis of tert-butyl (1S,3R,4R)-3-{[(4-chloropyridin-3-yl)oxy]methyl}-2-azabicyclo[2.2.1]heptane-2-carboxylate

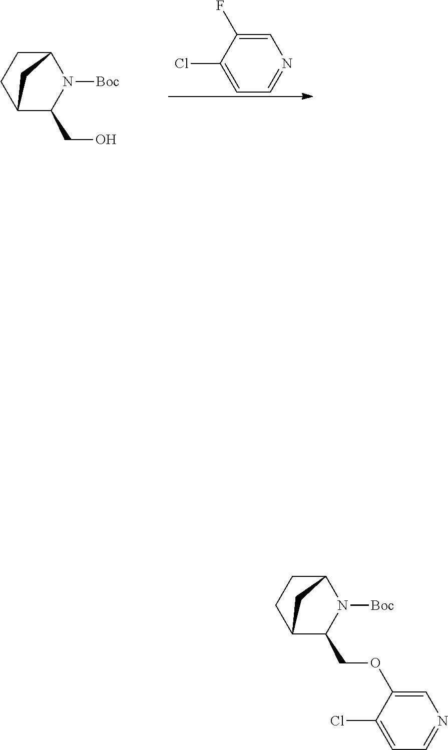 [SMILES: CC(C)(C)OC(=O)N1[C@H]2CC[C@H](C2)[C@@H]1CO ; Fc1cnccc1Cl ; CC(C)(C)OC(=O)N1[C@H]2CC[C@H](C2)[C@@H]1COc1cnccc1Cl]

A mixture of 4-chloro-3-fluoropyridine (1300 mg, 10.559 mmol, 1 equiv) and NaH (760.14 mg, 31.677 mmol, 3 equiv) in DMF (20 mL) was stirred for 20 min at 0° C. under nitrogen atmosphere. To the above mixture was added 4-chloro-3-fluoropyridine (1388.77 mg, 10.559 mmol, 1 equiv) in portions. The resulting mixture was stirred for additional overnight at room temperature. The reaction was quenched with Water at 0° C. The resulting mixture was extracted with EtOAc (3×50 mL). The combined organic layers were washed with brine (1×200 mL), dried over anhydrous Na₂SO₄. After filtration, the filtrate was concentrated under reduced pressure. The residue was purified by silica gel column chromatography, eluted with PE/EA (2:1) to afford tert-butyl (1S,3R,4R)-3-{[(4-chloropyridin-3-yl)oxy]methyl}-2-azabicyclo[2.2.1]heptane-2-carboxylate (1000 mg, 40.25%) as a colorless solid.

LC-MS: M+H found: 339.

### 205.3. Synthesis of tert-butyl (1S,3R,4R)-3-{[(4-{4-oxo-1H,5H,6H,7H-pyrrolo[3,2-c]pyridin-2-yl}pyridin-3-yl)oxy]methyl}-2-azabicyclo[2.2.1]heptane-2-carboxylate

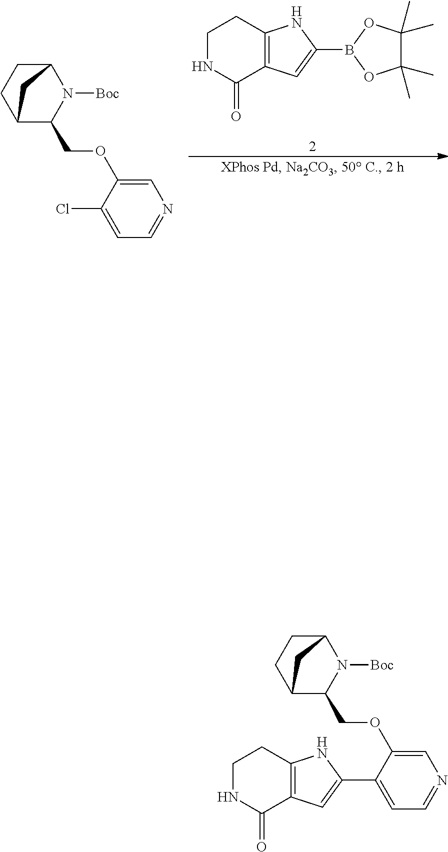 [SMILES: CC(C)(C)OC(=O)N1[C@H]2CC[C@H](C2)[C@@H]1COc1cnccc1Cl ; CC1(C)OB(c2cc3c([nH]2)CCNC3=O)OC1(C)C ; "XPhos Pd, Na2CO3, 50° C., 2 h" ; CC(C)(C)OC(=O)N1[C@H]2CC[C@H](C2)[C@@H]1COc1cnccc1-c1cc2c([nH]1)CCNC2=O]

To a stirred solution of tert-butyl (1S,3R,4R)-3-{[(4-chloropyridin-3-yl)oxy]methyl}-2-azabicyclo[2.2.1]heptane-2-carboxylate (1000 mg, 0.0295 mmol, 1 equiv), 2-(4,4,5,5-tetramethyl-1,3,2-dioxaborolan-2-yl)-1H,5H,6H,7H-pyrrolo[3,2-c]pyridin-4-one (696.245 mg, 2.656 mmol, 1 equiv) and Na₂CO₃ (563.055 mg, 5.312 mmol, 2 equiv) in THF (10 mL) and H₂O (5 mL) was added XPhos Pd G3 (250.00 mg, 0.003 mmol, 0.1 equiv) dropwise at 50° C. under nitrogen atmosphere. The resulting mixture was extracted with EtOAc (3×50 mL). The combined organic layers were washed with brine (2×100 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl (1S,3R,4R)-3-{[(4-{4-oxo-1H,5H,6H,7H-pyrrolo[3,2-c]pyridin-2-yl}pyridin-3-yl)oxy]methyl}-2-azabicyclo[2.2.1]heptane-2-carboxylate (1000 mg, 85.85%) as a colorless solid.

LC-MS: M+H found: 439.

### 205.4. Synthesis of (1S,3R,4R)-tert-butyl 3-((4-(3-iodo-4-oxo-4,5,6,7-tetrahydro-1H-pyrrolo[3,2-c]pyridin-2-yl)pyridin-3-yloxy)methyl)-2-aza-bicyclo[2.2.1]heptane-2-carboxylate

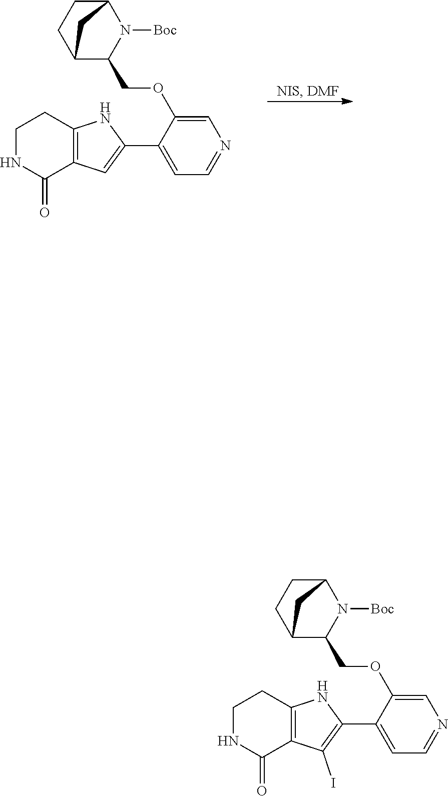

A solution of tert-butyl (1S,3R,4R)-3-{[(4-{4-oxo-1H,5H,6H,7H-pyrrolo[3,2-c]pyridin-2-yl}pyridin-3-yl)oxy]methyl}-2-azabicyclo[2.2.1]heptane-2-carboxylate (900 mg, 0.38 mmol, 1 equiv) and NIS (102.6 mg, 0.457 mmol, 1.2 equiv) in DMF (10 mL, 8.615 mmol, 226.66 equiv) was stirred for h at 50° C. under nitrogen atmosphere. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with CH₂Cl₂/MeOH (10:1) to afford (1S,3R,4R)-tert-butyl 3-((4-(3-iodo-4-oxo-4,5,6,7-tetrahydro-1H-pyrrolo[3,2-c]pyridin-2-yl)pyridin-3-yloxy)methyl)-2-aza-bicyclo[2.2.1]heptane-2-carboxylate (800 mg, 80.85%) as a colorless solid.

LC-MS: M+H found: 565.

### 205.5. Synthesis of tert-butyl (1S,3R,4R)-3-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-2-azabicyclo[2.2.1]heptane-2-carboxylate

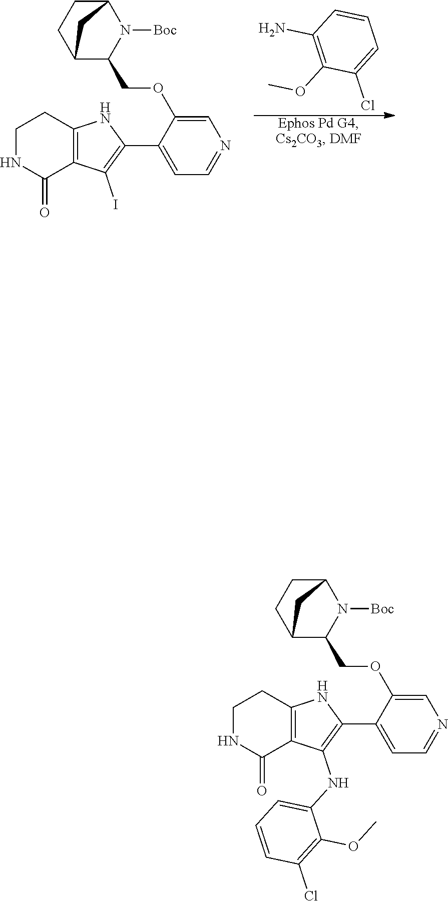

A mixture of tert-butyl (1S,3R,4R)-3-{[(4-{3-iodo-4-oxo-1H,5H,6H,7H-pyrrolo[3,2-c]pyridin-2-yl}pyridin-3-yl)oxy]methyl}-2-azabicyclo[2.2.1]heptane-2-carboxylate (200 mg, 0.354 mmol, 1 equiv), 3-fluoro-2-methoxyaniline (75.02 mg, 0.531 mmol, 1.5 equiv), EPhos Pd G4 (32.55 mg, 0.035 mmol, 0.1 equiv), EPhos (37.90 mg, 0.071 mmol, 0.2 equiv) and Cs₂CO₃ (346.36 mg, 1.062 mmol, 3 equiv) in DMF (4 mL) was stirred for 2 h at 50° C. under nitrogen atmosphere. The resulting mixture was diluted with water. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with CH₂Cl₂/MeOH (20:1) to afford tert-butyl (1S,3R,4R)-3-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-2-azabicyclo[2.2.1]heptane-2-carboxylate (100 mg, 48.85%) as a yellow solid.

LC-MS: M+H found: 594.

### 205.6. Synthesis of 2-{3-[(1S,3R,4R)-2-azabicyclo[2.2.1]heptan-3-ylmethoxy]pyridin-4-yl}-3-[(3-chloro-2-methoxyphenyl)amino]-1H,5H,6H,7H-pyrrolo[3,2-c]pyridin-4-one

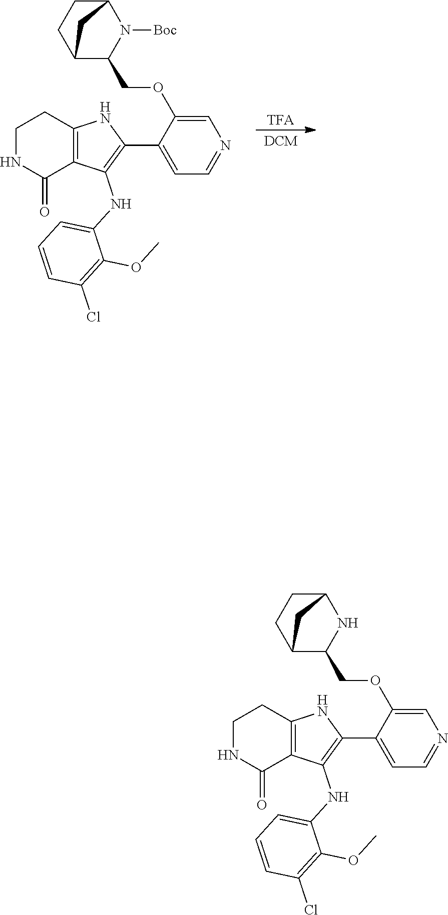

A solution of tert-butyl (1S,3R,4R)-3-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-2-azabicyclo[2.2.1]heptane-2-carboxylate (100 mg, 0.16.8 mmol, 1 equiv) and TFA (191.923 mg, 1.6830 mmol, 10 equiv) in DCM (5 mL) was stirred for 2 h at room temperature under nitrogen atmosphere. The reaction was quenched with IPA at room temperature. The resulting mixture was concentrated under vacuum. The residue was purified by silica gel column chromatography, eluted with CH₂Cl₂/MeOH (10:1) to afford 2-{3-[(1S,3R,4R)-2-azabicyclo[2.2.1]heptan-3-ylmethoxy]pyridin-4-yl}-3-[(3-chloro-2-methoxyphenyl)amino]-1H,5H,6H,7H-pyrrolo[3,2-c]pyridin-4-one (70 mg, 84.19%) as a brown solid.

LC-MS: M+H found: 494.

### 205.7. Synthesis of 3-[(3-chloro-2-methoxyphenyl)amino]-2-(3-{[(1S,3R,4R)-2-(prop-2-enoyl)-2-azabicyclo[2.2.1]heptan-3-yl]methoxy}pyridin-4-yl)-1H,5H,6H,7H-pyrrolo[3,2-c]pyridin-4-one

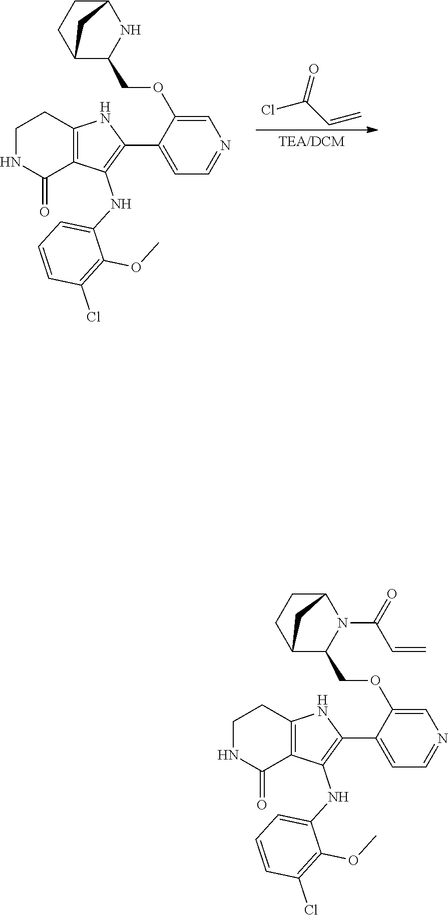

To a stirred solution of 2-{3-[(1S,3R,4R)-2-azabicyclo[2.2.1]heptan-3-ylmethoxy]pyridin-4-yl}-3-[(3-chloro-2-methoxyphenyl)amino]-1H,5H,6H,7H-pyrrolo[3,2-c]pyridin-4-one (60 mg, 0.121 mmol, 1.0 equiv) and TEA (24.58 mg, 0.243 mmol, 2.0 equiv) in CH₂Cl₂ (5 mL) were added acryloyl chloride (10.98 mg, 0.121 mmol, 1.0 equiv) dropwise at 0° C. The resulting mixture was stirred for 1 h at room temperature. After the reaction was completed, the mixture was diluted with water, extracted with EA, washed with brine, dried over anhydrous Na₂SO₄ to the crude product. The residue was purified by Prep-TLC (Column: Sunfire prep C18 column, 30*150 mm, 5 μm; Mobile Phase A: Water (0.1% FA), Mobile Phase B: ACN; Flow rate: 60 mL/min; Gradient: 15% B to 45% B in 7 min, 45% B; Wave Length: 254/220 nm; RT1(min): 6.9; Number Of Runs: 0) to afford 3-[(3-chloro-2-methoxyphenyl)amino]-2-(3-{[(1S,3R,4R)-2-(prop-2-enoyl)-2-azabicyclo[2.2.1]heptan-3-yl]methoxy}pyridin-4-yl)-1H,5H,6H,7H-pyrrolo[3,2-c]pyridin-4-one (48.4 mg, 70.634%) as a yellow solid.

LC-MS: M+H found: 494.

¹H NMR (300 MHz, DMSO-d6) δ 11.73 (s, 1H), 8.46 (s, 1H), 8.00 (d, J=5.0 Hz, 1H), 7.54 (s, 1H), 7.34 (d, J=5.1 Hz, 1H), 7.13 (s, 1H), 6.84-6.57 (m, 3H), 6.26 (dd, J=16.6, 2.3 Hz, 1H), 6.14 (q, J=4.4 Hz, 1H), 5.75 (dd, J=10.3, 2.3 Hz, 1H), 4.61 (s, 1H), 4.34 (dd, J=10.2, 6.6 Hz, 1H), 4.18 (dd, J=10.2, 4.6 Hz, 1H), 4.05 (d, J=5.6 Hz, 1H), 3.89 (s, 3H), 3.34 (s, 2H), 2.96 (t, J=6.8 Hz, 2H), 2.57 (s, 1H), 2.00 (d, J=10.4 Hz, 1H), 1.72 (d, J=8.1 Hz, 2H), 1.44 (t, J=9.4 Hz, 3H).

### Example 206. 3-[(3-chloro-2-methoxyphenyl)amino]-2-(3-{[(2S)-3,3-dimethyl-1-(prop-2-enoyl)azetidin-2-yl]methoxy}pyridin-4-yl)-1H,5H,6H,7H-pyrrolo[3,2-c]pyridin-4-one (compound 512)

### 206.1. Synthesis of tert-butyl 2-{[(4-chloropyridin-3-yl)oxy]methyl}-3,3-dimethylazetidine-1-carboxylate

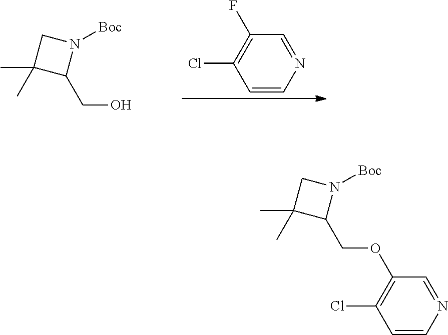 [SMILES: CC(C)(C)OC(=O)N1CC(C)(C)C1CO ; Fc1cnccc1Cl ; CC(C)(C)OC(=O)N1CC(C)(C)C1COc1cnccc1Cl]

To a solution of tert-butyl (2S)-2-(hydroxymethyl)pyrrolidine-1-carboxylate (2 g, 9.937 mmol, 1 equiv) in THF was added sodium hydride (60% in oil, NaH (0.60 g, 14.905 mmol, 1.5 equiv, 60%)) at 0 degrees C. The mixture was stirred for 15 min. 4-chloro-3-fluoropyridine (1.31 g, 9.937 mmol, 1 equiv) was added and the mixture was allowed to warm to RT and stirred for 20 hours. The reaction mixture was quenched by water and extracted with DCM (3*25 mL). To afford tert-butyl 2-{[(4-chloropyridin-3-yl)oxy]methyl}-3,3-dimethylazetidine-1-carboxylate (1268 mg, 41.76%) as a yellow oil.

LC-MS: M+H found: 327.35.

### 206.2. Synthesis of tert-butyl 3,3-dimethyl-2-{[(4-{4-oxo-1H,5H,6H,7H-pyrrolo[3,2-c]pyridin-2-yl}pyridin-3-yl)oxy]methyl}azetidine-1-carboxylate

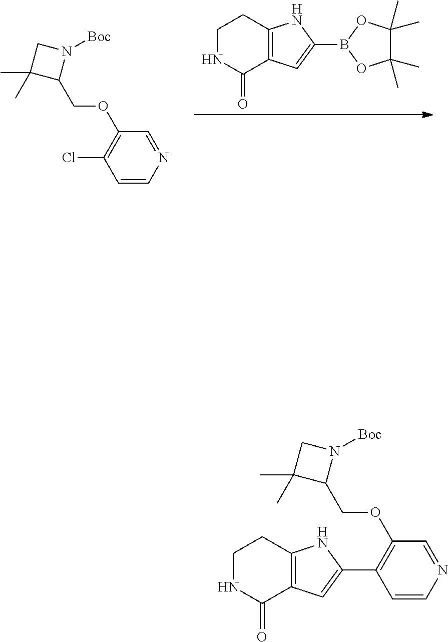 [SMILES: CC(C)(C)OC(=O)N1CC(C)(C)C1COc1cnccc1Cl ; CC1(C)OB(c2cc3c([nH]2)CCNC3=O)OC1(C)C ; CC(C)(C)OC(=O)N1CC(C)(C)C1COc1cnccc1-c1cc2c([nH]1)CCNC2=O]

To a solution of tert-butyl 2-{[(4-chloropyridin-3-yl)oxy]methyl}-3,3-dimethylazetidine-1-carboxylate (1168 mg, 3.574 mmol, 1 equiv) and 2-(4,4,5,5-tetramethyl-1,3,2-dioxaborolan-2-yl)-1H,5H,6H,7H-pyrrolo[3,2-c]pyridin-4-one (1405.16 mg, 5.361 mmol, 1.5 equiv) in THF (12 mL) and H₂O (3 mL) were added Na₂CO₃ (757.57 mg, 7.148 mmol, 2 equiv) and XPhos Pd G3 (302.51 mg, 0.357 mmol, 0.1 equiv). After stirring for 2 hours at 50° C. under a nitrogen atmosphere, the resulting mixture was concentrated under reduced pressure. The residue was purified by silica gel column chromatography, eluted with DCM and MeOH (92:8) to afford tert-butyl 3,3-dimethyl-2-{[(4-{4-oxo-1H,5H,6H,7H-pyrrolo[3,2-c]pyridin-2-yl}pyridin-3-yl)oxy]methyl}azetidine-1-carboxylate (1200 mg, 78.72%) as a yellow solid.

LC-MS: M+H found: 427.15.

### 206.3. Synthesis of tert-butyl 2-{[(4-{3-iodo-4-oxo-1H,5H,6H,7H-pyrrolo[3,2-c]pyridin-2-yl}pyridin-3-yl)oxy]methyl}-3,3-dimethylazetidine-1-carboxylate

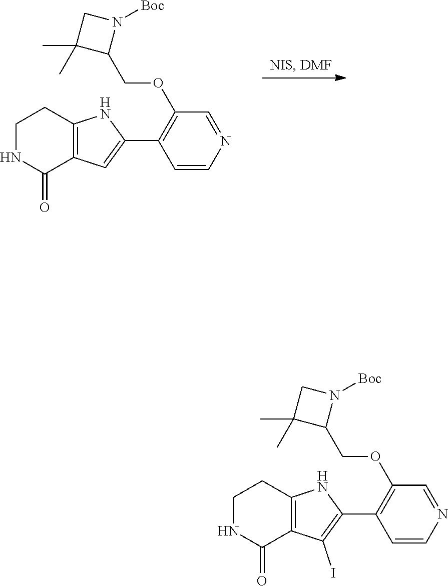 [SMILES: CC(C)(C)OC(=O)N1CC(C)(C)C1COc1cnccc1-c1cc2c([nH]1)CCNC2=O ; CC(C)(C)OC(=O)N1CC(C)(C)C1COc1cnccc1-c1[nH]c2c(c1I)C(=O)NCC2]

To a stirred solution of tert-butyl 3,3-dimethyl-2-{[(4-{4-oxo-1H,5H,6H,7H-pyrrolo[3,2-c]pyridin-2-yl}pyridin-3-yl)oxy]methyl}azetidine-1-carboxylate (1200 mg, 2.813 mmol, 1 equiv) in DMF (12 mL) was added NIS (949.49 mg, 4.220 mmol, 1.5 equiv) in portions at RT. The resulting mixture was stirred for 1 hour at RT. The resulting mixture was extracted with EA (3×50 mL). The combined organic layer was concentrated under reduced pressure. The residue was purified by Prep-TLC (DCM and MeOH 15:1) to afford tert-butyl 2-{[(4-{3-iodo-4-oxo-1H,5H,6H,7H-pyrrolo[3,2-c]pyridin-2-yl}pyridin-3-yl)oxy]methyl}-3,3-dimethylazetidine-1-carboxylate (1450 mg, 93.30%) as a yellow solid.

LC-MS: M+H found: 553.1.

### 206.4. Synthesis of tert-butyl 2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-3,3-dimethylazetidine-1-carboxylate

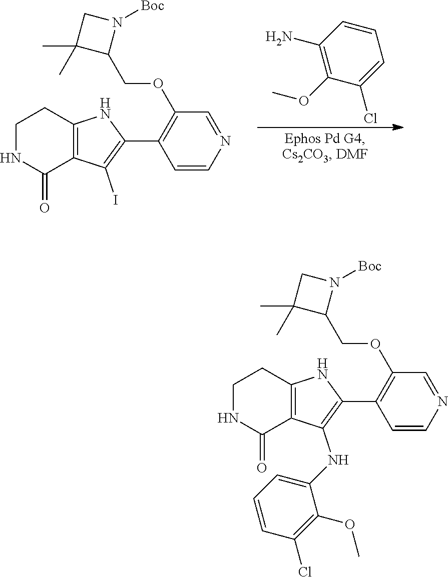

To a stirred mixture of tert-butyl 2-{[(4-{3-iodo-4-oxo-1H,5H,6H,7H-pyrrolo[3,2-c]pyridin-2-yl}pyridin-3-yl)oxy]methyl}-3,3-dimethylazetidine-1-carboxylate (1400 mg, 2.534 mmol, 1 equiv) and 3-chloro-2-methoxyaniline (1198.23 mg, 7.602 mmol, 3 equiv) in DMF (15 mL) were added Cs₂CO₃ (2477.21 mg, 7.602 mmol, 3 equiv) and EPhos Pd G4 (232.79 mg, 0.253 mmol, 0.1 equiv) in portions at RT under N₂ atmosphere. The resulting mixture was stirred for 2 hours at 50 degrees C. under N₂ atmosphere. The resulting mixture was filtered, then the filter cake was washed with EA (1×1 100 mL). The filtrate was concentrated under reduced pressure. The residue was purified by Prep-TLC (DCM and MeOH 20:1) to afford tert-butyl 2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-3,3-dimethylazetidine-1-carboxylate (1.4 g, 94.90%) as a light yellow solid.

### 206.5. Synthesis of 3-[(3-chloro-2-methoxyphenyl)amino]-2-{3-[(3,3-dimethylazetidin-2-yl)methoxy]pyridin-4-yl}-1H,5H,6H,7H-pyrrolo[3,2-c]pyridin-4-one

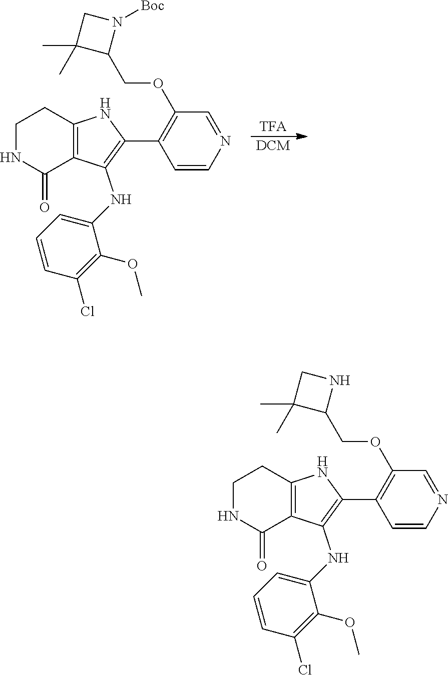

To a stirred solution of tert-butyl 2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-3,3-dimethylazetidine-1-carboxylate (1.5 g, 2.577 mmol, 1 equiv) in DCM (15 mL) was added TFA (7 mL, 94.241 mmol, 36.57 equiv) dropwise at RT. The resulting mixture was stirred for 1.5 hour at RT. The aqueous layer was extracted with DCM and H₂O (3×1 150 mL). To afford 3-[(3-chloro-2-methoxyphenyl)amino]-2-{3-[(3,3-dimethylazetidin-2-yl)methoxy]pyridin-4-yl}-1H,5H,6H,7H-pyrrolo[3,2-c]pyridin-4-one (1.1 g, 88.57%) as a light yellow solid.

LC-MS: M+H found: 482.45.

### 206.6. Synthesis of 3-[(3-chloro-2-methoxyphenyl)amino]-2-(3-{[3,3-dimethyl-1-(prop-2-enoyl)azetidin-2-yl]methoxy}pyridin-4-yl)-1H,5H,6H,7H-pyrrolo[3,2-c]pyridin-4-one

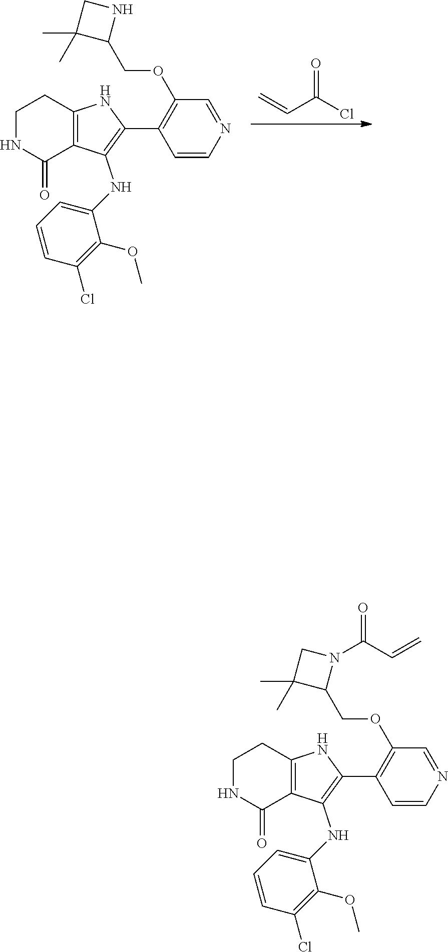

To a mixture of 3-[(3-chloro-2-methoxyphenyl)amino]-2-{3-[(3,3-dimethylazetidin-2-yl)methoxy]pyridin-4-yl}-1H,5H,6H,7H-pyrrolo[3,2-c]pyridin-4-one (700 mg, 1.452 mmol, 1 equiv) in THF (10 mL) and a.q. NaHCO₃(366.02 mg, 4.356 mmol, 3 equiv) was added acryloyl chloride (131.45 mg, 1.452 mmol, 1 equiv) dropwise at 0 degrees C. The mixture was stirred at 0 degrees C. for 1 h. Desired product could be detected by LCMS. The resulting mixture was extracted with EA (3×300 ml). After filtration, the filtrate was concentrated under reduced pressure. The residue was purified by Prep-TLC (PE:EA=10:1) to afford the crude product. The crude product was purified by Prep-HPLC to afford 3-[(3-chloro-2-methoxyphenyl)amino]-2-(3-{[3,3-dimethyl-1-(prop-2-enoyl)azetidin-2-yl]methoxy}pyridin-4-yl)-1H,5H,6H,7H-pyrrolo[3,2-c]pyridin-4-one (404 mg, 51.89%) as a yellow solid.

LC-MS: M+H found: 536.15.

### 206.7. Synthesis of 3-[(3-chloro-2-methoxyphenyl)amino]-2-(3-{[(2S)-3,3-dimethyl-1-(prop-2-enoyl)azetidin-2-yl]methoxy}pyridin-4-yl)-1H,5H,6H,7H-pyrrolo[3,2-c]pyridin-4-one

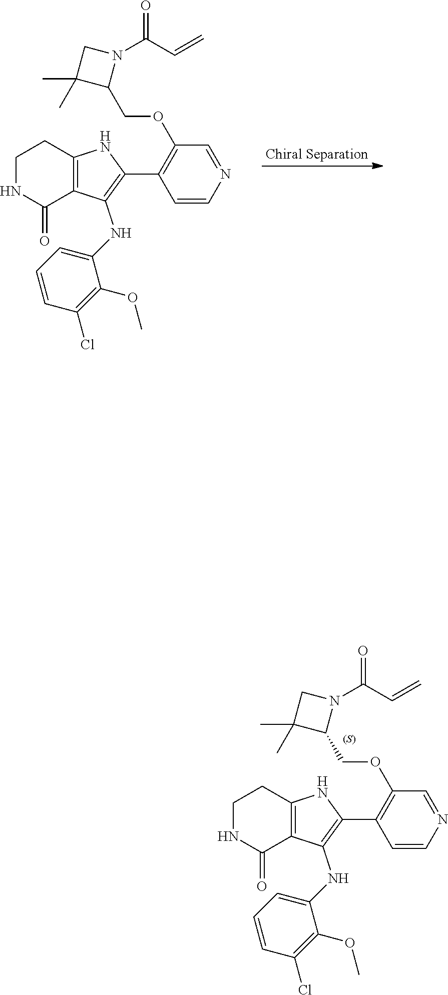

The crude product 3-[(3-chloro-2-methoxyphenyl)amino]-2-(3-{[3,3-dimethyl-1-(prop-2-enoyl)azetidin-2-yl]methoxy}pyridin-4-yl)-1H,5H,6H,7H-pyrrolo[3,2-c]pyridin-4-one (170 mg, 0.317 mmol, 1 equiv) was purified by Prep-Chiral—HPLC with the following conditions (Column: CHIRAL ART Amylose-SA, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 50% B to 50% B in 18 min; Wave Length: 220/254 nm; RT1(min): 6.51; RT2(min): 14.92; Sample Solvent: EtOH—HPLC; Injection Volume: 3 mL; Number Of Runs: 2) to afford 3-[(3-chloro-2-methoxyphenyl)amino]-2-(3-{[(2S)-3,3-dimethyl-1-(prop-2-enoyl)azetidin-2-yl]methoxy}pyridin-4-yl)-1H,5H,6H,7H-pyrrolo[3,2-c]pyridin-4-one (55.5 mg, 32.48%) as a yellow solid.

LC-MS: (M+H)⁺ found 536.15.

¹H NMR (400 MHz, DMSO-d6) δ 11.72 (s, 1H), 8.39 (s, 1H), 7.96 (d, J=5.1 Hz, 1H), 7.43 (s, 1H), 7.32 (d, J=5.0 Hz, 1H), 6.81 (s, 1H), 6.68-6.57 (m, 2H), 6.35 (dd, J=17.0, 10.2 Hz, 1H), 6.22-6.11 (m, 2H), 5.69 (s, 1H), 4.56 (t, J=9.0 Hz, 1H), 4.45-4.35 (m, 2H), 3.89 (s, 4H), 3.84 (s, 1H), 3.41 (td, J=6.9, 2.4 Hz, 2H), 2.92 (s, 3H), 1.24 (d, J=21.7 Hz, 6H)

### Example 207. 3-[(3-chloro-2-methoxyphenyl)amino]-2-(3-{[(2R)-3,3-dimethyl-1-(prop-2-enoyl)azetidin-2-yl]methoxy}pyridin-4-yl)-1H,5H,6H,7H-pyrrolo[3,2-c]pyridin-4-one (compound 513)

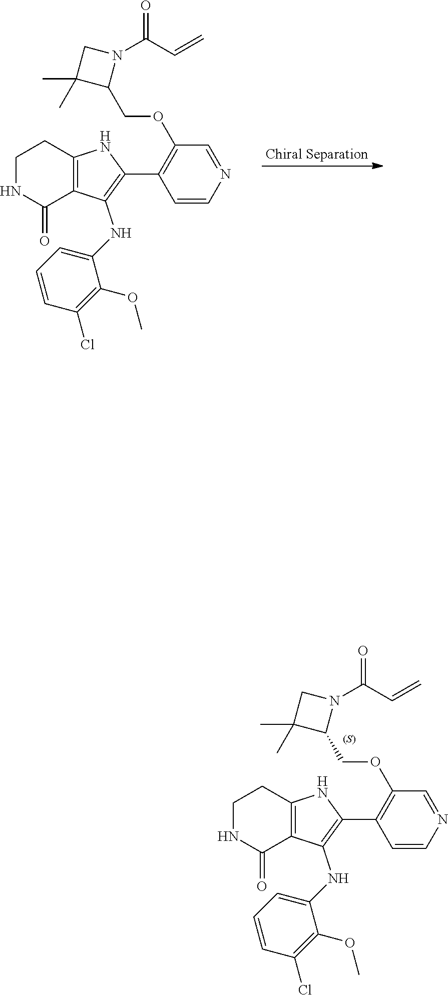

The crude product 3-[(3-chloro-2-methoxyphenyl)amino]-2-(3-{[3,3-dimethyl-1-(prop-2-enoyl)azetidin-2-yl]methoxy}pyridin-4-yl)-1H,5H,6H,7H-pyrrolo[3,2-c]pyridin-4-one (170 mg, 0.317 mmol, 1 equiv) was purified by Prep-Chiral—HPLC with the following conditions (Column: CHIRAL ART Amylose-SA, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 50% B to 50% B in 18 min; Wave Length: 220/254 nm; RT1(min): 6.51; RT2(min): 14.92; Sample Solvent: EtOH—HPLC; Injection Volume: 3 mL; Number Of Runs: 2) to afford 3-[(3-chloro-2-methoxyphenyl)amino]-2-(3-{[(2R)-3,3-dimethyl-1-(prop-2-enoyl)azetidin-2-yl]methoxy}pyridin-4-yl)-1H,5H,6H,7H-pyrrolo[3,2-c]pyridin-4-one (50.5 mg, 29.59%) as a yellow solid.

LC-MS: (M+H)⁺ found 536.15.

¹H NMR (400 MHz, DMSO-d6) δ 11.72 (s, 1H), 8.39 (s, 1H), 7.96 (d, J=5.1 Hz, 1H), 7.43 (s, 1H), 7.32 (d, J=5.0 Hz, 1H), 6.81 (s, 1H), 6.68-6.57 (m, 2H), 6.35 (dd, J=17.0, 10.2 Hz, 1H), 6.22-6.11 (m, 2H), 5.69 (s, 1H), 4.56 (t, J=9.0 Hz, 1H), 4.45-4.35 (m, 2H), 3.89 (s, 4H), 3.84 (s, 1H), 3.41 (td, J=6.9, 2.4 Hz, 2H), 2.92 (s, 3H), 1.24 (d, J=21.7 Hz, 6H).

### Example 208. rel-2-(3-{[(2R)-3,3-dimethyl-1-(prop-2-enoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (compound 356)

### 208.1. Synthesis of tert-butyl 2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-3,3-dimethylazetidine-1-carboxylate

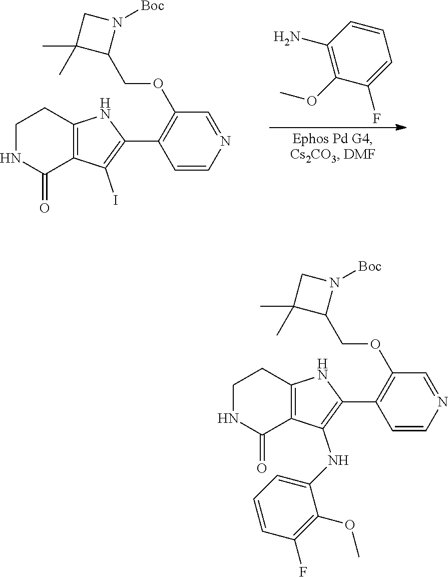

To a stirred mixture of tert-butyl 2-{[(4-{3-iodo-4-oxo-1H,5H,6H,7H-pyrrolo[3,2-c]pyridin-2-yl}pyridin-3-yl)oxy]methyl}-3,3-dimethylazetidine-1-carboxylate (650 mg, 1.177 mmol, 1 equiv) and 3-fluoro-2-methoxyaniline (498.24 mg, 3.531 mmol, 3 equiv) in DMF (6 mL) were added Cs₂CO₃ (1150.13 mg, 3.531 mmol, 3 equiv) and EPhos Pd G4 (108.08 mg, 0.118 mmol, 0.1 equiv) in portions at RT under N₂ atmosphere. The resulting mixture was stirred for 2 hours at 50 degrees C. under N₂ atmosphere. The resulting mixture was filtered, then the filter cake was washed with EA (1×1 30 mL). The filtrate was concentrated under reduced pressure. The residue was purified by Prep-TLC (DCM and MeOH 20:1) to afford tert-butyl 2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-3,3-dimethylazetidine-1-carboxylate (650 mg, 97.66%) as a light yellow solid.

LC-MS: (M+H)⁺ found 566.2.

### 208.2. Synthesis of 2-{3-[(3,3-dimethylazetidin-2-yl)methoxy]pyridin-4-yl}-3-[(3-fluoro-2-methoxyphenyl)amino]-1H,5H,6H,7H-pyrrolo[3,2-c]pyridin-4-one

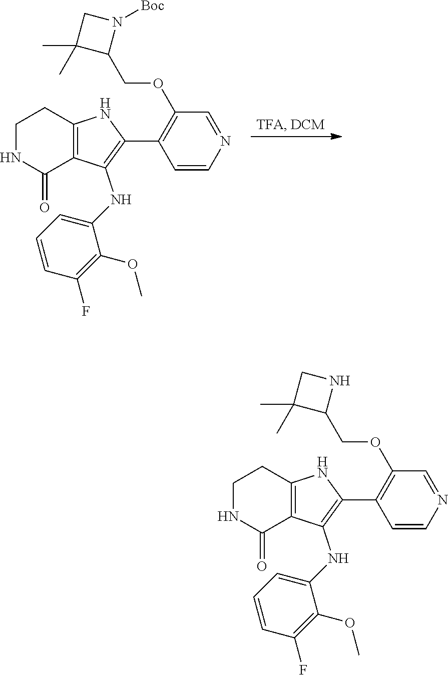

To a stirred solution of tert-butyl 2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-3,3-dimethylazetidine-1-carboxylate (1000 mg, 1.768 mmol, 1 equiv) in DCM (10 mL) was added TFA (5 mL, 67.315 mmol, 38.08 equiv) dropwise at RT. The resulting mixture was stirred for 1.5 hour at RT. The aqueous layer was extracted with DCM and H₂O (3×1 150 mL). To afford 2-{3-[(3,3-dimethylazetidin-2-yl)methoxy]pyridin-4-yl}-3-[(3-fluoro-2-methoxyphenyl)amino]-1H,5H,6H,7H-pyrrolo[3,2-c]pyridin-4-one (540 mg, 65.61%) as a light yellow solid.

LC-MS: (M+H)⁺ found 466.1.

### 208.3. Synthesis of 2-(3-{[(2S)-3,3-dimethyl-1-(prop-2-enoyl)azetidin-2-yl]methoxy}pyridin-4-yl)-3-[(3-fluoro-2-methoxyphenyl)amino]-1H,5H,6H,7H-pyrrolo[3,2-c]pyridin-4-one

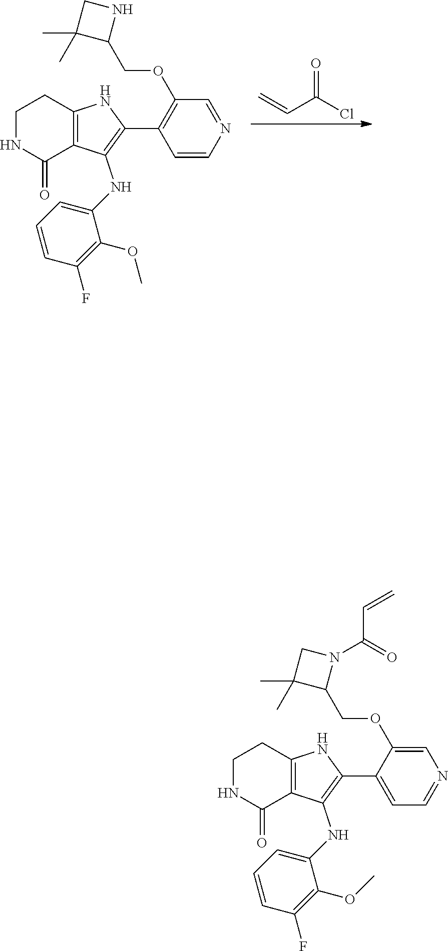

To a stirred solution of 2-{3-[(3,3-dimethylazetidin-2-yl)methoxy]pyridin-4-yl}-3-[(3-fluoro-2-methoxyphenyl)amino]-1H,5H,6H,7H-pyrrolo[3,2-c]pyridin-4-one (100 mg, 0.215 mmol, 1 equiv) and TEA (108.68 mg, 1.075 mmol, 5 equiv) in DCM (2 mL) was added acryloyl chloride (19.44 mg, 0.215 mmol, 1 equiv) dropwise at −30° C. under N₂ atmosphere. The resulting mixture was stirred for 0.5 hour at −30° C. The crude product was purified by Prep-HPLC with the following conditions (Column: YMC-Actus Triart C18, 30*150 mm, 5 μm; Mobile Phase A: Water (10 mmol/L NH₄HCO_{3+0.1}% NH₃·H₂O), Mobile Phase B: ACN; Flow rate: 60 mL/min; Gradient: 45% B to 55% B in 8 min, 55% B; Wave Length: 254; 220 nm; RT1(min): 7.55; Number Of Runs: 0) to afford 2-(3-{[(2S)-3,3-dimethyl-1-(prop-2-enoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (24 mg, 21.50%) as a yellow solid.

LC-MS: (M+H)⁺ found 520.25.

### 208.4. Synthesis of rel-2-(3-{[(2R)-3,3-dimethyl-1-(prop-2-enoyl)azetidin-2-yl]methoxy}pyridin-4-yl)-3-[(3-fluoro-2-methoxyphenyl)amino]-H,5H,6H,7H-pyrrolo[3,2-c]pyridin-4-one

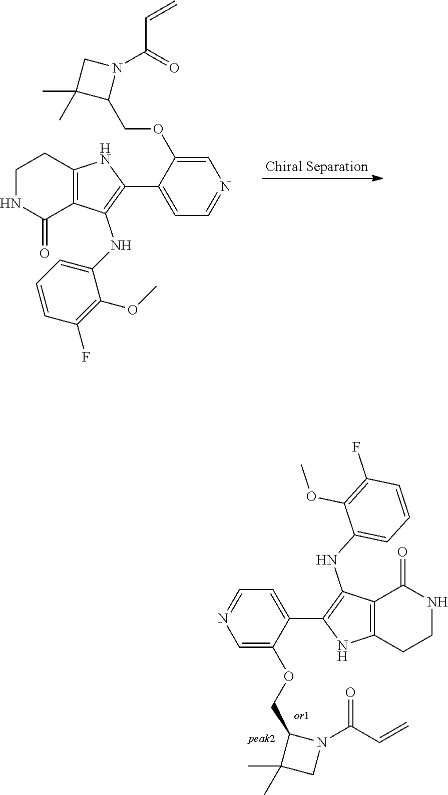 [SMILES: C=CC(=O)N1CC(C)(C)C1COc1cnccc1-c1[nH]c2c(c1Nc1cccc(F)c1OC)C(=O)NCC2 ; C=CC(=O)N1CC(C)(C)[C@H]1COc1cnccc1-c1[nH]c2c(c1Nc1cccc(F)c1OC)C(=O)NCC2]

The crude product 2-(3-{[3,3-dimethyl-1-(prop-2-enoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (40 mg, 0.077 mmol, 1 equiv) was purified by Prep-Chiral-HPLC with the following conditions (Column: CHIRALPAK IG, 2*25 cm, 5 m; Mobile Phase A: Hex:DCM=3:1(0.5% 2M NH₃-MeOH)—HPLC, Mobile Phase B: EtOH—HPLC; Flow rate: 20 mL/min; Gradient: 20% B to 20% B in 20 min; Wave Length: 220/254 nm; RT1(min): 13.70; RT2(min): 19.36; Sample Solvent: ETOH:DCM=1:1; Injection Volume: 2 mL; Number Of Runs: 2) to afford rel-2-(3-{[(2R)-3,3-dimethyl-1-(prop-2-enoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (12.7 mg, 31.59%) as a off-white solid.

LC-MS: (M+H)⁺ found 520.1.

¹H NMR (400 MHz, DMSO-d6) δ 8.38 (s, 1H), 7.95 (d, J=5.0 Hz, 1H), 7.39 (s, 1H), 7.32 (d, J=5.1 Hz, 1H), 6.79 (s, 1H), 6.57 (q, J=7.7 Hz, 1H), 6.43 (ddd, J=10.0, 8.4, 1.5 Hz, 1H), 6.34 (dd, J=17.0, 10.2 Hz, 1H), 6.22-6.13 (m, 1H), 6.00 (dt, J=8.2, 1.4 Hz, 1H), 4.59-4.50 (m, 1H), 4.45-4.35 (m, 2H), 3.91 (d, J=1.0 Hz, 5H), 3.40 (td, J=6.9, 2.6 Hz, 2H), 2.92 (q, J=4.7, 3.1 Hz, 2H), 1.27 (s, 3H), 1.22 (d, J=4.1 Hz, 3H).

### Example 209. rel-2-(3-{[(2R)-1-(but-2-ynoyl)-3,3-dimethylazetidin-2-yl]methoxy}pyridin-4-yl)-3-[(3-fluoro-2-methoxyphenyl)amino]-1H,5H,6H,7H-pyrrolo[3,2-c]pyridin-4-one (compound 355)

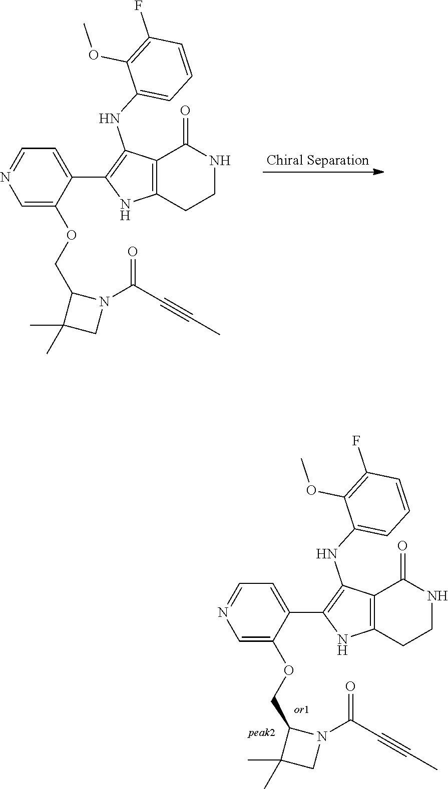 [SMILES: CC#CC(=O)N1CC(C)(C)C1COc1cnccc1-c1[nH]c2c(c1Nc1cccc(F)c1OC)C(=O)NCC2 ; CC#CC(=O)N1CC(C)(C)[C@H]1COc1cnccc1-c1[nH]c2c(c1Nc1cccc(F)c1OC)C(=O)NCC2]

The crude product 2-(3-{[1-(but-2-ynoyl)-3,3-dimethylazetidin-2-yl]methoxy}pyridin-4-yl)-3-[(3-fluoro-2-methoxyphenyl)amino]-1H,5H,6H,7H-pyrrolo[3,2-c]pyridin-4-one (20 mg, 0.038 mmol, 1 equiv) was purified by Prep-Chiral-HPLC with the following conditions (Column: CHIRALPAK IA-3, 4.6*50 mm 3 um; Mobile Phase A: Hex (0.1% DEA):EtOH=50:50; Flow rate: 1 mL/min; Gradient: 0% B to 0% B; Injection Volume: 5 ul mL) to afford rel-2-(3-{[(2R)-1-(but-2-ynoyl)-3,3-dimethylazetidin-2-yl]methoxy}pyridin-4-yl)-3-[(3-fluoro-2-methoxyphenyl)amino]-1H,5H,6H,7H-pyrrolo[3,2-c]pyridin-4-one (5 mg, 24.88%) as a light yellow solid.

LC-MS: (M+H)⁺ found 532.15.

¹H NMR (400 MHz, DMSO-d6) δ 8.38 (s, 1H), 7.95 (s, 1H), 7.36 (s, 1H), 7.31 (d, J=5.1 Hz, 1H), 6.80 (s, 1H), 6.56 (d, J=7.9 Hz, 1H), 6.43 (t, J=9.7 Hz, 1H), 5.99 (dt, J=8.3, 1.4 Hz, 1H), 4.56 (dd, J=10.2, 7.4 Hz, 1H), 4.40 (dd, J=10.2, 4.5 Hz, 1H), 4.33 (dd, J=7.4, 4.4 Hz, 1H), 3.91 (s, 5H), 3.39 (td, J=6.9, 2.6 Hz, 2H), 2.86 (s, 2H), 1.98 (s, 3H), 1.26 (s, 3H), 1.19 (s, 3H).

### Example 210. rel-2-(3-{[(2R)-1-(but-2-ynoyl)-3,3-dimethylazetidin-2-yl]methoxy}pyridin-4-yl)-3-[(3-fluoro-2-methoxyphenyl)amino]-1H,5H,6H,7H-pyrrolo[3,2-c]pyridin-4-one (compound 325)

### 210.1. Synthesis of 2-(3-{[1-(but-2-ynoyl)-3,3-dimethylazetidin-2-yl]methoxy}pyridin-4-yl)-3-[(3-fluoro-2-methoxyphenyl)amino]-1H,5H,6H,7H-pyrrolo[3,2-c]pyridin-4-one

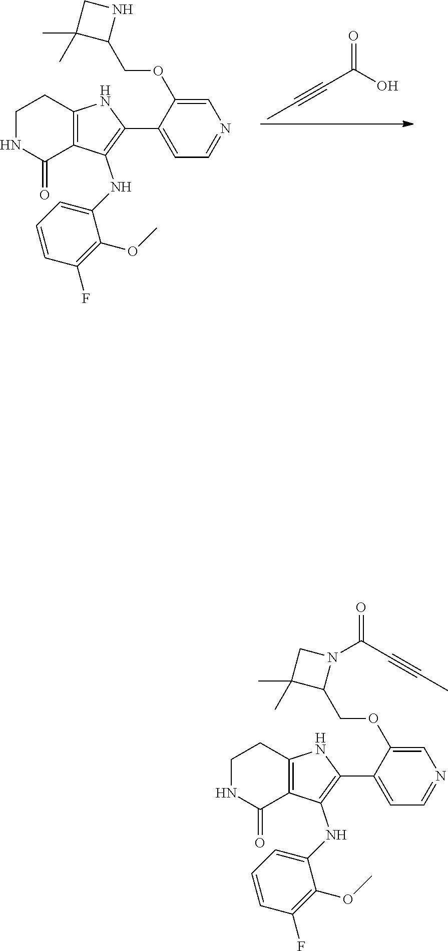

To a stirred mixture of 2-{3-[(3,3-dimethylazetidin-2-yl)methoxy]pyridin-4-yl}-3-[(3-fluoro-2-methoxyphenyl)amino]-1H,5H,6H,7H-pyrrolo[3,2-c]pyridin-4-one (100 mg, 0.215 mmol, 1 equiv) and T₃P (0.5 mL, 1.571 mmol, 7.32 equiv) in Pyridine (0.5 mL) and THF (1 mL) was added 2-butynoic acid (21.67 mg, 0.258 mmol, 1.2 equiv) dropwise at 0° C. The resulting mixture was stirred for 3 hours at RT. The mixture was neutralized to pH 7 with NaHCO₃. The resulting mixture was extracted with EA (3×15 mL). The combined organic layers were washed with brine (1×1 30 mL), dried over anhydrous Na2SO4. After filtration, the filtrate was concentrated under reduced pressure. The residue was purified by Prep-TLC (DCM:MeOH 20) to afford 2-(3-{[1-(but-2-ynoyl)-3,3-dimethylazetidin-2-yl]methoxy}pyridin-4-yl)-3-[(3-fluoro-2-methoxyphenyl)amino]-1H,5H,6H,7H-pyrrolo[3,2-c]pyridin-4-one (40 mg, 35.03%) as a light yellow solid.

LC-MS: (M+H)⁺ found 532.25.

### 210.2. Synthesis of rel-2-(3-{[(2R)-1-(but-2-ynoyl)-3,3-dimethylazetidin-2-yl]methoxy}pyridin-4-yl)-3-[(3-fluoro-2-methoxyphenyl)amino]-1H,5H,6H,7H-pyrrolo[3,2-c]pyridin-4-one

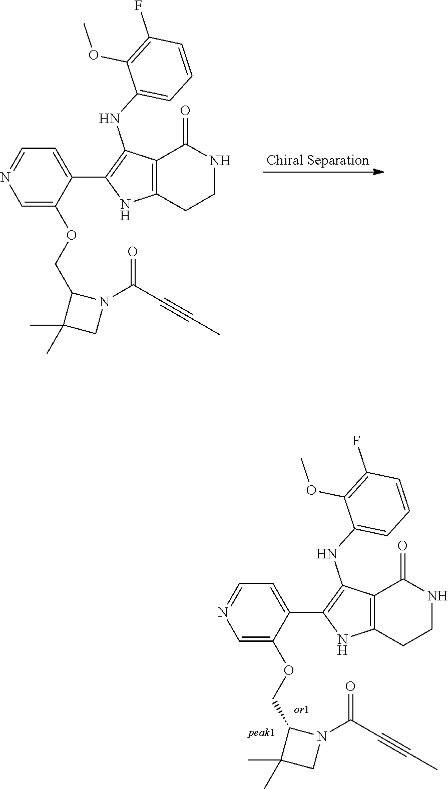 [SMILES: CC#CC(=O)N1CC(C)(C)C1COc1cnccc1-c1[nH]c2c(c1Nc1cccc(F)c1OC)C(=O)NCC2 ; CC#CC(=O)N1CC(C)(C)[C@@H]1COc1cnccc1-c1[nH]c2c(c1Nc1cccc(F)c1OC)C(=O)NCC2]

The crude product 2-(3-{[1-(but-2-ynoyl)-3,3-dimethylazetidin-2-yl]methoxy}pyridin-4-yl)-3-[(3-fluoro-2-methoxyphenyl)amino]-1H,5H,6H,7H-pyrrolo[3,2-c]pyridin-4-one (20 mg, 0.038 mmol, 1 equiv) was purified by Prep-Chiral-HPLC with the following conditions (Column: CHIRALPAK IA-3, 4.6*50 mm 3 um; Mobile Phase A: Hex (0.1% DEA):EtOH=50:50; Flow rate: 1 mL/min; Gradient: 0% B to 0% B; Injection Volume: 5 ul mL) to afford rel-2-(3-{[(2R)-1-(but-2-ynoyl)-3,3-dimethylazetidin-2-yl]methoxy}pyridin-4-yl)-3-[(3-fluoro-2-methoxyphenyl)amino]-1H,5H,6H,7H-pyrrolo[3,2-c]pyridin-4-one (5 mg, 24.78%) as a light yellow solid.

LC-MS: (M+H)⁺ found 532.15.

¹H NMR (400 MHz, DMSO-d6) δ 8.38 (s, 1H), 7.95 (s, 1H), 7.36 (s, 1H), 7.31 (d, J=5.1 Hz, 1H), 6.80 (s, 1H), 6.56 (d, J=7.9 Hz, 1H), 6.43 (t, J=9.7 Hz, 1H), 5.99 (dt, J=8.3, 1.4 Hz, 1H), 4.56 (dd, J=10.2, 7.4 Hz, 1H), 4.40 (dd, J=10.2, 4.5 Hz, 1H), 4.33 (dd, J=7.4, 4.4 Hz, 1H), 3.91 (s, 5H), 3.39 (td, J=6.9, 2.6 Hz, 2H), 2.86 (s, 2H), 1.98 (s, 3H), 1.26 (s, 3H), 1.19 (s, 3H).

### Example 211. 2-(3-{[(2R)-3,3-dimethyl-1-(prop-2-enoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (compound 324)

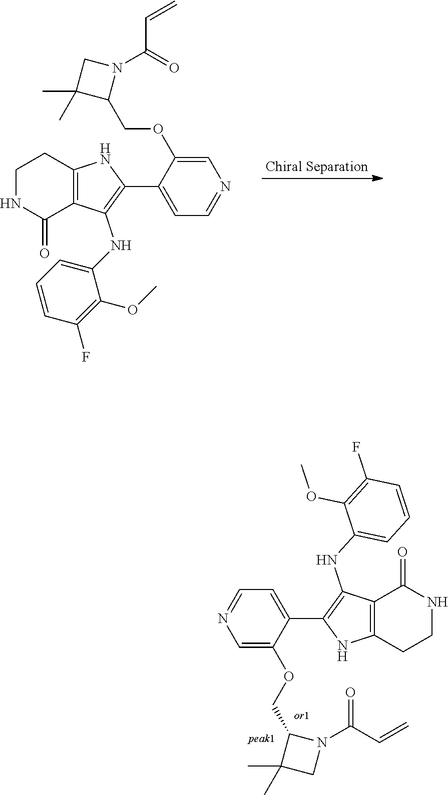 [SMILES: C=CC(=O)N1CC(C)(C)C1COc1cnccc1-c1[nH]c2c(c1Nc1cccc(F)c1OC)C(=O)NCC2 ; C=CC(=O)N1CC(C)(C)[C@@H]1COc1cnccc1-c1[nH]c2c(c1Nc1cccc(F)c1OC)C(=O)NCC2]

The crude product 2-(3-{[3,3-dimethyl-1-(prop-2-enoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (40 mg, 0.077 mmol, 1 equiv) was purified by Prep-Chiral-HPLC with the following conditions (Column: CHIRALPAK IG, 2*25 cm, 5 m; Mobile Phase A: Hex:DCM=3:1(0.5% 2M NH₃-MeOH)—HPLC, Mobile Phase B: EtOH—HPLC; Flow rate: 20 mL/min; Gradient: 20% B to 20% B in 20 min; Wave Length: 220/254 nm; RT1(min): 13.70; RT2(min): 19.36; Sample Solvent: ETOH:DCM=1:1; Injection Volume: 2 mL; Number Of Runs: 2) to afford 2-(3-{[(2R)-3,3-dimethyl-1-(prop-2-enoyl)azetidin-2-yl]methoxy}pyridin-4-yl)-3-[(3-fluoro-2-methoxyphenyl)amino]-1H,5H,6H,7H-pyrrolo[3,2-c]pyridin-4-one (11.5 mg, 28.61%) as a off-white solid.

LC-MS: (M+H)⁺ found 520.1.

¹H NMR (400 MHz, DMSO-d6) δ 8.38 (s, 1H), 7.95 (d, J=5.0 Hz, 1H), 7.39 (s, 1H), 7.32 (d, J=5.1 Hz, 1H), 6.79 (s, 1H), 6.57 (q, J=7.7 Hz, 1H), 6.43 (ddd, J=10.0, 8.4, 1.5 Hz, 1H), 6.34 (dd, J=17.0, 10.2 Hz, 1H), 6.22-6.13 (m, 1H), 6.00 (dt, J=8.2, 1.4 Hz, 1H), 4.59-4.50 (m, 1H), 4.45-4.35 (m, 2H), 3.91 (d, J=1.0 Hz, 5H), 3.40 (td, J=6.9, 2.6 Hz, 2H), 2.92 (q, J=4.7, 3.1 Hz, 2H), 1.27 (s, 3H), 1.22 (d, J=4.1 Hz, 3H).

### Example 212. 3-[(3-chloro-2-methoxyphenyl)amino]-2-(3-{[(2S)-2-methylazetidin-2-yl]methoxy}pyridin-4-yl)-1H,5H,6H,7H-pyrrolo[3,2-c]pyridin-4-one (compound 510)

### 212.1. Synthesis of tert-butyl 2-{[(4-bromopyridin-3-yl)oxy]methyl}-2-methylazetidine-1-carboxylate

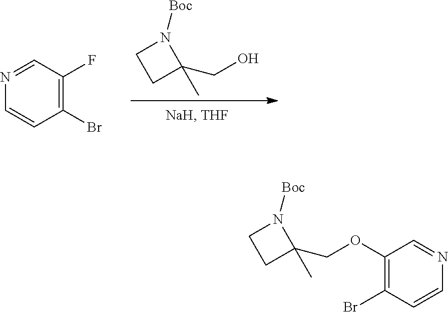

To a solution of tert-butyl 2-(hydroxymethyl)-2-methylazetidine-1-carboxylate (600 mg, 2.981 mmol, 1 equiv) in THF (3.0 mL) was added sodium hydride (60% in oil, 107.31 mg, 4.471 mmol, 1.5 equiv) at 0 degrees C. The mixture was stirred for 15 min. 4-bromo-3-fluoropyridine (629.57 mg, 3.577 mmol, 1.20 equiv) was added and the mixture was allowed to warm to RT and stirred for 2 h. The reaction mixture was quenched by water and extracted with DCM (3*25 mL). The residue was purified by silica gel column chromatography, eluted with DCM:MeOH (15:1) to afford tert-butyl 2-{[(4-bromopyridin-3-yl)oxy]methyl}-2-methylazetidine-1-carboxylate (500 mg, 51.23%) as a white solid.

### 212.2. Synthesis of tert-butyl2-methyl-2-{[(4-{4-oxo-1H,5H,6H,7H-pyrrolo[3,2-c]pyridin-2-yl}pyridin-3-yl)oxy]methyl}azetidine-1-carboxylate

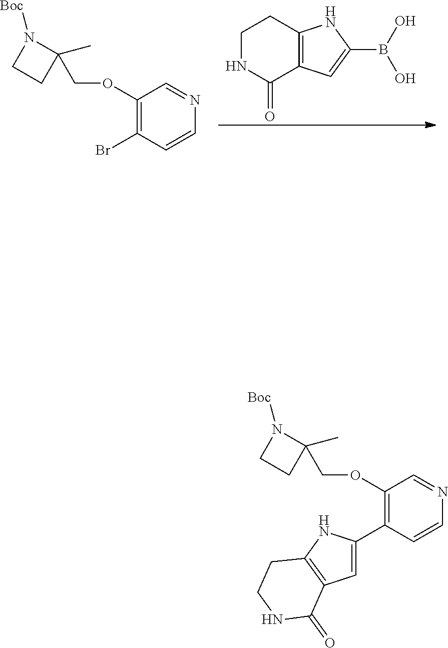 [SMILES: CC(C)(C)OC(=O)N1CCC1(C)COc1cnccc1Br ; O=C1NCCc2[nH]c(B(O)O)cc21 ; CC(C)(C)OC(=O)N1CCC1(C)COc1cnccc1-c1cc2c([nH]1)CCNC2=O]

To stirred a solution of tert-butyl 2-{[(4-bromopyridin-3-yl)oxy]methyl}-2-methylazetidine-1-carboxylate (50 mg, 0.140 mmol, 1 equiv) and 2-(4,4,5,5-tetramethyl-1,3,2-dioxaborolan-2-yl)-1H,5H,6H,7H-pyrrolo[3,2-c]pyridin-4-one (55.03 mg, 0.210 mmol, 1.5 equiv) in dioxane (1.5 mL) were added K₂CO₃ (48.36 mg, 0.350 mmol, 2.5 equiv) and Pd(dppf)C12 (15.36 mg, 0.021 mmol, 0.15 equiv). After stirring for 3h at 80° C. under a nitrogen atmosphere, the resulting mixture was concentrated under reduced pressure. The residue was purified by Prep-TLC/silica gel column chromatography, eluted with DCM:MeOH (25:1) to afford tert-butyl2-methyl-2-{[(4-{4-oxo-1H,5H,6H,7H-pyrrolo[3,2-c]pyridin-2-yl}pyridin-3-yl)oxy]methyl}azetidine-1-carboxylate (29 mg, 41.23%) as a yellow solid.

LC-MS: (M+H)⁺ found 426.05.

### 212.3. Synthesis of tert-butyl 2-{[(4-{3-iodo-4-oxo-1H,5H,6H,7H-pyrrolo[3,2-c]pyridin-2-yl} pyridine-3-yl)oxy]methyl}-2-methylazetidine-1-carboxylate

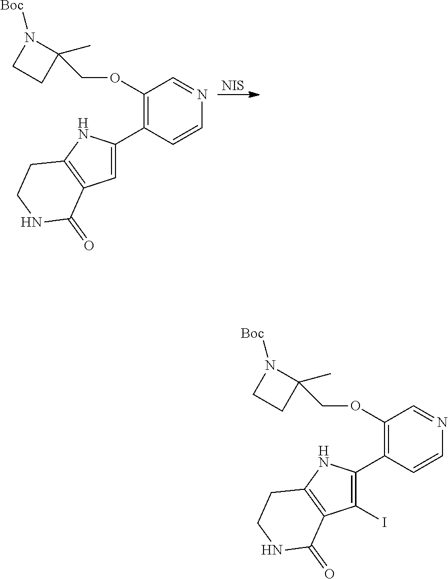

To a stirred solution of tert-butyl 2-methyl-2-{[(4-{4-oxo-1H,5H,6H,7H-pyrrolo[3,2-c]pyridin-2-yl}pyridin-3-yl)oxy]methyl}azetidine-1-carboxylate (1000 mg, 2.424 mmol, 1 equiv) in DMF (15 mL) was added NIS (654.52 mg, 2.909 mmol, 1.2 equiv) in portions at RT under N₂ atmosphere. The reaction was quenched by the addition of Na₂SO₃ (50 ml 1 mol/L) at 0° C. The precipitated solids were collected by filtration and washed with THF (2×2 10 ml). The residue was purified by trituration with EA (15 ml) to afford tert-butyl 2-{[(4-{3-iodo-4-oxo-1H,5H,6H,7H-pyrrolo[3,2-c]pyridin-2-yl}pyridine-3-yl)oxy]methyl}-2-methylazetidine-1-carboxylate (1300 mg, 99.60%) as a light yellow solid.

LC-MS: (M−H)⁻ found 450.05.

### 212.4. Synthesis of tert-butyl 2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-2-methylazetidine-1-carboxylate

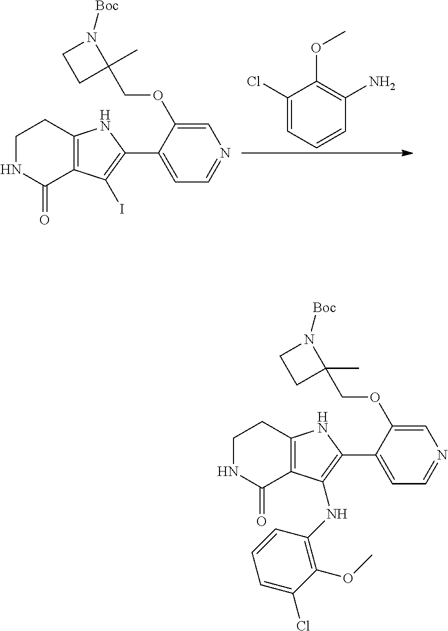

To a solution of tert-butyl 2-{[(4-{3-iodo-4-oxo-1H,5H,6H,7H-pyrrolo[3,2-c]pyridin-2-yl}pyridin-3-yl)oxy]methyl}-2-methylazetidine-1-carboxylate (150 mg, 0.279 mmol, 1 equiv) and 3-chloro-2-methoxyaniline (87.82 mg, 0.558 mmol, 2 equiv) in DMF (3.5 mL) were added Cs₂CO₃ (226.94 mg, 0.698 mmol, 2.5 equiv) and EPhos Pd G4 (51.18 mg, 0.056 mmol, 0.2 equiv) and EPhos (29.80 mg, 0.056 mmol, 0.2 equiv). After stirring for 2 h at 50° C. under a nitrogen atmosphere, the resulting mixture was concentrated under reduced pressure. The residue was purified by Prep-TLC/silica gel column chromatography, eluted with DCM:MeOH (20:1) to afford tert-butyl 2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-2-methylazetidine-1-carboxylate (75 mg, 37.91%) as a light yellow solid.

LC-MS: (M+H)⁺ found 541.10.

### 212.5. Synthesis of 3-[(3-chloro-2-methoxyphenyl)amino]-2-{3-[(2-methylazetidin-2-yl)methoxy]pyridin-4-yl}-1H,5H,6H,7H-pyrrolo[3,2-c]pyridin-4-one

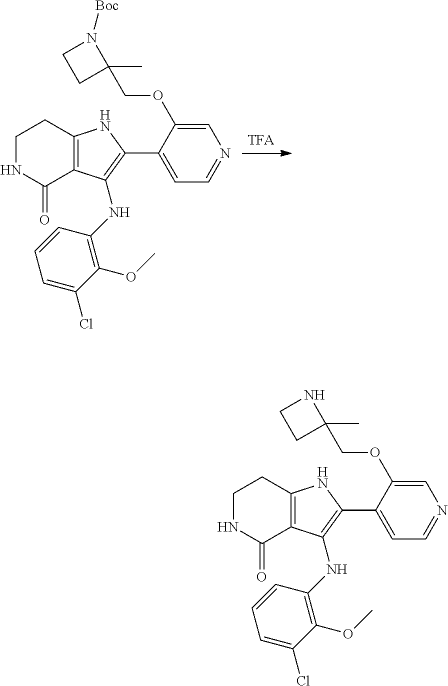 [SMILES: COc1c(Cl)cccc1Nc1c(-c2ccncc2OCC2(C)CCN2C(=O)OC(C)(C)C)[nH]c2c1C(=O)NCC2 ; COc1c(Cl)cccc1Nc1c(-c2ccncc2OCC2(C)CCN2)[nH]c2c1C(=O)NCC2]

To a stirred solution of tert-butyl 2-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-2-methylazetidine-1-carboxylate (75 mg, 0.132 mmol, 1 equiv) in DCM (2.5 mL) was added TFA (1.5 mL) dropwise at 0° C. under N₂ atmosphere. The mixture was basified to pH 8.5 with NaHCO₃. The resulting mixture was extracted with EA (2×30 ml). The combined organic layers were washed with brine (2×2 10 ml), dried over anhydrous Na₂SO₄. After filtration, the filtrate was concentrated under reduced pressure to afford 3-[(3-chloro-2-methoxyphenyl)amino]-2-{3-[(2-methylazetidin-2-yl)methoxy]pyridin-4-yl}-1H,5H,6H,7H-pyrrolo[3,2-c]pyridin-4-one (500 mg, 51.23%) as a light yellow solid.

LC-MS: (M+H)⁺ found 441.10.

### 212.5. Synthesis of 3-[(3-chloro-2-methoxyphenyl)amino]-2-(3-{[2-methyl-1-(prop-2-enoyl)azetidin-2-yl]methoxy}pyridin-4-yl)-1H,5H,6H,7H-pyrrolo[3,2-c]pyridin-4-one

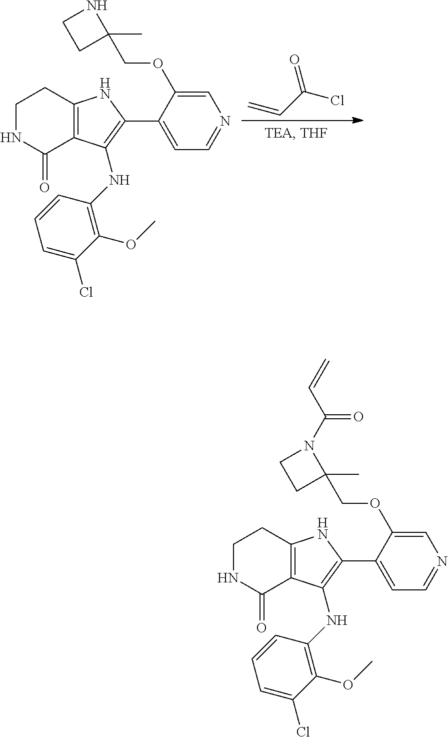

A solution of 3-[(3-chloro-2-methoxyphenyl) amino]-2-{3-[(2-methylazetidin-2-yl)methoxy]pyridin-4-yl}-1H,5H,6H,7H-pyrrolo[3,2-c]pyridin-4-one (45 mg, 0.096 mmol, 1 equiv) and NaHCO₃(20.20 mg, 0.240 mmol, 2.5 equiv) in THF (2 mL) and H₂O (2 mL) was added acryloyl chloride (7.83 mg, 0.086 mmol, 0.9 equiv) dropwise at 0° C. under N₂ atmosphere. The resulting mixture was extracted with EA (2×20 ml). The combined organic layers were washed with brine (2×2 20 ml), dried over anhydrous Na₂SO₄. After filtration, the filtrate was concentrated under reduced pressure and purified by prep-TLC (DCM/MeOH=15:1) to afford 3-[(3-chloro-2-methoxyphenyl)amino]-2-(3-{[2-methyl-1-(prop-2-enoyl)azetidin-2-yl]methoxy}pyridin-4-yl)-1H,5H,6H,7H-pyrrolo[3,2-c]pyridin-4-one (30 mg, 60%) as a light yellow solid.

LC-MS: (M+H)⁺ found 501.10.

### 212.5. Synthesis of 3-[(3-chloro-2-methoxyphenyl)amino]-2-(3-{[(2S)-2-methylazetidin-2-yl]methoxy}pyridin-4-yl)-1H,5H,6H,7H-pyrrolo[3,2-c]pyridin-4-one

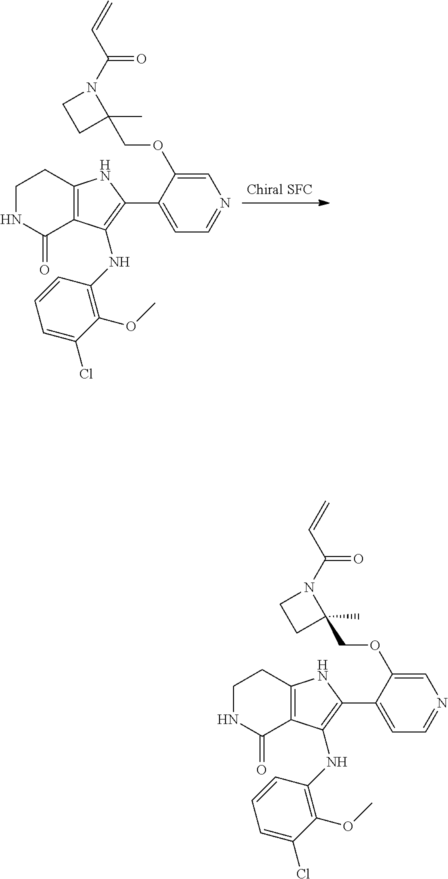 [SMILES: C=CC(=O)N1CCC1(C)COc1cnccc1-c1[nH]c2c(c1Nc1cccc(Cl)c1OC)C(=O)NCC2 ; C=CC(=O)N1CC[C@]1(C)COc1cnccc1-c1[nH]c2c(c1Nc1cccc(Cl)c1OC)C(=O)NCC2]

The crude product (50 mg) was purified by Prep-HPLC with the following conditions (Column: CHIRALPAK IG-3, 4.6*50 mm, 3 um; Mobile Phase A: (Hex:DCM=3:1)(0.1% DEA):EtOH=80:20; Flow rate: 1 mL/min; Gradient: 0% B to 0% B; Injection Volume: 5 ul mL) to afford 3-[(3-chloro-2-methoxyphenyl)amino]-2-(3-{[(2S)-2-methylazetidin-2-yl]methoxy}pyridin-4-yl)-1H,5H,6H,7H-pyrrolo[3,2-c]pyridin-4-one (4.6 mg, 10.22%) as a yellow solid.

LC-MS: (M+H)⁺ found 501.10.

¹H NMR (400 MHz, DMSO-d6) δ 11.93 (s, 1H), 8.39 (s, 1H), 8.00 (d, J=5.0 Hz, 1H), 7.47 (s, 1H), 7.34 (d, J=5.1 Hz, 1H), 7.12 (s, 1H), 6.76-6.63 (m, 2H), 6.34-6.07 (m, 3H), 5.69 (dd, J=9.9, 2.5 Hz, 1H), 4.55 (d, J=10.2 Hz, 1H), 4.31 (d, J=10.2 Hz, 1H), 4.23-4.03 (m, 2H), 3.89 (s, 3H), 3.42 (s, 3H), 3.05-2.76 (m, 2H), 2.41-2.29 (m, 1H), 2.12 (q, J=10.0 Hz, 1H), 1.67 (s, 3H), 1.57 (s, 1H).

### Example 213. 3-[(3-chloro-2-methoxyphenyl)amino]-2-(3-{[(2R)-2-methylazetidin-2-yl]methoxy}pyridin-4-yl)-1H,5H,6H,7H-pyrrolo[3,2-c]pyridin-4-one (compound 511)

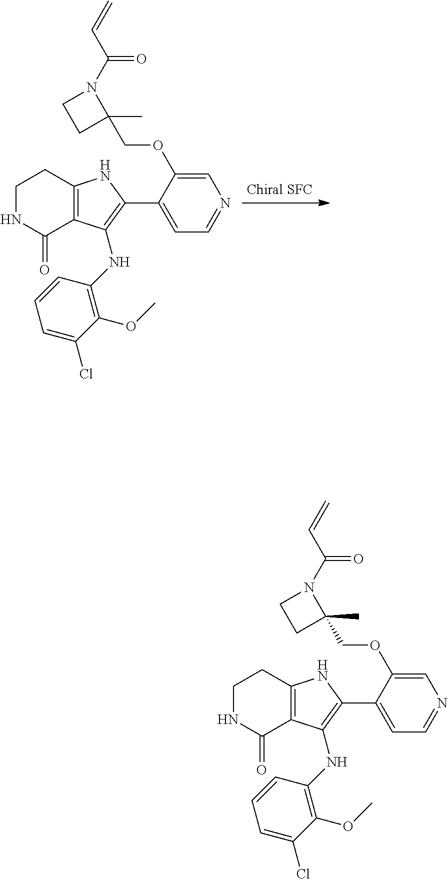 [SMILES: C=CC(=O)N1CCC1(C)COc1cnccc1-c1[nH]c2c(c1Nc1cccc(Cl)c1OC)C(=O)NCC2 ; C=CC(=O)N1CC[C@@]1(C)COc1cnccc1-c1[nH]c2c(c1Nc1cccc(Cl)c1OC)C(=O)NCC2]

The crude product (50 mg) was purified by Prep-HPLC with the following conditions (Column: CHIRALPAK IG-3, 4.6*50 mm, 3 um; Mobile Phase A: (Hex:DCM=3:1)(0.1% DEA):EtOH=80:20; Flow rate: 1 mL/min; Gradient: 0% B to 0% B; Injection Volume: 5 ul mL) to afford 3-[(3-chloro-2-methoxyphenyl)amino]-2-(3-{[(2R)-2-methylazetidin-2-yl]methoxy}pyridin-4-yl)-1H,5H,6H,7H-pyrrolo[3,2-c]pyridin-4-one (5.6 mg, 11.86%) as a yellow solid.

LC-MS: (M+H)⁺ found 522.50.

¹H NMR (400 MHz, DMSO-d6) δ 11.93 (s, 1H), 8.39 (s, 1H), 8.00 (d, J=5.0 Hz, 1H), 7.47 (s, 1H), 7.34 (d, J=5.1 Hz, 1H), 7.12 (s, 1H), 6.76-6.63 (m, 2H), 6.34-6.07 (m, 3H), 5.69 (dd, J=9.9, 2.5 Hz, 1H), 4.55 (d, J=10.2 Hz, 1H), 4.31 (d, J=10.2 Hz, 1H), 4.23-4.03 (m, 2H), 3.89 (s, 3H), 3.42 (s, 3H), 3.05-2.76 (m, 2H), 2.41-2.29 (m, 1H), 2.12 (q, J=10.0 Hz, 1H), 1.67 (s, 3H), 1.57 (s, 1H).

### Example 214. rel-3-[(2-ethyl-3-fluorophenyl)amino]-2-(3-{[(2R)-2-methyl-1-(prop-2-enoyl)azetidin-2-yl]methoxy}pyridin-4-yl)-1H,5H,6H,7H-pyrrolo[3,2-c]pyridin-4-one (compound 359)

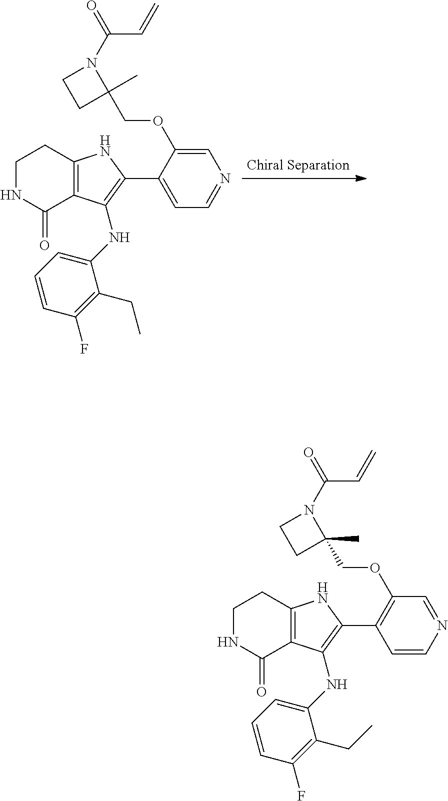 [SMILES: C=CC(=O)N1CCC1(C)COc1cnccc1-c1[nH]c2c(c1Nc1cccc(F)c1CC)C(=O)NCC2 ; C=CC(=O)N1CC[C@@]1(C)COc1cnccc1-c1[nH]c2c(c1Nc1cccc(F)c1CC)C(=O)NCC2]

The 3-[(2-ethyl-3-fluorophenyl)amino]-2-(3-{[2-methyl-1-(prop-2-enoyl)azetidin-2-yl]methoxy}pyridin-4-yl)-1H,5H,6H,7H-pyrrolo[3,2-c]pyridin-4-one (15 mg, 0.030 mmol, 1 equiv) was purified by the following Column: CHIRALPAK IG-3, 4.6*50 mm; 3 um; Mobile Phase A: (Hex:DCM=3:1)(0.1% DEA):EtOH=70:30; Flow rate: 1 mL/min; Gradient: 0% B to 0% B; Injection Volume: 5 ul mL to afford rel-3-[(2-ethyl-3-fluorophenyl)amino]-2-(3-{[(2R)-2-methyl-1-(prop-2-enoyl)azetidin-2-yl]methoxy}pyridin-4-yl)-1H,5H,6H,7H-pyrrolo[3,2-c]pyridin-4-one (1.2 mg, 7.90%) as a yellow solid.

LC-MS: M found 504.35.

¹H NMR (300 MHz, DMSO-d6) δ 12.01 (s, 1H), 8.38 (s, 1H), 7.99 (d, J=5.3 Hz, 1H), 7.50 (s, 1H), 7.29 (d, J=5.3 Hz, 1H), 7.19 (t, J=2.6 Hz, 1H), 6.78 (td, J=8.2, 6.7 Hz, 1H), 6.49 (t, J=8.9 Hz, 1H), 6.30 (dd, J=16.9, 9.8 Hz, 1H), 6.23-6.01 (m, 2H), 5.71 (dd, J=9.7, 2.7 Hz, 1H), 4.58 (d, J=10.2 Hz, 1H), 4.31 (d, J=10.3 Hz, 1H), 4.16 (dtd, J=24.4, 8.9, 6.1 Hz, 2H), 3.44 (d, J=2.5 Hz, 2H), 3.04-2.87 (m, 2H), 2.72 (q, J=7.4, 6.8 Hz, 2H), 2.38-2.25 (m, 1H), 2.21-2.04 (m, 1H), 1.69 (s, 3H), 1.29-1.12 (m, 3H).

### Example 215. rel-3-[(2-ethyl-3-fluorophenyl)amino]-2-(3-{[(2R)-2-methyl-1-(prop-2-enoyl)azetidin-2-yl]methoxy}pyridin-4-yl)-1H,5H,6H,7H-pyrrolo[3,2-c]pyridin-4-one (compound 309)

### 215.1. Synthesis of 2-ethenyl-1-fluoro-3-nitrobenzene

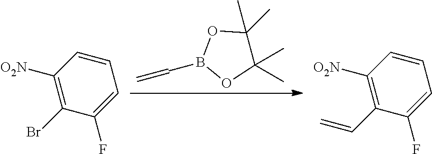

To a stirred solution of 2-bromo-1-fluoro-3-nitrobenzene (2 g, 9.091 mmol, 1 equiv) and 2-ethenyl-4,4,5,5-tetramethyl-1,3,2-dioxaborolane (2.10 g, 13.636 mmol, 1.5 equiv) in dioxane (20 mL) were added Pd(dppf)Cl₂ (0.67 g, 0.909 mmol, 0.1 equiv) and K₂CO₃ (2.51 g, 18.182 mmol, 2 equiv) at rt under N₂ atmosphere. Then, the solution was stirred at 80° C. for 3h. The resulting mixture was concentrated under reduced pressure and purified by silica gel column chromatography, eluted with PE/EA (100/1) to afford 2-ethenyl-1-fluoro-3-nitrobenzene (400 mg, 26.33%) as a colorless oil.

GC-MS: M+1 found: 167.9.

### 215.2. Synthesis of 2-ethyl-3-fluoroaniline

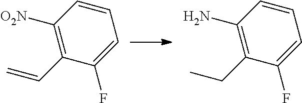

To a stirred solution of 2-ethenyl-1-fluoro-3-nitrobenzene (400 mg, 2.393 mmol, 1 equiv) in MeOH (20 mL) was added Pd/C (80 mg) at rt. Then, the solution was backfilled with H2 and the solution was stirred at rt for 1 h. The resulting mixture was filtered and the filtrate was concentrated under reduced pressure to afford 2-ethyl-3-fluoroaniline (90 mg, 27.02%) as a colorless oil.

GC-MS: M found: 139.0.

### 215.3. Synthesis of tert-butyl 2-{[(4-{3-[(2-ethyl-3-fluorophenyl)amino]-4-oxo-1H,5H,6H,7H-pyrrolo[3,2-c]pyridin-2-yl}pyridin-3-yl)oxy]methyl}-2-methylazetidine-1-carboxylate)

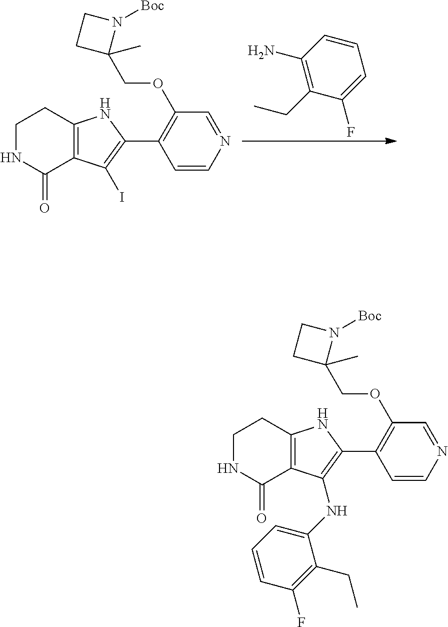

To a stirred solution of tert-butyl 2-{[(4-{3-iodo-4-oxo-1H,5H,6H,7H-pyrrolo[3,2-c]pyridin-2-yl}pyridin-3-yl)oxy]methyl}-2-methylazetidine-1-carboxylate (250 mg, 0.464 mmol, 1 equiv) and 2-ethyl-3-fluoroaniline (129.25 mg, 0.928 mmol, 2 equiv) in DMF (3.5 mL) were added EPhos Pd G4 (63.98 mg, 0.070 mmol, 0.15 equiv) and EPhos (37.25 mg, 0.070 mmol, 0.15 equiv) at rt under N₂ atmosphere. Then, the solution was stirred at 50° C. for 3h. The aqueous layer was extracted with EA (3×20 mL) and the extracts was concentrated under reduced pressure to afford tert-butyl 2-{[(4-{3-[(2-ethyl-3-fluorophenyl)amino]-4-oxo-1H,5H,6H,7H-pyrrolo[3,2-c]pyridin-2-yl}pyridin-3-yl)oxy]methyl}-2-methylazetidine-1-carboxylate) (113 mg, 44.3%) as a yellow oil.

LC-MS: M+H found: 550.0.

### 215.4. Synthesis of 3-[(2-ethyl-3-fluorophenyl)amino]-2-{3-[(2-methylazetidin-2-yl)methoxy]pyridin-4-yl}-1H,5H,6H,7H-pyrrolo[3,2-c]pyridin-4-one

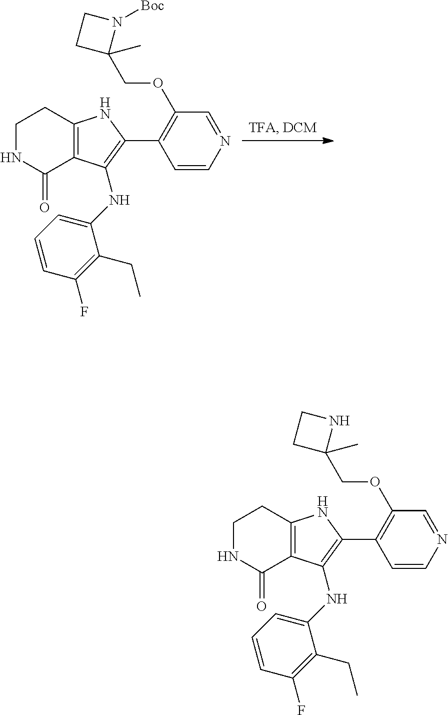 [SMILES: CCc1c(F)cccc1Nc1c(-c2ccncc2OCC2(C)CCN2C(=O)OC(C)(C)C)[nH]c2c1C(=O)NCC2 ; CCc1c(F)cccc1Nc1c(-c2ccncc2OCC2(C)CCN2)[nH]c2c1C(=O)NCC2]

To a stirred solution of tert-butyl 2-{[(4-{3-[(2-ethyl-3-fluorophenyl)amino]-4-oxo-1H,5H,6H,7H-pyrrolo[3,2-c]pyridin-2-yl}pyridin-3-yl)oxy]methyl}-2-methylazetidine-1-carboxylate (113 mg, 0.206 mmol, 1 equiv) in DCM (3 mL) was added TFA (1.5 mL) at 0° C. under N₂ atmosphere. Then, the solution was stirred at 0° C. for 2h. The resulting mixture was concentrated under reduced pressure to afford crude 3-[(2-ethyl-3-fluorophenyl)amino]-2-{3-[(2-methylazetidin-2-yl)methoxy]pyridin-4-yl}-1H,5H,6H,7H-pyrrolo[3,2-c]pyridin-4-one (92 mg, 99.55%) as a yellow oil.

LC-MS: M+H found: 450.2.

### 215.5. Synthesis of 3-[(2-ethyl-3-fluorophenyl)amino]-2-(3-{[2-methyl-1-(prop-2-enoyl)azetidin-2-yl]methoxy}pyridin-4-yl)-1H,5H,6H,7H-pyrrolo[3,2-c]pyridin-4-one

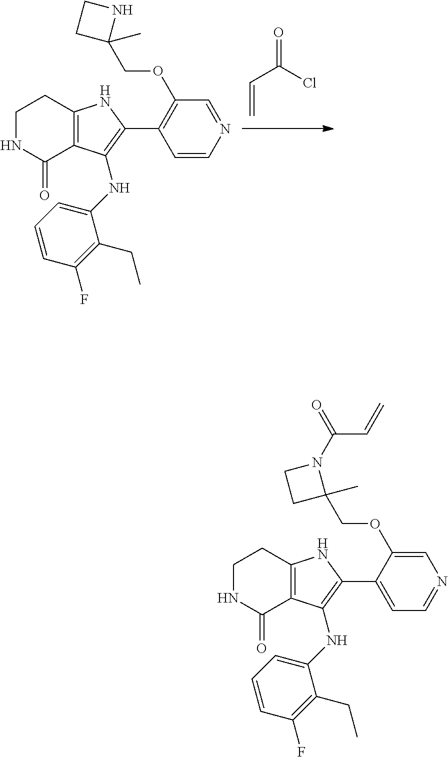

To a stirred solution of 3-[(2-ethyl-3-fluorophenyl)amino]-2-{3-[(2-methylazetidin-2-yl)methoxy]pyridin-4-yl}-1H,5H,6H,7H-pyrrolo[3,2-c]pyridin-4-one (113 mg, 0.251 mmol, 1 equiv) in THF (3 mL) were added NaHCO₃(2 mL) and acryloyl chloride (22.75 mg, 0.251 mmol, 1 equiv) at 0° C. under N₂ atmosphere. Then, the solution was stirred at 0° C. for 30 min. The aqueous layer was extracted with EA (3×10 mL) and the extracts was concentrated at reduced pressure. The residue was purified by Prep-TLC (DCM:MeOH=15:1) to afford 3-[(2-ethyl-3-fluorophenyl)amino]-2-(3-{[2-methyl-1-(prop-2-enoyl)azetidin-2-yl]methoxy}pyridin-4-yl)-1H,5H,6H,7H-pyrrolo[3,2-c]pyridin-4-one (41 mg, 32.39%) as a yellow solid.

LC-MS: M+H found: 504.25.

### 215.6. Synthesis of rel-3-[(2-ethyl-3-fluorophenyl)amino]-2-(3-{[(2R)-2-methyl-1-(prop-2-enoyl)azetidin-2-yl]methoxy}pyridin-4-yl)-1H,5H,6H,7H-pyrrolo[3,2-c]pyridin-4-one

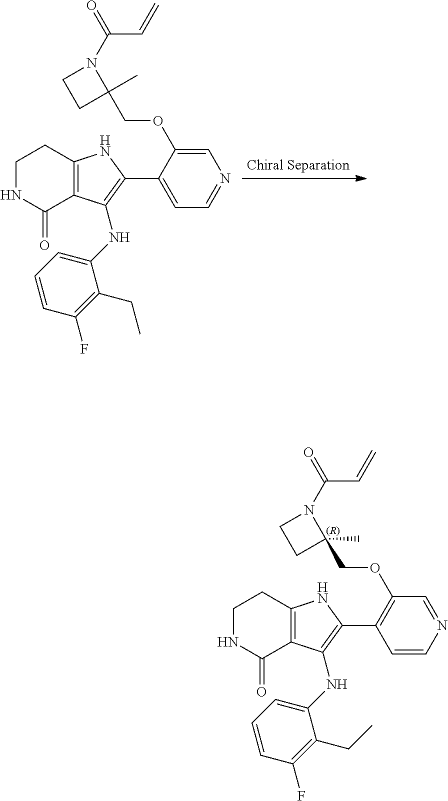

The 3-[(2-ethyl-3-fluorophenyl)amino]-2-(3-{[2-methyl-1-(prop-2-enoyl)azetidin-2-yl]methoxy}pyridin-4-yl)-1H,5H,6H,7H-pyrrolo[3,2-c]pyridin-4-one (15 mg, 0.030 mmol, 1 equiv) was purified by the following Column: CHIRALPAK IG-3, 4.6*50 mm; 3 um; Mobile Phase A: (Hex:DCM=3:1)(0.1% DEA):EtOH=70:30; Flow rate: 1 mL/min; Gradient: 0% B to 0% B; Injection Volume: 5 ul mL to afford rel-3-[(2-ethyl-3-fluorophenyl)amino]-2-(3-{[(2R)-2-methyl-1-(prop-2-enoyl)azetidin-2-yl]methoxy}pyridin-4-yl)-1H,5H,6H,7H-pyrrolo[3,2-c]pyridin-4-one (2.7 mg, 17.84%) as a yellow solid.

LC-MS: M+H found 504.35.

¹H NMR (300 MHz, DMSO-d6) δ 12.03 (s, 1H), 8.39 (s, 1H), 7.99 (d, J=5.4 Hz, 1H), 7.52 (s, 1H), 7.30 (d, J=5.4 Hz, 1H), 7.20 (q, J=2.4 Hz, 1H), 6.78 (td, J=8.2, 6.7 Hz, 1H), 6.50 (t, J=8.9 Hz, 1H), 6.44-6.01 (m, 3H), 5.71 (dd, J=9.7, 2.7 Hz, 1H), 4.58 (d, J=10.2 Hz, 1H), 4.31 (d, J=10.2 Hz, 1H), 4.16 (dtd, J=24.4, 8.9, 6.0 Hz, 2H), 3.44 (d, J=2.6 Hz, 2H), 2.97 (dt, J=8.7, 6.8 Hz, 2H), 2.72 (q, J=7.2 Hz, 2H), 2.38-2.26 (m, 1H), 2.14 (ddd, J=11.3, 9.4, 6.2 Hz, 1H), 1.69 (s, 3H), 1.30-1.12 (m, 3H).

### Example 216. 3-[(3-fluoro-2-methoxyphenyl)amino]-2-(3-{[(2R*,4R**)-4-methyl-1-(prop-2-enoyl)azetidin-2-yl]methoxy}pyridin-4-yl)-1H,5H,6H,7H-pyrrolo[3,2-c]pyridin-4-one (compound 333)

### 216.1. Synthesis of tert-butyl 2-(hydroxymethyl)-4-methylazetidine-1-carboxylate

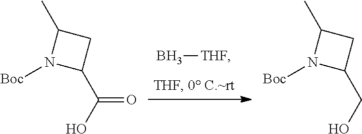

To a stirred solution of 1-(tert-butoxycarbonyl)-4-methylazetidine-2-carboxylic acid (1.9 g, 8.827 mmol, 1 equiv) in THF (19 mL) were added BH₃-THF (17.8 mL, 185.993 mmol, 21.07 equiv) dropwise at 0° C. under nitrogen atmosphere. The resulting mixture was stirred for overnight at room temperature under nitrogen atmosphere. The residue was purified by reverse flash chromatography with the following conditions: column, silica gel; mobile phase, MeCN in water, 10% to 50% gradient in 10 min; detector, UV 254 nm. to afford tert-butyl 2-(hydroxymethyl)-4-methylazetidine-1-carboxylate (1.27 g, 71.49%) as off-white liquid.

LCMS: [M+H]+ found 202.

### 216.2. Synthesis of afford tert-butyl 2-{[(4-bromopyridin-3-yl)oxy]methyl}-4-methylazetidine-1-carboxylate

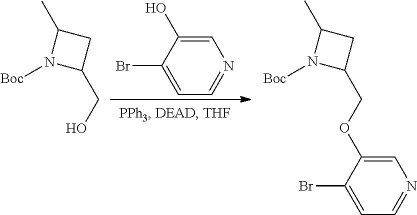

To a stirred mixture of tert-butyl 2-(hydroxymethyl)-4-methylazetidine-1-carboxylate (1.27 g, 6.310 mmol, 1 equiv) and 4-bromopyridin-3-ol (1.10 g, 6.310 mmol, 1 equiv) in THF (13 mL) was added PPh₃ (3.31 g, 12.620 mmol, 2 equiv) and DEAD (2.20 g, 12.620 mmol, 2 equiv) dropwise at 0° C. under nitrogen atmosphere. The resulting mixture was stirred for 1 h at room temperature under nitrogen atmosphere. The residue was purified by reverse flash chromatography with the following conditions: column, silica gel; mobile phase, MeCN in water, 10% to 50% gradient in 10 min; detector, UV 254 nm. to afford tert-butyl 2-{[(4-bromopyridin-3-yl)oxy]methyl}-4-methylazetidine-1-carboxylate (915 mg, 40.59%) as a brown liquid.

LCMS: [M+H]⁺ found 357.

### 216.3. Synthesis of tert-butyl 2-methyl-4-{[(4-{4-oxo-1H,5H,6H,7H-pyrrolo[3,2-c]pyridin-2-yl}pyridin-3-yl)oxy]methyl}azetidine-1-carboxylate

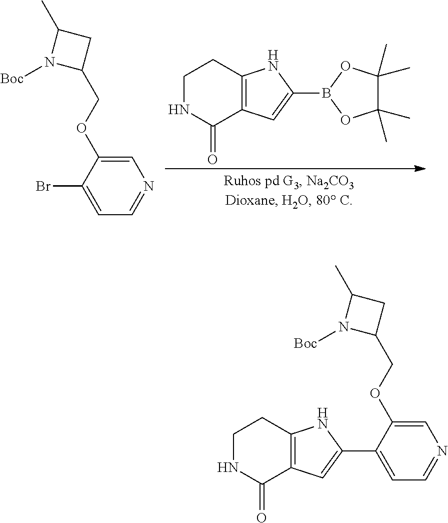 [SMILES: CC1CC(COc2cnccc2Br)N1C(=O)OC(C)(C)C ; CC1(C)OB(c2cc3c([nH]2)CCNC3=O)OC1(C)C ; CC1CC(COc2cnccc2-c2cc3c([nH]2)CCNC3=O)N1C(=O)OC(C)(C)C]

To a stirred mixture of tert-butyl 2-{[(4-bromopyridin-3-yl)oxy]methyl}-4-methylazetidine-1-carboxylate (915 mg, 2.561 mmol, 1 equiv) and 2-(4,4,5,5-tetramethyl-1,3,2-dioxaborolan-2-yl)-1H,5H,6H,7H-pyrrolo[3,2-c]pyridin-4-one (1.01 g, 3.841 mmol, 1.5 equiv) in Dioxane (10 mL) and H₂O (2 mL) was added Na₂CO₃ (814.39 mg, 7.683 mmol, 3 equiv) and RuPhos Palladacycle Gen. 3 (428.43 mg, 0.512 mmol, 0.2 equiv) in portions at room temperature under nitrogen atmosphere. The resulting mixture was stirred for overnight at 80° C. under nitrogen atmosphere. The residue was purified by reverse flash chromatography with the following conditions: column, silica gel; mobile phase, MeCN in water, 10% to 50% gradient in 10 min; detector, UV 254 nm. to afford tert-butyl 2-methyl-4-{[(4-{4-oxo-1H,5H,6H,7H-pyrrolo[3,2-c]pyridin-2-yl}pyridin-3-yl)oxy]methyl}azetidine-1-carboxylate (800 mg, 75.72%) as a yellow oil.

LCMS: [M+H]⁺ found 414.

### 216.4. Synthesis of tert-butyl 2-{[(4-{3-iodo-4-oxo-1H,5H,6H,7H-pyrrolo[3,2-c]pyridin-2-yl}pyridin-3-yl)oxy]methyl}-4-methylazetidine-1-carboxylate

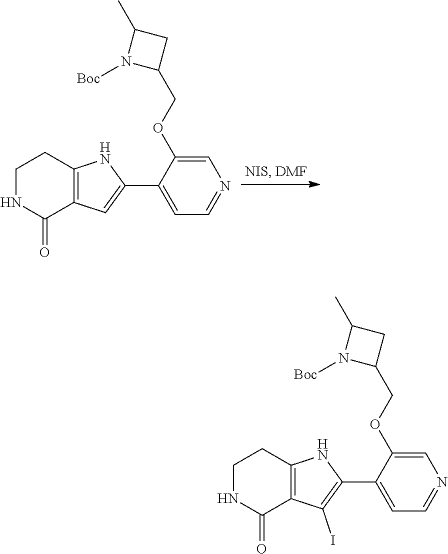 [SMILES: CC1CC(COc2cnccc2-c2cc3c([nH]2)CCNC3=O)N1C(=O)OC(C)(C)C ; CC1CC(COc2cnccc2-c2[nH]c3c(c2I)C(=O)NCC3)N1C(=O)OC(C)(C)C]

To a stirred solution of tert-butyl 2-methyl-4-{[(4-{4-oxo-1H,5H,6H,7H-pyrrolo[3,2-c]pyridin-2-yl}pyridin-3-yl)oxy]methyl}azetidine-1-carboxylate (800 mg, 1.939 mmol, 1 equiv) in DMF (8 mL) was added NIS (523.61 mg, 2.327 mmol, 1.2 equiv) in portions at 0° C. under nitrogen atmosphere. The resulting mixture was stirred for 4 h at room temperature under nitrogen atmosphere. The reaction was quenched with Water at room temperature. The precipitated solids were collected by filtration and washed with water (3×50 mL). to afford tert-butyl 2-{[(4-{3-iodo-4-oxo-1H,5H,6H,7H-pyrrolo[3,2-c]pyridin-2-yl}pyridin-3-yl)oxy]methyl}-4-methylazetidine-1-carboxylate (900 mg, 86.19%) as a yellow solid.

LCMS: [M+H]⁺ found 539.

### 216.5. Synthesis of tert-butyl 2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-4-methylazetidine-1-carboxylate

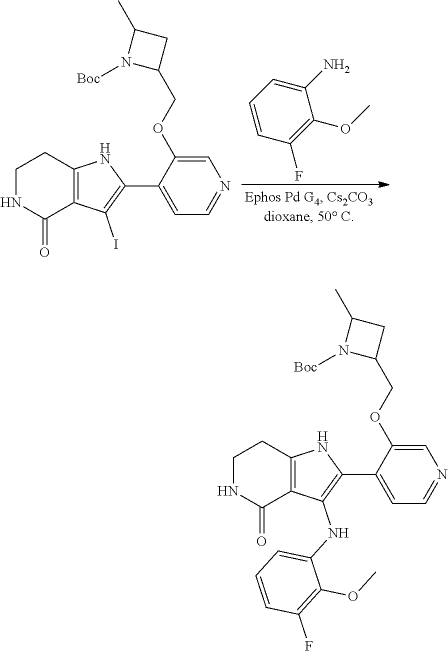

To a stirred mixture of tert-butyl 2-{[(4-{3-iodo-4-oxo-1H,5H,6H,7H-pyrrolo[3,2-c]pyridin-2-yl}pyridin-3-yl)oxy]methyl}-4-methylazetidine-1-carboxylate (900 mg, 1.672 mmol, 1 equiv) and 3-fluoro-2-methoxyaniline (353.92 mg, 2.508 mmol, 1.5 equiv) in dioxane (9 mL) was added Cs₂CO₃ (1089.32 mg, 3.344 mmol, 2 equiv) and Ephos Pd G4 (307.11 mg, 0.334 mmol, 0.2 equiv) in portions at room temperature under nitrogen atmosphere. The resulting mixture was stirred for overnight at 50° C. under nitrogen atmosphere. The aqueous layer was extracted with EtOAc (3×50 mL). The residue was purified by silica gel column chromatography, eluted with CH₂Cl₂/MeOH (20:1) to afford tert-butyl 2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-4-methylazetidine-1-carboxylate (530 mg, 57.48%) as a brown solid.

LCMS: [M+H]⁺ found 552.

### 216.6. Synthesis of 3-((3-fluoro-2-methoxyphenyl)amino)-2-(3-((4-methylazetidin-2-yl)methoxy)pyridin-4-yl)-1,5,6,7-tetrahydro-4H-pyrrolo[3,2-c]pyridin-4-one

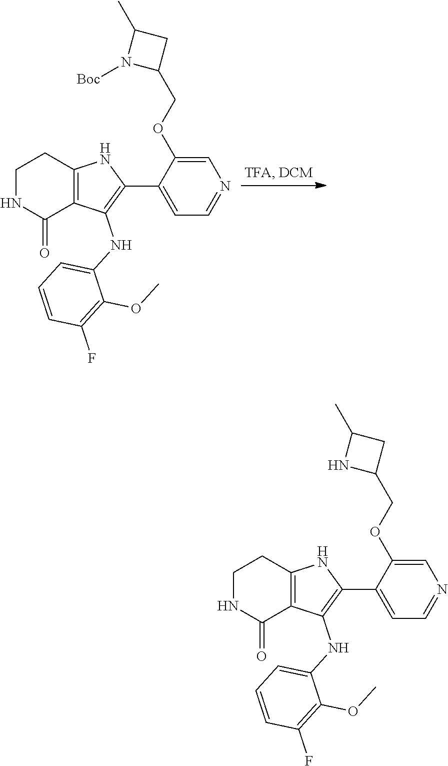 [SMILES: COc1c(F)cccc1Nc1c(-c2ccncc2OCC2CC(C)N2C(=O)OC(C)(C)C)[nH]c2c1C(=O)NCC2 ; COc1c(F)cccc1Nc1c(-c2ccncc2OCC2CC(C)N2)[nH]c2c1C(=O)NCC2]

To a stirred solution of tert-butyl 2-{[(4-{3-[(3-fluoro-2-methoxyphenyl)amino]-4-oxo-1H,5H,6H,7H-pyrrolo[3,2-c]pyridin-2-yl}pyridin-3-yl)oxy]methyl}-4-methylazetidine-1-carboxylate (530 mg, 0.961 mmol, 1 equiv) in DCM (20 mL, 0.135 mmol, 0.14 equiv) was added TFA (4 mL) dropwise at room temperature under nitrogen atmosphere. The resulting mixture was stirred for overnight at room temperature under nitrogen atmosphere. The mixture was basified to pH 9 with saturated NaHCO₃(aq.). The aqueous layer was extracted with DCM (3×50 mL). The crude resulting mixture was used in the next step directly without further purification.

LCMS: [M+H]⁺ found 452.

### 216.7. Synthesis of 3-[(3-fluoro-2-methoxyphenyl)amino]-2-(3-{[4-methyl-1-(prop-2-enoyl)azetidin-2-yl]methoxy}pyridin-4-yl)-1H,5H,6H,7H-pyrrolo[3,2-c]pyridin-4-one

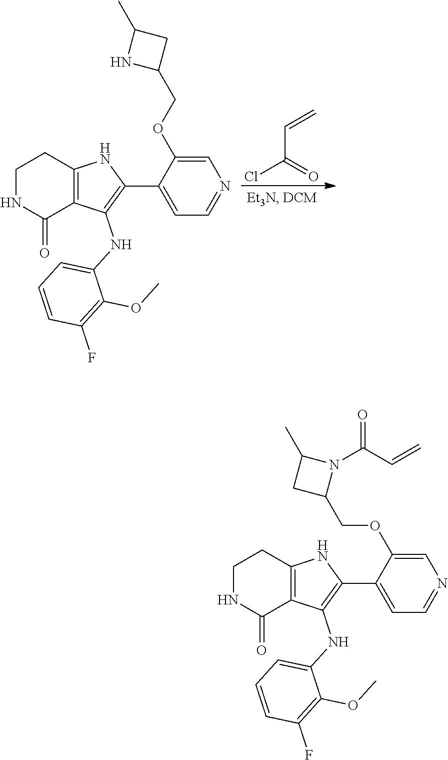

To a stirred mixture of 3-[(3-fluoro-2-methoxyphenyl)amino]-2-{3-[(4-methylazetidin-2-yl)methoxy]pyridin-4-yl}-1H,5H,6H,7H-pyrrolo[3,2-c]pyridin-4-one (400 mg, 0.886 mmol, 1 equiv) and Et3N (268.95 mg, 2.658 mmol, 3 equiv) in DCM (4 mL) was added acryloyl chloride (80.19 mg, 0.886 mmol, 1 equiv) dropwise at 0° C. under nitrogen atmosphere. The resulting mixture was stirred for 10 min at room temperature under nitrogen atmosphere. The aqueous layer was extracted with EtOAc (3×50 mL). The crude product (500 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 14% B to 30% B in 10 min, 30% B; Wave Length: 254/220 nm; RT1(min): 10; Number Of Runs: 0) to afford 3-[(3-fluoro-2-methoxyphenyl)amino]-2-(3-{[4-methyl-1-(prop-2-enoyl)azetidin-2-yl]methoxy}pyridin-4-yl)-1H,5H,6H,7H-pyrrolo[3,2-c]pyridin-4-one (160 mg, 35.72%) as a yellow oil.

LCMS: [M+H]+ found 506.

### 216.8. Synthesis of 3-[(3-fluoro-2-methoxyphenyl)amino]-2-(3-{[(2R*,4R**)-4-methyl-1-(prop-2-enoyl)azetidin-2-yl]methoxy}pyridin-4-yl)-1H,5H,6H,7H-pyrrolo[3,2-c]pyridin-4-one

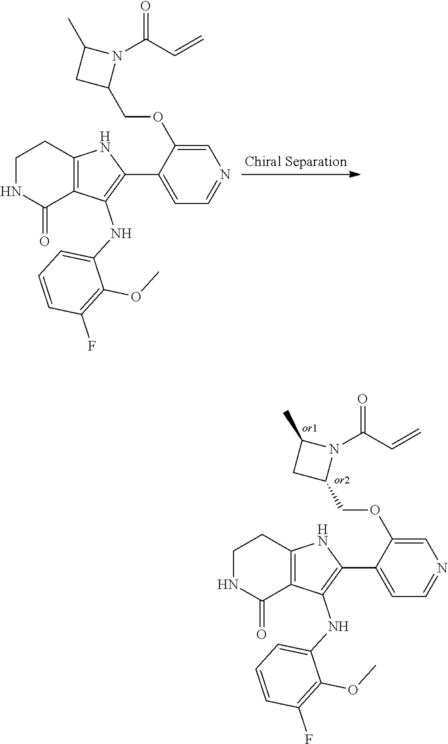 [SMILES: C=CC(=O)N1C(C)CC1COc1cnccc1-c1[nH]c2c(c1Nc1cccc(F)c1OC)C(=O)NCC2 ; C=CC(=O)N1[C@H](COc2cnccc2-c2[nH]c3c(c2Nc2cccc(F)c2OC)C(=O)NCC3)C[C@H]1C]

The crude product (160 mg) was purified by Prep-HPLC with the following conditions (Column: CHIRAL ART Amylose-SA, 2*25 cm, 5 μm; Mobile Phase A: MtBE (0.5% 2M NH₃-MeOH)—HPLC, Mobile Phase B: EtOH—HPLC; Flow rate: 20 mL/min; Gradient: 30% B to 30% B in 17 min; Wave Length: 220/254 nm; RT1(min): 8.569; RT2(min): 14.522; Sample Solvent: EtOH—HPLC; Injection Volume: 2 mL; Number Of Runs: 3) to afford 3-[(3-fluoro-2-methoxyphenyl)amino]-2-(3-{[(2R*,4R**)-4-methyl-1-(prop-2-enoyl)azetidin-2-yl]methoxy}pyridin-4-yl)-1H,5H,6H,7H-pyrrolo[3,2-c]pyridin-4-one (6.7 mg, peak 1) as a white solid.

LCMS: [M+H]⁺ found 506.

¹H NMR (300 MHz, Chloroform-d) δ 11.98 (s, 1H), 8.23 (s, 1H), 7.99-7.82 (m, 2H), 7.50 (d, J=5.8 Hz, 1H), 6.69-6.37 (m, 3H), 6.34-6.20 (m, 1H), 6.03 (d, J=8.0 Hz, 1H), 5.91-5.77 (m, 1H), 5.28 (s, 1H), 4.88 (d, J=7.4 Hz, 1H), 4.57 (d, J=7.9 Hz, 1H), 4.43 (d, J=9.7 Hz, 1H), 4.30 (d, J=9.4 Hz, 1H), 4.12 (d, J=1.4 Hz, 3H), 3.68-3.53 (m, 2H), 3.34-3.12 (m, 2H), 2.91-2.77 (m, 1H), 1.78-1.72 (m, 1H), 1.64 (d, J=6.4 Hz, 3H).

### Example 217. 3-[(3-fluoro-2-methoxyphenyl)amino]-2-(3-{[(2R*,4R**)-4-methyl-1-(prop-2-enoyl)azetidin-2-yl]methoxy}pyridin-4-yl)-1H,5H,6H,7H-pyrrolo[3,2-c]pyridin-4-one (compound 332)

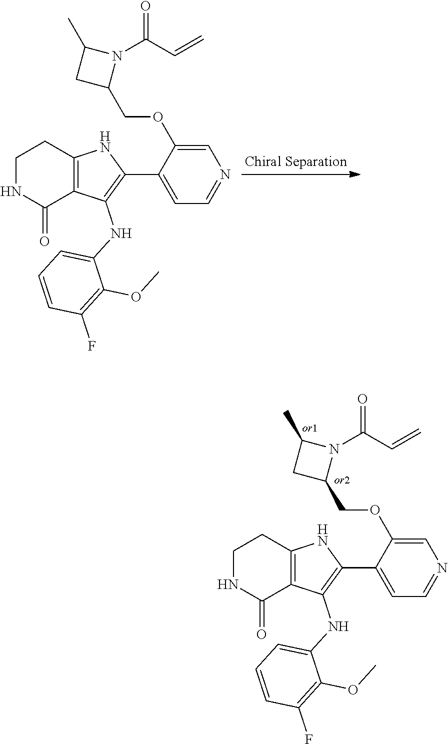 [SMILES: C=CC(=O)N1C(C)CC1COc1cnccc1-c1[nH]c2c(c1Nc1cccc(F)c1OC)C(=O)NCC2 ; C=CC(=O)N1[C@@H](COc2cnccc2-c2[nH]c3c(c2Nc2cccc(F)c2OC)C(=O)NCC3)C[C@H]1C]

The crude product (160 mg) was purified by Prep-HPLC with the following conditions (Column: CHIRAL ART Amylose-SA, 2*25 cm, 5 μm; Mobile Phase A: MtBE (0.5% 2M NH₃-MeOH)—HPLC, Mobile Phase B: EtOH—HPLC; Flow rate: 20 mL/min; Gradient: 30% B to 30% B in 17 min; Wave Length: 220/254 nm; RT1(min): 8.569; RT2(min): 14.522; Sample Solvent: EtOH—HPLC; Injection Volume: 2 mL; Number Of Runs: 3) to afford 3-[(3-fluoro-2-methoxyphenyl)amino]-2-(3-{[(2R*,4R**)-4-methyl-1-(prop-2-enoyl)azetidin-2-yl]methoxy}pyridin-4-yl)-1H,5H,6H,7H-pyrrolo[3,2-c]pyridin-4-one (22.3 mg, peak 2) as a white solid.

LCMS: [M+H]⁺ found 506.

¹H NMR (300 MHz, Chloroform-d) δ 11.88 (s, 1H), 8.21 (s, 1H), 7.99 (d, J=5.2 Hz, 1H), 7.58-7.41 (m, 2H), 6.65-6.52 (m, 1H), 6.49-6.33 (m, 2H), 6.29-6.17 (m, 1H), 6.14-6.02 (m, 1H), 5.81-5.69 (m, 1H), 5.24 (s, 1H), 5.11-4.93 (m, 1H), 4.75-4.57 (m, 1H), 4.44 (t, J=9.7 Hz, 1H), 4.34-4.22 (m, 1H), 4.10 (d, J=1.2 Hz, 3H), 3.68-3.46 (m, 2H), 3.23-3.00 (m, 2H), 2.39-2.24 (m, 1H), 2.21-2.06 (m, 1H), 1.58 (d, J=6.3 Hz, 3H).

### Example 218. rel-3-[(3-chloro-2-methoxyphenyl)amino]-2-(3-{[(1R)-2-(prop-2-enoyl)-2-azaspiro[3.3]heptan-1-yl]methoxy}pyridin-4-yl)-1H,5H,6H,7H-pyrrolo[3,2-c]pyridin-4-one (compound 269)

### 218.1. Synthesis of tert-butyl 1-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-2-azaspiro[3.3]heptane-2-carboxylate

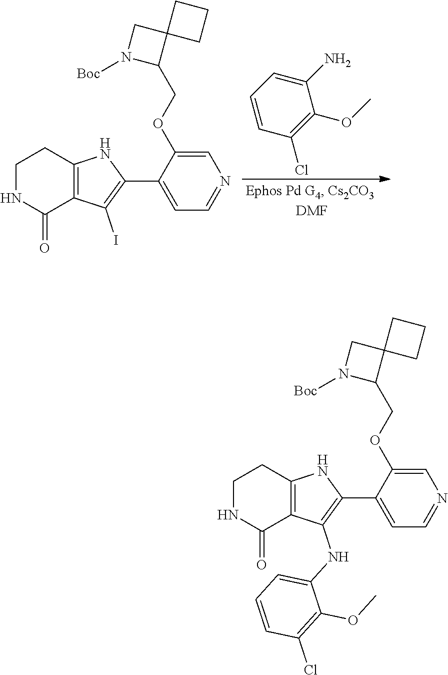

To a stirred solution of tert-butyl 1-{[(4-{3-iodo-4-oxo-1H,5H,6H,7H-pyrrolo[3,2-c]pyridin-2-yl}pyridin-3-yl)oxy]methyl}-2-azaspiro[3.3]heptane-2-carboxylate (380 mg, 0.673 mmol, 1 equiv) and 3-chloro-2-methoxyaniline (212.21 mg, 1.346 mmol, 2 equiv) in DMF (10 mL) were added Cs₂CO₃ (658.08 mg, 2.019 mmol, 3 equiv) and EPhos Pd G4 (123.68 mg, 0.135 mmol, 0.2 equiv) in portions at room temperature under nitrogen atmosphere. The resulting mixture was stirred for overnight at 50° C. under nitrogen atmosphere. The reaction was monitored by LCMS. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by Prep-TLC (PE/EtOAc 1:1) to afford tert-butyl 1-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-2-azaspiro[3.3]heptane-2-carboxylate (120 mg, 30.00%) as a light yellow solid.

LC-MS: (M+H)⁺ found: 594.60.

### 218.2. Synthesis of rac-2-{3-[(1R)-2-azaspiro[3.3]heptan-1-ylmethoxy]pyridin-4-yl}-3-[(3-chloro-2-methoxyphenyl)amino]-1H,5H,6H,7H-pyrrolo[3,2-c]pyridin-4-one

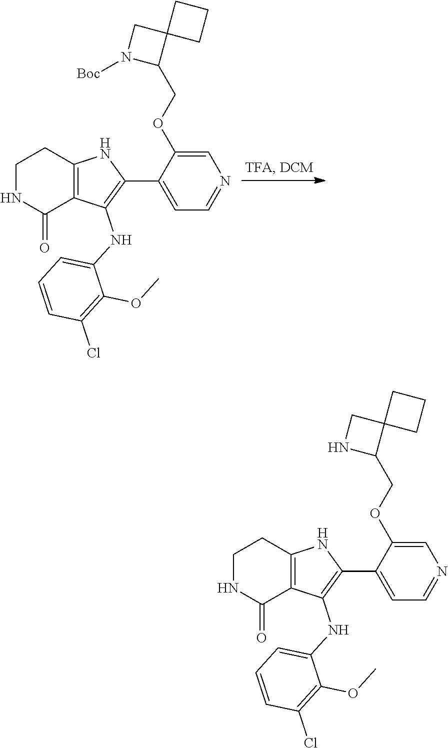 [SMILES: COc1c(Cl)cccc1Nc1c(-c2ccncc2OCC2N(C(=O)OC(C)(C)C)CC23CCC3)[nH]c2c1C(=O)NCC2 ; COc1c(Cl)cccc1Nc1c(-c2ccncc2OCC2NCC23CCC3)[nH]c2c1C(=O)NCC2]

To a stirred solution of rac-tert-butyl (1R)-1-{[(4-{3-[(3-chloro-2-methoxyphenyl)amino]-4-oxo-1H,5H,6H,7H-pyrrolo[3,2-c]pyridin-2-yl}pyridin-3-yl)oxy]methyl}-2-azaspiro[3.3]heptane-2-carboxylate (120 mg, 0.202 mmol, 1 equiv) in DCM (1 mL) was added TFA (3 mL, 40.389 mmol, 199.96 equiv) dropwise at room temperature under nitrogen atmosphere. The resulting mixture was stirred for 1 h at room temperature under nitrogen atmosphere. The reaction was monitored by LCMS. The mixture was basified to pH 10 with saturated NaHCO₃(aq.). The resulting mixture was extracted with CH₂Cl₂ (3×20 mL), dried over anhydrous Na₂SO₄. After filtration, the filtrate was concentrated under reduced pressure. This resulted in rac-2-{3-[(1R)-2-azaspiro[3.3]heptan-1-ylmethoxy]pyridin-4-yl}-3-[(3-chloro-2-methoxyphenyl)amino]-1H,5H,6H,7H-pyrrolo[3,2-c]pyridin-4-one (120 mg, 120.27%) as a brown oil. The crude product was used in the next step directly without further purification.

LC-MS: (M+H)⁺ found 494.50.

### 218.3. Synthesis of rac-3-[(3-chloro-2-methoxyphenyl)amino]-2-(3-{[(1R)-2-(prop-2-enoyl)-2-azaspiro[3.3]heptan-1-yl]methoxy}pyridin-4-yl)-1H,5H,6H,7H-pyrrolo[3,2-c]pyridin-4-one

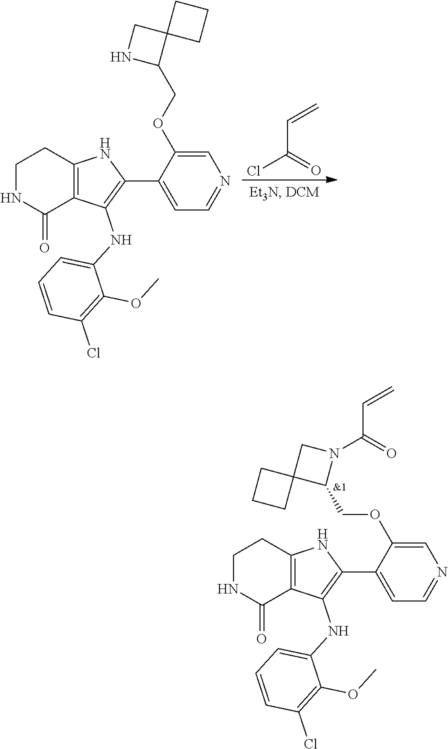

To a stirred solution of rac-2-{3-[(1R)-2-azaspiro[3.3]heptan-1-ylmethoxy]pyridin-4-yl}-3-[(3-chloro-2-methoxyphenyl)amino]-1H,5H,6H,7H-pyrrolo[3,2-c]pyridin-4-one (120 mg, 0.243 mmol, 1 equiv) and Et3N (73.74 mg, 0.729 mmol, 3 equiv) in DCM (1 mL) was added acryloyl chloride (17.59 mg, 0.194 mmol, 0.8 equiv) dropwise at 0° C. under nitrogen atmosphere. The resulting mixture was stirred for 1 h at room temperature under nitrogen atmosphere. The reaction was monitored by LCMS. The resulting mixture was extracted with EtOAc (3×5 mL). The combined organic layers were washed with brine (2×5 mL), dried over anhydrous Na₂SO₄. After filtration, the filtrate was concentrated under reduced pressure. The crude product (120 mg) was purified by Prep-HPLC with the following conditions (Column: XBridge Prep OBD C18 Column, 19*250 mm, 5 μm; Mobile Phase A: Water (10 mmol/L NH₄HCO₃), Mobile Phase B: ACN; Flow rate: 25 mL/min; Gradient: 40% B to 70% B in 7 min, 70% B; Wave Length: 254 nm; RT1(min): 6.93; Number Of Runs: 0) to afford rac-3-[(3-chloro-2-methoxyphenyl)amino]-2-(3-{[(1R)-2-(prop-2-enoyl)-2-azaspiro[3.3]heptan-1-yl]methoxy}pyridin-4-yl)-1H,5H,6H,7H-pyrrolo[3,2-c]pyridin-4-one (120 mg, 90.14%) as a light yellow solid.

LC-MS: (M+H)⁺ found 548.50.

### 218.4. Synthesis of rel-3-[(3-chloro-2-methoxyphenyl)amino]-2-(3-{[(1R)-2-(prop-2-enoyl)-2-azaspiro[3.3]heptan-1-yl]methoxy}pyridin-4-yl)-1H,5H,6H,7H-pyrrolo[3,2-c]pyridin-4-one

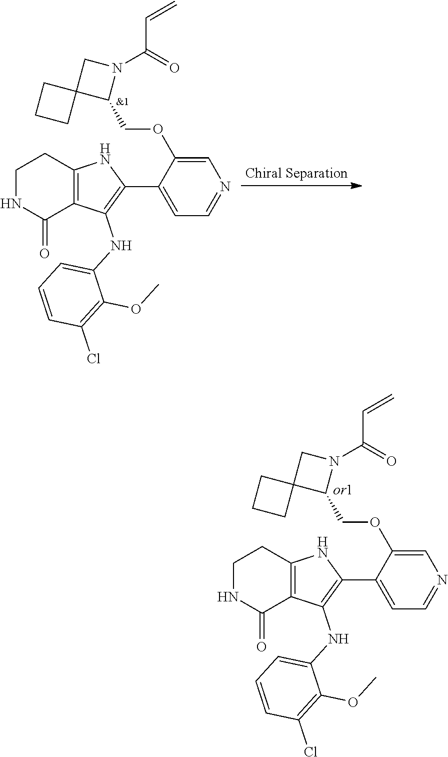 [SMILES: C=CC(=O)N1CC2(CCC2)[C@H]1COc1cnccc1-c1[nH]c2c(c1Nc1cccc(Cl)c1OC)C(=O)NCC2 ; C=CC(=O)N1CC2(CCC2)[C@H]1COc1cnccc1-c1[nH]c2c(c1Nc1cccc(Cl)c1OC)C(=O)NCC2]

The product (50 mg) was purified by Chiral-HPLC with the following conditions (Column: CHIRALPAK IA-3, 4.6*50 mm 3 um; Mobile Phase A: Hex (0.1% DEA):EtOH=50:50; Flow rate: 1 mL/min; Gradient: 0% B to 0% B; Injection Volume: 5 ul mL) to afford rel-3-[(3-chloro-2-methoxyphenyl)amino]-2-(3-{[(1R)-2-(prop-2-enoyl)-2-azaspiro[3.3]heptan-1-yl]methoxy}pyridin-4-yl)-1H,5H,6H,7H-pyrrolo[3,2-c]pyridin-4-one (2 mg, 4.00%, peak 1) as a light yellow solid.

LC-MS: (M+H)⁺ found 548.50.

¹H NMR (300 MHz, Chloroform-d) δ 11.85 (s, 1H), 8.30 (s, 1H), 7.97 (s, 1H), 7.63 (s, 1H), 7.46 (s, 1H), 6.72 (dd, J=8.0, 1.6 Hz, 1H), 6.62 (t, 1H), 6.39 (dd, J=16.8, 2.2 Hz, 1H), 6.32-6.17 (m, 2H), 5.78 (dd, J=10.0, 2.2 Hz, 1H), 5.33 (s, 1H), 4.82-4.71 (m, 1H), 4.43 (d, J=7.5 Hz, 2H), 4.36 (d, J=8.5 Hz, 1H), 4.25 (d, J=8.5 Hz, 1H), 4.08 (s, 3H), 3.65-3.55 (m, 2H), 3.26-3.06 (m, 2H), 2.44-2.22 (m, 3H), 2.17-2.07 (m, 1H), 2.01-1.94 (m, 2H).

### Example 219. rel-3-[(3-chloro-2-methoxyphenyl)amino]-2-(3-{[(1R)-2-(prop-2-enoyl)-2-azaspiro[3.3]heptan-1-yl]methoxy}pyridin-4-yl)-1H,5H,6H,7H-pyrrolo[3,2-c]pyridin-4-one (compound 268)

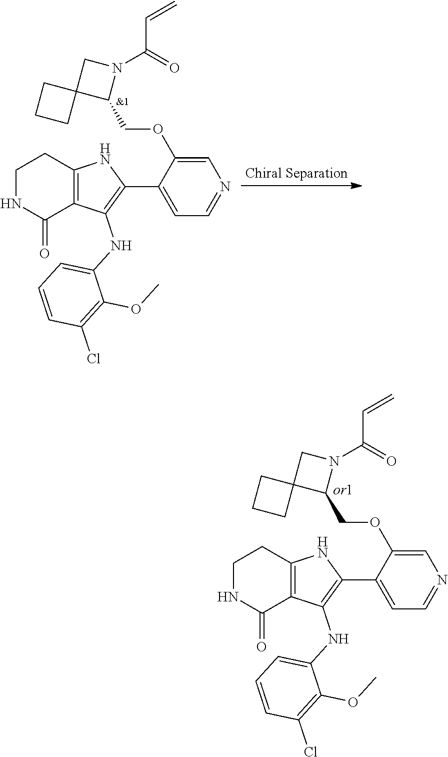 [SMILES: C=CC(=O)N1CC2(CCC2)[C@H]1COc1cnccc1-c1[nH]c2c(c1Nc1cccc(Cl)c1OC)C(=O)NCC2 ; C=CC(=O)N1CC2(CCC2)[C@@H]1COc1cnccc1-c1[nH]c2c(c1Nc1cccc(Cl)c1OC)C(=O)NCC2]

The product (50 mg) was purified by Chiral-HPLC with the following conditions (Column: CHIRALPAK IA-3, 4.6*50 mm 3 um; Mobile Phase A: Hex (0.1% DEA):EtOH=50:50; Flow rate: 1 mL/min; Gradient: 0% B to 0% B; Injection Volume: 5 ul mL) to afford rel-3-[(3-chloro-2-methoxyphenyl)amino]-2-(3-{[(1R)-2-(prop-2-enoyl)-2-azaspiro[3.3]heptan-1-yl]methoxy}pyridin-4-yl)-1H,5H,6H,7H-pyrrolo[3,2-c]pyridin-4-one (2.1 mg, 4.20%, peak 2) as a light yellow solid.

LC-MS: (M+H)⁺ found 548.20.

¹H NMR (300 MHz, Chloroform-d) δ 11.86 (s, 1H), 8.28 (s, 1H), 7.96 (s, 1H), 7.64 (s, 1H), 7.45 (s, 1H), 6.73 (dd, J=8.1, 1.6 Hz, 1H), 6.62 (t, 1H), 6.39 (dd, J=16.8, 2.2 Hz, 1H), 6.31-6.19 (m, 2H), 5.78 (dd, J=9.9, 2.2 Hz, 1H), 5.31 (s, 1H), 4.82-4.69 (m, 1H), 4.43 (d, J=7.6 Hz, 2H), 4.36 (d, J=8.7 Hz, 1H), 4.25 (d, J=8.6 Hz, 1H), 4.08 (s, 3H), 3.65-3.55 (m, 2H), 3.22-3.10 (m, 2H), 2.45-2.26 (m, 3H), 2.18-2.06 (m, 1H), 2.01-1.94 (m, 2H).

### Example 220. 3-((3-chloro-2-methoxyphenyl)amino)-2-(3-(2-(pyridin-2-yl)ethoxy)pyridin-4-yl)-1,5,6,7-tetrahydro-4H-pyrrolo[3,2-c]pyridin-4-one (compound 473)

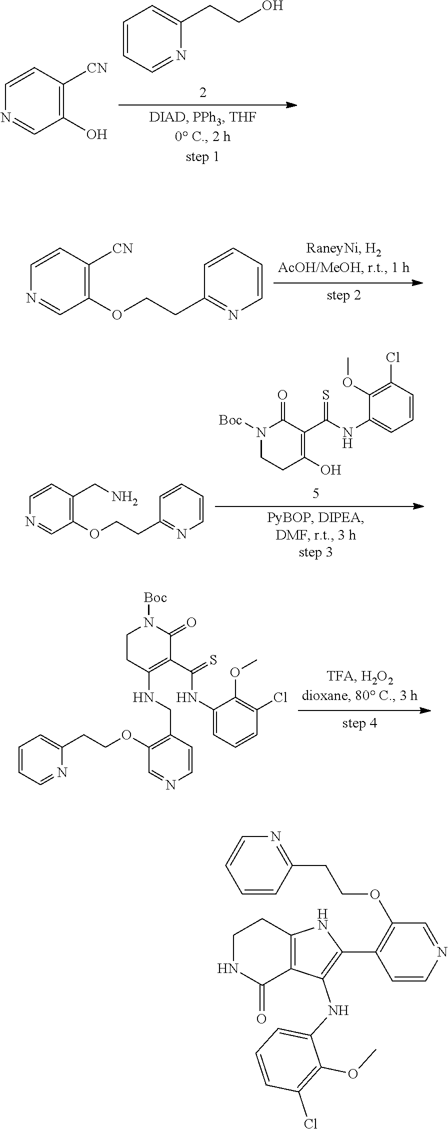

### 220-Step 1

To a stirred mixture of 2-(pyridin-2-yl)ethan-1-ol (1 g, 8.1 mmol), 3-hydroxyisonicotinonitrile (972 mg, 8.1 mmol), and PPh₃ (2.55 g, 9.72 mmol) in THF (20 mL) was added DIAD (1.96 g, 9.72 mmol) at 0° C. The reaction mixture was stirred at 0° C. for 2 h under N₂. After completion, the reaction was quenched with water (50 mL), extracted with EtOAc (50 mL×3). The combined organic layers were washed with brine (40 mL), dried over Na₂SO₄, filtered and concentrated in vacuo to give the crude, which was purified with silica gel column chromatography (eluent: PE/EtOAc from 0 to 40%) to give 3-(2-(pyridin-2-yl)ethoxy)isonicotinonitrile (200 mg, yield: 11%) as a yellow solid.

MS (ESI): mass calcd. for C₁₃H₁₁N₃O, 225.09, m/z found 226.1 [M+H]⁺.

### 220-Step 2

A mixture of 3-(2-(pyridin-2-yl)ethoxy)isonicotinonitrile (200 mg, 0.89 mmol) and Raney-Ni (50 mg) in MeOH (20 mL)/AcOH (5 mL) was stirred for 1 h at room temperature under hydrogen atmosphere. After completion, the mixture was filtered, washed with MeOH (40 mL). The filtrate was concentrated under reduced pressure to give the crude, which was purified by Prep-TLC (eluent: DCM/MeOH/NH4OH=10/1/0.1) to afford (3-(2-(pyridin-2-yl)ethoxy)pyridin-4-yl)methanamine (150 mg, 74%) as a yellow solid.

MS (ESI): mass calcd. for C₁₃H₁₅N₃O, 229.12, m/z found 230.2 [M+H]⁺.

### 220-Step 3

A mixture of (3-(2-(pyridin-2-yl)ethoxy)pyridin-4-yl)methanamine (140 mg, 0.61 mmol), tert-butyl 5-((3-chloro-2-methoxyphenyl)carbamothioyl)-4-hydroxy-6-oxo-3,6-dihydropyridine-1(2H)-carboxylate (252 mg, 0.61 mmol), PyBOP (380 mg, 0.73 mmol) and DIPEA (236 mg, 1.83 mmol) in DMF (10 mL) was stirred at room temperature for 3 h. After completion, the resulting mixture was diluted with water (30 mL), extracted with EtOAc (30 mL×3). The combined organic layers were washed with brine (30 mL), dried over anhydrous Na₂SO₄. After filtration, the filtrate was concentrated under reduced pressure. The residue was purified by Prep-TLC (DCM/MeOH=15:1) to afford tert-butyl 5-((3-chloro-2-methoxyphenyl)carbamothioyl)-6-oxo-4-(((3-(2-(pyridin-2-yl)ethoxy)pyridin-4-yl)methyl)amino)-3,6-dihydropyridine-1(2H)-carboxylate (140 mg, 37%) as a yellow solid.

MS (ESI): mass calcd. for C₃₁H₃₄ClN₅O₅S, 623.20, m/z found 624.2 [M+H]⁺.

### 220-Step 4

To a solution of tert-butyl 5-((3-chloro-2-methoxyphenyl)carbamothioyl)-6-oxo-4-(((3-(2-(pyridin-2-yl)ethoxy)pyridin-4-yl)methyl)amino)-3,6-dihydropyridine-1(2H)-carboxylate (140 mg, 0.22 mmol) in 1,4-dioxane (5 mL) was added TFA (75 mg, 0.66 mmol) and H₂O₂ (74.8 mg, 0.66 mmol, 30% solution in H₂O). The resulting mixture was stirred at 80° C. for 3h. The reaction mixture was concentrated to give the crude product, which was purified by Prep-TLC (DCM/MeOH=15/1) to give 3-((3-chloro-2-methoxyphenyl)amino)-2-(3-(2-(pyridin-2-yl)ethoxy)pyridin-4-yl)-1,5,6,7-tetrahydro-4H-pyrrolo[3,2-c]pyridin-4-one (20 mg, yield: 18.5%) as a yellow solid.

MS (ESI): mass calcd. for C₂₆H₂₄ClN₅O₃, 489.16, m/z found 490.0 [M+H]⁺.

¹H NMR (400 MHz, DMSO-d6) δ 11.86 (s, 1H), 8.57 (d, J=4.9 Hz, 1H), 8.35 (s, 1H), 7.98 (d, J=5.1 Hz, 1H), 7.81-7.77 (m, 1H), 7.53 (s, 1H), 7.44 (d, J=7.8 Hz, 1H), 7.32-7.29 (m, 2H), 7.15 (s, 1H), 6.68-6.64 (m, 2H), 6.09 (dd, J=5.9, 3.8 Hz, 1H), 4.52-4.49 (m, 2H), 3.89 (s, 3H), 3.46-3.42 (m, 2H), 3.31-3.28 (m, 2H), 2.93 (t, J=6.8 Hz, 2H).

### Example 221. 3-((3-chloro-2-methoxyphenyl)amino)-2-(3-((6-fluoropyridin-2-yl)methoxy)pyridin-4-yl)-1,5,6,7-tetrahydro-4H-pyrrolo[3,2-c]pyridin-4-one (compound 529)

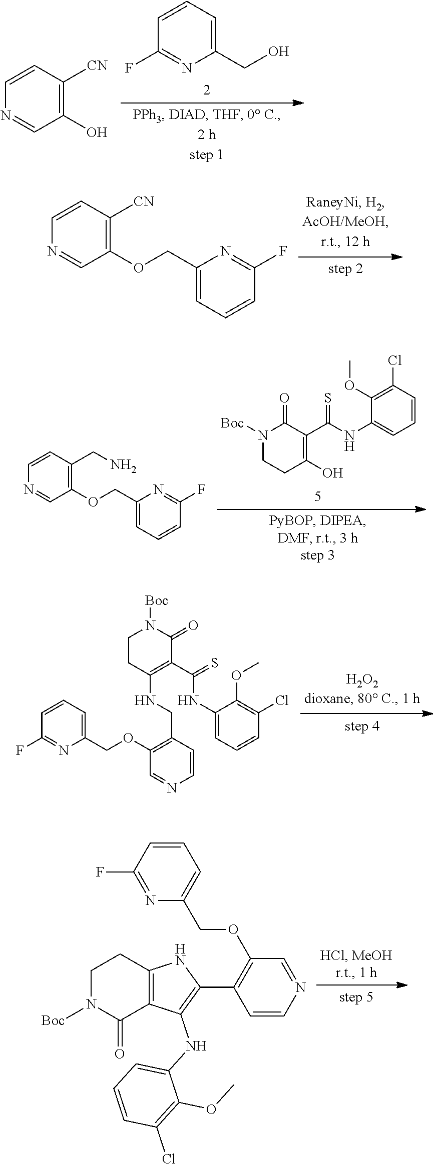

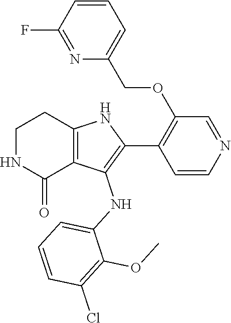 [SMILES: COc1c(Cl)cccc1Nc1c(-c2ccncc2OCc2cccc(F)n2)[nH]c2c1C(=O)NCC2]

### 221-Step 1

To a mixture of 3-hydroxyisonicotinonitrile (354 mg, 2.95 mmol), (6-fluoropyridin-2-yl)methanol (340 mg, 2.68 mmol), and PPh₃ (844 mg, 3.22 mmol) in THF (35 mL) was added DIAD (651 mg, 3.22 mmol) at 0° C. The reaction mixture was stirred at 0° C. for 2 h under N₂. After completion, the reaction was quenched with H₂O (50 mL), extracted with EtOAc (50 mL×3). The combined organic layers were washed with brine (40 mL), dried over Na₂SO₄, concentrated in vacuo to give the crude, which was purified with Prep-TLC (PE/EA=2/1) to give the 3-((6-fluoropyridin-2-yl)methoxy)isonicotinonitrile (300 mg, yield: 49%) as a yellow solid.

MS (ESI): mass calcd. for C₁₂H₈FN₃O, 229.07, m/z found 230.0 [M+H]⁺.

### 221-Step 2

A mixture of 3-((6-fluoropyridin-2-yl)methoxy)isonicotinonitrile (300 mg, 1.31 mmol) and Raney-Ni (500 mg) in MeOH (20 mL) and AcOH (5 mL) was stirred for 1 h at room temperature under hydrogen atmosphere. The reaction mixture was filtered, washed with MeOH (30 mL). The filtrate was concentrated under reduced pressure to give the crude, which was purified by Prep-TLC (eluent: DCM/MeOH/NH₄OH=10/1/0.5) to afford (3-((6-fluoropyridin-2-yl)methoxy)pyridin-4-yl)methanamine (150 mg, 49%) as a yellow solid.

MS (ESI): mass calcd. for C₁₂H₁₂FN₃O, 233.10, m/z found 234.1 [M+H]⁺.

### 221-Step 3

A mixture of tert-butyl 5-((3-chloro-2-methoxyphenyl)carbamothioyl)-4-hydroxy-6-oxo-3,6-dihydropyridine-1(2H)-carboxylate (264 mg, 0.64 mmol), (3-((6-fluoropyridin-2-yl)methoxy)pyridin-4-yl)methanamine (150 mg, 0.64 mmol), PyBOP (400 mg, 0.77 mmol) and DIEA (248 mg, 1.92 mmol) in DMF (10 mL) was stirred for 3 h at room temperature. The resulting mixture was diluted with water (100 mL), extracted with EtOAc (30 mL×3). The combined organic layers were washed with brine (30 mL), dried over anhydrous Na₂SO₄. After filtration, the filtrate was concentrated under reduced pressure. The residue was purified by Prep-TLC (DCM/MeOH=15:1) to afford tert-butyl 5-((3-chloro-2-methoxyphenyl)carbamothioyl)-4-(((3-((6-fluoropyridin-2-yl)methoxy)pyridin-4-yl)methyl)amino)-6-oxo-3,6-dihydropyridine-1(2H)-carboxylate (100 mg, 25%) as a yellow solid.

MS (ESI): mass calcd. for C₃₀H₃₁ClFN₅O₅S, 627.17, m/z found 628.1 [M+H]⁺.

### 221-Step 4

To a solution of tert-butyl 5-((3-chloro-2-methoxyphenyl)carbamothioyl)-4-(((3-((6-fluoropyridin-2-yl)methoxy)pyridin-4-yl)methyl)amino)-6-oxo-3,6-dihydropyridine-1(2H)-carboxylate (100 mg 0.16 mmol) in dioxane (5 mL) was added H₂O₂ (30% in H₂O, 0.2 mL). The reaction was stirred at 80° C. for 1 h. The reaction was concentrated in vacuo to give the crude, which was purified with Prep-TLC (DCM/MeOH=15/1) to give the tert-butyl 3-((3-chloro-2-methoxyphenyl)amino)-2-(3-((6-fluoropyridin-2-yl)methoxy)pyridin-4-yl)-4-oxo-1,4,6,7-tetrahydro-5H-pyrrolo[3,2-c]pyridine-5-carboxylate (50 mg, yield: 52.6%) as a yellow solid.

MS (ESI): mass calcd. for C₃₀H₂₉ClFN₅O₅, 593.18, m/z found 594.2 [M+H]⁺.

### 221-Step 5

To a solution of tert-butyl 3-((3-chloro-2-methoxyphenyl)amino)-2-(3-((6-fluoropyridin-2-yl)methoxy)pyridin-4-yl)-4-oxo-1,4,6,7-tetrahydro-5H-pyrrolo[3,2-c]pyridine-5-carboxylate (50 mg, 0.084 mmol) in DCM (5 mL) was added HCl (4M solution in dioxane, 0.5 mL). The resulting mixture was stirred at room temperature for 1 h. The reaction mixture was concentrated to give the crude product, which was purified by Prep-TLC (DCM/MeOH=15/1) to give 3-((3-chloro-2-methoxyphenyl)amino)-2-(3-((6-fluoropyridin-2-yl)methoxy)pyridin-4-yl)-1,5,6,7-tetrahydro-4H-pyrrolo[3,2-c]pyridin-4-one (25 mg, yield: 61%) as a yellow solid.

MS (ESI): mass calcd. for C₂₅H₂₁ClFN₅O₃, 493.13, m/z found 494.2 [M+H]⁺.

¹H NMR (400 MHz, DMSO) δ 11.54 (s, 1H), 8.39 (s, 1H), 8.13-8.00 (m, 2H), 7.53 (s, 1H), 7.41 (dd, J=7.4, 2.0 Hz, 1H), 7.34 (d, J=5.0 Hz, 1H), 7.23-7.11 (m, 2H), 6.71-6.62 (m, 2H), 6.16 (dd, J=6.8, 2.8 Hz, 1H), 5.45 (s, 2H), 3.85 (s, 3H), 3.53-3.37 (m, 2H), 2.92 (t, J=6.8 Hz, 2H).

### Example 222. 3-((3-chloro-2-methoxyphenyl)amino)-2-(3-((3-fluoropyridin-2-yl)methoxy)pyridin-4-yl)-1,5,6,7-tetrahydro-4H-pyrrolo[3,2-c]pyridin-4-one (compound 530)

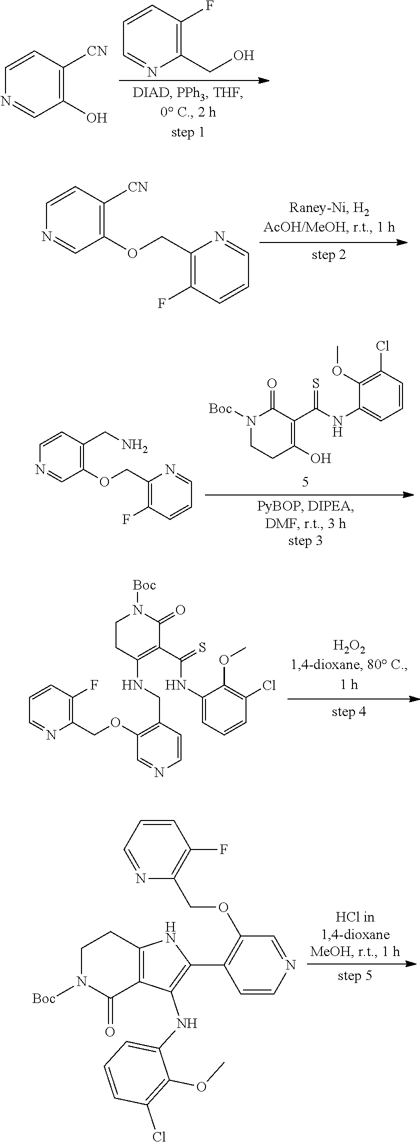

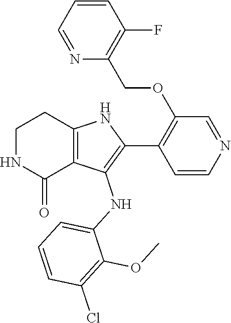 [SMILES: COc1c(Cl)cccc1Nc1c(-c2ccncc2OCc2ncccc2F)[nH]c2c1C(=O)NCC2]

### 222-Step 1

To a mixture of 3-hydroxyisonicotinonitrile (600 mg, 5.0 mmol), (3-fluoropyridin-2-yl)methanol (635 mg, 5.0 mmol) and PPh₃ (2.63 g, 10.0 mmol) in THF (25 mL) was added DIAD (2.02 g, 10.0 mmol) at 0° C. The resulting reaction mixture was stirred at 0 20° C. for 2 h under N₂. After completion, the reaction was quenched with H₂O (40 mL), extracted with EtOAc (40 mL×3). The combined organic layers were washed with brine (30 mL), dried over Na₂SO₄, filtered and concentrated in vacuo to give the crude, which was purified by silica gel column chromatography (DCM/MeOH=1/20) to give 3-((3-fluoropyridin-2-yl)methoxy)isonicotinonitrile (420 mg, yield: 36.7%) as a yellow solid.

MS (ESI): mass calcd. for C₁₂H₈FN₃O, 229.07, m/z found 230.1 [M+H]⁺.

### 222-Step 2

A mixture of 3-((3-fluoropyridin-2-yl)methoxy)isonicotinonitrile (420 mg, 1.83 mmol) and Raney-Ni (100 mg) in MeOH (20 mL) and AcOH (5 mL) was stirred at room temperature for 1 h under hydrogen atmosphere. After completion, the reaction mixture was filtered through celite and the filter cake was washed with MeOH (20 mL). The filtrate was concentrated in vacuo to give the crude, which was purified by Prep-TLC (eluent: DCM/MeOH/NH₄OH=10/1/0.1) to afford (3-((3-fluoropyridin-2-yl)methoxy)pyridin-4-yl)methanamine (310 mg, yield: 72.7%) as a yellow solid.

MS (ESI): mass calcd. for C₁₂H₁₂FN₃O, 233.10, m/z found 234.2 [M+H]⁺.

### 222-Step 3

A mixture of tert-butyl 5-((3-chloro-2-methoxyphenyl)carbamothioyl)-4-hydroxy-6-oxo-3,6-dihydropyridine-1(2H)-carboxylate (354 mg, 0.86 mmol), (3-((3-fluoropyridin-2-yl)methoxy)pyridin-4-yl)methanamine (240 mg, 1.03 mmol), PyBOP (536 mg, 1.03 mmol) and DIPEA (333 mg, 2.58 mmol) in DMF (15 mL) was stirred at room temperature for 3 h under N₂. The resulting mixture was diluted with water (50 mL) and extracted with EtOAc (50 mL×3). The combined organic layers were washed with brine (30 mL), dried over anhydrous Na₂SO₄, filtered and concentrated in vacuo to give the crude, which was purified by Prep-TLC (DCM/MeOH=30/1) to afford tert-butyl 5-((3-chloro-2-methoxyphenyl)carbamothioyl)-4-(((3-((3-fluoropyridin-2-yl)methoxy)pyridin-4-yl)methyl)amino)-6-oxo-3,6-dihydropyridine-1(2H)-carboxylate (120 mg, yield: 22%) as a yellow solid.

MS (ESI): mass calcd. for C₃₀H₃₁ClFN₅O₅S, 627.17, m/z found 628.2 [M+H]⁺.

### 222-Step 4

To a solution of tert-butyl 5-((3-chloro-2-methoxyphenyl)carbamothioyl)-4-(((3-((3-fluoropyridin-2-yl)methoxy)pyridin-4-yl)methyl)amino)-6-oxo-3,6-dihydropyridine-1(2H)-carboxylate (120 mg, 0.19 mmol) in 1,4-dioxane (5 mL) was added H₂O₂ (30% solution in H₂O, 64.5 mg, 0.57 mmol). The resulting reaction was stirred at 80° C. for 1 h. The reaction was concentrated in vacuo to give the crude, which was purified with Prep-TLC (DCM/MeOH=20/1) to give the tert-butyl 3-((3-chloro-2-methoxyphenyl)amino)-2-(3-((3-fluoropyridin-2-yl)methoxy)pyridin-4-yl)-4-oxo-1,4,6,7-tetrahydro-5H-pyrrolo[3,2-c]pyridine-5-carboxylate (60 mg, yield: 53%) as a yellow solid.

MS (ESI): mass calcd. for C₃₀H₂₉ClFN₅O₅, 593.18, m/z found 594.2 [M+H]⁺.

### 222-Step 5

To a solution of tert-butyl 3-((3-chloro-2-methoxyphenyl)amino)-2-(3-((3-fluoropyridin-2-yl)methoxy)pyridin-4-yl)-4-oxo-1,4,6,7-tetrahydro-5H-pyrrolo[3,2-c]pyridine-5-carboxylate (60 mg, 0.10 mmol) in MeOH (5 mL) was added HCl (4M solution in 1,4-dioxane, 0.5 mL). The resulting mixture was stirred at room temperature for 1 h. The reaction mixture was concentrated to give the crude product, which was purified by Prep-TLC (DCM/MeOH=15/1) to give 3-((3-chloro-2-methoxyphenyl)amino)-2-(3-((3-fluoropyridin-2-yl)methoxy)pyridin-4-yl)-1,5,6,7-tetrahydro-4H-pyrrolo[3,2-c]pyridin-4-one (35 mg, yield: 71%) as a yellow solid.

MS (ESI): mass calcd. for C₂₅H₂₁ClFN₅O₃, 493.13, m/z found 494.1 [M+H]⁺.

¹H NMR (400 MHz, DMSO) δ 12.28 (s, 1H), 8.60 (d, J=4.7 Hz, 1H), 8.56 (s, 1H), 8.04 (d, J=5.1 Hz, 1H), 7.89 (t, J=9.3 Hz, 1H), 7.61-7.57 (m, 1H), 7.56 (s, 1H), 7.36 (d, J=5.1 Hz, 1H), 7.18 (s, 1H), 6.71-6.67 (m, 2H), 6.16 (dd, J=6.8, 2.8 Hz, 1H), 5.69 (s, 2H), 3.90 (s, 3H), 3.47-3.43 (m, 2H), 2.98 (t, J=6.8 Hz, 2H).

### Example 223. 3-((3-chloro-2-methoxyphenyl)amino)-2-(3-((5-fluoropyridin-2-yl)methoxy)pyridin-4-yl)-1,5,6,7-tetrahydro-4H-pyrrolo[3,2-c]pyridin-4-one (compound 531)

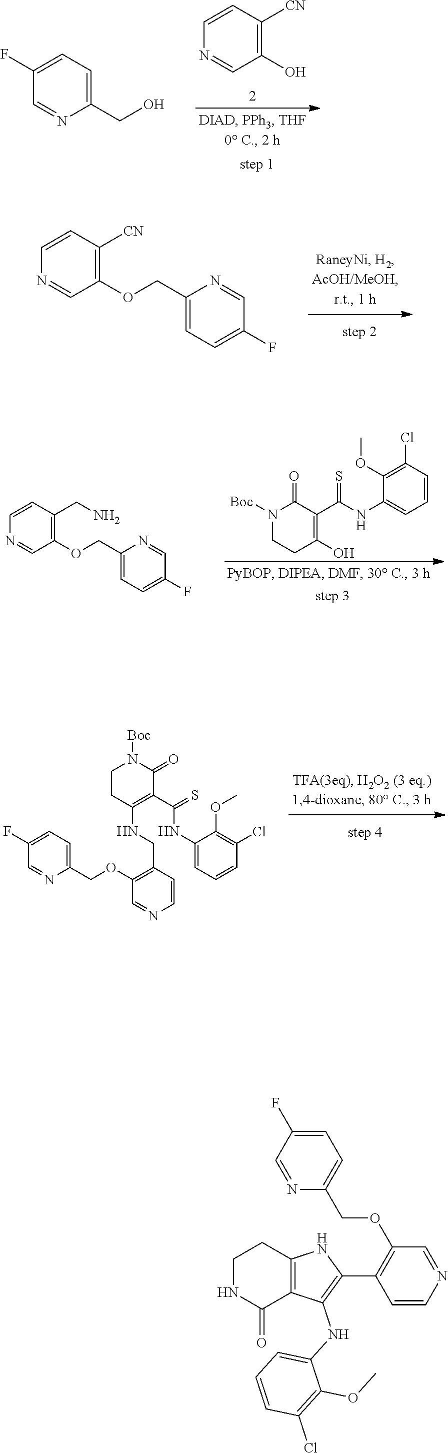

### 223-Step 1

To a mixture of (5-fluoropyridin-2-yl)methanol (700 mg, 5.5 mmol), 3-hydroxyisonicotinonitrile (660 mg, 5.5 mmol) and PPh₃ (1.74 g, 6.6 mmol) in THF (30 mL) was added DIAD (1.33 g, 6.6 mmol) at 0° C. The reaction mixture was stirred at 0° C. for 2 h under N₂. After completion, the reaction was quenched with H₂O (30 mL), extracted with EtOAc (50 mL×3). The combined organic layers were washed with brine (40 mL), dried over Na₂SO₄, concentrated in vacuo to give the crude, which was purified with Prep-TLC (PE/EA=2/1) to give the 3-((5-fluoropyridin-2-yl)methoxy)isonicotinonitrile (480 mg, yield: 38%) as a yellow solid.

MS (ESI): mass calcd. for C₁₂H₈FN₃O, 229.07, m/z found 230.0 [M+H]⁺.

### 223-Step 2

A mixture of 3-((5-fluoropyridin-2-yl)methoxy)isonicotinonitrile (480 mg, 2.10 mmol) and Raney-Ni (50 mg) in MeOH (20 mL) and AcOH (4 mL) was stirred for 1 h at room temperature under hydrogen atmosphere. The reaction mixture was filtered through celite and the filter cake was washed with MeOH (20 mL). The filtrate was concentrated in vacuo to give the crude, which was purified by Prep-TLC (eluent: DCM/MeOH/NH₄OH=10/1/0.1) to afford (3-((5-fluoropyridin-2-yl)methoxy)pyridin-4-yl)methanamine (410 mg, yield: 84%) as a yellow solid.

MS (ESI): mass calcd. for C₁₂H₁₂FN₃O, 233.10, m/z found 234.1 [M+H]⁺.

### 223-Step 3

A mixture of (3-((5-fluoropyridin-2-yl)methoxy)pyridin-4-yl)methanamine (410 mg, 1.76 mmol), tert-butyl 5-((3-chloro-2-methoxyphenyl)carbamothioyl)-4-hydroxy-6-oxo-3,6-dihydropyridine-1(2H)-carboxylate (723 mg, 1.76 mmol), PyBOP (1.1 g, 2.1 mmol) and DIPEA (677 mg, 5.25 mmol) in DMF (15 mL) was stirred at 30° C. for 3 h. After completion, the reaction was diluted with water (40 mL) and extracted with EtOAc (40 mL×3). The combined organic layers were washed with brine (40 mL), dried over anhydrous Na₂SO₄, filtered and concentrated in vacuo to give the crude, which was purified by silica gel column chromatography (eluent: DCM/MeOH=1/20) to afford tert-butyl 5-((3-chloro-2-methoxyphenyl)carbamothioyl)-4-(((3-((5-fluoropyridin-2-yl)methoxy)pyridin-4-yl)methyl)amino)-6-oxo-3,6-dihydropyridine-1(2H)-carboxylate (330 mg, yield: 30%) as a yellow solid.

MS (ESI): mass calcd. for C₃₀H₃₁ClFN₅O₅S, 627.17, m/z found 628.2 [M+H]⁺.

### 223-Step 4

To a solution of tert-butyl 5-((3-chloro-2-methoxyphenyl)carbamothioyl)-4-(((3-((5-fluoropyridin-2-yl)methoxy)pyridin-4-yl)methyl)amino)-6-oxo-3,6-dihydropyridine-1(2H)-carboxylate (145 mg, 0.23 mmol) in 1,4-dioxane (5 mL) was added TFA (79 mg, 0.69 mmol) and H₂O₂ (30% solution in H₂O, 0.2 mL, 0.69 mmol). The resulting mixture was stirred at 80° C. for 3 h. The reaction mixture was concentrated to give the crude product, which was purified by Prep-TLC (DCM/MeOH=15/1) to give 3-((3-chloro-2-methoxyphenyl)amino)-2-(3-((5-fluoropyridin-2-yl)methoxy)pyridin-4-yl)-1,5,6,7-tetrahydro-4H-pyrrolo[3,2-c]pyridin-4-one (29 mg, yield: 25.6%) as a yellow solid.

MS (ESI): mass calcd. for C₂₅H₂₁ClFN₅O₃, 493.13, m/z found 494.7 [M+H]⁺.

¹H NMR (400 MHz, DMSO) δ 11.87 (s, 1H), 8.69 (d, J=2.8 Hz, 1H), 8.42 (s, 1H), 8.04 (d, J=5.1 Hz, 1H), 7.87-7.82 (m, 1H), 7.59 (dd, J=8.7, 4.5 Hz, 1H), 7.53 (s, 1H), 7.33 (d, J=5.1 Hz, 1H), 7.15 (s, 1H), 6.68-6.65 (m, 2H), 6.16 (dd, J=6.4, 3.3 Hz, 1H), 5.50 (s, 2H), 3.86 (s, 3H), 3.46-3.43 (m, 2H), 2.95 (t, J=6.8 Hz, 2H).

### Example 224. (S)-3-((3-chloro-2-methoxyphenyl)amino)-2-(3-(1-(pyridin-2-yl)ethoxy)pyridin-4-yl)-1, 5, 6, 7-tetrahydro-4H-pyrrolo[3, 2-c]pyridin-4-one (compound 532)

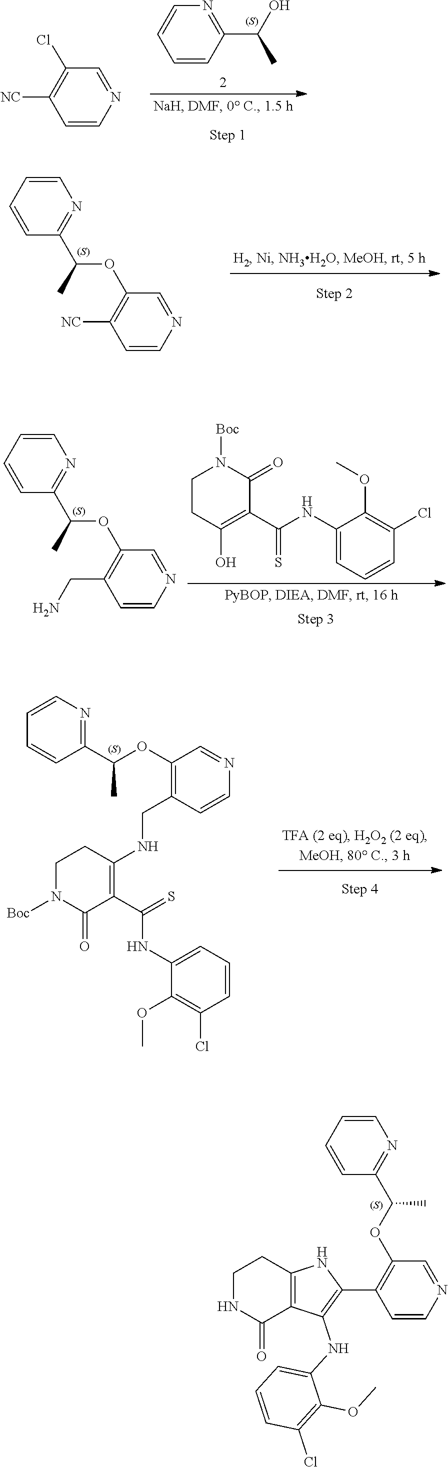

### 224-Step 1

To a stirred suspension of NaH (702 mg, 17.539 mmol) in anhydrous DMF (50 mL) was added (1S)-1-(pyridin-2-yl) ethanol (950 mg, 7.714 mmol) in DMF (5 mL) at −10° C. under N₂. After stirring 0.5 h, 3-chloropyridine-4-carbonitrile (972 mg, 7.012 mmol) in DMF (5 mL) was added to the solution at 0° C. The reaction mixture was stirred at 0° C. for 1 h. After completion, the reaction mixture was quenched with saturated NH₄Cl, extracted with EA. The organic phase was washed with brine, dried over anhydrous Na₂SO₄ and filtered. The filtrate was collected and concentrated. The residue was purified by flash silica gel column chromatography (PE/EA from 0˜70%) to give (S)-3-(1-(pyridin-2-yl) ethoxy) isonicotinonitrile (1 g, 63%) as colorless oil.

MS (ESI): mass calcd. for C₁₃H₁₁N₃O, 225.1, m/z found 226.1 [M+H]⁺.

### 224-Step 2

To a stirred solution of (S)-3-(1-(pyridin-2-yl) ethoxy) isonicotinonitrile (0.97 g, 4.306 mmol) in MeOH (50 mL) and NH₃·H₂O (5 mL) was added Raney-Ni (200 mg) under N₂. The reaction mixture was stirred at room temperature for 5 h under H2. After filtration, the filtrate was collected and concentrated under reduced pressure and vacuum. The residue was purified by flash silica gel column chromatography (DCM/MeOH from 0 10%) to give (S)-(3-(1-(pyridin-2-yl) ethoxy) pyridin-4-yl) methanamine (800 mg, 81%) as an orange red oil.

MS (ESI): mass calcd. for C₁₃H₁₅N₃O, 229.1, m/z found 230.1 [M+H]⁺.

### 224-Step 3

To a stirred solution of (S)-(3-(1-(pyridin-2-yl) ethoxy) pyridin-4-yl) methanamine (500 mg, 2.181 mmol) in DMF (40 mL) were added tert-butyl 5-((3-chloro-2-methoxyphenyl)carbamothioyl)-4-hydroxy-6-oxo-3, 6-dihydropyridine-1(2H)-carboxylate (900 mg, 2.181 mmol), PyBOP (1.702 g, 3.272 mmol), DIEA (846 mg, 6.543 mmol). The reaction mixture was stirred at room temperature for 16 h. After completion, the reaction mixture was quenched with water, extracted with EA. The organic phase was washed with brine, dried over anhydrous Na₂SO₄ and filtered. The filtrate was collected and concentrated. The residue was purified by flash silica gel column chromatography (DCM/MeOH from 0˜10%) to give tert-butyl (S)-5-((3-chloro-2-methoxyphenyl) carbamothioyl)-6-oxo-4-(((3-(1-(pyridin-2-yl)ethoxy)pyridin-4-yl)methyl)amino)-3, 6-dihydropyridine-1(2H)-carboxylate (1.3 g, 80% pure) as off-yellow oil.

MS (ESI): mass calcd. for C₃₁H₃₄ClN₅O₅S, 623.2, m/z found 624.2 [M+H]⁺.

### 224-Step 4

To a stirred solution of tert-butyl (S)-5-((3-chloro-2-methoxyphenyl) carbamothioyl)-6-oxo-4-(((3-(1-(pyridin-2-yl)ethoxy)pyridin-4-yl)methyl)amino)-3, 6-dihydropyridine-1(2H)-carboxylate (500 mg, 0.8 mmol) in MeOH (15 mL) were added H₂O₂ (31%, 182 mg, 1.6 mmol), TFA (183 mg, 1.6 mmol) at 0° C. Then the reaction mixture was stirred at 80° C. for 3 h. After completion, the reaction mixture was cooled to room temperature and concentrated under reduced pressure. The residue was diluted with EA, and the organic phase washed with water, brine, dried over anhydrous Na₂SO₄ and filtered. The filtrate was collected and concentrated. The residue was purified by prep-HPLC with the following conditions: Column: Welch 10 u C18 250×21.2 mm; Mobile Phase A: H₂O (0.1% NH₃), Mobile Phase B: ACN—HPLC; Flow rate: 25 mL/min; Gradient: 45% B to 55% B in 9.5 min; 214 nm; Rt: 8.28 min to give (S)-3-((3-chloro-2-methoxyphenyl)amino)-2-(3-(1-(pyridin-2-yl)ethoxy)pyridin-4-yl)-1, 5, 6, 7-tetrahydro-4H-pyrrolo[3, 2-c]pyridin-4-one (46 mg, 12%) as yellow solid.

MS (ESI): mass calcd. for C₂₆H₂₄ClN₅O₃, 489.2, m/z found 490.2 [M+H]⁺.

¹H NMR (400 MHz, DMSO) δ 12.20 (s, 1H), 8.65 (d, J=4.3 Hz, 1H), 8.38 (s, 1H), 7.96 (d, J=5.0 Hz, 1H), 7.85 (t, J=7.7, 1.5 Hz, 1H), 7.53-7.48 (m, 2H), 7.39 (dd, J=7.0, 5.3 Hz, 1H), 7.27 (d, J=5.0 Hz, 1H), 7.15 (s, 1H), 6.68-6.63 (m, 2H), 6.16 (dd, J=6.8, 2.8 Hz, 1H), 5.90 (q, J=6.3 Hz, 1H), 3.87 (s, 3H), 3.47-3.45 (m, 2H), 3.02-2.93 (m, 2H), 1.66 (d, J=6.3 Hz, 3H).

### Example 225. (R)-3-((3-chloro-2-methoxyphenyl)amino)-2-(3-(1-(pyridin-2-yl)ethoxy)pyridin-4-yl)-1, 5, 6, 7-tetrahydro-4H-pyrrolo[3, 2-c]pyridin-4-one (compound 533)

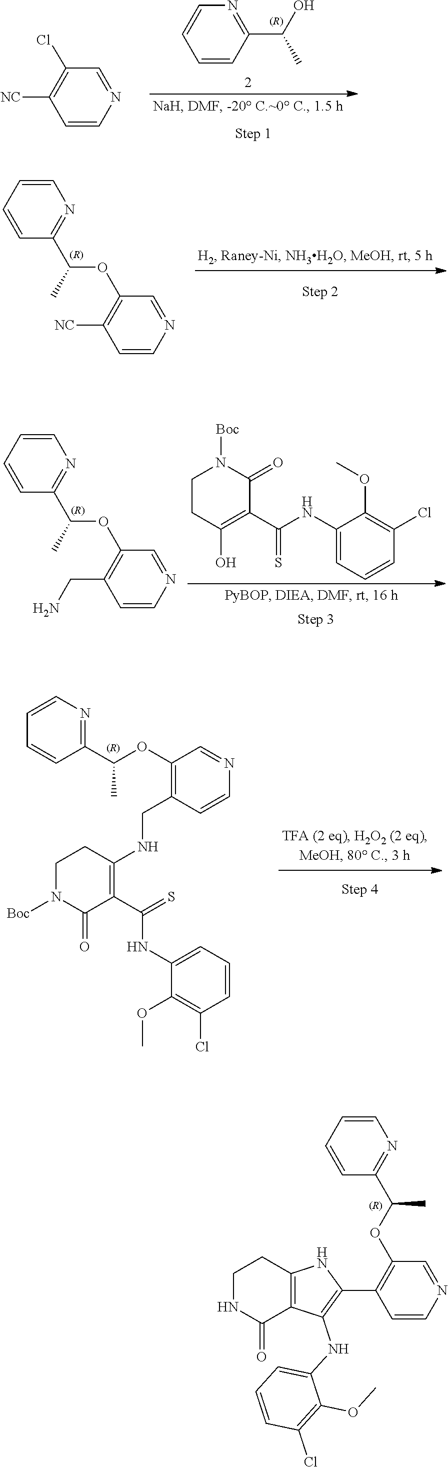

### 225-Step 1

A solution of (1R)-1-(pyridin-2-yl) ethanol (770 mg, 6.252 mmol) in anhydrous DMF (5 mL) was added to the mixture of NaH (568 mg, 14.207 mmol) in anhydrous DMF (20 mL) at 0° C. under N₂. After stirring for 0.5 h, 3-chloropyridine-4-carbonitrile (787 mg, 5.683 mmol) in anhydrous DMF (5 mL) was added to the above mixture. The reaction mixture was stirred at 0° C. for additional 1 h. After completion, the reaction mixture was quenched with ice saturated NH₄Cl aq, extracted with EA. The organic phase was washed with brine, dried over anhydrous Na₂SO₄ and filtered. The filtrate was collected and concentrated. The residue was purified by flash silica gel column chromatography (PE/EA from 0˜65%) to give (R)-3-(1-(pyridin-2-yl) ethoxy) isonicotinonitrile (1 g, 78%) as colorless oil.

MS (ESI): mass calcd. for C₁₃H₁₁N₃O, 225.1, m/z found 226.1 [M+H]⁺.

### 225-Step 2

To a stirred solution of (R)-3-(1-(pyridin-2-yl) ethoxy) isonicotinonitrile (900 mg, 3.996 mmol) in MeOH (50 mL) and NH₃·H₂O (5 mL) was added Raney-Ni (200 mg) under N₂. The reaction mixture was stirred at room temperature for 5 h under H2. After completion, the solvent was collected and concentrated under reduced pressure and vacuum. The residue was purified by flash silica gel column chromatography (DCM/MeOH from 0 10%) to give (R)-(3-(1-(pyridin-2-yl) ethoxy) pyridin-4-yl) methanamine (760 mg, 82%) as a yellow oil.

MS (ESI): mass calcd. for C₁₃H₁₅N₃O, 229.1, m/z found 230.1 [M+H]⁺.

### 225-Step 3

To a stirred solution of (R)-(3-(1-(pyridin-2-yl) ethoxy) pyridin-4-yl) methanamine (500 mg, 2.181 mmol) in DMF (40 mL) were added tert-butyl 5-((3-chloro-2-methoxyphenyl)carbamothioyl)-4-hydroxy-6-oxo-3, 6-dihydropyridine-1(2H)-carboxylate (900 mg, 2.181 mmol), PyBOP (1.702 g, 3.272 mmol), DIEA (846 mg, 6.543 mmol). The reaction mixture was stirred at room temperature for 16 h. After completion, the reaction mixture was quenched with water, extracted with EA. The organic phase was washed with brine, dried over anhydrous Na₂SO₄ and filtered. The filtrate was collected and concentrated. The residue was purified by flash silica gel column chromatography (DCM/MeOH from 0˜10%) to give tert-butyl (R)-5-((3-chloro-2-methoxyphenyl) carbamothioyl)-6-oxo-4-(((3-(1-(pyridin-2-yl)ethoxy)pyridin-4-yl)methyl)amino)-3, 6-dihydropyridine-1(2H)-carboxylate (1.3 g, 80% pure) as dark-yellow oil.

MS (ESI): mass calcd. for C₃₁H₃₄ClN₅O₅S, 623.2, m/z found 624.2 [M+H]⁺.

### 225-Step 4

To a stirred solution of tert-butyl (R)-5-((3-chloro-2-methoxyphenyl) carbamothioyl)-6-oxo-4-(((3-(1-(pyridin-2-yl)ethoxy)pyridin-4-yl)methyl)amino)-3, 6-dihydropyridine-1(2H)-carboxylate (500 mg, 0.8 mmol) in MeOH (15 mL) were added H₂O₂ (31%, 182 mg, 1.6 mmol), TFA (183 mg, 1.6 mmol) at 0° C. Then the reaction mixture was stirred at 80° C. for 3 h. After completion, the reaction mixture was cooled to room temperature and concentrated under reduced pressure. The residue was diluted with EA, and the organic phase washed with water, brine, dried over anhydrous Na₂SO₄ and filtered. The filtrate was collected and concentrated. The residue was purified by prep-HPLC with the following conditions: Column: Xbridge 5u C18 150×30 mm; Mobile Phase A: H₂O (0.1% FA), Mobile Phase B: ACN—HPLC; Flow rate: 30 mL/min; Gradient: 32% B to 45% B in 9 min; 214 nm; Rt: 8 min to give (R)-3-((3-chloro-2-methoxyphenyl)amino)-2-(3-(1-(pyridin-2-yl)ethoxy)pyridin-4-yl)-1, 5, 6, 7-tetrahydro-4H-pyrrolo[3, 2-c]pyridin-4-one (121 mg, 30%) as yellow solid.

MS (ESI): mass calcd. for C₂₆H₂₄ClN₅O₃, 489.2, m/z found 490.2 [M+H]⁺.

¹H NMR (400 MHz, DMSO) δ 12.29 (s, 1H), 8.66 (d, J=4.4 Hz, 1H), 8.43 (s, 1H), 8.02 (d, J=5.2 Hz, 1H), 7.87 (t, J=7.7, 1.6 Hz, 1H), 7.63 (s, 1H), 7.51 (d, J=7.9 Hz, 1H), 7.40 (dd, J=6.9, 5.1 Hz, 1H), 7.33 (d, J=5.2 Hz, 1H), 7.18 (s, 1H), 6.72-6.65 (m, 2H), 6.17 (dd, J=7.5, 2.0 Hz, 1H), 5.93 (q, J=6.3 Hz, 1H), 3.88 (s, 3H), 3.48 (t, J=6.1 Hz, 2H), 3.00 (dd, J=6.8, 4.8 Hz, 2H), 1.68 (d, J=6.4 Hz, 3H).

### Example 226. (R)-3-((3-chloro-2-methoxyphenyl)amino)-2-(3-(2-(pyridin-2-yl)propoxy)pyridin-4-yl)-1,5,6,7-tetrahydro-4H-pyrrolo[3,2-c]pyridin-4-one/(S)-3-((3-chloro-2-methoxyphenyl)amino)-2-(3-(2-(pyridin-2-yl)propoxy)pyridin-4-yl)-1,5,6,7-tetrahydro-4H-pyrrolo[3,2-c]pyridin-4-one (compound 534/535)

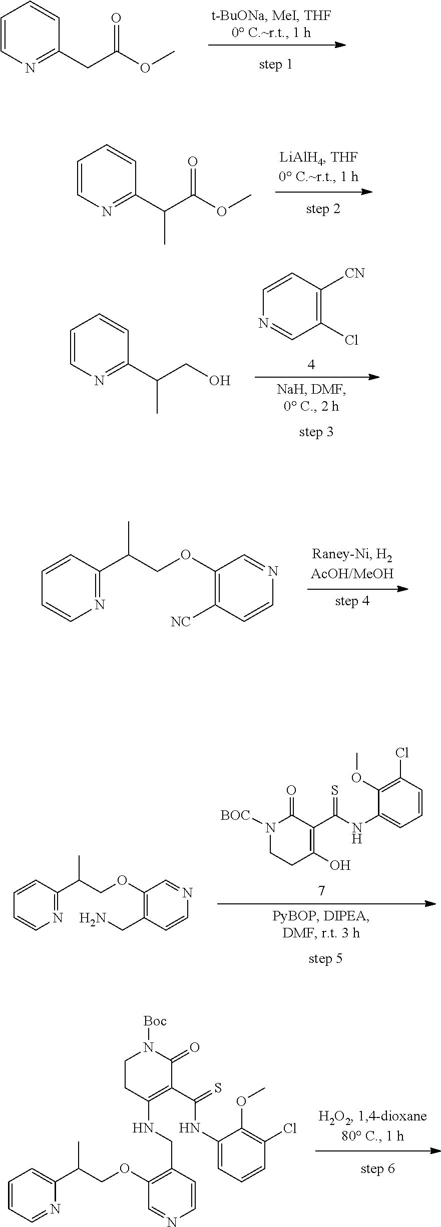

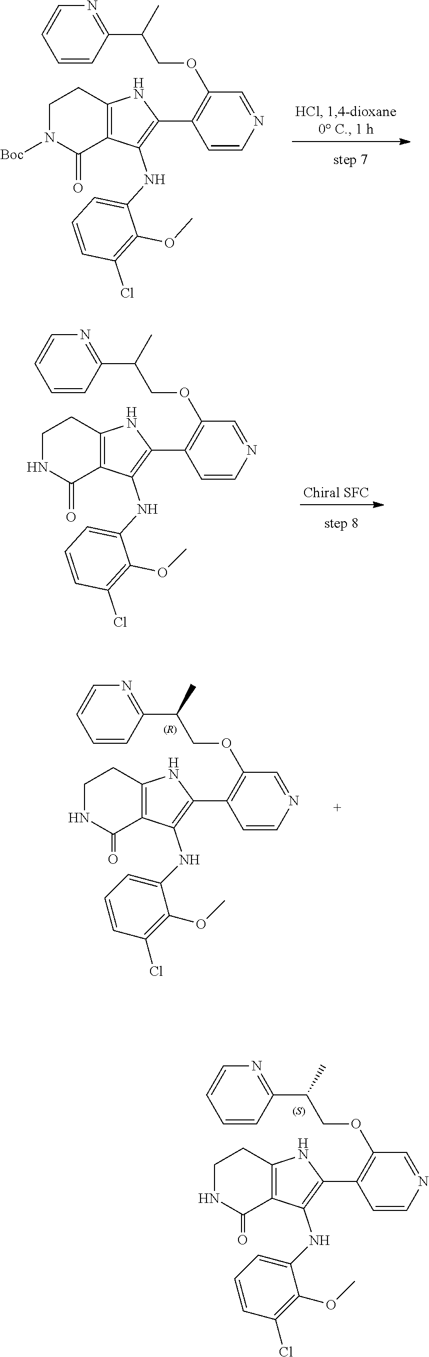

### 226-Step 1

To a solution of methyl 2-(pyridin-2-yl)acetate (5 g, 33.11 mmol) and t-BuONa (3.34 g, 34.77 mmol) in THF (175 mL) was stirred under nitrogen at 0° C. for 30 min. Then a solution of Mel (9.4 g, 66.23 mmol) in THF (20 mL) was added thereto dropwise at 0° C. and the reaction mixture was stirred at room temperature for 1 h. After completion, the mixture was quenched with saturated aqueous NH₄Cl (100 mL) and extracted with EtOAc (200 mL×3). The combined organic layers were washed with brine (100 mL), dried over Na₂SO₄ and concentrated in vacuo to give the crude, which was purified by silica gel column chromatography (eluent: PE/EtOAc=2/1) to give the methyl 2-(pyridin-2-yl)propanoate (4.5 g, yield: 82%) as a yellow oil.

MS (ESI): mass calcd. for C₉H₁₁NO₂, 165.08, m/z found 166.2 [M+H]⁺.

### 226-Step 2

To a solution of methyl 2-(pyridin-2-yl)propanoate (4.5 g, 27.3 mmol) in THF (150 mL) was added LiAlH₄ (1M solution in THF, 35.4 mL, 35.4 mmol) dropwise at 0° C. under nitrogen. The mixture was stirred at room temperature for 1 h. After completion, the reaction mixture was quenched with Na₂SO₄·10H₂O, filtered through celite and the filter cake was washed with MeOH (20 mL). The filtrate was concentrated in vacuo to give the crude, which was purified by silica gel column chromatography (eluent: PE/EtOAc=2/1) to give 2-(pyridin-2-yl)propan-1-ol (2.8 g, yield: 75%) as a yellow oil.

MS (ESI): mass calcd. for C₈H₁₁NO, 137.08, m/z found 138.2 [M+H]⁺.

### 226-Step 3

To a solution of 2-(pyridin-2-yl)propan-1-ol (1.5 g, 10.95 mmol) in DMF (75 mL) was added NaH (60% dispersion in mineral oil, 438 mg, 10.95 mmol) at 0° C. and stirred for 1 h under N₂. Then a solution of 3-chloroisonicotinonitrile (1.51 g, 10.95 mmol) in DMF (15 mL) was added thereto and stirred at 0° C. for 1 h under N₂. After completion, the reaction was quenched with saturated aqueous NH₄Cl (100 mL) and extracted with EtOAc (100 mL×3). The combined organic layers were washed with brine (80 mL), dried over Na₂SO₄ and concentrated in vacuo to give the crude, which was purified by silica gel column chromatography (eluent: PE/EtOAc=2/1) to give the 3-(2-(pyridin-2-yl)propoxy)isonicotinonitrile (1.6 g, yield: 61%) as a yellow oil.

MS (ESI): mass calcd. for C₁₄H₁₃N₃O, 239.11, m/z found 240.1 [M+H]⁺.

### 226-Step 4

A mixture of 3-(2-(pyridin-2-yl)propoxy)isonicotinonitrile (1.6 g, 6.69 mmol) and Raney-Ni (1.0 g) in MeOH (60 mL) and AcOH (15 mL) was stirred at room temperature for 2 h under hydrogen atmosphere. After completion, the mixture was filtered through celite and the filter cake was washed with MeOH (50 mL). The filtrate was concentrated in vacuo to give the crude, which was purified by silica gel column chromatography (eluent: DCM/MeOH/NH₄OH=10/1/0.1) to afford (3-(2-(pyridin-2-yl)propoxy)pyridin-4-yl)methanamine (1.1 g, yield: 68%) as a yellow oil.

MS (ESI): mass calcd. for C₁₄H₁₇N₃O, 243.14, m/z found 244.2 [M+H]⁺.

### 226-Step 5

A solution of tert-butyl 5-((3-chloro-2-methoxyphenyl)carbamothioyl)-4-hydroxy-6-oxo-3,6-dihydropyridine-1(2H)-carboxylate (678 mg, 1.65 mmol), (3-(2-(pyridin-2-yl)propoxy)pyridin-4-yl)methanamine (400 mg, 1.65 mmol), PyBOP (1.11 g, 2.14 mmol) and DIPEA (1.07 g, 8.23 mmol) in DMF (25 mL) was stirred at room temperature for 3 h. After completion, the resulting mixture was diluted with water (50 mL) and extracted with EtOAc (50 mL×3). The combined organic layers were washed with brine (50 mL), dried over anhydrous Na₂SO₄, filtered and concentrated in vacuo to give the crude, which was purified by silica gel column chromatography (DCM/MeOH=20/1) to afford tert-butyl 5-((3-chloro-2-methoxyphenyl)carbamothioyl)-6-oxo-4-(((3-(2-(pyridin-2-yl)propoxy)pyridin-4-yl)methyl)amino)-3,6-dihydropyridine-1(2H)-carboxylate (660 mg, yield: 63%) as a yellow solid.

MS (ESI): mass calcd. for C₃₂H₃₆ClN₅O₅S, 637.21, m/z found 638.2 [M+H]⁺.

### 226-Step 6

To a solution of tert-butyl 5-((3-chloro-2-methoxyphenyl)carbamothioyl)-6-oxo-4-(((3-(2-(pyridin-2-yl)propoxy)pyridin-4-yl)methyl)amino)-3, 6-dihydropyridine-1(2H)-carboxylate (350 mg 0.55 mmol) in 1,4-dioxane (20 mL) was added H₂O₂ (30% solution in H₂O, 0.5 mL, 1.65 mL). The reaction was stirred at 80° C. for 1 h. After completion, the reaction was concentrated under reduced pressure to give the crude, which was purified with Prep-TLC (eluent: DCM/MeOH=15/1) to give the tert-butyl 3-((3-chloro-2-methoxyphenyl)amino)-4-oxo-2-(3-(2-(pyridin-2-yl)propoxy)pyridin-4-yl)-1,4,6,7-tetrahydro-5H-pyrrolo[3,2-c]pyridine-5-carboxylate (155 mg, yield: 47%) as a yellow solid.

MS (ESI): mass calcd. for C₃₂H₃₄ClN₅O₅, 603.22, m/z found 604.2 [M+H]⁺.

### 226-Step 7

To a solution of tert-butyl 3-((3-chloro-2-methoxyphenyl)amino)-4-oxo-2-(3-(2-(pyridin-2-yl)propoxy)pyridin-4-yl)-1,4,6,7-tetrahydro-5H-pyrrolo[3,2-c]pyridine-5-carboxylate (155 mg, 0.26 mmol) in 1,4-dioxane (10 mL) was added HCl (4M solution in 1,4-dioxane, 2 mL). The resulting mixture was stirred at 0° C. for 1 h. The reaction mixture was concentrated to give the crude product, which was purified by Prep-TLC (eluent: DCM/MeOH=15/1) to give 3-((3-chloro-2-methoxyphenyl)amino)-2-(3-(2-(pyridin-2-yl)propoxy)pyridin-4-yl)-1,5,6,7-tetrahydro-4H-pyrrolo[3,2-c]pyridin-4-one (110 mg, yield: 84%) as a yellow solid.

MS (ESI): mass calcd. for C₂₇H₂₆ClN₅O₃, 503.17, m/z found 504.2 [M+H]⁺.

### 226-Step 8

3-((3-chloro-2-methoxyphenyl)amino)-2-(3-(2-(pyridin-2-yl)propoxy)pyridin-4-yl)-1,5,6,7-tetrahydro-4H-pyrrolo[3,2-c]pyridin-4-one (110 mg, 0.22 mmol) was separated by SFC (Daicel CHIRALPAK OD-H, 20×250 mm, 5 μm 70/30 CO2/MeOH [0.2% NH₃(7M Solution in MeOH)], 50 g/min, 120 bar, 35° C.) to give two enantiomers: (R)-3-((3-chloro-2-methoxyphenyl)amino)-2-(3-(2-(pyridin-2-yl)propoxy)pyridin-4-yl)-1,5,6,7-tetrahydro-4H-pyrrolo[3,2-c]pyridin-4-one (33 mg, yield: 30%) as a white solid and (S)-3-((3-chloro-2-methoxyphenyl)amino)-2-(3-(2-(pyridin-2-yl)propoxy)pyridin-4-yl)-1,5,6,7-tetrahydro-4H-pyrrolo[3,2-c]pyridin-4-one (36 mg, yield: 33%) as a white solid.

P1: MS (ESI): mass calcd. for C₂₇H₂₆ClN₅O₃, 503.17, m/z found 504.2 [M+H]⁺.

P1: ¹H NMR (400 MHz, DMSO) δ 11.66 (s, 1H), 8.56 (d, J=3.9 Hz, 1H), 8.33 (s, 1H), 7.97 (d, J=5.0 Hz, 1H), 7.82-7.72 (m, 1H), 7.48-7.39 (m, 2H), 7.27 (m, 2H), 7.13 (s, 1H), 6.71-6.62 (m, 2H), 6.04 (m, 1H), 4.44-4.35 (m, 2H), 3.88 (s, 3H), 3.53 (m, 1H), 3.43 (m, 2H), 3.00-2.83 (m, 2H), 1.40 (d, J=7.1 Hz, 3H).

P2: MS (ESI): mass calcd. for C₂₇H₂₆ClN₅O₃, 503.17, m/z found 504.2 [M+H]⁺.

P2: ¹H H NMR (400 MHz, DMSO) δ 11.66 (s, 1H), 8.56 (d, J=3.9 Hz, 1H), 8.33 (s, 1H), 7.97 (d, J=5.0 Hz, 1H), 7.76 (m, 1H), 7.48-7.37 (m, 2H), 7.28 (m, 2H), 7.13 (s, 1H), 6.71-6.62 (m, 2H), 6.04 (m, 1H), 4.44-4.34 (m, 2H), 3.88 (s, 3H), 3.53 (m, 1H), 3.44 (m, 2H), 2.99-2.84 (m, 2H), 1.40 (d, J=7.1 Hz, 3H).

### Example 227. 3-((3-chloro-2-methoxyphenyl)amino)-2-(3-(2-methyl-2-(pyridin-2-yl)propoxy)pyridin-4-yl)-1,5,6,7-tetrahydro-4H-pyrrolo[3,2-c]pyridin-4-one (compound 536)

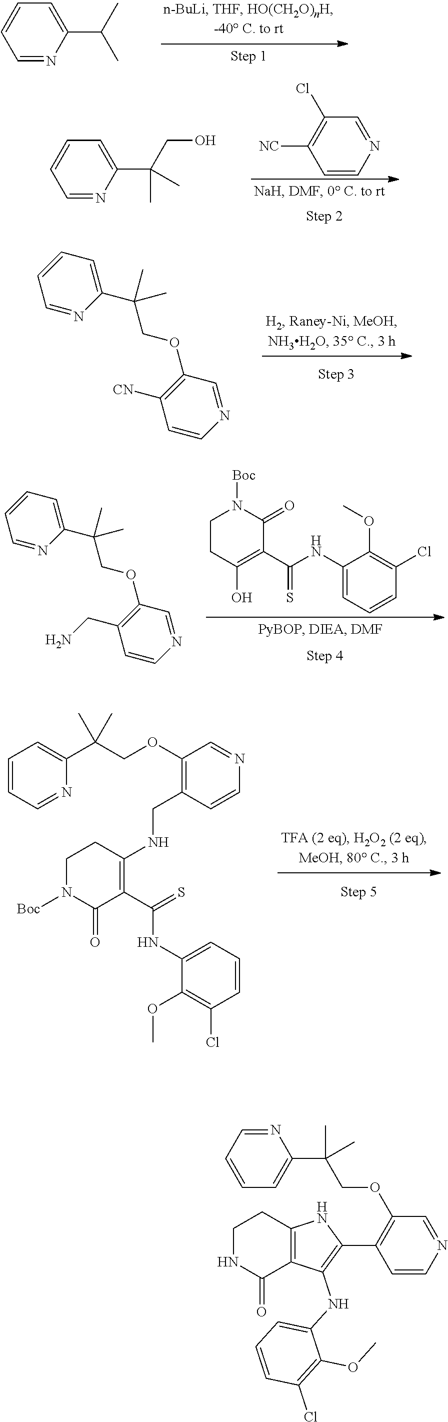

### 227-Step 1

To a solution of 2-isopropylpyridine (9.5 g, 0.0784 mol) in THF (100 mL) was added n-BuLi (43.1 mL, 0.0862 mol, 2 M in hexane) at 0° C. The reaction mixture was stirred at room temperature for 0.5 h, paraformaldehyde (4.7 g, 0.1568 mol) was added at −40° C. The reaction mixture was stirred at −40° C. for 0.5 h and room temperature for 1.5 h. Saturated aqueous 3N NaOH was added. The mixture was extracted with EA (200 mL×3). The organic layer was dried over anhydrous Na₂SO₄, filtered and concentrated under reduced pressure to give the crude product. The residue was purified by Flash Chromatography to give 2-methyl-2-(pyridin-2-yl)propan-1-ol (3 g, 24%) as a yellow solid.

MS (ESI): mass calcd. for C₉H₁₃NO, 151.10, m/z found 152.1 [M+H]⁺.

### 227-Step 2

To a solution of 2-methyl-2-(pyridin-2-yl)propan-1-ol (3 g, 0.0198 mol) in DMF (40 mL) was added sodium hydrogen (0.95 g, 0.0396 mol) at 0° C. The reaction was stirred at 0° C. for 0.5 h, 3-chloropyridine-4-carbonitrile (2.74 g, 0.0198 mol) was added at 0° C. The reaction was stirred at room temperature for 1 h. The mixture was diluted with water and extracted with EA (100 mL×3). The organic layer was dried over anhydrous Na₂SO₄, filtered and concentrated under reduced pressure to give the crude product. The residue was purified by Flash Chromatography to give a residue and purified by Flash Chromatography to give 3-(2-methyl-2-(pyridin-2-yl)propoxy)isonicotinonitrile (4 g, 67%) as a yellow solid.

MS (ESI): mass calcd. for C₁₅H₁₅N₃O, 253.12, m/z found 254.1 [M+H]⁺.

### 227-Step 3

To a solution of 3-[2-methyl-2-(pyridin-2-yl)propoxy]pyridine-4-carbonitrile (1 g, 0.0039 mol) in MeOH (20 mL) was added raney ni (0.23 g, 0.0039 mol) under H2. The reaction mixture was stirred at 35° C. under H2 for 2.5 h. The solution was filtered, filtrate was collected and concentrated in vacuo to give the crude product. The residue was purified by Flash Chromatography to give a residue and purified by Flash Chromatography to give (3-(2-methyl-2-(pyridin-2-yl)propoxy)pyridin-4-yl)methanamine (0.9 g, 85%) as a yellow solid.

MS (ESI): mass calcd. for C₁₅H₁₉N₃O, 257.15, m/z found 258.1 [M+H]⁺.

### 227-Step 4

To a solution of {3-[2-methyl-2-(pyridin-2-yl)propoxy]pyridin-4-yl}methanamine (900 mg, 3.4974 mmol), tert-butyl {3-[(3-chloro-2-methoxyphenyl)carbamothioyl]-4-hydroxy-2-oxo-5,6-dihydropyridin-1-yl} formate (1516.27 mg, 3.6722 mmol) in DMF (20 mL) were added N,N-Diisopropylethylamine (1356.01 mg, 10.4922 mmol) and PYBOP (2730.02 mg, 5.2461 mmol). The reaction was stirred at room temperature for 4 h. The mixture was diluted with water and extracted with EA (50 mL×3). The organic layer was dried over anhydrous Na₂SO₄, filtered and concentrated under reduced pressure to give the crude product. The residue was purified by Flash Chromatography to give tert-butyl 5-((3-chloro-2-methoxyphenyl)carbamothioyl)-4-(((3-(2-methyl-2-(pyridin-2-yl)propoxy)pyridin-4-yl)methyl)amino)-6-oxo-3, 6-dihydropyridine-1(2H)-carboxylate (700 mg, 29%) as a yellow solid.

MS (ESI): mass calcd. for C₃₃H₃₈ClN₅O₅S, 651.23, m/z found 652.0/654.0 [M+H]⁺.

### 227-Step 5

To a solution of tert-butyl {3-[(3-chloro-2-methoxyphenyl)carbamothioyl]-4-[({3-[2-methyl-2-(pyridin-2-yl)propoxy]pyridin-4-yl}methyl)amino]-2-oxo-5,6-dihydropyridin-1-yl} formate (300 mg, 0.46 mmol) in MeOH (10 mL) were added trifluoroacetic acid (104.9 mg, 0.92 mol) and Hydrogen peroxide (31.29 mg, 0.92 mol) at 0° C. The reaction was stirred at 80° C. for 3 h. Saturated aqueous Na₂SO₃ was added. The mixture was extracted with EA (30 mL×3). The organic layer was dried over anhydrous Na₂SO₄, filtered and concentrated under reduced pressure to give the crude product. The residue was purified by prep-HPLC to give 3-((3-chloro-2-methoxyphenyl)amino)-2-(3-(2-methyl-2-(pyridin-2-yl)propoxy)pyridin-4-yl)-1,5,6,7-tetrahydro-4H-pyrrolo[3,2-c]pyridin-4-one (54.7 mg, 23%) as a yellow solid.

MS (ESI): mass calcd. for C₂₈H₂₈ClN₅O₃, 517.19, m/z found 518.0 [M+H]⁺.

¹H NMR (400 MHz, DMSO) δ 11.85 (s, 1H), 8.58 (dd, J=4.0, 8.0 Hz, 1H), 8.37 (s, 1H), 8.04 (d, J=8.0 Hz, 1H), 7.84-7.80 (m, 1H), 7.57-7.55 (m, 2H), 7.34 (d, J=8.0 Hz, 1H), 7.31-7.28 (m, 1H), 7.22 (s, 1H), 6.72-6.66 (m, 2H), 6.05 (dd, J=4.0, 8.0 Hz, 1H), 4.32 (s, 2H), 3.88 (s, 3H), 3.47-3.43 (m, 2H), 2.91 (t, J=8.0 Hz, 2H), 1.52 (s, 6H).

### Example 228. 3-((3-chloro-2-methoxyphenyl)amino)-2-(3-((6-methoxypyridin-2-yl)methoxy)pyridin-4-yl)-1, 5, 6, 7-tetrahydro-4H-pyrrolo[3, 2-c]pyridin-4-one (compound 537)

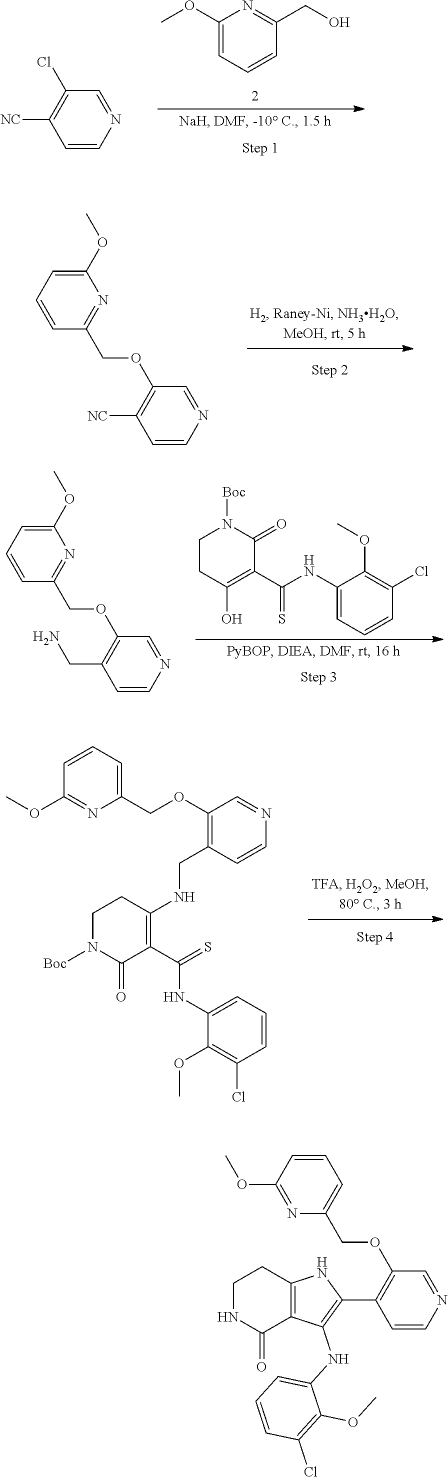

### 228-Step 1

To a stirred suspension of NaH (60%, 377 mg, 9.42 mmol) in DMF (50 mL) was added (6-methoxypyridin-2-yl) methanol (1.42 g, 10.2 mmol) in DMF (5 mL) at −10° C. under N₂. After stirring 0.5 h, 3-chloropyridine-4-carbonitrile (1.1 g, 7.85 mmol) in DMF (5 mL) was added to the solution at −10° C. Then the reaction mixture was stirred at −10° C. for 1 h. After completion, the reaction mixture was quenched with ice saturated NH₄C1 aq, extracted with EA. The organic phase was washed with brine, dried over anhydrous Na₂SO₄ and filtered. The filtrate was collected and concentrated. The residue was purified by flash silica gel column chromatography (EA/PE from 0˜40%) to give 3-((6-methoxypyridin-2-yl) methoxy) isonicotinonitrile (1.3 g, 68%) as white solid.

MS (ESI): mass calcd. for C₁₃H₁₁N₃O₂, 241.1, m/z found 242.1 [M+H]⁺.

### 228-Step 2

To a stirred solution of 3-[(6-methoxypyridin-2-yl) methoxy] pyridine-4-carbonitrile (250 mg, 1.036 mmol) in MeOH (25 mL) and NH₃·H₂O (2.5 mL) was added Raney-Ni (100 mg) under N₂. The reaction mixture was stirred at room temperature for 5 h under H2. After completion, the solvent was collected and concentrated under reduced pressure and vacuum. The residue was purified by flash silica gel column chromatography (MeOH/DCM from 0˜10%) to give (3-((6-methoxypyridin-2-yl) methoxy) pyridin-4-yl) methanamine (220 mg, 86%) as a white solid.

MS (ESI): mass calcd. for C₁₃H₁₅N₃O₂, 245.1, m/z found 246.1 [M+H]⁺.

### 228-Step 3

To a stirred solution of (3-((6-methoxypyridin-2-yl) methoxy) pyridin-4-yl) methanamine (220 mg, 0.897 mmol) in DMF (10 mL) were added tert-butyl 5-((3-chloro-2-methoxyphenyl) carbamothioyl)-4-hydroxy-6-oxo-3, 6-dihydropyridine-1(2H)-carboxylate (371 mg, 0.897 mmol), PyBOP (701 mg, 1.346 mmol), DIEA (348 mg, 2.691 mmol). The reaction mixture was stirred at room temperature for 16 h. After completion, the reaction mixture was quenched with water, extracted with EA. The organic phase was washed with brine, dried over anhydrous Na₂SO₄ and filtered. The filtrate was collected and concentrated. The residue was purified by flash silica gel column chromatography (MeOH/DCM from 0˜10%) to give tert-butyl 5-((3-chloro-2-methoxy phenyl)carbamothioyl)-4-(((3-((6-methoxypyridin-2-yl)methoxy)pyridin-4-yl)methyl)amino)-6-oxo-3,6-dihydropyridine-1(2H)-carboxylate (270 mg, 80% pure) as yellow oil.

MS (ESI): mass calcd. for C₃₁H₃₄ClN₅O₆S, 639.2, m/z found 640.2 [M+H]⁺.

### 228-Step 4

To a stirred solution of tert-butyl 5-((3-chloro-2-methoxy phenyl)carbamothioyl)-4-(((3-((6-methoxypyridin-2-yl)methoxy)pyridin-4-yl)methyl)amino)-6-oxo-3, 6-dihydropyridine-1(2H)-carboxylate (220 mg, 0.344 mmol) in MeOH (10 mL) were added H₂O₂ (31%, 78 mg, 0.688 mmol), TFA (79 mg, 0.688 mmol) at 0° C. Then the reaction mixture was stirred at 80° C. for 3 h. After completion, the reaction mixture was cooled to room temperature and concentrated under reduced pressure. The residue was diluted with EA, and the organic phase washed with water, brine, dried over anhydrous Na₂SO₄ and filtered. The filtrate was collected and concentrated. The residue was purified by prep-HPLC with the following conditions: Column: Welch 10 u C18 250×21.2 mm; Mobile Phase A: H₂O (0.1% FA), Mobile Phase B: ACN—HPLC; Flow rate: 30 mL/min; Gradient: 32% B to 45% B in 9 min; 214 nm; Rt: 8 min to give 3-((3-chloro-2-methoxyphenyl)amino)-2-(3-((6-methoxypyridin-2-yl)methoxy)pyridin-4-yl)-1, 5, 6, 7-tetrahydro-4H-pyrrolo[3, 2-c]pyridin-4-one (72 mg, 41%) as yellow solid.

MS (ESI): mass calcd. for C₂₆H₂₄ClN₅O₄, 505.2, m/z found 506.2 [M+H]⁺.

¹H NMR (400 MHz, DMSO) δ 11.40 (s, 1H), 8.38 (s, 1H), 8.03 (d, J=5.0 Hz, 1H), 7.73 (t, J=7.8 Hz, 1H), 7.48 (s, 1H), 7.31 (d, J=5.0 Hz, 1H), 7.10 (s, 1H), 7.01 (d, J=7.3 Hz, 1H), 6.78 (d, J=8.3 Hz, 1H), 6.66-6.60 (m, 2H), 6.14 (dd, J=7.2, 2.3 Hz, 1H), 5.35 (s, 2H), 3.82 (d, J=6.4 Hz, 6H), 3.44-3.41 (m, 2H), 2.87 (t, J=6.8 Hz, 2H).

### Example 229. 2-[3-(benzyloxy)pyridin-4-yl]-3-[(3-fluoro-2-methylphenyl)amino]-1H,5H,6H,7H-pyrrolo[3,2-c]pyridin-4-one (compound 538)

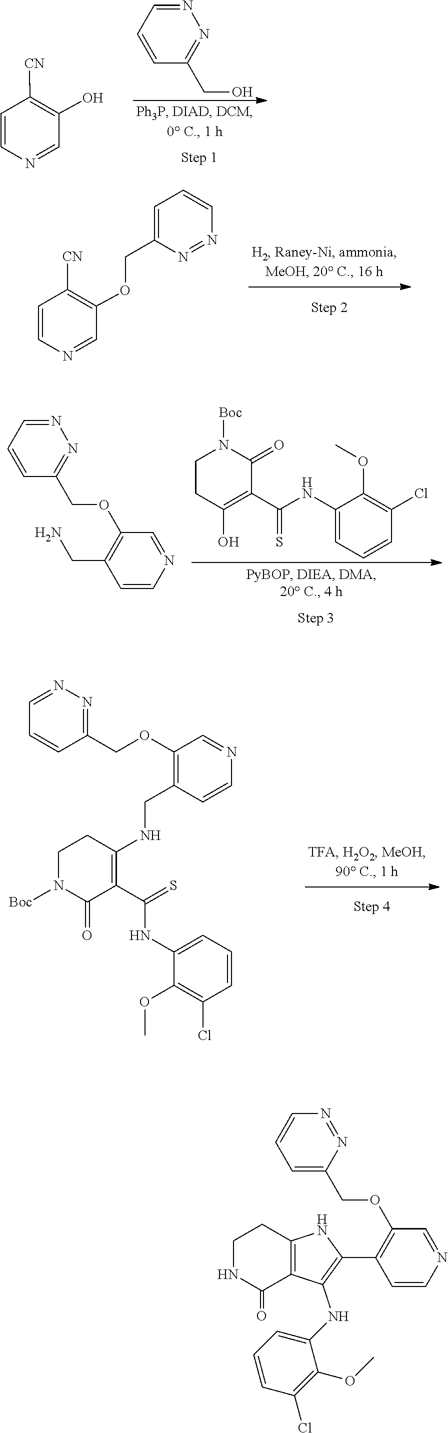

### 229-Step 1

To a solution of 3-hydroxypyridine-4-carbonitrile (450 mg, 3.75 mmol), pyridazin-3-ylmethanol (495 mg, 4.49 mmol) and PPh₃ (1.20 g, 4.49 mmol) in DCM (20 mL) was added DIAD (909 mg, 4.49 mmol) dropwise at 0° C. The reaction mixture was stirred at 0° C. for 1 h. The mixture was concentrated to give the crude product. The crude was purified by flash chromatography (PE/EA from 0˜40%) to give the product 3-(pyridazin-3-ylmethoxy)pyridine-4-carbonitrile (260 mg, 33% yield) as yellow oil.

MS (ESI): mass calcd. for C₁₁H₈N₄O, 212.1, m/z found 213.1 [M+H]⁺.

### 229-Step 2

To a solution of 3-(pyridazin-3-ylmethoxy)pyridine-4-carbonitrile (260 mg, 1.22 mmol) in NH₃-MeOH (7 M in methanol, 5 mL) and MeOH (20 mL) was added Raney-Ni (260 mg) under N₂. The reaction was stirred at 20° C. for 16 h under H2. After completion, the solvent was collected and concentrated under reduced pressure. The residue was purified by flash silica gel column chromatography (DCM/MeOH from 0˜10%) to give [3-(pyridazin-3-ylmethoxy)pyridin-4-yl]methanamine (250 mg, 94% yield) as yellow oil.

MS (ESI): mass calcd. for C₁₁H₁₂N₄O, 216.1, m/z found 217.1 [M+H]⁺.

### 229-Step 3

To a solution of [3-(pyridazin-3-ylmethoxy)pyridin-4-yl]methanamine (250 mg, 1.16 mmol), tert-butyl {3-[(3-chloro-2-methoxyphenyl)carbamothioyl]-4-hydroxy-2-oxo-5,6-dihydropyridin-1-yl} formate (526 mg, 1.27 mmol) and PYBOP (1.2 g, 2.3 mmol) in DMA (10 mL) was added DIPEA (750 mg, 5.8 mmol). The reaction mixture was stirred at 20° C. for 4 h. After completion, the reaction mixture was quenched with water, extracted with EA. The organic phase was washed with brine, dried over anhydrous Na₂SO₄ and filtered. The filtrate was collected and concentrated. The residue was purified by flash silica gel column chromatography (DCM/MeOH from 0˜10%) to give tert-butyl {3-[(3-chloro-2-methoxyphenyl)carbamothioyl]-4-{[(3-{[6-(difluoromethyl)pyridin-2-yl]methoxy}pyridin-4-yl)methyl]amino}-2-oxo-5,6-dihydropyridin-1-yl} formate (280 mg, 48%) as a yellow solid.

### 229-Step 4

To a solution of tert-butyl {3-[(3-chloro-2-methoxyphenyl)carbamothioyl]-2-oxo-4-({[3-(pyridazin-3-ylmethoxy)pyridin-4-yl]methyl}amino)-5,6-dihydropyridin-1-yl} formate (160 mg, 0.26 mmol) in 1,4-dioxane (5.0 mL) was added TFA (60 mg, 0.53 mmol) and H₂O₂ (30%, 89 mg, 0.78 mmol). The reaction was stirred for 1 h at 90° C. After completion, the reaction mixture was cooled to room temperature and concentrated under reduced pressure. The residue was diluted with EA, and the organic phase washed with water, brine, dried over anhydrous Na₂SO₄ and filtered. The filtrate was collected and concentrated. The residue was purified by prep-HPLC with the following conditions: Column: Xbridge 5u C18 150×19 mm; Mobile Phase A: H₂O (0.1% FA), Mobile Phase B: ACN—HPLC; Flow rate: 20 mL/min; Gradient: 15% B to 45% B in 8 min; 214 nm; Rt: 5.7 min to give 2-[3-(benzyloxy)pyridin-4-yl]-3-[(3-fluoro-2-methylphenyl)amino]-1H,5H,6H,7H-pyrrolo[3,2-c]pyridin-4-one (43 mg, 29%) as a yellow solid.

MS (ESI): mass calcd. for C₂₄H₂₁ClN₆O₃, 476.2, m/z found 477.2 [M+H]⁺.

¹H NMR (400 MHz, DMSO) δ 12.27 (s, 1H), 9.28 (dd, J=4.0, 2.4 Hz, 1H), 8.47 (s, 1H), 8.05 (d, J=5.2 Hz, 1H), 7.85-7.79 (m, 1H), 7.53 (s, 1H), 7.35 (d, J=4.8 Hz, 1H), 7.15 (s, 1H), 6.69 (s, 1H), 6.68 (d, J=1.6 Hz, 1H), 6.17 (dd, J=5.6, 4.4 Hz, 1H), 5.73 (s, 2H), 3.87 (s, 3H), 3.46-3.44 (m, 2H), 2.98 (t, J=6.8 Hz, 2H).

### Example 230. 3-((3-chloro-2-methoxyphenyl)amino)-2-(3-((6-(difluoromethyl)pyridin-2-yl)methoxy)pyridin-4-yl)-6,7-dihydro-1H-pyrrolo[3,2-c]pyridin-4(5H)-one (compound 539)

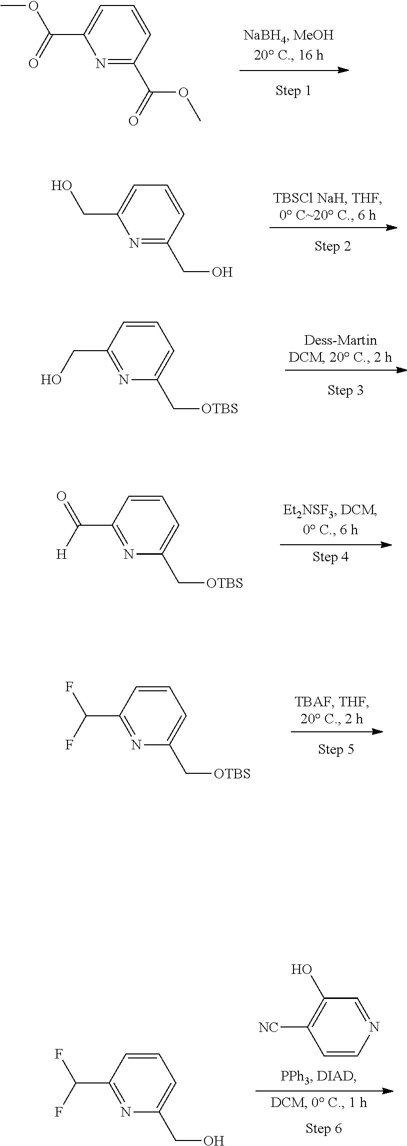

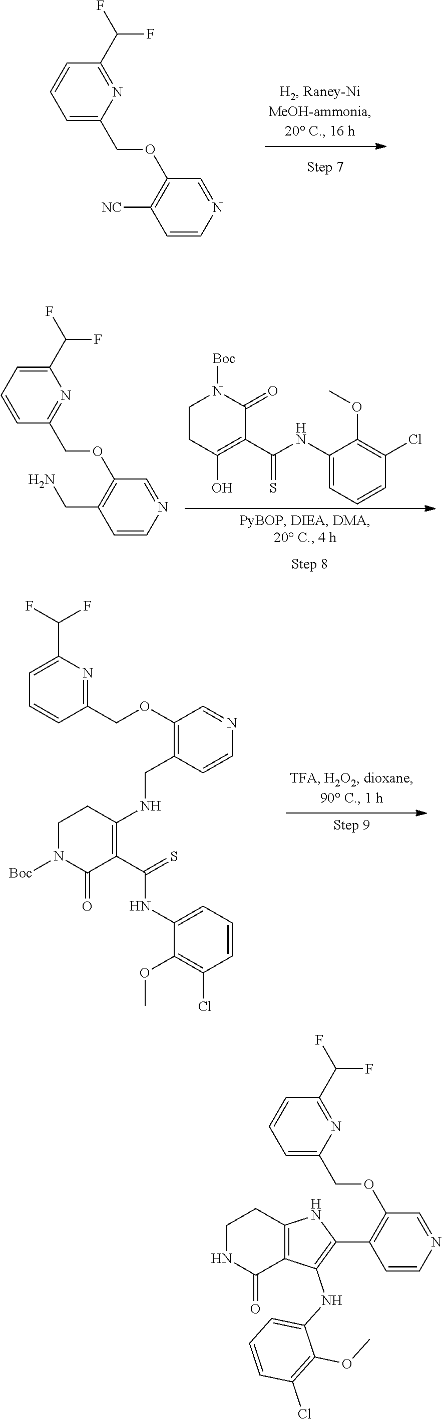

### 230-Step 1

To a solution of 2,6-dimethyl pyridine-2,6-dicarboxylate (5.0 g, 0.022 mol) in MeOH (50 mL) was added sodium borohydride (3.3 g, 0.088 mol) in portions at 0° C. The reaction was stirred at 20° C. for 16 h. After completion, the reaction mixture was quenched with saturated Na₂CO₃ (aq. 10 mL) and the solvent was concentrated to residue. Then DCM/MeOH (10/1, 200 mL) was added and filtrated. The filtrate was dried over Na₂SO₄ and filtrated. The filtrate was concentrated to give pyridine-2,6-diyldimethanol (3.0 g, 96%) as a white solid.

MS (ESI): mass calcd. for C₇H₉NO₂, 139.1, m/z found 140.2 [M+H]⁺.

### 230-Step 2

To a stirred suspension of NaH (1.0 g, 60% in mineral oil, 0.025 mol)) in THF (25 mL) was added pyridine-2,6-diyldimethanol (3.0 g, 0.023 mol) at 0° C. under N₂. After stirring 0.5 h, TBS-C1 (3.8 g, 0.025 mol) in DMF (25 mL) was added to the solution at 0° C. Then the reaction mixture was stirred at 20° C. for 6 h. After completion, the reaction mixture was quenched with ice saturated NH₄Cl aq. and extracted with EA. The organic phase was washed with brine, dried over anhydrous Na₂SO₄ and filtered. The filtrate was collected and concentrated. The residue was purified by flash silica gel column chromatography (MeOH/DCM from 0˜10%) to give (6-(((tert-butyldimethylsilyl)oxy)methyl)pyridin-2-yl)methanol (3.2 g, 53%) as white solid.

MS (ESI): mass calcd. for C₁₃H₂₃NO₂Si, 253.2, m/z found 254.2 [M+H]⁺.

### 230-Step 3

To a solution of (6-(((tert-butyldimethylsilyl)oxy)methyl)pyridin-2-yl)methanol (3.2 g, 0.0126 mol) in DCM (30 mL) was added Dess-Martin periodinane (2.9 g, 0.0189 mol). The reaction was stirred at 20° C. for 2 h. After completion, the solvent was concentrated. The residue was purified by flash silica gel column chromatography (PE/EA from 0 50%) to give 6-{[(tert-butyldimethylsilyl)oxy]methyl}pyridine-2-carbaldehyde (3.0 g, 94%) as white oil.

MS (ESI): mass calcd. for C₁₃H₂₁NO₂Si, 251.1, m/z found 252.1 [M+H]⁺.

### 230-Step 4

To a solution of 6-{[(tert-butyldimethylsilyl)oxy]methyl}pyridine-2-carbaldehyde (3.0 g, 0.0119 mol) in DCM (30 mL) was added Et₂NSF₃ (2.9 g, 0.0178 mol) at 0° C. Then the reaction mixture was stirred at 0° C. for 6 h. After completion, the reaction mixture was quenched with water, basified with saturated NaHCO₃aq. and extracted with DCM. The organic phase was washed with brine, dried over anhydrous Na₂SO₄ and filtered. The filtrate was collected and concentrated. The residue was purified by flash silica gel column chromatography (PE/EA from 0˜10%) to give 2-(((tert-butyldimethylsilyl)oxy)methyl)-6-(difluoromethyl)pyridine (1.5 g, 60% pure) as white oil.

MS (ESI): mass calcd. for C₁₃H₂₁F₂NOSi, 273.1, m/z found 274.2 [M+H]⁺.

### 230-Step 5

To a solution of 2-(((tert-butyldimethylsilyl)oxy)methyl)-6-(difluoromethyl)pyridine (1.5 g, 0.0055 mol) in THF (15 mL) was added TBAF (1.58 g, 0.006 mol). The reaction was stirred at 20° C. for 2 h. The solvent was concentrated. The crude was purified by flash silica gel column chromatography (DCM/MeOH from 0˜10%) to give [6-(difluoromethyl)pyridin-2-yl]methanol (0.6 g, 32% two steps) as white oil.

MS (ESI): mass calcd. for C₇H₇F₂NO, 159.1, m/z found 160.1 [M+H]⁺.

### 230-Step 6

To a solution of [6-(difluoromethyl)pyridin-2-yl]methanol (500 mg, 3.14 mmol), 3-hydroxypyridine-4-carbonitrile (453 mg, 3.77 mmol) and PPh₃ (989 mg, 3.77 mmol) in DCM (25 mL) was added DIAD (762 mg, 3.77 mmol) dropwise at 0° C. The reaction mixture was stirred at 0° C. for 1 h. After completion, the solvent was collected and concentrated under reduced pressure and vacuum. The residue was purified by flash silica gel column chromatography (PE/EA from 0˜20%) to give 3-{[6-(difluoromethyl)pyridin-2-yl]methoxy}pyridine-4-carbonitrile (380 mg, 46% yield) as white solid.

MS (ESI): mass calcd. for C₁₃H₉F₂N₃O, 261.1, m/z found 262.2 [M+H]⁺.

### 230-Step 7

To a solution of 3-{[6-(difluoromethyl)pyridin-2-yl]methoxy}pyridine-4-carbonitrile (380 mg, 1.45 mmol) in NH₃-MeOH (7M, 5 mL) and MeOH (20 mL) was added Raney-Ni (380 mg) under N₂. The reaction was stirred at 20° C. for 16 h under H2. After completion, the solvent was collected and concentrated under reduced pressure and vacuum. The residue was purified by flash silica gel column chromatography (DCM/MeOH from 0˜10%) to give (3-{[6-(difluoromethyl)pyridin-2-yl]methoxy}pyridin-4-yl)methanamine (360 mg, 93% yield) as white solid.

MS (ESI): mass calcd. for C₁₃H₁₃F₂N₃O, 265.1, m/z found 266.1 [M+H]⁺.

### 230-Step 8

To a solution of (3-{[6-(difluoromethyl)pyridin-2-yl]methoxy}pyridin-4-yl)methanamine (260 mg, 0.98 mmol), tert-butyl {3-[(3-chloro-2-methoxyphenyl)carbamothioyl]-4-hydroxy-2-oxo-5,6-dihydropyridin-1-yl} formate (527 mg, 1.27 mmol) and PyBOP (2.0 g, 3.92 mmol) in DMA (10 mL) was added DIPEA (1000 mg, 7.84 mmol). The reaction mixture was stirred at 20° C. for 4 h. After completion, the reaction mixture was quenched with water, extracted with EA. The organic phase was washed with brine, dried over anhydrous Na₂SO₄ and filtered. The filtrate was collected and concentrated. The residue was purified by flash silica gel column chromatography (DCM/MeOH from 0˜10%) to give tert-butyl {3-[(3-chloro-2-methoxyphenyl)carbamothioyl]-4-{[(3-{[6-(difluoromethyl)pyridin-2-yl]methoxy}pyridin-4-yl)methyl]amino}-2-oxo-5,6-dihydropyridin-1-yl} formate (280 mg, 48%) as a yellow solid.

### 230-Step 9

To a solution of tert-butyl {3-[(3-chloro-2-methoxyphenyl)carbamothioyl]-4-{[(3-{[6-(difluoromethyl)pyridin-2-yl]methoxy}pyridin-4-yl)methyl]amino}-2-oxo-5,6-dihydropyridin-1-yl} formate (280 mg, 0.42 mmol) in 1,4-dioxane (5.0 mL) was added TFA (97 mg, 0.85 mmol) and H₂O₂ (30%, 144 mg, 1.27 mmol). The reaction was stirred for 1 h at 90° C. After completion, the reaction mixture was cooled to room temperature and concentrated under reduced pressure. The residue was diluted with EA, and the organic phase washed with water, brine, dried over anhydrous Na₂SO₄ and filtered. The filtrate was collected and concentrated. The residue was purified by prep-HPLC with the following conditions: Column: Xbridge 5u C18 150×19 mm; Mobile Phase A: H₂O (0.1% FA), Mobile Phase B: ACN—HPLC; Flow rate: 20 mL/min; Gradient: 27% B to 37% B in 8 min; 214 nm; Rt: 5.63 min to afford 3-((3-chloro-2-methoxyphenyl)amino)-2-(3-((6-(difluoromethyl)pyridin-2-yl)methoxy)pyridin-4-yl)-6,7-dihydro-1H-pyrrolo[3,2-c]pyridin-4(5H)-one (28 mg, 13%) as yellow solid.

MS (ESI): mass calcd. for C₂₆H₂₂ClF₂N₅O₃, 525.1, m/z found 526.1 [M+H]⁺.

¹H NMR (400 MHz, DMSO) δ 11.35 (s, 1H), 8.37 (s, 1H), 8.09-8.02 (m, 2H), 7.68 (d, J=7.6 Hz, 1H), 7.61 (d, J=8.0 Hz, 1H), 7.51 (s, 1H), 7.32 (d, J=4.8 Hz, 1H), 7.13 (s, 0.25H), 7.11 (s, 1H), 7.00 (s, 0.5H), 6.86 (s, 0.25H), 6.67-6.61 (m, 2H), 6.16 (dd, J=5.6, 3.6 Hz, 1H), 5.49 (s, 2H), 3.80 (s, 3H), 3.43 (dt, J=6.8, 2.0 Hz, 2H), 2.87 (t, J=6.8 Hz, 2H).

### Example 231. 3-((3-chloro-2-methoxyphenyl)amino)-2-(3-((3-(trifluoromethyl)pyridin-2-yl)methoxy)pyridin-4-yl)-1,5,6,7-tetrahydro-4H-pyrrolo[3,2-c]pyridin-4-one (compound 540)

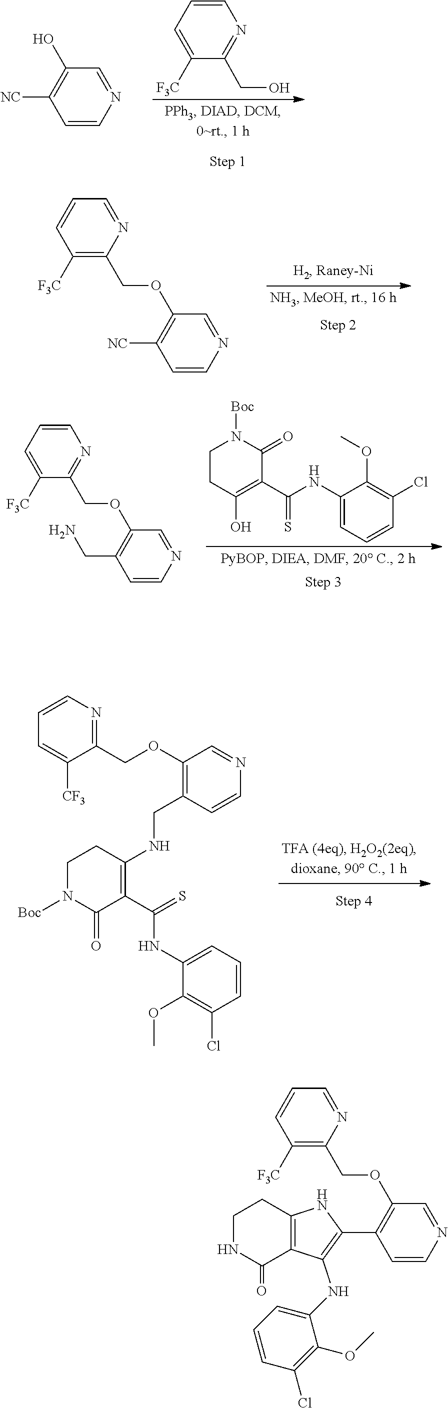

### 231-Step 1

To a solution of 3-hydroxyisonicotinonitrile (416 mg, 3.46 mmol), (3-(trifluoromethyl) pyridin-2-yl) methanol (614 mg, 3.46 mmol) and triphenylphosphine (999 mg, 3.81 mmol) in DCM (10 mL) was added DIAD (771 mg, 3.81 mmol) dropwise at 0° C. The mixture was stirred at 25° C. for 1 hour under nitrogen. After completion, the reaction mixture was quenched with ice saturated NH₄Cl aq, extracted with DCM. The organic phase was washed with brine, dried over anhydrous Na₂SO₄ and filtered. The filtrate was collected and concentrated. The residue was purified by flash silica gel column chromatography (EA/PE from 20%˜50%) to give 3-((3-(trifluoromethyl) pyridin-2-yl) methoxy) isonicotinonitrile (320 mg, 33%) as white solid.

MS (ESI): mass calcd. for C13H₈F₃N₃O, 279.1, m/z found 280.1 [M+H]⁺.

### 231-Step 2

To a stirred solution of 3-((3-(trifluoromethyl) pyridin-2-yl) methoxy) pyridine-4-carbonitrile (320 mg, 1.15 mmol) in MeOH (10 mL) was added Raney-Ni (50 mg) and NH₃·H₂O (1 mL) under N₂. The reaction mixture was stirred at room temperature for 16 h under H2. After completion, the solvent was collected and concentrated under reduced pressure. The residue was purified by flash silica gel column chromatography (MeOH/DCM from 0˜10%) to give (3-((3-(trifluoromethyl) pyridin-2-yl) methoxy)pyridin-4-yl) methanamine (230 mg, 70%) as white solid.

MS (ESI): mass calcd. for C₁₃H₁₂F₃N₃O, 283.1, m/z found 284.1 [M+H]⁺.

### 231-Step 3

To a solution of (3-((3-(trifluoromethyl) pyridin-2-yl) methoxy) pyridin-4-yl) methanamine (230 mg, 0.81 mmol), tert-butyl (3-((3-chloro-2-methoxyphenyl) carbamothioyl)-4-hydroxy-2-oxo-5,6-dihydropyridin-1-yl) formate (403 mg, 0.97 mmol), PYBOP (845 mg, 1.62 mmol) in DMF (10 mL) was added N,N-Diisopropylethylamine (105 mg, 0.81 mmol). The reaction was stirred at 20° C. for 2 h under nitrogen. After completion, the reaction mixture was quenched with water, extracted with EA. The organic phase was washed with brine, dried over anhydrous Na₂SO₄ and filtered. The filtrate was collected and concentrated. The residue was purified by flash silica gel column chromatography (DCM/MeOH from 0˜10%) to give tert-butyl 5-((3-chloro-2-methoxyphenyl) carbamothioyl)-6-oxo-4-(((3-((3-(trifluoromethyl) pyridin-2-yl) methoxy) pyridin-4-yl) methyl) amino)-3,6-dihydropyridine-1(2H)-carboxylate (220 mg, 39%) as yellow oil.

MS (ESI): mass calcd. for C₃₁H₃₁ClF₃N₅O₅S, 677.2, m/z found 678.2 [M+H]⁺.

### 231-Step 4

The solution of tert-butyl (3-((3-chloro-2-methoxyphenyl)carbamothioyl)-2-oxo-4-(((3-((3-(trifluoromethyl)pyridin-2-yl)methoxy)pyridin-4-yl)methyl)amino)-5,6-dihydropyridin-1-yl) formate (220 mg, 0.32 mmol), Hydrogen peroxide (30%, 73 mg, 2.14 mmol) and trifluoroacetic acid (148 mg, 1.29 mmol) in dioxane (10 mL) was stirred at 90° C. for 1 h. After completion, the reaction mixture was cooled to room temperature and quenched with Na₂SO₃ aq. The mixture was extracted with EA, and the organic phase washed with water, brine, dried over anhydrous Na₂SO₄ and filtered. The filtrate was collected and concentrated. The residue was purified by prep-HPLC with the following conditions: Column: Welch 5u C18 150×19 mm; Mobile Phase A: H₂O (0.1% FA), Mobile Phase B: ACN—HPLC; Flow rate: 25 mL/min; Gradient: 32% B to 42% B in 9 min; 214 nm; Rt: 8 min to give 3-((3-chloro-2-methoxyphenyl)amino)-2-(3-((3-(trifluoromethyl)pyridin-2-yl)methoxy)pyridin-4-yl)-1,5,6,7-tetrahydro-4H-pyrrolo[3,2-c]pyridin-4-one (5.5 mg, 3%) as yellow solid.

MS (ESI): mass calcd. for C₂₆H₂₁ClF₃N₅O₃, 543.1, m/z found 544.1 [M+H]⁺.

¹H NMR (400 MHz, CD₃OD) δ 9.06 (d, J=4.8 Hz, 1H), 8.60 (s, 1H), 8.33 (d, J=7.6 Hz, 1H), 8.10 (d, J=6.0 Hz, 1H), 7.80-7.58 (m, 2H), 6.82 (dd, J=8.0, 1.2 Hz, 1H), 6.72 (t, J=8.0 Hz, 1H), 6.30 (dd, J=8.4, 1.2 Hz, 1H), 5.88 (s, 2H), 4.02 (s, 3H), 3.67 (t, J=7.2 Hz, 2H), 3.20 (t, J=6.8 Hz, 2H) Example 232. 3-((3-chloro-2-methoxyphenyl)amino)-2-[3-(pyrazin-2-ylmethoxy)pyridin-4-yl)-1H,5H,6H,7H-pyrrolo(3,2-c)pyridin-4-one (compound 541)

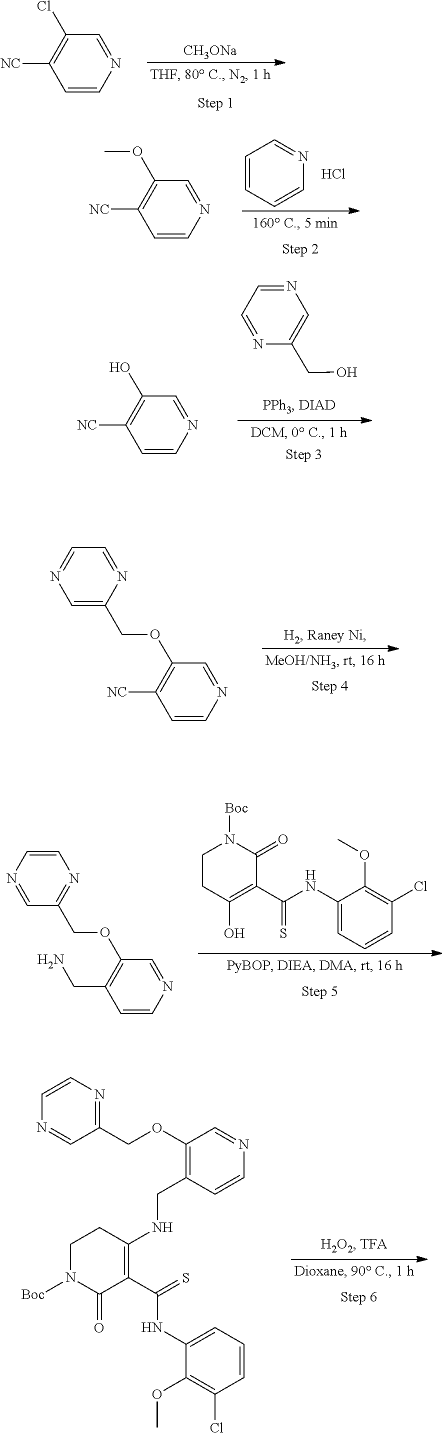

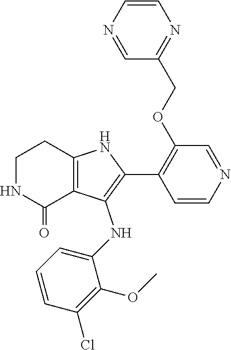 [SMILES: COc1c(Cl)cccc1Nc1c(-c2ccncc2OCc2cnccn2)[nH]c2c1C(=O)NCC2]

### 232-Step 1

To a solution of 3-chloropyridine-4-carbonitrile (10.0 g, 0.072 mol) in dry THF (30 mL) stirred under nitrogen was added sodium methoxide (17.6 g, 0.32 mol) in dry THF (30 mL) dropwise. The reaction mixture was stirred at 80° C. under nitrogen for 1 h. After completion, the reaction mixture was quenched with citric acid (aq.), extracted with EA. The organic phase was washed with brine, dried over anhydrous Na₂SO₄ and filtered. The filtrate was collected and concentrated to afford 3-methoxypyridine-4-carbonitrile (11.0 g, 97%) as a white solid. The crude was used in next step directly without further purification.

MS (ESI): mass calcd. for C₇H₆N₂O, 134.1, m/z found 135.1 [M+H]⁺.

### 232-Step 2

A round-bottom flask containing a mixture of 3-methoxypyridine-4-carbonitrile (6.0 g, 0.0447 mol) and pyridine hydrochloride (18.1 g, 0.16 mol) was placed in oil bath and heated to 160° C. for 5 min under nitrogen. After completion, the reaction mixture was quenched with citric acid (aq.), extracted with EA. The organic phase was washed with brine, dried over anhydrous Na₂SO₄ and filtered. The filtrate was collected and concentrated to afford 3-hydroxypyridine-4-carbonitrile (4.6 g, 81%) as a gray solid.

MS (ESI): mass calcd. for C₆H₄N₂O, 120.1, m/z found 121.1 [M+H]⁺.

### 232-Step 3

To a solution of 3-hydroxypyridine-4-carbonitrile (1.0 g, 0.0082 mol), pyrazin-2-ylmethanol (1.0 g, 0.0090 mol) and Ph₃P (2.4 g, 0.0090 mol) in DCM (20 mL) was added DIAD (1.82 g, 0.0090 mol) in dropwise at 0° C. under nitrogen. The reaction mixture was stirred at 0° C. under nitrogen for 1 h. After completion, the mixture was washed with HCl (aq., 1.0 M). The water phase was neutralized with NaHCO₃(aq.), extracted with EA. the organic phase was washed with brine, dried over anhydrous Na₂SO₄ and filtered. The filtrate was collected and concentrated. The crude was purified by flash chromatography (DCM/MeOH from 0˜10%) to afford 3-(pyrazin-2-ylmethoxy) pyridine-4-carbonitrile (1.9 g, 81%) as a gray solid.

MS (ESI): mass calcd. for C₁₁H₈N₄O, 212.1, m/z found 213.1 [M+H]⁺.

### 232-Step 4

To a solution of 3-(pyrazin-2-ylmethoxy) pyridine-4-carbonitrile (1.0 g, 0.0047 mol), ammonia (7.0 M in MeOH, 15 mL) in MeOH (30 mL) was added Raney Ni (0.8 g). The mixture was stirred at room temperature for 16 h under hydrogen. After completion, the solvent was collected and concentrated under reduced pressure and vacuum. The residue was purified by flash silica gel column chromatography (DCM/MeOH from 0˜10%) to afford (3-(pyrazin-2-ylmethoxy) pyridin-4-yl) methanamine (0.9 g, 79%) as a yellow solid.

MS (ESI): mass calcd. for C₁₁H₁₂N₄O, 216.2, m/z found 217.2 [M+H]⁺.

### 232-Step 5

To a solution of (3-(pyrazin-2-ylmethoxy)pyridin-4-yl]methanamine (223 mg, 1.03 mmol), tert-butyl(3-((3-chloro-2-methoxyphenyl)carbamothioyl)-4-hydroxy-2-oxo-5,6-dihydropyridin-1-yl) formate (640 mg, 1.546 mmol) and DIEA (1.07 mg, 8.25 mmol) in DMA (5 mL) was added PyBOP (2.15 g, 4.12 mmol). The reaction mixture was stirred at room temperature for 16 h under nitrogen. After completion, the reaction mixture was quenched with water, extracted with EA. The organic phase was washed with brine, dried over anhydrous Na₂SO₄ and filtered. The filtrate was collected and concentrated. The residue was purified by flash silica gel column chromatography (DCM/MeOH from 0 10%) to give tert-butyl (3-((3-chloro-2-methoxyphenyl)carbamothioyl)-2-oxo-4-(((3-(pyrazin-2-ylmethoxy)pyridin-4-yl)methyl)amino)-5,6-dihydropyridin-1-yl) formate (950 mg, 45%) as a yellow oil.

MS (ESI): mass calcd. for C₂₉H₃₁ClN₆O₅S, 610.1, m/z found 511.1 [M-100+H]⁺.

### 232-Step 6

To a solution of tert-butyl (3-((3-chloro-2-methoxyphenyl)carbamothioyl)-2-oxo-4-(((3-(pyrazin-2-ylmethoxy)pyridin-4-yl)methyl)amino)-5,6-dihydropyridin-1-yl) formate (950 mg, 0.0016 mol) in 1,4-dioxane (15 mL) was added H₂O₂ (30%, 730 mg, 0.0064 mol) and trifluoroacetic acid (730 mg, 0.0064 mol) dropwise at room temperature under nitrogen atmosphere. The reaction mixture was stirred for 1 h at 90° C. under nitrogen atmosphere. After completion, the reaction mixture was cooled to room temperature and concentrated under reduced pressure. The residue was diluted with EA, and the organic phase washed with water, brine, dried over anhydrous Na₂SO₄ and filtered. The filtrate was collected and concentrated. The residue was purified by prep-HPLC with the following conditions: Column: Welch 10 u C18 250×21.2 mm; Mobile Phase A: Water (0.1% NH₃), Mobile Phase B: ACN; Flow rate: 25 mL/min; Gradient: 35% B to 45% B in 9 min; 214 nm; Rt: 8 min to give 3-((3-chloro-2-methoxyphenyl)amino)-2-[3-(pyrazin-2-ylmethoxy)pyridin-4-yl)-1H,5H,6H,7H-pyrrolo(3,2-c)pyridin-4-one (25 mg, 6%) as a white solid.

MS (ESI): mass calcd. for C₂₄H₂₁ClN₆O₃, 476.1, m/z found 477.1 [M+H]⁺.

¹H NMR (400 MHz, DMSO-d6) δ 11.70 (s, 1H), 8.80 (d, J=1.2 Hz, 1H), 8.74-8.73 (m, 1H), 8.68 (d, J=2.4 Hz, 1H), 8.46 (s, 1H), 8.06 (d, J=4.8 Hz, 1H), 7.51 (s, 1H), 7.34 (d, J=5.2 Hz, 1H), 7.14 (s, 1H), 6.68-6.63 (m, 2H), 6.16-6.14 (m, 1H), 5.56 (s, 2H), 3.84 (s, 3H), 3.45-3.42 (m, 2H), 2.91 (t, J=6.8 Hz, 2H).

### Example 233. 3-((3-chloro-2-methoxyphenyl)amino)-2-(3-(2-(pyrazin-2-yl)ethoxy)pyridin-4-yl)-1,5,6,7-tetrahydro-4H-pyrrolo[3,2-c]pyridin-4-one (compound 542)

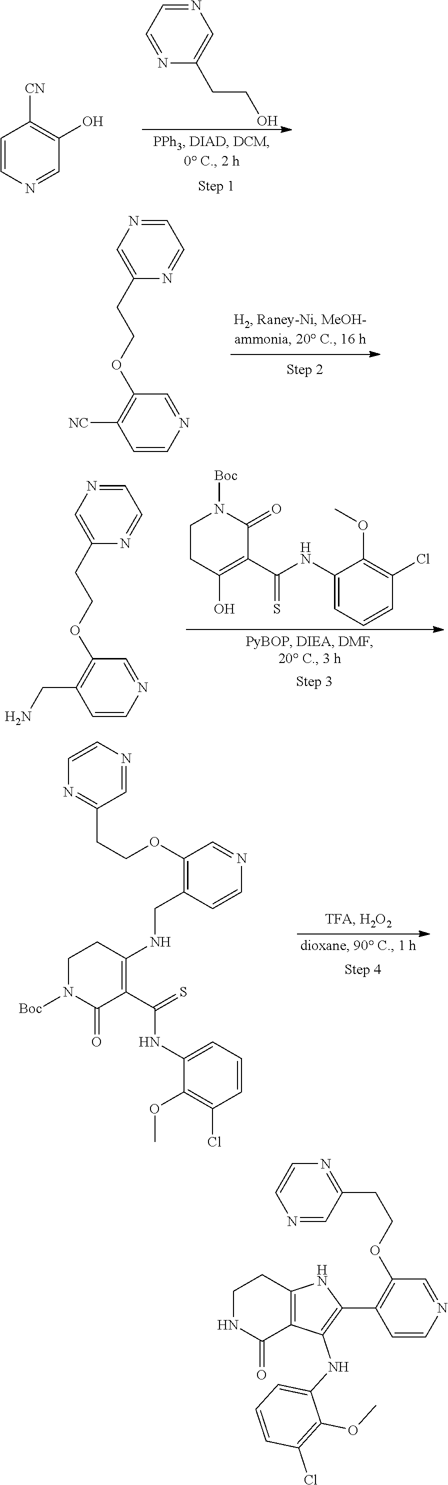

### 233-Step 1

To a solution of 3-hydroxyisonicotinonitrile (0.8 g, 6.66 mmol), 2-(pyrazin-2-yl)ethan-1-ol (0.8 g, 6.66 mmol) and triphenylphosphine (1.9 g, 7.33 mmol) in DCM (10 mL) was added DIAD (1.5 g, 7.33 mmol) dropwise at 0° C. The mixture was stirred at 0° C. for 2 h under nitrogen. After completion, the reaction mixture was quenched with ice saturated NH₄Cl aq, extracted with DCM. The organic phase was washed with brine, dried over anhydrous Na₂SO₄ and filtered. The filtrate was collected and concentrated. The residue was purified by flash silica gel column chromatography (EA/PE from 20%˜50%) to give 3-(2-(pyrazin-2-yl)ethoxy)isonicotinonitrile (1.1 g, 72%) as white solid.

MS (ESI): mass calcd. for C₁₂H₁₀N₄O, 226.1, m/z found 227.1 [M+H]⁺.

### 233-Step 2

To a stirred solution of 3-(2-(pyrazin-2-yl)ethoxy)isonicotinonitrile (1.1 g, 4.82 mmol) in MeOH (100 mL) was added Raney-Ni (500 mg) and NH₃·H₂O (10 mL) under N₂. The reaction mixture was stirred at room temperature for 16 h under H2. After completion, the solvent was collected and concentrated under reduced pressure and vacuum. The residue was purified by flash silica gel column chromatography (MeOH/DCM from 0˜10%) to give (3-(2-(pyrazin-2-yl)ethoxy)pyridin-4-yl)methanamine (850 mg, 76%) as white solid.

MS (ESI): mass calcd. for C₁₄H₁₄N₄O, 230.1, m/z found 231.1 [M+H]⁺.

### 233-Step 3

The solution of (3-(2-(pyrazin-2-yl)ethoxy)pyridin-4-yl)methanamine (850 mg, 3.69 mmol), tert-butyl (3-((3-chloro-2-methoxyphenyl) carbamothioyl)-4-hydroxy-2-oxo-5,6-dihydropyridin-1-yl) formate (1.8 g, 4.43 mmol), PYBOP (3.8 g, 7.38 mmol) and N,N-Diisopropylethylamine (1.0 g, 7.38 mmol) in DMF (15 mL) was stirred at 20° C. for 3 h. After completion, the reaction mixture was quenched with water, extracted with EA. The organic phase was washed with brine, dried over anhydrous Na₂SO₄ and filtered. The filtrate was collected and concentrated. The residue was purified by flash silica gel column chromatography (DCM/MeOH from 0˜10%) to give tert-butyl 5-((3-chloro-2-methoxyphenyl)carbamothioyl)-6-oxo-4-(((3-(2-(pyrazin-2-yl)ethoxy)pyridin-4-yl)methyl)amino)-3,6-dihydropyridine-1(2H)-carboxylate (1.1 g, 48% pure) as a yellow solid.

MS (ESI): mass calcd. for C₃₀H₃₃ClN₆O₅S, 624.2, m/z found 625.2 [M+H]⁺.

### 233-Step 4

The solution of tert-butyl 5-((3-chloro-2-methoxyphenyl)carbamothioyl)-6-oxo-4-(((3-(2-(pyrazin-2-yl)ethoxy)pyridin-4-yl)methyl)amino)-3,6-dihydropyridine-1(2H)-carboxylate (800 mg, 1.28 mmol), Hydrogen peroxide (30%, 290 mg, 2.56 mmol) and trifluoroacetic acid (730 mg, 6.4 mmol) in dioxane (30 mL) was stirred at 90° C. for 1 h. After completion, the reaction mixture was cooled to room temperature and quenched with Na₂SO₃ aq. The mixture was extracted with EA, and the organic phase washed with water, brine, dried over anhydrous Na₂SO₄ and filtered. The filtrate was collected and concentrated. The residue was purified by prep-HPLC with the following conditions: Column: Welch 5u C18 150×19 mm; Mobile Phase A: H₂O (0.1% FA), Mobile Phase B: ACN—HPLC; Flow rate: 25 mL/min; Gradient: 15% B to 35% B in 9.5 min; 214 nm; Rt: 8 min to give 3-((3-chloro-2-methoxyphenyl)amino)-2-(3-(2-(pyrazin-2-yl)ethoxy)pyridin-4-yl)-1,5,6,7-tetrahydro-4H-pyrrolo[3,2-c]pyridin-4-one (21 mg, 3%) as yellow solid.

MS (ESI): mass calcd. for C₂₅H₂₃ClN₆O₃, 490.2, m/z found 491.2 [M+H]⁺.

¹H NMR (400 MHz, DMSO) δ 11.33 (s, 1H), 8.67 (d, J=1.2 Hz, 1H), 8.60-8.59 (m, 1H), 8.52 (d, J=2.4 Hz, 1H), 8.38 (s, 1H), 8.00 (d, J=4.8 Hz, 1H), 7.46 (s, 1H), 7.29 (d, J=4.8 Hz, 1H), 7.13 (s, 1H), 6.71-6.61 (m, 2H), 6.04-6.01 (m, 1H), 4.55 (t, J=6.0 Hz, 2H), 3.87 (s, 3H), 3.45-3.40 (m, 2H), 3.38-3.35 (m, 2H), 2.90 (t, J=6.8 Hz, 2H).

### Example 234. 3-((3-chloro-2-methoxyphenyl)amino)-2-(3-((6-vinylpyrazin-2-yl)methoxy)pyridin-4-yl)-1,5,6,7-tetrahydro-4H-pyrrolo[3,2-c]pyridin-4-one (compound 543)

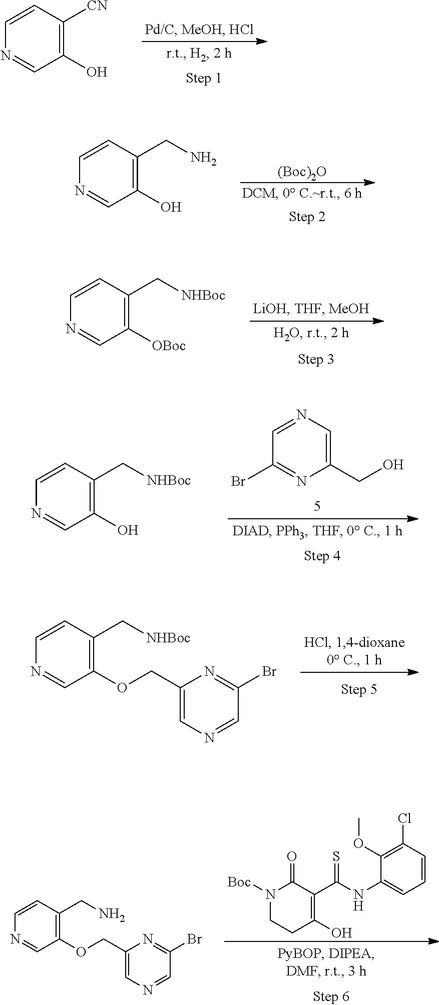

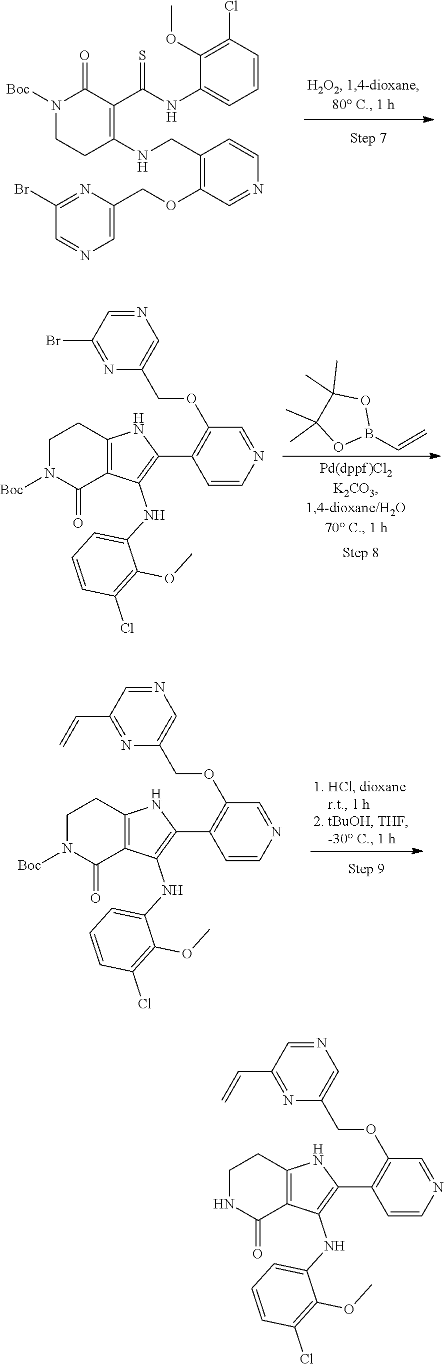

### 234-Step 1

A mixture of 3-hydroxyisonicotinonitrile (1 g, 8.3 mmol) and Pd/C (1 g) in MeOH (60 mL)/HCl (6 mL) was stirred at room temperature for 2 h under hydrogen atmosphere. After completion, the mixture was filtered, washed with MeOH (50 mL). The filtrate was concentrated under reduced pressure. The residue was purified by flash chromatography (DCM/MeOH from 0 to 10%, 0.1% of NH₃₀H as additive) to afford 4-(aminomethyl)pyridin-3-ol (800 mg, yield: 77.7%) as yellow oil.

MS (ESI): mass calcd. for C₆H₈N₂O, 124.06, m/z found 125.2 [M+H]⁺.

### 234-Step 2

To a solution of 4-(aminomethyl)pyridin-3-ol (700 mg, 5.6 mmol) in DCM (35 mL) was added (BOc)₂₀ (1.8 g, 8.4 mmol) at 0° C. and stirred at room temperature for 6 h under N₂. After completion, the reaction was quenched with H₂O (50 mL), extracted with DCM (50 mL×3). The combined organic layers were washed with brine (80 mL), dried over Na₂SO₄, concentrated in vacuo to give the crude, which was purified by flash chromatography (PE/EtOAc from 0 to 60%) to give the tert-butyl ((3-((tert-butoxycarbonyl)oxy)pyridin-4-yl)methyl)carbamate (1.2 g, 66%) as yellow oil.

MS (ESI): mass calcd. for C₁₆H₂₄N₂O₅, 324.17, m/z found 325.2 [M+H]⁺.

### 234-Step 3

A mixture of tert-butyl ((3-((tert-butoxycarbonyl)oxy)pyridin-4-yl)methyl)carbamate (1.2 g, 3.7 mmol) and LiOH—H₂O (466 mg, 11.1 mmol) in MeOH (15 mL)/THF (15 mL)/H₂O (5 mL) was stirred at room temperature for 2 h under N₂. After completion, the mixture was filtered, washed with DCM (50 mL). The filtrate was concentrated under reduced pressure. The residue was purified by flash chromatography (DCM/MeOH from 0 to 10%) to afford tert-butyl ((3-hydroxypyridin-4-yl)methyl)carbamate (800 mg, yield: 96%) as yellow oil.

MS (ESI): mass calcd. for C₁₁H₁₆N₂O₃, 224.12, m/z found 225.2 [M+H]⁺.

### 234-Step 4

To a stirred mixture of tert-butyl ((3-hydroxypyridin-4-yl)methyl)carbamate (600 mg, 2.68 mmol), (6-bromopyrazin-2-yl)methanol (607 mg, 3.2 mmol), and PPh₃ (840 mg, 3.2 mmol) in THF (20 mL) was added DIAD (645 mg, 3.2 mmol) at 0° C. The reaction mixture was stirred at 0° C. for 1 h under N₂. After completion, the reaction was quenched with H₂O (30 mL), extracted with EtOAc (50 mL×3). The combined organic layers were washed with brine (40 mL), dried over Na₂SO₄, filtered and concentrated in vacuo to give the crude, which was purified with silica gel column chromatography (eluent: PE/EtOAc from 0 to 40%) to give tert-butyl ((3-((6-bromopyrazin-2-yl)methoxy)pyridin-4-yl)methyl)carbamate (120 mg, yield: 11%) as a yellow solid.

MS (ESI): mass calcd. for C₁₆H₁₉BrN₄O₃, 394.06, m/z found 395.2 [M+H]⁺.

### 234-Step 5

To a solution of tert-butyl ((3-((6-bromopyrazin-2-yl)methoxy)pyridin-4-yl)methyl)carbamate (120 mg, 0.3 mmol) in 1,4-dioxane (5 mL) was added HCl (0.7 mL, 2.8 mmol, 4M solution in 1,4-dioxane) at 0° C. The resulting reaction mixture was stirred at 0° C. for 1 h. After completed, the reaction mixture was concentrated to give the residue, which was neutralized with NH₃ (10 mL, 7M solution in MeOH) and concentrated. The crude product was purified by Prep-TLC (eluent: DCM/MeOH=10/1) to give (3-((6-bromopyrazin-2-yl)methoxy)pyridin-4-yl)methanamine (75 mg, yield: 85%) as a yellow solid.

MS (ESI): mass calcd. for C₁₁H₁₁BrN₄O, 294.01, m/z found 295.2 [M+H]⁺.

### 234-Step 6

A solution of (3-((6-bromopyrazin-2-yl)methoxy)pyridin-4-yl)methanamine (75 mg, 0.25 mmol), tert-butyl 5-((3-chloro-2-methoxyphenyl)carbamothioyl)-4-hydroxy-6-oxo-3,6-dihydropyridine-1(2H)-carboxylate (80 mg, 0.33 mmol), PyBOP (520 mg, 0.48 mmol) and DIPEA (124 mg, 0.96 mmol) in DMF (5 mL) was stirred at room temperature for 3 h. After completed, the resulting mixture was diluted with water (40 mL), extracted with EtOAc (40 mL×3). The combined organic layers were washed with brine (50 mL), dried over anhydrous Na₂SO₄. After filtration, the filtrate was concentrated under reduced pressure. The residue was purified by flash chromatography (DCM/MeOH from 1 to 5%) to afford tert-butyl 5-((3-chloro-2-methoxyphenyl)carbamothioyl)-6-oxo-4-(((3-(2-(pyridin-2-yl)propoxy)pyridin-4-yl)methyl)amino)-3,6-dihydropyridine-1(2H)-carboxylate (115 mg, yield: 66%) as a yellow solid.

MS (ESI): mass calcd. for C₂₉H₃₀BrClN₆O₅S, 688.09, m/z found 689.1 [M+H]⁺.

### 234-Step 7

To a solution tert-butyl 4-(((3-((6-bromopyrazin-2-yl)methoxy)pyridin-4-yl)methyl)amino)-5-((3-chloro-2-methoxyphenyl)carbamothioyl)-6-oxo-3,6-dihydropyridine-1(2H)-carboxylate (100 mg, 0.14 mmol) in 1,4-dioxane (5 mL) was added H₂O₂ (74.8 mg, 0.66 mmol, 30% solution in H₂O). The resulting mixture was stirred at 80° C. for 3h. The reaction mixture was concentrated to give the crude product, which was purified by Prep-TLC (DCM/MeOH=15/1) to give tert-butyl 2-(3-((6-bromopyrazin-2-yl)methoxy)pyridin-4-yl)-3-((3-chloro-2-methoxyphenyl)amino)-4-oxo-1,4,6,7-tetrahydro-5H-pyrrolo[3,2-c]pyridine-5-carboxylate (50 mg, yield: 54%) as a yellow solid.

MS (ESI): mass calcd. for C₂₉H₂₈BrClN₆O₅, 654.10, m/z found 657.0 [M+H]⁺.

### 234-Step 8

A solution of tert-butyl 2-(3-((6-bromopyrazin-2-yl)methoxy)pyridin-4-yl)-3-((3-chloro-2-methoxyphenyl)amino)-4-oxo-1,4,6,7-tetrahydro-5H-pyrrolo[3,2-c]pyridine-5-carboxylate (50 mg, 0.076 mmol), 4,4,5,5-tetramethyl-2-vinyl-1,3,2-dioxaborolane (23.4 mg, 0.152 mmol) and Pd(dppf)Cl₂ (11.1 mg, 0.015 mmol) and K₂CO₃(32.5 mg, 0.228 mol) in 1,4-dioxane/H₂O (5 mL/1 mL) was stirred at 70° C. for 1 h. After completed, the resulting mixture was diluted with water (40 mL), extracted with EtOAc (40 mL×3). The combined organic layers were washed with brine (50 mL), dried over anhydrous Na₂SO₄. After filtration, the filtrate was concentrated to give the crude product, which was purified by Prep-TLC (DCM/MeOH=15/1) to afford tert-butyl 3-((3-chloro-2-methoxyphenyl)amino)-4-oxo-2-(3-((6-vinylpyrazin-2-yl)methoxy)pyridin-4-yl)-1,4,6,7-tetrahydro-5H-pyrrolo[3,2-c]pyridine-5-carboxylate (35 mg, yield: 76%) as a yellow solid.

MS (ESI): mass calcd. for C₃₁H₃₁ClN₆O₅, 602.20, m/z found 603.3 [M+H]⁺.

### 234-Step 9

A solution of tert-butyl 3-((3-chloro-2-methoxyphenyl)amino)-4-oxo-2-(3-((6-vinylpyrazin-2-yl)methoxy)pyridin-4-yl)-1,4,6,7-tetrahydro-5H-pyrrolo[3,2-c]pyridine-5-carboxylate (15 mg, 0.025 mmol), HCl (0.2 mL, 0.8 mmol, 4 M solution in 1,4-dioxane) was stirred at room temperature for 1 h. The reaction mixture was concentrated to give the crude product, which was re-dissolved in MeOH (4 mL), then tBuOK (2.8 mg, 0.025 mmol) was added thereto at −30° C. for 1 h. After completed, the resulting mixture was diluted with water (20 mL), extracted with EtOAc (30 mL×3). The combined organic layers were washed with brine (30 mL), dried over anhydrous Na₂SO₄. After filtration, the filtrate was concentrated to give the crude product, which was purified by Prep-TLC (DCM/MeOH=15/1) to afford 3-((3-chloro-2-methoxyphenyl)amino)-2-(3-((6-vinylpyrazin-2-yl)methoxy)pyridin-4-yl)-1,5,6,7-tetrahydro-4H-pyrrolo[3,2-c]pyridin-4-one (3.1 mg, yield: 60%) as a yellow solid.

MS (ESI): mass calcd. for C₂₆H₂₃ClN₆O₃, 502.15, m/z found 503.1 [M+H]⁺.

¹H NMR (400 MHz, MeOD) δ 8.64 (s, 1H), 8.59 (s, 1H), 8.39 (s, 1H), 7.99 (d, J=5.2 Hz, 1H), 7.45 (d, J=5.2 Hz, 1H), 6.96-6.88 (m, 1H), 6.65-6.63 (m, 1H), 6.58 (t, J=8.1 Hz, 1H), 6.41 (dd, J=17.5, 1.1 Hz, 1H), 6.17 (dd, J=8.1, 1.5 Hz, 1H), 5.67 (dd, J=10.9, 1.1 Hz, 1H), 5.49 (s, 2H), 3.91 (s, 3H), 3.57 (t, J=6.9 Hz, 2H), 2.94 (t, J=6.9 Hz, 2H).

### Example 235. 3-((3-chloro-2-methoxyphenyl)amino)-2-(3-((6-methylpyrazin-2-yl)methoxy)pyridin-4-yl)-1,5,6,7-tetrahydro-4H-pyrrolo[3,2-c]pyridin-4-one (compound 549)

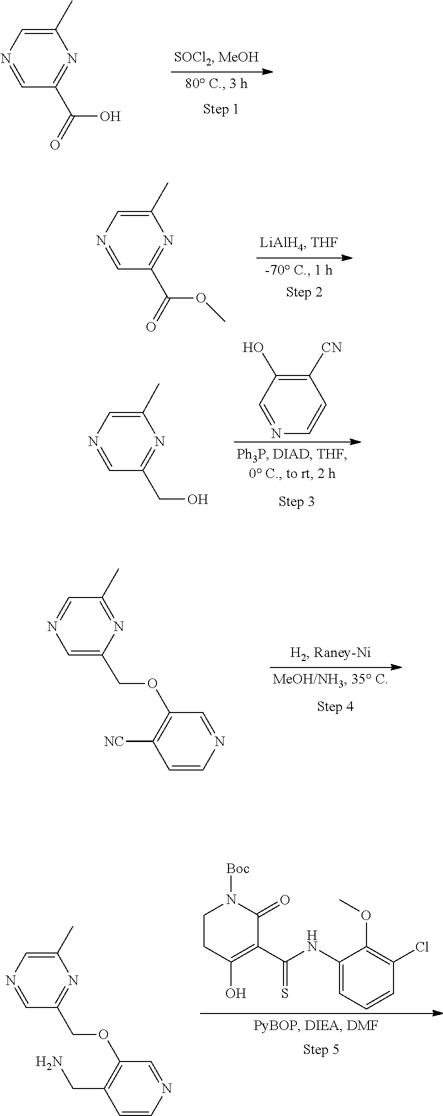

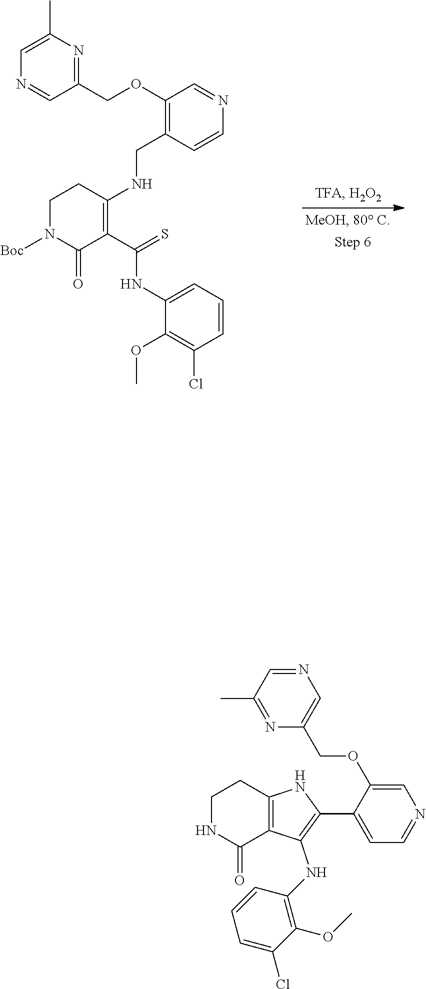 [SMILES: COc1c(Cl)cccc1NC(=S)C1=C(NCc2ccncc2OCc2cncc(C)n2)CCN(C(=O)OC(C)(C)C)C1=O ; COc1c(Cl)cccc1Nc1c(-c2ccncc2OCc2cncc(C)n2)[nH]c2c1C(=O)NCC2]

### 235-Step 1

To a solution of 6-methylpyrazine-2-carboxylic acid (2 g, 0.0145 mol) in MeOH (20 mL) was added sulfurooyl dichloride (3.45 g, 0.029 mol) at 0° C. The reaction mixture was stirred at 80° C. for 6 h. Saturated aqueous NaHCO₃(100 mL) was added and extracted with EA (50 mL×3). The organic layer was dried over anhydrous Na₂SO₄, filtered and concentrated under reduced pressure to give the crude product. The residue was purified by Flash Chromatography to give methyl 6-methylpyrazine-2-carboxylate (1.5 g, 65%) as a yellow solid.

MS (ESI): mass calcd. for C₇H₈N₂O₂, 152.06, m/z found 153.1 [M+H]⁺.

### 235-Step 2

To a solution of methyl 6-methylpyrazine-2-carboxylate (1.5 g, 0.0099 mol) in THF (20 mL) was added LiAlH₄ (0.41 g, 0.0108 mol) at −70° C. The reaction mixture was stirred at −70° C. for 1 h. Na₂SO₄·10H₂O was added. The solution was filtered. The filtrate was dried over anhydrous Na₂SO₄, filtered and concentrated under reduced pressure to give the crude product. The residue was purified by Flash Chromatography to give (6-methylpyrazin-2-yl)methanol (0.6 g, 46%) as a yellow solid.

MS (ESI): mass calcd. for C₆H₈N₂O, 124.6, m/z found 125.1 [M+H]⁺.

### 235-Step 3

To a solution of (6-methylpyrazin-2-yl)methanol (500 mg, 4.0277 mmol), 3-hydroxypyridine-4-carbonitrile (507.95 mg, 4.229 mmol), PPh₃ (1267.71 mg, 4.8332 mmol) in THF (10 mL) stirred under nitrogen at 0° C. was added DIAD (977.33 mg, 4.8332 mmol). The reaction mixture was stirred at room temperature for 3 h. The mixture was diluted with water and extracted with EA (50 mL×3). The organic layer was dried over anhydrous Na₂SO₄, filtered and concentrated under reduced pressure to give the crude product. The residue was purified by Flash Chromatography to give a residue and purified by Flash Chromatography to give 3-((6-methylpyrazin-2-yl)methoxy)isonicotinonitrile (400 mg, 35%) as a yellow solid.

MS (ESI): mass calcd. for C₁₂H₁₀N₄O, 226.09, m/z found 227.0 [M+H]*.

### 235-Step 4

To a solution of 3-[(6-methylpyrazin-2-yl)methoxy]pyridine-4-carbonitrile (400 mg, 1.7681 mmol) in MeOH (10 mL) was added Raney Ni (103.77 mg, 1.7681 mmol) under H2. The reaction mixture was stirred at room temperature under H2 for 6 h. The solution was filtered, filtrate was collected and concentrated in vacuo to give the crude product. The residue was purified by Flash Chromatography to give a residue and purified by Flash Chromatography to give (3-((6-methylpyrazin-2-yl)methoxy)pyridin-4-yl)methanamine (200 mg, 39%) as a yellow solid.

MS (ESI): mass calcd. for C₁₂H₁₄N₄O, 230.12, m/z found 231.1 [M+H]⁺.

### 235-Step 5

To a solution of {3-[(6-methylpyrazin-2-yl)methoxy]pyridin-4-yl}methanamine (200 mg, 0.8686 mmol), tert-butyl {3-[(3-chloro-2-methoxyphenyl)carbamothioyl]-4-hydroxy-2-oxo-5,6-dihydropyridin-1-yl} formate (359.51 mg, 0.8686 mmol) in DMF (10 mL) were added N,N-Diisopropylethylamine (336.77 mg, 2.6058 mmol) and PYBOP (678.02 mg, 1.3029 mmol). The reaction was stirred at room temperature for 3 h. The mixture was diluted with water and extracted with EA (30 mL×3). The organic layer was dried over anhydrous Na₂SO₄, filtered and concentrated under reduced pressure to give the crude product. The residue was purified by Flash Chromatography to give tert-butyl 5-((3-chloro-2-methoxyphenyl)carbamothioyl)-4-(((3-((6-methylpyrazin-2-yl)methoxy)pyridin-4-yl)methyl)amino)-6-oxo-3,6-dihydropyridine-1(2H)-carboxylate (260 mg, 42%) as a yellow solid.

MS (ESI): mass calcd. for C₃₀H₃₃ClN₆O₅S, 624.19, m/z found 625.0/627.0 [M+H]⁺.

### 235-Step 6

To a solution of tert-butyl {3-[(3-chloro-2-methoxyphenyl)carbamothioyl]-4-[({3-[(6-methylpyrazin-2-yl)methoxy]pyridin-4-yl}methyl)amino]-2-oxo-5,6-dihydropyridin-1-yl} formate (260 mg, 0.4152 mmol) in MeOH (10 mL) were added trifluoroacetic acid (94.68 mg, 0.8304 mol) and Hydrogen peroxide (28.25 mg, 0.8304 mol) at 0° C. The reaction was stirred at 80° C. for 3 h. Saturated aqueous Na₂SO₃ was added. The mixture was extracted with EA (30 mL×3). The organic layer was dried over anhydrous Na₂SO₄, filtered and concentrated under reduced pressure to give the crude product. The residue was purified by prep-HPLC to give 3-((3-chloro-2-methoxyphenyl)amino)-2-(3-((6-methylpyrazin-2-yl)methoxy)pyridin-4-yl)-1,5,6,7-tetrahydro-4H-pyrrolo[3,2-c]pyridin-4-one (22.3 mg, 11%) as a yellow solid.

MS (ESI): mass calcd. for C₂₅H₂₃ClN₆O₃, 490.15, m/z found 491.1/493.1 [M+H]⁺.

¹H NMR (400 MHz, DMSO-d6) δ 11.38 (s, 1H), 8.54 (d, J=4.0 Hz, 2H), 8.44 (s, 1H), 8.06 (d, J=4.0 Hz, 1H), 7.50 (s, 1H), 7.32 (d, J=8.0 Hz, 1H), 7.12 (s, 1H), 6.66-6.61 (m, 2H), 6.14 (dd, J=4.0, 8.0 Hz, 1H), 5.46 (s, 2H), 3.81 (s, 3H), 3.45-3.41 (m, 2H), 2.88 (t, J=8.0 Hz, 2H), 2.54 (s, 3H).

### Example 236. 2-(3-{2-[(1S,3R,5S)-2-[(2E)-4-(dimethylamino)but-2-enoyl]-2-azabicyclo[3.1.0]hexan-3-yl]ethynyl}pyridin-4-yl)-3-[(3-fluoro-2-methoxyphenyl)amino]-1H,5H,6H,7H-pyrrolo[3,2-c]pyridin-4-one (compound 345)

### 236.1. Synthesis of tert-butyl (1S,3R,5S)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate

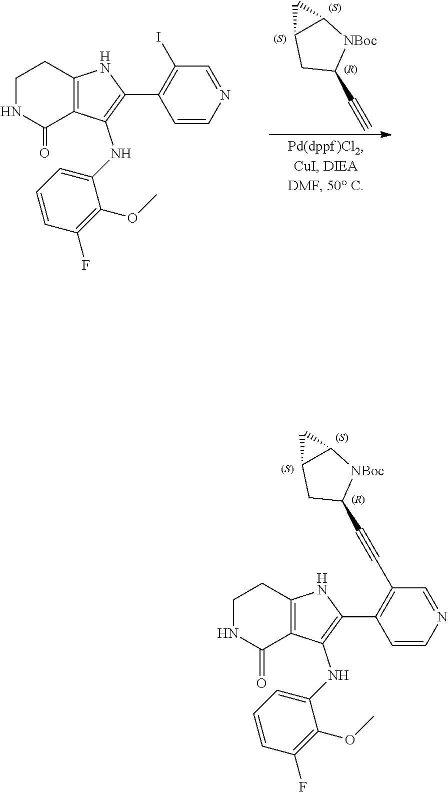

To a stirred mixture of 3-[(3-fluoro-2-methoxyphenyl)amino]-2-(3-iodopyridin-4-yl)-1H,5H,6H,7H-pyrrolo[3,2-c]pyridin-4-one (600 mg, 1.25 mmol, 1.00 equiv) CuI (119 mg, 0.62 mmol, 0.50 equiv) a nd Pd(dppf)Cl₂CH₂Cl₂ (255 mg, 0.31 mmol, 0.25 equiv) in DMF (6 mL) were added tert-butyl (1S,3R,5S)-3-ethynyl-2-azabicyclo[3.1.0]hexane-2-carboxylate (650 mg, 3.13 mmol, 2.50 equiv) and DIEA (486 mg, 3.76 mmol, 3 equiv) dropwise at room temperature under argon atmosphere. The resulting mixture was stirred for 2 h at 50° C. under argon atmosphere. The residue was purified by reverse flash chromatography with the following conditions: column, silica gel; mobile phase, MeCN in water, 10% to 50% gradient in 10 min; detector, UV 254 nm. The resulting mixture was concentrated under reduced pressure. The residue was purified by silica gel column chromatography, eluted with DCM/MeOH (20:1) to afford tert-butyl (1S,3R,5S)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (460 mg, 65.76%) as a yellow solid.

LC-MS: (M+H)⁺ found: 558.20.

### 236.2 Synthesis of 2-(3-{2-[(1S,3R,5S)-2-azabicyclo[3.1.0]hexan-3-yl]ethynyl}pyridin-4-yl)-3-[(3-fluoro-2-methoxyphenyl)amino]-1H,5H,6H,7H-pyrrolo[3,2-c]pyridin-4-one

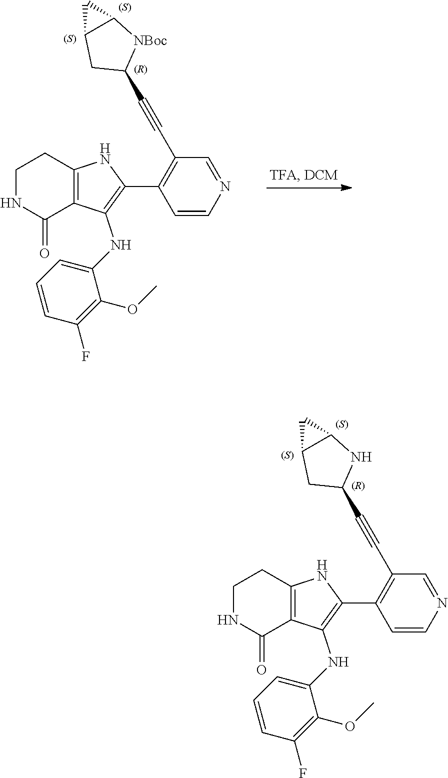

To a stirred mixture of tert-butyl (1S,3R,5S)-3-[2-(4-{3-[(3-fluoro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (100 mg, 0.18 mmol, 1.00 equiv) in DCM (3 mL) was added TFA (1 mL) at room temperature under nitrogen atmosphere. The resulting mixture was stirred for 1 h and concentrated under reduced pressure to afford 2-(3-{2-[(1S,3R,5S)-2-azabicyclo[3.1.0]hexan-3-yl]ethynyl}pyridin-4-yl)-3-[(3-fluoro-2-methoxyphenyl)amino]-1H,5H,6H,7H-pyrrolo[3,2-c]pyridin-4-one (60 mg, crude) as a red oil.

LC-MS: (M+H)⁺ found: 458.10

### 236.3. Synthesis of 3-{2-[(1S,3R,5S)-2-[(2E)-4-(dimethylamino)but-2-enoyl]-2-azabicyclo[3.1.0]hexan-3-yl]ethynyl}pyridin-4-yl)-3-[(3-fluoro-2-methoxyphenyl)amino]-1H,5H,6H,7H-pyrrolo[3,2-c]pyridin-4-one

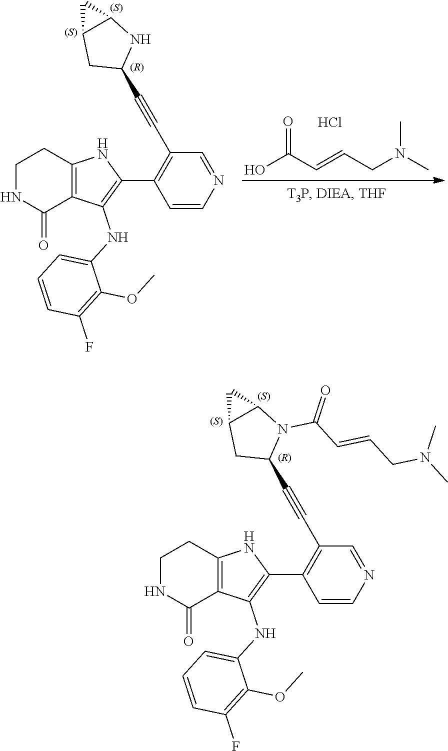

A solution of 2-(3-{2-[(1S,3R,5S)-2-azabicyclo[3.1.0]hexan-3-yl]ethynyl}pyridin-4-yl)-3-[(3-fluoro-2-methoxyphenyl)amino]-1H,5H,6H,7H-pyrrolo[3,2-c]pyridin-4-one (60 mg, 0.13 mmol, 1.00 equiv) in THF (3 mL).was basified to pH 8 with DIEA. To the above mixture was added (2E)-4-(dimethylamino)but-2-enoic acid (33 mg, 0.26 mmol, 2.00 equiv) at 0° C. under nitrogen atmosphere followed by the addition of T₃P (83 mg, 0.26 mmol, 2.00 equiv, 50% in EA) dropwise. The resulting mixture was stirred for 1 h at room temperature under nitrogen atmosphere. Saturated aqueous NaHCO₃(10 mL) was added to the reaction mixture at 0° C. and extracted with EtOAc (3×10 mL), dried over anhydrous Na₂SO₄ and filtered. The filtrate was concentrated under reduced pressure to give crude product (80 mg) that was purified by Prep-HPLC under the following conditions (Column: Xselect CSH F-Phenyl OBD column, 19*250 mm, 5 μm; Mobile Phase A: Water (10 mmol/L NH₄HCO₃), Mobile Phase B: ACN; Flow rate: 25 mL/min; Gradient: 53% B to 83% B in 7 min, 83% B; Wave Length: 254 nm; RT1(min): 6.53; Number Of Runs: 0) to afford 2-(3-{2-[(1S,3R,5S)-2-[(2E)-4-(dimethylamino)but-2-enoyl]-2-azabicyclo[3.1.0]hexan-3-yl]ethynyl}pyridin-4-yl)-3-[(3-fluoro-2-methoxyphenyl)amino]-1H,5H,6H,7H-pyrrolo[3,2-c]pyridin-4-one (25.7 mg, 33.08%) as a yellow solid.

LC-MS: (M+H)⁺ found: 569.15

¹H NMR (400 MHz, Chloroform-d) δ 10.97 (s, 1H), 8.56 (s, 1H), 8.16 (d, J=5.6 Hz, 1H), 7.70 (s, 1H), 7.50-7.44 (m, 1H), 7.03-6.92 (m, 1H), 6.74 (d, J=15.3 Hz, 1H), 6.61-6.51 (m, 1H), 6.50-6.40 (m, 1H), 6.11-6.04 (m, 1H), 5.21 (s, 1H), 4.83-4.75 (m, 1H), 4.09 (d, J=1.3 Hz, 3H), 3.65-3.57 (m, 3H), 3.32 (s, 2H), 3.24 (t, J=6.8 Hz, 2H), 2.66-2.56 (m, 1H), 2.56-2.46 (m, 1H), 2.44 (s, 6H), 2.00 (t, J=7.5 Hz, 1H), 1.14-1.04 (m, 1H), 0.68-0.61 (m, 1H).

### Example 237. Synthesis of 3-[(3-chloro-2-methoxyphenyl)amino]-2-(3-{2-[(1S,3R,5S)-2-[(2E)-4-(dimethylamino)but-2-enoyl]-2-azabicyclo[3.1.0] hexan-3-yl]ethynyl}pyridin-4-yl)-1H,5H,6H,7H-pyrrolo[3,2-c]pyridin-4-one (compound 466)

### 237.1. Synthesis of tert-butyl (1S,3R,5S)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate

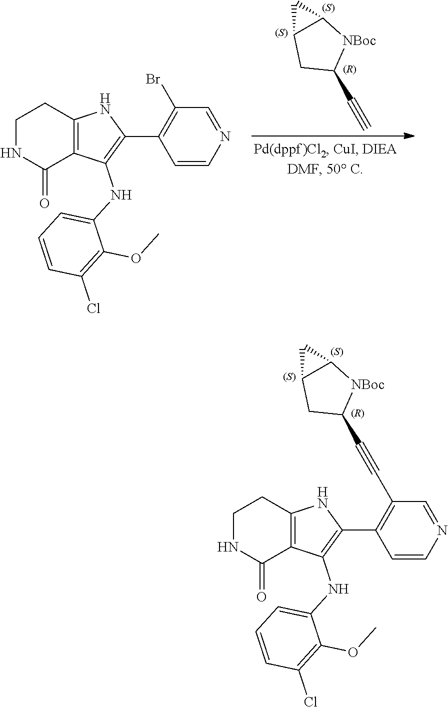

To a stirred mixture of 3-[(3-chloro-2-methoxyphenyl)amino]-2-(3-iodopyridin-4-yl)-1H,5H,6H,7H-pyrrolo[3,2-c]pyridin-4-one (20 mg, 0.040 mmol, 1 equiv) and Pd(dppf)Cl₂CH₂Cl₂ (109.17 mg, 0.134 mmol, 0.25 equiv) CuI (51.05 mg, 0.268 mmol, 0.5 equiv) Bin DMF (2.5 mL) were added tert-butyl (1S,3R,5S)-3-ethynyl-2-azabicyclo[3.1.0]hexane-2-carboxylate (16.76 mg, 0.080 mmol, 2 equiv) and DIEA (207.85 mg, 1.608 mmol, 3 equiv) dropwise at room temperature under argon atmosphere. The resulting mixture was stirred for 2 h at 50° C. under argon atmosphere. The residue was purified by reverse flash chromatography with the following conditions: column, silica gel; mobile phase, MeCN in water, 10% to 50% gradient in 10 min; detector, UV 254 nm. This resulted in tert-butyl (1S,3R,5S)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate as a yellow solid.

LC-MS: (M+H)⁺ found: 574.10.

### 237.2. Synthesis of 2-(3-{2-[(1S,3R,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one

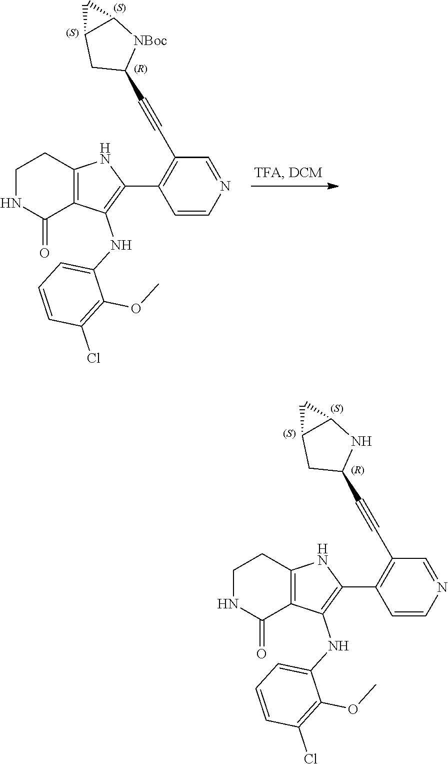

To a stirred mixture of tert-butyl (1S,3R,5S)-3-[2-(4-{3-[(3-chloro-2-methoxyphenyl)amino]-4-oxo-1H,5H,6H,7H-pyrrolo[3,2-c]pyridin-2-yl}pyridin-3-yl)ethynyl]-2-azabicyclo[3.1.0]hexane-2-carboxylate (100 mg, 0.14 mmol, 1.00 equiv) in DCM (3 mL) was added TFA (1 mL) at room temperature under nitrogen atmosphere. The resulting mixture was stirred for 1 h and concentrated under reduced pressure to afford 2-(3-{2-[(1S,3R,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one (80 mg, crude) as a red oil.

LC-MS: (M+H)⁺ found: 474.00

### 237.3. Synthesis of 3-[(3-chloro-2-methoxyphenyl)amino]-2-(3-{2-[(1S,3R,5S)-2-[(2E)-4-(dimethylamino)but-2-enoyl]-2-azabicyclo[3.1.0] hexan-3-yl]ethynyl}pyridin-4-yl)-1H,5H,6H,7H-pyrrolo[3,2-c]pyridin-4-one

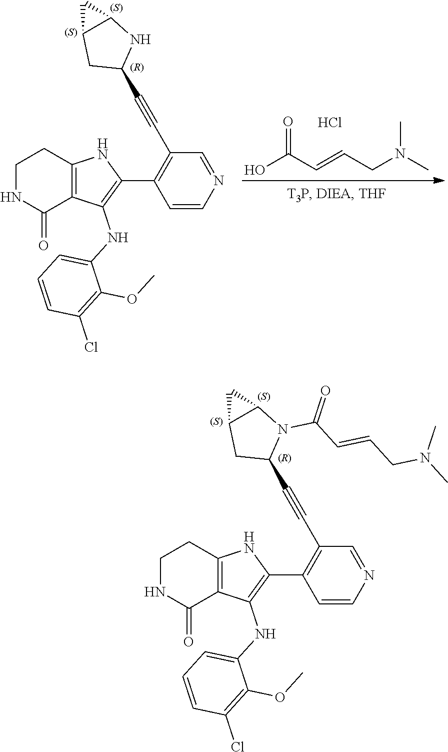

A solution of 2-(3-{2-[(1S,3R,5S)-2-azabicyclo[3.1.0]hexan-3-yl]ethynyl}pyridin-4-yl)-3-[(3-chloro-2-methoxyphenyl)amino]-1H,5H,6H,7H-pyrrolo[3,2-c]pyridin-4-one (80 mg, 0.17 mmol, 1.00 equiv) in THF (3 mL).was basified to pH 8 with DIEA. To the above mixture was added (2E)-4-(dimethylamino)but-2-enoic acid hydrochloride (55 mg, 0.34 mmol, 2.00 equiv) at 0° C. under nitrogen atmosphere followed by the addition of T3P (322 mg, 0.51 mmol, 3 equiv, 50% in EA) dropwise. The resulting mixture was stirred for 1 h at room temperature under nitrogen atmosphere. Saturated aqueous NaHCO₃(10 mL) was added to the reaction mixture at 0° C. and extracted with EtOAc (3×10 mL), dried over anhydrous Na₂SO₄ and filtered. The filtrate was concentrated under reduced pressure to give crude product (140 mg) that was purified by Prep-HPLC under the following conditions (Column: XSelect CSH Fluoro Phenyl, 30*150 mm, 5 μm; Mobile Phase A: Water (10 mmol/L NH₄HCO₃), Mobile Phase B: ACN; Flow rate: 60 mL/min; Gradient: 44% B to 74% B in 7 min; Wave Length: 254 nm; RT1(min): 6.5; Number Of Runs: 0) to afford 3-[(3-chloro-2-methoxyphenyl)amino]-2-(3-{2-[(1S,3R,5S)-2-[(2E)-4-(dimethylamino)but-2-enoyl]-2-azabicyclo[3.1.0] hexan-3-yl]ethynyl}pyridin-4-yl)-1H,5H,6H,7H-pyrrolo[3,2-c]pyridin-4-one (29.1 mg, 23.55%) as a yellow solid.

LC-MS: (M+H)⁺ found: 585.05

¹H NMR (300 MHz, Chloroform-d) δ 10.99 (s, 1H), 8.57 (s, 1H), 8.15 (d, J=5.6 Hz, 1H), 7.71 (s, 1H), 7.41 (d, J=5.5 Hz, 1H), 7.08-6.85 (m, 1H), 6.80-6.49 (m, 3H), 6.31-6.05 (m, 1H), 5.22 (s, 1H), 4.89-4.71 (m, 1H), 4.07 (s, 3H), 3.59 (d, J=7.0 Hz, 3H), 3.24 (t, J=6.7 Hz, 4H), 2.69-2.23 (m, 8H), 2.06-1.93 (m, 1H), 1.14-1.02 (m, 1H), 0.69-0.60 (m, 1H).

### Bioactivity

### Example A. EGFR Activity

Cell lines are generated by transducing Ba/F3 cells with retroviruses containing vectors with EGFR WT, EGFR exon 20 NPG Ins D770_N₇₇₁, EGFR exon 20 ASV Ins V769_D770, EGFR exon 20 SVD Ins D770_N₇₇₁, or EGFR exon 20 FQEA Ins A763_V764 genes and a puromycin selection marker. Transduced cells are selected with puromycin for 7 days and are then be transferred into culture media without Interleukin 3 (IL3). EGFR WT cells are maintained with supplemental EGF. Surviving cells are confirmed to express EGFR by Western blot and maintained as a pool. The IC50 data are included in Table 6.

### Study Design

### 1 Cell Seeding

1.1 Cells are harvested from flask into cell culture medium and the cell number counted.
1.2 Cells are diluted with culture medium to the desired density and 40 μL of cell suspension is added into each well of 384-well cell culture plate and the seeding density is 600 cells/well.

### 2 Compound Preparation and Treatment

2.1 Test compounds are dissolved to 10 mM in a DMSO stock solution. 45 μL of stock solution is transferred to a 384 polypropylene plate (pp-plate). Perform 3-fold, 10-point dilution via transferring 15 μL compound into 30 μL DMSO using a TECAN (EVO200) liquid handler.
2.2 Spin plates at room temperature at 1,000 RPM for 1 minute.
2.3 Transfer 120 nL of diluted compound from compound source plate into the cell plate.
2.4 After compound treatment for 72 hours, perform CTG detection for compound treatment plates as described in “Detection” section.

### 3 Detection

3.1 Plates are removed from incubators and equilibrated at room temperature for 15 minutes.
3.2 Thaw the CellTiter Glo reagents and allow to equilibrate to room temperature before the experiment.
3.3 Add 40 μL of CellTiter-Glo reagent into each well (at 1:1 to culture medium). Then place the plates at room temperature for 30 min followed by reading on EnVision.

### 4 Data Analysis

4.1 Inhibition activity is calculated following the formula below:

% Inhibition=100×(LumHC−LumSample)/(LumHC−LumLC)

where HC is obtained from cells treated with 0.1% DMSO only; and LC is obtained from culture medium only.
4.2 2. Calculate the IC₅₀ by fitting the Curve using Xlfit (v5.3.1.3), equation 201:

Y=Bottom+(Top−Bottom)/(1+10\hat{ }((Log IC₅₀−X)*HillSlope))

The IC50 date are included in Table 4.

### Example B. Inhibitor Activity on EGFR Phosphorylation (pEGFR)

EGFR mutant Ba/F3 cells were generated by transduction with retrovirus containing vectors expressing EGFR exon 20 NPG Ins D770_N₇₇₁, EGFR exon 20 ASV Ins V769_D770, or EGFR exon 20 SVD Ins D770_N₇₇₁ genes along with a puromycin selection marker. Transduced cells are selected with puromycin for 7 days and are then be transferred into culture media without Interleukin 3 (IL3). Surviving cells are confirmed to express EGFR by Western blot and maintained as a pool. CUTO14 cells were obtained from Dr. Robert C. Doebele at the University of Colorado. The IC50 data are included in Table 6.

### Study Design

### 1 Cell Seeding

1.1 Cells are harvested from flask into cell culture medium and the cell number counted.
1.2 Cells are diluted with culture medium to the desired density and 40 μL of cell suspension is added into each well of 384-well cell culture plate and the seeding density is 50K cells/well (Ba/F3) or 12.5K cells/well (CUTO14).

### 2 Compound Preparation and Treatment

2.1 Test compounds are dissolved to 10 mM in a DMSO stock solution. 45 μL of stock solution is transferred to a 384 polypropylene plate (pp-plate). Perform 3-fold, 10-point dilution via transferring 15 μL compound into 30 μL DMSO using a TECAN (EVO200) liquid handler.
2.2 Spin plates at room temperature at 1,000 RPM for 1 minute.
2.3 Transfer 5 nL of diluted compound from compound source plate into the cell plate.
2.4 After compound treatment for 2 hours, perform pEGFR detection by AlphaLISA for compound treatment plates as described in “Detection” section.
   3 Detection by pEGFR AlphaLISA (Perkin-Elmer)
3.1 Plates are removed from incubators and equilibrated at room temperature for 10 minutes, and media was removed
3.2 10 μL of lysis buffer is added and plates shaken at 600 rpm for 1 hr.
3.3 Prepare acceptor mix just before use and dispense 5 μL of acceptor mix to all the wells. Shake 350 rpm for 1 hr in the dark
3.4 Prepare donor mix under low light conditions prior to use. Dispense 5 μL of donor mix to all the wells. Mix well on the shaker, seal and wrap in aluminum foil and incubate 1.5 hrs at room temperature in the dark
3.5 Transfer 18.5 μL mixture to OptiPlate 384, and read using an Envision.

TABLE 6
IC50 Data for EGFR Activity and Inhibitor
Activity on EGFR phosphorylation (pEGFR)¹

| Com- | BaF3 | BaF3 | BaF3 | BaF3 | BaF3 | ASV | SVD | NPG | CUTO14 |
|---|---|---|---|---|---|---|---|---|---|
| pound | ASV | SVD | NPG | FQEA | WT | IC50 | IC50 | IC50 | IC50 |
| # | IC50 | IC50 | IC50 | IC50 | IC50 | (pEGFR) | (pEGFR) | (pEGFR) | (pEGFR) |
| 124 | ++ | ++ | + | + | + | ++ | NA | +++ | NA |
| 128 | NA | NA | + | NA | + | NA | NA | + | NA |
| 130 | + | + | + | + | + | + | NA | NA | NA |
| 131 | NA | NA | + | NA | + | NA | NA | + | NA |
| 133 | NA | NA | ++ | NA | + | NA | NA | +++ | NA |
| 135 | NA | NA | + | NA | + | NA | NA | NA | NA |
| 137 | NA | NA | + | NA | + | NA | NA | ++ | NA |
| 139 | + | + | + | + | + | NA | NA | ++ | NA |
| 140 | +++ | +++ | +++ | +++ | ++ | +++ | +++ | +++ | +++ |
| 149 | NA | NA | ++ | NA | + | NA | NA | NA | NA |
| 150 | ++ | ++ | + | ++ | + | +++ | NA | NA | NA |
| 188 | ++ | ++ | +++ | ++ | +++ | NA | NA | +++ | NA |
| 189 | ++ | ++ | NA | ++ | + | NA | NA | NA | NA |
| 190 | +++ | +++ | +++ | NA | ++ | NA | NA | NA | +++ |
| 191 | ++ | ++ | NA | ++ | + | NA | NA | NA | NA |
| 192 | +++ | +++ | +++ | ++ | ++ | NA | NA | +++ | NA |
| 193 | + | ++ | NA | + | + | NA | NA | NA | NA |
| 194 | + | + | NA | + | + | NA | NA | NA | NA |
| 195 | ++ | ++ | ++ | ++ | + | NA | NA | +++ | NA |
| 196 | + | + | NA | + | + | NA | NA | NA | NA |
| 197 | + | + | NA | + | + | NA | NA | NA | NA |
| 199 | +++ | ++ | NA | +++ | ++ | NA | NA | NA | NA |
| 200 | +++ | +++ | NA | +++ | ++ | NA | NA | NA | NA |
| 201 | +++ | +++ | NA | +++ | ++ | NA | NA | NA | NA |
| 202 | + | + | ++ | NA | + | NA | NA | NA | NA |
| 203 | + | + | + | NA | + | NA | NA | NA | NA |
| 204 | + | + | + | NA | + | NA | NA | NA | NA |
| 205 | + | + | + | NA | + | NA | NA | NA | NA |
| 206 | +++ | +++ | +++ | NA | +++ | NA | NA | NA | +++ |
| 207 | +++ | +++ | +++ | +++ | +++ | +++ | +++ | +++ | +++ |
| 208 | ++ | ++ | +++ | NA | + | NA | NA | +++ | NA |
| 209 | +++ | +++ | +++ | NA | +++ | NA | NA | +++ | NA |
| 210 | +++ | +++ | +++ | NA | + | NA | NA | +++ | NA |
| 211 | +++ | +++ | +++ | NA | +++ | NA | NA | +++ | NA |
| 212 | +++ | +++ | +++ | NA | +++ | NA | NA | +++ | NA |
| 213 | +++ | +++ | +++ | NA | +++ | NA | NA | NA | NA |
| 216 | +++ | +++ | +++ | NA | ++ | NA | NA | NA | NA |
| 217 | +++ | +++ | +++ | NA | ++ | NA | NA | NA | +++ |
| 218 | +++ | +++ | +++ | NA | ++ | NA | NA | NA | NA |
| 219 | ++ | ++ | ++ | NA | + | NA | NA | NA | NA |
| 220 | + | + | ++ | NA | + | NA | NA | NA | NA |
| 221 | ++ | ++ | ++ | NA | + | NA | NA | NA | NA |
| 222 | ++ | ++ | +++ | NA | + | NA | NA | NA | NA |
| 223 | ++ | ++ | +++ | NA | + | NA | NA | NA | NA |
| 224 | +++ | +++ | +++ | NA | ++ | NA | NA | NA | NA |
| 225 | ++ | ++ | ++ | NA | + | NA | NA | NA | NA |
| 226 | +++ | +++ | +++ | NA | ++ | NA | NA | +++ | NA |
| 227 | +++ | +++ | +++ | +++ | ++ | NA | NA | +++ | +++ |
| 228 | ++ | ++ | ++ | NA | + | NA | NA | NA | NA |
| 229 | +++ | +++ | +++ | NA | + | NA | NA | NA | NA |
| 230 | +++ | +++ | +++ | NA | ++ | NA | NA | NA | NA |
| 231 | +++ | +++ | +++ | NA | +++ | NA | NA | NA | NA |
| 232 | +++ | +++ | +++ | +++ | ++ | NA | NA | NA | +++ |
| 233 | +++ | +++ | +++ | +++ | ++ | NA | NA | NA | +++ |
| 234 | +++ | +++ | +++ | NA | ++ | NA | NA | NA | NA |
| 235 | +++ | +++ | +++ | NA | +++ | NA | NA | NA | NA |
| 236 | +++ | +++ | +++ | NA | ++ | NA | NA | NA | +++ |
| 238 | +++ | +++ | +++ | NA | ++ | NA | NA | NA | NA |
| 239 | +++ | +++ | +++ | NA | +++ | NA | NA | NA | NA |
| 240 | +++ | +++ | +++ | NA | +++ | NA | NA | NA | +++ |
| 241 | +++ | +++ | +++ | NA | ++ | NA | NA | NA | NA |
| 242 | +++ | +++ | +++ | NA | ++ | NA | NA | NA | NA |
| 243 | + | + | + | NA | + | NA | NA | NA | NA |
| 244 | + | + | + | NA | + | NA | NA | NA | NA |
| 245 | +++ | +++ | +++ | NA | ++ | NA | NA | +++ | +++ |
| 246 | +++ | +++ | +++ | NA | +++ | NA | NA | +++ | NA |
| 247 | +++ | +++ | +++ | NA | ++ | NA | NA | NA | NA |
| 248 | +++ | +++ | +++ | NA | +++ | NA | NA | NA | NA |
| 249 | +++ | +++ | +++ | NA | ++ | NA | NA | NA | +++ |
| 250 | +++ | +++ | +++ | NA | +++ | NA | NA | NA | NA |
| 251 | +++ | +++ | +++ | NA | ++ | NA | NA | NA | NA |
| 252 | +++ | +++ | +++ | NA | ++ | NA | NA | NA | +++ |
| 253 | +++ | +++ | +++ | NA | +++ | NA | NA | NA | +++ |
| 255 | +++ | +++ | +++ | NA | +++ | NA | NA | NA | +++ |
| 256 | +++ | +++ | +++ | NA | ++ | NA | NA | NA | NA |
| 257 | +++ | +++ | +++ | NA | +++ | NA | NA | NA | +++ |
| 258 | +++ | +++ | +++ | NA | ++ | NA | NA | +++ | +++ |
| 259 | ++ | ++ | + | NA | + | NA | NA | NA | +++ |
| 260 | ++ | ++ | + | NA | + | NA | NA | NA | NA |
| 261 | +++ | +++ | +++ | +++ | ++ | NA | NA | NA | +++ |
| 262 | +++ | +++ | +++ | NA | ++ | NA | NA | NA | NA |
| 263 | +++ | +++ | +++ | NA | ++ | NA | NA | NA | +++ |
| 264 | +++ | +++ | +++ | +++ | ++ | NA | NA | NA | +++ |
| 265 | +++ | +++ | +++ | NA | +++ | NA | NA | NA | +++ |
| 267 | ++ | ++ | ++ | NA | + | NA | NA | NA | NA |
| 268 | ++ | ++ | ++ | NA | ++ | NA | NA | NA | NA |
| 269 | ++ | ++ | ++ | NA | ++ | NA | NA | NA | NA |
| 270 | +++ | +++ | +++ | NA | ++ | NA | NA | NA | +++ |
| 271 | +++ | +++ | +++ | NA | +++ | NA | NA | NA | +++ |
| 272 | +++ | +++ | +++ | NA | +++ | NA | NA | NA | +++ |
| 273 | +++ | +++ | +++ | NA | +++ | NA | NA | NA | +++ |
| 274 | +++ | +++ | +++ | NA | ++ | NA | NA | NA | +++ |
| 276 | +++ | +++ | +++ | NA | +++ | NA | NA | NA | NA |
| 277 | +++ | +++ | +++ | NA | +++ | NA | NA | NA | NA |
| 278 | +++ | +++ | +++ | NA | +++ | NA | NA | NA | NA |
| 279 | +++ | +++ | +++ | NA | +++ | NA | NA | NA | NA |
| 280 | +++ | +++ | +++ | NA | +++ | NA | NA | NA | NA |
| 281 | ++ | +++ | +++ | NA | ++ | NA | NA | NA | NA |
| 282 | ++ | +++ | +++ | NA | ++ | NA | NA | NA | NA |
| 283 | +++ | ++ | +++ | NA | ++ | NA | NA | NA | NA |
| 284 | ++ | +++ | +++ | NA | ++ | NA | NA | NA | NA |
| 285 | +++ | +++ | +++ | NA | ++ | NA | NA | NA | NA |
| 286 | + | + | + | NA | + | NA | NA | NA | NA |
| 287 | +++ | +++ | +++ | NA | ++ | NA | NA | NA | NA |
| 288 | +++ | +++ | +++ | NA | ++ | NA | NA | NA | +++ |
| 289 | +++ | +++ | +++ | NA | ++ | NA | NA | NA | +++ |
| 290 | +++ | +++ | +++ | NA | ++ | NA | NA | NA | NA |
| 291 | +++ | +++ | +++ | NA | ++ | NA | NA | NA | +++ |
| 292 | ++ | ++ | ++ | NA | + | NA | NA | NA | ++ |
| 293 | + | + | + | NA | + | NA | NA | NA | NA |
| 294 | ++ | +++ | +++ | NA | ++ | NA | NA | NA | NA |
| 295 | +++ | +++ | ++ | NA | ++ | NA | NA | NA | NA |
| 296 | +++ | +++ | +++ | NA | +++ | NA | NA | NA | +++ |
| 297 | ++ | ++ | ++ | NA | ++ | NA | NA | NA | NA |
| 298 | ++ | ++ | ++ | NA | + | NA | NA | NA | NA |
| 299 | + | + | + | NA | + | NA | NA | NA | NA |
| 300 | ++ | ++ | ++ | NA | + | NA | NA | NA | NA |
| 301 | +++ | +++ | +++ | NA | ++ | NA | NA | NA | +++ |
| 302 | +++ | +++ | +++ | NA | +++ | NA | NA | NA | +++ |
| 303 | ++ | ++ | ++ | NA | + | NA | NA | NA | NA |
| 304 | +++ | +++ | +++ | NA | +++ | NA | NA | NA | NA |
| 305 | +++ | +++ | +++ | NA | ++ | NA | NA | NA | +++ |
| 306 | ++ | ++ | ++ | NA | + | NA | NA | NA | NA |
| 307 | +++ | +++ | +++ | NA | ++ | NA | NA | NA | +++ |
| 308 | +++ | +++ | +++ | NA | ++ | NA | NA | NA | +++ |
| 309 | +++ | +++ | ++ | NA | ++ | NA | NA | NA | NA |
| 310 | ++ | ++ | ++ | NA | + | NA | NA | NA | NA |
| 311 | +++ | +++ | +++ | NA | ++ | NA | NA | NA | NA |
| 312 | +++ | +++ | +++ | NA | ++ | NA | NA | NA | NA |
| 313 | ++ | ++ | ++ | NA | + | NA | NA | NA | NA |
| 318 | +++ | +++ | +++ | NA | ++ | NA | NA | NA | +++ |
| 319 | +++ | +++ | +++ | NA | ++ | NA | NA | NA | +++ |
| 320 | +++ | +++ | +++ | NA | ++ | NA | NA | NA | NA |
| 321 | +++ | +++ | +++ | NA | ++ | NA | NA | NA | NA |
| 322 | +++ | +++ | +++ | NA | ++ | NA | NA | NA | +++ |
| 323 | +++ | +++ | +++ | NA | ++ | NA | NA | NA | +++ |
| 324 | +++ | +++ | +++ | NA | ++ | NA | NA | NA | NA |
| 325 | +++ | +++ | +++ | NA | ++ | NA | NA | NA | NA |
| 326 | +++ | +++ | +++ | NA | ++ | NA | NA | NA | NA |
| 327 | +++ | +++ | +++ | NA | ++ | NA | NA | NA | NA |
| 329 | +++ | +++ | +++ | NA | +++ | NA | NA | NA | NA |
| 332 | +++ | +++ | +++ | NA | +++ | NA | NA | NA | +++ |
| 333 | +++ | +++ | +++ | NA | +++ | NA | NA | NA | +++ |
| 334 | +++ | +++ | +++ | NA | ++ | NA | NA | NA | NA |
| 335 | +++ | +++ | +++ | NA | ++ | NA | NA | NA | +++ |
| 336 | ++ | ++ | ++ | NA | + | NA | NA | NA | ++ |
| 339 | +++ | +++ | +++ | NA | ++ | NA | NA | NA | NA |
| 340 | + | ++ | ++ | NA | + | NA | NA | NA | NA |
| 343 | +++ | +++ | +++ | NA | ++ | NA | NA | NA | NA |
| 344 | +++ | +++ | +++ | NA | ++ | NA | NA | NA | +++ |
| 345 | +++ | +++ | +++ | NA | ++ | NA | NA | NA | +++ |
| 346 | +++ | +++ | +++ | NA | ++ | NA | NA | NA | +++ |
| 347 | +++ | +++ | +++ | NA | ++ | NA | NA | NA | +++ |
| 348 | +++ | +++ | +++ | NA | ++ | NA | NA | NA | +++ |
| 349 | ++ | +++ | +++ | NA | ++ | NA | NA | NA | NA |
| 350 | +++ | +++ | +++ | NA | ++ | NA | NA | NA | +++ |
| 351 | ++ | +++ | +++ | NA | ++ | NA | NA | NA | NA |
| 352 | ++ | ++ | +++ | NA | ++ | NA | NA | NA | NA |
| 353 | + | + | ++ | NA | + | NA | NA | NA | NA |
| 355 | + | + | ++ | NA | + | NA | NA | NA | NA |
| 356 | +++ | +++ | +++ | NA | ++ | NA | NA | NA | NA |
| 357 | ++ | ++ | ++ | NA | + | NA | NA | NA | ++ |
| 358 | ++ | ++ | ++ | NA | + | NA | NA | NA | ++ |
| 359 | +++ | +++ | +++ | NA | ++ | NA | NA | NA | +++ |
| 361 | +++ | +++ | +++ | NA | ++ | NA | NA | NA | NA |
| 362 | +++ | +++ | +++ | NA | ++ | NA | NA | NA | +++ |
| 363 | +++ | +++ | +++ | NA | +++ | NA | NA | NA | +++ |
| 364 | ++ | +++ | +++ | NA | + | NA | NA | NA | NA |
| 365 | ++ | ++ | ++ | NA | ++ | NA | NA | NA | ++ |
| 366 | +++ | +++ | +++ | NA | ++ | NA | NA | NA | NA |
| 371 | +++ | +++ | +++ | NA | ++ | NA | NA | NA | NA |
| 372 | +++ | +++ | +++ | NA | ++ | NA | NA | NA | NA |
| 373 | +++ | +++ | +++ | NA | +++ | NA | NA | NA | +++ |
| 374 | +++ | +++ | +++ | NA | ++ | NA | NA | NA | +++ |
| 375 | +++ | +++ | +++ | NA | +++ | NA | NA | NA | +++ |
| 376 | +++ | +++ | +++ | NA | +++ | NA | NA | NA | +++ |
| 377 | +++ | +++ | +++ | NA | ++ | NA | NA | NA | NA |
| 382 | +++ | +++ | +++ | NA | ++ | NA | NA | NA | +++ |
| 383 | ++ | ++ | +++ | NA | + | NA | NA | NA | ++ |
| 385 | +++ | +++ | +++ | NA | +++ | NA | NA | NA | +++ |
| 386 | +++ | +++ | +++ | NA | ++ | NA | NA | NA | +++ |
| 387 | +++ | +++ | +++ | NA | ++ | NA | NA | NA | +++ |
| 388 | +++ | +++ | +++ | NA | ++ | NA | NA | NA | +++ |
| 389 | +++ | +++ | +++ | NA | ++ | NA | NA | NA | +++ |
| 390 | +++ | +++ | +++ | NA | ++ | NA | NA | NA | +++ |
| 391 | ++ | ++ | ++ | NA | + | NA | NA | NA | ++ |
| 392 | +++ | +++ | +++ | NA | ++ | NA | NA | NA | +++ |
| 393 | +++ | +++ | +++ | NA | ++ | NA | NA | NA | +++ |
| 394 | ++ | ++ | ++ | NA | + | NA | NA | NA | ++ |
| 395 | ++ | ++ | ++ | NA | + | NA | NA | NA | ++ |
| 400 | +++ | +++ | +++ | NA | ++ | NA | NA | NA | +++ |
| 401 | +++ | +++ | +++ | NA | ++ | NA | NA | NA | +++ |
| 402 | ++ | ++ | ++ | NA | + | NA | NA | NA | NA |
| 406 | +++ | +++ | +++ | NA | +++ | NA | NA | NA | +++ |
| 407 | +++ | +++ | +++ | NA | +++ | NA | NA | NA | +++ |
| 410 | ++ | ++ | +++ | NA | + | NA | NA | NA | NA |
| 411 | ++ | ++ | ++ | NA | + | NA | NA | NA | ++ |
| 416 | ++ | ++ | ++ | NA | + | NA | NA | NA | +++ |
| 417 | ++ | ++ | ++ | NA | + | NA | NA | NA | ++ |
| 418 | ++ | ++ | ++ | NA | + | NA | NA | NA | ++ |
| 419 | ++ | ++ | ++ | NA | + | NA | NA | NA | ++ |
| 420 | +++ | +++ | +++ | NA | ++ | NA | NA | NA | +++ |
| 421 | +++ | +++ | +++ | NA | + | NA | NA | NA | +++ |
| 422 | +++ | +++ | +++ | NA | ++ | NA | NA | NA | +++ |
| 423 | +++ | +++ | +++ | NA | + | NA | NA | NA | +++ |
| 424 | +++ | +++ | +++ | NA | ++ | NA | NA | NA | +++ |
| 425 | +++ | +++ | +++ | NA | ++ | NA | NA | NA | +++ |
| 426 | +++ | +++ | +++ | NA | ++ | NA | NA | NA | +++ |
| 427 | +++ | +++ | +++ | NA | ++ | NA | NA | NA | +++ |
| 428 | +++ | +++ | +++ | NA | ++ | NA | NA | NA | NA |
| 429 | ++ | ++ | +++ | NA | ++ | NA | NA | NA | ++ |
| 430 | +++ | +++ | +++ | NA | ++ | NA | NA | NA | +++ |
| 431 | +++ | +++ | +++ | NA | ++ | NA | NA | NA | +++ |
| 432 | +++ | +++ | +++ | NA | ++ | NA | NA | NA | NA |
| 433 | +++ | +++ | +++ | NA | ++ | NA | NA | NA | +++ |
| 434 | +++ | +++ | +++ | NA | ++ | NA | NA | NA | +++ |
| 435 | +++ | +++ | +++ | NA | ++ | NA | NA | NA | +++ |
| 436 | +++ | +++ | +++ | NA | ++ | NA | NA | NA | +++ |
| 437 | +++ | +++ | +++ | NA | ++ | NA | NA | NA | +++ |
| 438 | +++ | +++ | +++ | NA | ++ | NA | NA | NA | NA |
| 440 | +++ | +++ | +++ | NA | ++ | NA | NA | NA | +++ |
| 441 | +++ | +++ | +++ | NA | ++ | NA | NA | NA | +++ |
| 442 | ++ | ++ | ++ | NA | + | NA | NA | NA | ++ |
| 443 | +++ | +++ | +++ | NA | ++ | NA | NA | NA | +++ |
| 444 | +++ | +++ | +++ | NA | ++ | NA | NA | NA | +++ |
| 445 | ++ | ++ | ++ | NA | + | NA | NA | NA | ++ |
| 446 | +++ | +++ | +++ | NA | ++ | NA | NA | NA | +++ |
| 447 | +++ | +++ | +++ | NA | ++ | NA | NA | NA | +++ |
| 448 | +++ | +++ | +++ | NA | ++ | NA | NA | NA | +++ |
| 449 | +++ | +++ | +++ | NA | ++ | NA | NA | NA | +++ |
| 450 | +++ | +++ | +++ | NA | ++ | NA | NA | NA | +++ |
| 451 | +++ | +++ | +++ | NA | ++ | NA | NA | NA | +++ |
| 452 | +++ | +++ | +++ | NA | ++ | NA | NA | NA | +++ |
| 453 | +++ | +++ | +++ | NA | ++ | NA | NA | NA | +++ |
| 455 | + | + | + | NA | + | NA | NA | NA | NA |
| 456 | +++ | +++ | +++ | NA | ++ | NA | NA | NA | +++ |
| 457 | +++ | +++ | +++ | NA | +++ | NA | NA | NA | +++ |
| 459 | +++ | +++ | +++ | NA | + | NA | NA | NA | NA |
| 460 | +++ | +++ | +++ | NA | ++ | NA | NA | NA | NA |
| 461 | +++ | +++ | +++ | NA | ++ | NA | NA | NA | NA |
| 462 | + | + | + | NA | + | NA | NA | NA | + |
| 463 | ++ | ++ | +++ | NA | + | NA | NA | NA | +++ |
| 464 | + | ++ | + | NA | + | NA | NA | NA | ++ |
| 465 | +++ | +++ | +++ | NA | ++ | NA | NA | NA | +++ |
| 466 | +++ | +++ | +++ | NA | ++ | NA | NA | NA | +++ |
| 467 | +++ | +++ | +++ | +++ | +++ | +++ | +++ | +++ | +++ |
| 468 | ++ | ++ | ++ | ++ | ++ | NA | NA | +++ | NA |
| 469 | NA | NA | +++ | NA | +++ | NA | NA | +++ | NA |
| 470 | + | + | + | + | + | NA | NA | ++ | NA |
| 471 | NA | NA | + | NA | + | NA | NA | + | NA |
| 472 | +++ | +++ | +++ | +++ | +++ | NA | NA | +++ | NA |
| 473 | +++ | +++ | NA | ++ | + | NA | NA | NA | NA |
| 474 | ++ | +++ | + | ++ | ++ | +++ | NA | NA | NA |
| 475 | ++ | ++ | ++ | ++ | ++ | +++ | NA | +++ | NA |
| 476 | +++ | +++ | +++ | +++ | ++ | NA | NA | +++ | NA |
| 477 | NA | NA | +++ | NA | +++ | +++ | NA | +++ | NA |
| 478 | +++ | +++ | +++ | +++ | ++ | +++ | NA | +++ | NA |
| 479 | NA | NA | + | NA | + | NA | NA | ++ | NA |
| 480 | + | + | + | + | + | ++ | NA | +++ | NA |
| 481 | +++ | ++ | + | ++ | ++ | +++ | NA | NA | NA |
| 482 | NA | NA | ++ | NA | + | NA | NA | +++ | NA |
| 483 | ++ | ++ | +++ | ++ | ++ | +++ | NA | +++ | NA |
| 484 | NA | NA | + | NA | + | NA | NA | + | NA |
| 485 | +++ | +++ | NA | +++ | ++ | NA | NA | NA | NA |
| 486 | ++ | +++ | NA | ++ | + | NA | NA | NA | NA |
| 487 | +++ | +++ | +++ | +++ | ++ | +++ | +++ | +++ | +++ |
| 488 | +++ | +++ | +++ | ++ | ++ | +++ | NA | NA | NA |
| 489 | ++ | ++ | NA | ++ | + | NA | NA | NA | NA |
| 490 | ++ | ++ | NA | ++ | + | +++ | NA | NA | NA |
| 491 | +++ | +++ | NA | +++ | ++ | NA | NA | NA | NA |
| 492 | +++ | +++ | +++ | NA | ++ | NA | NA | +++ | +++ |
| 493 | +++ | +++ | +++ | ++ | + | NA | NA | NA | NA |
| 494 | +++ | +++ | +++ | +++ | + | NA | NA | +++ | NA |
| 495 | +++ | +++ | +++ | +++ | ++ | NA | NA | NA | NA |
| 496 | +++ | +++ | +++ | ++ | + | NA | NA | +++ | NA |
| 497 | +++ | +++ | NA | +++ | ++ | NA | NA | NA | NA |
| 498 | ++ | ++ | NA | ++ | + | NA | NA | NA | NA |
| 499 | +++ | +++ | +++ | ++ | + | NA | NA | NA | NA |
| 500 | ++ | ++ | NA | ++ | + | NA | NA | NA | NA |
| 501 | +++ | +++ | NA | +++ | ++ | NA | NA | NA | NA |
| 502 | ++ | ++ | NA | + | + | NA | NA | NA | NA |
| 503 | ++ | ++ | NA | ++ | ++ | NA | NA | NA | NA |
| 504 | ++ | ++ | NA | ++ | + | NA | NA | NA | NA |
| 505 | + | + | NA | + | + | NA | NA | NA | NA |
| 506 | ++ | ++ | NA | ++ | + | NA | NA | NA | NA |
| 507 | ++ | ++ | NA | ++ | + | NA | NA | NA | NA |
| 508 | ++ | ++ | NA | ++ | + | NA | NA | NA | NA |
| 509 | ++ | ++ | NA | ++ | + | NA | NA | NA | NA |
| 510 | +++ | +++ | ++ | NA | ++ | NA | NA | NA | NA |
| 511 | +++ | +++ | +++ | NA | ++ | NA | NA | NA | +++ |
| 512 | +++ | +++ | +++ | +++ | ++ | NA | NA | NA | +++ |
| 513 | +++ | +++ | +++ | +++ | +++ | NA | NA | NA | +++ |
| 514 | +++ | +++ | +++ | NA | ++ | NA | NA | +++ | NA |
| 515 | +++ | +++ | +++ | NA | ++ | NA | NA | +++ | NA |
| 516 | +++ | +++ | +++ | NA | +++ | NA | NA | NA | NA |
| 517 | +++ | +++ | +++ | +++ | ++ | NA | NA | +++ | +++ |
| 518 | +++ | +++ | +++ | +++ | ++ | NA | NA | NA | +++ |
| 519 | +++ | +++ | +++ | +++ | ++ | +++ | +++ | +++ | +++ |
| 520 | +++ | +++ | NA | +++ | +++ | NA | NA | NA | NA |
| 521 | +++ | ++ | NA | +++ | ++ | NA | NA | NA | NA |
| 522 | +++ | ++ | NA | ++ | ++ | NA | NA | NA | NA |
| 523 | +++ | +++ | +++ | NA | + | NA | NA | +++ | NA |
| 524 | ++ | ++ | NA | ++ | + | NA | NA | NA | NA |
| 525 | +++ | +++ | NA | ++ | + | NA | NA | NA | NA |
| 526 | +++ | +++ | +++ | ++ | ++ | NA | NA | NA | NA |
| 527 | +++ | +++ | +++ | +++ | ++ | NA | NA | NA | NA |
| 528 | +++ | +++ | +++ | +++ | ++ | +++ | +++ | +++ | +++ |
| 529 | +++ | +++ | +++ | +++ | ++ | NA | NA | NA | NA |
| 530 | +++ | +++ | +++ | +++ | +++ | NA | NA | NA | NA |
| 531 | +++ | +++ | NA | +++ | ++ | NA | NA | NA | NA |
| 532 | ++ | ++ | NA | ++ | + | NA | NA | NA | NA |
| 533 | +++ | +++ | NA | +++ | ++ | NA | NA | NA | NA |
| 534 | ++ | ++ | NA | ++ | + | NA | NA | NA | NA |
| 535 | +++ | +++ | NA | ++ | + | NA | NA | NA | NA |
| 536 | ++ | ++ | NA | ++ | + | NA | NA | NA | NA |
| 537 | +++ | +++ | NA | ++ | ++ | NA | NA | NA | NA |
| 538 | +++ | +++ | NA | ++ | ++ | NA | NA | NA | NA |
| 539 | +++ | +++ | NA | +++ | ++ | NA | NA | NA | NA |
| 540 | ++ | ++ | NA | ++ | + | NA | NA | NA | NA |
| 541 | +++ | +++ | NA | +++ | ++ | NA | NA | NA | NA |
| 542 | ++ | ++ | NA | ++ | + | NA | NA | NA | NA |
| 543 | +++ | +++ | NA | +++ | ++ | NA | NA | NA | NA |
| 544 | +++ | +++ | NA | +++ | +++ | NA | NA | NA | NA |
| 545 | ++ | ++ | NA | ++ | + | NA | NA | NA | NA |
| 546 | +++ | +++ | +++ | +++ | ++ | +++ | +++ | +++ | +++ |
| 547 | ++ | ++ | NA | + | + | NA | NA | NA | NA |
| 548 | ++ | ++ | NA | ++ | + | NA | NA | NA | NA |
| 549 | +++ | +++ | NA | +++ | ++ | NA | NA | NA | NA |
| 550 | + | + | + | + | + | NA | NA | NA | + |
| 551 | + | + | + | + | + | NA | NA | NA | + |
| 552 | ++ | ++ | NA | ++ | + | NA | NA | NA | NA |
| 553 | +++ | ++ | NA | ++ | + | NA | NA | NA | NA |
| 554 | ++ | ++ | NA | ++ | + | NA | NA | NA | NA |
| 555 | ++ | ++ | NA | ++ | + | NA | NA | NA | NA |
| 556 | +++ | +++ | NA | +++ | ++ | NA | NA | NA | NA |
| 557 | +++ | +++ | NA | +++ | ++ | NA | NA | NA | NA |
| 558 | +++ | +++ | NA | +++ | ++ | NA | NA | NA | NA |
| 559 | +++ | +++ | NA | ++ | ++ | NA | NA | NA | NA |
| 560 | +++ | +++ | NA | +++ | +++ | NA | NA | NA | NA |
| 561 | +++ | +++ | NA | ++ | ++ | NA | NA | NA | NA |
| 562 | ++ | +++ | NA | ++ | + | NA | NA | NA | NA |
| 563 | +++ | +++ | NA | +++ | ++ | NA | NA | NA | NA |
| 564 | ++ | ++ | NA | ++ | + | NA | NA | NA | NA |
| 565 | ++ | ++ | ++ | NA | + | NA | NA | NA | ++ |
| 566 | + | + | ++ | NA | + | NA | NA | NA | + |
| 567 | ++ | +++ | +++ | NA | ++ | NA | NA | NA | +++ |
| 568 | +++ | +++ | +++ | NA | ++ | NA | NA | NA | +++ |
| 569 | +++ | +++ | +++ | NA | ++ | NA | NA | NA | +++ |
| 574 | +++ | +++ | +++ | NA | +++ | NA | NA | NA | NA |
| 575 | +++ | +++ | +++ | NA | +++ | NA | NA | NA | NA |
| 581 | +++ | +++ | +++ | NA | ++ | NA | NA | NA | NA |
| 582 | +++ | +++ | +++ | NA | + | NA | NA | NA | NA |
| 593 | +++ | +++ | +++ | NA | ++ | NA | NA | NA | NA |
| 594 | +++ | +++ | +++ | NA | + | NA | NA | NA | NA |
| 595 | ++ | ++ | +++ | NA | + | NA | NA | NA | NA |
| 603 | ++ | ++ | ++ | NA | + | NA | NA | NA | NA |
| 630 | +++ | +++ | +++ | NA | + | NA | NA | NA | NA |
| 631 | +++ | +++ | +++ | NA | ++ | NA | NA | NA | NA |
| 632 | +++ | +++ | +++ | NA | ++ | NA | NA | NA | NA |
| 634 | +++ | +++ | +++ | NA | ++ | NA | NA | NA | NA |
| 138a | +++ | +++ | +++ | +++ | ++ | NA | NA | +++ | NA |
| 138b | +++ | +++ | +++ | ++ | ++ | NA | NA | +++ | NA |
| 141a | NA | NA | + | NA | + | NA | NA | ++ | NA |
| 141b | NA | NA | + | NA | + | NA | NA | +++ | NA |
| 142a | NA | NA | ++ | NA | ++ | NA | NA | NA | NA |
| 142b | +++ | +++ | +++ | ++ | ++ | NA | NA | +++ | NA |
| 145a | +++ | +++ | +++ | +++ | ++ | +++ | +++ | +++ | +++ |
| 145b | +++ | +++ | +++ | +++ | +++ | +++ | +++ | +++ | +++ |
| 147a | +++ | +++ | +++ | +++ | +++ | +++ | +++ | +++ | +++ |
| 147b | +++ | +++ | +++ | +++ | ++ | +++ | +++ | +++ | +++ |
| 148a | ++ | ++ | NA | ++ | + | NA | NA | NA | NA |
| 148b | ++ | ++ | NA | ++ | + | NA | NA | NA | NA |

¹“+++” indicates that IC50 < 100 nM;
“+” indicates that 100 nM <= IC50 < 1000 nM;
“+” indicates that IC50 >= 1000 nM.
“NA” indicates that the IC50 data is not available for this compound;

What is claimed is:

1. A compound of Formula (I): 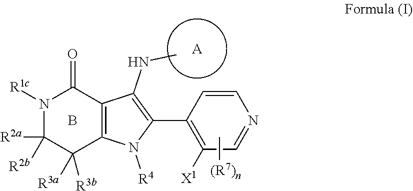
or a pharmaceutically acceptable salt thereof, wherein:
X¹ is selected from the group consisting of: (a) —O-L¹-R⁵; and (b) 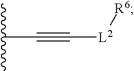
L¹ and L² are independently selected from the group consisting of: a bond and C₁₋₁₀ alkylene optionally substituted with from 1-6 R^{a};
R⁵ is selected from the group consisting of:
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
C₆₋₁₀ aryl optionally substituted with from 1-4 R^{c};
C₃₋₁₀ cycloalkyl or C3-10 cycloalkenyl, each optionally substituted with from 1-4 substituents each independently selected from the group consisting of: oxo and R^{c}; 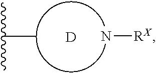
wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), 0, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a};
—R^{W}
—R^{g2}—R^{W} or —R^{g2}—R^{Y};
L⁵-R^{g}; and
L5-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{g}; -L⁵-R^{g}; -L⁵-R^{g2}—R^{W}; or -L5-R^{g2}—R^{Y};
R⁶ is selected from the group consisting of:
H;
halo;
—OH;
—NR^{e}R^{f};
—R^{g};
—R^{W}
-L⁶-R^{g};
—R^{g2}—R^{W} or —R^{g2}—R^{Y};
-L⁶-R^{g2}—R^{W} or -L⁶-R^{g2}—R^{Y}; and
—C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a};
R^{Y} is selected from the group consisting of: —R^{g} and -(L^{g})_{g}-R^{g};
each of R^{1c}, R^{2a}, R^{2b}, R^{3a}, and R^{3b} is independently selected from the group consisting of: H; halo; —OH; —C(O)OH or —C(O)NH₂; —CN; —R^{b}-L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g}; provided that R^{1c} is other than halo, —CN, or —C(O)OH; or
two of variables R^{1c}, R^{2a}, R^{2b}, R^{3a}, and R^{3b}, together with the Ring B ring atoms to which each is attached, form a fused saturated or unsaturated ring of 3-12 ring atoms;
wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W};
Ring A is R^{g};
R⁴ is selected from the group consisting of: H and R^{d},
each R⁷ is an independently selected R^{c}; n is 0, 1, 2, or 3;
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*;
C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C1-10 alkyl which is optionally substituted with from 1-6 independently selected R^{a}; C₃₋₅ cycloalkyl; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₃₋₅ cycloalkyl optionally substituted with from 1-3 C₁₋₃ alkyl group; heterocyclyl including from 3-6 ring atoms, wherein from 1-3 ring atoms are heteroatoms, each independently selected from the group consisting of N, N(H), N(R^{d}), O, and S(O)₀₋₂ optionally substituted with from 1-4 substituents independently selected from the group consisting of oxo and R^{c}; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
C₃₋₁₀ cycloalkyl or C₃₋₁₀ cycloalkenyl, each of which is optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c};
each occurrence of L9 is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and
each occurrence of R′ and R″ is independently selected from the group consisting of: H; —OH; and C₁₋₄ alkyl;
provided that when R^{2a}, R^{2b}, R^{3a}, and R^{3b} are each H; R^{1c} is H or methyl; Ring A is phenyl optionally substituted with from 1-2 F; X¹ is —O-L¹-R⁵; and -L¹ is CH₂, then:
R⁵ is other than unsubstituted phenyl or unsubstituted cyclopropyl; and
further provided that the compound is other than: 3-((3-fluoro-2-methoxyphenyl)amino)-2-(3-((1-phenylpropan-2-yl)oxy)pyridin-4-yl)-1,5,6,7-tetrahydro-4H-pyrrolo[3,2-c]pyridin-4-one.

2. The compound of claim 1, wherein X¹ is —O-L¹-R⁵.

3. The compound of claim 1 or 2, wherein R⁵ is heteroaryl including from 5-ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

4. The compound of any one of claims 1-3, wherein R⁵ is a monocyclic heteroaryl including from 5-6 ring atoms, wherein from 1-4 ring atoms are heteroatoms, each independently selected from the group consisting of N, N(H), N(R^{d}), O, and S, and wherein the heteroaryl is optionally substituted with from 1-4 R^{cA}, wherein each R^{cA} is an independently selected R^{c}.

5. The compound of any one of claims 1-4, wherein R⁵ is monocyclic heteroaryl including 5 ring atoms, wherein from 1-4 ring atoms are heteroatoms, each independently selected from the group consisting of N, N(H), N(R^{d}), O, and S, and wherein the heteroaryl is optionally substituted with from 1-4 R^{cA}, wherein each R^{cA} is an independently selected R^{c}.

6. The compound of any one of claims 1-5, wherein R⁵ is selected from the group consisting of furanyl, thiophenyl, oxadiazolyl, thiadiazolyl, triazolyl, tetrazolyl, imidazolyl, pyrazolyl, oxazolyl, and thiazolyl, each of which is optionally substituted with from 1-2 R^{cA}, and a ring nitrogen is optionally substituted with R^{d}, wherein each R^{cA} is an independently selected R^{c}.

7. The compound of any one of claims 1-6, wherein R⁵ is selected from the group consisting of: 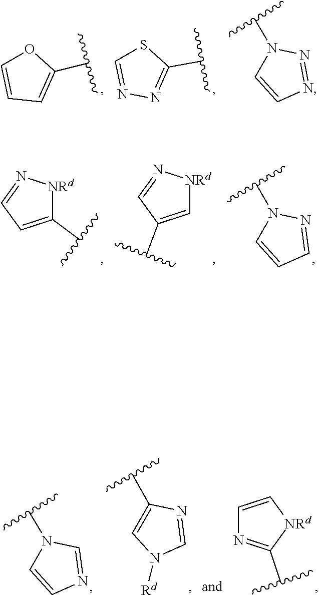
each optionally substituted with from 1-2 R^{cA}, wherein each R^{cA} is an independently selected R^{c}.

8. The compound of any one of claims 1-4, wherein R⁵ is monocyclic heteroaryl including 6 ring atoms, wherein from 1-4 ring atoms are ring nitrogen atoms, and wherein the heteroaryl is optionally substituted with from 1-4 R^{cA}, wherein each R^{cA} is an independently selected R^{c}.

9. The compound of any one of claims 1-4 or 8, wherein R⁵ is selected from the group consisting of pyridyl, pyridonyl, pyrimidyl, pyrazinyl, and pyridazinyl, each optionally substituted with from 1-3 R^{cA}, wherein each R^{cA} is an independently selected R^{c}.

10. The compound of any one of claims 1-4 or 8-9, wherein R⁵ is selected from the group consisting of: 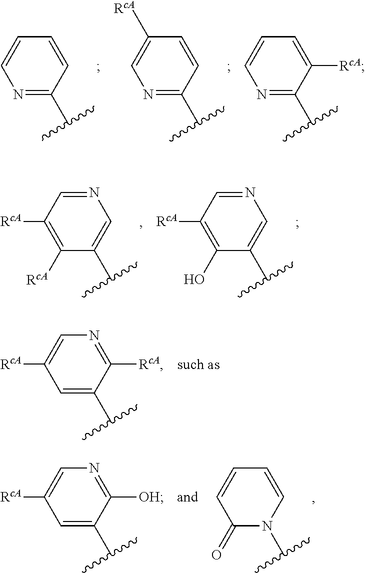
such as each of which is further optionally substituted with R^{cA}, wherein each R^{cA} is an independently selected R^{c}.

11. The compound of any one of claims 1-4 or 8-9, wherein R⁵ is selected from the group consisting of: 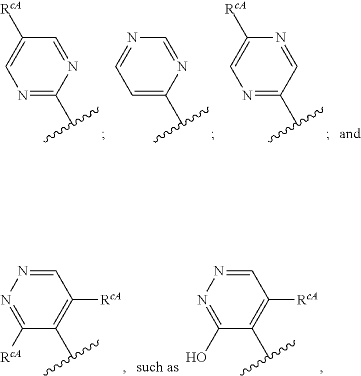
each of which is further optionally substituted with R^{cA}, wherein each R^{cA} is an independently selected R^{c}.

12. The compound of any one of claims 1-3, wherein R⁵ is bicyclic heteroaryl including from 8-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

13. The compound of any one of claims 1-3 or 12, wherein R⁵ is bicyclic heteroaryl including 8 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

14. The compound of any one of claims 1-3 or 12-13, wherein R⁵ is selected from the group consisting of: 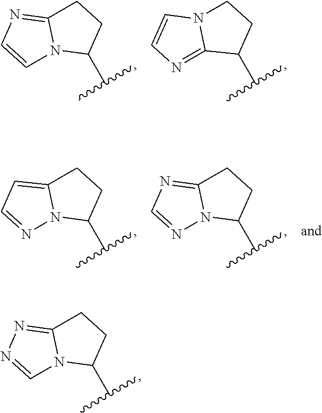 [SMILES: *C1CCc2nccn21 ; *C1CCn2ccnc21 ; *C1CCc2ccnn21 ; *C1CCc2ncnn21 ; *C1CCc2nncn21]
each of which is further optionally substituted with from 1-2 R^{cA}, wherein each R^{cA} is an independently selected R^{c}.

15. The compound of any one of claims 1-2 or 12-13, wherein R⁵ is selected from the group consisting of: 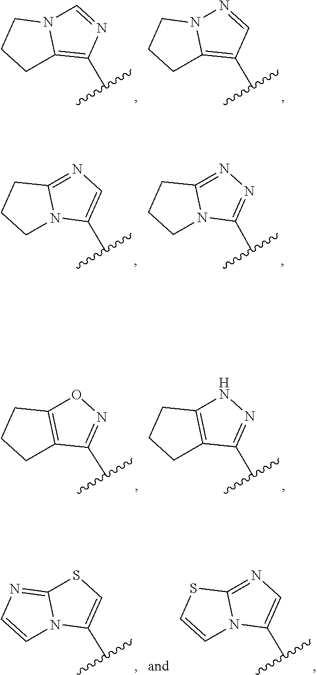
each of which is further optionally substituted with from 1-2 R^{cA}, wherein each R^{cA} is an independently selected R^{c}.

16. The compound of any one of claims 1-3 or 12, wherein R⁵ is bicyclic heteroaryl including 9 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

17. The compound of any one of claims 1-3, 12 or 16, wherein R⁵ is imidazolopyridinyl, pyrazolopyridinyl, or benzotriazolyl, each of which is optionally substituted with from 1-2 R^{cA}, wherein each R^{cA} is an independently selected R^{c}.

18. The compound of any one of claims 1-3, 12 or 16-17, wherein R⁵ is 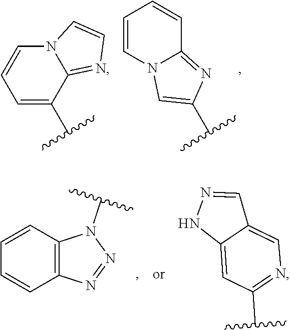
each of which is optionally substituted with from 1-2 R^{cA}, wherein each R^{cA} is an independently selected R^{c}.

19. The compound of any one of claims 1-3, wherein R⁵ is bicyclic 10-membered heteroaryl, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

20. The compound of any one of claims 3-19, wherein each R^{cA} is independently selected from the group consisting of: halo; cyano; —OH; C₁₋₆ alkyl which is optionally substituted with from 1-6 independently selected R^{a}; C₁₋₄ alkoxy optionally substituted with C₁₋₄ alkoxy or C₁₋₄ haloalkoxy; C₁₋₄ haloalkoxy; and —C(═O)NR′R″.

21. The compound of any one of claims 3-20, wherein one occurrence of R^{cA} is an independently selected halo, such as —F or —Cl.

22. The compound of any one of claims 3-21, wherein one occurrence of R^{cA} is cyano.

23. The compound of any one of claims 3-22, wherein one occurrence of R^{cA} is C₁₋₆ alkyl which is optionally substituted with from 1-6 independently selected R^{a}.

24. The compound of any one of claims 3-23, wherein one occurrence of R^{cA} is C₁₋₆ alkyl, such as C₁₋₃ alkyl.

25. The compound of any one of claims 3-23, wherein one occurrence of R^{cA} is C₁₋₆ alkyl substituted with —OH or —NR^{e}R^{f}, such as C₁₋₃ alkyl substituted with —OH or NH₂.

26. The compound of any one of claims 3-25, wherein one occurrence of R^{cA} is C₁₋₄ alkoxy optionally substituted with C₁₋₄ alkoxy or C₁₋₄ haloalkoxy, such as wherein one occurrence of R^{cA} is C₁₋₄ alkoxy, such as methoxy or ethoxy.

27. The compound of any one of claims 3-26, wherein one occurrence of R^{cA} is —C(═O)NR′R″, such as C(═O)NH₂.

28. The compound of claims 1 or 2, wherein R⁵ is 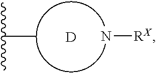
wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c}.

29. The compound of any one of claims 1-2 or 28, wherein R⁵ is 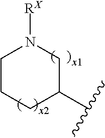
which is optionally substituted with from 1-2 R^{c}, wherein x1 and x2 are each independently 0, 1, or 2.

30. The compound of claim 29, wherein x1=0, and x2=0.

31. The compound of claim 29, wherein x1=0, and x2=1.

32. The compound of claim 29, wherein x1=0, and x2=2.

33. The compound of any one of claims 1-2 or 28-29, wherein R⁵ is selected from the group consisting of:

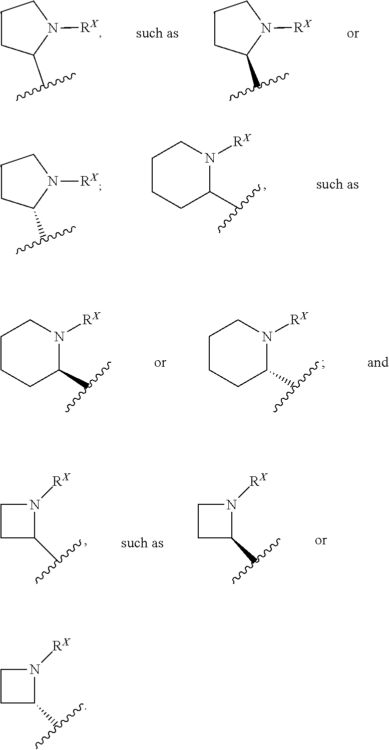

34. The compound of any one of claims 28-33, wherein R^{X} is C(═O)(C₁₋₄ alkyl) or S(O)₂(C₁₋₄ alkyl).

35. The compound of any one of claims 28-34, wherein R^{X} is C(═O)(C₁₋₄ alkyl), such as C(═O)Me or C(═O)Et.

36. The compound of any one of claims 28-34, wherein R^{X} is S(O)₂(C₁₋₄ alkyl), such as S(O)₂Me.

37. The compound of claims 1 or 2, wherein R⁵ is —R^{g2}—R^{W}.

38. The compound of any one of claims 1-2 or 37, wherein R⁵ is —R^{g2}—R^{W}; and the —R^{g2} present in —R^{g2}—R^{W} is heterocyclylene or heterocycloalkenylene including from 3-10 ring atoms, wherein from 1-3 ring atoms are heteroatoms, each independently selected from the group consisting of N, N(H), N(R^{d}), O, and S(O)₀₋₂, and wherein the heterocyclylene or heterocycloalkenylene is optionally substituted with from 1-4 substituents independently selected from the group consisting of oxo and R^{c}.

39. The compound of any one of claims 1-2 or 37-38, wherein —R⁵ is 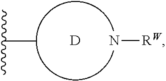
wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), 0, and S(O)₀₋₂, and wherein the heterocyclylene is optionally substituted with from 1-3 substituents each independently selected from the group consisting of: oxo and —R^{c}.

40. The compound of any one of claims 1-2 or 37-39, wherein —R⁵ is 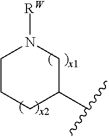
optionally substituted with from 1-2 R^{c}, wherein x1 and x2 are each independently 0, 1, or 2.

41. The compound of claim 40, wherein x1=0, and x2=0.

42. The compound of claim 40, wherein x1=0, and x2=1; or x1=0, and x2=2.

43. The compound of any one of claims 1-2 or 37-42, wherein R⁵ is selected from the group consisting of:

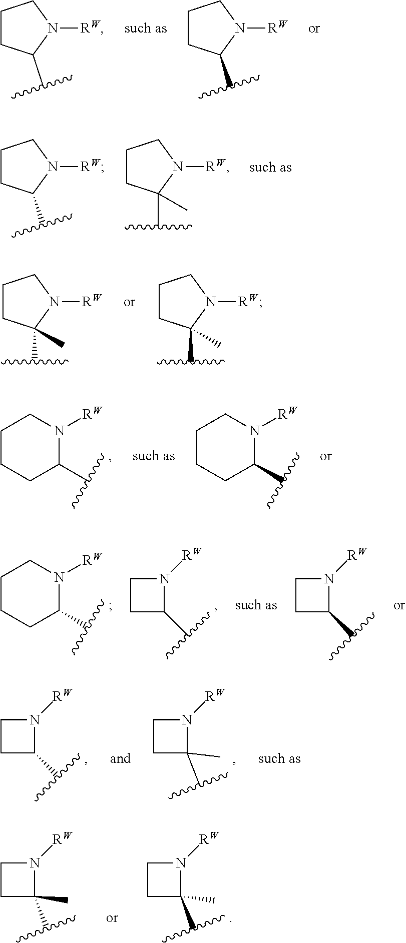

44. The compound of any one of claims 1 or 2 wherein R⁵ is R^{W}.

45. The compound of any one of claims 37-44, wherein R^{W} is -L^{W}-W; and L^{W} is C(═O) NHC(═O)*, or NHS(O)₁₋₂* wherein the asterisk represents point of attachment to W.

46. The compound of any one of claims 37-45, wherein W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom.

47. The compound of claim 37-46, wherein W is C₂₋₄ alkenyl or C₂₋₄ alkynyl optionally substituted with from 1-3 R^{a} and further optionally substituted with R^{g}, wherein W is attached to L^{W} via an sp² or sp hybridized carbon atom.

48. The compound of claim 37-47, wherein W is CH═CH₂, CH═CHCH₂NMe₂, or

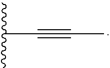 [SMILES: *C#CC]

49. The compound of any one of claims 37-48, wherein -L^{W}-W is —C(═O)CH═CH₂, —C(═O)CH═CHCH₂NMe₂, or

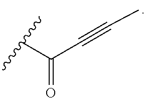 [SMILES: *C(=O)C#CC]

50. The compound of claims 1 or 2, wherein R⁵ is —R^{g2}—R^{Y}.

51. The compound of any one of claims 1-2 or 50, wherein R⁵ is —R^{g2}—R^{Y}, wherein the —R^{g2} present in —R^{g2}—R is heterocyclylene or heterocycloalkenylene including from 3-10 ring atoms, wherein from 1-3 ring atoms are heteroatoms, each independently selected from the group consisting of N, N(H), N(R^{d}), O, and S(O)₀₋₂, and wherein the heterocyclylene or heterocycloalkenylene is optionally substituted with from 1-3 substituents independently selected from the group consisting of oxo and R^{c}.

52. The compound of any one of claims 1-2 or 50-51, wherein —R⁵ is 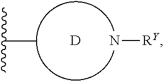
wherein Ring D is heterocyclylene including from 3-10 ring atoms, wherein from 0-2 ring atoms (in addition to the ring nitrogen atom bonded to R^{Y}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}.

53. The compound of any one of claims 1-2 or 50-52, wherein —R⁵ is 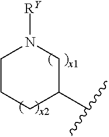
optionally substituted with from 1-2 R^{c}, wherein x1 and x2 are each independently 0, 1, or 2.

54. The compound of claim 53, wherein x1=0, and x2=0.

55. The compound of claim 53, wherein x1=0, and x2=1.

56. The compound of claim 53, wherein x1=0, and x2=2.

57. The compound of any one of claims 1-2 or 50-53, wherein R⁵ is selected from the group consisting of:

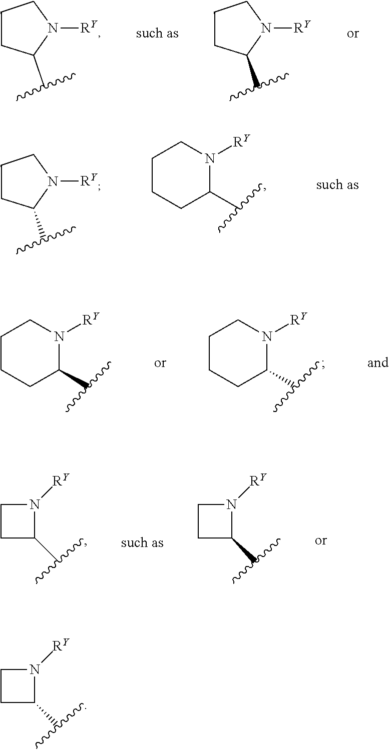

58. The compound of any one of claims 1-2 or 50, wherein R⁵ is —R^{g2}—R^{Y}; and the —R^{g2} present in —R^{g2}—R is monocyclic heteroarylene including from 5-6 ring atoms, wherein from 1-4 ring atoms are heteroatoms, each independently selected from the group consisting of N, N(H), N(R^{d}), O, and S, and wherein the heteroarylene is optionally substituted with from 1-3 R^{c}.

59. The compound of any one of claims 1-2, 50, or 58, wherein R⁵ is —R^{g2}—R^{Y}; and the —R^{g2} present in —R^{g2}—R is monocyclic heteroarylene including 5 ring atoms, wherein from 1-4 ring atoms are heteroatoms, each independently selected from the group consisting of N, N(H), N(R^{d}), O, and S, and wherein the heteroarylene is optionally substituted with from 1-2 R^{c}.

60. The compound of any one of claims 1-2, 50, or 58-59, wherein R⁵ is selected from the group consisting of:

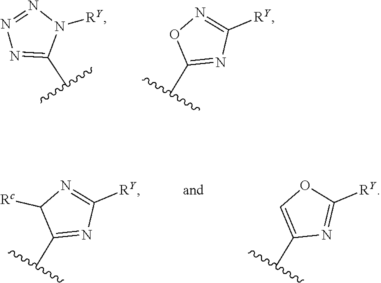

61. The compound of any one of claims 50-60, wherein —R^{Y} is —R^{g}.

62. The compound of any one of claims 50-61, wherein —R^{Y} is selected from the group consisting of:
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c}.

63. The compound of any one of claims 50-62, wherein —R^{Y} is C₆₋₁₀ aryl optionally substituted with from 1-4 R^{c}.

64. The compound of any one of claims 50-63, wherein —R^{Y} is phenyl optionally substituted with from 1-3 R^{c}.

65. The compound of any one of claims 50-62, wherein —R^{Y} is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

66. The compound of any one of claims 50-62 or 65, wherein —R^{Y} is monocyclic heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

67. The compound of any one of claims 50-62 or 65-66, wherein —R^{Y} is selected from the group consisting of pyridyl and pyrazolyl, each of which is optionally substituted with from 1-2 R^{c}.

68. The compound of claims 1 or 2, wherein R⁵ is C₃₋₁₀ cycloalkyl or C₃₋₁₀ cycloalkenyl, each optionally substituted with from 1-4 substituents each independently selected from the group consisting of: oxo and R^{c}.

69. The compound of any one of claims 1-2 or 68, wherein R⁵ is C₃₋₁₀ cycloalkyl substituted with from 1-4 substituents each independently selected from the group consisting of: oxo and R^{c}.

70. The compound of any one of claims 1-2 or 68-69, wherein R⁵ is C₃₋₆ cycloalkyl substituted with C₁₋₄ alkoxy or C₁₋₄ haloalkoxy; and R⁵ is further optionally substituted from 1-2 substituents each independently selected from the group consisting of: oxo and R^{c}.

71. The compound of any one of claims 1-2 or 68-70, wherein R⁵ is cyclopropyl that is substituted with C₁₋₄ alkoxy or C₁₋₄ haloalkoxy, such as:

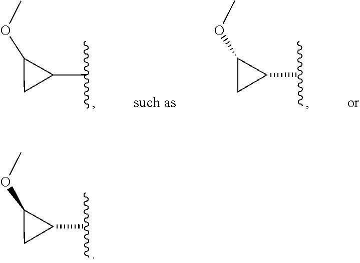

72. The compound of claims 1 or 2, wherein R⁵ is —S(O)₀₋₂(C₁₋₆ alkyl) which is optionally substituted with from 1-6 R^{a}.

73. The compound of any one of claims 1-2 or 72, wherein R⁵ is —S(O)₂(C₁₋₆ alkyl) which is optionally substituted with from 1-6 R^{a}.

74. The compound of any one of claims 1-2 or 72-73, wherein R⁵ is —S(O)₂(C₁₋₆ alkyl), such as —S(O)₂(C₁₋₃ alkyl).

75. The compound of claims 1 or 2, wherein R⁵ is selected from -L⁵-R^{g}, -L⁵-R^{g2}—R^{Y}, and -L5-R^{g2}—R^{W}.

76. The compound of any one of claims 1-2 or 75, wherein R⁵ is-L⁵-R^{g.}

77. The compound of any one of claims 1-2 or 75-76, wherein R⁵ is —O—R^{g}.

78. The compound of any one of claims 1-2 or 75-77, wherein R⁵ is —O—R^{g}; and the R^{g} present in —O—R^{g} is C₃₋₁₀ cycloalkyl or C₃₋₁₀ cycloalkenyl, each optionally substituted with from 1-4 substituents each independently selected from the group consisting of: oxo and R^{c}.

79. The compound of any one of claims 1-2 or 75-78, wherein R⁵ is —O—(C₃₋₆ cycloalkyl), wherein the C₃₋₆ cycloalkyl is optionally substituted with from 1-3 R^{c}.

80. The compound of any one of claims 1-2 or 75-79, wherein R⁵ is

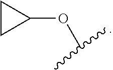 [SMILES: *OC1CC1]

81. The compound of any one of claims 1-80, wherein L¹ is C₁₋₁₀ alkylene optionally substituted with from 1-6 R^{a}.

82. The compound of any one of claims 1-81, wherein L¹ is C₁₋₆ alkylene optionally substituted with from 1-6 R^{a}.

83. The compound of any one of claims 1-82, wherein L¹ is C₁₋₄ alkylene optionally substituted with from 1-6 R^{a}.

84. The compound of any one of claims 1-83, wherein L¹ is C₁₋₄ alkylene.

85. The compound of any one of claims 1-84, wherein L¹ is —CH₂— or —CH₂CH₂—.

86. The compound of any one of claims 1-84, wherein L¹ is 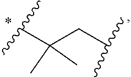 [SMILES: *CC(*)(C)C]
wherein the asterisk represents point of attachment to R^{W}.

87. The compound of any one of claims 1-80, wherein L¹ is a bond.

88. The compound of claim 1, wherein X¹ is

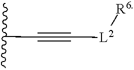

89. The compound of claims 1 or 88, wherein R⁶ is R^{g}.

90. The compound of any one of claims 1 or 88-89, wherein R⁶ is heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

91. The compound of any one of claims 1 or 88-90, wherein R⁶ is heterocyclyl including from 3-10 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}, such as: wherein R⁶ is heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-2 substituents independently selected from the group consisting of oxo and R^{c}.

92. The compound of any one of claims 1 or 88-91, wherein R⁶ is selected from the group consisting of pyrrolidinyl, piperidinyl, oxetanyl, tetrahydrofuranyl, and tetrahydropyranyl, each of which is optionally substituted with 1-2 substituents independently selected from the group consisting of oxo and R^{c}, wherein the ring nitrogen of the pyrrolidinyl or piperidinyl is optionally substituted with R^{d}, such as wherein R⁶ is

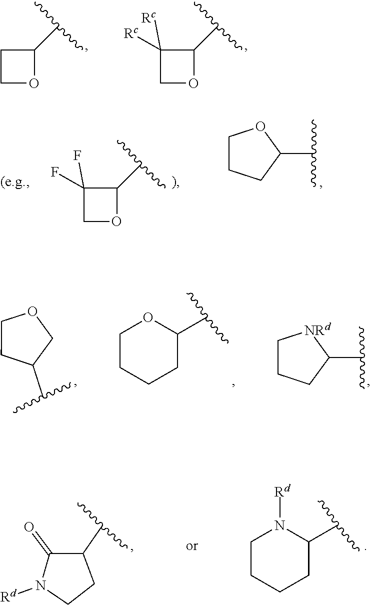

93. The compound of any one of claims 1 or 88-89, wherein R⁶ is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; such as:
wherein R⁶ is heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}, such as: wherein R⁶ is

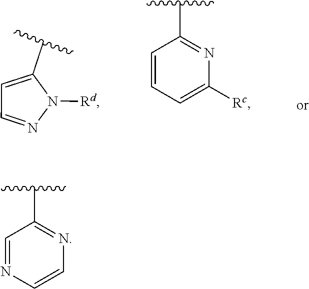

94. The compound of claims 1 or 88, wherein R⁶ is —R^{g2}—R^{W} or —R^{g2}—R^{Y}.

95. The compound of any one of claims 1, 88, or 94, wherein R⁶ is —R^{g2}—R^{W}.

96. The compound of any one of claims 1, 88, or 94-95, wherein —R⁶ is 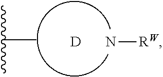
wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}; optionally wherein —R⁶ is a monocyclic heterocyclylene ring including from 3-10 ring atoms as defined above with a nitrogen atom bonded to R^{W} 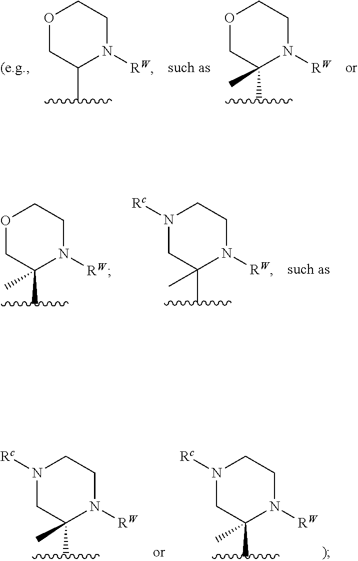
optionally wherein —R⁶ is a bicyclic heterocyclylene ring including from 3-10 ring atoms as defined above with a nitrogen atom bonded to R^{W}

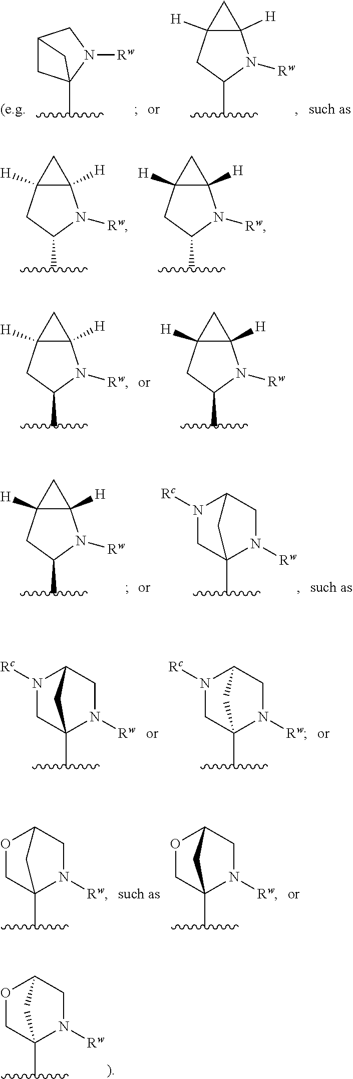

97. The compound of any one of claims 1, 88, or 94-96, wherein —R⁶ is 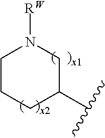
optionally substituted with from 1-2 R^{c}, wherein x1 and x2 are each independently 0, 1, or 2.

98. The compound of claim 97, wherein x1=0, and x2=0; or x1=0, and x2=1; or x1=0, and x2=2.

99. The compound of any one of claims 1, 88, or 94-98, wherein R⁶ is selected from the group consisting of:

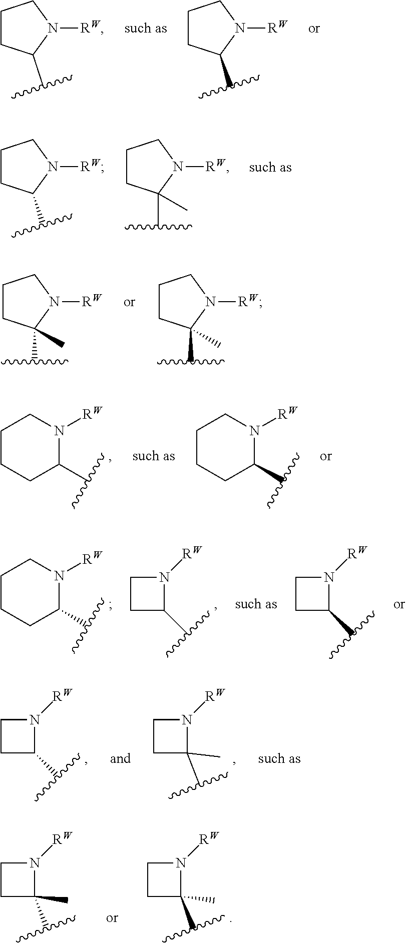

100. The compound of any one of claims 1, 88, 94-95, wherein R⁶ is C₃-C₆ cycloalkyl (e.g. cyclobutyl) substituted with R^{W}; or oxetanyl substituted with R^{W}; or tetrahydrofuryl substituted with R^{W}.

101. The compound of any one of claims 1 or 88, wherein R⁶ is —R^{W}.

102. The compound of any one of claims 94-101, wherein —R^{W} is -L^{W}-W; and L^{W} is C(═O) NHC(═O)*, NR^{d}C(═O)* (e.g., NMeC(═O)*), or NHS(O)₁₋₂* wherein the asterisk represents point of attachment to W.

103. The compound of any one of claims 94-102, wherein W is C₂₋₆ alkenyl or C₂₋₆ optionally substituted with from 1-3 R^{a} and further optionally substituted with R^{g}, wherein W is attached to L^{W} via an sp² or sp hybridized carbon atom.

104. The compound of any one of claims 94-103, wherein W is C₂₋₄ alkenyl (e.g., CH═CH₂) or C₂₋₄ alkynyl alkynyl 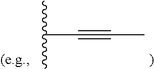 [SMILES: *C#CC]
optionally substituted with from 1-3 R^{a} and further optionally substituted with R^{g}, wherein W is attached to L^{W} via an sp² or sp hybridized carbon atom.

105. The compound of any one of claims 94-104, wherein -L^{W}-W is —C(═O)CH═CH₂; —C(═O)NHCH═CH₂; C(═O)CH═CHCH₂NR^{e}R^{f} (e.g., C(═O)CH═CHCH₂N(HMe), C(═O)CH═CHCH₂NMe₂,

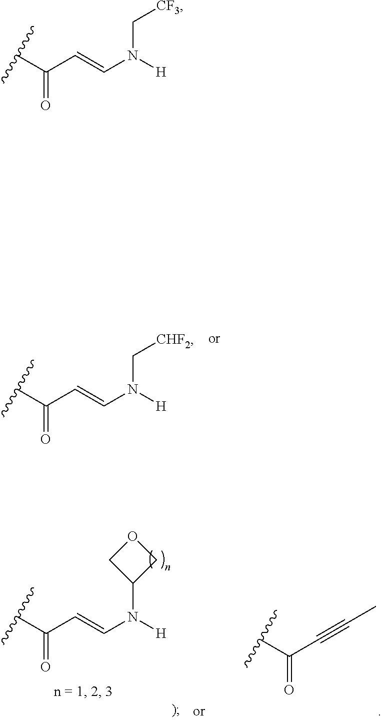

106. The compound of claims 1 or 88, wherein R⁶ is —C₁₋₆ alkoxy or —S(O)₀₋₂(C₁₋₆ alkyl), each optionally substituted with from 1-6 R^{a}.

107. The compound of any one of claims 1, 88, or 106, wherein R⁶ is —C₁₋₆ alkoxy, such as —C₁₋₃ alkoxy, such as methoxy.

108. The compound of any one of claims 1 or 88-107, wherein L² is a bond.

109. The compound of any one of claims 1 or 88-107, wherein L² is C₁₋₁₀ alkylene optionally substituted with from 1-6 R^{a}wherein R^{a} is —NR^{e}R^{f} (e.g., NMe₂), halo (e.g., fluoro), alkoxyl (e.g., methoxy).

110. The compound of any one of claims 1, 88-107, or 109, wherein L² is C₁₋₆ alkylene optionally substituted with from 1-6 R^{a}, wherein R^{a} is —NR^{e}R^{f} (e.g., NMe₂), halo (e.g., fluoro), alkoxyl (e.g., methoxy).

111. The compound of any one of claims 1, 88-107, or 109-110, wherein L² is branched C₃₋₆ alkylene optionally substituted with from 1-6 R^{a}, wherein R^{a} is —NR^{e}R^{f} (e.g., NMe₂), halo (e.g., fluoro), alkoxyl (e.g., methoxy).

112. The compound of any one of claims 1, 88-107, or 109-111, wherein L² is

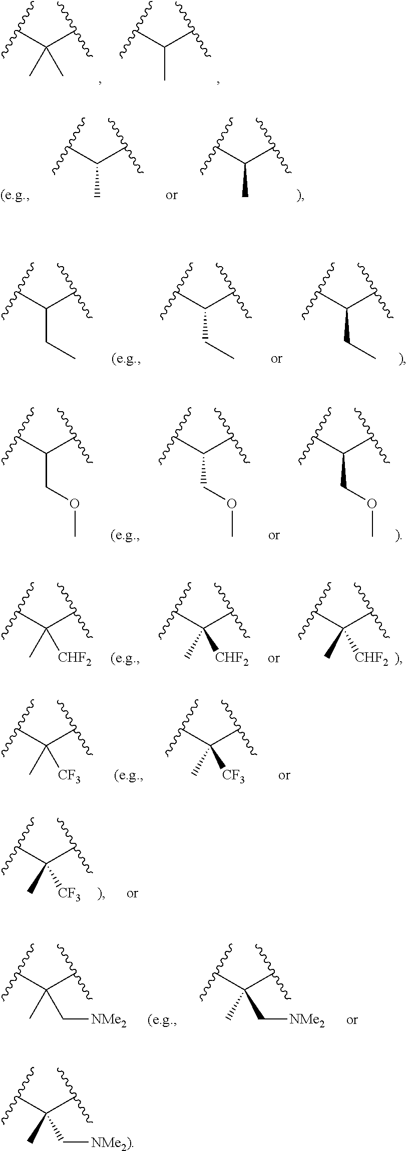

113. The compound of any one of claims 1-112, wherein n is 0.

114. The compound of any one of claims 1-112, wherein n is 1 or 2.

115. The compound of any one of claims 1-112 or 114, wherein n is 1.

116. The compound of any one of claims 1-112 or 114-115, wherein the 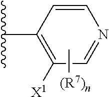
moiety is

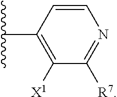

117. The compound of any one of claims 1-112 or 114-116, wherein one occurrence of R⁷ is NR^{e}R^{f}, such as: NH₂, NH(C₁₋₃ alkyl), or N(C₁₋₃ alkyl)₂.

118. The compound of any one of claims 1-112 or 114-117, wherein one occurrence of R⁷ is NH₂ or NH(C₁₋₃ alkyl), such as wherein one occurrence of R⁷ is NH₂.

119. The compound of any one of claims 1-112, wherein the 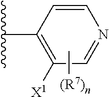
moiety is 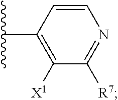
and R⁷ is NR^{e}R^{f}.

120. The compound of claim 119, wherein R⁷ is NH₂ or NH(C₁₋₃ alkyl), such as wherein R⁷ is NH₂.

121. The compound of any one of claims 1-120, wherein R^{1c} is H.

122. The compound of any one of claims 1-121, wherein R^{2a} and R^{2b} are H.

123. The compound of any one of claims 1-121, wherein from 1-2 of R^{2a} and R^{2b} is a substituent other than H.

124. The compound of claim 123, wherein one of R^{2a} and R^{2b} is C₁₋₃ alkyl optionally substituted with from 1-3 R^{a}, such as C₁₋₃ alkyl; and the other of R^{2a} and R^{2b} is H.

125. The compound of any one of claims 1-124, wherein R^{3a} and R^{3b} are H.

126. The compound of any one of claims 1-124, wherein from 1-2 of R^{3a} and R^{3b} is a substituent other than H.

127. The compound of claim 126, wherein one of R^{3a} and R^{3b} is C₁₋₃ alkyl optionally substituted with from 1-3 R^{a}, such as C₁₋₃ alkyl optionally substituted with from 1-3 —F; and the other of R^{2a} and R^{2b} is H.

128. The compound of any one of claims 1-124, wherein R^{3a} and R^{3b}, together with the Ring B ring atom to which each is attached, form a fused saturated or unsaturated ring of 3-12 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W}.

129. The compound of any one of claims 1-124 or 128, wherein R^{3a} and R^{3b}, together with the Ring B ring atom to which each is attached, form a fused saturated ring of 4-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-8 ring atoms is optionally substituted with from 1-4 substituents independently selected from the group consisting of oxo, R^{c}, and R^{W}.

130. The compound of any one of claims 1-124 or 128-129, wherein R^{3a} and R^{3b}, together with the Ring B ring atom to which each is attached, form: 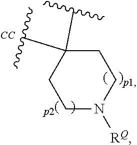
which is optionally substituted with from 1-2 substituents independently selected from the group consisting of oxo and R^{c}, wherein:
p1 and p2 are independently 0, 1, or 2;
R^{Q} is H, R^{d}, C(═O)—W, or S(O)₂W; and
cc represents the point of attachment to C(R^{2a}R^{2b}).

131. The compound of any one of claims 1-124 or 128-130, wherein R^{3a} and R^{3b}, together with the Ring B ring atom to which each is attached, form 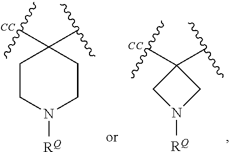
wherein R^{Q} is H, R^{d}, C(═O)—W, or S(O)₂W; and cc represents the point of attachment to C(R^{2a}R^{2b}).

132. The compound of any one of claims 1-124 or 128-130, wherein R^{3a} and R^{3b}, together with the Ring B ring atom to which each is attached, form a fused ring selected from the group consisting of: 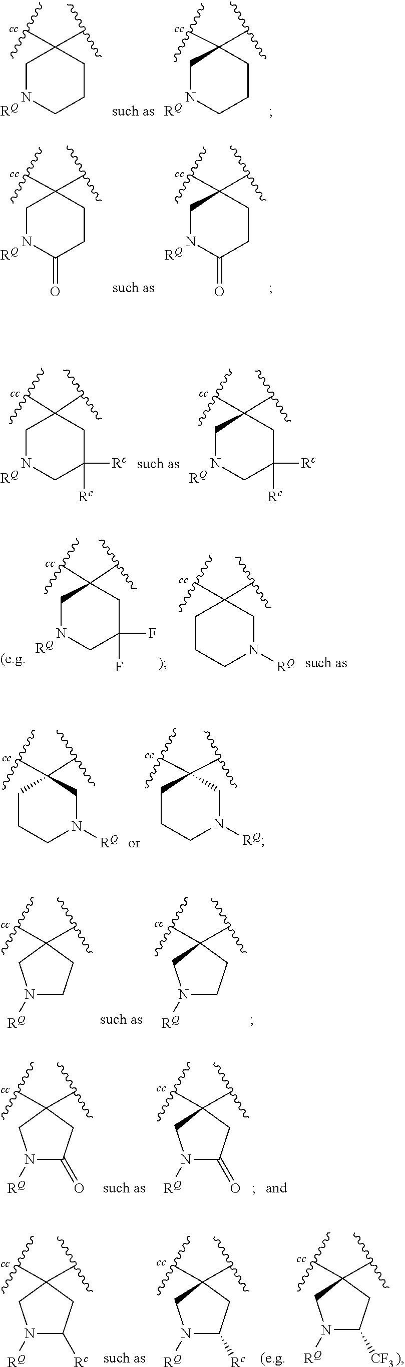
wherein R^{Q} is H, R^{d}, C(═O)—W, or S(O)₂W; and cc represents the point of attachment to C(R^{2a}R^{2b}).

133. The compound of any one of claims 130-132, wherein R^{Q} is H.

134. The compound of any one of claims 130-132, wherein R^{Q} is R^{d}.

135. The compound of any one of claims 130-132 or 134, wherein R^{Q} is C₁₋₆ alkyl optionally substituted with from 1-3 independently selected R^{a}.

136. The compound of any one of claims 130-132, wherein R^{Q} is C(═O)—W or S(O)₂W.

137. The compound of any one of claims 130-132 or 136, wherein W is C₂₋₄ alkenyl.

138. The compound of any one of claims 130-132 or 136-137, wherein R^{Q} is C(═O)—CH₂═CH₂.

139. The compound of any one of claims 1-138, wherein Ring A is 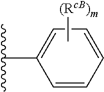
wherein each R^{cB} is an independently selected R^{c}; and m is 0, 1, 2, 3, or 4.

140. The compound of claim 139, wherein m is 1, 2, or 3.

141. The compound of claims 139 or 140, wherein m is 1 or 2, such as 2.

142. The compound of any one of claims 1-141, wherein Ring A is 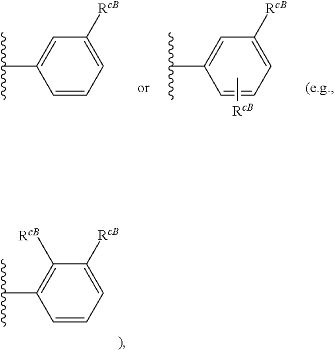
wherein each R^{cB} is an independently selected R^{c}.

143. The compound of any one of claims 139-142, wherein each R^{cB} is independently selected from the group consisting of: -halo, such as —C₁ and —F; —CN; C₁₋₄ alkoxy; C₁₋₄ haloalkoxy; C1-3 alkyl; and C₁₋₃ alkyl substituted with from 1-6 independently selected halo.

144. The compound of any one of claims 1-143, wherein Ring A is 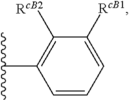
wherein R^{cB1} is R^{c}; and R^{cB2} is H or R^{c}.

145. The compound of claim 144, wherein R^{cB1} is halo, such as —F or —Cl, such as —F.

146. The compound of claims 144 or 145, wherein R^{cB2} is C₁₋₄ alkoxy or C₁₋₄ haloalkoxy, such as C₁₋₄ alkoxy, such as methoxy.

147. The compound of any one of claims 1-146, wherein Ring A is

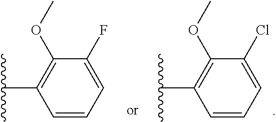

148. The compound of any one of claims 1-138, wherein Ring A is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}, such as:
wherein Ring A is bicyclic heteroaryl including from 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}, such as:
wherein Ring A is selected from the group consisting of: 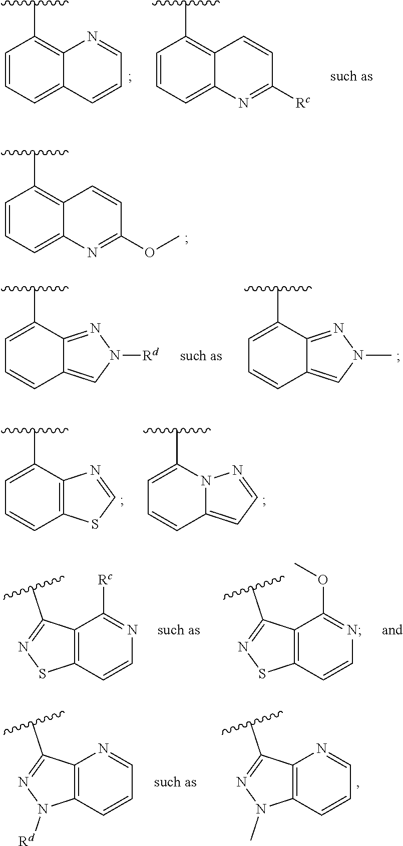
each of which is further optionally substituted with R^{c}.

149. The compound of claim 1, wherein the compound of Formula (I) is a compound of Formula (I-a) 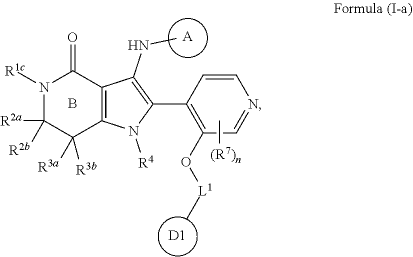
or a pharmaceutically acceptable salt thereof,
wherein Ring D1 is selected from the group consisting of:
monocyclic heteroaryl including from 5-6 ring atoms, wherein from 1-4 ring atoms are heteroatoms, each independently selected from the group consisting of N, N(H), N(R^{d}), O, and S, and wherein the heteroaryl is optionally substituted with from 1-4 R^{cA}; and
—R^{g2}—R^{Y}, wherein the —R^{g2} present in —R^{g2}—R is monocyclic heteroarylene including from 5-6 ring atoms, wherein from 1-4 ring atoms are heteroatoms, each independently selected from the group consisting of N, N(H), N(R^{d}), O, and S, and wherein the heteroarylene is optionally substituted with from 1-3 R^{cA}wherein each R^{cA} is an independently selected R^{c}; and
L¹ is a bond or C₁₋₃ alkylene optionally substituted with from 1-6 R^{a}.

150. The compound of claim 149, wherein Ring D1 is monocyclic heteroaryl including 5 ring atoms, wherein from 1-4 ring atoms are heteroatoms, each independently selected from the group consisting of N, N(H), N(R^{d}), O, and S, and wherein the heteroaryl is optionally substituted with from 1-4 R^{cA}.

151. The compound of claims 149 or 150, wherein Ring D1 is selected from the group consisting of: 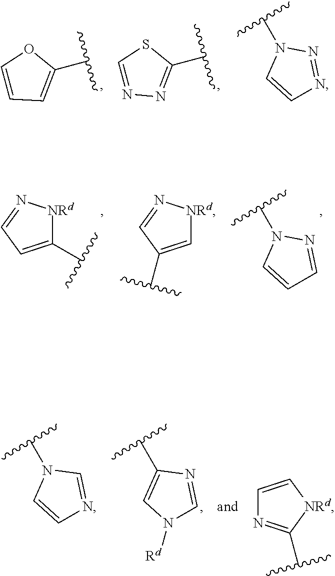
each optionally substituted with from 1-2 R^{cA}.

152. The compound of claim 149, wherein Ring D1 monocyclic heteroaryl including 6 ring atoms, wherein from 1-4 ring atoms are ring nitrogen atoms, and wherein the heteroaryl is optionally substituted with from 1-4 R^{cA}.

153. The compound of claims 149 or 152, wherein Ring D1 is selected from the group consisting of: 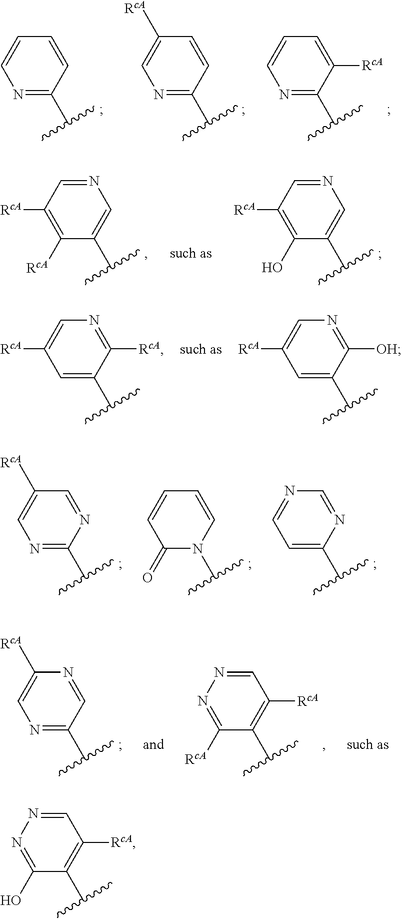
each of which is further optionally substituted with R^{cA}.

154. The compound of claim 149, wherein Ring D1 is —R^{g2}—R^{Y}; and the —R^{g2} present in —R^{g2}—R is monocyclic heteroarylene including from 5-6 ring atoms, wherein from 1-4 ring atoms are heteroatoms, each independently selected from the group consisting of N, N(H), N(R^{d}), O, and S, and wherein the heteroarylene is optionally substituted with from 1-3 R^{cA}.

155. The compound of claims 149 or 154, wherein Ring D1 is —R^{g2}—R^{Y}; and the —R^{g2} present in —R^{g2}—R is monocyclic heteroarylene including 5 ring atoms, wherein from 1-4 ring atoms are heteroatoms, each independently selected from the group consisting of N, N(H), N(R^{d}), O, and S, and wherein the heteroarylene is optionally substituted with from 1-2 R^{cA}.

156. The compound of any one claims 149-155, wherein R^{Y} is selected from the group consisting of:
phenyl optionally substituted with from 1-3 R^{c}; and
monocyclic heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

157. The compound of any one of claims 149-156, wherein n is 0.

158. The compound of any one of claims 149-156, wherein n is 1 or 2, such as n is 1.

159. The compound of any one of claims 149-156 or 158, wherein

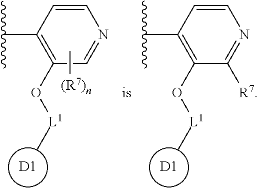

160. The compound of any one of claims 149-156 or 158-159, wherein R⁷ is NR^{e}R^{f}, such as NH₂, NH(C₁₋₃ alkyl), or N(C₁₋₃ alkyl)₂, such as wherein R⁷ is NH₂.

161. The compound of claim 1, wherein the compound of Formula (I) is a compound of Formula (I-b) 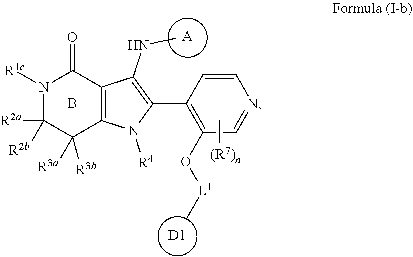
or a pharmaceutically acceptable salt thereof,
wherein Ring D2 is bicyclic heteroaryl including from 8-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}; and
L¹ is a bond or C₁₋₃ alkylene optionally substituted with from 1-6 R^{a}.

162. The compound of claim 161, wherein Ring D2 is heteroaryl including 8 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

163. The compound of claims 161 or 162, wherein Ring D2 is selected from the group consisting of: 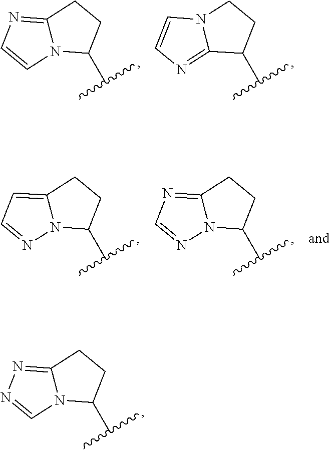 [SMILES: *C1CCc2nccn21 ; *C1CCn2ccnc21 ; *C1CCc2ccnn21 ; *C1CCc2ncnn21 ; *C1CCc2nncn21]
each of which is further optionally substituted with from 1-2 R^{cA}, wherein each R^{cA} is an independently selected R^{c}.

164. The compound of claims 161 or 162, wherein Ring D2 is selected from the group consisting of: 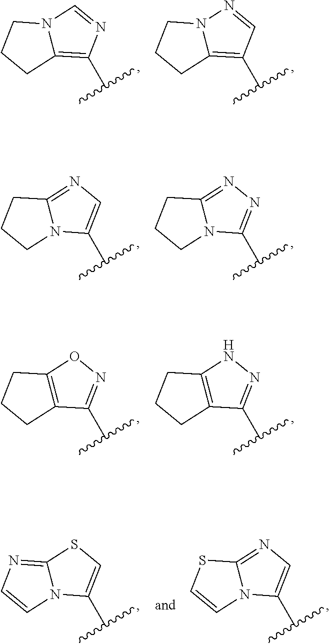
each of which is further optionally substituted with from 1-2 R^{cA}, wherein each R^{cA} is an independently selected R^{c}.

165. The compound of claim 161, wherein Ring D2 is bicyclic heteroaryl including 9 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

166. The compound of claims 161 or 165, wherein Ring D2 is imidazolopyridinyl, pyrazolopyridinyl, or benzotriazolyl, each of which is optionally substituted with from 1-2 R^{cA}, wherein each R^{cA} is an independently selected R^{c}.

167. The compound of any one of claims 161 or 165-166, wherein Ring D2 is 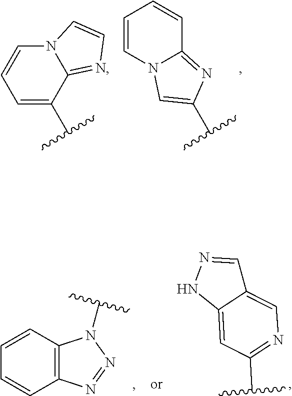
each of which is optionally substituted with from 1-2 R^{cA}, wherein each R^{cA} is an independently selected R^{c}.

168. The compound of any one of claims 161-167, wherein n is 0.

169. The compound of any one of claims 161-167, wherein n is 1 or 2, such as n is 1.

170. The compound of any one of claims 161-167 or 169, wherein

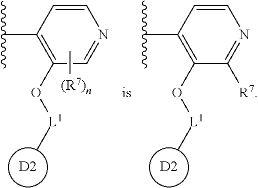

171. The compound of any one of claims 161-167 or 169-170, wherein R⁷ is NR^{e}R^{f}, such as NH₂, NH(C₁₋₃ alkyl), or N(C₁₋₃ alkyl)₂, such as wherein R⁷ is NH₂.

172. The compound of any one of claims 149-171, wherein each R^{cA} is independently selected from the group consisting of: halo; cyano; —OH; C₁₋₆ alkyl which is optionally substituted with from 1-6 independently selected R^{a}; C₁₋₄ alkoxy optionally substituted with C₁₋₄ alkoxy or C₁₋₄ haloalkoxy; C₁₋₄ haloalkoxy; and —C(═O)NR′R″.

173. The compound of claim 1, wherein the compound of Formula (I) is a compound of Formula (I-c) 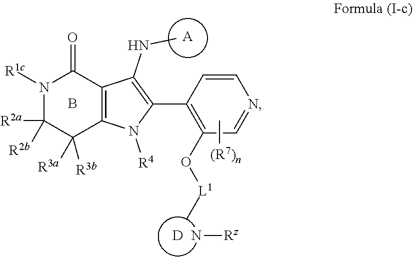
or a pharmaceutically acceptable salt thereof,
wherein Ring D is heterocyclylene including from 3-10 ring atoms, wherein from 0-2 ring atoms (in addition to the ring nitrogen atom bonded to R^{Z}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c};
R^{Z} is R^{X} or R^{Y}; and
L¹ is a bond or C₁₋₃ alkylene optionally substituted with from 1-6 R^{a}.

174. The compound of claim 173, wherein R^{Z} is R^{X}.

175. The compound of claims 173 or 174, wherein R^{Z} is C(═O)(C₁₋₄ alkyl).

176. The compound of claims 173 or 174, wherein R^{Z} is S(O)₂(C₁₋₄ alkyl).

177. The compound of claim 173, wherein R^{Z} is R^{Y}.

178. The compound of claims 173 or 177, wherein R^{Z} is R^{g}.

179. The compound of any one of claims 173 or 177-178, wherein R^{Z} is selected from the group consisting of:
phenyl optionally substituted with from 1-3 R^{c}; and
monocyclic heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

180. The compound of any one of claims 173-179, wherein n is 0.

181. The compound of any one of claims 173-179, wherein n is 1 or 2, such as wherein n is 1.

182. The compound of any one of claims 173-179 or 181, wherein

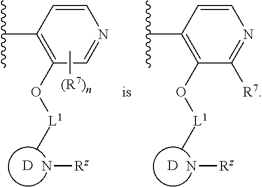

183. The compound of any one of claims 173-179 or 181-182, wherein R⁷ is NR^{e}R^{f}, such as NH₂, NH(C₁₋₃ alkyl), or N(C₁₋₃ alkyl)₂, such as wherein R⁷ is NH₂.

184. The compound of claim 1, wherein the compound of Formula (I) is a compound of Formula (I-d) 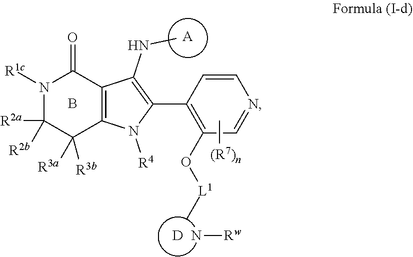
or a pharmaceutically acceptable salt thereof,
wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}; and
L¹ is a bond or C₁₋₃ alkylene optionally substituted with from 1-6 R^{a}.

185. The compound of claim 184, wherein R^{W} is -L^{W}-W; and L^{W} is C(═O).

186. The compound of claims 184 or 185, wherein W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom.

187. The compound of any one of claims 184-186, wherein W is CH═CH₂, CH═CHCH₂NMe₂, or

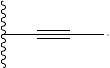 [SMILES: *C#CC]

188. The compound of any one of claims 184-187, wherein n is 0.

189. The compound of any one of claims 184-187, wherein n is 1 or 2, such as wherein n is 1.

190. The compound of any one of claims 184-187 or 189, wherein

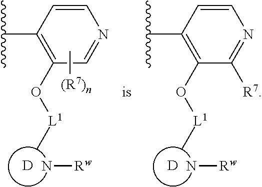

191. The compound of any one of claims 184-187 or 189-190, wherein R⁷ is NR^{e}R^{f}, such as NH₂, NH(C₁₋₃ alkyl), or N(C₁₋₃ alkyl)₂, such as wherein R⁷ is NH₂.

192. The compound of any one of claims 173-191, wherein Ring D is 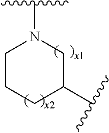
which is optionally substituted with from 1-2 R^{c}, wherein x1 and x2 are each independently 0, 1, or 2.

193. The compound of claim 192, wherein x1 is 0.

194. The compound of any one of claims 173-193, wherein Ring D is selected from the group consisting of such as or such as

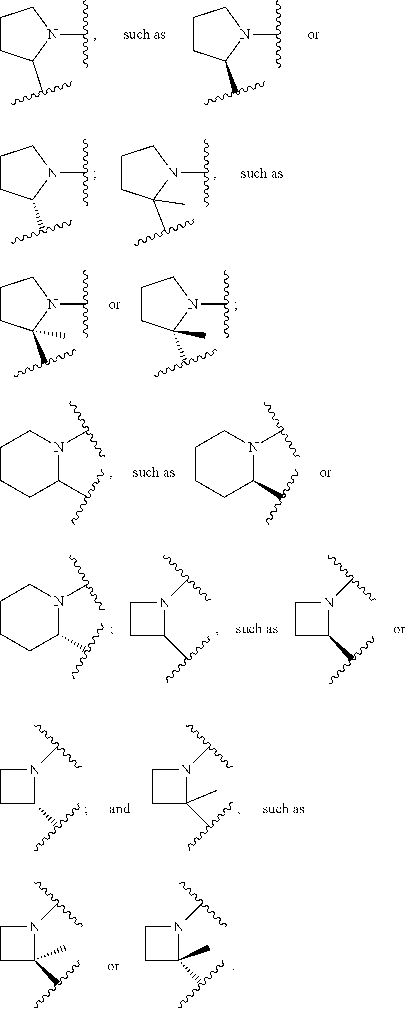

195. The compound of claim 1, wherein the compound of Formula (I) is a compound of Formula (I-e): 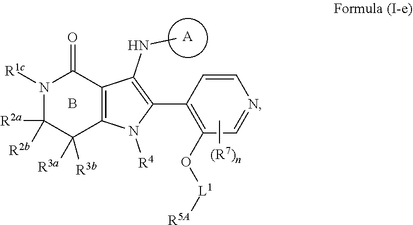
or a pharmaceutically acceptable salt thereof,
wherein R^{5A} is -L⁵-R^{g} or —S(O)₀₋₂(C₁₋₆ alkyl) which is optionally substituted with from 1-6 R^{a}; and
L¹ is C₁₋₁₀ alkylene optionally substituted with from 1-6 R^{a}.

196. The compound of claim 195, wherein R^{5A} is -L5-R^{g}.

197. The compound of claims 195 or 196, wherein R^{5A} is —O—R^{g.}

198. The compound of any one of claims 195-197, wherein R^{5A} is —O—R^{g}; and the R^{g} present in —O—R^{g} is C₃₋₁₀ cycloalkyl or C₃₋₁₀ cycloalkenyl, each optionally substituted with from 1-4 substituents each independently selected from the group consisting of: oxo and R^{c}.

199. The compound of any one of claims 195-198, wherein R^{5A} is —O—(C₃₋₆ cycloalkyl), wherein the C₃₋₆ cycloalkyl is optionally substituted with from 1-3 R^{c}, such as wherein R⁵ is

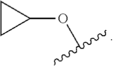 [SMILES: *OC1CC1]

200. The compound of claim 195, wherein R^{5A} is —S(O)₀₋₂(C₁₋₆ alkyl) which is optionally substituted with from 1-6 R^{a}.

201. The compound of claims 195 or 200, wherein R^{5A} is —S(O)₂(C₁₋₆ alkyl) which is optionally substituted with from 1-6 R^{a}.

202. The compound of any one of claims 195 or 200-201, wherein R^{5A} is —S(O)₂(C₁₋₃ alkyl), such as —S(O)₂Me.

203. The compound of any one of claims 195-202, wherein n is 0.

204. The compound of any one of claims 195-202, wherein n is 1 or 2, such as wherein n is 1.

205. The compound of any one of claims 195-202 or 204, wherein

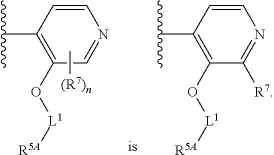

206. The compound of any one of claims 195-202 or 204-205, wherein R⁷ is NR^{e}R^{f}, such as NH₂, NH(C₁₋₃ alkyl), or N(C₁₋₃ alkyl)₂, such as wherein R⁷ is NH₂.

207. The compound of any one of claims 149-206, wherein L¹ is C₁₋₃ alkylene optionally substituted with from 1-6 R^{a}.

208. The compound of any one of claims 149-207, wherein L¹ is C₁₋₃ alkylene.

209. The compound of any one of claims 149-208, wherein L¹ is —CH₂—.

210. The compound of any one of claims 149-208, wherein L¹ is —CH₂CH₂—.

211. The compound of any one of claims 149-194, wherein L¹ is a bond.

212. The compound of claim 1, wherein the compound of Formula (I) is a compound of Formula (I-f): 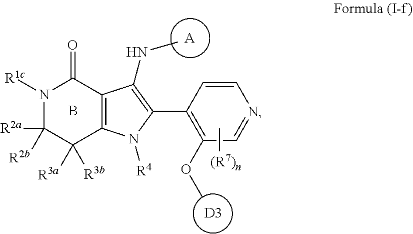
or a pharmaceutically acceptable salt thereof,
wherein Ring D3 is C₃₋₁₀ cycloalkyl substituted with from 1-4 substituents each independently selected from the group consisting of: oxo and R^{c}.

213. The compound of claim 212, wherein Ring D3 is C₃₋₆ cycloalkyl substituted with C₁₋₄ alkoxy or C₁₋₄ haloalkoxy; and R⁵ is further optionally substituted from 1-2 substituents each independently selected from the group consisting of: oxo and R^{c}.

214. The compound of claims 212 or 213, wherein R⁵ is cyclopropyl that is substituted with C₁₋₄ alkoxy or C₁₋₄ haloalkoxy, such as:

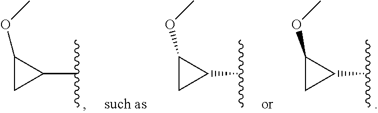

215. The compound of any one of claims 212-214, wherein n is 0.

216. The compound of any one of claims 212-214, wherein n is 1 or 2, such as wherein n is 1.

217. The compound of any one of claims 212-214 or 216, wherein

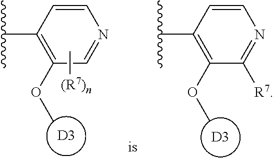

218. The compound of any one of claims 212-214 or 216-217, wherein R⁷ is NR^{e}R^{f}, such as NH₂, NH(C₁₋₃ alkyl), or N(C₁₋₃ alkyl)₂, such as wherein R⁷ is NH₂.

219. The compound of claim 1, wherein the compound of Formula (I) is a compound of Formula (I-g): 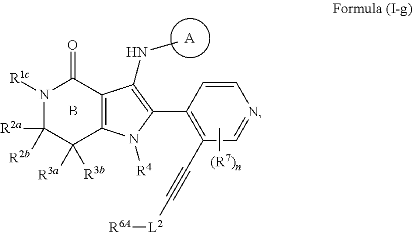
or a pharmaceutically acceptable salt thereof,
wherein L² is C₁₋₆ alkylene optionally substituted with from 1-6 R^{a}; and
R^{6A} is selected from the group consisting of —C₁₋₆ alkoxy optionally substituted with from 1-6 R^{a}; NR^{e}R^{f}; H; halo; and —OH.

220. The compound of claim 219, wherein R^{6A} is —C₁₋₆ alkoxy optionally substituted with from 1-6 R^{a}.

221. The compound of claims 219 or 220, wherein R^{6A} is —C₁₋₃ alkoxy.

222. The compound of claim 219, wherein R^{6A} is NR^{e}R^{f}.

223. The compound of claim 219, wherein R^{6A} is H, halo, or —OH.

224. The compound of any one of claims 219-223, wherein L² is branched C₃₋₆ alkylene.

225. The compound of any one of claims 219-224, wherein L² is

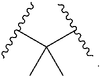 [SMILES: *C(*)(C)C]

226. The compound of any one of claims 219-223, wherein L² is C₁₋₃ alkylene, such as —CH₂—.

227. The compound of any one of claims 219-226, wherein n is 0.

228. The compound of any one of claims 219-226, wherein n is 1 or 2, such as wherein n is 1.

229. The compound of any one of claims 219-226 or 228, wherein

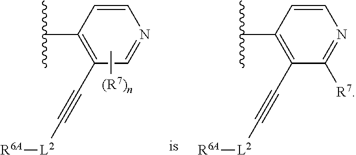

230. The compound of any one of claims 219-226 or 228-229, wherein R⁷ is NR^{e}R^{f}, such as NH₂, NH(C₁₋₃ alkyl), or N(C₁₋₃ alkyl)₂, such as wherein R⁷ is NH₂.

231. The compound of claim 1, wherein the compound of Formula (I) is a compound of Formula (I-h): 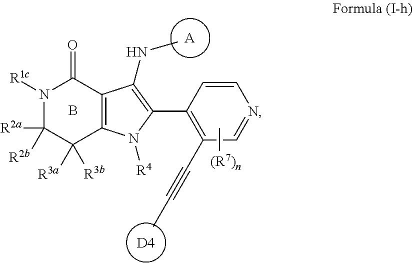
or a pharmaceutically acceptable salt thereof,
wherein Ring D4 is R^{g}.

232. The compound of claim 231, wherein Ring D4 is selected from the group consisting of:
C₃₋₁₀ cycloalkyl or C₃₋₁₀ cycloalkenyl, each of which is optionally substituted with from 1-4 substituents independently selected from the group consisting of oxo and R^{c}; and
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

233. The compound of claims 231 or 232, wherein Ring D4 is heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

234. The compound of any one of claims 231-233, wherein Ring D4 is selected from the group consisting of pyrrolidinyl, piperidinyl, oxentanyl, tetrahydrofuranyl, and tetrahydropyranyl, each of which is optionally substituted with 1-2 substituents independently selected from the group consisting of oxo and R^{c}, wherein the ring nitrogen of the pyrrolidinyl or piperidinyl is optionally substituted with R^{d}, such as wherein Ring D4 is:

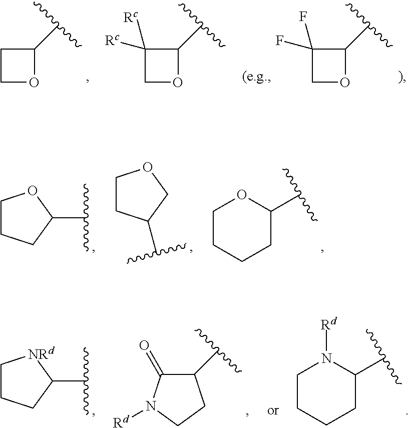

235. The compound of claim 231, wherein Ring D4 is heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}, such as: wherein R⁶ is

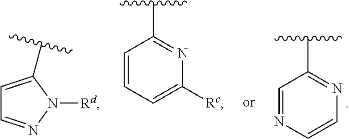

236. The compound of any one of claims 231-235, wherein n is 0.

237. The compound of any one of claims 231-235, wherein n is 1 or 2, such as wherein n is 1.

238. The compound of any one of claims 231-235 or 237, wherein

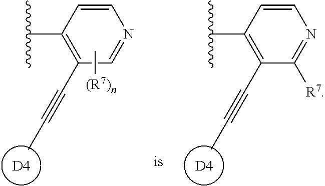

239. The compound of any one of claims 231-235 or 237-238, wherein R⁷ is NR^{e}R^{f}, such as NH₂, NH(C₁₋₃ alkyl), or N(C₁₋₃ alkyl)₂, such as wherein R⁷ is NH₂.

240. The compound of claim 1, wherein the compound of Formula (I) is a compound of Formula (I-i) 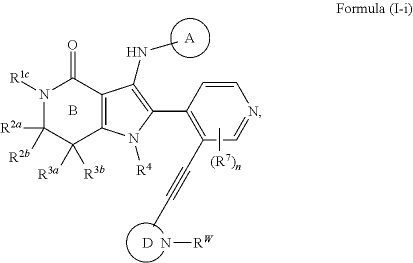
or a pharmaceutically acceptable salt thereof,
wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}.

241. The compound of claim 240, wherein R^{W} is -L^{W}-W; and L^{W} is C(═O).

242. The compound of claims 240 or 241, wherein W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom.

243. The compound of any one of claims 240-242, wherein W is CH═CH₂, CH═CHCH₂NMe₂, or

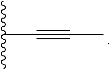 [SMILES: *C#CC]

244. The compound of any one of claims 240-243, wherein Ring D is 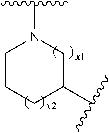
which is optionally substituted with from 1-2 R^{c}, wherein x1 and x2 are each independently 0, 1, or 2.

245. The compound of claim 244, wherein x1 is 0.

246. The compound of any one of claims 240-245, wherein Ring D is selected from the group consisting of:

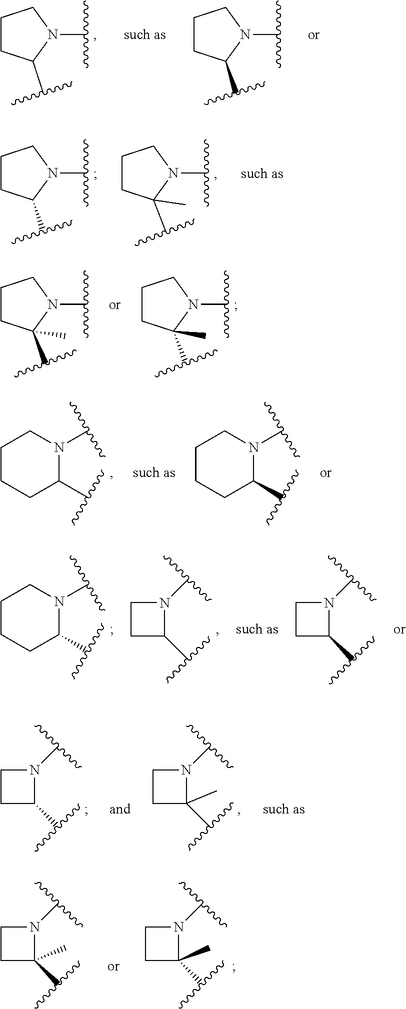

247. The compound of any one of claims 240-246, wherein n is 0.

248. The compound of any one of claims 240-246, wherein n is 1 or 2, such as wherein n is 1.

249. The compound of any one of claims 240-246 or 248, wherein

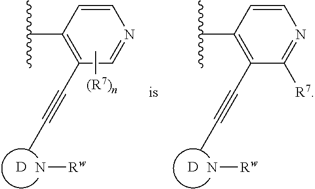

250. The compound of any one of claims 240-246 or 248, wherein R⁷ is NR^{e}R^{f}, such as NH₂, NH(C₁₋₃ alkyl), or N(C₁₋₃ alkyl)₂, such as wherein R⁷ is NH₂.

251. The compound of claim 1, wherein the compound of Formula (I) is a compound of Formula (I-j) 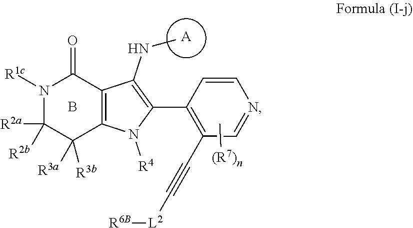
or a pharmaceutically acceptable salt thereof,
wherein L² is C₁₋₆ alkylene optionally substituted with from 1-6 R^{a}; and R^{6B} is —R^{W}.

252. The compound of claim 251, wherein R^{W} is -L^{W}-W; and L^{W} is C(═O), NHC(═O)*, or NHS(O)₁₋₂* wherein the asterisk represents point of attachment to W.

253. The compound of any one of claims 251 or 252, wherein W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom.

254. The compound of any one of claims 251-253, wherein W can be CH═CH₂, CH═CHCH₂NMe₂, or

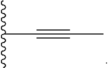 [SMILES: *C#CC]

255. The compound of any one of claims 251-254, wherein -L^{W}-W is —C(═O)CH═CH₂, —NHSO₂CH═CH₂, —C(═O)CH═CHCH₂NMe₂, or

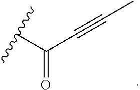 [SMILES: *C(=O)C#CC]

256. The compound of any one of claims 251-255, wherein L² is C₁₋₃ alkylene optionally substituted with from 1-6 R^{a}, wherein R^{a} is —NR^{e}R^{f} (e.g., NMe₂), halo (e.g., fluoro), or alkoxyl (e.g., methoxy).

257. The compound of any one of claims 251-256, wherein L² is

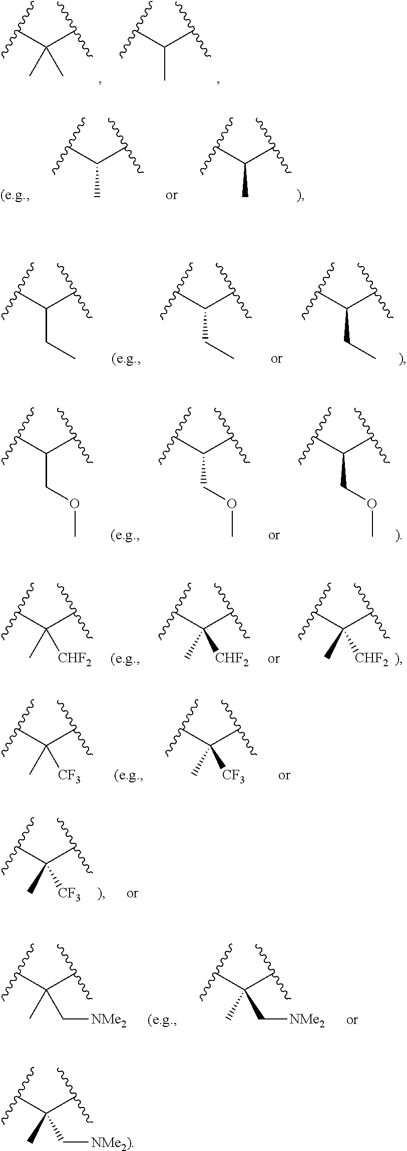

258. The compound of any one of claims 251-257, wherein n is 0.

259. The compound of any one of claims 251-257, wherein n is 1 or 2, such as wherein n is 1.

260. The compound of any one of claims 251-258, wherein

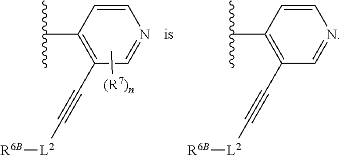

261. The compound of claim 1, wherein the compound of Formula (I) is a compound of Formula (I-k): 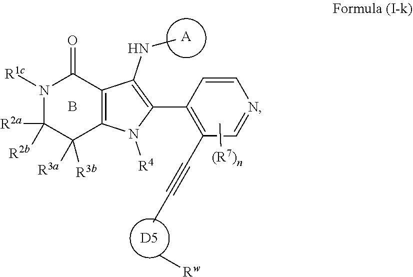
or a pharmaceutically acceptable salt thereof,
wherein Ring D5 is R^{g2}.

262. The compound of claim 261, wherein Ring D5 is selected from the group consisting of:
C₃₋₁₀ cycloalkylene or C₃₋₁₀ cycloalkenylene, each of which is optionally substituted with from 1-4 substituents independently selected from the group consisting of oxo and R^{c}; and
heterocyclylene or heterocycloalkenylene including from 3-10 ring atoms, wherein from 1-3 ring atoms are heteroatoms, each independently selected from the group consisting of N, N(H), N(R^{d}), O, and S(O)₀₋₂, and wherein the heterocyclylene or heterocycloalkenylene is optionally substituted with from 1-4 substituents independently selected from the group consisting of oxo and R^{c}.

263. The compound of claims 261 or 262, wherein Ring D5 is heterocyclylene including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

264. The compound of claims 261 or 262, wherein Ring D5 is C₃-C₆ cycloalkylene (e.g. cyclobutylene), oxetanylene, or tetrahydrofurylene.

265. The compound of any one of claims 261-264, wherein R^{W} is -L^{W}-W; and L^{W} is C(═O) or NHC(═O)*, NR^{d}C(═O)*, NHS(O)₁₋₂*, wherein the asterisk represents point of attachment to W.

266. The compound of claims 261-265, wherein W is C₂₋₆ alkenyl or C₂₋₆ alkynyl optionally substituted with from 1-3 R^{a} and further optionally substituted with R^{g}, wherein W is attached to L^{W} via an sp² or sp hybridized carbon atom.

267. The compound of any one of claims 261-266, wherein W is CH═CH₂, CH═CHCH₂NMe₂, or

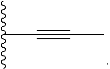 [SMILES: *C#CC]

268. The compound of any one of claims 261-267, wherein -L^{W}-W is —C(═O)CH═CH₂, —C(═O)CH═CHCH₂NMe₂, or

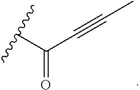 [SMILES: *C(=O)C#CC]

269. The compound of any one of claims 184-187, wherein n is 0.

270. The compound of any one of claims 184-187 or 189, wherein

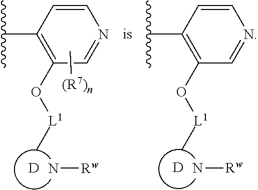

271. The compound of any one of claims 149-270, wherein R^{1c} is H.

272. The compound of any one of claims 149-271, wherein R^{2a} and R^{2b} are H.

273. The compound of any one of claims 149-272, wherein R^{3a} and R^{3b} are H.

274. The compound of any one of claims 149-272, wherein R^{3a} and R^{3b}, together with the Ring B ring atom to which each is attached, form a fused saturated ring of 4-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-8 ring atoms is optionally substituted with from 1-4 substituents independently selected from the group consisting of oxo, R^{c}, and R^{W}.

275. The compound of any one of claims 149-272 or 274, wherein R^{3a} and R^{3b}, together with the Ring B ring atom to which each is attached, form: 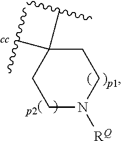
which is optionally substituted with from 1-2 substituents independently selected from the group consisting of oxo and R^{c}, wherein:
p1 and p2 are independently 0, 1, or 2;
R^{Q} is H, R^{d}, C(═O)—W, or S(O)₂W; and
cc represents the point of attachment to C(R^{2a}R^{2b}).

276. The compound of any one of claims 149-272 or 274-275, wherein R^{3a} and R^{3b}, together with the Ring B ring atom to which each is attached, form 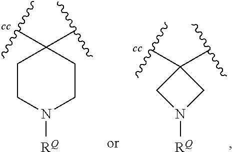
wherein R^{Q} is H, R^{d}, C(═O)—W, or S(O)₂W; and cc represents the point of attachment to C(R^{2a}R^{2b}).

277. The compound of any one of claims 149-272 or 274-275, wherein R^{3a} and R^{3b}, together with the Ring B ring atom to which each is attached, form a fused ring selected from the group consisting of: 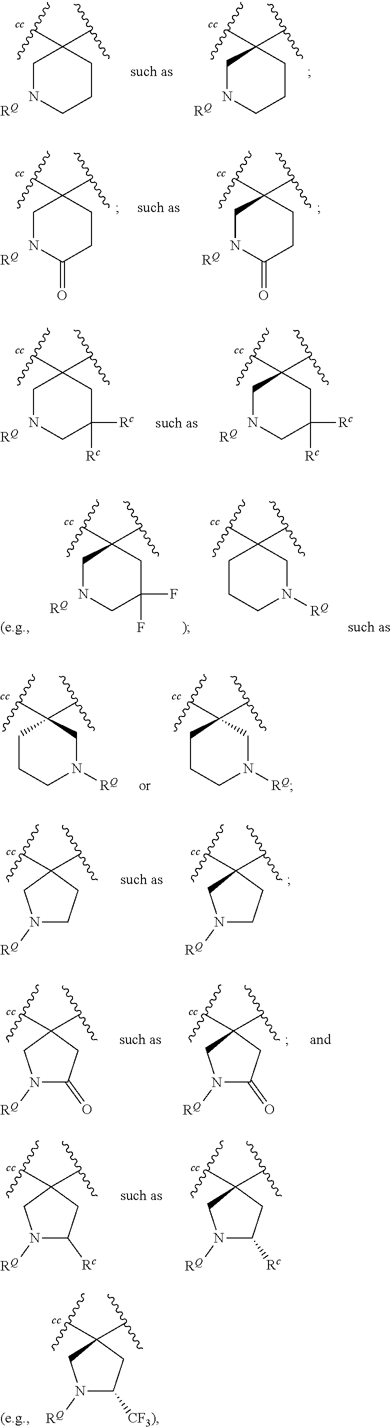
wherein R^{Q} is H, R^{d}, C(═O)—W, or S(O)₂W; and cc represents the point of attachment to C(R^{2a}R^{2b}).

278. The compound of any one of claims 275-277, wherein R^{Q} is H.

279. The compound of any one of claims 275-277, wherein R^{Q} is C₁₋₆ alkyl optionally substituted with from 1-3 independently selected R^{a}.

280. The compound of any one of claims 275-277, wherein R^{Q} is C(═O)—W or S(O)₂W, optionally wherein W is C₂₋₄ alkenyl.

281. The compound of any one of claims 275-277 or 280, wherein R^{Q} is C(═O)—CH₂═CH₂.

282. The compound of any one of claims 149-281, wherein Ring A is 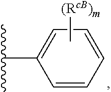
wherein each R^{cB} is an independently selected R^{c}; and m is 1, 2, or 3.

283. The compound of claim 282, wherein m is 1 or 2, such as 2.

284. The compound of any one of claims 149-283, wherein Ring A is 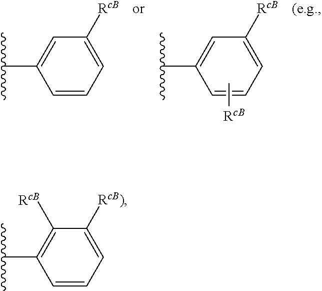
wherein each R^{cB} is independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

285. The compound of any one of claims 149-284, wherein Ring A is 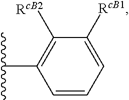
wherein R^{cB1} is R^{c}; and R^{cB2} is H or R^{c}.

286. The compound of claim 285, wherein R^{cB1} is halo, such as —F or —Cl, such as —F.

287. The compound of claims 285 or 286, wherein R^{cB2} is C₁₋₄ alkoxy or C₁₋₄ haloalkoxy, such as C₁₋₄ alkoxy, such as methoxy.

288. The compound of any one of claims 149-287, wherein Ring A is

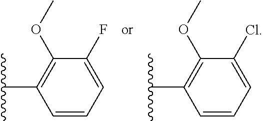

289. The compound of any one of claims 149-281, wherein Ring A is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

290. The compound of any one of claims 149-281 or 289, wherein Ring A is bicyclic heteroaryl including from 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

291. The compound of any one of claims 149-281 or 289-290, wherein Ring A is selected from the group consisting of: 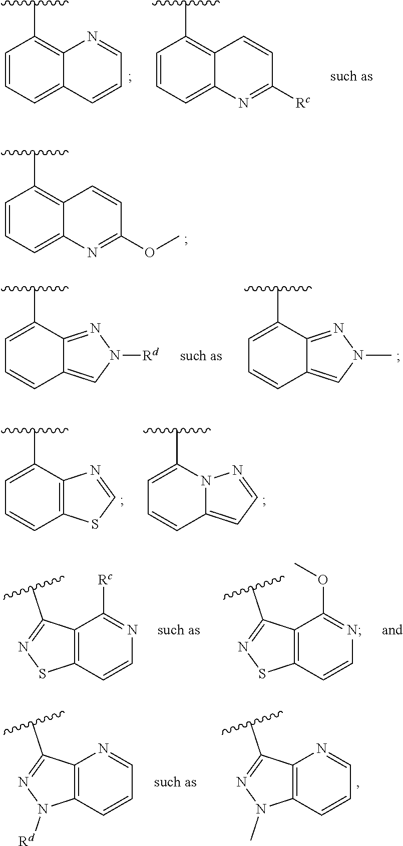
each of which is further optionally substituted with R^{c}.

292. The compound of any one of claims 1-291, wherein R⁴ is H.

293. The compound of claim 1, wherein the compound is selected from the group consisting of the compounds in Table C1, or a pharmaceutically acceptable salt thereof.

294. A pharmaceutical composition comprising a compound of any one of claims 1-293, or a pharmaceutically acceptable salt thereof, and pharmaceutically acceptable diluent or carrier.

295. A method for treating cancer in a subject in need thereof, the method comprising administering to the subject a therapeutically effective amount of a compound of any one of claims 1-293, or a pharmaceutically acceptable salt thereof, or a pharmaceutical composition according to claim 294.

296. A method for treating cancer in a subject in need thereof, the method comprising (a) determining that the cancer is associated with a dysregulation of an EGFR gene, an EGFR kinase, or expression or activity or level of any of the same; and (b) administering to the subject a therapeutically effective amount of a compound of any one of claims 1-293, or a pharmaceutically acceptable salt thereof, or a pharmaceutical composition according to claim 294.

297. A method of treating an EGFR-associated cancer in a subject, the method comprising administering to a subject identified or diagnosed as having an EGFR-associated cancer a therapeutically effective amount of a compound of any one of claims 1-293 or a pharmaceutically acceptable salt thereof, or a pharmaceutical composition according to claim 294.

298. A method of treating an EGFR-associated cancer in a subject, the method comprising:
(a) determining that the cancer in the subject is an EGFR-associated cancer; and
(b) administering to the subject a therapeutically effective amount of a compound of any one of claims 1-293 or a pharmaceutically acceptable salt thereof, or a pharmaceutical composition according to claim 294.

299. A method of treating a subject, the method comprising administering a therapeutically effective amount of a compound of any one of claims 1-293 or a pharmaceutically acceptable salt thereof, or a pharmaceutical composition according to claim 294, to a subject having a clinical record that indicates that the subject has a dysregulation of an EGFR gene, an EGFR kinase, or expression or activity or level of any of the same.

300. The method of any one of claims 296 and 298, wherein the step of determining that the cancer in the subject is an EGFR-associated cancer includes performing an assay to detect dysregulation in an EGFR gene, an EGFR kinase protein, or expression or activity or level of any of the same in a sample from the subject.

301. The method of claim 300, further comprising obtaining a sample from the subject.

302. The method of claim 301, wherein the sample is a biopsy sample.

303. The method of any one of claims 300-302, wherein the assay is selected from the group consisting of sequencing, immunohistochemistry, enzyme-linked immunosorbent assay, and fluorescence in situ hybridization (FISH).

304. The method of claim 303, wherein the FISH is break apart FISH analysis.

305. The method of claim 303, wherein the sequencing is pyrosequencing or next generation sequencing.

306. The method of any one of claims 296, 299, and 300, wherein the dysregulation in an EGFR gene, an EGFR kinase protein, or expression or activity or level of any of the same is one or more point mutations in the EGFR gene.

307. The method of claim 306, wherein the one or more point mutations in an EGFR gene results in the translation of an EGFR protein having one or more amino acid substitutions at one or more of the following amino acid positions exemplified in Table 1a and Table 1b.

308. The method of claim 307, wherein the one or more point mutations is selected from the mutations in Table 1a and Table 1b (e.g., L858R, G719S, G719C, G719A, L861Q, a deletion in exon 19 and/or an insertion in exon 20).

309. The method of claim 307, wherein the one or more point mutations is an EGFR inhibitor resistance mutation (e.g., L718Q, L747S, D761Y, T790M, C797S, T854A).

310. The method of claim 307, wherein the one or more point mutations in an EGFR gene include a deletion in exon 19 of a human EGFR gene.

311. The method of claim 307, wherein the one or more mutations is an EGFR insertion in exon 20 of a human EGFR gene.

312. The method of claim 311, wherein the insertion in exon 20 of a human EGFR gene is selected from: V769_D770insX, D770_N₇₇₁insX, N₇₇₁_P772insX, P772_H773insX, and H773_V774insX.

313. The method of claims 311 or 312, wherein the insertion in exon 20 of a human EGFR gene is selected from: Y772_A775dup, A775_G776insYVMA, G776delinsVC, G776delinsVV, V777_G778insGSP, and P780_Y781insGSP.

314. The method of any one of claims 297, 298, and 300-313, wherein the EGFR-associated cancer is selected from the group consisting of: oral cancer, oropharyngeal cancer, nasopharyngeal cancer, respiratory cancer, urogenital cancer, gastrointestinal cancer, central or peripheral nervous system tissue cancer, an endocrine or neuroendocrine cancer, a hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytomaLi-Fraumeni tumor, thyroid cancer, parathyroid cancer, pituitary tumors, adrenal gland tumors, osteogenic sarcoma tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer and skin cancer.

315. The method of any one of claims 297, 298, and 300-314, wherein the EGFR-associated cancer is selected from the group consisting of: lung cancer, pancreatic cancer, head and neck cancer, melanoma, colon cancer, renal cancer, leukemia, glioblastoma, or breast cancer.

316. The method of claim 314 or 315, wherein the lung cancer is non-small cell lung cancer.

317. The method of any one of claims 295-316, wherein the cancer is a HER2-associated cancer.

318. The method of claim 317, wherein the HER2-associated cancer is associated with a dysregulation of a HER2 gene, a HER2 kinase, or expression or activity or level of any of the same.

319. The method of any one of claims 317 and 318, wherein determining that the cancer in the subject is a HER2-associated cancer includes performing an assay to detect dysregulation in a HER2 gene, a HER2 kinase protein, or expression or activity or level of any of the same in a sample from the subject.

320. The method of claim 319, further comprising obtaining a sample from the subject.

321. The method of claim 320, wherein the sample is a biopsy sample.

322. The method of any one of claims 319-321, wherein the assay is selected from the group consisting of sequencing, immunohistochemistry, enzyme-linked immunosorbent assay, and fluorescence in situ hybridization (FISH).

323. The method of claim 322, wherein the sequencing is pyrosequencing or next generation sequencing.

324. The method of any one of claims 318-323, wherein the dysregulation in a HER2 gene, a HER2 kinase protein, or expression or activity or level of any of the same is one or more point mutations in the HER2 gene.

325. The method of claim 324, wherein the one or more point mutations in a HER2 gene results in the translation of a HER2 protein having one or more amino acid substitutions at one or more of the following amino acid positions exemplified in Table 3.

326. The method of claim 325, wherein the one or more point mutations is selected from the mutations in Table 3 (e.g., S310F, S310Y, R678Q, R678W, R678P, I767M, V773M, V777L, and V842I).

327. The method of any one of claims 295-326, wherein the cancer is selected from the group consisting of: non-small cell lung cancer, pancreatic cancer, and colorectal cancer.

328. The method of any one of claims 295-327, further comprising administering an additional therapy or therapeutic agent to the subject.

329. The method of claim 328, wherein the additional therapy or therapeutic agent is selected from radiotherapy, cytotoxic chemotherapeutics, kinase targeted-therapeutics, apoptosis modulators, signal transduction inhibitors, immune-targeted therapies, and angiogenesis-targeted therapies.

330. The method of claim 329, wherein said additional therapeutic agent is selected from one or more kinase targeted therapeutics.

331. The method of claim 330, wherein said additional therapeutic agent is a tyrosine kinase inhibitor.

332. The method of claim 331, wherein said additional therapeutic agent is a second EGFR inhibitor.

333. The method of claim 328, wherein said additional therapeutic agent is selected from osimertinib, gefitinib, erlotinib, afatinib, lapatinib, neratinib, AZD-9291, CL-387785, CO-1686, WZ4002, and combinations thereof.

334. The method of claim 328, wherein said additional therapeutic agent is a second compound of any one of claims 1-293 or a pharmaceutically acceptable salt thereof, or a pharmaceutical composition according to claim 294.

335. The method of claims 328 or 329, wherein said additional therapeutic agent is a HER2 inhibitor.

336. The method of claim 335, wherein the HER2 inhibitor is selected from trastuzumab, pertuzumab, trastuzumab emtansine, lapatinib, KU004, neratinib, dacomitinib, afatinib, tucatinib, erlotinib, pyrotinib, poziotinib, CP-724714, CUDC-101, sapitinib (AZD8931), tanespimycin (17-AAG), IPI-504, PF299, pelitinib, S-22261 1, and AEE-788.

337. The method of any one of claims 328-336, wherein the compound of any one of claims 1-293 or a pharmaceutically acceptable salt thereof, or a pharmaceutical composition according to claim 274, and the additional therapeutic agent are administered simultaneously as separate dosages.

338. The method of any one of claims 328-336, wherein the compound of any one of claims 1-293 or a pharmaceutically acceptable salt thereof, or a pharmaceutical composition according to claim 274, and the additional therapeutic agent are administered as separate dosages sequentially in any order.

339. A method of treating a subject having a cancer, wherein the method comprises:
(a) administering one or more doses of a first EGFR inhibitor to the subject for a period of time;
(b) after (a), determining whether a cancer cell in a sample obtained from the subject has at least one EGFR inhibitor resistance mutation that confers increased resistance to a cancer cell or tumor to treatment with the first EGFR inhibitor of step (a); and
(c) administering a compound of any one of claims 1-293 or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first EGFR inhibitor of step (a); or
(d) administering additional doses of the first EGFR inhibitor of step (a) to the subject if the subject has not been determined to have a cancer cell that has at least one EGFR inhibitor resistance mutation that confers increased resistance to a cancer cell or tumor to treatment with the first EGFR inhibitor of step (a).

340. The method of claim 339, wherein the anticancer agent in step (c) is a second EGFR inhibitor, an immunotherapy, a HER2 inhibitor, or a combination thereof.

341. The method of claim 339, wherein the anticancer agent in step (c) is the first EGFR inhibitor administered in step (a).

342. The method of claim 339, wherein the subject is administered additional doses of the first inhibitor of EGFR of step (a), and the method further comprises (e) administering another anticancer agent to the subject.

343. The method of claim 342, wherein the anticancer agent of step (e) is a second EGFR inhibitor, an immunotherapy, or a combination thereof.

344. The method of claim 342, wherein the anticancer agent of step (e) is a compound of any one of claims 1-313 or a pharmaceutically acceptable salt thereof.

345. The method of any one of claims 339-344, wherein the EGFR inhibitor resistance mutation is a substitution at amino acid position 718, 747, 761, 790, 797, or 854 (e.g., L718Q, L747S, D761Y, T790M, C797S, T854A).

346. A method of treating an EGFR-associated cancer in a subject, the method comprising administering to a subject identified or diagnosed as having an EGFR-associated cancer that has one or more EGFR inhibitor resistance mutations a therapeutically effective amount of a compound of any one of claims 1-313 or a pharmaceutically acceptable salt thereof, or a pharmaceutical composition according to claim 294.

347. A method of treating an EGFR-associated cancer in a subject, the method comprising:
(a) determining that the cancer in the subject has one or more EGFR inhibitor resistance mutations; and
(b) administering to the subject a therapeutically effective amount of a compound of any one of claims 1-293 or a pharmaceutically acceptable salt thereof, or a pharmaceutical composition according to claim 294.

348. A method of treating a subject having a cancer, wherein the method comprises:
(a) determining whether a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a compound of any one of claims 1-293 or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first modulator of EGFR that was previously administered to the subject; or
(c) administering additional doses of the first modulator of EGFR to the subject if the subject has not been determined to have a cancer cell that has at least one EGFR modulator resistance mutation that confers increased resistance to a cancer cell or tumor to treatment with the first modulator of EGFR previously administered to the subject.

349. The method of claim 348, wherein the anticancer agent of step (b) is a second EGFR inhibitor, an immunotherapy, a HER2 inhibitor, or a combination thereof.

350. The method of claim 348, wherein the anticancer agent of step (b) is the first EGFR inhibitor previously administered to the subject.

351. The method of claim 348, wherein the subject is administered additional doses of the first EGFR inhibitor previously administered to the subject, and the method further comprises (d) administering another anticancer agent to the subject.

352. The method of claim 351, wherein the anticancer agent of step (d) is a second EGFR inhibitor, an immunotherapy, or a combination thereof.

353. The method of claim 351, wherein the anticancer agent of step (d) is a compound of any one of claims 1-293 or a pharmaceutically acceptable salt thereof.

354. The method of claim 353, wherein the second EGFR inhibitor is selected from osimertinib, gefitinib, erlotinib, afatinib, lapatinib, neratinib, AZD-9291, CL-387785, CO-1686, WZ4002, and combinations thereof.

355. The method of any one of claims 346-354, wherein the cancer is selected from the group consisting of: non-small cell lung cancer, pancreatic cancer, and colorectal cancer.

356. The method of any one of claims 346-355, wherein the cancer is associated with a dysregulation of a HER2 gene, a HER2 kinase, or expression or activity or level of any of the same.

357. The method of claim 356, wherein the dysregulation in a HER2 gene, a HER2 kinase protein, or expression or activity or level of any of the same is one or more point mutations in the HER2 gene.

358. The method of claim 357, wherein the one or more point mutations in a HER2 gene results in the translation of a HER2 protein having one or more amino acid substitutions at one or more of the following amino acid positions exemplified in Table 3.

359. The method of claim 358, wherein the one or more point mutations is selected from the mutations in Table 3 (e.g., S310F, S310Y, R678Q, R678W, R678P, I767M, V773M, V777L, and V842I).

360. A method for modulating EGFR in a mammalian cell, the method comprising contacting the mammalian cell with an effective amount of a compound of any one of claims 1-293, or a pharmaceutically acceptable salt thereof.

361. The method of claim 360, wherein the contacting occurs in vivo.

362. The method of claim 360, wherein the contacting occurs in vitro.

363. The method of any one of claims 360-362, wherein the mammalian cell is a mammalian cancer cell.

364. The method of claim 363, wherein the mammalian cancer cell is a mammalian EGFR-associated cancer cell.

365. The method of any one of claims 360-363, wherein the cell has a dysregulation of an EGFR gene, an EGFR kinase protein, or expression or activity or level of any of the same.

366. The method of claim 365, wherein the dysregulation in an EGFR gene, an EGFR kinase protein, or expression or activity or level of any of the same is one or more point mutations in the EGFR gene.

367. The method of claim 366, wherein the one or more point mutations in an EGFR gene results in the translation of an EGFR protein having one or more amino acid substitutions at one or more of the following amino acid positions exemplified in Table 1a and Table 1b.

368. The method of claim 366, wherein the one or more point mutations is selected from the mutations in Table 1a and Table 1b (e.g., L858R, G719S, G719C, G719A, L861Q, a deletion in exon 19 and/or an insertion in exon 20).

369. The method of claim 366, wherein the one or more point mutations is an EGFR inhibitor resistance mutation (e.g., L718Q, L747S, D761Y, T790M, C797S, T854A).

370. The method of claim 366, wherein the one or more point mutations in an EGFR gene include a deletion in exon 19 of a human EGFR gene.

371. The method of claim 366, wherein the one or more point mutations is an EGFR insertion in exon 20 of a human EGFR gene.

372. The method of claim 371, wherein the insertion in exon 20 of a human EGFR gene is selected from: A767_V769insX, V769_D770insX, D770_N₇₇₁insX, N₇₇₁_P772insX, P772_H773insX, and H773_V774insX.

373. The method of claim 372, wherein the insertion in exon 20 of a human EGFR gene is selected from: A767_V769dupASV, V769_D770insASV, D770_N₇₇₁insNPG, D770_N₇₇₁insNPY, D770_N₇₇₁insSVD, D770_N₇₇₁insGL, N₇₇₁_H773dupNPH, N₇₇₁_P772insN, N₇₇₁_P772insH, N₇₇₁_P772insV, P772_H773insDNP, P772_H773insPNP, H773_V774insNPH, H773_V774insH, H773_V774insPH, H773_V774insAH, and P772_H773insPNP.

374. A method for treating cancer in a subject in need thereof, the method comprising (a) determining that the cancer is associated with a dysregulation of a HER2 gene, a HER2 kinase, or expression or activity or level of any of the same; and (b) administering to the subject a therapeutically effective amount of a compound of any one of claims 1-293, or a pharmaceutically acceptable salt thereof, or a pharmaceutical composition according to claim 294.

375. A method of treating a HER2-associated cancer in a subject, the method comprising administering to a subject identified or diagnosed as having a HER2-associated cancer a therapeutically effective amount of a compound of any one of claims 1-293 or a pharmaceutically acceptable salt thereof, or a pharmaceutical composition according to claim 294.

376. A method of treating a HER2-associated cancer in a subject, the method comprising:
(a) determining that the cancer in the subject is a HER2-associated cancer; and
(b) administering to the subject a therapeutically effective amount of a compound of any one of claims 1-293 or a pharmaceutically acceptable salt thereof, or a pharmaceutical composition according to claim 294.

377. A method of treating a subject, the method comprising administering a therapeutically effective amount of a compound of any one of claims 1-293 or a pharmaceutically acceptable salt thereof, or a pharmaceutical composition according to claim 294, to a subject having a clinical record that indicates that the subject has a dysregulation of a HER2 gene, a HER2 kinase, or expression or activity or level of any of the same.

378. The method of any one of claims 374 and 376, wherein the step of determining that the cancer in the subject is a HER2-associated cancer includes performing an assay to detect dysregulation in a HER2 gene, a HER2 kinase protein, or expression or activity or level of any of the same in a sample from the subject.

379. The method of claim 378, further comprising obtaining a sample from the subject.

380. The method of claim 379, wherein the sample is a biopsy sample.

381. The method of any one of claims 374-380, wherein the assay is selected from the group consisting of sequencing, immunohistochemistry, enzyme-linked immunosorbent assay, and fluorescence in situ hybridization (FISH).

382. The method of claim 381, wherein the FISH is break apart FISH analysis.

383. The method of claim 381, wherein the sequencing is pyrosequencing or next generation sequencing.

384. The method of any one of claims 374, 377, and 378, wherein the dysregulation in a HER2 gene, a HER2 kinase protein, or expression or activity or level of any of the same is one or more point mutations in the HER2 gene.

385. The method of claim 384, wherein the one or more point mutations in a HER2 gene results in the translation of a HER2 protein having one or more amino acid substitutions at one or more of the following amino acid positions exemplified in Table 3.

386. The method of claim 384, wherein the one or more point mutations is selected from the mutations in Table 3 (e.g., S310F, S310Y, R678Q, R678W, R678P, I767M, V773M, V777L, and V842I).

387. The method of any one of claims 373, 376, and 377, wherein the dysregulation in a HER2 gene, a HER2 kinase protein, or expression or activity or level of any of the same is an insertion in exon 20 of the human HER2 gene.

388. The method of claim 387, wherein the insertion in exon 20 of the human HER2 gene is deletions at an amino acid position selected from: 774, 775, 776, 777, 778, and 780.

389. The method of claim 388, wherein the insertion in exon 20 of a human HER2 gene is selected from: M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777 G778insCG, G778 S779insCPG, and P780_Y781insGSP.

390. The method of any one of claims 375, 376, and 378, wherein the HER2-associated cancer is selected from the group consisting of: colon cancer, lung cancer, or breast cancer.

391. The method of claim 390, wherein the lung cancer is non-small cell lung cancer.

392. The method of any one of claims 377-391, further comprising administering an additional therapy or therapeutic agent to the subject.

393. The method of claim 392, wherein the additional therapy or therapeutic agent is selected from radiotherapy, cytotoxic chemotherapeutics, kinase targeted-therapeutics, apoptosis modulators, signal transduction inhibitors, immune-targeted therapies and angiogenesis-targeted therapies.

394. The method of claim 392, wherein said additional therapeutic agent is a second compound of any one of claims 1-293 or a pharmaceutically acceptable salt thereof, or a pharmaceutical composition according to claim 274.

395. The method of claim 392, wherein said additional therapeutic agent is selected from one or more kinase targeted therapeutics.

396. The method of claim 392, wherein said additional therapeutic agent is a tyrosine kinase inhibitor.

397. The method of claim 392, wherein said additional therapeutic agent is an EGFR inhibitor.

398. The method of claim 392, wherein said additional therapeutic agent is selected from osimertinib, gefitinib, erlotinib, afatinib, lapatinib, neratinib, AZD-9291, CL-387785, CO-1686, WZ4002, and combinations thereof.

399. The method of claim 392, wherein said additional therapeutic agent is a HER2 inhibitor.

400. The method of claim 399, wherein the HER2 inhibitor is selected from trastuzumab, pertuzumab, trastuzumab emtansine, lapatinib, KU004, neratinib, dacomitinib, afatinib, tucatinib, erlotinib, pyrotinib, poziotinib, CP-724714, CUDC-101, sapitinib (AZD8931), tanespimycin (17-AAG), IPI-504, PF299, pelitinib, S-22261 1, and AEE-788.

401. The method of any one of claims 395-400, wherein the compound of any one of claims 1-293 or a pharmaceutically acceptable salt thereof, or a pharmaceutical composition according to claim 274, and the additional therapeutic agent are administered simultaneously as separate dosages.

402. The method of any one of claims 395-400, wherein the compound of any one of claims 1-293 or a pharmaceutically acceptable salt thereof, or a pharmaceutical composition according to claim 274, and the additional therapeutic agent are administered as separate dosages sequentially in any order.
